(19) United States
(12) Patent Application Publication
(10) Pub. No.: US 2025/0234771 A1
(43) Pub. Date: Jul. 17, 2025

(54) COMPOUND, MATERIAL FOR ORGANIC ELECTROLUMINESCENCE ELEMENT, ORGANIC ELECTROLUMINESCENCE ELEMENT, AND ELECTRONIC DEVICE

(71) Applicant: IDEMITSU KOSAN CO.,LTD., Tokyo (JP)

(72) Inventors: Shota TANAKA, Chiyoda-ku (JP); Yusuke TAKAHASHI, Chiyoda-ku (JP); Hiroaki ITOI, Chiyoda-ku (JP); Takuto FUKAMI, Chiyoda-ku (JP); Tsukasa SAWATO, Chiyoda-ku (JP)

(73) Assignee: IDEMITSU KOSAN CO.,LTD., Tokyo (JP)

(21) Appl. No.: 18/729,629

(22) PCT Filed: Jan. 18, 2023
(86) PCT No.: PCT/JP2023/001326

§ 371 Date: Jul. 17, 2024

(30) Foreign Application Priority Data
Jan. 21, 2022 (JP) 2022-008248

(51) Int. Cl. H10K 85/60; H10K 50/12; H10K 50/15

(52) U.S. Cl. CPC H10K 85/633; H10K 85/636; H10K 50/12; H10K 50/156; H10K 85/615; H10K 85/622; H10K 85/626; H10K 85/6574

(57) ABSTRACT

A compound which improves properties of the organic EL device, an organic electroluminescent device in which the element properties are more improved, and an electronic device containing the organic electroluminescent device. A compound which is represented by any of formulas (1) to (3), an organic electroluminescent device including the compound, and an electronic device including the organic electroluminescent device.

### TECHNICAL FIELD

The present invention relates to a compound, a material for organic electroluminescent device, an organic electroluminescent device, and an electronic device containing the organic electroluminescent device.

### BACKGROUND ART

In general, an organic electroluminescent device (hereinafter referred to as “organic EL device”) is constituted by an anode, a cathode, and an organic layer sandwiched between the anode and cathode. When a voltage is applied between the two electrodes, electrons are injected from the cathode side and holes are injected from the anode side into the light emitting region, and the injected electrons and holes recombine in the light emitting region to generate an excited state, and when the state returns to the ground state, the light is emitted. Therefore, the development of materials which efficiently transport electrons or holes to the light emitting region and facilitate the recombination of electrons and holes is important in obtaining a high-performance organic EL device.

Patent Literatures 1 to 5 disclose compounds used as a material for organic electroluminescent device.

### CITATION LIST

### Patent Literature

PTL1: US2019/0039996A1
PTL2: KR10-2019-0005665A
PTL3: KR10-2019-0030004A
PTL4: JP6715781B2

### SUMMARY OF INVENTION

### Technical Problem

Although many compounds for organic EL devices have been reported until now, there is still a need for compounds which further improve the performance of organic EL devices.

The present invention was made to solve the above problems, and the object is to provide a compound which further improves the performance of an organic EL device, an organic EL device with further improved element performance, and an electronic device including such an organic EL device.

### Solution to Problem

As a result of extensive research about the performance of organic EL devices containing the compounds described in Patent Literatures 1 to 4, the inventors found that an organic EL device containing a compound represented by any of the following formulas (1) to (3) has more improved performance.

In one embodiment, the present invention provides a compound represented by the following formula (1),

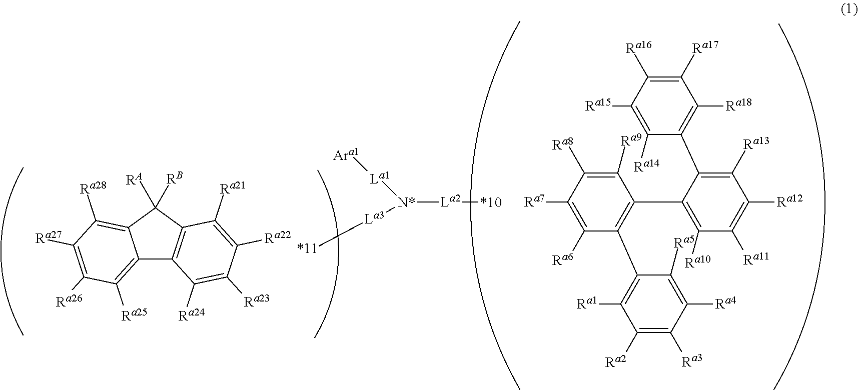

(In the formula, N* is a central nitrogen atom,
L^{a1} is a substituted or unsubstituted phenylene group, a substituted or unsubstituted naphthylene group, or a substituted or unsubstituted biphenylene group, and the substituent is an unsubstituted alkyl group having 1 to 6 carbon atoms or an unsubstituted aryl group having 6 to 12 ring carbon atoms, and no ring is condensed with the phenylene group, the biphenylene group, and the naphthylene group,
L^{a2} and L^{a3} are each independently a single bond, a substituted or unsubstituted phenylene group, a substituted or unsubstituted naphthylene group, or a substituted or unsubstituted biphenylene group, and the substituent is an unsubstituted alkyl group having 1 to 6 carbon atoms or an unsubstituted aryl group having 6 to 12 ring carbon atoms, and no ring is condensed with the phenylene group, the biphenylene group, and the naphthylene group,
one selected from R^{a1} to R^{a5} is a single bond bonded to *10, and R^{a1} to R^{a5} which are not a single bond bonded to *10, and R^{a6} to R^{a18} are a hydrogen atom,
provided that two adjacent ones selected from R^{a1} to R^{a5} which are not a single bond bonded to *10 and R^{a6} to R^{a18} may be bonded to each other to form a benzene ring, or may not be bonded to each other and therefore may not form a ring,
Ar^{a1} is a substituted or unsubstituted aryl group having 6 to 30 ring carbon atoms, a substituted or unsubstituted aromatic heterocyclic group having 5 to 30 ring atoms, or a triarylsilyl group having a substituted or unsubstituted aryl group having 6 to 30 ring carbon atoms,
one selected from R^{a21} to R^{a24} is a single bond bonded to *11, and *R^{a21} to R^{a24} which are not a single bond bonded to *11, and R^{a25} to R^{a28} are each independently a hydrogen atom, a substituted or unsubstituted alkyl group having 1 to 30 carbon atoms, a substituted or unsubstituted aryl group having 6 to 30 ring carbon atoms, or a substituted or unsubstituted aromatic heterocyclic group having 5 to 30 ring atoms,
provided that two adjacent ones selected from R^{a21} to R^{a24} which are not a single bond bonded to *11 and R^{a25} to R^{a28} may be bonded to each other to form a benzene ring, or may not be bonded to each other and therefore may not form a ring,
R^{A} and R^{B} are each independently a substituted or unsubstituted alkyl group having 1 to 30 carbon atoms, a substituted or unsubstituted aryl group having 6 to 30 ring carbon atoms, or a substituted or unsubstituted aromatic heterocyclic group having 5 to 30 ring atoms, and
provided that R^{A} and R^{B} may be bonded to each other to form a substituted or unsubstituted spiro ring with the carbon atom at the 9-position of the fluorene skeleton, or may not be bonded to each other and may not form a spiro ring).

In another embodiment, the present invention provides a compound represented by the following formula (2),

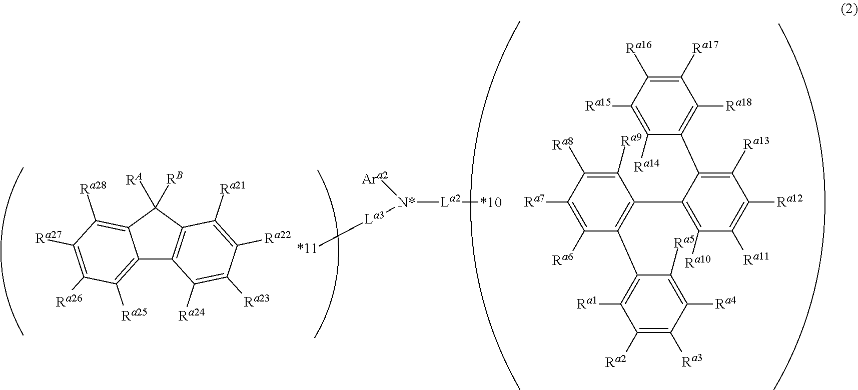

(In the formula, N* is a central nitrogen atom,
L^{a2}, L^{a3}, R^{a1} to R^{a5}, R^{a6} to R^{a18}, R^{a21} to R^{a24}, R^{a25} to R^{a28}, *10, *11, R^{A} and R^{B} are each as defined in formula (1), and
Ar^{a2} is a condensed aryl group having 14 to 30 ring carbon atoms substituted with an unsubstituted alkyl group having 1 to 30 carbon atoms or unsubstituted, an unsubstituted alkyl group having 1 to 30 carbon atoms, or a substituted or unsubstituted condensed aromatic heterocyclic group having 13 to 30 ring atoms, or a substituted or unsubstituted fluorenyl group).

In another embodiment, the present invention provides a compound represented by the following formula (3),

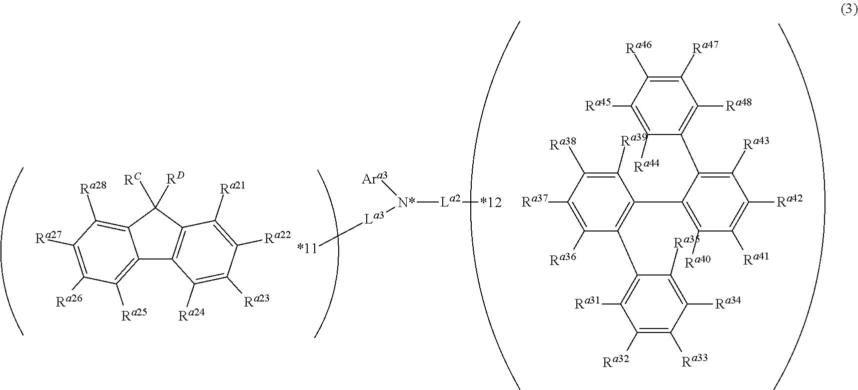

(In the formula, N* is a central nitrogen atom,
L^{a2}, L^{a3}, *11, R^{a21} to R^{a24}, and R^{a25} to R^{a28} are each as defined in formula (1),
one selected from R^{a31} to R^{a48} is a single bond bonded to *12, and R^{a31} to R^{a48} which are not a single bond bonded to *12 are a hydrogen atom,

Provided that two adjacent ones selected from R^{a31} to R^{a48} which are not a single bond bonded to *12 may be bonded to each other to form a benzene ring, or may not be bonded to each other and therefore may not form a ring,
R^{C} and R^{D} are bonded to each other to form a substituted or unsubstituted spiro ring with the carbon atom at the 9-position of the fluorene skeleton, and
Ar^{a3} is a phenyl group or a naphthyl group which may be substituted with a triarylsilyl group having a substituted or unsubstituted aryl group having 6 to 30 ring carbon atoms).

In another embodiment, the present invention provides a material for organic EL device containing a compound represented by any of the above formulas (1) to (3).

Further, in another embodiment, the present invention provides an organic electroluminescent device containing a cathode, an anode, and an organic layer between the cathode and the anode, in which the organic layer contains a light emitting layer, and at least one layer of the organic layers contains the compound represented by the above formulas (1) to (3).

Furthermore, in another embodiment, the present invention provides an electronic device containing the organic electroluminescent device.

### Advantageous Effects of Invention

An organic EL device containing a compound represented by any one of the above formulas (1) to (3) exhibits improved device performance.

### BRIEF DESCRIPTION OF DRAWINGS

FIG. 1 is a schematic diagram of an example of the layer configuration of an organic EL device according to one embodiment of the present invention.

FIG. 2 is a schematic diagram of other examples of the layer configuration of an organic EL device according to one embodiment of the present invention.

FIG. 3 is a schematic diagram of other examples of the layer configuration of an organic EL device according to one embodiment of the present invention.

### DESCRIPTION OF EMBODIMENTS

### Definitions

In the description herein, the hydrogen atom encompasses isotopes thereof having different numbers of neutrons, i.e., a light hydrogen atom (protium), a heavy hydrogen atom (deuterium), and tritium.

In the description herein, the bonding site where the symbol, such as “R”, or “D” representing a deuterium atom is not shown is assumed to have a hydrogen atom, i.e., a protium atom, a deuterium atom, or a tritium atom, bonded thereto.

In the description herein, the number of ring carbon atoms shows the number of carbon atoms among the atoms constituting the ring itself of a compound having a structure including atoms bonded to form a ring (such as a monocyclic compound, a condensed ring compound, a bridged compound, a carbocyclic compound, and a heterocyclic compound). In the case where the ring is substituted by a substituent, the carbon atom contained in the substituent is not included in the number of ring carbon atoms. The same definition is applied to the “number of ring carbon atoms” described hereinafter unless otherwise indicated. For example, a benzene ring has 6 ring carbon atoms, a naphthalene ring has 10 ring carbon atoms, a pyridine ring has 5 ring carbon atoms, and a furan ring has 4 ring carbon atoms. For example, 9,9-diphenylfluorenyl group has 13 ring carbon atoms, and 9,9′-spirobifluorenyl group has 25 ring carbon atoms.

In the case where a benzene ring has, for example, an alkyl group substituted thereon as a substituent, the number of carbon atoms of the alkyl group is not included in the number of ring carbon atoms of the benzene ring. Accordingly, a benzene ring having an alkyl group substituted thereon has 6 ring carbon atoms. In the case where a naphthalene ring has, for example, an alkyl group substituted thereon as a substituent, the number of carbon atoms of the alkyl group is not included in the number of ring carbon atoms of the naphthalene ring. Accordingly, a naphthalene ring having an alkyl group substituted thereon has 10 ring carbon atoms.

In the description herein, the number of ring atoms shows the number of atoms constituting the ring itself of a compound having a structure including atoms bonded to form a ring (such as a monocyclic ring, a condensed ring, and a set of rings) (such as a monocyclic compound, a condensed ring compound, a bridged compound, a carbocyclic compound, and a heterocyclic compound). The atom that does not constitute the ring (such as a hydrogen atom terminating the bond of the atom constituting the ring) and, in the case where the ring is substituted by a substituent, the atom contained in the substituent are not included in the number of ring atoms. The same definition is applied to the “number of ring atoms” described hereinafter unless otherwise indicated. For example, a pyridine ring has 6 ring atoms, a quinazoline ring has 10 ring atoms, and a furan ring has 5 ring atoms. For example, the number of hydrogen atoms bonded to a pyridine ring or atoms constituting a substituent is not included in the number of ring atoms of the pyridine ring. Accordingly, a pyridine ring having a hydrogen atom or a substituent bonded thereto has 6 ring atoms. For example, the number of hydrogen atoms bonded to carbon atoms of a quinazoline ring or atoms constituting a substituent is not included in the number of ring atoms of the quinazoline ring. Accordingly, a quinazoline ring having a hydrogen atom or a substituent bonded thereto has 10 ring atoms.

In the description herein, the expression “having XX to YY carbon atoms” in the expression “substituted or unsubstituted ZZ group having XX to YY carbon atoms” means the number of carbon atoms of the unsubstituted ZZ group, and, in the case where the ZZ group is substituted, the number of carbon atoms of the substituent is not included. Herein, “YY” is larger than “XX”, “XX” represents an integer of 1 or more, and “YY” represents an integer of 2 or more.

In the description herein, the expression “having XX to YY atoms” in the expression “substituted or unsubstituted ZZ group having XX to YY atoms” means the number of atoms of the unsubstituted ZZ group, and, in the case where the ZZ group is substituted, the number of atoms of the substituent is not included. Herein, “YY” is larger than “XX”, “XX” represents an integer of 1 or more, and “YY” represents an integer of 2 or more.

In the description herein, an unsubstituted ZZ group means the case where the “substituted or unsubstituted ZZ group” is an “unsubstituted ZZ group”, and a substituted ZZ group means the case where the “substituted or unsubstituted ZZ group” is a “substituted ZZ group”.

In the description herein, the expression “unsubstituted” in the expression “substituted or unsubstituted ZZ group” means that hydrogen atoms in the ZZ group are not substituted by a substituent. The hydrogen atoms in the “unsubstituted ZZ group” each are a protium atom, a deuterium atom, or a tritium atom.

In the description herein, the expression “substituted” in the expression “substituted or unsubstituted ZZ group” means that one or more hydrogen atom in the ZZ group is substituted by a substituent. The expression “substituted” in the expression “BB group substituted by an AA group” similarly means that one or more hydrogen atom in the BB group is substituted by the AA group.

### Substituents in Description

The substituents described in the description herein will be explained.

In the description herein, the number of ring carbon atoms of the “unsubstituted aryl group” is 6 to 50, preferably 6 to 30, and more preferably 6 to 18, unless otherwise indicated in the description.

In the description herein, the number of ring atoms of the “unsubstituted heterocyclic group” is 5 to 50, preferably 5 to 30, and more preferably 5 to 18, unless otherwise indicated in the description.

In the description herein, the number of carbon atoms of the “unsubstituted alkyl group” is 1 to 50, preferably 1 to 20, and more preferably 1 to 6, unless otherwise indicated in the description.

In the description herein, the number of carbon atoms of the “unsubstituted alkenyl group” is 2 to 50, preferably 2 to 20, and more preferably 2 to 6, unless otherwise indicated in the description.

In the description herein, the number of carbon atoms of the “unsubstituted alkynyl group” is 2 to 50, preferably 2 to 20, and more preferably 2 to 6, unless otherwise indicated in the description.

In the description herein, the number of ring carbon atoms of the “unsubstituted cycloalkyl group” is 3 to 50, preferably 3 to 20, and more preferably 3 to 6, unless otherwise indicated in the description.

In the description herein, the number of ring carbon atoms of the “unsubstituted arylene group” is 6 to 50, preferably 6 to 30, and more preferably 6 to 18, unless otherwise indicated in the description.

In the description herein, the number of ring atoms of the “unsubstituted divalent heterocyclic group” is 5 to 50, preferably 5 to 30, and more preferably 5 to 18, unless otherwise indicated in the description.

In the description herein, the number of carbon atoms of the “unsubstituted alkylene group” is 1 to 50, preferably 1 to 20, and more preferably 1 to 6, unless otherwise indicated in the description.

### Substituted or Unsubstituted Aryl Group

In the description herein, specific examples (set of specific examples G1) of the “substituted or unsubstituted aryl group” include the unsubstituted aryl groups (set of specific examples G1A) and the substituted aryl groups (set of specific examples G1B) shown below. (Herein, the unsubstituted aryl group means the case where the “substituted or unsubstituted aryl group” is an “unsubstituted aryl group”, and the substituted aryl group means the case where the “substituted or unsubstituted aryl group” is a “substituted aryl group”.) In the description herein, the simple expression “aryl group” encompasses both the “unsubstituted aryl group” and the “substituted aryl group”.

The “substituted aryl group” means a group formed by substituting one or more hydrogen atom of the “unsubstituted aryl group” by a substituent. Examples of the “substituted aryl group” include groups formed by one or more hydrogen atom of each of the “unsubstituted aryl groups” in the set of specific examples G1A by a substituent, and the examples of the substituted aryl groups in the set of specific examples G1B. The examples of the “unsubstituted aryl group” and the examples of the “substituted aryl group” enumerated herein are mere examples, and the “substituted aryl group” in the description herein encompasses groups formed by substituting a hydrogen atom bonded to the carbon atom of the aryl group itself of each of the “substituted aryl groups” in the set of specific examples G1B by a substituent, and groups formed by substituting a hydrogen atom of the substituent of each of the “substituted aryl groups” in the set of specific examples G1B by a substituent.

### Unsubstituted Aryl Group (Set of Specific Examples G1A):

a phenyl group,
a p-biphenyl group,
a m-biphenyl group,
an o-biphenyl group,
a p-terphenyl-4-yl group,
a p-terphenyl-3-yl group,
a p-terphenyl-2-yl group,
a m-terphenyl-4-yl group,
a m-terphenyl-3-yl group,
a m-terphenyl-2-yl group,
a m-terphenyl-3′-yl group,
an o-terphenyl-4-yl group,
an o-terphenyl-3-yl group,
an o-terphenyl-2-yl group,
a 1-naphthyl group,
a 2-naphthyl group,
an anthryl group,
a benzanthryl group,
a phenanthryl group,
a benzophenanthryl group, a phenarenyl group,
a pyrenyl group,
a chrysenyl group,
a benzochrysenyl group,
a triphenylenyl group,
a benzotriphenylenyl group,
a tetracenyl group,
a pentacenyl group,
a fluorenyl group,
a 9,9′-spirobifluorenyl group,
a benzofluorenyl group,
a dibenzofluorenyl group,
a fluoranthenyl group,
a benzofluoranthenyl group,
a perylenyl group, and
monovalent aryl groups derived by removing one hydrogen atom from each of the ring structures represented by the following general formulae (TEMP-1) to (TEMP-15):

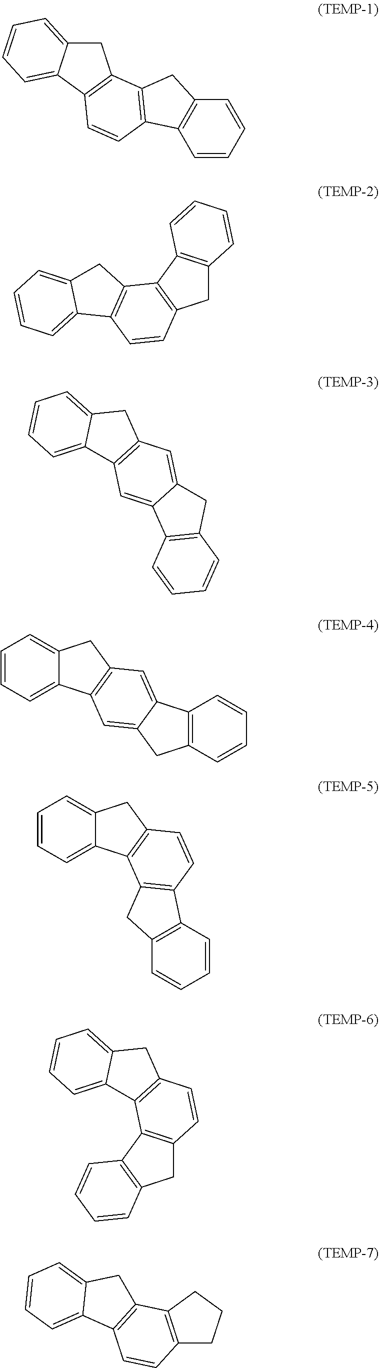

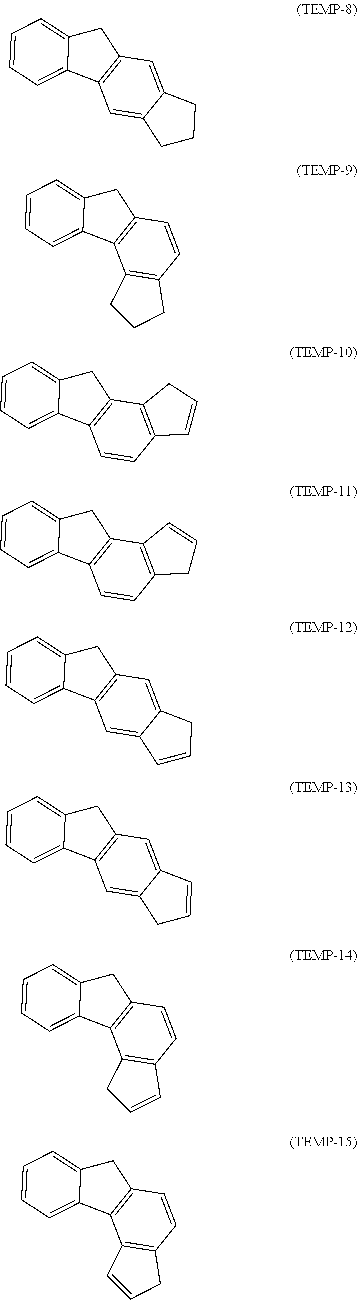

### Substituted Aryl Group (Set of Specific Examples G1B):

an o-tolyl group,
a m-tolyl group,
a p-tolyl group,
a p-xylyl group,
a m-xylyl group,
an o-xylyl group,
a p-isopropylphenyl group,
a m-isopropylphenyl group,
an o-isopropylphenyl group,
a p-t-butylphenyl group,
a m-t-butylphenyl group,
a o-t-butylphenyl group,
a 3,4,5-trimethylphenyl group,
a 9,9-dimethylfluorenyl group,
a 9,9-diphenylfluorenyl group,
a 9,9-bis(4-methylphenyl) fluorenyl group,
a 9,9-bis(4-isopropylphenyl) fluorenyl group,
a 9,9-bis(4-t-butylphenyl) fluorenyl group,
a cyanophenyl group,
a triphenylsilylphenyl group,
a trimethylsilylphenyl group,
a phenylnaphthyl group,
a naphthylphenyl group, and
groups formed by substituting one or more hydrogen atom of each of monovalent aryl groups derived from the ring structures represented by the general formulae (TEMP-1) to (TEMP-15) by a substituent.

### Substituted or Unsubstituted Heterocyclic Group

In the description herein, the “heterocyclic group” means a cyclic group containing at least one hetero atom in the ring atoms. Specific examples of the hetero atom include a nitrogen atom, an oxygen atom, a sulfur atom, a silicon atom, a phosphorus atom, and a boron atom.

In the description herein, the “heterocyclic group” is a monocyclic group or a condensed ring group.

In the description herein, the “heterocyclic group” is an aromatic heterocyclic group or a non-aromatic heterocyclic group.

In the description herein, specific examples (set of specific examples G2) of the “substituted or unsubstituted heterocyclic group” include the unsubstituted heterocyclic groups (set of specific examples G2A) and the substituted heterocyclic groups (set of specific examples G2B) shown below: (Herein, the unsubstituted heterocyclic group means the case where the “substituted or unsubstituted heterocyclic group” is an “unsubstituted heterocyclic group”, and the substituted heterocyclic group means the case where the “substituted or unsubstituted heterocyclic group” is a “substituted heterocyclic group”.) In the description herein, the simple expression “heterocyclic group” encompasses both the “unsubstituted heterocyclic group” and the “substituted heterocyclic group”.

The “substituted heterocyclic group” means a group formed by substituting one or more hydrogen atom of the “unsubstituted heterocyclic group” by a substituent. Specific examples of the “substituted heterocyclic group” include groups formed by substituting a hydrogen atom of each of the “unsubstituted heterocyclic groups” in the set of specific examples G2A by a substituent, and the examples of the substituted heterocyclic groups in the set of specific examples G2B. The examples of the “unsubstituted heterocyclic group” and the examples of the “substituted heterocyclic group” enumerated herein are mere examples, and the “substituted heterocyclic group” in the description herein encompasses groups formed by substituting a hydrogen atom bonded to the ring atom of the heterocyclic group itself of each of the “substituted heterocyclic groups” in the set of specific examples G2B by a substituent, and groups formed by substituting a hydrogen atom of the substituent of each of the “substituted heterocyclic groups” in the set of specific examples G2B by a substituent.

The set of specific examples G2A includes, for example, the unsubstituted heterocyclic group containing a nitrogen atom (set of specific examples G2A1), the unsubstituted heterocyclic group containing an oxygen atom (set of specific examples G2A2), the unsubstituted heterocyclic group containing a sulfur atom (set of specific examples G2A3), and monovalent heterocyclic groups derived by removing one hydrogen atom from each of the ring structures represented by the following general formulae (TEMP-16) to (TEMP-33) (set of specific examples G2A4).

The set of specific examples G2B includes, for example, the substituted heterocyclic groups containing a nitrogen atom (set of specific examples G2B1), the substituted heterocyclic groups containing an oxygen atom (set of specific examples G2B2), the substituted heterocyclic groups containing a sulfur atom (set of specific examples G2B3), and groups formed by substituting one or more hydrogen atom of each of monovalent heterocyclic groups derived from the ring structures represented by the following general formulae (TEMP-16) to (TEMP-33) by a substituent (set of specific examples G2B4).

### Unsubstituted Heterocyclic Group Containing Nitrogen Atom (Set of Specific Examples G2A1):

a pyrrolyl group,
an imidazolyl group,
a pyrazolyl group,
a triazolyl group,
a tetrazolyl group,
an oxazolyl group,
an isoxazolyl group,
an oxadiazolyl group,
a thiazolyl group,
an isothiazolyl group,
a thiadiazolyl group,
a pyridyl group,
a pyridazinyl group,
a pyrimidinyl group,
a pyrazinyl group,
a triazinyl group,
an indolyl group,
an isoindolyl group,
an indolizinyl group,
a quinolizinyl group,
a quinolyl group,
an isoquinolyl group,
a cinnolinyl group,
a phthalazinyl group,
a quinazolinyl group,
a quinoxalinyl group,
a benzimidazolyl group,
an indazolyl group,
a phenanthrolinyl group,
a phenanthridinyl group,
an acridinyl group,
a phenazinyl group,
a carbazolyl group,
a benzocarbazolyl group,
a morpholino group,
a phenoxazinyl group,
a phenothiazinyl group,
an azacarbazolyl group, and a diazacarbazolyl group.

### Unsubstituted Heterocyclic Group Containing Oxygen Atom (Set of Specific Examples G2A2):

a furyl group,
an oxazolyl group,
an isoxazolyl group,
an oxadiazolyl group,
a xanthenyl group,
a benzofuranyl group,
an isobenzofuranyl group,
a dibenzofuranyl group,
a naphthobenzofuranyl group,
a benzoxazolyl group,
a benzisoxazolyl group,
a phenoxazinyl group,
a morpholino group,
a dinaphthofuranyl group,
an azadibenzofuranyl group,
a diazadibenzofuranyl group,
an azanaphthobenzofuranyl group, and
a diazanaphthobenzofuranyl group.

### Unsubstituted Heterocyclic Group Containing Sulfur Atom (Set of Specific Examples G2A3):

a thienyl group,
a thiazolyl group,
an isothiazolyl group,
a thiadiazolyl group,
a benzothiophenyl group (benzothienyl group),
an isobenzothiophenyl group (isobenzothienyl group),
a dibenzothiophenyl group (dibenzothienyl group),
a naphthobenzothiophenyl group (naphthobenzothienyl group),
a benzothiazolyl group,
a benzisothiazolyl group,
a phenothiazinyl group,
a dinaphthothiophenyl group (dinaphthothienyl group),
an azadibenzothiophenyl group (azadibenzothienyl group),
a diazadibenzothiophenyl group (diazadibenzothienyl group),
an azanaphthobenzothiophenyl group (azanaphthobenzothienyl group), and
a diazanaphthobenzothiophenyl group (diazanaphthobenzothienyl group).

Monovalent Heterocyclic Group derived by removing One Hydrogen Atom from Ring Structures represented by General Formulae (TEMP-16) to (TEMP-33) (Set of Specific Examples G2A4)

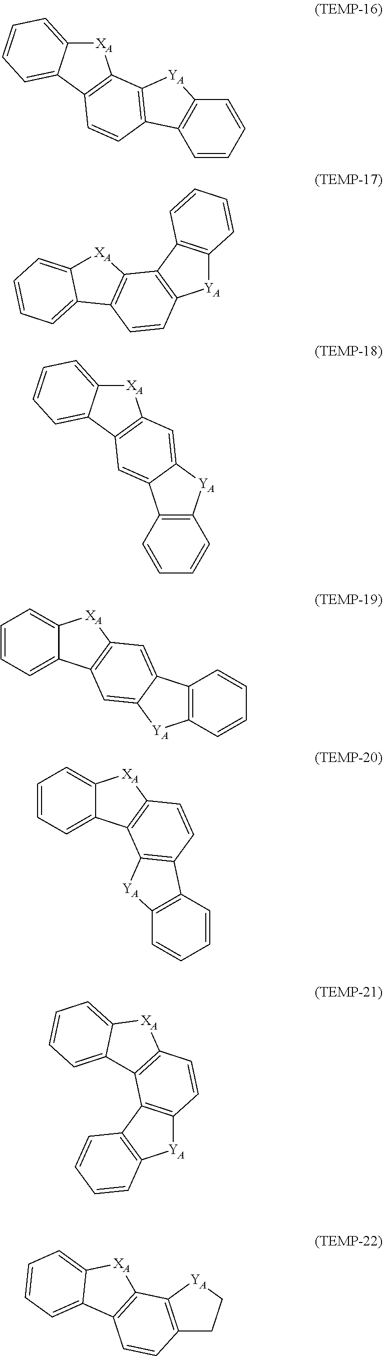

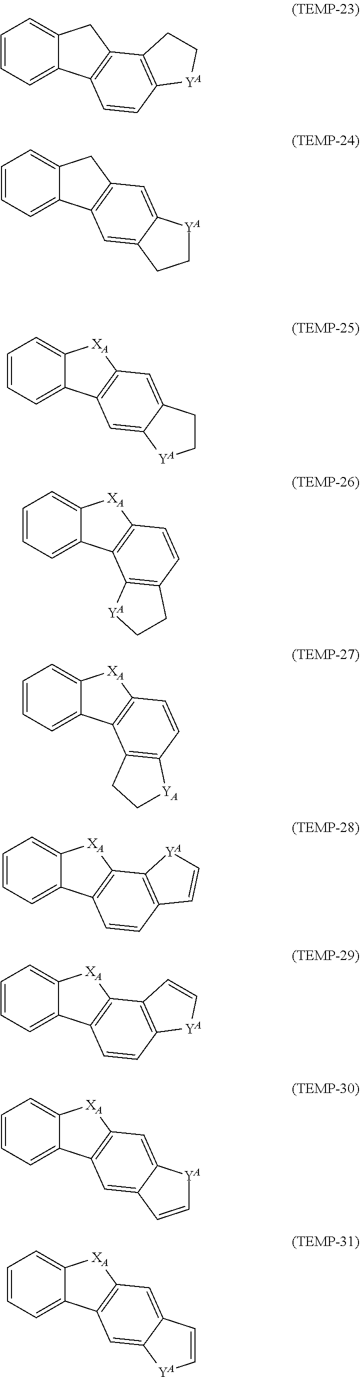

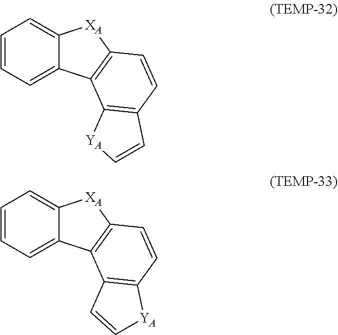

In the general formulae (TEMP-16) to (TEMP-33), X_{A} and Y_{A} each independently represent an oxygen atom, a sulfur atom, NH, or CH₂, provided that at least one of X_{A} and Y_{A} represents an oxygen atom, a sulfur atom, or NH.

In the general formulae (TEMP-16) to (TEMP-33), in the case where at least one of X_{A} and Y_{A} represents NH or CH₂, the monovalent heterocyclic groups derived from the ring structures represented by the general formulae (TEMP-16) to (TEMP-33) include monovalent groups formed by removing one hydrogen atom from the NH or CH₂.

Substituted Heterocyclic Group containing Nitrogen Atom (Set of Specific Examples G2B1):
a (9-phenyl) carbazolyl group,
a (9-biphenylyl) carbazolyl group,
a (9-phenyl)phenylcarbazolyl group,
a (9-naphthyl) carbazolyl group,
a diphenylcarbazol-9-yl group,
a phenylcarbazol-9-yl group,
a methylbenzimidazolyl group,
an ethylbenzimidazolyl group,
a phenyltriazinyl group,
a biphenyltriazinyl group,
a diphenyltriazinyl group,
a phenylquinazolinyl group, and a biphenylquinazolinyl group.

### Substituted Heterocyclic Group Containing Oxygen Atom (Set of Specific Examples G2B2):

a phenyldibenzofuranyl group,
a methyldibenzofuranyl group,
a t-butyldibenzofuranyl group, and
a monovalent residual group of spiro[9H-xanthene-9,9′-[9H]fluorene].

### Substituted Heterocyclic Group Containing Sulfur Atom (Set of Specific Examples G2B3):

a phenyldibenzothiophenyl group,
a methyldibenzothiophenyl group,
a t-butyldibenzothiophenyl group, and
a monovalent residual group of spiro[9H-thioxanthene-9,9′-[9H]fluorene].

Group formed by substituting one or more Hydrogen Atom of Monovalent Heterocyclic Group derived from Ring Structures represented by General Formulae (TEMP-16) to (TEMP-33) by Substituent (Set of Specific Examples G2B4)

The “one or more hydrogen atom of the monovalent heterocyclic group” means one or more hydrogen atom selected from the hydrogen atom bonded to the ring carbon atom of the monovalent heterocyclic group, the hydrogen atom bonded to the nitrogen atom in the case where at least one of X_{A} and Y_{A} represents NH, and the hydrogen atom of the methylene group in the case where one of X_{A} and Y_{A} represents CH₂.

### Substituted or Unsubstituted Alkyl Group

In the description herein, specific examples (set of specific examples G3) of the “substituted or unsubstituted alkyl group” include the unsubstituted alkyl groups (set of specific examples G3A) and the substituted alkyl groups (set of specific examples G3B) shown below. (Herein, the unsubstituted alkyl group means the case where the “substituted or unsubstituted alkyl group” is an “unsubstituted alkyl group”, and the substituted alkyl group means the case where the “substituted or unsubstituted alkyl group” is a “substituted alkyl group”.) In the description herein, the simple expression “alkyl group” encompasses both the “unsubstituted alkyl group” and the “substituted alkyl group”.

The “substituted alkyl group” means a group formed by substituting one or more hydrogen atom of the “unsubstituted alkyl group” by a substituent. Specific examples of the “substituted alkyl group” include groups formed by substituting one or more hydrogen atom of each of the “unsubstituted alkyl groups” (set of specific examples G3A) by a substituent, and the examples of the substituted alkyl groups (set of specific examples G3B). In the description herein, the alkyl group in the “unsubstituted alkyl group” means a chain-like alkyl group. Accordingly, the “unsubstituted alkyl group” encompasses an “unsubstituted linear alkyl group” and an “unsubstituted branched alkyl group”. The examples of the “unsubstituted alkyl group” and the examples of the “substituted alkyl group” enumerated herein are mere examples, and the “substituted alkyl group” in the description herein encompasses groups formed by substituting a hydrogen atom of the alkyl group itself of each of the “substituted alkyl groups” in the set of specific examples G3B by a substituent, and groups formed by substituting a hydrogen atom of the substituent of each of the “substituted alkyl groups” in the set of specific examples G3B by a substituent.

### Unsubstituted Alkyl Group (Set of Specific Examples G3A):

a methyl group,
an ethyl group,
a n-propyl group,
an isopropyl group,
a n-butyl group,
an isobutyl group,
a s-butyl group, and
a t-butyl group.

### Substituted Alkyl Group (Set of Specific Examples G3B):

a heptafluoropropyl group (including isomers),
a pentafluoroethyl group,
a 2,2,2-trifluoroethyl group, and
a trifluoromethyl group.

### Substituted or Unsubstituted Alkenyl Group

In the description herein, specific examples (set of specific examples G4) of the “substituted or unsubstituted alkenyl group” include the unsubstituted alkenyl groups (set of specific examples G4A) and the substituted alkenyl groups (set of specific examples G4B) shown below. (Herein, the unsubstituted alkenyl group means the case where the “substituted or unsubstituted alkenyl group” is an “unsubstituted alkenyl group”, and the substituted alkenyl group means the case where the “substituted or unsubstituted alkenyl group” is a “substituted alkenyl group”.) In the description herein, the simple expression “alkenyl group” encompasses both the “unsubstituted alkenyl group” and the “substituted alkenyl group”.

The “substituted alkenyl group” means a group formed by substituting one or more hydrogen atom of the “unsubstituted alkenyl group” by a substituent. Specific examples of the “substituted alkenyl group” include the “unsubstituted alkenyl groups” (set of specific examples G4A) that each have a substituent, and the examples of the substituted alkenyl groups (set of specific examples G4B). The examples of the “unsubstituted alkenyl group” and the examples of the “substituted alkenyl group” enumerated herein are mere examples, and the “substituted alkenyl group” in the description herein encompasses groups formed by substituting a hydrogen atom of the alkenyl group itself of each of the “substituted alkenyl groups” in the set of specific examples G4B by a substituent, and groups formed by substituting a hydrogen atom of the substituent of each of the “substituted alkenyl groups” in the set of specific examples G4B by a substituent.

### Unsubstituted Alkenyl Group (Set of Specific Examples G4A):

a vinyl group,
an allyl group,
a 1-butenyl group,
a 2-butenyl group, and
a 3-butenyl group.

### Substituted Alkenyl Group (Set of Specific Examples G4B):

a 1,3-butanedienyl group,
a 1-methylvinyl group,
a 1-methylallyl group,
a 1,1-dimethylallyl group,
a 2-methylallyl group, and
a 1,2-dimethylallyl group.

### Substituted or Unsubstituted Alkynyl Group

In the description herein, specific examples (set of specific examples G5) of the “substituted or unsubstituted alkynyl group” include the unsubstituted alkynyl group (set of specific examples G5A) shown below. (Herein, the unsubstituted alkynyl group means the case where the “substituted or unsubstituted alkynyl group” is an “unsubstituted alkynyl group”.) In the description herein, the simple expression “alkynyl group” encompasses both the “unsubstituted alkynyl group” and the “substituted alkynyl group”. The “substituted alkynyl group” means a group formed by substituting one or more hydrogen atom of the “unsubstituted alkynyl group” by a substituent. Specific examples of the “substituted alkenyl group” include groups formed by substituting one or more hydrogen atom of the “unsubstituted alkynyl group” (set of specific examples G5A) by a substituent.

### Unsubstituted Alkynyl Group (Set of Specific Examples G5A):

an ethynyl group.

### Substituted or Unsubstituted Cycloalkyl Group

In the description herein, specific examples (set of specific examples G6) of the “substituted or unsubstituted cycloalkyl group” include the unsubstituted cycloalkyl groups (set of specific examples G6A) and the substituted cycloalkyl group (set of specific examples G6B) shown below: (Herein, the unsubstituted cycloalkyl group means the case where the “substituted or unsubstituted cycloalkyl group” is an “unsubstituted cycloalkyl group”, and the substituted cycloalkyl group means the case where the “substituted or unsubstituted cycloalkyl group” is a “substituted cycloalkyl group”.) In the description herein, the simple expression “cycloalkyl group” encompasses both the “unsubstituted cycloalkyl group” and the “substituted cycloalkyl group”.

The “substituted cycloalkyl group” means a group formed by substituting one or more hydrogen atom of the “unsubstituted cycloalkyl group” by a substituent. Specific examples of the “substituted cycloalkyl group” include groups formed by substituting one or more hydrogen atom of each of the “unsubstituted cycloalkyl groups” (set of specific examples G6A) by a substituent, and the example of the substituted cycloalkyl group (set of specific examples G6B). The examples of the “unsubstituted cycloalkyl group” and the examples of the “substituted cycloalkyl group” enumerated herein are mere examples, and the “substituted cycloalkyl group” in the description herein encompasses groups formed by substituting one or more hydrogen atom bonded to the carbon atoms of the cycloalkyl group itself of the “substituted cycloalkyl group” in the set of specific examples G6B by a substituent, and groups formed by substituting a hydrogen atom of the substituent of the “substituted cycloalkyl group” in the set of specific examples G6B by a substituent.

### Unsubstituted Cycloalkyl Group (Set of Specific Examples G6A):

a cyclopropyl group,
a cyclobutyl group,
a cyclopentyl group,
a cyclohexyl group,
a 1-adamantyl group,
a 2-adamantyl group,
a 1-norbomyl group, and
a 2-norbomyl group.

### Substituted Cycloalkyl Group (Set of Specific Examples G6B):

a 4-methylcyclohexyl group.
   Group Represented by —Si(R₉₀₁)(R₉₀₂)(R₉₀₃)

In the description herein, specific examples (set of specific examples G7) of the group represented by —Si(R₉₀₁)(R₉₀₂)(R₉₀₃) include:
—Si(G1)(G1)(G1),
—Si(G1)(G2)(G2),
—Si(G1)(G1)(G2),
—Si(G2)(G2)(G2),
—Si(G3)(G3)(G3), and
—Si(G6)(G6)(G6). Herein,
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.

Plural groups represented by G1 in —Si(G1)(G1)(G1) are the same as or different from each other.

Plural groups represented by G2 in —Si(G1)(G2)(G2) are the same as or different from each other.

Plural groups represented by G1 in —Si(G1)(G1)(G2) are the same as or different from each other.

Plural groups represented by G2 in —Si(G2)(G2)(G2) are the same as or different from each other.

Plural groups represented by G3 in —Si(G3)(G3)(G3) are the same as or different from each other.

Plural groups represented by G6 in —Si(G6)(G6)(G6) are the same as or different from each other.

### Group Represented by —O—(R₉₀₄)

In the description herein, specific examples (set of specific examples G8) of the group represented by —O—(R₉₀₄) include:
—O(G1),
—O(G2),
—O(G3), and
—O(G6).

Herein,
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.

### Group Represented by —S—(R₉₀₅)

In the description herein, specific examples (set of specific examples G9) of the group represented by —S—(R₉₀₅) include:
—S(G1),
—S(G2),
—S(G3), and
—S(G6).

Herein,
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.
   Group Represented by —N(R₉₀₆)(R₉₀₇)

In the description herein, specific examples (set of specific examples G10) of the group represented by —N(R₉₀₆)(R₉₀₇) include:
—N(G1)(G2),
—N(G2)(G2),
—N(G1)(G2),
—N(G3)(G3), and
—N(G6)(G6).

Herein,
G1 represents the “substituted or unsubstituted aryl group” described in the set of specific examples G1,
G2 represents the “substituted or unsubstituted heterocyclic group” described in the set of specific examples G2,
G3 represents the “substituted or unsubstituted alkyl group” described in the set of specific examples G3, and
G6 represents the “substituted or unsubstituted cycloalkyl group” described in the set of specific examples G6.

Plural groups represented by G1 in —N(G1)(G1) are the same as or different from each other.

Plural groups represented by G2 in —N(G2)(G2) are the same as or different from each other.

Plural groups represented by G3 in —N(G3)(G3) are the same as or different from each other.

Plural groups represented by G6 in —N(G6)(G6) are the same as or different from each other.

### Halogen Atom

In the description herein, specific examples (set of specific examples G11) of the “halogen atom” include a fluorine atom, a chlorine atom, a bromine atom, and an iodine atom.

### Substituted or Unsubstituted Fluoroalkyl Group

In the description herein, the “substituted or unsubstituted fluoroalkyl group” means a group formed by substituting at least one hydrogen atom bonded to the carbon atom constituting the alkyl group in the “substituted or unsubstituted alkyl group” by a fluorine atom, and encompasses a group formed by substituting all the hydrogen atoms bonded to the carbon atoms constituting the alkyl group in the “substituted or unsubstituted alkyl group” by fluorine atoms (i.e., a perfluoroalkyl group). The number of carbon atoms of the “unsubstituted fluoroalkyl group” is 1 to 50, preferably 1 to 30, and more preferably 1 to 18, unless otherwise indicated in the description. The “substituted fluoroalkyl group” means a group formed by substituting one or more hydrogen atom of the “fluoroalkyl group” by a substituent. In the description herein, the “substituted fluoroalkyl group” encompasses a group formed by substituting one or more hydrogen atom bonded to the carbon atom of the alkyl chain in the “substituted fluoroalkyl group” by a substituent, and a group formed by substituting one or more hydrogen atom of the substituent in the “substituted fluoroalkyl group” by a substituent. Specific examples of the “unsubstituted fluoroalkyl group” include examples of groups formed by substituting one or more hydrogen atom in each of the “alkyl group” (set of specific examples G3) by a fluorine atom.

### Substituted or Unsubstituted Haloalkyl Group

In the description herein, the “substituted or unsubstituted haloalkyl group” means a group formed by substituting at least one hydrogen atom bonded to the carbon atom constituting the alkyl group in the “substituted or unsubstituted alkyl group” by a halogen atom, and encompasses a group formed by substituting all the hydrogen atoms bonded to the carbon atoms constituting the alkyl group in the “substituted or unsubstituted alkyl group” by halogen atoms. The number of carbon atoms of the “unsubstituted haloalkyl group” is 1 to 50, preferably 1 to 30, and more preferably 1 to 18, unless otherwise indicated in the description. The “substituted haloalkyl group” means a group formed by substituting one or more hydrogen atom of the “haloalkyl group” by a substituent. In the description herein, the “substituted haloalkyl group” encompasses a group formed by substituting one or more hydrogen atom bonded to the carbon atom of the alkyl chain in the “substituted haloalkyl group” by a substituent, and a group formed by substituting one or more hydrogen atom of the substituent in the “substituted haloalkyl group” by a substituent. Specific examples of the “unsubstituted haloalkyl group” include examples of groups formed by substituting one or more hydrogen atom in each of the “alkyl group” (set of specific examples G3) by a halogen atom. A haloalkyl group may be referred to as a halogenated alkyl group in some cases.

### Substituted or Unsubstituted Alkoxy Group

In the description herein, specific examples of the “substituted or unsubstituted alkoxy group” include a group represented by —O(G3), wherein G3 represents the “substituted or unsubstituted alkyl group” described in the set of specific examples G3. The number of carbon atoms of the “unsubstituted alkoxy group” is 1 to 50, preferably 1 to 30, and more preferably 1 to 18, unless otherwise indicated in the description.

### Substituted or Unsubstituted Alkylthio Group

In the description herein, specific examples of the “substituted or unsubstituted alkylthio group” include a group represented by —S(G3), wherein G3 represents the “substituted or unsubstituted alkyl group” described in the set of specific examples G3. The number of carbon atoms of the “unsubstituted alkylthio group” is 1 to 50, preferably 1 to 30, and more preferably 1 to 18, unless otherwise indicated in the description.

### Substituted or Unsubstituted Aryloxy Group

In the description herein, specific examples of the “substituted or unsubstituted aryloxy group” include a group represented by —O(G1), wherein G1 represents the “substituted or unsubstituted aryl group” described in the set of specific examples G1. The number of ring carbon atoms of the “unsubstituted aryloxy group” is 6 to 50, preferably 6 to 30, and more preferably 6 to 18, unless otherwise indicated in the description.

### Substituted or Unsubstituted Arylthio Group

In the description herein, specific examples of the “substituted or unsubstituted arylthio group” include a group represented by —S(G1), wherein G1 represents the “substituted or unsubstituted aryl group” described in the set of specific examples G1. The number of ring carbon atoms of the “unsubstituted arylthio group” is 6 to 50, preferably 6 to 30, and more preferably 6 to 18, unless otherwise indicated in the description.

### Substituted or Unsubstituted Trialkylsilyl Group

In the description herein, specific examples of the “trialkylsilyl group” include a group represented by —Si(G3)(G3)(G3), wherein G3 represents the “substituted or unsubstituted alkyl group” described in the set of specific examples G3. Plural groups represented by G3 in —Si(G3)(G3)(G3) are the same as or different from each other. The number of carbon atoms of each of alkyl groups of the “substituted or unsubstituted trialkylsilyl group” is 1 to 50, preferably 1 to 20, and more preferably 1 to 6, unless otherwise indicated in the description.

### Substituted or Unsubstituted Aralkyl Group

In the description herein, specific examples of the “substituted or unsubstituted aralkyl group” include a group represented by -(G3)-(G1), wherein G3 represents the “substituted or unsubstituted alkyl group” described in the set of specific examples G3, and G1 represents the “substituted or unsubstituted aryl group” described in the set of specific examples G1. Accordingly, the “aralkyl group” is a group formed by substituting a hydrogen atom of an “alkyl group” by an “aryl group” as a substituent, and is one embodiment of the “substituted alkyl group”. The “unsubstituted aralkyl group” is an “unsubstituted alkyl group” that is substituted by an “unsubstituted aryl group”, and the number of carbon atoms of the “unsubstituted aralkyl group” is 7 to 50, preferably 7 to 30, and more preferably 7 to 18, unless otherwise indicated in the description.

Specific examples of the “substituted or unsubstituted aralkyl group” include a benzyl group, a 1-phenylethyl group, a 2-phenylethyl group, a 1-phenylisopropyl group, a 2-phenylisopropyl group, a phenyl-t-butyl group, an α-naphthylmethyl group, a 1-α-naphthylethyl group, a 2-α-naphthylethyl group, a 1-α-naphthylisopropyl group, a 2-α-naphthylisopropyl group, a β-naphthylmethyl group, a 1-β-naphthylethyl group, a 2-β-naphthylethyl group, a 1-β-naphthylisopropyl group, and a 2-β-naphthylisopropyl group.

In the description herein, the substituted or unsubstituted aryl group is preferably a phenyl group, a p-biphenyl group, a m-biphenyl group, an o-biphenyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, an o-terphenyl-4-yl group, an o-terphenyl-3-yl group, an o-terphenyl-2-yl group, a 1-naphthyl group, a 2-naphthyl group, an anthryl group, a phenanthryl group, a pyrenyl group, a chrysenyl group, a triphenylenyl group, a fluorenyl group, a 9,9′-spirobifluorenyl group, a 9,9-dimethylfluorenyl group, a 9,9-diphenylfluorenyl group, and the like, unless otherwise indicated in the description.

In the description herein, the substituted or unsubstituted heterocyclic group is preferably a pyridyl group, a pyrimidinyl group, a triazinyl group, a quinolyl group, an isoquinolyl group, a quinazolinyl group, a benzimidazolyl group, a phenanthrolinyl group, a carbazolyl group (e.g., a 1-carbazolyl, group, a 2-carbazolyl, group, a 3-carbazolyl, group, a 4-carbazolyl, group, or a 9-carbazolyl, group), a benzocarbazolyl group, an azacarbazolyl group, a diazacarbazolyl group, a dibenzofuranyl group, a naphthobenzofuranly group, an azadibenzofuranyl group, a diazadibenzofuranyl group, a dibenzothiophenyl group, a naphthobenzothiophenyl group, an azadibenzothiophenyl group, a diazadibenzothiophenyl group, a (9-phenyl) carbazolyl group (e.g., a (9-phenyl) carbazol-1-yl group, a (9-phenyl) carbazol-2-yl group, a (9-phenyl) carbazol-3-yl group, or a (9-phenyl) carbazol-4-yl group), a (9-biphenylyl) carbazolyl group, a (9-phenyl)phenylcarbazolyl group, a diphenylcarbazol-9-yl group, a phenylcarbazol-9-yl group, a phenyltriazinyl group, a biphenylyltriazinyl group, a diphenyltriazinyl group, a phenyldibenzofuranyl group, a phenyldibenzothiophenyl group, and the like, unless otherwise indicated in the description.

In the description herein, the carbazolyl group is specifically any one of the following groups unless otherwise indicated in the description.

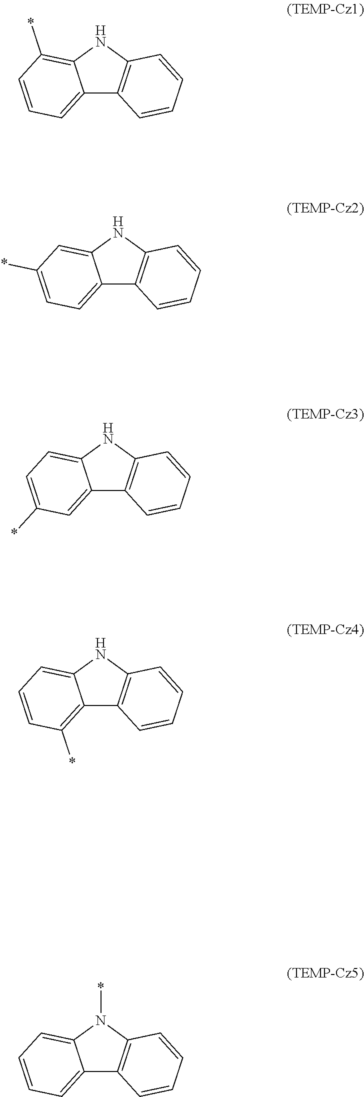 [SMILES: *c1cccc2c1[nH]c1ccccc12 ; *c1ccc2c(c1)[nH]c1ccccc12 ; *c1ccc2[nH]c3ccccc3c2c1 ; *c1cccc2[nH]c3ccccc3c12 ; *n1c2ccccc2c2ccccc21]

In the description herein, the (9-phenyl) carbazolyl group is specifically any one of the following groups unless otherwise indicated in the description.

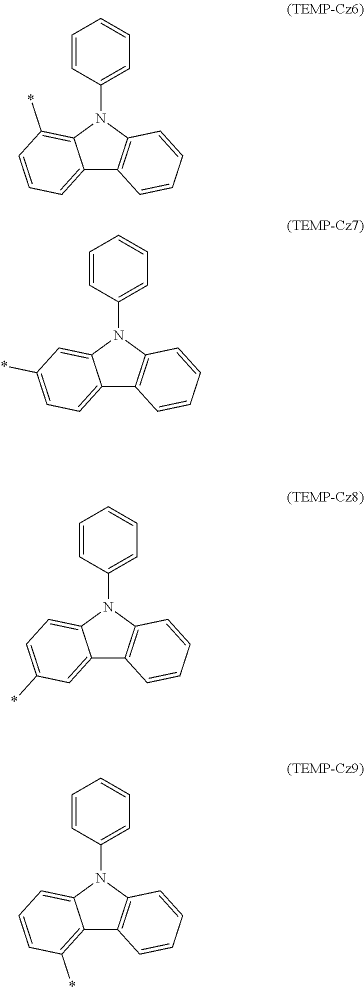

In the general formulae (TEMP-Cz1) to (TEMP-Cz9), * represents a bonding site.

In the description herein, the dibenzofuranyl group and the dibenzothiophenyl group are specifically any one of the following groups unless otherwise indicated in the description.

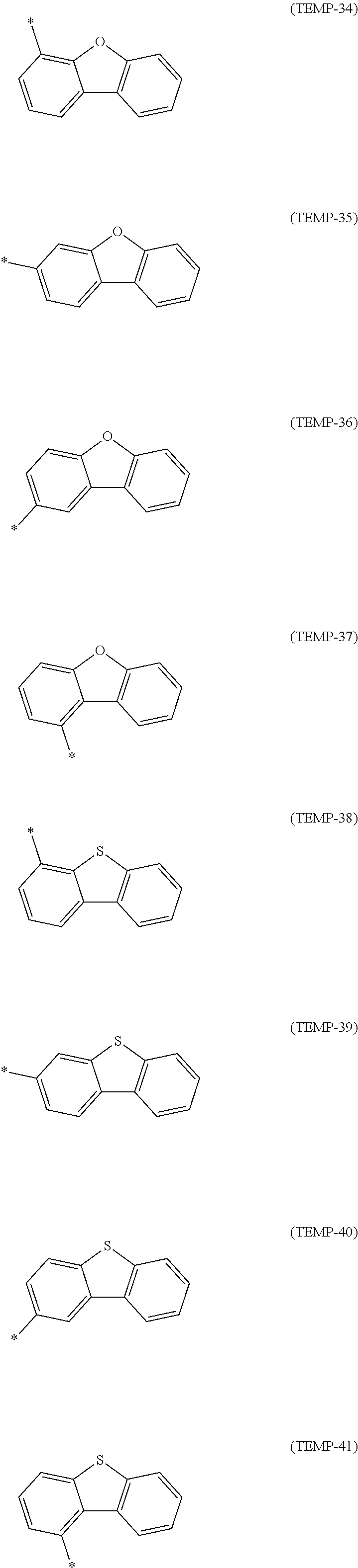

In the general formulae (TEMP-34) to (TEMP-41), * represents a bonding site.

In the description herein, the substituted or unsubstituted alkyl group is preferably a methyl group, an ethyl group, a propyl group, an isopropyl group, a n-butyl group, an isobutyl group, a t-butyl group, or the like unless otherwise indicated in the description.

### Substituted or Unsubstituted Arylene Group

In the description herein, the “substituted or unsubstituted arylene group” is a divalent group derived by removing one hydrogen atom on the aryl ring from the “substituted or unsubstituted aryl group” described above unless otherwise indicated in the description. Specific examples (set of specific examples G12) of the “substituted or unsubstituted arylene group” include divalent groups derived by removing one hydrogen atom on the aryl ring from the “substituted or unsubstituted aryl groups” described in the set of specific examples G1.

### Substituted or Unsubstituted Divalent Heterocyclic Group

In the description herein, the “substituted or unsubstituted divalent heterocyclic group” is a divalent group derived by removing one hydrogen atom on the heterocyclic ring from the “substituted or unsubstituted heterocyclic group” described above unless otherwise indicated in the description. Specific examples (set of specific examples G13) of the “substituted or unsubstituted divalent heterocyclic group” include divalent groups derived by removing one hydrogen atom on the heterocyclic ring from the “substituted or unsubstituted heterocyclic groups” described in the set of specific examples G2.

### Substituted or Unsubstituted Alkylene Group

In the description herein, the “substituted or unsubstituted alkylene group” is a divalent group derived by removing one hydrogen atom on the alkyl chain from the “substituted or unsubstituted alkyl group” described above unless otherwise indicated in the description. Specific examples (set of specific examples G14) of the “substituted or unsubstituted alkylene group” include divalent groups derived by removing one hydrogen atom on the alkyl chain from the “substituted or unsubstituted alkyl groups” described in the set of specific examples G3. In the description herein, the substituted or unsubstituted arylene group is preferably any one of the groups represented by the following general formulae (TEMP-42) to (TEMP-68) unless otherwise indicated in the description.

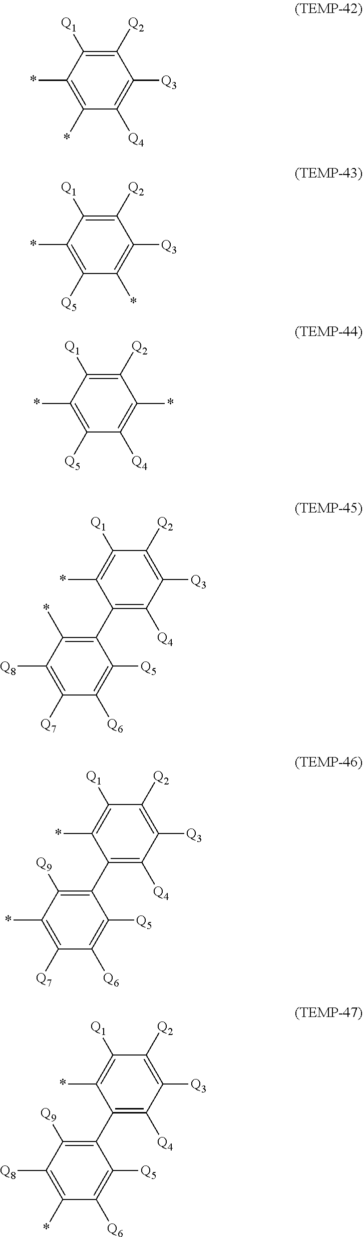

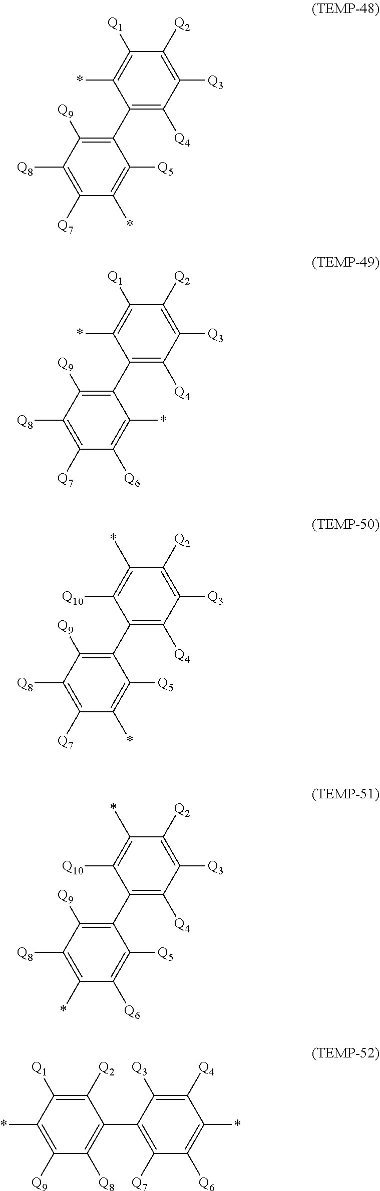

In the general formulae (TEMP-42) to (TEMP-52). Q₁ to Q₁₀ each independently represent a hydrogen atom or a substituent.

In the general formulae (TEMP-42) to (TEMP-52). * represents a bonding site.

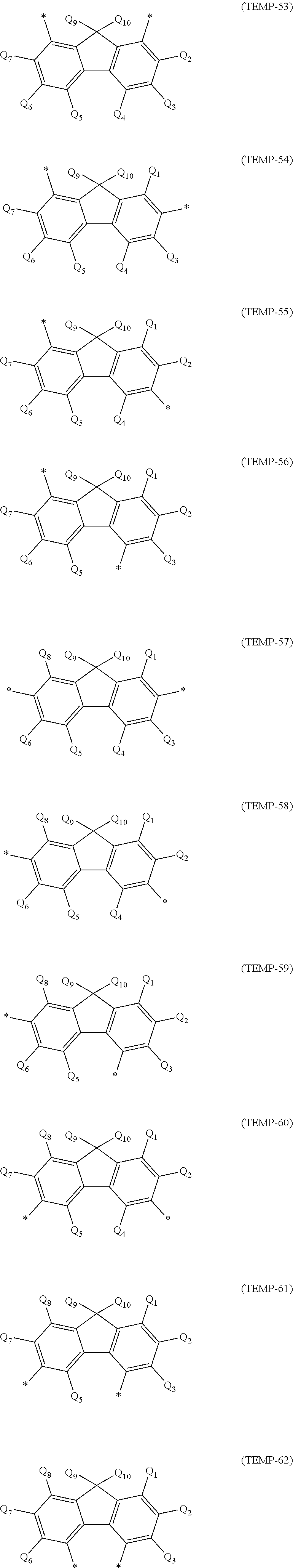

In the general formulae (TEMP-53) to (TEMP-62). Q₁ to Q₁₀ each independently represent a hydrogen atom or a substituent.

The formulae Q₉ and Q₁₀ may be bonded to each other to form a ring via a single bond.

In the general formulae (TEMP-53) to (TEMP-62). * represents a bonding site.

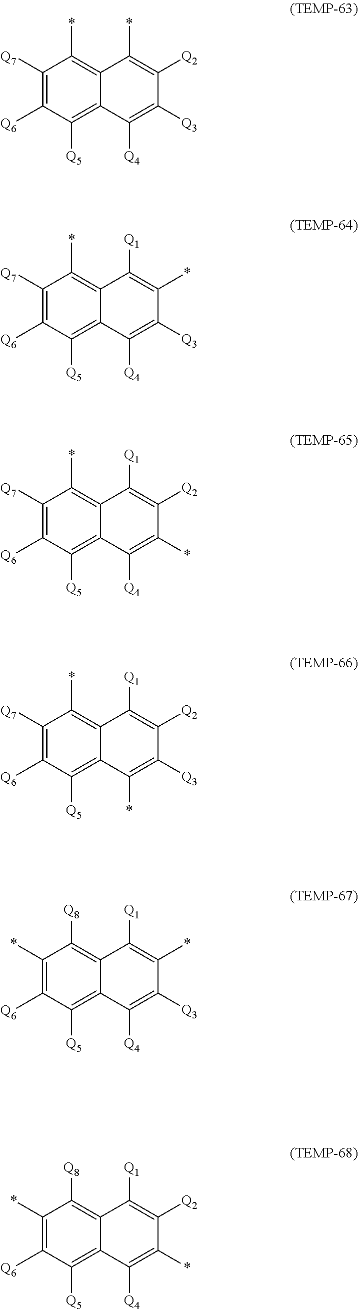

In the general formulae (TEMP-63) to (TEMP-68), Q₁ to Q₈ each independently represent a hydrogen atom or a substituent.

In the general formulae (TEMP-63) to (TEMP-68), * represents a bonding site.

In the description herein, the substituted or unsubstituted divalent heterocyclic group is preferably the groups represented by the following general formulae (TEMP-69) to (TEMP-102) unless otherwise indicated in the description.

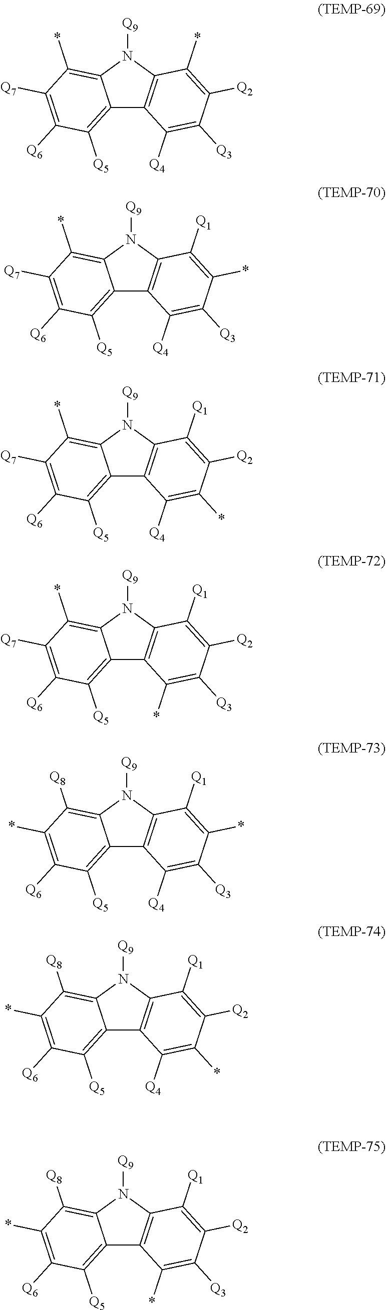

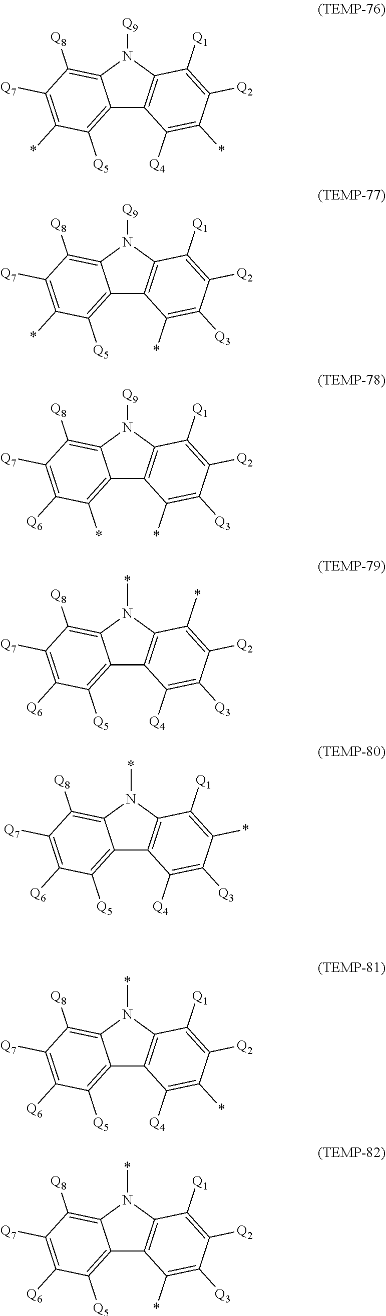

In the general formulae (TEMP-69) to (TEMP-82). Q₁ to Q₉ each independently represent a hydrogen atom or a substituent.

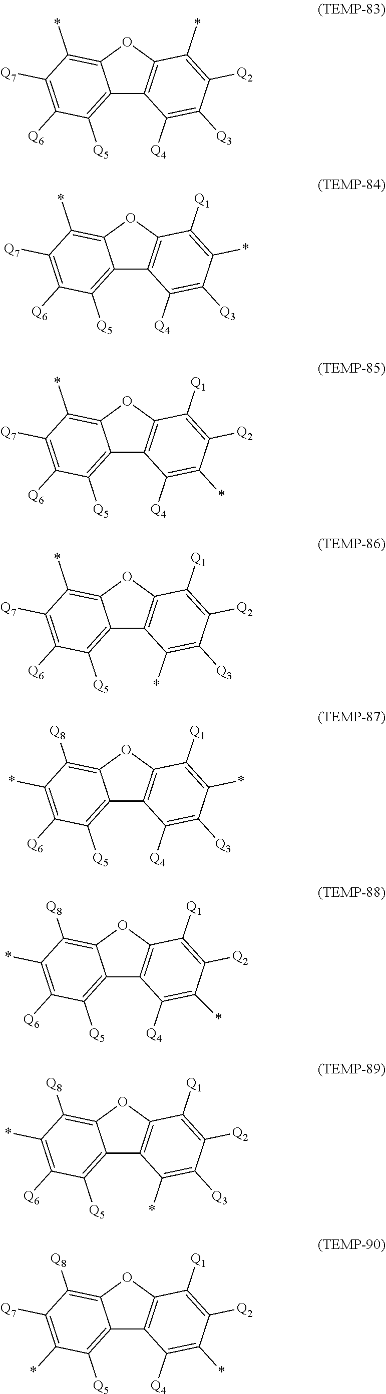

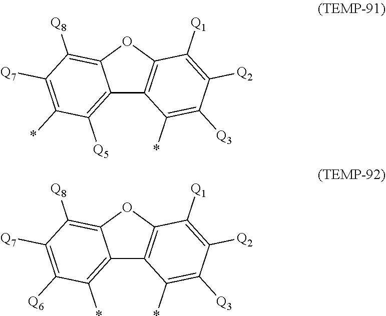

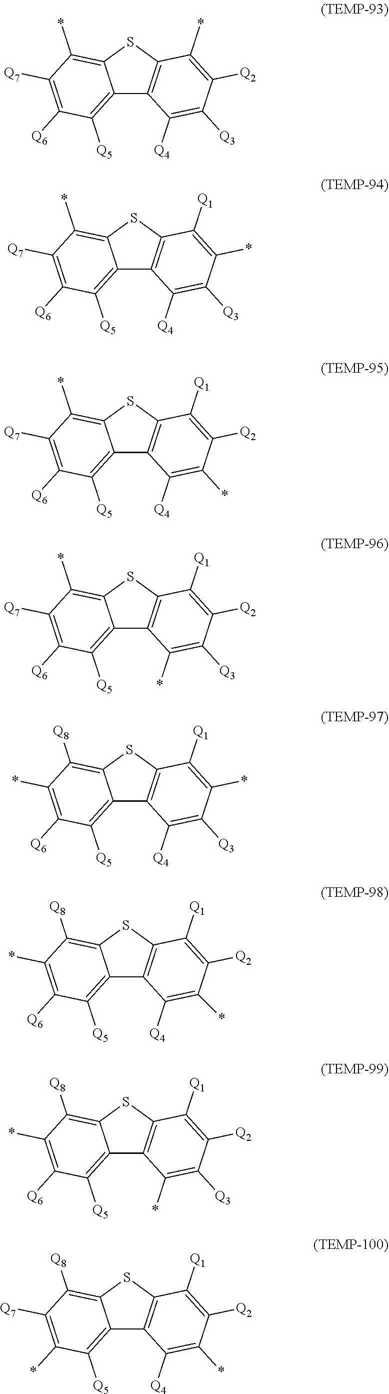

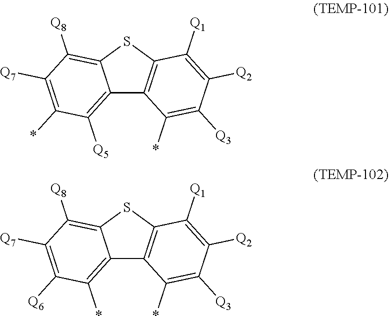

In the general formulae (TEMP-83) to (TEMP-102), Q₁ to Q₈ each independently represent a hydrogen atom or a substituent.

The above are the explanation of the “substituents in the description herein”.

### Case Forming Ring by Bonding

In the description herein, the case where “one or more combinations of combinations each including adjacent two or more each are bonded to each other to form a substituted or unsubstituted monocyclic ring, or each are bonded to each other to form a substituted or unsubstituted condensed ring, or each are not bonded to each other” means a case where “one or more combinations of combinations each including adjacent two or more each are bonded to each other to form a substituted or unsubstituted monocyclic ring”, a case where “one or more combinations of combinations each including adjacent two or more each are bonded to each other to form a substituted or unsubstituted condensed ring”, and a case where “one or more combinations of combinations each including adjacent two or more each are not bonded to each other”.

In the description herein, the case where “one or more combinations of combinations each including adjacent two or more each are bonded to each other to form a substituted or unsubstituted monocyclic ring” and the case where “one or more combinations of combinations each including adjacent two or more each are bonded to each other to form a substituted or unsubstituted condensed ring” (which may be hereinafter collectively referred to as a “case forming a ring by bonding”) will be explained below. The cases will be explained for the anthracene compound represented by the following general formula (TEMP-103) having an anthracene core skeleton as an example.

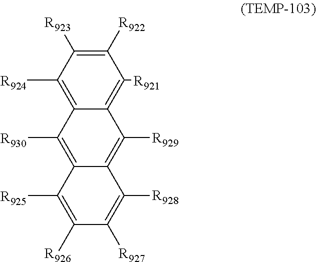

For example, in the case where “one or more combinations of combinations each including adjacent two or more each are bonded to each other to form a ring” among R₉₂₁ to R₉₃₀, the combinations each including adjacent two as one combination include a combination of R₉₂₁ and R₉₂₂, a combination of R₉₂₂ and R₉₂₃, a combination of R₉₂₃ and R₉₂₄, a combination of R₉₂₄ and R₉₃₀, a combination of R₉₃₀ and R₉₂₅, a combination of R₉₂₅ and R₉₂₆, a combination of R₉₂₆ and R₉₂₇, a combination of R₉₂₇ and R₉₂₈, a combination of R₉₂₈ and R₉₂₉, and a combination of R₉₂₉ and R₉₂₁.

The “one or more combinations” mean that two or more combinations each including adjacent two or more may form rings simultaneously. For example, in the case where R₉₂₁ and R₉₂₂ are bonded to each other to form a ring Q_{A}, and simultaneously R₉₂₅ and R₉₂₆ are bonded to each other to form a ring Q_{B}, the anthracene compound represented by the general formula (TEMP-103) is represented by the following general formula (TEMP-104).

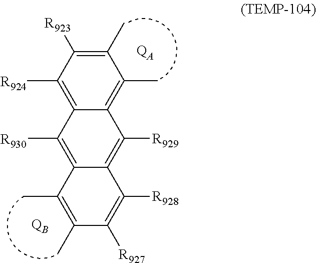

The case where the “combination including adjacent two or more forms rings” encompasses not only the case where adjacent two included in the combination are bonded as in the aforementioned example, but also the case where adjacent three or more included in the combination are bonded. For example, this case means that R₉₂₁ and R₉₂₂ are bonded to each other to form a ring Q_{A}, R₉₂₂ and R₉₂₃ are bonded to each other to form a ring Q_{C}, and adjacent three (R₉₂₁, R₉₂₂, and R₉₂₃) included in the combination are bonded to each other to form rings, which are condensed to the anthracene core skeleton, and in this case, the anthracene compound represented by the general formula (TEMP-103) is represented by the following general formula (TEMP-105). In the following general formula (TEMP-105), the ring Q_{A} and the ring Q_{C} share R₉₂₂.

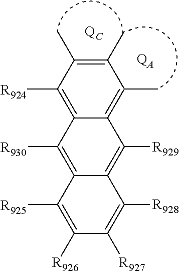

The formed “monocyclic ring” or “condensed ring” may be a saturated ring or an unsaturated ring in terms of structure of the formed ring itself. In the case where the “one combination including adjacent two” forms a “monocyclic ring” or a “condensed ring”, the “monocyclic ring” or the “condensed ring” may form a saturated ring or an unsaturated ring. For example, the ring Q_{A} and the ring Q_{B} formed in the general formula (TEMP-104) each are a “monocyclic ring” or a “condensed ring”. The ring Q_{A} and the ring Q_{C} formed in the general formula (TEMP-105) each are a “condensed ring”. The ring Q_{A} and the ring Q_{C} in the general formula (TEMP-105) form a condensed ring through condensation of the ring Q_{A} and the ring Q_{C}. In the case where the ring Q_{A} in the general formula (TEMP-104) is a benzene ring, the ring Q_{A} is a monocyclic ring. In the case where the ring Q_{A} in the general formula (TEMP-104) is a naphthalene ring, the ring Q_{A} is a condensed ring.

The “unsaturated ring” means an aromatic hydrocarbon ring or an aromatic heterocyclic ring. The “saturated ring” means an aliphatic hydrocarbon ring or a non-aromatic heterocyclic ring.

Specific examples of the aromatic hydrocarbon ring include the structures formed by terminating the groups exemplified as the specific examples in the set of specific examples G1 with a hydrogen atom.

Specific examples of the aromatic heterocyclic ring include the structures formed by terminating the aromatic heterocyclic groups exemplified as the specific examples in the set of specific examples G2 with a hydrogen atom.

Specific examples of the aliphatic hydrocarbon ring include the structures formed by terminating the groups exemplified as the specific examples in the set of specific examples G6 with a hydrogen atom.

The expression “to form a ring” means that the ring is formed only with the plural atoms of the core structure or with the plural atoms of the core structure and one or more arbitrary element. For example, the ring Q_{A} formed by bonding R₉₂₁ and R₉₂₂ each other shown in the general formula (TEMP-104) means a ring formed with the carbon atom of the anthracene skeleton bonded to R₉₂₁, the carbon atom of the anthracene skeleton bonded to R₉₂₂, and one or more arbitrary element. As a specific example, in the case where the ring Q_{A} is formed with R₉₂₁ and R₉₂₂, and in the case where a monocyclic unsaturated ring is formed with the carbon atom of the anthracene skeleton bonded to R₉₂₁, the carbon atom of the anthracene skeleton bonded to R₉₂₂, and four carbon atoms, the ring formed with R₉₂₁ and R₉₂₂ is a benzene ring.

Herein, the “arbitrary element” is preferably at least one kind of an element selected from the group consisting of a carbon element, a nitrogen element, an oxygen element, and a sulfur element, unless otherwise indicated in the description. For the arbitrary element (for example, for a carbon element or a nitrogen element), a bond that does not form a ring may be terminated with a hydrogen atom or the like, and may be substituted by an “arbitrary substituent” described later. In the case where an arbitrary element other than a carbon element is contained, the formed ring is a heterocyclic ring.

The number of the “one or more arbitrary element” constituting the monocyclic ring or the condensed ring is preferably 2 or more and 15 or less, more preferably 3 or more and 12 or less, and further preferably 3 or more and 5 or less, unless otherwise indicated in the description.

What is preferred between the “monocyclic ring” and the “condensed ring” is the “monocyclic ring” unless otherwise indicated in the description.

What is preferred between the “saturated ring” and the “unsaturated ring” is the “unsaturated ring” unless otherwise indicated in the description.

The “monocyclic ring” is preferably a benzene ring unless otherwise indicated in the description.

The “unsaturated ring” is preferably a benzene ring unless otherwise indicated in the description.

In the case where the “one or more combinations of combinations each including adjacent two or more” each are “bonded to each other to form a substituted or unsubstituted monocyclic ring”, or each are “bonded to each other to form a substituted or unsubstituted condensed ring”, it is preferred that the one or more combinations of combinations each including adjacent two or more each are bonded to each other to form a substituted or unsubstituted “unsaturated ring” containing the plural atoms of the core skeleton and 1 or more and 15 or less at least one kind of an element selected from the group consisting of a carbon element, a nitrogen element, an oxygen element, and a sulfur element, unless otherwise indicated in the description.

In the case where the “monocyclic ring” or the “condensed ring” has a substituent, the substituent is, for example, an “arbitrary substituent” described later. In the case where the “monocyclic ring” or the “condensed ring” has a substituent, specific examples of the substituent include the substituents explained in the section “Substituents in Description” described above.

In the case where the “saturated ring” or the “unsaturated ring” has a substituent, the substituent is, for example, an “arbitrary substituent” described later. In the case where the “monocyclic ring” or the “condensed ring” has a substituent, specific examples of the substituent include the substituents explained in the section “Substituents in Description” described above.

The above are the explanation of the case where “one or more combinations of combinations each including adjacent two or more” each are “bonded to each other to form a substituted or unsubstituted monocyclic ring”, and the case where “one or more combinations of combinations each including adjacent two or more” each are “bonded to each other to form a substituted or unsubstituted condensed ring” (i.e., the “case forming a ring by bonding”).

### Substituent for “Substituted or Unsubstituted”

In one embodiment in the description herein, the substituent for the case of “substituted or unsubstituted” (which may be hereinafter referred to as an “arbitrary substituent”) is, for example, a group selected from the group consisting of
an unsubstituted alkyl group having 1 to 50 carbon atoms,
an unsubstituted alkenyl group having 2 to 50 carbon atoms,
an unsubstituted alkynyl group having 2 to 50 carbon atoms,
an unsubstituted cycloalkyl group having 3 to 50 ring carbon atoms,
—Si(R₉₀₁)(R₉₀₂)(R₉₀₃),
—O—(R₉₀₄),
—S—(R₉₀₅),
—N(R₉₀₆) (R₉₀₇),
a halogen atom, a cyano group, a nitro group,
an unsubstituted aryl group having 6 to 50 ring carbon atoms, and
an unsubstituted heterocyclic group having 5 to 50 ring atoms,
wherein R₉₀₁ to R₉₀₇ each independently represent
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or a substituted or unsubstituted heterocyclic group having 5 to 50 ring atoms.

In the case where two or more groups each represented by R₉₀₁ exist, the two or more groups each represented by R₉₀₁ are the same as or different from each other,
in the case where two or more groups each represented by R₉₀₂ exist, the two or more groups each represented by R₉₀₂ are the same as or different from each other,
in the case where two or more groups each represented by R₉₀₃ exist, the two or more groups each represented by R₉₀₃ are the same as or different from each other,
in the case where two or more groups each represented by R₉₀₄ exist, the two or more groups each represented by R₉₀₄ are the same as or different from each other,
in the case where two or more groups each represented by Roos exist, the two or more groups each represented by Roos are the same as or different from each other,
in the case where two or more groups each represented by R₉₀₆ exist, the two or more groups each represented by R₉₀₆ are the same as or different from each other, and
in the case where two or more groups each represented by R₉₀₇ exist, the two or more groups each represented by R₉₀₇ are the same as or different from each other.

In one embodiment, the substituent for the case of “substituted or unsubstituted” may be a group selected from the group consisting of
an alkyl group having 1 to 50 carbon atoms,
an aryl group having 6 to 50 ring carbon atoms, and
a heterocyclic group having 5 to 50 ring atoms.

In one embodiment, the substituent for the case of “substituted or unsubstituted” may be a group selected from the group consisting of
an alkyl group having 1 to 18 carbon atoms,
an aryl group having 6 to 18 ring carbon atoms, and
a heterocyclic group having 5 to 18 ring atoms.

The specific examples of the groups for the arbitrary substituent described above are the specific examples of the substituent described in the section “Substituents in Description” described above.

In the description herein, the arbitrary adjacent substituents may form a “saturated ring” or an “unsaturated ring”, preferably form a substituted or unsubstituted saturated 5-membered ring, a substituted or unsubstituted saturated 6-membered ring, a substituted or unsubstituted unsaturated 5-membered ring, or a substituted or unsubstituted unsaturated 6-membered ring, and more preferably form a benzene ring, unless otherwise indicated.

In the description herein, the arbitrary substituent may further have a substituent unless otherwise indicated in the description. The definition of the substituent that the arbitrary substituent further has may be the same as the arbitrary substituent.

In the description herein, a numerical range shown by “AA to BB” means a range including the numerical value AA as the former of “AA to BB” as the lower limit value and the numerical value BB as the latter of “AA to BB” as the upper limit value.

Hereinafter, the compound of the present invention will be explained.

The compound of the present invention is represented by the above formula (1), (2) or (3). Hereinafter, the symbols in formulas (1) to (3) and the symbols included in the formulas (1) to (3) described later, will be explained. Unless otherwise specified, the same symbols have the same meaning.

The compound of the present invention represented by the formulas (1) to (3) and the formulas included in the formulas (1) to (3) described later may be referred to as “inventive compound”.

The first compound of the present invention (inventive compound (1)) is represented by the following formula (1).

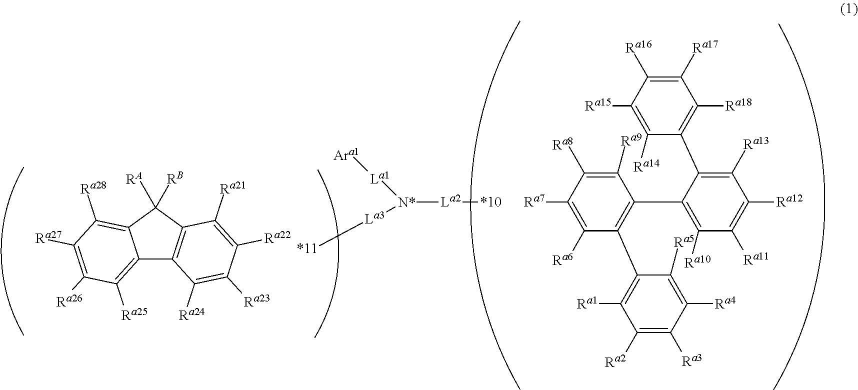

N* is a central nitrogen atom.
L^{a1} is a substituted or unsubstituted phenylene group, a substituted or unsubstituted naphthylene group, or a substituted or unsubstituted biphenylene group. An unsubstituted phenylene group, an unsubstituted naphthylene group, or an unsubstituted biphenylene group are preferred, and an unsubstituted phenylene group is more preferred. No ring is condensed with the phenylene group, the biphenylene group, or the naphthylene group.

The phenylene group is o-phenylene group, m-phenylene group, or p-phenylene group, and is preferably p-phenylene group.

The naphthylene group is preferably 1,4-naphthylene group or 2,6-naphthylene group, more preferably 1,4-naphthylene group.

The biphenylene group is preferably 4,4′-biphenylene group or 3,4′-biphenylene group, more preferably 4,4′-biphenylene group.

The substituent is selected from an unsubstituted alkyl group having 1 to 6 carbon atoms or an unsubstituted aryl group having 6 to 12 ring carbon atoms. The unsubstituted alkyl group having 1 to 6 carbon atoms is preferably a methyl group, an ethyl group, a n-propyl group, an isopropyl group, a n-butyl group, an isobutyl group, a s-butyl group, or a t-butyl group, more preferably a methyl group, an ethyl group, an isopropyl group, or a t-butyl group, even further preferably a methyl group or a t-butyl group. The unsubstituted aryl group having 6 to 12 ring carbon atoms is preferably phenyl group, biphenyl group, or naphthyl group, more preferably phenyl group or naphthyl group, further preferably phenyl group.

L^{a2} and L^{a3} are each independently a single bond, a substituted or unsubstituted phenylene group, a substituted or unsubstituted naphthylene group, or a substituted or unsubstituted biphenylene group, and the substituent is an unsubstituted alkyl group having 1 to 6 carbon atoms or an unsubstituted aryl group having 6 to 12 ring carbon atoms, and no ring is condensed with the phenylene group, the biphenylene group, and the naphthylene group.

The details of the substituted or unsubstituted phenylene group, the substituted or unsubstituted naphthylene group, the substituted or unsubstituted biphenylene group and the substituent are as described for L^{a1}.

In one embodiment of the present invention, L^{a2} is a single bond, and in another embodiment, L^{a2} is a substituted or unsubstituted phenylene group, a substituted or unsubstituted naphthylene group, or a substituted or unsubstituted biphenylene group.

In one embodiment of the present invention, L^{a3} is a single bond, and in another embodiment, L^{a3} is a substituted or unsubstituted phenylene group, a substituted or unsubstituted naphthylene group, or a substituted or unsubstituted biphenylene group.

In one embodiment of the present invention, one or both of the L^{a2} and the L^{a3} is preferably a single bond.

One selected from R^{a1} to R^{a5}, preferably one selected from R^{a1} to R^{a3}, more preferably R^{a1} is a single bond bonded to *10.

R^{a1} to R^{a5} which are not a single bond bonded to *10 and R^{a6} to R^{a18} are a hydrogen atom. Provided that two adjacent ones selected from R^{a1} to R^{a5} which are not a single bond bonded to *10 and R^{a6} to R^{a18} may be bonded to each other to form a benzene ring, or may not be bonded to each other and therefore may not form a ring.

One selected from R^{a21} to R^{a24}, preferably R^{a22}, R^{a23} or R^{a24}, more preferably R^{a22}, is a single bond bonded to *11.

R^{a21} to R^{a24} which are not single bond bonded to *11 and R^{a25} to R^{a28} are each independently a hydrogen atom: a substituted or unsubstituted alkyl group having 1 to 30 carbon atoms, preferably 1 to 18 carbon atoms, more preferably 1 to 6 carbon atoms; a substituted or unsubstituted aryl group having 6 to 30 ring carbon atoms, preferably 6 to 25 carbon atoms, more preferably 6 to 12 carbon atoms; or a substituted or unsubstituted aromatic heterocyclic group having 5 to 30 ring atoms, preferably 5 to 24 ring atoms, more preferably 5 to 13 ring atoms.

The unsubstituted alkyl group having 1 to 30 carbon atoms is, for example, a methyl group, an ethyl group, a n-propyl group, an isopropyl group, a n-butyl group, an isobutyl group, a s-butyl group, a t-butyl group, a pentyl group, a hexyl group, a heptyl group, an octyl group, a nonyl group, a decyl group, an undecyl group or a dodecyl group;
more preferably, a methyl group, an ethyl group, a n-propyl group, an isopropyl group, a n-butyl group, an isobutyl group, a s-butyl group, a t-butyl group, or a pentyl group;
more preferably, a methyl group, an ethyl group, a n-propyl group, an isopropyl group, a n-butyl group, an isobutyl group, a s-butyl group, or a t-butyl group;
further preferably, a methyl group or a t-butyl group.

The unsubstituted aryl group having 6 to 30 ring carbon atoms is, for example, a phenyl group, a biphenylyl group, a terphenylyl group, a biphenylenyl group, a naphthyl group, an anthryl group, a benzanthryl group, a phenanthryl group, a benzophenanthryl group, a phenalenyl group, a picenyl group, a pentaphenyl group, a pyrenyl group, a chrysenyl group, a benzocrysenyl group, a fluorenyl group, a fluoranthenyl group, a perylenyl group, or a triphenylenyl group;
preferably, a phenyl group, a biphenylyl group, a terphenylyl group, or a naphthyl group;
more preferably, a phenyl group, 2-, 3- or 4-biphenylyl group, 2-, 3-, or 4-o-terphenylyl group, 2-, 3-, or 4-m-terphenylyl group, 2-, 3-, or 4-p-terphenyl group, or 1- or 2-naphthyl group;
further preferably, a phenyl group, 2-, 3- or 4-biphenylyl group, or a 1- or 2-naphthyl group; and
particularly preferably a phenyl group.

The substituted aryl group having 6 to 30 ring carbon atoms is preferably, for example, a 9,9-diphenylfluorenyl group, a 9,9-dimethylfluorenyl group, or 9,9-methylphenylfluorenyl group.

The unsubstituted aromatic heterocyclic group having 5 to 30 ring atoms is, for example, a pyrrolyl group, a furyl group, a thienyl group, a pyridyl group, imidazopyridyl group, a pyridazinyl group, a pyrimidinyl group, a pyrazinyl group, triazinyl group, an imidazolyl group, an oxazolyl group, a thiazolyl group, a pyrazolyl group, an isoxazolyl group, an isothiazolyl group, an oxadiazolyl group, a thiadiazolyl group, a triazolyl group, a tetrazolyl group, an indolyl group, an isoindolyl group, an indolizinyl group, a quinolidinyl group, a quinolyl group, an isoquinolyl group, a cinnolyl group, a phthalazinyl group, a quinazolinyl group, a quinoxalinyl group, a benzimidazolyl group, a benzoxazolyl group, a benzothiazolyl group, an indazolyl group, a benzisoxazolyl group, a benzisothiazolyl group, a phenanthridinyl group, an acridinyl group, a phenanthrolinyl group, a phenazinyl group, a phenothiazinyl group, a phenoxazinyl group, a xanthenyl group, a benzofuranyl group, an isobenzofuranyl group, a naphthobenzofuranyl group, a dibenzofuranyl group, a benzothiophenyl group (benzothienyl group, same applies hereinafter), an isobenzothiophenyl group (isobenzothienyl group, same applies hereinafter), a naphthobenzothiophenyl group (naphthobenzothienyl group, same applies hereinafter), a dibenzothiophenyl group (dibenzothienyl group, same applies hereinafter), or a carbazolyl group;

preferably, a benzofuranyl group, an isobenzofuranyl group, a naphthobenzofuranyl group, a dibenzofuranyl group, a benzothiophenyl group, an isobenzothiophenyl group, a naphthobenzothiophenyl group, a dibenzothiophenyl group, a carbazolyl group (9-carbazolyl group, or 1-, 2-, 3-, or 4-carbazolyl group).

Two adjacent ones selected from R^{a21} to R^{a24} which are not a single bond bonded to *11 and R^{a25} to R^{a28} (R^{a21} and R^{a22}, R^{a22} and R^{a23}, R^{a23} and R^{a24}, R^{a25} and R^{a26}, R^{a26} and R^{a27}, R^{a27} and R^{a28}) may be bonded to each other to form a benzene ring, or may not be bonded to each other and therefore may not form a ring.

All of R^{a21} to R^{a24} and R^{a25} to R^{a28} which are not a single bond bonded to *11, may be a hydrogen atom.

R^{A} and R^{B} are each independently a substituted or unsubstituted alkyl group having 1 to 30 carbon atoms, a substituted or unsubstituted aryl group having 6 to 30 ring carbon atoms, or a substituted or unsubstituted aromatic heterocyclic group having 5 to 30 ring atoms. Provided that R^{A} and R^{B} may be bonded to each other to form a substituted or unsubstituted spiro ring with the carbon atom at the 9-position of the fluorene skeleton, or may not be bonded to each other and may not form a spiro ring.

The details of the substituted or unsubstituted alkyl group having 1 to 30 carbon atoms, or the substituted or unsubstituted aryl group having 6 to 30 ring carbon atoms, or the substituted or unsubstituted aromatic heterocyclic group having 5 to 30 ring atoms are each described for R^{a21} to R^{a24} which are not a single bond bonded to *11 and R^{a25} to R^{a28}.

In one embodiment of the present invention, at least one of R^{A} and R^{B} is preferably a substituted or unsubstituted alkyl group having 1 to 30 carbon atoms.

In one embodiment of the present invention, R^{A} and R^{B} are each independently preferably a methyl group or a phenyl group.

In another embodiment of the present invention, R^{A} and R^{B} preferably form a substituted or unsubstituted spiro ring.

The spiro ring is a hydrocarbon ring or a heterocyclic ring, and selected from monocyclic, bridged bicyclo ring, and bridged tricyclo ring. Examples of substituted or unsubstituted spiro rings are shown below; but are not limited thereto. * represents the bonding position of the fluorene skeleton to the benzene ring.

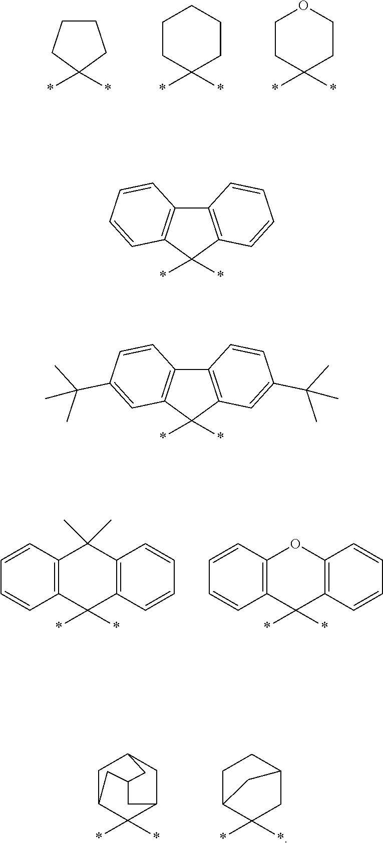

Ar^{a1} is a substituted or unsubstituted aryl group having 6 to 30 ring carbon atoms, a substituted or unsubstituted aromatic heterocyclic group having 5 to 30 ring atoms, or a triarylsilyl group having a substituted or unsubstituted aryl group having 6 to 30 ring carbon atoms. The substituted or unsubstituted aryl group having 6 to 30 ring carbon atoms, or the substituted or unsubstituted aromatic heterocyclic group having 5 to 30 ring atoms are each described for R^{a21} to R^{a24} which are not a single bond bonded to *11 and R^{a25} to R^{a28}. The details of the substituted or unsubstituted aryl group having 6 to 30 ring carbon atoms in the triarylsilyl group are as described for R^{a21} to R^{a24} and R^{a25} to R^{a28}, which are not a single bond bonded to *11. The triarylsilyl group is preferably a triphenylsilyl group.

In one embodiment of the present invention, Ar^{a1} is preferably represented by the following (1a), (1c), (1d), (1e), or (1f), more preferably represented by (1a), (1c), or (1e).

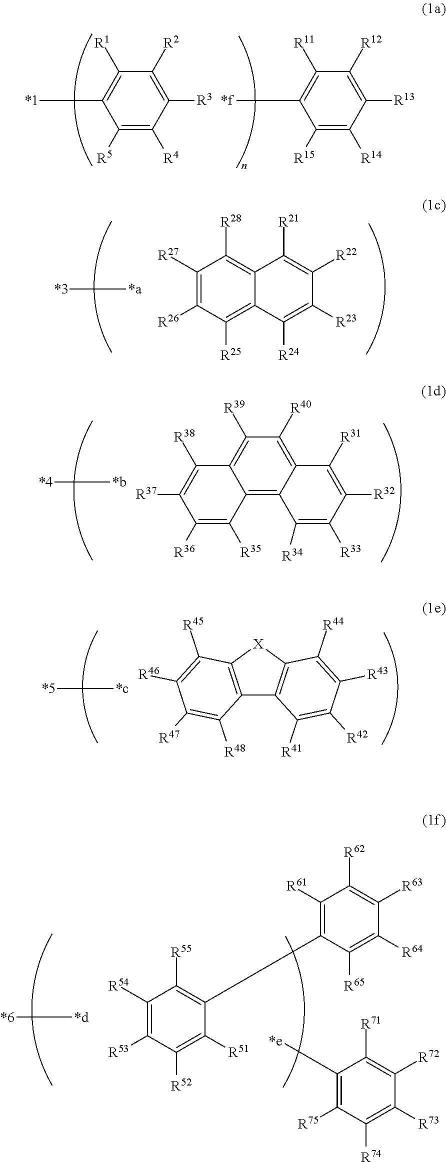

In the formula (1a),

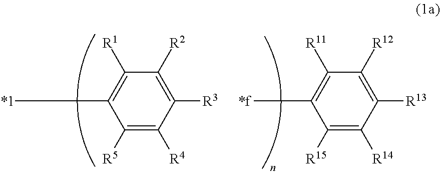

*1 is bonded to L^{a1};
R¹ to R⁵ are each independently a hydrogen atom, a substituted or unsubstituted alkyl group having 1 to 6 carbon atoms, a substituted or unsubstituted aryl group having 6 to 12 ring carbon atoms;
provided that one selected from R¹ to R⁵ is a single bond bonded to *f;
adjacent two ones selected from R¹ to R⁵ that are not a single bond bonded to *f are not bonded to each other and therefore do not form a ring structure;
n is 0 or 1;
R¹¹ and R¹⁵ are each independently a hydrogen atom, a substituted or unsubstituted alkyl group having 1 to 6 carbon atoms, or a substituted or unsubstituted aryl group having 6 to 12 ring carbon atoms; and
two adjacent ones selected from R¹¹ to R¹⁵ are not bonded to each other and therefore do not form a ring structure.

The unsubstituted alkyl group having 1 to 6 carbon atoms is preferably a methyl group, an ethyl group, a n-propyl group, an isopropyl group, a n-butyl group, an isobutyl group, a s-butyl group, or a t-butyl group, more preferably a methyl group, an ethyl group, isopropyl group, or t-butyl group, further preferably a methyl group or a t-butyl group.

The unsubstituted aryl group having 6 to 12 ring carbon atoms is preferably a phenyl group, a biphenyl group, or a naphthyl group, more preferably a phenyl group or a naphthyl group, and further preferably a phenyl group.

The biphenyl group includes an o-biphenyl group, a m-biphenyl group, and a p-biphenyl group, is preferably a m-biphenyl group and a p-biphenyl group, more preferably a p-biphenyl group.

The naphthyl group includes a 1-naphthyl group and a 2-naphthyl group, is preferably 1-naphthyl group.

All of R¹ to R⁵ which are not a single bond bonded to *f of the formula (1a), and R¹¹ to R¹⁵ may be a hydrogen atom.

In the formula (1c),

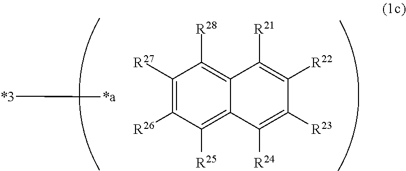

*3 is bonded to L^{a1};
R²¹ to R²⁸ are each independently a hydrogen atom, a substituted or unsubstituted alkyl group having 1 to 6 carbon atoms, or a substituted or unsubstituted aryl group having 6 to 12 ring carbon atoms;
provided that one selected from R²¹ to R²⁸ is a single bond bonded to *a, two adjacent ones selected from R²¹ to R²⁸ which are not single bonds are not bonded to each other and therefore do not form a ring structure.

The details of the unsubstituted alkyl group having 1 to 6 carbon atoms and the aryl group having 6 to 12 ring carbon atoms are the same as the corresponding groups described for formula (1a).

In one embodiment of the present invention, R²¹ is a single bond bonded to *a, in another embodiment, R²² is a single bond bonded to *a.

All of R²¹ to R²⁸ which are not a single bond bonded to *a may be a hydrogen atom.

In the formula (1d),

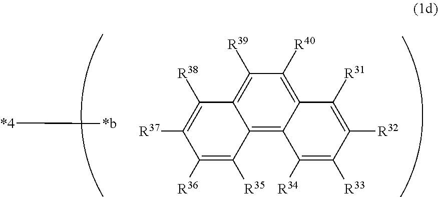

*4 is bonded to L^{a1};
R³¹ to R⁴⁰ are each independently a hydrogen atom, a substituted or unsubstituted alkyl group having 1 to 6 carbon atoms, or a substituted or unsubstituted aryl group having 6 to 12 ring carbon atoms;
provided that one selected from R³¹ to R⁴⁰ is a single bond bonded to *b, two adjacent ones selected from R³¹ to R⁴⁰ which are not single bonds are not bonded to each other and therefore do not form a ring structure.

The details of the unsubstituted alkyl group having 1 to 6 carbon atoms and the unsubstituted aryl group having 6 to 12 ring carbon atoms are the same as the corresponding groups described for formula (1a).

It is preferable that one selected from R³⁷, R³⁸ and R³⁹ is a single bond bonded to *b. In one embodiment of the present invention, R³⁷ is a single bond bonded to *b, in another embodiment R³⁸ is a single bond bonded to *b, and in still another embodiment R³⁹ is a single bond bonded to *b.

All of R³¹ to R⁴⁰ which are not a single bond bonded to *b may be a hydrogen atom.

In the formula (1e),

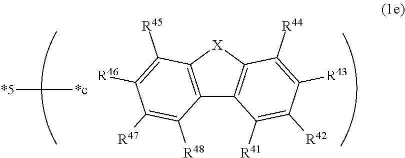

*5 is bonded to L^{a1};
X is an oxygen atom, a sulfur atom, NRa or CRbRc;
Ra is a hydrogen atom, a substituted or unsubstituted alkyl group having 1 to 6 carbon atoms, a substituted or unsubstituted aryl group having 6 to 12 ring carbon atoms, or a substituted or unsubstituted aromatic heterocyclic group having 5 to 13 ring atoms;
Rb and Rc are each independently a substituted or unsubstituted alkyl group having 1 to 6 carbon atoms, or a substituted or unsubstituted aryl group having 6 to 12 ring carbon atoms;
Rb and Rc may be bonded to each other to form a spiro ring structure;
R⁴¹ to R⁴⁸ are each independently a hydrogen atom, a substituted or unsubstituted alkyl group having 1 to 6 carbon atoms, a substituted or unsubstituted aryl group having 6 to 12 ring carbon atoms, or a substituted or unsubstituted aromatic heterocyclic group having 5 to 13 ring atoms;
provided that one selected from R⁴¹ to R⁴⁸ and Ra is a single bond bonded to *c; and
two adjacent ones selected from R⁴¹ to R⁴⁸ which are not single bonds may be bonded to each other to form one or plural substituted or unsubstituted benzene rings.

In one embodiment of the present invention, the formula (1e) is represented by any of the following formulas.

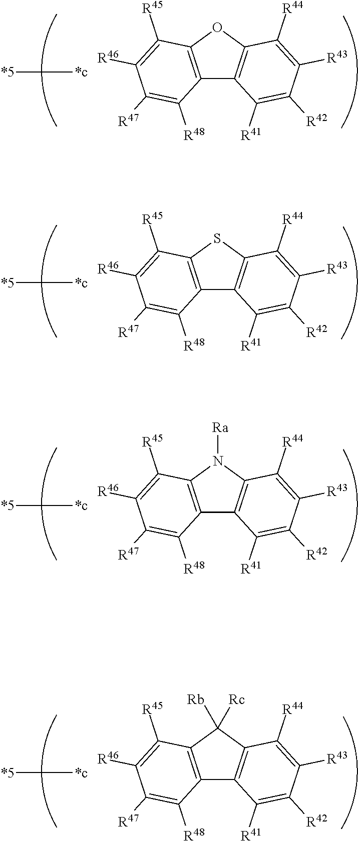

The details of the unsubstituted alkyl group having 1 to 6 carbon atoms and the unsubstituted aryl group having 6 to 12 ring carbon atoms are the same as the corresponding groups described for formula (1a).

The unsubstituted aromatic heterocyclic group having 5 to 13 ring atoms is preferably a pyrrolyl group, a furyl group, a thienyl group, a pyridyl group, a pyrimidinyl group, a triazinyl group, a quinolyl group, an isoquinolyl group, a quinazolinyl group, a benzimidazolyl group, a benzofuranyl group, a benzothiophenyl group (a benzothienyl group), a carbazolyl group, a dibenzofuranyl group, or a dibenzothiophenyl group (a dibenzothienyl group).

Ra is preferably an unsubstituted phenyl group or an unsubstituted naphthyl group (1-naphthyl group or 2-naphthyl group).

It is preferable that both of Rb and Rc are a methyl group or a phenyl group, or that Rb is a phenyl group and Rc is a methyl group.

The details of the spiro ring formed by bonding Rb and Rc to each other are as described for the spiro ring formed by bonding R^{A} and R^{B} to each other in formula (1).

When X is an oxygen atom or a sulfur atom, one selected from R⁴⁵ to R⁴⁸ is preferably a single bond bonded to *c.

When X is NRa, one selected from R⁴⁵ to R⁴⁸ is preferably a single bond bonded to *c.

When X is CRbRc, one selected from R⁴⁵ to R⁴⁸ is preferably a single bond bonded to *c.

All of R⁴¹ to R⁴⁸ which are not a single bond bonded to *c may be a hydrogen atom.

In the formula (1f),

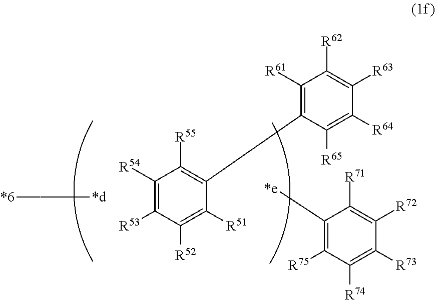

*6 is bonded to L^{a1}.
R⁵¹ to R⁵⁵ are each independently a hydrogen atom, a substituted or unsubstituted alkyl group having 1 to 6 carbon atoms, or unsubstituted phenyl group;
provided that one selected from R⁵¹ to R⁵⁵ is a single bond bonded to *d, and the other selected from R⁵¹ to R⁵⁵ is a single bond bonded to *e;
two adjacent ones selected from R⁵¹ to R⁵⁵ which are neither a single bond bonded to *d nor a single bond bonded to *e are not bonded to each other and therefore do not form a ring structure;
R⁶¹ to R⁶⁵, and R⁷¹ to R⁷⁵ are each independently a hydrogen atom or an unsubstituted alkyl group having 1 to 6 carbon atoms; and two adjacent ones selected from R⁶¹ to R⁶⁵ and R⁷¹ to R⁷⁵ may be bonded to each other to form one or plural substituted or unsubstituted benzene rings.

The details of the unsubstituted alkyl group having 1 to 6 carbon atoms are as described for formula (1a).

In one embodiment of the present invention, two adjacent ones selected from R⁶¹ to R⁶⁵, that is, one or plural adjacent two selected from R⁶¹ and R⁶², R⁶² and R⁶³, R⁶³ and R⁶⁴, and R⁶⁴ and R⁶⁵, are bonded to each other to form one or plural substituted or unsubstituted benzene rings. In another embodiment of the present invention, two adjacent ones selected from R⁶¹ to R⁶⁵ are not bonded to each other and therefore do not form a ring structure.

In one embodiment of the present invention, two adjacent ones selected from R⁷¹ to R⁷⁵, that is, one or plural adjacent two selected from R⁷¹ and R⁷², R⁷² and R⁷³, R⁷³ and R⁷⁴, and R⁷⁴ and R⁷⁵, are bonded to each other to form one or more substituted or unsubstituted benzene rings. In another embodiment of the present invention, two adjacent ones selected from R⁷¹ to R⁷⁵ are not bonded to each other and therefore do not form a ring structure.

All of R⁵¹ to R⁵⁵ which are neither a single bond bonded to *d nor a single bond bonded to *e may be a hydrogen atom, all of R⁶¹ to R⁶⁵ may be a hydrogen atom, or all of R⁷¹ to R⁷⁵ may be a hydrogen atom.

The formula (1f) includes groups represented by the following formulas (1fa) to (1fe), and (1fa), (1fb) or (1fd) are preferred.

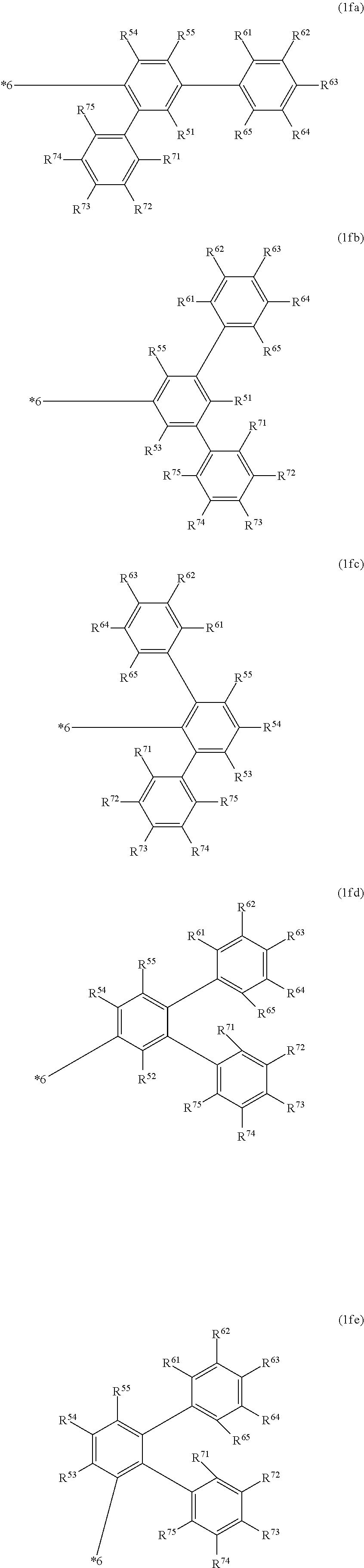

The second compound of the present invention (inventive compound (2)) is represented by the following formula (2).

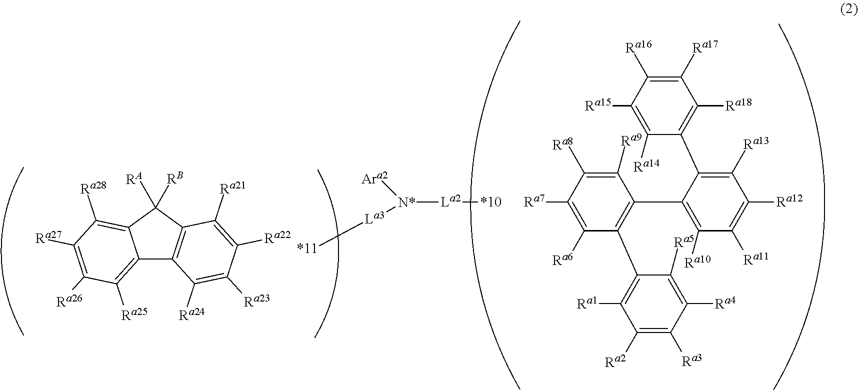

N* is a central nitrogen atom.
L^{a2}, L^{a3}, R^{a1} to R^{a5}, R^{a6} to R^{a18}, R^{a21} to R^{a24}, R^{a25} to R^{a28}, *10, *11, R^{A} and R^{B} are each as defined in formula (1).

Ar^{a2} is a condensed aryl group having 14 to 30 ring carbon atoms substituted with an unsubstituted alkyl group having 1 to 30 carbon atoms or unsubstituted, a substituted or unsubstituted condensed aromatic heterocyclic group having 13 to 30 ring atoms, or a substituted or unsubstituted fluorenyl group.

The unsubstituted alkyl group having 1 to 30 carbon atoms, preferably 1 to 18 carbon atoms, and more preferably 1 to 6 carbon atoms is as described for R^{a21} to R^{a24} which are not a single bond bonded to *11 of the formula (1), and R^{a25} to R^{a28}.

The unsubstituted condensed aryl group having 14 to 30 ring carbon atoms is selected from, for example, a phenanthryl group, an anthryl group, a fluoranthenyl group, a triphenylenyl group, a pyrenyl group, a chrysenyl group, a picenyl group, and a perylenyl group. A phenanthryl group is preferred.

The unsubstituted condensed aromatic heterocyclic group having 13 to 30 ring atoms is selected from, for example, a dibenzofuranyl group, a dibenzothiophenyl group, a carbazolyl group, a phenanthridinyl group, and a phenanthrolinyl group, and a dibenzofuranyl group, a dibenzothiophenyl group, and a carbazolyl group are preferred.

The substituted fluorenyl group is preferably a 9,9-diphenylfluorenyl group, a 9,9-dimethylfluorenyl group, a 9,9-methylphenylfluorenyl group, or a spirofluorenyl group. The details of the spirofluorenyl group are as described for R^{A} and R^{B} in formula (1).

In one embodiment of the present invention, Ar^{a2} is preferably represented by any one of the following formulas (11) to (13).

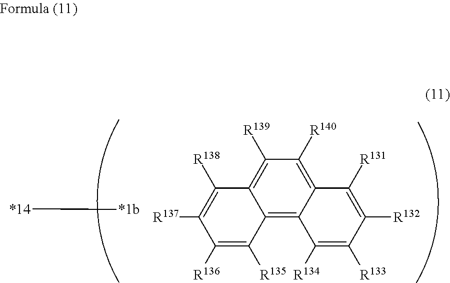

In the formula (11),
*14 is bonded to a central nitrogen atom;
R¹³¹ to R¹⁴⁰ are each independently a hydrogen atom or an unsubstituted alkyl group having 1 to 30 carbon atoms;
provided that one selected from R¹³¹ to R¹⁴⁰ is a single bond bonded to *1b, two adjacent ones selected from R¹³¹ to R¹⁴⁰ which are not single bonds are not bonded to each other and therefore do not form a ring structure.

The details of the unsubstituted alkyl group having 1 to 30 carbon atoms are as described for R^{a21} to R^{a24} which are not a single bond bonded to *11 of the formula (1), and R^{a25} to R^{a28}.

It is preferable that one selected from R¹³⁷, R¹³⁸ and R¹³⁹ is a single bond bonded to *b. In one embodiment of the present invention, R¹³⁷ is a single bond bonded to *1b, in another embodiment R¹³⁸ is a single bond bonded to *b, and in still another embodiment R¹³⁹ is a single bond bonded to *1b.

All of R¹³¹ to R¹⁴⁰ which are not a single bond bonded to *1b may be a hydrogen atom.

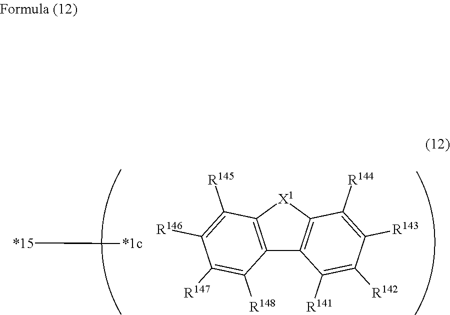

In the formula (12),
*15 is bonded to a central nitrogen atom;
X¹ is an oxygen atom, a sulfur atom, or NRa;
Ra is an unsubstituted phenyl group;
R¹⁴¹ to R¹⁴⁸ are each independently a hydrogen atom or an unsubstituted alkyl group having 1 to 30 carbon atoms;
provided that one selected from R¹⁴¹ to R¹⁴⁸ is a single bond bonded to *1c;
two adjacent ones selected from R¹⁴¹ to R¹⁴⁸ which are not single bonds may be bonded to each other to form one or plural substituted or unsubstituted benzene rings.

The details of the unsubstituted alkyl group having 1 to 30 carbon atoms are as described for R^{a21} to R^{a24} which are not a single bond bonded to *11 of the formula (1), and R^{a25} to R^{a28}.

When X¹ is an oxygen atom or a sulfur atom, one selected from R¹⁴⁵ to R¹⁴⁸ is preferably a single bond bonded to *1c. In one embodiment of the present invention, R¹⁴⁵, in another embodiment R¹⁴⁶, in another embodiment R¹⁴⁷, in another embodiment R¹⁴⁸, is preferably a single bond bonded to *1c.

When X¹ is NRa, one selected from R¹⁴⁵ to R¹⁴⁸ is preferably a single bond bonded to *1c. In one embodiment of the present invention, R¹⁴⁵, in another embodiment R¹⁴⁶, in another embodiment R¹⁴⁷, in another embodiment R¹⁴⁸, is preferably a single bond bonded to *1c.

All of R¹⁴¹ to R¹⁴⁸ which are not a single bond bonded to * 1c may be a hydrogen atom.

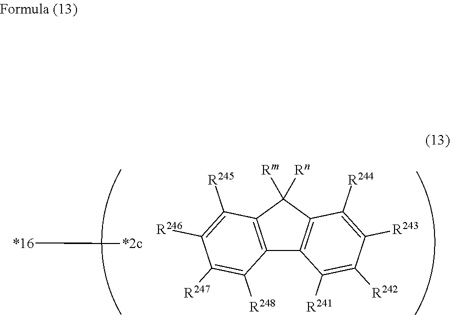

In the formula (13),
*16 is bonded to a central nitrogen atom;
R²⁴¹ to R²⁴⁸ are each independently a hydrogen atom, a substituted or unsubstituted alkyl group having 1 to 6 carbon atoms, a substituted or unsubstituted aryl group having 6 to 12 ring carbon atoms, or a substituted or unsubstituted aromatic heterocyclic group having 5 to 13 ring atoms;
provided that one selected from R²⁴¹ to R²⁴⁸ is a single bond bonded to *2c;
two adjacent ones selected from R²⁴¹ to R²⁴⁸ which are not single bonds may be bonded to each other to form one or plural substituted or unsubstituted benzene rings;
R^{m} and Rⁿ are each independently a substituted or unsubstituted alkyl group having 1 to 6 carbon atoms, or a substituted or unsubstituted aryl group having 6 to 12 ring carbon atoms; and
provided that R^{m} and Rⁿ may be bonded to each other to form a substituted or unsubstituted spiro ring.

The details of the substituted or unsubstituted alkyl group having 1 to 6 carbon atoms, the substituted or unsubstituted aryl group having 6 to 12 ring carbon atoms, and the substituted or unsubstituted aromatic heterocyclic group having 5 to 13 ring atoms are as described above.

It is preferable that both of R^{m} and Rⁿ are a methyl group or a phenyl group, or that R^{m} is a phenyl group and Rⁿ is a methyl group.

The details of the substituted or unsubstituted spiro ring formed by bonding R^{m} and Rⁿ to each other are as described for the spiro ring formed by bonding R^{A} and R^{B} to each other in formula (1).

Preferably one selected from R²⁴⁵ to R²⁴⁸, more preferably R²⁴⁶ is a single bond bonded to *2c.

All of R²⁴¹ to R²⁴⁸ which are not a single bond bonded to *2c may be a hydrogen atom.

The third compound of the present invention (inventive compound (3)) is represented by the following formula (3).

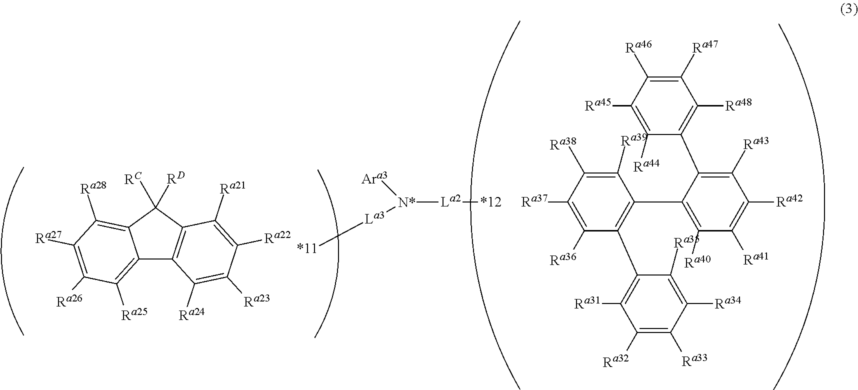

N* is a central nitrogen atom.
L^{a2}, L^{a3}. * 11, R^{a21} to R^{a24}, and R^{a25} to R^{a28} are each as defined in formula (1).

One selected from R^{a31} to R^{a48} is a single bond bonded to *12, and R^{a31} to R^{a48} which are not a single bond bonded to *12 are a hydrogen atom. Preferably R^{a31}, R^{a32}, R^{a33}, R^{a37}, or R^{a39}, more preferably R^{a31}, R^{a37}, or R^{a39} is a single bond bonded to *12.

Two adjacent ones selected from R^{a31} to R^{a48} which are not a single bond bonded to *12 may be bonded to each other to form a benzene ring, or may not be bonded to each other and therefore may not form a ring.

R^{C} and R^{D} are bonded to each other to form a substituted or unsubstituted spiro ring with the carbon atom at the 9-position of the fluorene skeleton. The details of the substituted or unsubstituted spiro ring are as described for the spiro ring formed by bonding R^{A} and R^{B} to each other in formula (1).

Ar^{a3} is a phenyl group or a naphthyl group which may be substituted with a triarylsilyl group having a substituted or unsubstituted aryl group having 6 to 30 ring carbon atoms, and an unsubstituted phenyl group or an unsubstituted naphthyl group are preferred.

The details of the substituted or unsubstituted aryl group having 6 to 30 ring carbon atoms in the triarylsilyl group are as described for R^{a21} to R^{a24} which are not a single bond bonded to *11, and R^{a25} to R^{a28}. The triarylsilyl group is preferably a triphenylsilyl group.

As mentioned above, “hydrogen atom” used in this description includes light hydrogen atoms, deuterium atoms, and tritium atoms. Accordingly, the inventive compounds may include naturally occurring deuterium atoms.

Further, a deuterium atom may be intentionally introduced into the inventive compound A by using a deuterated compound for a part of or all of the raw material compounds. Therefore, in one embodiment of the present invention, the inventive compounds contain at least one deuterium atom. That is, the inventive compound is a compound represented by any one of formulas (1) to (3), and may be a compound in which at least one of the hydrogen atoms contained in the compound is a deuterium atom.

At least one hydrogen atom selected from the following hydrogen atoms may be a deuterium atom. In addition, in the following, “substituted or unsubstituted”, the number of carbon atoms, and the number of atoms are omitted.
hydrogen atoms contained in the phenylene group, naphthylene group, or biphenylene group represented by L^{a1} in formula (1);
when L^{a2} or L^{a3} in the formula (1), (2) or (3) is a phenylene group, a naphthylene group, or a biphenylene group, hydrogen atoms contained in these groups;
hydrogen atoms represented by R^{a1} to R^{a5} which are not a single bond bonded to *10 in formula (1) or (2), and R^{a6} to R^{a18};
hydrogen atoms contained in the aryl group or aromatic heterocyclic group represented by Ar^{a1} in the formula (1);
hydrogen atoms represented by R^{a21} to R^{a24} which are not a single bond bonded to *11 in formula (1), (2) or (3), and R^{a25} to R^{a28};
when R^{a21} to R^{a24} which are not a single bond bonded to *11 in formula (1), (2) or (3), and R^{a25} to R^{a28} are alkyl groups, aryl groups, or aromatic heterocyclic groups, hydrogen atoms contained in these groups;
hydrogen atoms contained by a benzene ring arbitrarily formed by two adjacent ones selected from R^{a21} to R^{a24} which are not a single bond bonded to *11 in the formula (1), (2) or (3), and R^{a25} to R^{a28};
when R^{A} or R^{B} in the formula (1) or (2) is an alkyl group, an aryl group, or an aromatic heterocyclic group, hydrogen atoms contained in these groups;
when R^{A} or R^{B} of the formula (1) or (2) forms a spiro ring with the carbon atom at the 9-position of the fluorene skeleton, hydrogen atoms contained in the spiro ring;
hydrogen atoms contained in the condensed aryl group, the condensed aromatic heterocyclic group, or the fluorenyl group represented by Ar^{a2} in the formula (2);
hydrogen atoms represented by R^{a31} to R^{a48} which are not a single bond bonded to *12 in the formula (3);
hydrogen atoms contained in the spiro ring in which R^{C} and R^{D} of the formula (3) form a spiro ring with the carbon atom at the 9-position of the fluorene skeleton;
hydrogen atoms contained in the phenyl group or the naphthyl group represented by Ar^{a3} in the formula (3).

When Ar^{a3} in the formula (3) is a phenyl group or a naphthyl group substituted with a triarylsilyl group having a substituted or unsubstituted aryl group having 6 to 30 ring carbon atoms, hydrogen atoms contained in the aryl group.

The deuteration rate of the inventive compound depends on the deuteration rate of the raw material compounds used. Even when a raw material with a predetermined deuteration rate is used, a certain proportion of naturally occurring light hydrogen isotopes may be included. Therefore, the embodiment of the deuteration rate of the inventive compound represented below includes a ratio which takes into account trace amounts of naturally occurring isotopes, with respect to the ratio obtained by simply counting the number of deuterium atoms represented by the chemical formula.

The deuteration rate of the inventive compound is preferably 1% or more, more preferably 3% or more, further preferably 5% or more, further more preferably 10% or more, further more preferably 50% or more.

The inventive compound may be a mixture containing a deuterated compound and a non-deuterated compound, or a mixture of two or more compounds having different deuteration rate. The deuteration rate of these mixtures is preferably 1% or more, more preferably 3% or more, further preferably 5% or more, further more preferably 10% or more, further more preferably 50% or more, and less than 100%.

Further, the ratio of the number of deuterium atoms with respect to the total number of hydrogen atoms in the inventive compound is preferably 1% or more, more preferably 3% or more, further preferably 5% or more, further more preferably 10% or more, and 100% or less.

When the “substituted or unsubstituted XX group” included in the definitions of the above formulas is a substituted XX group, the details of the substituent are as described in “substituents in the case of “substituted or unsubstituted””, and is preferably an alkyl group having 1 to 6 carbon atoms, an aryl group having 6 to 12 ring atoms, or an aromatic heterocyclic group having 5 to 13 ring atoms, more preferably an alkyl group having 1 to 6 carbon atoms, or an aryl group having 6 to 12 ring carbon atoms. The details of each group are as described above.

The inventive compounds can be easily produced by those skilled in the art with reference to the following synthesis examples and known synthesis methods.

Specific examples of the inventive compounds are shown below, but the compounds are not limited to the following exemplified compounds.

In the following specific examples, D represents a deuterium atom.

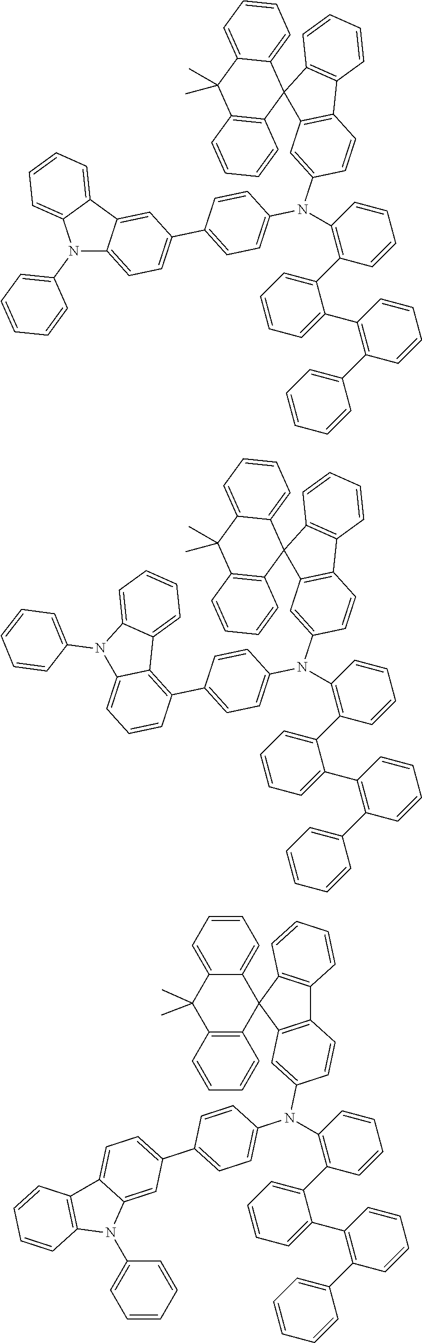

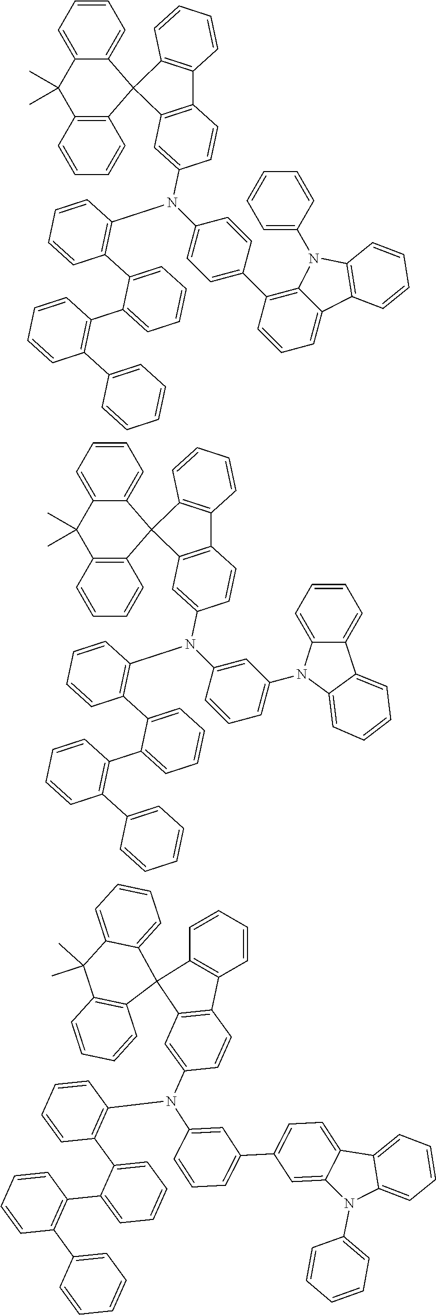

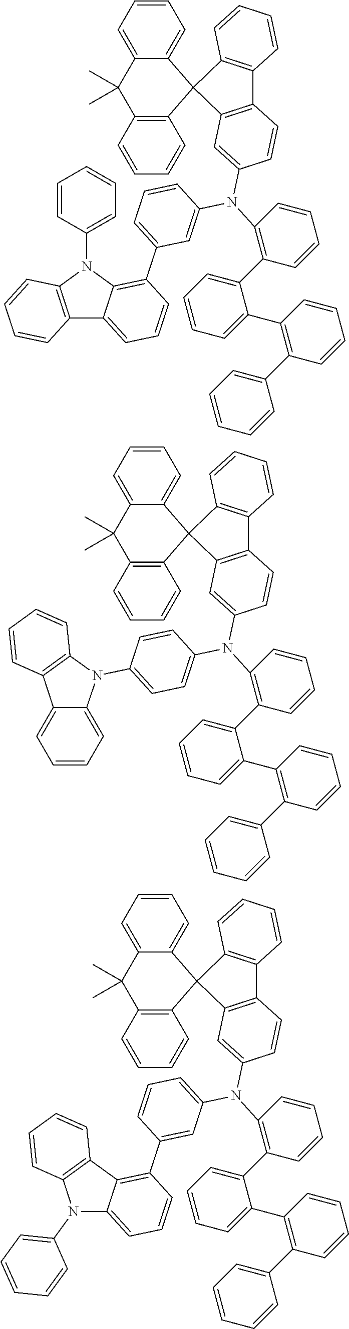

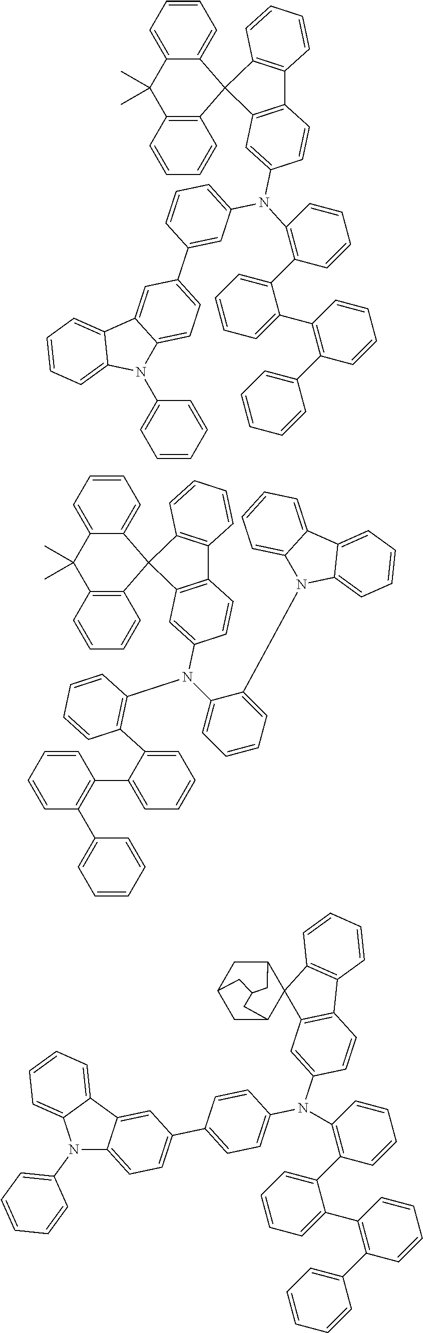

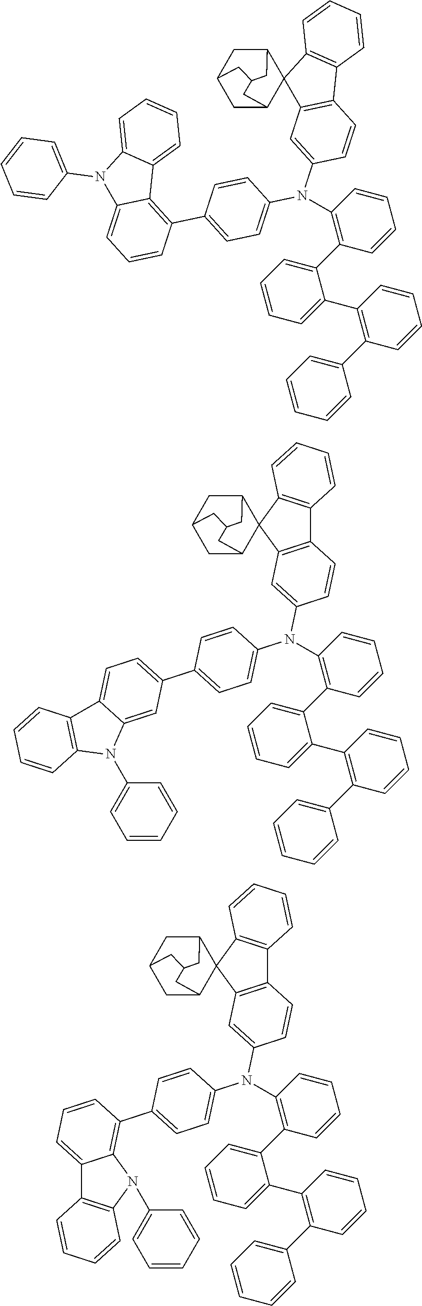

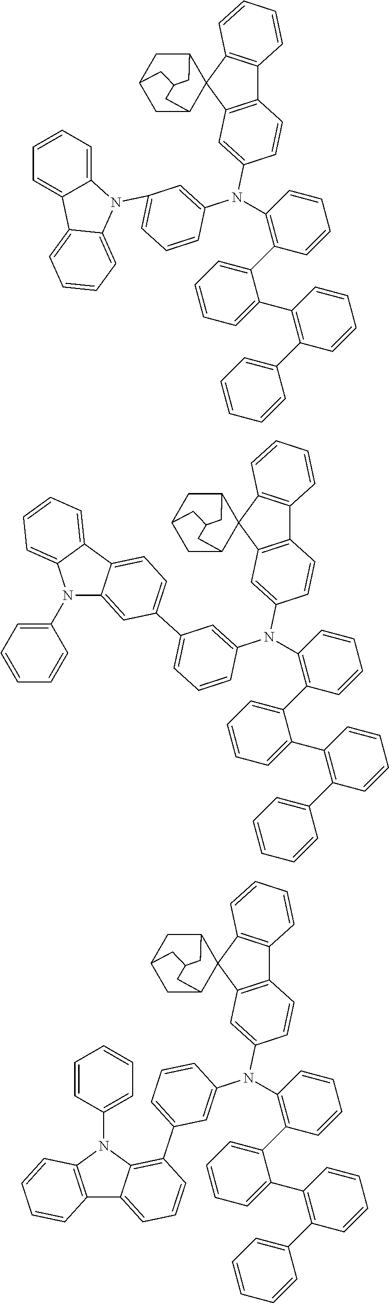

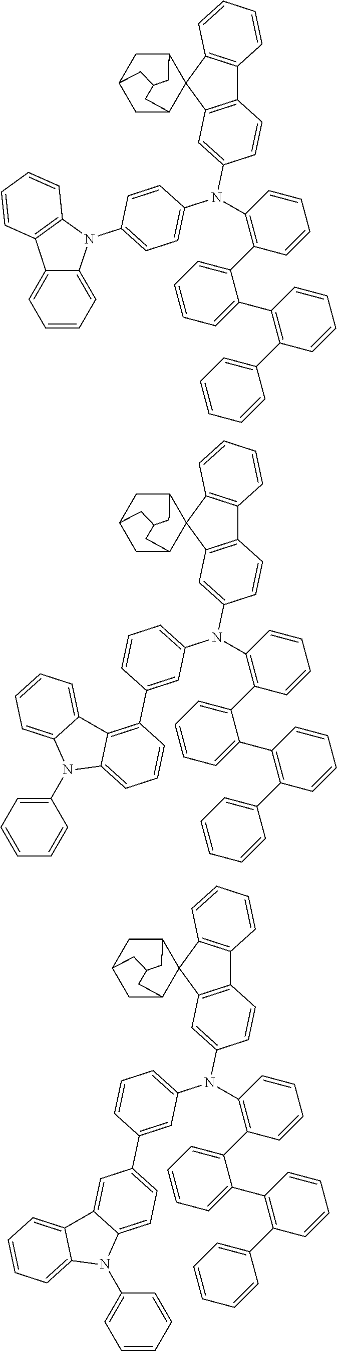

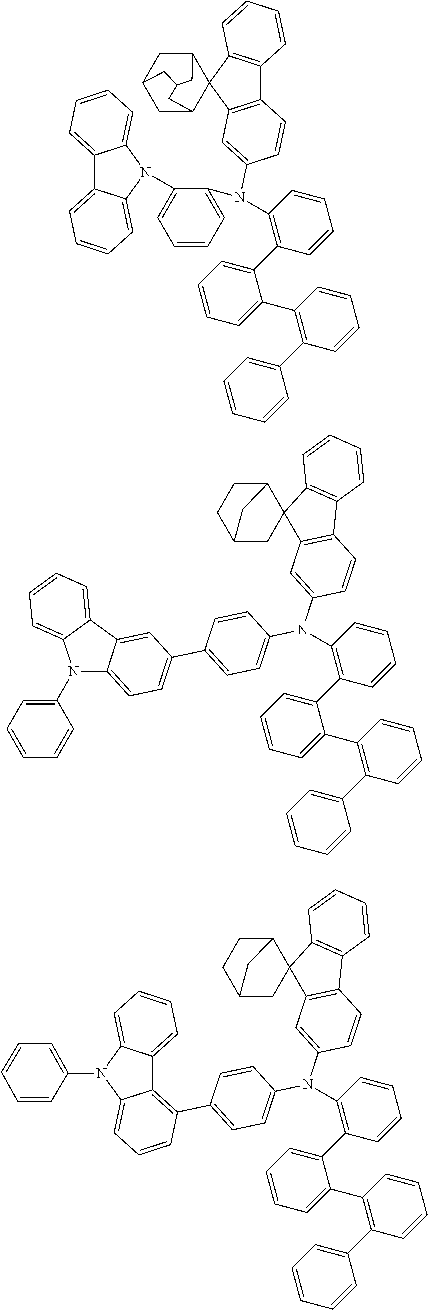

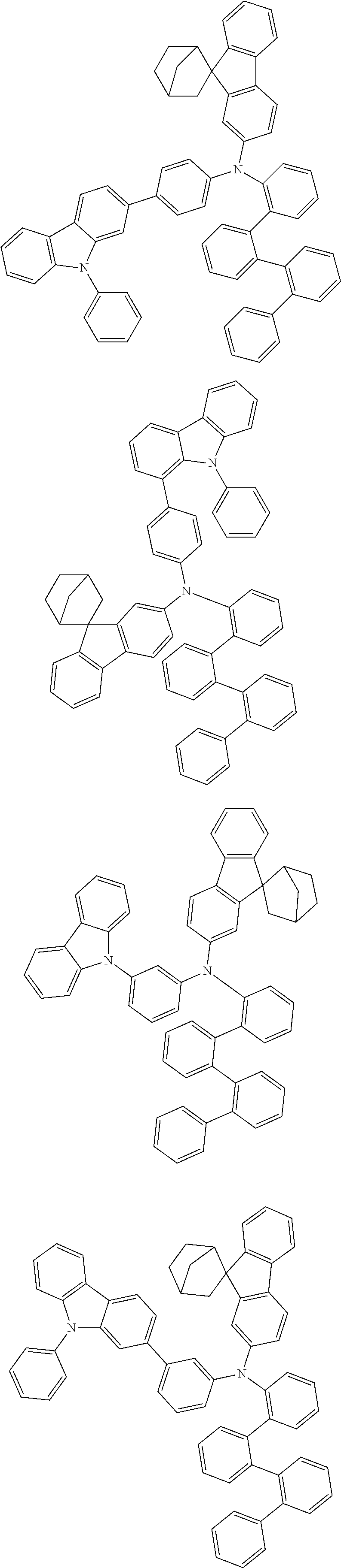

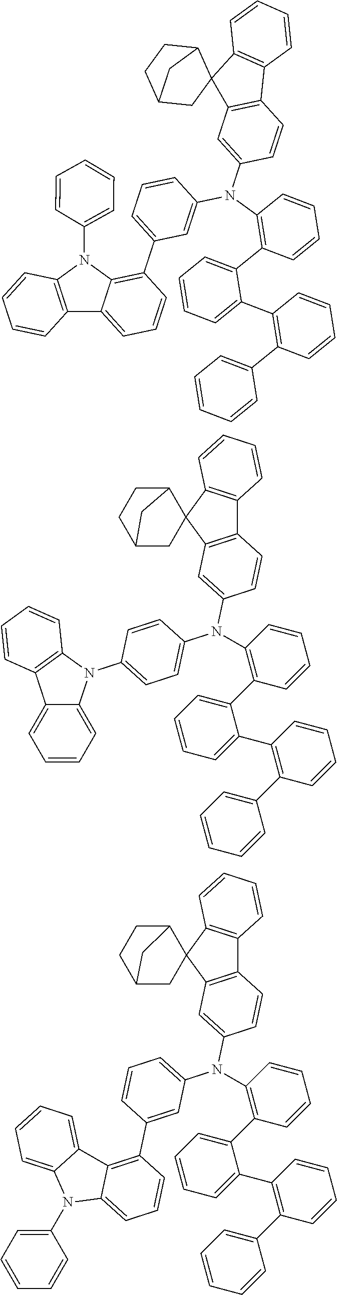

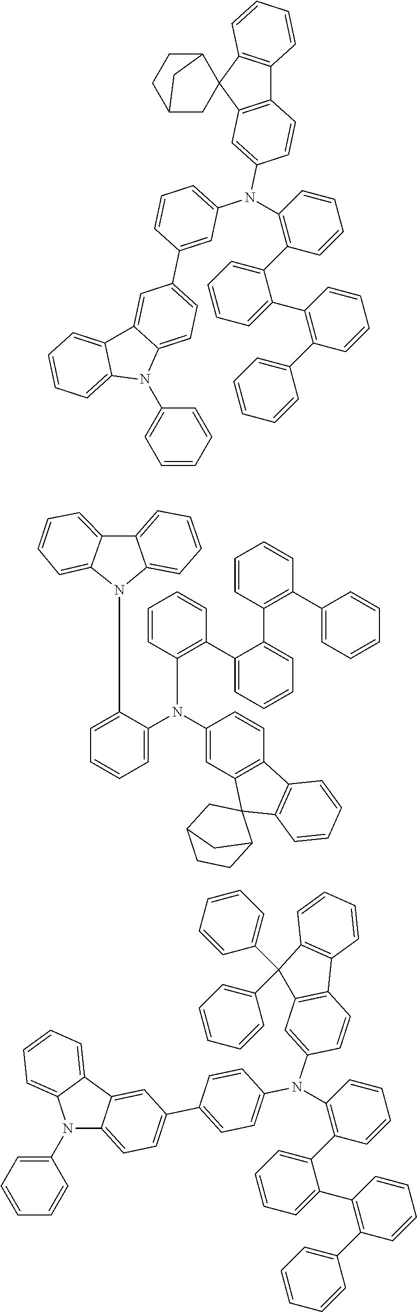

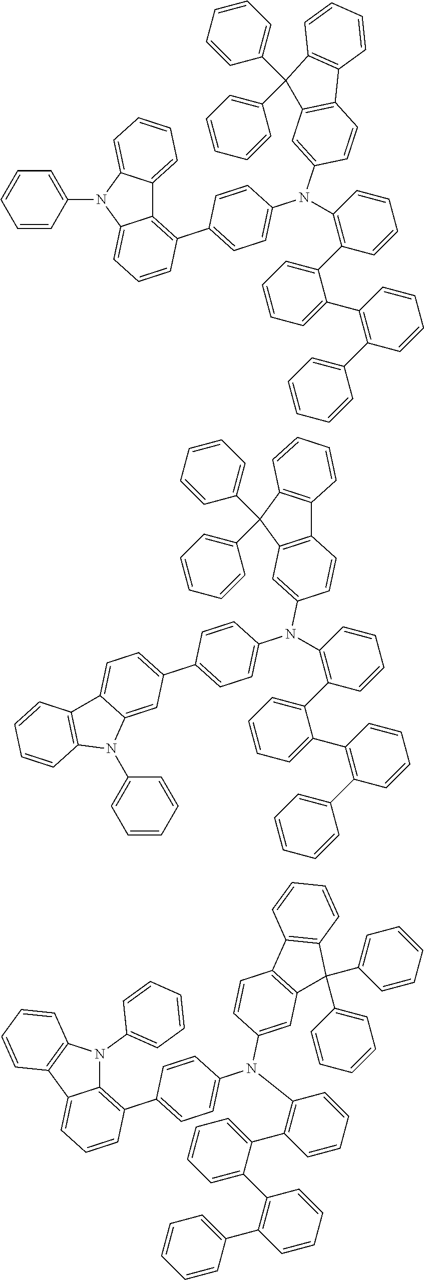

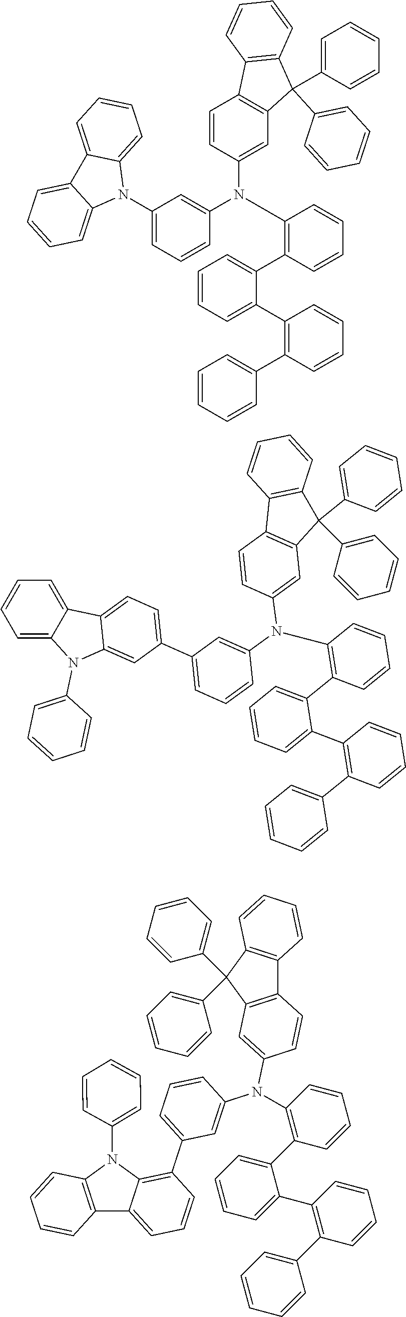

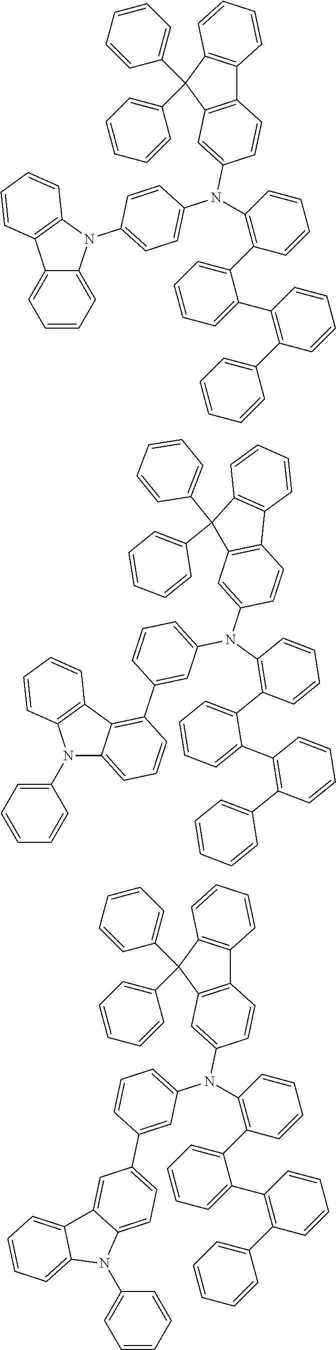

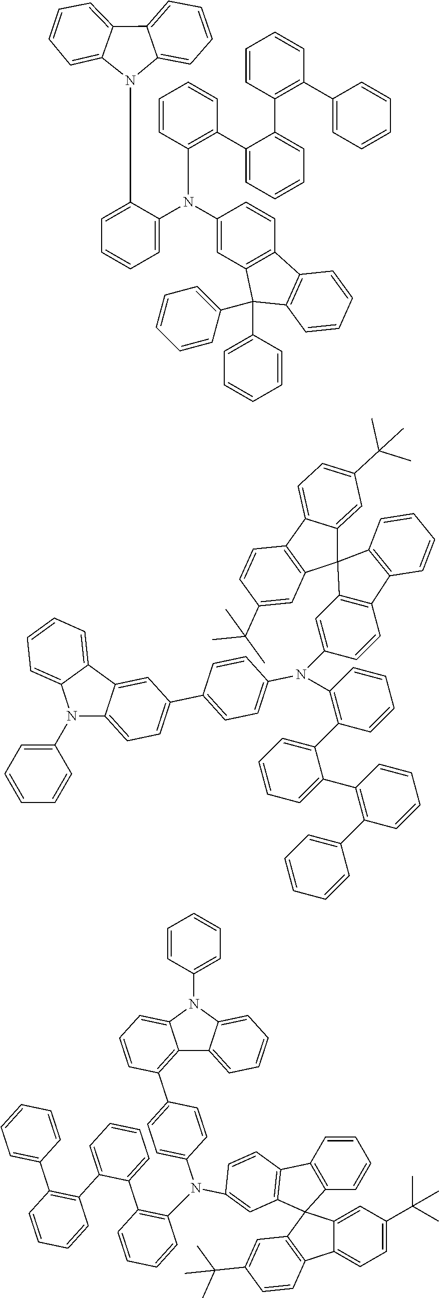 [SMILES: c1ccc(-c2ccccc2-c2ccccc2-c2ccccc2N(c2ccc3c(c2)C(c2ccccc2)(c2ccccc2)c2ccccc2-3)c2ccccc2-n2c3ccccc3c3ccccc32)cc1 ; CC(C)(C)c1ccc2c(c1)C1(c3ccccc3-c3ccc(N(c4ccc(-c5ccc6c(c5)c5ccccc5n6-c5ccccc5)cc4)c4ccccc4-c4ccccc4-c4ccccc4-c4ccccc4)cc31)c1cc(C(C)(C)C)ccc1-2 ; CC(C)(C)c1ccc2c(c1)C1(c3ccccc3-c3ccc(N(c4ccc(-c5cccc6c5c5ccccc5n6-c5ccccc5)cc4)c4ccccc4-c4ccccc4-c4ccccc4-c4ccccc4)cc31)c1cc(C(C)(C)C)ccc1-2]

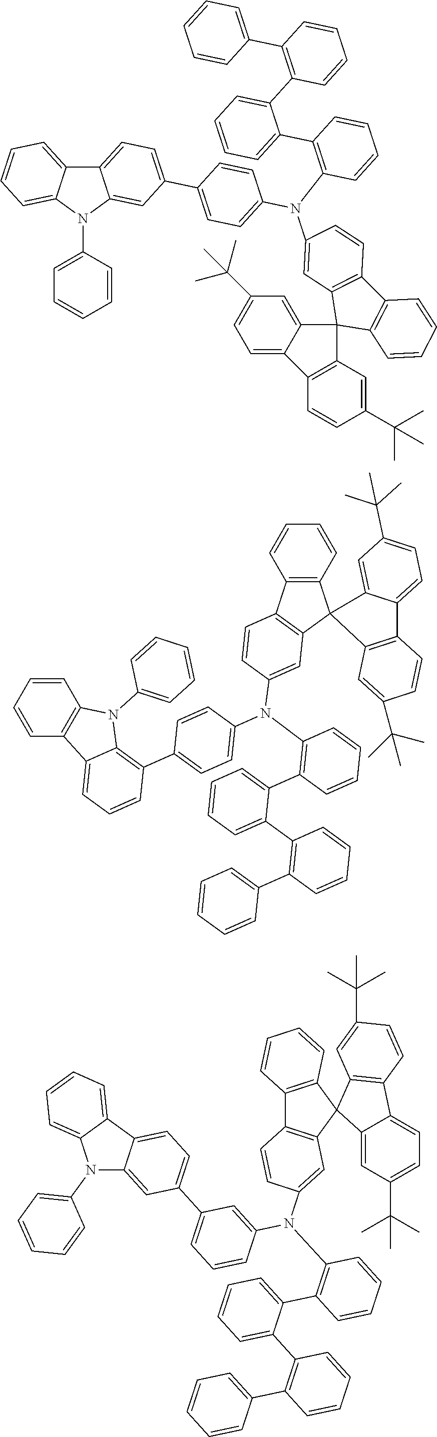

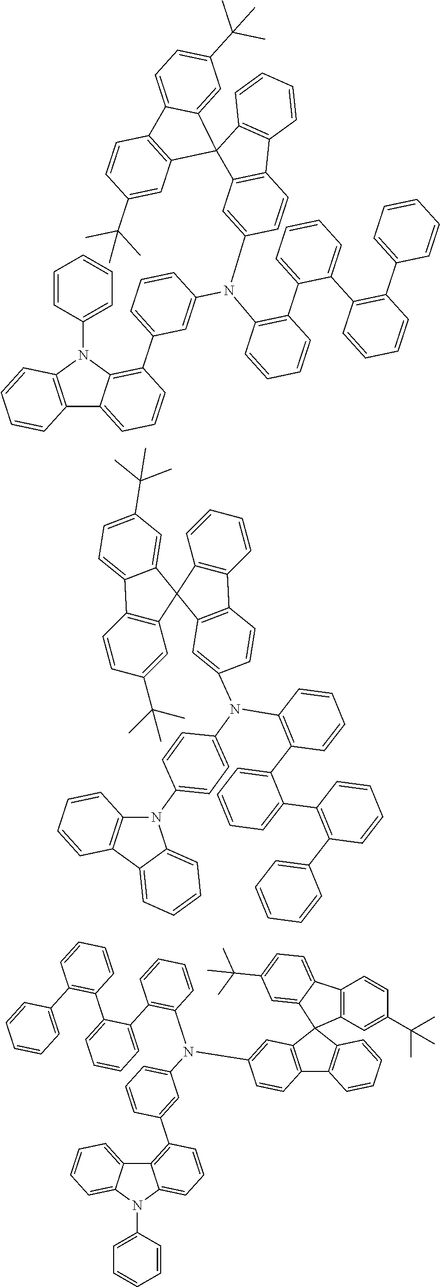

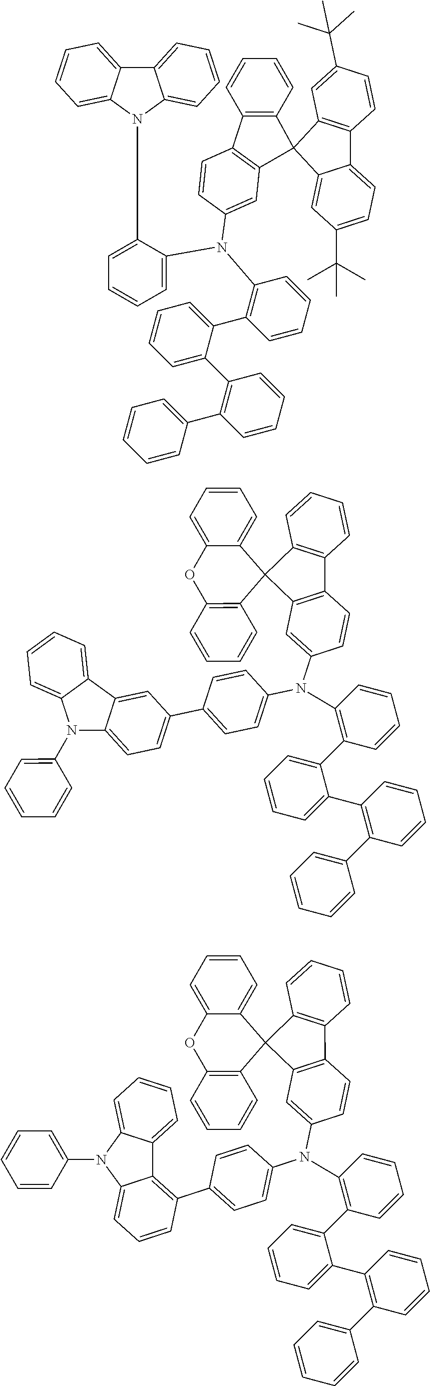

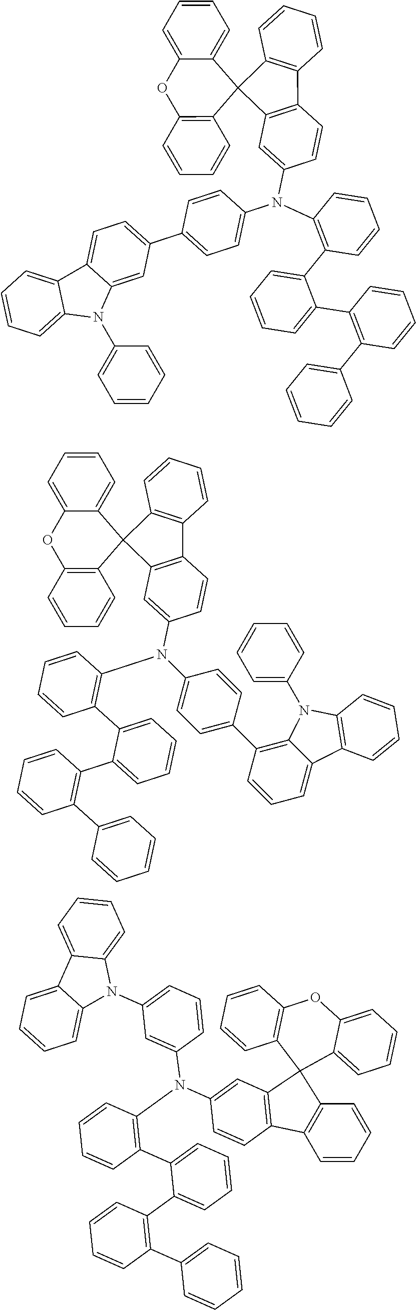

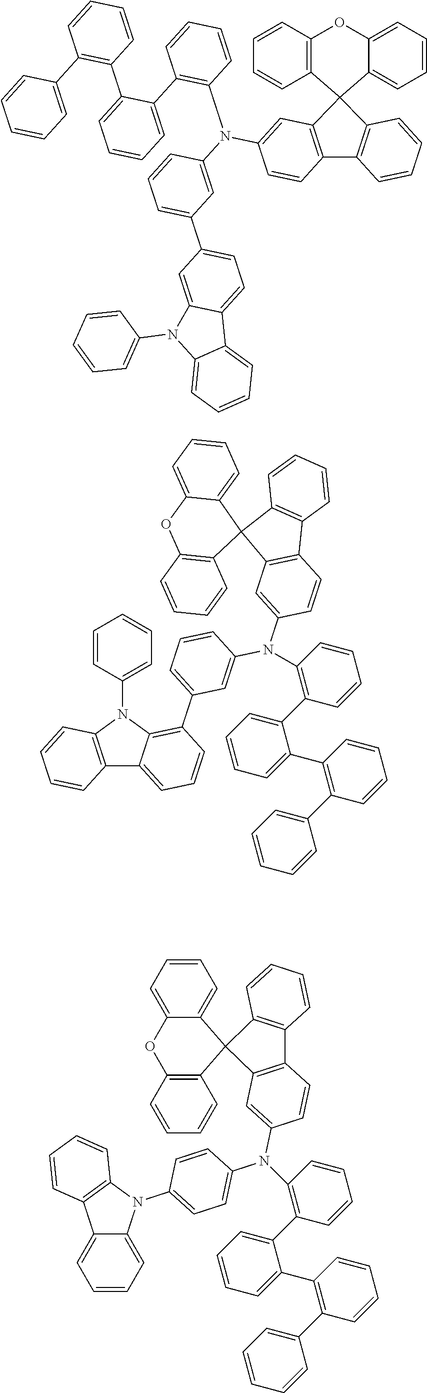

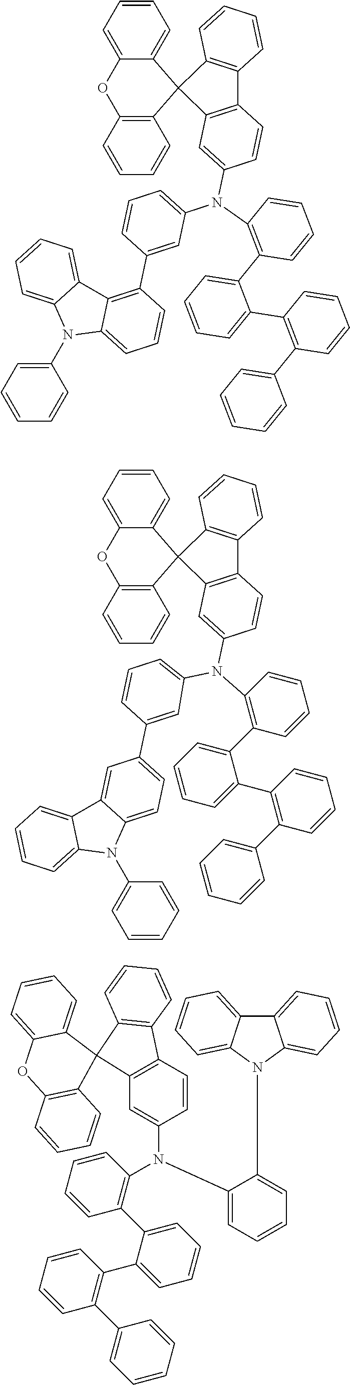

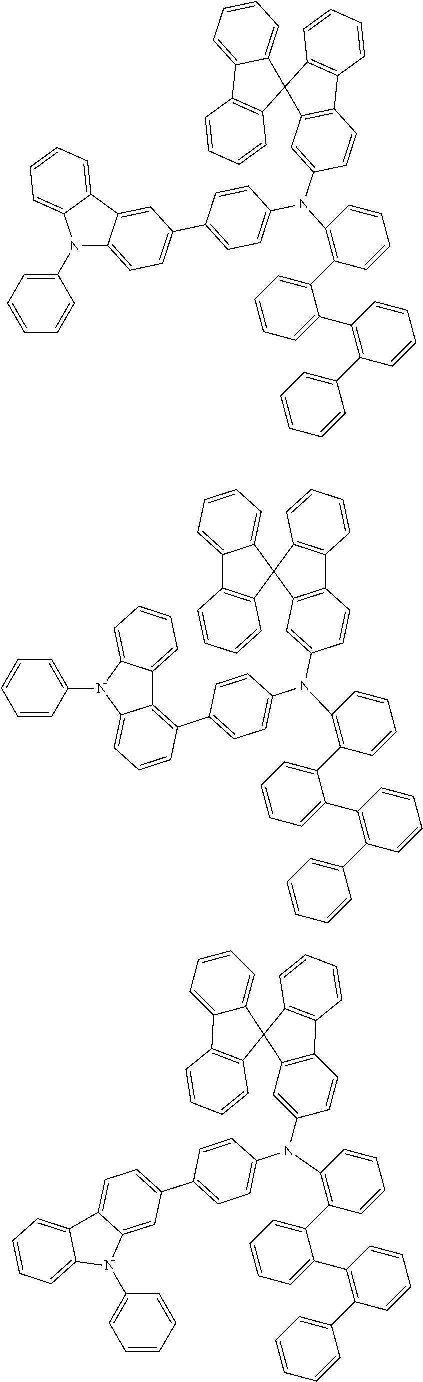

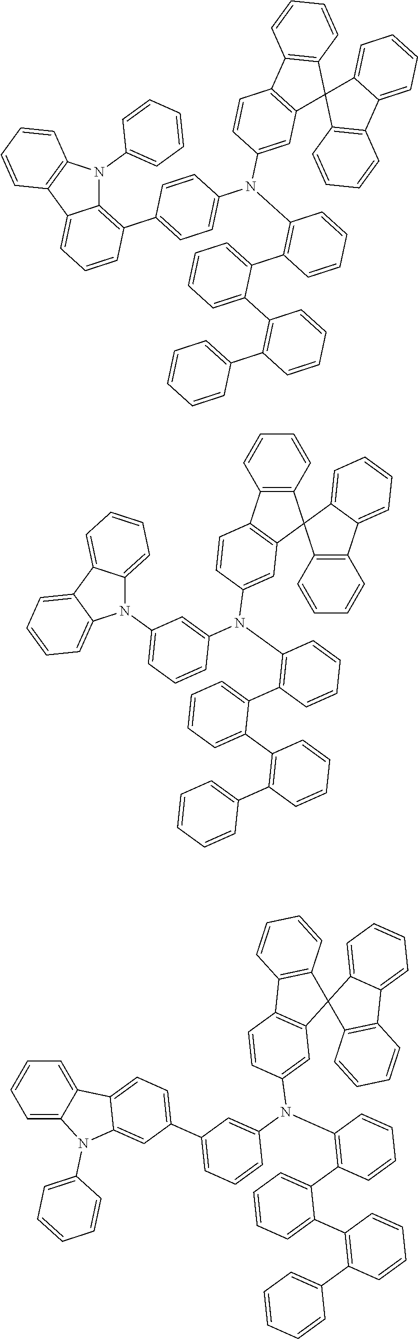 [SMILES: c1ccc(-c2ccccc2-c2ccccc2-c2ccccc2N(c2ccc(-c3cccc4c5ccccc5n(-c5ccccc5)c34)cc2)c2ccc3c(c2)C2(c4ccccc4-c4ccccc42)c2ccccc2-3)cc1 ; c1ccc(-c2ccccc2-c2ccccc2-c2ccccc2N(c2cccc(-n3c4ccccc4c4ccccc43)c2)c2ccc3c(c2)C2(c4ccccc4-c4ccccc42)c2ccccc2-3)cc1 ; c1ccc(-c2ccccc2-c2ccccc2-c2ccccc2N(c2cccc(-c3ccc4c5ccccc5n(-c5ccccc5)c4c3)c2)c2ccc3c(c2)C2(c4ccccc4-c4ccccc42)c2ccccc2-3)cc1]

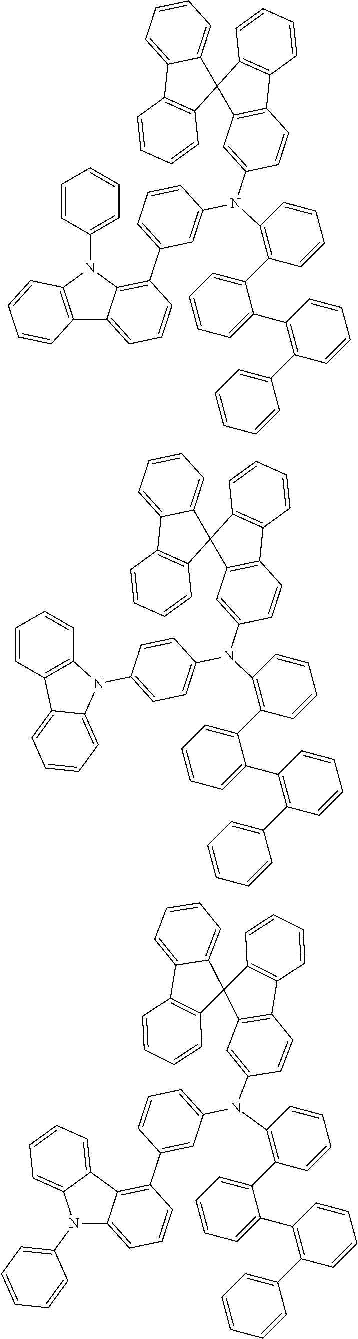

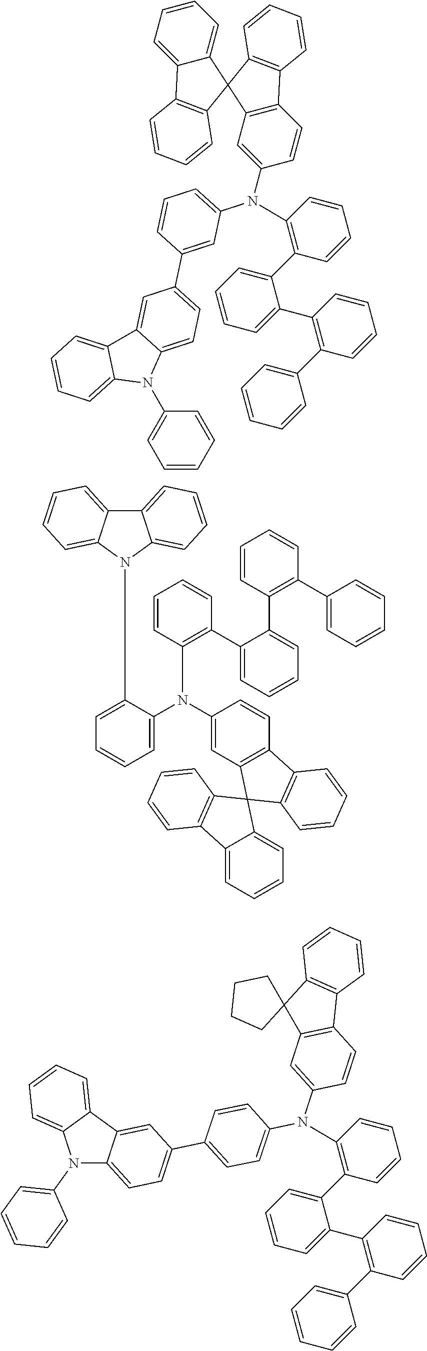

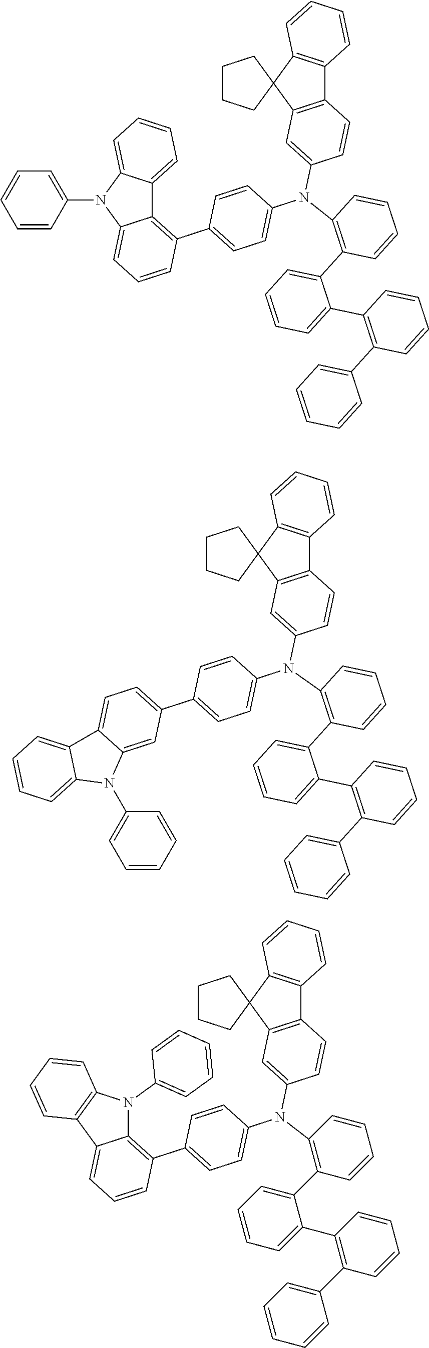

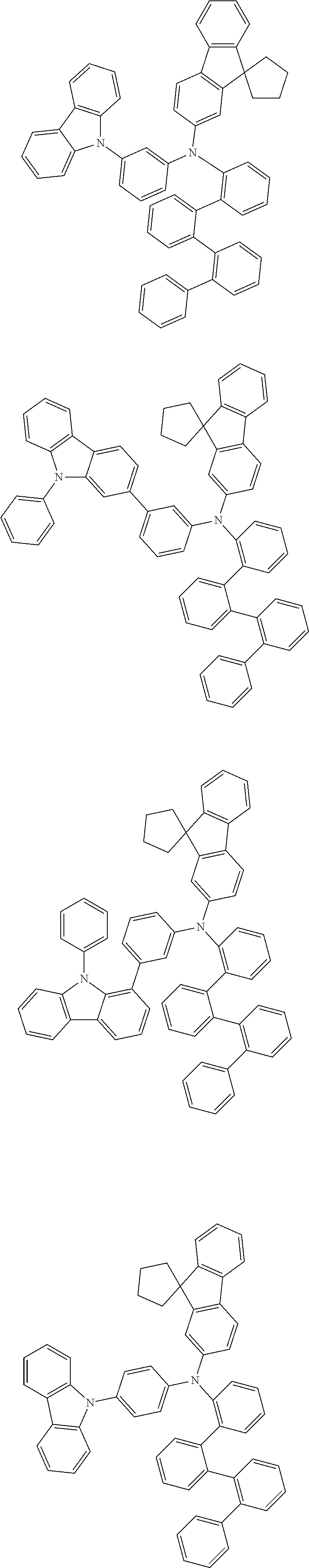 [SMILES: c1ccc(-c2ccccc2-c2ccccc2-c2ccccc2N(c2cccc(-n3c4ccccc4c4ccccc43)c2)c2ccc3c(c2)C2(CCCC2)c2ccccc2-3)cc1 ; c1ccc(-c2ccccc2-c2ccccc2-c2ccccc2N(c2cccc(-c3ccc4c5ccccc5n(-c5ccccc5)c4c3)c2)c2ccc3c(c2)C2(CCCC2)c2ccccc2-3)cc1 ; c1ccc(-c2ccccc2-c2ccccc2-c2ccccc2N(c2cccc(-c3cccc4c5ccccc5n(-c5ccccc5)c34)c2)c2ccc3c(c2)C2(CCCC2)c2ccccc2-3)cc1 ; c1ccc(-c2ccccc2-c2ccccc2-c2ccccc2N(c2ccc(-n3c4ccccc4c4ccccc43)cc2)c2ccc3c(c2)C2(CCCC2)c2ccccc2-3)cc1]

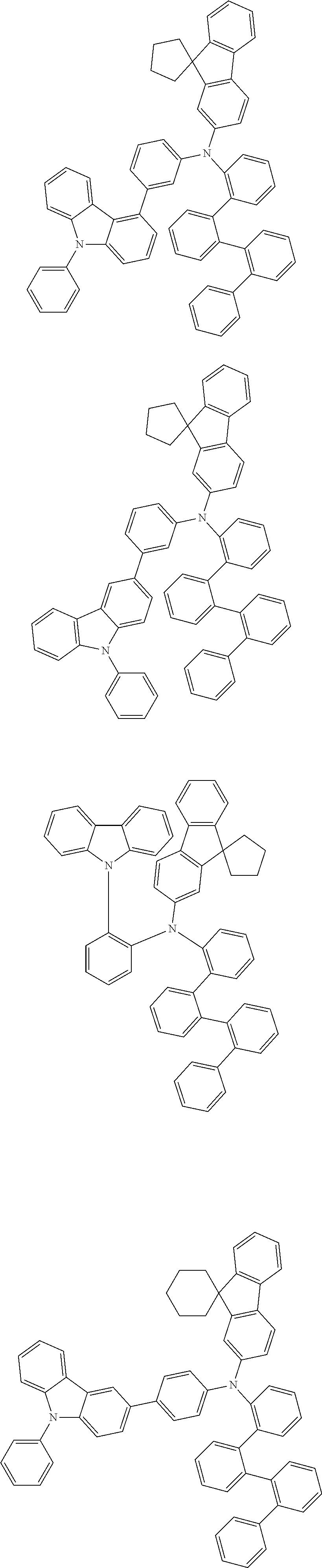

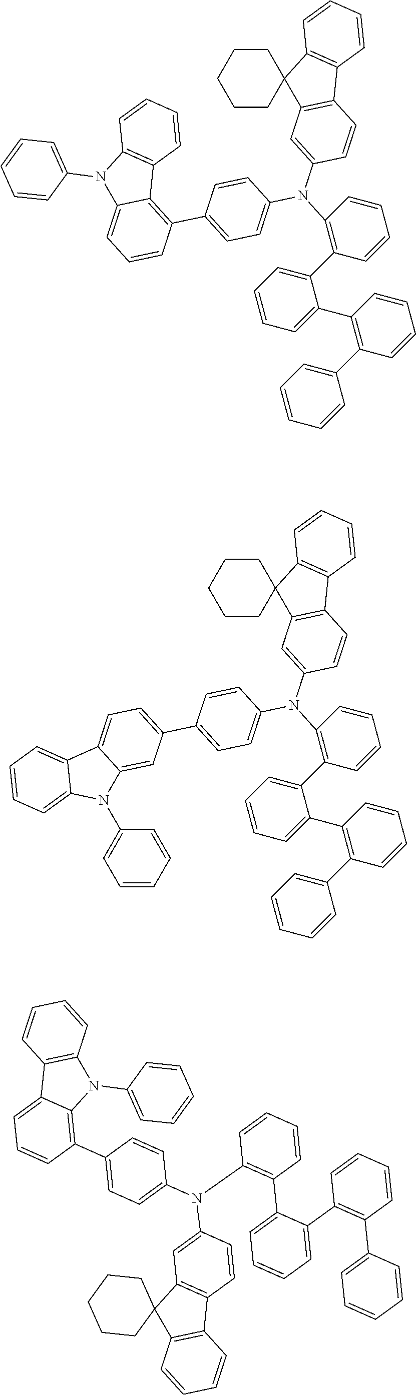 [SMILES: c1ccc(-c2ccccc2-c2ccccc2-c2ccccc2N(c2ccc(-c3cccc4c3c3ccccc3n4-c3ccccc3)cc2)c2ccc3c(c2)C2(CCCCC2)c2ccccc2-3)cc1 ; c1ccc(-c2ccccc2-c2ccccc2-c2ccccc2N(c2ccc(-c3ccc4c5ccccc5n(-c5ccccc5)c4c3)cc2)c2ccc3c(c2)C2(CCCCC2)c2ccccc2-3)cc1 ; c1ccc(-c2ccccc2-c2ccccc2-c2ccccc2N(c2ccc(-c3cccc4c5ccccc5n(-c5ccccc5)c34)cc2)c2ccc3c(c2)C2(CCCCC2)c2ccccc2-3)cc1]

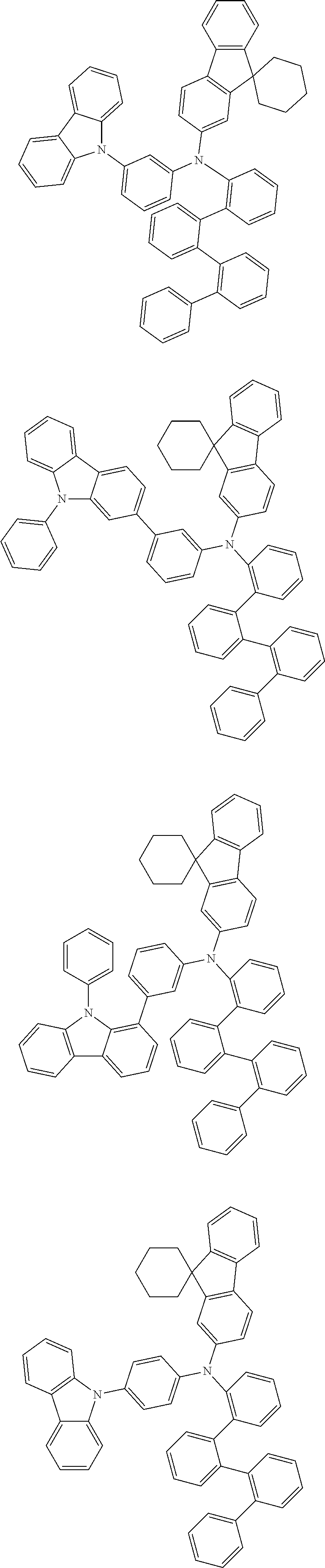 [SMILES: c1ccc(-c2ccccc2-c2ccccc2-c2ccccc2N(c2cccc(-n3c4ccccc4c4ccccc43)c2)c2ccc3c(c2)C2(CCCCC2)c2ccccc2-3)cc1 ; c1ccc(-c2ccccc2-c2ccccc2-c2ccccc2N(c2cccc(-c3ccc4c5ccccc5n(-c5ccccc5)c4c3)c2)c2ccc3c(c2)C2(CCCCC2)c2ccccc2-3)cc1 ; c1ccc(-c2ccccc2-c2ccccc2-c2ccccc2N(c2cccc(-c3cccc4c5ccccc5n(-c5ccccc5)c34)c2)c2ccc3c(c2)C2(CCCCC2)c2ccccc2-3)cc1 ; c1ccc(-c2ccccc2-c2ccccc2-c2ccccc2N(c2ccc(-n3c4ccccc4c4ccccc43)cc2)c2ccc3c(c2)C2(CCCCC2)c2ccccc2-3)cc1]

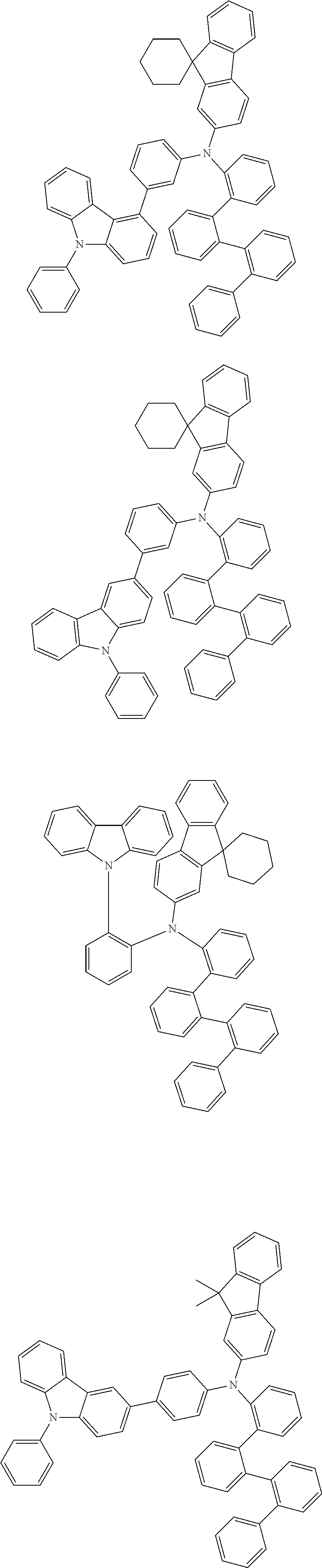

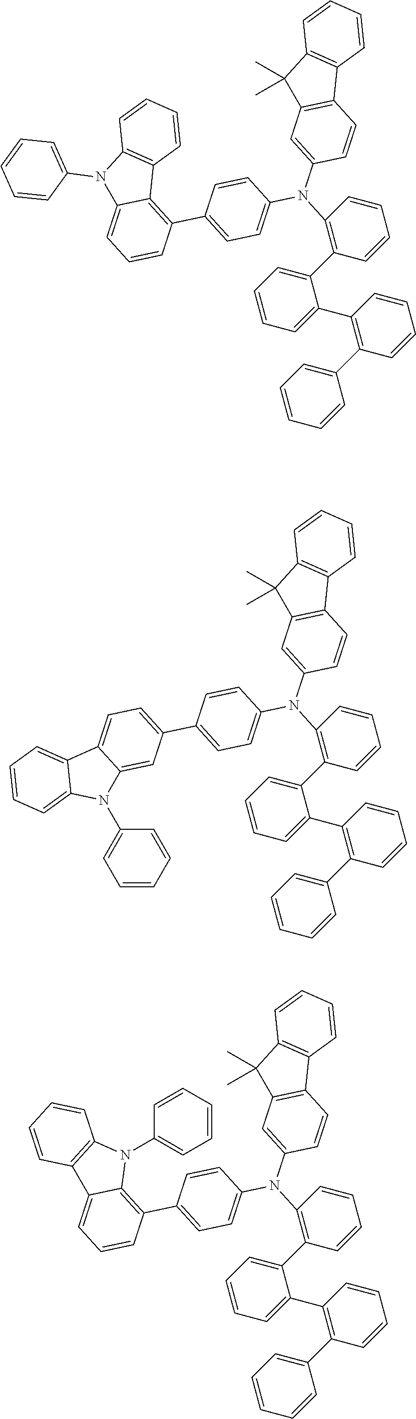 [SMILES: CC1(C)c2ccccc2-c2ccc(N(c3ccc(-c4cccc5c4c4ccccc4n5-c4ccccc4)cc3)c3ccccc3-c3ccccc3-c3ccccc3-c3ccccc3)cc21 ; CC1(C)c2ccccc2-c2ccc(N(c3ccc(-c4ccc5c6ccccc6n(-c6ccccc6)c5c4)cc3)c3ccccc3-c3ccccc3-c3ccccc3-c3ccccc3)cc21 ; CC1(C)c2ccccc2-c2ccc(N(c3ccc(-c4cccc5c6ccccc6n(-c6ccccc6)c45)cc3)c3ccccc3-c3ccccc3-c3ccccc3-c3ccccc3)cc21]

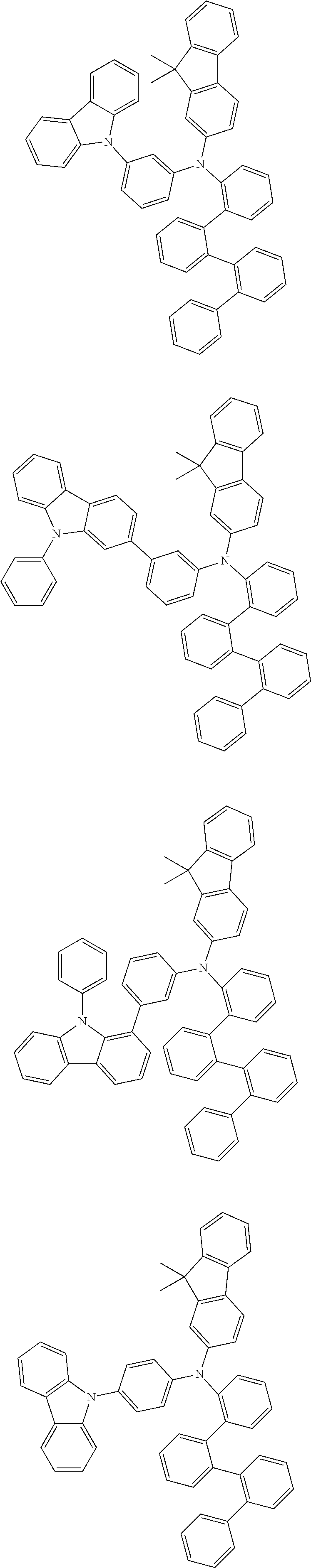 [SMILES: CC1(C)c2ccccc2-c2ccc(N(c3cccc(-n4c5ccccc5c5ccccc54)c3)c3ccccc3-c3ccccc3-c3ccccc3-c3ccccc3)cc21 ; CC1(C)c2ccccc2-c2ccc(N(c3cccc(-c4ccc5c6ccccc6n(-c6ccccc6)c5c4)c3)c3ccccc3-c3ccccc3-c3ccccc3-c3ccccc3)cc21 ; CC1(C)c2ccccc2-c2ccc(N(c3cccc(-c4cccc5c6ccccc6n(-c6ccccc6)c45)c3)c3ccccc3-c3ccccc3-c3ccccc3-c3ccccc3)cc21 ; CC1(C)c2ccccc2-c2ccc(N(c3ccc(-n4c5ccccc5c5ccccc54)cc3)c3ccccc3-c3ccccc3-c3ccccc3-c3ccccc3)cc21]

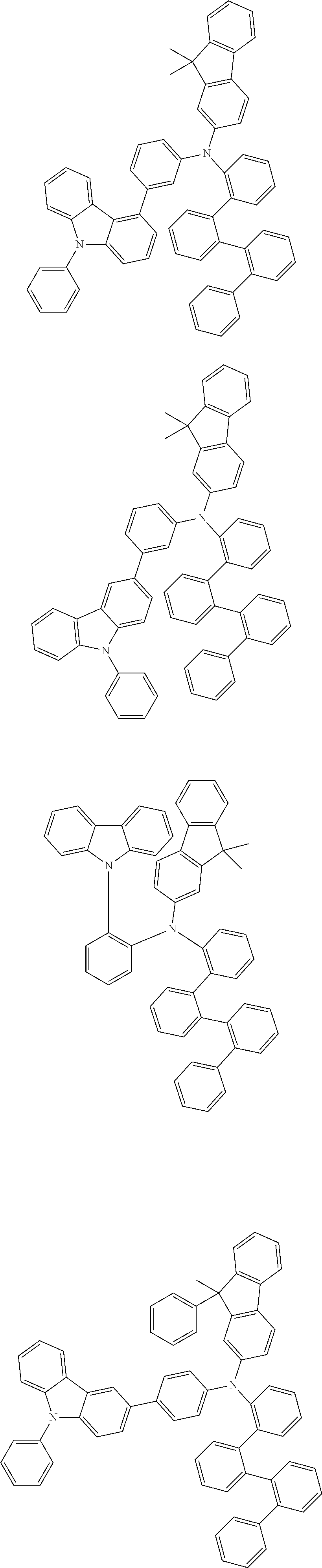

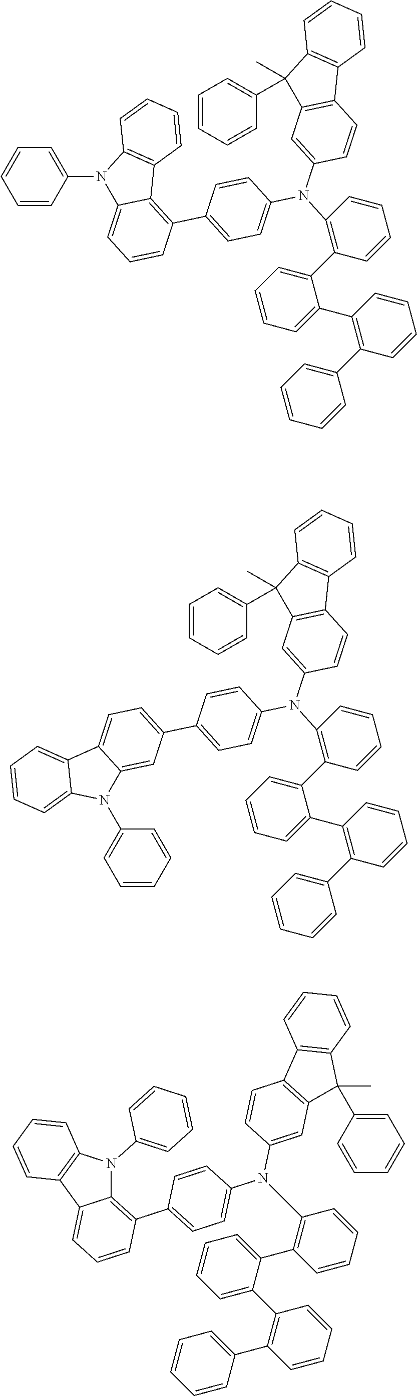 [SMILES: CC1(c2ccccc2)c2ccccc2-c2ccc(N(c3ccc(-c4cccc5c4c4ccccc4n5-c4ccccc4)cc3)c3ccccc3-c3ccccc3-c3ccccc3-c3ccccc3)cc21 ; CC1(c2ccccc2)c2ccccc2-c2ccc(N(c3ccc(-c4ccc5c6ccccc6n(-c6ccccc6)c5c4)cc3)c3ccccc3-c3ccccc3-c3ccccc3-c3ccccc3)cc21 ; CC1(c2ccccc2)c2ccccc2-c2ccc(N(c3ccc(-c4cccc5c6ccccc6n(-c6ccccc6)c45)cc3)c3ccccc3-c3ccccc3-c3ccccc3-c3ccccc3)cc21]

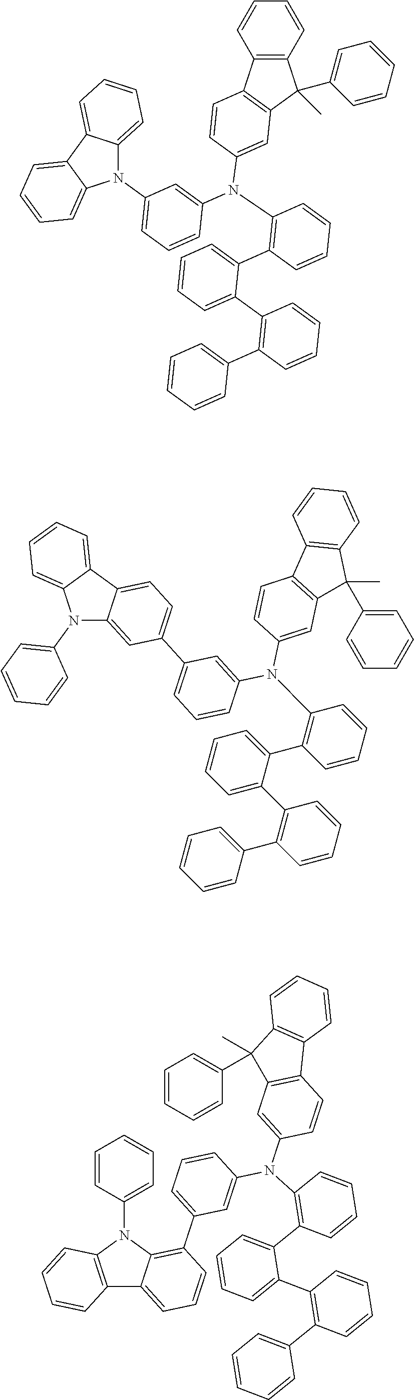 [SMILES: CC1(c2ccccc2)c2ccccc2-c2ccc(N(c3cccc(-n4c5ccccc5c5ccccc54)c3)c3ccccc3-c3ccccc3-c3ccccc3-c3ccccc3)cc21 ; CC1(c2ccccc2)c2ccccc2-c2ccc(N(c3cccc(-c4ccc5c6ccccc6n(-c6ccccc6)c5c4)c3)c3ccccc3-c3ccccc3-c3ccccc3-c3ccccc3)cc21 ; CC1(c2ccccc2)c2ccccc2-c2ccc(N(c3cccc(-c4cccc5c6ccccc6n(-c6ccccc6)c45)c3)c3ccccc3-c3ccccc3-c3ccccc3-c3ccccc3)cc21]

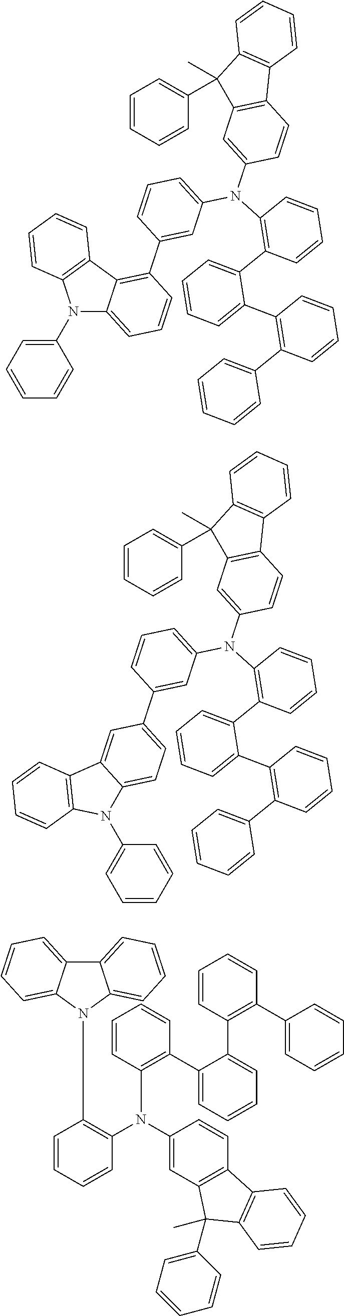

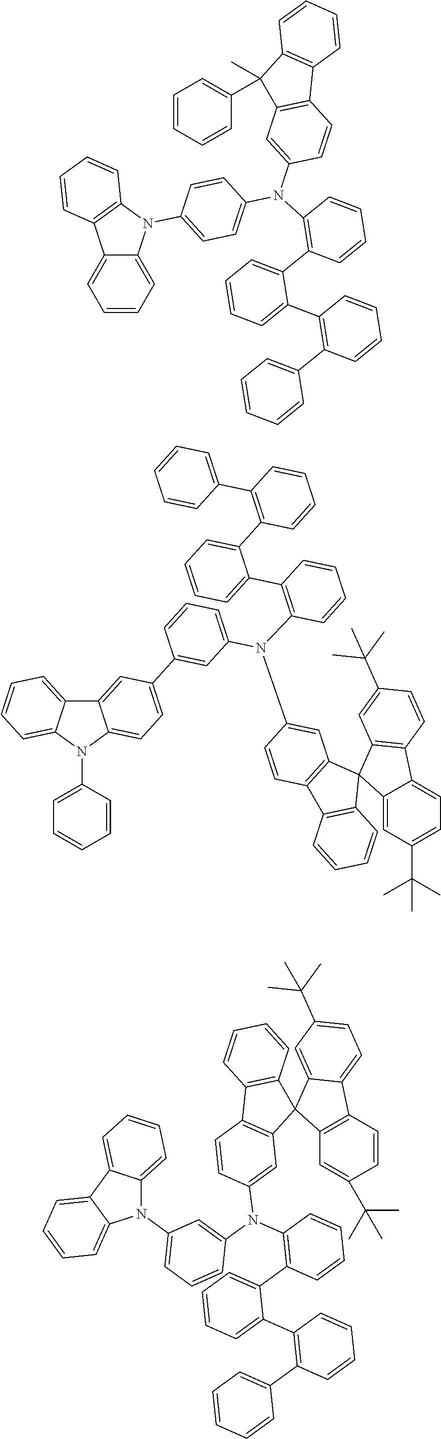

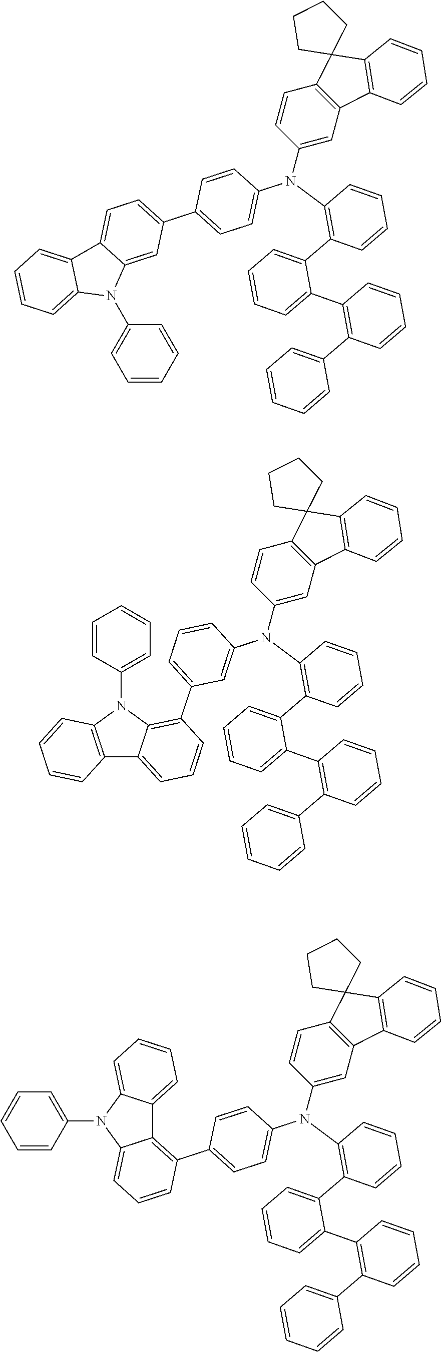

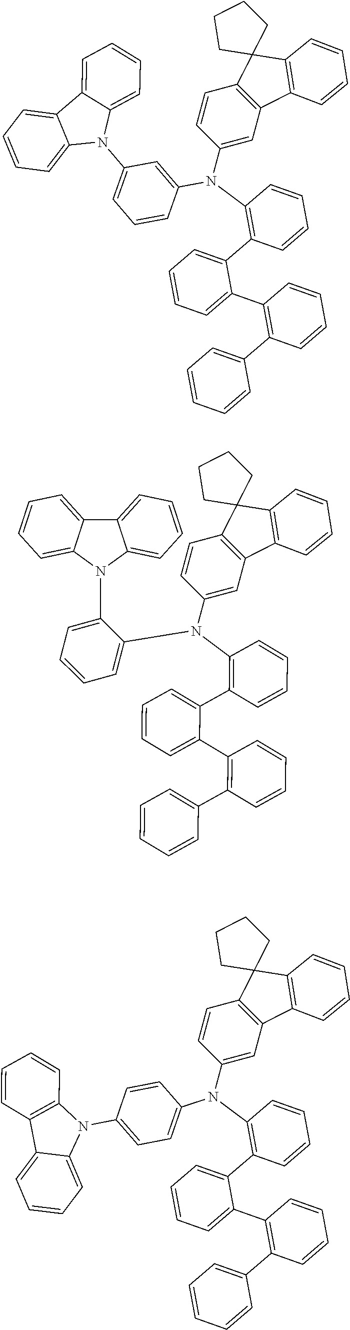

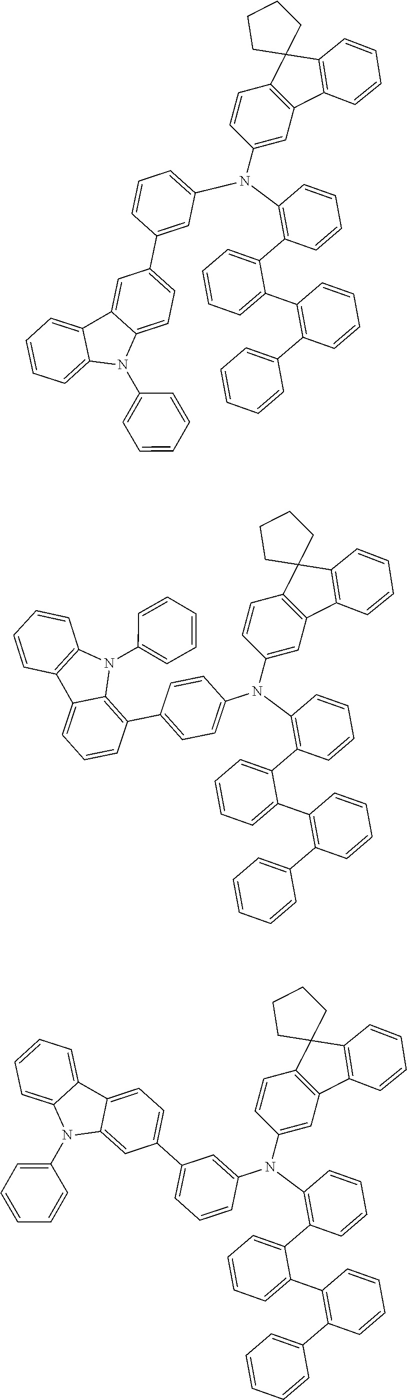 [SMILES: c1ccc(-c2ccccc2-c2ccccc2-c2ccccc2N(c2cccc(-c3ccc4c(c3)c3ccccc3n4-c3ccccc3)c2)c2ccc3c(c2)-c2ccccc2C32CCCC2)cc1 ; c1ccc(-c2ccccc2-c2ccccc2-c2ccccc2N(c2ccc(-c3cccc4c5ccccc5n(-c5ccccc5)c34)cc2)c2ccc3c(c2)-c2ccccc2C32CCCC2)cc1 ; c1ccc(-c2ccccc2-c2ccccc2-c2ccccc2N(c2cccc(-c3ccc4c5ccccc5n(-c5ccccc5)c4c3)c2)c2ccc3c(c2)-c2ccccc2C32CCCC2)cc1]

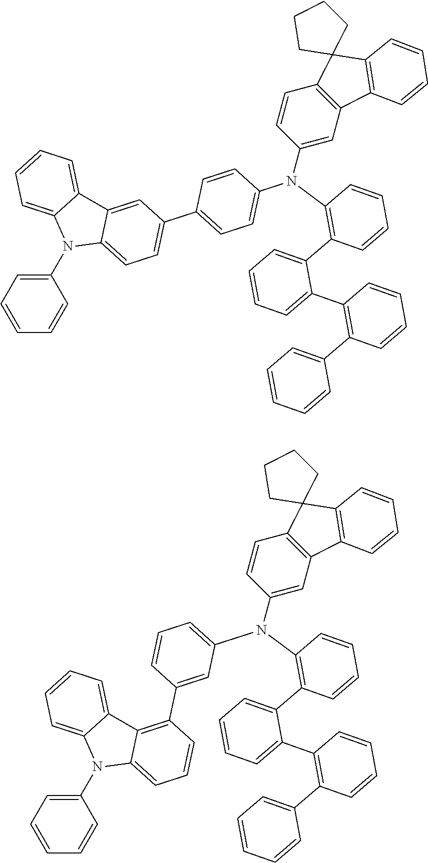

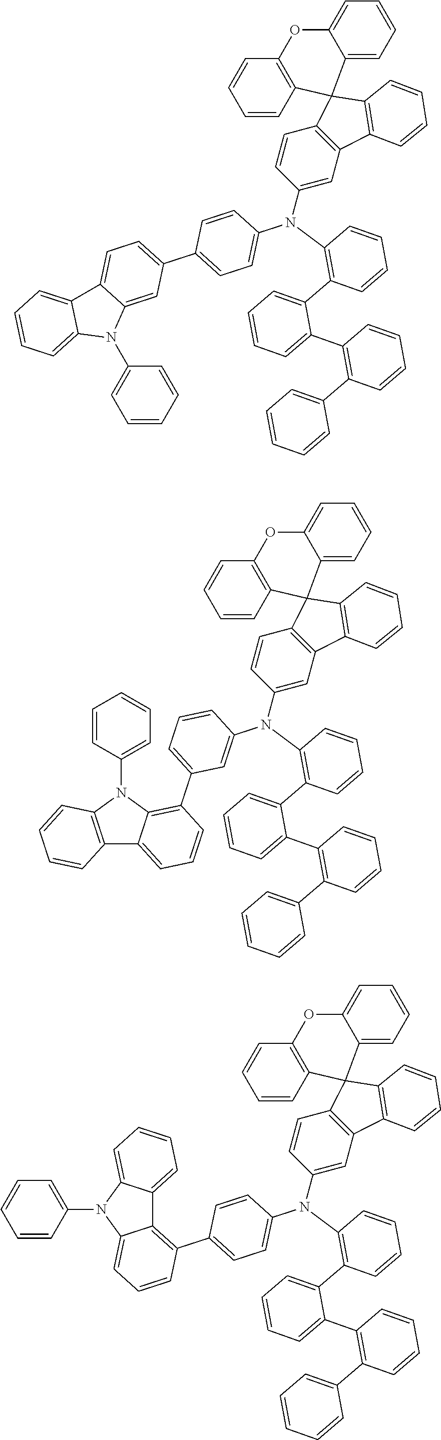 [SMILES: c1ccc(-c2ccccc2-c2ccccc2-c2ccccc2N(c2ccc(-c3ccc4c5ccccc5n(-c5ccccc5)c4c3)cc2)c2ccc3c(c2)-c2ccccc2C32c3ccccc3Oc3ccccc32)cc1 ; c1ccc(-c2ccccc2-c2ccccc2-c2ccccc2N(c2cccc(-c3cccc4c5ccccc5n(-c5ccccc5)c34)c2)c2ccc3c(c2)-c2ccccc2C32c3ccccc3Oc3ccccc32)cc1 ; c1ccc(-c2ccccc2-c2ccccc2-c2ccccc2N(c2ccc(-c3cccc4c3c3ccccc3n4-c3ccccc3)cc2)c2ccc3c(c2)-c2ccccc2C32c3ccccc3Oc3ccccc32)cc1]

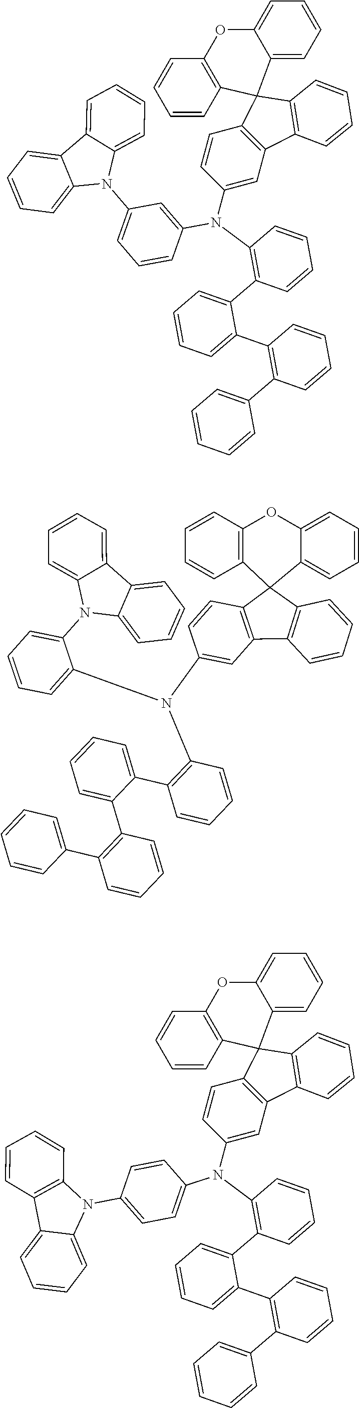 [SMILES: c1ccc(-c2ccccc2-c2ccccc2-c2ccccc2N(c2cccc(-n3c4ccccc4c4ccccc43)c2)c2ccc3c(c2)-c2ccccc2C32c3ccccc3Oc3ccccc32)cc1 ; c1ccc(-c2ccccc2-c2ccccc2-c2ccccc2N(c2ccc3c(c2)-c2ccccc2C32c3ccccc3Oc3ccccc32)c2ccccc2-n2c3ccccc3c3ccccc32)cc1 ; c1ccc(-c2ccccc2-c2ccccc2-c2ccccc2N(c2ccc(-n3c4ccccc4c4ccccc43)cc2)c2ccc3c(c2)-c2ccccc2C32c3ccccc3Oc3ccccc32)cc1]

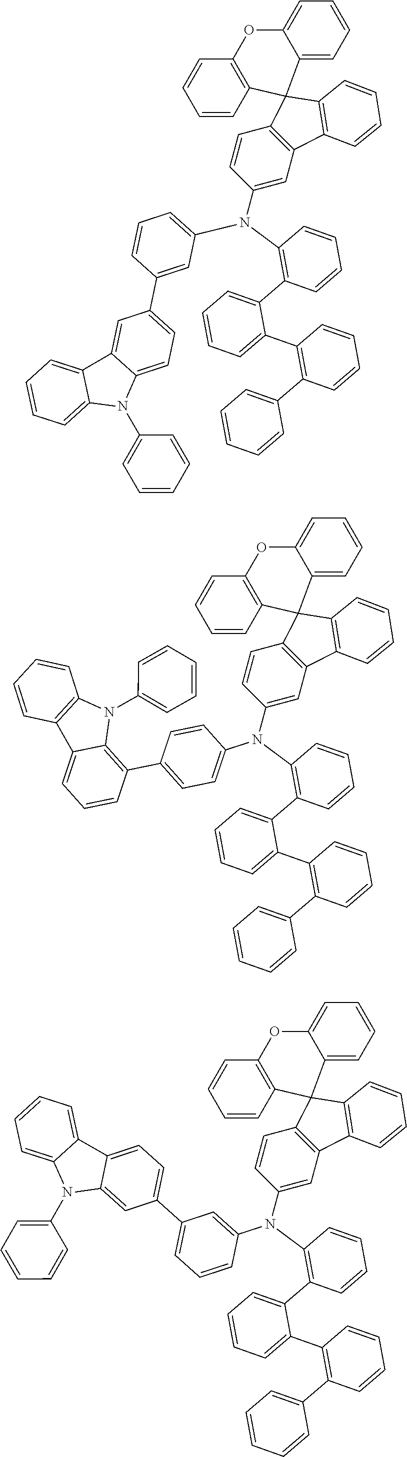 [SMILES: c1ccc(-c2ccccc2-c2ccccc2-c2ccccc2N(c2cccc(-c3ccc4c(c3)c3ccccc3n4-c3ccccc3)c2)c2ccc3c(c2)-c2ccccc2C32c3ccccc3Oc3ccccc32)cc1 ; c1ccc(-c2ccccc2-c2ccccc2-c2ccccc2N(c2ccc(-c3cccc4c5ccccc5n(-c5ccccc5)c34)cc2)c2ccc3c(c2)-c2ccccc2C32c3ccccc3Oc3ccccc32)cc1 ; c1ccc(-c2ccccc2-c2ccccc2-c2ccccc2N(c2cccc(-c3ccc4c5ccccc5n(-c5ccccc5)c4c3)c2)c2ccc3c(c2)-c2ccccc2C32c3ccccc3Oc3ccccc32)cc1]

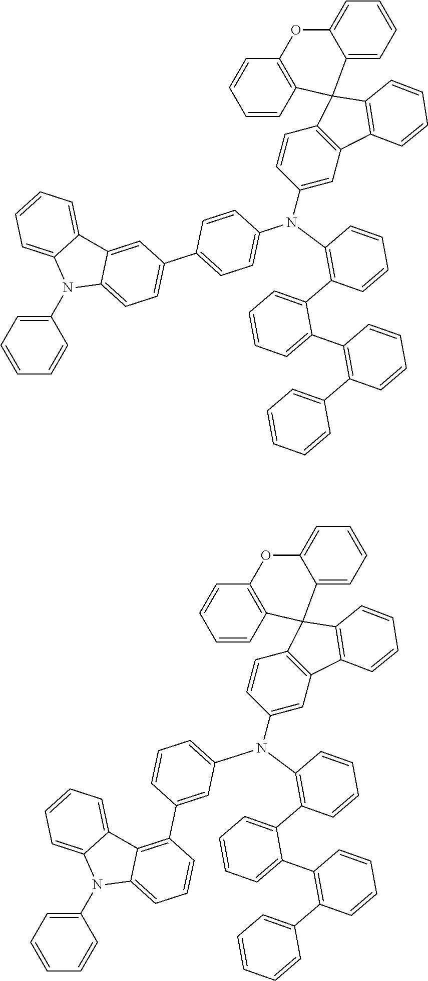 [SMILES: c1ccc(-c2ccccc2-c2ccccc2-c2ccccc2N(c2ccc(-c3ccc4c(c3)c3ccccc3n4-c3ccccc3)cc2)c2ccc3c(c2)-c2ccccc2C32c3ccccc3Oc3ccccc32)cc1 ; c1ccc(-c2ccccc2-c2ccccc2-c2ccccc2N(c2cccc(-c3cccc4c3c3ccccc3n4-c3ccccc3)c2)c2ccc3c(c2)-c2ccccc2C32c3ccccc3Oc3ccccc32)cc1]

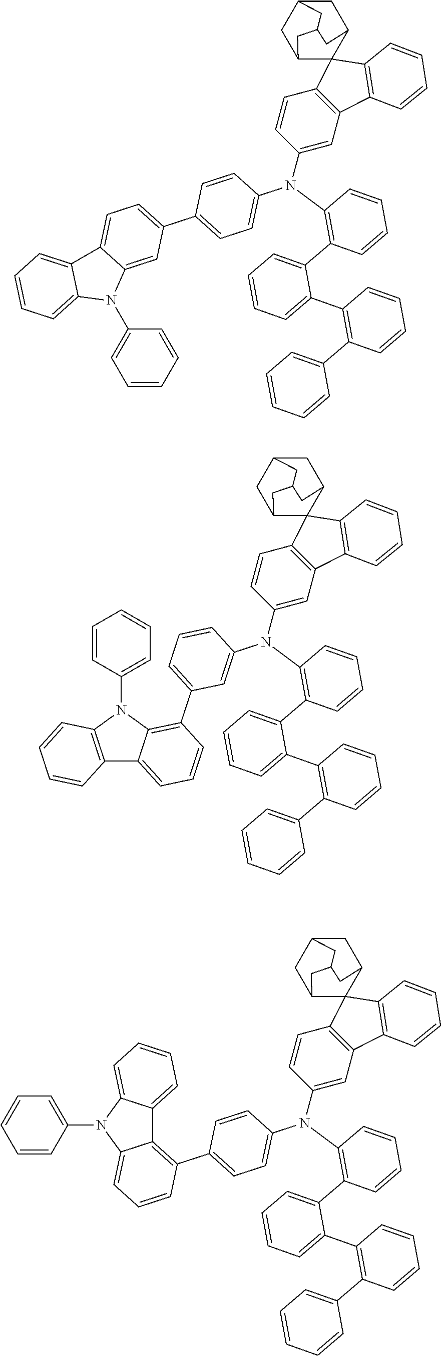

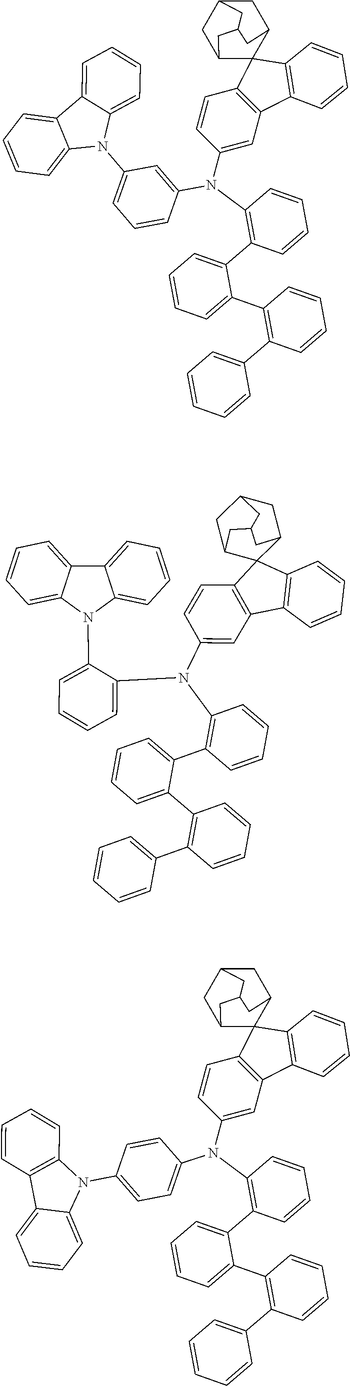 [SMILES: c1ccc(-c2ccccc2-c2ccccc2-c2ccccc2N(c2cccc(-n3c4ccccc4c4ccccc43)c2)c2ccc3c(c2)-c2ccccc2C32C3CC4CC(C3)CC2C4)cc1 ; c1ccc(-c2ccccc2-c2ccccc2-c2ccccc2N(c2ccc3c(c2)-c2ccccc2C32C3CC4CC(C3)CC2C4)c2ccccc2-n2c3ccccc3c3ccccc32)cc1 ; c1ccc(-c2ccccc2-c2ccccc2-c2ccccc2N(c2ccc(-n3c4ccccc4c4ccccc43)cc2)c2ccc3c(c2)-c2ccccc2C32C3CC4CC(C3)CC2C4)cc1]

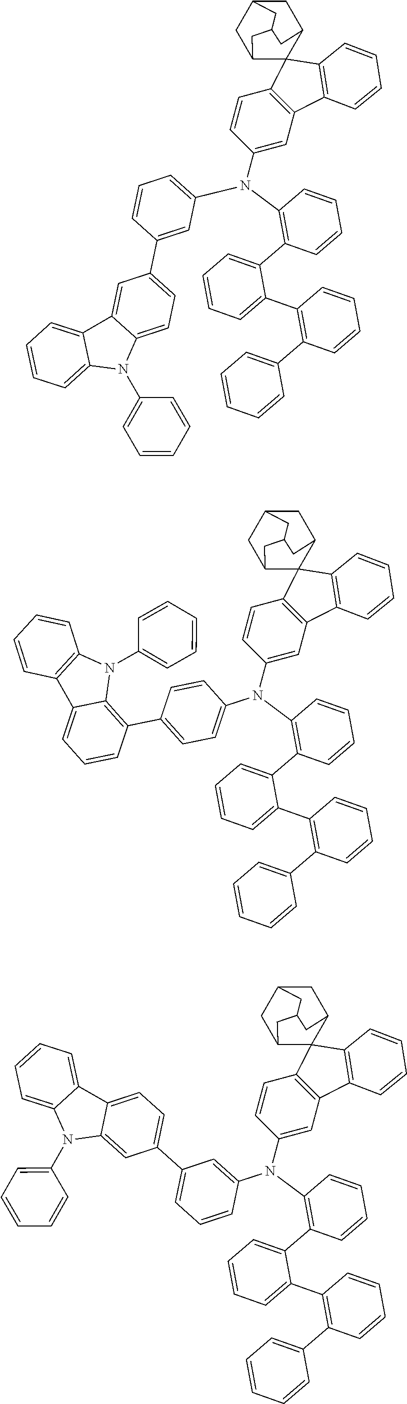 [SMILES: c1ccc(-c2ccccc2-c2ccccc2-c2ccccc2N(c2cccc(-c3ccc4c(c3)c3ccccc3n4-c3ccccc3)c2)c2ccc3c(c2)-c2ccccc2C32C3CC4CC(C3)CC2C4)cc1 ; c1ccc(-c2ccccc2-c2ccccc2-c2ccccc2N(c2ccc(-c3cccc4c5ccccc5n(-c5ccccc5)c34)cc2)c2ccc3c(c2)-c2ccccc2C32C3CC4CC(C3)CC2C4)cc1 ; c1ccc(-c2ccccc2-c2ccccc2-c2ccccc2N(c2cccc(-c3ccc4c5ccccc5n(-c5ccccc5)c4c3)c2)c2ccc3c(c2)-c2ccccc2C32C3CC4CC(C3)CC2C4)cc1]

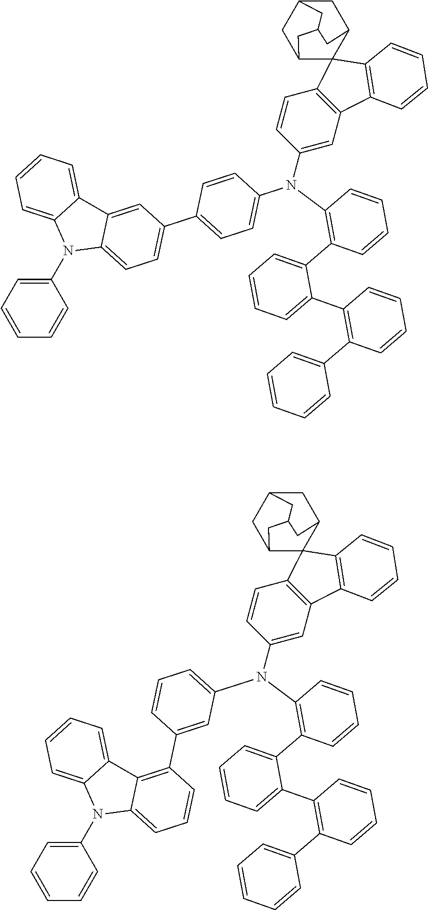 [SMILES: c1ccc(-c2ccccc2-c2ccccc2-c2ccccc2N(c2ccc(-c3ccc4c(c3)c3ccccc3n4-c3ccccc3)cc2)c2ccc3c(c2)-c2ccccc2C32C3CC4CC(C3)CC2C4)cc1 ; c1ccc(-c2ccccc2-c2ccccc2-c2ccccc2N(c2cccc(-c3cccc4c3c3ccccc3n4-c3ccccc3)c2)c2ccc3c(c2)-c2ccccc2C32C3CC4CC(C3)CC2C4)cc1]

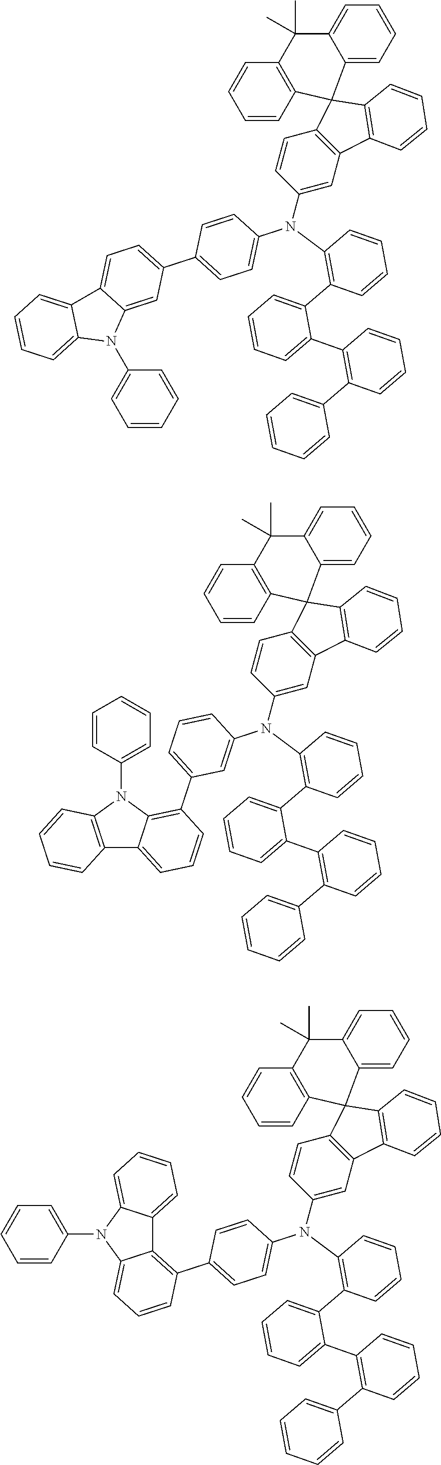 [SMILES: CC1(C)c2ccccc2C2(c3ccccc3-c3cc(N(c4ccc(-c5ccc6c7ccccc7n(-c7ccccc7)c6c5)cc4)c4ccccc4-c4ccccc4-c4ccccc4-c4ccccc4)ccc32)c2ccccc21 ; CC1(C)c2ccccc2C2(c3ccccc3-c3cc(N(c4cccc(-c5cccc6c7ccccc7n(-c7ccccc7)c56)c4)c4ccccc4-c4ccccc4-c4ccccc4-c4ccccc4)ccc32)c2ccccc21 ; CC1(C)c2ccccc2C2(c3ccccc3-c3cc(N(c4ccc(-c5cccc6c5c5ccccc5n6-c5ccccc5)cc4)c4ccccc4-c4ccccc4-c4ccccc4-c4ccccc4)ccc32)c2ccccc21]

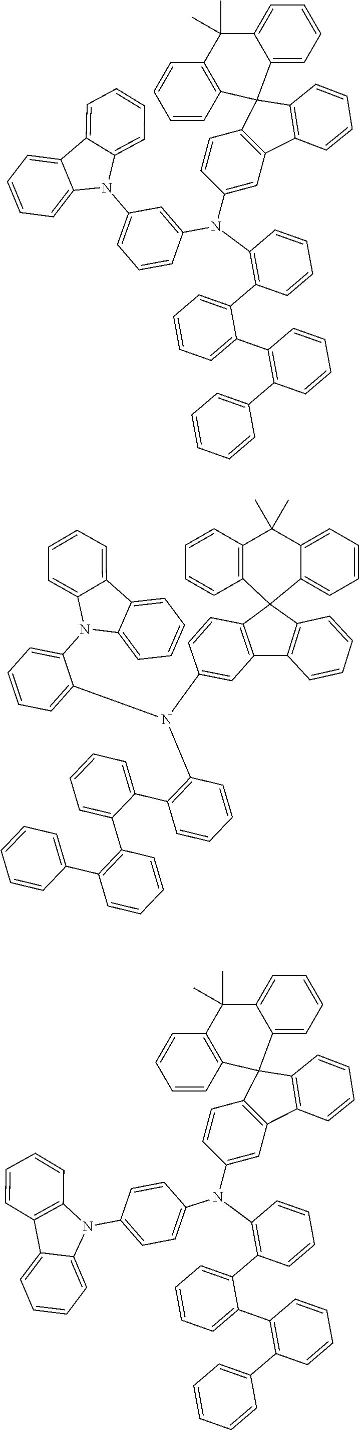 [SMILES: CC1(C)c2ccccc2C2(c3ccccc3-c3cc(N(c4cccc(-n5c6ccccc6c6ccccc65)c4)c4ccccc4-c4ccccc4-c4ccccc4-c4ccccc4)ccc32)c2ccccc21 ; CC1(C)c2ccccc2C2(c3ccccc3-c3cc(N(c4ccccc4-c4ccccc4-c4ccccc4-c4ccccc4)c4ccccc4-n4c5ccccc5c5ccccc54)ccc32)c2ccccc21 ; CC1(C)c2ccccc2C2(c3ccccc3-c3cc(N(c4ccc(-n5c6ccccc6c6ccccc65)cc4)c4ccccc4-c4ccccc4-c4ccccc4-c4ccccc4)ccc32)c2ccccc21]

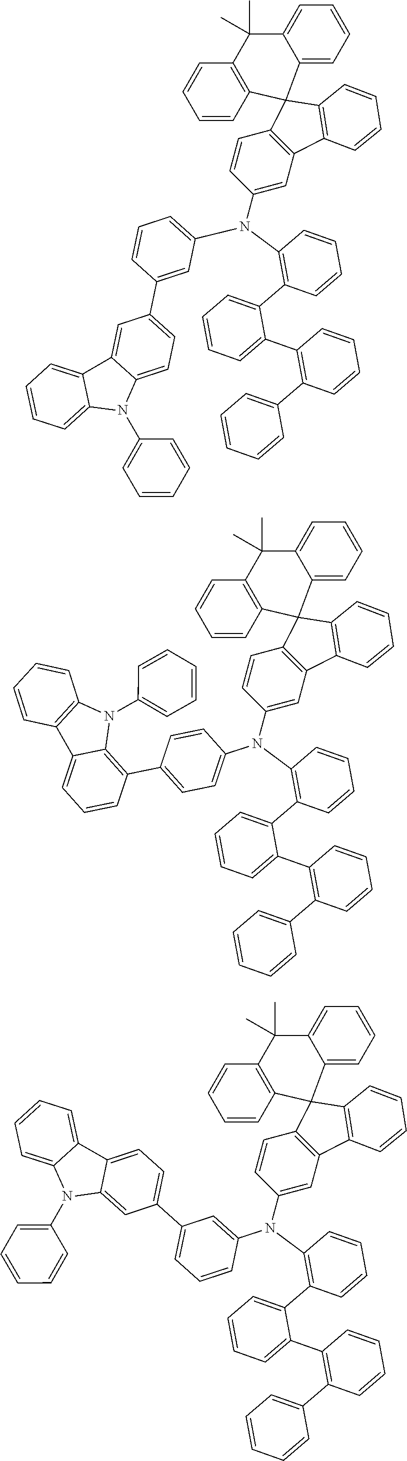

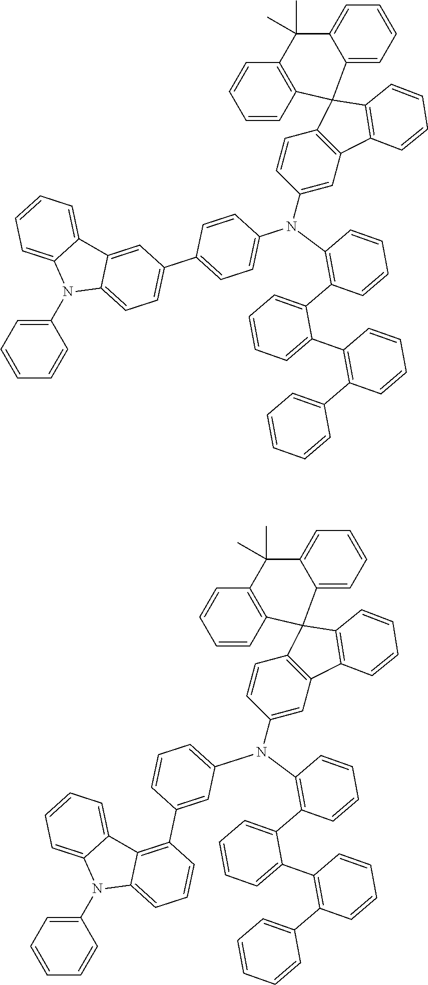 [SMILES: CC1(C)c2ccccc2C2(c3ccccc3-c3cc(N(c4ccc(-c5ccc6c(c5)c5ccccc5n6-c5ccccc5)cc4)c4ccccc4-c4ccccc4-c4ccccc4-c4ccccc4)ccc32)c2ccccc21 ; CC1(C)c2ccccc2C2(c3ccccc3-c3cc(N(c4cccc(-c5cccc6c5c5ccccc5n6-c5ccccc5)c4)c4ccccc4-c4ccccc4-c4ccccc4-c4ccccc4)ccc32)c2ccccc21]

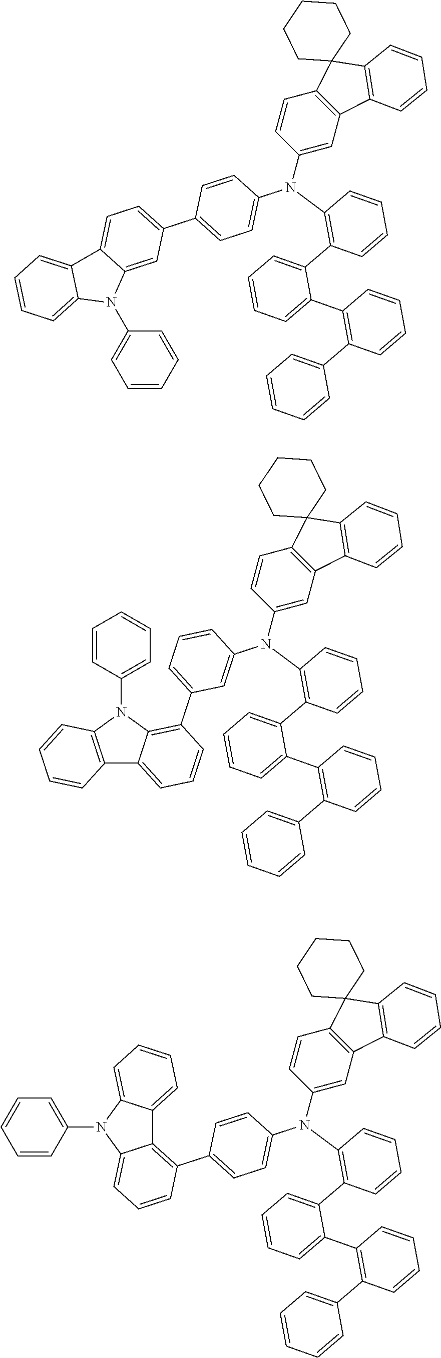

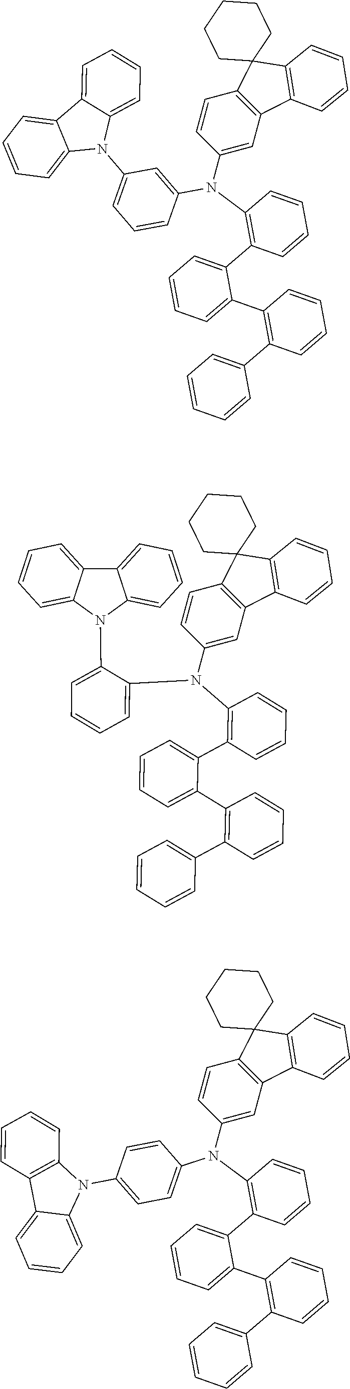 [SMILES: c1ccc(-c2ccccc2-c2ccccc2-c2ccccc2N(c2cccc(-n3c4ccccc4c4ccccc43)c2)c2ccc3c(c2)-c2ccccc2C32CCCCC2)cc1 ; c1ccc(-c2ccccc2-c2ccccc2-c2ccccc2N(c2ccc3c(c2)-c2ccccc2C32CCCCC2)c2ccccc2-n2c3ccccc3c3ccccc32)cc1 ; c1ccc(-c2ccccc2-c2ccccc2-c2ccccc2N(c2ccc(-n3c4ccccc4c4ccccc43)cc2)c2ccc3c(c2)-c2ccccc2C32CCCCC2)cc1]

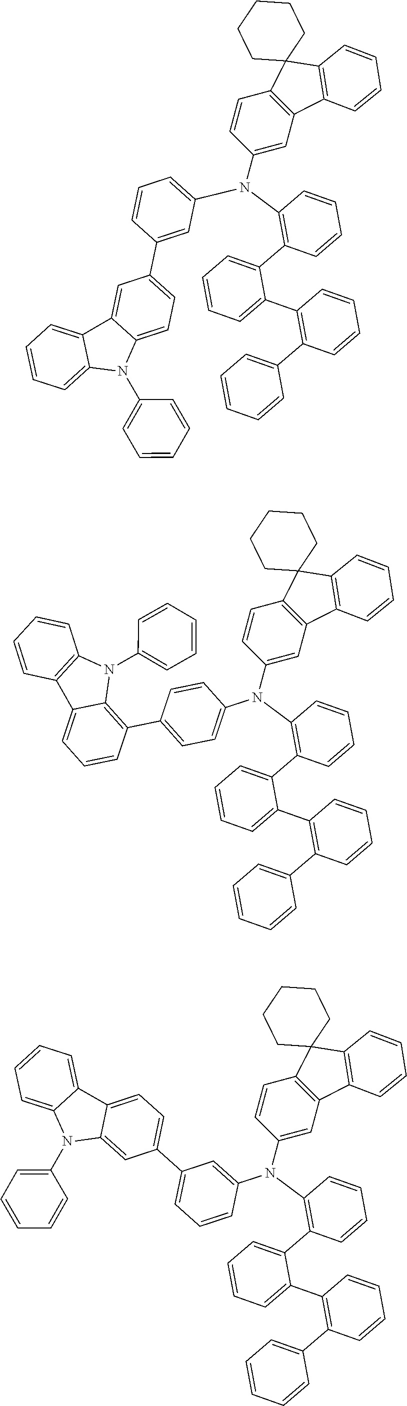 [SMILES: c1ccc(-c2ccccc2-c2ccccc2-c2ccccc2N(c2cccc(-c3ccc4c(c3)c3ccccc3n4-c3ccccc3)c2)c2ccc3c(c2)-c2ccccc2C32CCCCC2)cc1 ; c1ccc(-c2ccccc2-c2ccccc2-c2ccccc2N(c2ccc(-c3cccc4c5ccccc5n(-c5ccccc5)c34)cc2)c2ccc3c(c2)-c2ccccc2C32CCCCC2)cc1 ; c1ccc(-c2ccccc2-c2ccccc2-c2ccccc2N(c2cccc(-c3ccc4c5ccccc5n(-c5ccccc5)c4c3)c2)c2ccc3c(c2)-c2ccccc2C32CCCCC2)cc1]

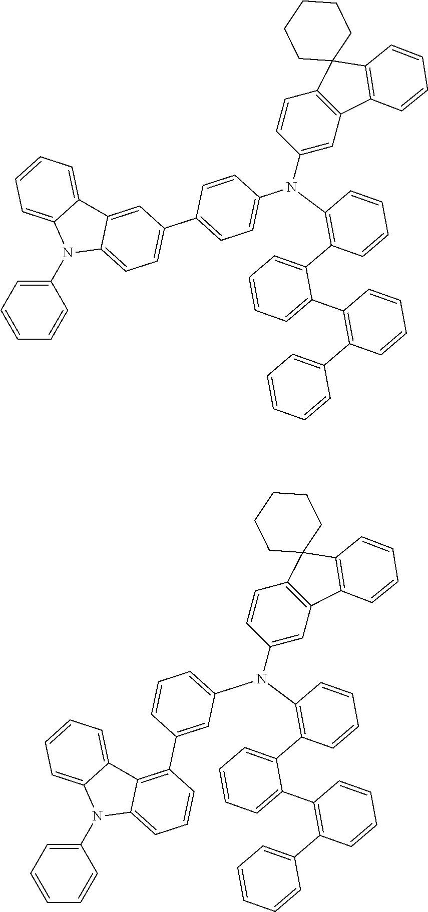 [SMILES: c1ccc(-c2ccccc2-c2ccccc2-c2ccccc2N(c2ccc(-c3ccc4c(c3)c3ccccc3n4-c3ccccc3)cc2)c2ccc3c(c2)-c2ccccc2C32CCCCC2)cc1 ; c1ccc(-c2ccccc2-c2ccccc2-c2ccccc2N(c2cccc(-c3cccc4c3c3ccccc3n4-c3ccccc3)c2)c2ccc3c(c2)-c2ccccc2C32CCCCC2)cc1]

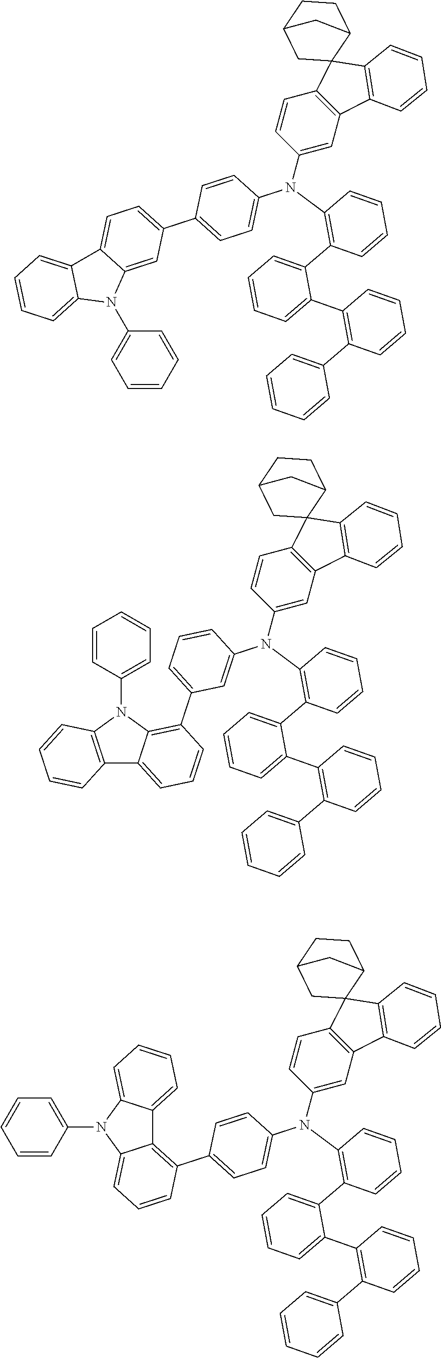

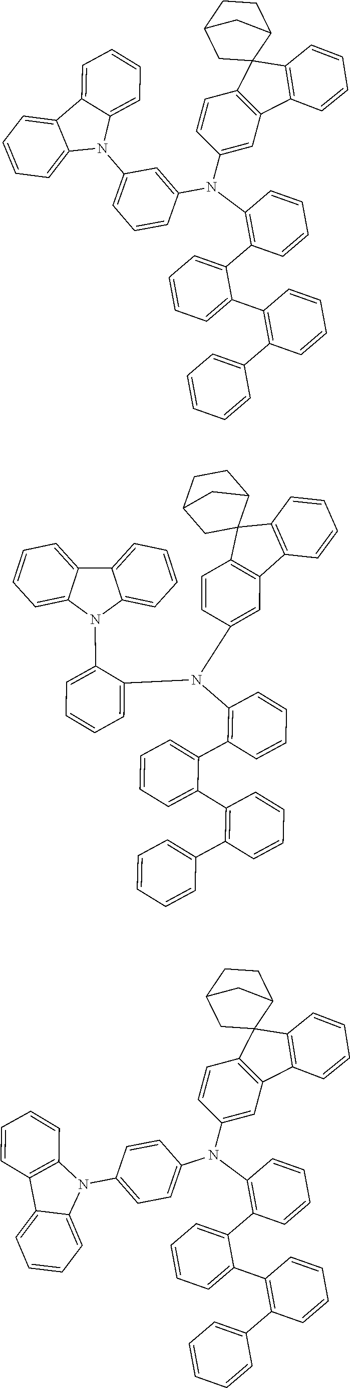 [SMILES: c1ccc(-c2ccccc2-c2ccccc2-c2ccccc2N(c2cccc(-n3c4ccccc4c4ccccc43)c2)c2ccc3c(c2)-c2ccccc2C32CC3CCC2C3)cc1 ; c1ccc(-c2ccccc2-c2ccccc2-c2ccccc2N(c2ccc3c(c2)-c2ccccc2C32CC3CCC2C3)c2ccccc2-n2c3ccccc3c3ccccc32)cc1 ; c1ccc(-c2ccccc2-c2ccccc2-c2ccccc2N(c2ccc(-n3c4ccccc4c4ccccc43)cc2)c2ccc3c(c2)-c2ccccc2C32CC3CCC2C3)cc1]

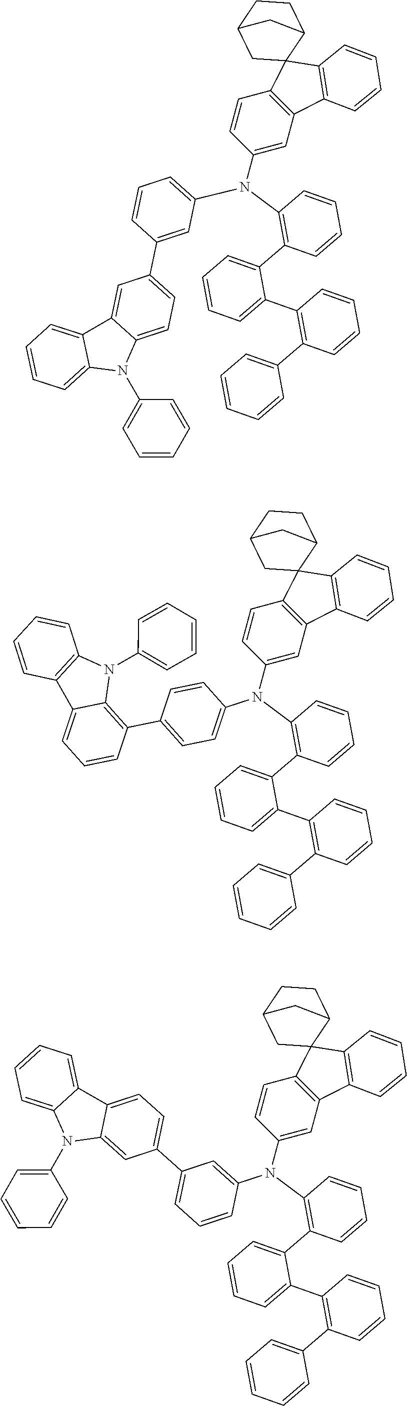 [SMILES: c1ccc(-c2ccccc2-c2ccccc2-c2ccccc2N(c2cccc(-c3ccc4c(c3)c3ccccc3n4-c3ccccc3)c2)c2ccc3c(c2)-c2ccccc2C32CC3CCC2C3)cc1 ; c1ccc(-c2ccccc2-c2ccccc2-c2ccccc2N(c2ccc(-c3cccc4c5ccccc5n(-c5ccccc5)c34)cc2)c2ccc3c(c2)-c2ccccc2C32CC3CCC2C3)cc1 ; c1ccc(-c2ccccc2-c2ccccc2-c2ccccc2N(c2cccc(-c3ccc4c5ccccc5n(-c5ccccc5)c4c3)c2)c2ccc3c(c2)-c2ccccc2C32CC3CCC2C3)cc1]

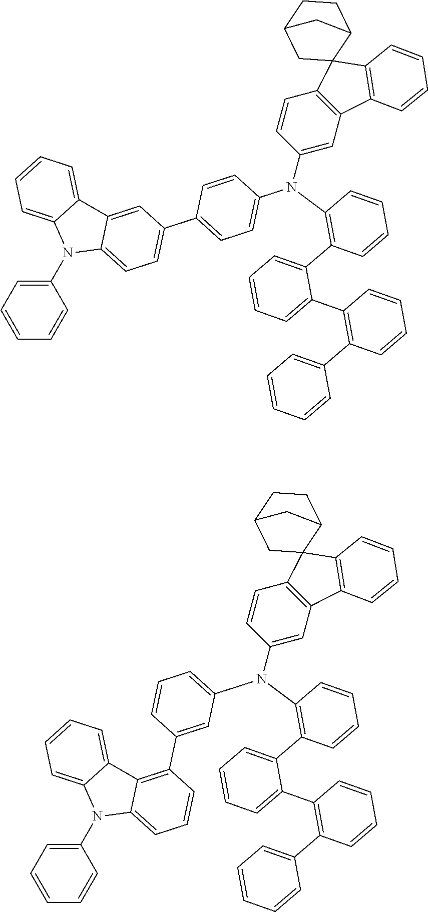 [SMILES: c1ccc(-c2ccccc2-c2ccccc2-c2ccccc2N(c2ccc(-c3ccc4c(c3)c3ccccc3n4-c3ccccc3)cc2)c2ccc3c(c2)-c2ccccc2C32CC3CCC2C3)cc1 ; c1ccc(-c2ccccc2-c2ccccc2-c2ccccc2N(c2cccc(-c3cccc4c3c3ccccc3n4-c3ccccc3)c2)c2ccc3c(c2)-c2ccccc2C32CC3CCC2C3)cc1]

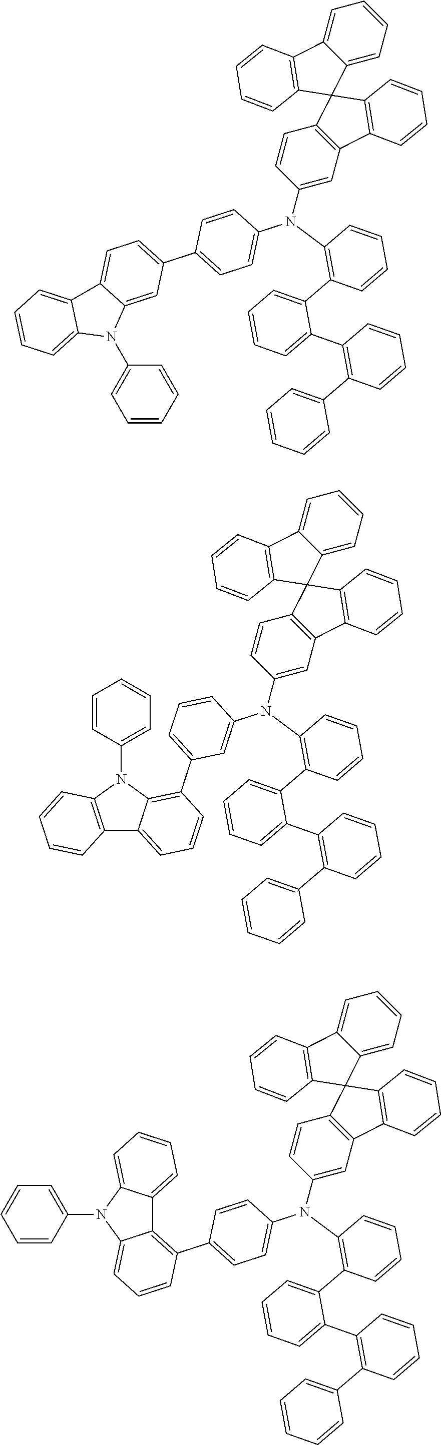

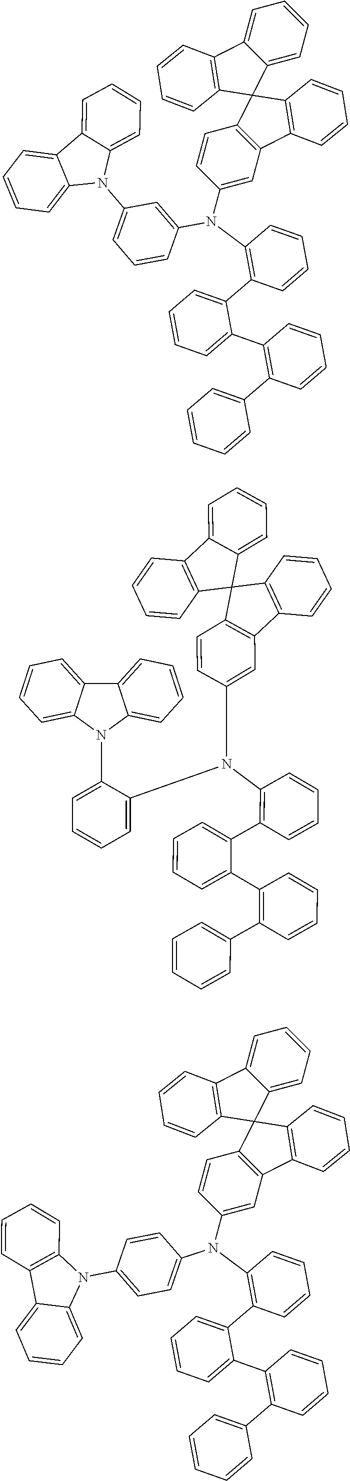

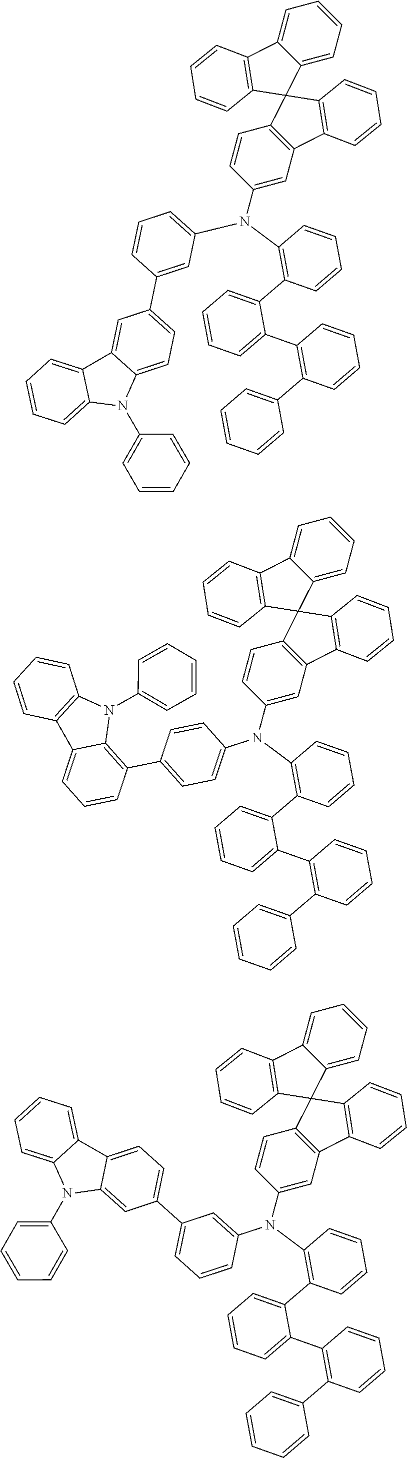 [SMILES: c1ccc(-c2ccccc2-c2ccccc2-c2ccccc2N(c2cccc(-c3ccc4c(c3)c3ccccc3n4-c3ccccc3)c2)c2ccc3c(c2)-c2ccccc2C32c3ccccc3-c3ccccc32)cc1 ; c1ccc(-c2ccccc2-c2ccccc2-c2ccccc2N(c2ccc(-c3cccc4c5ccccc5n(-c5ccccc5)c34)cc2)c2ccc3c(c2)-c2ccccc2C32c3ccccc3-c3ccccc32)cc1 ; c1ccc(-c2ccccc2-c2ccccc2-c2ccccc2N(c2cccc(-c3ccc4c5ccccc5n(-c5ccccc5)c4c3)c2)c2ccc3c(c2)-c2ccccc2C32c3ccccc3-c3ccccc32)cc1]

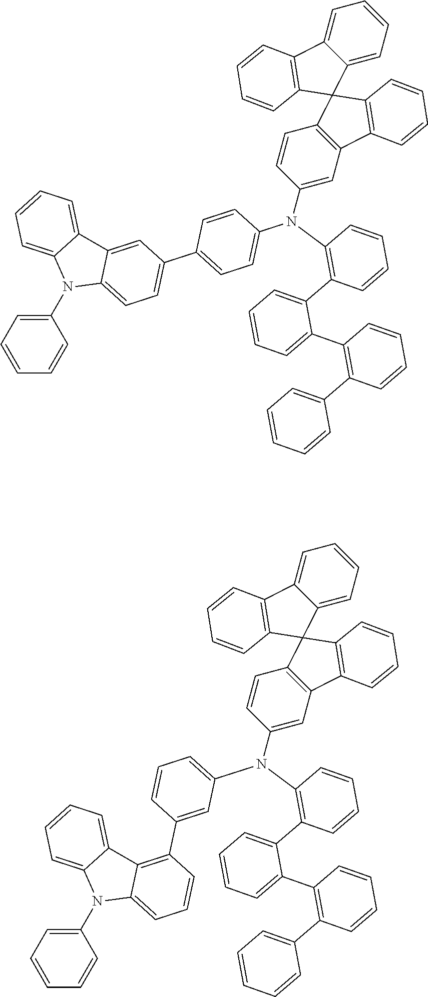 [SMILES: c1ccc(-c2ccccc2-c2ccccc2-c2ccccc2N(c2ccc(-c3ccc4c(c3)c3ccccc3n4-c3ccccc3)cc2)c2ccc3c(c2)-c2ccccc2C32c3ccccc3-c3ccccc32)cc1 ; c1ccc(-c2ccccc2-c2ccccc2-c2ccccc2N(c2cccc(-c3cccc4c3c3ccccc3n4-c3ccccc3)c2)c2ccc3c(c2)-c2ccccc2C32c3ccccc3-c3ccccc32)cc1]

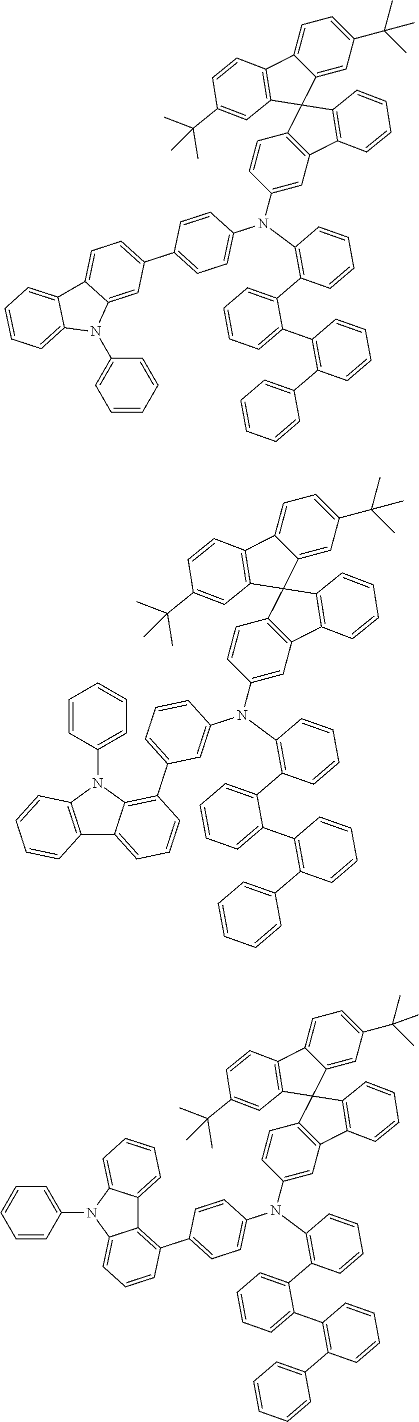 [SMILES: CC(C)(C)c1ccc2c(c1)C1(c3ccccc3-c3cc(N(c4ccc(-c5ccc6c7ccccc7n(-c7ccccc7)c6c5)cc4)c4ccccc4-c4ccccc4-c4ccccc4-c4ccccc4)ccc31)c1cc(C(C)(C)C)ccc1-2 ; CC(C)(C)c1ccc2c(c1)C1(c3ccccc3-c3cc(N(c4cccc(-c5cccc6c7ccccc7n(-c7ccccc7)c56)c4)c4ccccc4-c4ccccc4-c4ccccc4-c4ccccc4)ccc31)c1cc(C(C)(C)C)ccc1-2 ; CC(C)(C)c1ccc2c(c1)C1(c3ccccc3-c3cc(N(c4ccc(-c5cccc6c5c5ccccc5n6-c5ccccc5)cc4)c4ccccc4-c4ccccc4-c4ccccc4-c4ccccc4)ccc31)c1cc(C(C)(C)C)ccc1-2]

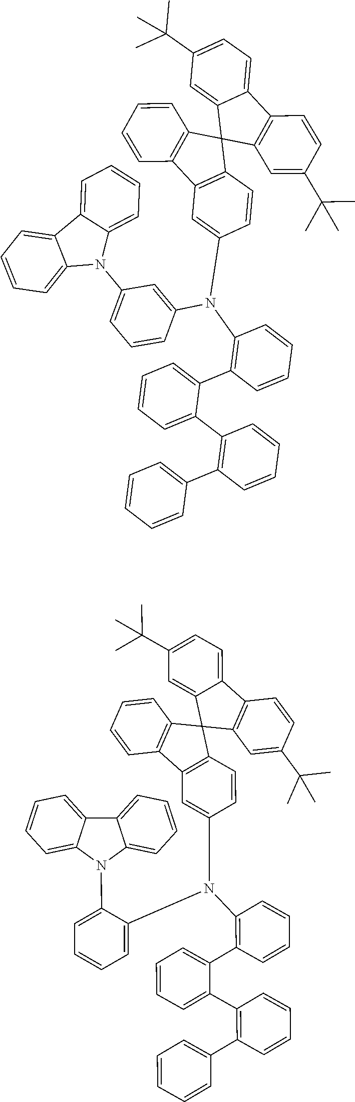 [SMILES: CC(C)(C)c1ccc2c(c1)C1(c3ccccc3-c3cc(N(c4cccc(-n5c6ccccc6c6ccccc65)c4)c4ccccc4-c4ccccc4-c4ccccc4-c4ccccc4)ccc31)c1cc(C(C)(C)C)ccc1-2 ; CC(C)(C)c1ccc2c(c1)C1(c3ccccc3-c3cc(N(c4ccccc4-c4ccccc4-c4ccccc4-c4ccccc4)c4ccccc4-n4c5ccccc5c5ccccc54)ccc31)c1cc(C(C)(C)C)ccc1-2]

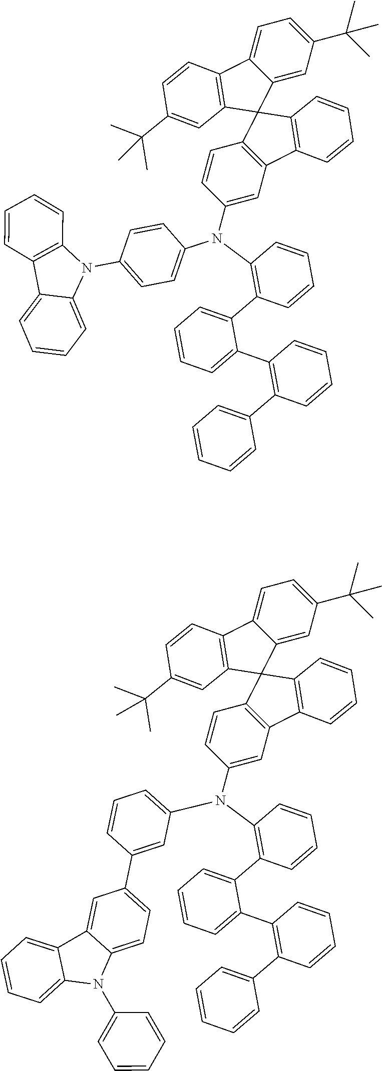 [SMILES: CC(C)(C)c1ccc2c(c1)C1(c3ccccc3-c3cc(N(c4ccc(-n5c6ccccc6c6ccccc65)cc4)c4ccccc4-c4ccccc4-c4ccccc4-c4ccccc4)ccc31)c1cc(C(C)(C)C)ccc1-2 ; CC(C)(C)c1ccc2c(c1)C1(c3ccccc3-c3cc(N(c4cccc(-c5ccc6c(c5)c5ccccc5n6-c5ccccc5)c4)c4ccccc4-c4ccccc4-c4ccccc4-c4ccccc4)ccc31)c1cc(C(C)(C)C)ccc1-2]

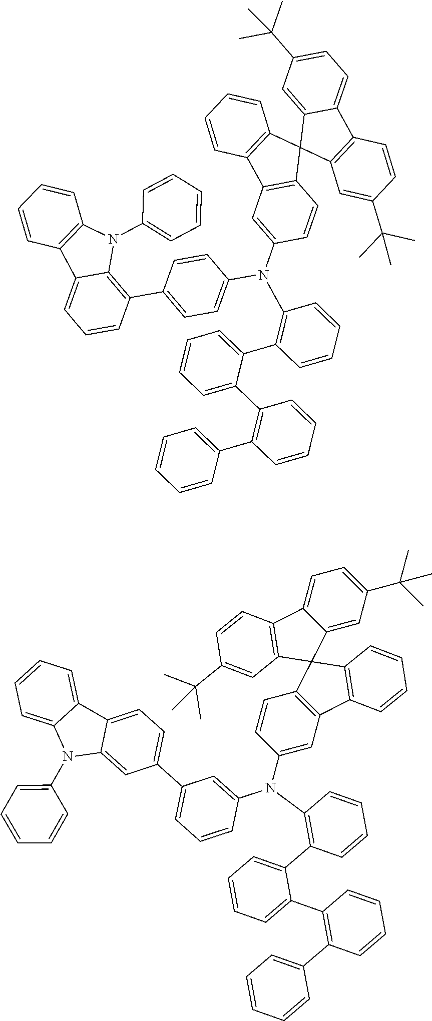 [SMILES: CC(C)(C)c1ccc2c(c1)C1(c3ccccc3-c3cc(N(c4ccc(-c5cccc6c7ccccc7n(-c7ccccc7)c56)cc4)c4ccccc4-c4ccccc4-c4ccccc4-c4ccccc4)ccc31)c1cc(C(C)(C)C)ccc1-2 ; CC(C)(C)c1ccc2c(c1)C1(c3ccccc3-c3cc(N(c4cccc(-c5ccc6c7ccccc7n(-c7ccccc7)c6c5)c4)c4ccccc4-c4ccccc4-c4ccccc4-c4ccccc4)ccc31)c1cc(C(C)(C)C)ccc1-2]

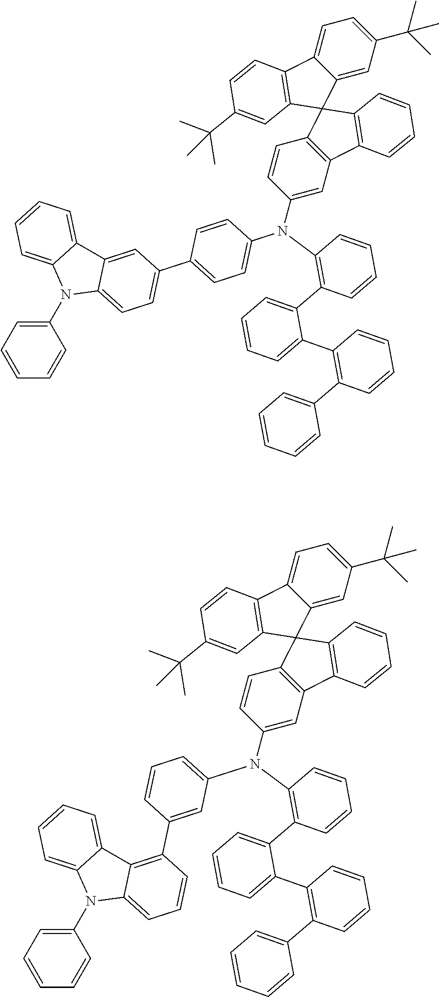 [SMILES: CC(C)(C)c1ccc2c(c1)C1(c3ccccc3-c3cc(N(c4ccc(-c5ccc6c(c5)c5ccccc5n6-c5ccccc5)cc4)c4ccccc4-c4ccccc4-c4ccccc4-c4ccccc4)ccc31)c1cc(C(C)(C)C)ccc1-2 ; CC(C)(C)c1ccc2c(c1)C1(c3ccccc3-c3cc(N(c4cccc(-c5cccc6c5c5ccccc5n6-c5ccccc5)c4)c4ccccc4-c4ccccc4-c4ccccc4-c4ccccc4)ccc31)c1cc(C(C)(C)C)ccc1-2]

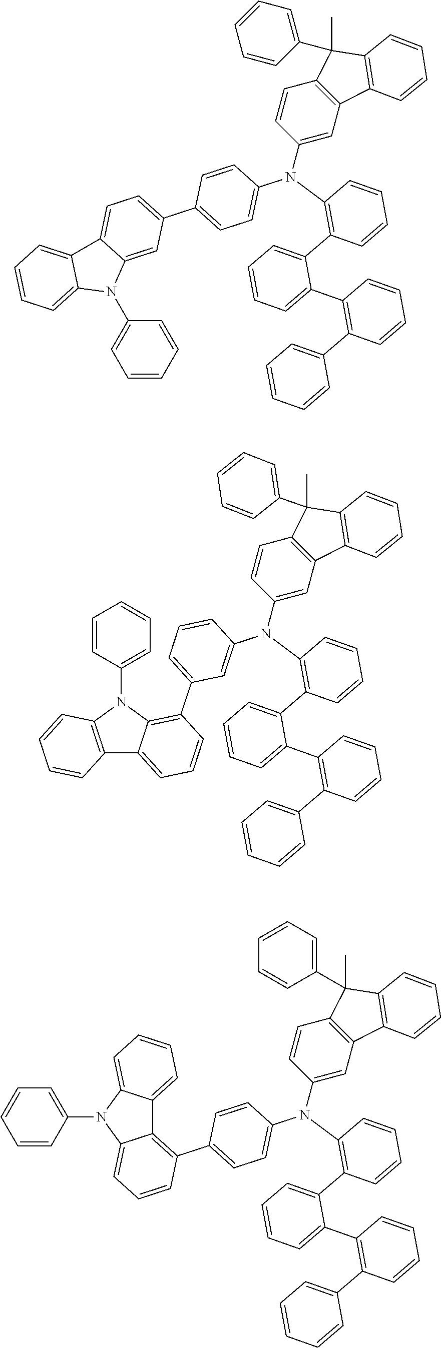 [SMILES: CC1(c2ccccc2)c2ccccc2-c2cc(N(c3ccc(-c4ccc5c6ccccc6n(-c6ccccc6)c5c4)cc3)c3ccccc3-c3ccccc3-c3ccccc3-c3ccccc3)ccc21 ; CC1(c2ccccc2)c2ccccc2-c2cc(N(c3cccc(-c4cccc5c6ccccc6n(-c6ccccc6)c45)c3)c3ccccc3-c3ccccc3-c3ccccc3-c3ccccc3)ccc21 ; CC1(c2ccccc2)c2ccccc2-c2cc(N(c3ccc(-c4cccc5c4c4ccccc4n5-c4ccccc4)cc3)c3ccccc3-c3ccccc3-c3ccccc3-c3ccccc3)ccc21]

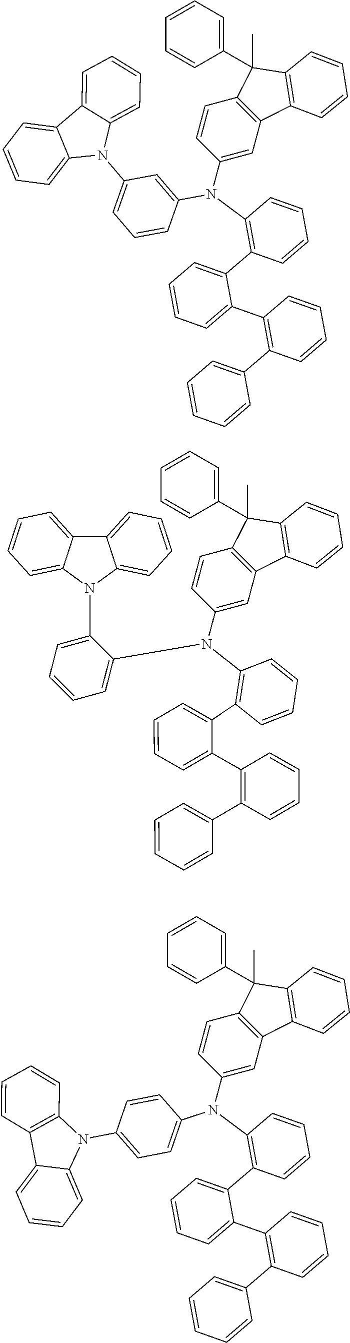

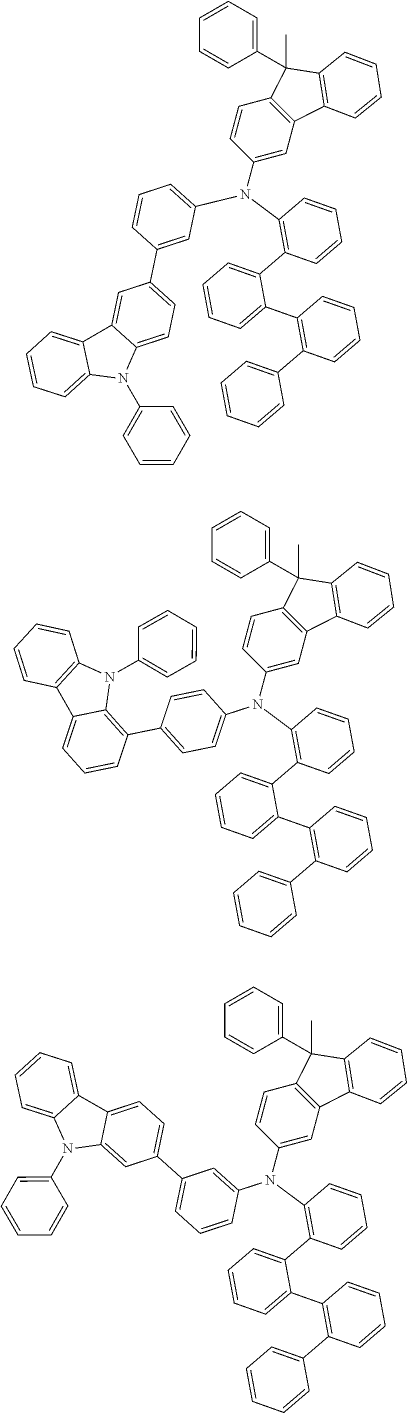 [SMILES: CC1(c2ccccc2)c2ccccc2-c2cc(N(c3cccc(-c4ccc5c(c4)c4ccccc4n5-c4ccccc4)c3)c3ccccc3-c3ccccc3-c3ccccc3-c3ccccc3)ccc21 ; CC1(c2ccccc2)c2ccccc2-c2cc(N(c3ccc(-c4cccc5c6ccccc6n(-c6ccccc6)c45)cc3)c3ccccc3-c3ccccc3-c3ccccc3-c3ccccc3)ccc21 ; CC1(c2ccccc2)c2ccccc2-c2cc(N(c3cccc(-c4ccc5c6ccccc6n(-c6ccccc6)c5c4)c3)c3ccccc3-c3ccccc3-c3ccccc3-c3ccccc3)ccc21]

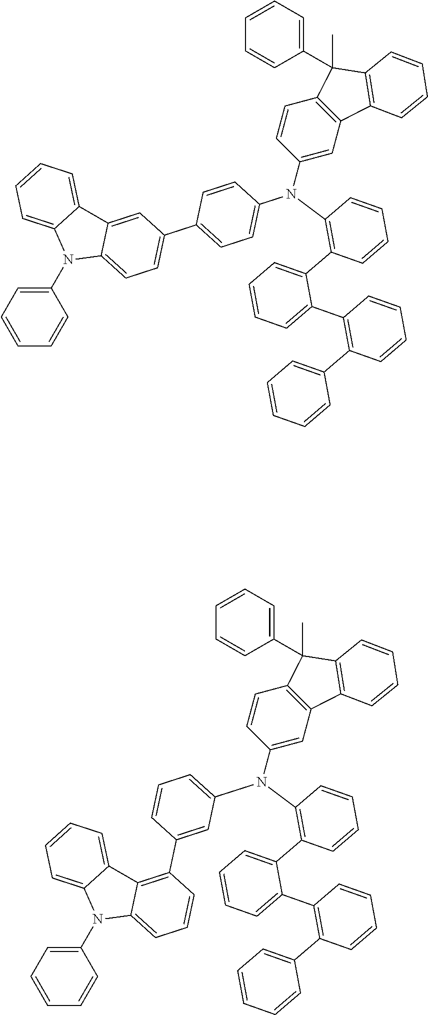 [SMILES: CC1(c2ccccc2)c2ccccc2-c2cc(N(c3ccc(-c4ccc5c(c4)c4ccccc4n5-c4ccccc4)cc3)c3ccccc3-c3ccccc3-c3ccccc3-c3ccccc3)ccc21 ; CC1(c2ccccc2)c2ccccc2-c2cc(N(c3cccc(-c4cccc5c4c4ccccc4n5-c4ccccc4)c3)c3ccccc3-c3ccccc3-c3ccccc3-c3ccccc3)ccc21]

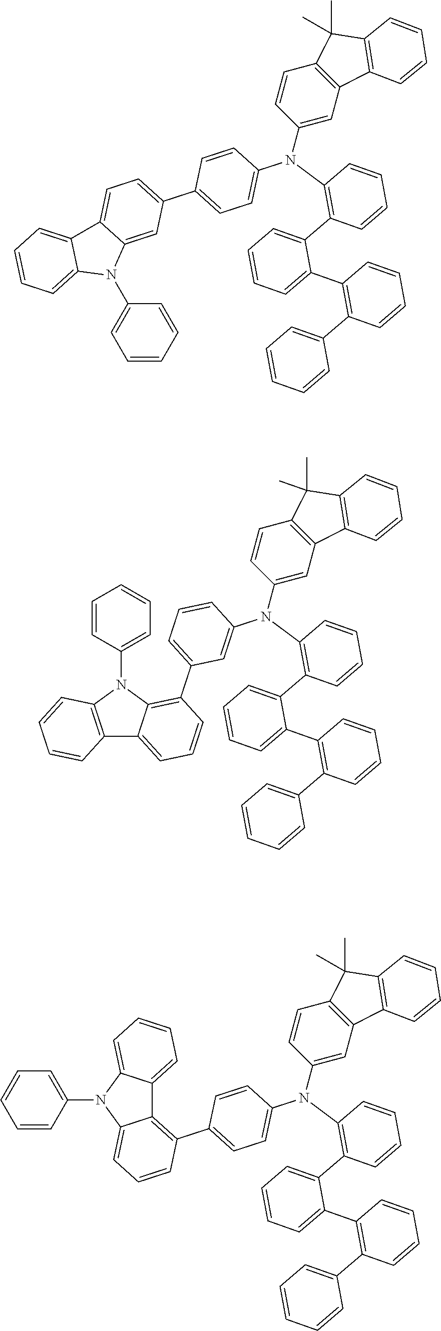 [SMILES: CC1(C)c2ccccc2-c2cc(N(c3ccc(-c4ccc5c6ccccc6n(-c6ccccc6)c5c4)cc3)c3ccccc3-c3ccccc3-c3ccccc3-c3ccccc3)ccc21 ; CC1(C)c2ccccc2-c2cc(N(c3cccc(-c4cccc5c6ccccc6n(-c6ccccc6)c45)c3)c3ccccc3-c3ccccc3-c3ccccc3-c3ccccc3)ccc21 ; CC1(C)c2ccccc2-c2cc(N(c3ccc(-c4cccc5c4c4ccccc4n5-c4ccccc4)cc3)c3ccccc3-c3ccccc3-c3ccccc3-c3ccccc3)ccc21]

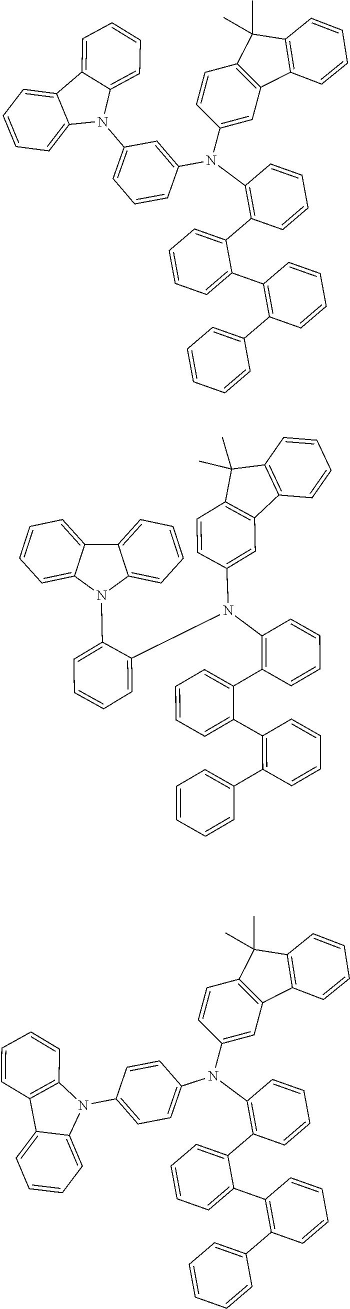

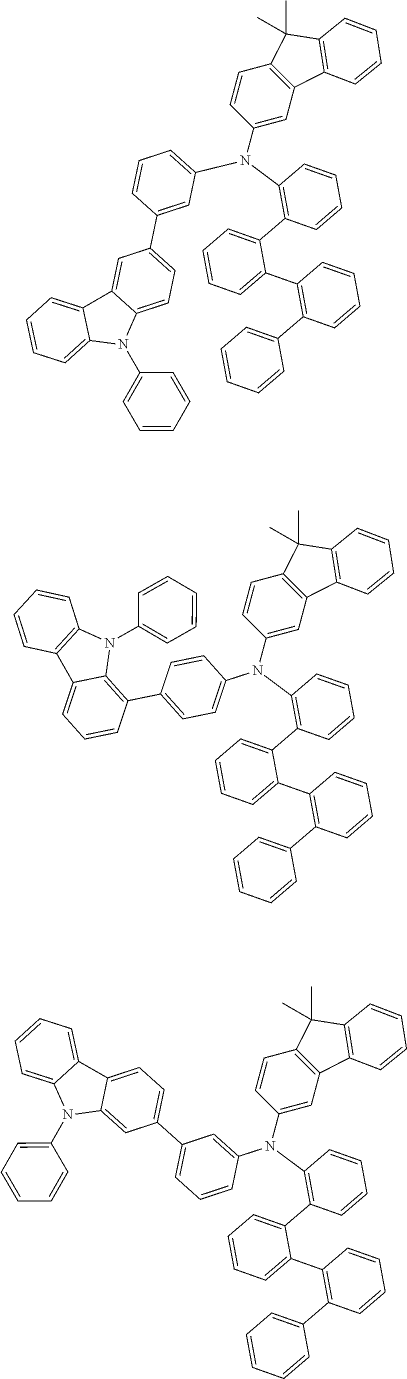 [SMILES: CC1(C)c2ccccc2-c2cc(N(c3cccc(-c4ccc5c(c4)c4ccccc4n5-c4ccccc4)c3)c3ccccc3-c3ccccc3-c3ccccc3-c3ccccc3)ccc21 ; CC1(C)c2ccccc2-c2cc(N(c3ccc(-c4cccc5c6ccccc6n(-c6ccccc6)c45)cc3)c3ccccc3-c3ccccc3-c3ccccc3-c3ccccc3)ccc21 ; CC1(C)c2ccccc2-c2cc(N(c3cccc(-c4ccc5c6ccccc6n(-c6ccccc6)c5c4)c3)c3ccccc3-c3ccccc3-c3ccccc3-c3ccccc3)ccc21]

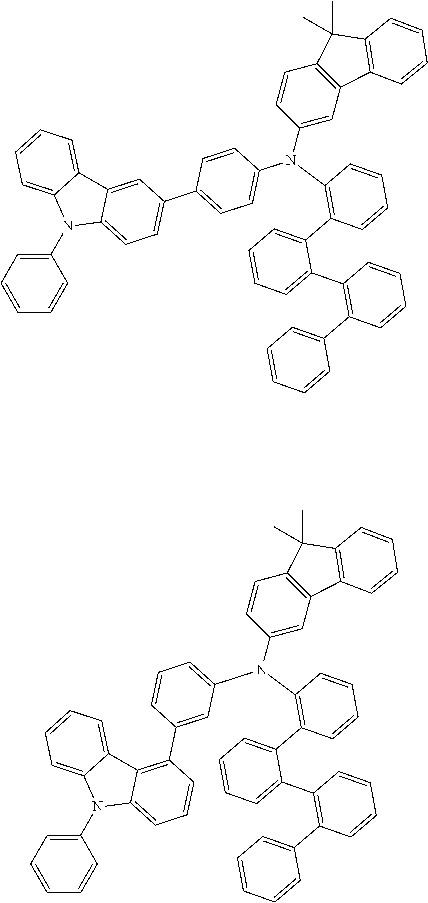 [SMILES: CC1(C)c2ccccc2-c2cc(N(c3ccc(-c4ccc5c(c4)c4ccccc4n5-c4ccccc4)cc3)c3ccccc3-c3ccccc3-c3ccccc3-c3ccccc3)ccc21 ; CC1(C)c2ccccc2-c2cc(N(c3cccc(-c4cccc5c4c4ccccc4n5-c4ccccc4)c3)c3ccccc3-c3ccccc3-c3ccccc3-c3ccccc3)ccc21]

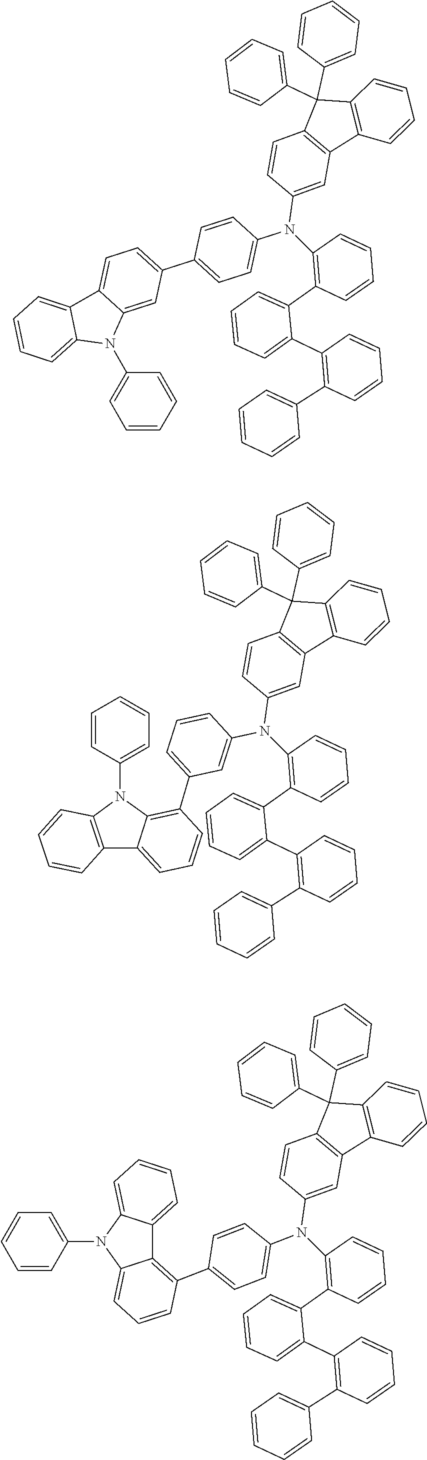 [SMILES: c1ccc(-c2ccccc2-c2ccccc2-c2ccccc2N(c2ccc(-c3ccc4c5ccccc5n(-c5ccccc5)c4c3)cc2)c2ccc3c(c2)-c2ccccc2C3(c2ccccc2)c2ccccc2)cc1 ; c1ccc(-c2ccccc2-c2ccccc2-c2ccccc2N(c2cccc(-c3cccc4c5ccccc5n(-c5ccccc5)c34)c2)c2ccc3c(c2)-c2ccccc2C3(c2ccccc2)c2ccccc2)cc1 ; c1ccc(-c2ccccc2-c2ccccc2-c2ccccc2N(c2ccc(-c3cccc4c3c3ccccc3n4-c3ccccc3)cc2)c2ccc3c(c2)-c2ccccc2C3(c2ccccc2)c2ccccc2)cc1]

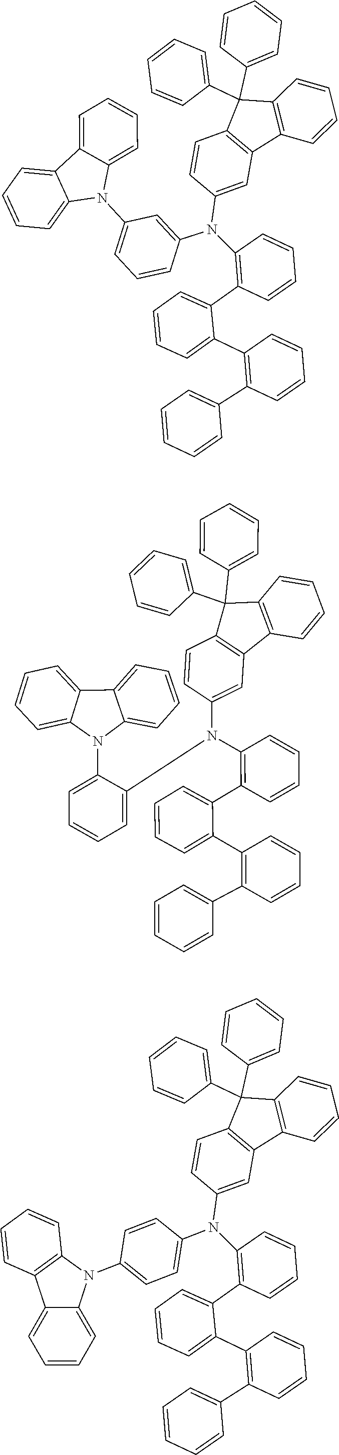 [SMILES: c1ccc(-c2ccccc2-c2ccccc2-c2ccccc2N(c2cccc(-n3c4ccccc4c4ccccc43)c2)c2ccc3c(c2)-c2ccccc2C3(c2ccccc2)c2ccccc2)cc1 ; c1ccc(-c2ccccc2-c2ccccc2-c2ccccc2N(c2ccc3c(c2)-c2ccccc2C3(c2ccccc2)c2ccccc2)c2ccccc2-n2c3ccccc3c3ccccc32)cc1 ; c1ccc(-c2ccccc2-c2ccccc2-c2ccccc2N(c2ccc(-n3c4ccccc4c4ccccc43)cc2)c2ccc3c(c2)-c2ccccc2C3(c2ccccc2)c2ccccc2)cc1]

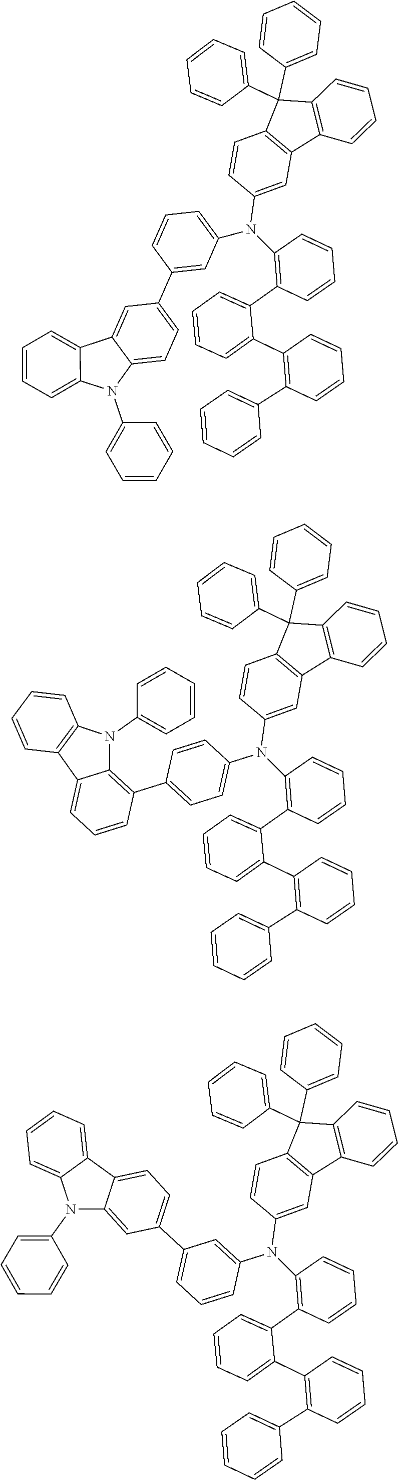 [SMILES: c1ccc(-c2ccccc2-c2ccccc2-c2ccccc2N(c2cccc(-c3ccc4c(c3)c3ccccc3n4-c3ccccc3)c2)c2ccc3c(c2)-c2ccccc2C3(c2ccccc2)c2ccccc2)cc1 ; c1ccc(-c2ccccc2-c2ccccc2-c2ccccc2N(c2ccc(-c3cccc4c5ccccc5n(-c5ccccc5)c34)cc2)c2ccc3c(c2)-c2ccccc2C3(c2ccccc2)c2ccccc2)cc1 ; c1ccc(-c2ccccc2-c2ccccc2-c2ccccc2N(c2cccc(-c3ccc4c5ccccc5n(-c5ccccc5)c4c3)c2)c2ccc3c(c2)-c2ccccc2C3(c2ccccc2)c2ccccc2)cc1]

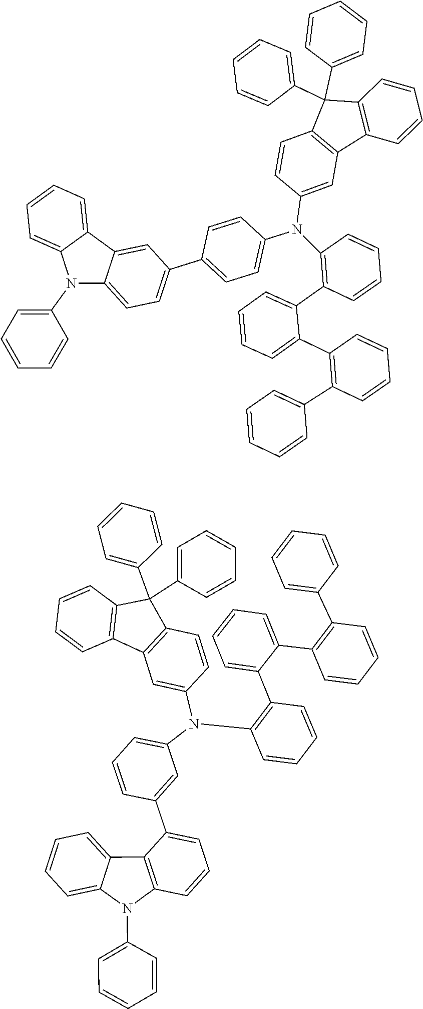 [SMILES: c1ccc(-c2ccccc2-c2ccccc2-c2ccccc2N(c2ccc(-c3ccc4c(c3)c3ccccc3n4-c3ccccc3)cc2)c2ccc3c(c2)-c2ccccc2C3(c2ccccc2)c2ccccc2)cc1 ; c1ccc(-c2ccccc2-c2ccccc2-c2ccccc2N(c2cccc(-c3cccc4c3c3ccccc3n4-c3ccccc3)c2)c2ccc3c(c2)-c2ccccc2C3(c2ccccc2)c2ccccc2)cc1]

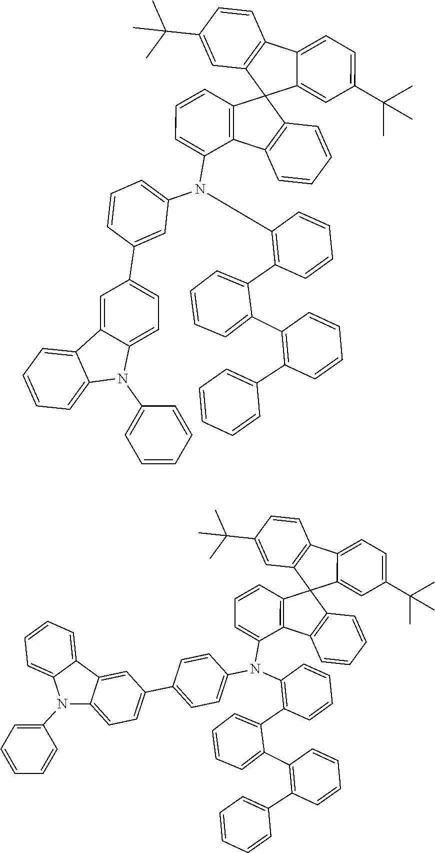 [SMILES: CC(C)(C)c1ccc2c(c1)C1(c3cc(C(C)(C)C)ccc3-2)c2ccccc2-c2c(N(c3cccc(-c4ccc5c(c4)c4ccccc4n5-c4ccccc4)c3)c3ccccc3-c3ccccc3-c3ccccc3-c3ccccc3)cccc21 ; CC(C)(C)c1ccc2c(c1)C1(c3cc(C(C)(C)C)ccc3-2)c2ccccc2-c2c(N(c3ccc(-c4ccc5c(c4)c4ccccc4n5-c4ccccc4)cc3)c3ccccc3-c3ccccc3-c3ccccc3-c3ccccc3)cccc21]

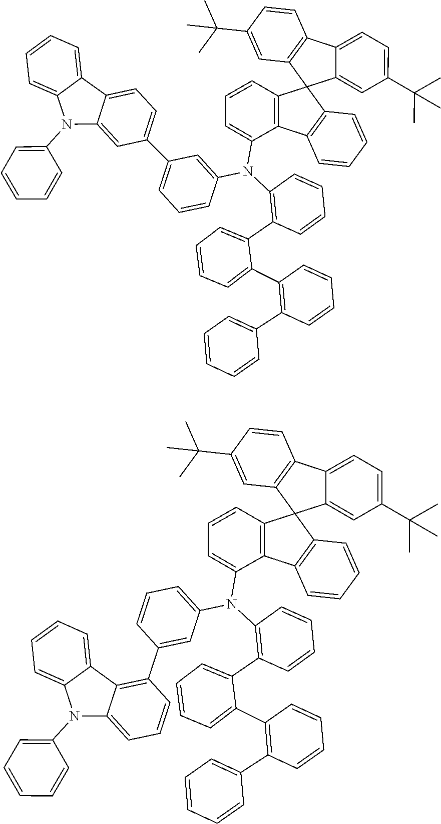 [SMILES: CC(C)(C)c1ccc2c(c1)C1(c3cc(C(C)(C)C)ccc3-2)c2ccccc2-c2c(N(c3cccc(-c4ccc5c6ccccc6n(-c6ccccc6)c5c4)c3)c3ccccc3-c3ccccc3-c3ccccc3-c3ccccc3)cccc21 ; CC(C)(C)c1ccc2c(c1)C1(c3cc(C(C)(C)C)ccc3-2)c2ccccc2-c2c(N(c3cccc(-c4cccc5c4c4ccccc4n5-c4ccccc4)c3)c3ccccc3-c3ccccc3-c3ccccc3-c3ccccc3)cccc21]

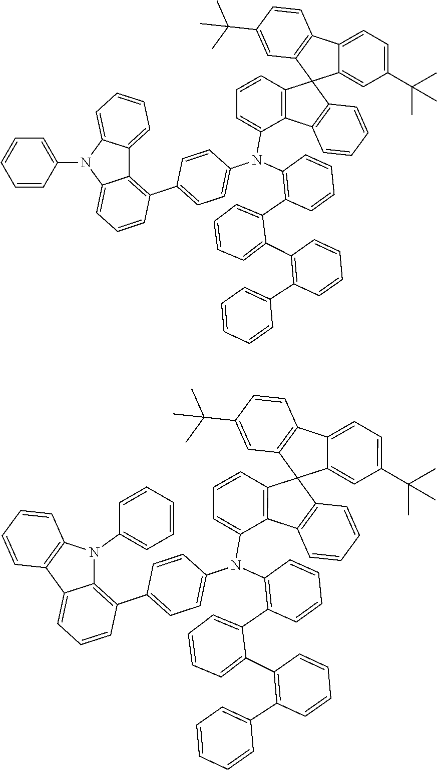 [SMILES: CC(C)(C)c1ccc2c(c1)C1(c3cc(C(C)(C)C)ccc3-2)c2ccccc2-c2c(N(c3ccc(-c4cccc5c4c4ccccc4n5-c4ccccc4)cc3)c3ccccc3-c3ccccc3-c3ccccc3-c3ccccc3)cccc21 ; CC(C)(C)c1ccc2c(c1)C1(c3cc(C(C)(C)C)ccc3-2)c2ccccc2-c2c(N(c3ccc(-c4cccc5c6ccccc6n(-c6ccccc6)c45)cc3)c3ccccc3-c3ccccc3-c3ccccc3-c3ccccc3)cccc21]

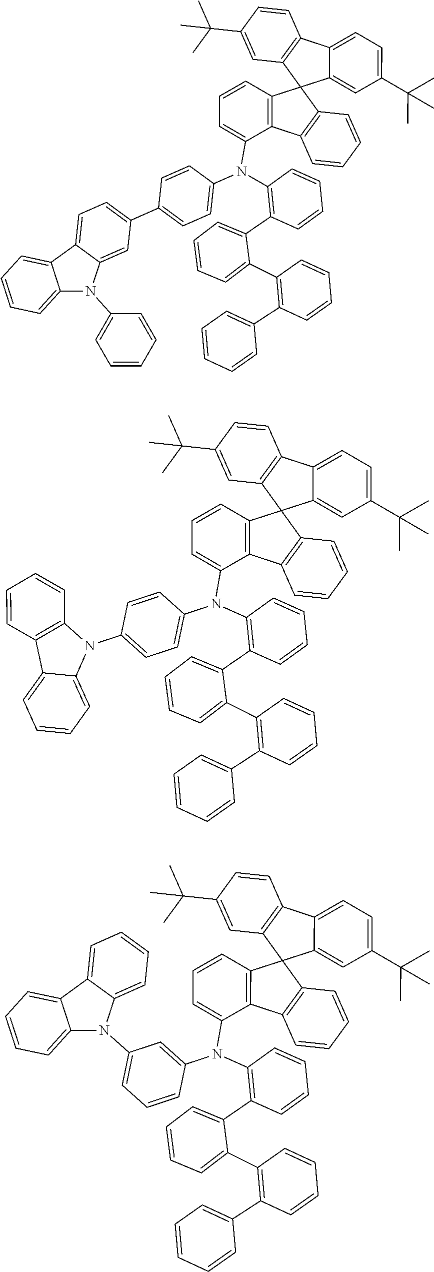 [SMILES: CC(C)(C)c1ccc2c(c1)C1(c3cc(C(C)(C)C)ccc3-2)c2ccccc2-c2c(N(c3ccc(-c4ccc5c6ccccc6n(-c6ccccc6)c5c4)cc3)c3ccccc3-c3ccccc3-c3ccccc3-c3ccccc3)cccc21 ; CC(C)(C)c1ccc2c(c1)C1(c3cc(C(C)(C)C)ccc3-2)c2ccccc2-c2c(N(c3ccc(-n4c5ccccc5c5ccccc54)cc3)c3ccccc3-c3ccccc3-c3ccccc3-c3ccccc3)cccc21 ; CC(C)(C)c1ccc2c(c1)C1(c3cc(C(C)(C)C)ccc3-2)c2ccccc2-c2c(N(c3cccc(-n4c5ccccc5c5ccccc54)c3)c3ccccc3-c3ccccc3-c3ccccc3-c3ccccc3)cccc21]

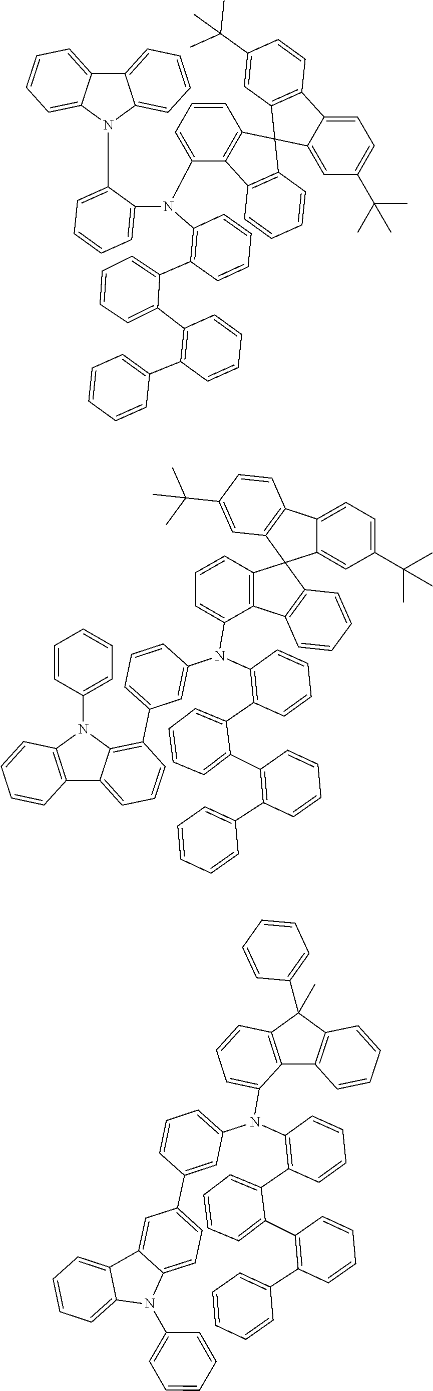 [SMILES: CC(C)(C)c1ccc2c(c1)C1(c3cc(C(C)(C)C)ccc3-2)c2ccccc2-c2c(N(c3ccccc3-c3ccccc3-c3ccccc3-c3ccccc3)c3ccccc3-n3c4ccccc4c4ccccc43)cccc21 ; CC(C)(C)c1ccc2c(c1)C1(c3cc(C(C)(C)C)ccc3-2)c2ccccc2-c2c(N(c3cccc(-c4cccc5c6ccccc6n(-c6ccccc6)c45)c3)c3ccccc3-c3ccccc3-c3ccccc3-c3ccccc3)cccc21 ; CC1(c2ccccc2)c2ccccc2-c2c(N(c3cccc(-c4ccc5c(c4)c4ccccc4n5-c4ccccc4)c3)c3ccccc3-c3ccccc3-c3ccccc3-c3ccccc3)cccc21]

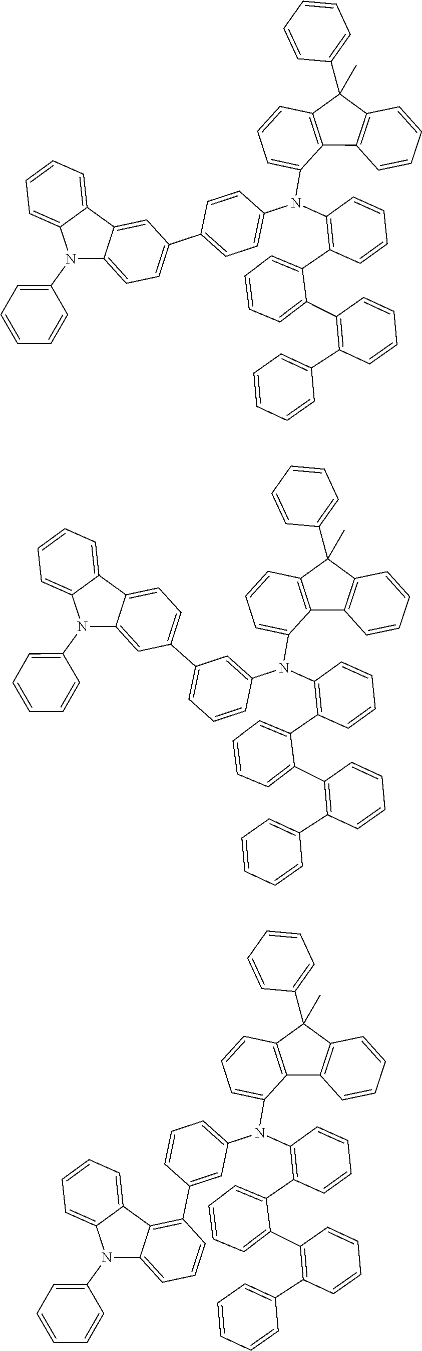

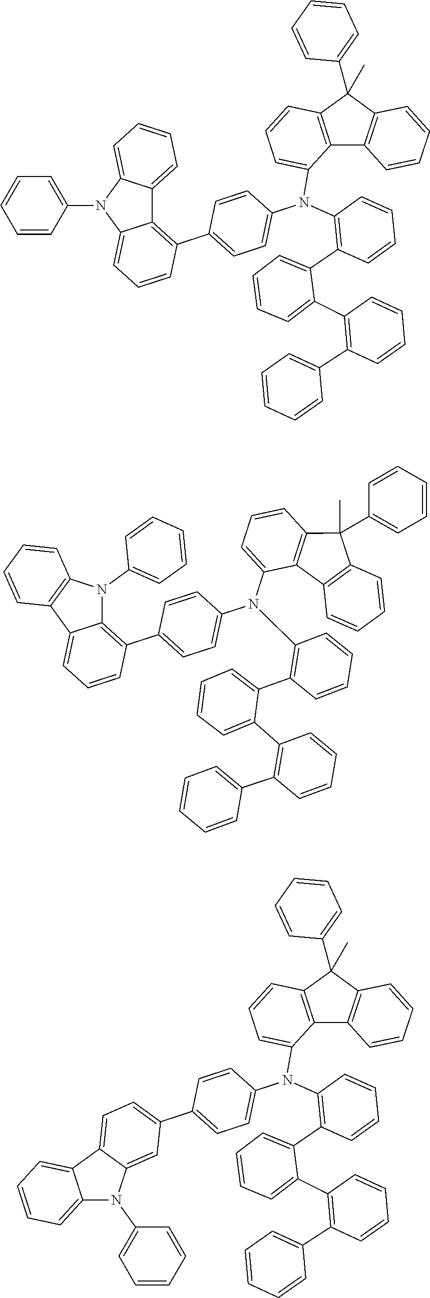 [SMILES: CC1(c2ccccc2)c2ccccc2-c2c(N(c3ccc(-c4cccc5c4c4ccccc4n5-c4ccccc4)cc3)c3ccccc3-c3ccccc3-c3ccccc3-c3ccccc3)cccc21 ; CC1(c2ccccc2)c2ccccc2-c2c(N(c3ccc(-c4cccc5c6ccccc6n(-c6ccccc6)c45)cc3)c3ccccc3-c3ccccc3-c3ccccc3-c3ccccc3)cccc21 ; CC1(c2ccccc2)c2ccccc2-c2c(N(c3ccc(-c4ccc5c6ccccc6n(-c6ccccc6)c5c4)cc3)c3ccccc3-c3ccccc3-c3ccccc3-c3ccccc3)cccc21]

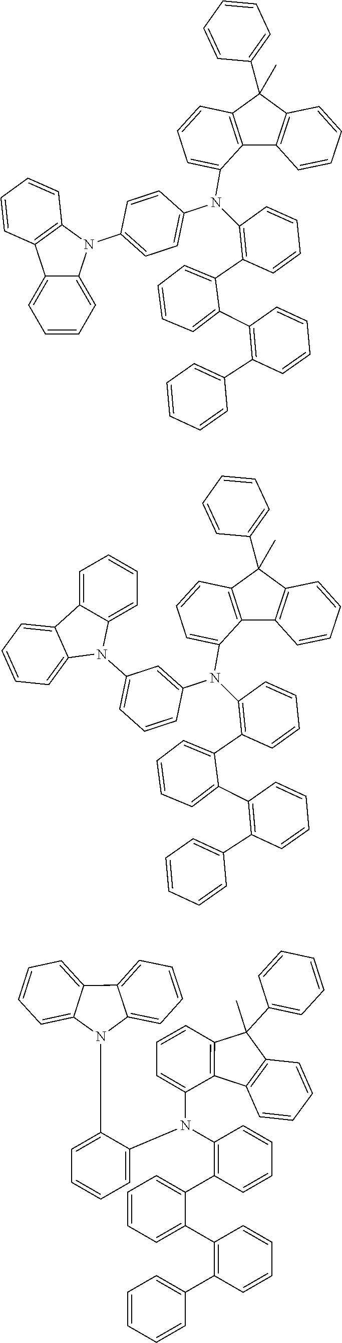 [SMILES: CC1(c2ccccc2)c2ccccc2-c2c(N(c3ccc(-n4c5ccccc5c5ccccc54)cc3)c3ccccc3-c3ccccc3-c3ccccc3-c3ccccc3)cccc21 ; CC1(c2ccccc2)c2ccccc2-c2c(N(c3cccc(-n4c5ccccc5c5ccccc54)c3)c3ccccc3-c3ccccc3-c3ccccc3-c3ccccc3)cccc21 ; CC1(c2ccccc2)c2ccccc2-c2c(N(c3ccccc3-c3ccccc3-c3ccccc3-c3ccccc3)c3ccccc3-n3c4ccccc4c4ccccc43)cccc21]

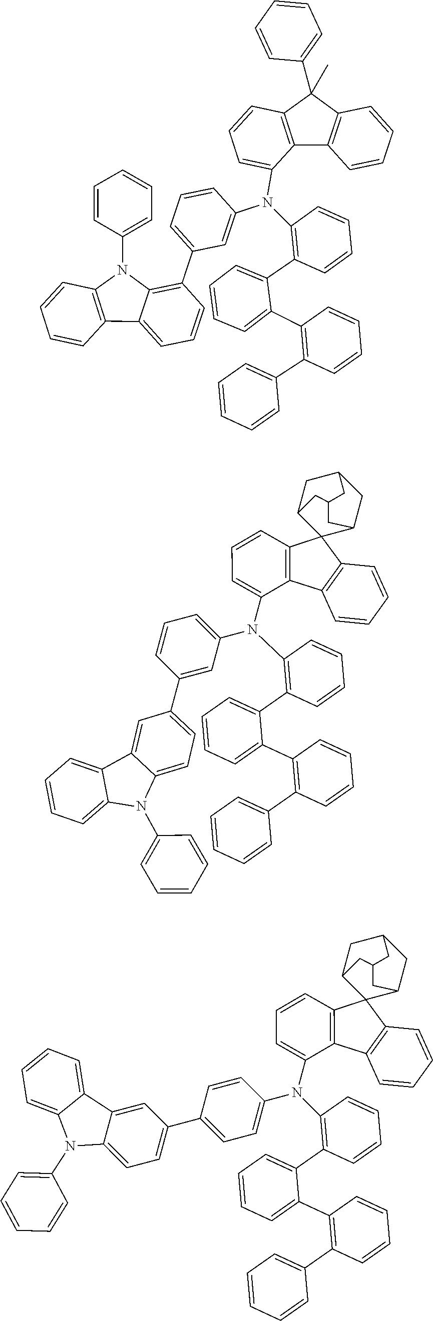 [SMILES: CC1(c2ccccc2)c2ccccc2-c2c(N(c3cccc(-c4cccc5c6ccccc6n(-c6ccccc6)c45)c3)c3ccccc3-c3ccccc3-c3ccccc3-c3ccccc3)cccc21 ; c1ccc(-c2ccccc2-c2ccccc2-c2ccccc2N(c2cccc(-c3ccc4c(c3)c3ccccc3n4-c3ccccc3)c2)c2cccc3c2-c2ccccc2C32C3CC4CC(C3)CC2C4)cc1 ; c1ccc(-c2ccccc2-c2ccccc2-c2ccccc2N(c2ccc(-c3ccc4c(c3)c3ccccc3n4-c3ccccc3)cc2)c2cccc3c2-c2ccccc2C32C3CC4CC(C3)CC2C4)cc1]

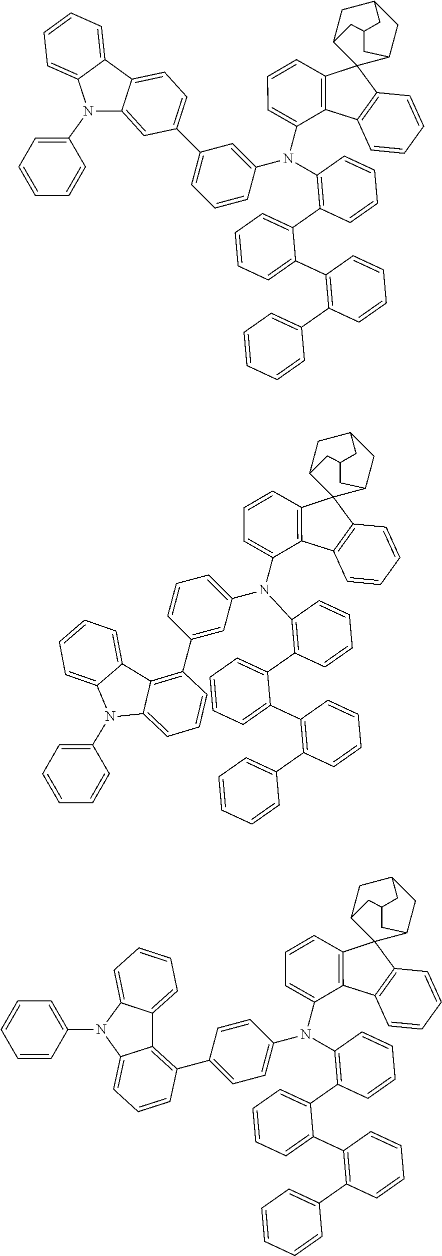 [SMILES: c1ccc(-c2ccccc2-c2ccccc2-c2ccccc2N(c2cccc(-c3ccc4c5ccccc5n(-c5ccccc5)c4c3)c2)c2cccc3c2-c2ccccc2C32C3CC4CC(C3)CC2C4)cc1 ; c1ccc(-c2ccccc2-c2ccccc2-c2ccccc2N(c2cccc(-c3cccc4c3c3ccccc3n4-c3ccccc3)c2)c2cccc3c2-c2ccccc2C32C3CC4CC(C3)CC2C4)cc1 ; c1ccc(-c2ccccc2-c2ccccc2-c2ccccc2N(c2ccc(-c3cccc4c3c3ccccc3n4-c3ccccc3)cc2)c2cccc3c2-c2ccccc2C32C3CC4CC(C3)CC2C4)cc1]

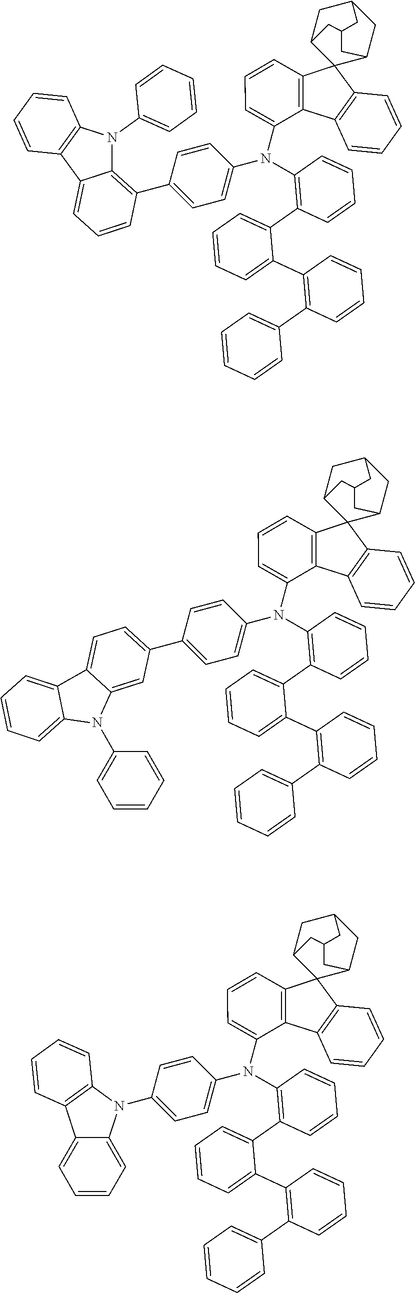 [SMILES: c1ccc(-c2ccccc2-c2ccccc2-c2ccccc2N(c2ccc(-c3cccc4c5ccccc5n(-c5ccccc5)c34)cc2)c2cccc3c2-c2ccccc2C32C3CC4CC(C3)CC2C4)cc1 ; c1ccc(-c2ccccc2-c2ccccc2-c2ccccc2N(c2ccc(-c3ccc4c5ccccc5n(-c5ccccc5)c4c3)cc2)c2cccc3c2-c2ccccc2C32C3CC4CC(C3)CC2C4)cc1 ; c1ccc(-c2ccccc2-c2ccccc2-c2ccccc2N(c2ccc(-n3c4ccccc4c4ccccc43)cc2)c2cccc3c2-c2ccccc2C32C3CC4CC(C3)CC2C4)cc1]

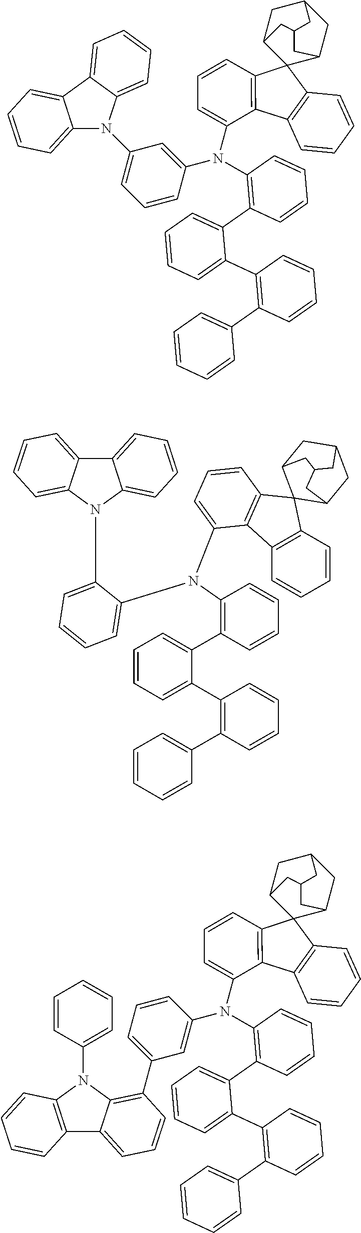 [SMILES: c1ccc(-c2ccccc2-c2ccccc2-c2ccccc2N(c2cccc(-n3c4ccccc4c4ccccc43)c2)c2cccc3c2-c2ccccc2C32C3CC4CC(C3)CC2C4)cc1 ; c1ccc(-c2ccccc2-c2ccccc2-c2ccccc2N(c2ccccc2-n2c3ccccc3c3ccccc32)c2cccc3c2-c2ccccc2C32C3CC4CC(C3)CC2C4)cc1 ; c1ccc(-c2ccccc2-c2ccccc2-c2ccccc2N(c2cccc(-c3cccc4c5ccccc5n(-c5ccccc5)c34)c2)c2cccc3c2-c2ccccc2C32C3CC4CC(C3)CC2C4)cc1]

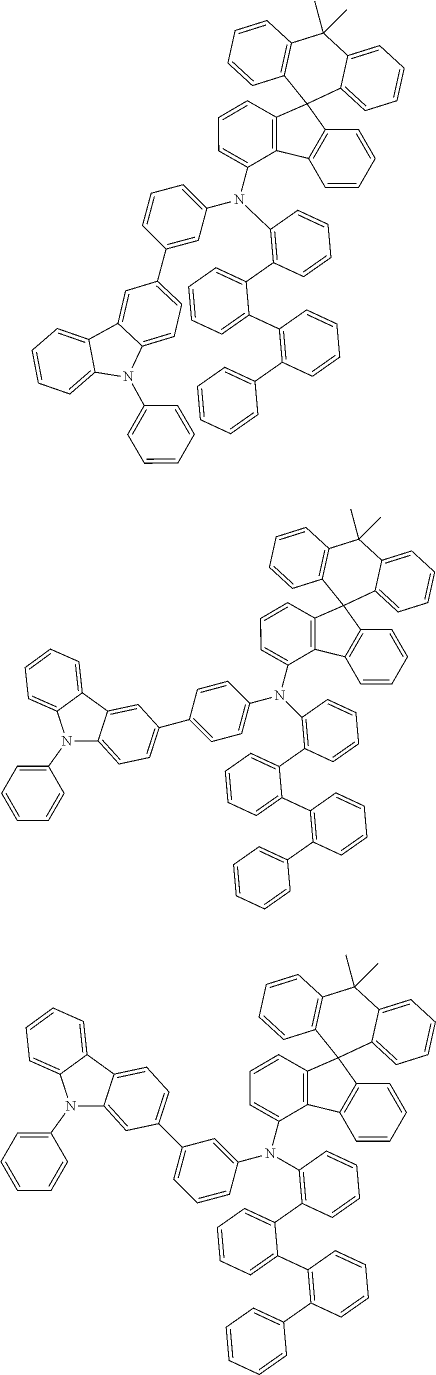 [SMILES: CC1(C)c2ccccc2C2(c3ccccc3-c3c(N(c4cccc(-c5ccc6c(c5)c5ccccc5n6-c5ccccc5)c4)c4ccccc4-c4ccccc4-c4ccccc4-c4ccccc4)cccc32)c2ccccc21 ; CC1(C)c2ccccc2C2(c3ccccc3-c3c(N(c4ccc(-c5ccc6c(c5)c5ccccc5n6-c5ccccc5)cc4)c4ccccc4-c4ccccc4-c4ccccc4-c4ccccc4)cccc32)c2ccccc21 ; CC1(C)c2ccccc2C2(c3ccccc3-c3c(N(c4cccc(-c5ccc6c7ccccc7n(-c7ccccc7)c6c5)c4)c4ccccc4-c4ccccc4-c4ccccc4-c4ccccc4)cccc32)c2ccccc21]

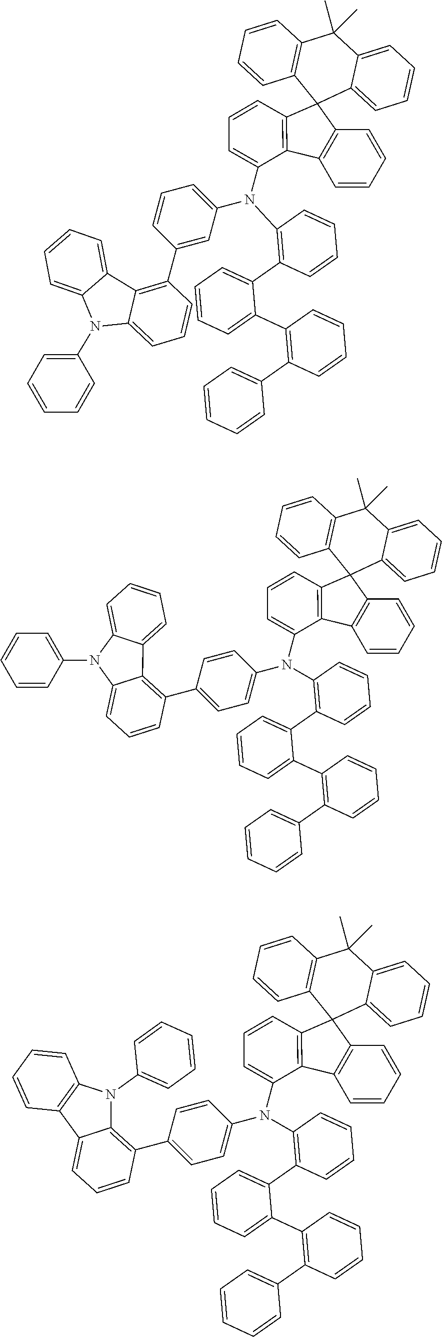 [SMILES: CC1(C)c2ccccc2C2(c3ccccc3-c3c(N(c4cccc(-c5cccc6c5c5ccccc5n6-c5ccccc5)c4)c4ccccc4-c4ccccc4-c4ccccc4-c4ccccc4)cccc32)c2ccccc21 ; CC1(C)c2ccccc2C2(c3ccccc3-c3c(N(c4ccc(-c5cccc6c5c5ccccc5n6-c5ccccc5)cc4)c4ccccc4-c4ccccc4-c4ccccc4-c4ccccc4)cccc32)c2ccccc21 ; CC1(C)c2ccccc2C2(c3ccccc3-c3c(N(c4ccc(-c5cccc6c7ccccc7n(-c7ccccc7)c56)cc4)c4ccccc4-c4ccccc4-c4ccccc4-c4ccccc4)cccc32)c2ccccc21]

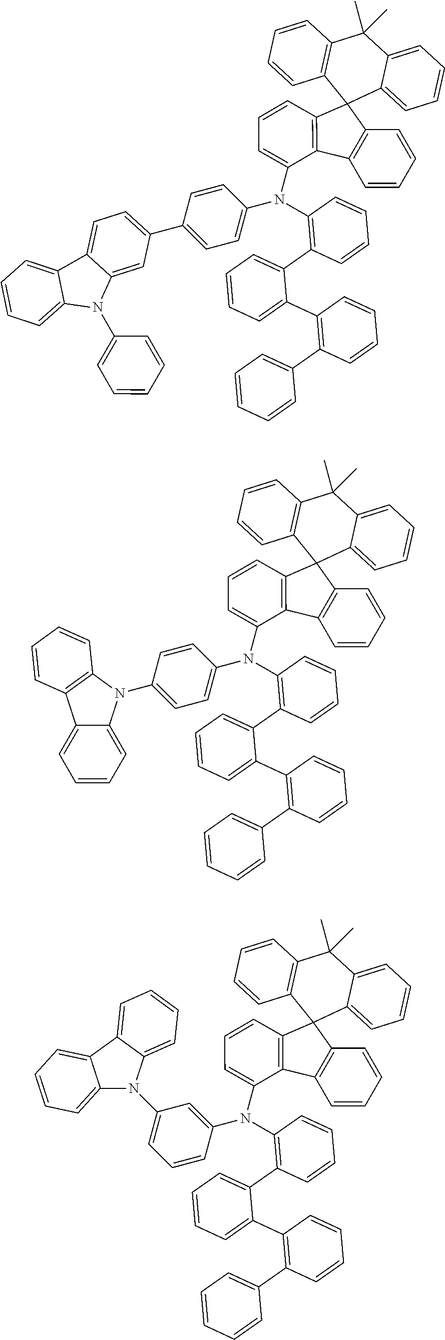 [SMILES: CC1(C)c2ccccc2C2(c3ccccc3-c3c(N(c4ccc(-c5ccc6c7ccccc7n(-c7ccccc7)c6c5)cc4)c4ccccc4-c4ccccc4-c4ccccc4-c4ccccc4)cccc32)c2ccccc21 ; CC1(C)c2ccccc2C2(c3ccccc3-c3c(N(c4ccc(-n5c6ccccc6c6ccccc65)cc4)c4ccccc4-c4ccccc4-c4ccccc4-c4ccccc4)cccc32)c2ccccc21 ; CC1(C)c2ccccc2C2(c3ccccc3-c3c(N(c4cccc(-n5c6ccccc6c6ccccc65)c4)c4ccccc4-c4ccccc4-c4ccccc4-c4ccccc4)cccc32)c2ccccc21]

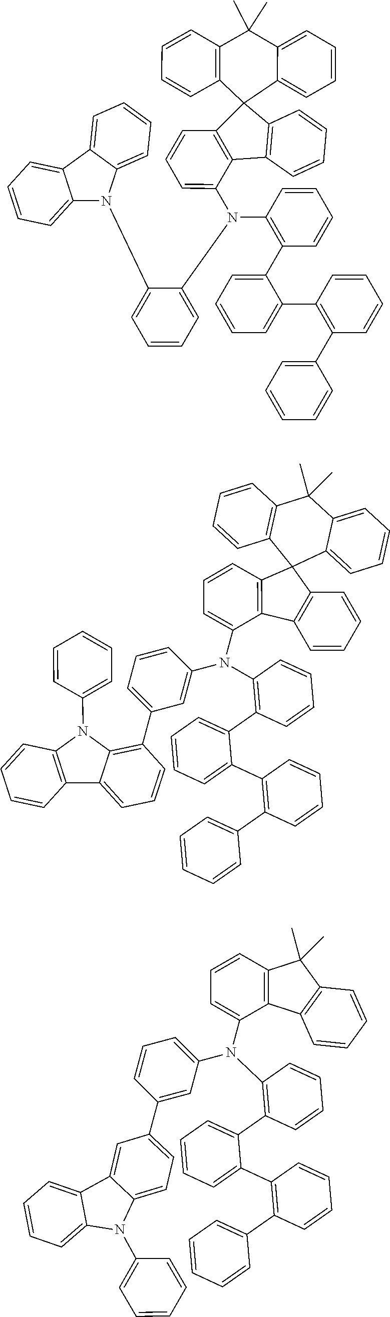 [SMILES: CC1(C)c2ccccc2C2(c3ccccc3-c3c(N(c4ccccc4-c4ccccc4-c4ccccc4-c4ccccc4)c4ccccc4-n4c5ccccc5c5ccccc54)cccc32)c2ccccc21 ; CC1(C)c2ccccc2C2(c3ccccc3-c3c(N(c4cccc(-c5cccc6c7ccccc7n(-c7ccccc7)c56)c4)c4ccccc4-c4ccccc4-c4ccccc4-c4ccccc4)cccc32)c2ccccc21 ; CC1(C)c2ccccc2-c2c(N(c3cccc(-c4ccc5c(c4)c4ccccc4n5-c4ccccc4)c3)c3ccccc3-c3ccccc3-c3ccccc3-c3ccccc3)cccc21]

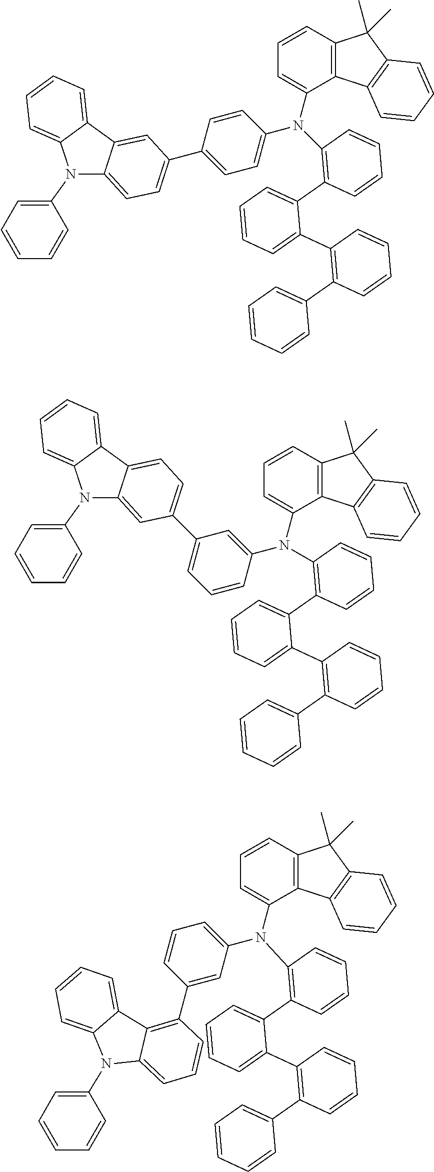 [SMILES: CC1(C)c2ccccc2-c2c(N(c3ccc(-c4ccc5c(c4)c4ccccc4n5-c4ccccc4)cc3)c3ccccc3-c3ccccc3-c3ccccc3-c3ccccc3)cccc21 ; CC1(C)c2ccccc2-c2c(N(c3cccc(-c4ccc5c6ccccc6n(-c6ccccc6)c5c4)c3)c3ccccc3-c3ccccc3-c3ccccc3-c3ccccc3)cccc21 ; CC1(C)c2ccccc2-c2c(N(c3cccc(-c4cccc5c4c4ccccc4n5-c4ccccc4)c3)c3ccccc3-c3ccccc3-c3ccccc3-c3ccccc3)cccc21]

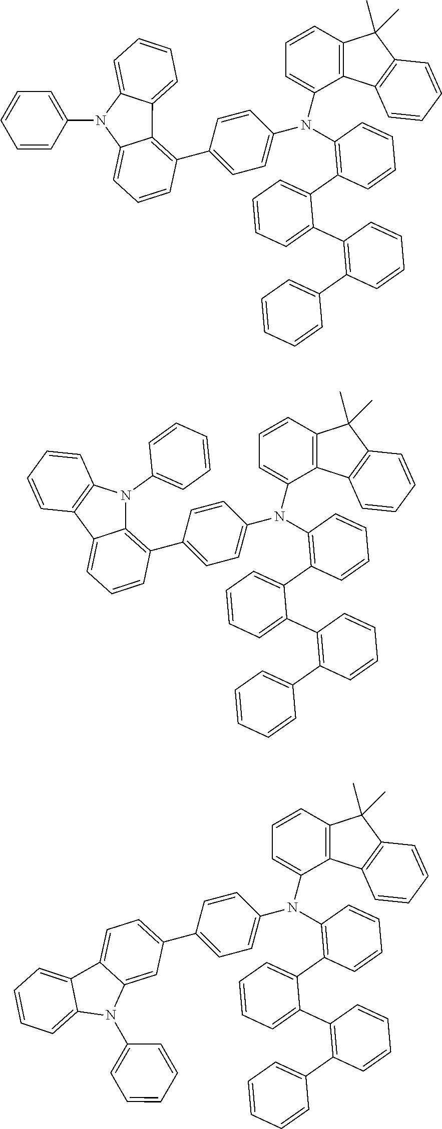 [SMILES: CC1(C)c2ccccc2-c2c(N(c3ccc(-c4cccc5c4c4ccccc4n5-c4ccccc4)cc3)c3ccccc3-c3ccccc3-c3ccccc3-c3ccccc3)cccc21 ; CC1(C)c2ccccc2-c2c(N(c3ccc(-c4cccc5c6ccccc6n(-c6ccccc6)c45)cc3)c3ccccc3-c3ccccc3-c3ccccc3-c3ccccc3)cccc21 ; CC1(C)c2ccccc2-c2c(N(c3ccc(-c4ccc5c6ccccc6n(-c6ccccc6)c5c4)cc3)c3ccccc3-c3ccccc3-c3ccccc3-c3ccccc3)cccc21]

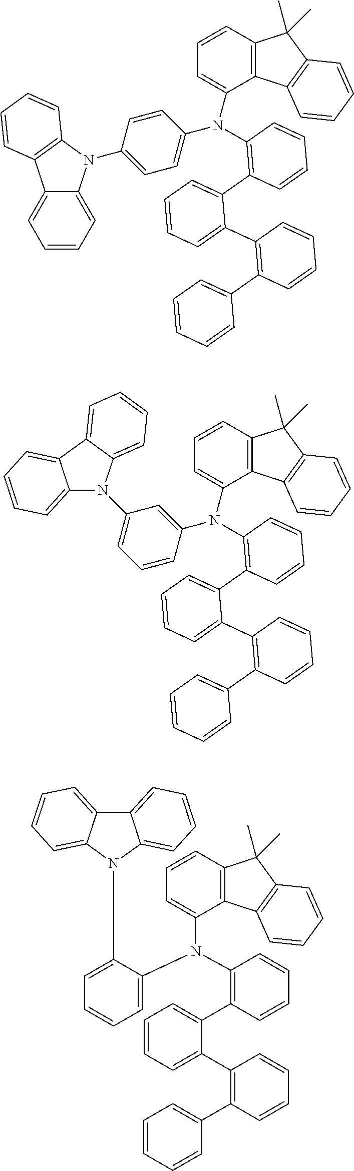 [SMILES: CC1(C)c2ccccc2-c2c(N(c3ccc(-n4c5ccccc5c5ccccc54)cc3)c3ccccc3-c3ccccc3-c3ccccc3-c3ccccc3)cccc21 ; CC1(C)c2ccccc2-c2c(N(c3cccc(-n4c5ccccc5c5ccccc54)c3)c3ccccc3-c3ccccc3-c3ccccc3-c3ccccc3)cccc21 ; CC1(C)c2ccccc2-c2c(N(c3ccccc3-c3ccccc3-c3ccccc3-c3ccccc3)c3ccccc3-n3c4ccccc4c4ccccc43)cccc21]

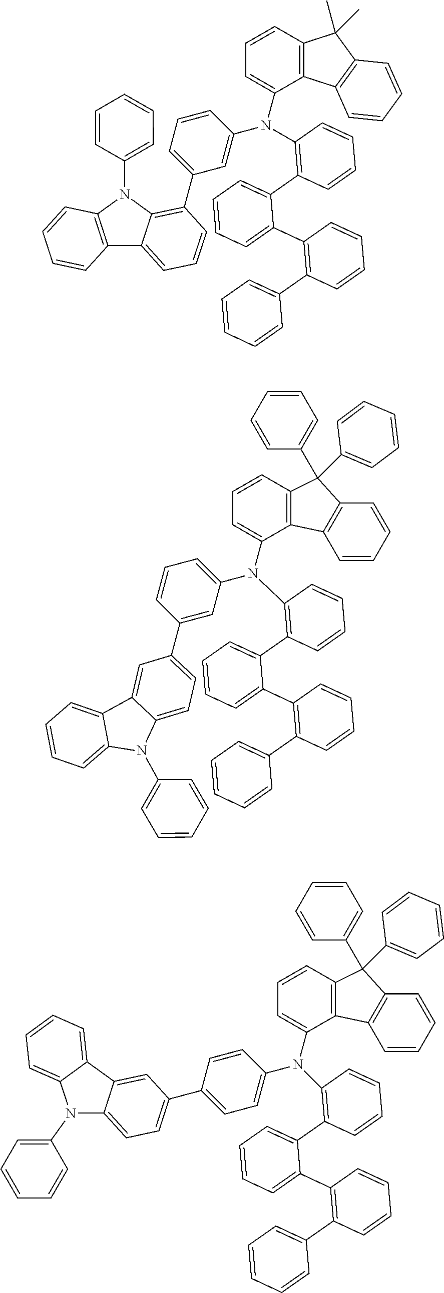 [SMILES: CC1(C)c2ccccc2-c2c(N(c3cccc(-c4cccc5c6ccccc6n(-c6ccccc6)c45)c3)c3ccccc3-c3ccccc3-c3ccccc3-c3ccccc3)cccc21 ; c1ccc(-c2ccccc2-c2ccccc2-c2ccccc2N(c2cccc(-c3ccc4c(c3)c3ccccc3n4-c3ccccc3)c2)c2cccc3c2-c2ccccc2C3(c2ccccc2)c2ccccc2)cc1 ; c1ccc(-c2ccccc2-c2ccccc2-c2ccccc2N(c2ccc(-c3ccc4c(c3)c3ccccc3n4-c3ccccc3)cc2)c2cccc3c2-c2ccccc2C3(c2ccccc2)c2ccccc2)cc1]

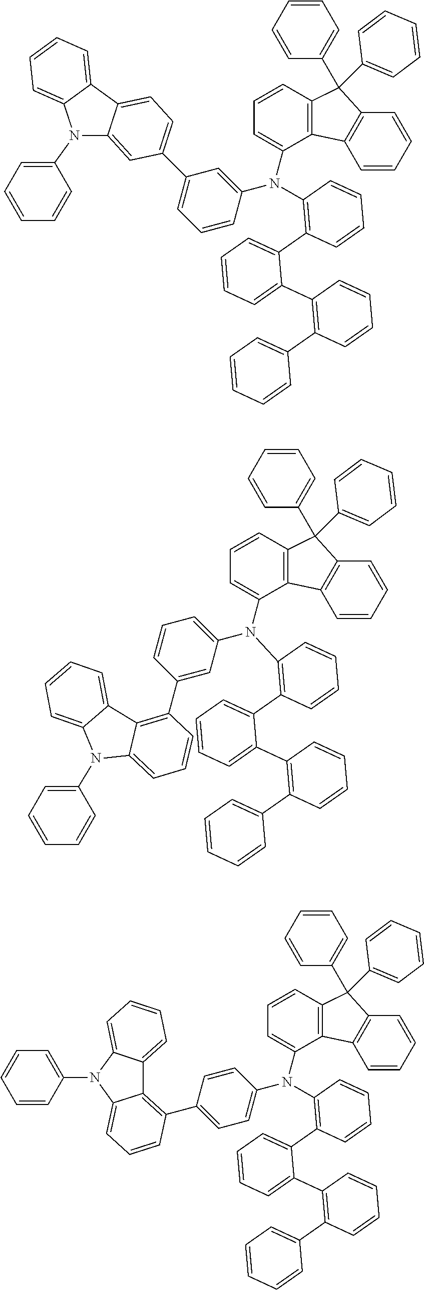

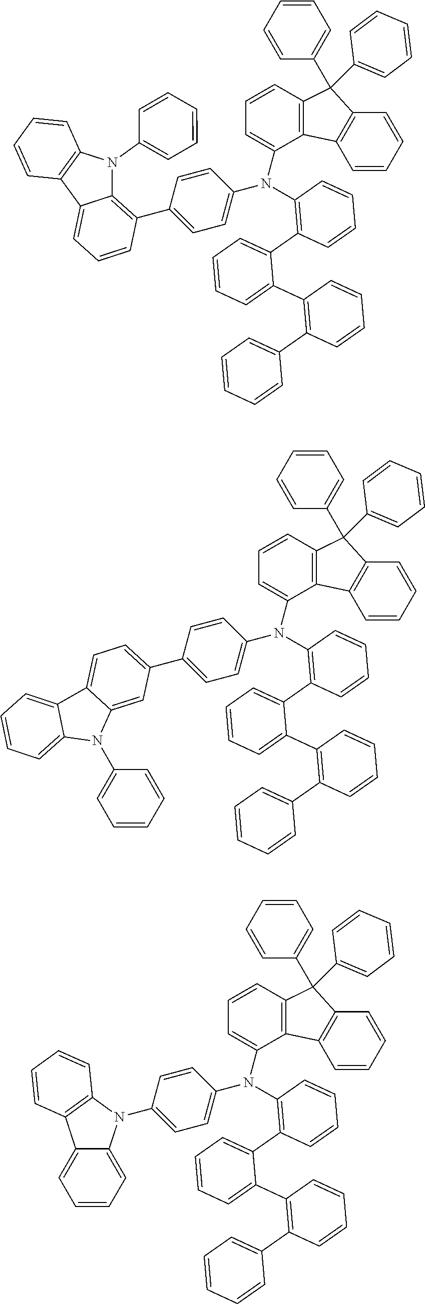 [SMILES: c1ccc(-c2ccccc2-c2ccccc2-c2ccccc2N(c2ccc(-c3cccc4c5ccccc5n(-c5ccccc5)c34)cc2)c2cccc3c2-c2ccccc2C3(c2ccccc2)c2ccccc2)cc1 ; c1ccc(-c2ccccc2-c2ccccc2-c2ccccc2N(c2ccc(-c3ccc4c5ccccc5n(-c5ccccc5)c4c3)cc2)c2cccc3c2-c2ccccc2C3(c2ccccc2)c2ccccc2)cc1 ; c1ccc(-c2ccccc2-c2ccccc2-c2ccccc2N(c2ccc(-n3c4ccccc4c4ccccc43)cc2)c2cccc3c2-c2ccccc2C3(c2ccccc2)c2ccccc2)cc1]

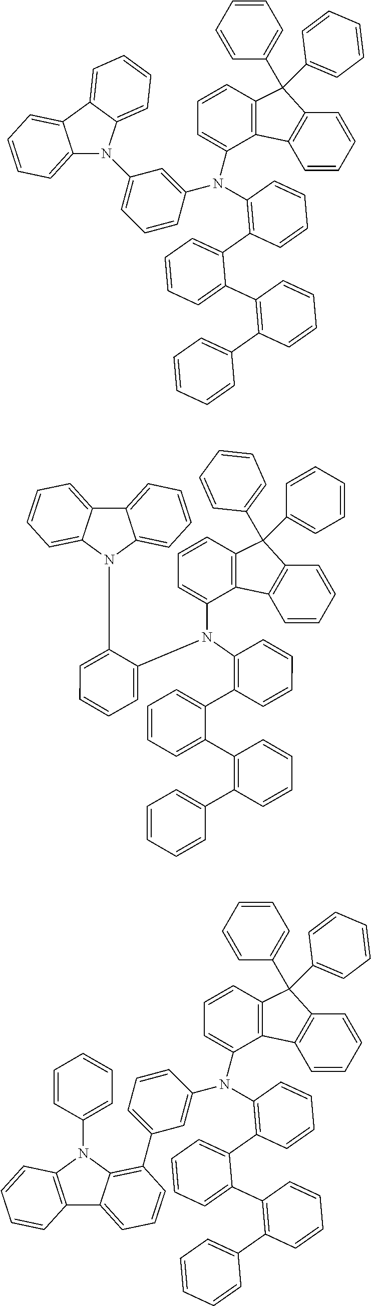 [SMILES: c1ccc(-c2ccccc2-c2ccccc2-c2ccccc2N(c2cccc(-n3c4ccccc4c4ccccc43)c2)c2cccc3c2-c2ccccc2C3(c2ccccc2)c2ccccc2)cc1 ; c1ccc(-c2ccccc2-c2ccccc2-c2ccccc2N(c2ccccc2-n2c3ccccc3c3ccccc32)c2cccc3c2-c2ccccc2C3(c2ccccc2)c2ccccc2)cc1 ; c1ccc(-c2ccccc2-c2ccccc2-c2ccccc2N(c2cccc(-c3cccc4c5ccccc5n(-c5ccccc5)c34)c2)c2cccc3c2-c2ccccc2C3(c2ccccc2)c2ccccc2)cc1]

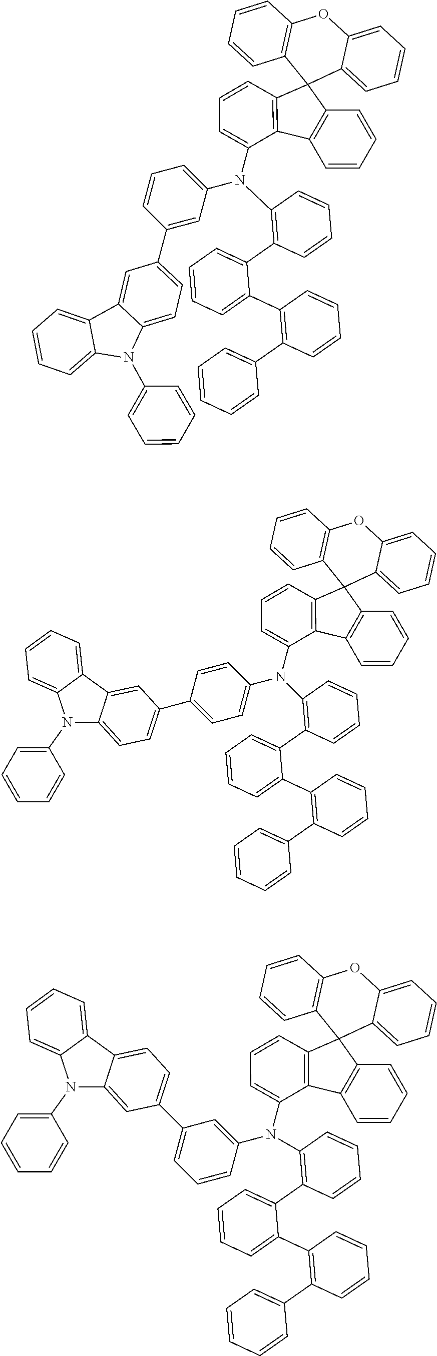 [SMILES: c1ccc(-c2ccccc2-c2ccccc2-c2ccccc2N(c2cccc(-c3ccc4c(c3)c3ccccc3n4-c3ccccc3)c2)c2cccc3c2-c2ccccc2C32c3ccccc3Oc3ccccc32)cc1 ; c1ccc(-c2ccccc2-c2ccccc2-c2ccccc2N(c2ccc(-c3ccc4c(c3)c3ccccc3n4-c3ccccc3)cc2)c2cccc3c2-c2ccccc2C32c3ccccc3Oc3ccccc32)cc1 ; c1ccc(-c2ccccc2-c2ccccc2-c2ccccc2N(c2cccc(-c3ccc4c5ccccc5n(-c5ccccc5)c4c3)c2)c2cccc3c2-c2ccccc2C32c3ccccc3Oc3ccccc32)cc1]

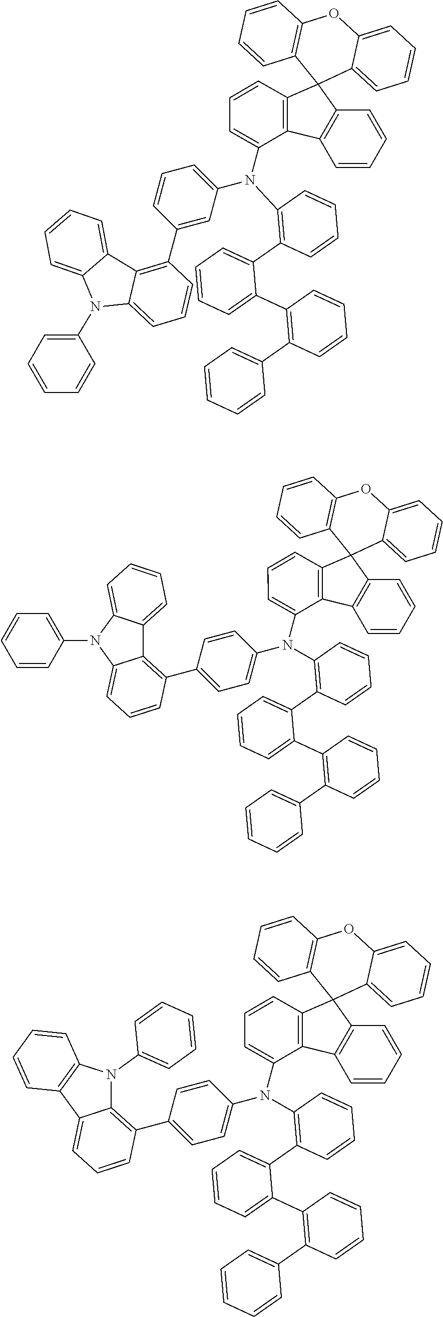 [SMILES: c1ccc(-c2ccccc2-c2ccccc2-c2ccccc2N(c2cccc(-c3cccc4c3c3ccccc3n4-c3ccccc3)c2)c2cccc3c2-c2ccccc2C32c3ccccc3Oc3ccccc32)cc1 ; c1ccc(-c2ccccc2-c2ccccc2-c2ccccc2N(c2ccc(-c3cccc4c3c3ccccc3n4-c3ccccc3)cc2)c2cccc3c2-c2ccccc2C32c3ccccc3Oc3ccccc32)cc1 ; c1ccc(-c2ccccc2-c2ccccc2-c2ccccc2N(c2ccc(-c3cccc4c5ccccc5n(-c5ccccc5)c34)cc2)c2cccc3c2-c2ccccc2C32c3ccccc3Oc3ccccc32)cc1]

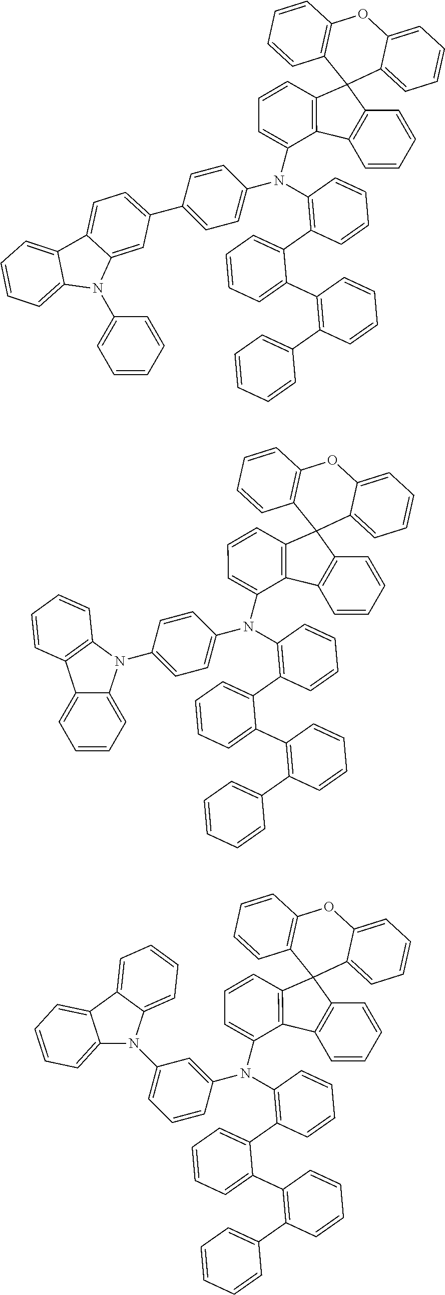 [SMILES: c1ccc(-c2ccccc2-c2ccccc2-c2ccccc2N(c2ccc(-c3ccc4c5ccccc5n(-c5ccccc5)c4c3)cc2)c2cccc3c2-c2ccccc2C32c3ccccc3Oc3ccccc32)cc1 ; c1ccc(-c2ccccc2-c2ccccc2-c2ccccc2N(c2ccc(-n3c4ccccc4c4ccccc43)cc2)c2cccc3c2-c2ccccc2C32c3ccccc3Oc3ccccc32)cc1 ; c1ccc(-c2ccccc2-c2ccccc2-c2ccccc2N(c2cccc(-n3c4ccccc4c4ccccc43)c2)c2cccc3c2-c2ccccc2C32c3ccccc3Oc3ccccc32)cc1]

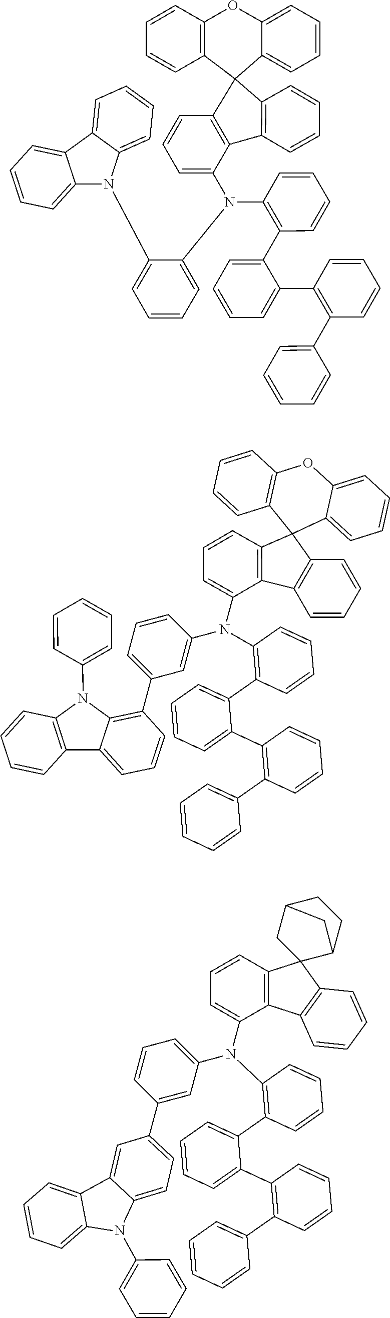 [SMILES: c1ccc(-c2ccccc2-c2ccccc2-c2ccccc2N(c2ccccc2-n2c3ccccc3c3ccccc32)c2cccc3c2-c2ccccc2C32c3ccccc3Oc3ccccc32)cc1 ; c1ccc(-c2ccccc2-c2ccccc2-c2ccccc2N(c2cccc(-c3cccc4c5ccccc5n(-c5ccccc5)c34)c2)c2cccc3c2-c2ccccc2C32c3ccccc3Oc3ccccc32)cc1 ; c1ccc(-c2ccccc2-c2ccccc2-c2ccccc2N(c2cccc(-c3ccc4c(c3)c3ccccc3n4-c3ccccc3)c2)c2cccc3c2-c2ccccc2C32CC3CCC2C3)cc1]

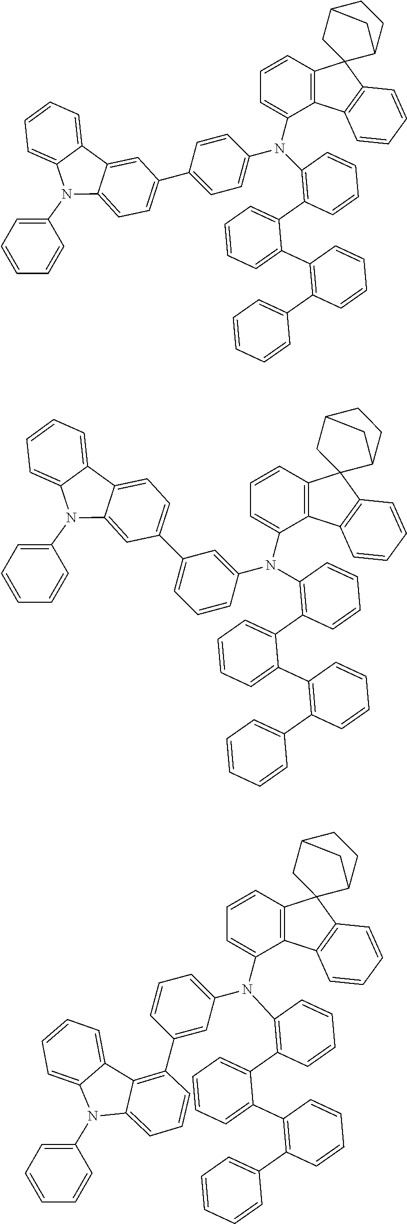 [SMILES: c1ccc(-c2ccccc2-c2ccccc2-c2ccccc2N(c2ccc(-c3ccc4c(c3)c3ccccc3n4-c3ccccc3)cc2)c2cccc3c2-c2ccccc2C32CC3CCC2C3)cc1 ; c1ccc(-c2ccccc2-c2ccccc2-c2ccccc2N(c2cccc(-c3ccc4c5ccccc5n(-c5ccccc5)c4c3)c2)c2cccc3c2-c2ccccc2C32CC3CCC2C3)cc1 ; c1ccc(-c2ccccc2-c2ccccc2-c2ccccc2N(c2cccc(-c3cccc4c3c3ccccc3n4-c3ccccc3)c2)c2cccc3c2-c2ccccc2C32CC3CCC2C3)cc1]

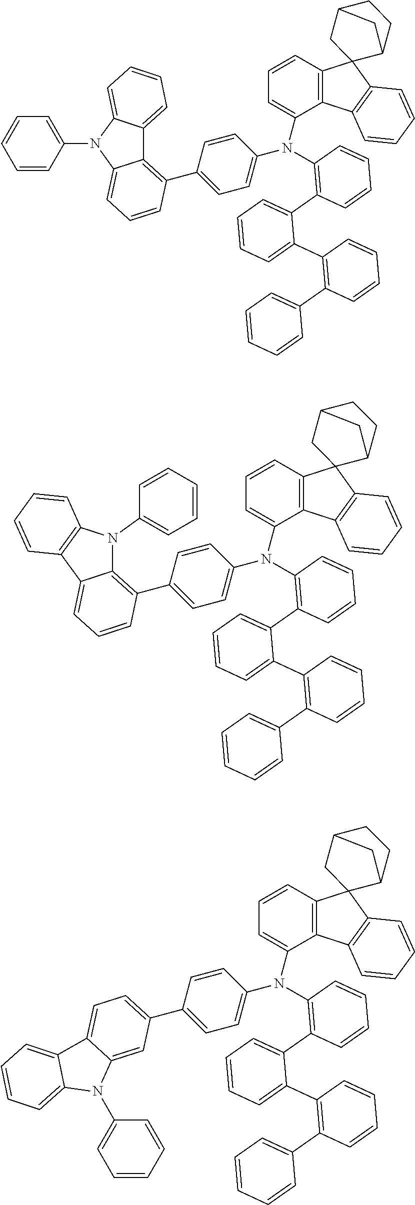 [SMILES: c1ccc(-c2ccccc2-c2ccccc2-c2ccccc2N(c2ccc(-c3cccc4c3c3ccccc3n4-c3ccccc3)cc2)c2cccc3c2-c2ccccc2C32CC3CCC2C3)cc1 ; c1ccc(-c2ccccc2-c2ccccc2-c2ccccc2N(c2ccc(-c3cccc4c5ccccc5n(-c5ccccc5)c34)cc2)c2cccc3c2-c2ccccc2C32CC3CCC2C3)cc1 ; c1ccc(-c2ccccc2-c2ccccc2-c2ccccc2N(c2ccc(-c3ccc4c5ccccc5n(-c5ccccc5)c4c3)cc2)c2cccc3c2-c2ccccc2C32CC3CCC2C3)cc1]

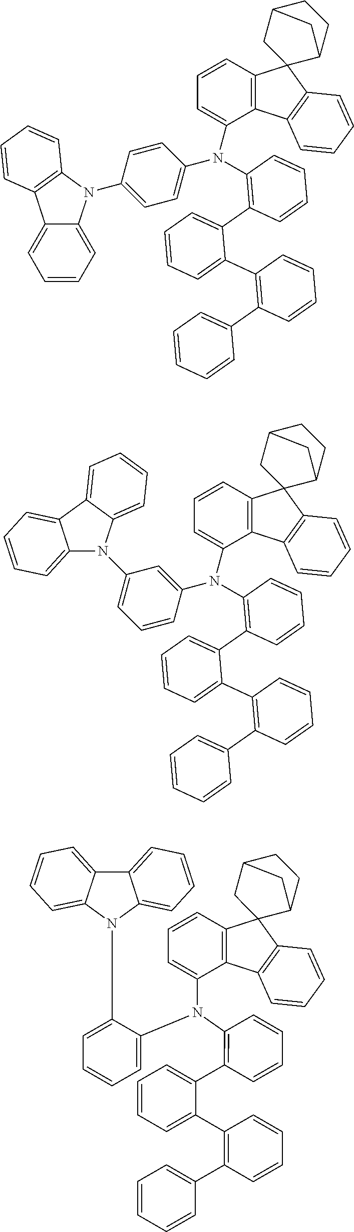 [SMILES: c1ccc(-c2ccccc2-c2ccccc2-c2ccccc2N(c2ccc(-n3c4ccccc4c4ccccc43)cc2)c2cccc3c2-c2ccccc2C32CC3CCC2C3)cc1 ; c1ccc(-c2ccccc2-c2ccccc2-c2ccccc2N(c2cccc(-n3c4ccccc4c4ccccc43)c2)c2cccc3c2-c2ccccc2C32CC3CCC2C3)cc1 ; c1ccc(-c2ccccc2-c2ccccc2-c2ccccc2N(c2ccccc2-n2c3ccccc3c3ccccc32)c2cccc3c2-c2ccccc2C32CC3CCC2C3)cc1]

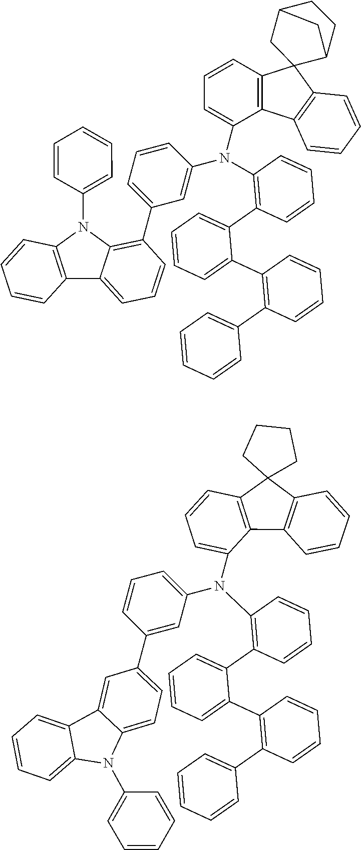 [SMILES: c1ccc(-c2ccccc2-c2ccccc2-c2ccccc2N(c2cccc(-c3cccc4c5ccccc5n(-c5ccccc5)c34)c2)c2cccc3c2-c2ccccc2C32CC3CCC2C3)cc1 ; c1ccc(-c2ccccc2-c2ccccc2-c2ccccc2N(c2cccc(-c3ccc4c(c3)c3ccccc3n4-c3ccccc3)c2)c2cccc3c2-c2ccccc2C32CCCC2)cc1]

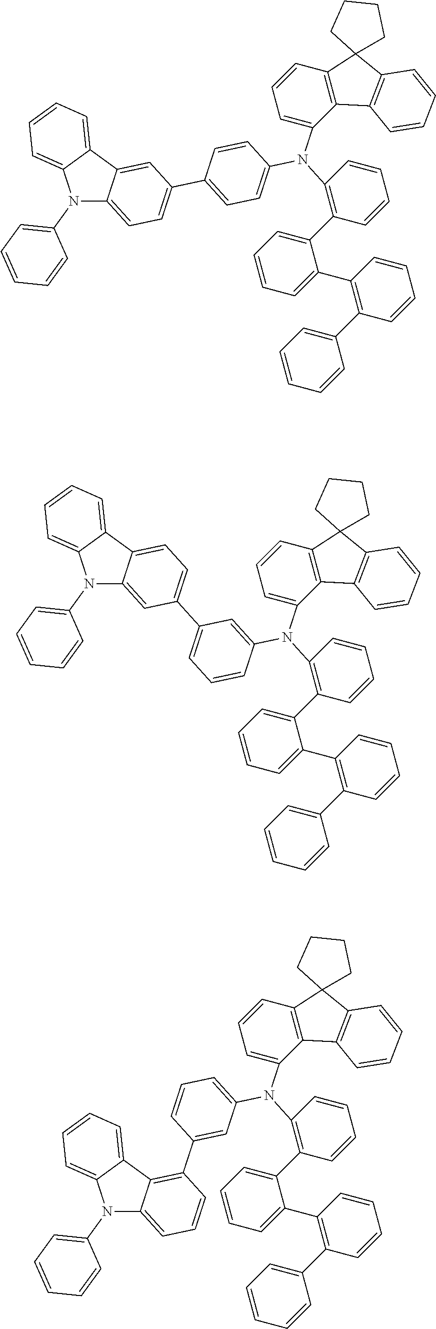 [SMILES: c1ccc(-c2ccccc2-c2ccccc2-c2ccccc2N(c2ccc(-c3ccc4c(c3)c3ccccc3n4-c3ccccc3)cc2)c2cccc3c2-c2ccccc2C32CCCC2)cc1 ; c1ccc(-c2ccccc2-c2ccccc2-c2ccccc2N(c2cccc(-c3ccc4c5ccccc5n(-c5ccccc5)c4c3)c2)c2cccc3c2-c2ccccc2C32CCCC2)cc1 ; c1ccc(-c2ccccc2-c2ccccc2-c2ccccc2N(c2cccc(-c3cccc4c3c3ccccc3n4-c3ccccc3)c2)c2cccc3c2-c2ccccc2C32CCCC2)cc1]

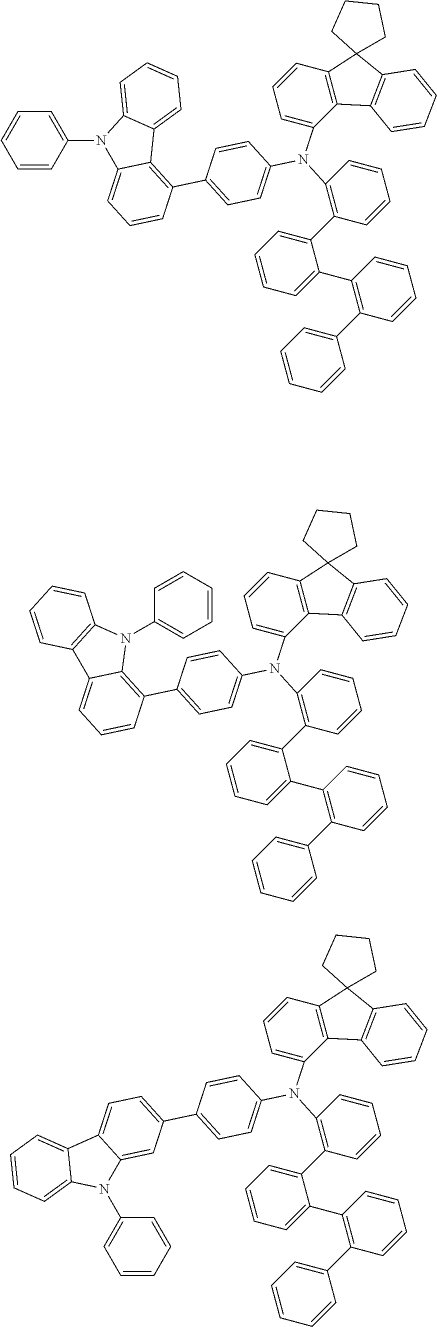 [SMILES: c1ccc(-c2ccccc2-c2ccccc2-c2ccccc2N(c2ccc(-c3cccc4c3c3ccccc3n4-c3ccccc3)cc2)c2cccc3c2-c2ccccc2C32CCCC2)cc1 ; c1ccc(-c2ccccc2-c2ccccc2-c2ccccc2N(c2ccc(-c3cccc4c5ccccc5n(-c5ccccc5)c34)cc2)c2cccc3c2-c2ccccc2C32CCCC2)cc1 ; c1ccc(-c2ccccc2-c2ccccc2-c2ccccc2N(c2ccc(-c3ccc4c5ccccc5n(-c5ccccc5)c4c3)cc2)c2cccc3c2-c2ccccc2C32CCCC2)cc1]

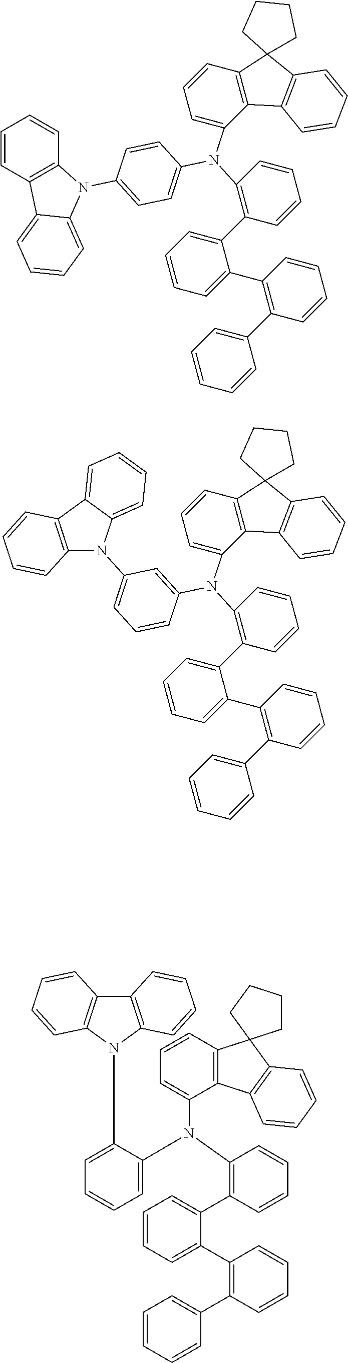

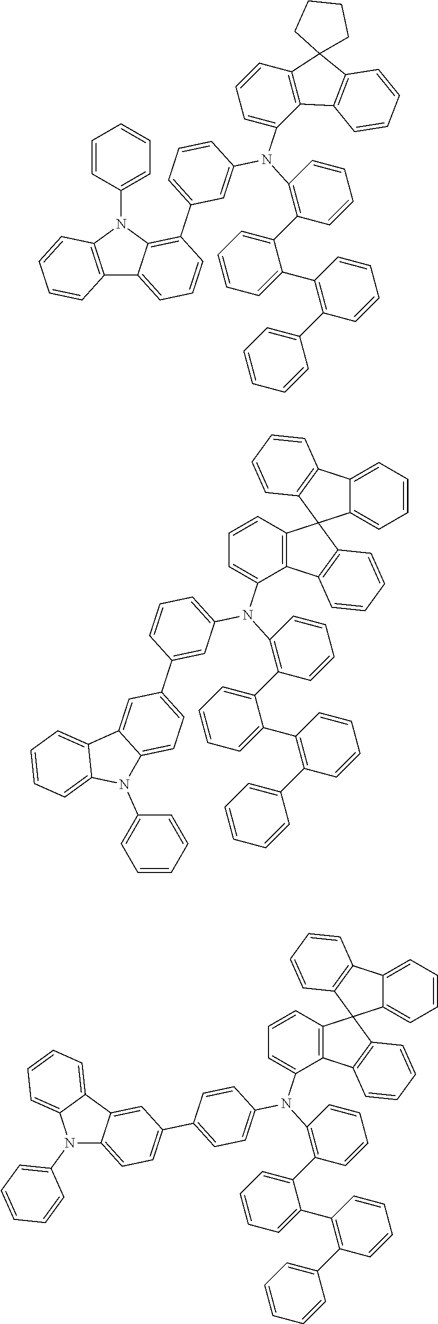

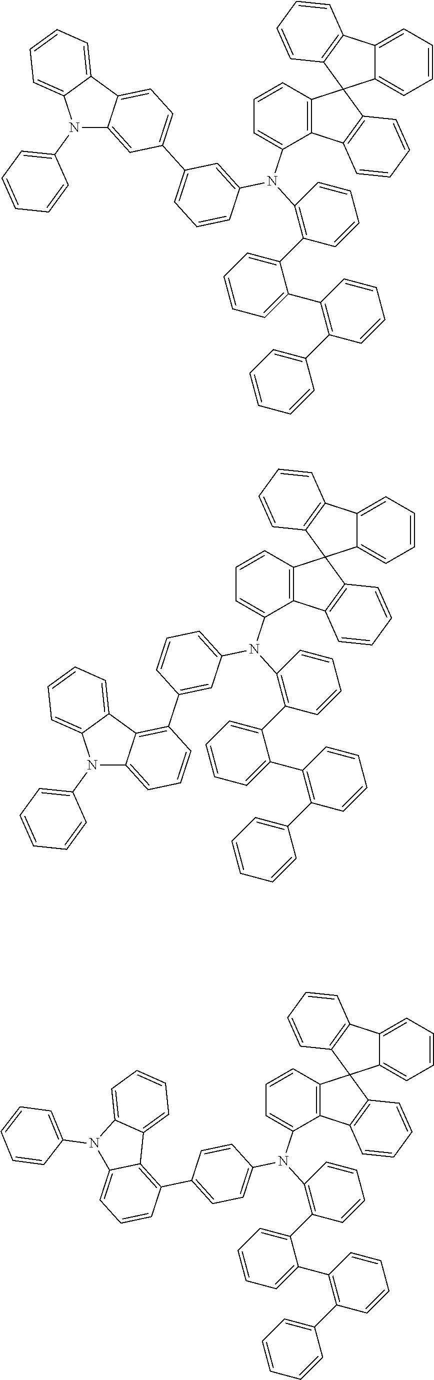 [SMILES: c1ccc(-c2ccccc2-c2ccccc2-c2ccccc2N(c2cccc(-c3ccc4c5ccccc5n(-c5ccccc5)c4c3)c2)c2cccc3c2-c2ccccc2C32c3ccccc3-c3ccccc32)cc1 ; c1ccc(-c2ccccc2-c2ccccc2-c2ccccc2N(c2cccc(-c3cccc4c3c3ccccc3n4-c3ccccc3)c2)c2cccc3c2-c2ccccc2C32c3ccccc3-c3ccccc32)cc1 ; c1ccc(-c2ccccc2-c2ccccc2-c2ccccc2N(c2ccc(-c3cccc4c3c3ccccc3n4-c3ccccc3)cc2)c2cccc3c2-c2ccccc2C32c3ccccc3-c3ccccc32)cc1]

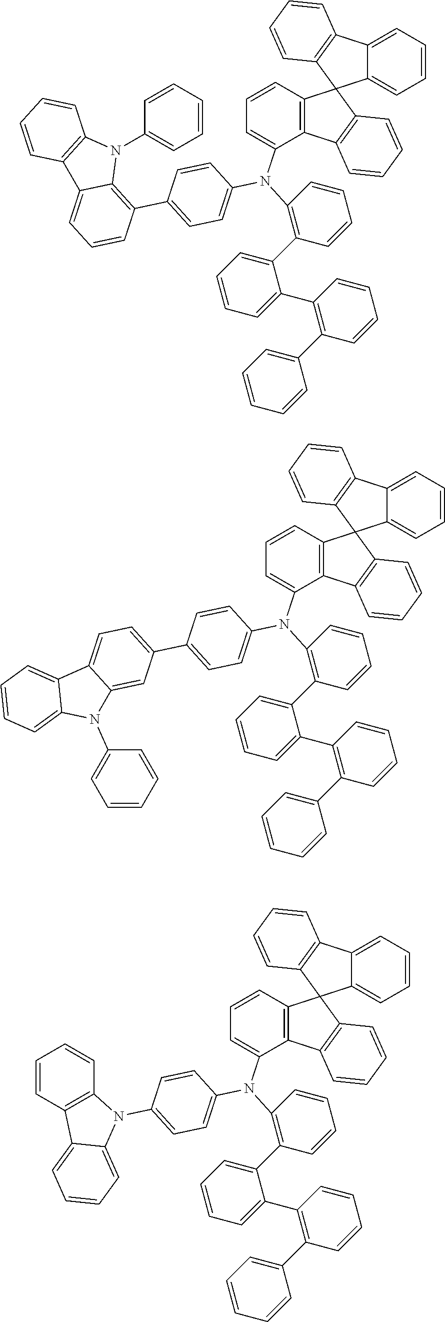

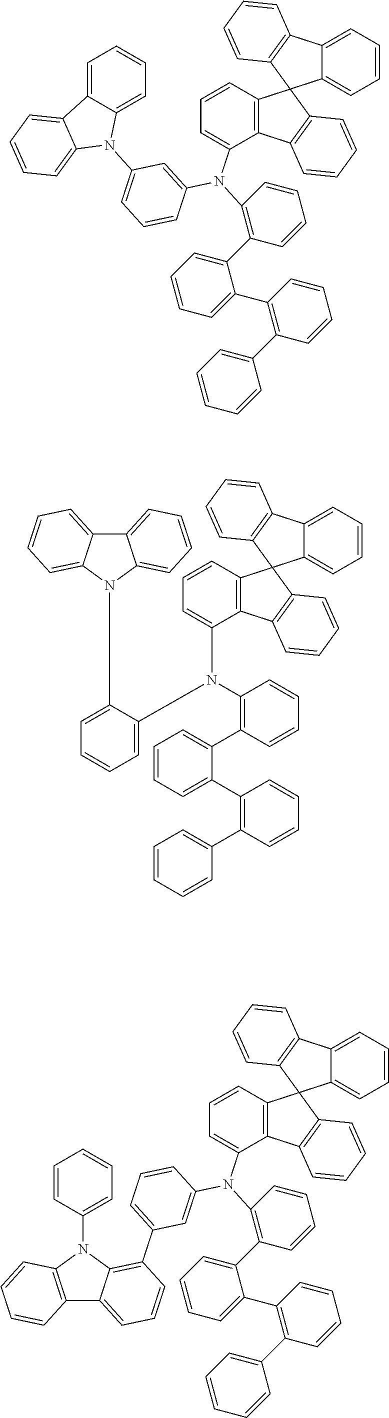 [SMILES: c1ccc(-c2ccccc2-c2ccccc2-c2ccccc2N(c2cccc(-n3c4ccccc4c4ccccc43)c2)c2cccc3c2-c2ccccc2C32c3ccccc3-c3ccccc32)cc1 ; c1ccc(-c2ccccc2-c2ccccc2-c2ccccc2N(c2ccccc2-n2c3ccccc3c3ccccc32)c2cccc3c2-c2ccccc2C32c3ccccc3-c3ccccc32)cc1 ; c1ccc(-c2ccccc2-c2ccccc2-c2ccccc2N(c2cccc(-c3cccc4c5ccccc5n(-c5ccccc5)c34)c2)c2cccc3c2-c2ccccc2C32c3ccccc3-c3ccccc32)cc1]

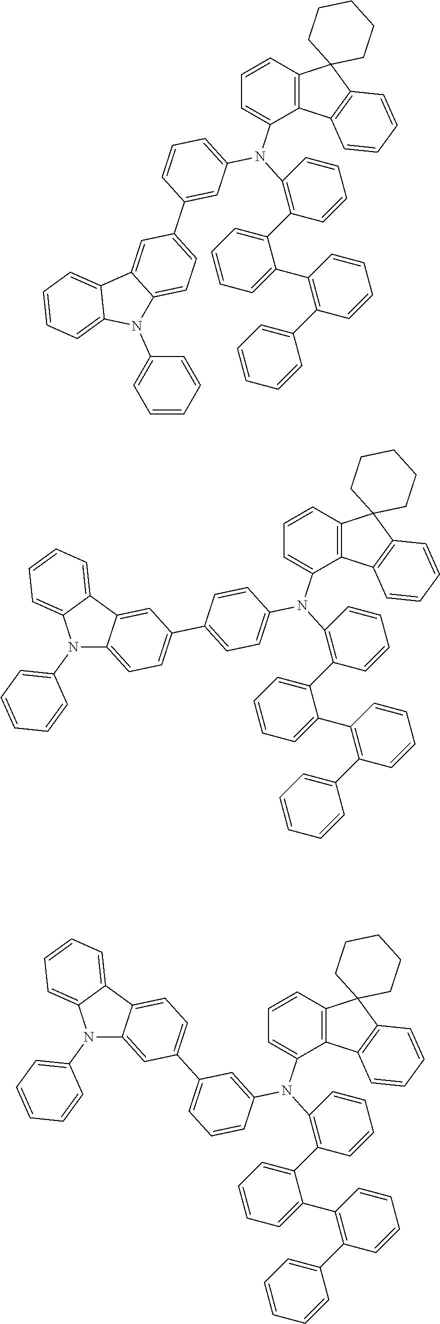 [SMILES: c1ccc(-c2ccccc2-c2ccccc2-c2ccccc2N(c2cccc(-c3ccc4c(c3)c3ccccc3n4-c3ccccc3)c2)c2cccc3c2-c2ccccc2C32CCCCC2)cc1 ; c1ccc(-c2ccccc2-c2ccccc2-c2ccccc2N(c2ccc(-c3ccc4c(c3)c3ccccc3n4-c3ccccc3)cc2)c2cccc3c2-c2ccccc2C32CCCCC2)cc1 ; c1ccc(-c2ccccc2-c2ccccc2-c2ccccc2N(c2cccc(-c3ccc4c5ccccc5n(-c5ccccc5)c4c3)c2)c2cccc3c2-c2ccccc2C32CCCCC2)cc1]

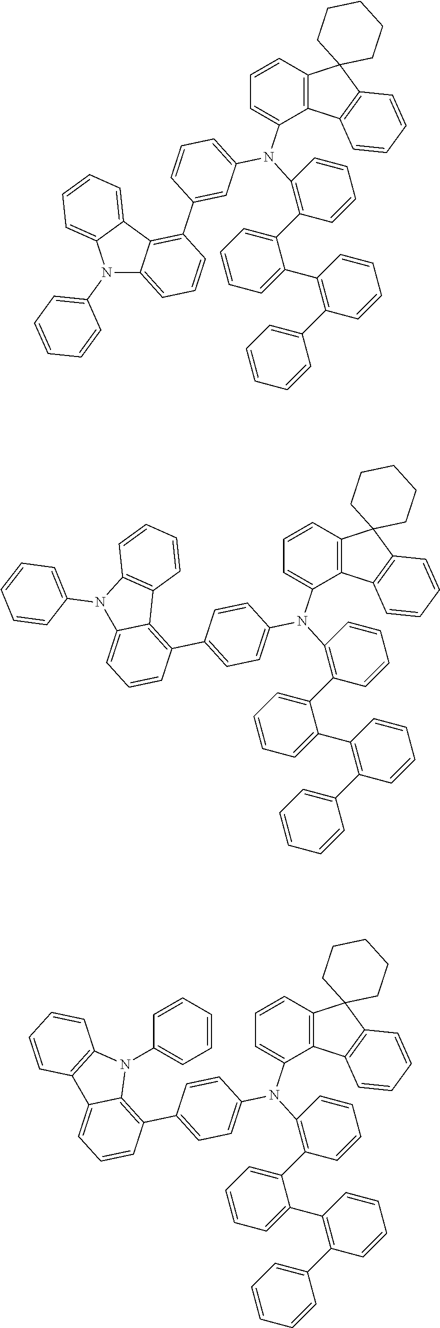 [SMILES: c1ccc(-c2ccccc2-c2ccccc2-c2ccccc2N(c2cccc(-c3cccc4c3c3ccccc3n4-c3ccccc3)c2)c2cccc3c2-c2ccccc2C32CCCCC2)cc1 ; c1ccc(-c2ccccc2-c2ccccc2-c2ccccc2N(c2ccc(-c3cccc4c3c3ccccc3n4-c3ccccc3)cc2)c2cccc3c2-c2ccccc2C32CCCCC2)cc1 ; c1ccc(-c2ccccc2-c2ccccc2-c2ccccc2N(c2ccc(-c3cccc4c5ccccc5n(-c5ccccc5)c34)cc2)c2cccc3c2-c2ccccc2C32CCCCC2)cc1]

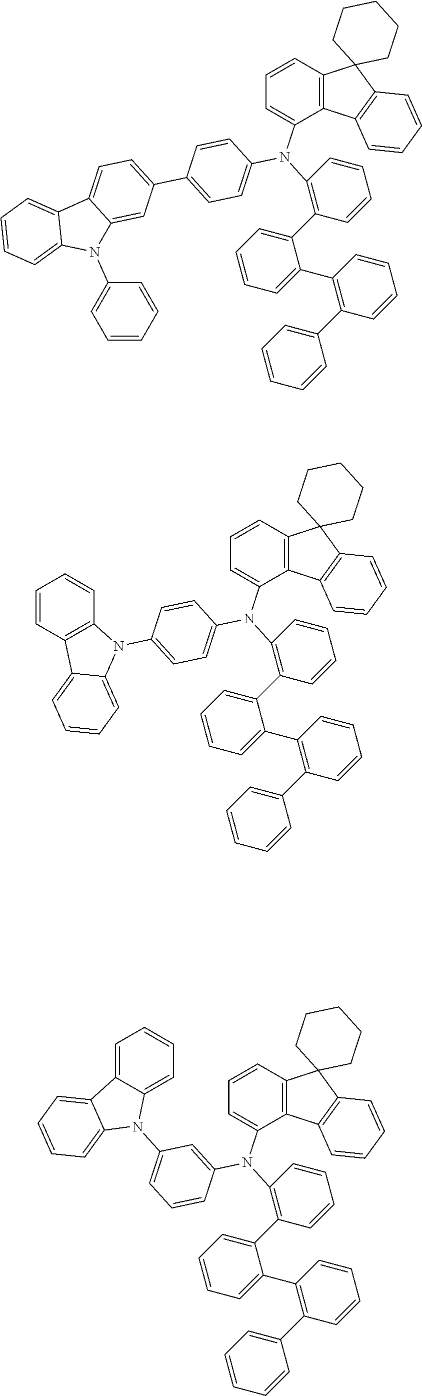 [SMILES: c1ccc(-c2ccccc2-c2ccccc2-c2ccccc2N(c2ccc(-c3ccc4c5ccccc5n(-c5ccccc5)c4c3)cc2)c2cccc3c2-c2ccccc2C32CCCCC2)cc1 ; c1ccc(-c2ccccc2-c2ccccc2-c2ccccc2N(c2ccc(-n3c4ccccc4c4ccccc43)cc2)c2cccc3c2-c2ccccc2C32CCCCC2)cc1 ; c1ccc(-c2ccccc2-c2ccccc2-c2ccccc2N(c2cccc(-n3c4ccccc4c4ccccc43)c2)c2cccc3c2-c2ccccc2C32CCCCC2)cc1]

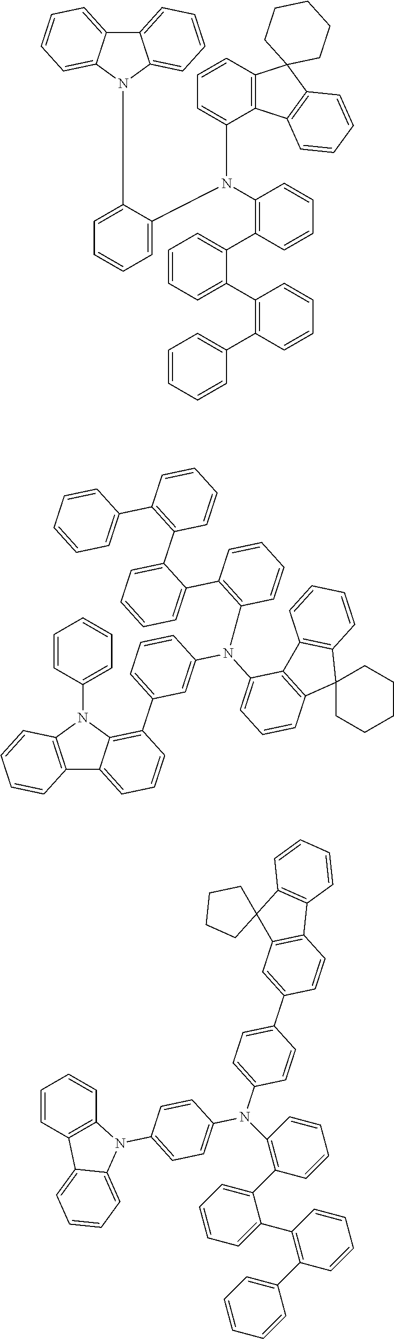 [SMILES: c1ccc(-c2ccccc2-c2ccccc2-c2ccccc2N(c2ccccc2-n2c3ccccc3c3ccccc32)c2cccc3c2-c2ccccc2C32CCCCC2)cc1 ; c1ccc(-c2ccccc2-c2ccccc2-c2ccccc2N(c2cccc(-c3cccc4c5ccccc5n(-c5ccccc5)c34)c2)c2cccc3c2-c2ccccc2C32CCCCC2)cc1 ; c1ccc(-c2ccccc2-c2ccccc2-c2ccccc2N(c2ccc(-c3ccc4c(c3)C3(CCCC3)c3ccccc3-4)cc2)c2ccc(-n3c4ccccc4c4ccccc43)cc2)cc1]

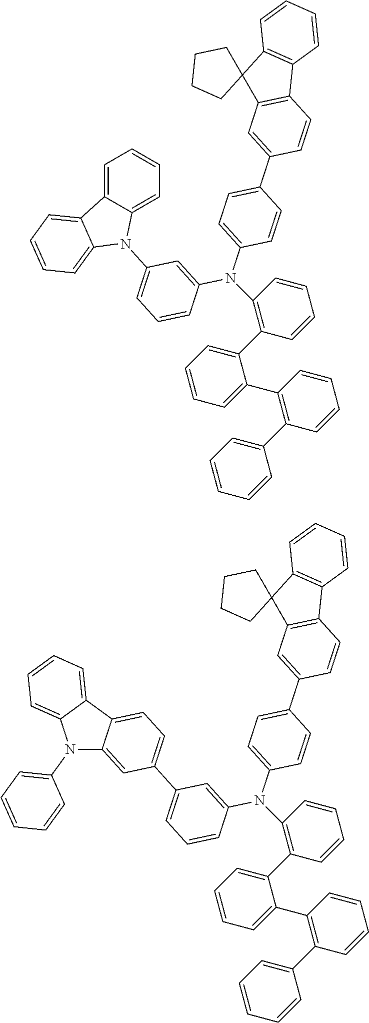 [SMILES: c1ccc(-c2ccccc2-c2ccccc2-c2ccccc2N(c2ccc(-c3ccc4c(c3)C3(CCCC3)c3ccccc3-4)cc2)c2cccc(-n3c4ccccc4c4ccccc43)c2)cc1 ; c1ccc(-c2ccccc2-c2ccccc2-c2ccccc2N(c2ccc(-c3ccc4c(c3)C3(CCCC3)c3ccccc3-4)cc2)c2cccc(-c3ccc4c5ccccc5n(-c5ccccc5)c4c3)c2)cc1]

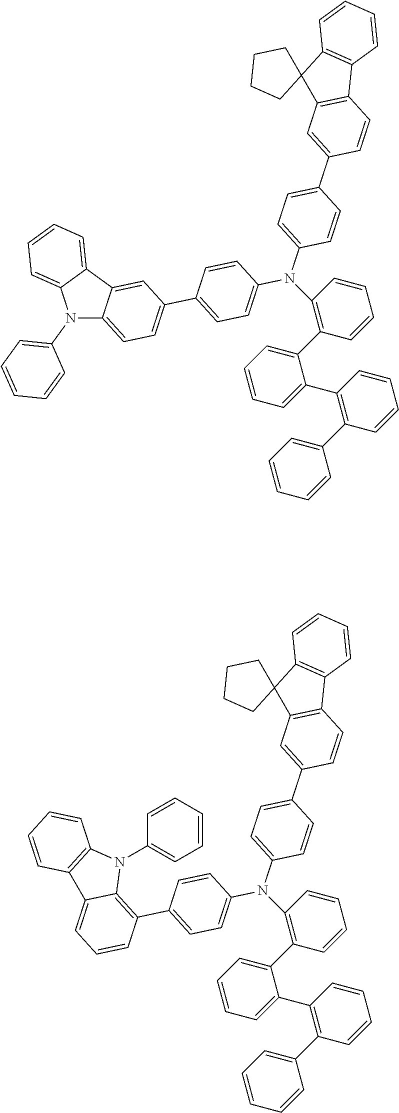 [SMILES: c1ccc(-c2ccccc2-c2ccccc2-c2ccccc2N(c2ccc(-c3ccc4c(c3)C3(CCCC3)c3ccccc3-4)cc2)c2ccc(-c3ccc4c(c3)c3ccccc3n4-c3ccccc3)cc2)cc1 ; c1ccc(-c2ccccc2-c2ccccc2-c2ccccc2N(c2ccc(-c3ccc4c(c3)C3(CCCC3)c3ccccc3-4)cc2)c2ccc(-c3cccc4c5ccccc5n(-c5ccccc5)c34)cc2)cc1]

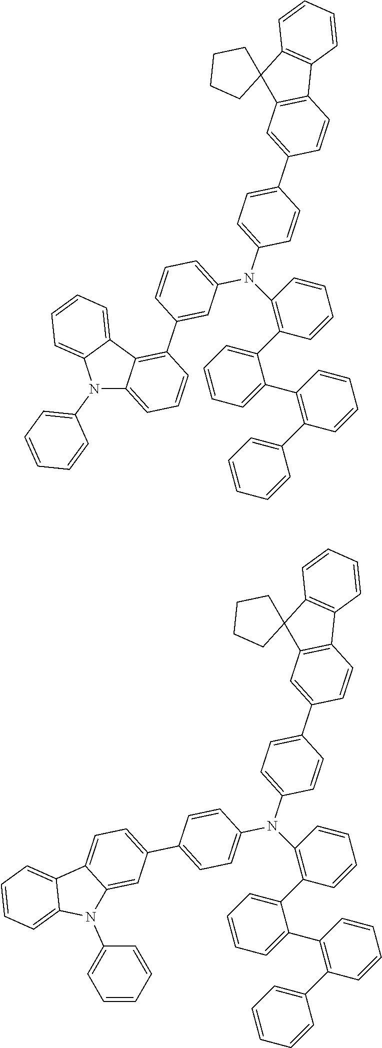 [SMILES: c1ccc(-c2ccccc2-c2ccccc2-c2ccccc2N(c2ccc(-c3ccc4c(c3)C3(CCCC3)c3ccccc3-4)cc2)c2cccc(-c3cccc4c3c3ccccc3n4-c3ccccc3)c2)cc1 ; c1ccc(-c2ccccc2-c2ccccc2-c2ccccc2N(c2ccc(-c3ccc4c(c3)C3(CCCC3)c3ccccc3-4)cc2)c2ccc(-c3ccc4c5ccccc5n(-c5ccccc5)c4c3)cc2)cc1]

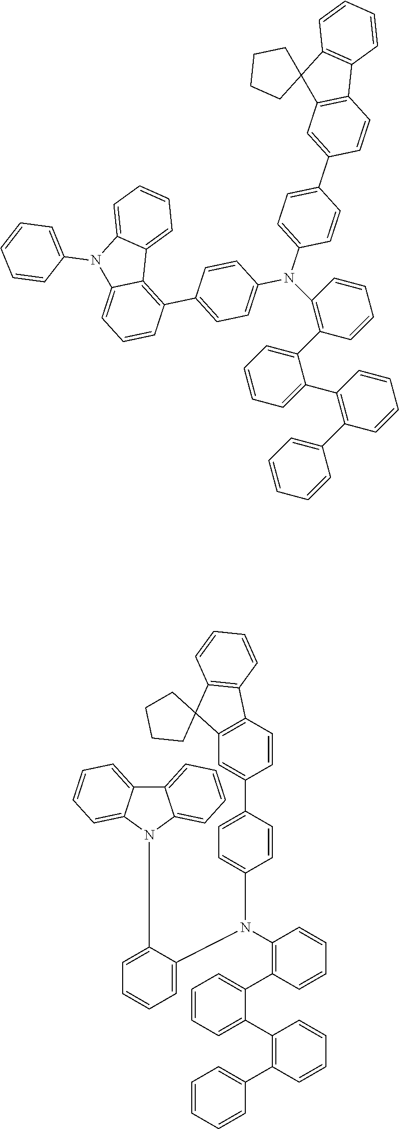 [SMILES: c1ccc(-c2ccccc2-c2ccccc2-c2ccccc2N(c2ccc(-c3ccc4c(c3)C3(CCCC3)c3ccccc3-4)cc2)c2ccc(-c3cccc4c3c3ccccc3n4-c3ccccc3)cc2)cc1 ; c1ccc(-c2ccccc2-c2ccccc2-c2ccccc2N(c2ccc(-c3ccc4c(c3)C3(CCCC3)c3ccccc3-4)cc2)c2ccccc2-n2c3ccccc3c3ccccc32)cc1]

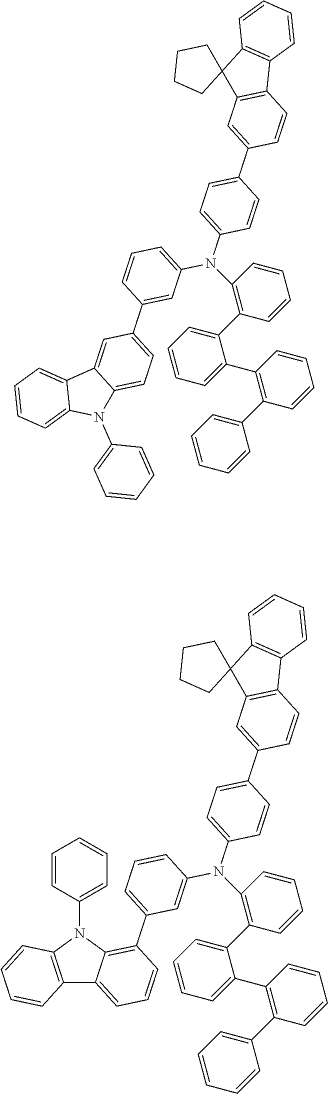 [SMILES: c1ccc(-c2ccccc2-c2ccccc2-c2ccccc2N(c2ccc(-c3ccc4c(c3)C3(CCCC3)c3ccccc3-4)cc2)c2cccc(-c3ccc4c(c3)c3ccccc3n4-c3ccccc3)c2)cc1 ; c1ccc(-c2ccccc2-c2ccccc2-c2ccccc2N(c2ccc(-c3ccc4c(c3)C3(CCCC3)c3ccccc3-4)cc2)c2cccc(-c3cccc4c5ccccc5n(-c5ccccc5)c34)c2)cc1]

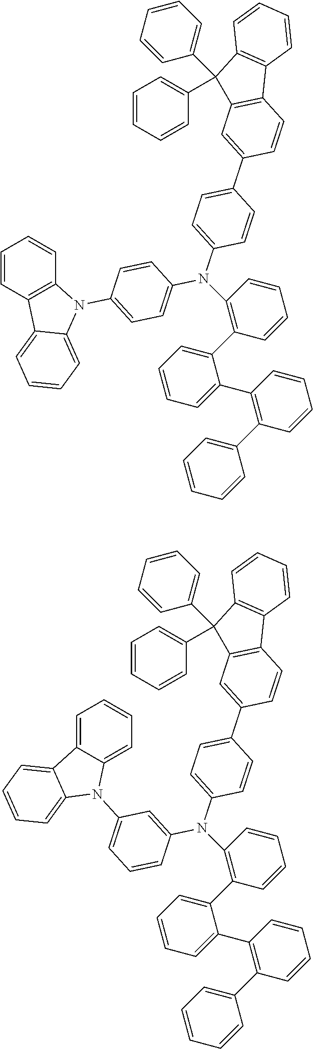 [SMILES: c1ccc(-c2ccccc2-c2ccccc2-c2ccccc2N(c2ccc(-c3ccc4c(c3)C(c3ccccc3)(c3ccccc3)c3ccccc3-4)cc2)c2ccc(-n3c4ccccc4c4ccccc43)cc2)cc1 ; c1ccc(-c2ccccc2-c2ccccc2-c2ccccc2N(c2ccc(-c3ccc4c(c3)C(c3ccccc3)(c3ccccc3)c3ccccc3-4)cc2)c2cccc(-n3c4ccccc4c4ccccc43)c2)cc1]

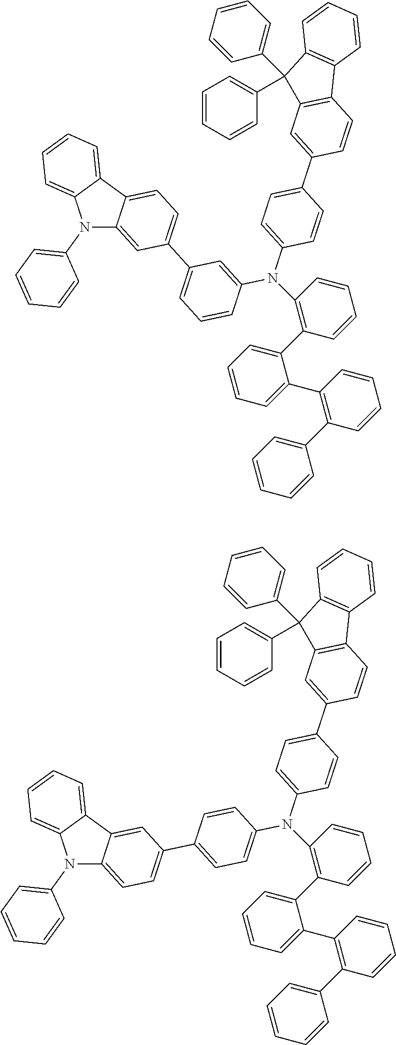 [SMILES: c1ccc(-c2ccccc2-c2ccccc2-c2ccccc2N(c2ccc(-c3ccc4c(c3)C(c3ccccc3)(c3ccccc3)c3ccccc3-4)cc2)c2cccc(-c3ccc4c5ccccc5n(-c5ccccc5)c4c3)c2)cc1 ; c1ccc(-c2ccccc2-c2ccccc2-c2ccccc2N(c2ccc(-c3ccc4c(c3)C(c3ccccc3)(c3ccccc3)c3ccccc3-4)cc2)c2ccc(-c3ccc4c(c3)c3ccccc3n4-c3ccccc3)cc2)cc1]

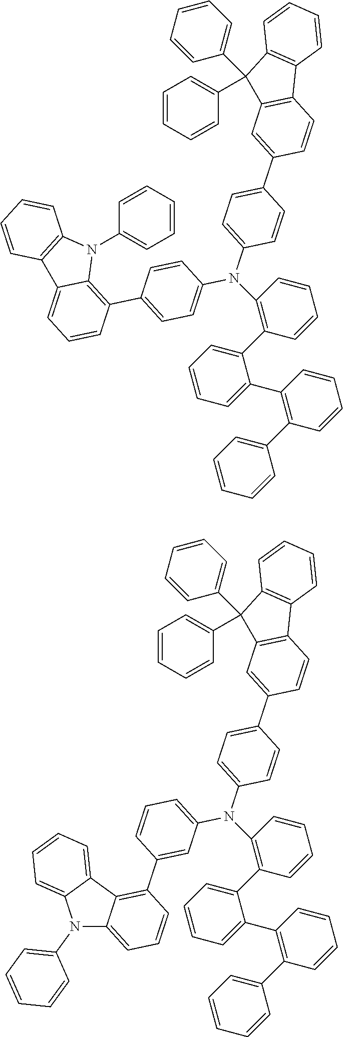 [SMILES: c1ccc(-c2ccccc2-c2ccccc2-c2ccccc2N(c2ccc(-c3ccc4c(c3)C(c3ccccc3)(c3ccccc3)c3ccccc3-4)cc2)c2ccc(-c3cccc4c5ccccc5n(-c5ccccc5)c34)cc2)cc1 ; c1ccc(-c2ccccc2-c2ccccc2-c2ccccc2N(c2ccc(-c3ccc4c(c3)C(c3ccccc3)(c3ccccc3)c3ccccc3-4)cc2)c2cccc(-c3cccc4c3c3ccccc3n4-c3ccccc3)c2)cc1]

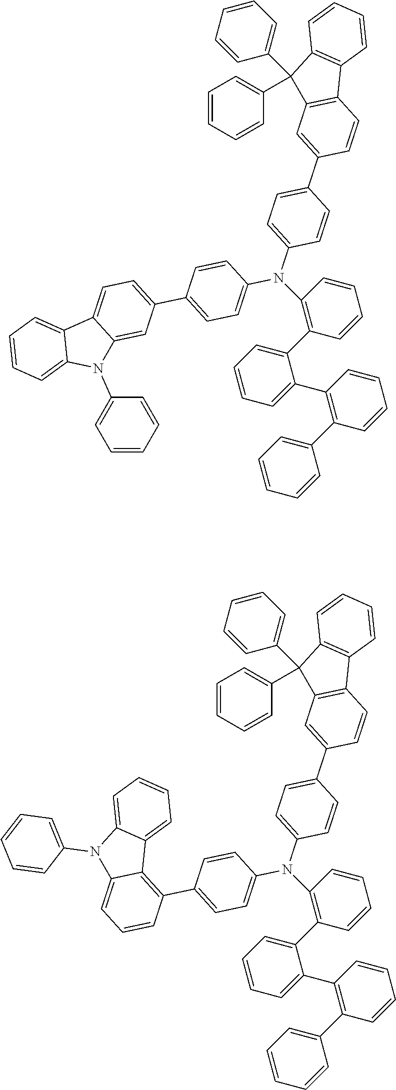 [SMILES: c1ccc(-c2ccccc2-c2ccccc2-c2ccccc2N(c2ccc(-c3ccc4c(c3)C(c3ccccc3)(c3ccccc3)c3ccccc3-4)cc2)c2ccc(-c3ccc4c5ccccc5n(-c5ccccc5)c4c3)cc2)cc1 ; c1ccc(-c2ccccc2-c2ccccc2-c2ccccc2N(c2ccc(-c3ccc4c(c3)C(c3ccccc3)(c3ccccc3)c3ccccc3-4)cc2)c2ccc(-c3cccc4c3c3ccccc3n4-c3ccccc3)cc2)cc1]

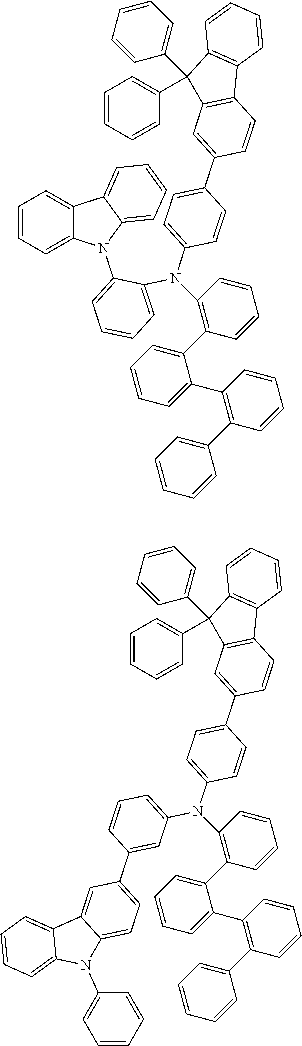 [SMILES: c1ccc(-c2ccccc2-c2ccccc2-c2ccccc2N(c2ccc(-c3ccc4c(c3)C(c3ccccc3)(c3ccccc3)c3ccccc3-4)cc2)c2ccccc2-n2c3ccccc3c3ccccc32)cc1 ; c1ccc(-c2ccccc2-c2ccccc2-c2ccccc2N(c2ccc(-c3ccc4c(c3)C(c3ccccc3)(c3ccccc3)c3ccccc3-4)cc2)c2cccc(-c3ccc4c(c3)c3ccccc3n4-c3ccccc3)c2)cc1]

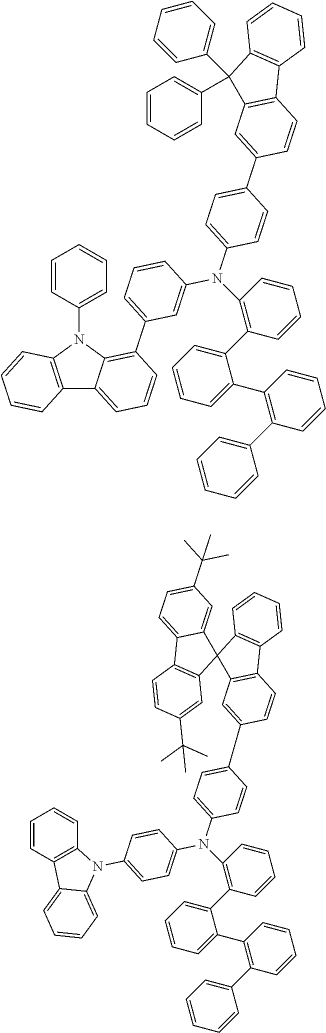 [SMILES: c1ccc(-c2ccccc2-c2ccccc2-c2ccccc2N(c2ccc(-c3ccc4c(c3)C(c3ccccc3)(c3ccccc3)c3ccccc3-4)cc2)c2cccc(-c3cccc4c5ccccc5n(-c5ccccc5)c34)c2)cc1 ; CC(C)(C)c1ccc2c(c1)C1(c3ccccc3-c3ccc(-c4ccc(N(c5ccc(-n6c7ccccc7c7ccccc76)cc5)c5ccccc5-c5ccccc5-c5ccccc5-c5ccccc5)cc4)cc31)c1cc(C(C)(C)C)ccc1-2]

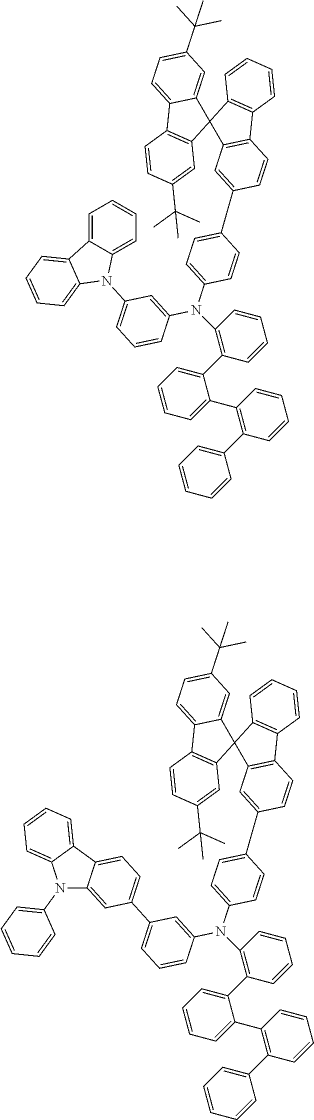 [SMILES: CC(C)(C)c1ccc2c(c1)C1(c3ccccc3-c3ccc(-c4ccc(N(c5cccc(-n6c7ccccc7c7ccccc76)c5)c5ccccc5-c5ccccc5-c5ccccc5-c5ccccc5)cc4)cc31)c1cc(C(C)(C)C)ccc1-2 ; CC(C)(C)c1ccc2c(c1)C1(c3ccccc3-c3ccc(-c4ccc(N(c5cccc(-c6ccc7c8ccccc8n(-c8ccccc8)c7c6)c5)c5ccccc5-c5ccccc5-c5ccccc5-c5ccccc5)cc4)cc31)c1cc(C(C)(C)C)ccc1-2]

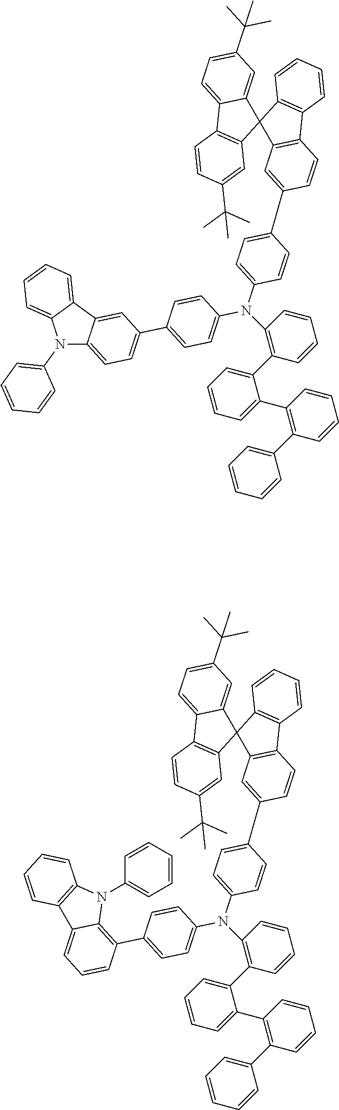 [SMILES: CC(C)(C)c1ccc2c(c1)C1(c3ccccc3-c3ccc(-c4ccc(N(c5ccc(-c6ccc7c(c6)c6ccccc6n7-c6ccccc6)cc5)c5ccccc5-c5ccccc5-c5ccccc5-c5ccccc5)cc4)cc31)c1cc(C(C)(C)C)ccc1-2 ; CC(C)(C)c1ccc2c(c1)C1(c3ccccc3-c3ccc(-c4ccc(N(c5ccc(-c6cccc7c8ccccc8n(-c8ccccc8)c67)cc5)c5ccccc5-c5ccccc5-c5ccccc5-c5ccccc5)cc4)cc31)c1cc(C(C)(C)C)ccc1-2]

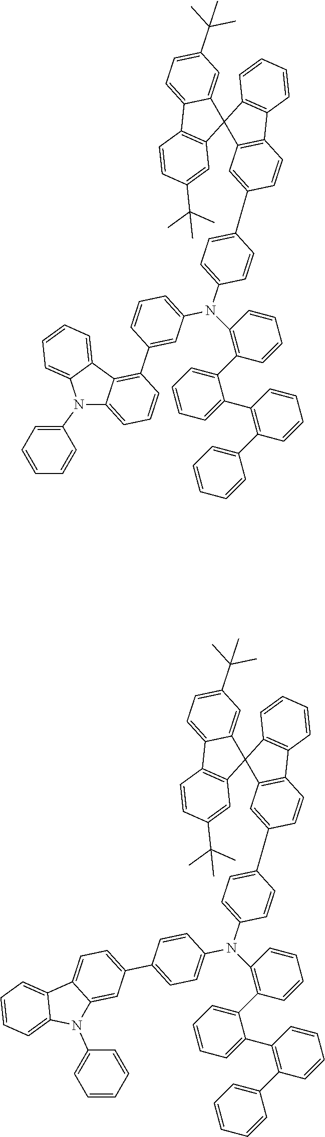 [SMILES: CC(C)(C)c1ccc2c(c1)C1(c3ccccc3-c3ccc(-c4ccc(N(c5cccc(-c6cccc7c6c6ccccc6n7-c6ccccc6)c5)c5ccccc5-c5ccccc5-c5ccccc5-c5ccccc5)cc4)cc31)c1cc(C(C)(C)C)ccc1-2 ; CC(C)(C)c1ccc2c(c1)C1(c3ccccc3-c3ccc(-c4ccc(N(c5ccc(-c6ccc7c8ccccc8n(-c8ccccc8)c7c6)cc5)c5ccccc5-c5ccccc5-c5ccccc5-c5ccccc5)cc4)cc31)c1cc(C(C)(C)C)ccc1-2]

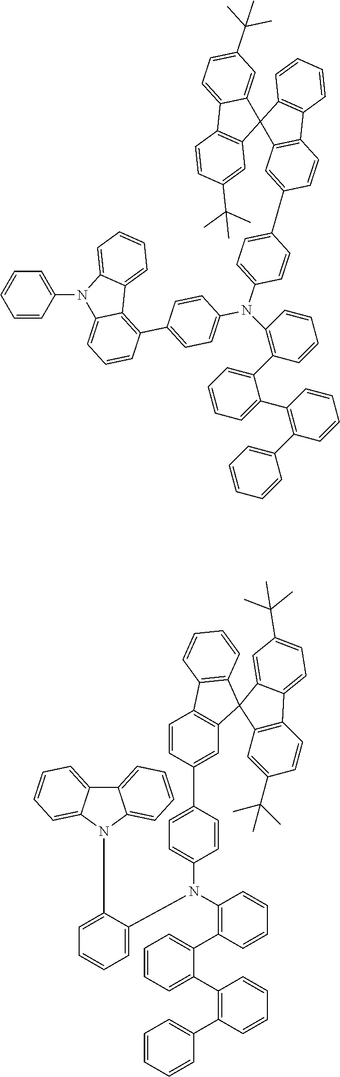 [SMILES: CC(C)(C)c1ccc2c(c1)C1(c3ccccc3-c3ccc(-c4ccc(N(c5ccc(-c6cccc7c6c6ccccc6n7-c6ccccc6)cc5)c5ccccc5-c5ccccc5-c5ccccc5-c5ccccc5)cc4)cc31)c1cc(C(C)(C)C)ccc1-2 ; CC(C)(C)c1ccc2c(c1)C1(c3ccccc3-c3ccc(-c4ccc(N(c5ccccc5-c5ccccc5-c5ccccc5-c5ccccc5)c5ccccc5-n5c6ccccc6c6ccccc65)cc4)cc31)c1cc(C(C)(C)C)ccc1-2]

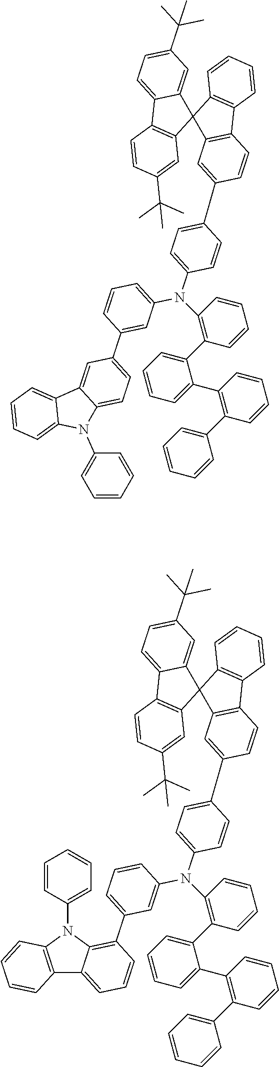 [SMILES: CC(C)(C)c1ccc2c(c1)C1(c3ccccc3-c3ccc(-c4ccc(N(c5cccc(-c6ccc7c(c6)c6ccccc6n7-c6ccccc6)c5)c5ccccc5-c5ccccc5-c5ccccc5-c5ccccc5)cc4)cc31)c1cc(C(C)(C)C)ccc1-2 ; CC(C)(C)c1ccc2c(c1)C1(c3ccccc3-c3ccc(-c4ccc(N(c5cccc(-c6cccc7c8ccccc8n(-c8ccccc8)c67)c5)c5ccccc5-c5ccccc5-c5ccccc5-c5ccccc5)cc4)cc31)c1cc(C(C)(C)C)ccc1-2]

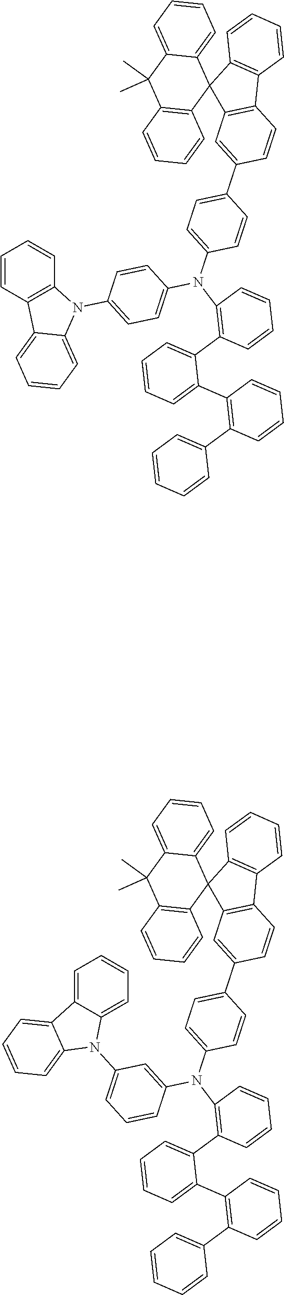 [SMILES: CC1(C)c2ccccc2C2(c3ccccc3-c3ccc(-c4ccc(N(c5ccc(-n6c7ccccc7c7ccccc76)cc5)c5ccccc5-c5ccccc5-c5ccccc5-c5ccccc5)cc4)cc32)c2ccccc21 ; CC1(C)c2ccccc2C2(c3ccccc3-c3ccc(-c4ccc(N(c5cccc(-n6c7ccccc7c7ccccc76)c5)c5ccccc5-c5ccccc5-c5ccccc5-c5ccccc5)cc4)cc32)c2ccccc21]

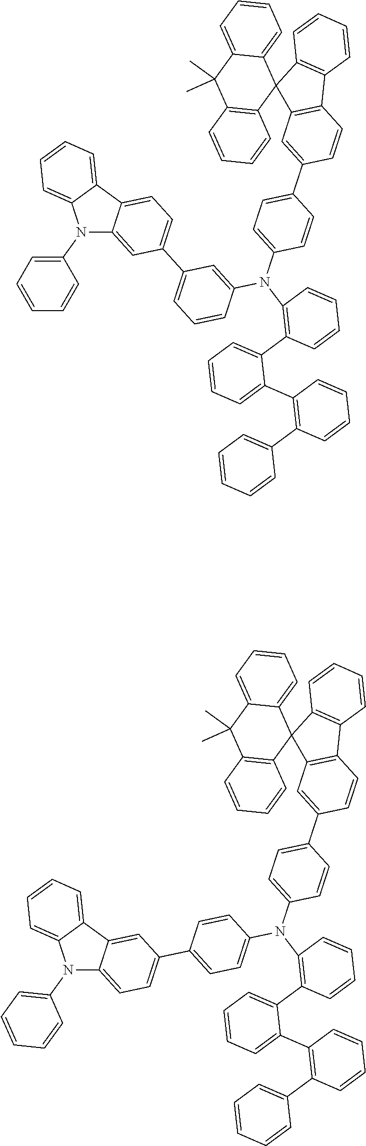 [SMILES: CC1(C)c2ccccc2C2(c3ccccc3-c3ccc(-c4ccc(N(c5cccc(-c6ccc7c8ccccc8n(-c8ccccc8)c7c6)c5)c5ccccc5-c5ccccc5-c5ccccc5-c5ccccc5)cc4)cc32)c2ccccc21 ; CC1(C)c2ccccc2C2(c3ccccc3-c3ccc(-c4ccc(N(c5ccc(-c6ccc7c(c6)c6ccccc6n7-c6ccccc6)cc5)c5ccccc5-c5ccccc5-c5ccccc5-c5ccccc5)cc4)cc32)c2ccccc21]

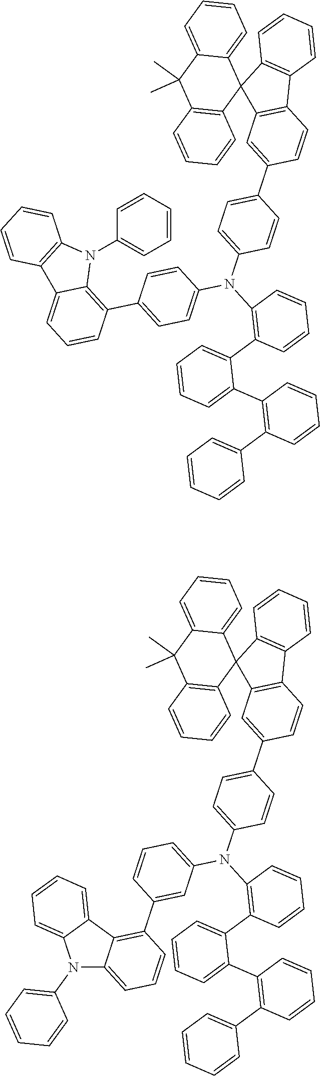 [SMILES: CC1(C)c2ccccc2C2(c3ccccc3-c3ccc(-c4ccc(N(c5ccc(-c6cccc7c8ccccc8n(-c8ccccc8)c67)cc5)c5ccccc5-c5ccccc5-c5ccccc5-c5ccccc5)cc4)cc32)c2ccccc21 ; CC1(C)c2ccccc2C2(c3ccccc3-c3ccc(-c4ccc(N(c5cccc(-c6cccc7c6c6ccccc6n7-c6ccccc6)c5)c5ccccc5-c5ccccc5-c5ccccc5-c5ccccc5)cc4)cc32)c2ccccc21]

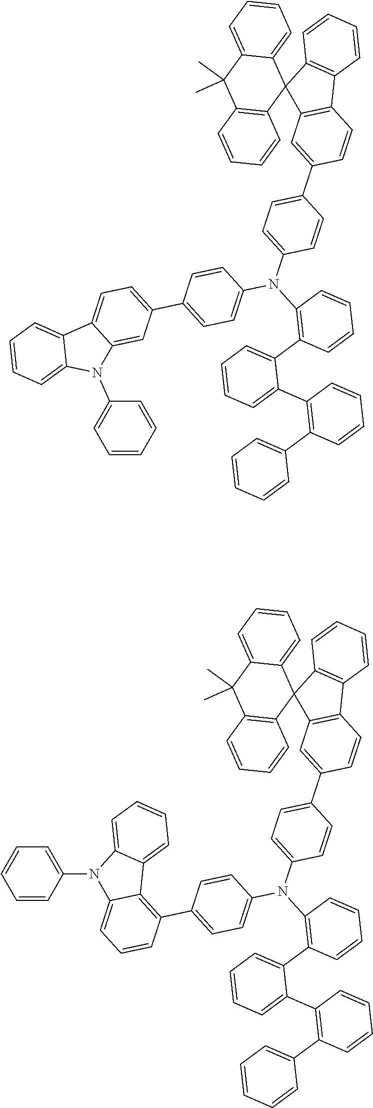 [SMILES: CC1(C)c2ccccc2C2(c3ccccc3-c3ccc(-c4ccc(N(c5ccc(-c6ccc7c8ccccc8n(-c8ccccc8)c7c6)cc5)c5ccccc5-c5ccccc5-c5ccccc5-c5ccccc5)cc4)cc32)c2ccccc21 ; CC1(C)c2ccccc2C2(c3ccccc3-c3ccc(-c4ccc(N(c5ccc(-c6cccc7c6c6ccccc6n7-c6ccccc6)cc5)c5ccccc5-c5ccccc5-c5ccccc5-c5ccccc5)cc4)cc32)c2ccccc21]

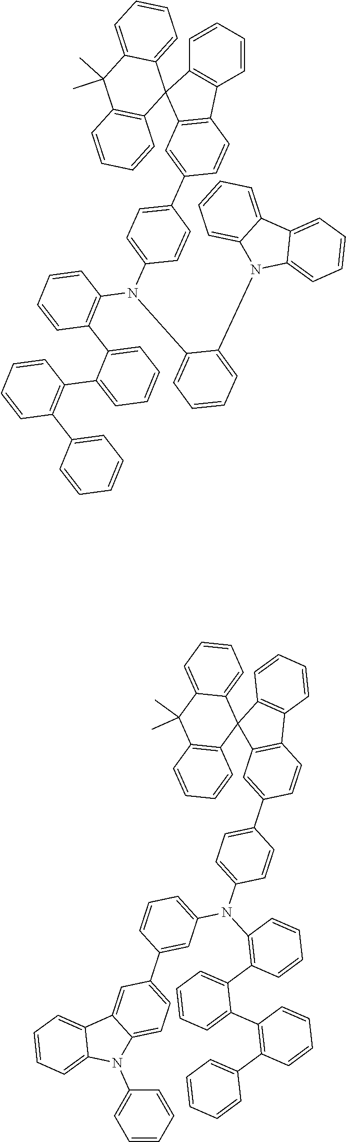 [SMILES: CC1(C)c2ccccc2C2(c3ccccc3-c3ccc(-c4ccc(N(c5ccccc5-c5ccccc5-c5ccccc5-c5ccccc5)c5ccccc5-n5c6ccccc6c6ccccc65)cc4)cc32)c2ccccc21 ; CC1(C)c2ccccc2C2(c3ccccc3-c3ccc(-c4ccc(N(c5cccc(-c6ccc7c(c6)c6ccccc6n7-c6ccccc6)c5)c5ccccc5-c5ccccc5-c5ccccc5-c5ccccc5)cc4)cc32)c2ccccc21]

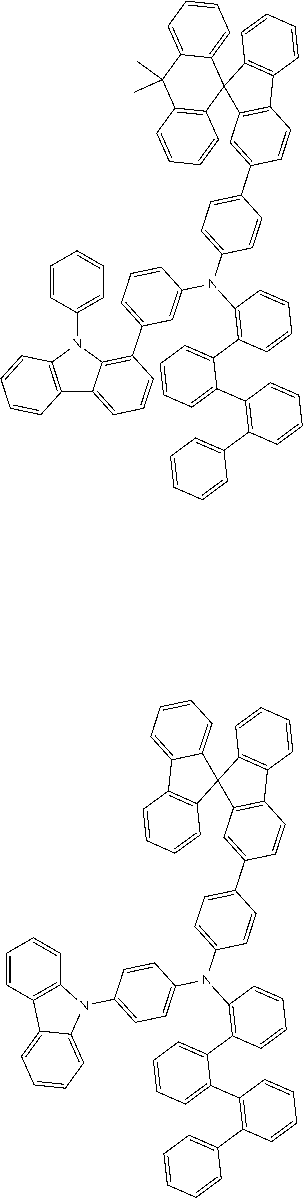 [SMILES: CC1(C)c2ccccc2C2(c3ccccc3-c3ccc(-c4ccc(N(c5cccc(-c6cccc7c8ccccc8n(-c8ccccc8)c67)c5)c5ccccc5-c5ccccc5-c5ccccc5-c5ccccc5)cc4)cc32)c2ccccc21 ; c1ccc(-c2ccccc2-c2ccccc2-c2ccccc2N(c2ccc(-c3ccc4c(c3)C3(c5ccccc5-c5ccccc53)c3ccccc3-4)cc2)c2ccc(-n3c4ccccc4c4ccccc43)cc2)cc1]

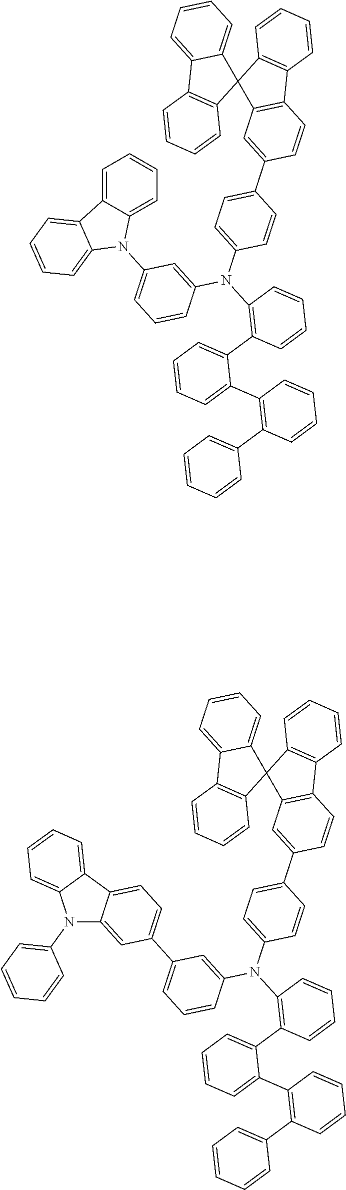 [SMILES: c1ccc(-c2ccccc2-c2ccccc2-c2ccccc2N(c2ccc(-c3ccc4c(c3)C3(c5ccccc5-c5ccccc53)c3ccccc3-4)cc2)c2cccc(-n3c4ccccc4c4ccccc43)c2)cc1 ; c1ccc(-c2ccccc2-c2ccccc2-c2ccccc2N(c2ccc(-c3ccc4c(c3)C3(c5ccccc5-c5ccccc53)c3ccccc3-4)cc2)c2cccc(-c3ccc4c5ccccc5n(-c5ccccc5)c4c3)c2)cc1]

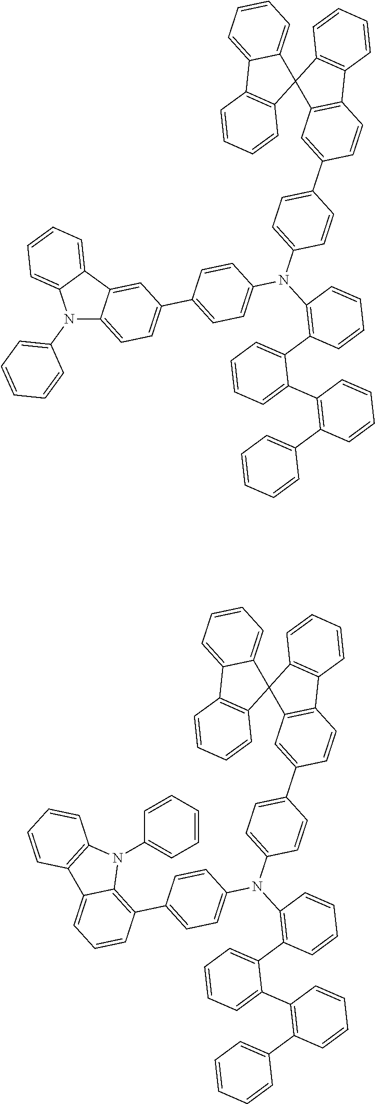 [SMILES: c1ccc(-c2ccccc2-c2ccccc2-c2ccccc2N(c2ccc(-c3ccc4c(c3)C3(c5ccccc5-c5ccccc53)c3ccccc3-4)cc2)c2ccc(-c3ccc4c(c3)c3ccccc3n4-c3ccccc3)cc2)cc1 ; c1ccc(-c2ccccc2-c2ccccc2-c2ccccc2N(c2ccc(-c3ccc4c(c3)C3(c5ccccc5-c5ccccc53)c3ccccc3-4)cc2)c2ccc(-c3cccc4c5ccccc5n(-c5ccccc5)c34)cc2)cc1]

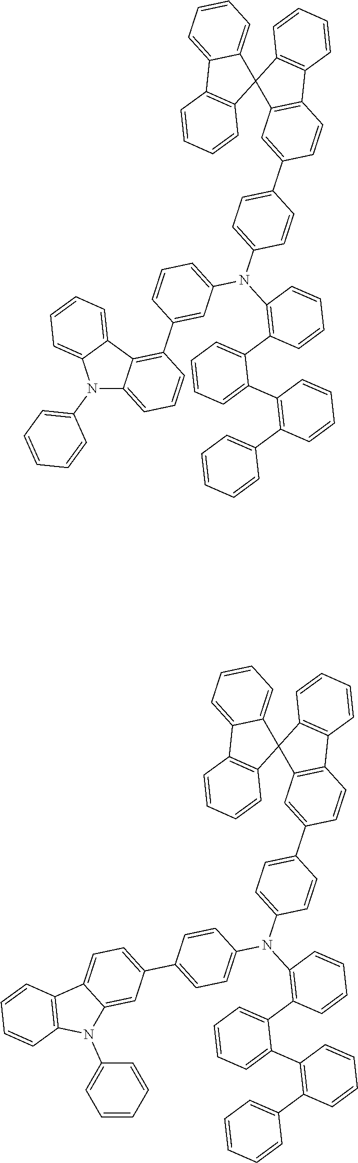 [SMILES: c1ccc(-c2ccccc2-c2ccccc2-c2ccccc2N(c2ccc(-c3ccc4c(c3)C3(c5ccccc5-c5ccccc53)c3ccccc3-4)cc2)c2cccc(-c3cccc4c3c3ccccc3n4-c3ccccc3)c2)cc1 ; c1ccc(-c2ccccc2-c2ccccc2-c2ccccc2N(c2ccc(-c3ccc4c(c3)C3(c5ccccc5-c5ccccc53)c3ccccc3-4)cc2)c2ccc(-c3ccc4c5ccccc5n(-c5ccccc5)c4c3)cc2)cc1]

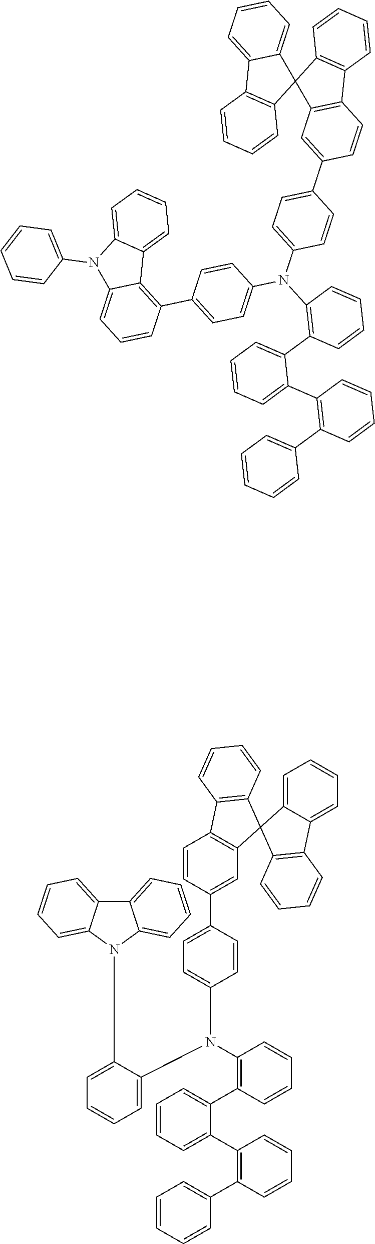 [SMILES: c1ccc(-c2ccccc2-c2ccccc2-c2ccccc2N(c2ccc(-c3ccc4c(c3)C3(c5ccccc5-c5ccccc53)c3ccccc3-4)cc2)c2ccc(-c3cccc4c3c3ccccc3n4-c3ccccc3)cc2)cc1 ; c1ccc(-c2ccccc2-c2ccccc2-c2ccccc2N(c2ccc(-c3ccc4c(c3)C3(c5ccccc5-c5ccccc53)c3ccccc3-4)cc2)c2ccccc2-n2c3ccccc3c3ccccc32)cc1]

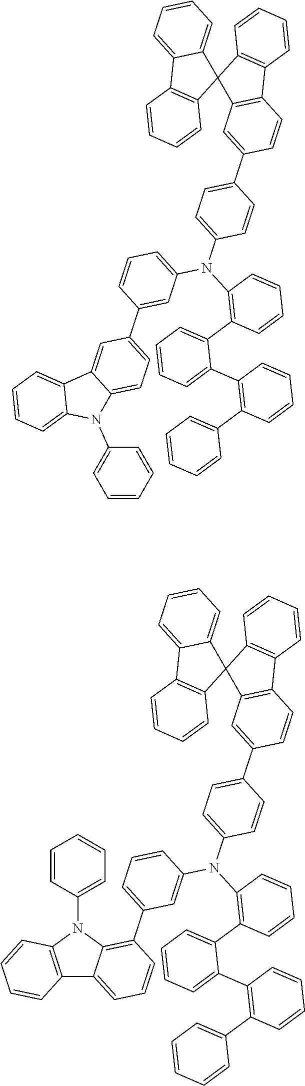 [SMILES: c1ccc(-c2ccccc2-c2ccccc2-c2ccccc2N(c2ccc(-c3ccc4c(c3)C3(c5ccccc5-c5ccccc53)c3ccccc3-4)cc2)c2cccc(-c3ccc4c(c3)c3ccccc3n4-c3ccccc3)c2)cc1 ; c1ccc(-c2ccccc2-c2ccccc2-c2ccccc2N(c2ccc(-c3ccc4c(c3)C3(c5ccccc5-c5ccccc53)c3ccccc3-4)cc2)c2cccc(-c3cccc4c5ccccc5n(-c5ccccc5)c34)c2)cc1]

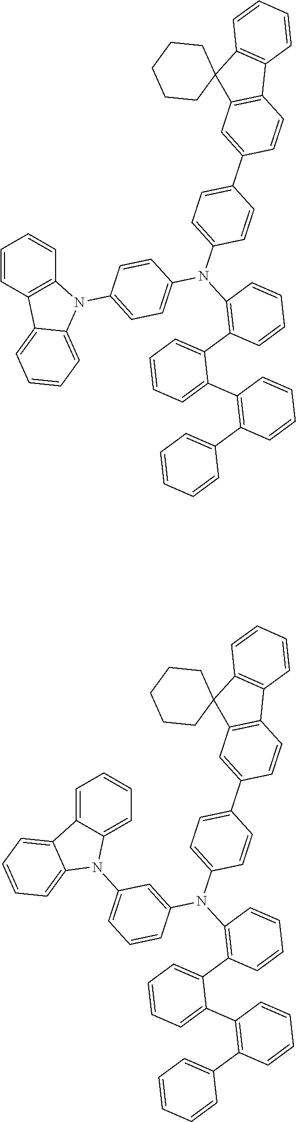 [SMILES: c1ccc(-c2ccccc2-c2ccccc2-c2ccccc2N(c2ccc(-c3ccc4c(c3)C3(CCCCC3)c3ccccc3-4)cc2)c2ccc(-n3c4ccccc4c4ccccc43)cc2)cc1 ; c1ccc(-c2ccccc2-c2ccccc2-c2ccccc2N(c2ccc(-c3ccc4c(c3)C3(CCCCC3)c3ccccc3-4)cc2)c2cccc(-n3c4ccccc4c4ccccc43)c2)cc1]

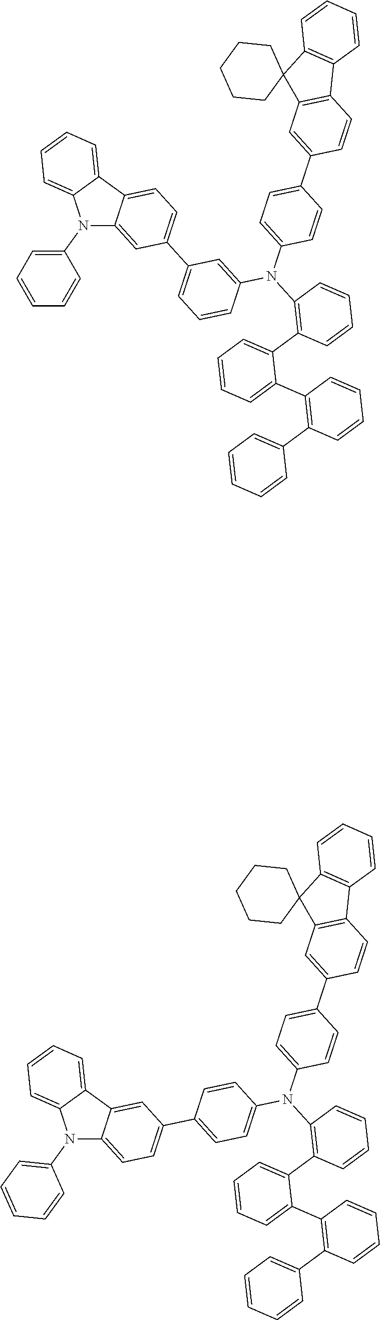 [SMILES: c1ccc(-c2ccccc2-c2ccccc2-c2ccccc2N(c2ccc(-c3ccc4c(c3)C3(CCCCC3)c3ccccc3-4)cc2)c2cccc(-c3ccc4c5ccccc5n(-c5ccccc5)c4c3)c2)cc1 ; c1ccc(-c2ccccc2-c2ccccc2-c2ccccc2N(c2ccc(-c3ccc4c(c3)C3(CCCCC3)c3ccccc3-4)cc2)c2ccc(-c3ccc4c(c3)c3ccccc3n4-c3ccccc3)cc2)cc1]

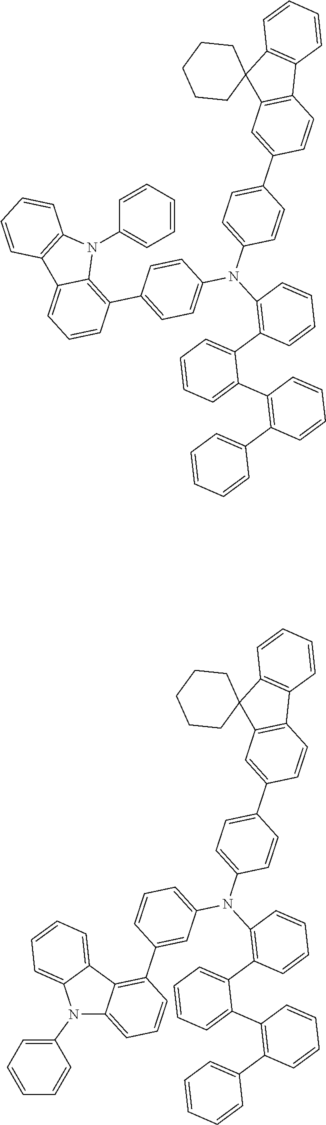 [SMILES: c1ccc(-c2ccccc2-c2ccccc2-c2ccccc2N(c2ccc(-c3ccc4c(c3)C3(CCCCC3)c3ccccc3-4)cc2)c2ccc(-c3cccc4c5ccccc5n(-c5ccccc5)c34)cc2)cc1 ; c1ccc(-c2ccccc2-c2ccccc2-c2ccccc2N(c2ccc(-c3ccc4c(c3)C3(CCCCC3)c3ccccc3-4)cc2)c2cccc(-c3cccc4c3c3ccccc3n4-c3ccccc3)c2)cc1]

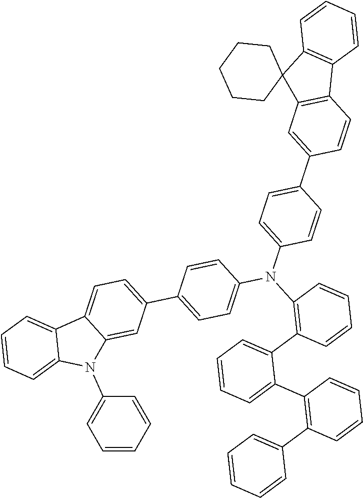 [SMILES: c1ccc(-c2ccccc2-c2ccccc2-c2ccccc2N(c2ccc(-c3ccc4c(c3)C3(CCCCC3)c3ccccc3-4)cc2)c2ccc(-c3ccc4c5ccccc5n(-c5ccccc5)c4c3)cc2)cc1]

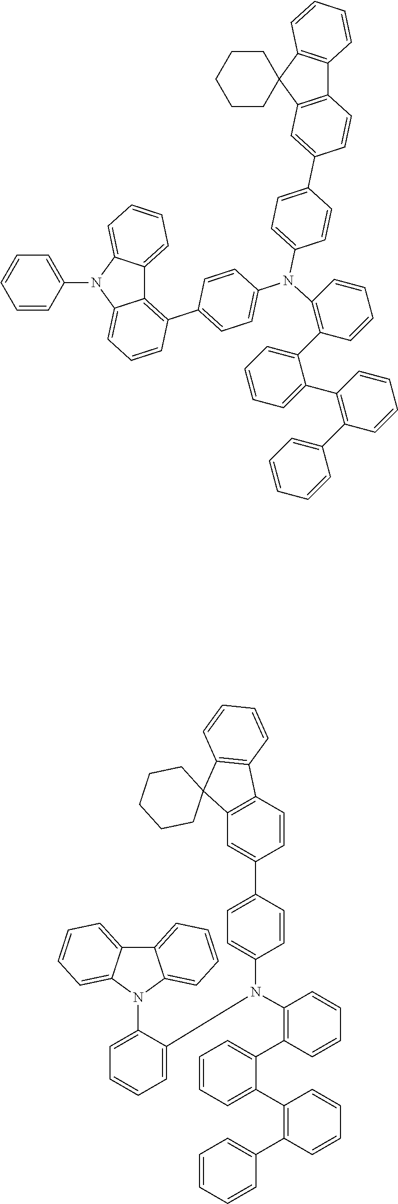 [SMILES: c1ccc(-c2ccccc2-c2ccccc2-c2ccccc2N(c2ccc(-c3ccc4c(c3)C3(CCCCC3)c3ccccc3-4)cc2)c2ccc(-c3cccc4c3c3ccccc3n4-c3ccccc3)cc2)cc1 ; c1ccc(-c2ccccc2-c2ccccc2-c2ccccc2N(c2ccc(-c3ccc4c(c3)C3(CCCCC3)c3ccccc3-4)cc2)c2ccccc2-n2c3ccccc3c3ccccc32)cc1]

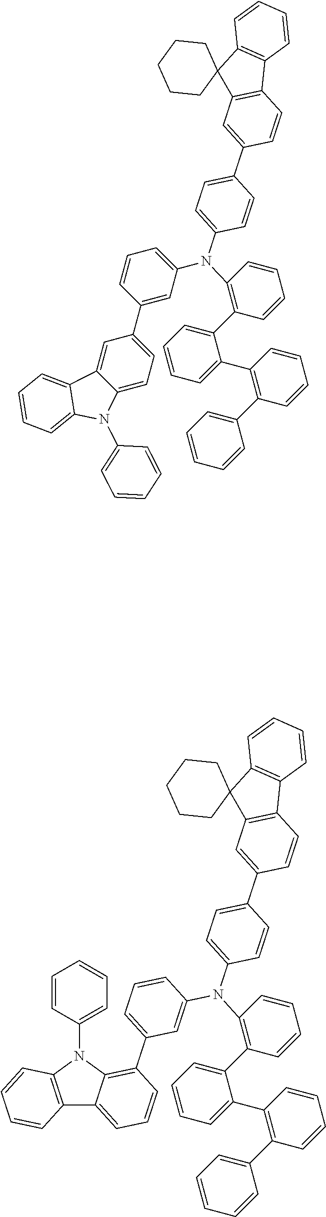 [SMILES: c1ccc(-c2ccccc2-c2ccccc2-c2ccccc2N(c2ccc(-c3ccc4c(c3)C3(CCCCC3)c3ccccc3-4)cc2)c2cccc(-c3ccc4c(c3)c3ccccc3n4-c3ccccc3)c2)cc1 ; c1ccc(-c2ccccc2-c2ccccc2-c2ccccc2N(c2ccc(-c3ccc4c(c3)C3(CCCCC3)c3ccccc3-4)cc2)c2cccc(-c3cccc4c5ccccc5n(-c5ccccc5)c34)c2)cc1]

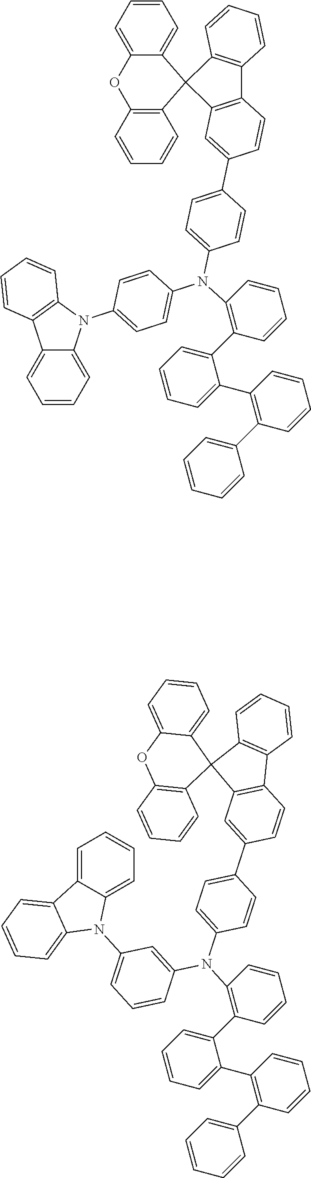 [SMILES: c1ccc(-c2ccccc2-c2ccccc2-c2ccccc2N(c2ccc(-c3ccc4c(c3)C3(c5ccccc5Oc5ccccc53)c3ccccc3-4)cc2)c2ccc(-n3c4ccccc4c4ccccc43)cc2)cc1 ; c1ccc(-c2ccccc2-c2ccccc2-c2ccccc2N(c2ccc(-c3ccc4c(c3)C3(c5ccccc5Oc5ccccc53)c3ccccc3-4)cc2)c2cccc(-n3c4ccccc4c4ccccc43)c2)cc1]

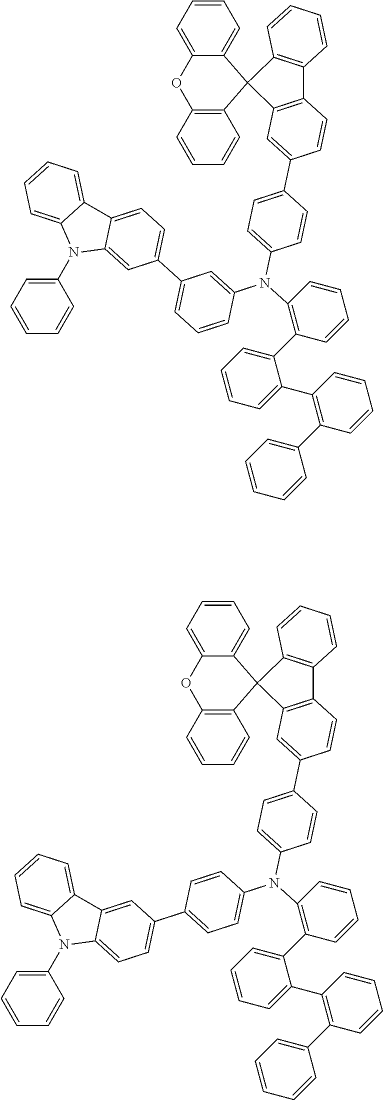 [SMILES: c1ccc(-c2ccccc2-c2ccccc2-c2ccccc2N(c2ccc(-c3ccc4c(c3)C3(c5ccccc5Oc5ccccc53)c3ccccc3-4)cc2)c2cccc(-c3ccc4c5ccccc5n(-c5ccccc5)c4c3)c2)cc1 ; c1ccc(-c2ccccc2-c2ccccc2-c2ccccc2N(c2ccc(-c3ccc4c(c3)C3(c5ccccc5Oc5ccccc53)c3ccccc3-4)cc2)c2ccc(-c3ccc4c(c3)c3ccccc3n4-c3ccccc3)cc2)cc1]

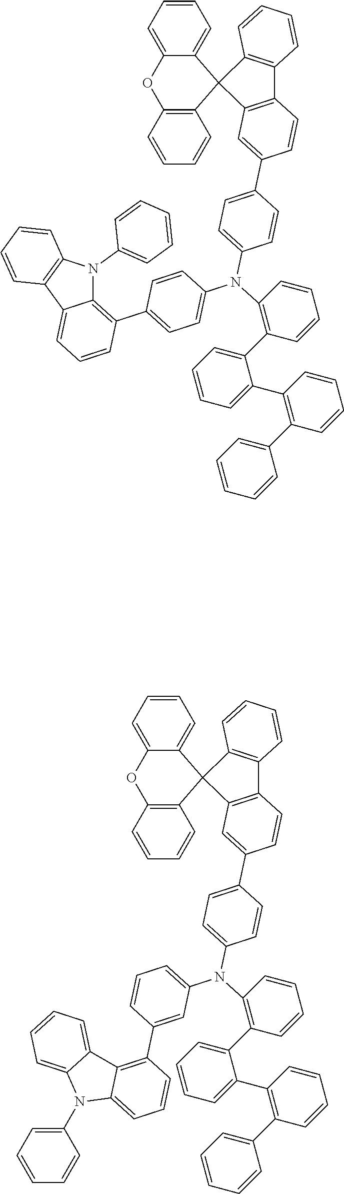 [SMILES: c1ccc(-c2ccccc2-c2ccccc2-c2ccccc2N(c2ccc(-c3ccc4c(c3)C3(c5ccccc5Oc5ccccc53)c3ccccc3-4)cc2)c2ccc(-c3cccc4c5ccccc5n(-c5ccccc5)c34)cc2)cc1 ; c1ccc(-c2ccccc2-c2ccccc2-c2ccccc2N(c2ccc(-c3ccc4c(c3)C3(c5ccccc5Oc5ccccc53)c3ccccc3-4)cc2)c2cccc(-c3cccc4c3c3ccccc3n4-c3ccccc3)c2)cc1]

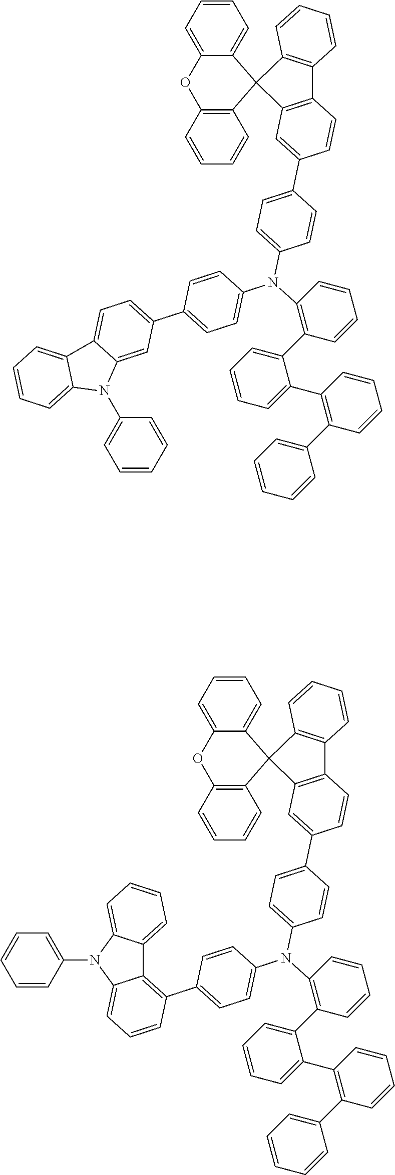 [SMILES: c1ccc(-c2ccccc2-c2ccccc2-c2ccccc2N(c2ccc(-c3ccc4c(c3)C3(c5ccccc5Oc5ccccc53)c3ccccc3-4)cc2)c2ccc(-c3ccc4c5ccccc5n(-c5ccccc5)c4c3)cc2)cc1 ; c1ccc(-c2ccccc2-c2ccccc2-c2ccccc2N(c2ccc(-c3ccc4c(c3)C3(c5ccccc5Oc5ccccc53)c3ccccc3-4)cc2)c2ccc(-c3cccc4c3c3ccccc3n4-c3ccccc3)cc2)cc1]

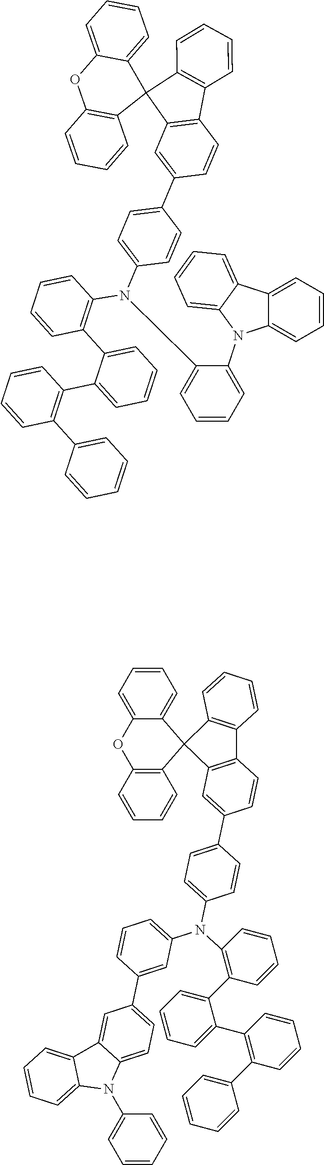 [SMILES: c1ccc(-c2ccccc2-c2ccccc2-c2ccccc2N(c2ccc(-c3ccc4c(c3)C3(c5ccccc5Oc5ccccc53)c3ccccc3-4)cc2)c2ccccc2-n2c3ccccc3c3ccccc32)cc1 ; c1ccc(-c2ccccc2-c2ccccc2-c2ccccc2N(c2ccc(-c3ccc4c(c3)C3(c5ccccc5Oc5ccccc53)c3ccccc3-4)cc2)c2cccc(-c3ccc4c(c3)c3ccccc3n4-c3ccccc3)c2)cc1]

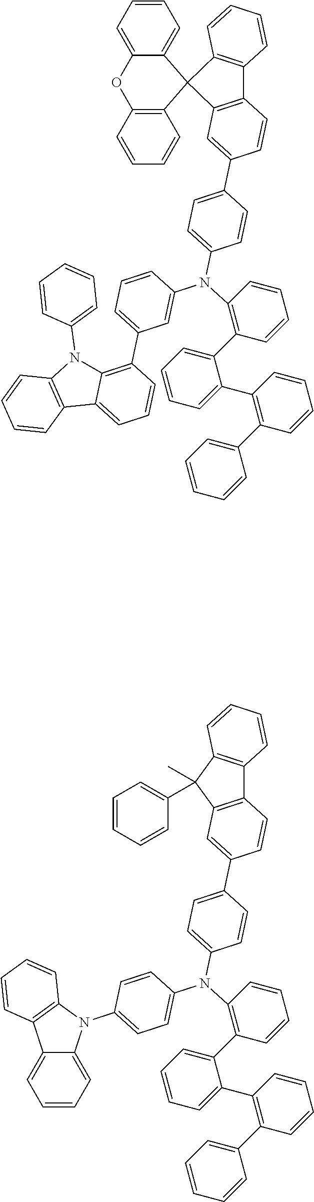 [SMILES: c1ccc(-c2ccccc2-c2ccccc2-c2ccccc2N(c2ccc(-c3ccc4c(c3)C3(c5ccccc5Oc5ccccc53)c3ccccc3-4)cc2)c2cccc(-c3cccc4c5ccccc5n(-c5ccccc5)c34)c2)cc1 ; CC1(c2ccccc2)c2ccccc2-c2ccc(-c3ccc(N(c4ccc(-n5c6ccccc6c6ccccc65)cc4)c4ccccc4-c4ccccc4-c4ccccc4-c4ccccc4)cc3)cc21]

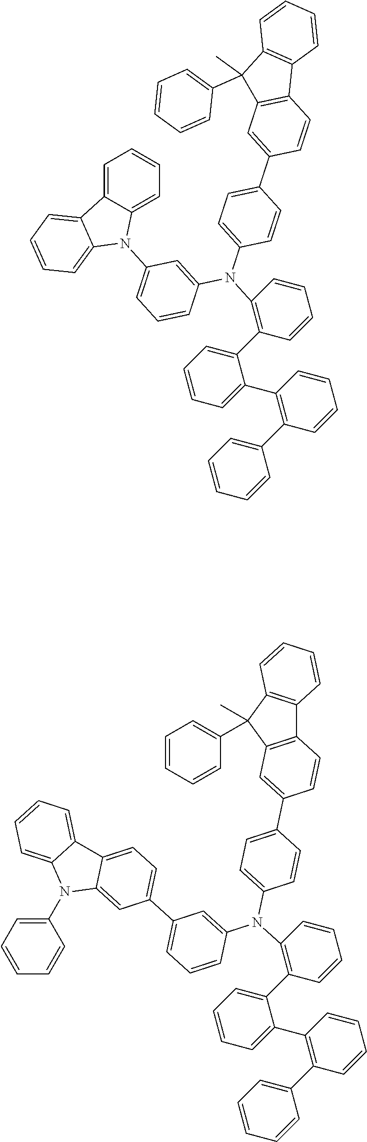 [SMILES: CC1(c2ccccc2)c2ccccc2-c2ccc(-c3ccc(N(c4cccc(-n5c6ccccc6c6ccccc65)c4)c4ccccc4-c4ccccc4-c4ccccc4-c4ccccc4)cc3)cc21 ; CC1(c2ccccc2)c2ccccc2-c2ccc(-c3ccc(N(c4cccc(-c5ccc6c7ccccc7n(-c7ccccc7)c6c5)c4)c4ccccc4-c4ccccc4-c4ccccc4-c4ccccc4)cc3)cc21]

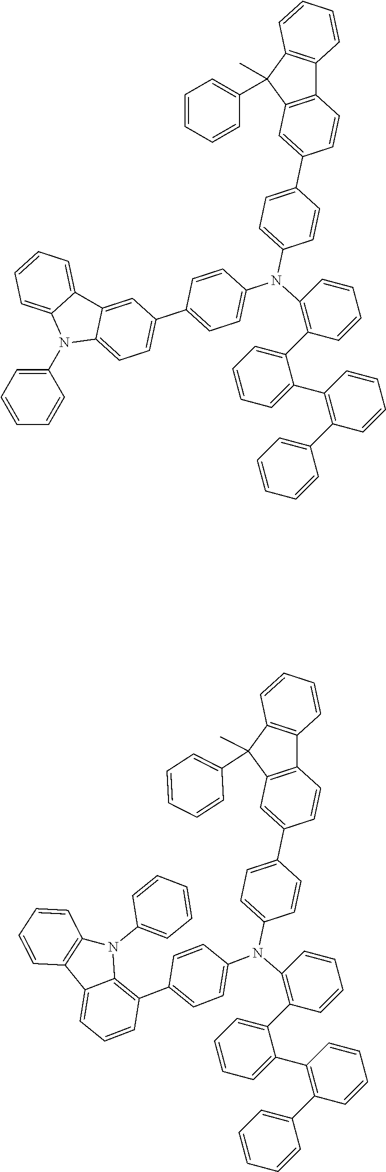 [SMILES: CC1(c2ccccc2)c2ccccc2-c2ccc(-c3ccc(N(c4ccc(-c5ccc6c(c5)c5ccccc5n6-c5ccccc5)cc4)c4ccccc4-c4ccccc4-c4ccccc4-c4ccccc4)cc3)cc21 ; CC1(c2ccccc2)c2ccccc2-c2ccc(-c3ccc(N(c4ccc(-c5cccc6c7ccccc7n(-c7ccccc7)c56)cc4)c4ccccc4-c4ccccc4-c4ccccc4-c4ccccc4)cc3)cc21]

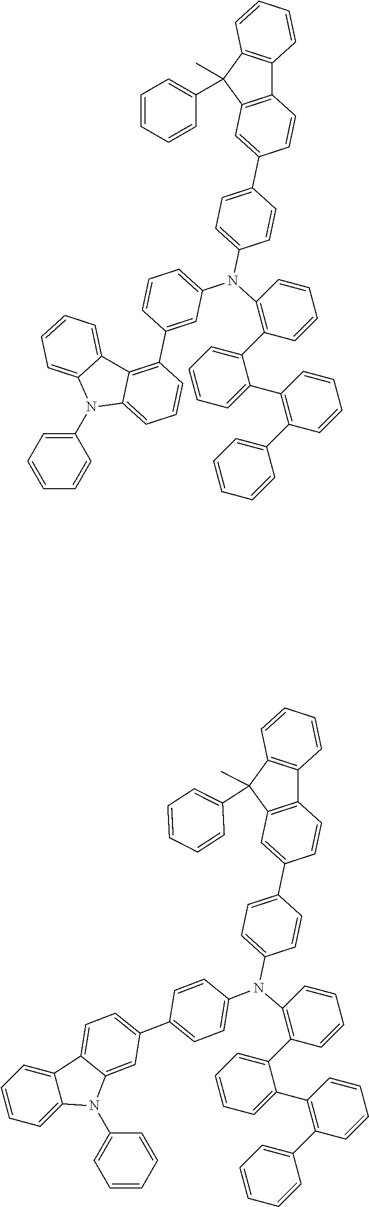 [SMILES: CC1(c2ccccc2)c2ccccc2-c2ccc(-c3ccc(N(c4cccc(-c5cccc6c5c5ccccc5n6-c5ccccc5)c4)c4ccccc4-c4ccccc4-c4ccccc4-c4ccccc4)cc3)cc21 ; CC1(c2ccccc2)c2ccccc2-c2ccc(-c3ccc(N(c4ccc(-c5ccc6c7ccccc7n(-c7ccccc7)c6c5)cc4)c4ccccc4-c4ccccc4-c4ccccc4-c4ccccc4)cc3)cc21]

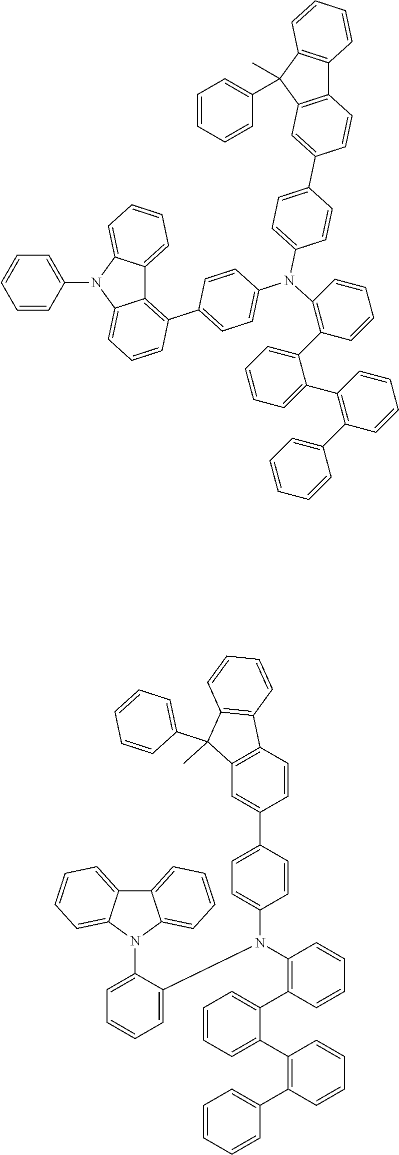 [SMILES: CC1(c2ccccc2)c2ccccc2-c2ccc(-c3ccc(N(c4ccc(-c5cccc6c5c5ccccc5n6-c5ccccc5)cc4)c4ccccc4-c4ccccc4-c4ccccc4-c4ccccc4)cc3)cc21 ; CC1(c2ccccc2)c2ccccc2-c2ccc(-c3ccc(N(c4ccccc4-c4ccccc4-c4ccccc4-c4ccccc4)c4ccccc4-n4c5ccccc5c5ccccc54)cc3)cc21]

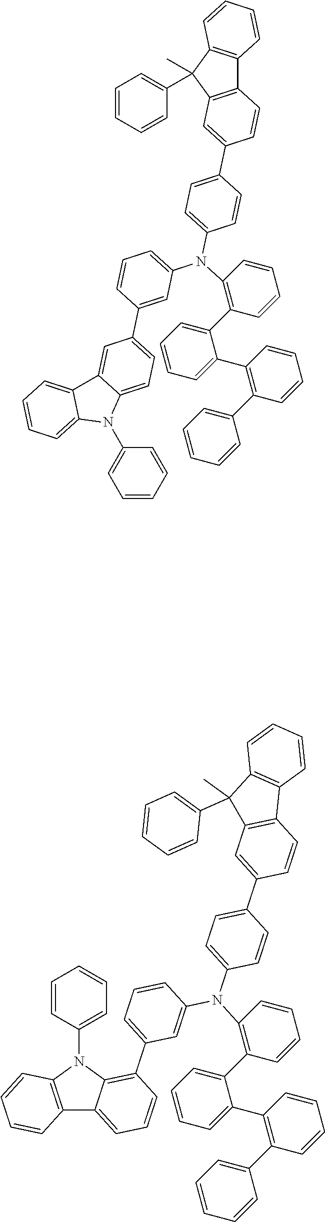 [SMILES: CC1(c2ccccc2)c2ccccc2-c2ccc(-c3ccc(N(c4cccc(-c5ccc6c(c5)c5ccccc5n6-c5ccccc5)c4)c4ccccc4-c4ccccc4-c4ccccc4-c4ccccc4)cc3)cc21 ; CC1(c2ccccc2)c2ccccc2-c2ccc(-c3ccc(N(c4cccc(-c5cccc6c7ccccc7n(-c7ccccc7)c56)c4)c4ccccc4-c4ccccc4-c4ccccc4-c4ccccc4)cc3)cc21]

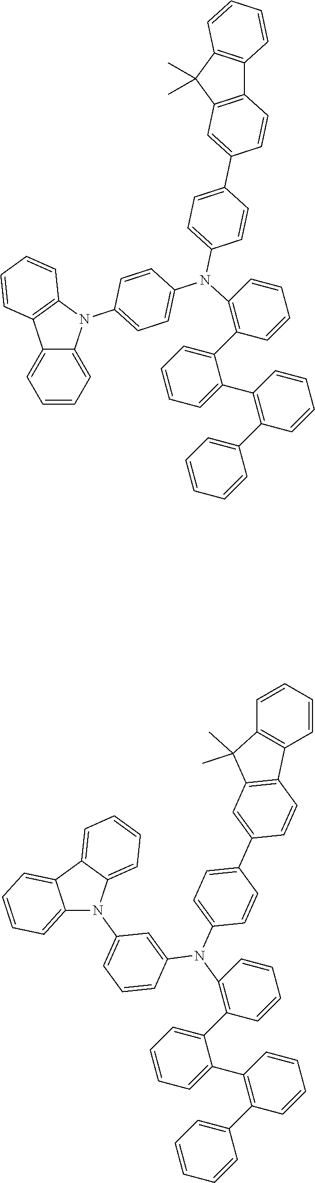 [SMILES: CC1(C)c2ccccc2-c2ccc(-c3ccc(N(c4ccc(-n5c6ccccc6c6ccccc65)cc4)c4ccccc4-c4ccccc4-c4ccccc4-c4ccccc4)cc3)cc21 ; CC1(C)c2ccccc2-c2ccc(-c3ccc(N(c4cccc(-n5c6ccccc6c6ccccc65)c4)c4ccccc4-c4ccccc4-c4ccccc4-c4ccccc4)cc3)cc21]

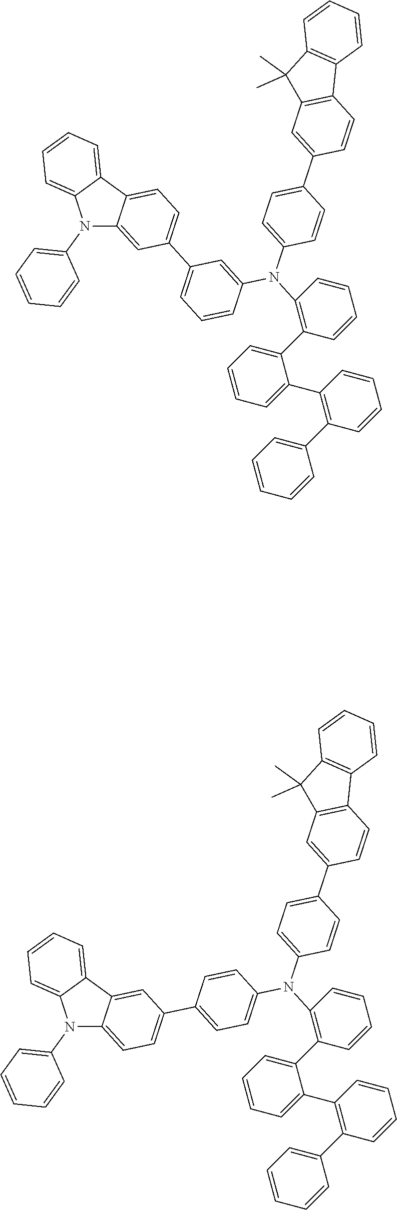 [SMILES: CC1(C)c2ccccc2-c2ccc(-c3ccc(N(c4cccc(-c5ccc6c7ccccc7n(-c7ccccc7)c6c5)c4)c4ccccc4-c4ccccc4-c4ccccc4-c4ccccc4)cc3)cc21 ; CC1(C)c2ccccc2-c2ccc(-c3ccc(N(c4ccc(-c5ccc6c(c5)c5ccccc5n6-c5ccccc5)cc4)c4ccccc4-c4ccccc4-c4ccccc4-c4ccccc4)cc3)cc21]

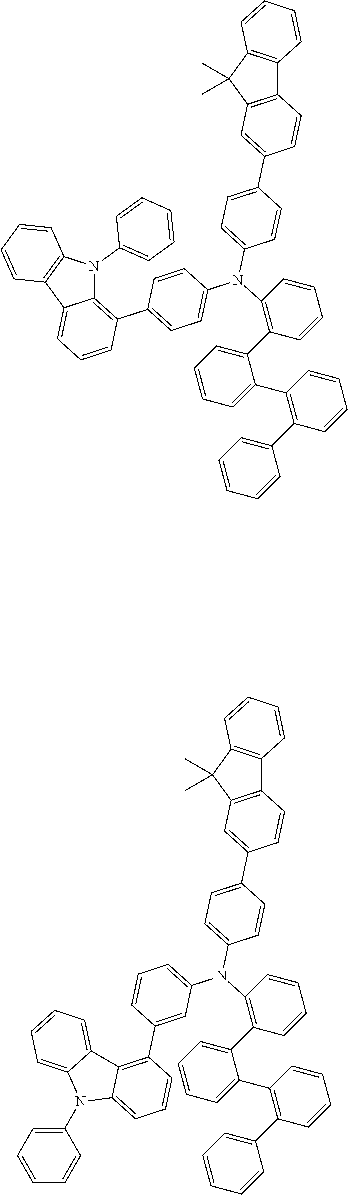 [SMILES: CC1(C)c2ccccc2-c2ccc(-c3ccc(N(c4ccc(-c5cccc6c7ccccc7n(-c7ccccc7)c56)cc4)c4ccccc4-c4ccccc4-c4ccccc4-c4ccccc4)cc3)cc21 ; CC1(C)c2ccccc2-c2ccc(-c3ccc(N(c4cccc(-c5cccc6c5c5ccccc5n6-c5ccccc5)c4)c4ccccc4-c4ccccc4-c4ccccc4-c4ccccc4)cc3)cc21]

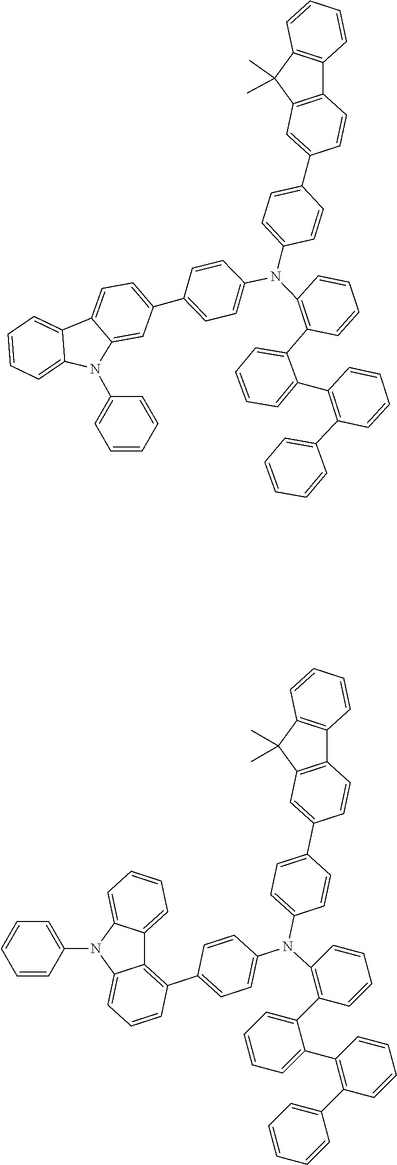 [SMILES: CC1(C)c2ccccc2-c2ccc(-c3ccc(N(c4ccc(-c5ccc6c7ccccc7n(-c7ccccc7)c6c5)cc4)c4ccccc4-c4ccccc4-c4ccccc4-c4ccccc4)cc3)cc21 ; CC1(C)c2ccccc2-c2ccc(-c3ccc(N(c4ccc(-c5cccc6c5c5ccccc5n6-c5ccccc5)cc4)c4ccccc4-c4ccccc4-c4ccccc4-c4ccccc4)cc3)cc21]

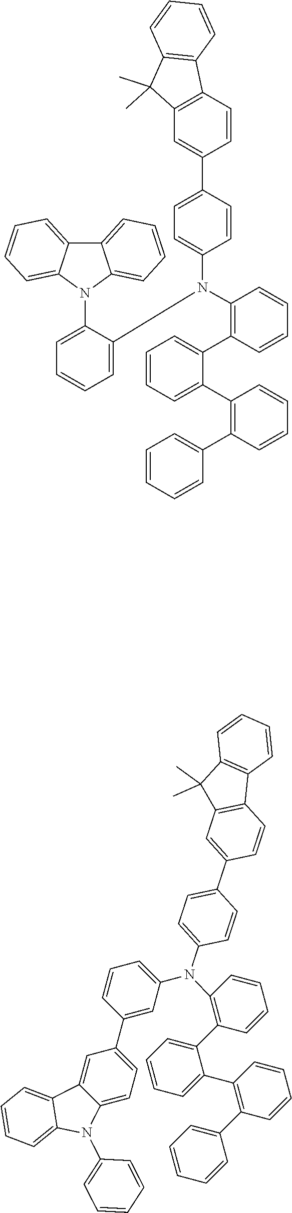 [SMILES: CC1(C)c2ccccc2-c2ccc(-c3ccc(N(c4ccccc4-c4ccccc4-c4ccccc4-c4ccccc4)c4ccccc4-n4c5ccccc5c5ccccc54)cc3)cc21 ; CC1(C)c2ccccc2-c2ccc(-c3ccc(N(c4cccc(-c5ccc6c(c5)c5ccccc5n6-c5ccccc5)c4)c4ccccc4-c4ccccc4-c4ccccc4-c4ccccc4)cc3)cc21]

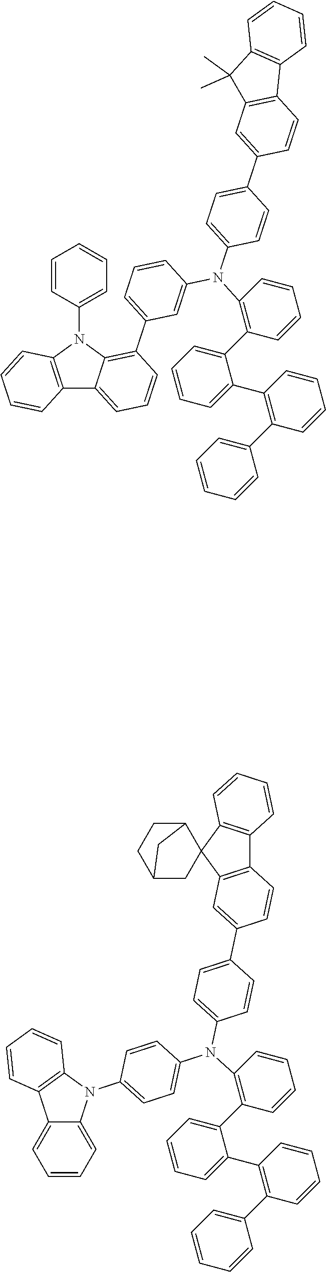 [SMILES: CC1(C)c2ccccc2-c2ccc(-c3ccc(N(c4cccc(-c5cccc6c7ccccc7n(-c7ccccc7)c56)c4)c4ccccc4-c4ccccc4-c4ccccc4-c4ccccc4)cc3)cc21 ; c1ccc(-c2ccccc2-c2ccccc2-c2ccccc2N(c2ccc(-c3ccc4c(c3)C3(CC5CCC3C5)c3ccccc3-4)cc2)c2ccc(-n3c4ccccc4c4ccccc43)cc2)cc1]

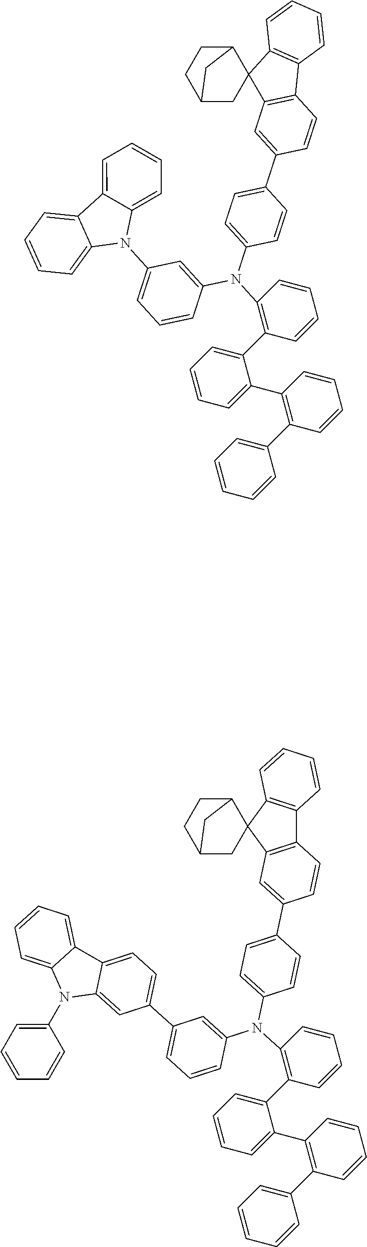 [SMILES: c1ccc(-c2ccccc2-c2ccccc2-c2ccccc2N(c2ccc(-c3ccc4c(c3)C3(CC5CCC3C5)c3ccccc3-4)cc2)c2cccc(-n3c4ccccc4c4ccccc43)c2)cc1 ; c1ccc(-c2ccccc2-c2ccccc2-c2ccccc2N(c2ccc(-c3ccc4c(c3)C3(CC5CCC3C5)c3ccccc3-4)cc2)c2cccc(-c3ccc4c5ccccc5n(-c5ccccc5)c4c3)c2)cc1]

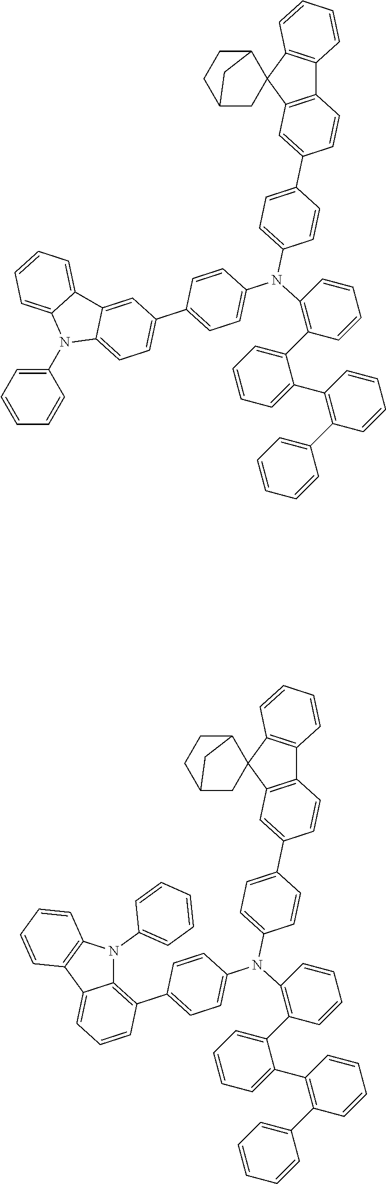 [SMILES: c1ccc(-c2ccccc2-c2ccccc2-c2ccccc2N(c2ccc(-c3ccc4c(c3)C3(CC5CCC3C5)c3ccccc3-4)cc2)c2ccc(-c3ccc4c(c3)c3ccccc3n4-c3ccccc3)cc2)cc1 ; c1ccc(-c2ccccc2-c2ccccc2-c2ccccc2N(c2ccc(-c3ccc4c(c3)C3(CC5CCC3C5)c3ccccc3-4)cc2)c2ccc(-c3cccc4c5ccccc5n(-c5ccccc5)c34)cc2)cc1]

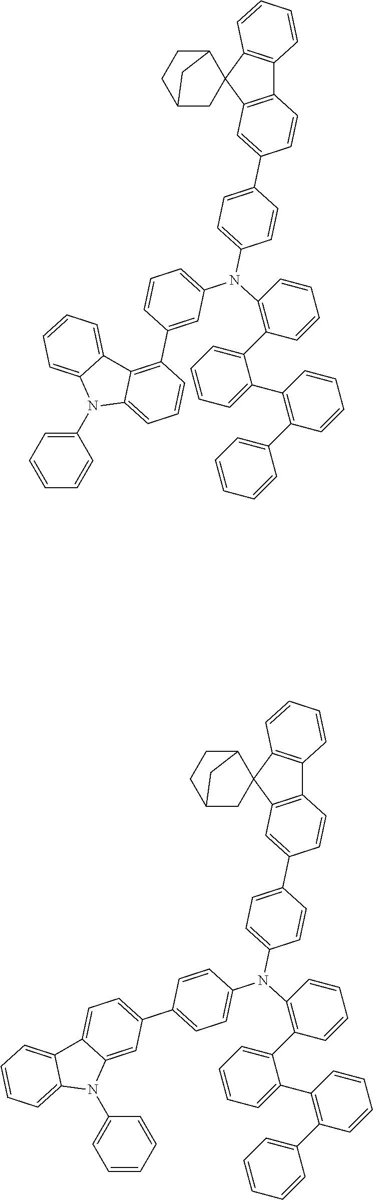 [SMILES: c1ccc(-c2ccccc2-c2ccccc2-c2ccccc2N(c2ccc(-c3ccc4c(c3)C3(CC5CCC3C5)c3ccccc3-4)cc2)c2cccc(-c3cccc4c3c3ccccc3n4-c3ccccc3)c2)cc1 ; c1ccc(-c2ccccc2-c2ccccc2-c2ccccc2N(c2ccc(-c3ccc4c(c3)C3(CC5CCC3C5)c3ccccc3-4)cc2)c2ccc(-c3ccc4c5ccccc5n(-c5ccccc5)c4c3)cc2)cc1]

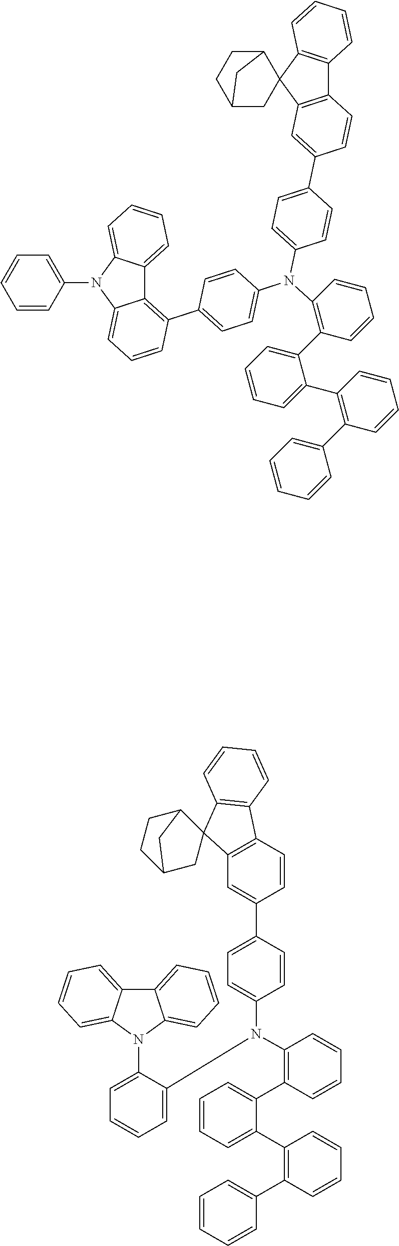 [SMILES: c1ccc(-c2ccccc2-c2ccccc2-c2ccccc2N(c2ccc(-c3ccc4c(c3)C3(CC5CCC3C5)c3ccccc3-4)cc2)c2ccc(-c3cccc4c3c3ccccc3n4-c3ccccc3)cc2)cc1 ; c1ccc(-c2ccccc2-c2ccccc2-c2ccccc2N(c2ccc(-c3ccc4c(c3)C3(CC5CCC3C5)c3ccccc3-4)cc2)c2ccccc2-n2c3ccccc3c3ccccc32)cc1]

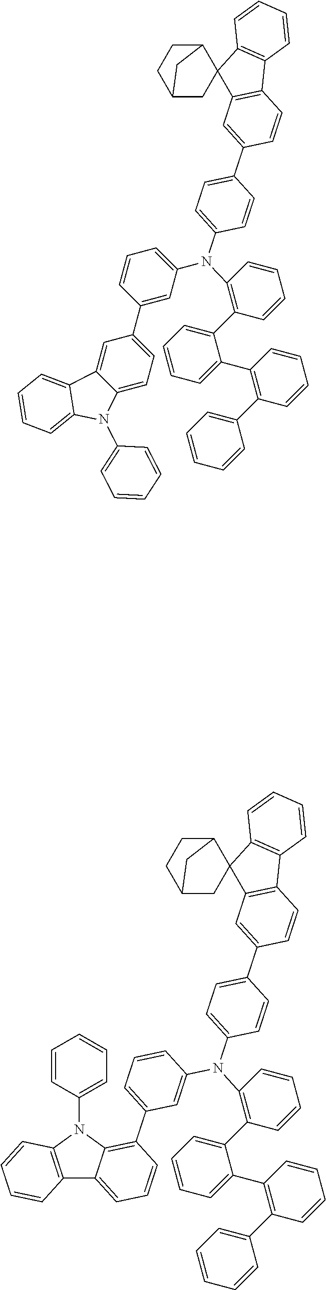 [SMILES: c1ccc(-c2ccccc2-c2ccccc2-c2ccccc2N(c2ccc(-c3ccc4c(c3)C3(CC5CCC3C5)c3ccccc3-4)cc2)c2cccc(-c3ccc4c(c3)c3ccccc3n4-c3ccccc3)c2)cc1 ; c1ccc(-c2ccccc2-c2ccccc2-c2ccccc2N(c2ccc(-c3ccc4c(c3)C3(CC5CCC3C5)c3ccccc3-4)cc2)c2cccc(-c3cccc4c5ccccc5n(-c5ccccc5)c34)c2)cc1]

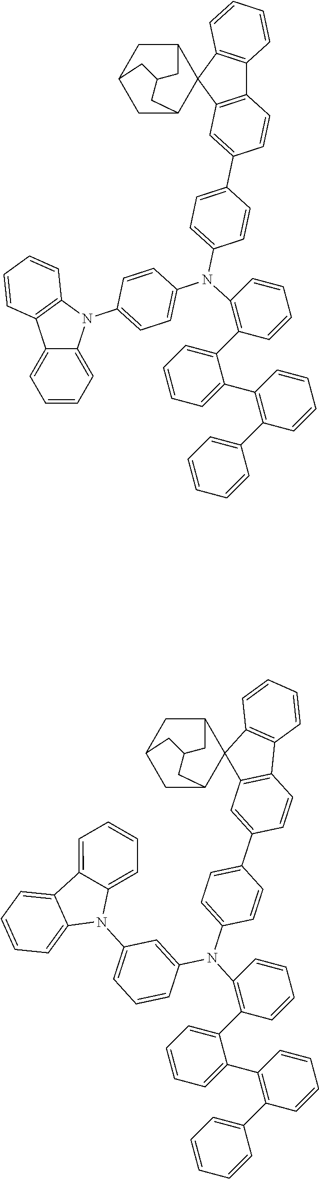 [SMILES: c1ccc(-c2ccccc2-c2ccccc2-c2ccccc2N(c2ccc(-c3ccc4c(c3)C3(c5ccccc5-4)C4CC5CC(C4)CC3C5)cc2)c2ccc(-n3c4ccccc4c4ccccc43)cc2)cc1 ; c1ccc(-c2ccccc2-c2ccccc2-c2ccccc2N(c2ccc(-c3ccc4c(c3)C3(c5ccccc5-4)C4CC5CC(C4)CC3C5)cc2)c2cccc(-n3c4ccccc4c4ccccc43)c2)cc1]

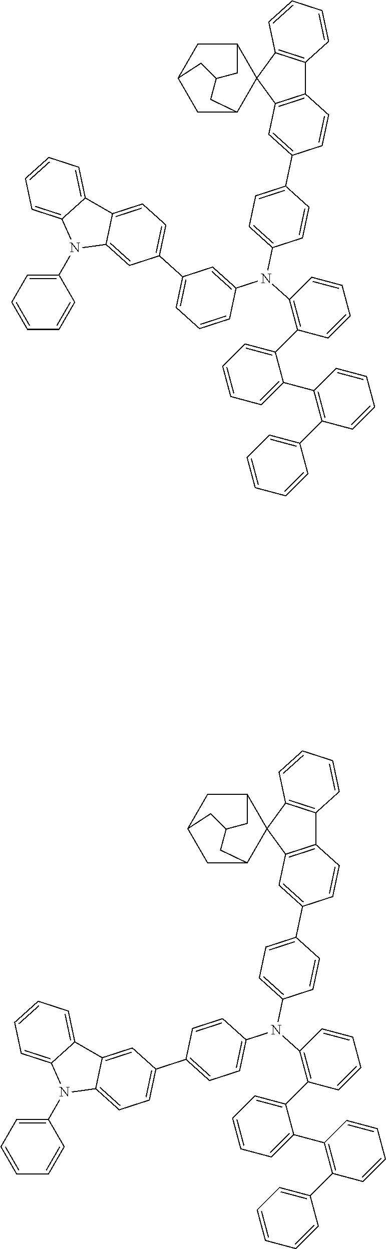 [SMILES: c1ccc(-c2ccccc2-c2ccccc2-c2ccccc2N(c2ccc(-c3ccc4c(c3)C3(c5ccccc5-4)C4CC5CC(C4)CC3C5)cc2)c2cccc(-c3ccc4c5ccccc5n(-c5ccccc5)c4c3)c2)cc1 ; c1ccc(-c2ccccc2-c2ccccc2-c2ccccc2N(c2ccc(-c3ccc4c(c3)C3(c5ccccc5-4)C4CC5CC(C4)CC3C5)cc2)c2ccc(-c3ccc4c(c3)c3ccccc3n4-c3ccccc3)cc2)cc1]

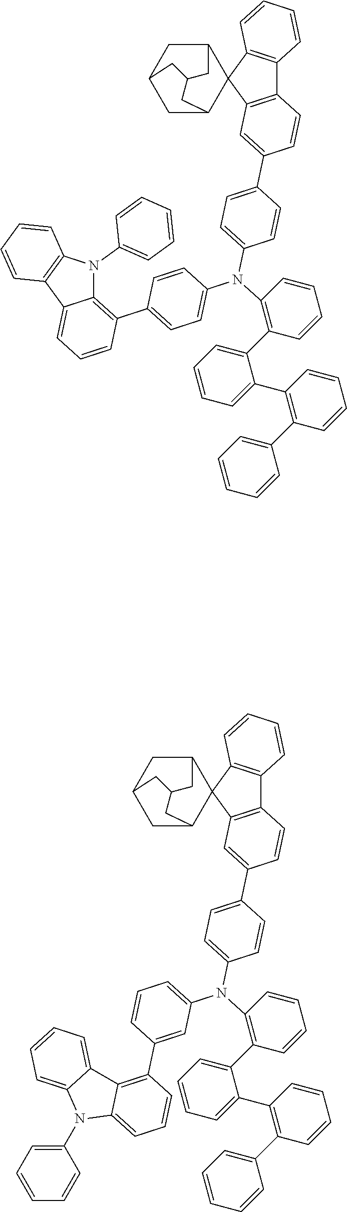 [SMILES: c1ccc(-c2ccccc2-c2ccccc2-c2ccccc2N(c2ccc(-c3ccc4c(c3)C3(c5ccccc5-4)C4CC5CC(C4)CC3C5)cc2)c2ccc(-c3cccc4c5ccccc5n(-c5ccccc5)c34)cc2)cc1 ; c1ccc(-c2ccccc2-c2ccccc2-c2ccccc2N(c2ccc(-c3ccc4c(c3)C3(c5ccccc5-4)C4CC5CC(C4)CC3C5)cc2)c2cccc(-c3cccc4c3c3ccccc3n4-c3ccccc3)c2)cc1]

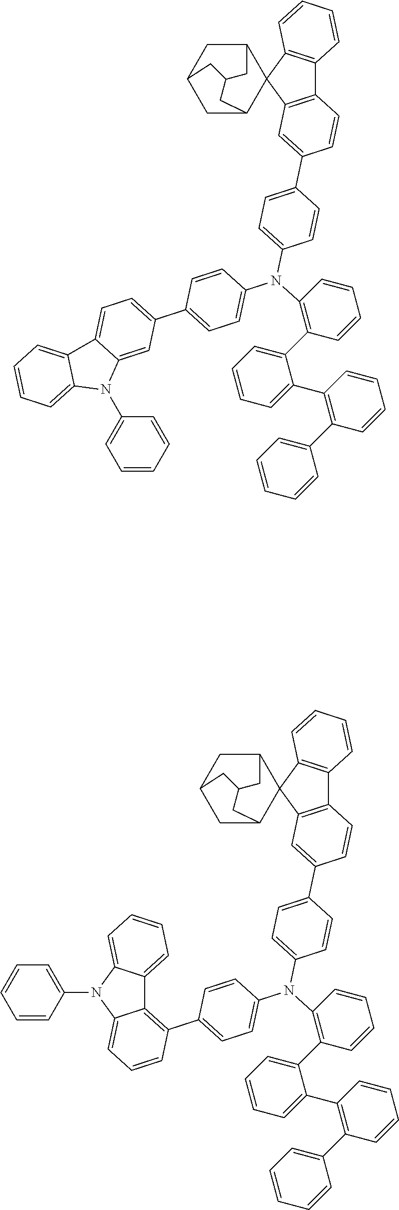 [SMILES: c1ccc(-c2ccccc2-c2ccccc2-c2ccccc2N(c2ccc(-c3ccc4c(c3)C3(c5ccccc5-4)C4CC5CC(C4)CC3C5)cc2)c2ccc(-c3ccc4c5ccccc5n(-c5ccccc5)c4c3)cc2)cc1 ; c1ccc(-c2ccccc2-c2ccccc2-c2ccccc2N(c2ccc(-c3ccc4c(c3)C3(c5ccccc5-4)C4CC5CC(C4)CC3C5)cc2)c2ccc(-c3cccc4c3c3ccccc3n4-c3ccccc3)cc2)cc1]

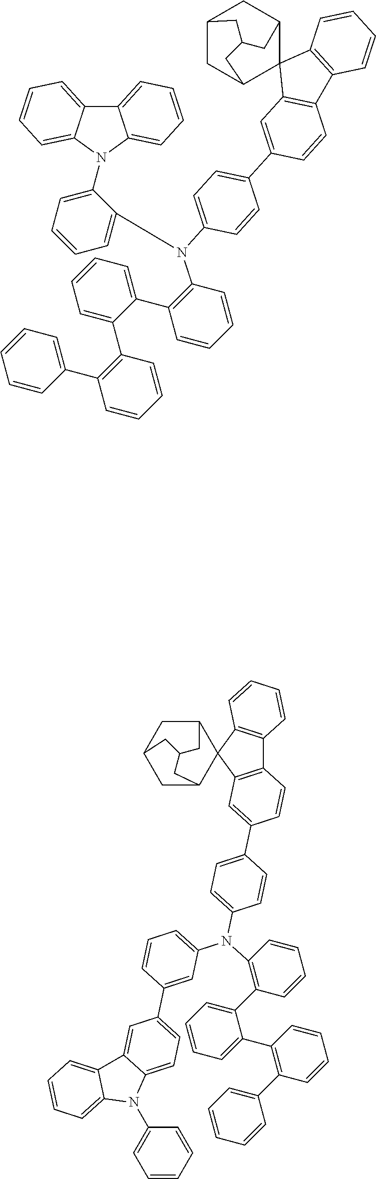 [SMILES: c1ccc(-c2ccccc2-c2ccccc2-c2ccccc2N(c2ccc(-c3ccc4c(c3)C3(c5ccccc5-4)C4CC5CC(C4)CC3C5)cc2)c2ccccc2-n2c3ccccc3c3ccccc32)cc1 ; c1ccc(-c2ccccc2-c2ccccc2-c2ccccc2N(c2ccc(-c3ccc4c(c3)C3(c5ccccc5-4)C4CC5CC(C4)CC3C5)cc2)c2cccc(-c3ccc4c(c3)c3ccccc3n4-c3ccccc3)c2)cc1]

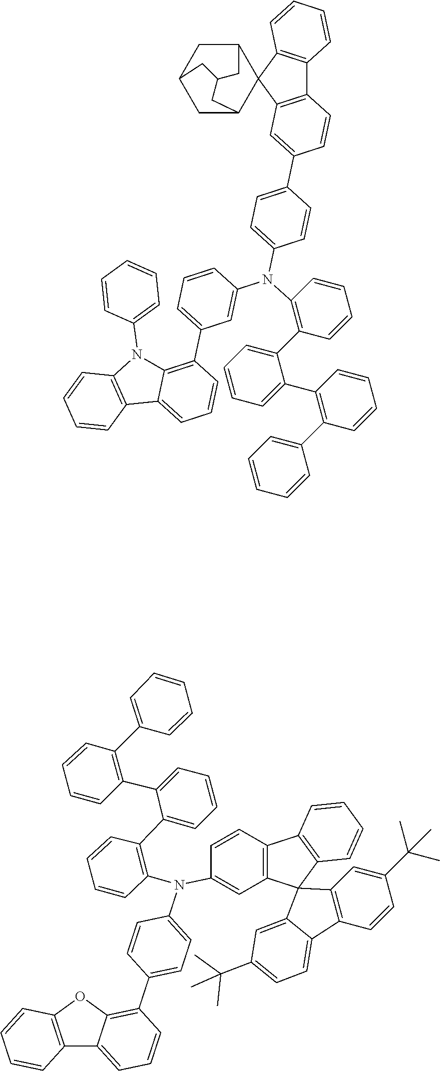 [SMILES: c1ccc(-c2ccccc2-c2ccccc2-c2ccccc2N(c2ccc(-c3ccc4c(c3)C3(c5ccccc5-4)C4CC5CC(C4)CC3C5)cc2)c2cccc(-c3cccc4c5ccccc5n(-c5ccccc5)c34)c2)cc1 ; CC(C)(C)c1ccc2c(c1)C1(c3ccccc3-c3ccc(N(c4ccc(-c5cccc6c5oc5ccccc56)cc4)c4ccccc4-c4ccccc4-c4ccccc4-c4ccccc4)cc31)c1cc(C(C)(C)C)ccc1-2]

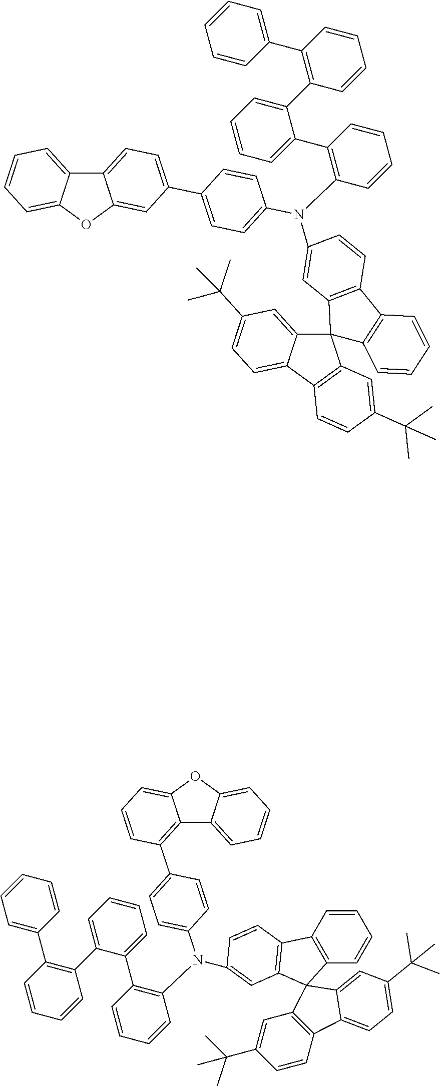 [SMILES: CC(C)(C)c1ccc2c(c1)C1(c3ccccc3-c3ccc(N(c4ccc(-c5ccc6c(c5)oc5ccccc56)cc4)c4ccccc4-c4ccccc4-c4ccccc4-c4ccccc4)cc31)c1cc(C(C)(C)C)ccc1-2 ; CC(C)(C)c1ccc2c(c1)C1(c3ccccc3-c3ccc(N(c4ccc(-c5cccc6oc7ccccc7c56)cc4)c4ccccc4-c4ccccc4-c4ccccc4-c4ccccc4)cc31)c1cc(C(C)(C)C)ccc1-2]

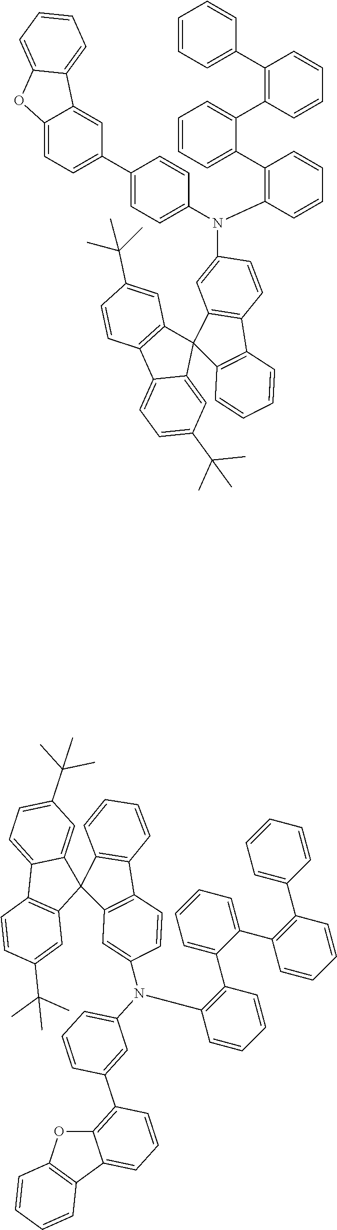 [SMILES: CC(C)(C)c1ccc2c(c1)C1(c3ccccc3-c3ccc(N(c4ccc(-c5ccc6oc7ccccc7c6c5)cc4)c4ccccc4-c4ccccc4-c4ccccc4-c4ccccc4)cc31)c1cc(C(C)(C)C)ccc1-2 ; CC(C)(C)c1ccc2c(c1)C1(c3ccccc3-c3ccc(N(c4cccc(-c5cccc6c5oc5ccccc56)c4)c4ccccc4-c4ccccc4-c4ccccc4-c4ccccc4)cc31)c1cc(C(C)(C)C)ccc1-2]

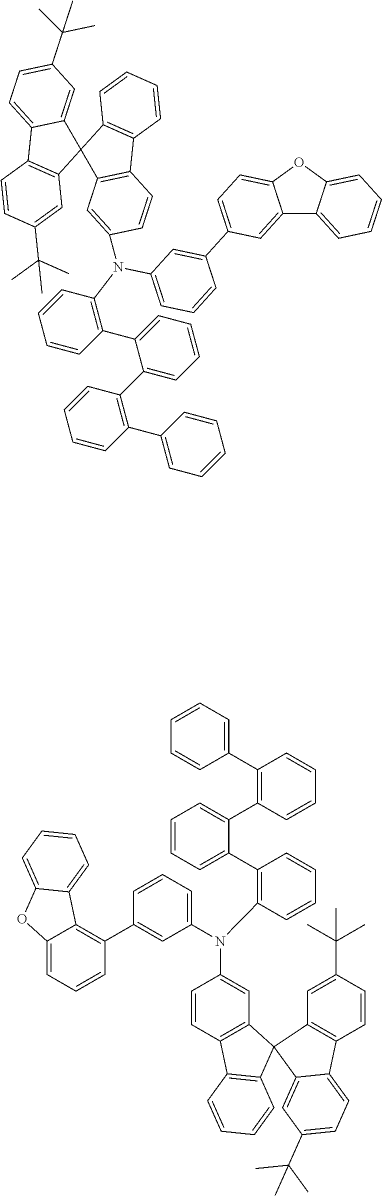 [SMILES: CC(C)(C)c1ccc2c(c1)C1(c3ccccc3-c3ccc(N(c4cccc(-c5ccc6oc7ccccc7c6c5)c4)c4ccccc4-c4ccccc4-c4ccccc4-c4ccccc4)cc31)c1cc(C(C)(C)C)ccc1-2 ; CC(C)(C)c1ccc2c(c1)C1(c3ccccc3-c3ccc(N(c4cccc(-c5cccc6oc7ccccc7c56)c4)c4ccccc4-c4ccccc4-c4ccccc4-c4ccccc4)cc31)c1cc(C(C)(C)C)ccc1-2]

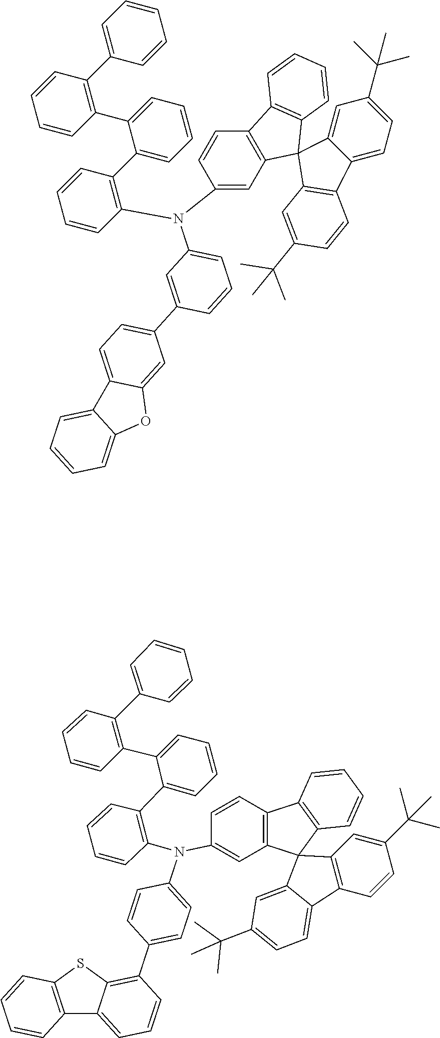 [SMILES: CC(C)(C)c1ccc2c(c1)C1(c3ccccc3-c3ccc(N(c4cccc(-c5ccc6c(c5)oc5ccccc56)c4)c4ccccc4-c4ccccc4-c4ccccc4-c4ccccc4)cc31)c1cc(C(C)(C)C)ccc1-2 ; CC(C)(C)c1ccc2c(c1)C1(c3ccccc3-c3ccc(N(c4ccc(-c5cccc6c5sc5ccccc56)cc4)c4ccccc4-c4ccccc4-c4ccccc4-c4ccccc4)cc31)c1cc(C(C)(C)C)ccc1-2]

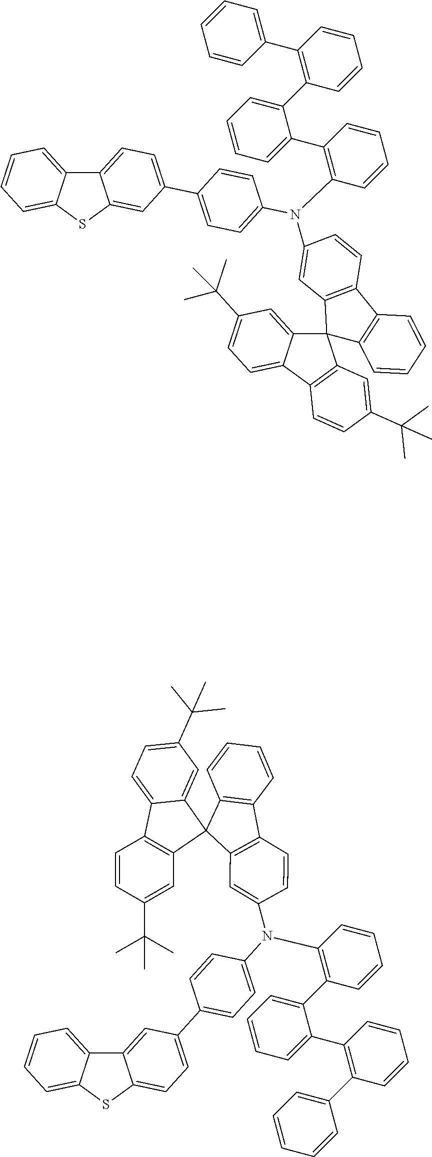 [SMILES: CC(C)(C)c1ccc2c(c1)C1(c3ccccc3-c3ccc(N(c4ccc(-c5ccc6c(c5)sc5ccccc56)cc4)c4ccccc4-c4ccccc4-c4ccccc4-c4ccccc4)cc31)c1cc(C(C)(C)C)ccc1-2 ; CC(C)(C)c1ccc2c(c1)C1(c3ccccc3-c3ccc(N(c4ccc(-c5ccc6sc7ccccc7c6c5)cc4)c4ccccc4-c4ccccc4-c4ccccc4-c4ccccc4)cc31)c1cc(C(C)(C)C)ccc1-2]

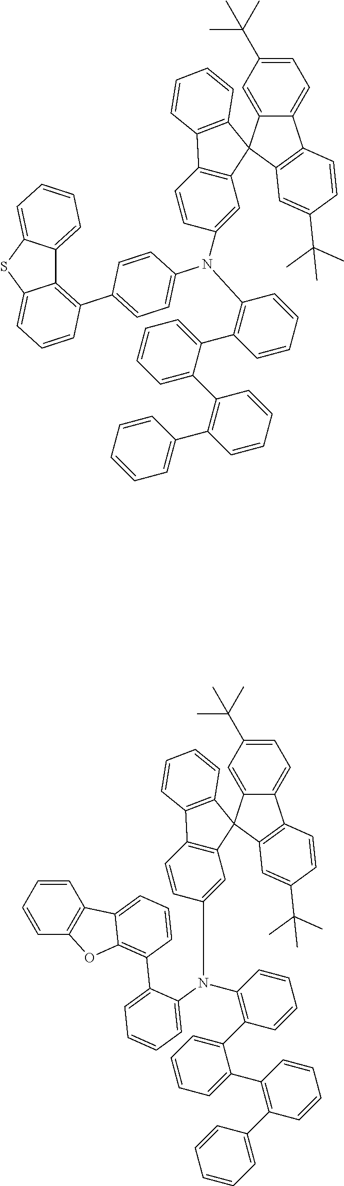 [SMILES: CC(C)(C)c1ccc2c(c1)C1(c3ccccc3-c3ccc(N(c4ccc(-c5cccc6sc7ccccc7c56)cc4)c4ccccc4-c4ccccc4-c4ccccc4-c4ccccc4)cc31)c1cc(C(C)(C)C)ccc1-2 ; CC(C)(C)c1ccc2c(c1)C1(c3ccccc3-c3ccc(N(c4ccccc4-c4ccccc4-c4ccccc4-c4ccccc4)c4ccccc4-c4cccc5c4oc4ccccc45)cc31)c1cc(C(C)(C)C)ccc1-2]

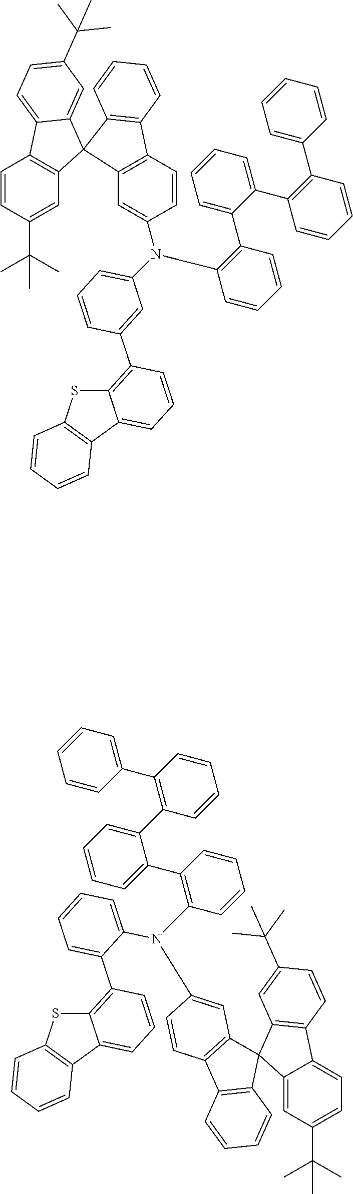 [SMILES: CC(C)(C)c1ccc2c(c1)C1(c3ccccc3-c3ccc(N(c4cccc(-c5cccc6c5sc5ccccc56)c4)c4ccccc4-c4ccccc4-c4ccccc4-c4ccccc4)cc31)c1cc(C(C)(C)C)ccc1-2 ; CC(C)(C)c1ccc2c(c1)C1(c3ccccc3-c3ccc(N(c4ccccc4-c4ccccc4-c4ccccc4-c4ccccc4)c4ccccc4-c4cccc5c4sc4ccccc45)cc31)c1cc(C(C)(C)C)ccc1-2]

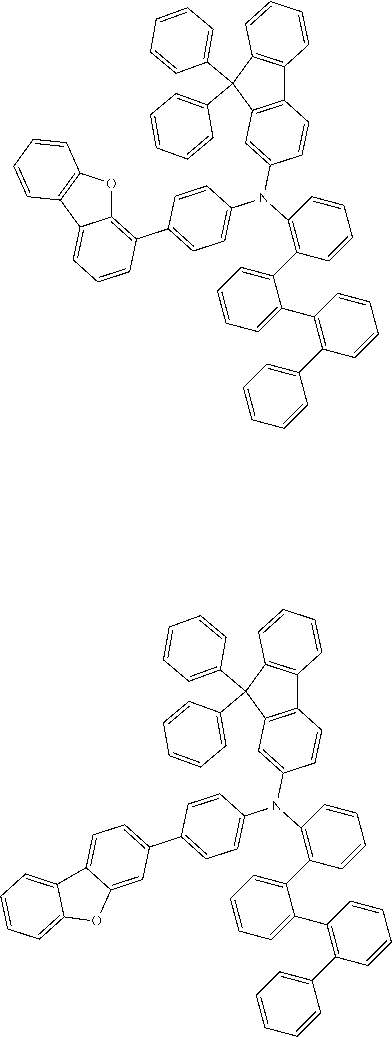 [SMILES: c1ccc(-c2ccccc2-c2ccccc2-c2ccccc2N(c2ccc(-c3cccc4c3oc3ccccc34)cc2)c2ccc3c(c2)C(c2ccccc2)(c2ccccc2)c2ccccc2-3)cc1 ; c1ccc(-c2ccccc2-c2ccccc2-c2ccccc2N(c2ccc(-c3ccc4c(c3)oc3ccccc34)cc2)c2ccc3c(c2)C(c2ccccc2)(c2ccccc2)c2ccccc2-3)cc1]

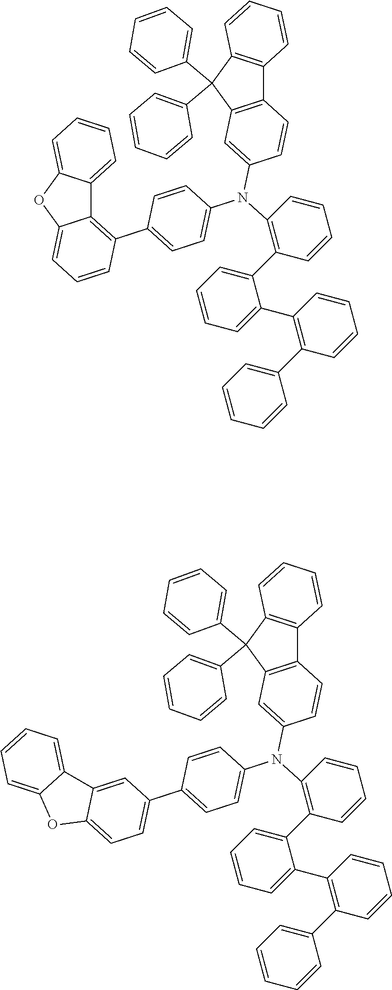 [SMILES: c1ccc(-c2ccccc2-c2ccccc2-c2ccccc2N(c2ccc(-c3cccc4oc5ccccc5c34)cc2)c2ccc3c(c2)C(c2ccccc2)(c2ccccc2)c2ccccc2-3)cc1 ; c1ccc(-c2ccccc2-c2ccccc2-c2ccccc2N(c2ccc(-c3ccc4oc5ccccc5c4c3)cc2)c2ccc3c(c2)C(c2ccccc2)(c2ccccc2)c2ccccc2-3)cc1]

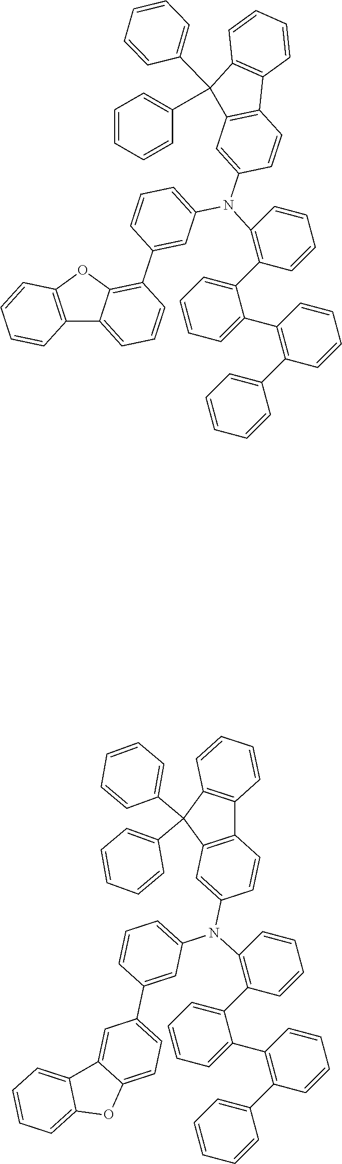 [SMILES: c1ccc(-c2ccccc2-c2ccccc2-c2ccccc2N(c2cccc(-c3cccc4c3oc3ccccc34)c2)c2ccc3c(c2)C(c2ccccc2)(c2ccccc2)c2ccccc2-3)cc1 ; c1ccc(-c2ccccc2-c2ccccc2-c2ccccc2N(c2cccc(-c3ccc4oc5ccccc5c4c3)c2)c2ccc3c(c2)C(c2ccccc2)(c2ccccc2)c2ccccc2-3)cc1]

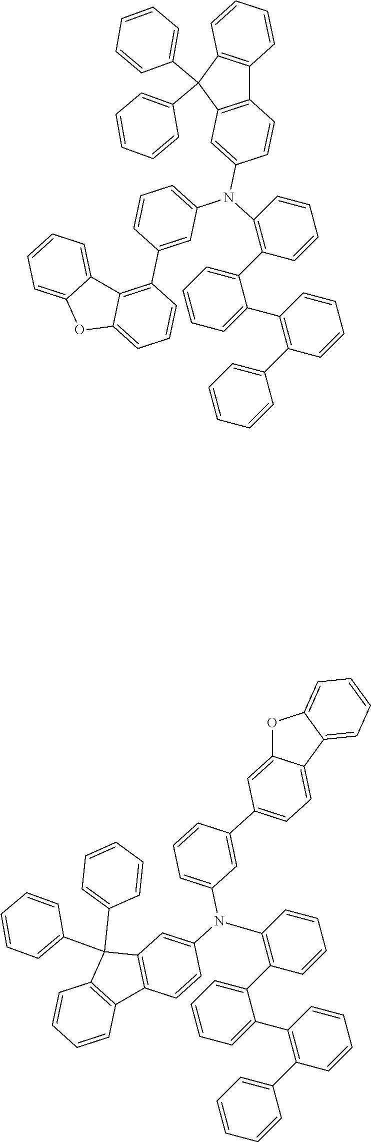 [SMILES: c1ccc(-c2ccccc2-c2ccccc2-c2ccccc2N(c2cccc(-c3cccc4oc5ccccc5c34)c2)c2ccc3c(c2)C(c2ccccc2)(c2ccccc2)c2ccccc2-3)cc1 ; c1ccc(-c2ccccc2-c2ccccc2-c2ccccc2N(c2cccc(-c3ccc4c(c3)oc3ccccc34)c2)c2ccc3c(c2)C(c2ccccc2)(c2ccccc2)c2ccccc2-3)cc1]

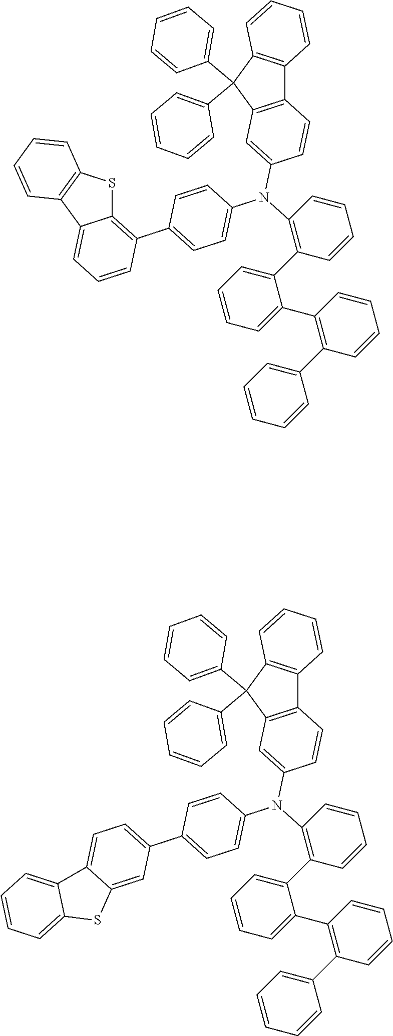 [SMILES: c1ccc(-c2ccccc2-c2ccccc2-c2ccccc2N(c2ccc(-c3cccc4c3sc3ccccc34)cc2)c2ccc3c(c2)C(c2ccccc2)(c2ccccc2)c2ccccc2-3)cc1 ; c1ccc(-c2ccccc2-c2ccccc2-c2ccccc2N(c2ccc(-c3ccc4c(c3)sc3ccccc34)cc2)c2ccc3c(c2)C(c2ccccc2)(c2ccccc2)c2ccccc2-3)cc1]

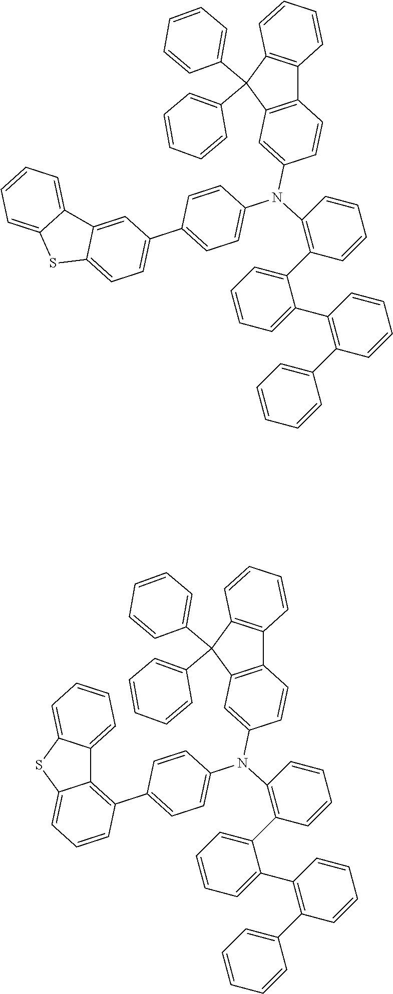 [SMILES: c1ccc(-c2ccccc2-c2ccccc2-c2ccccc2N(c2ccc(-c3ccc4sc5ccccc5c4c3)cc2)c2ccc3c(c2)C(c2ccccc2)(c2ccccc2)c2ccccc2-3)cc1 ; c1ccc(-c2ccccc2-c2ccccc2-c2ccccc2N(c2ccc(-c3cccc4sc5ccccc5c34)cc2)c2ccc3c(c2)C(c2ccccc2)(c2ccccc2)c2ccccc2-3)cc1]

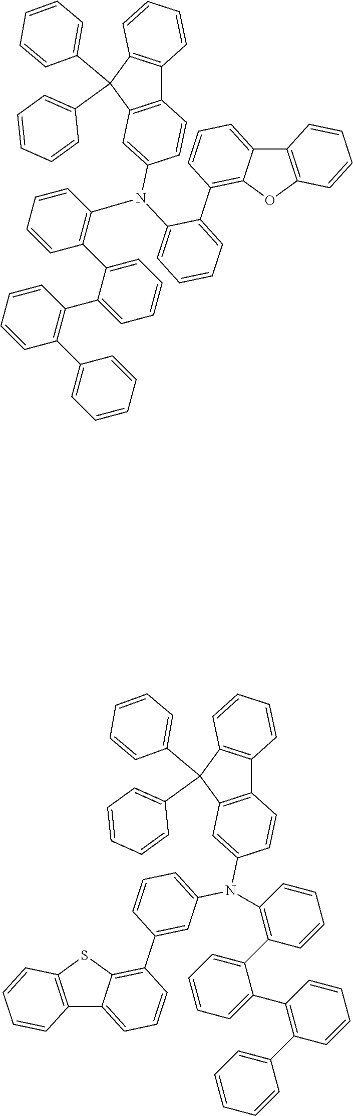 [SMILES: c1ccc(-c2ccccc2-c2ccccc2-c2ccccc2N(c2ccc3c(c2)C(c2ccccc2)(c2ccccc2)c2ccccc2-3)c2ccccc2-c2cccc3c2oc2ccccc23)cc1 ; c1ccc(-c2ccccc2-c2ccccc2-c2ccccc2N(c2cccc(-c3cccc4c3sc3ccccc34)c2)c2ccc3c(c2)C(c2ccccc2)(c2ccccc2)c2ccccc2-3)cc1]

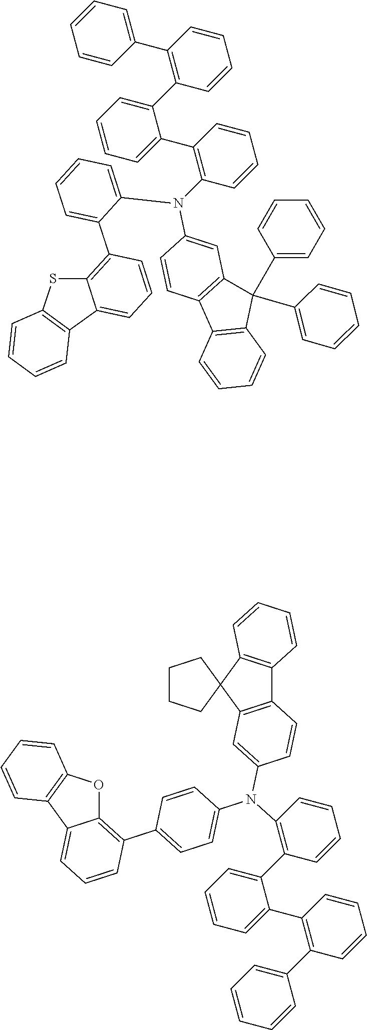 [SMILES: c1ccc(-c2ccccc2-c2ccccc2-c2ccccc2N(c2ccc3c(c2)C(c2ccccc2)(c2ccccc2)c2ccccc2-3)c2ccccc2-c2cccc3c2sc2ccccc23)cc1 ; c1ccc(-c2ccccc2-c2ccccc2-c2ccccc2N(c2ccc(-c3cccc4c3oc3ccccc34)cc2)c2ccc3c(c2)C2(CCCC2)c2ccccc2-3)cc1]

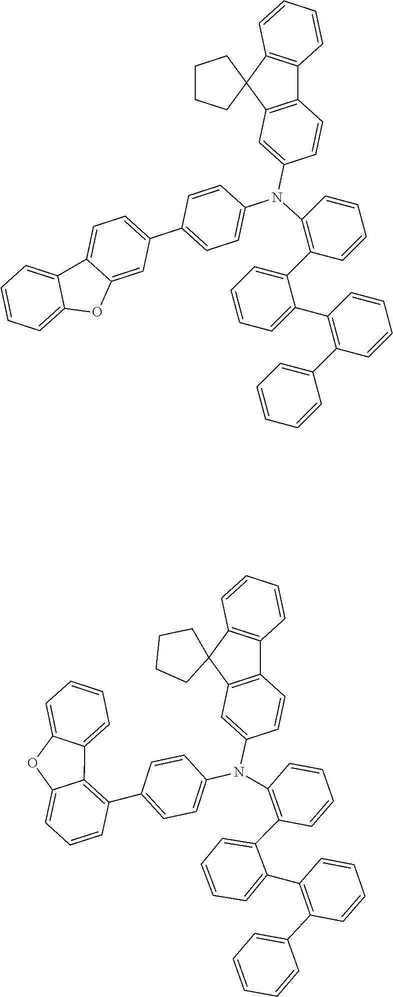 [SMILES: c1ccc(-c2ccccc2-c2ccccc2-c2ccccc2N(c2ccc(-c3ccc4c(c3)oc3ccccc34)cc2)c2ccc3c(c2)C2(CCCC2)c2ccccc2-3)cc1 ; c1ccc(-c2ccccc2-c2ccccc2-c2ccccc2N(c2ccc(-c3cccc4oc5ccccc5c34)cc2)c2ccc3c(c2)C2(CCCC2)c2ccccc2-3)cc1]

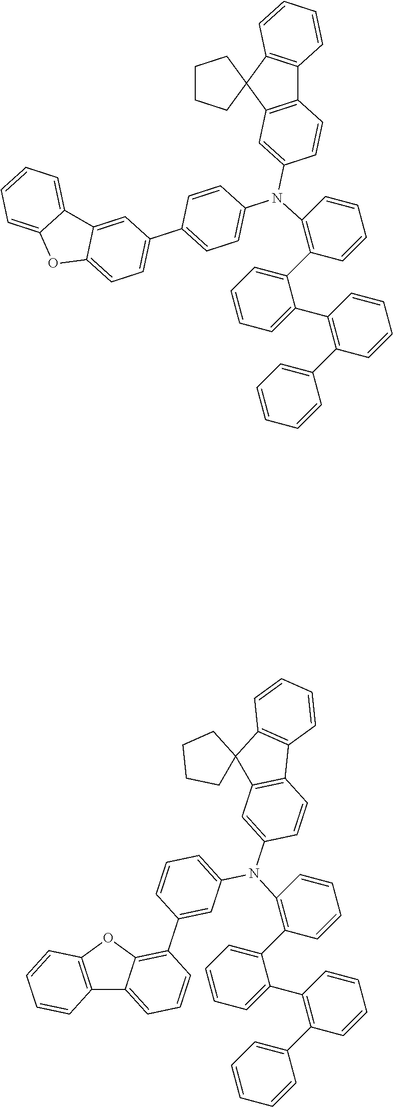 [SMILES: c1ccc(-c2ccccc2-c2ccccc2-c2ccccc2N(c2ccc(-c3ccc4oc5ccccc5c4c3)cc2)c2ccc3c(c2)C2(CCCC2)c2ccccc2-3)cc1 ; c1ccc(-c2ccccc2-c2ccccc2-c2ccccc2N(c2cccc(-c3cccc4c3oc3ccccc34)c2)c2ccc3c(c2)C2(CCCC2)c2ccccc2-3)cc1]

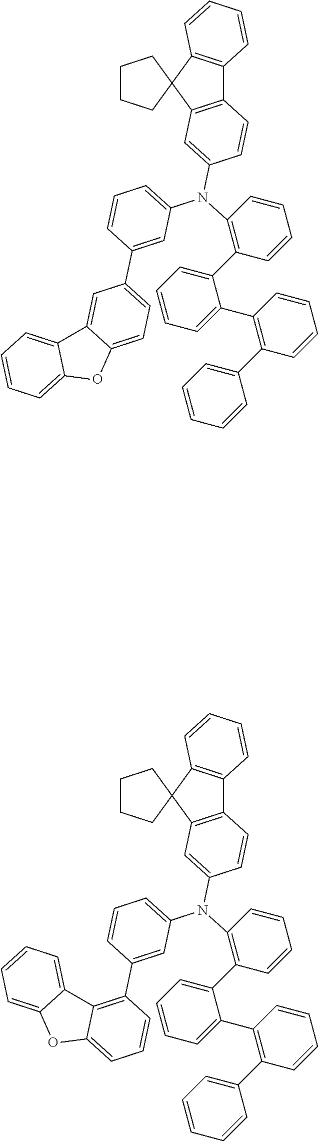 [SMILES: c1ccc(-c2ccccc2-c2ccccc2-c2ccccc2N(c2cccc(-c3ccc4oc5ccccc5c4c3)c2)c2ccc3c(c2)C2(CCCC2)c2ccccc2-3)cc1 ; c1ccc(-c2ccccc2-c2ccccc2-c2ccccc2N(c2cccc(-c3cccc4oc5ccccc5c34)c2)c2ccc3c(c2)C2(CCCC2)c2ccccc2-3)cc1]

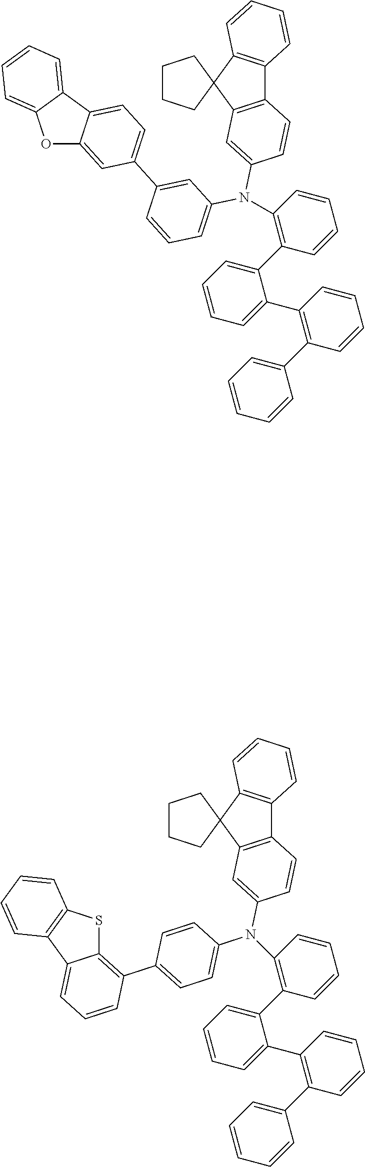 [SMILES: c1ccc(-c2ccccc2-c2ccccc2-c2ccccc2N(c2cccc(-c3ccc4c(c3)oc3ccccc34)c2)c2ccc3c(c2)C2(CCCC2)c2ccccc2-3)cc1 ; c1ccc(-c2ccccc2-c2ccccc2-c2ccccc2N(c2ccc(-c3cccc4c3sc3ccccc34)cc2)c2ccc3c(c2)C2(CCCC2)c2ccccc2-3)cc1]

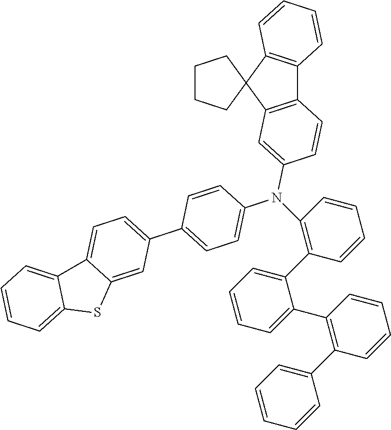 [SMILES: c1ccc(-c2ccccc2-c2ccccc2-c2ccccc2N(c2ccc(-c3ccc4c(c3)sc3ccccc34)cc2)c2ccc3c(c2)C2(CCCC2)c2ccccc2-3)cc1]

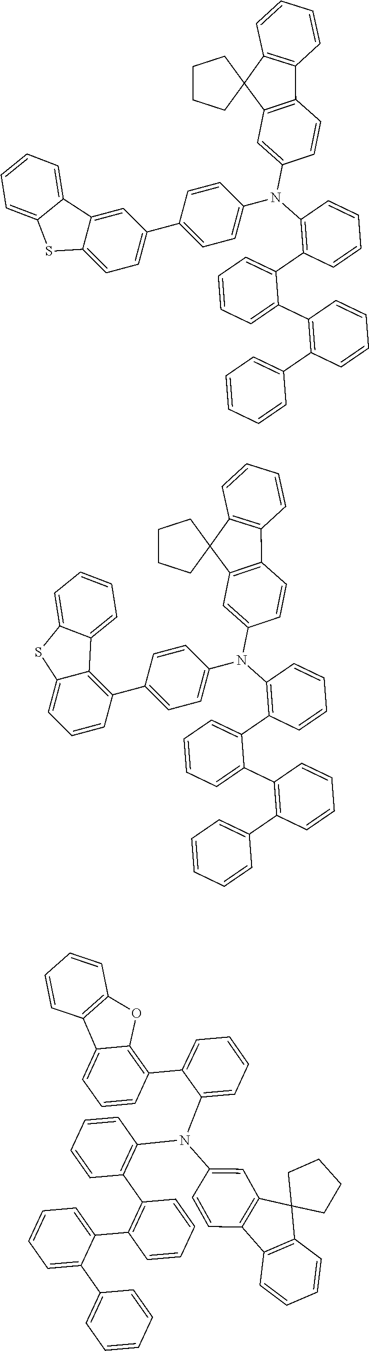

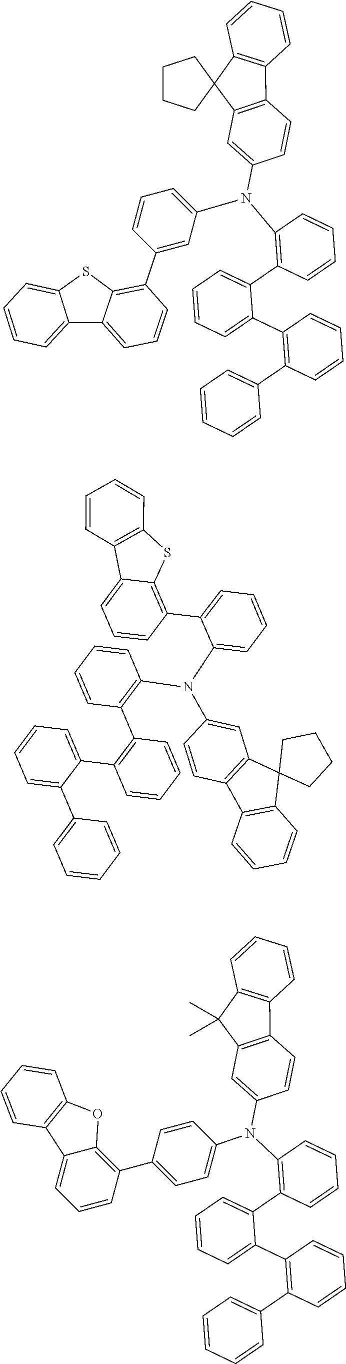 [SMILES: c1ccc(-c2ccccc2-c2ccccc2-c2ccccc2N(c2cccc(-c3cccc4c3sc3ccccc34)c2)c2ccc3c(c2)C2(CCCC2)c2ccccc2-3)cc1 ; c1ccc(-c2ccccc2-c2ccccc2-c2ccccc2N(c2ccc3c(c2)C2(CCCC2)c2ccccc2-3)c2ccccc2-c2cccc3c2sc2ccccc23)cc1 ; CC1(C)c2ccccc2-c2ccc(N(c3ccc(-c4cccc5c4oc4ccccc45)cc3)c3ccccc3-c3ccccc3-c3ccccc3-c3ccccc3)cc21]

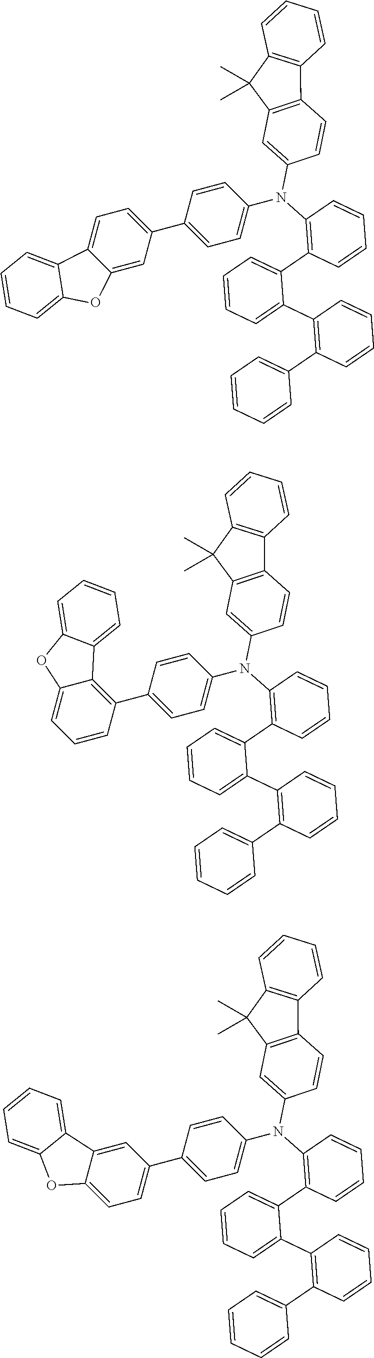 [SMILES: CC1(C)c2ccccc2-c2ccc(N(c3ccc(-c4ccc5c(c4)oc4ccccc45)cc3)c3ccccc3-c3ccccc3-c3ccccc3-c3ccccc3)cc21 ; CC1(C)c2ccccc2-c2ccc(N(c3ccc(-c4cccc5oc6ccccc6c45)cc3)c3ccccc3-c3ccccc3-c3ccccc3-c3ccccc3)cc21 ; CC1(C)c2ccccc2-c2ccc(N(c3ccc(-c4ccc5oc6ccccc6c5c4)cc3)c3ccccc3-c3ccccc3-c3ccccc3-c3ccccc3)cc21]

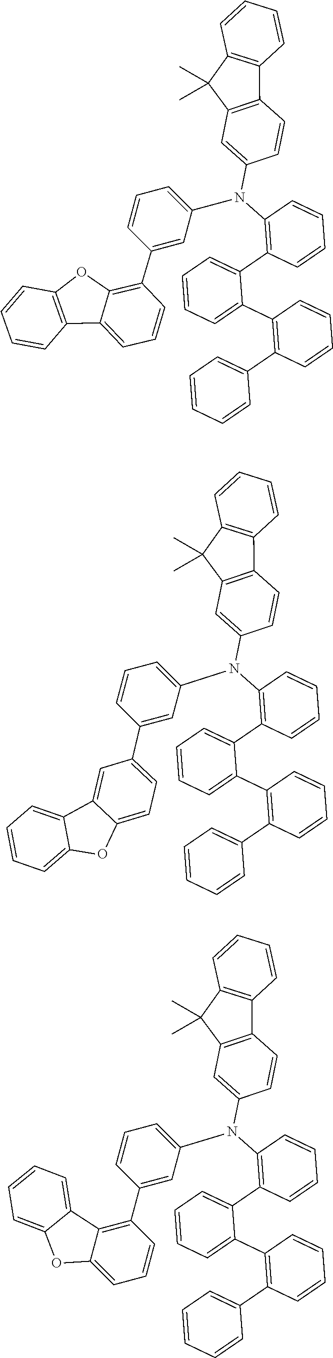

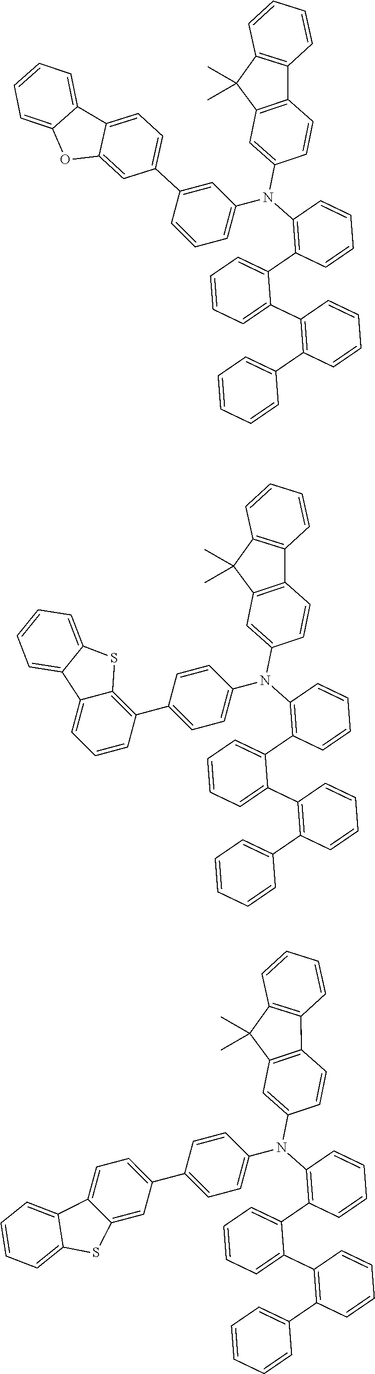 [SMILES: CC1(C)c2ccccc2-c2ccc(N(c3cccc(-c4ccc5c(c4)oc4ccccc45)c3)c3ccccc3-c3ccccc3-c3ccccc3-c3ccccc3)cc21 ; CC1(C)c2ccccc2-c2ccc(N(c3ccc(-c4cccc5c4sc4ccccc45)cc3)c3ccccc3-c3ccccc3-c3ccccc3-c3ccccc3)cc21 ; CC1(C)c2ccccc2-c2ccc(N(c3ccc(-c4ccc5c(c4)sc4ccccc45)cc3)c3ccccc3-c3ccccc3-c3ccccc3-c3ccccc3)cc21]

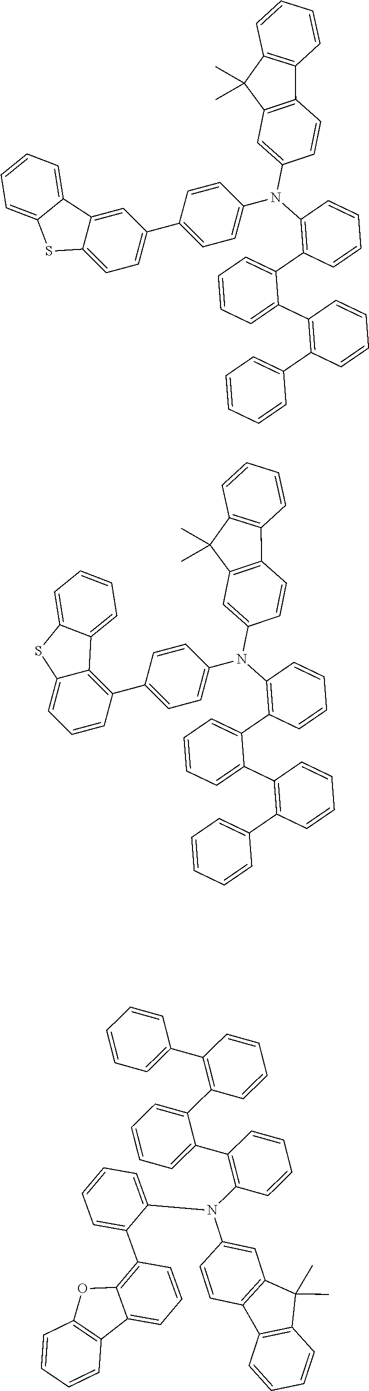

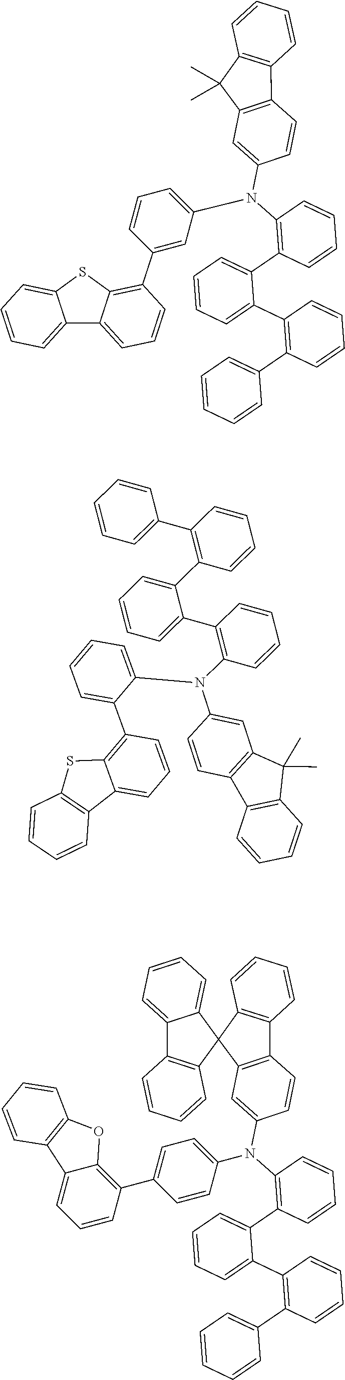 [SMILES: CC1(C)c2ccccc2-c2ccc(N(c3cccc(-c4cccc5c4sc4ccccc45)c3)c3ccccc3-c3ccccc3-c3ccccc3-c3ccccc3)cc21 ; CC1(C)c2ccccc2-c2ccc(N(c3ccccc3-c3ccccc3-c3ccccc3-c3ccccc3)c3ccccc3-c3cccc4c3sc3ccccc34)cc21 ; c1ccc(-c2ccccc2-c2ccccc2-c2ccccc2N(c2ccc(-c3cccc4c3oc3ccccc34)cc2)c2ccc3c(c2)C2(c4ccccc4-c4ccccc42)c2ccccc2-3)cc1]

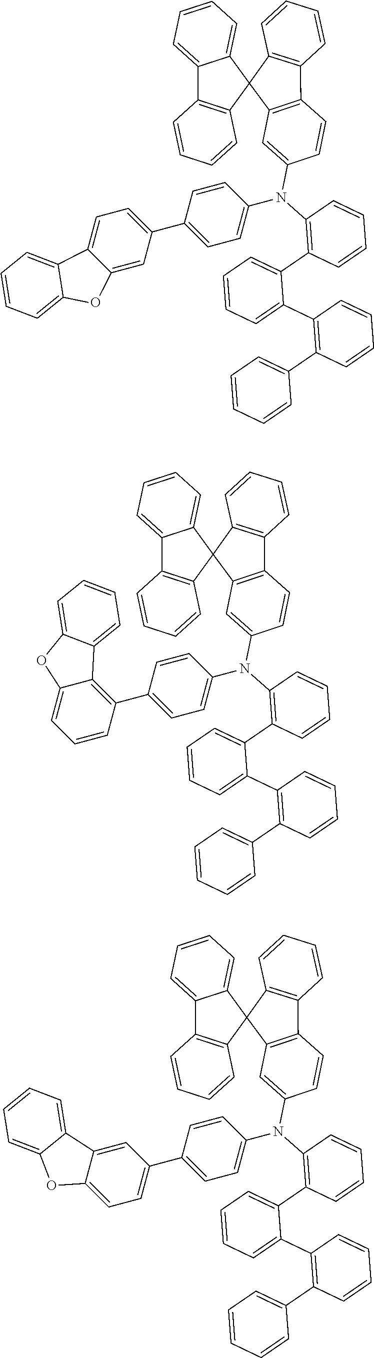

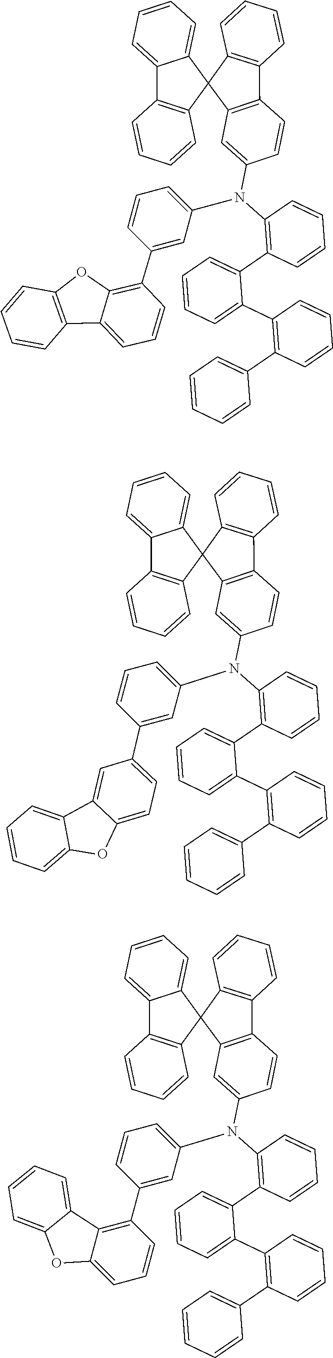

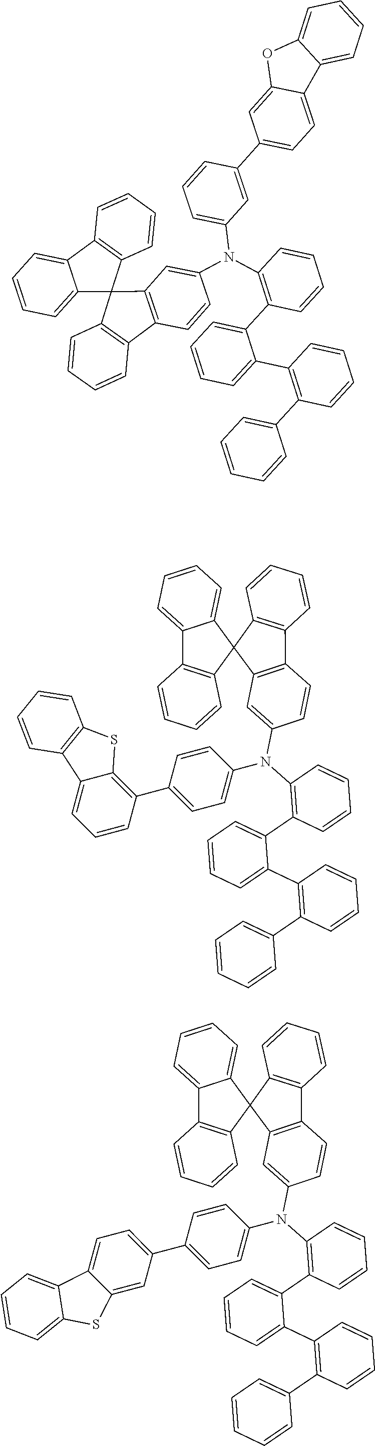 [SMILES: c1ccc(-c2ccccc2-c2ccccc2-c2ccccc2N(c2cccc(-c3ccc4c(c3)oc3ccccc34)c2)c2ccc3c(c2)C2(c4ccccc4-c4ccccc42)c2ccccc2-3)cc1 ; c1ccc(-c2ccccc2-c2ccccc2-c2ccccc2N(c2ccc(-c3cccc4c3sc3ccccc34)cc2)c2ccc3c(c2)C2(c4ccccc4-c4ccccc42)c2ccccc2-3)cc1 ; c1ccc(-c2ccccc2-c2ccccc2-c2ccccc2N(c2ccc(-c3ccc4c(c3)sc3ccccc34)cc2)c2ccc3c(c2)C2(c4ccccc4-c4ccccc42)c2ccccc2-3)cc1]

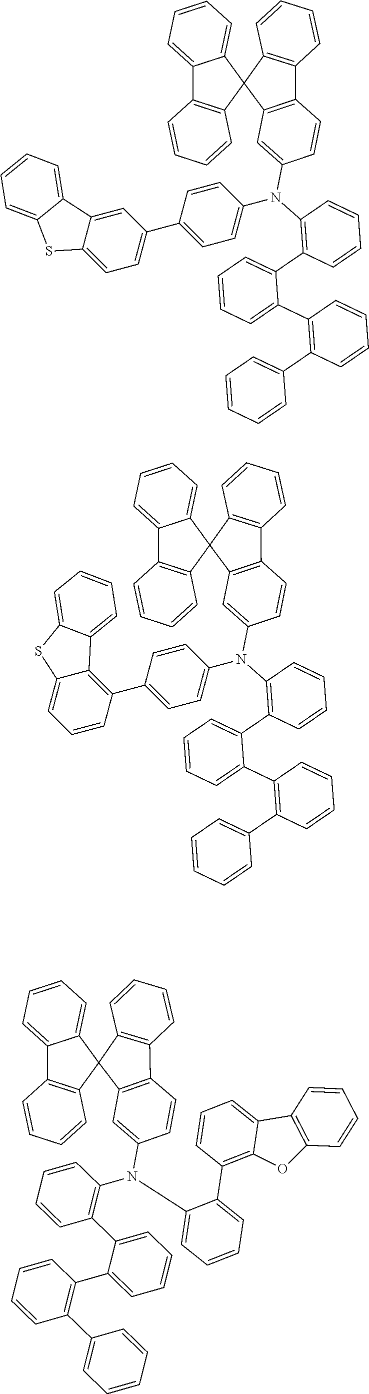

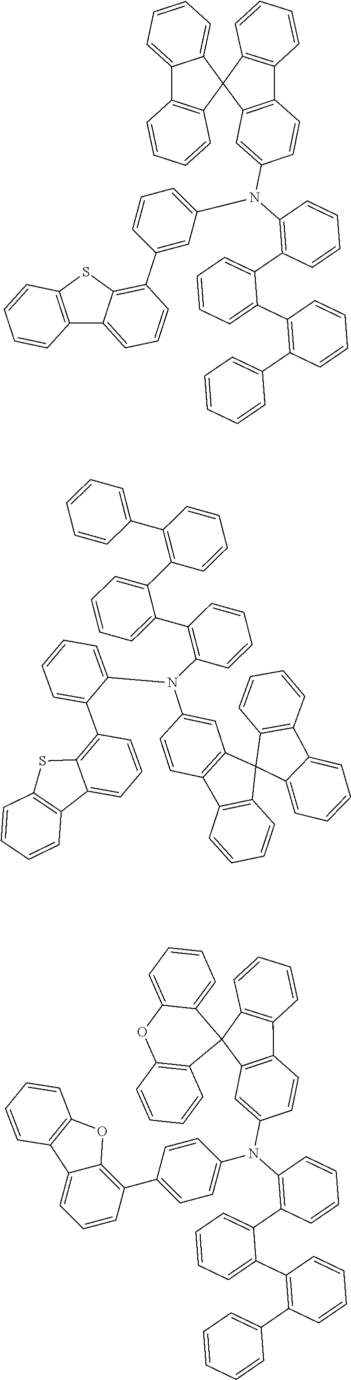 [SMILES: c1ccc(-c2ccccc2-c2ccccc2-c2ccccc2N(c2cccc(-c3cccc4c3sc3ccccc34)c2)c2ccc3c(c2)C2(c4ccccc4-c4ccccc42)c2ccccc2-3)cc1 ; c1ccc(-c2ccccc2-c2ccccc2-c2ccccc2N(c2ccc3c(c2)C2(c4ccccc4-c4ccccc42)c2ccccc2-3)c2ccccc2-c2cccc3c2sc2ccccc23)cc1 ; c1ccc(-c2ccccc2-c2ccccc2-c2ccccc2N(c2ccc(-c3cccc4c3oc3ccccc34)cc2)c2ccc3c(c2)C2(c4ccccc4Oc4ccccc42)c2ccccc2-3)cc1]

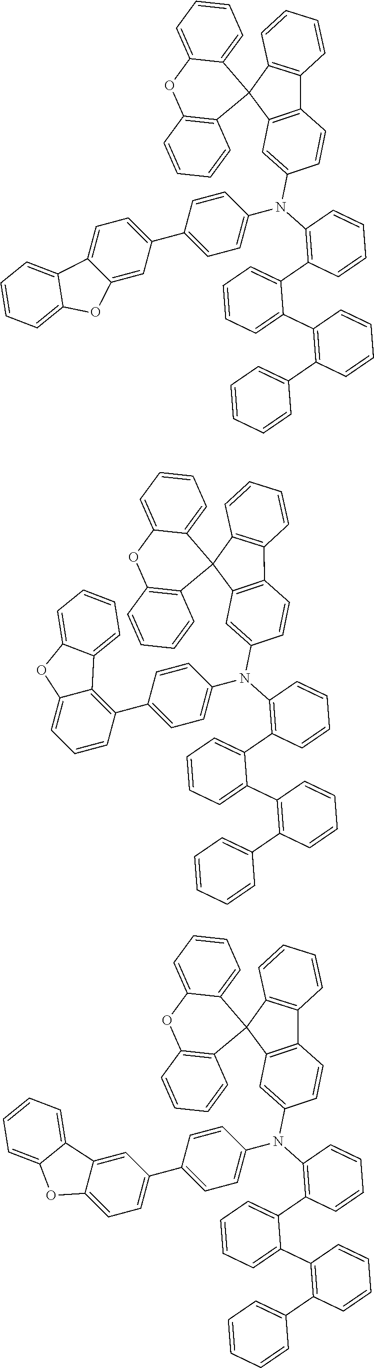

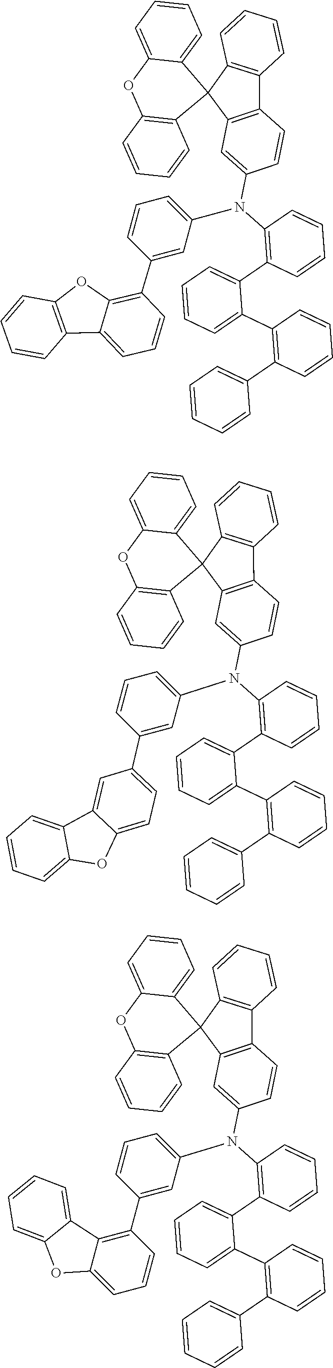

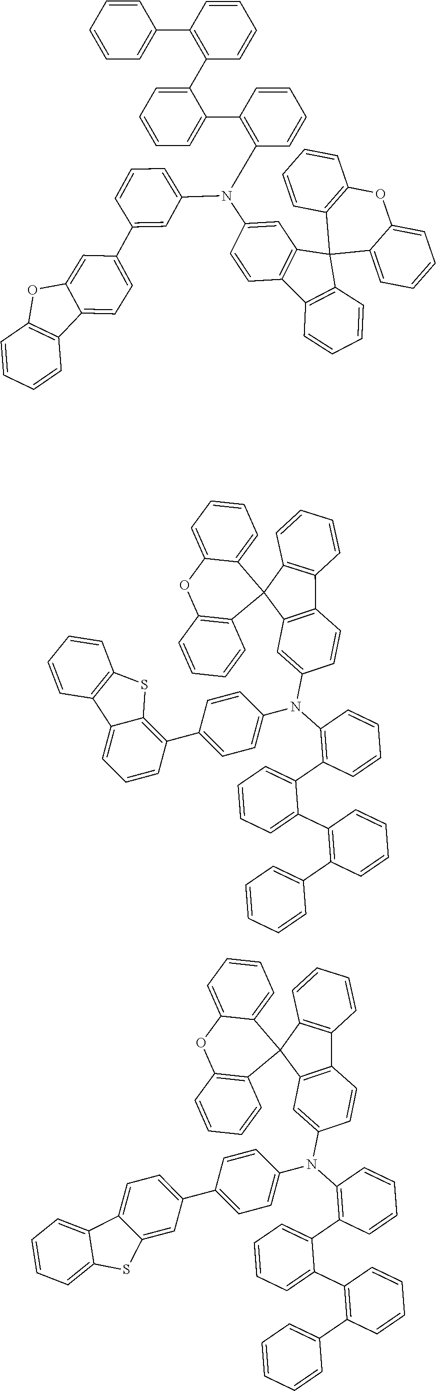

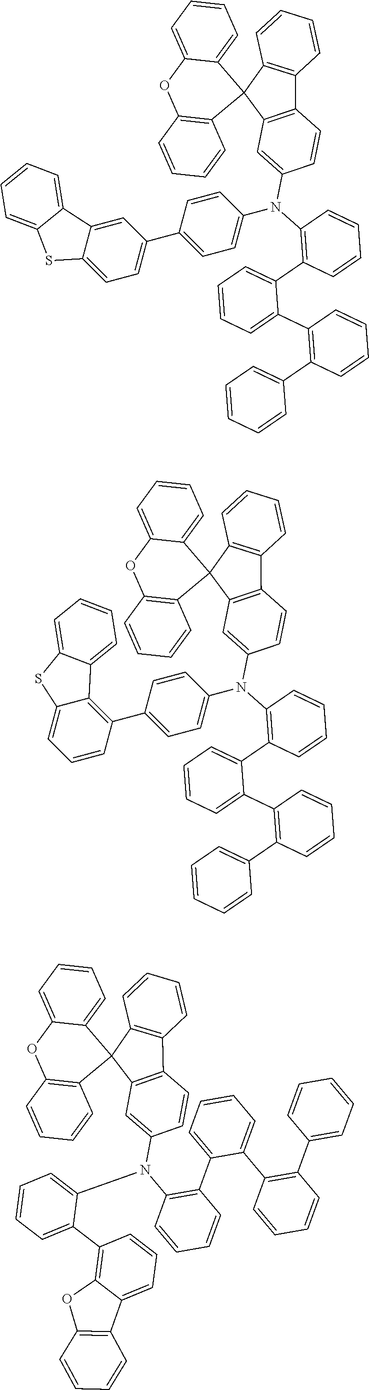 [SMILES: c1ccc(-c2ccccc2-c2ccccc2-c2ccccc2N(c2ccc(-c3ccc4sc5ccccc5c4c3)cc2)c2ccc3c(c2)C2(c4ccccc4Oc4ccccc42)c2ccccc2-3)cc1 ; c1ccc(-c2ccccc2-c2ccccc2-c2ccccc2N(c2ccc(-c3cccc4sc5ccccc5c34)cc2)c2ccc3c(c2)C2(c4ccccc4Oc4ccccc42)c2ccccc2-3)cc1 ; c1ccc(-c2ccccc2-c2ccccc2-c2ccccc2N(c2ccc3c(c2)C2(c4ccccc4Oc4ccccc42)c2ccccc2-3)c2ccccc2-c2cccc3c2oc2ccccc23)cc1]

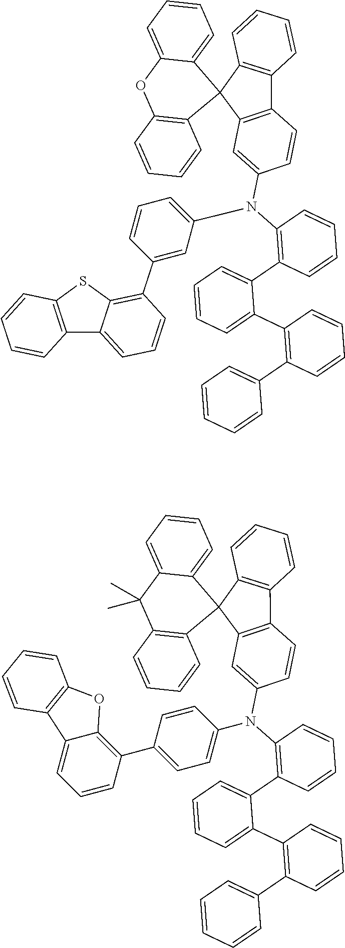 [SMILES: c1ccc(-c2ccccc2-c2ccccc2-c2ccccc2N(c2cccc(-c3cccc4c3sc3ccccc34)c2)c2ccc3c(c2)C2(c4ccccc4Oc4ccccc42)c2ccccc2-3)cc1 ; CC1(C)c2ccccc2C2(c3ccccc3-c3ccc(N(c4ccc(-c5cccc6c5oc5ccccc56)cc4)c4ccccc4-c4ccccc4-c4ccccc4-c4ccccc4)cc32)c2ccccc21]

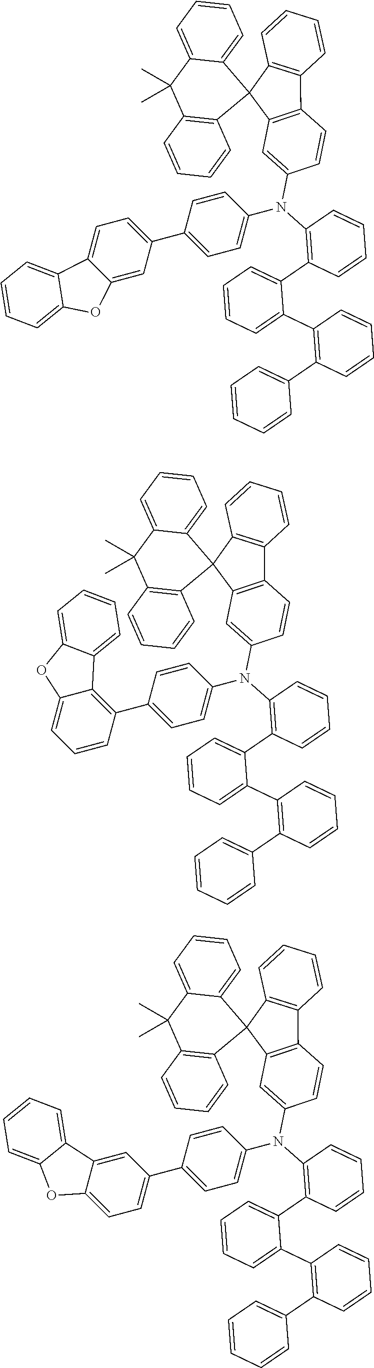

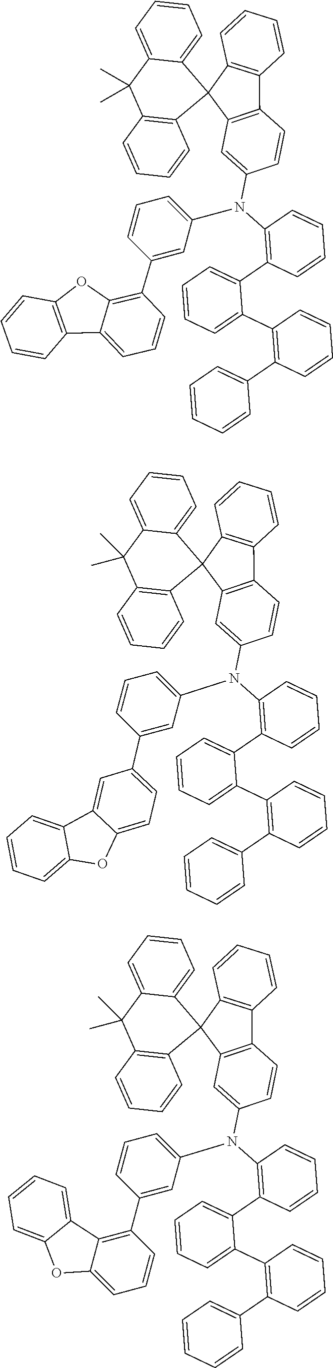

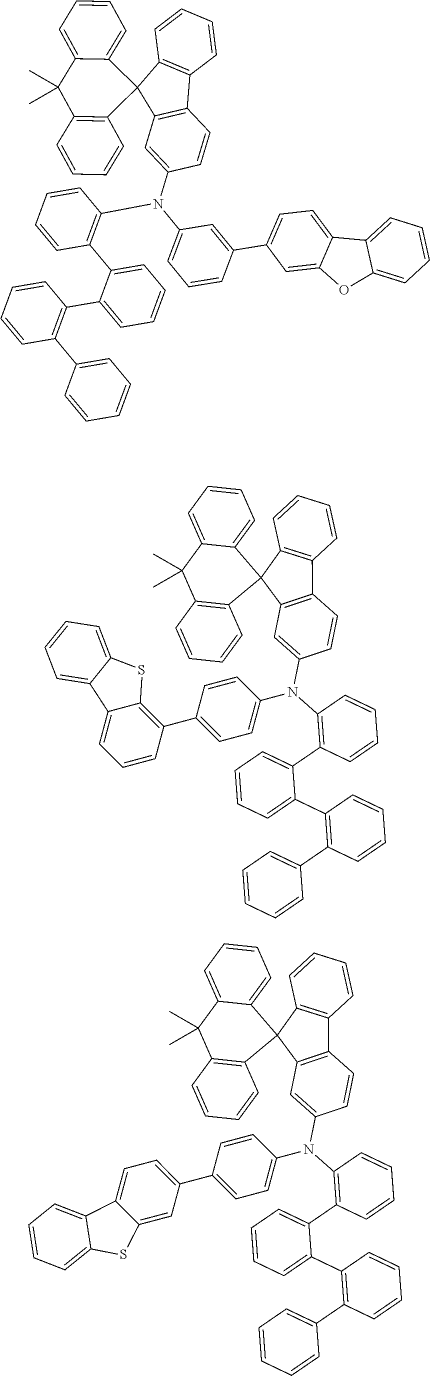

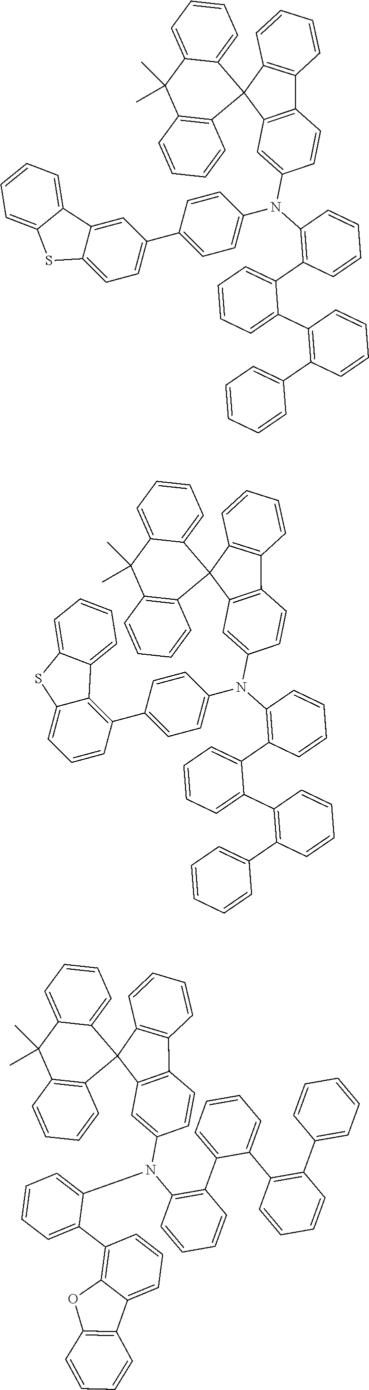 [SMILES: CC1(C)c2ccccc2C2(c3ccccc3-c3ccc(N(c4ccc(-c5ccc6sc7ccccc7c6c5)cc4)c4ccccc4-c4ccccc4-c4ccccc4-c4ccccc4)cc32)c2ccccc21 ; CC1(C)c2ccccc2C2(c3ccccc3-c3ccc(N(c4ccc(-c5cccc6sc7ccccc7c56)cc4)c4ccccc4-c4ccccc4-c4ccccc4-c4ccccc4)cc32)c2ccccc21 ; CC1(C)c2ccccc2C2(c3ccccc3-c3ccc(N(c4ccccc4-c4ccccc4-c4ccccc4-c4ccccc4)c4ccccc4-c4cccc5c4oc4ccccc45)cc32)c2ccccc21]

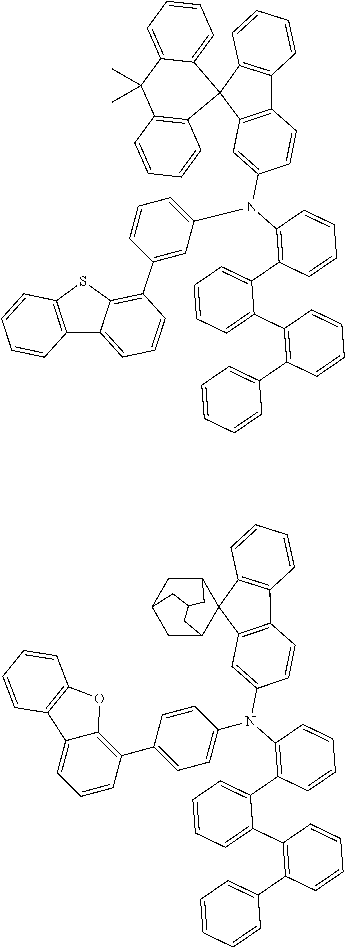 [SMILES: CC1(C)c2ccccc2C2(c3ccccc3-c3ccc(N(c4cccc(-c5cccc6c5sc5ccccc56)c4)c4ccccc4-c4ccccc4-c4ccccc4-c4ccccc4)cc32)c2ccccc21 ; c1ccc(-c2ccccc2-c2ccccc2-c2ccccc2N(c2ccc(-c3cccc4c3oc3ccccc34)cc2)c2ccc3c(c2)C2(c4ccccc4-3)C3CC4CC(C3)CC2C4)cc1]

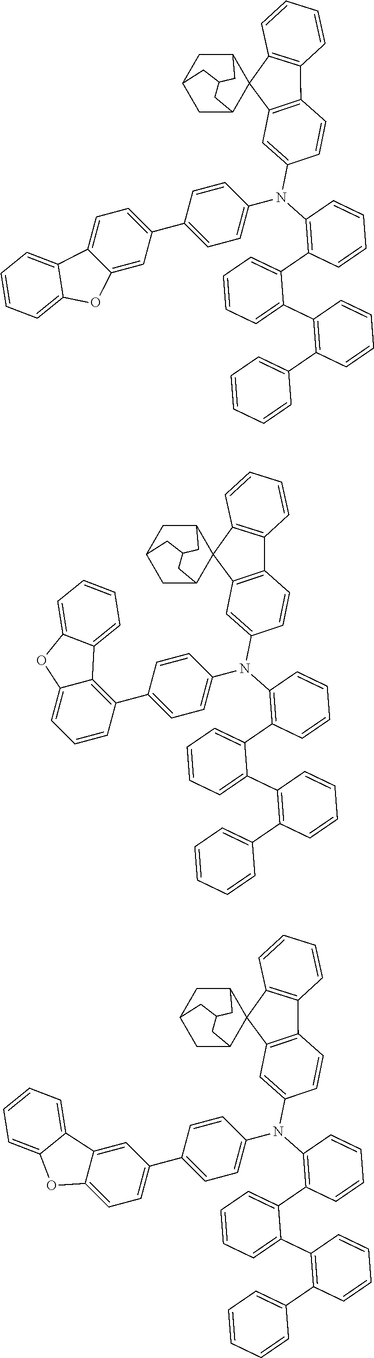 [SMILES: c1ccc(-c2ccccc2-c2ccccc2-c2ccccc2N(c2ccc(-c3ccc4c(c3)oc3ccccc34)cc2)c2ccc3c(c2)C2(c4ccccc4-3)C3CC4CC(C3)CC2C4)cc1 ; c1ccc(-c2ccccc2-c2ccccc2-c2ccccc2N(c2ccc(-c3cccc4oc5ccccc5c34)cc2)c2ccc3c(c2)C2(c4ccccc4-3)C3CC4CC(C3)CC2C4)cc1 ; c1ccc(-c2ccccc2-c2ccccc2-c2ccccc2N(c2ccc(-c3ccc4oc5ccccc5c4c3)cc2)c2ccc3c(c2)C2(c4ccccc4-3)C3CC4CC(C3)CC2C4)cc1]

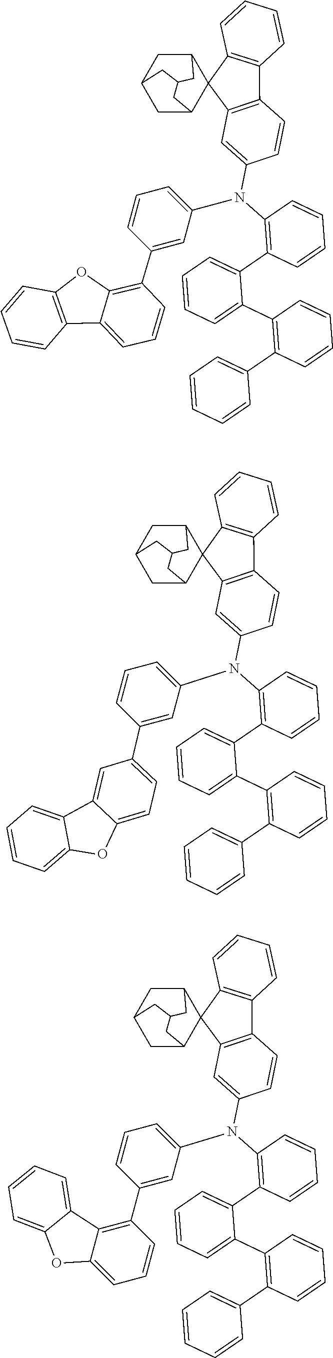

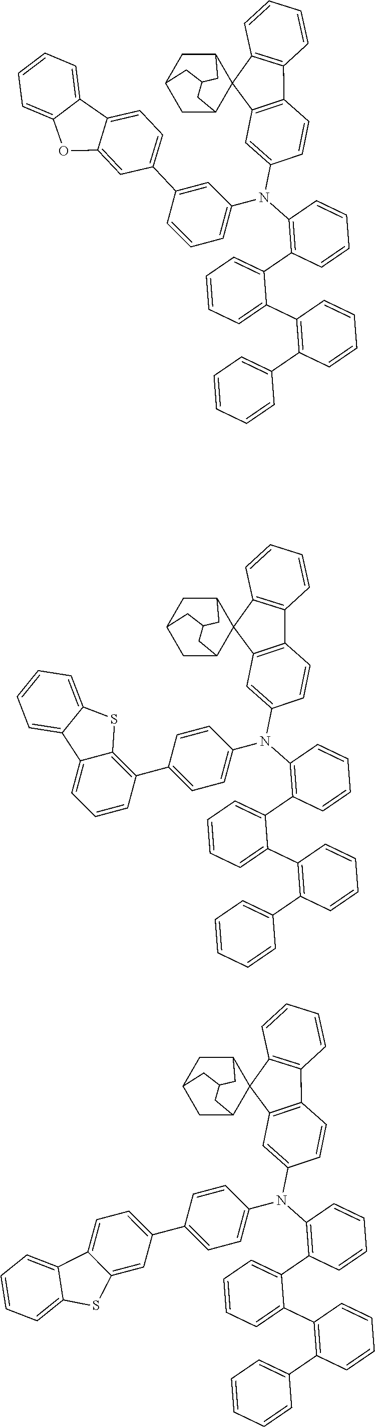 [SMILES: c1ccc(-c2ccccc2-c2ccccc2-c2ccccc2N(c2cccc(-c3ccc4c(c3)oc3ccccc34)c2)c2ccc3c(c2)C2(c4ccccc4-3)C3CC4CC(C3)CC2C4)cc1 ; c1ccc(-c2ccccc2-c2ccccc2-c2ccccc2N(c2ccc(-c3cccc4c3sc3ccccc34)cc2)c2ccc3c(c2)C2(c4ccccc4-3)C3CC4CC(C3)CC2C4)cc1 ; c1ccc(-c2ccccc2-c2ccccc2-c2ccccc2N(c2ccc(-c3ccc4c(c3)sc3ccccc34)cc2)c2ccc3c(c2)C2(c4ccccc4-3)C3CC4CC(C3)CC2C4)cc1]

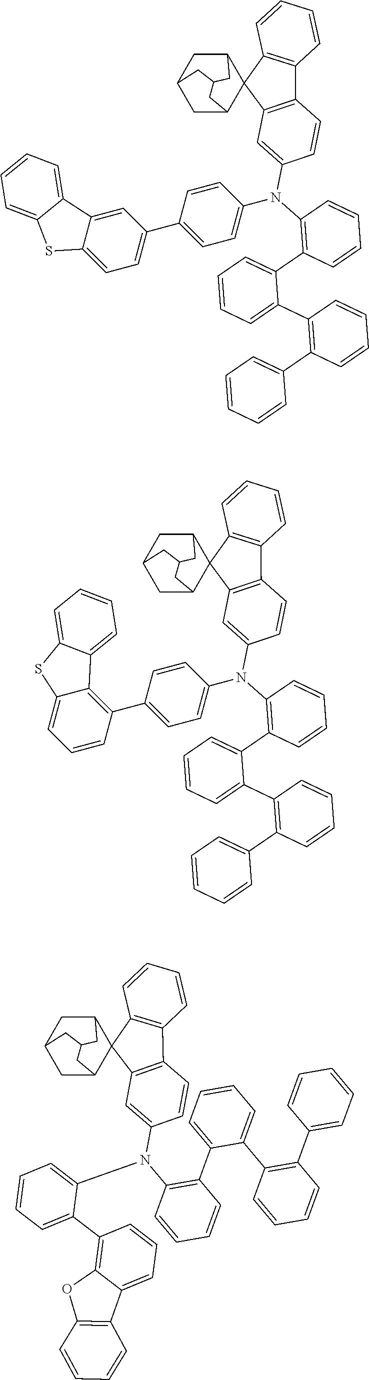 [SMILES: c1ccc(-c2ccccc2-c2ccccc2-c2ccccc2N(c2ccc(-c3ccc4sc5ccccc5c4c3)cc2)c2ccc3c(c2)C2(c4ccccc4-3)C3CC4CC(C3)CC2C4)cc1 ; c1ccc(-c2ccccc2-c2ccccc2-c2ccccc2N(c2ccc(-c3cccc4sc5ccccc5c34)cc2)c2ccc3c(c2)C2(c4ccccc4-3)C3CC4CC(C3)CC2C4)cc1 ; c1ccc(-c2ccccc2-c2ccccc2-c2ccccc2N(c2ccc3c(c2)C2(c4ccccc4-3)C3CC4CC(C3)CC2C4)c2ccccc2-c2cccc3c2oc2ccccc23)cc1]

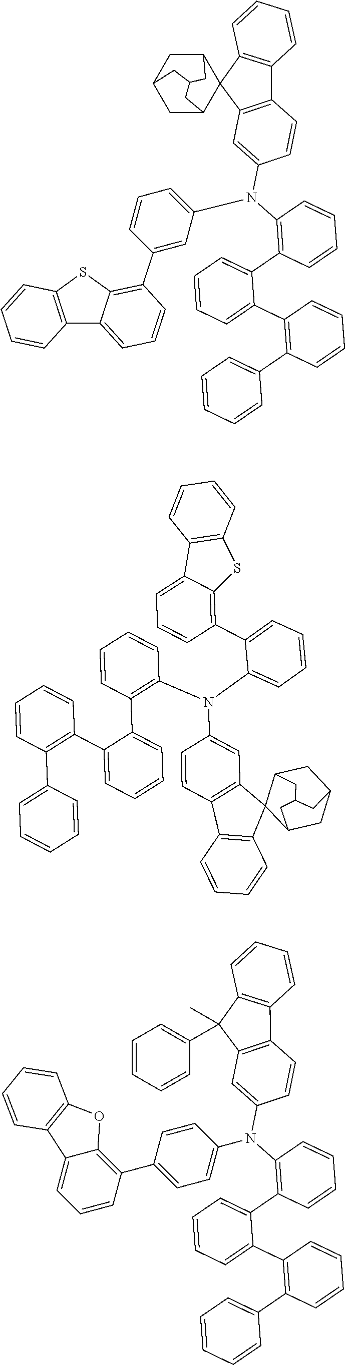 [SMILES: c1ccc(-c2ccccc2-c2ccccc2-c2ccccc2N(c2cccc(-c3cccc4c3sc3ccccc34)c2)c2ccc3c(c2)C2(c4ccccc4-3)C3CC4CC(C3)CC2C4)cc1 ; c1ccc(-c2ccccc2-c2ccccc2-c2ccccc2N(c2ccc3c(c2)C2(c4ccccc4-3)C3CC4CC(C3)CC2C4)c2ccccc2-c2cccc3c2sc2ccccc23)cc1 ; CC1(c2ccccc2)c2ccccc2-c2ccc(N(c3ccc(-c4cccc5c4oc4ccccc45)cc3)c3ccccc3-c3ccccc3-c3ccccc3-c3ccccc3)cc21]

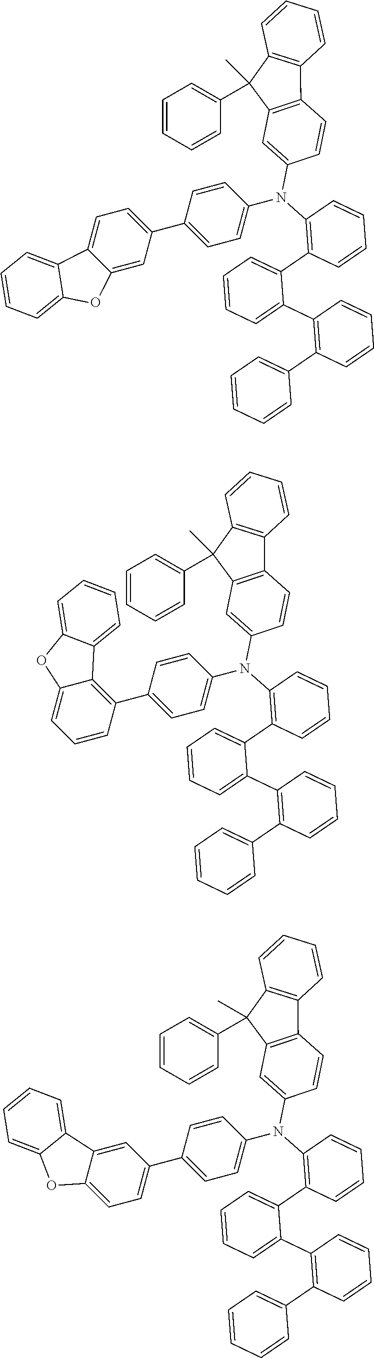 [SMILES: CC1(c2ccccc2)c2ccccc2-c2ccc(N(c3ccc(-c4ccc5c(c4)oc4ccccc45)cc3)c3ccccc3-c3ccccc3-c3ccccc3-c3ccccc3)cc21 ; CC1(c2ccccc2)c2ccccc2-c2ccc(N(c3ccc(-c4cccc5oc6ccccc6c45)cc3)c3ccccc3-c3ccccc3-c3ccccc3-c3ccccc3)cc21 ; CC1(c2ccccc2)c2ccccc2-c2ccc(N(c3ccc(-c4ccc5oc6ccccc6c5c4)cc3)c3ccccc3-c3ccccc3-c3ccccc3-c3ccccc3)cc21]

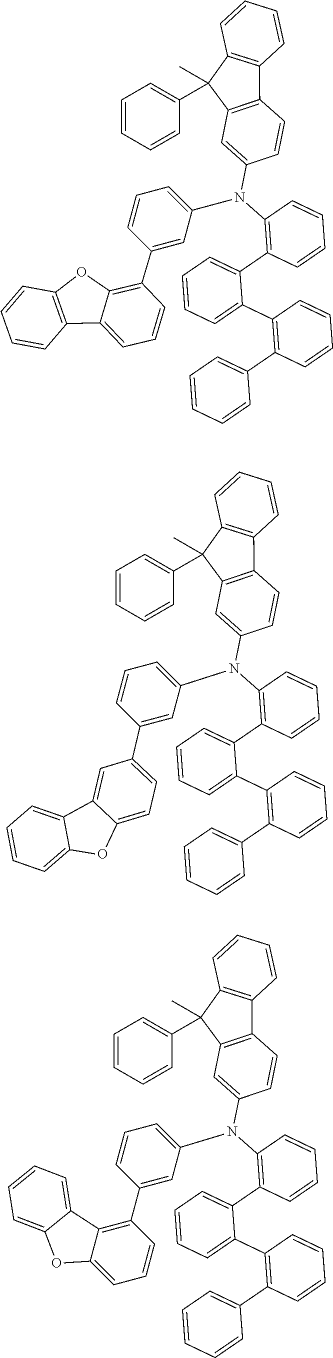

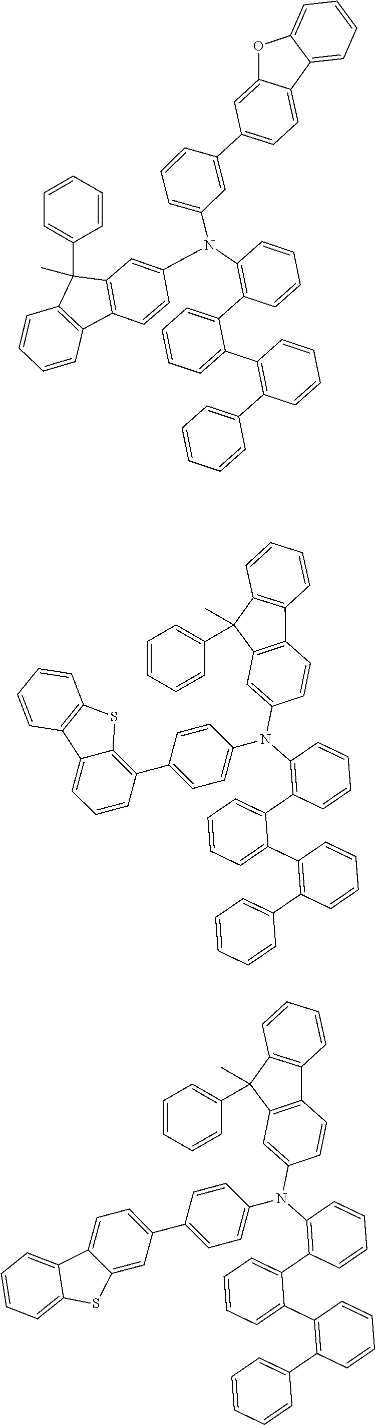 [SMILES: CC1(c2ccccc2)c2ccccc2-c2ccc(N(c3cccc(-c4ccc5c(c4)oc4ccccc45)c3)c3ccccc3-c3ccccc3-c3ccccc3-c3ccccc3)cc21 ; CC1(c2ccccc2)c2ccccc2-c2ccc(N(c3ccc(-c4cccc5c4sc4ccccc45)cc3)c3ccccc3-c3ccccc3-c3ccccc3-c3ccccc3)cc21 ; CC1(c2ccccc2)c2ccccc2-c2ccc(N(c3ccc(-c4ccc5c(c4)sc4ccccc45)cc3)c3ccccc3-c3ccccc3-c3ccccc3-c3ccccc3)cc21]

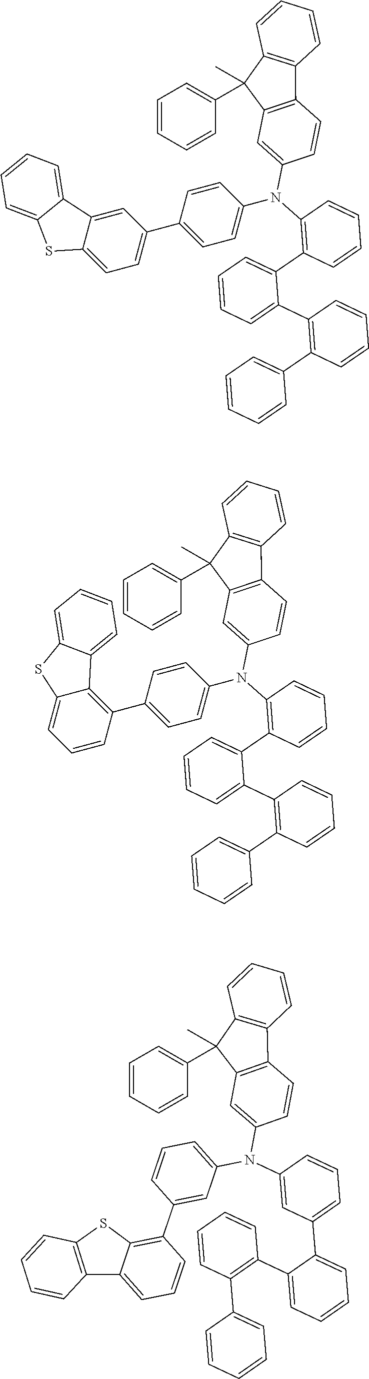 [SMILES: CC1(c2ccccc2)c2ccccc2-c2ccc(N(c3ccc(-c4ccc5sc6ccccc6c5c4)cc3)c3ccccc3-c3ccccc3-c3ccccc3-c3ccccc3)cc21 ; CC1(c2ccccc2)c2ccccc2-c2ccc(N(c3ccc(-c4cccc5sc6ccccc6c45)cc3)c3ccccc3-c3ccccc3-c3ccccc3-c3ccccc3)cc21 ; CC1(c2ccccc2)c2ccccc2-c2ccc(N(c3cccc(-c4ccccc4-c4ccccc4-c4ccccc4)c3)c3cccc(-c4cccc5c4sc4ccccc45)c3)cc21]

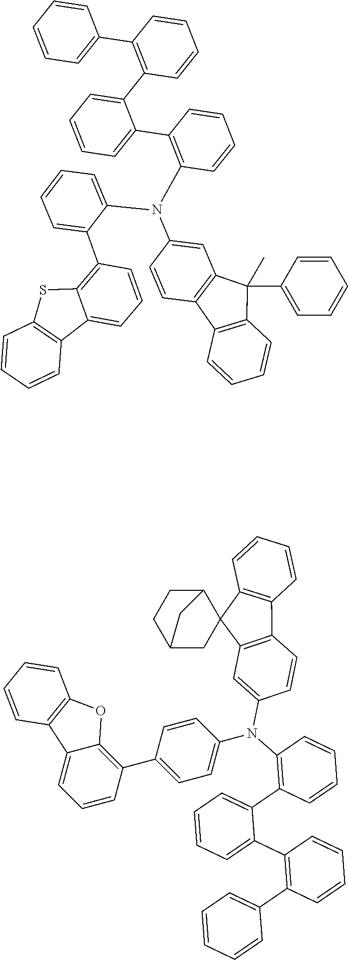 [SMILES: CC1(c2ccccc2)c2ccccc2-c2ccc(N(c3ccccc3-c3ccccc3-c3ccccc3-c3ccccc3)c3ccccc3-c3cccc4c3sc3ccccc34)cc21 ; c1ccc(-c2ccccc2-c2ccccc2-c2ccccc2N(c2ccc(-c3cccc4c3oc3ccccc34)cc2)c2ccc3c(c2)C2(CC4CCC2C4)c2ccccc2-3)cc1]

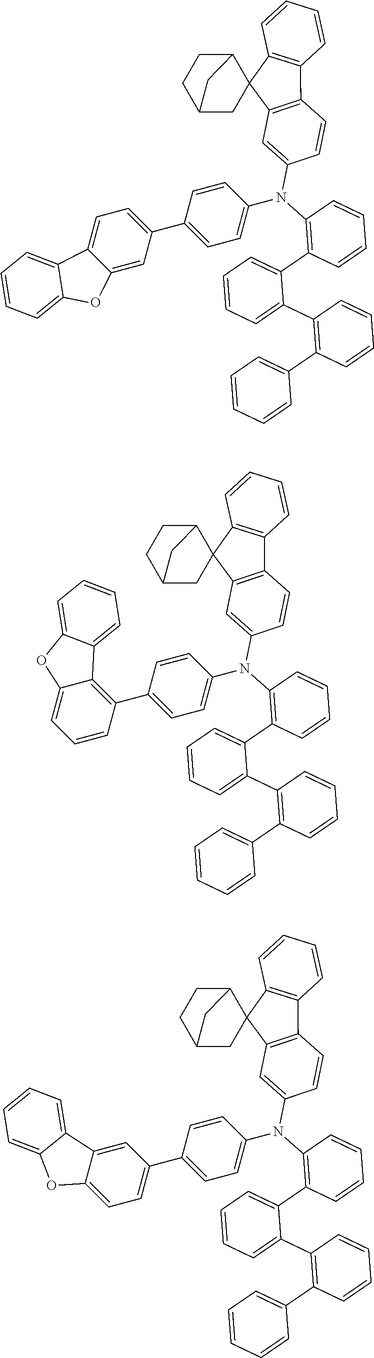 [SMILES: c1ccc(-c2ccccc2-c2ccccc2-c2ccccc2N(c2ccc(-c3ccc4c(c3)oc3ccccc34)cc2)c2ccc3c(c2)C2(CC4CCC2C4)c2ccccc2-3)cc1 ; c1ccc(-c2ccccc2-c2ccccc2-c2ccccc2N(c2ccc(-c3cccc4oc5ccccc5c34)cc2)c2ccc3c(c2)C2(CC4CCC2C4)c2ccccc2-3)cc1 ; c1ccc(-c2ccccc2-c2ccccc2-c2ccccc2N(c2ccc(-c3ccc4oc5ccccc5c4c3)cc2)c2ccc3c(c2)C2(CC4CCC2C4)c2ccccc2-3)cc1]

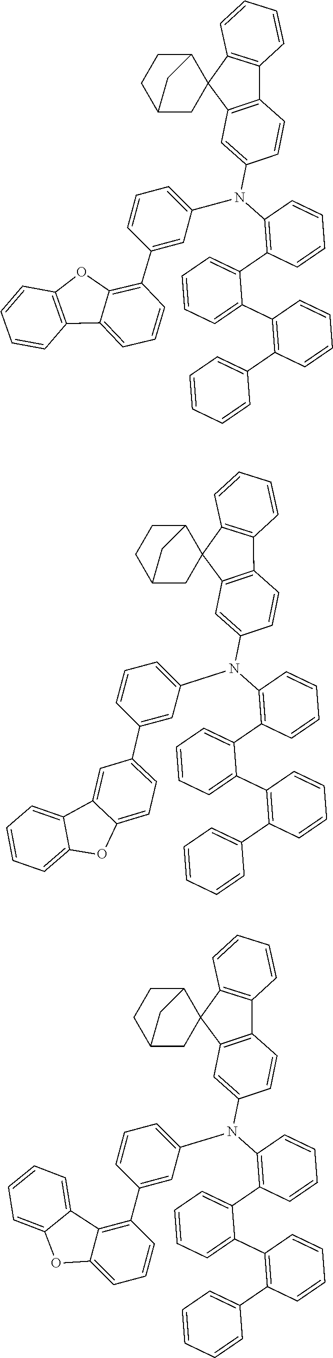

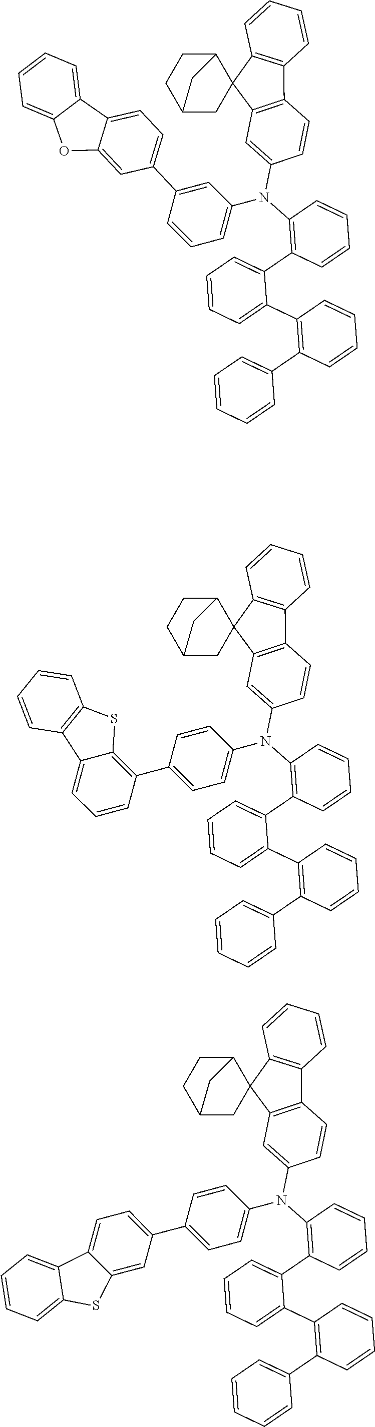 [SMILES: c1ccc(-c2ccccc2-c2ccccc2-c2ccccc2N(c2cccc(-c3ccc4c(c3)oc3ccccc34)c2)c2ccc3c(c2)C2(CC4CCC2C4)c2ccccc2-3)cc1 ; c1ccc(-c2ccccc2-c2ccccc2-c2ccccc2N(c2ccc(-c3cccc4c3sc3ccccc34)cc2)c2ccc3c(c2)C2(CC4CCC2C4)c2ccccc2-3)cc1 ; c1ccc(-c2ccccc2-c2ccccc2-c2ccccc2N(c2ccc(-c3ccc4c(c3)sc3ccccc34)cc2)c2ccc3c(c2)C2(CC4CCC2C4)c2ccccc2-3)cc1]

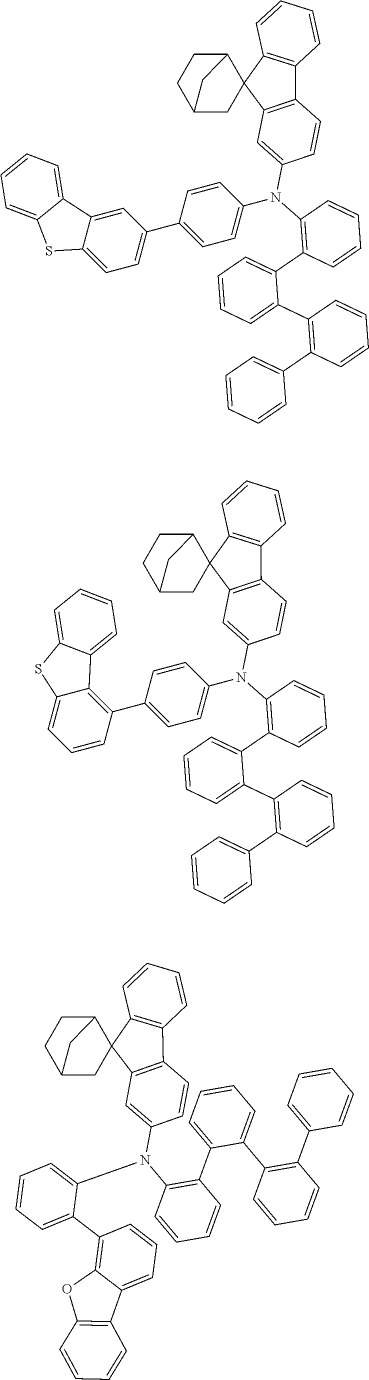 [SMILES: c1ccc(-c2ccccc2-c2ccccc2-c2ccccc2N(c2ccc(-c3ccc4sc5ccccc5c4c3)cc2)c2ccc3c(c2)C2(CC4CCC2C4)c2ccccc2-3)cc1 ; c1ccc(-c2ccccc2-c2ccccc2-c2ccccc2N(c2ccc(-c3cccc4sc5ccccc5c34)cc2)c2ccc3c(c2)C2(CC4CCC2C4)c2ccccc2-3)cc1 ; c1ccc(-c2ccccc2-c2ccccc2-c2ccccc2N(c2ccc3c(c2)C2(CC4CCC2C4)c2ccccc2-3)c2ccccc2-c2cccc3c2oc2ccccc23)cc1]

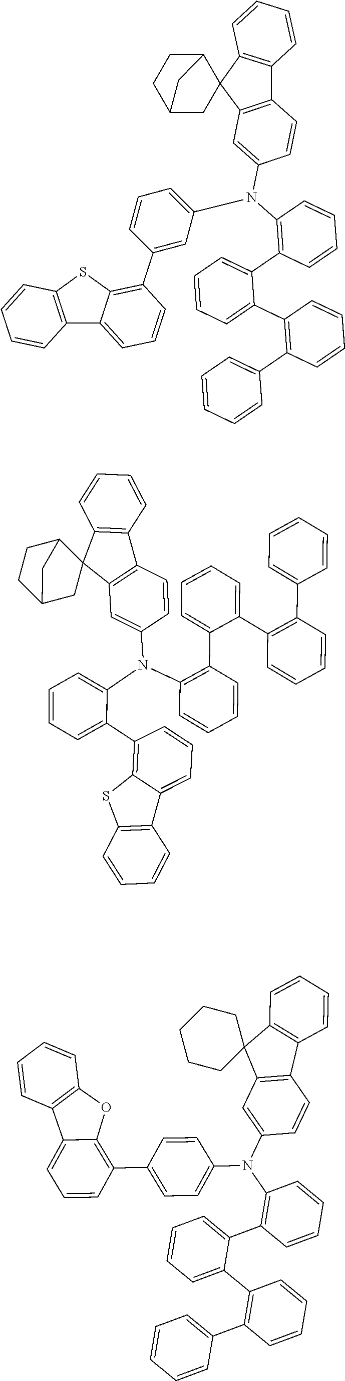 [SMILES: c1ccc(-c2ccccc2-c2ccccc2-c2ccccc2N(c2cccc(-c3cccc4c3sc3ccccc34)c2)c2ccc3c(c2)C2(CC4CCC2C4)c2ccccc2-3)cc1 ; c1ccc(-c2ccccc2-c2ccccc2-c2ccccc2N(c2ccc3c(c2)C2(CC4CCC2C4)c2ccccc2-3)c2ccccc2-c2cccc3c2sc2ccccc23)cc1 ; c1ccc(-c2ccccc2-c2ccccc2-c2ccccc2N(c2ccc(-c3cccc4c3oc3ccccc34)cc2)c2ccc3c(c2)C2(CCCCC2)c2ccccc2-3)cc1]

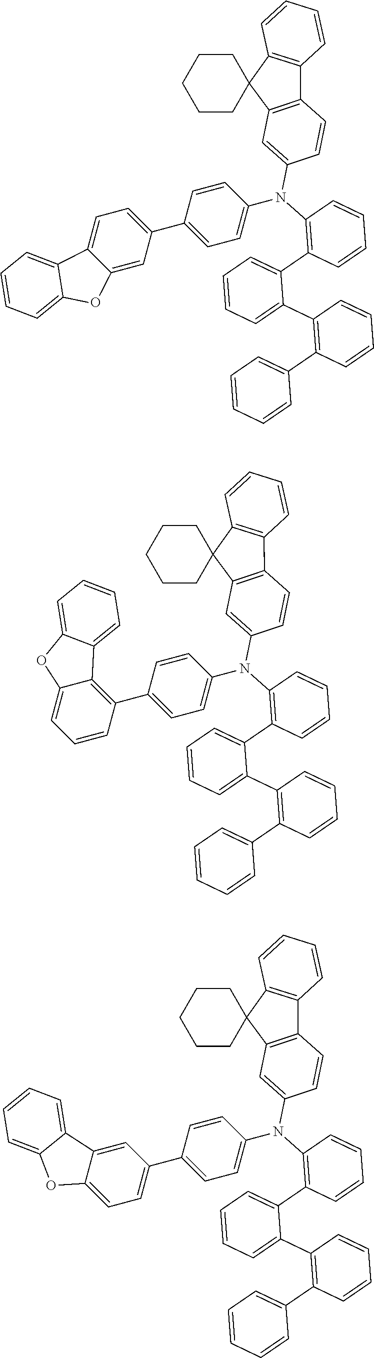 [SMILES: c1ccc(-c2ccccc2-c2ccccc2-c2ccccc2N(c2ccc(-c3ccc4c(c3)oc3ccccc34)cc2)c2ccc3c(c2)C2(CCCCC2)c2ccccc2-3)cc1 ; c1ccc(-c2ccccc2-c2ccccc2-c2ccccc2N(c2ccc(-c3cccc4oc5ccccc5c34)cc2)c2ccc3c(c2)C2(CCCCC2)c2ccccc2-3)cc1 ; c1ccc(-c2ccccc2-c2ccccc2-c2ccccc2N(c2ccc(-c3ccc4oc5ccccc5c4c3)cc2)c2ccc3c(c2)C2(CCCCC2)c2ccccc2-3)cc1]

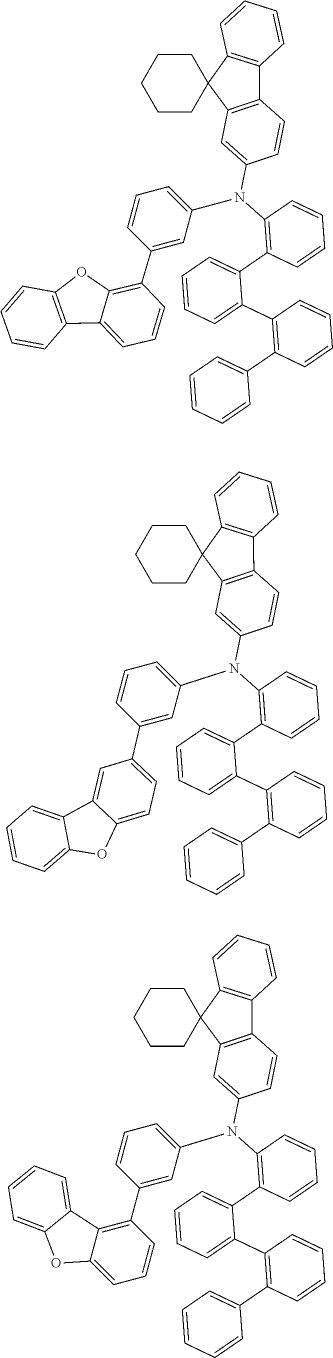

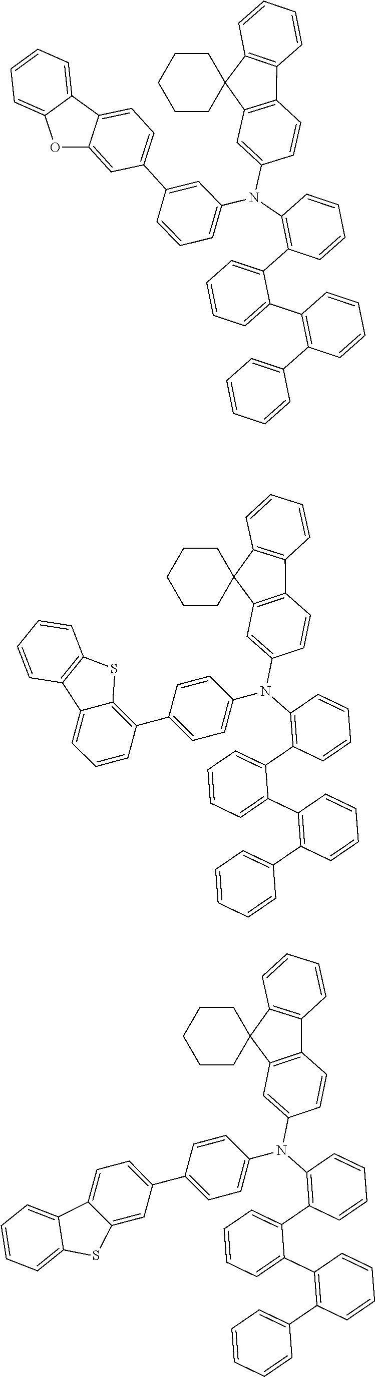 [SMILES: c1ccc(-c2ccccc2-c2ccccc2-c2ccccc2N(c2cccc(-c3ccc4c(c3)oc3ccccc34)c2)c2ccc3c(c2)C2(CCCCC2)c2ccccc2-3)cc1 ; c1ccc(-c2ccccc2-c2ccccc2-c2ccccc2N(c2ccc(-c3cccc4c3sc3ccccc34)cc2)c2ccc3c(c2)C2(CCCCC2)c2ccccc2-3)cc1 ; c1ccc(-c2ccccc2-c2ccccc2-c2ccccc2N(c2ccc(-c3ccc4c(c3)sc3ccccc34)cc2)c2ccc3c(c2)C2(CCCCC2)c2ccccc2-3)cc1]

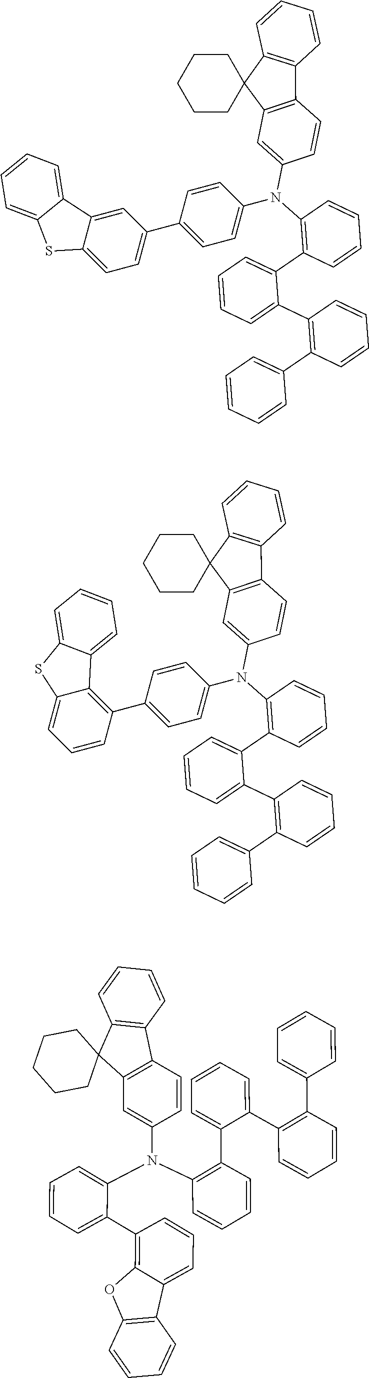 [SMILES: c1ccc(-c2ccccc2-c2ccccc2-c2ccccc2N(c2ccc(-c3ccc4sc5ccccc5c4c3)cc2)c2ccc3c(c2)C2(CCCCC2)c2ccccc2-3)cc1 ; c1ccc(-c2ccccc2-c2ccccc2-c2ccccc2N(c2ccc(-c3cccc4sc5ccccc5c34)cc2)c2ccc3c(c2)C2(CCCCC2)c2ccccc2-3)cc1 ; c1ccc(-c2ccccc2-c2ccccc2-c2ccccc2N(c2ccc3c(c2)C2(CCCCC2)c2ccccc2-3)c2ccccc2-c2cccc3c2oc2ccccc23)cc1]

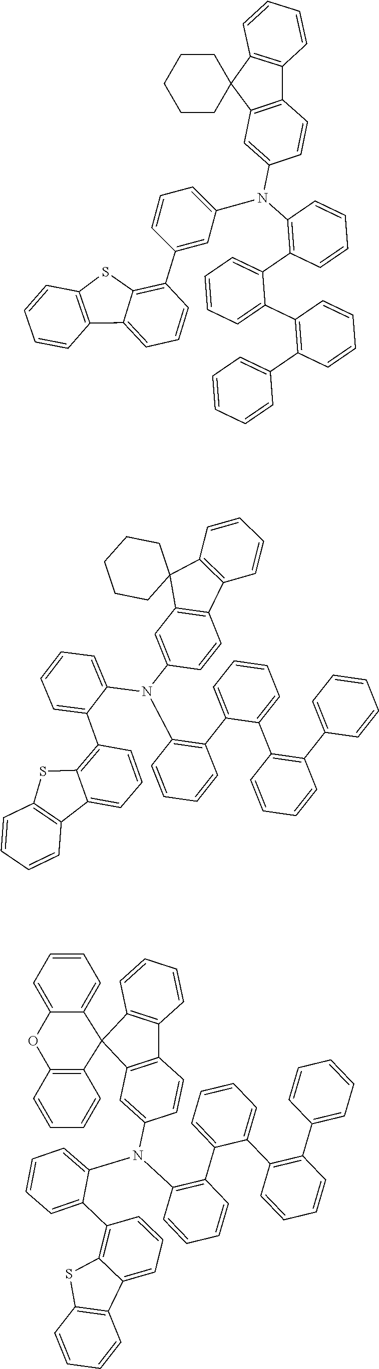 [SMILES: c1ccc(-c2ccccc2-c2ccccc2-c2ccccc2N(c2cccc(-c3cccc4c3sc3ccccc34)c2)c2ccc3c(c2)C2(CCCCC2)c2ccccc2-3)cc1 ; c1ccc(-c2ccccc2-c2ccccc2-c2ccccc2N(c2ccc3c(c2)C2(CCCCC2)c2ccccc2-3)c2ccccc2-c2cccc3c2sc2ccccc23)cc1 ; c1ccc(-c2ccccc2-c2ccccc2-c2ccccc2N(c2ccc3c(c2)C2(c4ccccc4Oc4ccccc42)c2ccccc2-3)c2ccccc2-c2cccc3c2sc2ccccc23)cc1]

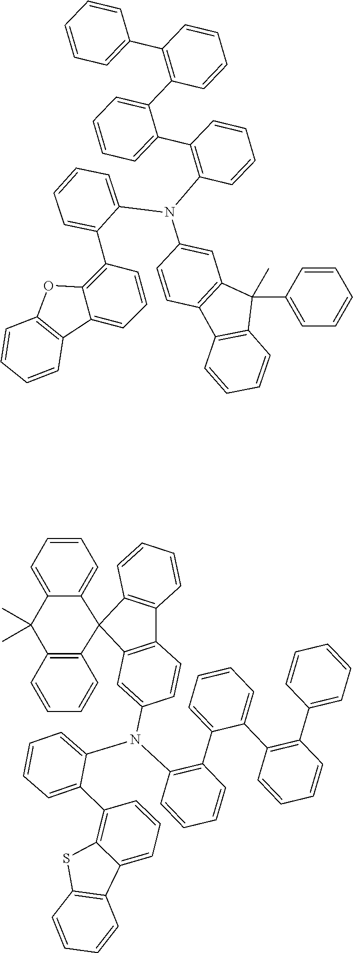 [SMILES: CC1(c2ccccc2)c2ccccc2-c2ccc(N(c3ccccc3-c3ccccc3-c3ccccc3-c3ccccc3)c3ccccc3-c3cccc4c3oc3ccccc34)cc21 ; CC1(C)c2ccccc2C2(c3ccccc3-c3ccc(N(c4ccccc4-c4ccccc4-c4ccccc4-c4ccccc4)c4ccccc4-c4cccc5c4sc4ccccc45)cc32)c2ccccc21]

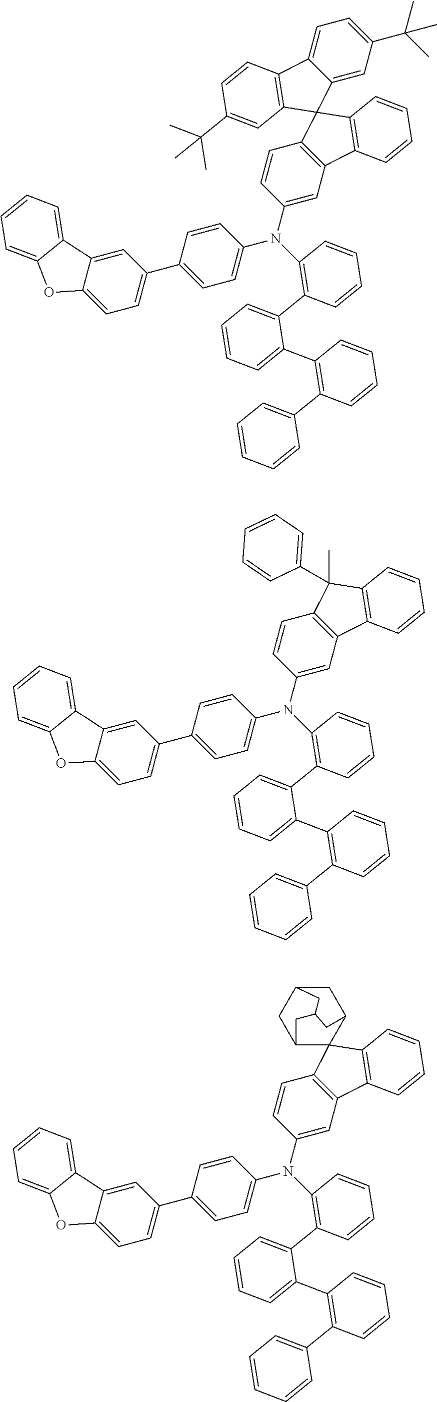

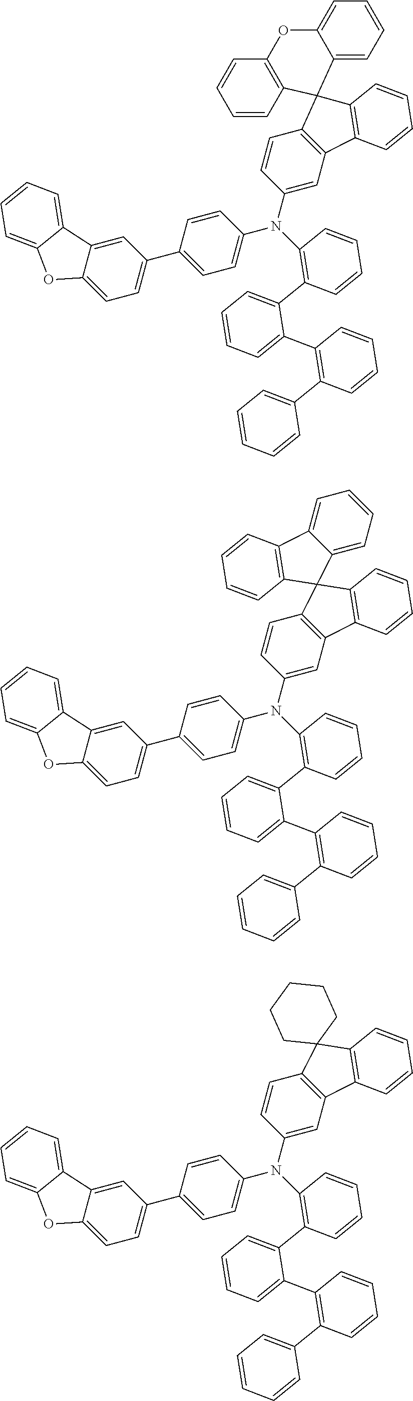

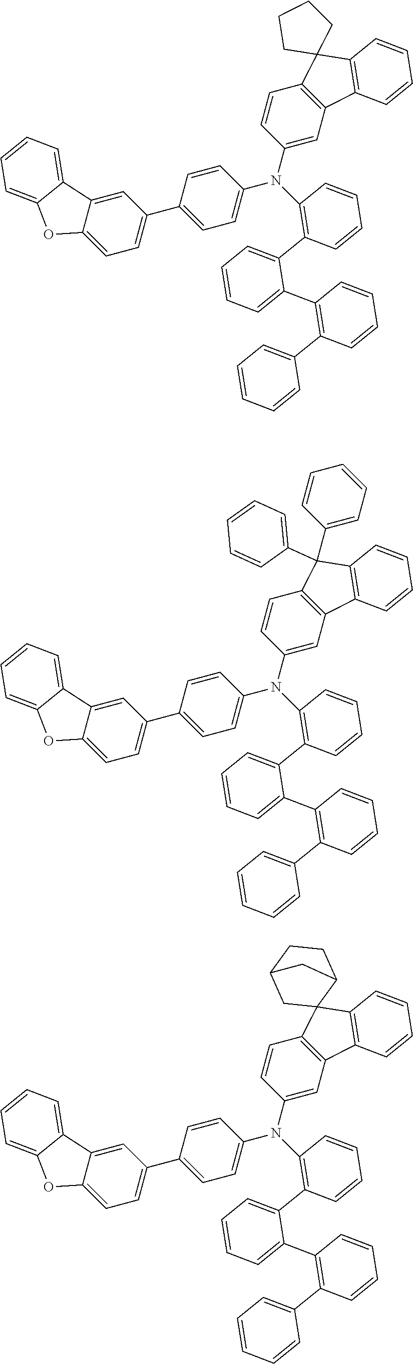

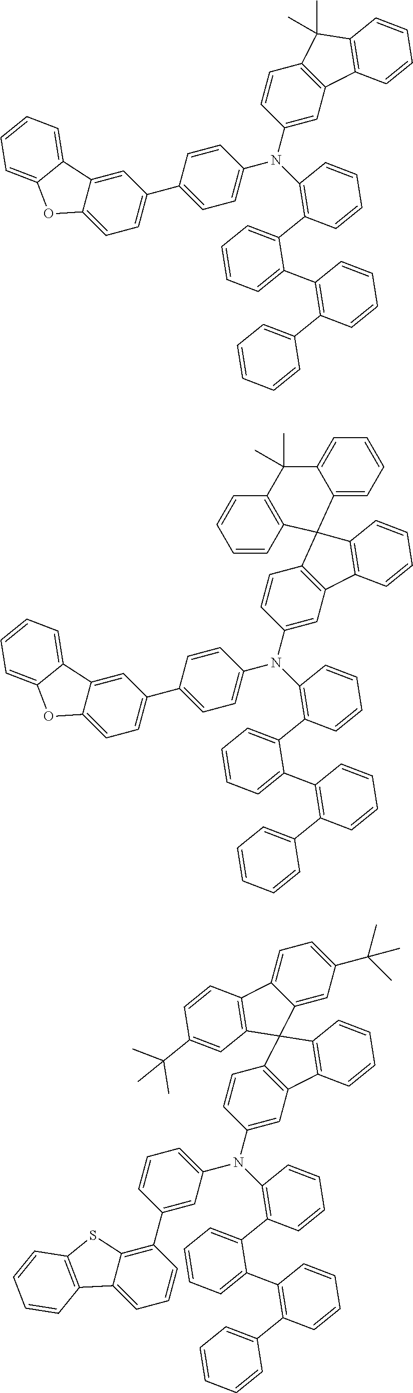

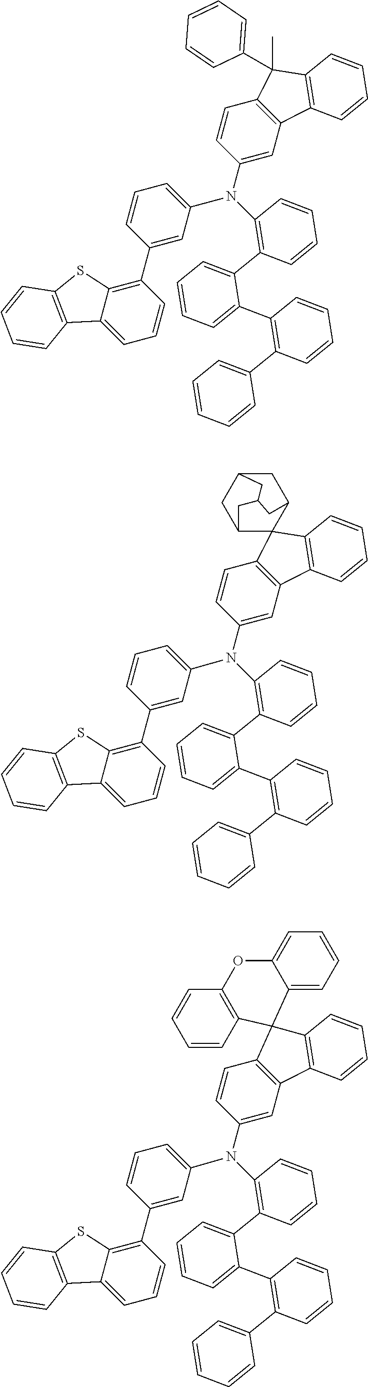 [SMILES: CC1(c2ccccc2)c2ccccc2-c2cc(N(c3cccc(-c4cccc5c4sc4ccccc45)c3)c3ccccc3-c3ccccc3-c3ccccc3-c3ccccc3)ccc21 ; c1ccc(-c2ccccc2-c2ccccc2-c2ccccc2N(c2cccc(-c3cccc4c3sc3ccccc34)c2)c2ccc3c(c2)-c2ccccc2C32C3CC4CC(C3)CC2C4)cc1 ; c1ccc(-c2ccccc2-c2ccccc2-c2ccccc2N(c2cccc(-c3cccc4c3sc3ccccc34)c2)c2ccc3c(c2)-c2ccccc2C32c3ccccc3Oc3ccccc32)cc1]

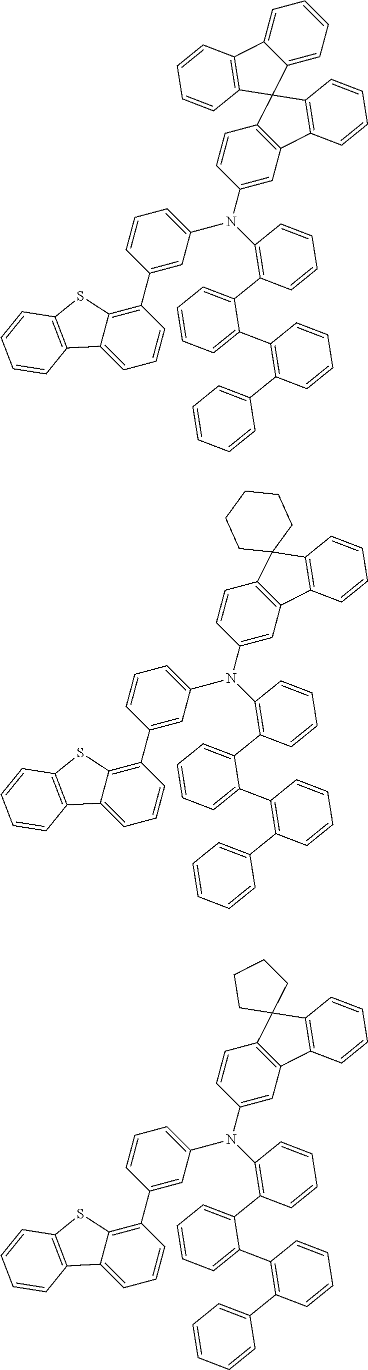

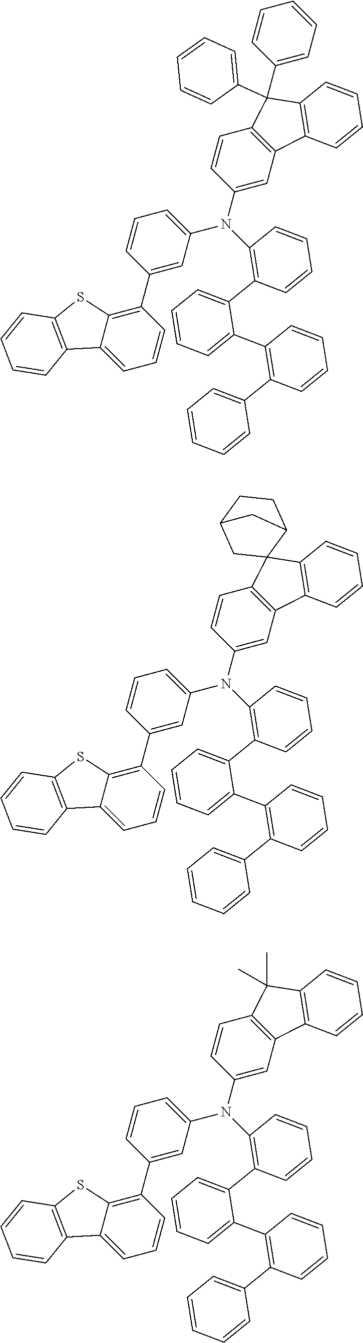

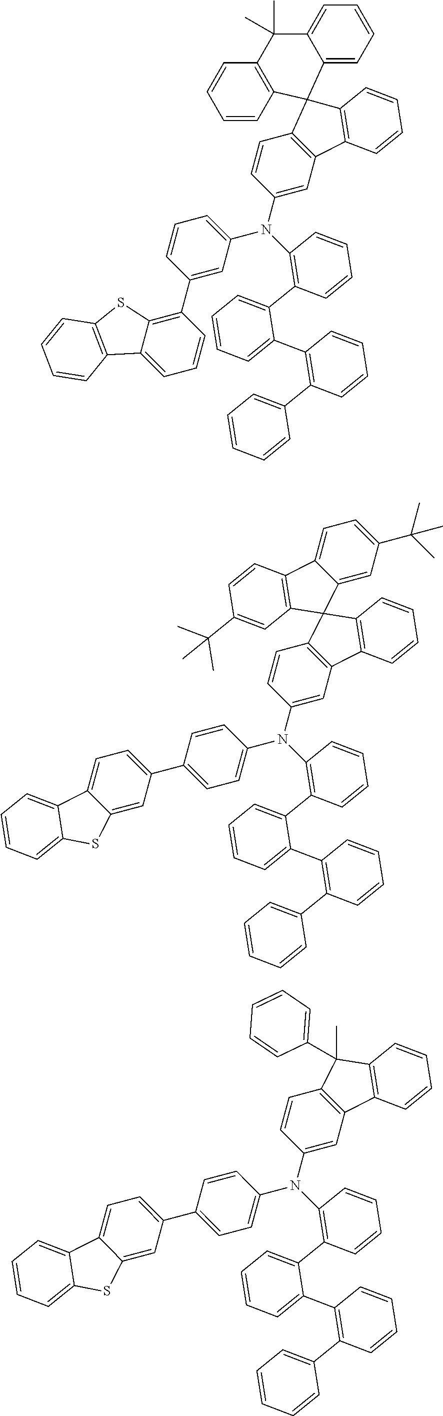

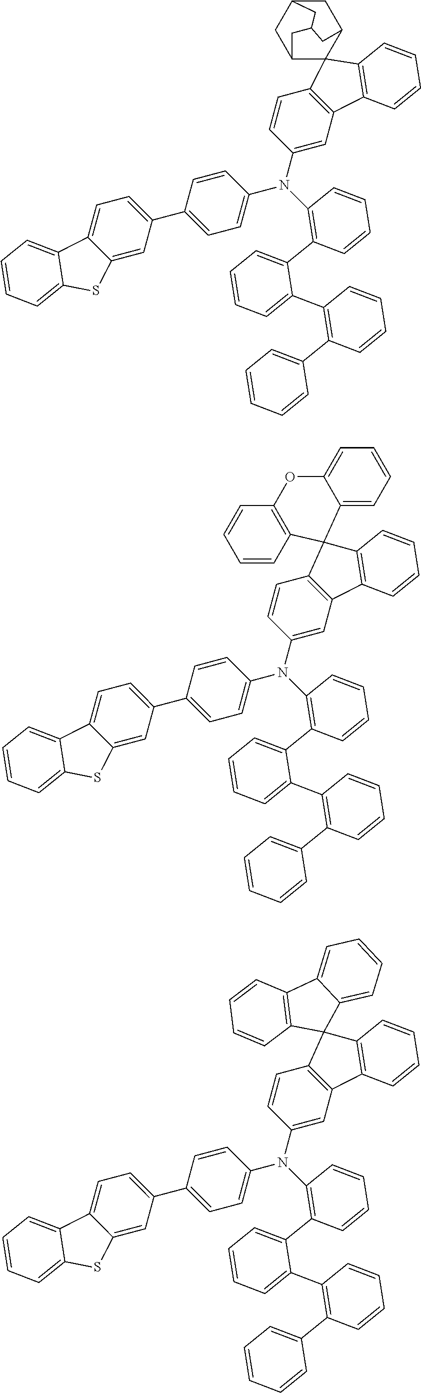 [SMILES: c1ccc(-c2ccccc2-c2ccccc2-c2ccccc2N(c2ccc(-c3ccc4c(c3)sc3ccccc34)cc2)c2ccc3c(c2)-c2ccccc2C32C3CC4CC(C3)CC2C4)cc1 ; c1ccc(-c2ccccc2-c2ccccc2-c2ccccc2N(c2ccc(-c3ccc4c(c3)sc3ccccc34)cc2)c2ccc3c(c2)-c2ccccc2C32c3ccccc3Oc3ccccc32)cc1 ; c1ccc(-c2ccccc2-c2ccccc2-c2ccccc2N(c2ccc(-c3ccc4c(c3)sc3ccccc34)cc2)c2ccc3c(c2)-c2ccccc2C32c3ccccc3-c3ccccc32)cc1]

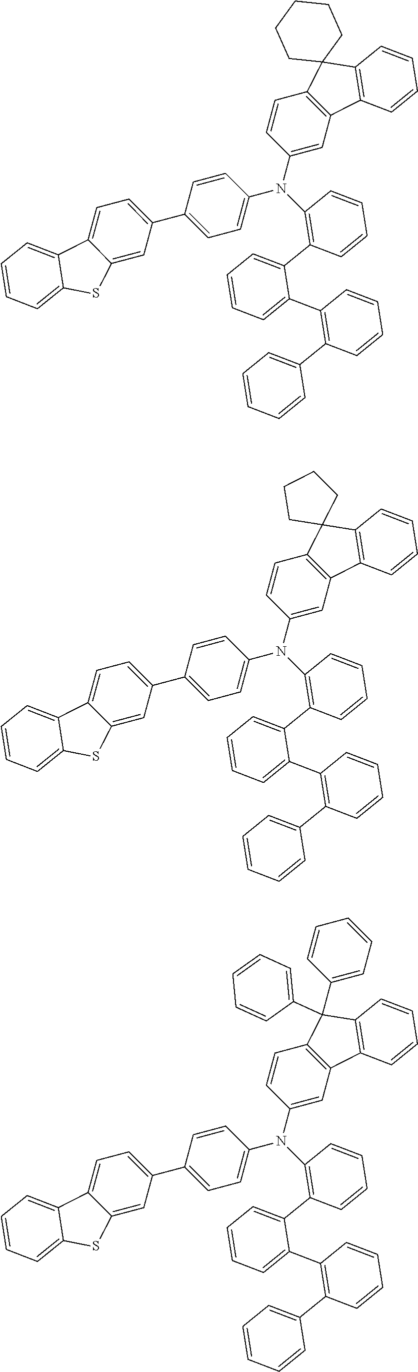 [SMILES: c1ccc(-c2ccccc2-c2ccccc2-c2ccccc2N(c2ccc(-c3ccc4c(c3)sc3ccccc34)cc2)c2ccc3c(c2)-c2ccccc2C32CCCCC2)cc1 ; c1ccc(-c2ccccc2-c2ccccc2-c2ccccc2N(c2ccc(-c3ccc4c(c3)sc3ccccc34)cc2)c2ccc3c(c2)-c2ccccc2C32CCCC2)cc1 ; c1ccc(-c2ccccc2-c2ccccc2-c2ccccc2N(c2ccc(-c3ccc4c(c3)sc3ccccc34)cc2)c2ccc3c(c2)-c2ccccc2C3(c2ccccc2)c2ccccc2)cc1]

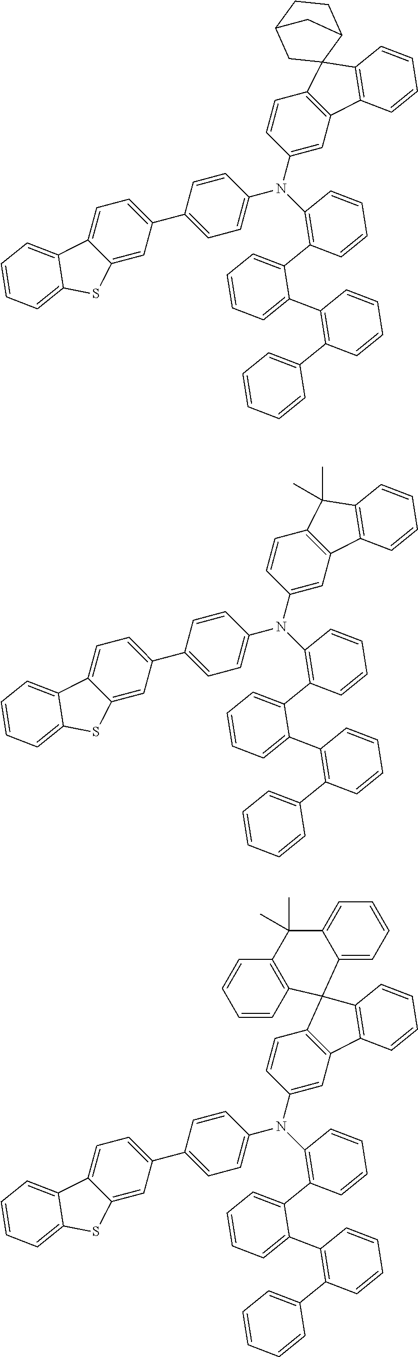

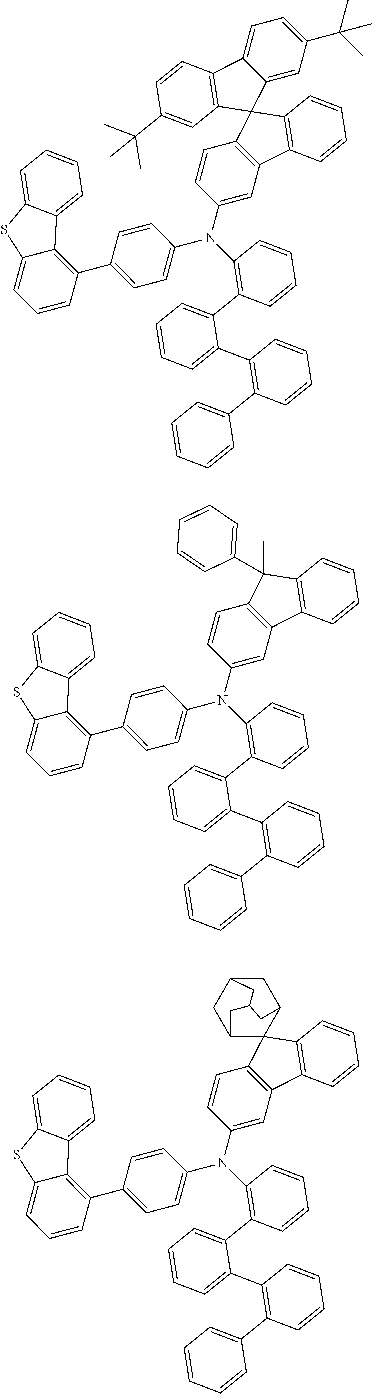

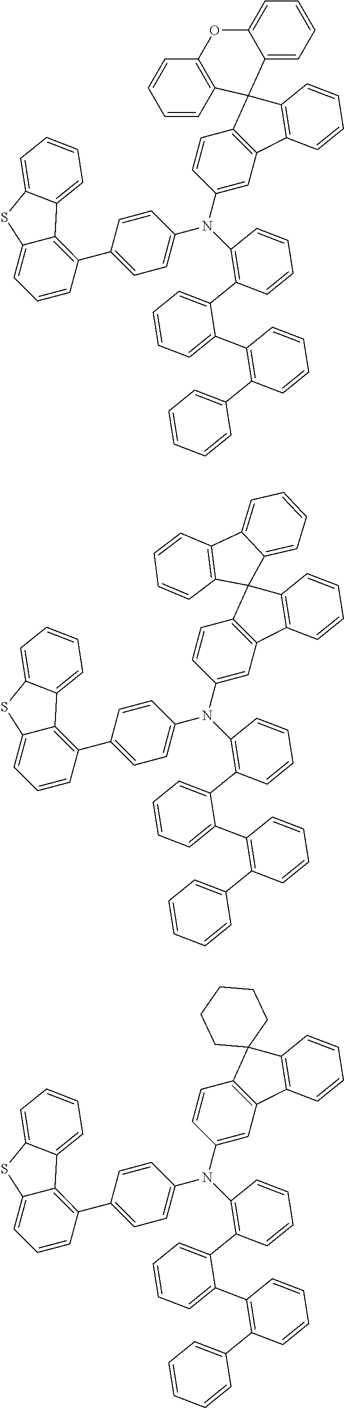

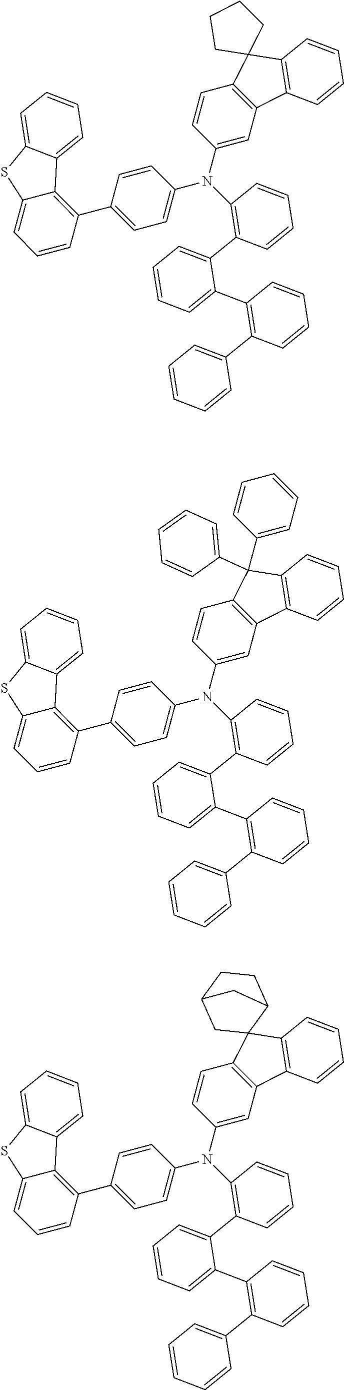 [SMILES: c1ccc(-c2ccccc2-c2ccccc2-c2ccccc2N(c2ccc(-c3cccc4sc5ccccc5c34)cc2)c2ccc3c(c2)-c2ccccc2C32CCCC2)cc1 ; c1ccc(-c2ccccc2-c2ccccc2-c2ccccc2N(c2ccc(-c3cccc4sc5ccccc5c34)cc2)c2ccc3c(c2)-c2ccccc2C3(c2ccccc2)c2ccccc2)cc1 ; c1ccc(-c2ccccc2-c2ccccc2-c2ccccc2N(c2ccc(-c3cccc4sc5ccccc5c34)cc2)c2ccc3c(c2)-c2ccccc2C32CC3CCC2C3)cc1]

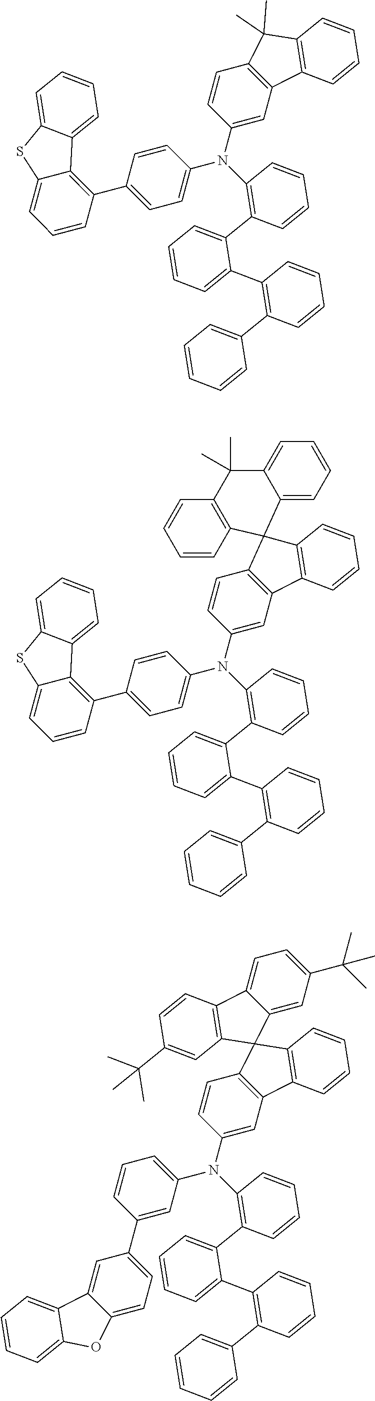 [SMILES: CC1(C)c2ccccc2-c2cc(N(c3ccc(-c4cccc5sc6ccccc6c45)cc3)c3ccccc3-c3ccccc3-c3ccccc3-c3ccccc3)ccc21 ; CC1(C)c2ccccc2C2(c3ccccc3-c3cc(N(c4ccc(-c5cccc6sc7ccccc7c56)cc4)c4ccccc4-c4ccccc4-c4ccccc4-c4ccccc4)ccc32)c2ccccc21 ; CC(C)(C)c1ccc2c(c1)C1(c3ccccc3-c3cc(N(c4cccc(-c5ccc6oc7ccccc7c6c5)c4)c4ccccc4-c4ccccc4-c4ccccc4-c4ccccc4)ccc31)c1cc(C(C)(C)C)ccc1-2]

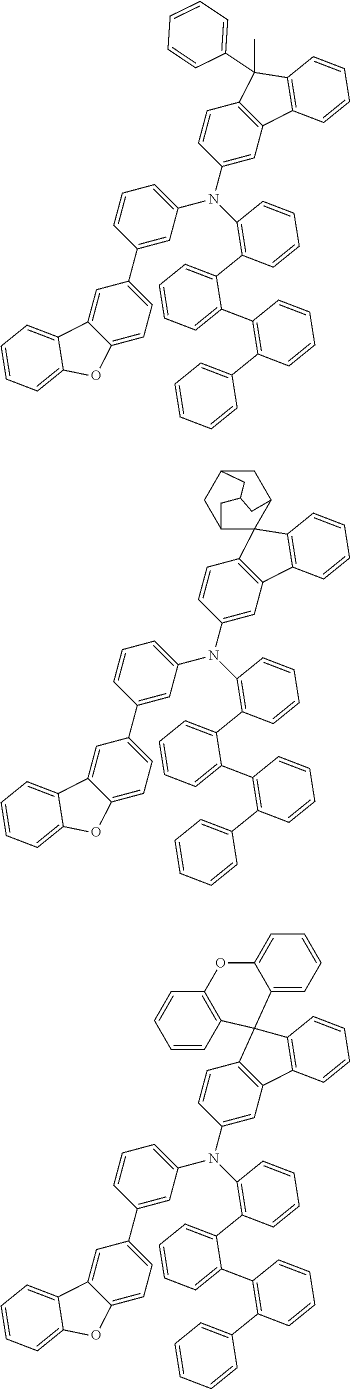

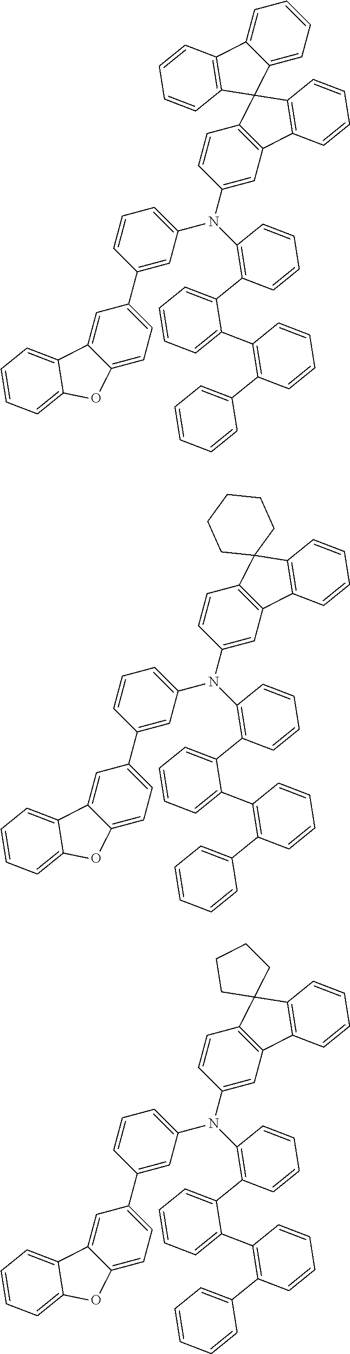

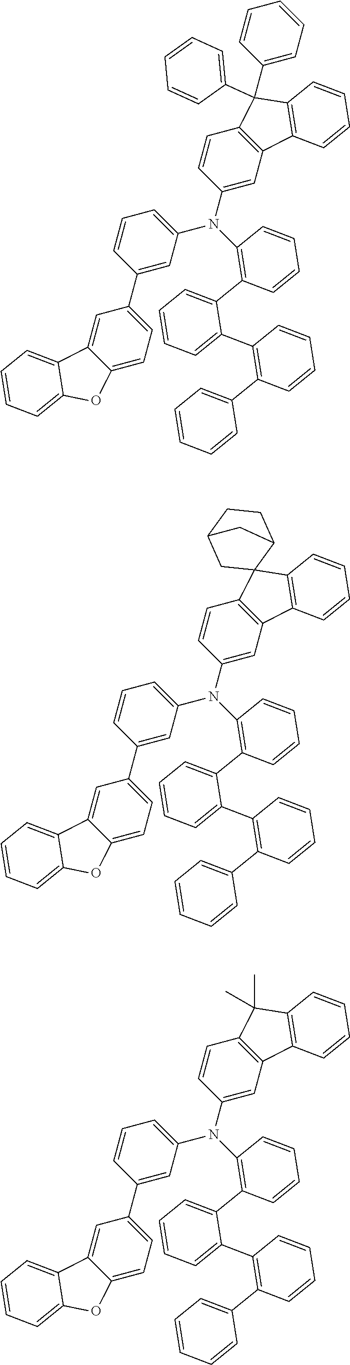 [SMILES: c1ccc(-c2ccccc2-c2ccccc2-c2ccccc2N(c2cccc(-c3ccc4oc5ccccc5c4c3)c2)c2ccc3c(c2)-c2ccccc2C3(c2ccccc2)c2ccccc2)cc1 ; c1ccc(-c2ccccc2-c2ccccc2-c2ccccc2N(c2cccc(-c3ccc4oc5ccccc5c4c3)c2)c2ccc3c(c2)-c2ccccc2C32CC3CCC2C3)cc1 ; CC1(C)c2ccccc2-c2cc(N(c3cccc(-c4ccc5oc6ccccc6c5c4)c3)c3ccccc3-c3ccccc3-c3ccccc3-c3ccccc3)ccc21]

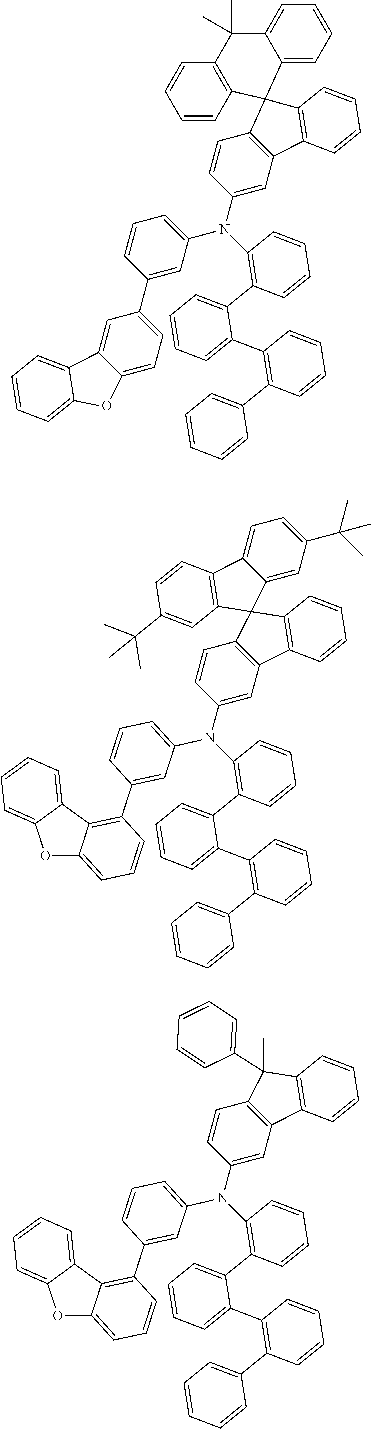

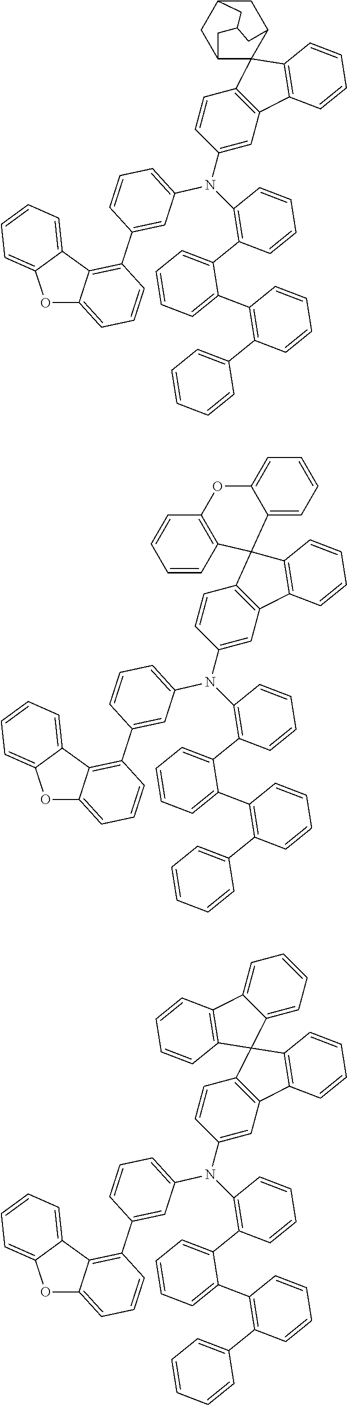 [SMILES: c1ccc(-c2ccccc2-c2ccccc2-c2ccccc2N(c2cccc(-c3cccc4oc5ccccc5c34)c2)c2ccc3c(c2)-c2ccccc2C32C3CC4CC(C3)CC2C4)cc1 ; c1ccc(-c2ccccc2-c2ccccc2-c2ccccc2N(c2cccc(-c3cccc4oc5ccccc5c34)c2)c2ccc3c(c2)-c2ccccc2C32c3ccccc3Oc3ccccc32)cc1 ; c1ccc(-c2ccccc2-c2ccccc2-c2ccccc2N(c2cccc(-c3cccc4oc5ccccc5c34)c2)c2ccc3c(c2)-c2ccccc2C32c3ccccc3-c3ccccc32)cc1]

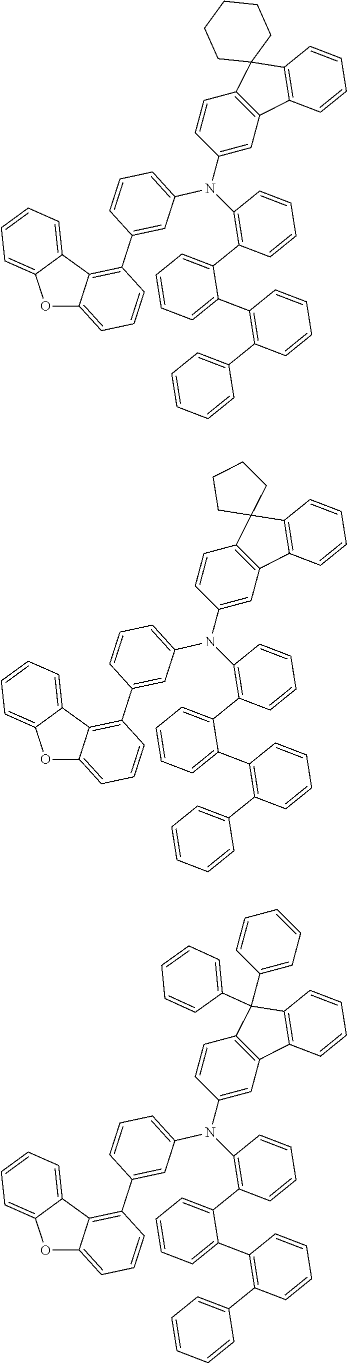 [SMILES: c1ccc(-c2ccccc2-c2ccccc2-c2ccccc2N(c2cccc(-c3cccc4oc5ccccc5c34)c2)c2ccc3c(c2)-c2ccccc2C32CCCCC2)cc1 ; c1ccc(-c2ccccc2-c2ccccc2-c2ccccc2N(c2cccc(-c3cccc4oc5ccccc5c34)c2)c2ccc3c(c2)-c2ccccc2C32CCCC2)cc1 ; c1ccc(-c2ccccc2-c2ccccc2-c2ccccc2N(c2cccc(-c3cccc4oc5ccccc5c34)c2)c2ccc3c(c2)-c2ccccc2C3(c2ccccc2)c2ccccc2)cc1]

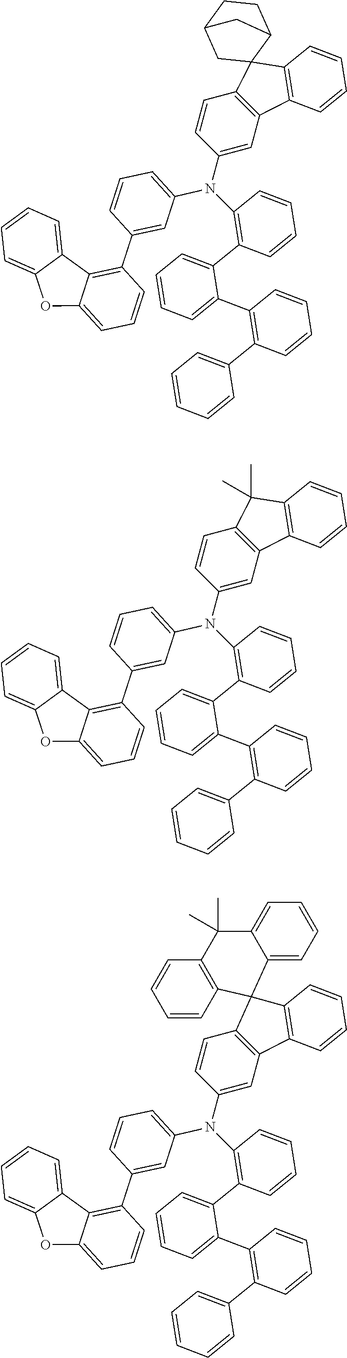

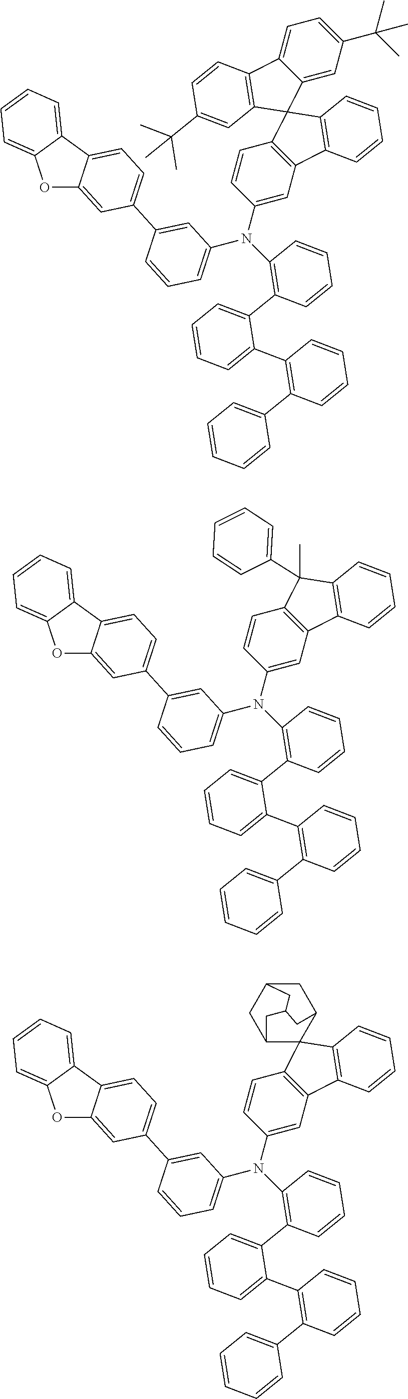

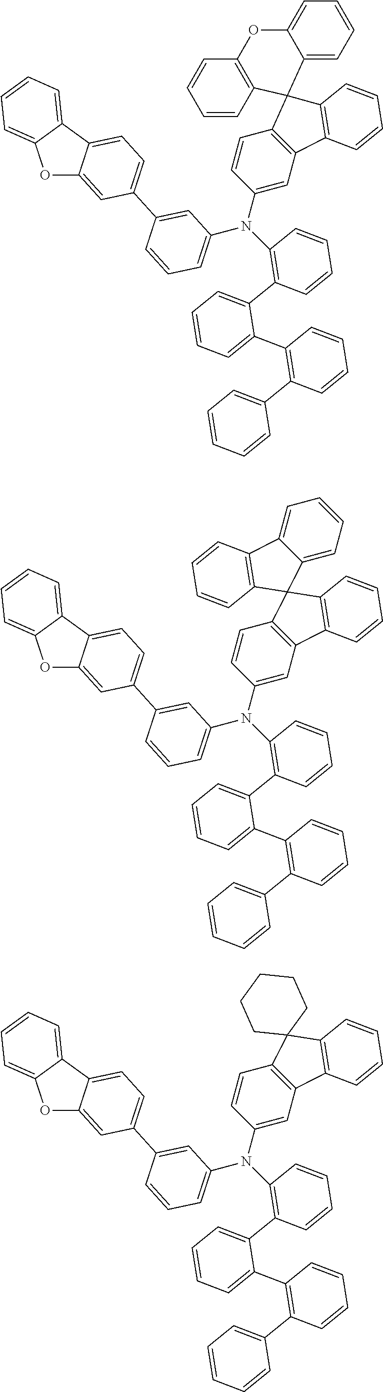

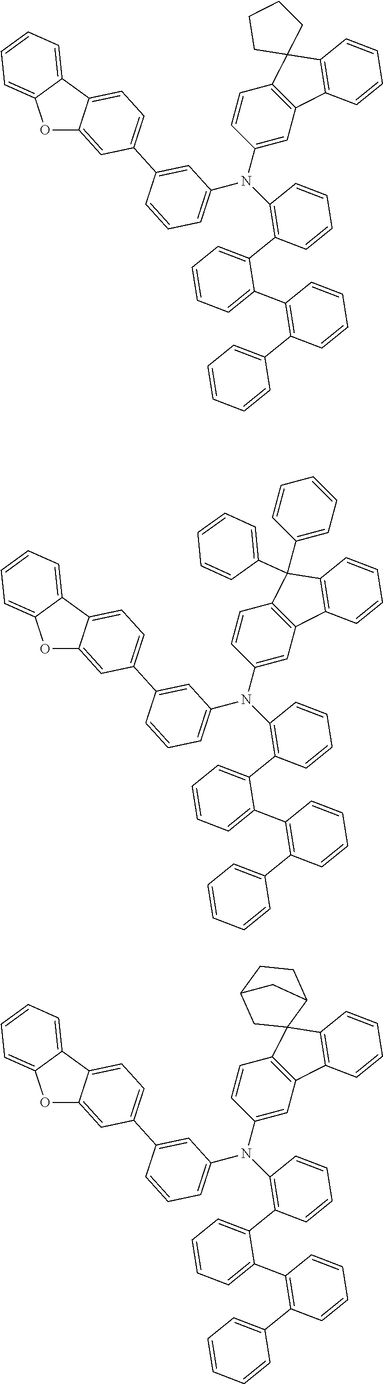

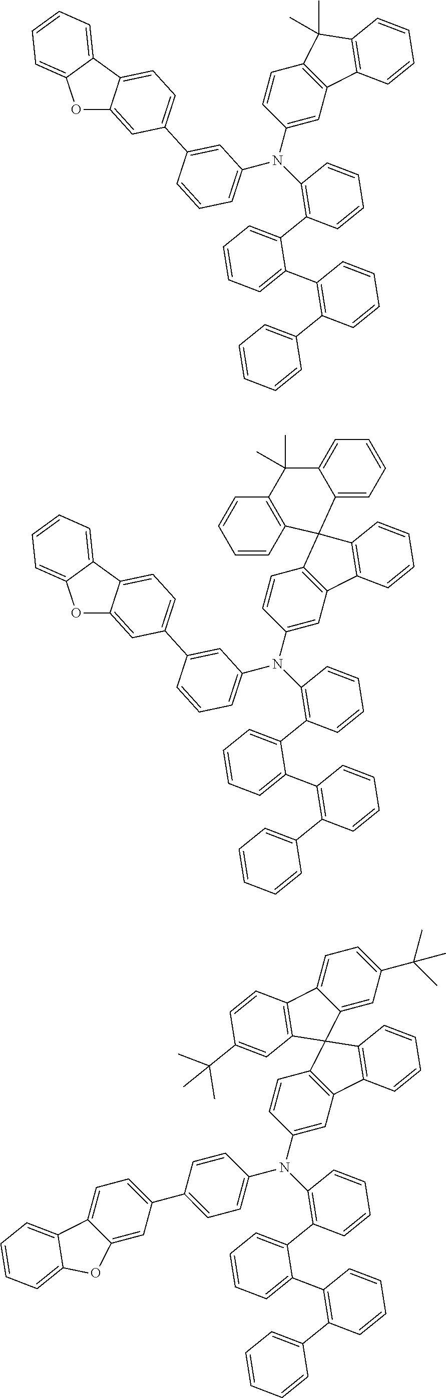

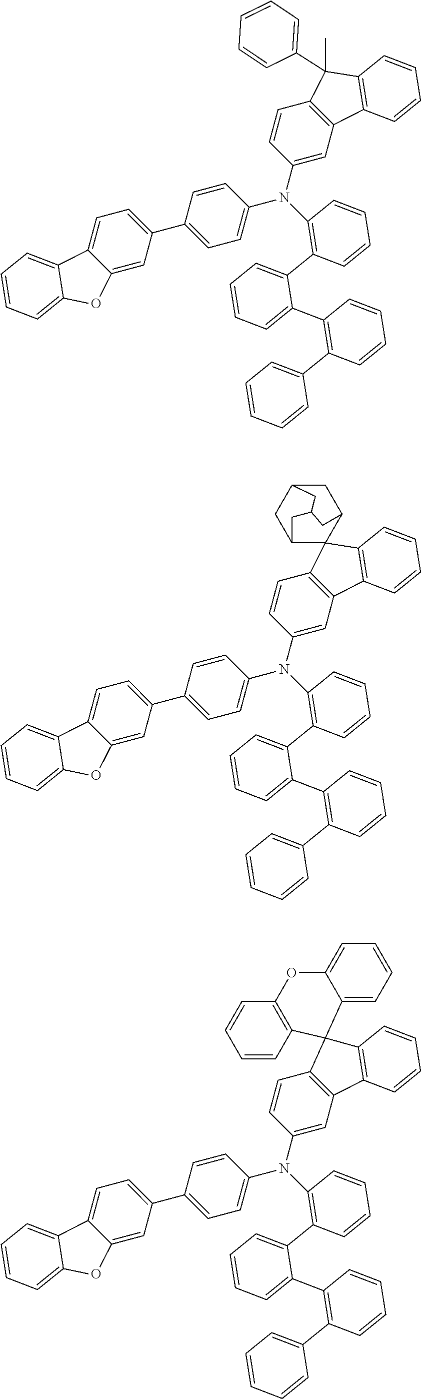 [SMILES: CC1(c2ccccc2)c2ccccc2-c2cc(N(c3ccc(-c4ccc5c(c4)oc4ccccc45)cc3)c3ccccc3-c3ccccc3-c3ccccc3-c3ccccc3)ccc21 ; c1ccc(-c2ccccc2-c2ccccc2-c2ccccc2N(c2ccc(-c3ccc4c(c3)oc3ccccc34)cc2)c2ccc3c(c2)-c2ccccc2C32C3CC4CC(C3)CC2C4)cc1 ; c1ccc(-c2ccccc2-c2ccccc2-c2ccccc2N(c2ccc(-c3ccc4c(c3)oc3ccccc34)cc2)c2ccc3c(c2)-c2ccccc2C32c3ccccc3Oc3ccccc32)cc1]

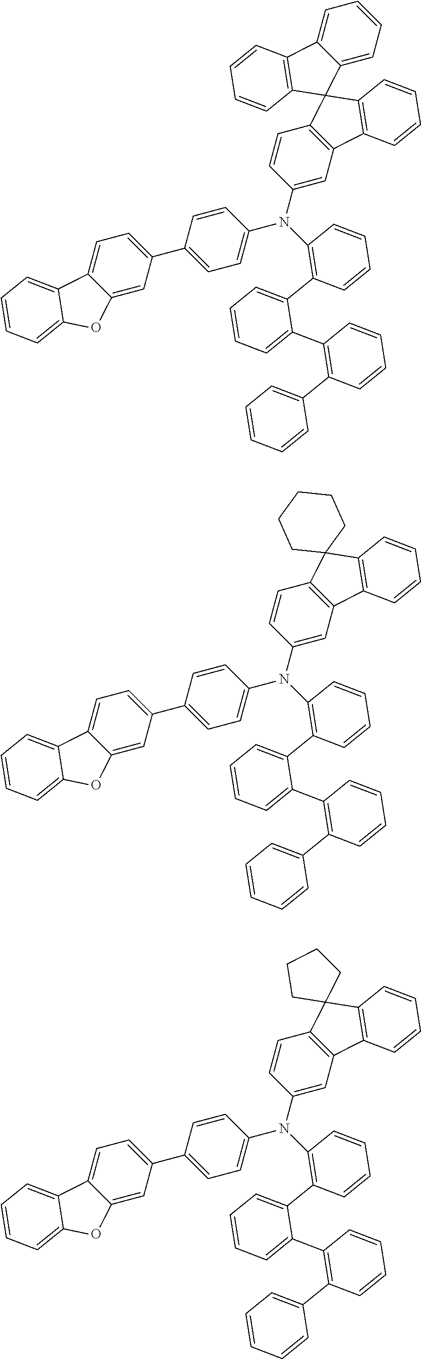

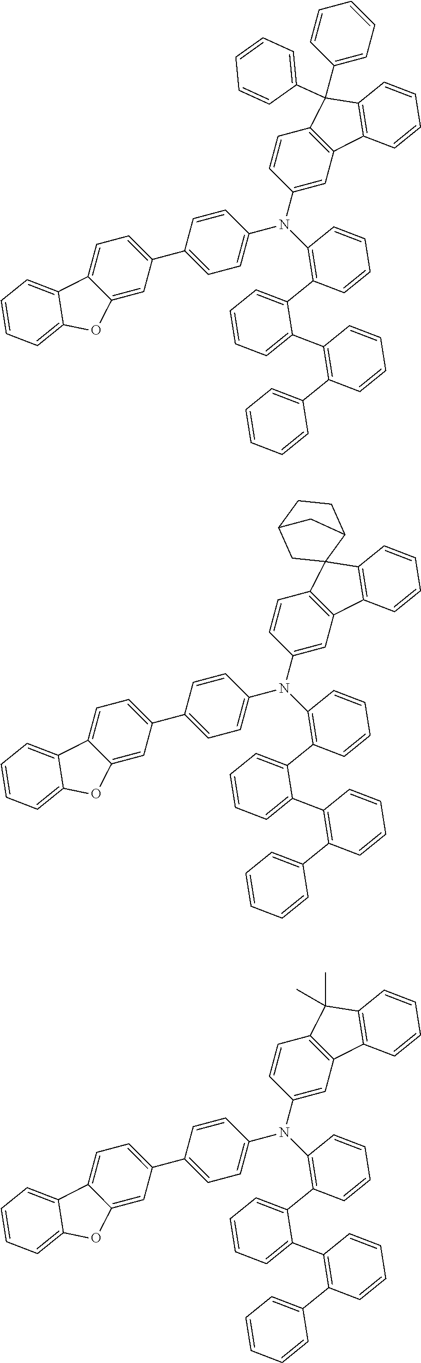 [SMILES: c1ccc(-c2ccccc2-c2ccccc2-c2ccccc2N(c2ccc(-c3ccc4c(c3)oc3ccccc34)cc2)c2ccc3c(c2)-c2ccccc2C3(c2ccccc2)c2ccccc2)cc1 ; c1ccc(-c2ccccc2-c2ccccc2-c2ccccc2N(c2ccc(-c3ccc4c(c3)oc3ccccc34)cc2)c2ccc3c(c2)-c2ccccc2C32CC3CCC2C3)cc1 ; CC1(C)c2ccccc2-c2cc(N(c3ccc(-c4ccc5c(c4)oc4ccccc45)cc3)c3ccccc3-c3ccccc3-c3ccccc3-c3ccccc3)ccc21]

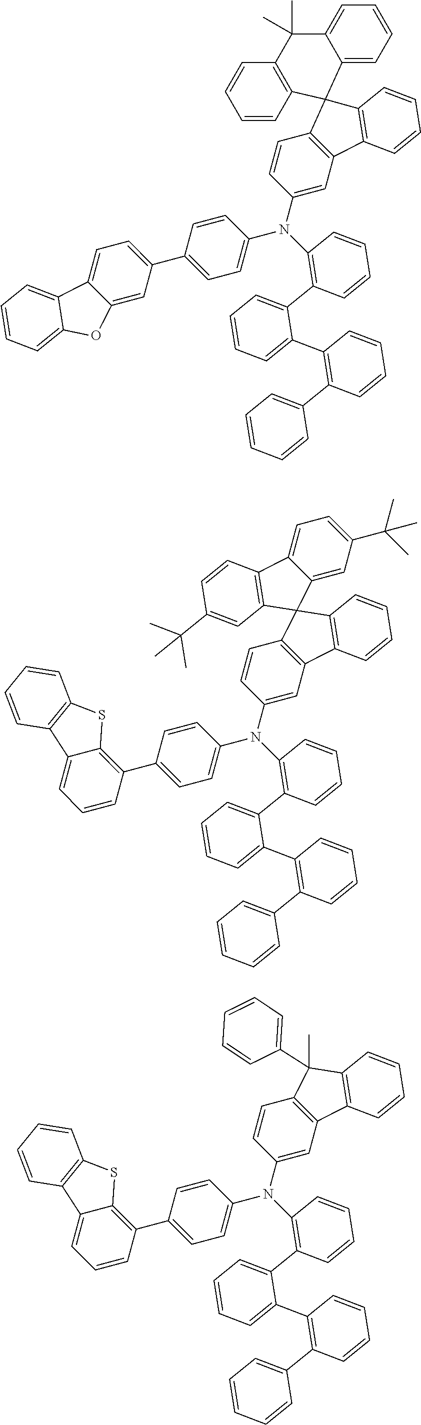

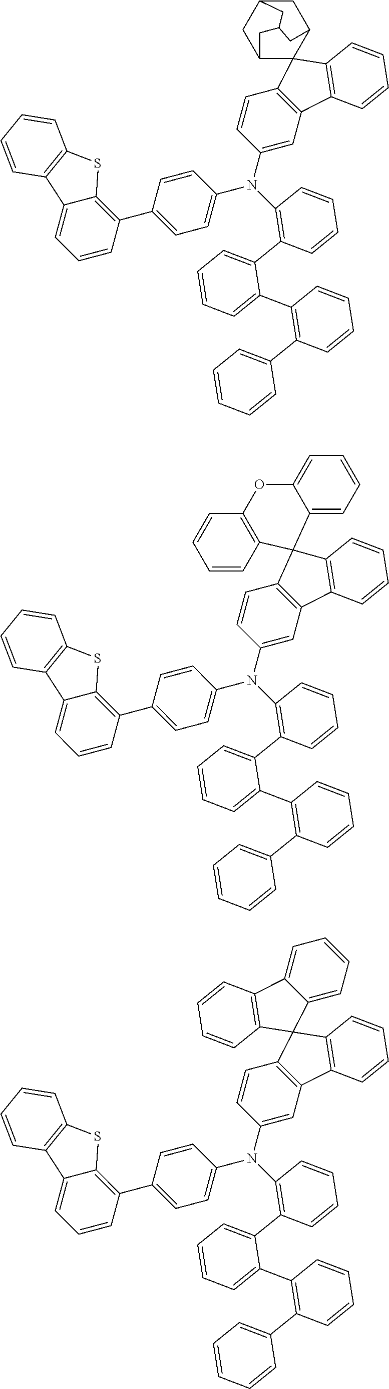 [SMILES: c1ccc(-c2ccccc2-c2ccccc2-c2ccccc2N(c2ccc(-c3cccc4c3sc3ccccc34)cc2)c2ccc3c(c2)-c2ccccc2C32C3CC4CC(C3)CC2C4)cc1 ; c1ccc(-c2ccccc2-c2ccccc2-c2ccccc2N(c2ccc(-c3cccc4c3sc3ccccc34)cc2)c2ccc3c(c2)-c2ccccc2C32c3ccccc3Oc3ccccc32)cc1 ; c1ccc(-c2ccccc2-c2ccccc2-c2ccccc2N(c2ccc(-c3cccc4c3sc3ccccc34)cc2)c2ccc3c(c2)-c2ccccc2C32c3ccccc3-c3ccccc32)cc1]

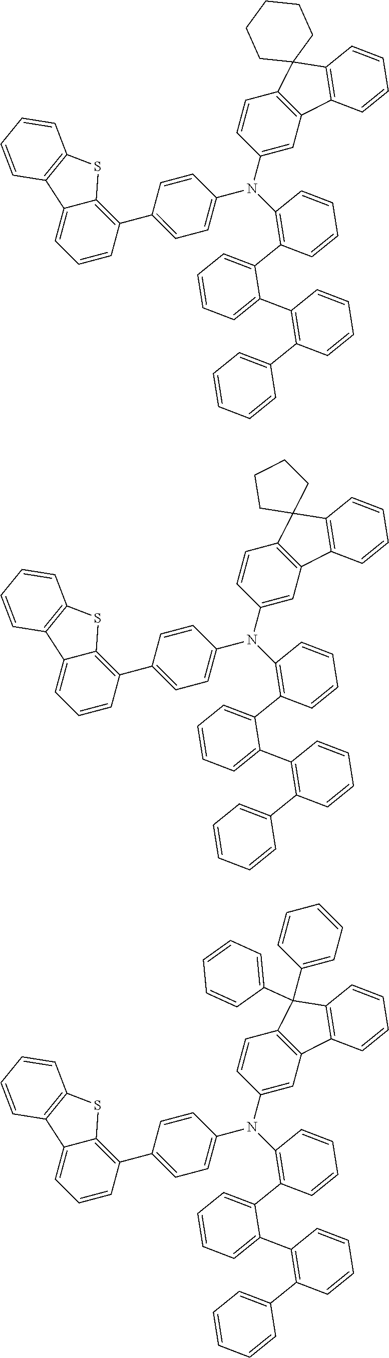 [SMILES: c1ccc(-c2ccccc2-c2ccccc2-c2ccccc2N(c2ccc(-c3cccc4c3sc3ccccc34)cc2)c2ccc3c(c2)-c2ccccc2C32CCCCC2)cc1 ; c1ccc(-c2ccccc2-c2ccccc2-c2ccccc2N(c2ccc(-c3cccc4c3sc3ccccc34)cc2)c2ccc3c(c2)-c2ccccc2C32CCCC2)cc1 ; c1ccc(-c2ccccc2-c2ccccc2-c2ccccc2N(c2ccc(-c3cccc4c3sc3ccccc34)cc2)c2ccc3c(c2)-c2ccccc2C3(c2ccccc2)c2ccccc2)cc1]

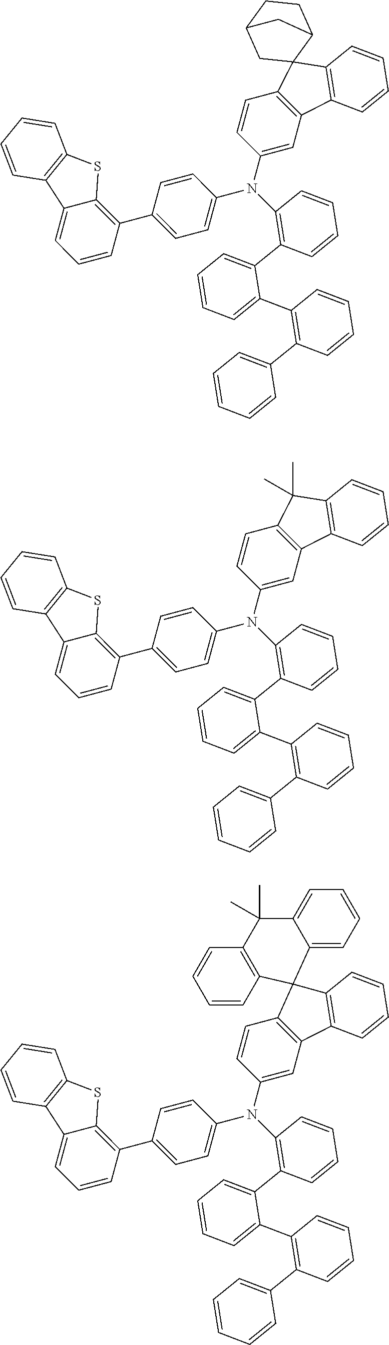

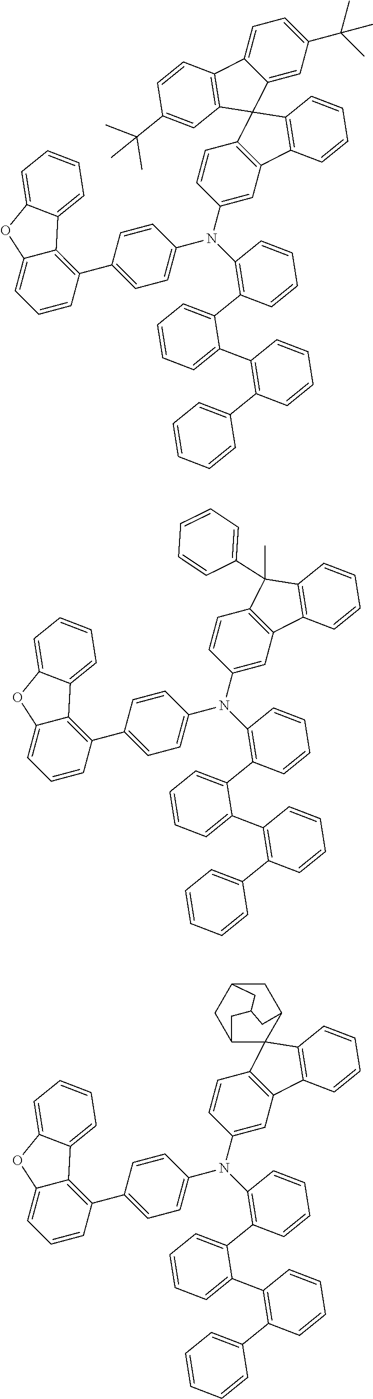

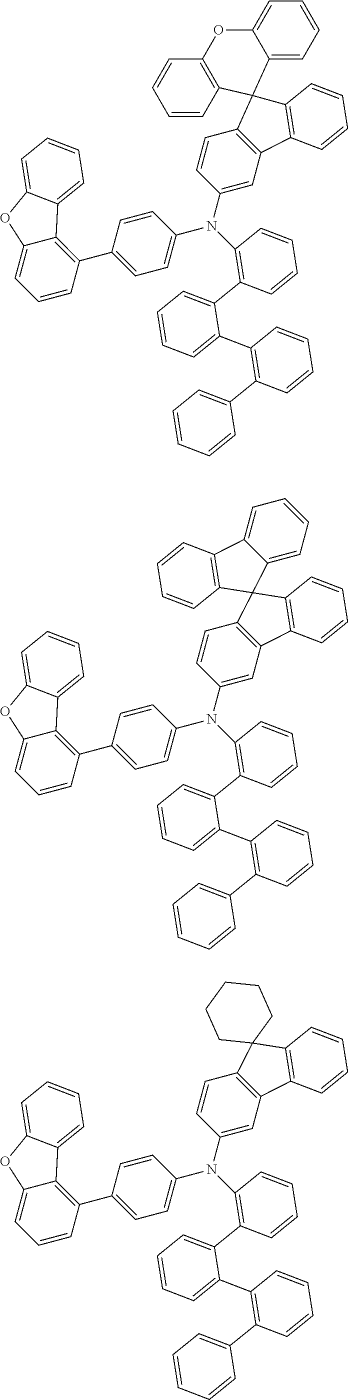

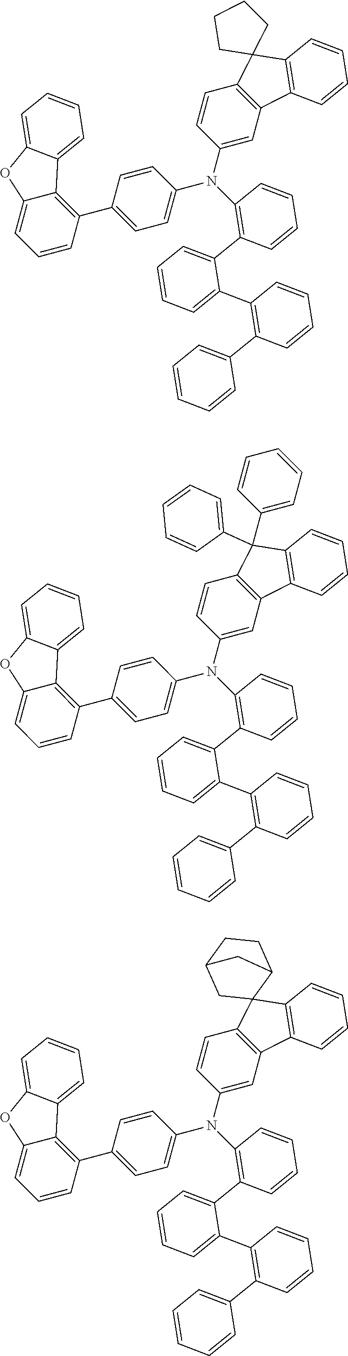

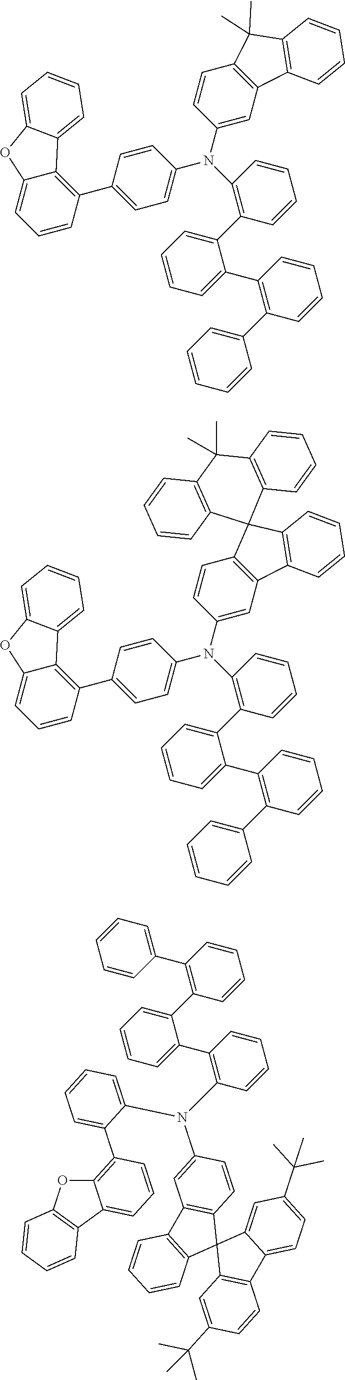

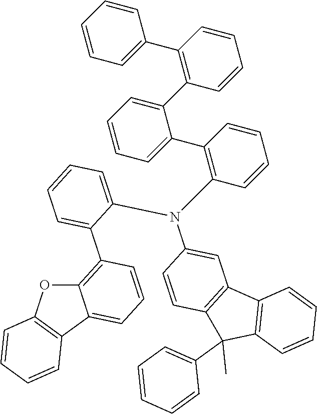 [SMILES: CC1(c2ccccc2)c2ccccc2-c2cc(N(c3ccccc3-c3ccccc3-c3ccccc3-c3ccccc3)c3ccccc3-c3cccc4c3oc3ccccc34)ccc21]

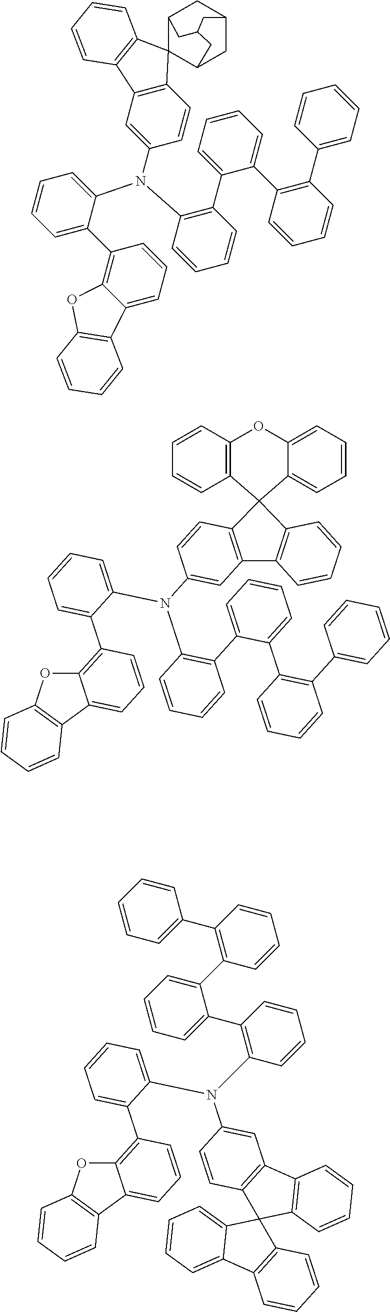 [SMILES: c1ccc(-c2ccccc2-c2ccccc2-c2ccccc2N(c2ccc3c(c2)-c2ccccc2C32C3CC4CC(C3)CC2C4)c2ccccc2-c2cccc3c2oc2ccccc23)cc1 ; c1ccc(-c2ccccc2-c2ccccc2-c2ccccc2N(c2ccc3c(c2)-c2ccccc2C32c3ccccc3Oc3ccccc32)c2ccccc2-c2cccc3c2oc2ccccc23)cc1 ; c1ccc(-c2ccccc2-c2ccccc2-c2ccccc2N(c2ccc3c(c2)-c2ccccc2C32c3ccccc3-c3ccccc32)c2ccccc2-c2cccc3c2oc2ccccc23)cc1]

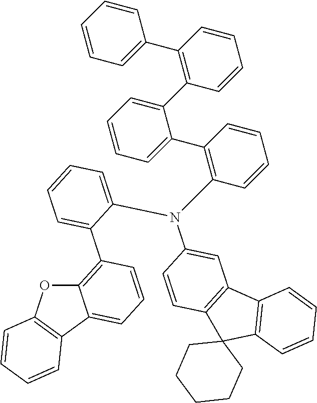 [SMILES: c1ccc(-c2ccccc2-c2ccccc2-c2ccccc2N(c2ccc3c(c2)-c2ccccc2C32CCCCC2)c2ccccc2-c2cccc3c2oc2ccccc23)cc1]

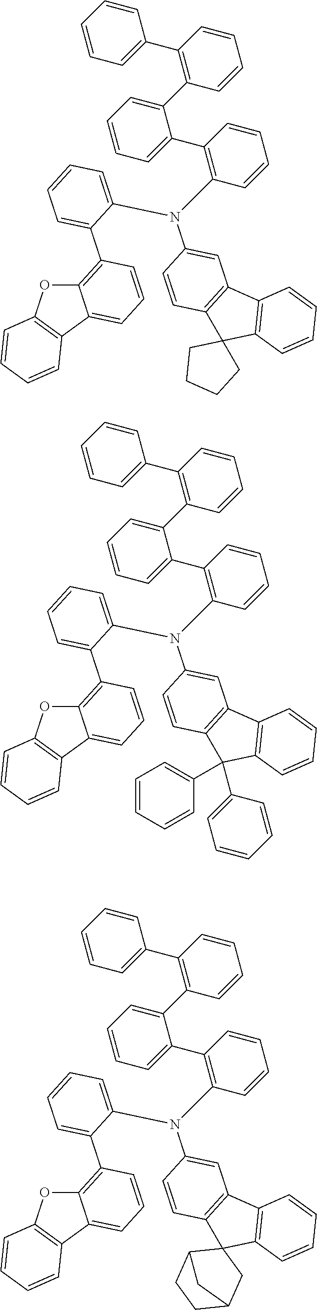 [SMILES: c1ccc(-c2ccccc2-c2ccccc2-c2ccccc2N(c2ccc3c(c2)-c2ccccc2C32CCCC2)c2ccccc2-c2cccc3c2oc2ccccc23)cc1 ; c1ccc(-c2ccccc2-c2ccccc2-c2ccccc2N(c2ccc3c(c2)-c2ccccc2C3(c2ccccc2)c2ccccc2)c2ccccc2-c2cccc3c2oc2ccccc23)cc1 ; c1ccc(-c2ccccc2-c2ccccc2-c2ccccc2N(c2ccc3c(c2)-c2ccccc2C32CC3CCC2C3)c2ccccc2-c2cccc3c2oc2ccccc23)cc1]

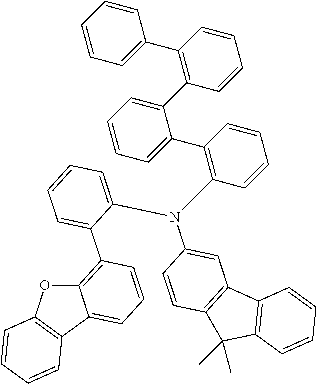 [SMILES: CC1(C)c2ccccc2-c2cc(N(c3ccccc3-c3ccccc3-c3ccccc3-c3ccccc3)c3ccccc3-c3cccc4c3oc3ccccc34)ccc21]

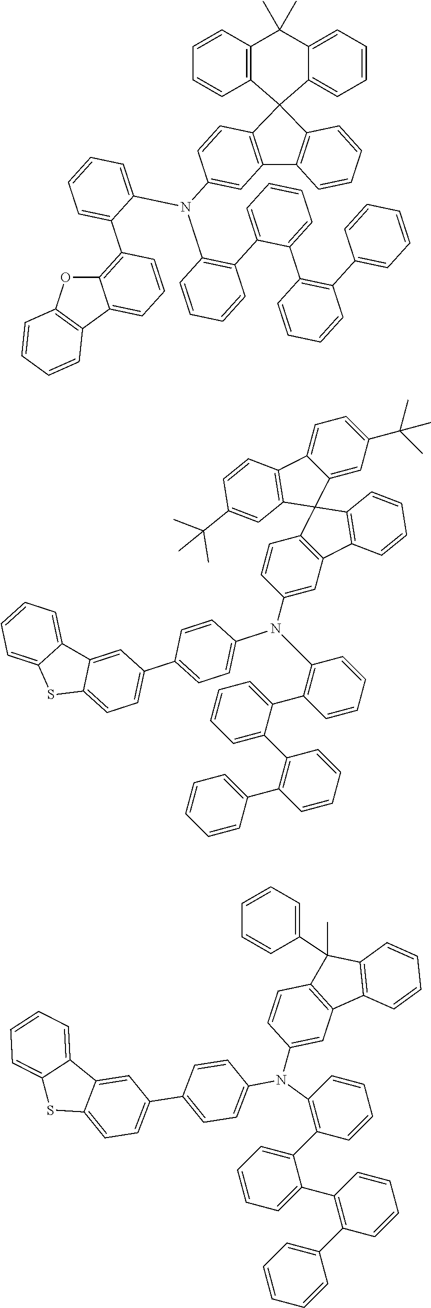 [SMILES: CC1(C)c2ccccc2C2(c3ccccc3-c3cc(N(c4ccccc4-c4ccccc4-c4ccccc4-c4ccccc4)c4ccccc4-c4cccc5c4oc4ccccc45)ccc32)c2ccccc21 ; CC(C)(C)c1ccc2c(c1)C1(c3ccccc3-c3cc(N(c4ccc(-c5ccc6sc7ccccc7c6c5)cc4)c4ccccc4-c4ccccc4-c4ccccc4-c4ccccc4)ccc31)c1cc(C(C)(C)C)ccc1-2 ; CC1(c2ccccc2)c2ccccc2-c2cc(N(c3ccc(-c4ccc5sc6ccccc6c5c4)cc3)c3ccccc3-c3ccccc3-c3ccccc3-c3ccccc3)ccc21]

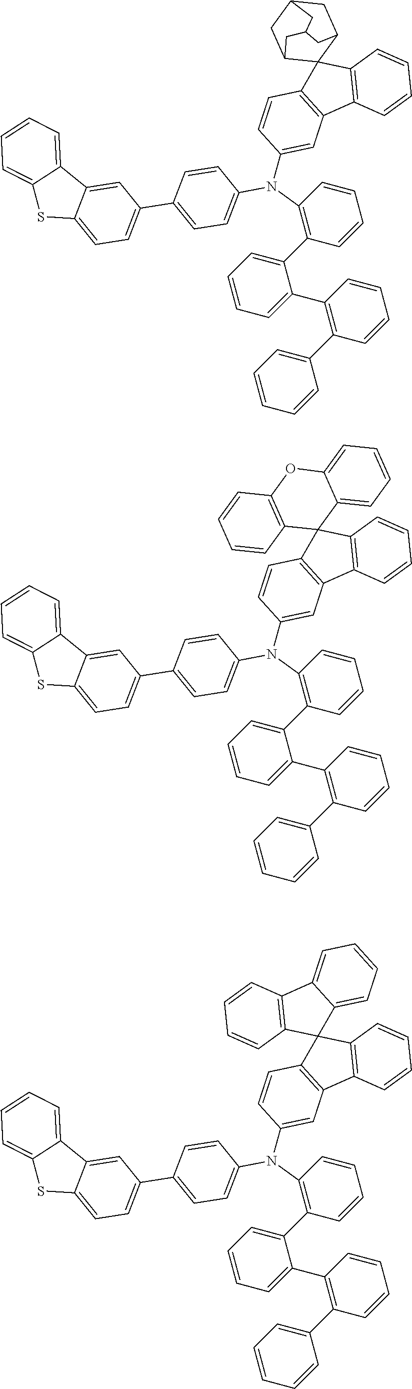 [SMILES: c1ccc(-c2ccccc2-c2ccccc2-c2ccccc2N(c2ccc(-c3ccc4sc5ccccc5c4c3)cc2)c2ccc3c(c2)-c2ccccc2C32C3CC4CC(C3)CC2C4)cc1 ; c1ccc(-c2ccccc2-c2ccccc2-c2ccccc2N(c2ccc(-c3ccc4sc5ccccc5c4c3)cc2)c2ccc3c(c2)-c2ccccc2C32c3ccccc3Oc3ccccc32)cc1 ; c1ccc(-c2ccccc2-c2ccccc2-c2ccccc2N(c2ccc(-c3ccc4sc5ccccc5c4c3)cc2)c2ccc3c(c2)-c2ccccc2C32c3ccccc3-c3ccccc32)cc1]

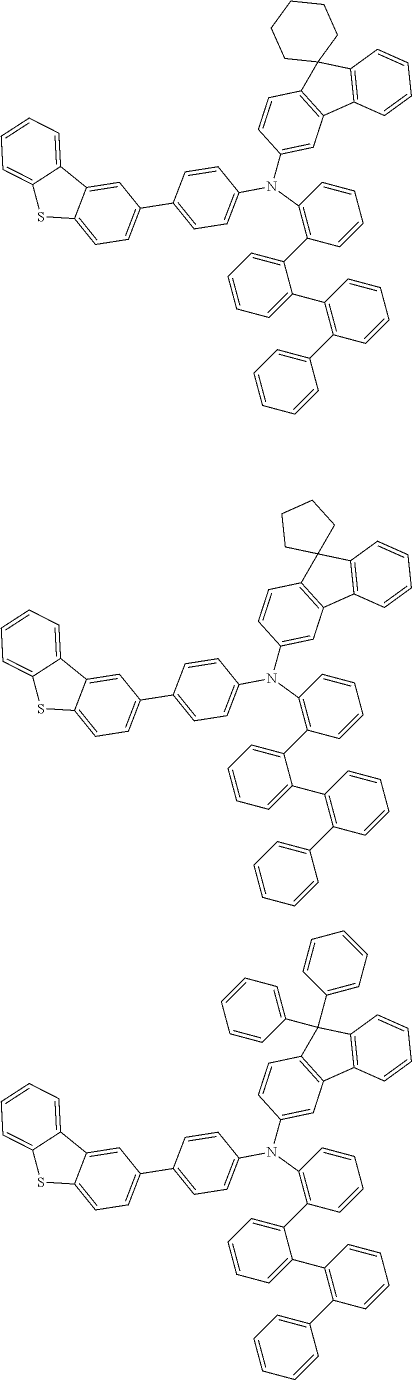

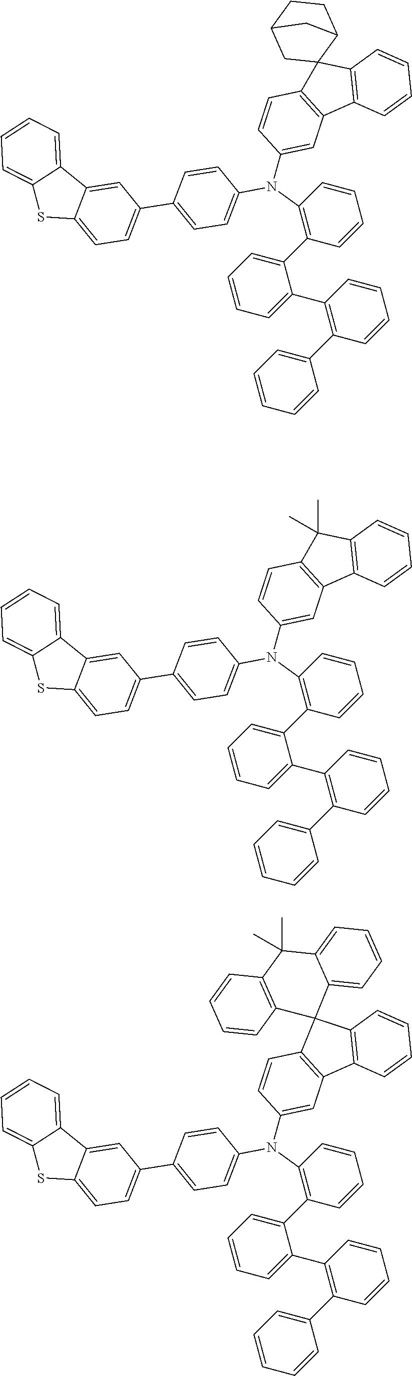

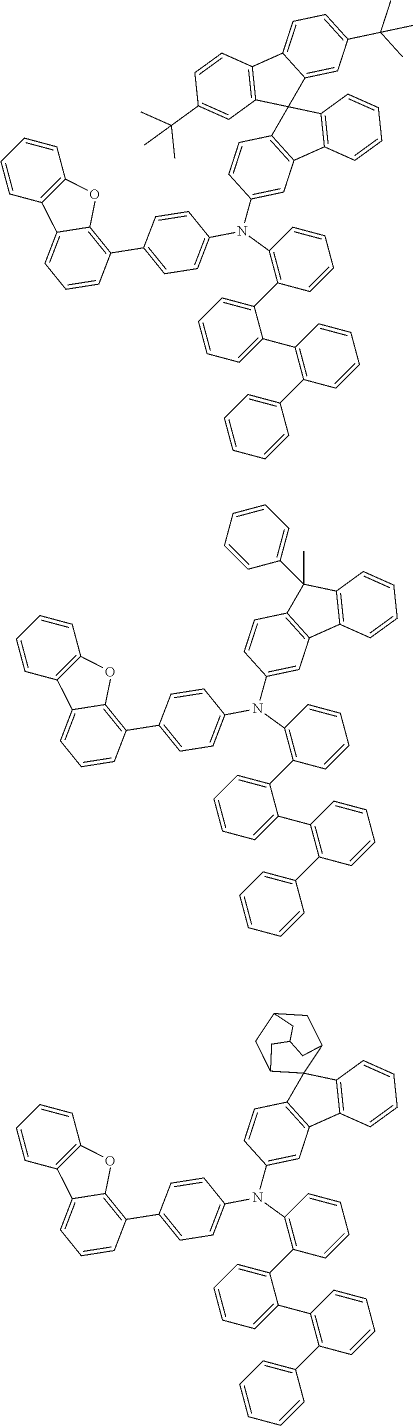

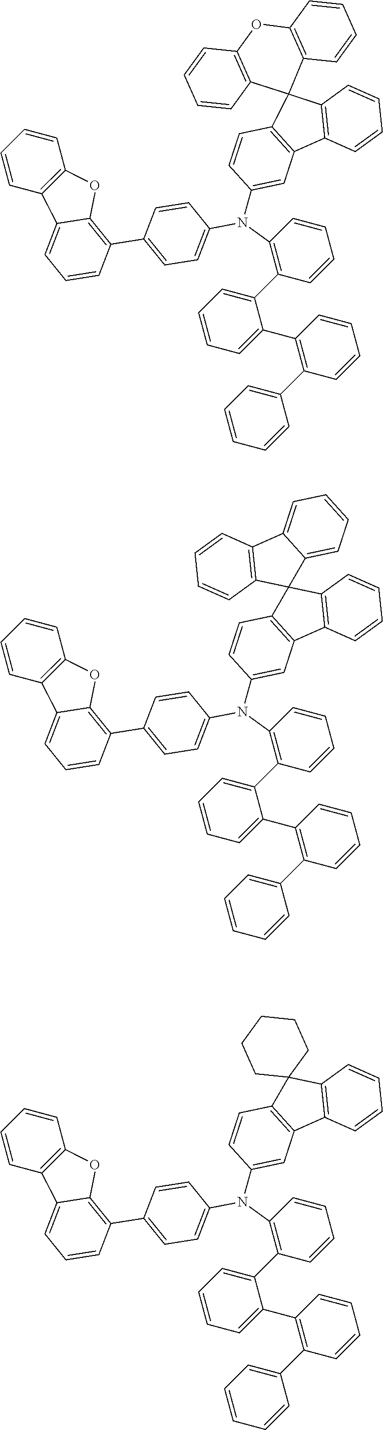 [SMILES: c1ccc(-c2ccccc2-c2ccccc2-c2ccccc2N(c2ccc(-c3cccc4c3oc3ccccc34)cc2)c2ccc3c(c2)-c2ccccc2C32c3ccccc3Oc3ccccc32)cc1 ; c1ccc(-c2ccccc2-c2ccccc2-c2ccccc2N(c2ccc(-c3cccc4c3oc3ccccc34)cc2)c2ccc3c(c2)-c2ccccc2C32c3ccccc3-c3ccccc32)cc1 ; c1ccc(-c2ccccc2-c2ccccc2-c2ccccc2N(c2ccc(-c3cccc4c3oc3ccccc34)cc2)c2ccc3c(c2)-c2ccccc2C32CCCCC2)cc1]

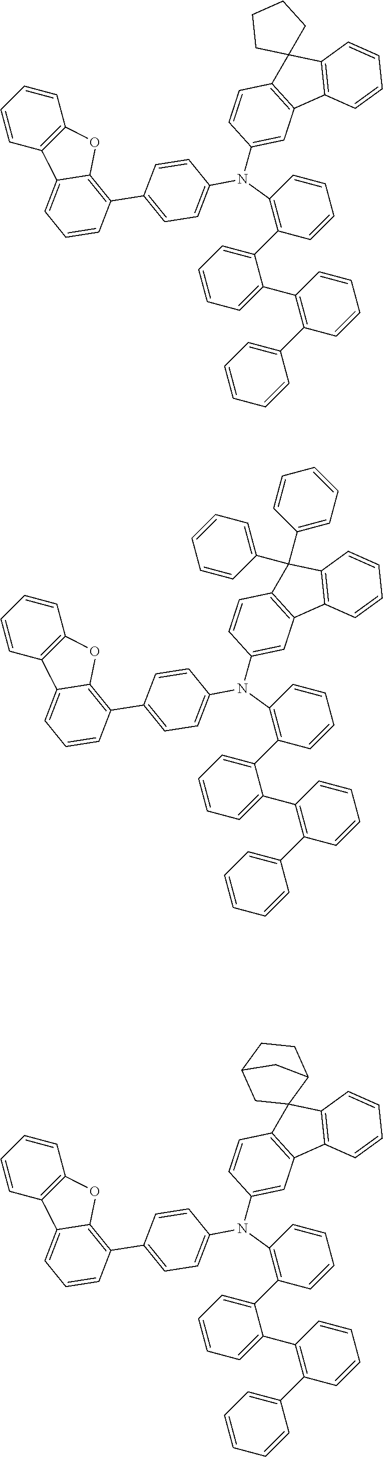 [SMILES: c1ccc(-c2ccccc2-c2ccccc2-c2ccccc2N(c2ccc(-c3cccc4c3oc3ccccc34)cc2)c2ccc3c(c2)-c2ccccc2C32CCCC2)cc1 ; c1ccc(-c2ccccc2-c2ccccc2-c2ccccc2N(c2ccc(-c3cccc4c3oc3ccccc34)cc2)c2ccc3c(c2)-c2ccccc2C3(c2ccccc2)c2ccccc2)cc1 ; c1ccc(-c2ccccc2-c2ccccc2-c2ccccc2N(c2ccc(-c3cccc4c3oc3ccccc34)cc2)c2ccc3c(c2)-c2ccccc2C32CC3CCC2C3)cc1]

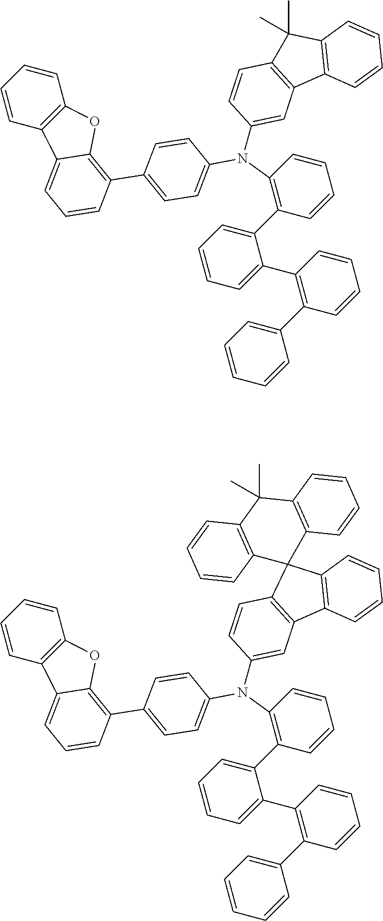 [SMILES: CC1(C)c2ccccc2-c2cc(N(c3ccc(-c4cccc5c4oc4ccccc45)cc3)c3ccccc3-c3ccccc3-c3ccccc3-c3ccccc3)ccc21 ; CC1(C)c2ccccc2C2(c3ccccc3-c3cc(N(c4ccc(-c5cccc6c5oc5ccccc56)cc4)c4ccccc4-c4ccccc4-c4ccccc4-c4ccccc4)ccc32)c2ccccc21]

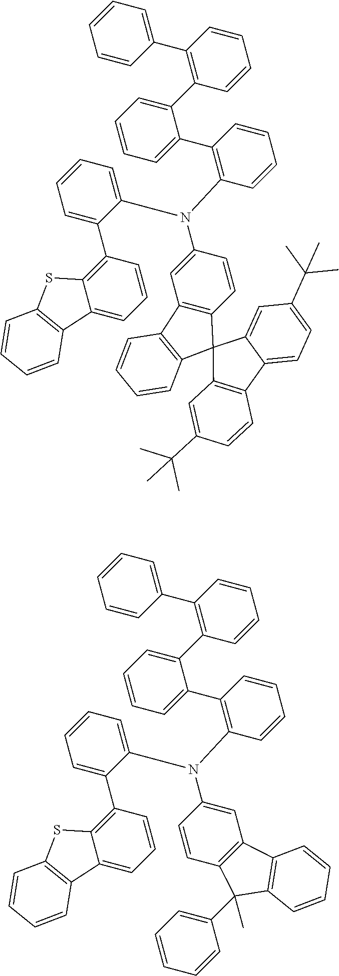 [SMILES: CC(C)(C)c1ccc2c(c1)C1(c3ccccc3-c3cc(N(c4ccccc4-c4ccccc4-c4ccccc4-c4ccccc4)c4ccccc4-c4cccc5c4sc4ccccc45)ccc31)c1cc(C(C)(C)C)ccc1-2 ; CC1(c2ccccc2)c2ccccc2-c2cc(N(c3ccccc3-c3ccccc3-c3ccccc3-c3ccccc3)c3ccccc3-c3cccc4c3sc3ccccc34)ccc21]

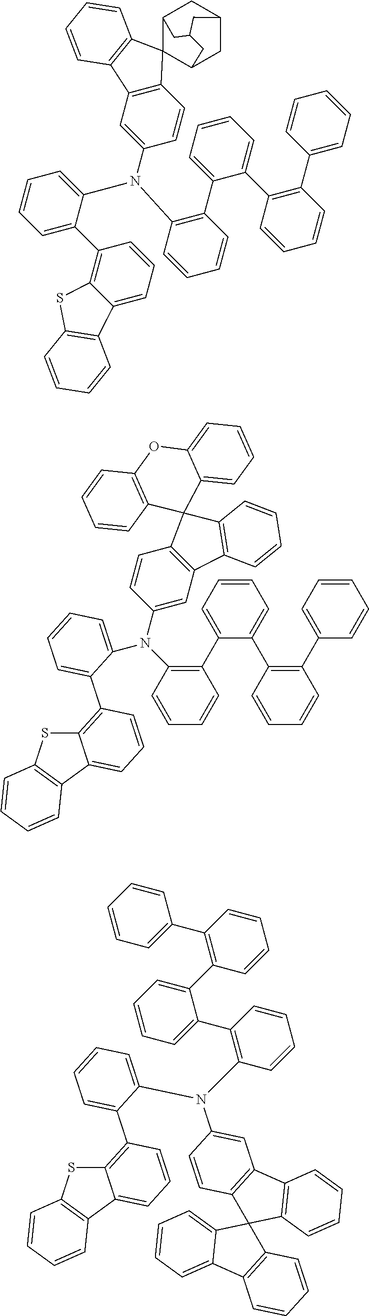 [SMILES: c1ccc(-c2ccccc2-c2ccccc2-c2ccccc2N(c2ccc3c(c2)-c2ccccc2C32C3CC4CC(C3)CC2C4)c2ccccc2-c2cccc3c2sc2ccccc23)cc1 ; c1ccc(-c2ccccc2-c2ccccc2-c2ccccc2N(c2ccc3c(c2)-c2ccccc2C32c3ccccc3Oc3ccccc32)c2ccccc2-c2cccc3c2sc2ccccc23)cc1 ; c1ccc(-c2ccccc2-c2ccccc2-c2ccccc2N(c2ccc3c(c2)-c2ccccc2C32c3ccccc3-c3ccccc32)c2ccccc2-c2cccc3c2sc2ccccc23)cc1]

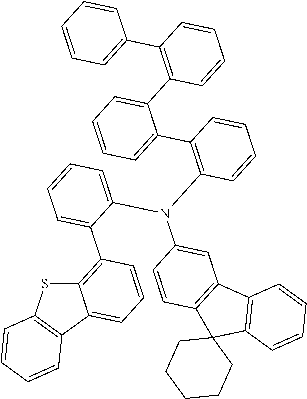 [SMILES: c1ccc(-c2ccccc2-c2ccccc2-c2ccccc2N(c2ccc3c(c2)-c2ccccc2C32CCCCC2)c2ccccc2-c2cccc3c2sc2ccccc23)cc1]

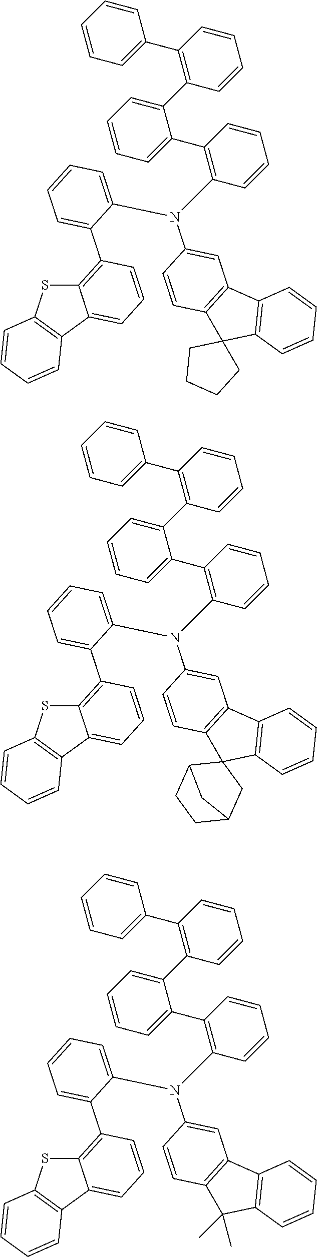 [SMILES: c1ccc(-c2ccccc2-c2ccccc2-c2ccccc2N(c2ccc3c(c2)-c2ccccc2C32CCCC2)c2ccccc2-c2cccc3c2sc2ccccc23)cc1 ; c1ccc(-c2ccccc2-c2ccccc2-c2ccccc2N(c2ccc3c(c2)-c2ccccc2C32CC3CCC2C3)c2ccccc2-c2cccc3c2sc2ccccc23)cc1 ; CC1(C)c2ccccc2-c2cc(N(c3ccccc3-c3ccccc3-c3ccccc3-c3ccccc3)c3ccccc3-c3cccc4c3sc3ccccc34)ccc21]

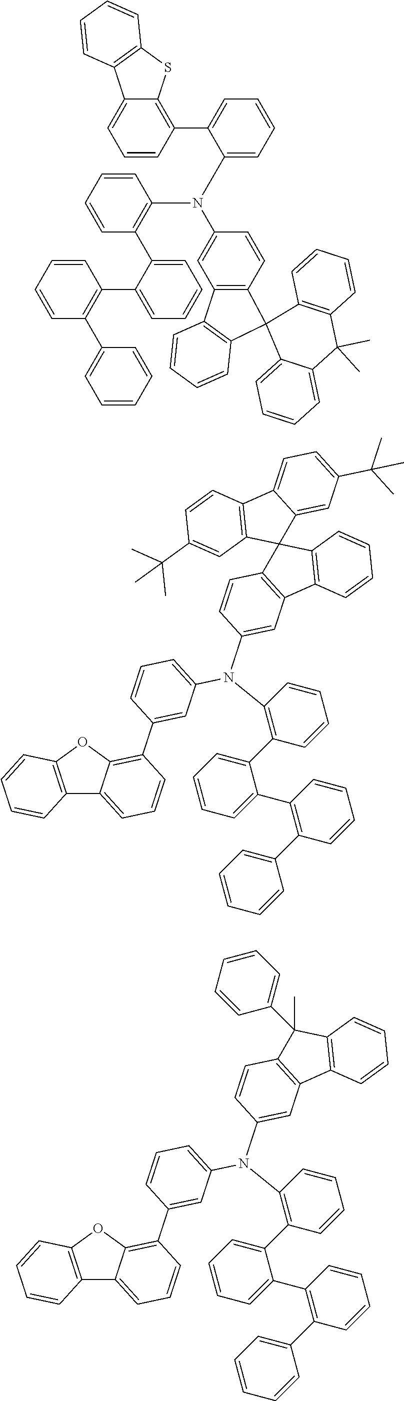

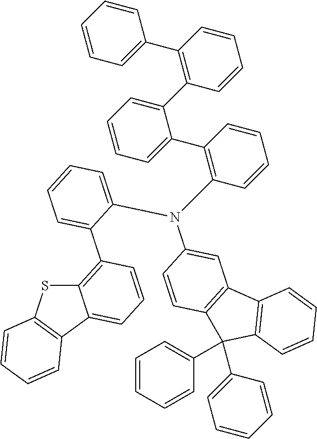 [SMILES: c1ccc(-c2ccccc2-c2ccccc2-c2ccccc2N(c2ccc3c(c2)-c2ccccc2C3(c2ccccc2)c2ccccc2)c2ccccc2-c2cccc3c2sc2ccccc23)cc1]

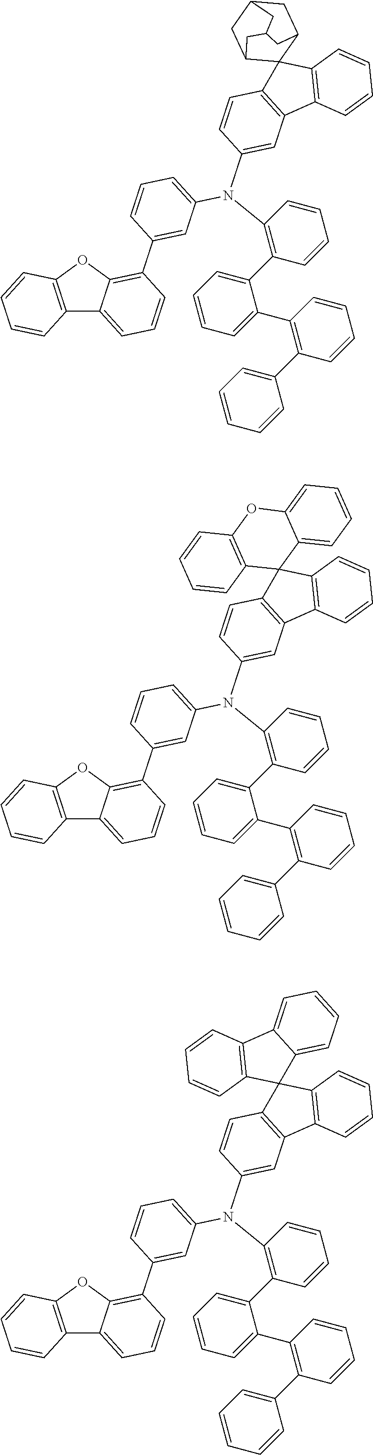 [SMILES: c1ccc(-c2ccccc2-c2ccccc2-c2ccccc2N(c2cccc(-c3cccc4c3oc3ccccc34)c2)c2ccc3c(c2)-c2ccccc2C32C3CC4CC(C3)CC2C4)cc1 ; c1ccc(-c2ccccc2-c2ccccc2-c2ccccc2N(c2cccc(-c3cccc4c3oc3ccccc34)c2)c2ccc3c(c2)-c2ccccc2C32c3ccccc3Oc3ccccc32)cc1 ; c1ccc(-c2ccccc2-c2ccccc2-c2ccccc2N(c2cccc(-c3cccc4c3oc3ccccc34)c2)c2ccc3c(c2)-c2ccccc2C32c3ccccc3-c3ccccc32)cc1]

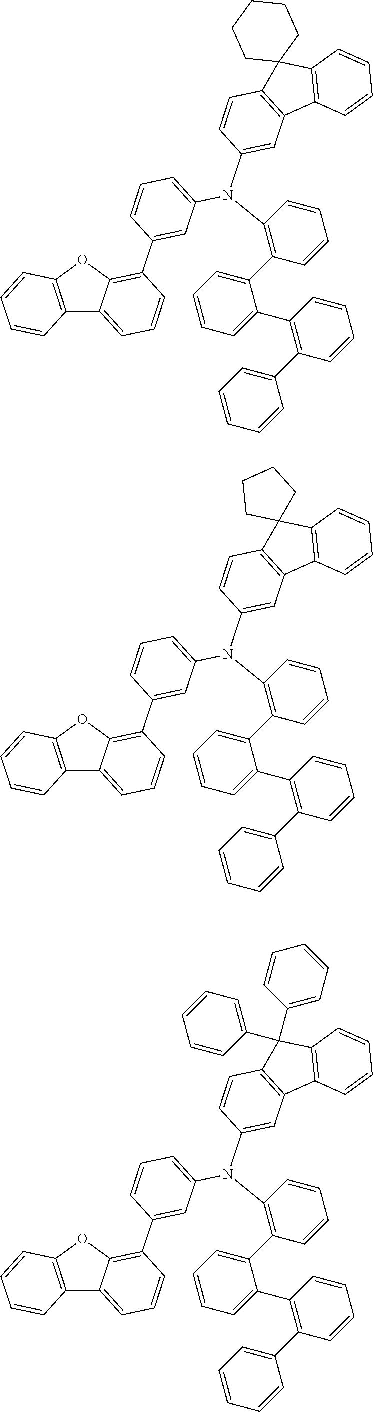

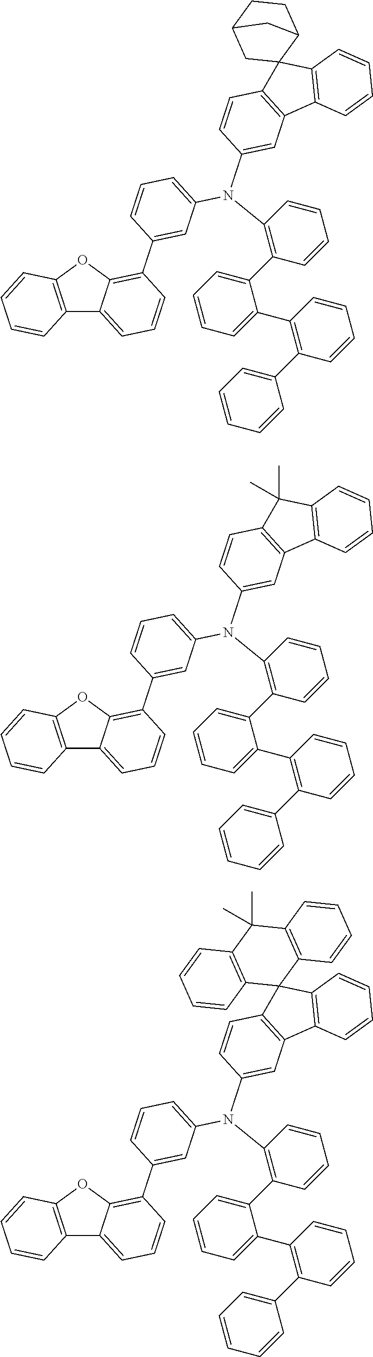

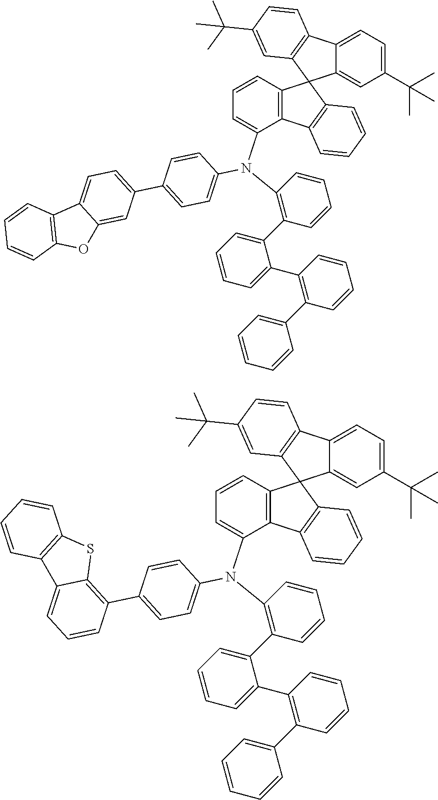

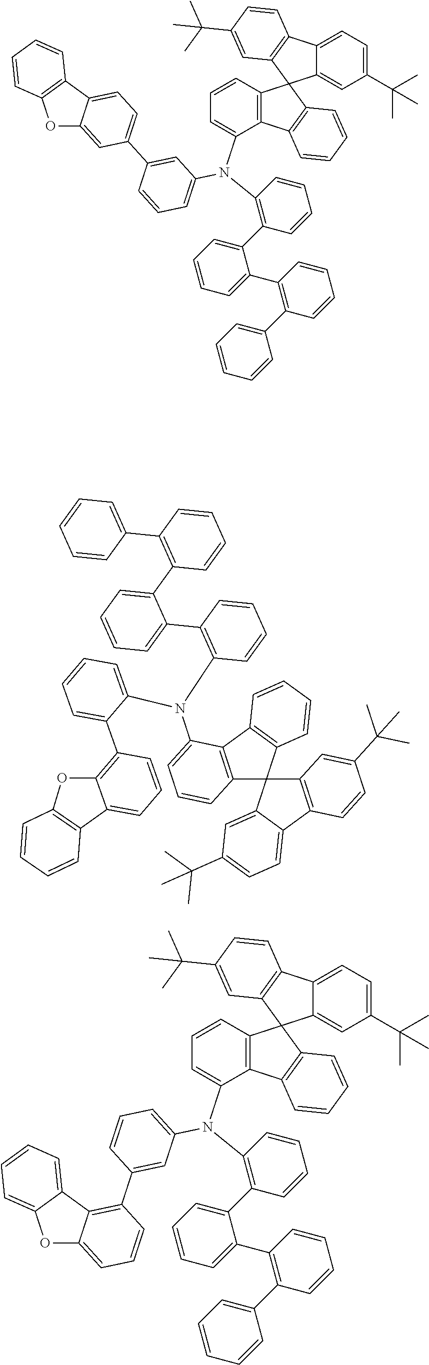

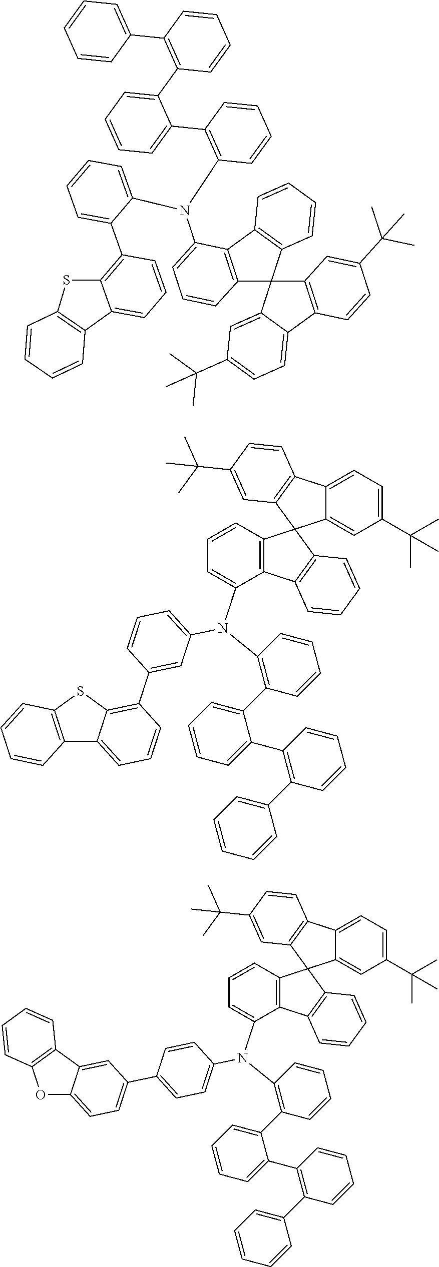

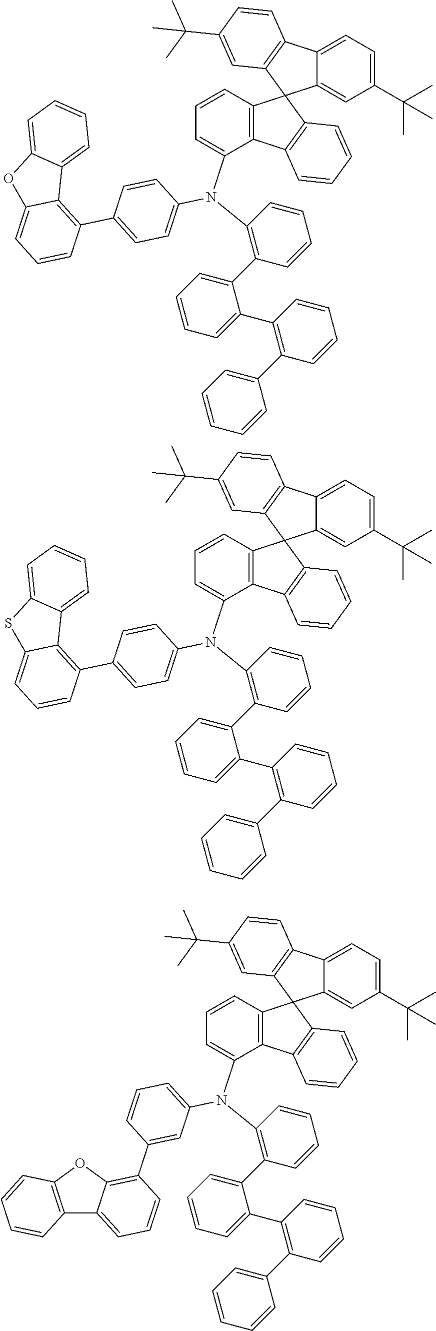

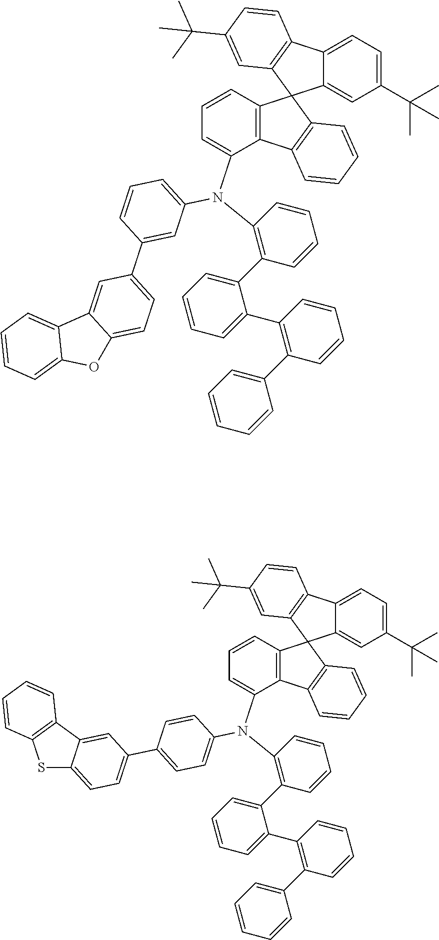 [SMILES: CC(C)(C)c1ccc2c(c1)C1(c3cc(C(C)(C)C)ccc3-2)c2ccccc2-c2c(N(c3cccc(-c4ccc5oc6ccccc6c5c4)c3)c3ccccc3-c3ccccc3-c3ccccc3-c3ccccc3)cccc21 ; CC(C)(C)c1ccc2c(c1)C1(c3cc(C(C)(C)C)ccc3-2)c2ccccc2-c2c(N(c3ccc(-c4ccc5sc6ccccc6c5c4)cc3)c3ccccc3-c3ccccc3-c3ccccc3-c3ccccc3)cccc21]

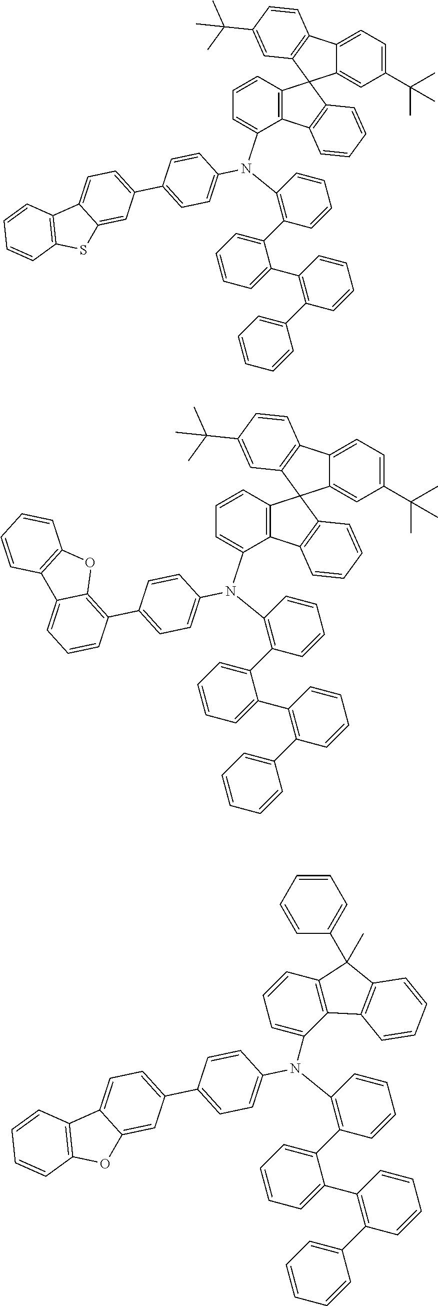 [SMILES: CC(C)(C)c1ccc2c(c1)C1(c3cc(C(C)(C)C)ccc3-2)c2ccccc2-c2c(N(c3ccc(-c4ccc5c(c4)sc4ccccc45)cc3)c3ccccc3-c3ccccc3-c3ccccc3-c3ccccc3)cccc21 ; CC(C)(C)c1ccc2c(c1)C1(c3cc(C(C)(C)C)ccc3-2)c2ccccc2-c2c(N(c3ccc(-c4cccc5c4oc4ccccc45)cc3)c3ccccc3-c3ccccc3-c3ccccc3-c3ccccc3)cccc21 ; CC1(c2ccccc2)c2ccccc2-c2c(N(c3ccc(-c4ccc5c(c4)oc4ccccc45)cc3)c3ccccc3-c3ccccc3-c3ccccc3-c3ccccc3)cccc21]

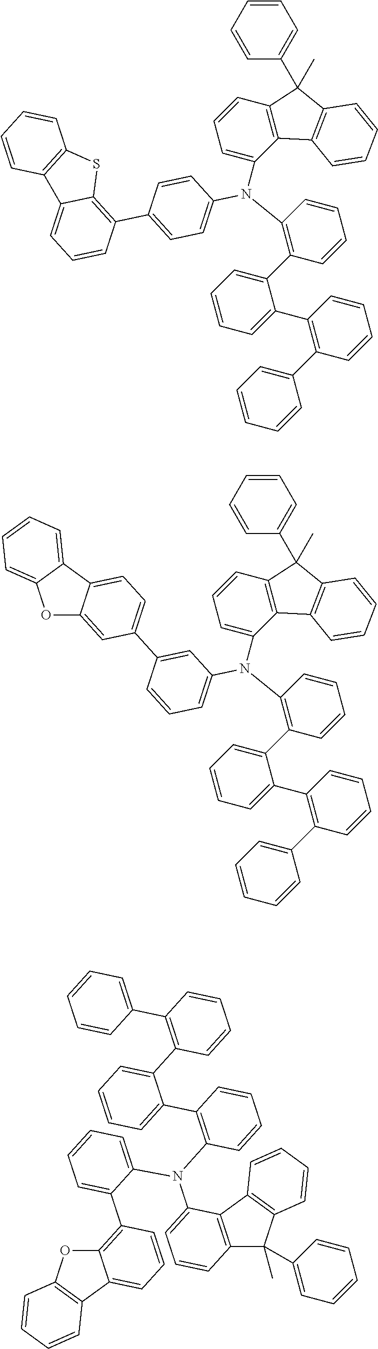

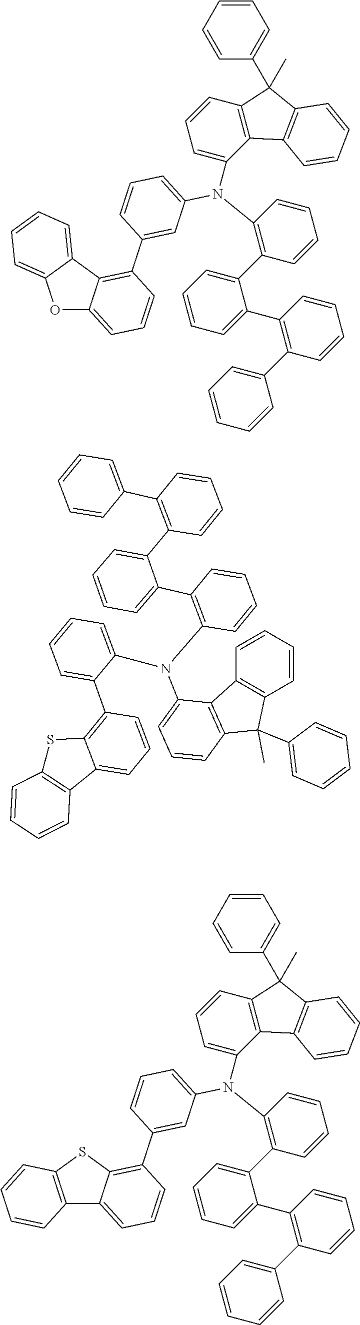 [SMILES: CC1(c2ccccc2)c2ccccc2-c2c(N(c3cccc(-c4cccc5oc6ccccc6c45)c3)c3ccccc3-c3ccccc3-c3ccccc3-c3ccccc3)cccc21 ; CC1(c2ccccc2)c2ccccc2-c2c(N(c3ccccc3-c3ccccc3-c3ccccc3-c3ccccc3)c3ccccc3-c3cccc4c3sc3ccccc34)cccc21 ; CC1(c2ccccc2)c2ccccc2-c2c(N(c3cccc(-c4cccc5c4sc4ccccc45)c3)c3ccccc3-c3ccccc3-c3ccccc3-c3ccccc3)cccc21]

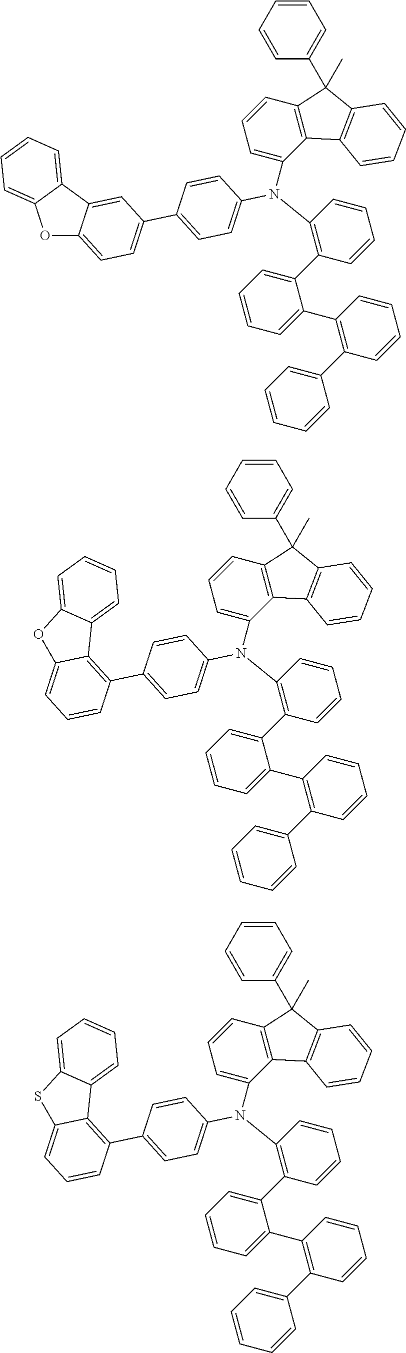

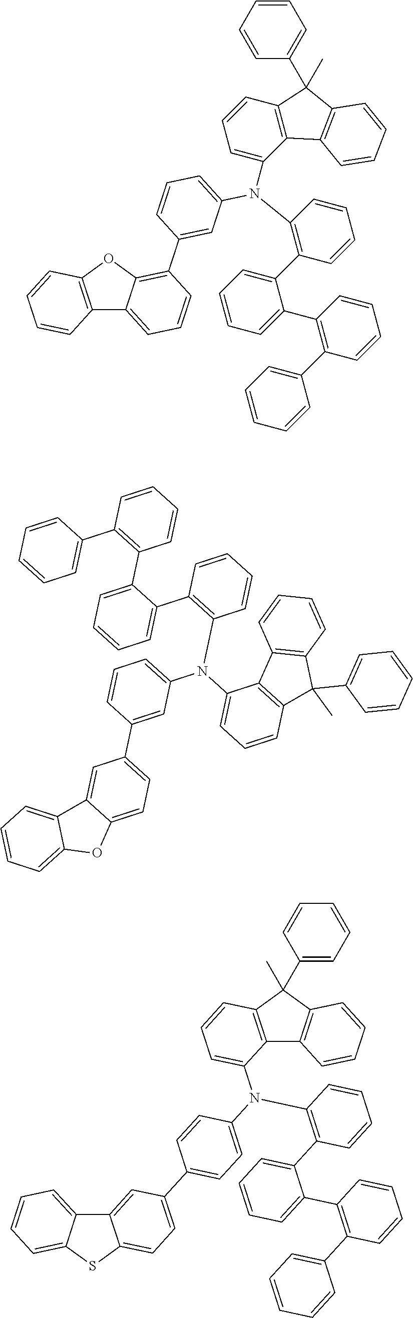 [SMILES: CC1(c2ccccc2)c2ccccc2-c2c(N(c3cccc(-c4cccc5c4oc4ccccc45)c3)c3ccccc3-c3ccccc3-c3ccccc3-c3ccccc3)cccc21 ; CC1(c2ccccc2)c2ccccc2-c2c(N(c3cccc(-c4ccc5oc6ccccc6c5c4)c3)c3ccccc3-c3ccccc3-c3ccccc3-c3ccccc3)cccc21 ; CC1(c2ccccc2)c2ccccc2-c2c(N(c3ccc(-c4ccc5sc6ccccc6c5c4)cc3)c3ccccc3-c3ccccc3-c3ccccc3-c3ccccc3)cccc21]

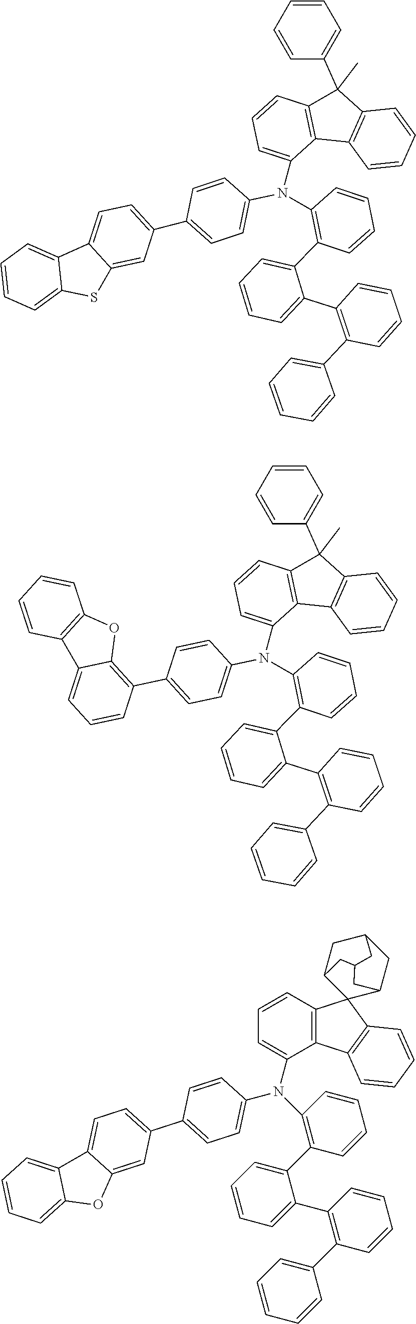

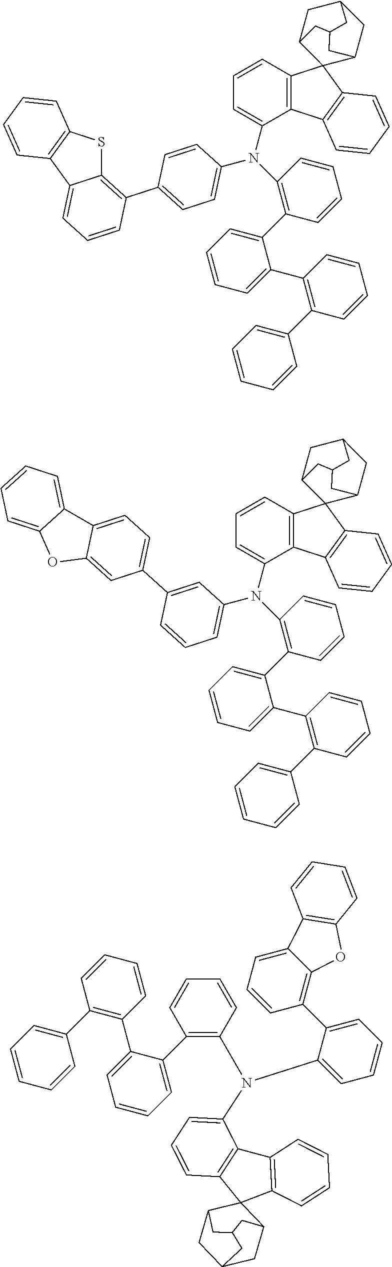 [SMILES: c1ccc(-c2ccccc2-c2ccccc2-c2ccccc2N(c2ccc(-c3cccc4c3sc3ccccc34)cc2)c2cccc3c2-c2ccccc2C32C3CC4CC(C3)CC2C4)cc1 ; c1ccc(-c2ccccc2-c2ccccc2-c2ccccc2N(c2cccc(-c3ccc4c(c3)oc3ccccc34)c2)c2cccc3c2-c2ccccc2C32C3CC4CC(C3)CC2C4)cc1 ; c1ccc(-c2ccccc2-c2ccccc2-c2ccccc2N(c2ccccc2-c2cccc3c2oc2ccccc23)c2cccc3c2-c2ccccc2C32C3CC4CC(C3)CC2C4)cc1]

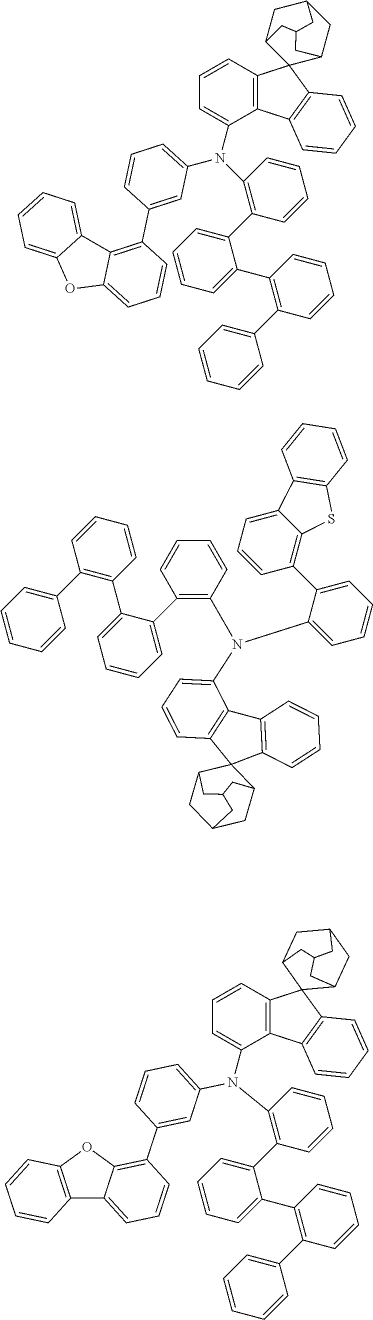 [SMILES: c1ccc(-c2ccccc2-c2ccccc2-c2ccccc2N(c2cccc(-c3cccc4oc5ccccc5c34)c2)c2cccc3c2-c2ccccc2C32C3CC4CC(C3)CC2C4)cc1 ; c1ccc(-c2ccccc2-c2ccccc2-c2ccccc2N(c2ccccc2-c2cccc3c2sc2ccccc23)c2cccc3c2-c2ccccc2C32C3CC4CC(C3)CC2C4)cc1 ; c1ccc(-c2ccccc2-c2ccccc2-c2ccccc2N(c2cccc(-c3cccc4c3oc3ccccc34)c2)c2cccc3c2-c2ccccc2C32C3CC4CC(C3)CC2C4)cc1]

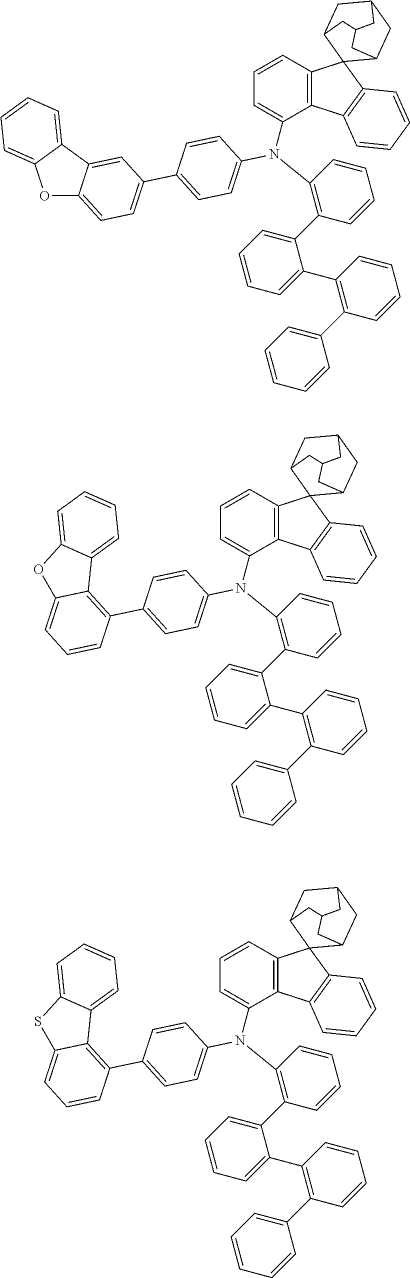 [SMILES: c1ccc(-c2ccccc2-c2ccccc2-c2ccccc2N(c2ccc(-c3ccc4oc5ccccc5c4c3)cc2)c2cccc3c2-c2ccccc2C32C3CC4CC(C3)CC2C4)cc1 ; c1ccc(-c2ccccc2-c2ccccc2-c2ccccc2N(c2ccc(-c3cccc4oc5ccccc5c34)cc2)c2cccc3c2-c2ccccc2C32C3CC4CC(C3)CC2C4)cc1 ; c1ccc(-c2ccccc2-c2ccccc2-c2ccccc2N(c2ccc(-c3cccc4sc5ccccc5c34)cc2)c2cccc3c2-c2ccccc2C32C3CC4CC(C3)CC2C4)cc1]

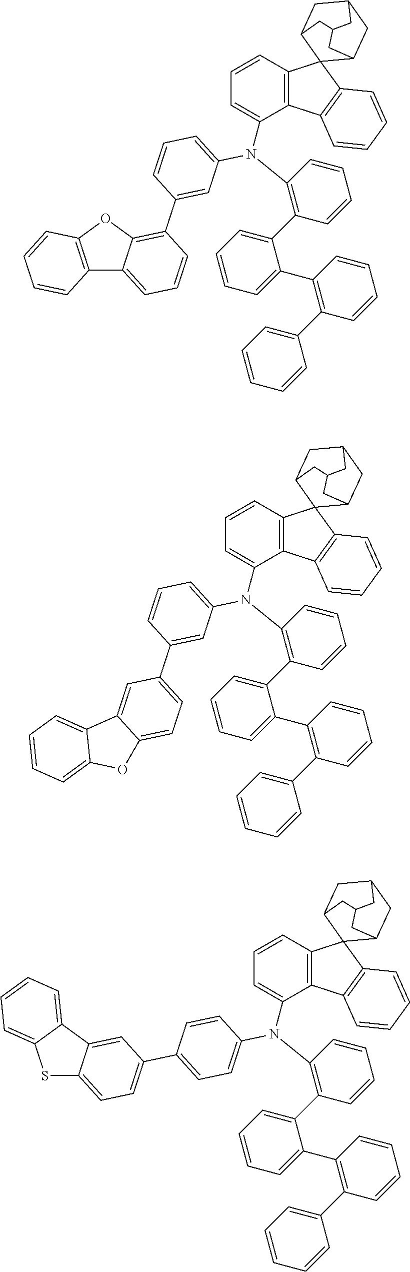 [SMILES: c1ccc(-c2ccccc2-c2ccccc2-c2ccccc2N(c2cccc(-c3cccc4c3oc3ccccc34)c2)c2cccc3c2-c2ccccc2C32C3CC4CC(C3)CC2C4)cc1 ; c1ccc(-c2ccccc2-c2ccccc2-c2ccccc2N(c2cccc(-c3ccc4oc5ccccc5c4c3)c2)c2cccc3c2-c2ccccc2C32C3CC4CC(C3)CC2C4)cc1 ; c1ccc(-c2ccccc2-c2ccccc2-c2ccccc2N(c2ccc(-c3ccc4sc5ccccc5c4c3)cc2)c2cccc3c2-c2ccccc2C32C3CC4CC(C3)CC2C4)cc1]

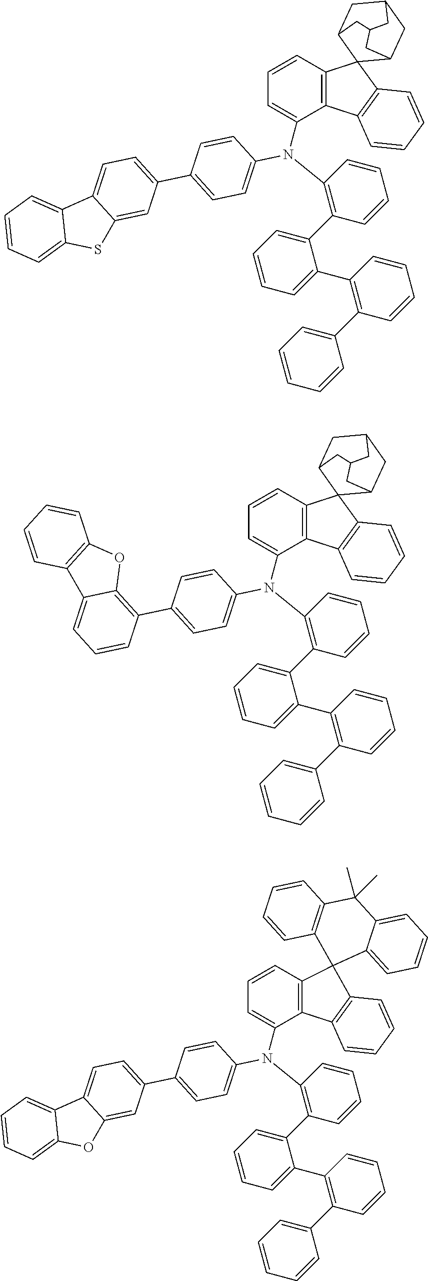 [SMILES: c1ccc(-c2ccccc2-c2ccccc2-c2ccccc2N(c2ccc(-c3ccc4c(c3)sc3ccccc34)cc2)c2cccc3c2-c2ccccc2C32C3CC4CC(C3)CC2C4)cc1 ; c1ccc(-c2ccccc2-c2ccccc2-c2ccccc2N(c2ccc(-c3cccc4c3oc3ccccc34)cc2)c2cccc3c2-c2ccccc2C32C3CC4CC(C3)CC2C4)cc1 ; CC1(C)c2ccccc2C2(c3ccccc3-c3c(N(c4ccc(-c5ccc6c(c5)oc5ccccc56)cc4)c4ccccc4-c4ccccc4-c4ccccc4-c4ccccc4)cccc32)c2ccccc21]

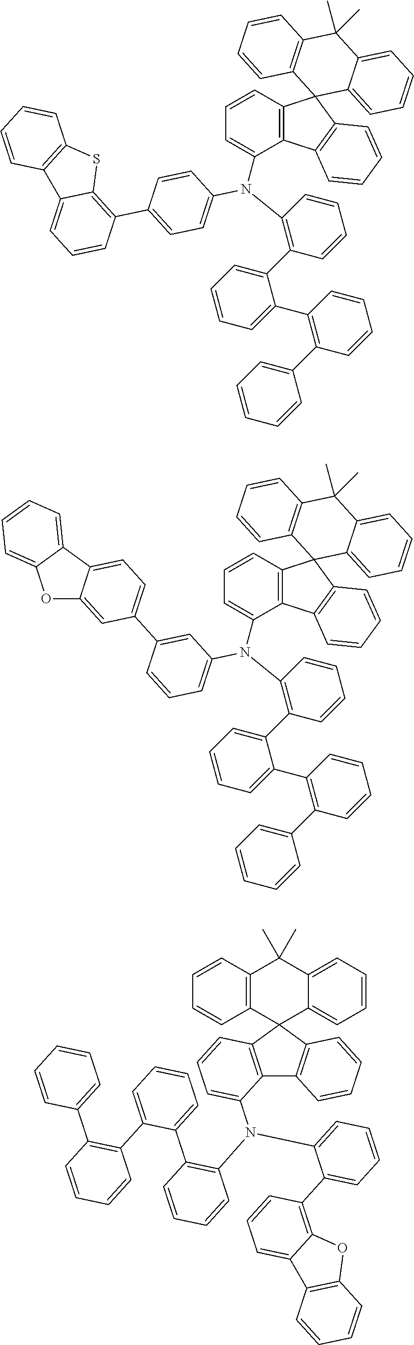

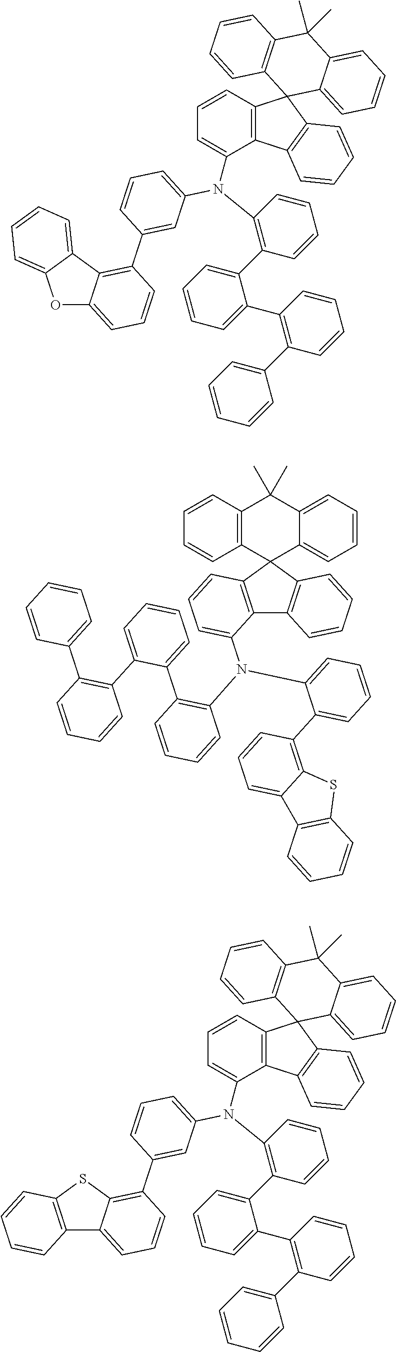

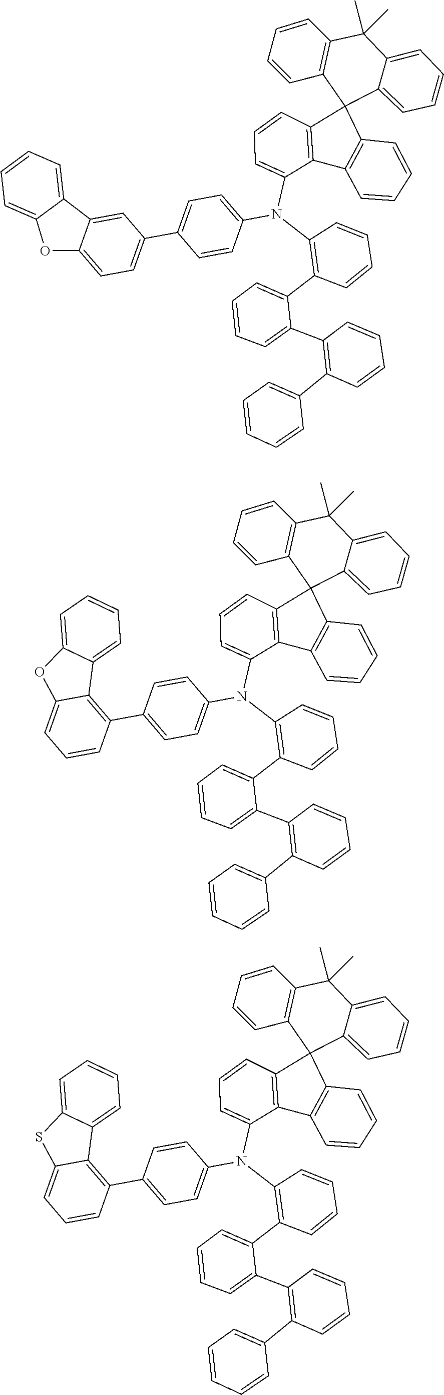

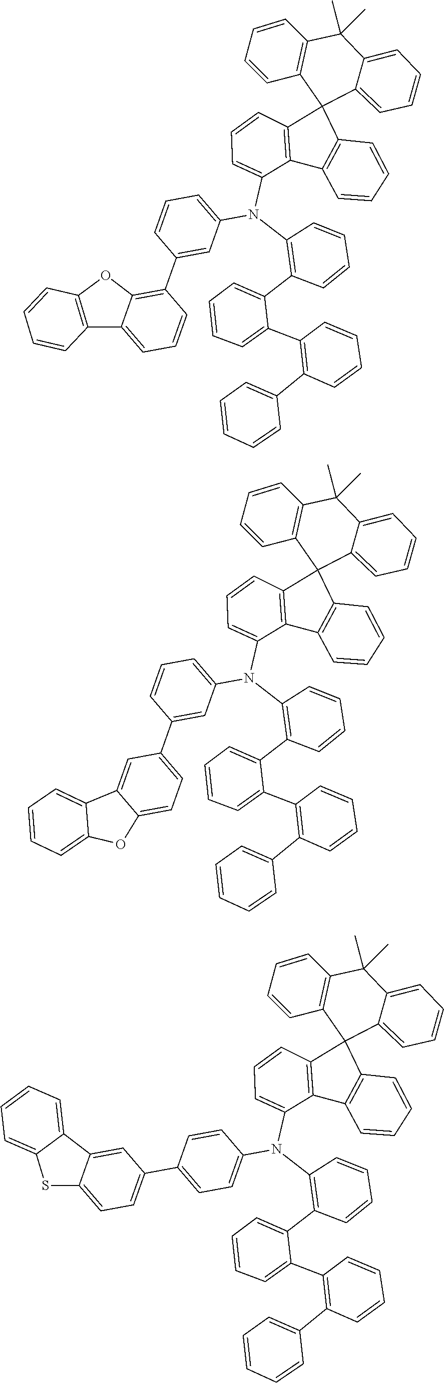

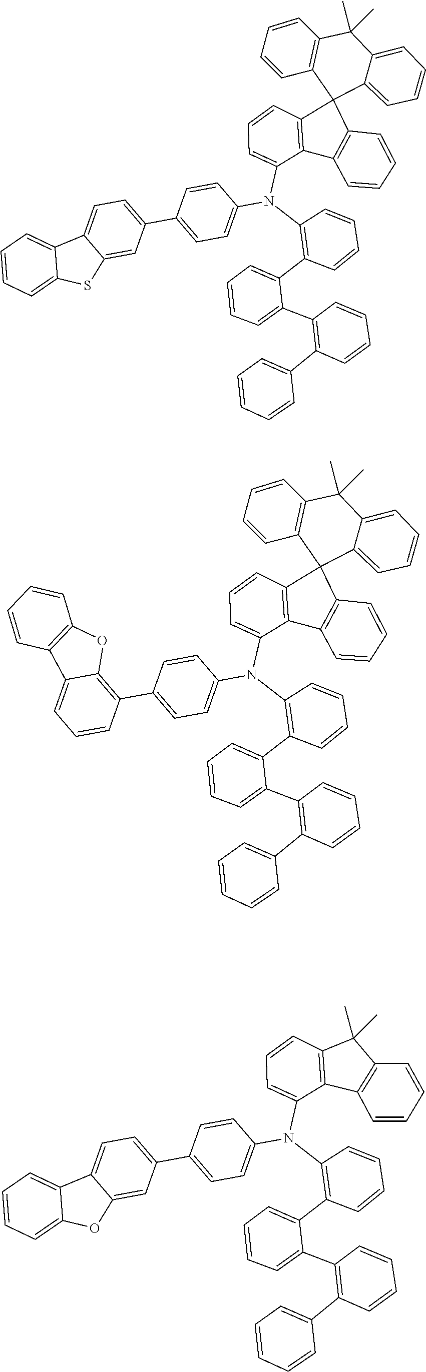 [SMILES: CC1(C)c2ccccc2C2(c3ccccc3-c3c(N(c4ccc(-c5ccc6c(c5)sc5ccccc56)cc4)c4ccccc4-c4ccccc4-c4ccccc4-c4ccccc4)cccc32)c2ccccc21 ; CC1(C)c2ccccc2C2(c3ccccc3-c3c(N(c4ccc(-c5cccc6c5oc5ccccc56)cc4)c4ccccc4-c4ccccc4-c4ccccc4-c4ccccc4)cccc32)c2ccccc21 ; CC1(C)c2ccccc2-c2c(N(c3ccc(-c4ccc5c(c4)oc4ccccc45)cc3)c3ccccc3-c3ccccc3-c3ccccc3-c3ccccc3)cccc21]

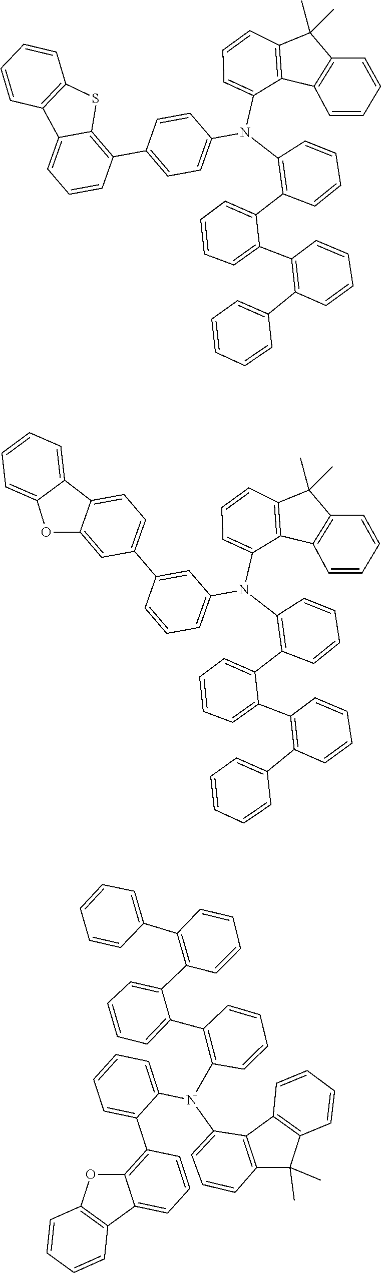 [SMILES: CC1(C)c2ccccc2-c2c(N(c3ccc(-c4cccc5c4sc4ccccc45)cc3)c3ccccc3-c3ccccc3-c3ccccc3-c3ccccc3)cccc21 ; CC1(C)c2ccccc2-c2c(N(c3cccc(-c4ccc5c(c4)oc4ccccc45)c3)c3ccccc3-c3ccccc3-c3ccccc3-c3ccccc3)cccc21 ; CC1(C)c2ccccc2-c2c(N(c3ccccc3-c3ccccc3-c3ccccc3-c3ccccc3)c3ccccc3-c3cccc4c3oc3ccccc34)cccc21]

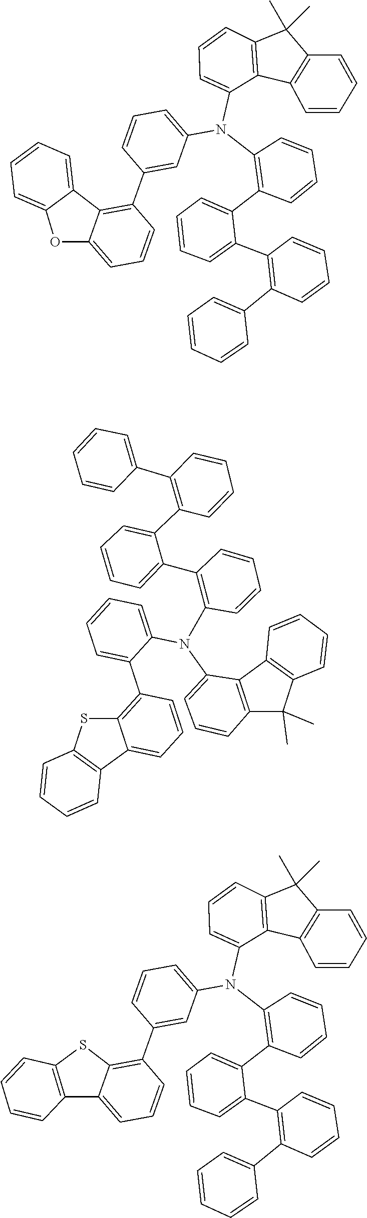 [SMILES: CC1(C)c2ccccc2-c2c(N(c3cccc(-c4cccc5oc6ccccc6c45)c3)c3ccccc3-c3ccccc3-c3ccccc3-c3ccccc3)cccc21 ; CC1(C)c2ccccc2-c2c(N(c3ccccc3-c3ccccc3-c3ccccc3-c3ccccc3)c3ccccc3-c3cccc4c3sc3ccccc34)cccc21 ; CC1(C)c2ccccc2-c2c(N(c3cccc(-c4cccc5c4sc4ccccc45)c3)c3ccccc3-c3ccccc3-c3ccccc3-c3ccccc3)cccc21]

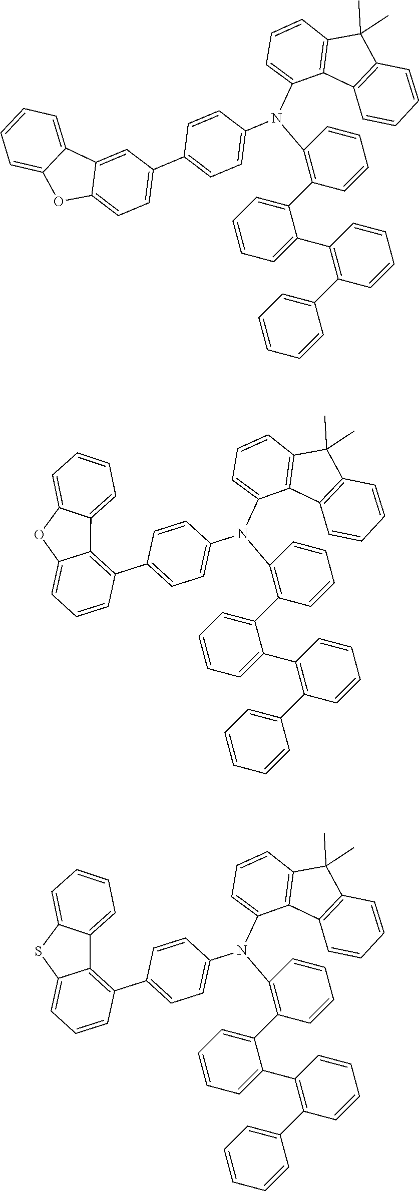 [SMILES: CC1(C)c2ccccc2-c2c(N(c3ccc(-c4ccc5oc6ccccc6c5c4)cc3)c3ccccc3-c3ccccc3-c3ccccc3-c3ccccc3)cccc21 ; CC1(C)c2ccccc2-c2c(N(c3ccc(-c4cccc5oc6ccccc6c45)cc3)c3ccccc3-c3ccccc3-c3ccccc3-c3ccccc3)cccc21 ; CC1(C)c2ccccc2-c2c(N(c3ccc(-c4cccc5sc6ccccc6c45)cc3)c3ccccc3-c3ccccc3-c3ccccc3-c3ccccc3)cccc21]

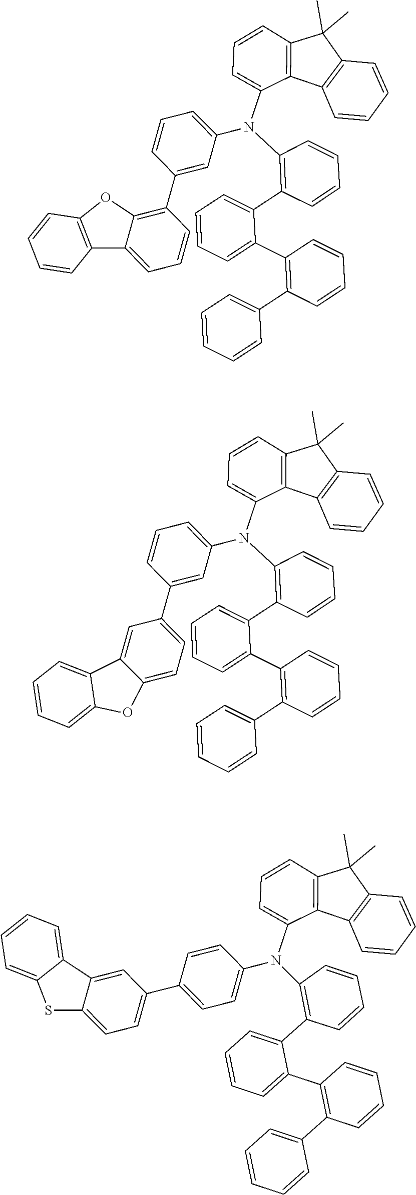 [SMILES: CC1(C)c2ccccc2-c2c(N(c3cccc(-c4cccc5c4oc4ccccc45)c3)c3ccccc3-c3ccccc3-c3ccccc3-c3ccccc3)cccc21 ; CC1(C)c2ccccc2-c2c(N(c3cccc(-c4ccc5oc6ccccc6c5c4)c3)c3ccccc3-c3ccccc3-c3ccccc3-c3ccccc3)cccc21 ; CC1(C)c2ccccc2-c2c(N(c3ccc(-c4ccc5sc6ccccc6c5c4)cc3)c3ccccc3-c3ccccc3-c3ccccc3-c3ccccc3)cccc21]

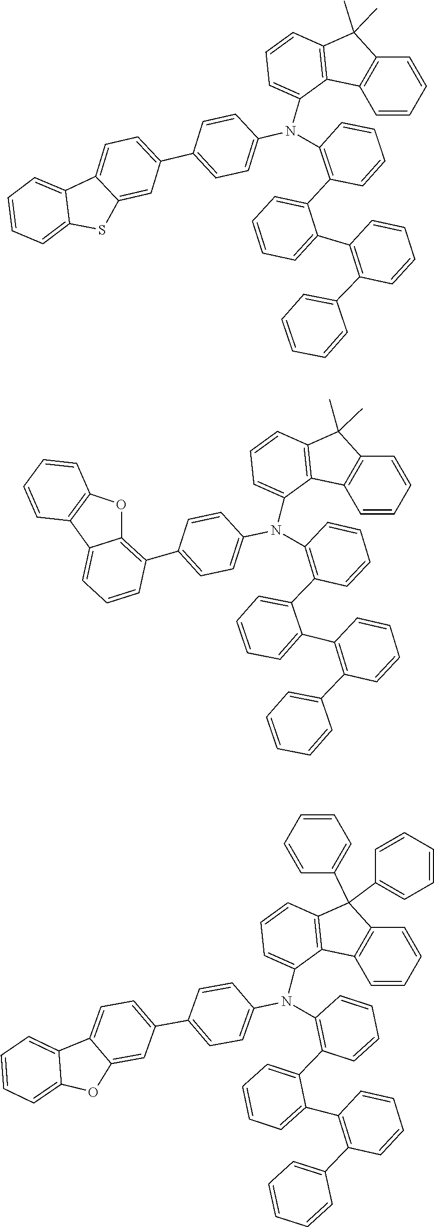

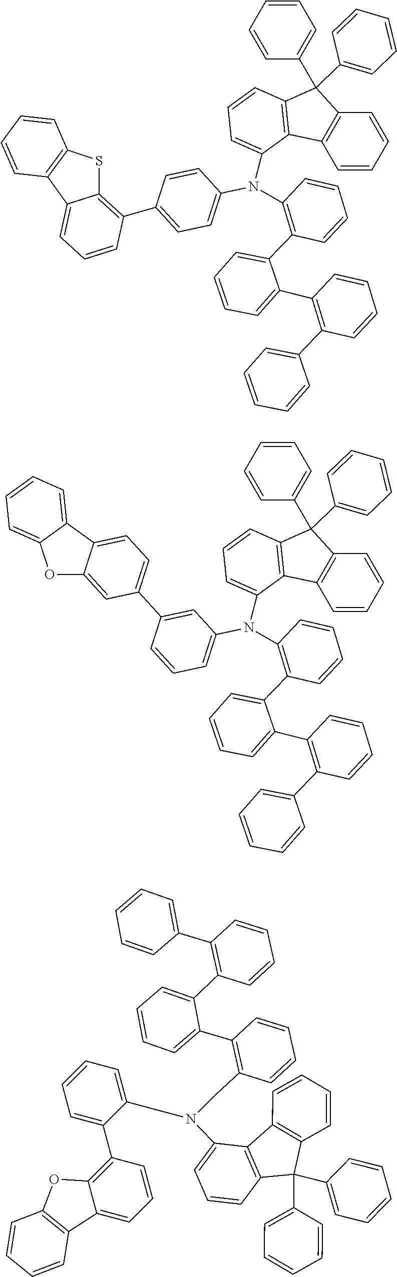

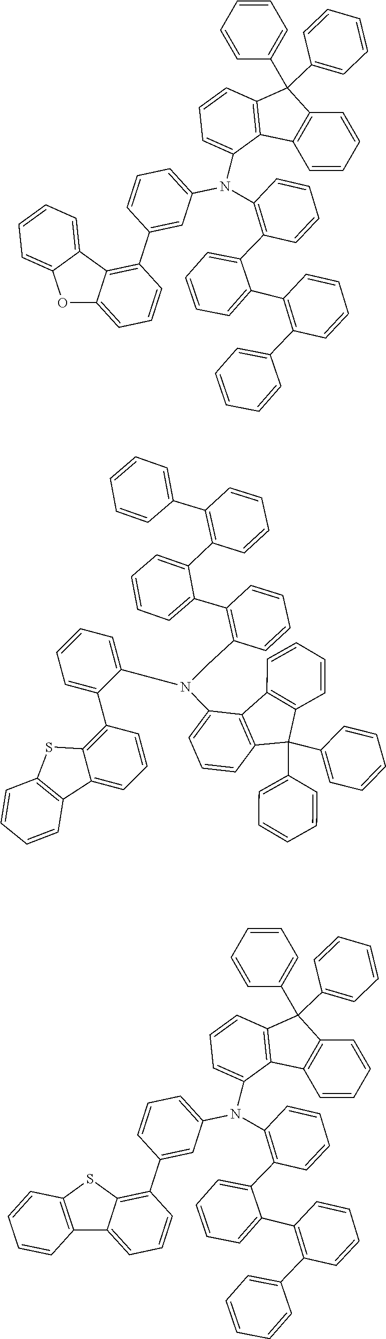 [SMILES: c1ccc(-c2ccccc2-c2ccccc2-c2ccccc2N(c2cccc(-c3cccc4oc5ccccc5c34)c2)c2cccc3c2-c2ccccc2C3(c2ccccc2)c2ccccc2)cc1 ; c1ccc(-c2ccccc2-c2ccccc2-c2ccccc2CN(Cc2ccccc2-c2cccc3c2sc2ccccc23)c2cccc3c2-c2ccccc2C3(c2ccccc2)c2ccccc2)cc1 ; c1ccc(-c2ccccc2-c2ccccc2-c2ccccc2N(c2cccc(-c3cccc4c3sc3ccccc34)c2)c2cccc3c2-c2ccccc2C3(c2ccccc2)c2ccccc2)cc1]

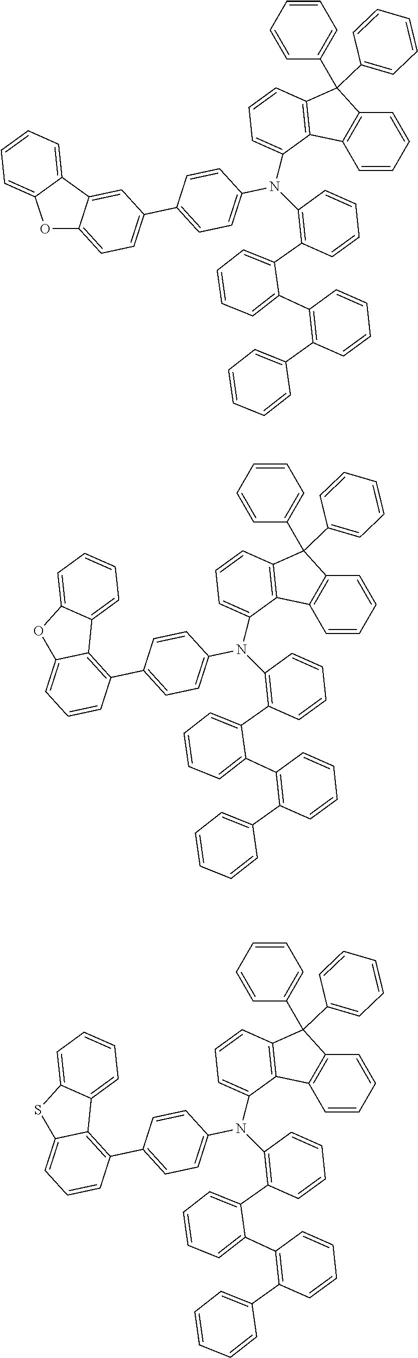 [SMILES: c1ccc(-c2ccccc2-c2ccccc2-c2ccccc2N(c2ccc(-c3ccc4oc5ccccc5c4c3)cc2)c2cccc3c2-c2ccccc2C3(c2ccccc2)c2ccccc2)cc1 ; c1ccc(-c2ccccc2-c2ccccc2-c2ccccc2N(c2ccc(-c3cccc4oc5ccccc5c34)cc2)c2cccc3c2-c2ccccc2C3(c2ccccc2)c2ccccc2)cc1 ; c1ccc(-c2ccccc2-c2ccccc2-c2ccccc2N(c2ccc(-c3cccc4sc5ccccc5c34)cc2)c2cccc3c2-c2ccccc2C3(c2ccccc2)c2ccccc2)cc1]

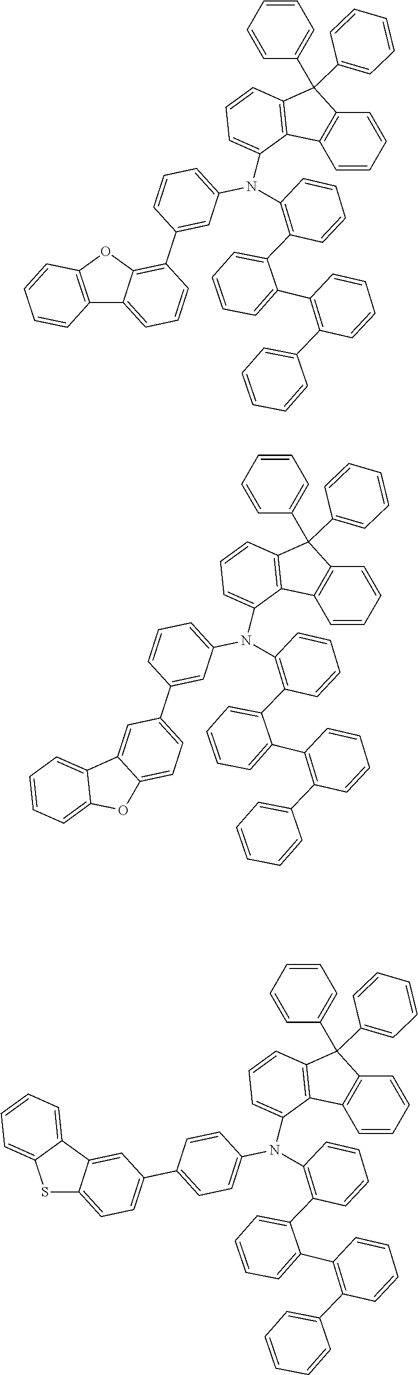

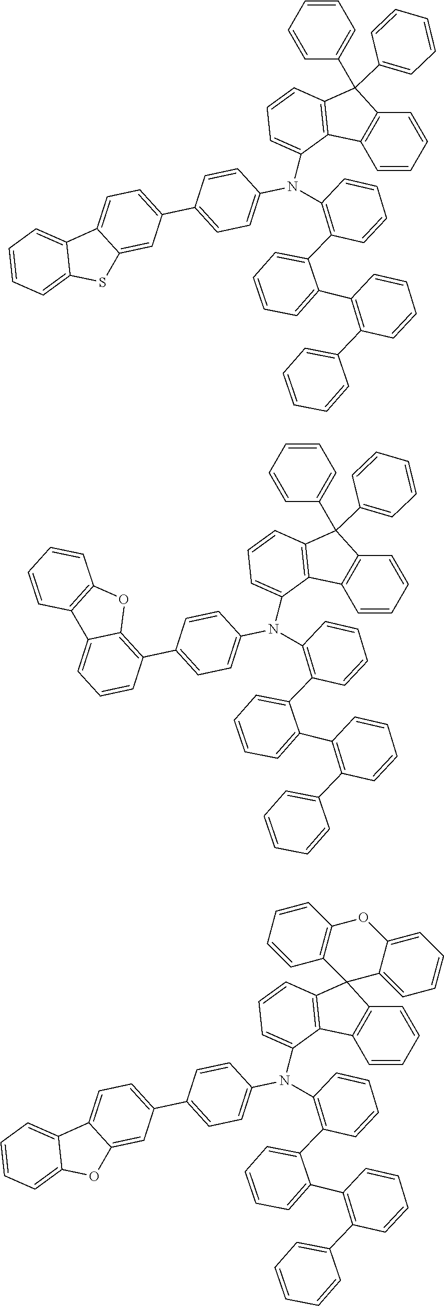

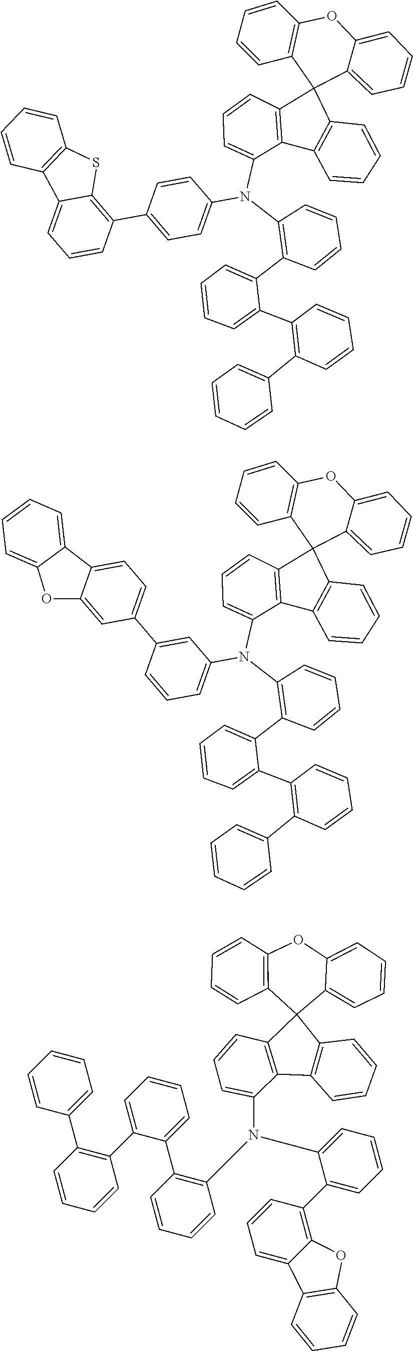

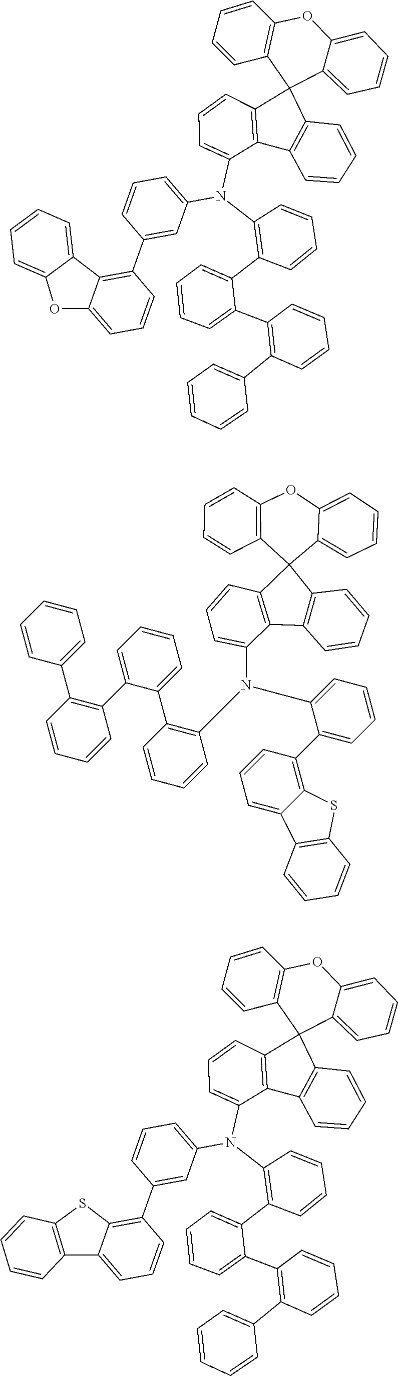 [SMILES: c1ccc(-c2ccccc2-c2ccccc2-c2ccccc2N(c2cccc(-c3cccc4oc5ccccc5c34)c2)c2cccc3c2-c2ccccc2C32c3ccccc3Oc3ccccc32)cc1 ; c1ccc(-c2ccccc2-c2ccccc2-c2ccccc2CN(Cc2ccccc2-c2cccc3c2sc2ccccc23)c2cccc3c2-c2ccccc2C32c3ccccc3Oc3ccccc32)cc1 ; c1ccc(-c2ccccc2-c2ccccc2-c2ccccc2N(c2cccc(-c3cccc4c3sc3ccccc34)c2)c2cccc3c2-c2ccccc2C32c3ccccc3Oc3ccccc32)cc1]

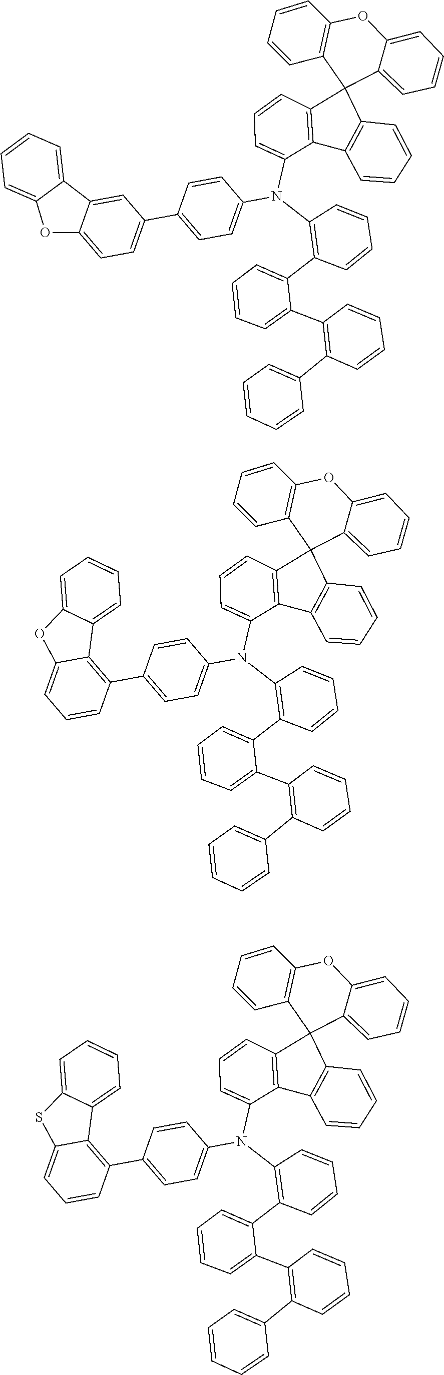

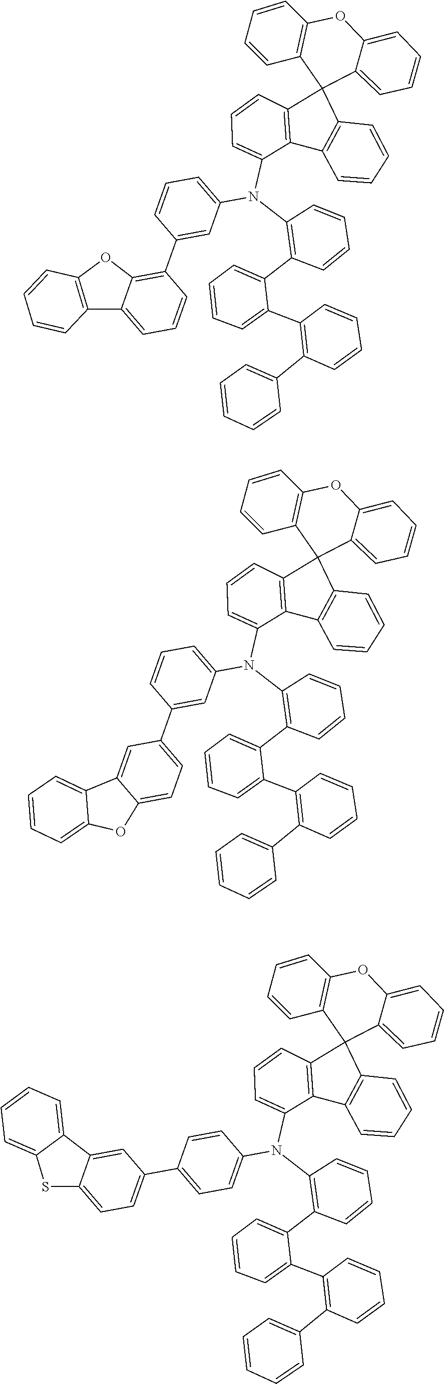 [SMILES: c1ccc(-c2ccccc2-c2ccccc2-c2ccccc2N(c2cccc(-c3cccc4c3oc3ccccc34)c2)c2cccc3c2-c2ccccc2C32c3ccccc3Oc3ccccc32)cc1 ; c1ccc(-c2ccccc2-c2ccccc2-c2ccccc2N(c2cccc(-c3ccc4oc5ccccc5c4c3)c2)c2cccc3c2-c2ccccc2C32c3ccccc3Oc3ccccc32)cc1 ; c1ccc(-c2ccccc2-c2ccccc2-c2ccccc2N(c2ccc(-c3ccc4sc5ccccc5c4c3)cc2)c2cccc3c2-c2ccccc2C32c3ccccc3Oc3ccccc32)cc1]

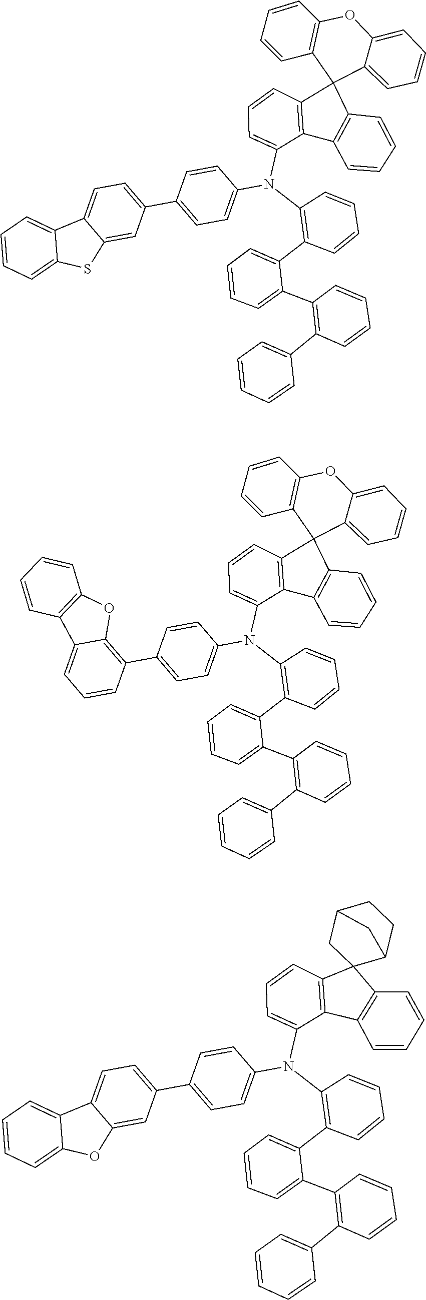 [SMILES: c1ccc(-c2ccccc2-c2ccccc2-c2ccccc2N(c2ccc(-c3ccc4c(c3)sc3ccccc34)cc2)c2cccc3c2-c2ccccc2C32c3ccccc3Oc3ccccc32)cc1 ; c1ccc(-c2ccccc2-c2ccccc2-c2ccccc2N(c2ccc(-c3cccc4c3oc3ccccc34)cc2)c2cccc3c2-c2ccccc2C32c3ccccc3Oc3ccccc32)cc1 ; c1ccc(-c2ccccc2-c2ccccc2-c2ccccc2N(c2ccc(-c3ccc4c(c3)oc3ccccc34)cc2)c2cccc3c2-c2ccccc2C32CC3CCC2C3)cc1]

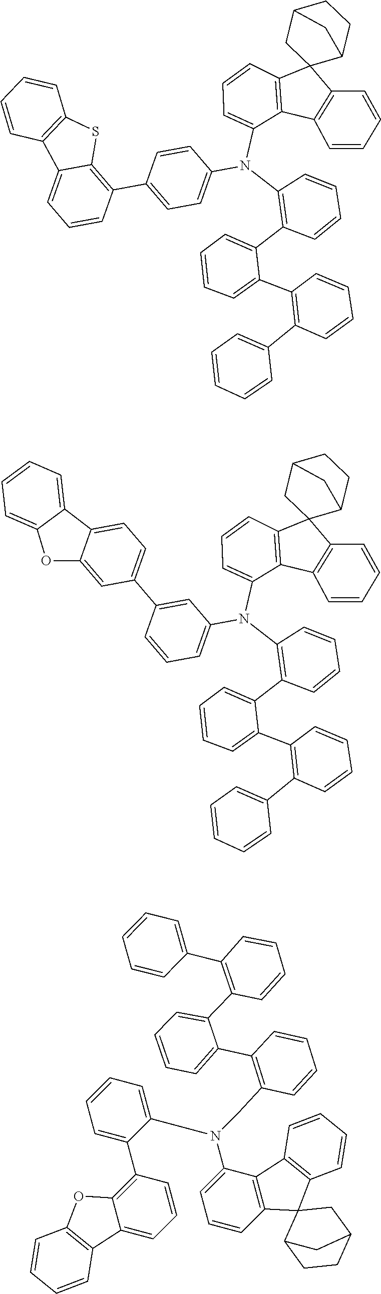 [SMILES: c1ccc(-c2ccccc2-c2ccccc2-c2ccccc2N(c2ccc(-c3cccc4c3sc3ccccc34)cc2)c2cccc3c2-c2ccccc2C32CC3CCC2C3)cc1 ; c1ccc(-c2ccccc2-c2ccccc2-c2ccccc2N(c2cccc(-c3ccc4c(c3)oc3ccccc34)c2)c2cccc3c2-c2ccccc2C32CC3CCC2C3)cc1 ; c1ccc(-c2ccccc2-c2ccccc2-c2ccccc2CN(Cc2ccccc2-c2cccc3c2oc2ccccc23)c2cccc3c2-c2ccccc2C32CC3CCC2C3)cc1]

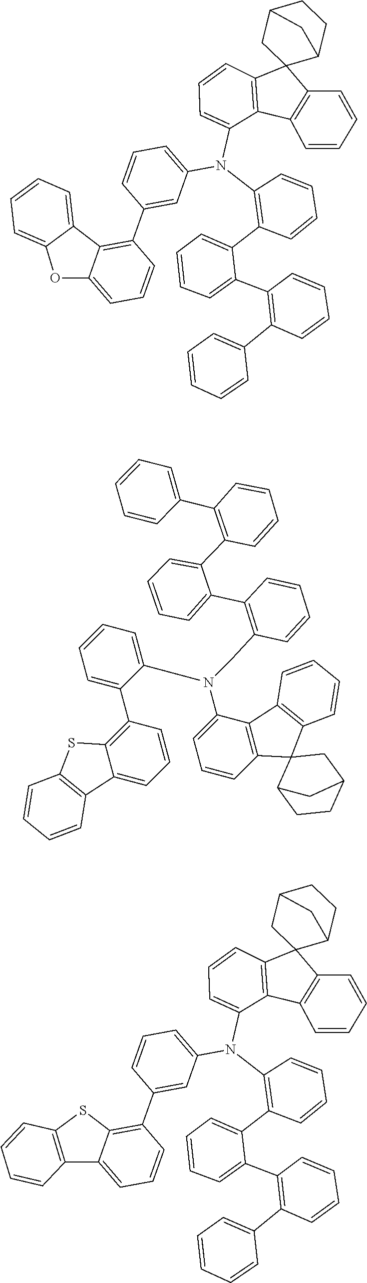 [SMILES: c1ccc(-c2ccccc2-c2ccccc2-c2ccccc2N(c2cccc(-c3cccc4oc5ccccc5c34)c2)c2cccc3c2-c2ccccc2C32CC3CCC2C3)cc1 ; c1ccc(-c2ccccc2-c2ccccc2-c2ccccc2CN(Cc2ccccc2-c2cccc3c2sc2ccccc23)c2cccc3c2-c2ccccc2C32CC3CCC2C3)cc1 ; c1ccc(-c2ccccc2-c2ccccc2-c2ccccc2N(c2cccc(-c3cccc4c3sc3ccccc34)c2)c2cccc3c2-c2ccccc2C32CC3CCC2C3)cc1]

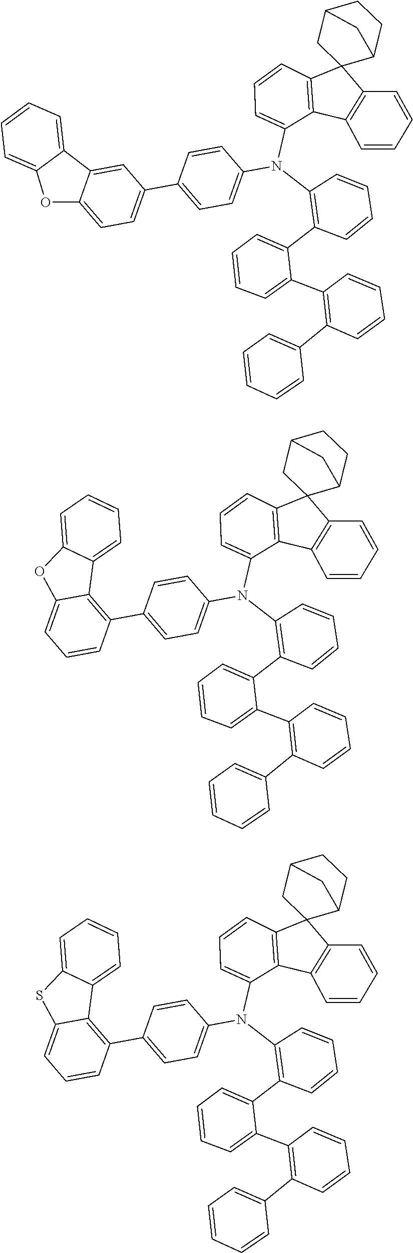

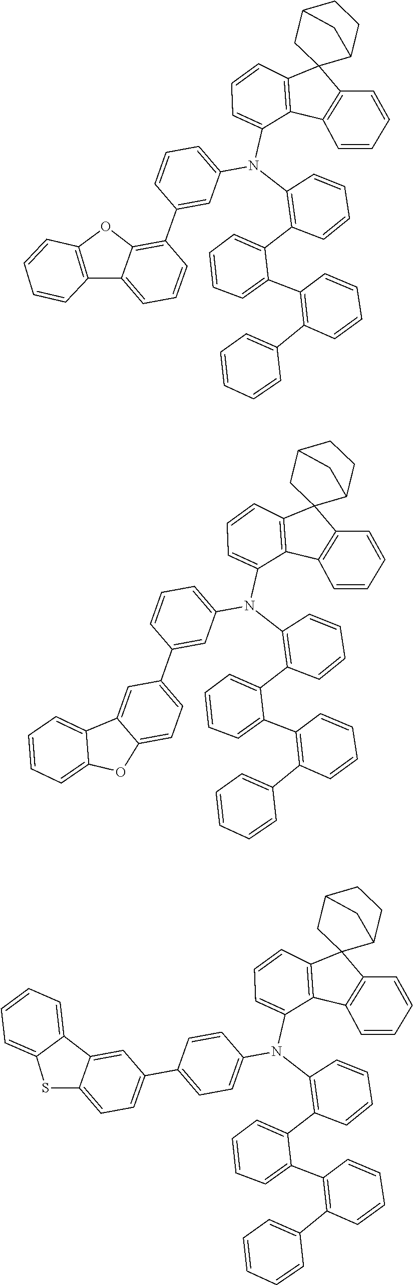 [SMILES: c1ccc(-c2ccccc2-c2ccccc2-c2ccccc2N(c2cccc(-c3cccc4c3oc3ccccc34)c2)c2cccc3c2-c2ccccc2C32CC3CCC2C3)cc1 ; c1ccc(-c2ccccc2-c2ccccc2-c2ccccc2N(c2cccc(-c3ccc4oc5ccccc5c4c3)c2)c2cccc3c2-c2ccccc2C32CC3CCC2C3)cc1 ; c1ccc(-c2ccccc2-c2ccccc2-c2ccccc2N(c2ccc(-c3ccc4sc5ccccc5c4c3)cc2)c2cccc3c2-c2ccccc2C32CC3CCC2C3)cc1]

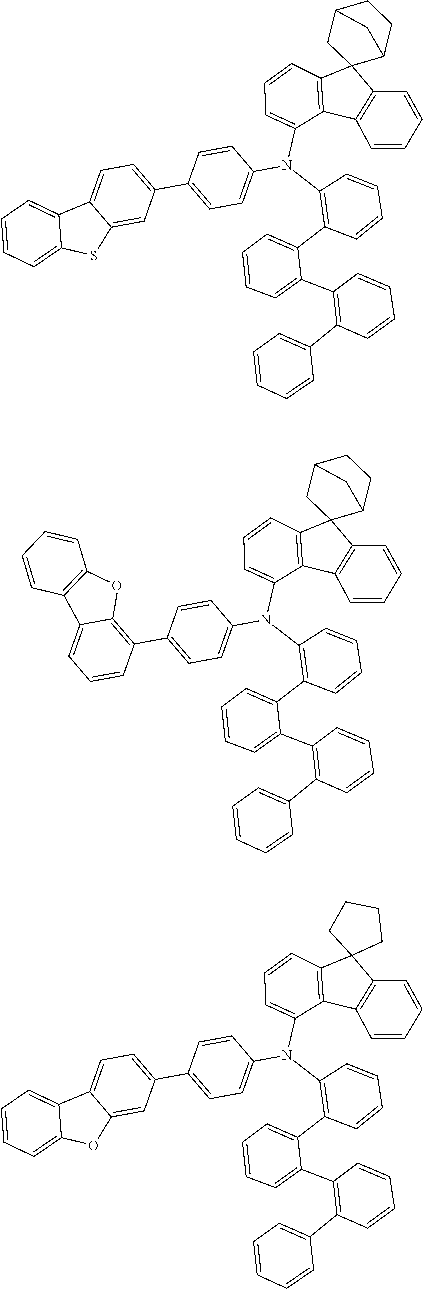 [SMILES: c1ccc(-c2ccccc2-c2ccccc2-c2ccccc2N(c2ccc(-c3ccc4c(c3)sc3ccccc34)cc2)c2cccc3c2-c2ccccc2C32CC3CCC2C3)cc1 ; c1ccc(-c2ccccc2-c2ccccc2-c2ccccc2N(c2ccc(-c3cccc4c3oc3ccccc34)cc2)c2cccc3c2-c2ccccc2C32CC3CCC2C3)cc1 ; c1ccc(-c2ccccc2-c2ccccc2-c2ccccc2N(c2ccc(-c3ccc4c(c3)oc3ccccc34)cc2)c2cccc3c2-c2ccccc2C32CCCC2)cc1]

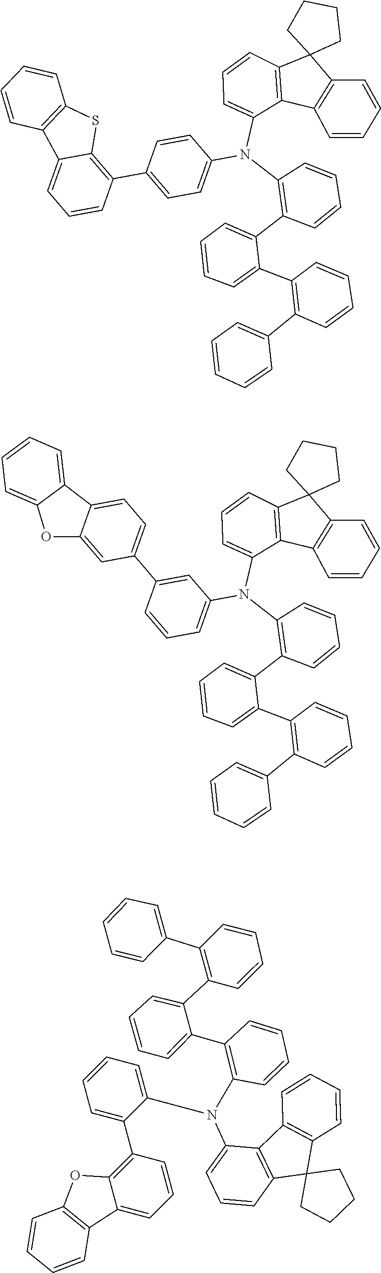 [SMILES: c1ccc(-c2ccccc2-c2ccccc2-c2ccccc2N(c2ccc(-c3cccc4c3sc3ccccc34)cc2)c2cccc3c2-c2ccccc2C32CCCC2)cc1 ; c1ccc(-c2ccccc2-c2ccccc2-c2ccccc2N(c2cccc(-c3ccc4c(c3)oc3ccccc34)c2)c2cccc3c2-c2ccccc2C32CCCC2)cc1 ; c1ccc(-c2ccccc2-c2ccccc2-c2ccccc2N(Cc2ccccc2-c2cccc3c2oc2ccccc23)c2cccc3c2-c2ccccc2C32CCCC2)cc1]

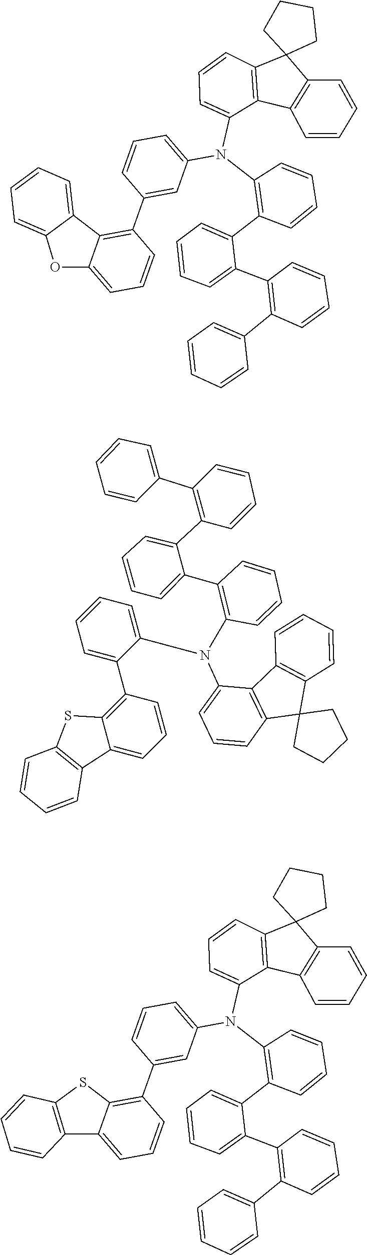 [SMILES: c1ccc(-c2ccccc2-c2ccccc2-c2ccccc2N(c2cccc(-c3cccc4oc5ccccc5c34)c2)c2cccc3c2-c2ccccc2C32CCCC2)cc1 ; c1ccc(-c2ccccc2-c2ccccc2-c2ccccc2N(Cc2ccccc2-c2cccc3c2sc2ccccc23)c2cccc3c2-c2ccccc2C32CCCC2)cc1 ; c1ccc(-c2ccccc2-c2ccccc2-c2ccccc2N(c2cccc(-c3cccc4c3sc3ccccc34)c2)c2cccc3c2-c2ccccc2C32CCCC2)cc1]

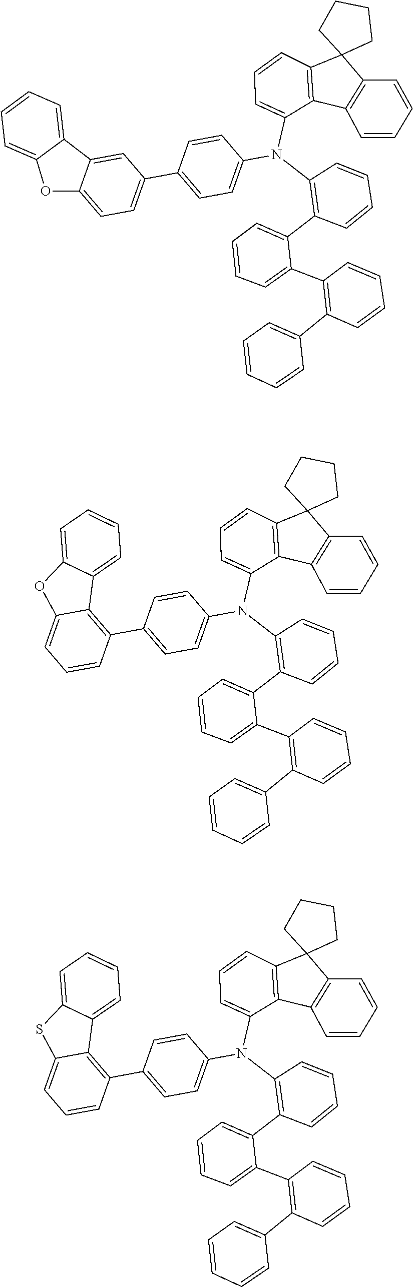 [SMILES: c1ccc(-c2ccccc2-c2ccccc2-c2ccccc2N(c2ccc(-c3ccc4oc5ccccc5c4c3)cc2)c2cccc3c2-c2ccccc2C32CCCC2)cc1 ; c1ccc(-c2ccccc2-c2ccccc2-c2ccccc2N(c2ccc(-c3cccc4oc5ccccc5c34)cc2)c2cccc3c2-c2ccccc2C32CCCC2)cc1 ; c1ccc(-c2ccccc2-c2ccccc2-c2ccccc2N(c2ccc(-c3cccc4sc5ccccc5c34)cc2)c2cccc3c2-c2ccccc2C32CCCC2)cc1]

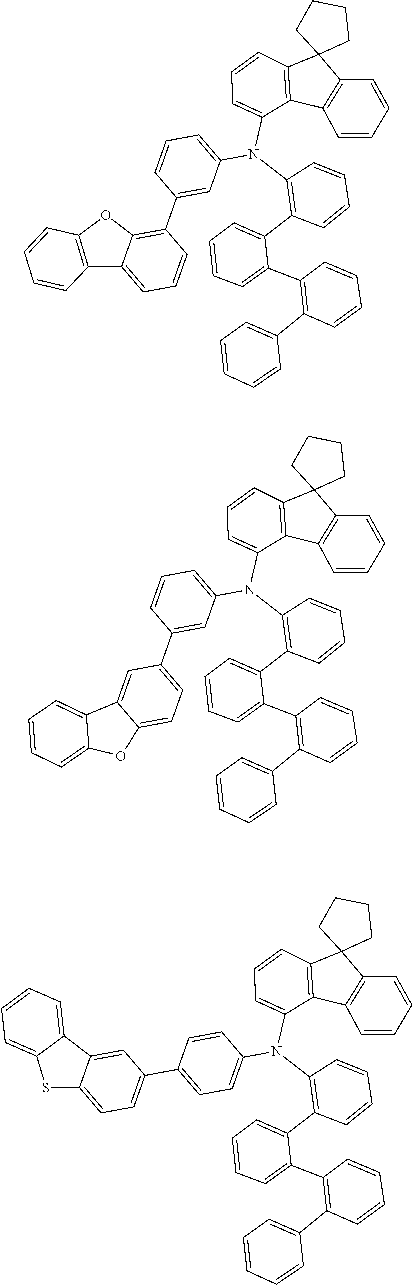 [SMILES: c1ccc(-c2ccccc2-c2ccccc2-c2ccccc2N(c2cccc(-c3cccc4c3oc3ccccc34)c2)c2cccc3c2-c2ccccc2C32CCCC2)cc1 ; c1ccc(-c2ccccc2-c2ccccc2-c2ccccc2N(c2cccc(-c3ccc4oc5ccccc5c4c3)c2)c2cccc3c2-c2ccccc2C32CCCC2)cc1 ; c1ccc(-c2ccccc2-c2ccccc2-c2ccccc2N(c2ccc(-c3ccc4sc5ccccc5c4c3)cc2)c2cccc3c2-c2ccccc2C32CCCC2)cc1]

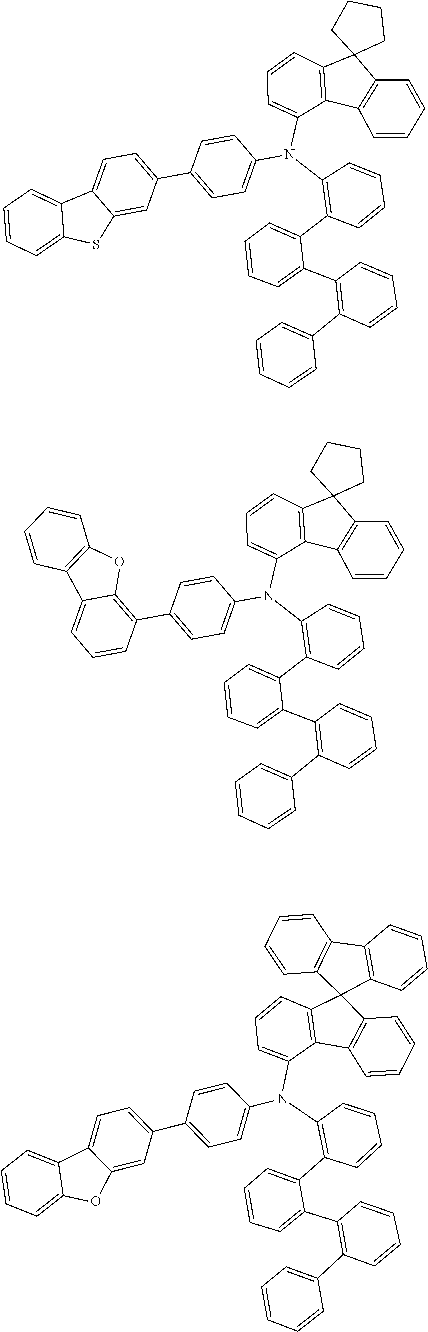 [SMILES: c1ccc(-c2ccccc2-c2ccccc2-c2ccccc2N(c2ccc(-c3ccc4c(c3)sc3ccccc34)cc2)c2cccc3c2-c2ccccc2C32CCCC2)cc1 ; c1ccc(-c2ccccc2-c2ccccc2-c2ccccc2N(c2ccc(-c3cccc4c3oc3ccccc34)cc2)c2cccc3c2-c2ccccc2C32CCCC2)cc1 ; c1ccc(-c2ccccc2-c2ccccc2-c2ccccc2N(c2ccc(-c3ccc4c(c3)oc3ccccc34)cc2)c2cccc3c2-c2ccccc2C32c3ccccc3-c3ccccc32)cc1]

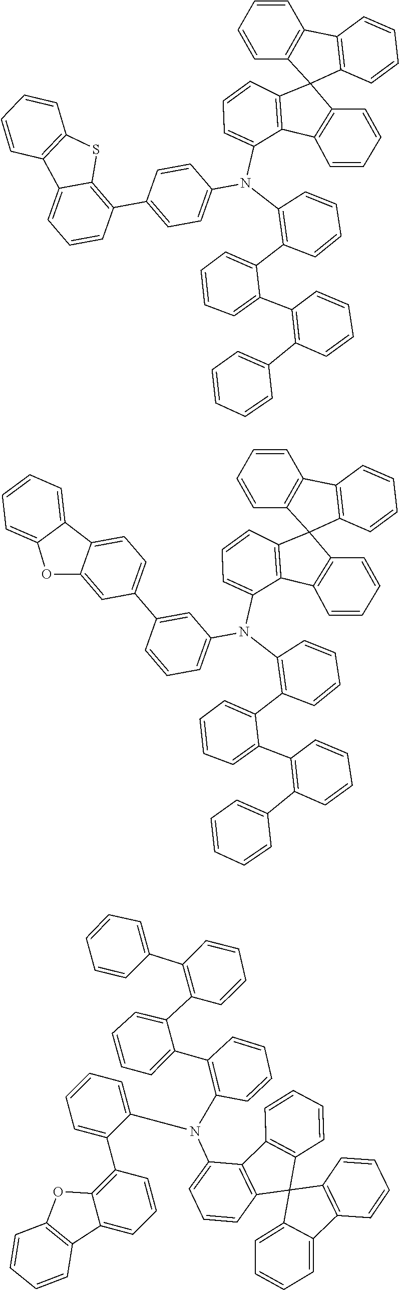

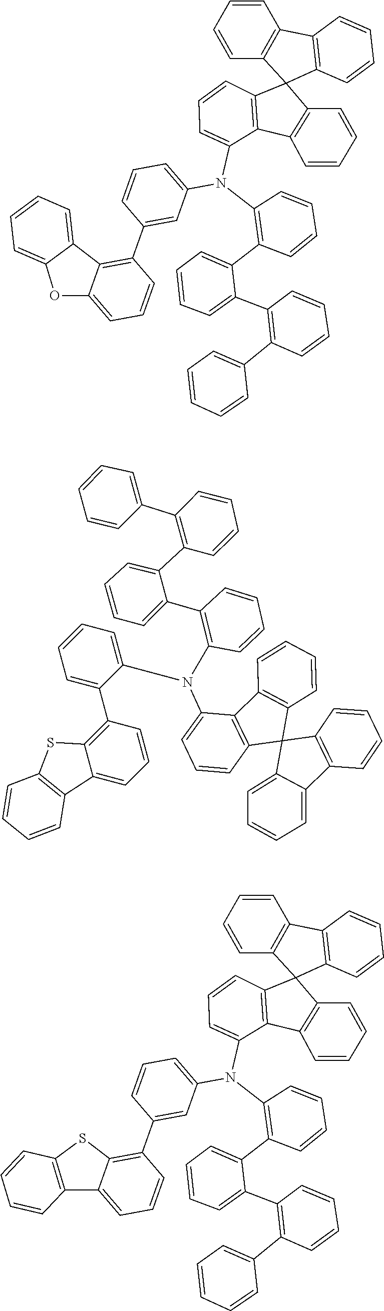 [SMILES: c1ccc(-c2ccccc2-c2ccccc2-c2ccccc2N(c2cccc(-c3cccc4oc5ccccc5c34)c2)c2cccc3c2-c2ccccc2C32c3ccccc3-c3ccccc32)cc1 ; c1ccc(-c2ccccc2-c2ccccc2-c2ccccc2N(Cc2ccccc2-c2cccc3c2sc2ccccc23)c2cccc3c2-c2ccccc2C32c3ccccc3-c3ccccc32)cc1 ; c1ccc(-c2ccccc2-c2ccccc2-c2ccccc2N(c2cccc(-c3cccc4c3sc3ccccc34)c2)c2cccc3c2-c2ccccc2C32c3ccccc3-c3ccccc32)cc1]

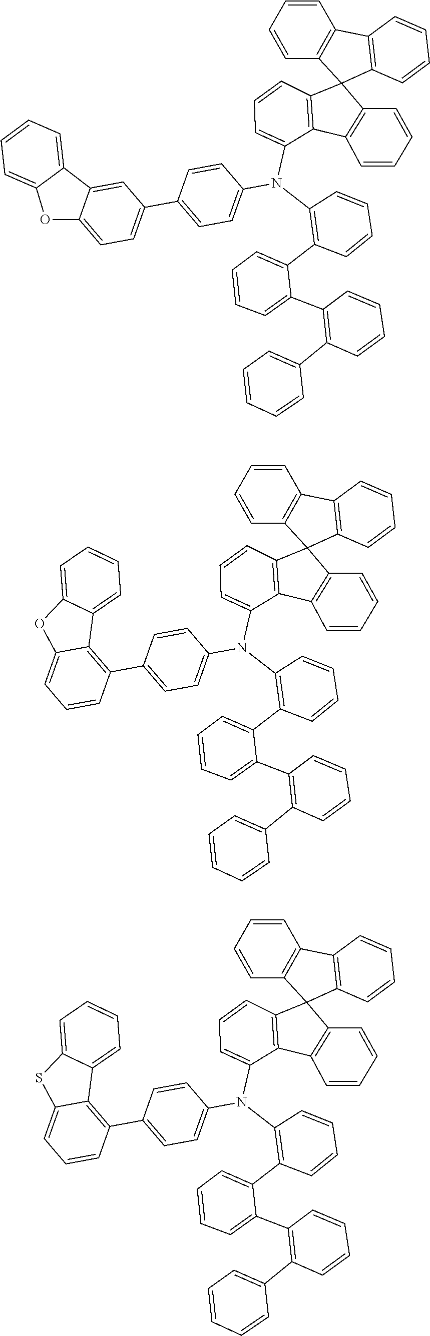

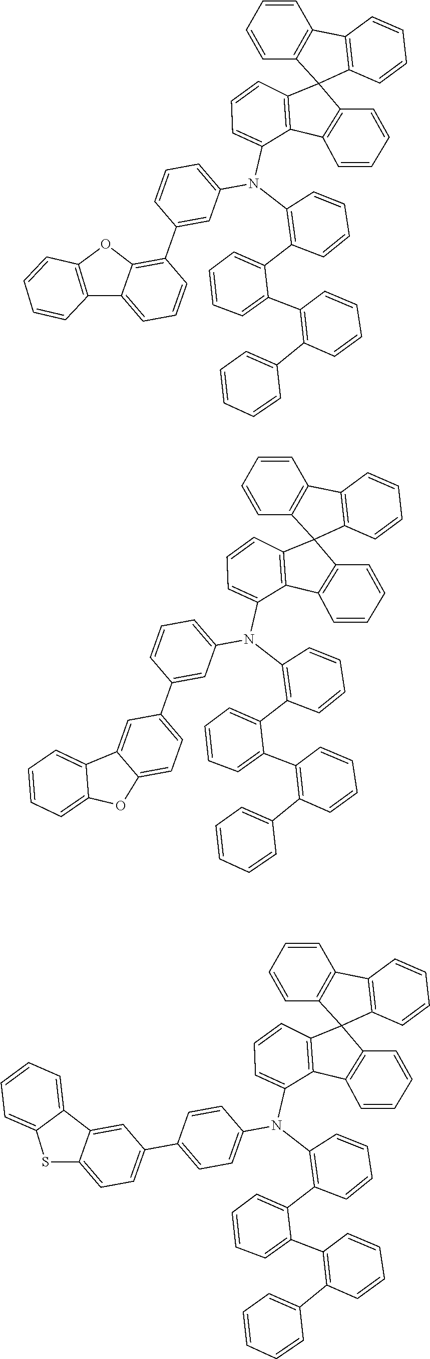

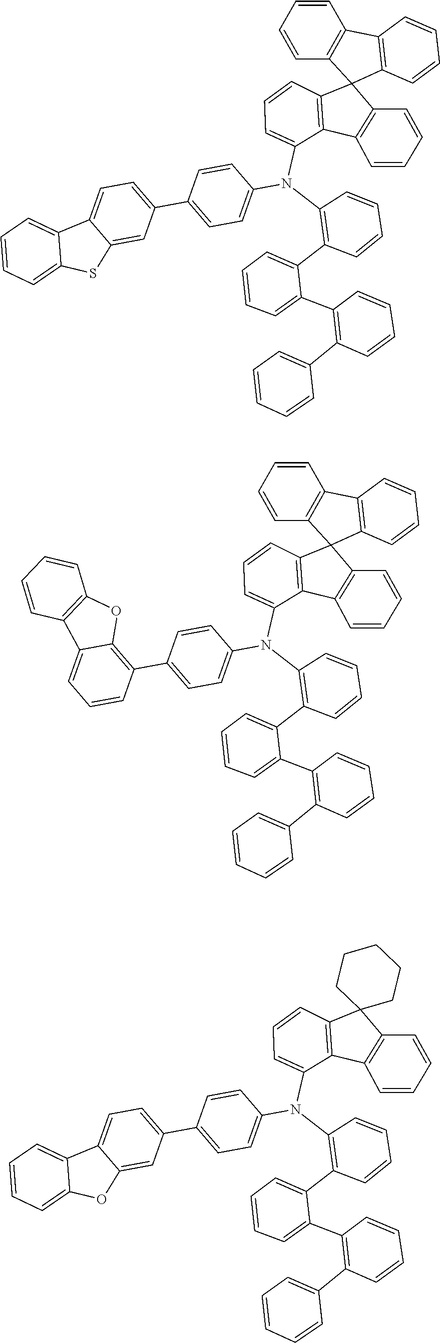 [SMILES: c1ccc(-c2ccccc2-c2ccccc2-c2ccccc2N(c2ccc(-c3ccc4c(c3)sc3ccccc34)cc2)c2cccc3c2-c2ccccc2C32c3ccccc3-c3ccccc32)cc1 ; c1ccc(-c2ccccc2-c2ccccc2-c2ccccc2N(c2ccc(-c3cccc4c3oc3ccccc34)cc2)c2cccc3c2-c2ccccc2C32c3ccccc3-c3ccccc32)cc1 ; c1ccc(-c2ccccc2-c2ccccc2-c2ccccc2N(c2ccc(-c3ccc4c(c3)oc3ccccc34)cc2)c2cccc3c2-c2ccccc2C32CCCCC2)cc1]

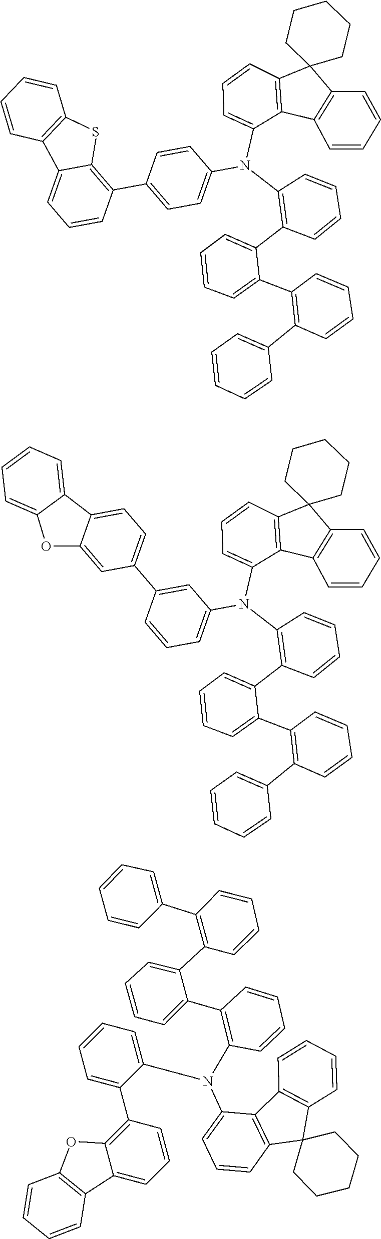 [SMILES: c1ccc(-c2ccccc2-c2ccccc2-c2ccccc2N(c2ccc(-c3cccc4c3sc3ccccc34)cc2)c2cccc3c2-c2ccccc2C32CCCCC2)cc1 ; c1ccc(-c2ccccc2-c2ccccc2-c2ccccc2N(c2cccc(-c3ccc4c(c3)oc3ccccc34)c2)c2cccc3c2-c2ccccc2C32CCCCC2)cc1 ; c1ccc(-c2ccccc2-c2ccccc2-c2ccccc2N(Cc2ccccc2-c2cccc3c2oc2ccccc23)c2cccc3c2-c2ccccc2C32CCCCC2)cc1]

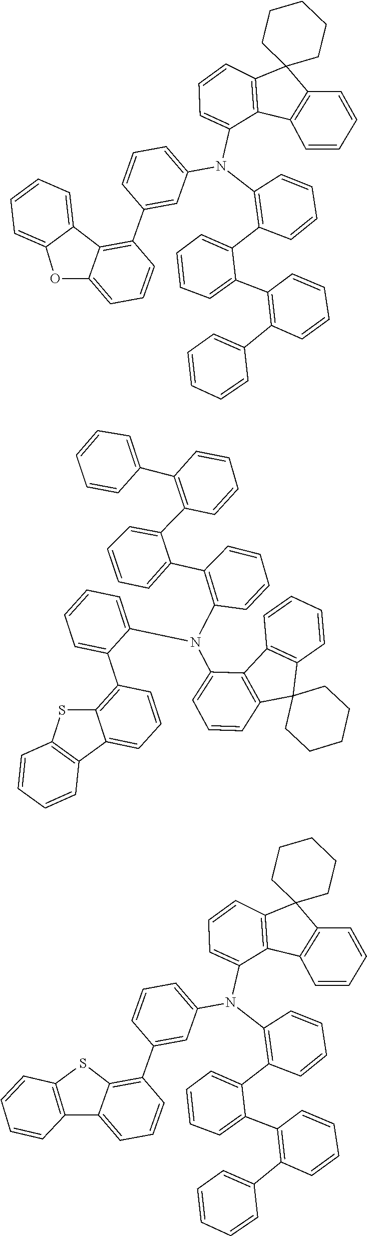 [SMILES: c1ccc(-c2ccccc2-c2ccccc2-c2ccccc2N(c2cccc(-c3cccc4oc5ccccc5c34)c2)c2cccc3c2-c2ccccc2C32CCCCC2)cc1 ; c1ccc(-c2ccccc2-c2ccccc2-c2ccccc2N(Cc2ccccc2-c2cccc3c2sc2ccccc23)c2cccc3c2-c2ccccc2C32CCCCC2)cc1 ; c1ccc(-c2ccccc2-c2ccccc2-c2ccccc2N(c2cccc(-c3cccc4c3sc3ccccc34)c2)c2cccc3c2-c2ccccc2C32CCCCC2)cc1]

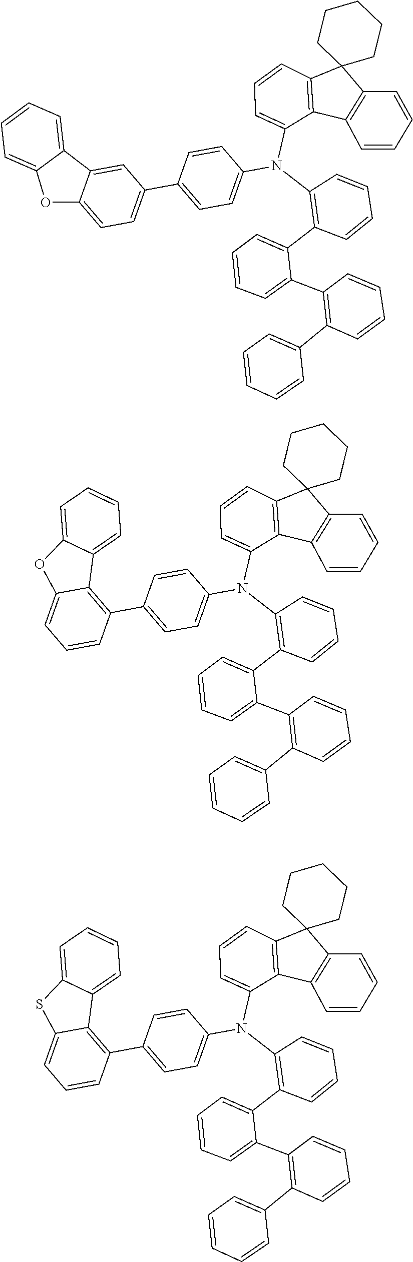

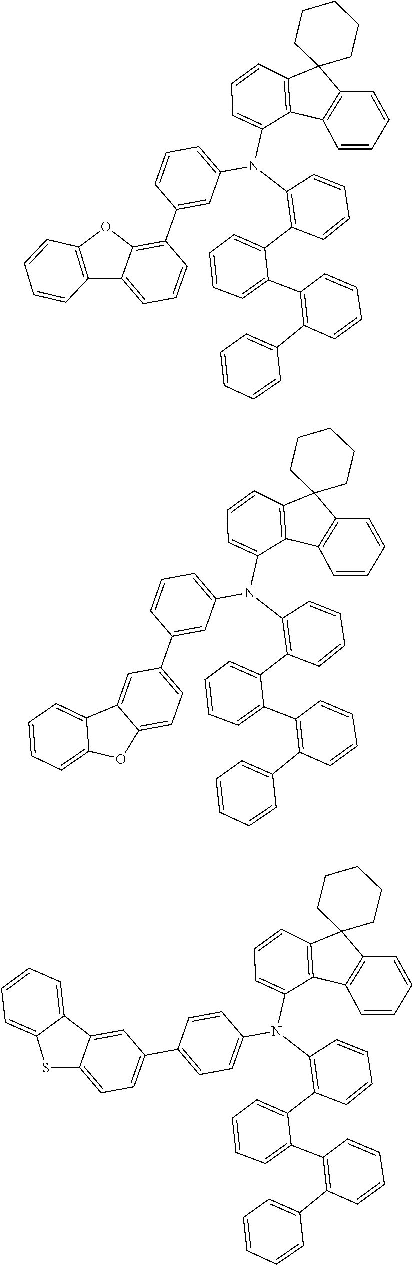 [SMILES: c1ccc(-c2ccccc2-c2ccccc2-c2ccccc2N(c2cccc(-c3cccc4c3oc3ccccc34)c2)c2cccc3c2-c2ccccc2C32CCCCC2)cc1 ; c1ccc(-c2ccccc2-c2ccccc2-c2ccccc2N(c2cccc(-c3ccc4oc5ccccc5c4c3)c2)c2cccc3c2-c2ccccc2C32CCCCC2)cc1 ; c1ccc(-c2ccccc2-c2ccccc2-c2ccccc2N(c2ccc(-c3ccc4sc5ccccc5c4c3)cc2)c2cccc3c2-c2ccccc2C32CCCCC2)cc1]

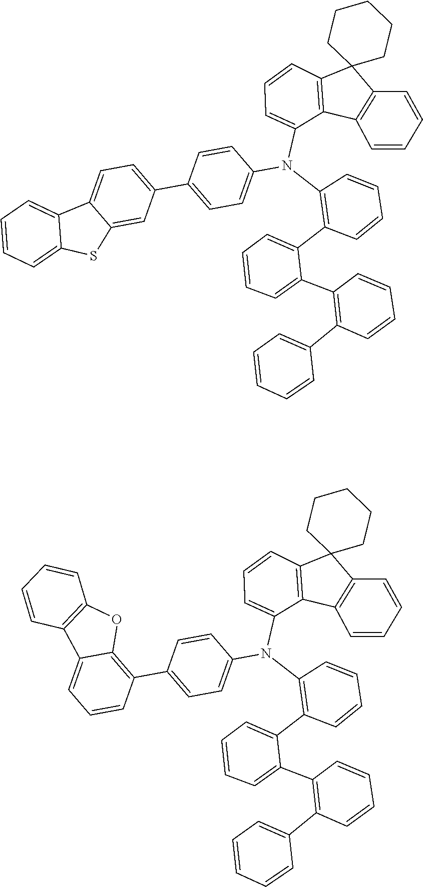 [SMILES: c1ccc(-c2ccccc2-c2ccccc2-c2ccccc2N(c2ccc(-c3ccc4c(c3)sc3ccccc34)cc2)c2cccc3c2-c2ccccc2C32CCCCC2)cc1 ; c1ccc(-c2ccccc2-c2ccccc2-c2ccccc2N(c2ccc(-c3cccc4c3oc3ccccc34)cc2)c2cccc3c2-c2ccccc2C32CCCCC2)cc1]

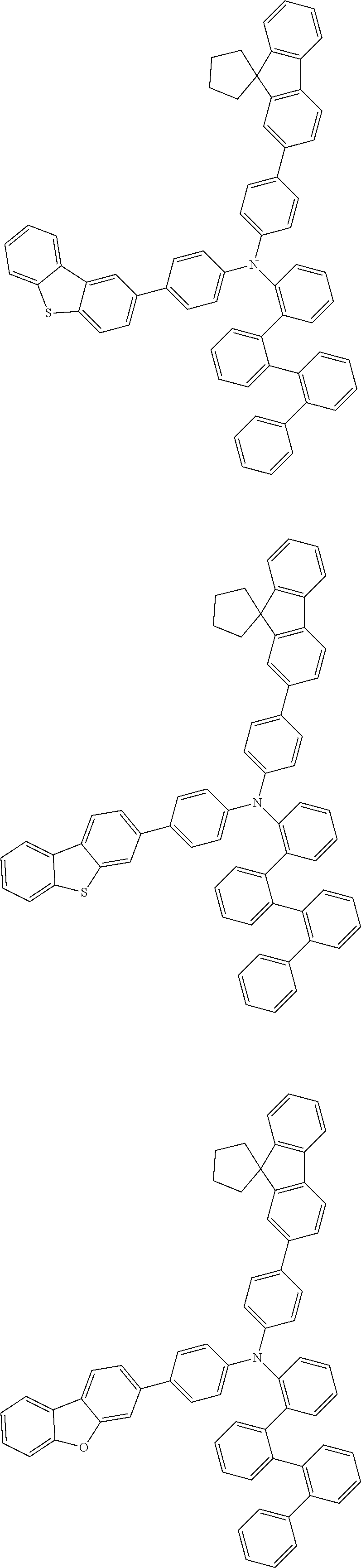 [SMILES: c1ccc(-c2ccccc2-c2ccccc2-c2ccccc2N(c2ccc(-c3ccc4c(c3)C3(CCCC3)c3ccccc3-4)cc2)c2ccc(-c3ccc4sc5ccccc5c4c3)cc2)cc1 ; c1ccc(-c2ccccc2-c2ccccc2-c2ccccc2N(c2ccc(-c3ccc4c(c3)C3(CCCC3)c3ccccc3-4)cc2)c2ccc(-c3ccc4c(c3)sc3ccccc34)cc2)cc1 ; c1ccc(-c2ccccc2-c2ccccc2-c2ccccc2N(c2ccc(-c3ccc4c(c3)C3(CCCC3)c3ccccc3-4)cc2)c2ccc(-c3ccc4c(c3)oc3ccccc34)cc2)cc1]

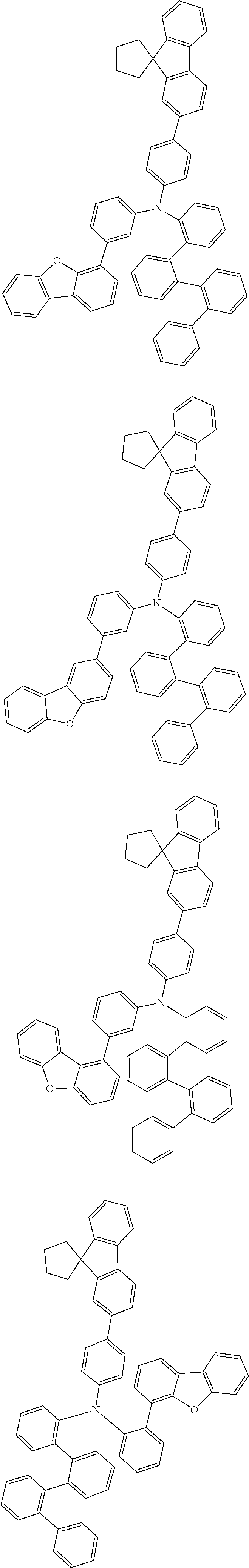

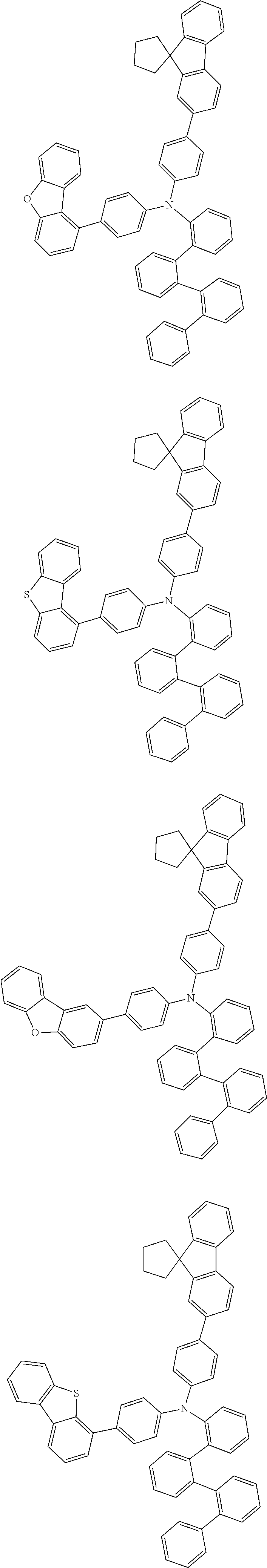 [SMILES: c1ccc(-c2ccccc2-c2ccccc2-c2ccccc2N(c2ccc(-c3ccc4c(c3)C3(CCCC3)c3ccccc3-4)cc2)c2ccc(-c3cccc4c3Cc3ccccc3O4)cc2)cc1 ; c1ccc(-c2ccccc2-c2ccccc2-c2ccccc2N(c2ccc(-c3ccc4c(c3)C3(CCCC3)c3ccccc3-4)cc2)c2ccc(-c3cccc4c3Cc3ccccc3S4)cc2)cc1 ; c1ccc(-c2ccccc2-c2ccccc2-c2ccccc2N(c2ccc(-c3ccc4c(c3)C3(CCCC3)c3ccccc3-4)cc2)c2ccc(-c3ccc4oc5ccccc5c4c3)cc2)cc1 ; c1ccc(-c2ccccc2-c2ccccc2-c2ccccc2N(c2ccc(-c3ccc4c(c3)C3(CCCC3)c3ccccc3-4)cc2)c2ccc(-c3cccc4c3sc3ccccc34)cc2)cc1]

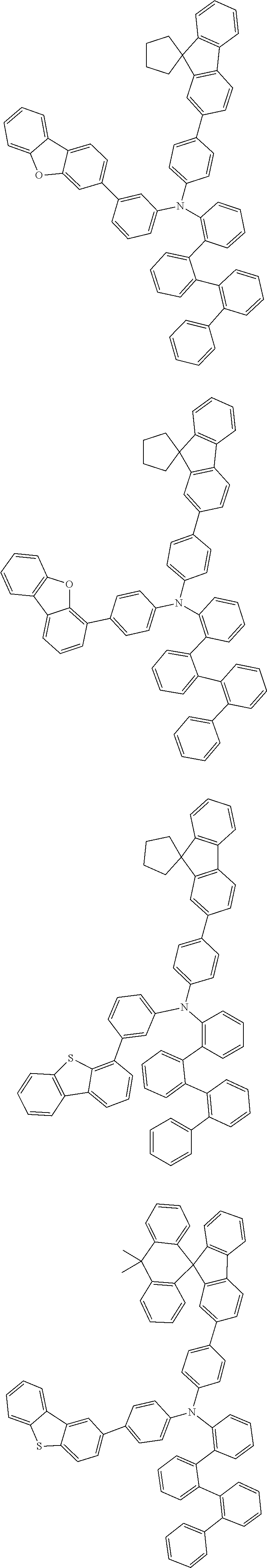 [SMILES: c1ccc(-c2ccccc2-c2ccccc2-c2ccccc2N(c2ccc(-c3ccc4c(c3)C3(CCCC3)c3ccccc3-4)cc2)c2cccc(-c3ccc4c(c3)oc3ccccc34)c2)cc1 ; c1ccc(-c2ccccc2-c2ccccc2-c2ccccc2N(c2ccc(-c3ccc4c(c3)C3(CCCC3)c3ccccc3-4)cc2)c2ccc(-c3cccc4c3oc3ccccc34)cc2)cc1 ; c1ccc(-c2ccccc2-c2ccccc2-c2ccccc2N(c2ccc(-c3ccc4c(c3)C3(CCCC3)c3ccccc3-4)cc2)c2cccc(-c3cccc4c3sc3ccccc34)c2)cc1 ; CC1(C)c2ccccc2C2(c3ccccc3-c3ccc(-c4ccc(N(c5ccc(-c6ccc7sc8ccccc8c7c6)cc5)c5ccccc5-c5ccccc5-c5ccccc5-c5ccccc5)cc4)cc32)c2ccccc21]

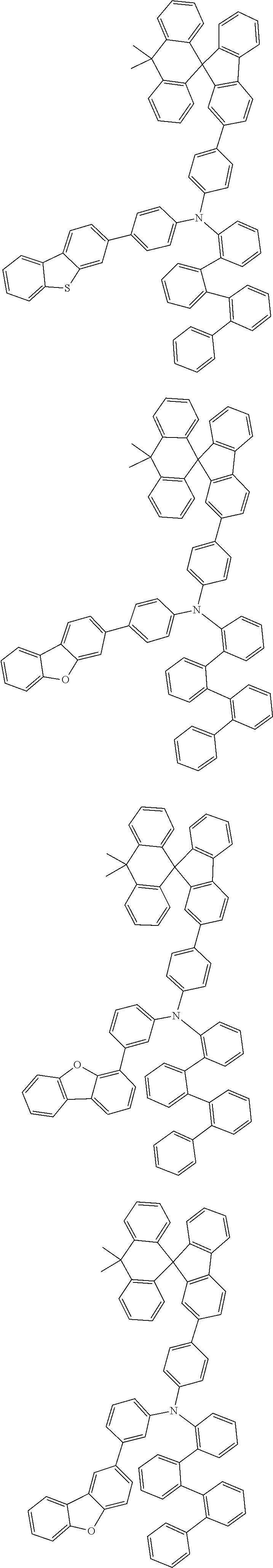

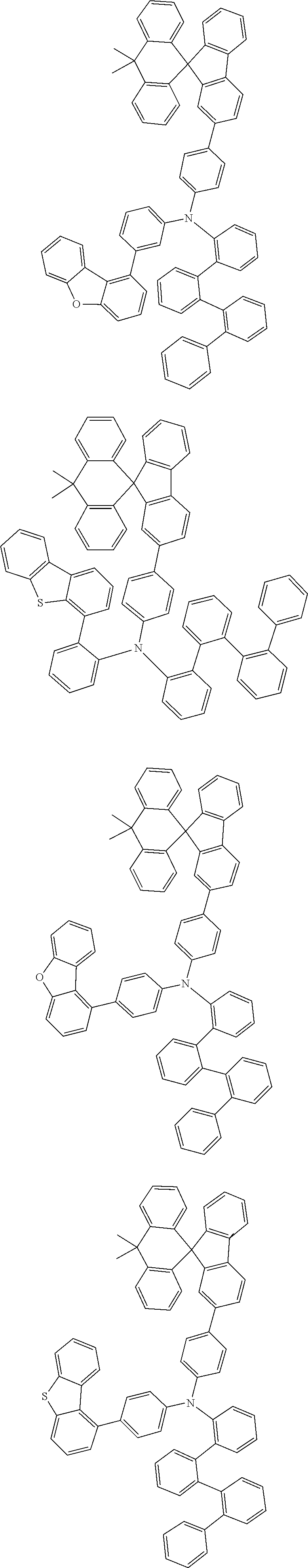

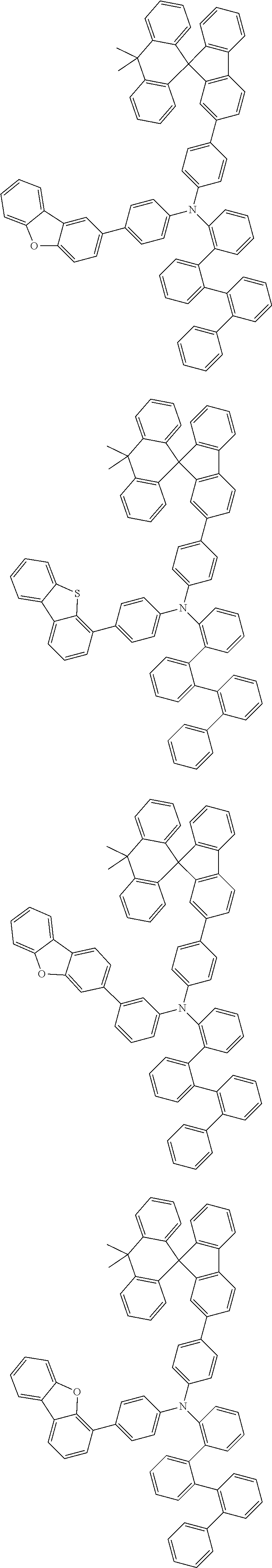

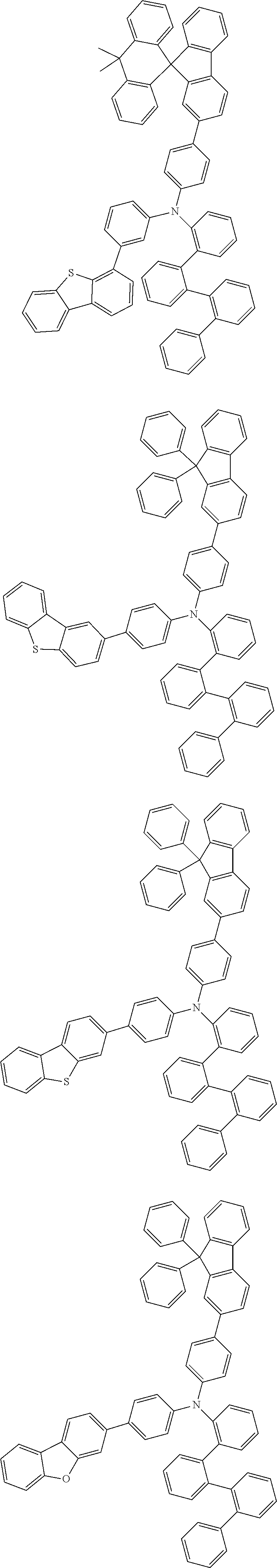

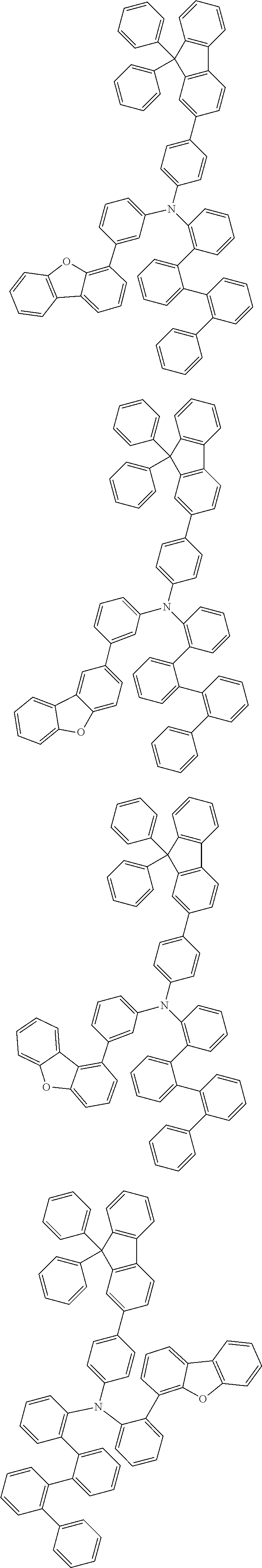

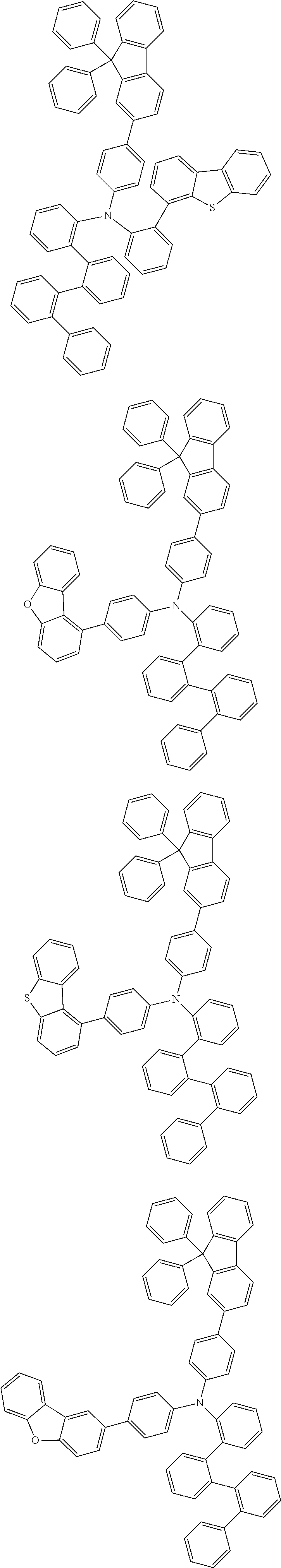

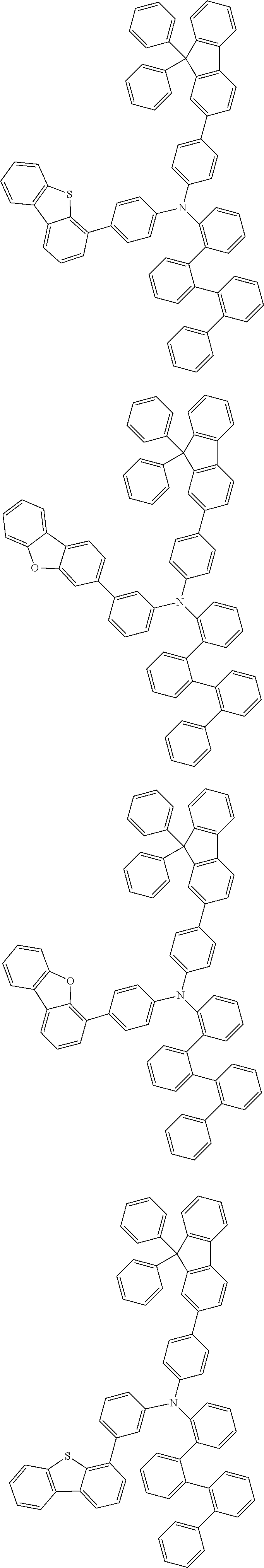

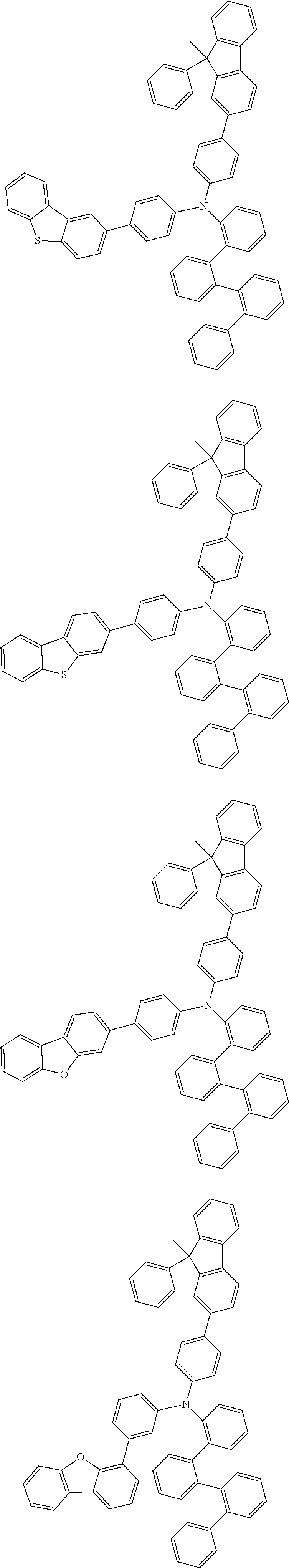

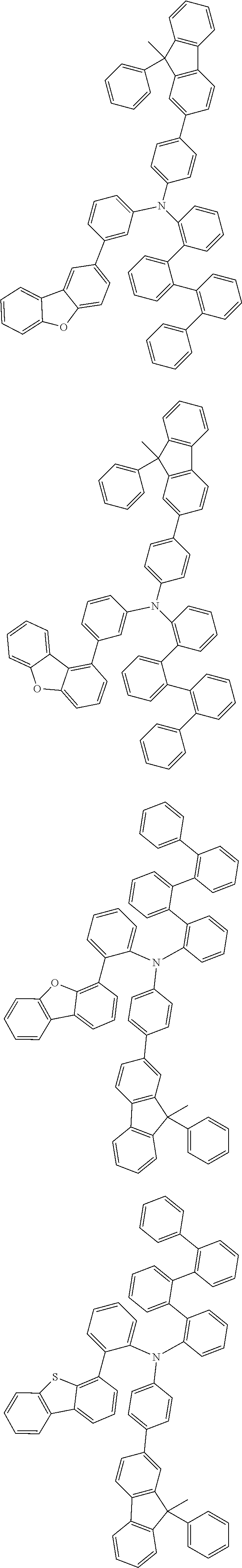

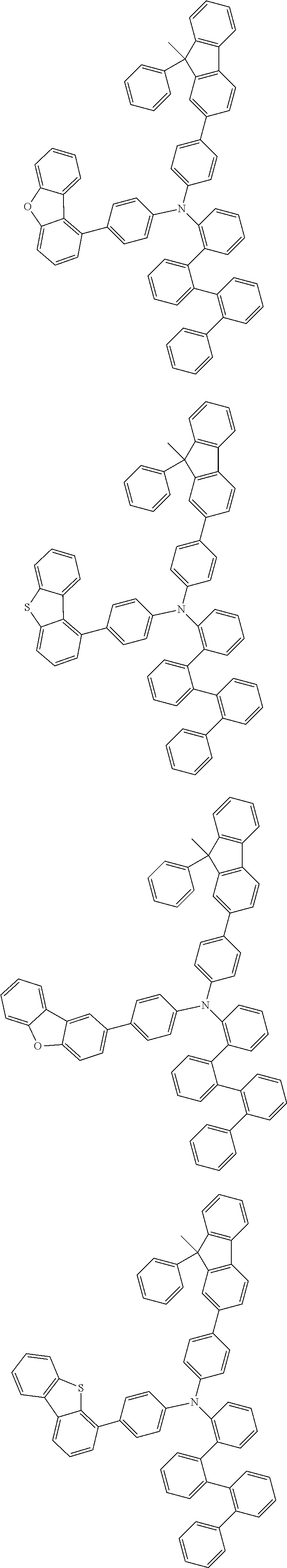

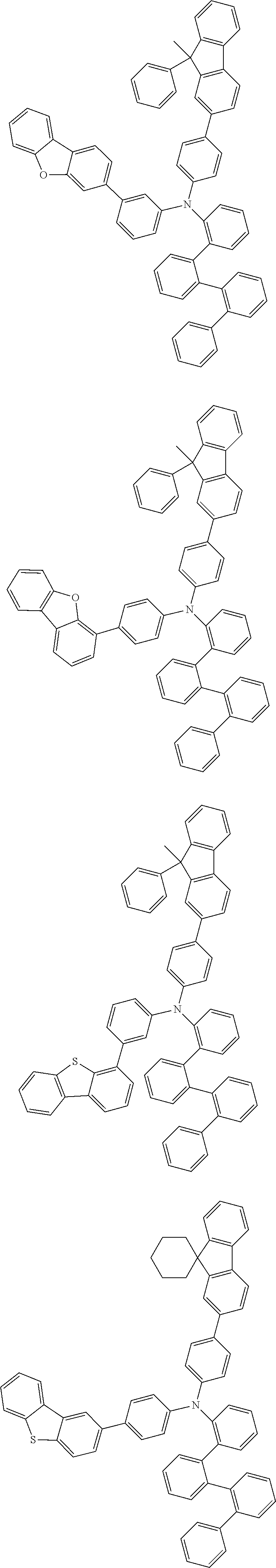 [SMILES: CC1(c2ccccc2)c2ccccc2-c2ccc(-c3ccc(N(c4cccc(-c5ccc6c(c5)oc5ccccc56)c4)c4ccccc4-c4ccccc4-c4ccccc4-c4ccccc4)cc3)cc21 ; CC1(c2ccccc2)c2ccccc2-c2ccc(-c3ccc(N(c4ccc(-c5cccc6c5oc5ccccc56)cc4)c4ccccc4-c4ccccc4-c4ccccc4-c4ccccc4)cc3)cc21 ; CC1(c2ccccc2)c2ccccc2-c2ccc(-c3ccc(N(c4cccc(-c5cccc6c5sc5ccccc56)c4)c4ccccc4-c4ccccc4-c4ccccc4-c4ccccc4)cc3)cc21 ; c1ccc(-c2ccccc2-c2ccccc2-c2ccccc2N(c2ccc(-c3ccc4c(c3)C3(CCCCC3)c3ccccc3-4)cc2)c2ccc(-c3ccc4sc5ccccc5c4c3)cc2)cc1]

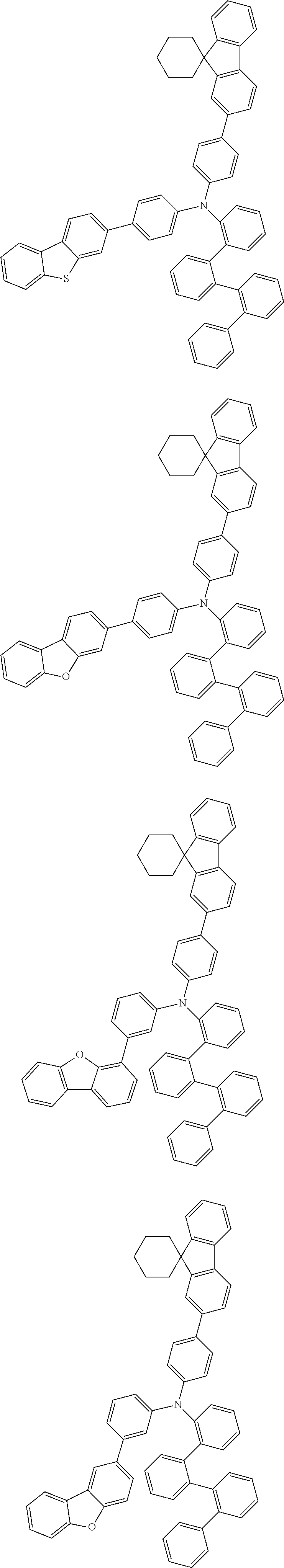

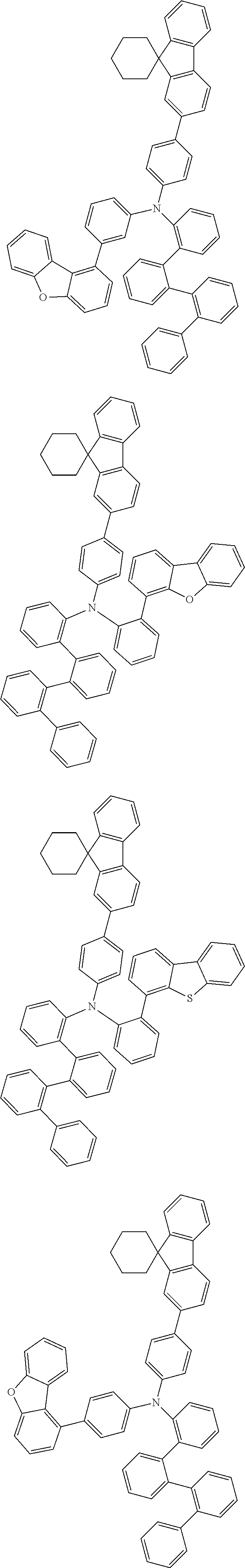 [SMILES: c1ccc(-c2ccccc2-c2ccccc2-c2ccccc2N(c2ccc(-c3ccc4c(c3)C3(CCCCC3)c3ccccc3-4)cc2)c2cccc(-c3cccc4oc5ccccc5c34)c2)cc1 ; c1ccc(-c2ccccc2-c2ccccc2-c2ccccc2N(c2ccc(-c3ccc4c(c3)C3(CCCCC3)c3ccccc3-4)cc2)c2ccccc2-c2cccc3c2oc2ccccc23)cc1 ; c1ccc(-c2ccccc2-c2ccccc2-c2ccccc2N(c2ccc(-c3ccc4c(c3)C3(CCCCC3)c3ccccc3-4)cc2)c2ccccc2-c2cccc3c2sc2ccccc23)cc1 ; c1ccc(-c2ccccc2-c2ccccc2-c2ccccc2N(c2ccc(-c3ccc4c(c3)C3(CCCCC3)c3ccccc3-4)cc2)c2ccc(-c3cccc4oc5ccccc5c34)cc2)cc1]

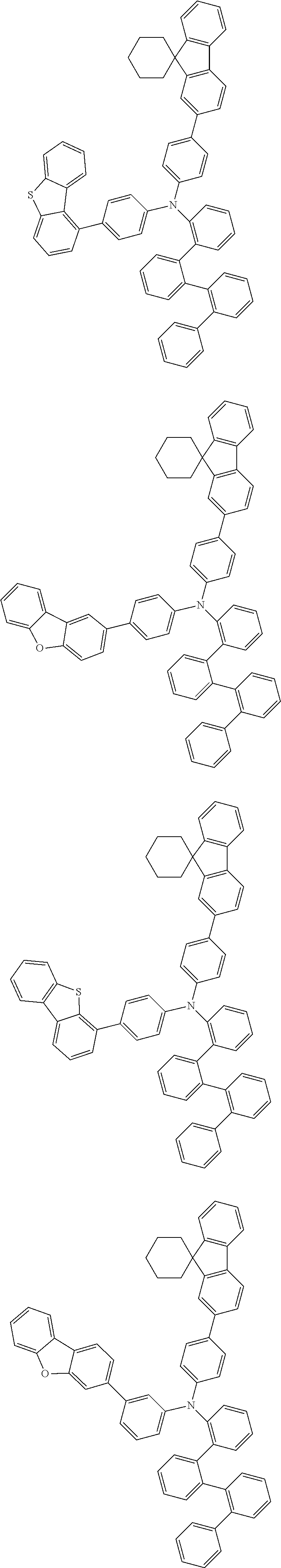 [SMILES: c1ccc(-c2ccccc2-c2ccccc2-c2ccccc2N(c2ccc(-c3ccc4c(c3)C3(CCCCC3)c3ccccc3-4)cc2)c2ccc(-c3cccc4sc5ccccc5c34)cc2)cc1 ; c1ccc(-c2ccccc2-c2ccccc2-c2ccccc2N(c2ccc(-c3ccc4c(c3)C3(CCCCC3)c3ccccc3-4)cc2)c2ccc(-c3ccc4oc5ccccc5c4c3)cc2)cc1 ; c1ccc(-c2ccccc2-c2ccccc2-c2ccccc2N(c2ccc(-c3ccc4c(c3)C3(CCCCC3)c3ccccc3-4)cc2)c2ccc(-c3cccc4c3sc3ccccc34)cc2)cc1 ; c1ccc(-c2ccccc2-c2ccccc2-c2ccccc2N(c2ccc(-c3ccc4c(c3)C3(CCCCC3)c3ccccc3-4)cc2)c2cccc(-c3ccc4c(c3)oc3ccccc34)c2)cc1]

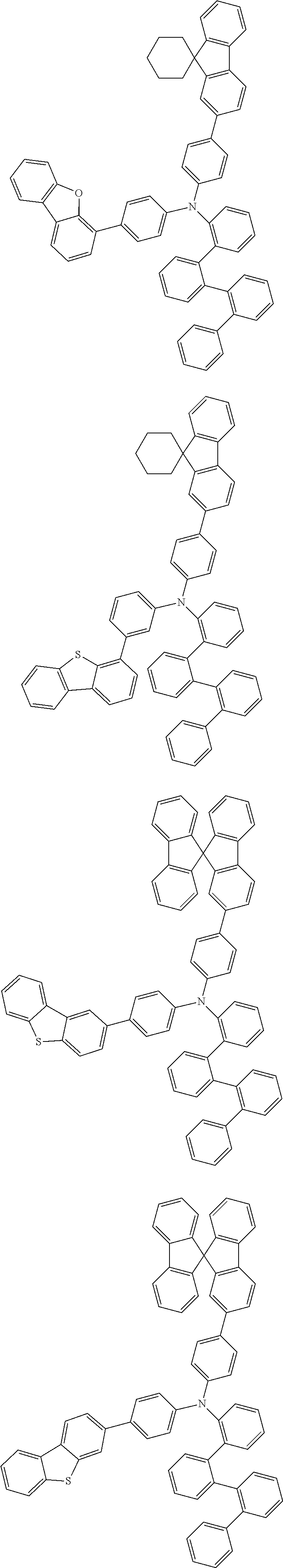

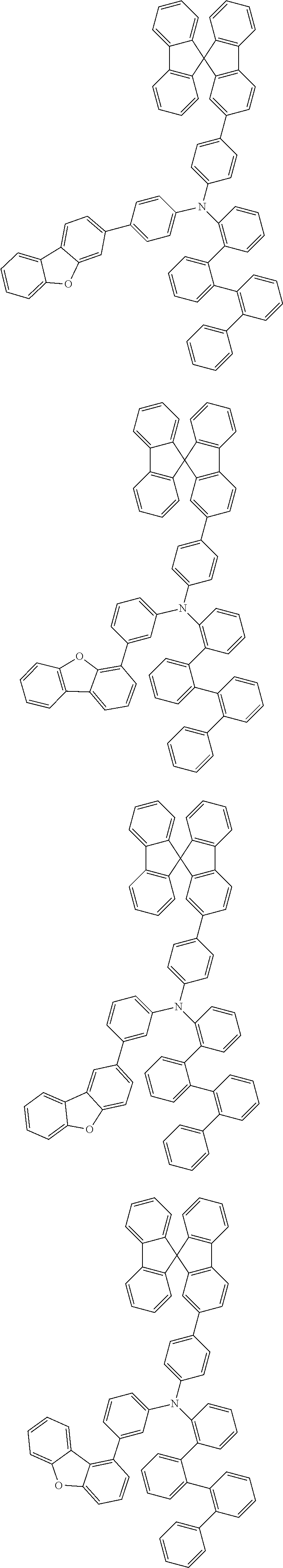

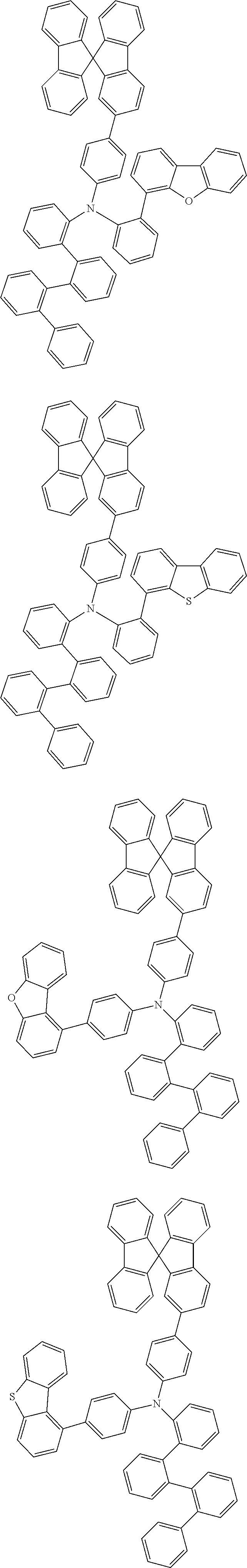

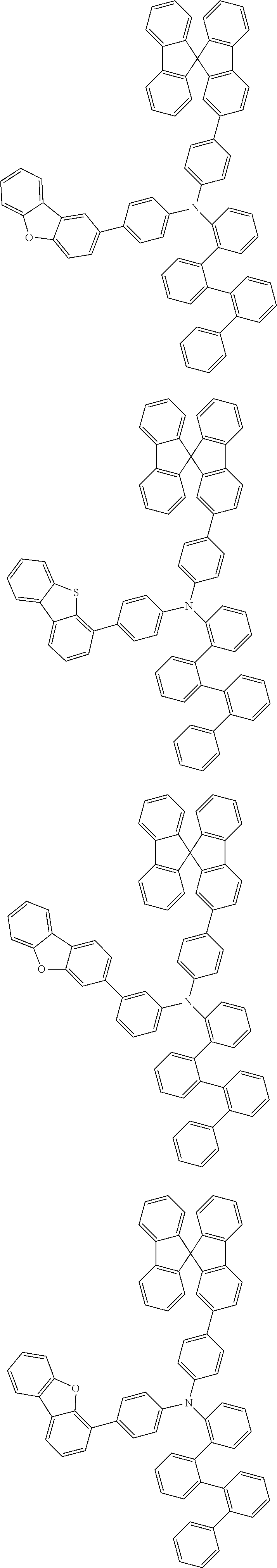

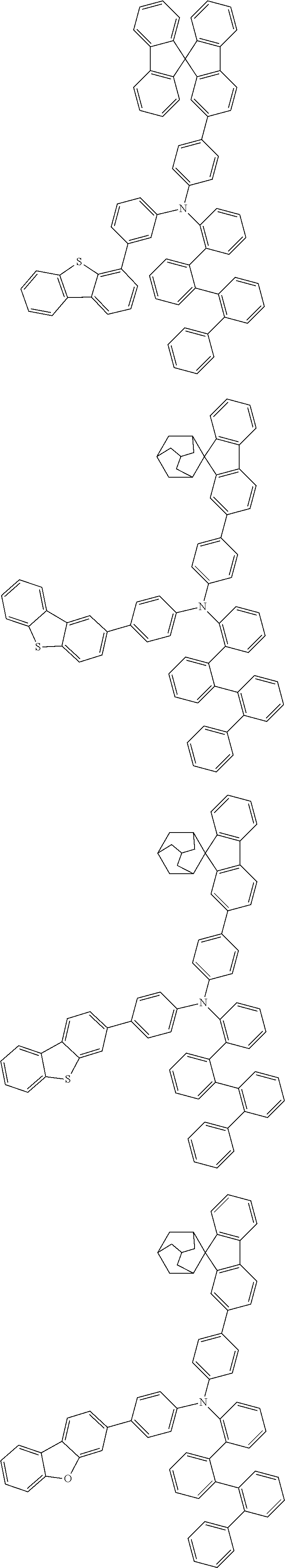

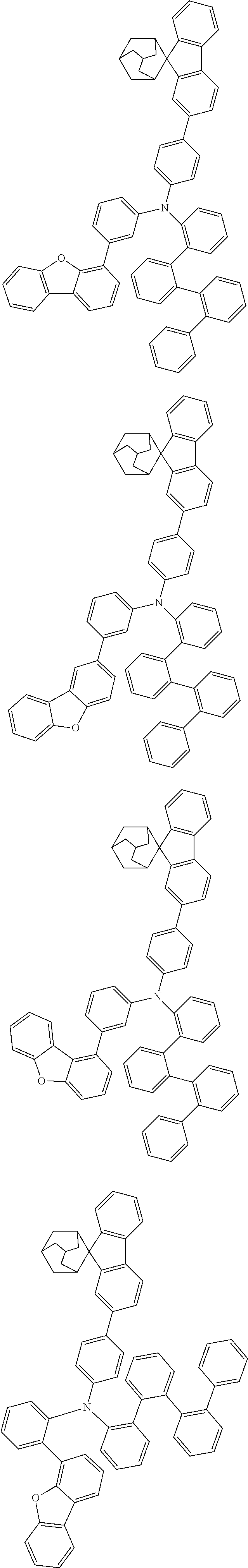

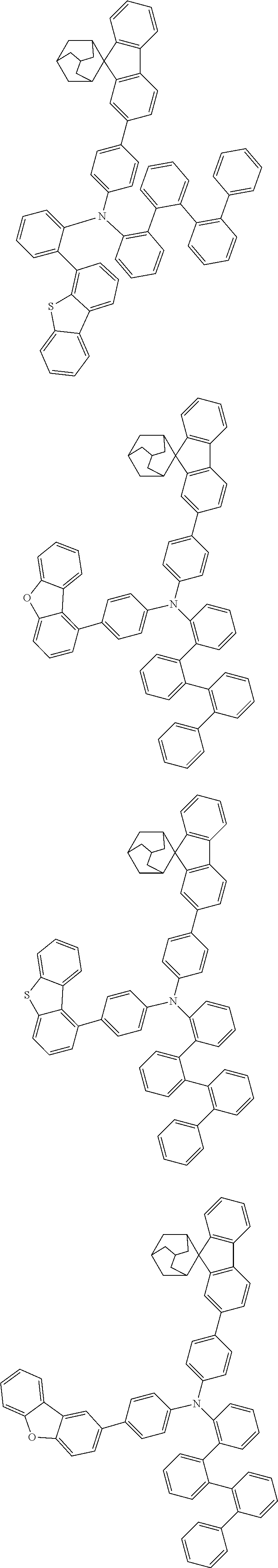

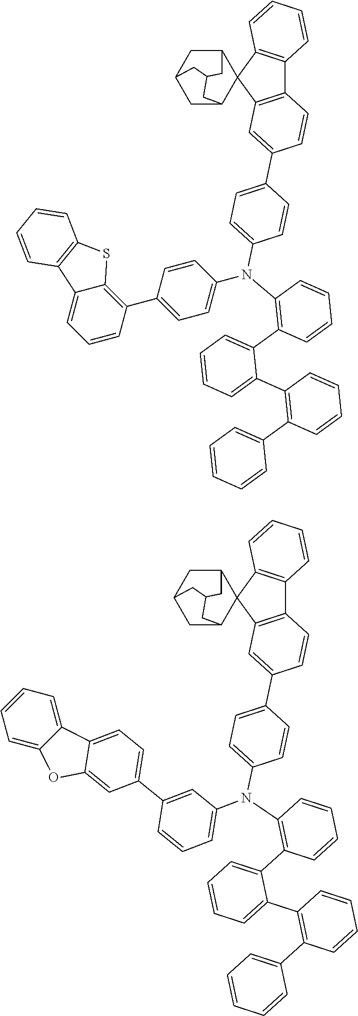 [SMILES: c1ccc(-c2ccccc2-c2ccccc2-c2ccccc2N(c2ccc(-c3ccc4c(c3)C3(c5ccccc5-4)C4CC5CC(C4)CC3C5)cc2)c2ccc(-c3cccc4c3sc3ccccc34)cc2)cc1 ; c1ccc(-c2ccccc2-c2ccccc2-c2ccccc2N(c2ccc(-c3ccc4c(c3)C3(c5ccccc5-4)C4CC5CC(C4)CC3C5)cc2)c2cccc(-c3ccc4c(c3)oc3ccccc34)c2)cc1]

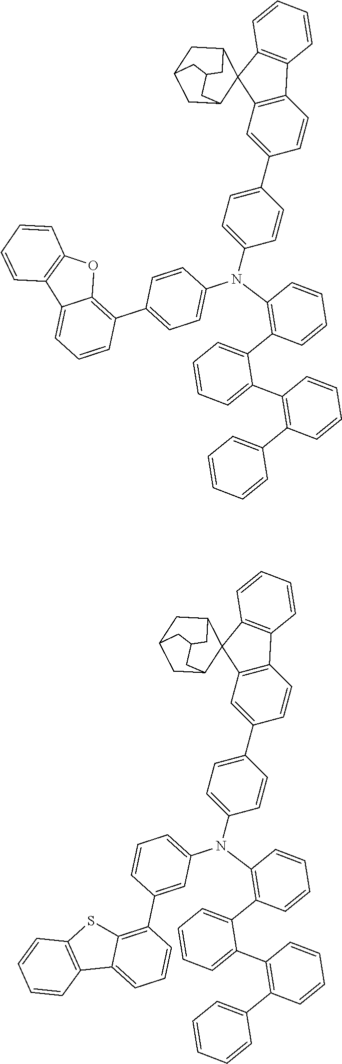 [SMILES: c1ccc(-c2ccccc2-c2ccccc2-c2ccccc2N(c2ccc(-c3ccc4c(c3)C3(c5ccccc5-4)C4CC5CC(C4)CC3C5)cc2)c2ccc(-c3cccc4c3oc3ccccc34)cc2)cc1 ; c1ccc(-c2ccccc2-c2ccccc2-c2ccccc2N(c2ccc(-c3ccc4c(c3)C3(c5ccccc5-4)C4CC5CC(C4)CC3C5)cc2)c2cccc(-c3cccc4c3sc3ccccc34)c2)cc1]

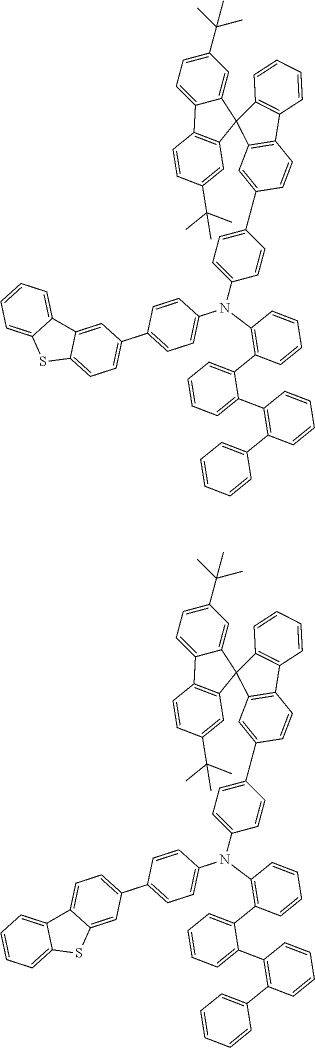 [SMILES: CC(C)(C)c1ccc2c(c1)C1(c3ccccc3-c3ccc(-c4ccc(N(c5ccc(-c6ccc7sc8ccccc8c7c6)cc5)c5ccccc5-c5ccccc5-c5ccccc5-c5ccccc5)cc4)cc31)c1cc(C(C)(C)C)ccc1-2 ; CC(C)(C)c1ccc2c(c1)C1(c3ccccc3-c3ccc(-c4ccc(N(c5ccc(-c6ccc7c(c6)sc6ccccc67)cc5)c5ccccc5-c5ccccc5-c5ccccc5-c5ccccc5)cc4)cc31)c1cc(C(C)(C)C)ccc1-2]

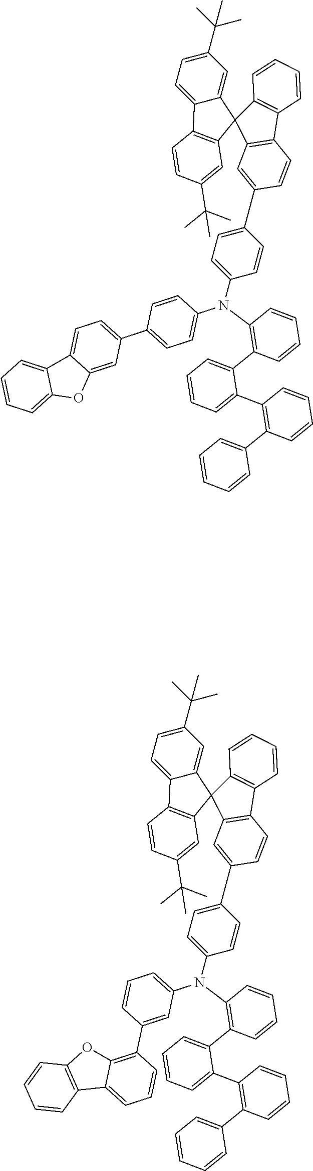 [SMILES: CC(C)(C)c1ccc2c(c1)C1(c3ccccc3-c3ccc(-c4ccc(N(c5ccc(-c6ccc7c(c6)oc6ccccc67)cc5)c5ccccc5-c5ccccc5-c5ccccc5-c5ccccc5)cc4)cc31)c1cc(C(C)(C)C)ccc1-2 ; CC(C)(C)c1ccc2c(c1)C1(c3ccccc3-c3ccc(-c4ccc(N(c5cccc(-c6cccc7c6oc6ccccc67)c5)c5ccccc5-c5ccccc5-c5ccccc5-c5ccccc5)cc4)cc31)c1cc(C(C)(C)C)ccc1-2]

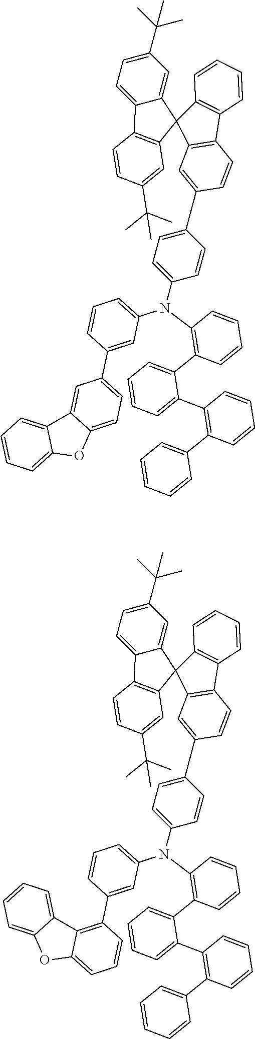 [SMILES: CC(C)(C)c1ccc2c(c1)C1(c3ccccc3-c3ccc(-c4ccc(N(c5cccc(-c6ccc7oc8ccccc8c7c6)c5)c5ccccc5-c5ccccc5-c5ccccc5-c5ccccc5)cc4)cc31)c1cc(C(C)(C)C)ccc1-2 ; CC(C)(C)c1ccc2c(c1)C1(c3ccccc3-c3ccc(-c4ccc(N(c5cccc(-c6cccc7oc8ccccc8c67)c5)c5ccccc5-c5ccccc5-c5ccccc5-c5ccccc5)cc4)cc31)c1cc(C(C)(C)C)ccc1-2]

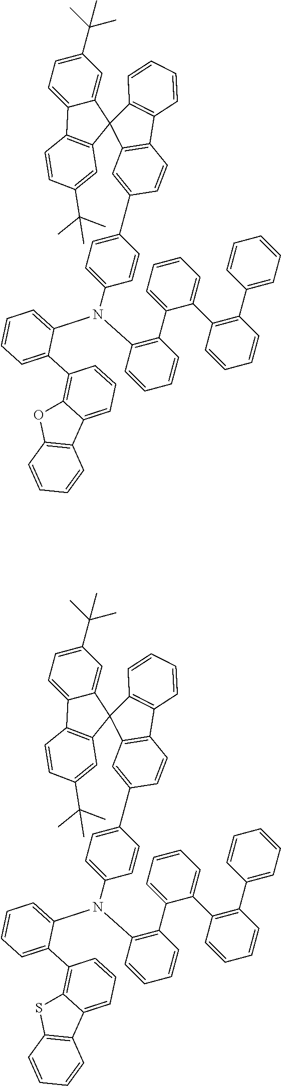 [SMILES: CC(C)(C)c1ccc2c(c1)C1(c3ccccc3-c3ccc(-c4ccc(N(c5ccccc5-c5ccccc5-c5ccccc5-c5ccccc5)c5ccccc5-c5cccc6c5oc5ccccc56)cc4)cc31)c1cc(C(C)(C)C)ccc1-2 ; CC(C)(C)c1ccc2c(c1)C1(c3ccccc3-c3ccc(-c4ccc(N(c5ccccc5-c5ccccc5-c5ccccc5-c5ccccc5)c5ccccc5-c5cccc6c5sc5ccccc56)cc4)cc31)c1cc(C(C)(C)C)ccc1-2]

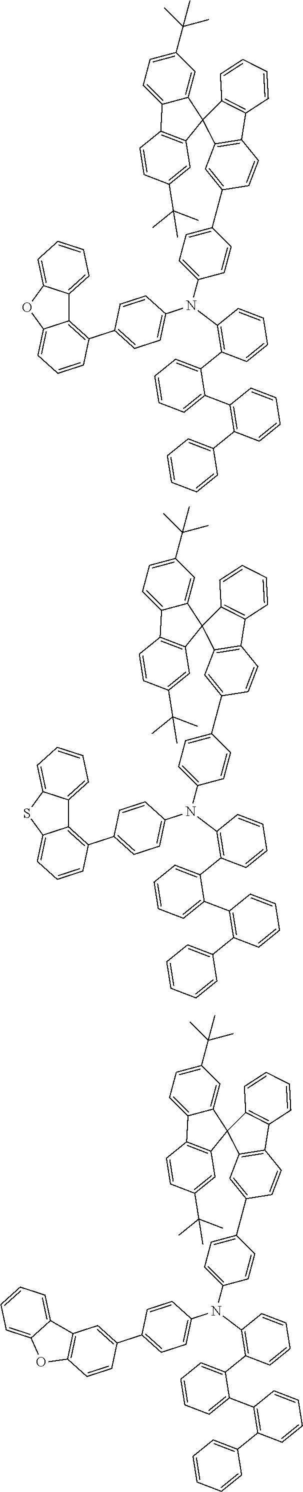

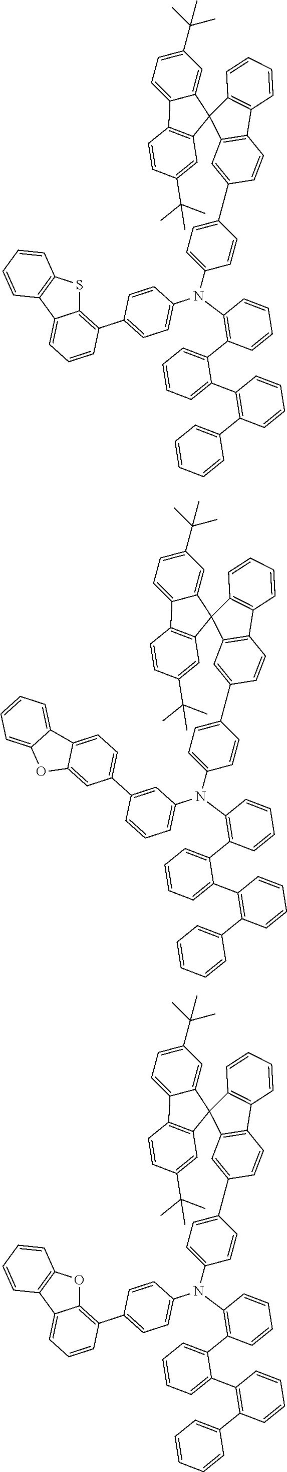

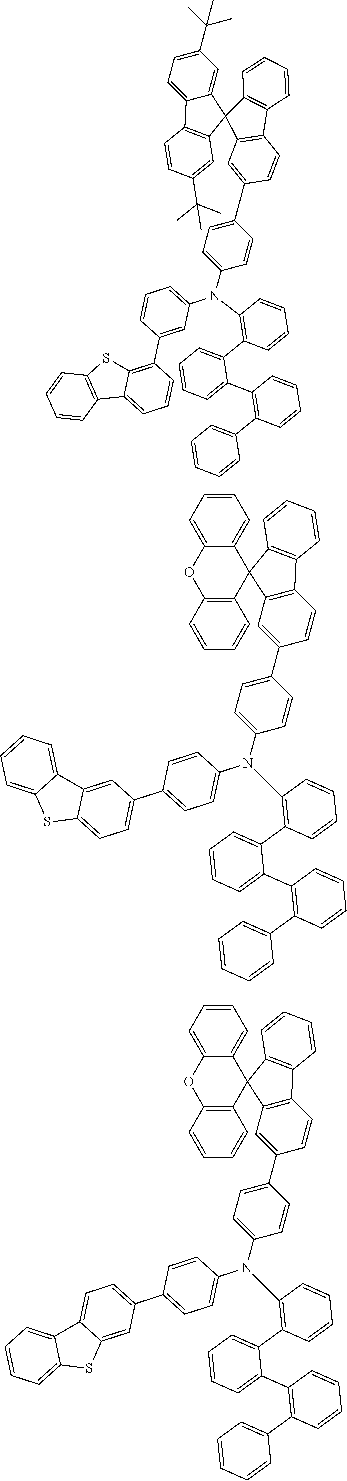

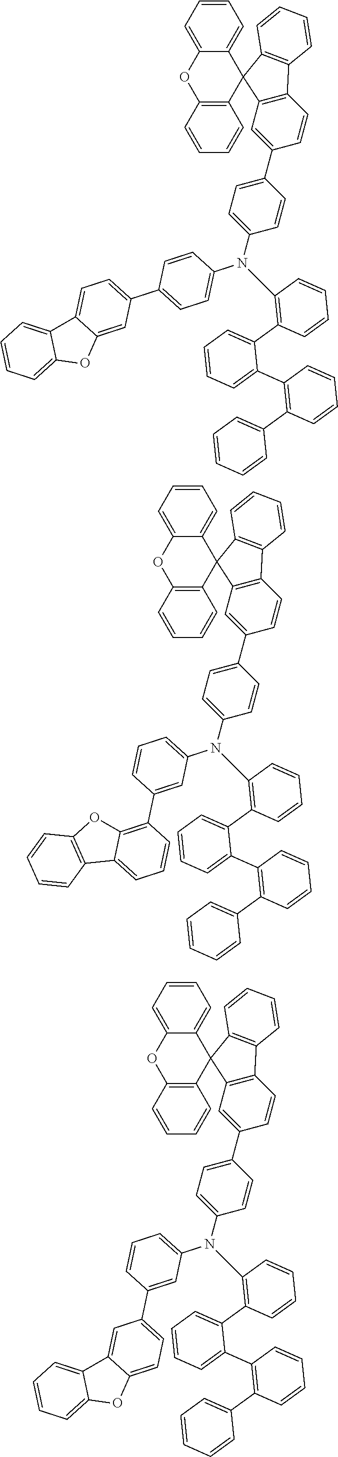

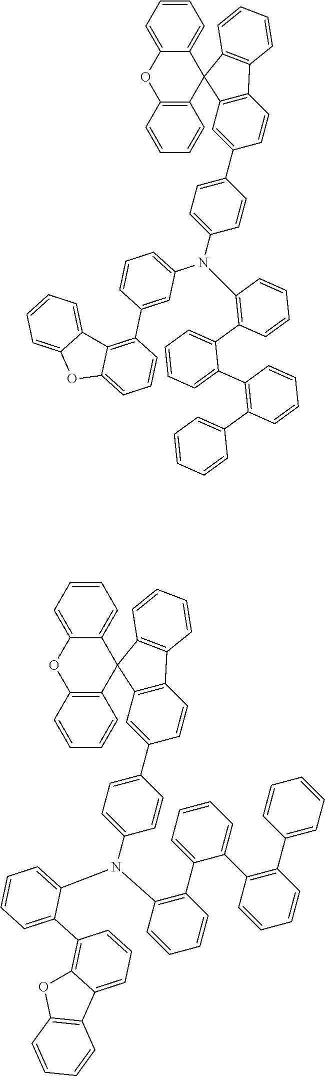

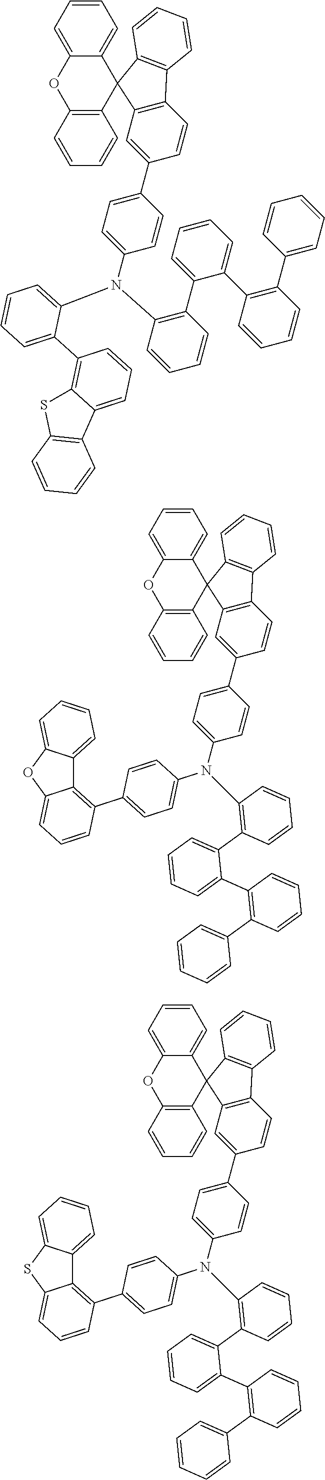

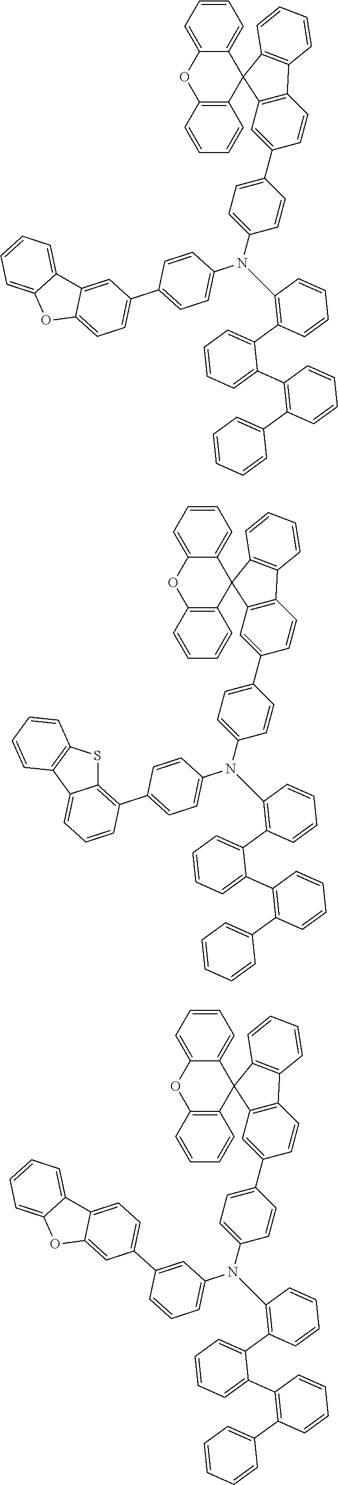

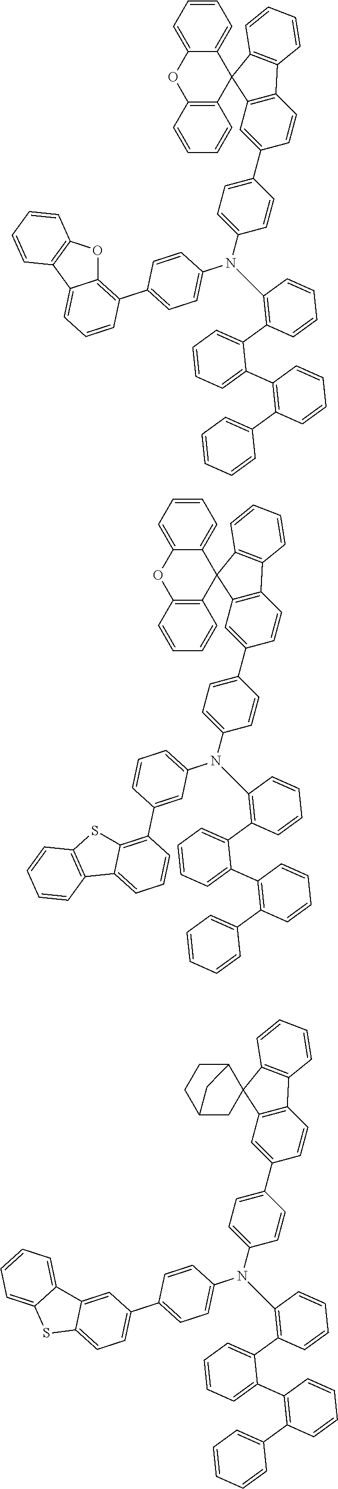

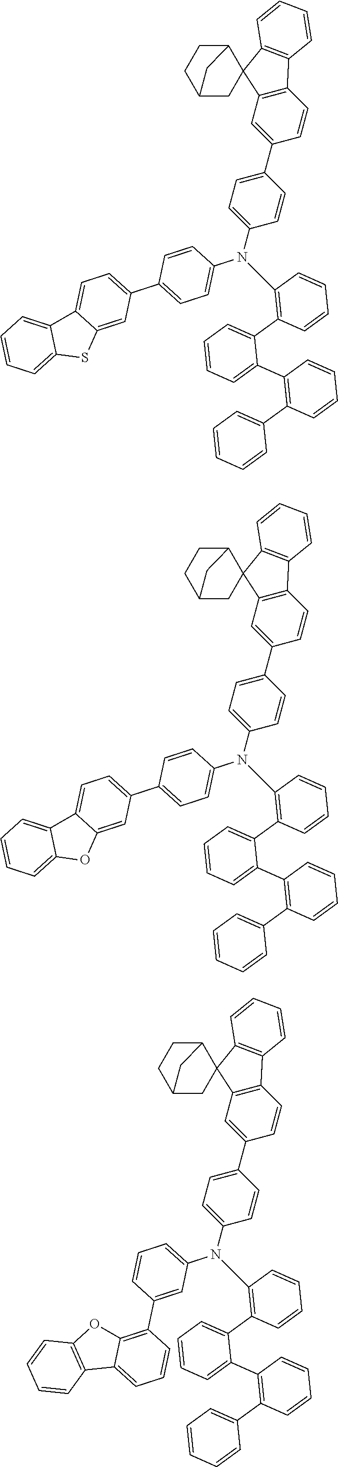

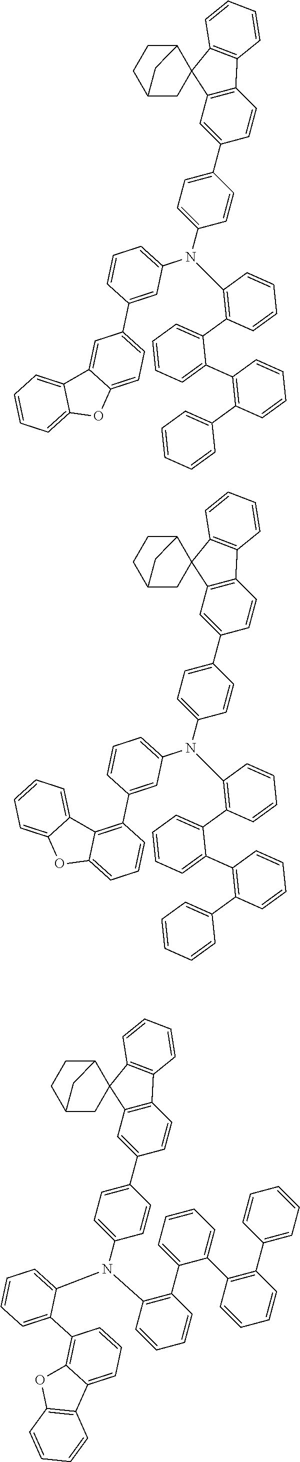 [SMILES: c1ccc(-c2ccccc2-c2ccccc2-c2ccccc2N(c2ccc(-c3ccc4c(c3)C3(CC5CCC3C5)c3ccccc3-4)cc2)c2cccc(-c3ccc4oc5ccccc5c4c3)c2)cc1 ; c1ccc(-c2ccccc2-c2ccccc2-c2ccccc2N(c2ccc(-c3ccc4c(c3)C3(CC5CCC3C5)c3ccccc3-4)cc2)c2cccc(-c3cccc4oc5ccccc5c34)c2)cc1 ; c1ccc(-c2ccccc2-c2ccccc2-c2ccccc2N(c2ccc(-c3ccc4c(c3)C3(CC5CCC3C5)c3ccccc3-4)cc2)c2ccccc2-c2cccc3c2oc2ccccc23)cc1]

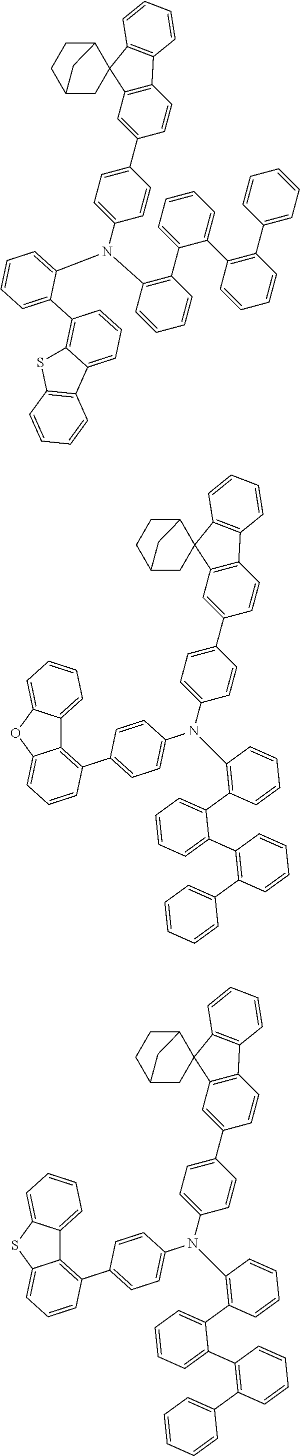 [SMILES: c1ccc(-c2ccccc2-c2ccccc2-c2ccccc2N(c2ccc(-c3ccc4c(c3)C3(CC5CCC3C5)c3ccccc3-4)cc2)c2ccccc2-c2cccc3c2sc2ccccc23)cc1 ; c1ccc(-c2ccccc2-c2ccccc2-c2ccccc2N(c2ccc(-c3ccc4c(c3)C3(CC5CCC3C5)c3ccccc3-4)cc2)c2ccc(-c3cccc4oc5ccccc5c34)cc2)cc1 ; c1ccc(-c2ccccc2-c2ccccc2-c2ccccc2N(c2ccc(-c3ccc4c(c3)C3(CC5CCC3C5)c3ccccc3-4)cc2)c2ccc(-c3cccc4sc5ccccc5c34)cc2)cc1]

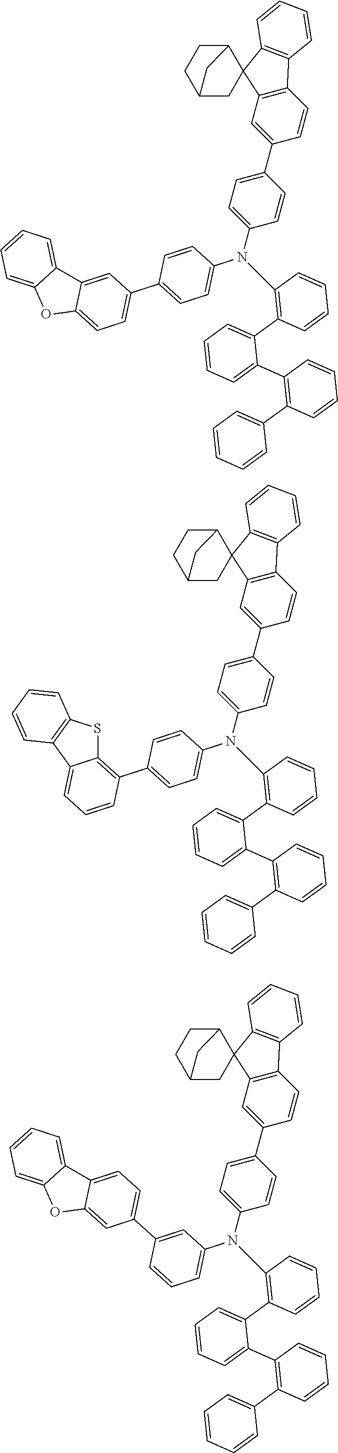

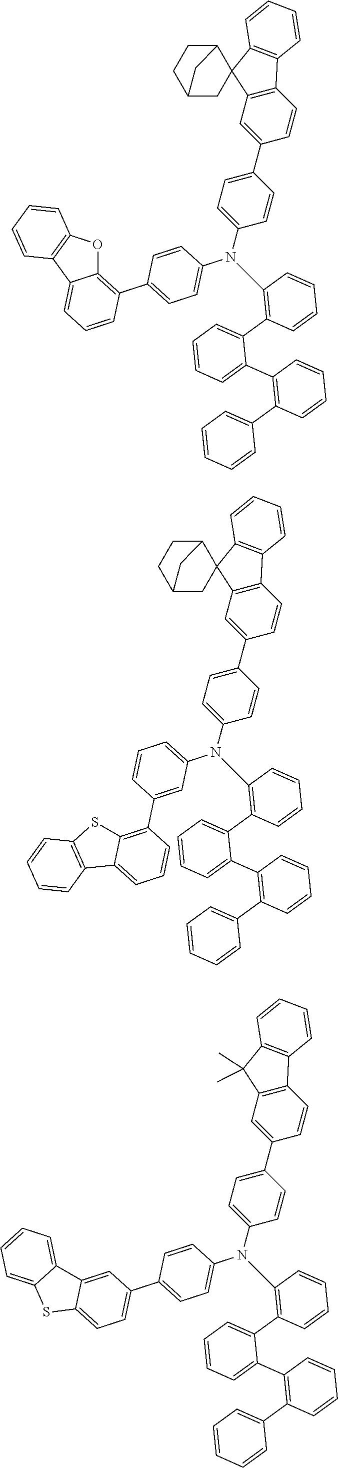

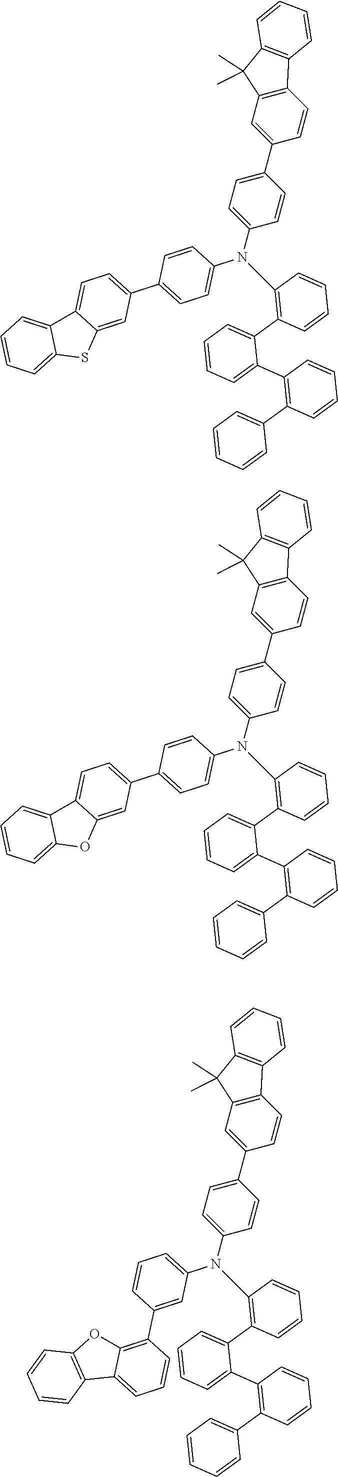

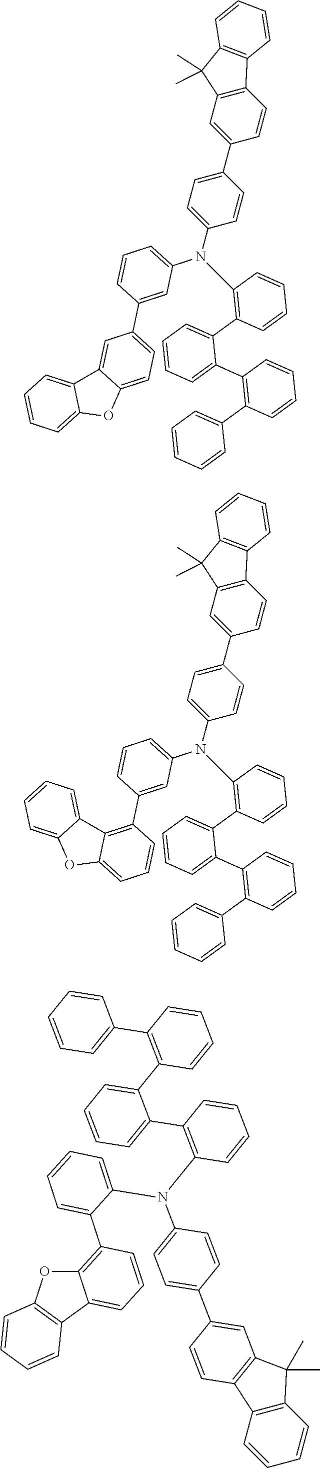 [SMILES: CC1(C)c2ccccc2-c2ccc(-c3ccc(N(c4cccc(-c5ccc6oc7ccccc7c6c5)c4)c4ccccc4-c4ccccc4-c4ccccc4-c4ccccc4)cc3)cc21 ; CC1(C)c2ccccc2-c2ccc(-c3ccc(N(c4cccc(-c5cccc6oc7ccccc7c56)c4)c4ccccc4-c4ccccc4-c4ccccc4-c4ccccc4)cc3)cc21 ; CC1(C)c2ccccc2-c2ccc(-c3ccc(N(c4ccccc4-c4ccccc4-c4ccccc4-c4ccccc4)c4ccccc4-c4cccc5c4oc4ccccc45)cc3)cc21]

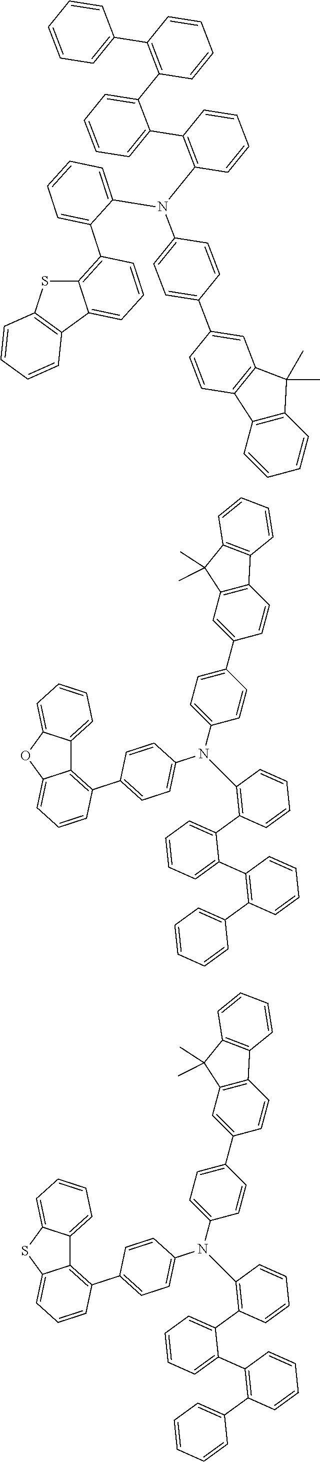

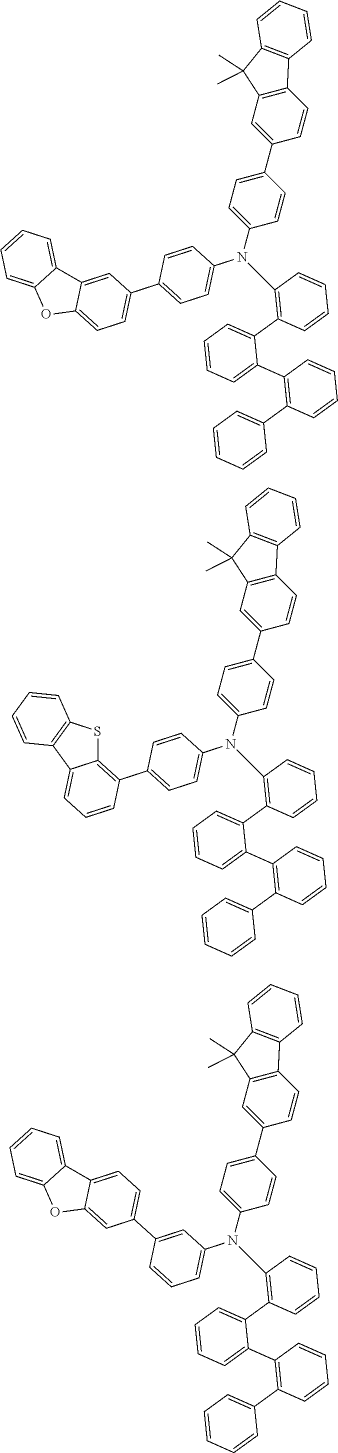

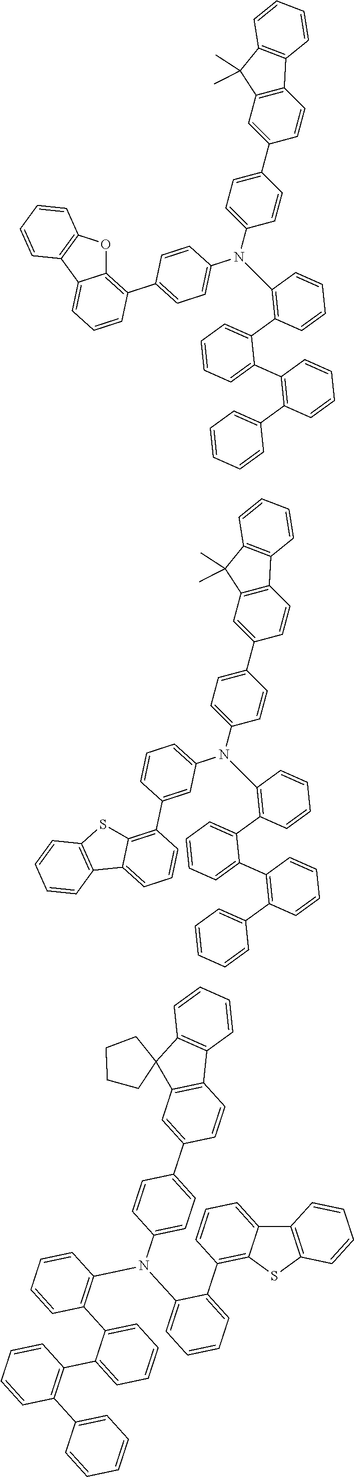

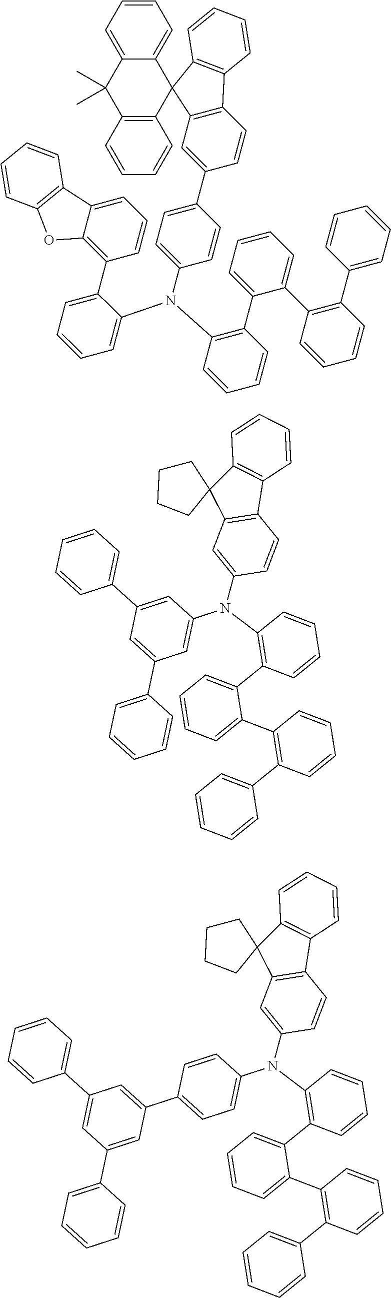

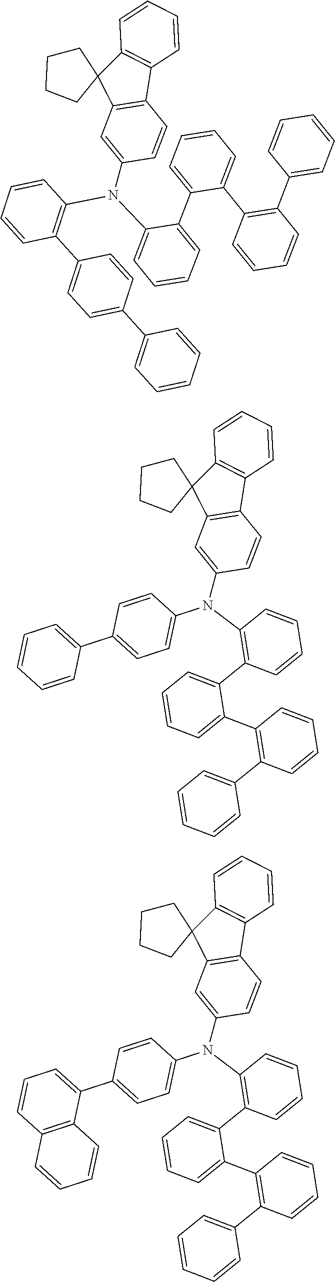

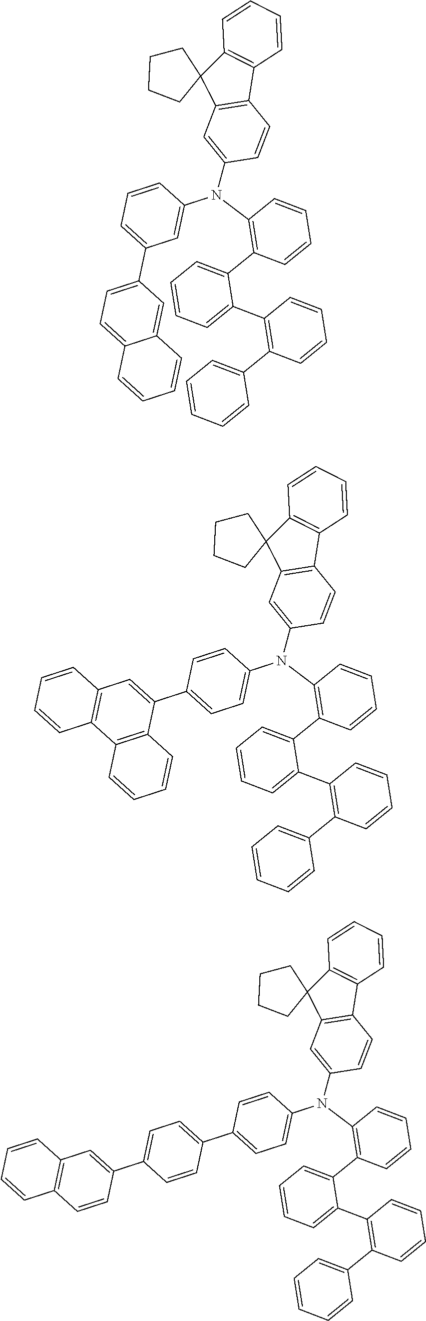 [SMILES: c1ccc(-c2ccccc2-c2ccccc2-c2ccccc2N(c2cccc(-c3ccc4ccccc4c3)c2)c2ccc3c(c2)C2(CCCC2)c2ccccc2-3)cc1 ; c1ccc(-c2ccccc2-c2ccccc2-c2ccccc2N(c2ccc(-c3cc4ccccc4c4ccccc34)cc2)c2ccc3c(c2)C2(CCCC2)c2ccccc2-3)cc1 ; c1ccc(-c2ccccc2-c2ccccc2-c2ccccc2N(c2ccc(-c3ccc(-c4ccc5ccccc5c4)cc3)cc2)c2ccc3c(c2)C2(CCCC2)c2ccccc2-3)cc1]

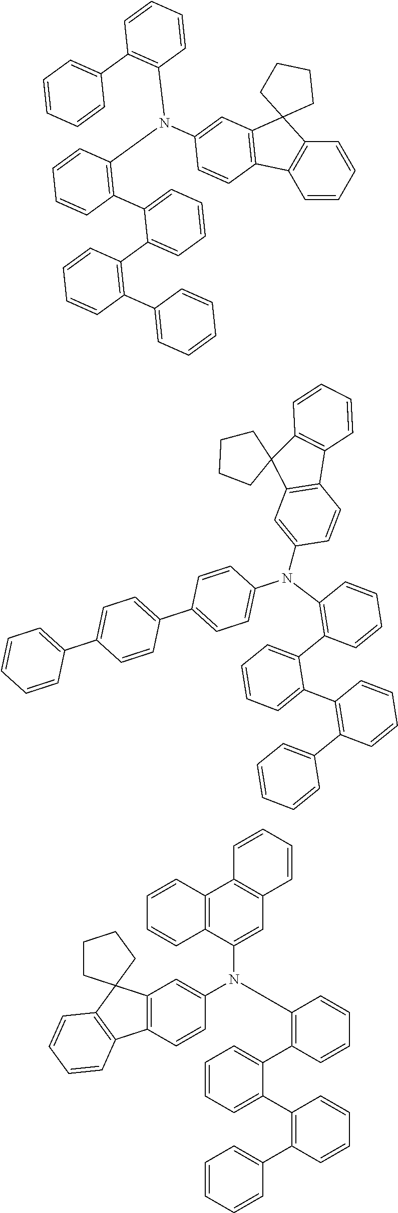

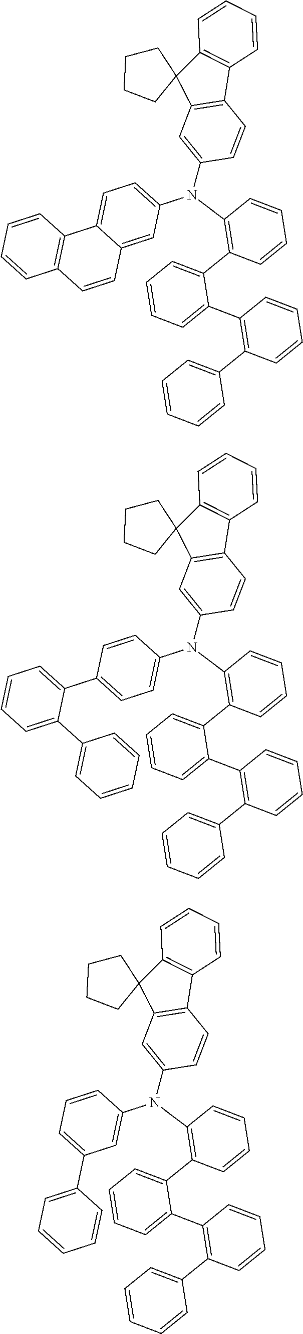

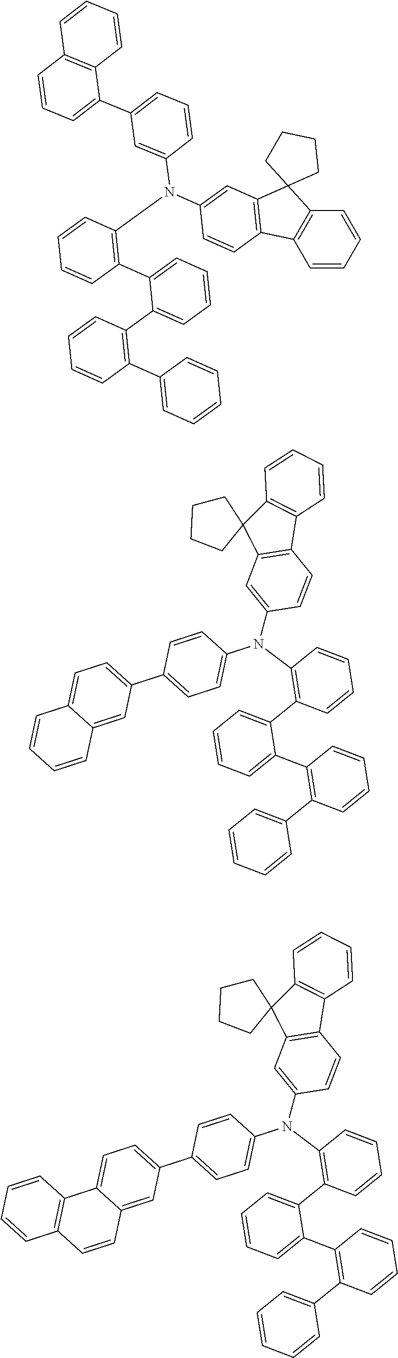 [SMILES: c1ccc(-c2ccccc2-c2ccccc2-c2ccccc2N(c2cccc(-c3cccc4ccccc34)c2)c2ccc3c(c2)C2(CCCC2)c2ccccc2-3)cc1 ; c1ccc(-c2ccccc2-c2ccccc2-c2ccccc2N(c2ccc(-c3ccc4ccccc4c3)cc2)c2ccc3c(c2)C2(CCCC2)c2ccccc2-3)cc1 ; c1ccc(-c2ccccc2-c2ccccc2-c2ccccc2N(c2ccc(-c3ccc4c(ccc5ccccc54)c3)cc2)c2ccc3c(c2)C2(CCCC2)c2ccccc2-3)cc1]

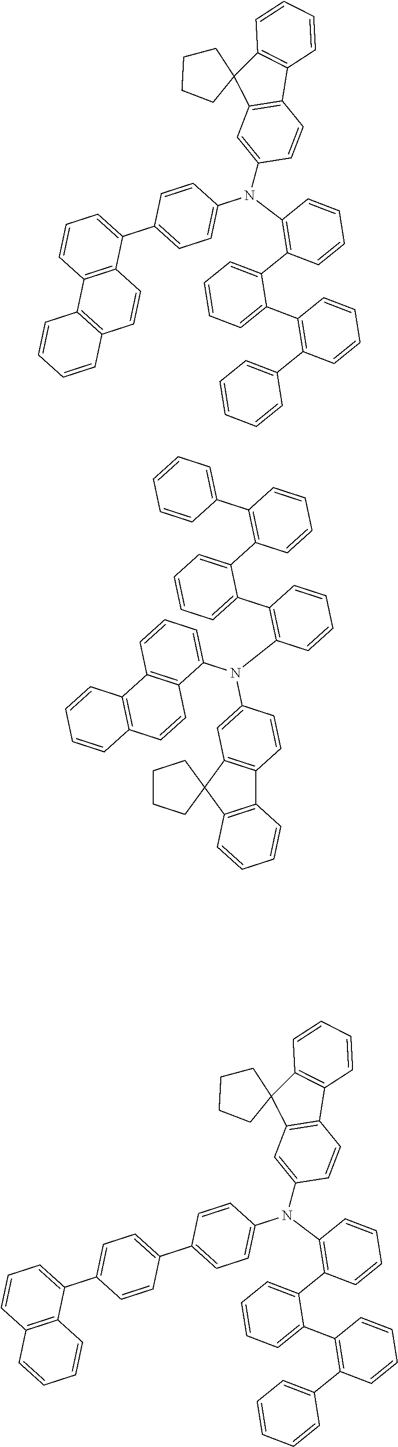 [SMILES: c1ccc(-c2ccccc2-c2ccccc2-c2ccccc2N(c2ccc(-c3cccc4c3ccc3ccccc34)cc2)c2ccc3c(c2)C2(CCCC2)c2ccccc2-3)cc1 ; c1ccc(-c2ccccc2-c2ccccc2-c2ccccc2N(c2ccc3c(c2)C2(CCCC2)c2ccccc2-3)c2cccc3c2ccc2ccccc23)cc1 ; c1ccc(-c2ccccc2-c2ccccc2-c2ccccc2N(c2ccc(-c3ccc(-c4cccc5ccccc45)cc3)cc2)c2ccc3c(c2)C2(CCCC2)c2ccccc2-3)cc1]

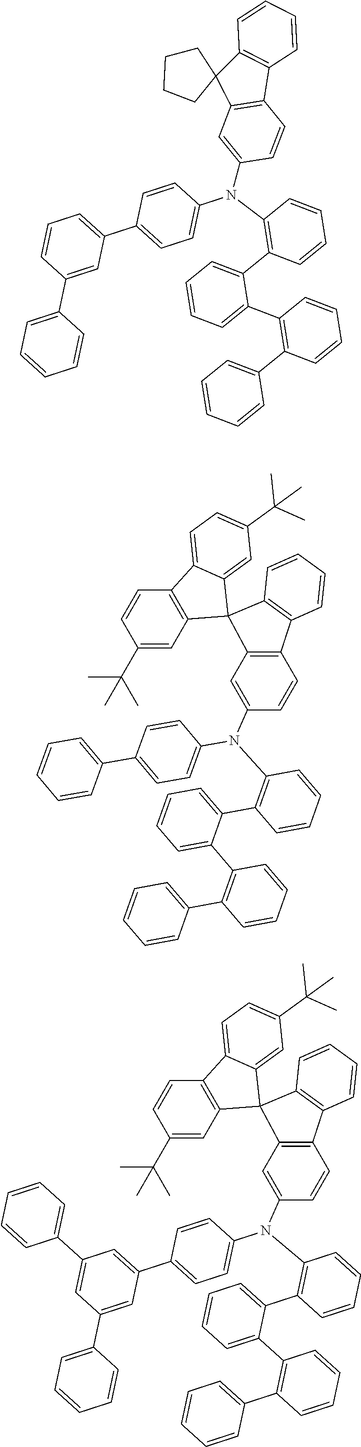 [SMILES: c1ccc(-c2cccc(-c3ccc(N(c4ccc5c(c4)C4(CCCC4)c4ccccc4-5)c4ccccc4-c4ccccc4-c4ccccc4-c4ccccc4)cc3)c2)cc1 ; CC(C)(C)c1ccc2c(c1)C1(c3ccccc3-c3ccc(N(c4ccc(-c5ccccc5)cc4)c4ccccc4-c4ccccc4-c4ccccc4-c4ccccc4)cc31)c1cc(C(C)(C)C)ccc1-2 ; CC(C)(C)c1ccc2c(c1)C1(c3ccccc3-c3ccc(N(c4ccc(-c5cc(-c6ccccc6)cc(-c6ccccc6)c5)cc4)c4ccccc4-c4ccccc4-c4ccccc4-c4ccccc4)cc31)c1cc(C(C)(C)C)ccc1-2]

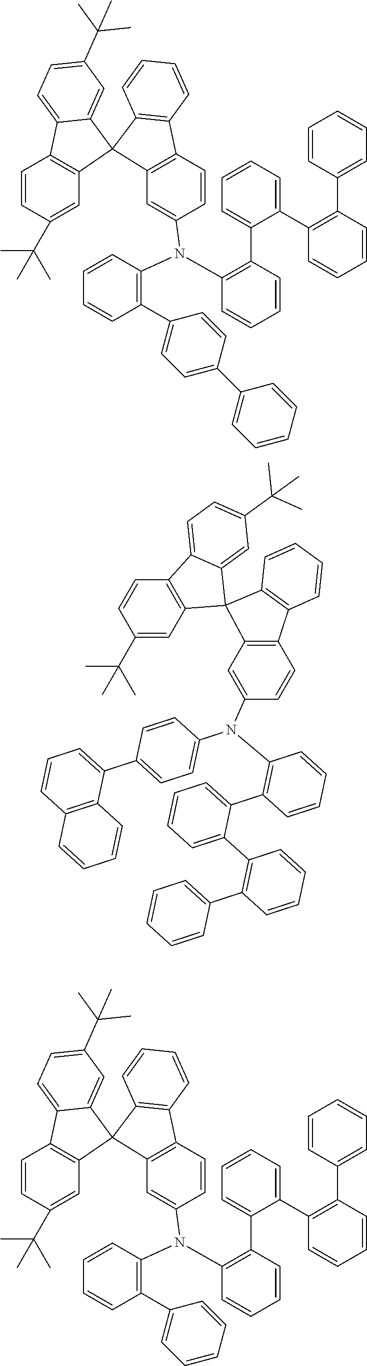

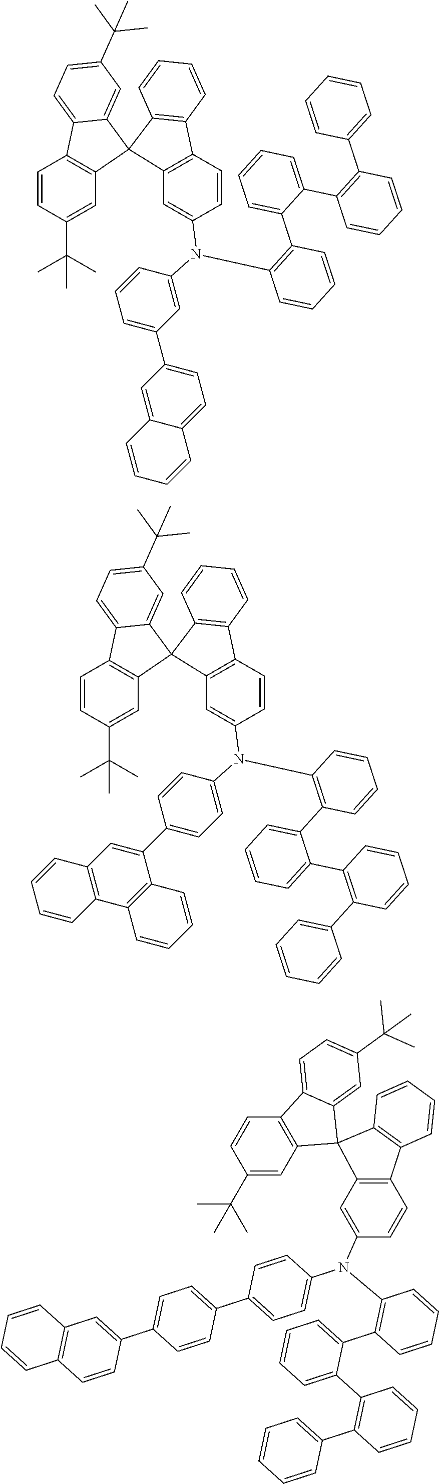

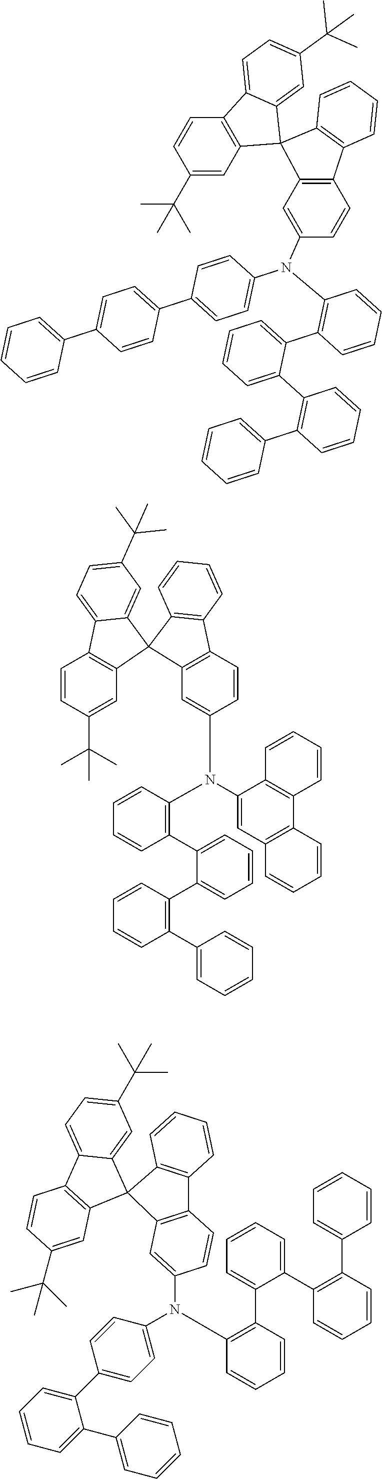 [SMILES: CC(C)(C)c1ccc2c(c1)C1(c3ccccc3-c3ccc(N(c4ccc(-c5ccc(-c6ccccc6)cc5)cc4)c4ccccc4-c4ccccc4-c4ccccc4-c4ccccc4)cc31)c1cc(C(C)(C)C)ccc1-2 ; CC(C)(C)c1ccc2c(c1)C1(c3ccccc3-c3ccc(CN(c4ccccc4-c4ccccc4-c4ccccc4-c4ccccc4)c4cc5ccccc5c5ccccc45)cc31)c1cc(C(C)(C)C)ccc1-2 ; CC(C)(C)c1ccc2c(c1)C1(c3ccccc3-c3ccc(N(c4ccc(-c5ccccc5-c5ccccc5)cc4)c4ccccc4-c4ccccc4-c4ccccc4-c4ccccc4)cc31)c1cc(C(C)(C)C)ccc1-2]

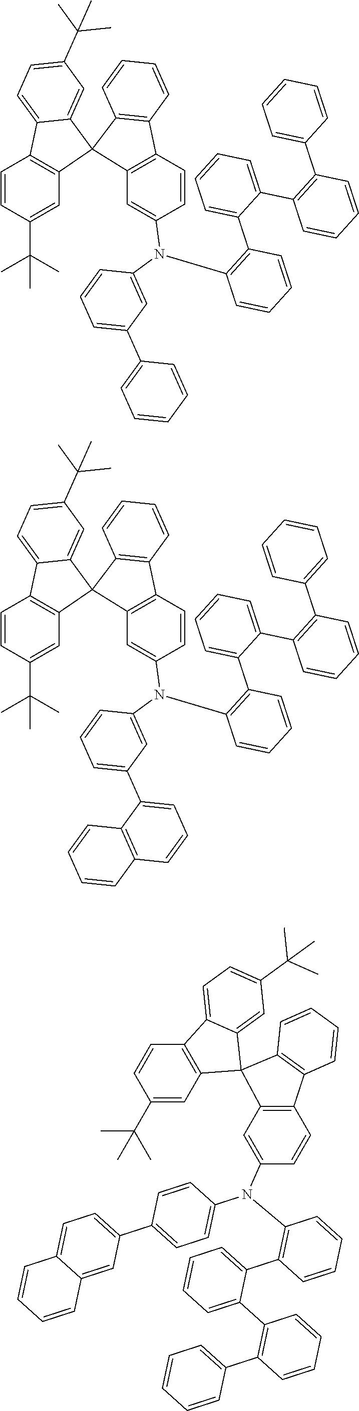 [SMILES: CC(C)(C)c1ccc2c(c1)C1(c3ccccc3-c3ccc(N(c4cccc(-c5ccccc5)c4)c4ccccc4-c4ccccc4-c4ccccc4-c4ccccc4)cc31)c1cc(C(C)(C)C)ccc1-2 ; CC(C)(C)c1ccc2c(c1)C1(c3ccccc3-c3ccc(N(c4cccc(-c5cccc6ccccc56)c4)c4ccccc4-c4ccccc4-c4ccccc4-c4ccccc4)cc31)c1cc(C(C)(C)C)ccc1-2 ; CC(C)(C)c1ccc2c(c1)C1(c3ccccc3-c3ccc(N(c4ccc(-c5ccc6ccccc6c5)cc4)c4ccccc4-c4ccccc4-c4ccccc4-c4ccccc4)cc31)c1cc(C(C)(C)C)ccc1-2]

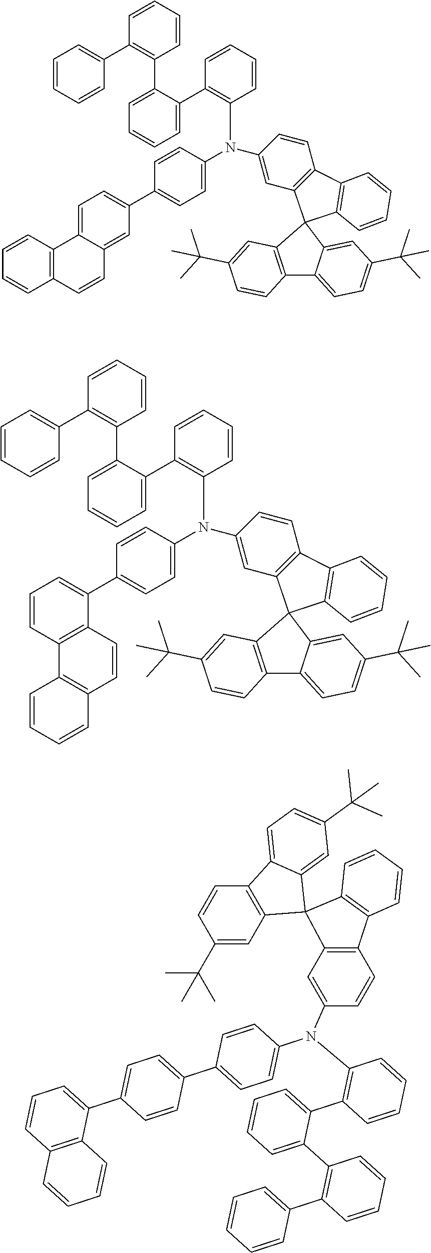 [SMILES: CC(C)(C)c1ccc2c(c1)C1(c3ccccc3-c3ccc(N(c4ccc(-c5ccc6c(ccc7ccccc76)c5)cc4)c4ccccc4-c4ccccc4-c4ccccc4-c4ccccc4)cc31)c1cc(C(C)(C)C)ccc1-2 ; CC(C)(C)c1ccc2c(c1)C1(c3ccccc3-c3ccc(N(c4ccc(-c5cccc6c5ccc5ccccc56)cc4)c4ccccc4-c4ccccc4-c4ccccc4-c4ccccc4)cc31)c1cc(C(C)(C)C)ccc1-2 ; CC(C)(C)c1ccc2c(c1)C1(c3ccccc3-c3ccc(N(c4ccc(-c5ccc(-c6cccc7ccccc67)cc5)cc4)c4ccccc4-c4ccccc4-c4ccccc4-c4ccccc4)cc31)c1cc(C(C)(C)C)ccc1-2]

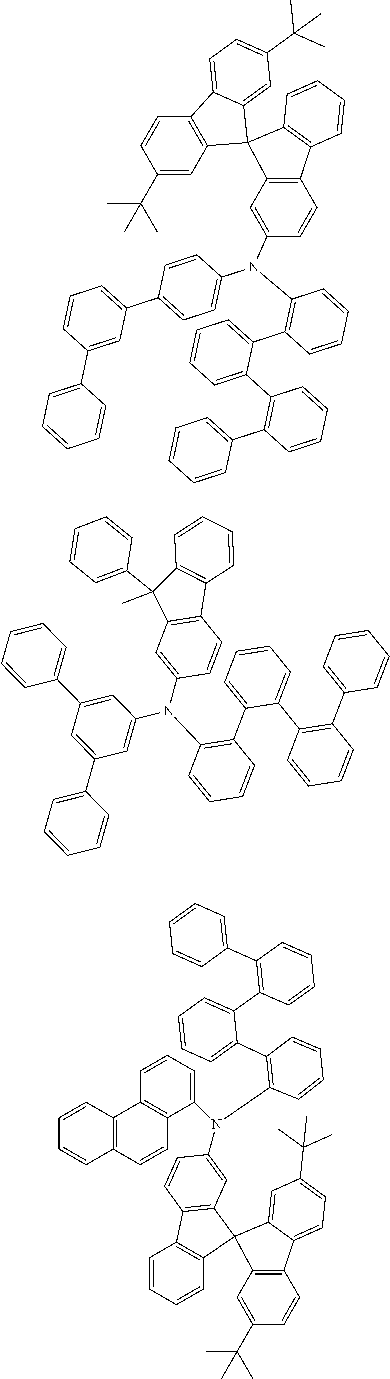

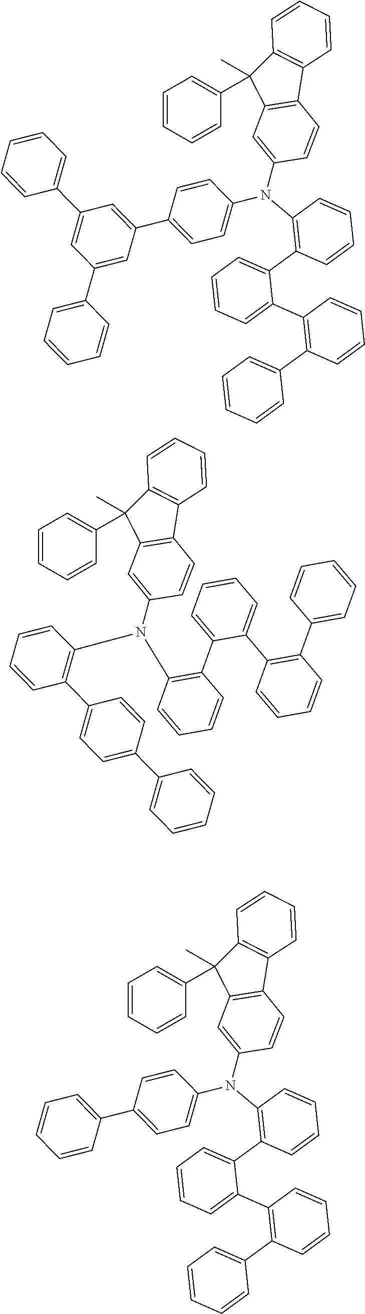 [SMILES: CC1(c2ccccc2)c2ccccc2-c2ccc(N(c3ccc(-c4cc(-c5ccccc5)cc(-c5ccccc5)c4)cc3)c3ccccc3-c3ccccc3-c3ccccc3-c3ccccc3)cc21 ; CC1(c2ccccc2)c2ccccc2-c2ccc(N(c3ccccc3-c3ccc(-c4ccccc4)cc3)c3ccccc3-c3ccccc3-c3ccccc3-c3ccccc3)cc21 ; CC1(c2ccccc2)c2ccccc2-c2ccc(N(c3ccc(-c4ccccc4)cc3)c3ccccc3-c3ccccc3-c3ccccc3-c3ccccc3)cc21]

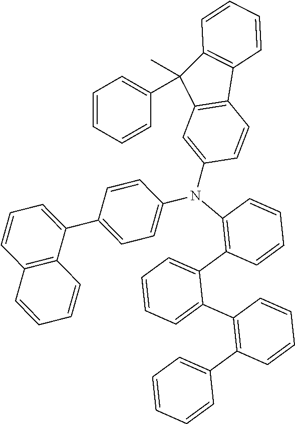 [SMILES: CC1(c2ccccc2)c2ccccc2-c2ccc(N(c3ccc(-c4cccc5ccccc45)cc3)c3ccccc3-c3ccccc3-c3ccccc3-c3ccccc3)cc21]

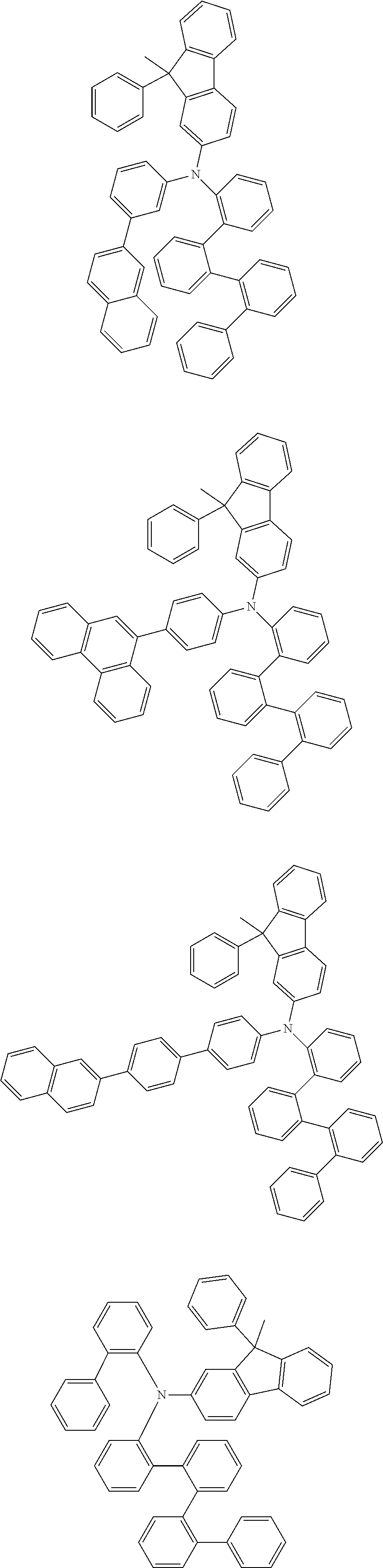 [SMILES: CC1(c2ccccc2)c2ccccc2-c2ccc(N(c3cccc(-c4ccc5ccccc5c4)c3)c3ccccc3-c3ccccc3-c3ccccc3-c3ccccc3)cc21 ; CC1(c2ccccc2)c2ccccc2-c2ccc(N(c3ccc(-c4cc5ccccc5c5ccccc45)cc3)c3ccccc3-c3ccccc3-c3ccccc3-c3ccccc3)cc21 ; CC1(c2ccccc2)c2ccccc2-c2ccc(N(c3ccc(-c4ccc(-c5ccc6ccccc6c5)cc4)cc3)c3ccccc3-c3ccccc3-c3ccccc3-c3ccccc3)cc21 ; CC1(c2ccccc2)c2ccccc2-c2ccc(N(c3ccccc3-c3ccccc3)c3ccccc3-c3ccccc3-c3ccccc3-c3ccccc3)cc21]

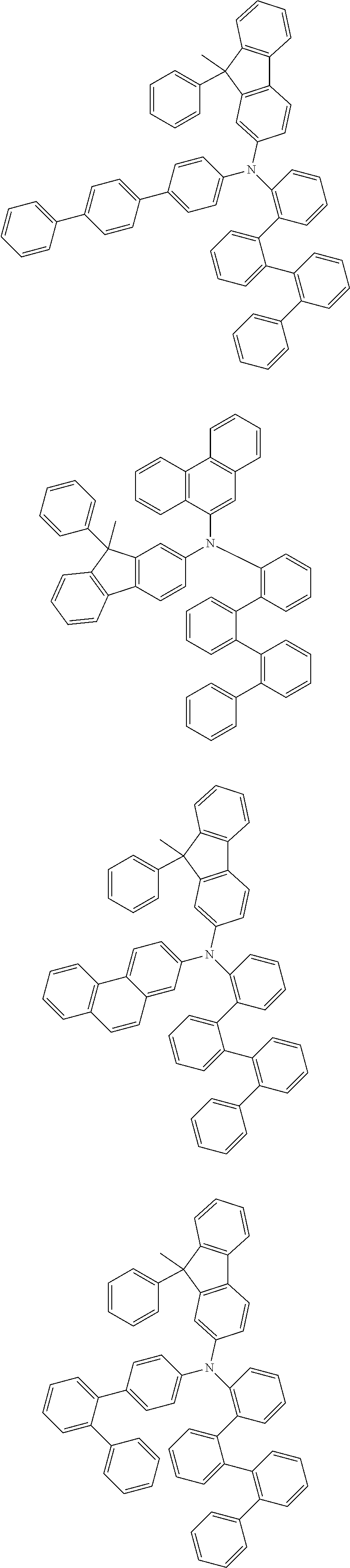 [SMILES: CC1(c2ccccc2)c2ccccc2-c2ccc(N(c3ccc(-c4ccc(-c5ccccc5)cc4)cc3)c3ccccc3-c3ccccc3-c3ccccc3-c3ccccc3)cc21 ; CC1(c2ccccc2)c2ccccc2-c2ccc(N(c3ccccc3-c3ccccc3-c3ccccc3-c3ccccc3)c3cc4ccccc4c4ccccc34)cc21 ; CC1(c2ccccc2)c2ccccc2-c2ccc(N(c3ccc4c(ccc5ccccc54)c3)c3ccccc3-c3ccccc3-c3ccccc3-c3ccccc3)cc21 ; CC1(c2ccccc2)c2ccccc2-c2ccc(N(c3ccc(-c4ccccc4-c4ccccc4)cc3)c3ccccc3-c3ccccc3-c3ccccc3-c3ccccc3)cc21]

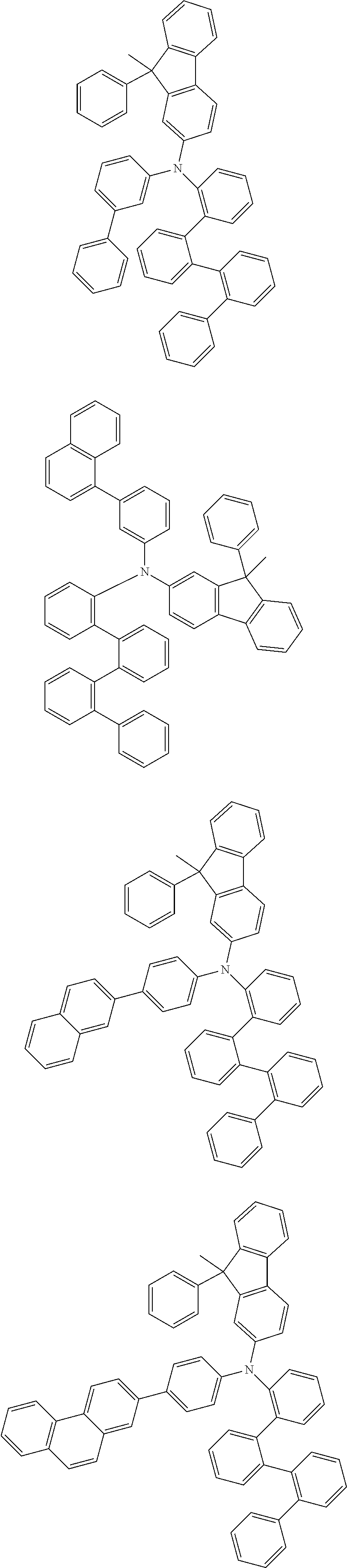

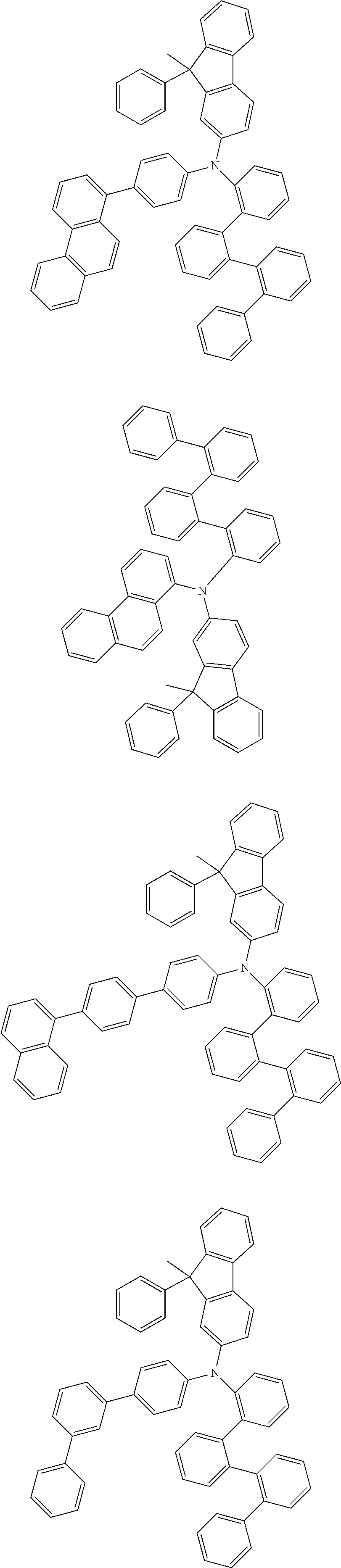 [SMILES: CC1(c2ccccc2)c2ccccc2-c2ccc(N(c3ccc(-c4cccc5c4ccc4ccccc45)cc3)c3ccccc3-c3ccccc3-c3ccccc3-c3ccccc3)cc21 ; CC1(c2ccccc2)c2ccccc2-c2ccc(N(c3ccccc3-c3ccccc3-c3ccccc3-c3ccccc3)c3cccc4c3ccc3ccccc34)cc21 ; CC1(c2ccccc2)c2ccccc2-c2ccc(N(c3ccc(-c4ccc(-c5cccc6ccccc56)cc4)cc3)c3ccccc3-c3ccccc3-c3ccccc3-c3ccccc3)cc21 ; CC1(c2ccccc2)c2ccccc2-c2ccc(N(c3ccc(-c4cccc(-c5ccccc5)c4)cc3)c3ccccc3-c3ccccc3-c3ccccc3-c3ccccc3)cc21]

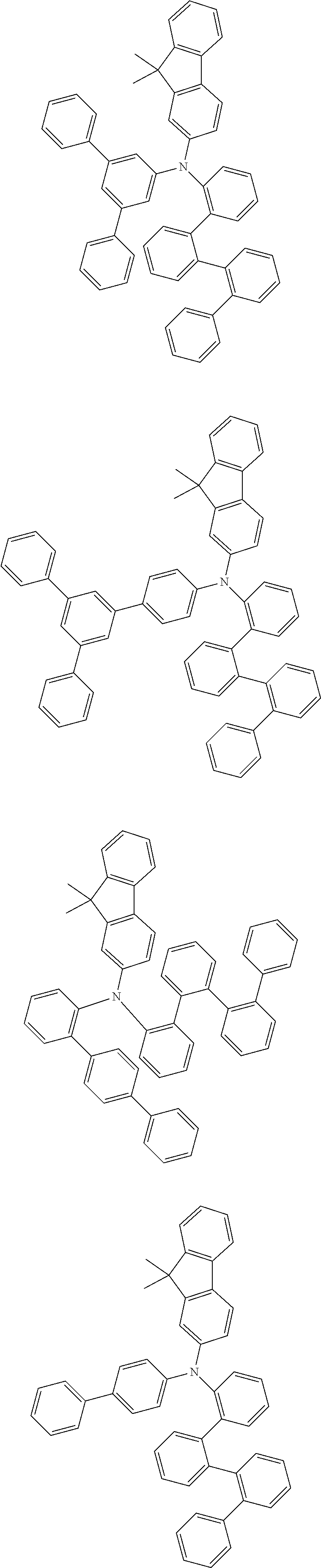 [SMILES: CC1(C)c2ccccc2-c2ccc(N(c3cc(-c4ccccc4)cc(-c4ccccc4)c3)c3ccccc3-c3ccccc3-c3ccccc3-c3ccccc3)cc21 ; CC1(C)c2ccccc2-c2ccc(N(c3ccc(-c4cc(-c5ccccc5)cc(-c5ccccc5)c4)cc3)c3ccccc3-c3ccccc3-c3ccccc3-c3ccccc3)cc21 ; CC1(C)c2ccccc2-c2ccc(N(c3ccccc3-c3ccc(-c4ccccc4)cc3)c3ccccc3-c3ccccc3-c3ccccc3-c3ccccc3)cc21 ; CC1(C)c2ccccc2-c2ccc(N(c3ccc(-c4ccccc4)cc3)c3ccccc3-c3ccccc3-c3ccccc3-c3ccccc3)cc21]

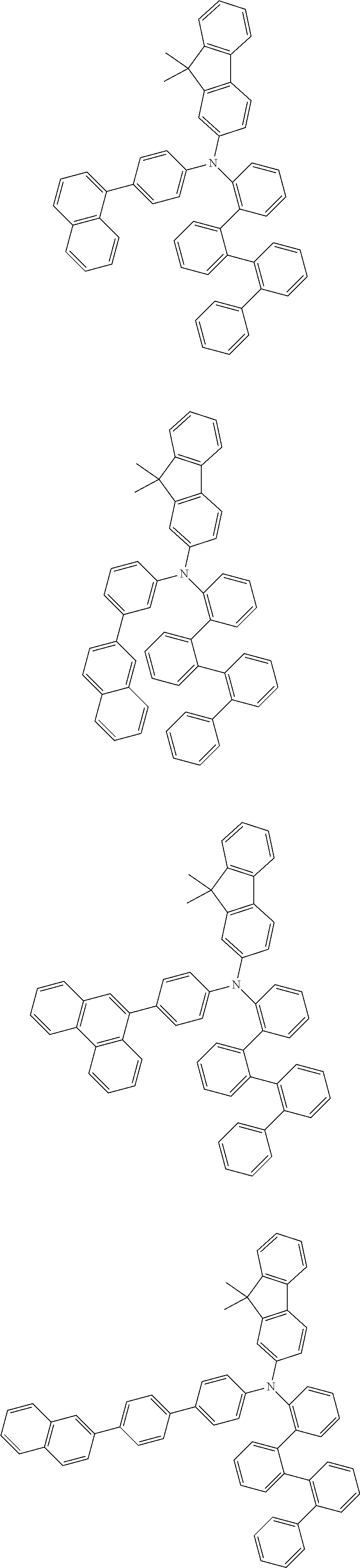 [SMILES: CC1(C)c2ccccc2-c2ccc(N(c3ccc(-c4cccc5ccccc45)cc3)c3ccccc3-c3ccccc3-c3ccccc3-c3ccccc3)cc21 ; CC1(C)c2ccccc2-c2ccc(N(c3cccc(-c4ccc5ccccc5c4)c3)c3ccccc3-c3ccccc3-c3ccccc3-c3ccccc3)cc21 ; CC1(C)c2ccccc2-c2ccc(N(c3ccc(-c4cc5ccccc5c5ccccc45)cc3)c3ccccc3-c3ccccc3-c3ccccc3-c3ccccc3)cc21 ; CC1(C)c2ccccc2-c2ccc(N(c3ccc(-c4ccc(-c5ccc6ccccc6c5)cc4)cc3)c3ccccc3-c3ccccc3-c3ccccc3-c3ccccc3)cc21]

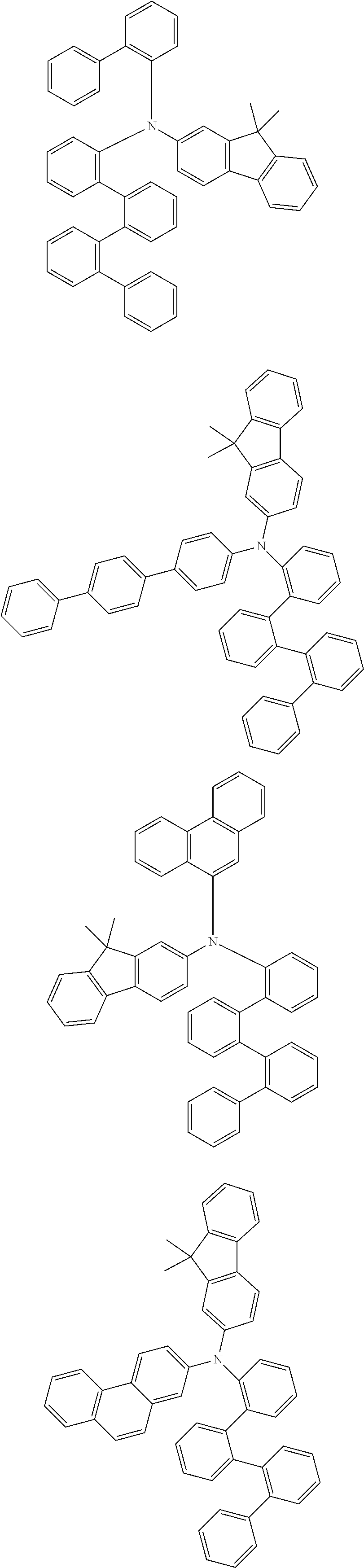

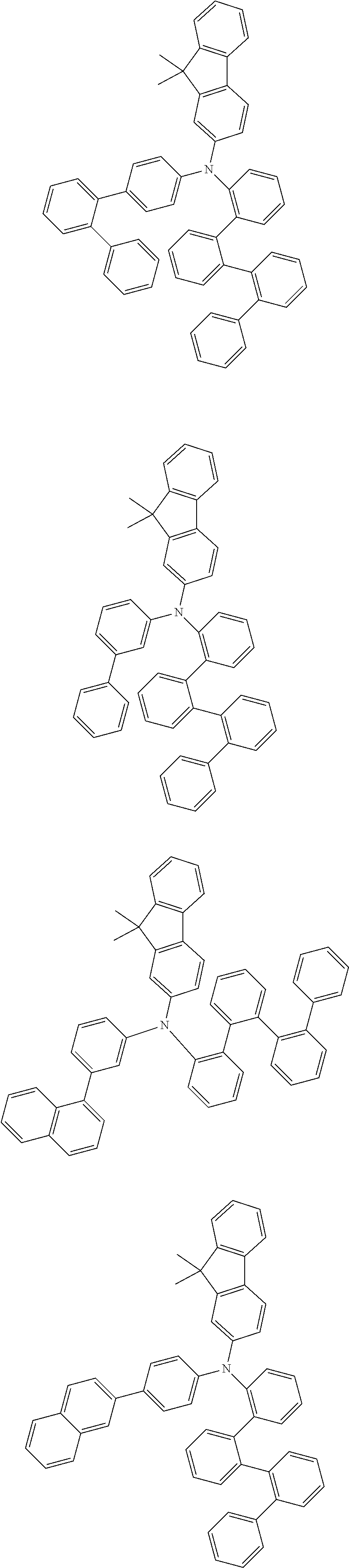 [SMILES: CC1(C)c2ccccc2-c2ccc(N(c3ccc(-c4ccccc4-c4ccccc4)cc3)c3ccccc3-c3ccccc3-c3ccccc3-c3ccccc3)cc21 ; CC1(C)c2ccccc2-c2ccc(N(c3cccc(-c4ccccc4)c3)c3ccccc3-c3ccccc3-c3ccccc3-c3ccccc3)cc21 ; CC1(C)c2ccccc2-c2ccc(N(c3cccc(-c4cccc5ccccc45)c3)c3ccccc3-c3ccccc3-c3ccccc3-c3ccccc3)cc21 ; CC1(C)c2ccccc2-c2ccc(N(c3ccc(-c4ccc5ccccc5c4)cc3)c3ccccc3-c3ccccc3-c3ccccc3-c3ccccc3)cc21]

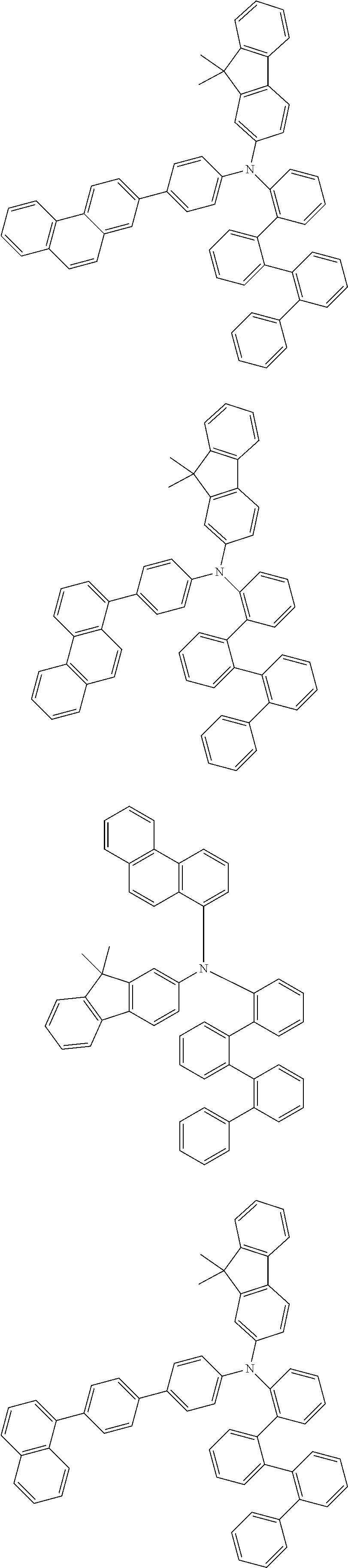 [SMILES: CC1(C)c2ccccc2-c2ccc(N(c3ccc(-c4ccc5c(ccc6ccccc65)c4)cc3)c3ccccc3-c3ccccc3-c3ccccc3-c3ccccc3)cc21 ; CC1(C)c2ccccc2-c2ccc(N(c3ccc(-c4cccc5c4ccc4ccccc45)cc3)c3ccccc3-c3ccccc3-c3ccccc3-c3ccccc3)cc21 ; CC1(C)c2ccccc2-c2ccc(N(c3ccccc3-c3ccccc3-c3ccccc3-c3ccccc3)c3cccc4c3ccc3ccccc34)cc21 ; CC1(C)c2ccccc2-c2ccc(N(c3ccc(-c4ccc(-c5cccc6ccccc56)cc4)cc3)c3ccccc3-c3ccccc3-c3ccccc3-c3ccccc3)cc21]

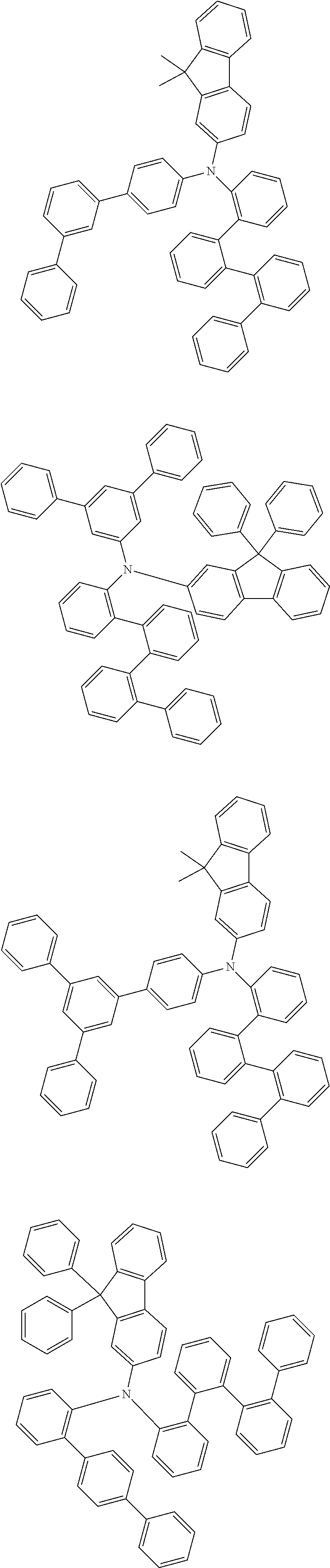 [SMILES: CC1(C)c2ccccc2-c2ccc(N(c3ccc(-c4cccc(-c5ccccc5)c4)cc3)c3ccccc3-c3ccccc3-c3ccccc3-c3ccccc3)cc21 ; c1ccc(-c2cc(-c3ccccc3)cc(N(c3ccc4c(c3)C(c3ccccc3)(c3ccccc3)c3ccccc3-4)c3ccccc3-c3ccccc3-c3ccccc3-c3ccccc3)c2)cc1 ; CC1(C)c2ccccc2-c2ccc(N(c3ccc(-c4cc(-c5ccccc5)cc(-c5ccccc5)c4)cc3)c3ccccc3-c3ccccc3-c3ccccc3-c3ccccc3)cc21 ; c1ccc(-c2ccc(-c3ccccc3N(c3ccc4c(c3)C(c3ccccc3)(c3ccccc3)c3ccccc3-4)c3ccccc3-c3ccccc3-c3ccccc3-c3ccccc3)cc2)cc1]

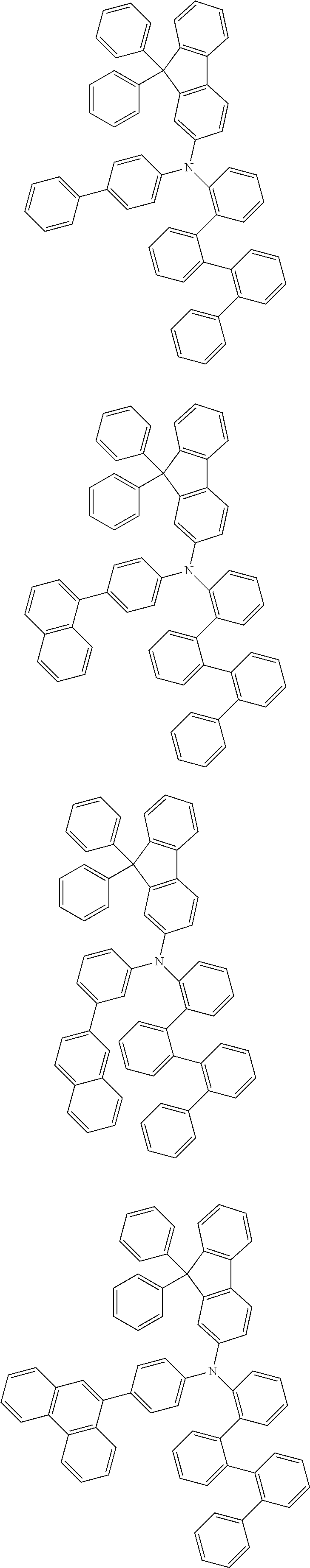

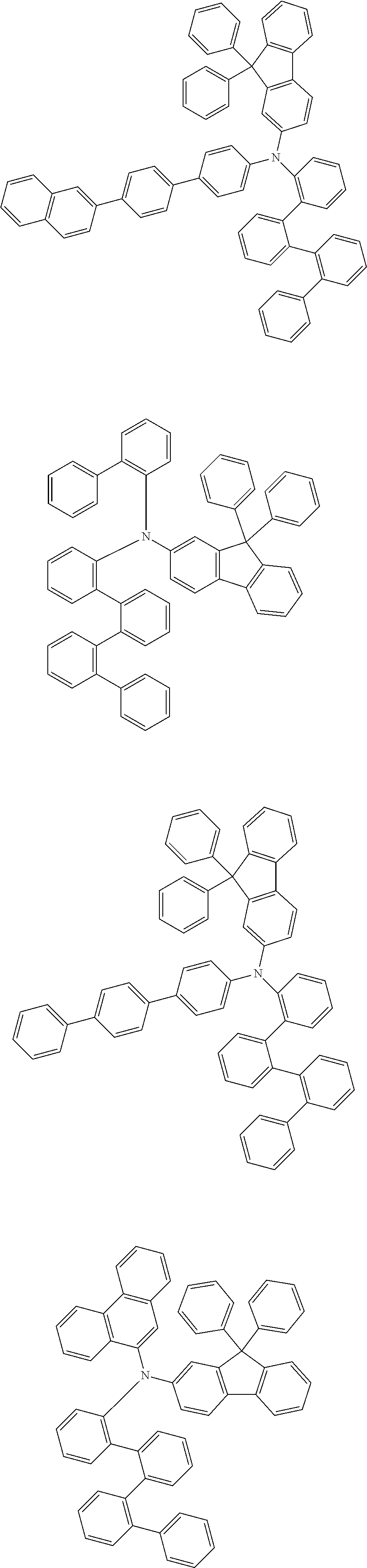 [SMILES: c1ccc(-c2ccccc2-c2ccccc2-c2ccccc2N(c2ccc(-c3ccc(-c4ccc5ccccc5c4)cc3)cc2)c2ccc3c(c2)C(c2ccccc2)(c2ccccc2)c2ccccc2-3)cc1 ; c1ccc(-c2ccccc2-c2ccccc2-c2ccccc2N(c2ccc3c(c2)C(c2ccccc2)(c2ccccc2)c2ccccc2-3)c2ccccc2-c2ccccc2)cc1 ; c1ccc(-c2ccc(-c3ccc(N(c4ccc5c(c4)C(c4ccccc4)(c4ccccc4)c4ccccc4-5)c4ccccc4-c4ccccc4-c4ccccc4-c4ccccc4)cc3)cc2)cc1 ; c1ccc(-c2ccccc2-c2ccccc2-c2ccccc2N(c2ccc3c(c2)C(c2ccccc2)(c2ccccc2)c2ccccc2-3)c2cc3ccccc3c3ccccc23)cc1]

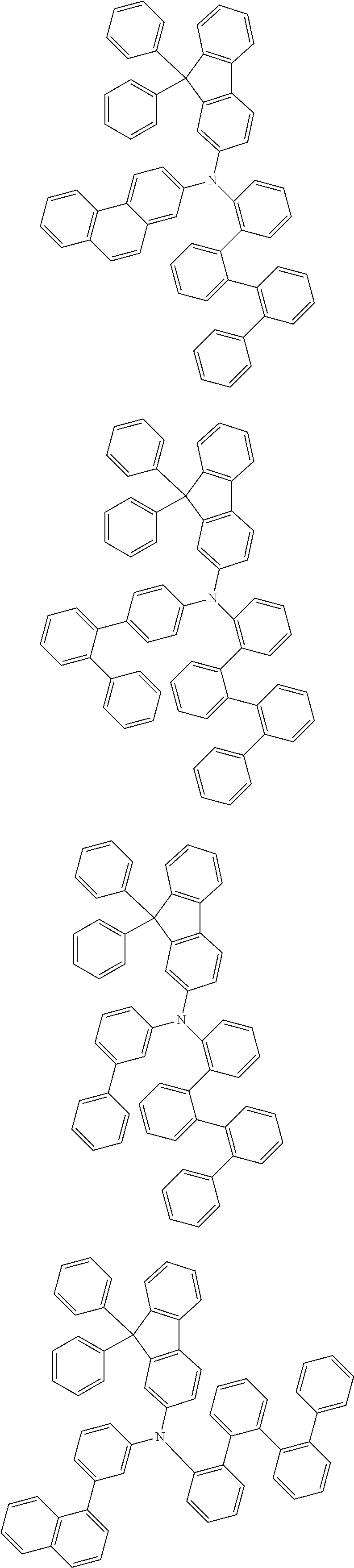

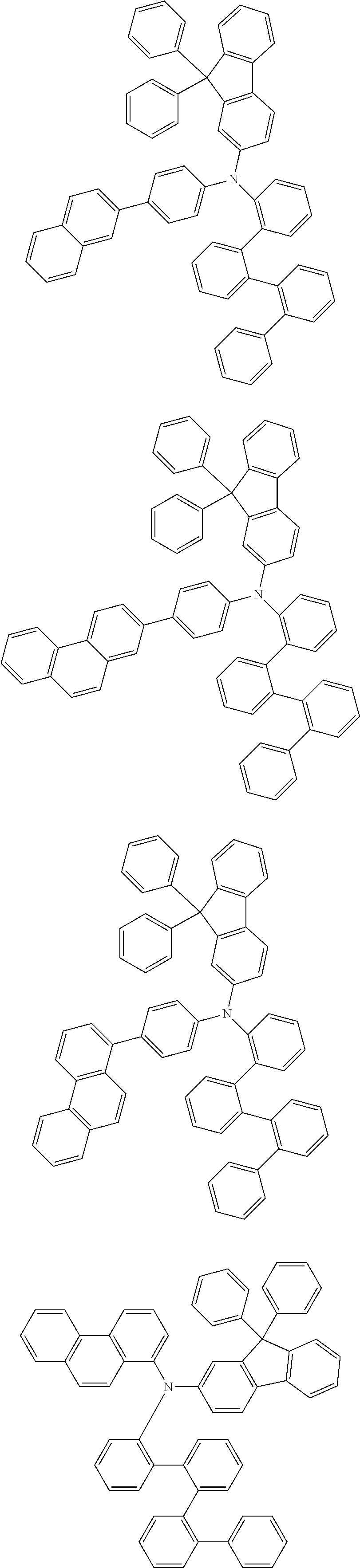

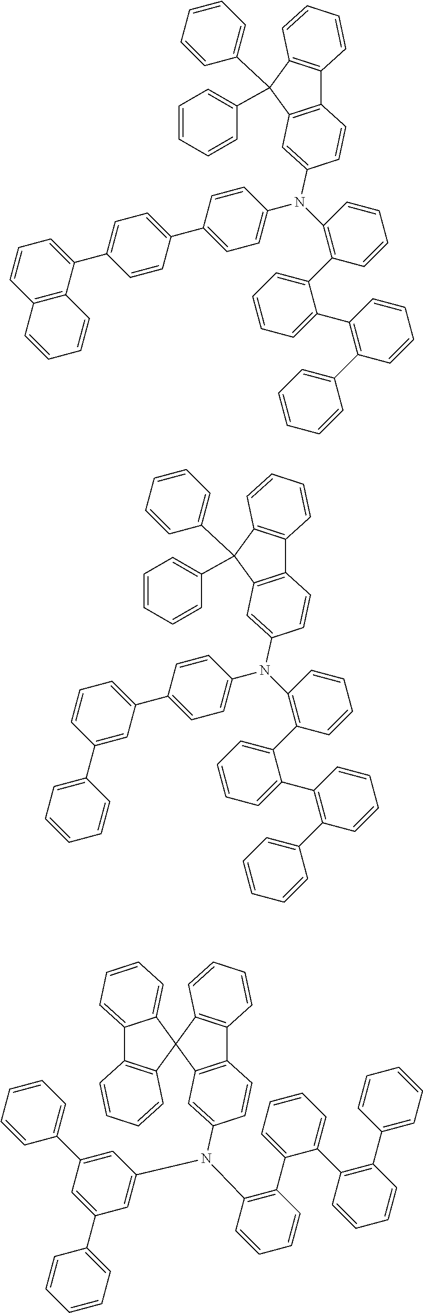 [SMILES: c1ccc(-c2ccccc2-c2ccccc2-c2ccccc2N(c2ccc(-c3ccc(-c4cccc5ccccc45)cc3)cc2)c2ccc3c(c2)C(c2ccccc2)(c2ccccc2)c2ccccc2-3)cc1 ; c1ccc(-c2cccc(-c3ccc(N(c4ccc5c(c4)C(c4ccccc4)(c4ccccc4)c4ccccc4-5)c4ccccc4-c4ccccc4-c4ccccc4-c4ccccc4)cc3)c2)cc1 ; c1ccc(-c2cc(-c3ccccc3)cc(N(c3ccc4c(c3)C3(c5ccccc5-c5ccccc53)c3ccccc3-4)c3ccccc3-c3ccccc3-c3ccccc3-c3ccccc3)c2)cc1]

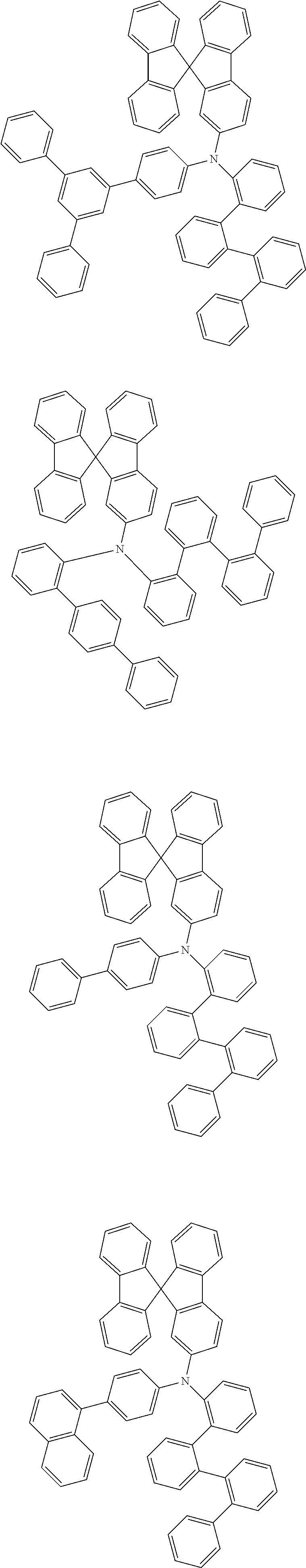 [SMILES: c1ccc(-c2cc(-c3ccccc3)cc(-c3ccc(N(c4ccc5c(c4)C4(c6ccccc6-c6ccccc64)c4ccccc4-5)c4ccccc4-c4ccccc4-c4ccccc4-c4ccccc4)cc3)c2)cc1 ; c1ccc(-c2ccc(-c3ccccc3N(c3ccc4c(c3)C3(c5ccccc5-c5ccccc53)c3ccccc3-4)c3ccccc3-c3ccccc3-c3ccccc3-c3ccccc3)cc2)cc1 ; c1ccc(-c2ccc(N(c3ccc4c(c3)C3(c5ccccc5-c5ccccc53)c3ccccc3-4)c3ccccc3-c3ccccc3-c3ccccc3-c3ccccc3)cc2)cc1 ; c1ccc(-c2ccccc2-c2ccccc2-c2ccccc2N(c2ccc(-c3cccc4ccccc34)cc2)c2ccc3c(c2)C2(c4ccccc4-c4ccccc42)c2ccccc2-3)cc1]

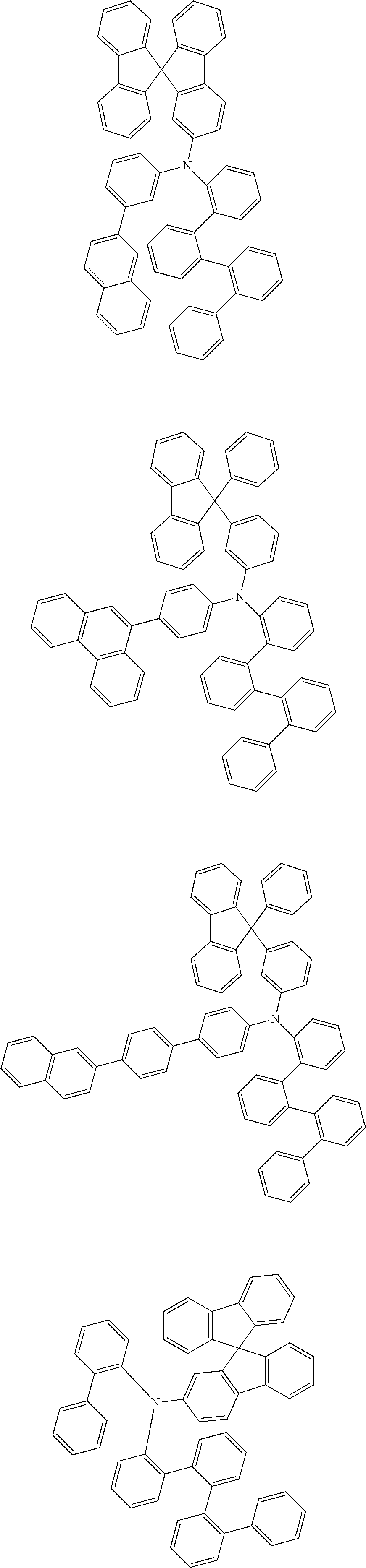 [SMILES: c1ccc(-c2ccccc2-c2ccccc2-c2ccccc2N(c2cccc(-c3ccc4ccccc4c3)c2)c2ccc3c(c2)C2(c4ccccc4-c4ccccc42)c2ccccc2-3)cc1 ; c1ccc(-c2ccccc2-c2ccccc2-c2ccccc2N(c2ccc(-c3cc4ccccc4c4ccccc34)cc2)c2ccc3c(c2)C2(c4ccccc4-c4ccccc42)c2ccccc2-3)cc1 ; c1ccc(-c2ccccc2-c2ccccc2-c2ccccc2N(c2ccc(-c3ccc(-c4ccc5ccccc5c4)cc3)cc2)c2ccc3c(c2)C2(c4ccccc4-c4ccccc42)c2ccccc2-3)cc1 ; c1ccc(-c2ccccc2-c2ccccc2-c2ccccc2N(c2ccc3c(c2)C2(c4ccccc4-c4ccccc42)c2ccccc2-3)c2ccccc2-c2ccccc2)cc1]

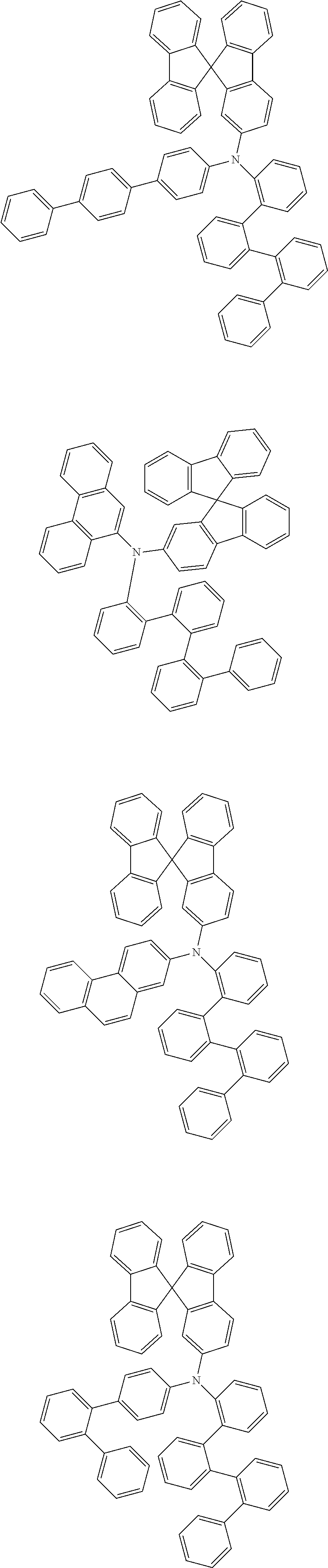 [SMILES: c1ccc(-c2ccc(-c3ccc(N(c4ccc5c(c4)C4(c6ccccc6-c6ccccc64)c4ccccc4-5)c4ccccc4-c4ccccc4-c4ccccc4-c4ccccc4)cc3)cc2)cc1 ; c1ccc(-c2ccccc2-c2ccccc2-c2ccccc2N(c2ccc3c(c2)C2(c4ccccc4-c4ccccc42)c2ccccc2-3)c2cc3ccccc3c3ccccc23)cc1 ; c1ccc(-c2ccccc2-c2ccccc2-c2ccccc2N(c2ccc3c(c2)C2(c4ccccc4-c4ccccc42)c2ccccc2-3)c2ccc3c(ccc4ccccc43)c2)cc1 ; c1ccc(-c2ccccc2-c2ccc(N(c3ccc4c(c3)C3(c5ccccc5-c5ccccc53)c3ccccc3-4)c3ccccc3-c3ccccc3-c3ccccc3-c3ccccc3)cc2)cc1]

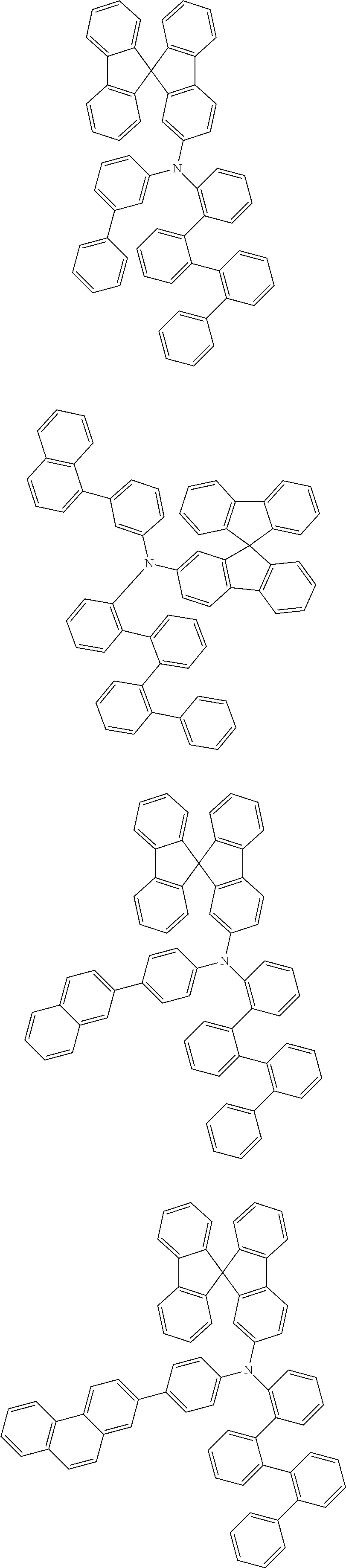 [SMILES: c1ccc(-c2cccc(N(c3ccc4c(c3)C3(c5ccccc5-c5ccccc53)c3ccccc3-4)c3ccccc3-c3ccccc3-c3ccccc3-c3ccccc3)c2)cc1 ; c1ccc(-c2ccccc2-c2ccccc2-c2ccccc2N(c2cccc(-c3cccc4ccccc34)c2)c2ccc3c(c2)C2(c4ccccc4-c4ccccc42)c2ccccc2-3)cc1 ; c1ccc(-c2ccccc2-c2ccccc2-c2ccccc2N(c2ccc(-c3ccc4ccccc4c3)cc2)c2ccc3c(c2)C2(c4ccccc4-c4ccccc42)c2ccccc2-3)cc1 ; c1ccc(-c2ccccc2-c2ccccc2-c2ccccc2N(c2ccc(-c3ccc4c(ccc5ccccc54)c3)cc2)c2ccc3c(c2)C2(c4ccccc4-c4ccccc42)c2ccccc2-3)cc1]

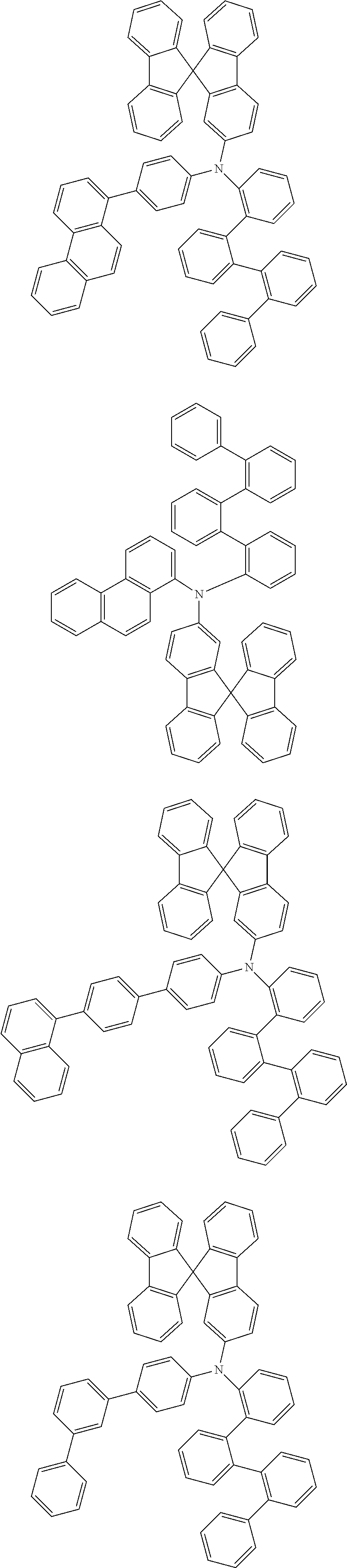

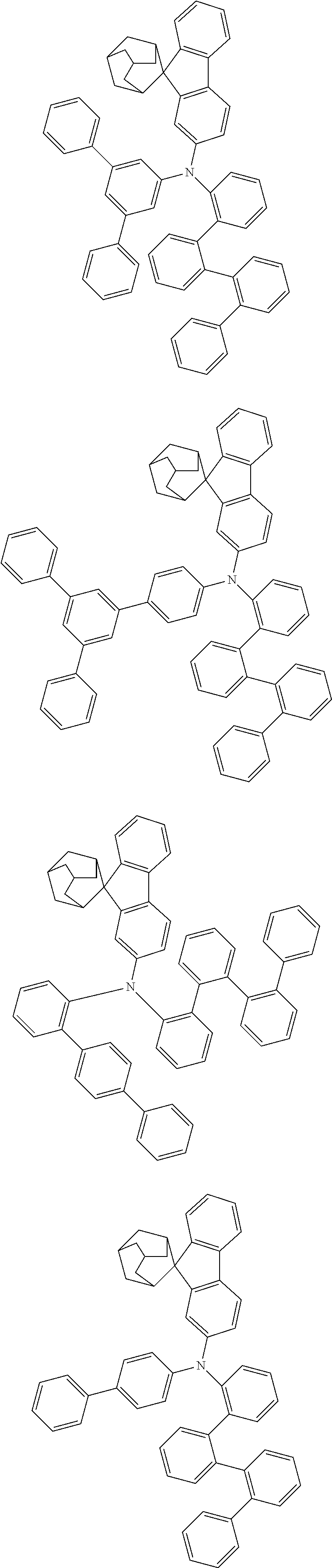 [SMILES: c1ccc(-c2cc(-c3ccccc3)cc(N(c3ccc4c(c3)C3(c5ccccc5-4)C4CC5CC(C4)CC3C5)c3ccccc3-c3ccccc3-c3ccccc3-c3ccccc3)c2)cc1 ; c1ccc(-c2cc(-c3ccccc3)cc(-c3ccc(N(c4ccc5c(c4)C4(c6ccccc6-5)C5CC6CC(C5)CC4C6)c4ccccc4-c4ccccc4-c4ccccc4-c4ccccc4)cc3)c2)cc1 ; c1ccc(-c2ccc(-c3ccccc3N(c3ccc4c(c3)C3(c5ccccc5-4)C4CC5CC(C4)CC3C5)c3ccccc3-c3ccccc3-c3ccccc3-c3ccccc3)cc2)cc1 ; c1ccc(-c2ccc(N(c3ccc4c(c3)C3(c5ccccc5-4)C4CC5CC(C4)CC3C5)c3ccccc3-c3ccccc3-c3ccccc3-c3ccccc3)cc2)cc1]

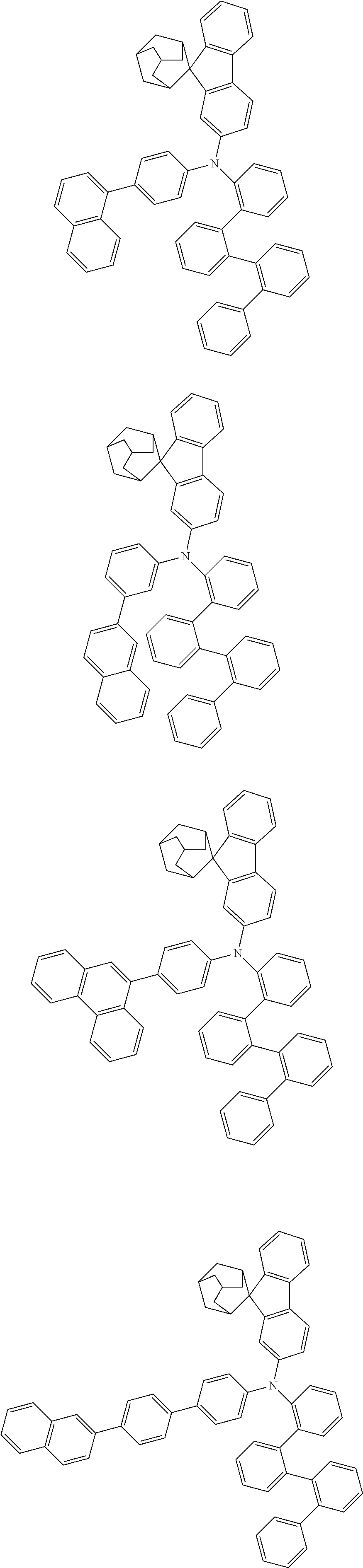 [SMILES: c1ccc(-c2ccccc2-c2ccccc2-c2ccccc2N(c2ccc(-c3cccc4ccccc34)cc2)c2ccc3c(c2)C2(c4ccccc4-3)C3CC4CC(C3)CC2C4)cc1 ; c1ccc(-c2ccccc2-c2ccccc2-c2ccccc2N(c2cccc(-c3ccc4ccccc4c3)c2)c2ccc3c(c2)C2(c4ccccc4-3)C3CC4CC(C3)CC2C4)cc1 ; c1ccc(-c2ccccc2-c2ccccc2-c2ccccc2N(c2ccc(-c3cc4ccccc4c4ccccc34)cc2)c2ccc3c(c2)C2(c4ccccc4-3)C3CC4CC(C3)CC2C4)cc1 ; c1ccc(-c2ccccc2-c2ccccc2-c2ccccc2N(c2ccc(-c3ccc(-c4ccc5ccccc5c4)cc3)cc2)c2ccc3c(c2)C2(c4ccccc4-3)C3CC4CC(C3)CC2C4)cc1]

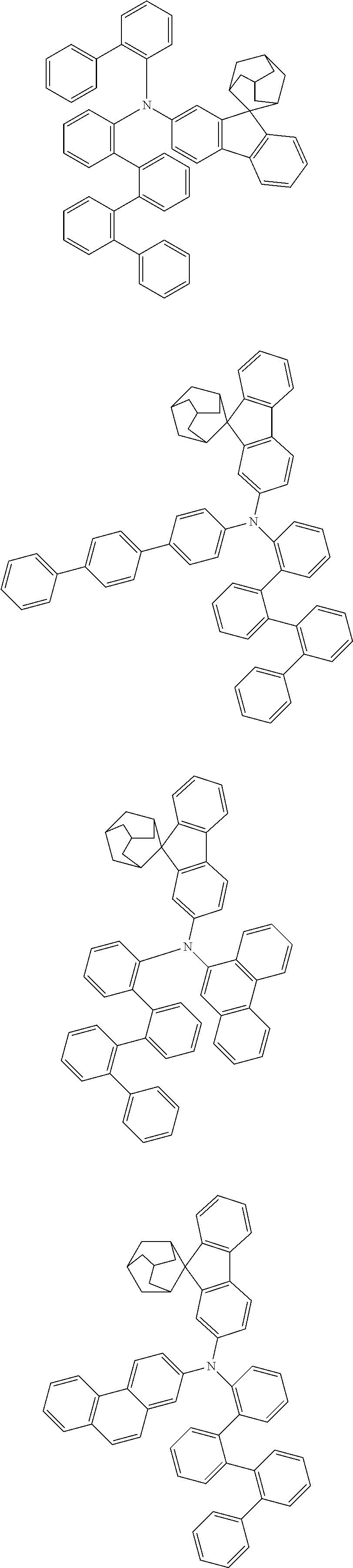 [SMILES: c1ccc(-c2ccccc2-c2ccccc2-c2ccccc2N(c2ccc3c(c2)C2(c4ccccc4-3)C3CC4CC(C3)CC2C4)c2ccccc2-c2ccccc2)cc1 ; c1ccc(-c2ccc(-c3ccc(N(c4ccc5c(c4)C4(c6ccccc6-5)C5CC6CC(C5)CC4C6)c4ccccc4-c4ccccc4-c4ccccc4-c4ccccc4)cc3)cc2)cc1 ; c1ccc(-c2ccccc2-c2ccccc2-c2ccccc2N(c2ccc3c(c2)C2(c4ccccc4-3)C3CC4CC(C3)CC2C4)c2cc3ccccc3c3ccccc23)cc1 ; c1ccc(-c2ccccc2-c2ccccc2-c2ccccc2N(c2ccc3c(c2)C2(c4ccccc4-3)C3CC4CC(C3)CC2C4)c2ccc3c(ccc4ccccc43)c2)cc1]

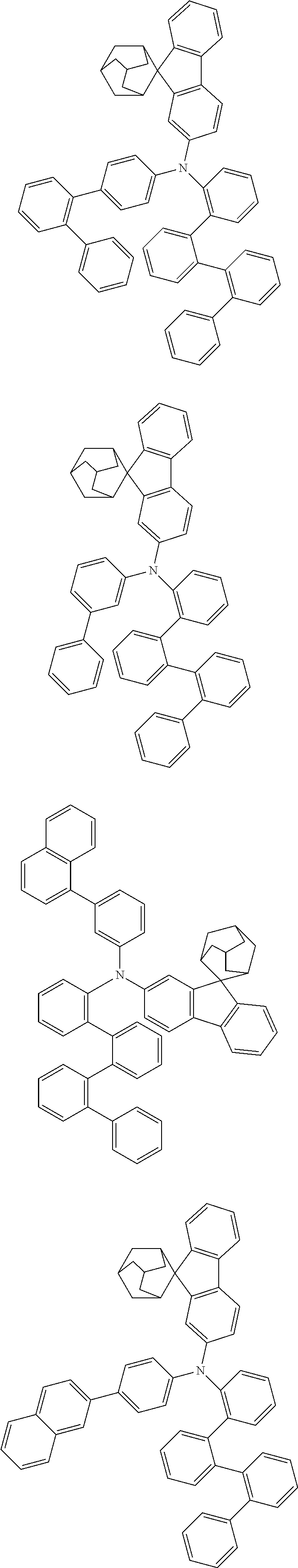 [SMILES: c1ccc(-c2ccccc2-c2ccc(N(c3ccc4c(c3)C3(c5ccccc5-4)C4CC5CC(C4)CC3C5)c3ccccc3-c3ccccc3-c3ccccc3-c3ccccc3)cc2)cc1 ; c1ccc(-c2cccc(N(c3ccc4c(c3)C3(c5ccccc5-4)C4CC5CC(C4)CC3C5)c3ccccc3-c3ccccc3-c3ccccc3-c3ccccc3)c2)cc1 ; c1ccc(-c2ccccc2-c2ccccc2-c2ccccc2N(c2cccc(-c3cccc4ccccc34)c2)c2ccc3c(c2)C2(c4ccccc4-3)C3CC4CC(C3)CC2C4)cc1 ; c1ccc(-c2ccccc2-c2ccccc2-c2ccccc2N(c2ccc(-c3ccc4ccccc4c3)cc2)c2ccc3c(c2)C2(c4ccccc4-3)C3CC4CC(C3)CC2C4)cc1]

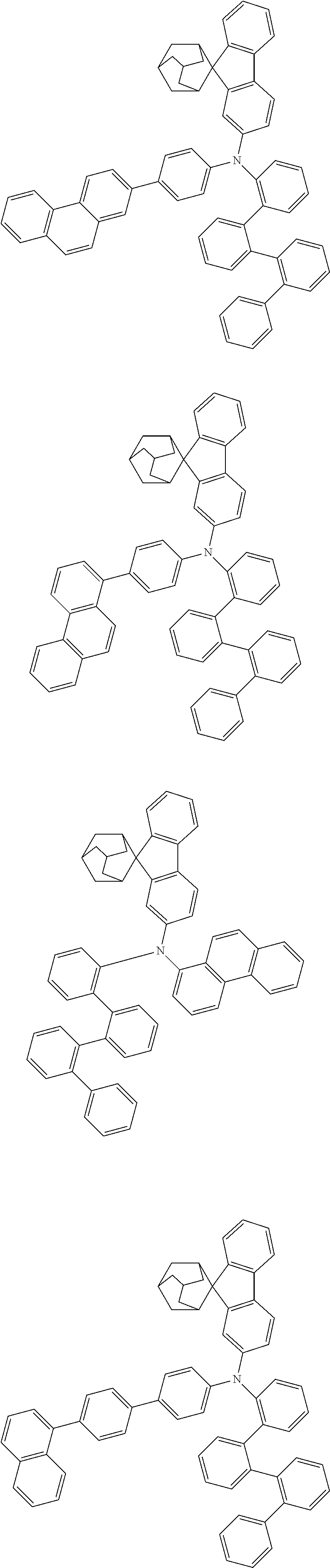 [SMILES: c1ccc(-c2ccccc2-c2ccccc2-c2ccccc2N(c2ccc(-c3ccc4c(ccc5ccccc54)c3)cc2)c2ccc3c(c2)C2(c4ccccc4-3)C3CC4CC(C3)CC2C4)cc1 ; c1ccc(-c2ccccc2-c2ccccc2-c2ccccc2N(c2ccc(-c3cccc4c3ccc3ccccc34)cc2)c2ccc3c(c2)C2(c4ccccc4-3)C3CC4CC(C3)CC2C4)cc1 ; c1ccc(-c2ccccc2-c2ccccc2-c2ccccc2N(c2ccc3c(c2)C2(c4ccccc4-3)C3CC4CC(C3)CC2C4)c2cccc3c2ccc2ccccc23)cc1 ; c1ccc(-c2ccccc2-c2ccccc2-c2ccccc2N(c2ccc(-c3ccc(-c4cccc5ccccc45)cc3)cc2)c2ccc3c(c2)C2(c4ccccc4-3)C3CC4CC(C3)CC2C4)cc1]

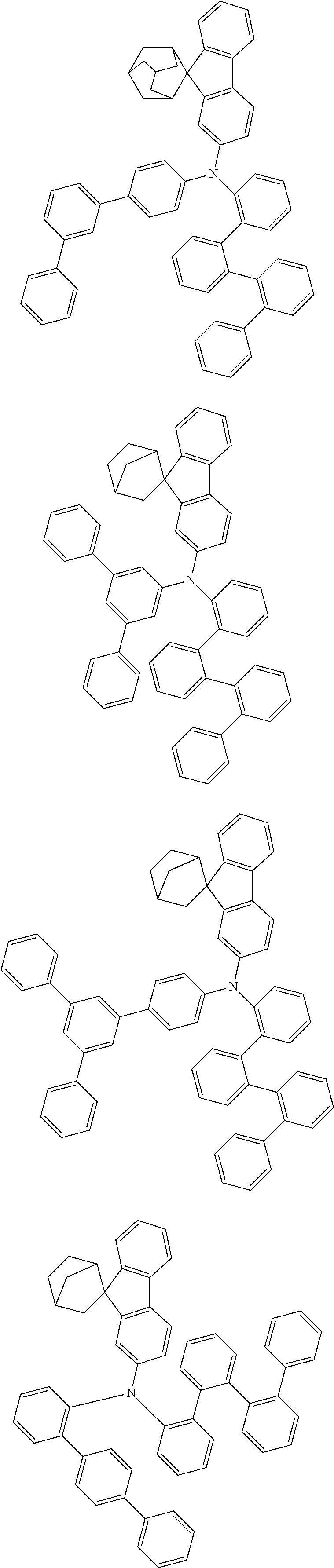

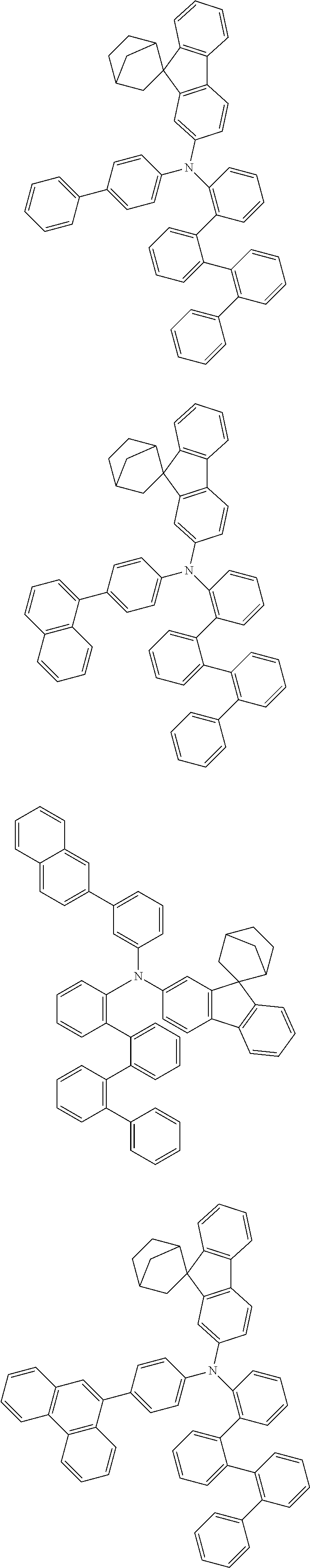 [SMILES: c1ccc(-c2ccc(N(c3ccc4c(c3)C3(CC5CCC3C5)c3ccccc3-4)c3ccccc3-c3ccccc3-c3ccccc3-c3ccccc3)cc2)cc1 ; c1ccc(-c2ccccc2-c2ccccc2-c2ccccc2N(c2ccc(-c3cccc4ccccc34)cc2)c2ccc3c(c2)C2(CC4CCC2C4)c2ccccc2-3)cc1 ; c1ccc(-c2ccccc2-c2ccccc2-c2ccccc2N(c2cccc(-c3ccc4ccccc4c3)c2)c2ccc3c(c2)C2(CC4CCC2C4)c2ccccc2-3)cc1 ; c1ccc(-c2ccccc2-c2ccccc2-c2ccccc2N(c2ccc(-c3cc4ccccc4c4ccccc34)cc2)c2ccc3c(c2)C2(CC4CCC2C4)c2ccccc2-3)cc1]

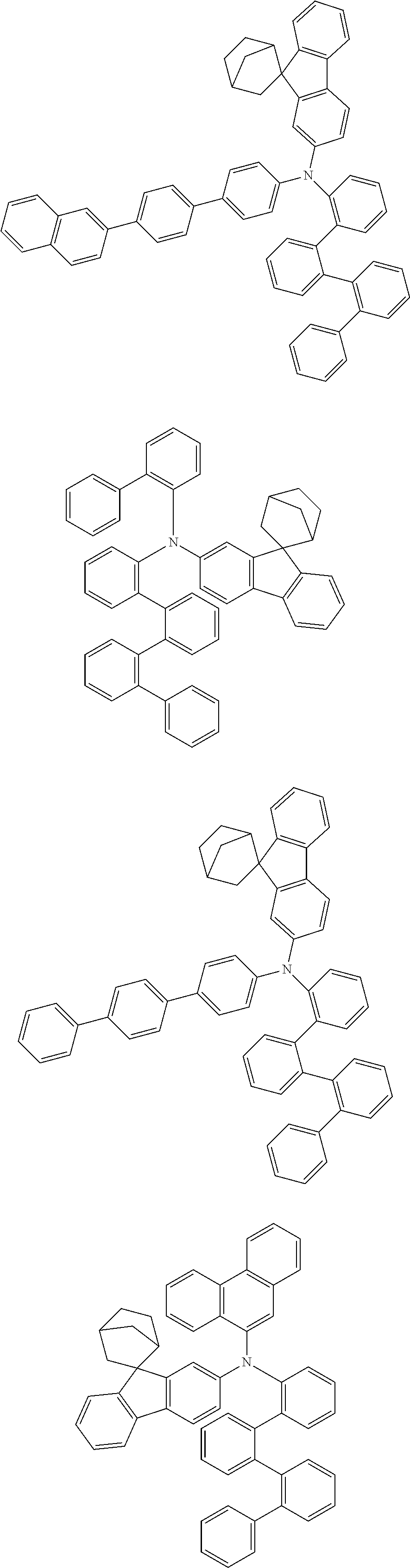 [SMILES: c1ccc(-c2ccccc2-c2ccccc2-c2ccccc2N(c2ccc(-c3ccc(-c4ccc5ccccc5c4)cc3)cc2)c2ccc3c(c2)C2(CC4CCC2C4)c2ccccc2-3)cc1 ; c1ccc(-c2ccccc2-c2ccccc2-c2ccccc2N(c2ccc3c(c2)C2(CC4CCC2C4)c2ccccc2-3)c2ccccc2-c2ccccc2)cc1 ; c1ccc(-c2ccc(-c3ccc(N(c4ccc5c(c4)C4(CC6CCC4C6)c4ccccc4-5)c4ccccc4-c4ccccc4-c4ccccc4-c4ccccc4)cc3)cc2)cc1 ; c1ccc(-c2ccccc2-c2ccccc2-c2ccccc2N(c2ccc3c(c2)C2(CC4CCC2C4)c2ccccc2-3)c2cc3ccccc3c3ccccc23)cc1]

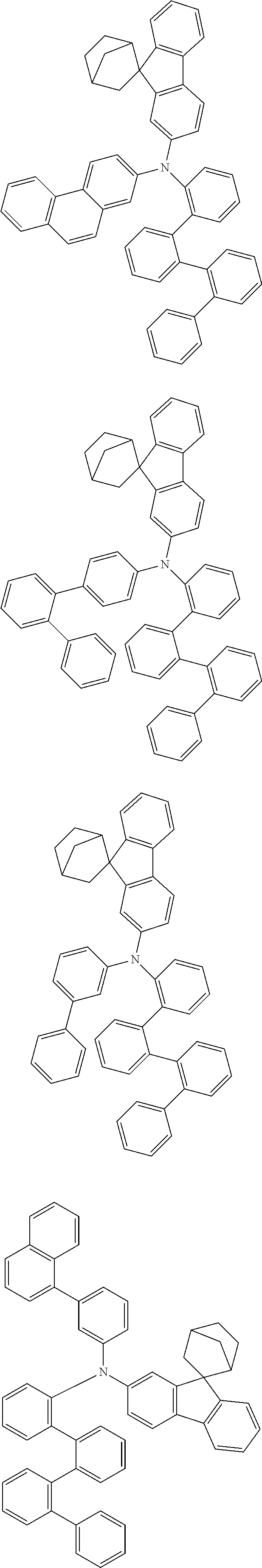

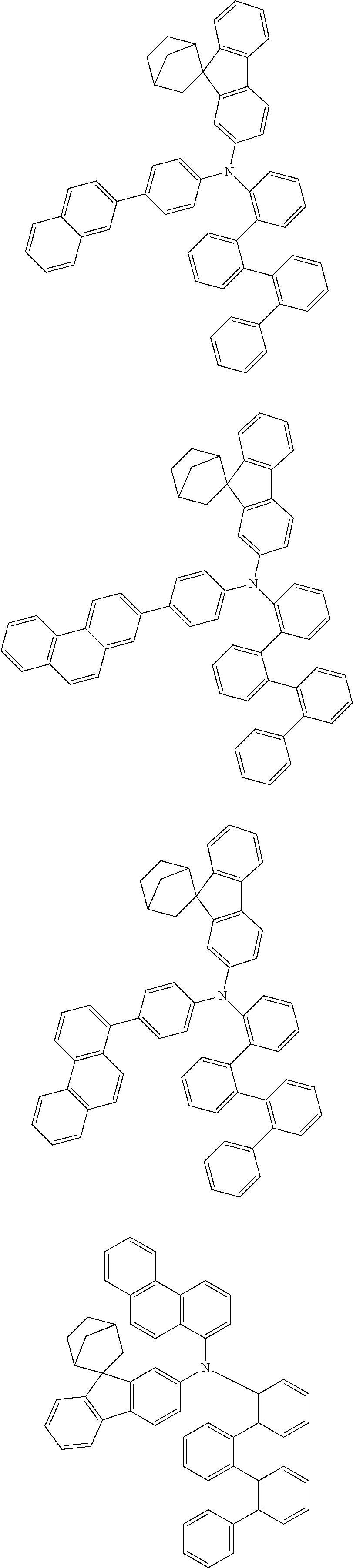 [SMILES: c1ccc(-c2ccccc2-c2ccccc2-c2ccccc2N(c2ccc(-c3ccc4ccccc4c3)cc2)c2ccc3c(c2)C2(CC4CCC2C4)c2ccccc2-3)cc1 ; c1ccc(-c2ccccc2-c2ccccc2-c2ccccc2N(c2ccc(-c3ccc4c(ccc5ccccc54)c3)cc2)c2ccc3c(c2)C2(CC4CCC2C4)c2ccccc2-3)cc1 ; c1ccc(-c2ccccc2-c2ccccc2-c2ccccc2N(c2ccc(-c3cccc4c3ccc3ccccc34)cc2)c2ccc3c(c2)C2(CC4CCC2C4)c2ccccc2-3)cc1 ; c1ccc(-c2ccccc2-c2ccccc2-c2ccccc2N(c2ccc3c(c2)C2(CC4CCC2C4)c2ccccc2-3)c2cccc3c2ccc2ccccc23)cc1]

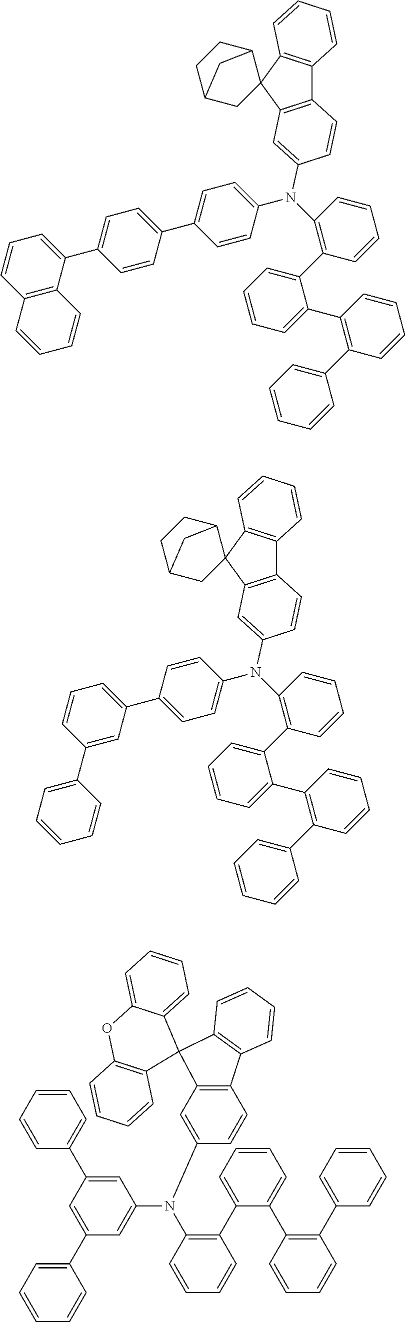 [SMILES: c1ccc(-c2ccccc2-c2ccccc2-c2ccccc2N(c2ccc(-c3ccc(-c4cccc5ccccc45)cc3)cc2)c2ccc3c(c2)C2(CC4CCC2C4)c2ccccc2-3)cc1 ; c1ccc(-c2cccc(-c3ccc(N(c4ccc5c(c4)C4(CC6CCC4C6)c4ccccc4-5)c4ccccc4-c4ccccc4-c4ccccc4-c4ccccc4)cc3)c2)cc1 ; c1ccc(-c2cc(-c3ccccc3)cc(N(c3ccc4c(c3)C3(c5ccccc5Oc5ccccc53)c3ccccc3-4)c3ccccc3-c3ccccc3-c3ccccc3-c3ccccc3)c2)cc1]

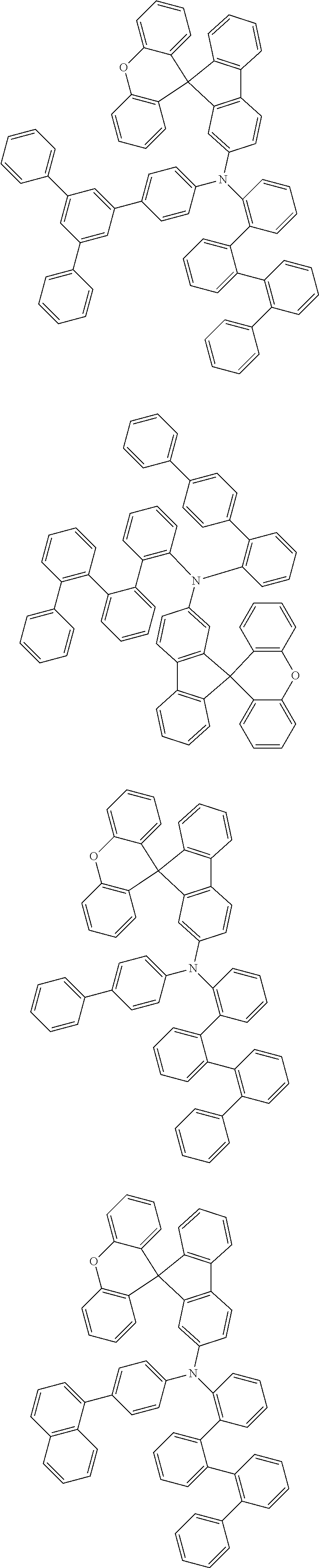 [SMILES: c1ccc(-c2cc(-c3ccccc3)cc(-c3ccc(N(c4ccc5c(c4)C4(c6ccccc6Oc6ccccc64)c4ccccc4-5)c4ccccc4-c4ccccc4-c4ccccc4-c4ccccc4)cc3)c2)cc1 ; c1ccc(-c2ccc(-c3ccccc3N(c3ccc4c(c3)C3(c5ccccc5Oc5ccccc53)c3ccccc3-4)c3ccccc3-c3ccccc3-c3ccccc3-c3ccccc3)cc2)cc1 ; c1ccc(-c2ccc(N(c3ccc4c(c3)C3(c5ccccc5Oc5ccccc53)c3ccccc3-4)c3ccccc3-c3ccccc3-c3ccccc3-c3ccccc3)cc2)cc1 ; c1ccc(-c2ccccc2-c2ccccc2-c2ccccc2N(c2ccc(-c3cccc4ccccc34)cc2)c2ccc3c(c2)C2(c4ccccc4Oc4ccccc42)c2ccccc2-3)cc1]

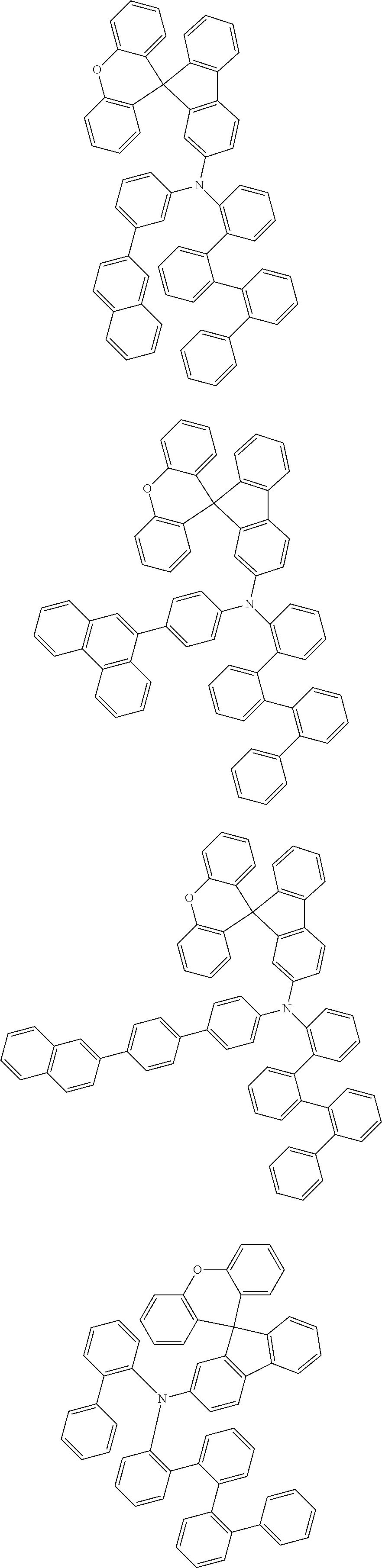

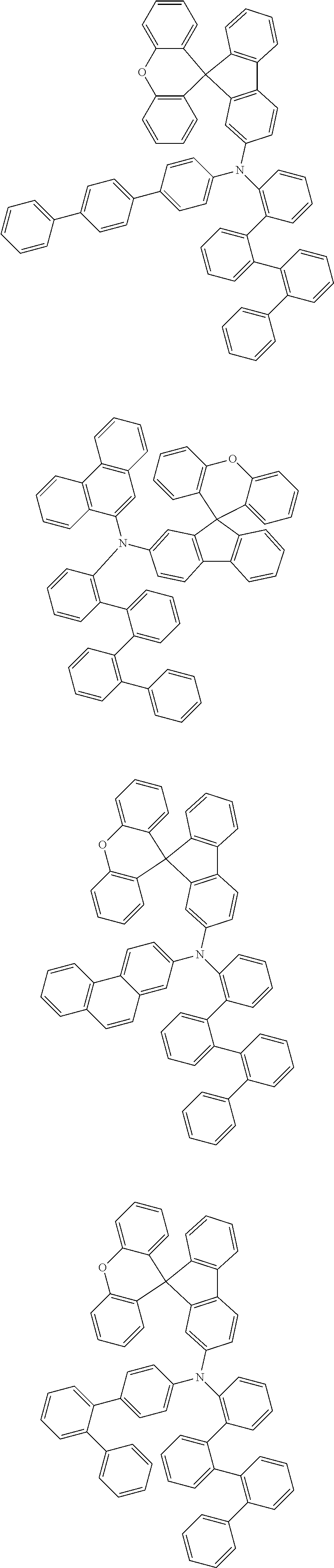 [SMILES: c1ccc(-c2ccc(-c3ccc(N(c4ccc5c(c4)C4(c6ccccc6Oc6ccccc64)c4ccccc4-5)c4ccccc4-c4ccccc4-c4ccccc4-c4ccccc4)cc3)cc2)cc1 ; c1ccc(-c2ccccc2-c2ccccc2-c2ccccc2N(c2ccc3c(c2)C2(c4ccccc4Oc4ccccc42)c2ccccc2-3)c2cc3ccccc3c3ccccc23)cc1 ; c1ccc(-c2ccccc2-c2ccccc2-c2ccccc2N(c2ccc3c(c2)C2(c4ccccc4Oc4ccccc42)c2ccccc2-3)c2ccc3c(ccc4ccccc43)c2)cc1 ; c1ccc(-c2ccccc2-c2ccc(N(c3ccc4c(c3)C3(c5ccccc5Oc5ccccc53)c3ccccc3-4)c3ccccc3-c3ccccc3-c3ccccc3-c3ccccc3)cc2)cc1]

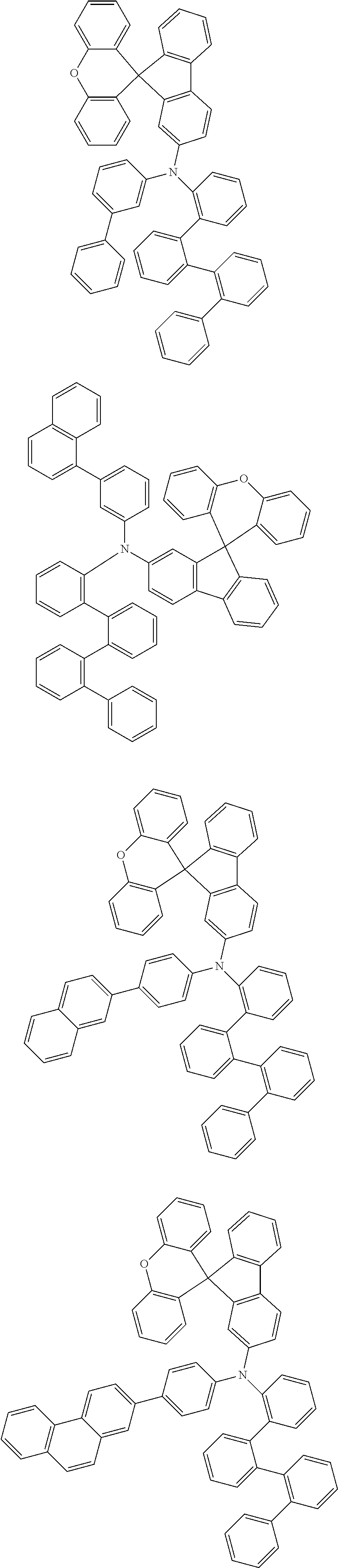 [SMILES: c1ccc(-c2cccc(N(c3ccc4c(c3)C3(c5ccccc5Oc5ccccc53)c3ccccc3-4)c3ccccc3-c3ccccc3-c3ccccc3-c3ccccc3)c2)cc1 ; c1ccc(-c2ccccc2-c2ccccc2-c2ccccc2N(c2cccc(-c3cccc4ccccc34)c2)c2ccc3c(c2)C2(c4ccccc4Oc4ccccc42)c2ccccc2-3)cc1 ; c1ccc(-c2ccccc2-c2ccccc2-c2ccccc2N(c2ccc(-c3ccc4ccccc4c3)cc2)c2ccc3c(c2)C2(c4ccccc4Oc4ccccc42)c2ccccc2-3)cc1 ; c1ccc(-c2ccccc2-c2ccccc2-c2ccccc2N(c2ccc(-c3ccc4c(ccc5ccccc54)c3)cc2)c2ccc3c(c2)C2(c4ccccc4Oc4ccccc42)c2ccccc2-3)cc1]

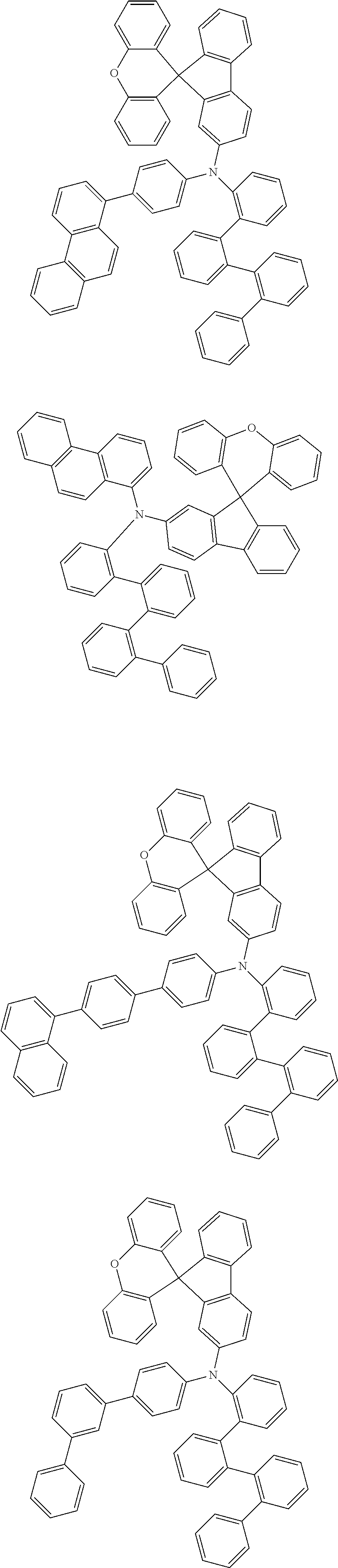 [SMILES: c1ccc(-c2ccccc2-c2ccccc2-c2ccccc2N(c2ccc(-c3cccc4c3ccc3ccccc34)cc2)c2ccc3c(c2)C2(c4ccccc4Oc4ccccc42)c2ccccc2-3)cc1 ; c1ccc(-c2ccccc2-c2ccccc2-c2ccccc2N(c2ccc3c(c2)C2(c4ccccc4Oc4ccccc42)c2ccccc2-3)c2cccc3c2ccc2ccccc23)cc1 ; c1ccc(-c2ccccc2-c2ccccc2-c2ccccc2N(c2ccc(-c3ccc(-c4cccc5ccccc45)cc3)cc2)c2ccc3c(c2)C2(c4ccccc4Oc4ccccc42)c2ccccc2-3)cc1 ; c1ccc(-c2cccc(-c3ccc(N(c4ccc5c(c4)C4(c6ccccc6Oc6ccccc64)c4ccccc4-5)c4ccccc4-c4ccccc4-c4ccccc4-c4ccccc4)cc3)c2)cc1]

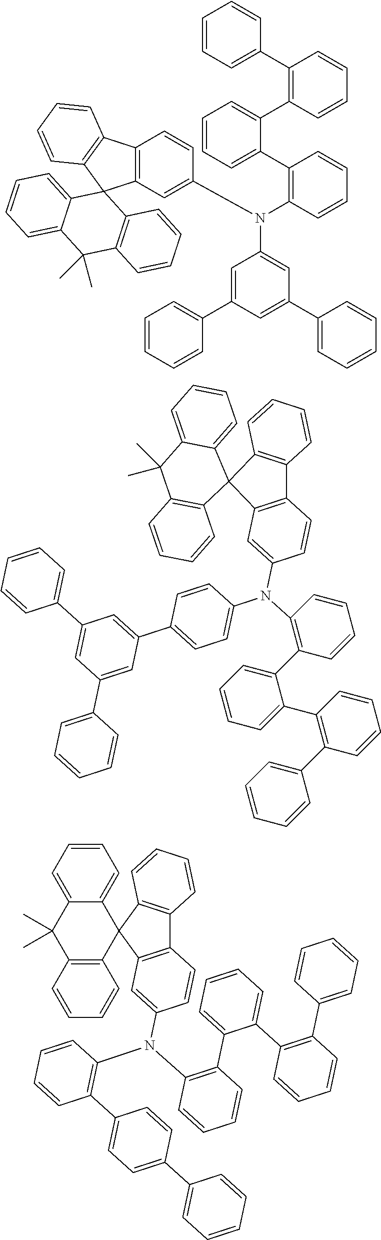

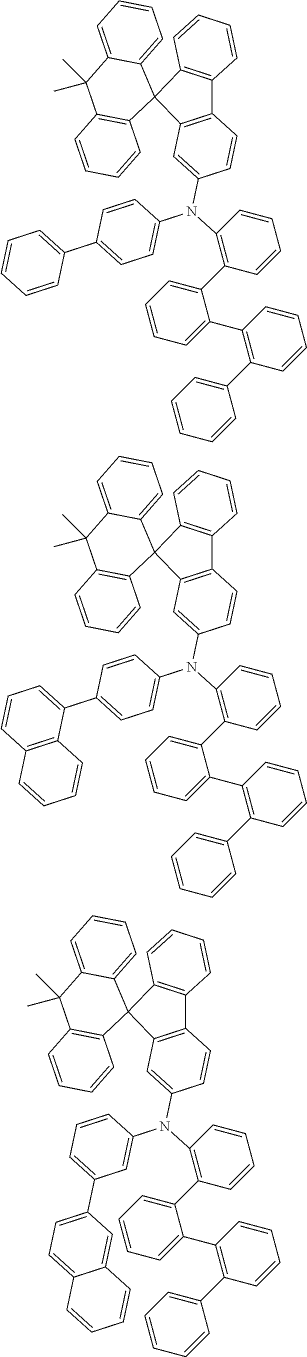

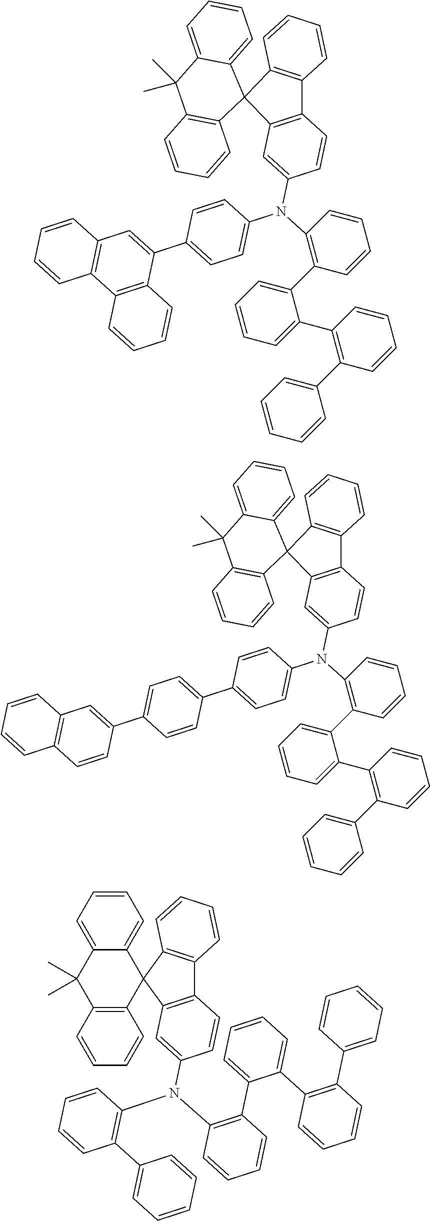

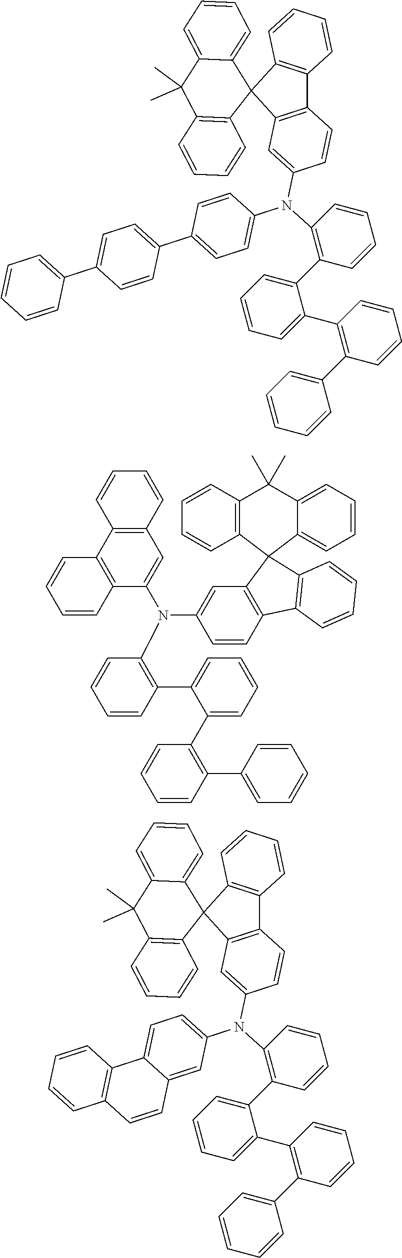

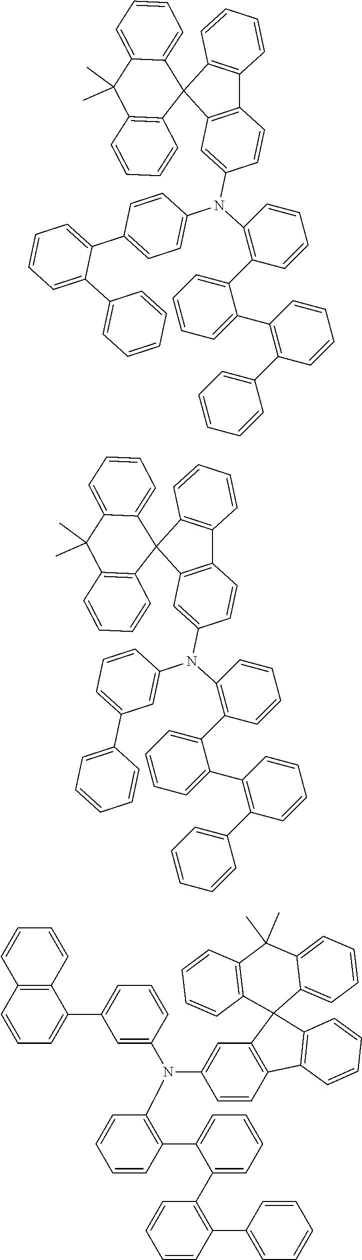

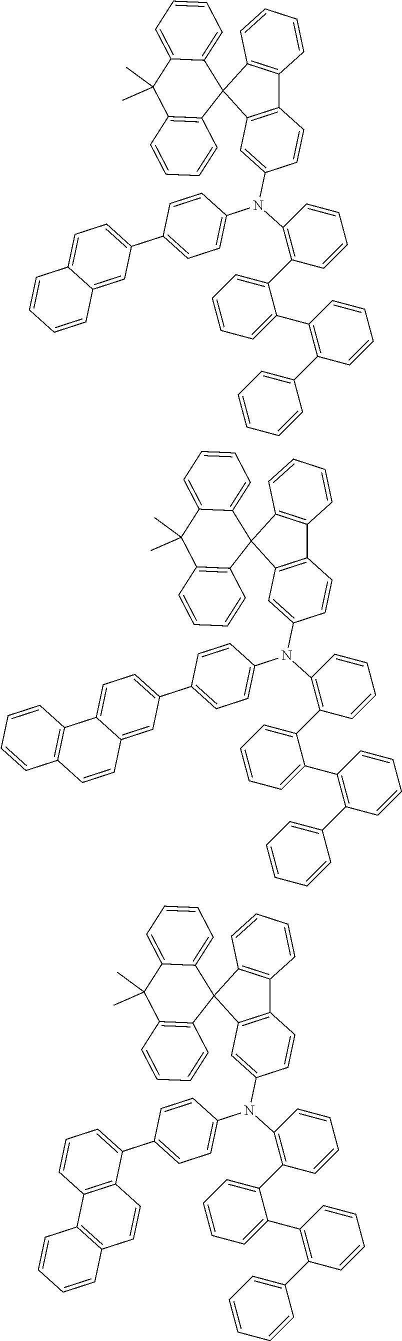

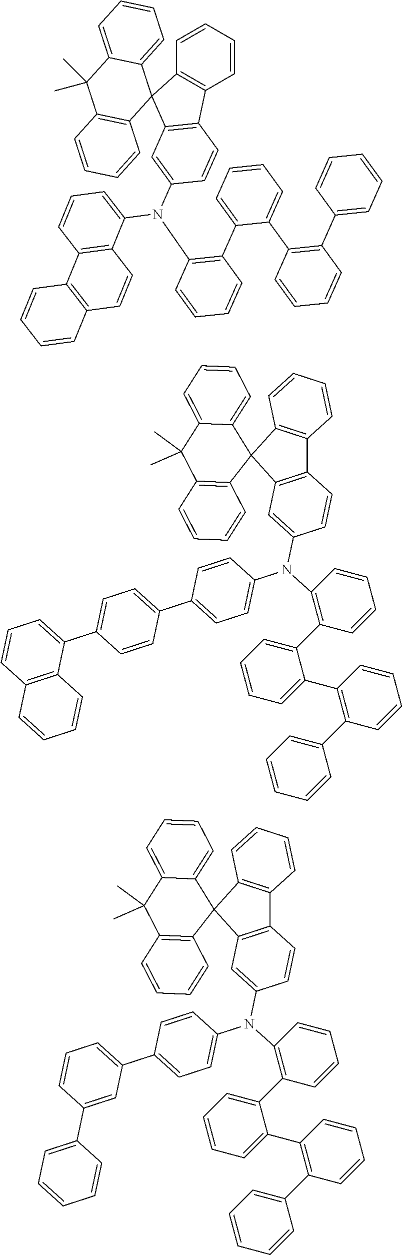

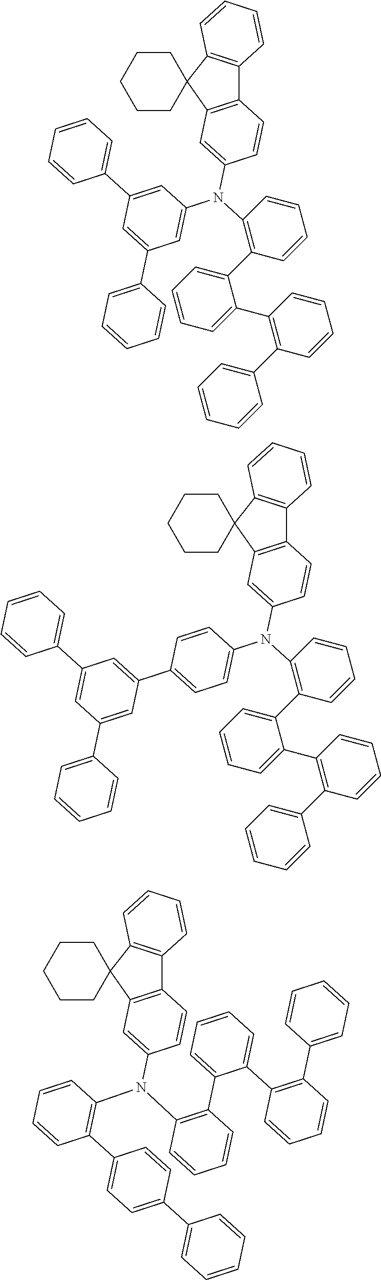

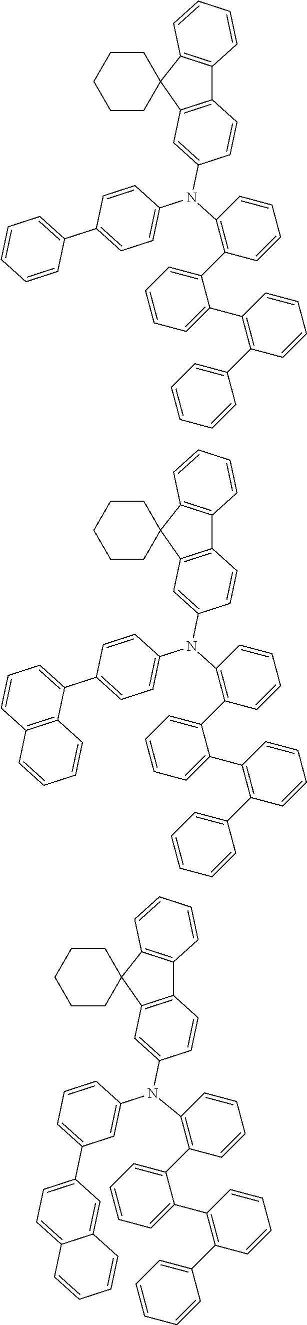

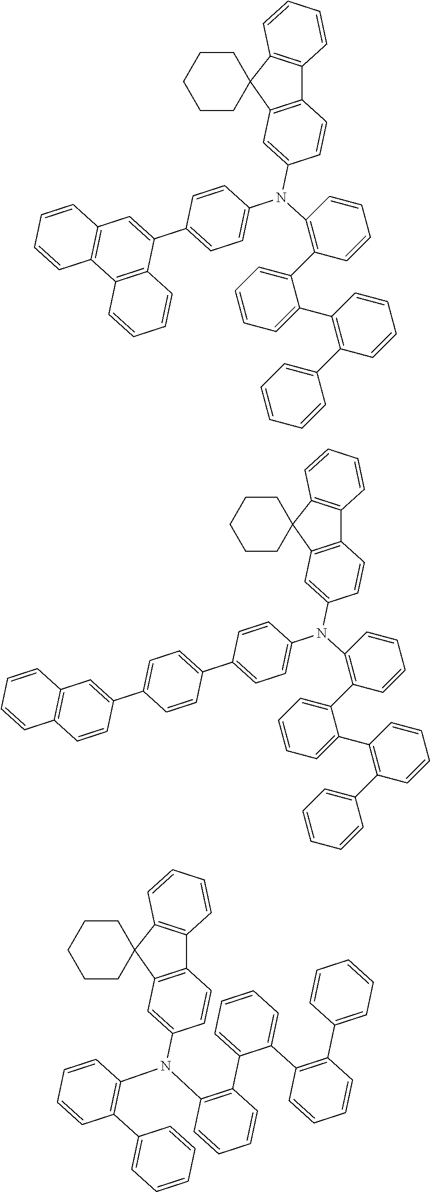

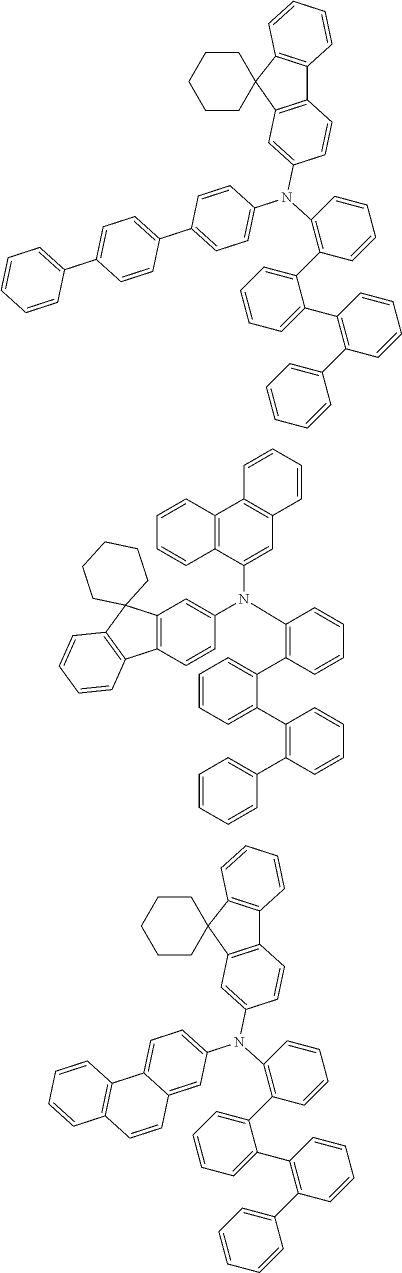

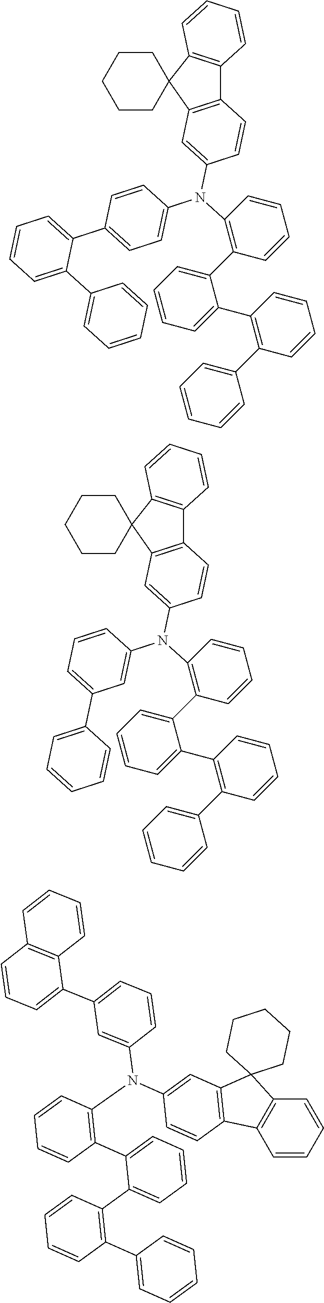

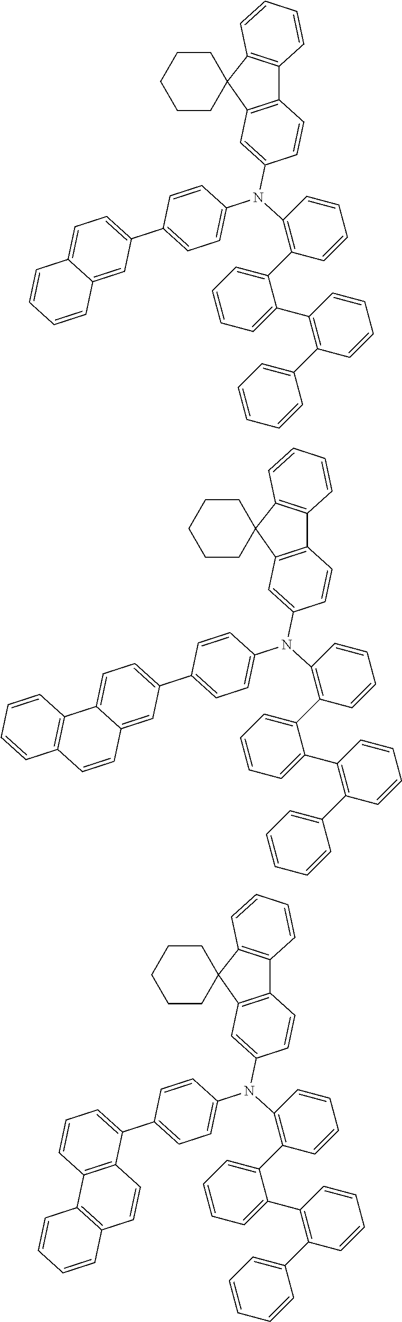

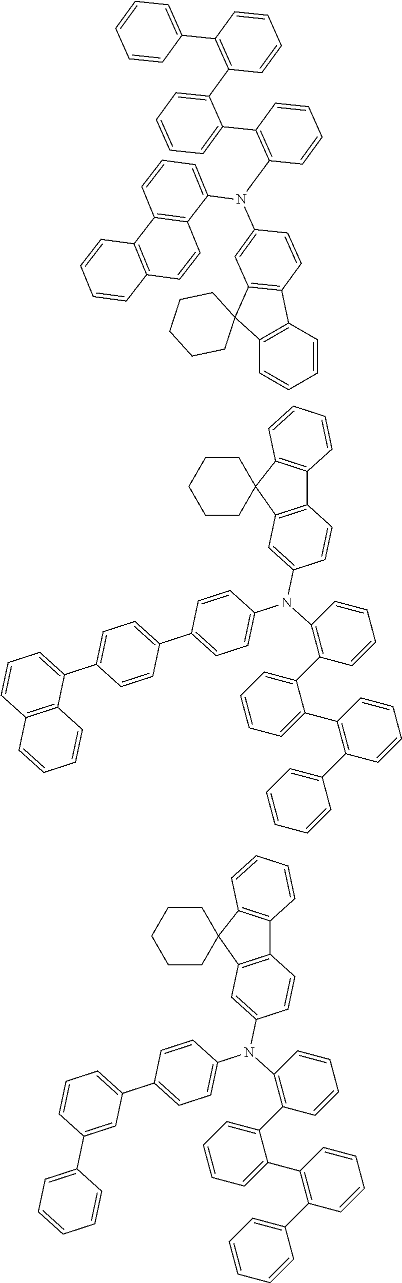

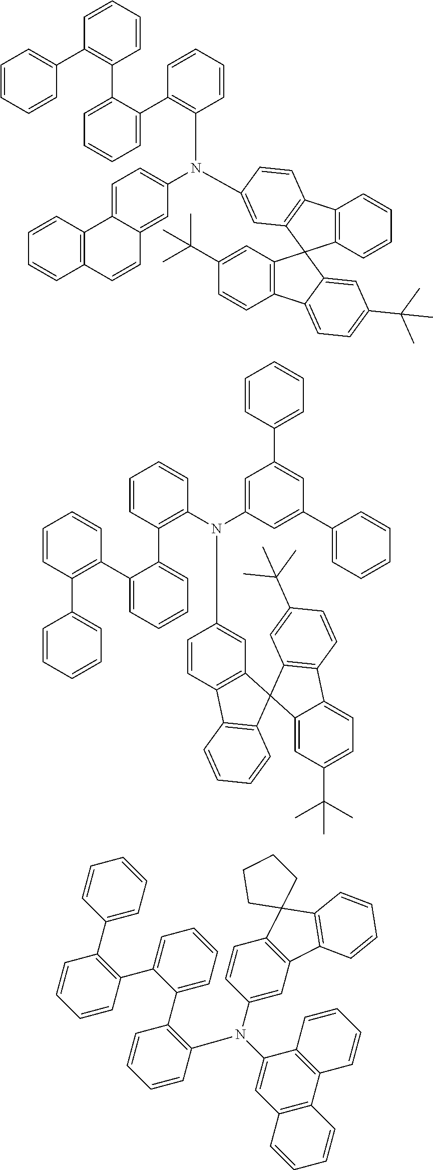

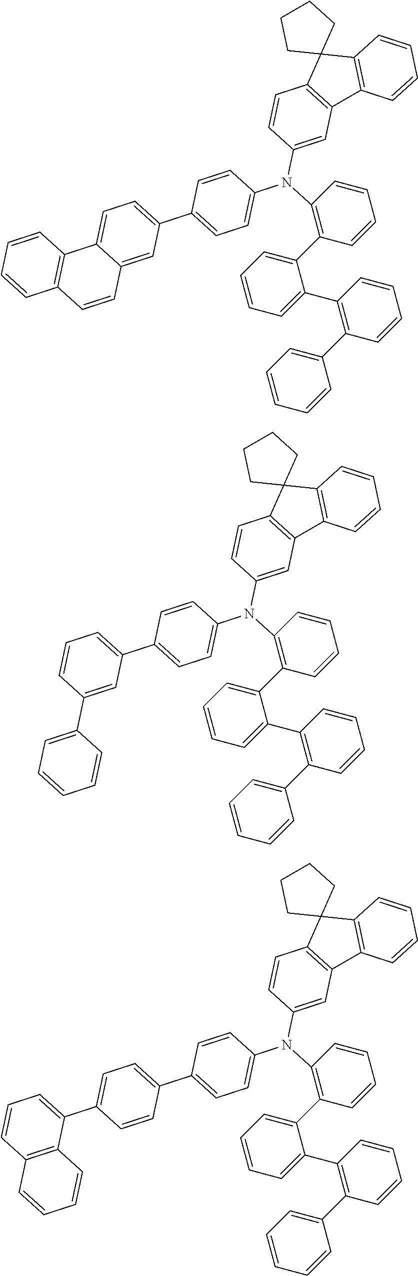

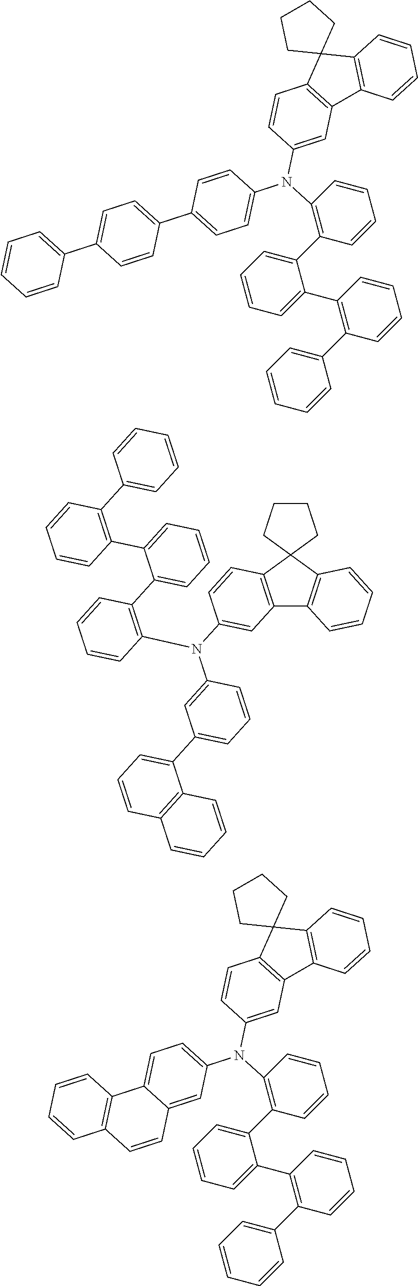

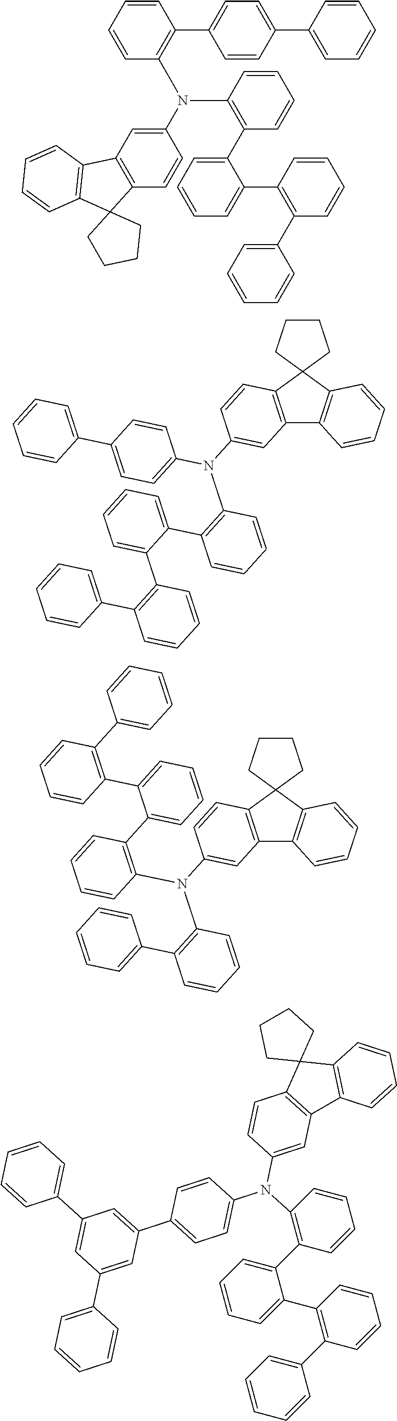

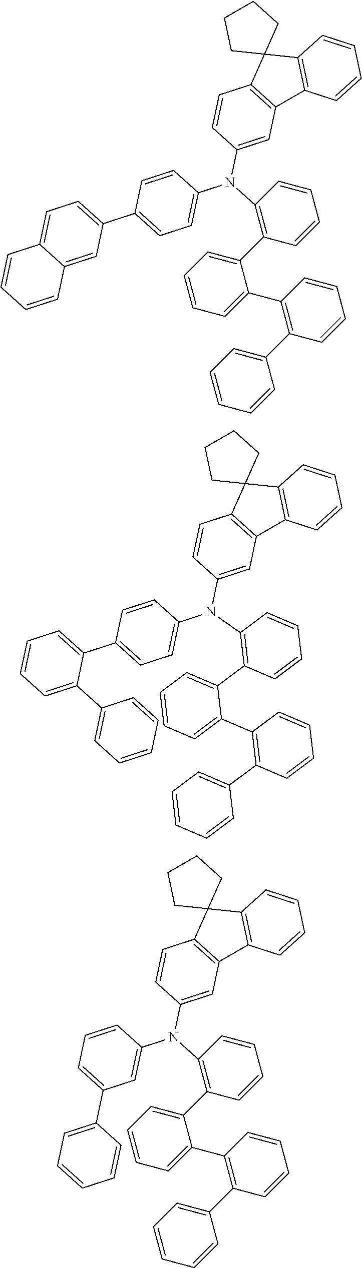

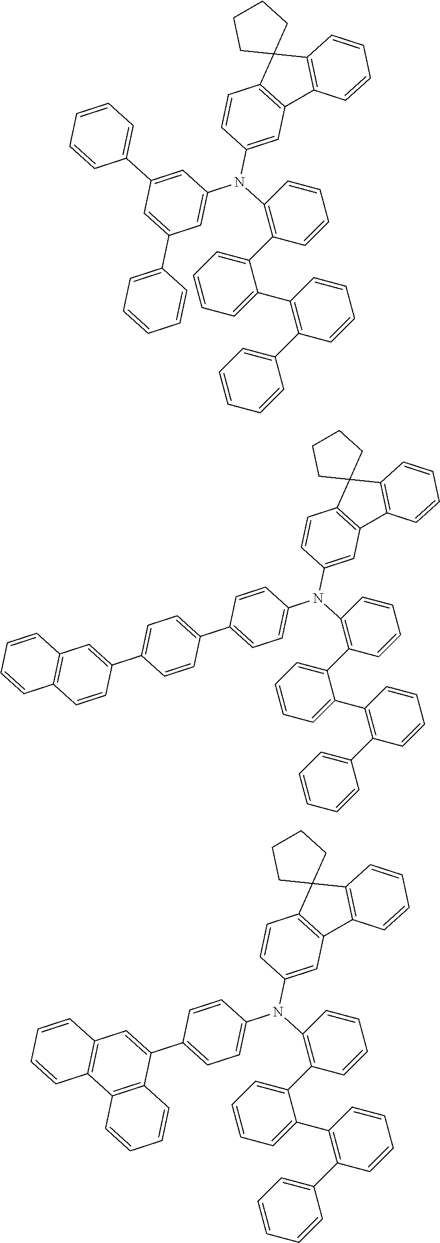

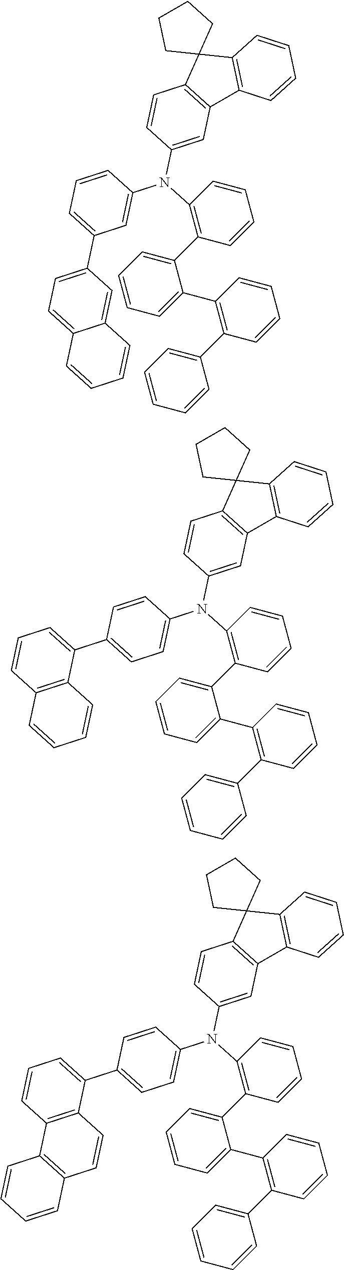

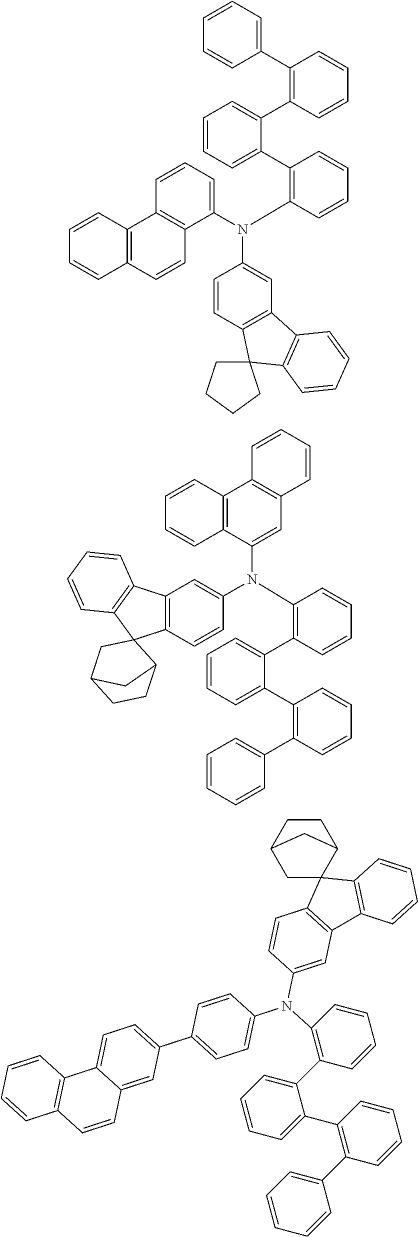

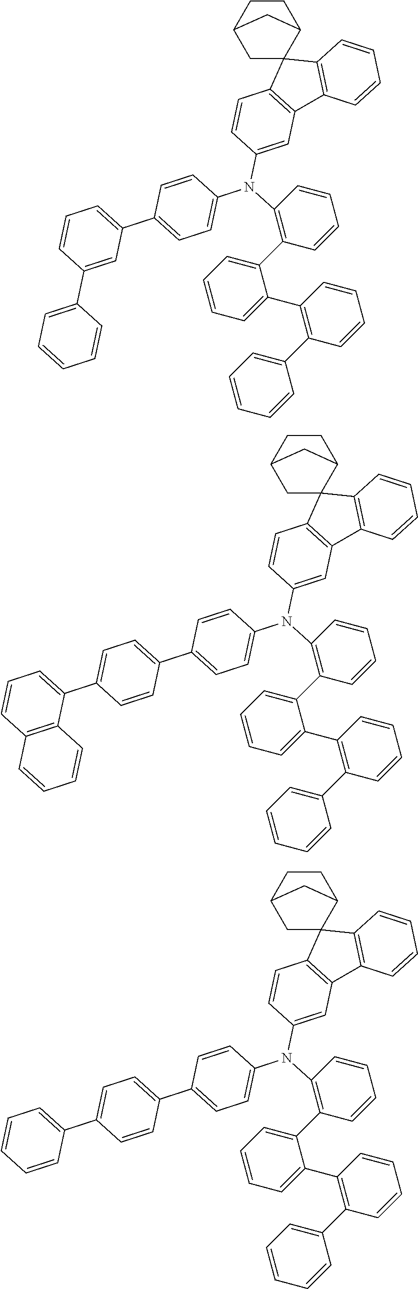

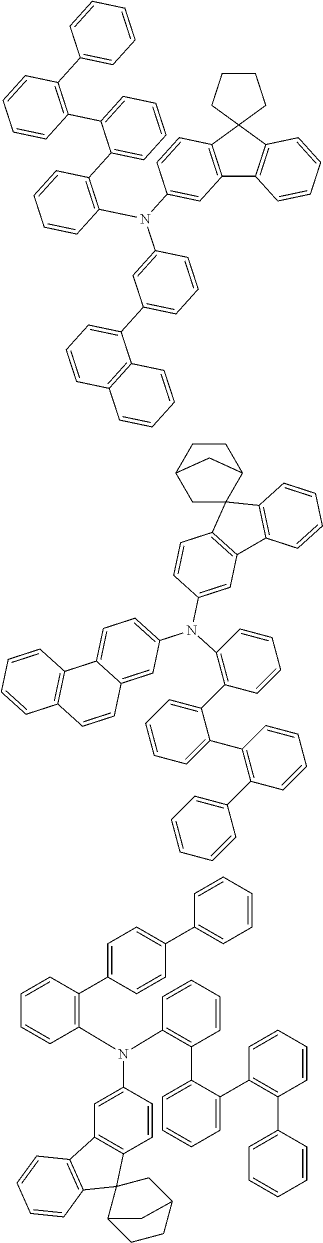

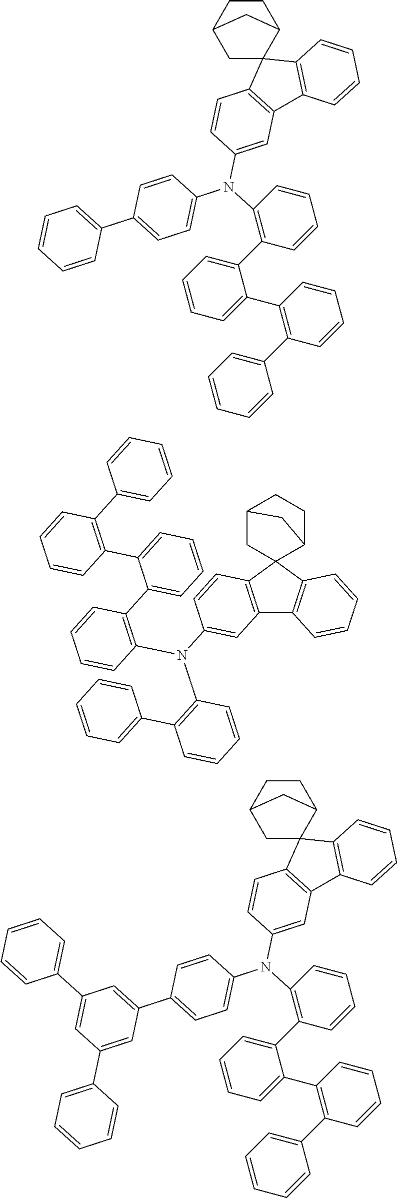 [SMILES: c1ccc(-c2ccc(N(c3ccc4c(c3)-c3ccccc3C43CC4CCC3C4)c3ccccc3-c3ccccc3-c3ccccc3-c3ccccc3)cc2)cc1 ; c1ccc(-c2ccccc2-c2ccccc2-c2ccccc2N(c2ccc3c(c2)-c2ccccc2C32CC3CCC2C3)c2ccccc2-c2ccccc2)cc1 ; c1ccc(-c2cc(-c3ccccc3)cc(-c3ccc(N(c4ccc5c(c4)-c4ccccc4C54CC5CCC4C5)c4ccccc4-c4ccccc4-c4ccccc4-c4ccccc4)cc3)c2)cc1]

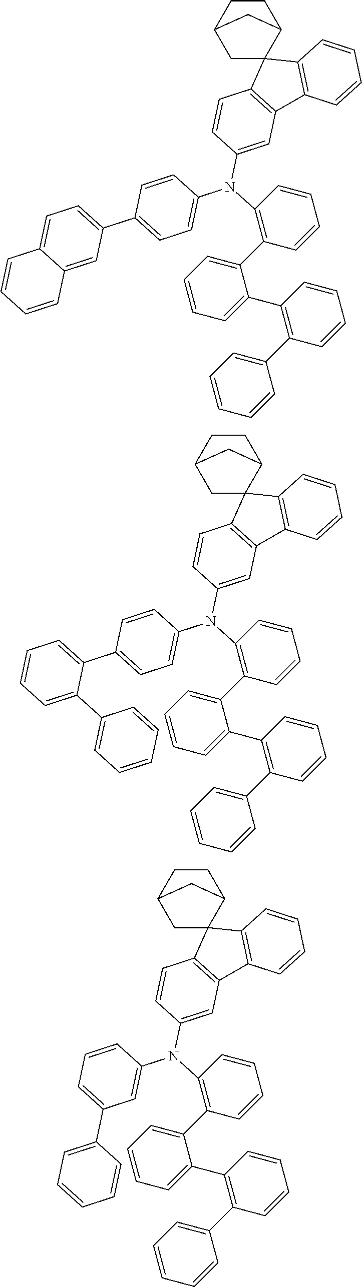

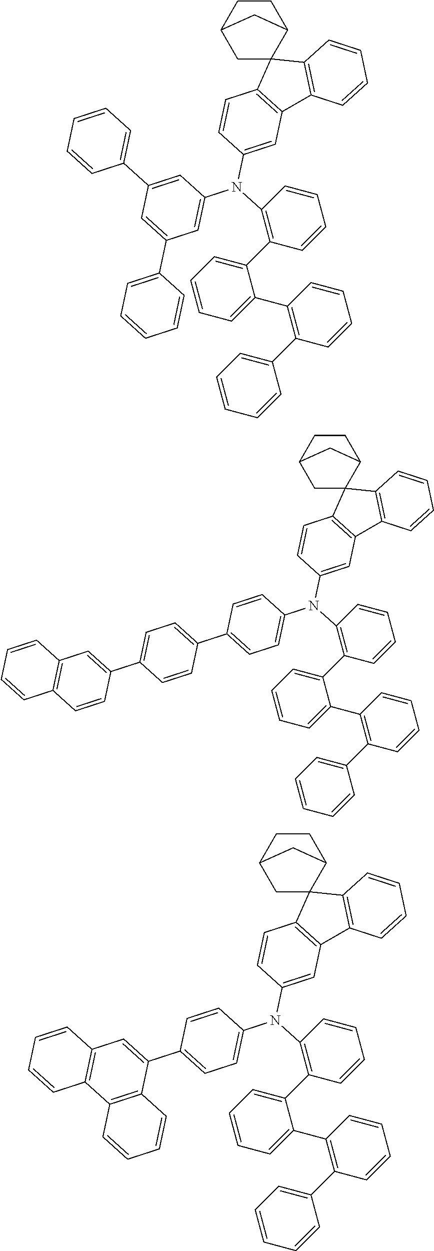

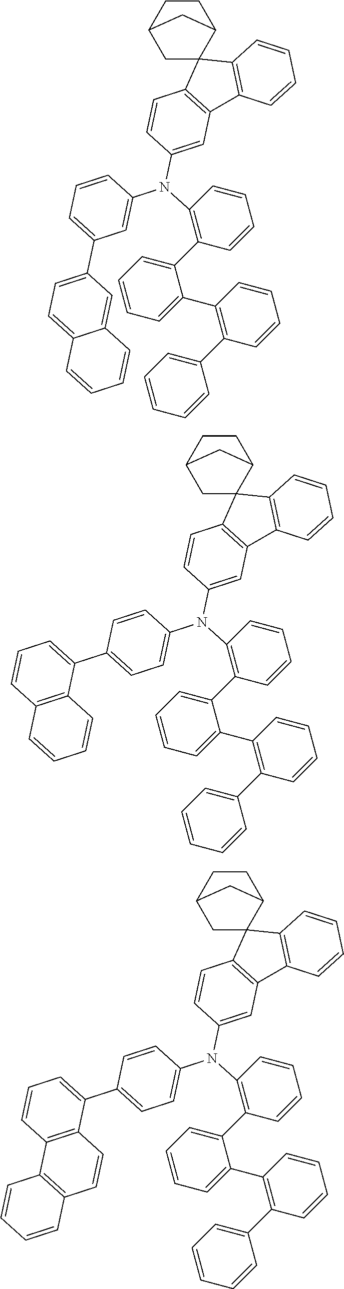

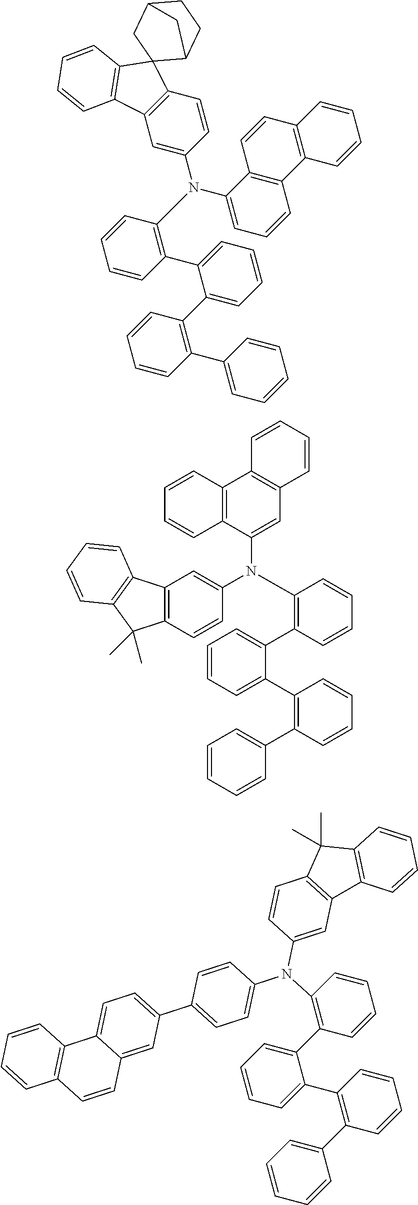

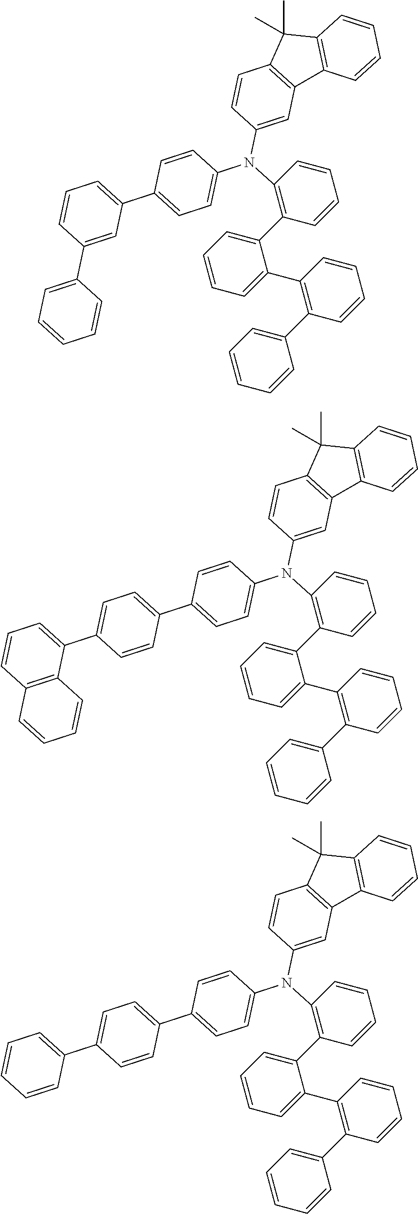

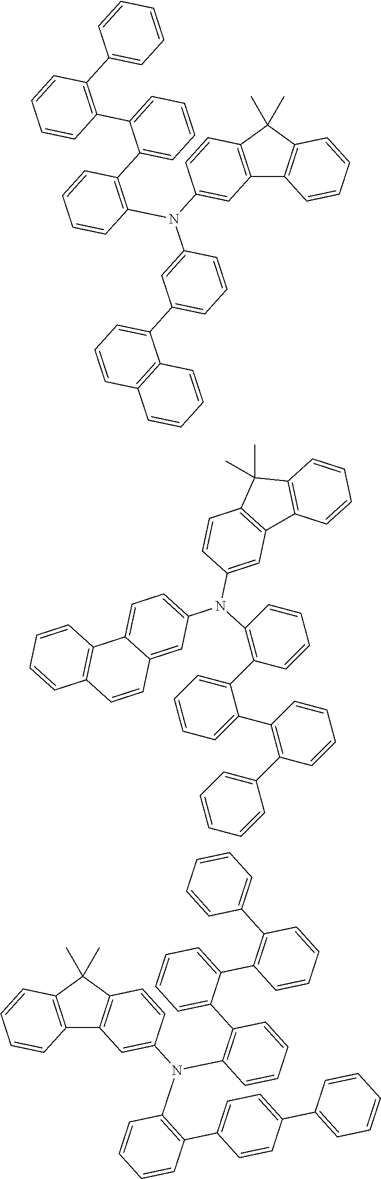

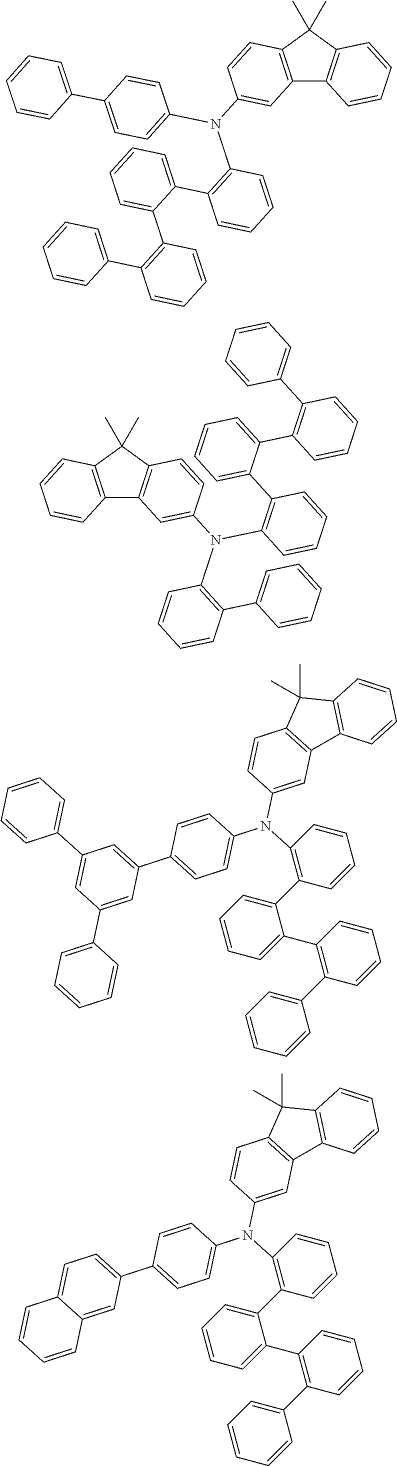

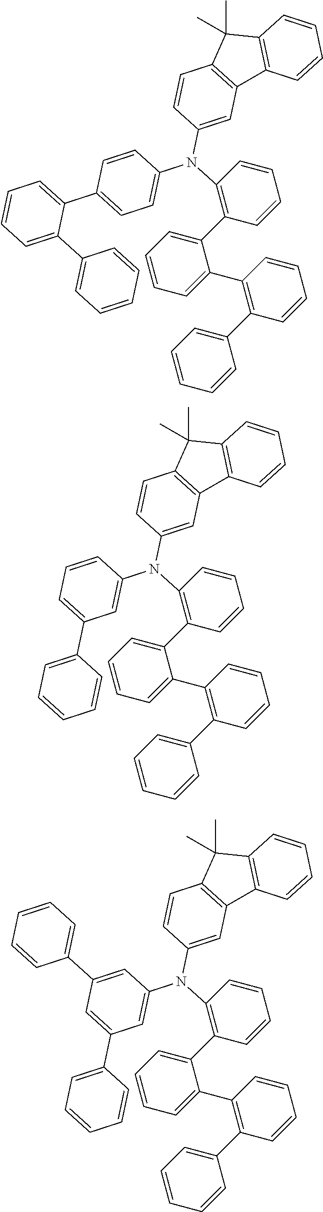

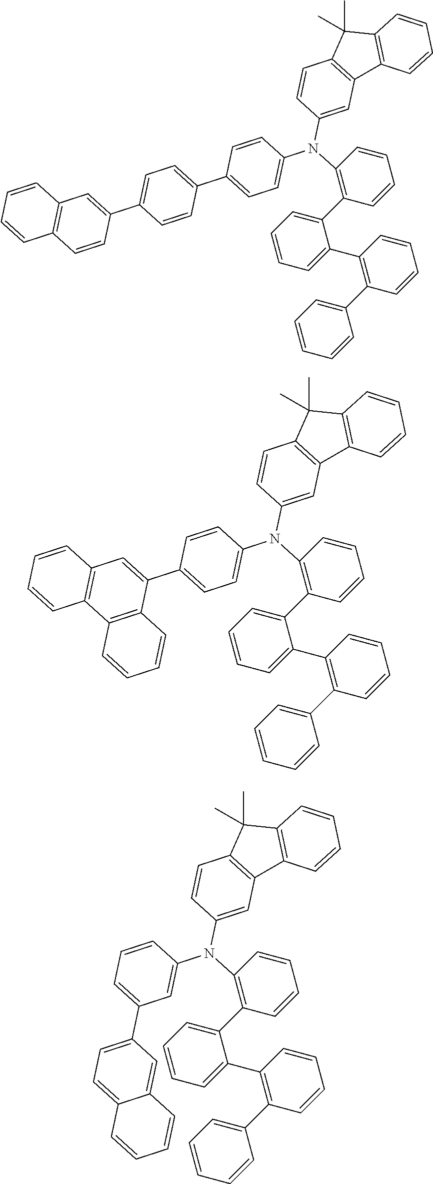

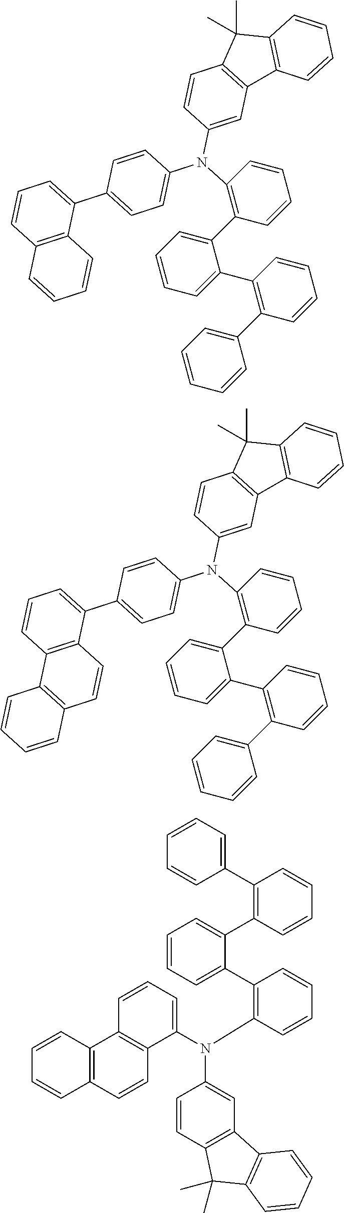

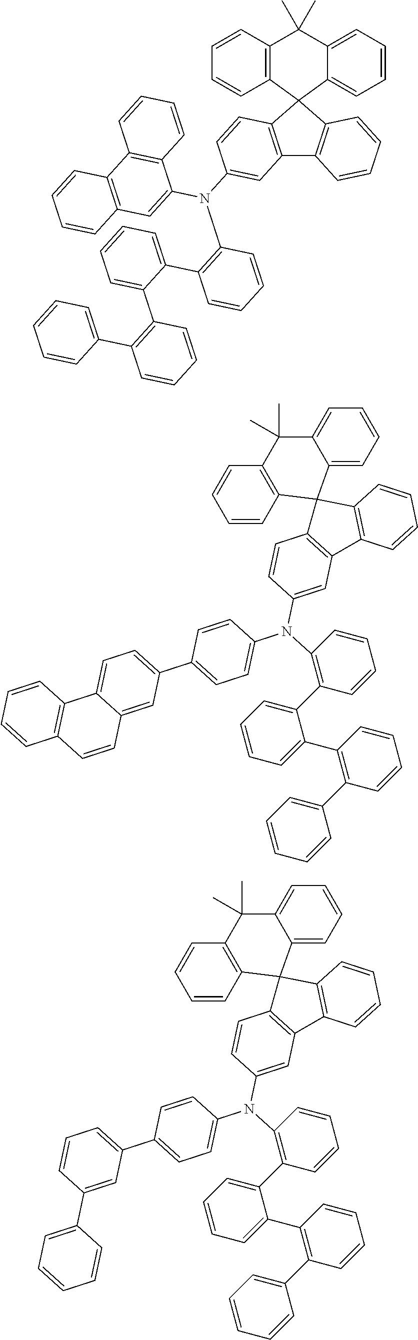

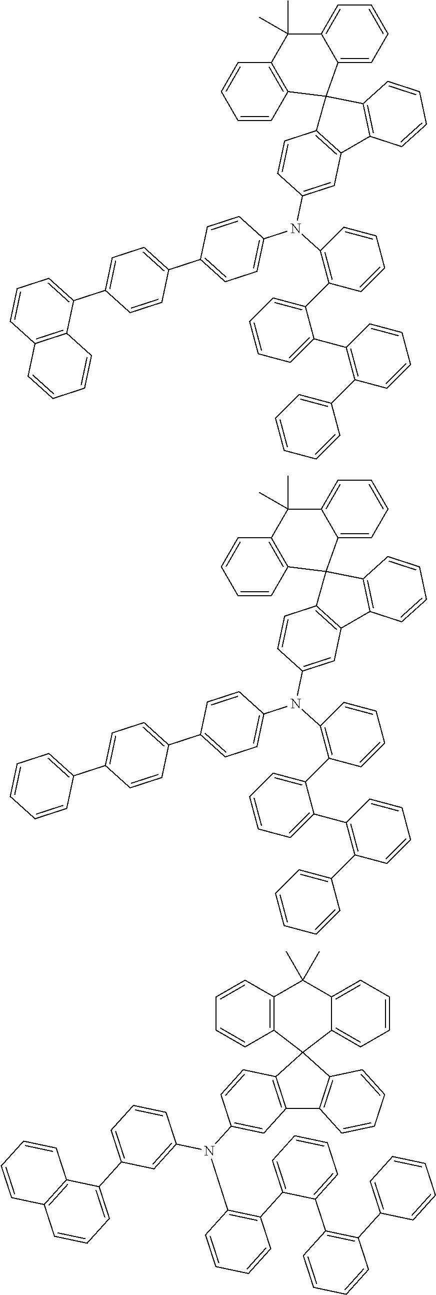

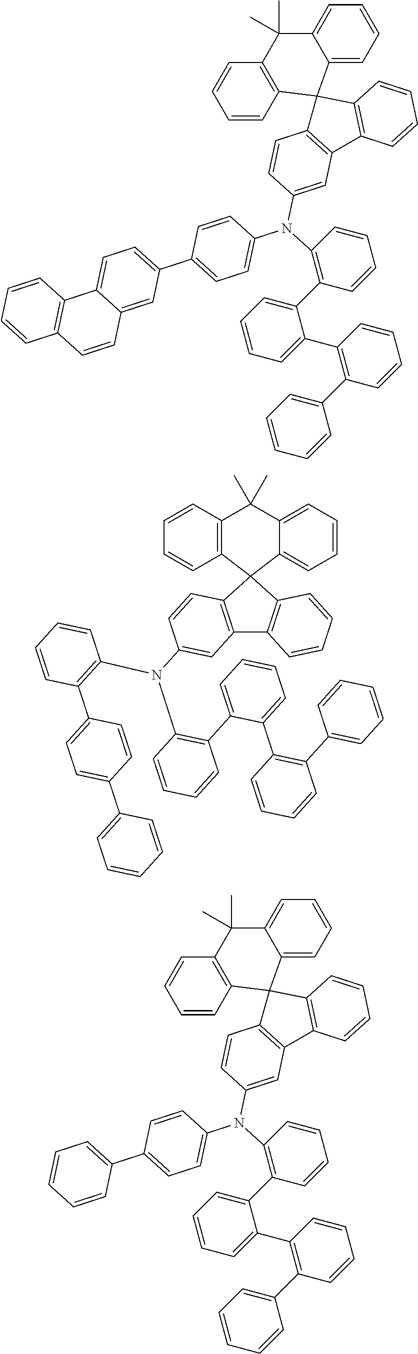

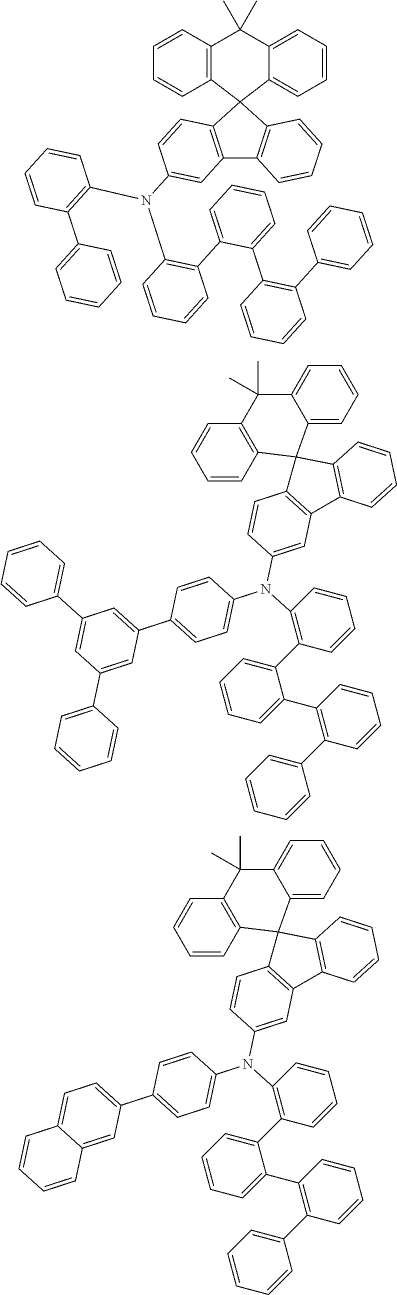

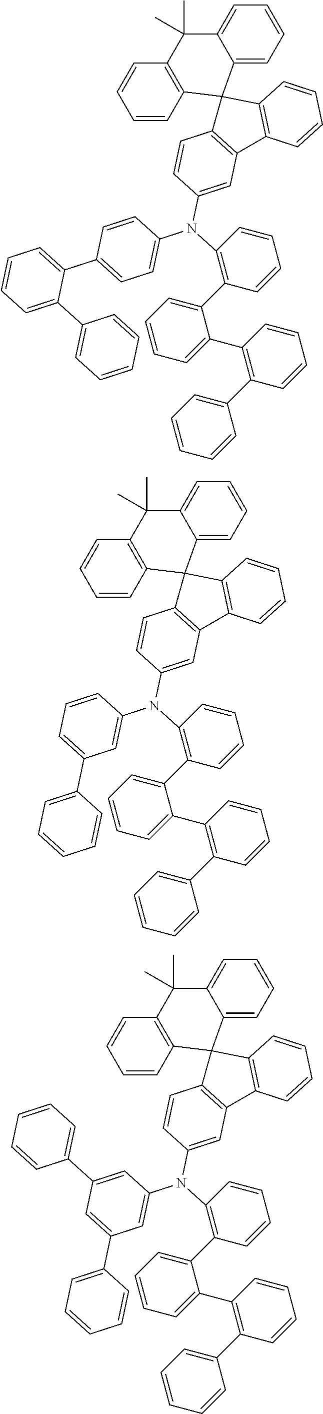

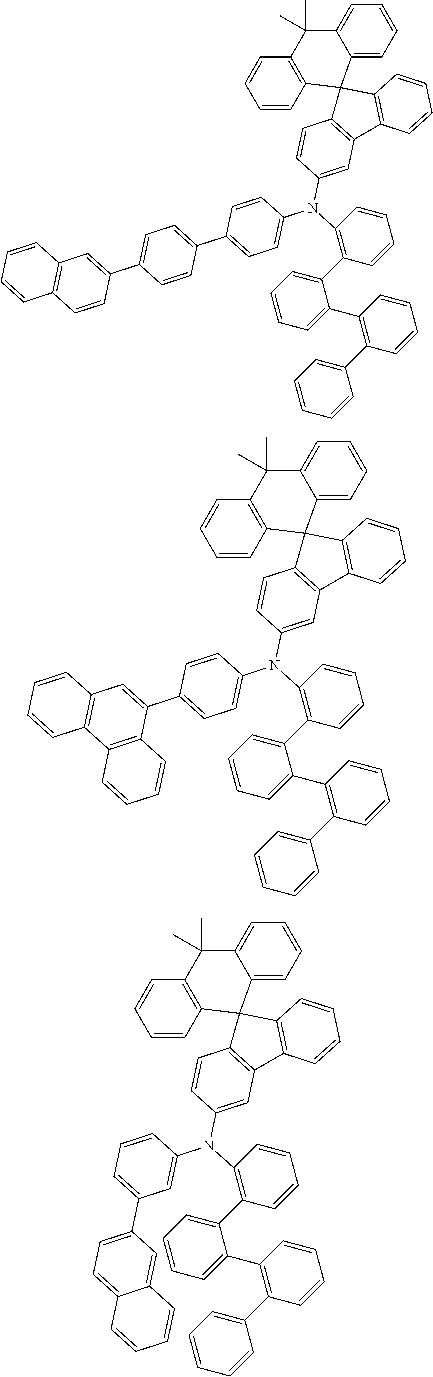

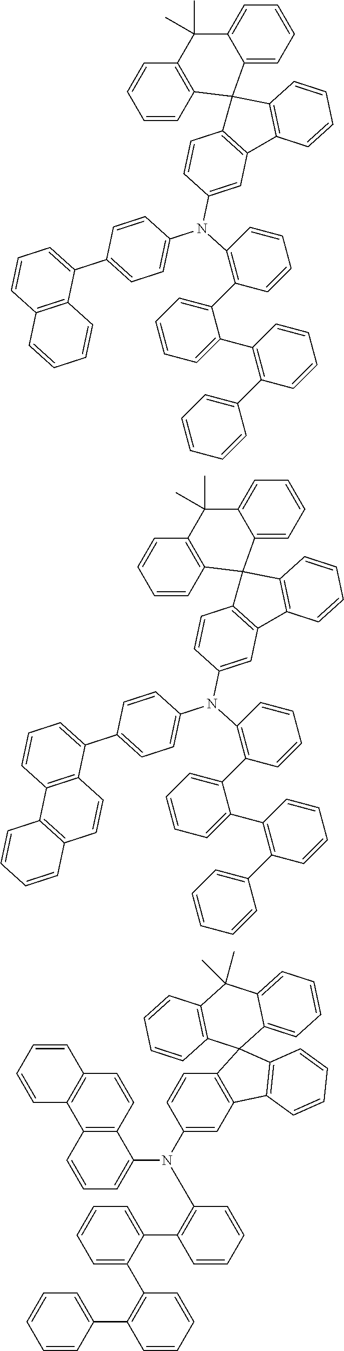

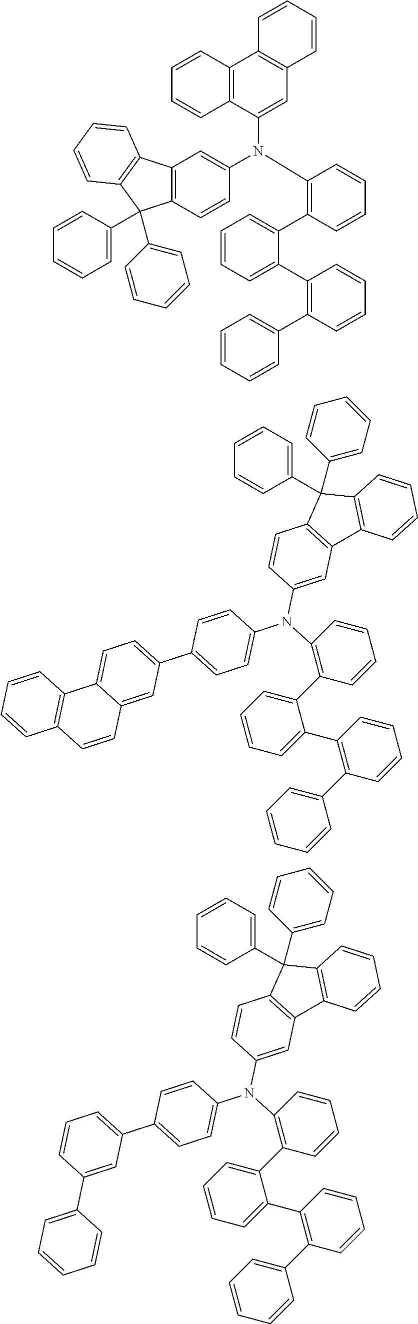

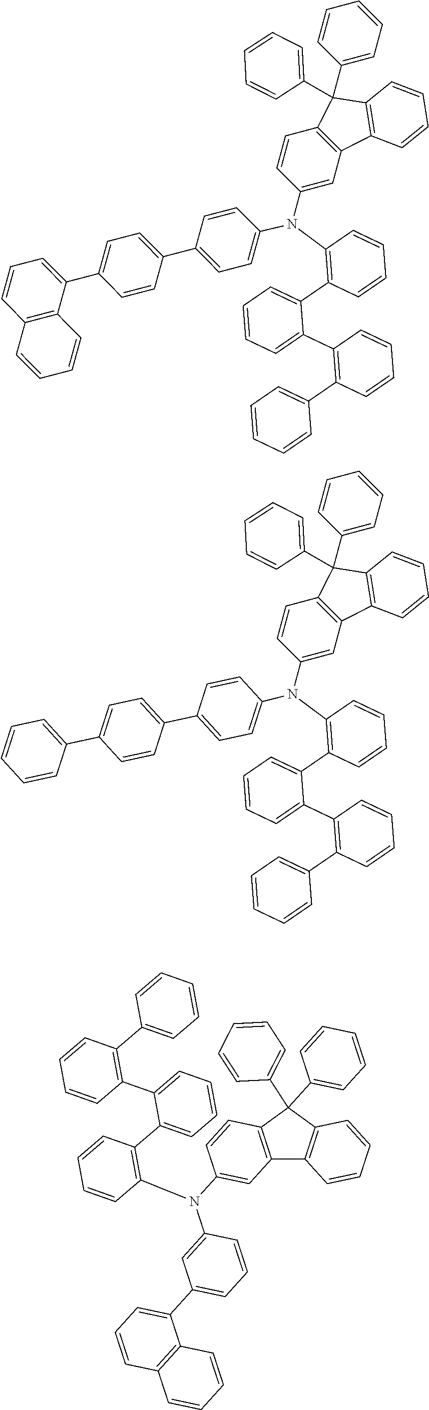 [SMILES: c1ccc(-c2ccccc2-c2ccccc2-c2ccccc2N(c2ccc(-c3ccc(-c4cccc5ccccc45)cc3)cc2)c2ccc3c(c2)-c2ccccc2C3(c2ccccc2)c2ccccc2)cc1 ; c1ccc(-c2ccc(-c3ccc(N(c4ccc5c(c4)-c4ccccc4C5(c4ccccc4)c4ccccc4)c4ccccc4-c4ccccc4-c4ccccc4-c4ccccc4)cc3)cc2)cc1 ; c1ccc(-c2ccccc2-c2ccccc2-c2ccccc2N(c2cccc(-c3cccc4ccccc34)c2)c2ccc3c(c2)-c2ccccc2C3(c2ccccc2)c2ccccc2)cc1]

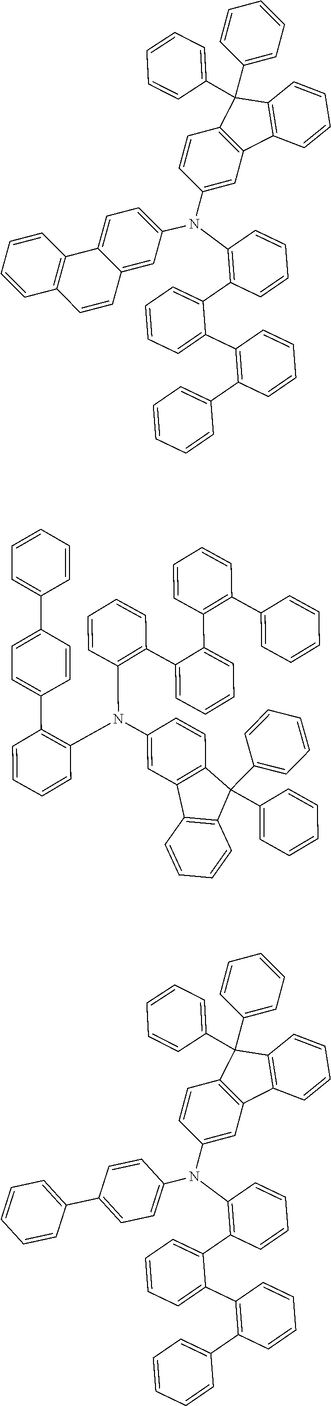 [SMILES: c1ccc(-c2ccccc2-c2ccccc2-c2ccccc2N(c2ccc3c(c2)-c2ccccc2C3(c2ccccc2)c2ccccc2)c2ccc3c(ccc4ccccc43)c2)cc1 ; c1ccc(-c2ccc(-c3ccccc3N(c3ccc4c(c3)-c3ccccc3C4(c3ccccc3)c3ccccc3)c3ccccc3-c3ccccc3-c3ccccc3-c3ccccc3)cc2)cc1 ; c1ccc(-c2ccc(N(c3ccc4c(c3)-c3ccccc3C4(c3ccccc3)c3ccccc3)c3ccccc3-c3ccccc3-c3ccccc3-c3ccccc3)cc2)cc1]

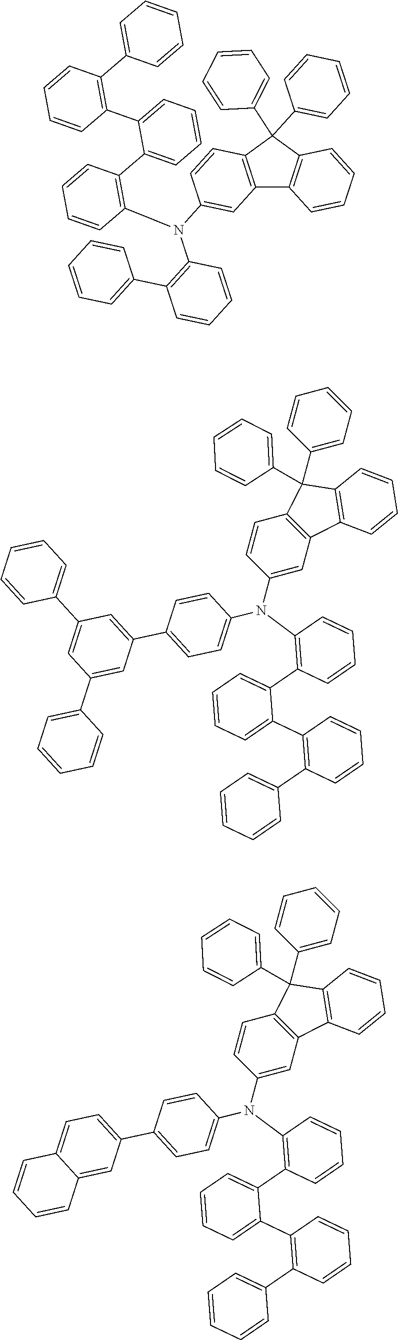 [SMILES: c1ccc(-c2ccccc2-c2ccccc2-c2ccccc2N(c2ccc3c(c2)-c2ccccc2C3(c2ccccc2)c2ccccc2)c2ccccc2-c2ccccc2)cc1 ; c1ccc(-c2cc(-c3ccccc3)cc(-c3ccc(N(c4ccc5c(c4)-c4ccccc4C5(c4ccccc4)c4ccccc4)c4ccccc4-c4ccccc4-c4ccccc4-c4ccccc4)cc3)c2)cc1 ; c1ccc(-c2ccccc2-c2ccccc2-c2ccccc2N(c2ccc(-c3ccc4ccccc4c3)cc2)c2ccc3c(c2)-c2ccccc2C3(c2ccccc2)c2ccccc2)cc1]

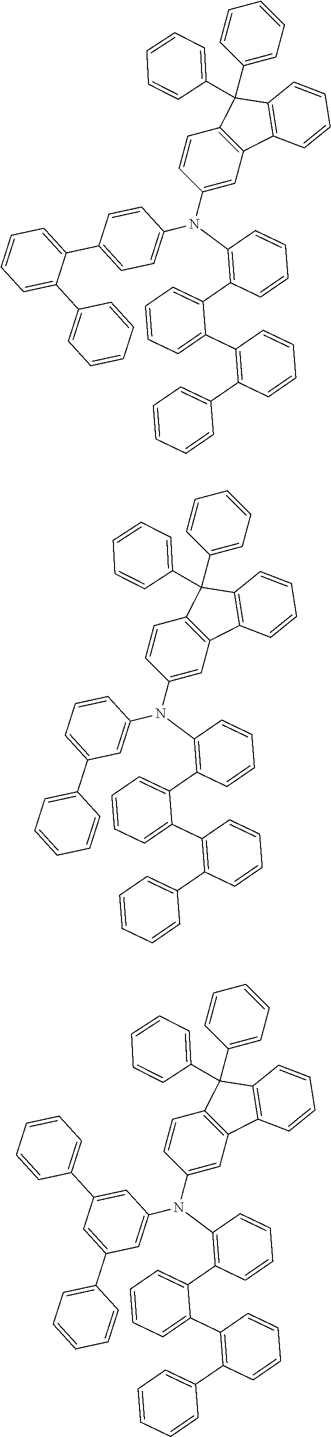 [SMILES: c1ccc(-c2ccccc2-c2ccc(N(c3ccc4c(c3)-c3ccccc3C4(c3ccccc3)c3ccccc3)c3ccccc3-c3ccccc3-c3ccccc3-c3ccccc3)cc2)cc1 ; c1ccc(-c2cccc(N(c3ccc4c(c3)-c3ccccc3C4(c3ccccc3)c3ccccc3)c3ccccc3-c3ccccc3-c3ccccc3-c3ccccc3)c2)cc1 ; c1ccc(-c2cc(-c3ccccc3)cc(N(c3ccc4c(c3)-c3ccccc3C4(c3ccccc3)c3ccccc3)c3ccccc3-c3ccccc3-c3ccccc3-c3ccccc3)c2)cc1]

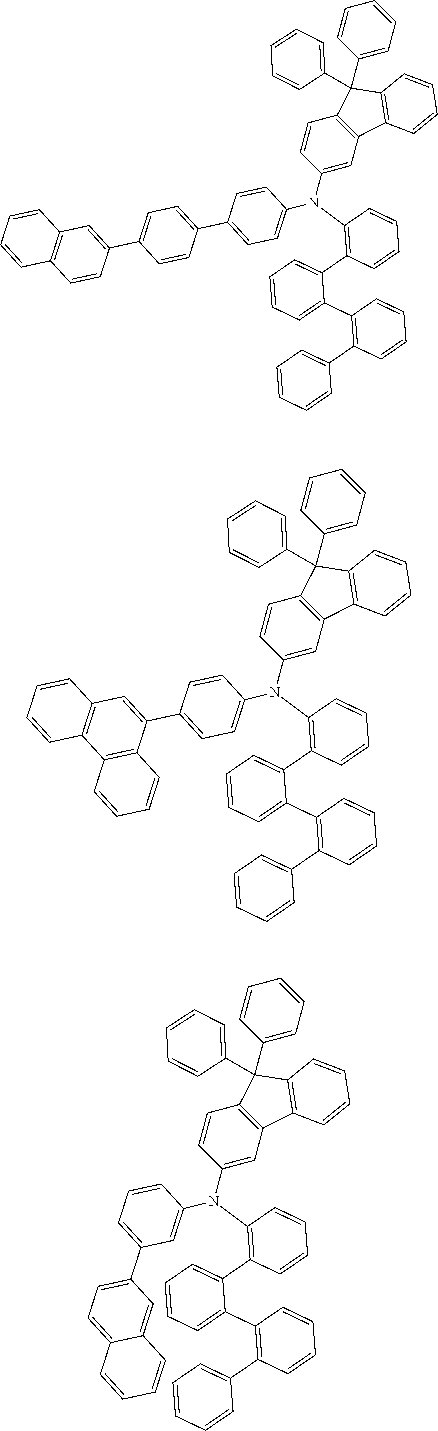 [SMILES: c1ccc(-c2ccccc2-c2ccccc2-c2ccccc2N(c2ccc(-c3ccc(-c4ccc5ccccc5c4)cc3)cc2)c2ccc3c(c2)-c2ccccc2C3(c2ccccc2)c2ccccc2)cc1 ; c1ccc(-c2ccccc2-c2ccccc2-c2ccccc2N(c2ccc(-c3cc4ccccc4c4ccccc34)cc2)c2ccc3c(c2)-c2ccccc2C3(c2ccccc2)c2ccccc2)cc1 ; c1ccc(-c2ccccc2-c2ccccc2-c2ccccc2N(c2cccc(-c3ccc4ccccc4c3)c2)c2ccc3c(c2)-c2ccccc2C3(c2ccccc2)c2ccccc2)cc1]

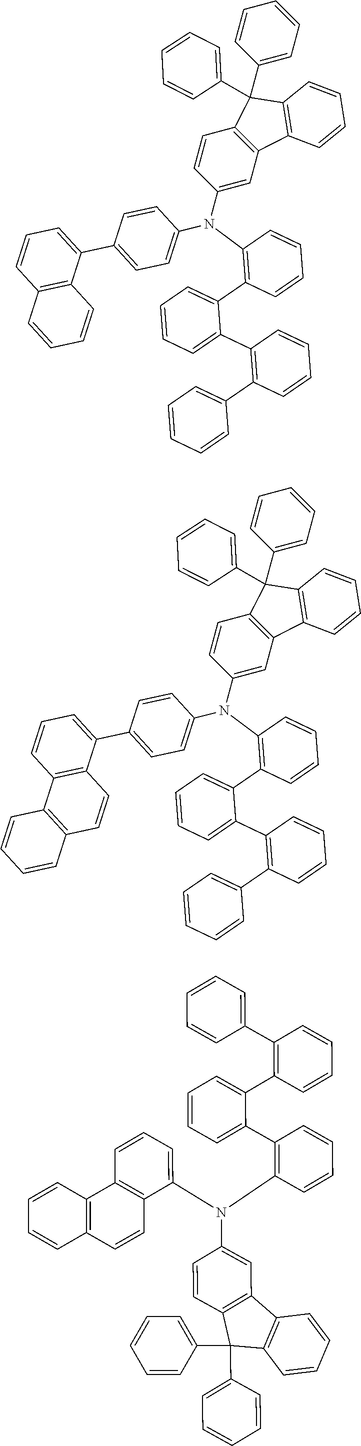

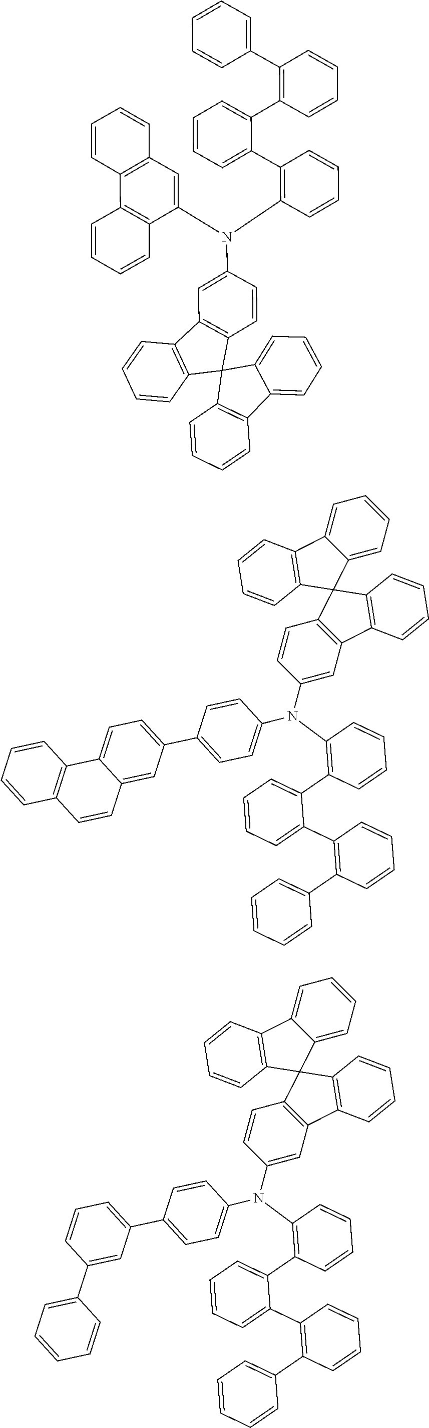

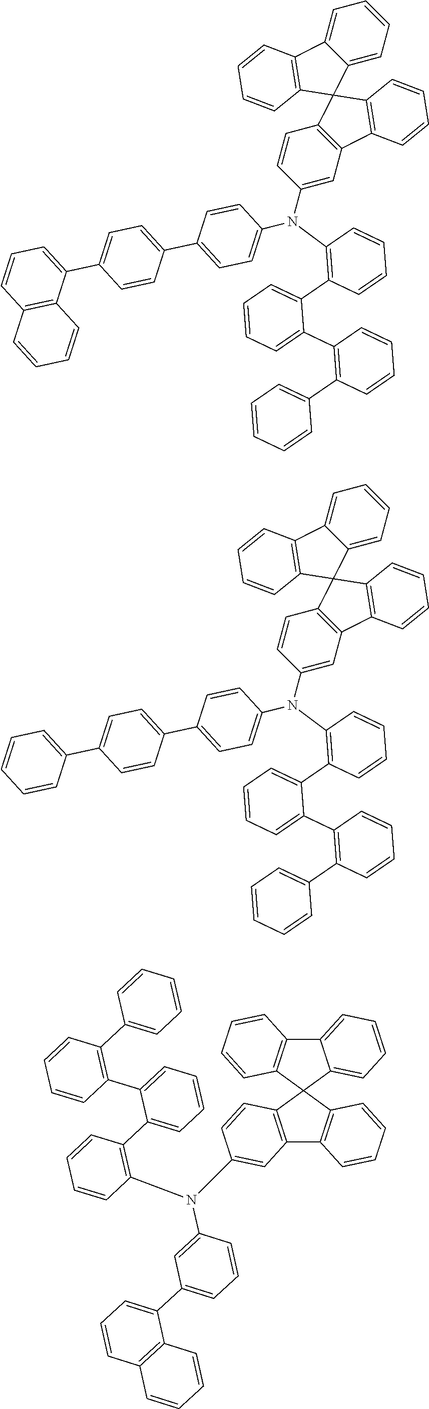 [SMILES: c1ccc(-c2ccccc2-c2ccccc2-c2ccccc2N(c2ccc(-c3ccc(-c4cccc5ccccc45)cc3)cc2)c2ccc3c(c2)-c2ccccc2C32c3ccccc3-c3ccccc32)cc1 ; c1ccc(-c2ccc(-c3ccc(N(c4ccc5c(c4)-c4ccccc4C54c5ccccc5-c5ccccc54)c4ccccc4-c4ccccc4-c4ccccc4-c4ccccc4)cc3)cc2)cc1 ; c1ccc(-c2ccccc2-c2ccccc2-c2ccccc2N(c2cccc(-c3cccc4ccccc34)c2)c2ccc3c(c2)-c2ccccc2C32c3ccccc3-c3ccccc32)cc1]

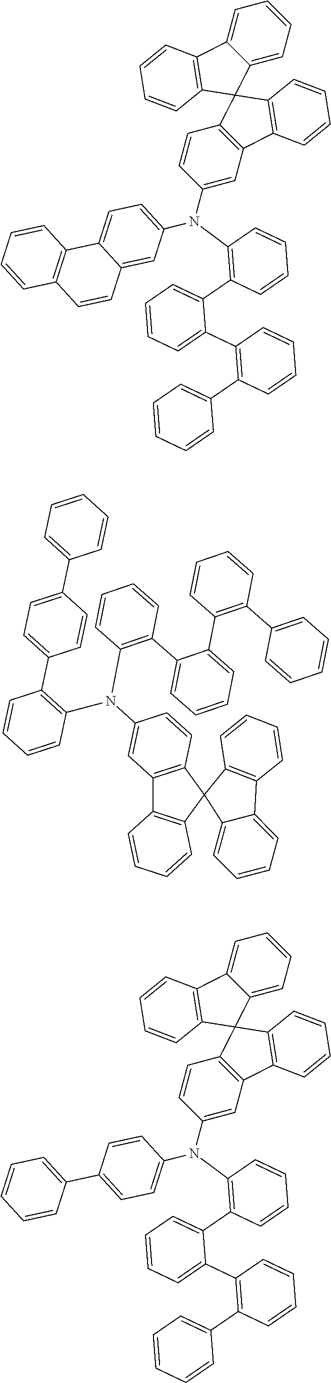 [SMILES: c1ccc(-c2ccccc2-c2ccccc2-c2ccccc2N(c2ccc3c(c2)-c2ccccc2C32c3ccccc3-c3ccccc32)c2ccc3c(ccc4ccccc43)c2)cc1 ; c1ccc(-c2ccc(-c3ccccc3N(c3ccc4c(c3)-c3ccccc3C43c4ccccc4-c4ccccc43)c3ccccc3-c3ccccc3-c3ccccc3-c3ccccc3)cc2)cc1 ; c1ccc(-c2ccc(N(c3ccc4c(c3)-c3ccccc3C43c4ccccc4-c4ccccc43)c3ccccc3-c3ccccc3-c3ccccc3-c3ccccc3)cc2)cc1]

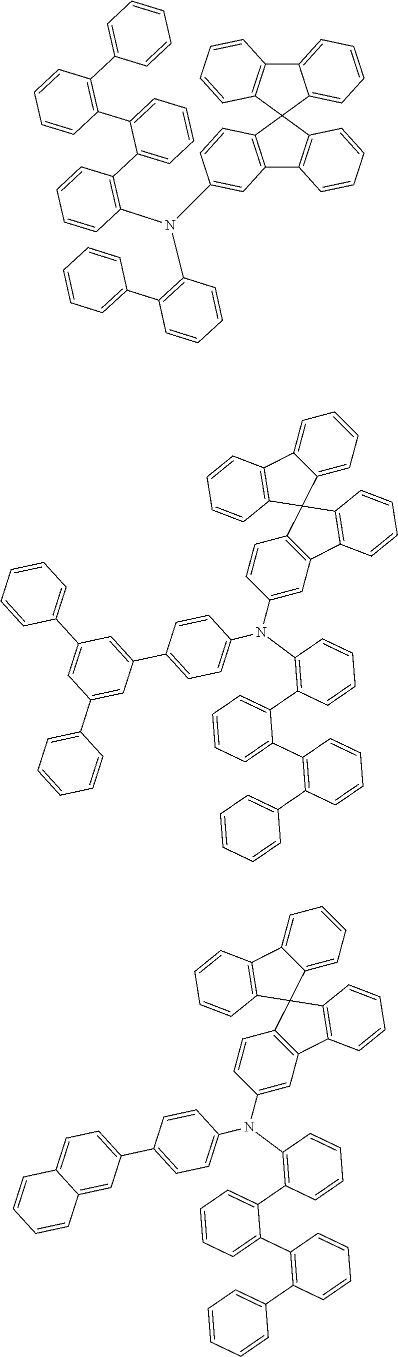 [SMILES: c1ccc(-c2ccccc2-c2ccccc2-c2ccccc2N(c2ccc3c(c2)-c2ccccc2C32c3ccccc3-c3ccccc32)c2ccccc2-c2ccccc2)cc1 ; c1ccc(-c2cc(-c3ccccc3)cc(-c3ccc(N(c4ccc5c(c4)-c4ccccc4C54c5ccccc5-c5ccccc54)c4ccccc4-c4ccccc4-c4ccccc4-c4ccccc4)cc3)c2)cc1 ; c1ccc(-c2ccccc2-c2ccccc2-c2ccccc2N(c2ccc(-c3ccc4ccccc4c3)cc2)c2ccc3c(c2)-c2ccccc2C32c3ccccc3-c3ccccc32)cc1]

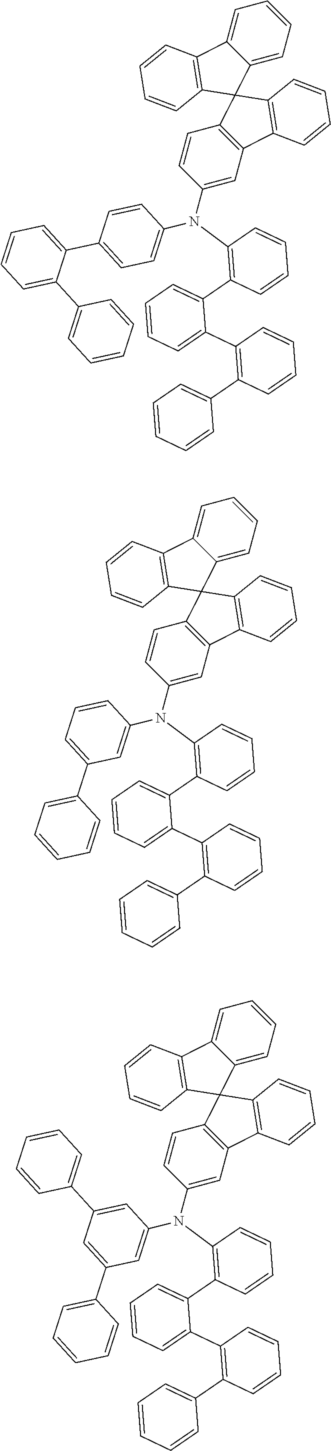 [SMILES: c1ccc(-c2ccccc2-c2ccc(N(c3ccc4c(c3)-c3ccccc3C43c4ccccc4-c4ccccc43)c3ccccc3-c3ccccc3-c3ccccc3-c3ccccc3)cc2)cc1 ; c1ccc(-c2cccc(N(c3ccc4c(c3)-c3ccccc3C43c4ccccc4-c4ccccc43)c3ccccc3-c3ccccc3-c3ccccc3-c3ccccc3)c2)cc1 ; c1ccc(-c2cc(-c3ccccc3)cc(N(c3ccc4c(c3)-c3ccccc3C43c4ccccc4-c4ccccc43)c3ccccc3-c3ccccc3-c3ccccc3-c3ccccc3)c2)cc1]

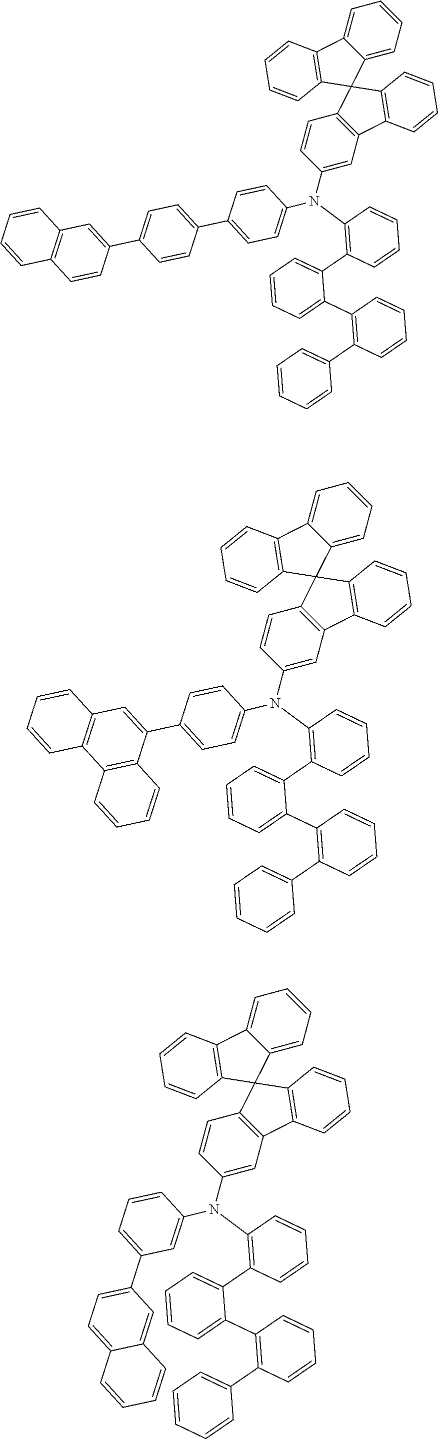 [SMILES: c1ccc(-c2ccccc2-c2ccccc2-c2ccccc2N(c2ccc(-c3ccc(-c4ccc5ccccc5c4)cc3)cc2)c2ccc3c(c2)-c2ccccc2C32c3ccccc3-c3ccccc32)cc1 ; c1ccc(-c2ccccc2-c2ccccc2-c2ccccc2N(c2ccc(-c3cc4ccccc4c4ccccc34)cc2)c2ccc3c(c2)-c2ccccc2C32c3ccccc3-c3ccccc32)cc1 ; c1ccc(-c2ccccc2-c2ccccc2-c2ccccc2N(c2cccc(-c3ccc4ccccc4c3)c2)c2ccc3c(c2)-c2ccccc2C32c3ccccc3-c3ccccc32)cc1]

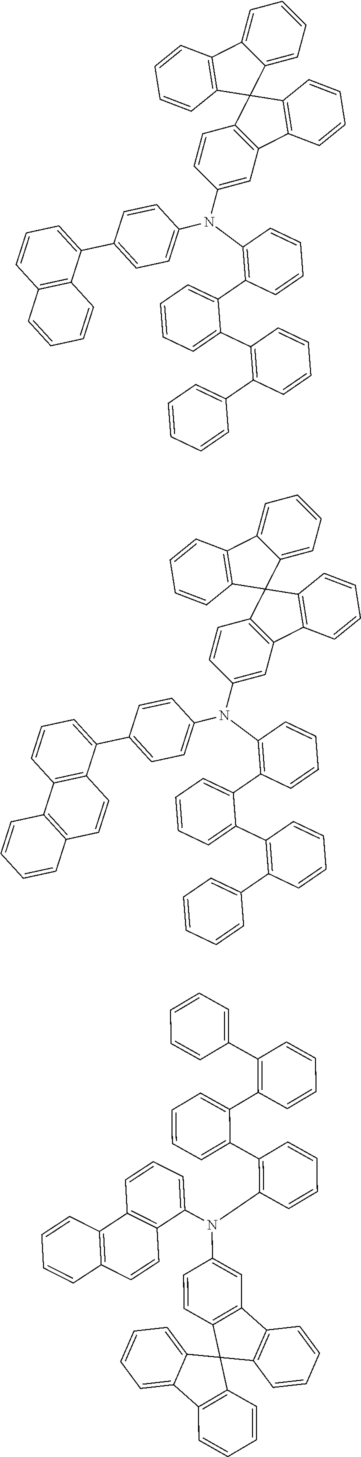 [SMILES: c1ccc(-c2ccccc2-c2ccccc2-c2ccccc2N(c2ccc(-c3cccc4ccccc34)cc2)c2ccc3c(c2)-c2ccccc2C32c3ccccc3-c3ccccc32)cc1 ; c1ccc(-c2ccccc2-c2ccccc2-c2ccccc2N(c2ccc(-c3cccc4c3ccc3ccccc34)cc2)c2ccc3c(c2)-c2ccccc2C32c3ccccc3-c3ccccc32)cc1 ; c1ccc(-c2ccccc2-c2ccccc2-c2ccccc2N(c2ccc3c(c2)-c2ccccc2C32c3ccccc3-c3ccccc32)c2cccc3c2ccc2ccccc23)cc1]

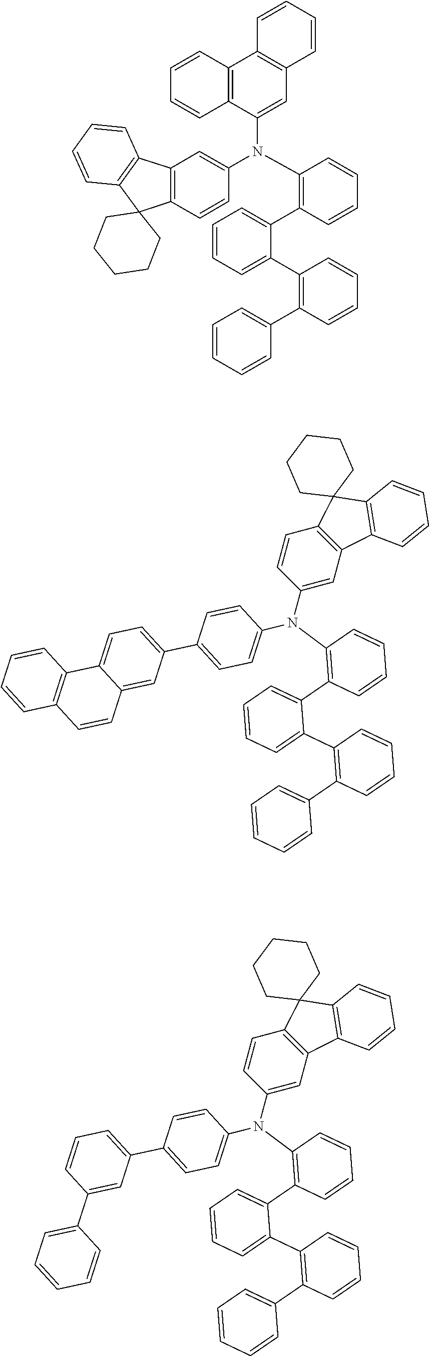 [SMILES: c1ccc(-c2ccccc2-c2ccccc2-c2ccccc2N(c2ccc3c(c2)-c2ccccc2C32CCCCC2)c2cc3ccccc3c3ccccc23)cc1 ; c1ccc(-c2ccccc2-c2ccccc2-c2ccccc2N(c2ccc(-c3ccc4c(ccc5ccccc54)c3)cc2)c2ccc3c(c2)-c2ccccc2C32CCCCC2)cc1 ; c1ccc(-c2cccc(-c3ccc(N(c4ccc5c(c4)-c4ccccc4C54CCCCC4)c4ccccc4-c4ccccc4-c4ccccc4-c4ccccc4)cc3)c2)cc1]

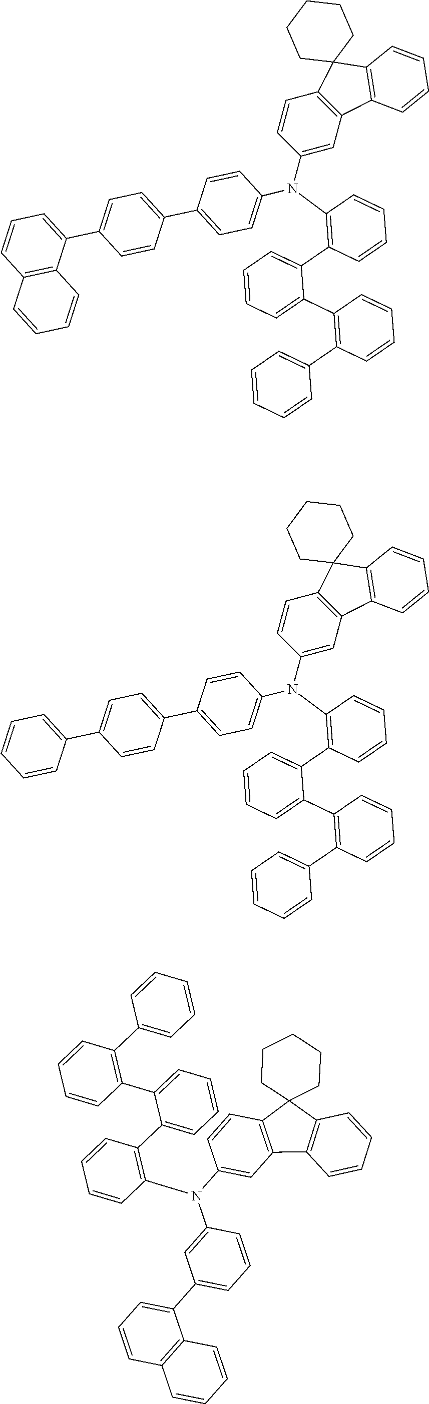 [SMILES: c1ccc(-c2ccccc2-c2ccccc2-c2ccccc2N(c2ccc(-c3ccc(-c4cccc5ccccc45)cc3)cc2)c2ccc3c(c2)-c2ccccc2C32CCCCC2)cc1 ; c1ccc(-c2ccc(-c3ccc(N(c4ccc5c(c4)-c4ccccc4C54CCCCC4)c4ccccc4-c4ccccc4-c4ccccc4-c4ccccc4)cc3)cc2)cc1 ; c1ccc(-c2ccccc2-c2ccccc2-c2ccccc2N(c2cccc(-c3cccc4ccccc34)c2)c2ccc3c(c2)-c2ccccc2C32CCCCC2)cc1]

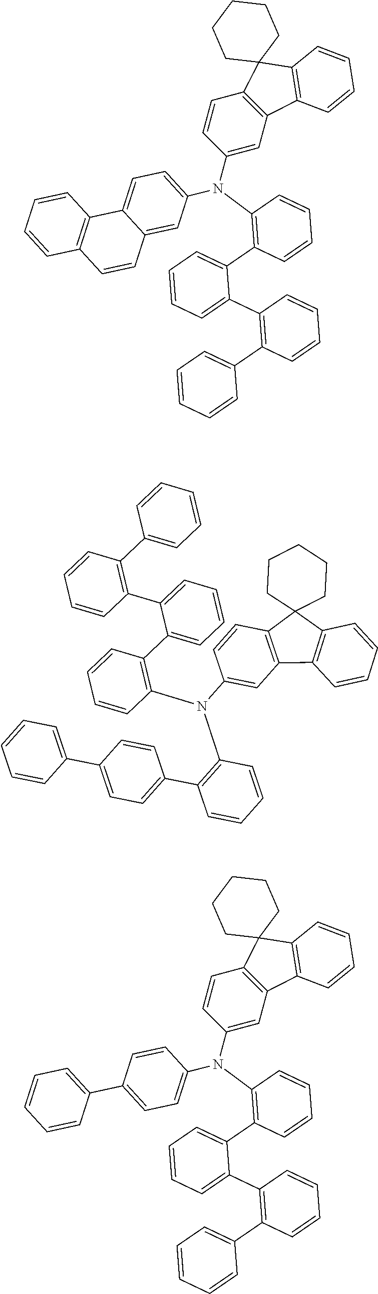 [SMILES: c1ccc(-c2ccccc2-c2ccccc2-c2ccccc2N(c2ccc3c(c2)-c2ccccc2C32CCCCC2)c2ccc3c(ccc4ccccc43)c2)cc1 ; c1ccc(-c2ccc(-c3ccccc3N(c3ccc4c(c3)-c3ccccc3C43CCCCC3)c3ccccc3-c3ccccc3-c3ccccc3-c3ccccc3)cc2)cc1 ; c1ccc(-c2ccc(N(c3ccc4c(c3)-c3ccccc3C43CCCCC3)c3ccccc3-c3ccccc3-c3ccccc3-c3ccccc3)cc2)cc1]

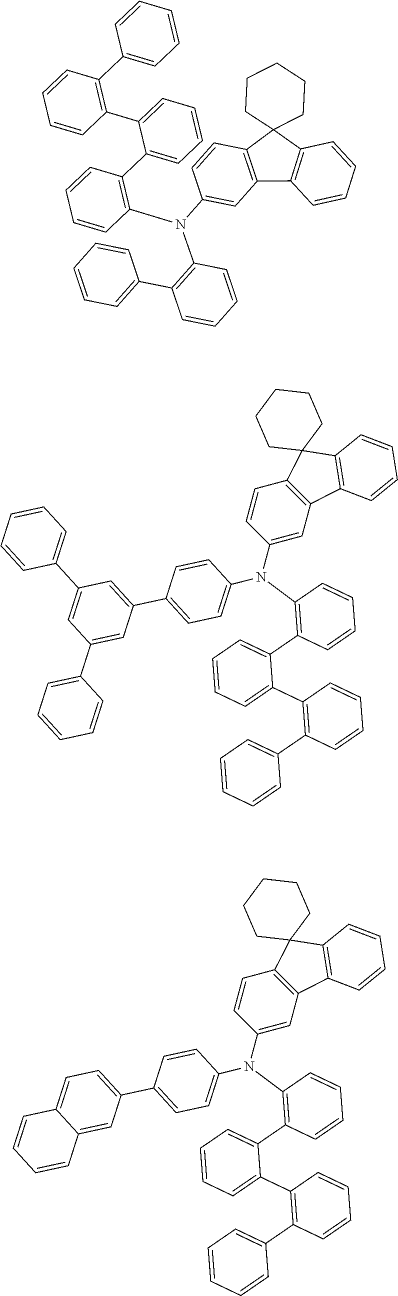 [SMILES: c1ccc(-c2ccccc2-c2ccccc2-c2ccccc2N(c2ccc3c(c2)-c2ccccc2C32CCCCC2)c2ccccc2-c2ccccc2)cc1 ; c1ccc(-c2cc(-c3ccccc3)cc(-c3ccc(N(c4ccc5c(c4)-c4ccccc4C54CCCCC4)c4ccccc4-c4ccccc4-c4ccccc4-c4ccccc4)cc3)c2)cc1 ; c1ccc(-c2ccccc2-c2ccccc2-c2ccccc2N(c2ccc(-c3ccc4ccccc4c3)cc2)c2ccc3c(c2)-c2ccccc2C32CCCCC2)cc1]

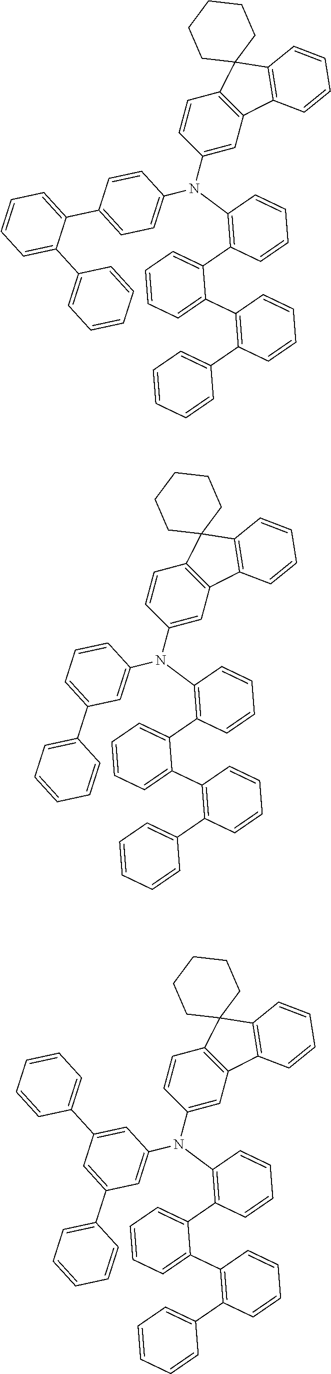 [SMILES: c1ccc(-c2ccccc2-c2ccc(N(c3ccc4c(c3)-c3ccccc3C43CCCCC3)c3ccccc3-c3ccccc3-c3ccccc3-c3ccccc3)cc2)cc1 ; c1ccc(-c2cccc(N(c3ccc4c(c3)-c3ccccc3C43CCCCC3)c3ccccc3-c3ccccc3-c3ccccc3-c3ccccc3)c2)cc1 ; c1ccc(-c2cc(-c3ccccc3)cc(N(c3ccc4c(c3)-c3ccccc3C43CCCCC3)c3ccccc3-c3ccccc3-c3ccccc3-c3ccccc3)c2)cc1]

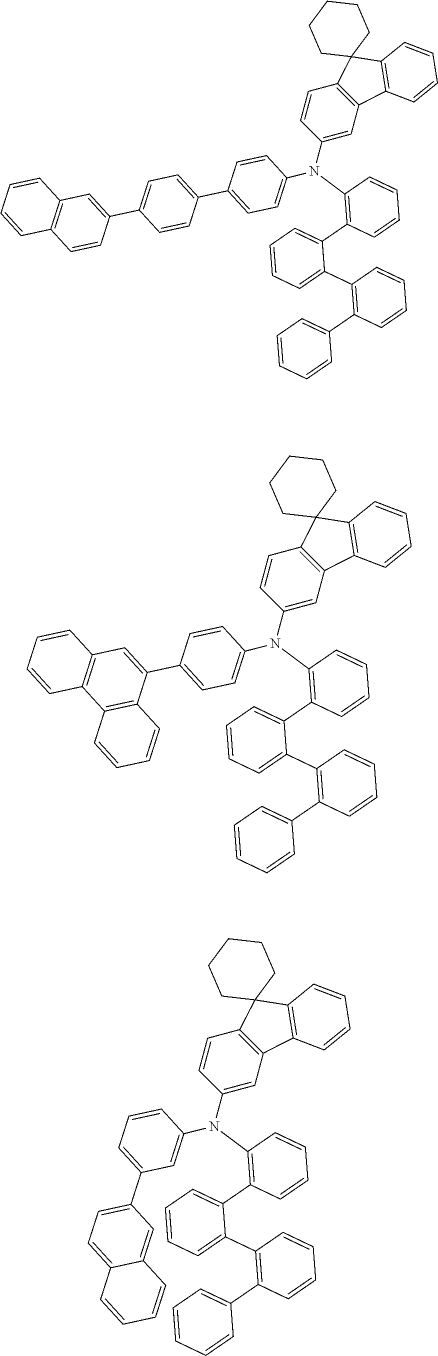 [SMILES: c1ccc(-c2ccccc2-c2ccccc2-c2ccccc2N(c2ccc(-c3ccc(-c4ccc5ccccc5c4)cc3)cc2)c2ccc3c(c2)-c2ccccc2C32CCCCC2)cc1 ; c1ccc(-c2ccccc2-c2ccccc2-c2ccccc2N(c2ccc(-c3cc4ccccc4c4ccccc34)cc2)c2ccc3c(c2)-c2ccccc2C32CCCCC2)cc1 ; c1ccc(-c2ccccc2-c2ccccc2-c2ccccc2N(c2cccc(-c3ccc4ccccc4c3)c2)c2ccc3c(c2)-c2ccccc2C32CCCCC2)cc1]

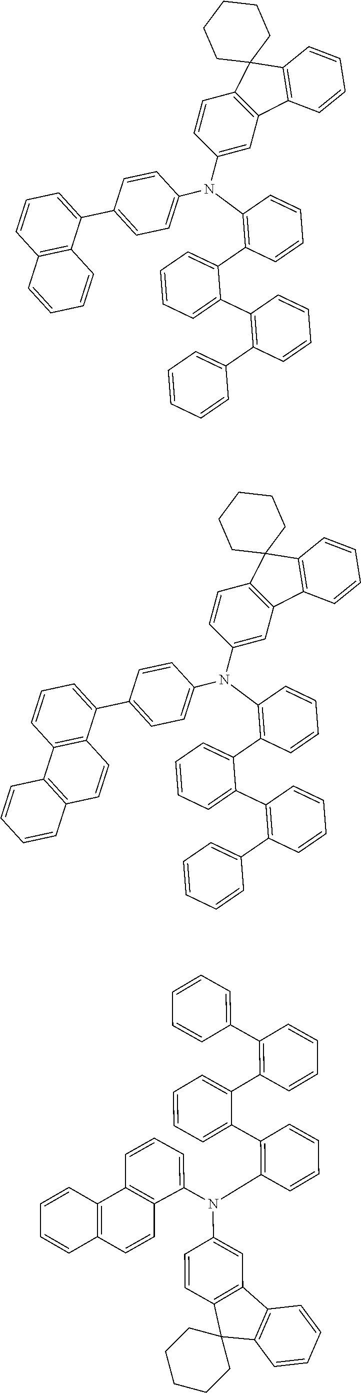 [SMILES: c1ccc(-c2ccccc2-c2ccccc2-c2ccccc2N(c2ccc(-c3cccc4ccccc34)cc2)c2ccc3c(c2)-c2ccccc2C32CCCCC2)cc1 ; c1ccc(-c2ccccc2-c2ccccc2-c2ccccc2N(c2ccc(-c3cccc4c3ccc3ccccc34)cc2)c2ccc3c(c2)-c2ccccc2C32CCCCC2)cc1 ; c1ccc(-c2ccccc2-c2ccccc2-c2ccccc2N(c2ccc3c(c2)-c2ccccc2C32CCCCC2)c2cccc3c2ccc2ccccc23)cc1]

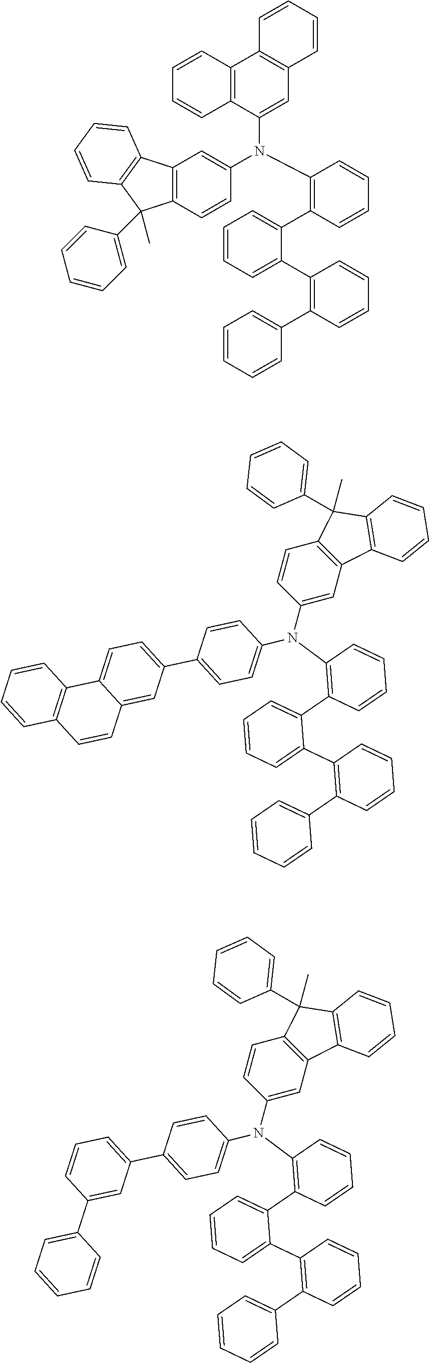 [SMILES: CC1(c2ccccc2)c2ccccc2-c2cc(N(c3ccccc3-c3ccccc3-c3ccccc3-c3ccccc3)c3cc4ccccc4c4ccccc34)ccc21 ; CC1(c2ccccc2)c2ccccc2-c2cc(N(c3ccc(-c4ccc5c(ccc6ccccc65)c4)cc3)c3ccccc3-c3ccccc3-c3ccccc3-c3ccccc3)ccc21 ; CC1(c2ccccc2)c2ccccc2-c2cc(N(c3ccc(-c4cccc(-c5ccccc5)c4)cc3)c3ccccc3-c3ccccc3-c3ccccc3-c3ccccc3)ccc21]

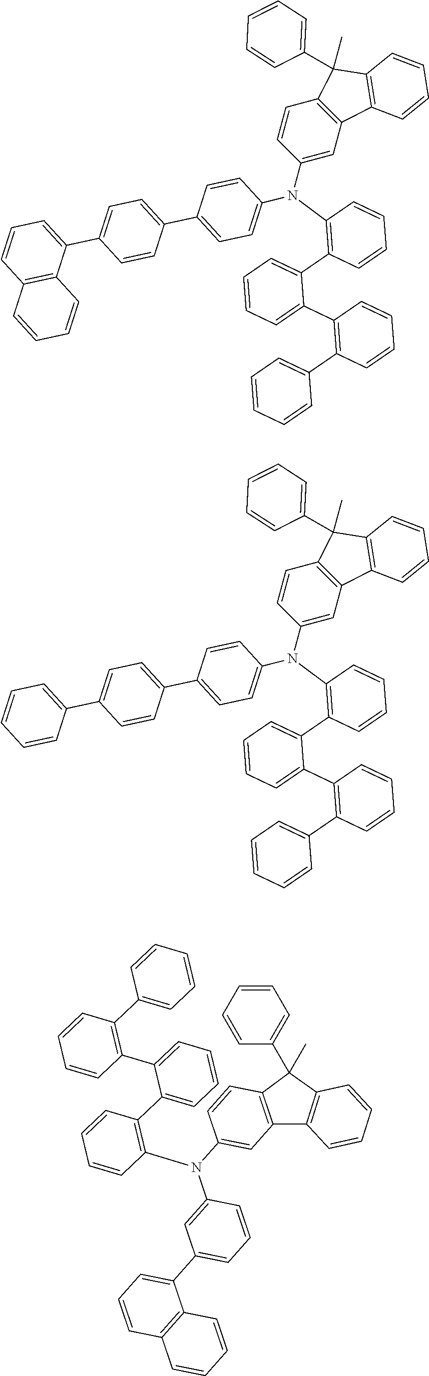

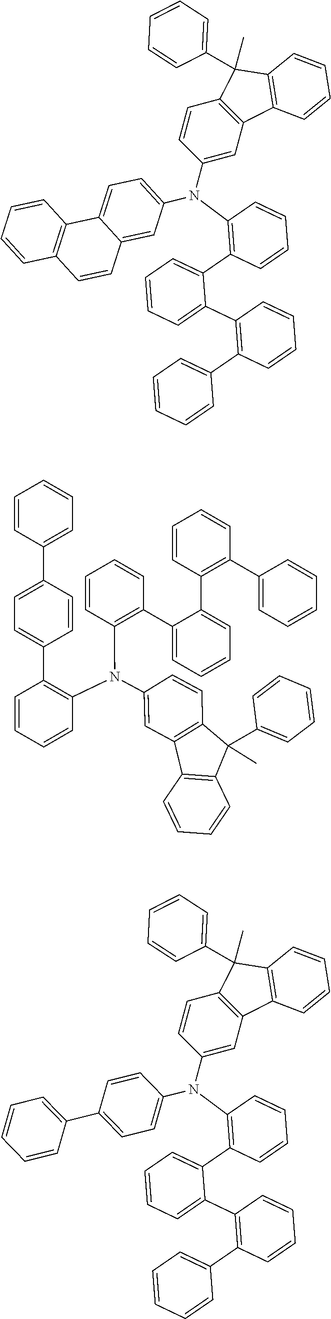 [SMILES: CC1(c2ccccc2)c2ccccc2-c2cc(N(c3ccc4c(ccc5ccccc54)c3)c3ccccc3-c3ccccc3-c3ccccc3-c3ccccc3)ccc21 ; CC1(c2ccccc2)c2ccccc2-c2cc(N(c3ccccc3-c3ccc(-c4ccccc4)cc3)c3ccccc3-c3ccccc3-c3ccccc3-c3ccccc3)ccc21 ; CC1(c2ccccc2)c2ccccc2-c2cc(N(c3ccc(-c4ccccc4)cc3)c3ccccc3-c3ccccc3-c3ccccc3-c3ccccc3)ccc21]

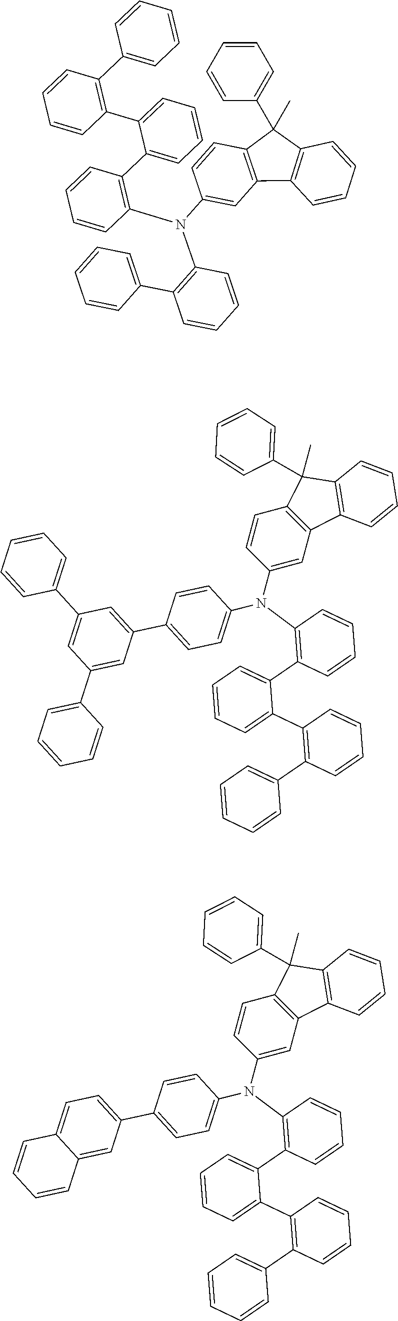 [SMILES: CC1(c2ccccc2)c2ccccc2-c2cc(N(c3ccccc3-c3ccccc3)c3ccccc3-c3ccccc3-c3ccccc3-c3ccccc3)ccc21 ; CC1(c2ccccc2)c2ccccc2-c2cc(N(c3ccc(-c4cc(-c5ccccc5)cc(-c5ccccc5)c4)cc3)c3ccccc3-c3ccccc3-c3ccccc3-c3ccccc3)ccc21 ; CC1(c2ccccc2)c2ccccc2-c2cc(N(c3ccc(-c4ccc5ccccc5c4)cc3)c3ccccc3-c3ccccc3-c3ccccc3-c3ccccc3)ccc21]

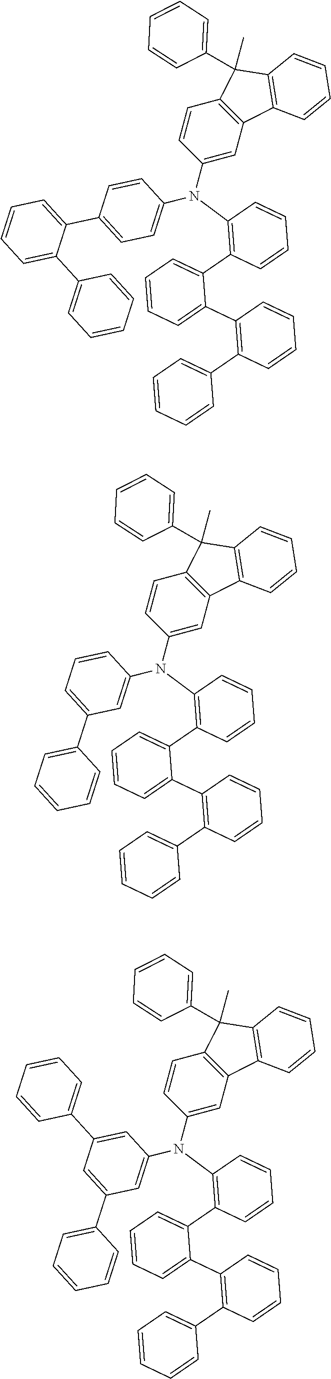 [SMILES: CC1(c2ccccc2)c2ccccc2-c2cc(N(c3ccc(-c4ccccc4-c4ccccc4)cc3)c3ccccc3-c3ccccc3-c3ccccc3-c3ccccc3)ccc21 ; CC1(c2ccccc2)c2ccccc2-c2cc(N(c3cccc(-c4ccccc4)c3)c3ccccc3-c3ccccc3-c3ccccc3-c3ccccc3)ccc21 ; CC1(c2ccccc2)c2ccccc2-c2cc(N(c3cc(-c4ccccc4)cc(-c4ccccc4)c3)c3ccccc3-c3ccccc3-c3ccccc3-c3ccccc3)ccc21]

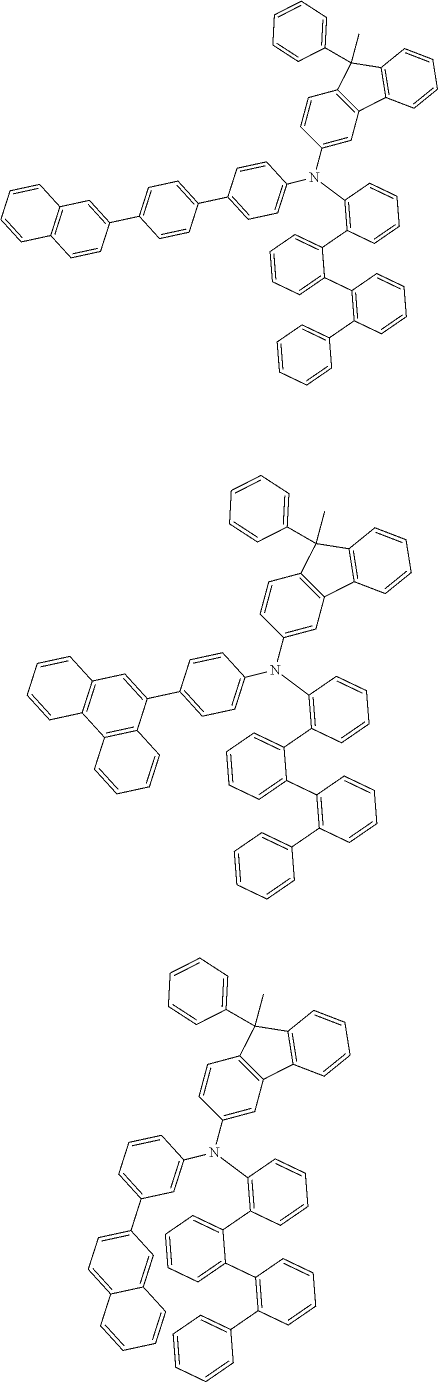 [SMILES: CC1(c2ccccc2)c2ccccc2-c2cc(N(c3ccc(-c4ccc(-c5ccc6ccccc6c5)cc4)cc3)c3ccccc3-c3ccccc3-c3ccccc3-c3ccccc3)ccc21 ; CC1(c2ccccc2)c2ccccc2-c2cc(N(c3ccc(-c4cc5ccccc5c5ccccc45)cc3)c3ccccc3-c3ccccc3-c3ccccc3-c3ccccc3)ccc21 ; CC1(c2ccccc2)c2ccccc2-c2cc(N(c3cccc(-c4ccc5ccccc5c4)c3)c3ccccc3-c3ccccc3-c3ccccc3-c3ccccc3)ccc21]

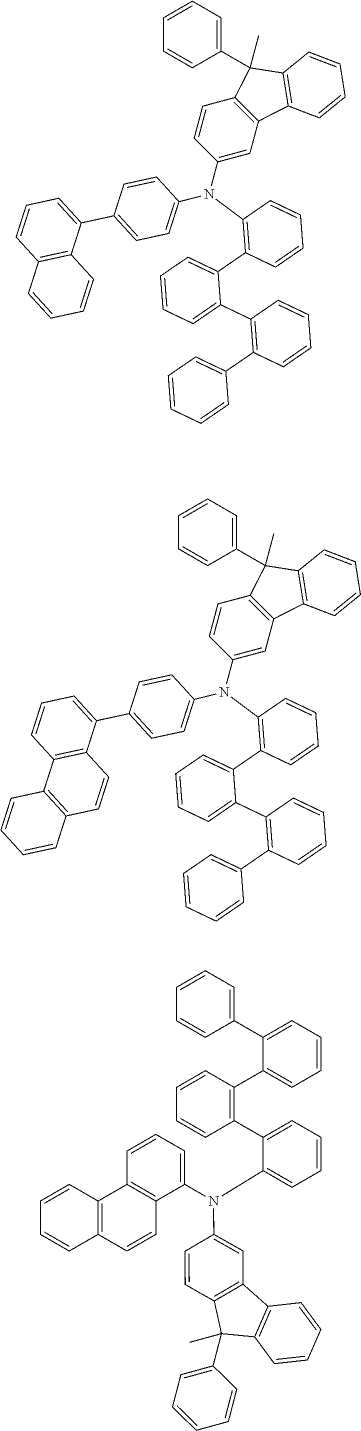 [SMILES: CC1(c2ccccc2)c2ccccc2-c2cc(N(c3ccc(-c4cccc5ccccc45)cc3)c3ccccc3-c3ccccc3-c3ccccc3-c3ccccc3)ccc21 ; CC1(c2ccccc2)c2ccccc2-c2cc(N(c3ccc(-c4cccc5c4ccc4ccccc45)cc3)c3ccccc3-c3ccccc3-c3ccccc3-c3ccccc3)ccc21 ; CC1(c2ccccc2)c2ccccc2-c2cc(N(c3ccccc3-c3ccccc3-c3ccccc3-c3ccccc3)c3cccc4c3ccc3ccccc34)ccc21]

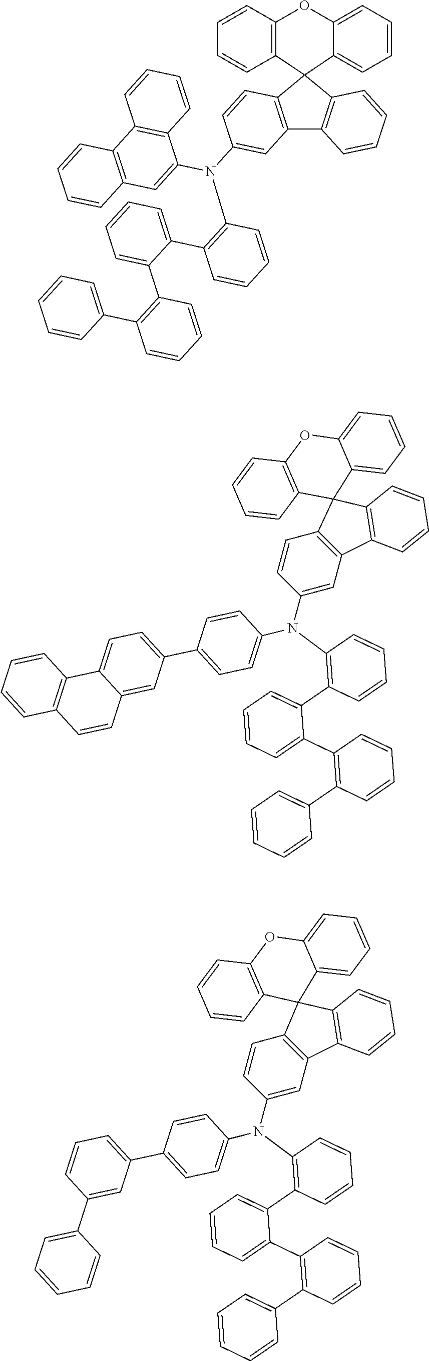 [SMILES: c1ccc(-c2ccccc2-c2ccccc2-c2ccccc2N(c2ccc3c(c2)-c2ccccc2C32c3ccccc3Oc3ccccc32)c2cc3ccccc3c3ccccc23)cc1 ; c1ccc(-c2ccccc2-c2ccccc2-c2ccccc2N(c2ccc(-c3ccc4c(ccc5ccccc54)c3)cc2)c2ccc3c(c2)-c2ccccc2C32c3ccccc3Oc3ccccc32)cc1 ; c1ccc(-c2cccc(-c3ccc(N(c4ccc5c(c4)-c4ccccc4C54c5ccccc5Oc5ccccc54)c4ccccc4-c4ccccc4-c4ccccc4-c4ccccc4)cc3)c2)cc1]

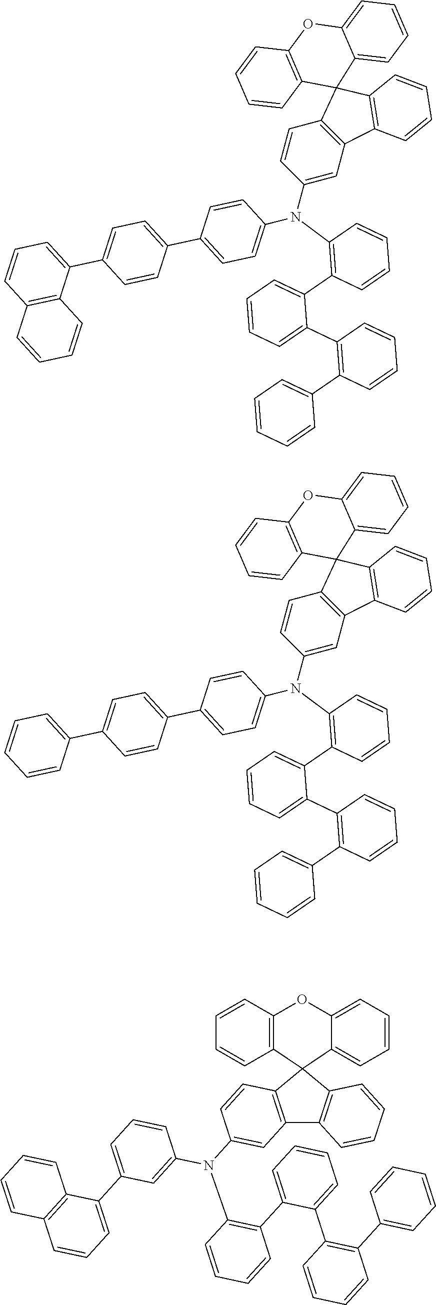 [SMILES: c1ccc(-c2ccccc2-c2ccccc2-c2ccccc2N(c2ccc(-c3ccc(-c4cccc5ccccc45)cc3)cc2)c2ccc3c(c2)-c2ccccc2C32c3ccccc3Oc3ccccc32)cc1 ; c1ccc(-c2ccc(-c3ccc(N(c4ccc5c(c4)-c4ccccc4C54c5ccccc5Oc5ccccc54)c4ccccc4-c4ccccc4-c4ccccc4-c4ccccc4)cc3)cc2)cc1 ; c1ccc(-c2ccccc2-c2ccccc2-c2ccccc2N(c2cccc(-c3cccc4ccccc34)c2)c2ccc3c(c2)-c2ccccc2C32c3ccccc3Oc3ccccc32)cc1]

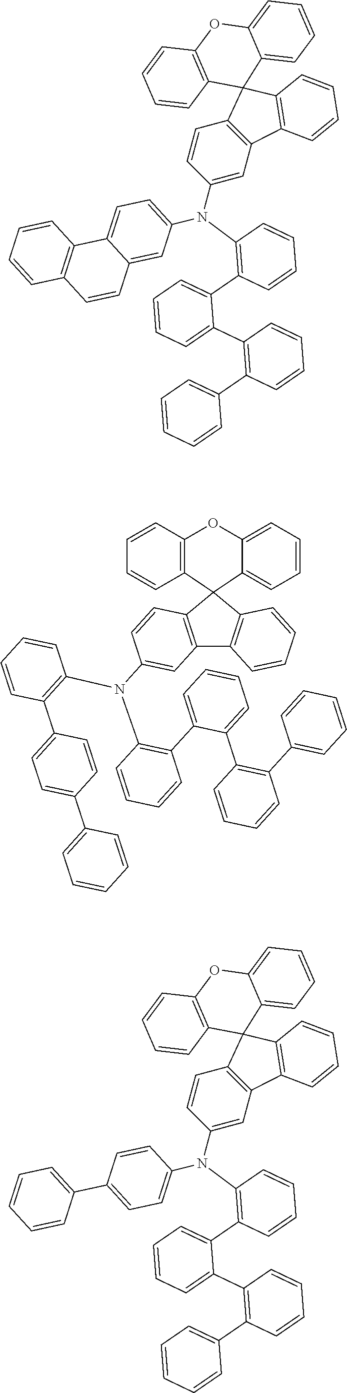 [SMILES: c1ccc(-c2ccccc2-c2ccccc2-c2ccccc2N(c2ccc3c(c2)-c2ccccc2C32c3ccccc3Oc3ccccc32)c2ccc3c(ccc4ccccc43)c2)cc1 ; c1ccc(-c2ccc(-c3ccccc3N(c3ccc4c(c3)-c3ccccc3C43c4ccccc4Oc4ccccc43)c3ccccc3-c3ccccc3-c3ccccc3-c3ccccc3)cc2)cc1 ; c1ccc(-c2ccc(N(c3ccc4c(c3)-c3ccccc3C43c4ccccc4Oc4ccccc43)c3ccccc3-c3ccccc3-c3ccccc3-c3ccccc3)cc2)cc1]

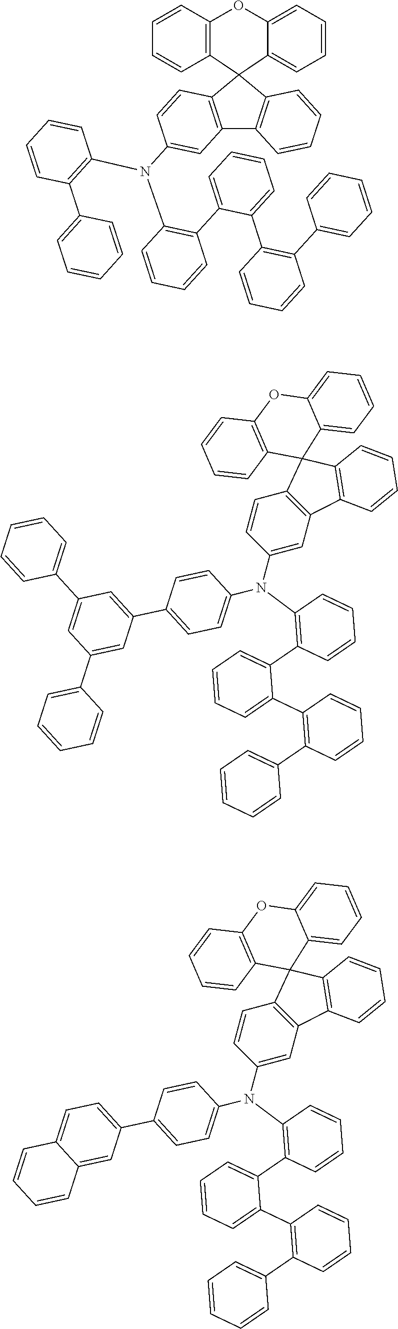 [SMILES: c1ccc(-c2ccccc2-c2ccccc2-c2ccccc2N(c2ccc3c(c2)-c2ccccc2C32c3ccccc3Oc3ccccc32)c2ccccc2-c2ccccc2)cc1 ; c1ccc(-c2cc(-c3ccccc3)cc(-c3ccc(N(c4ccc5c(c4)-c4ccccc4C54c5ccccc5Oc5ccccc54)c4ccccc4-c4ccccc4-c4ccccc4-c4ccccc4)cc3)c2)cc1 ; c1ccc(-c2ccccc2-c2ccccc2-c2ccccc2N(c2ccc(-c3ccc4ccccc4c3)cc2)c2ccc3c(c2)-c2ccccc2C32c3ccccc3Oc3ccccc32)cc1]

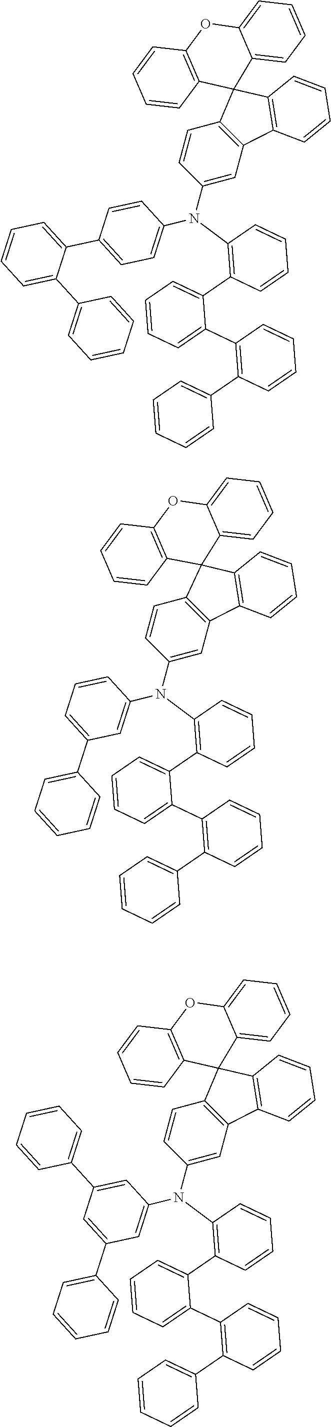 [SMILES: c1ccc(-c2ccccc2-c2ccc(N(c3ccc4c(c3)-c3ccccc3C43c4ccccc4Oc4ccccc43)c3ccccc3-c3ccccc3-c3ccccc3-c3ccccc3)cc2)cc1 ; c1ccc(-c2cccc(N(c3ccc4c(c3)-c3ccccc3C43c4ccccc4Oc4ccccc43)c3ccccc3-c3ccccc3-c3ccccc3-c3ccccc3)c2)cc1 ; c1ccc(-c2cc(-c3ccccc3)cc(N(c3ccc4c(c3)-c3ccccc3C43c4ccccc4Oc4ccccc43)c3ccccc3-c3ccccc3-c3ccccc3-c3ccccc3)c2)cc1]

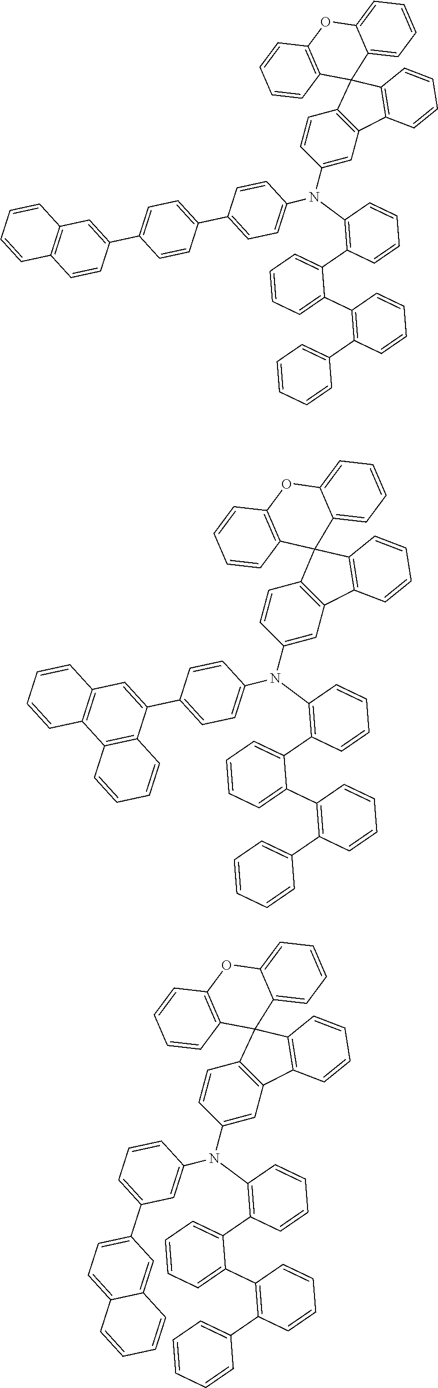

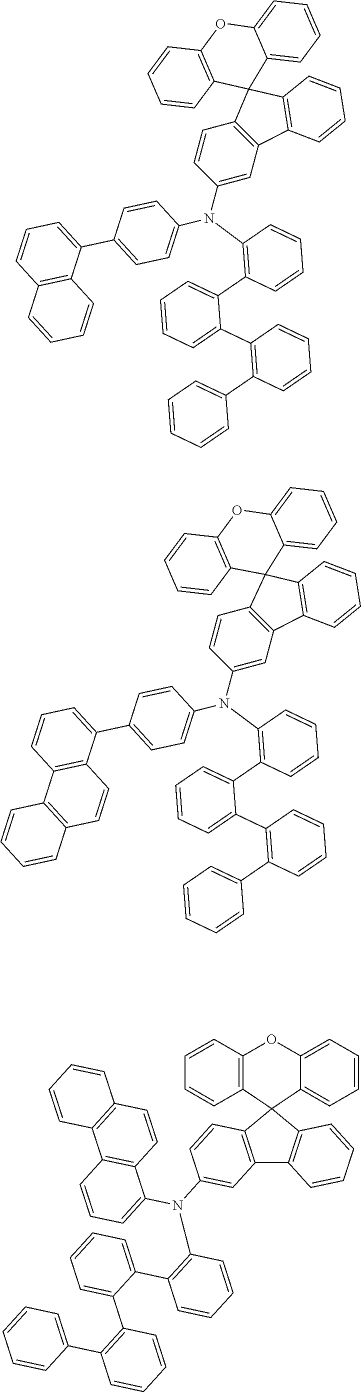 [SMILES: c1ccc(-c2ccccc2-c2ccccc2-c2ccccc2N(c2ccc(-c3cccc4ccccc34)cc2)c2ccc3c(c2)-c2ccccc2C32c3ccccc3Oc3ccccc32)cc1 ; c1ccc(-c2ccccc2-c2ccccc2-c2ccccc2N(c2ccc(-c3cccc4c3ccc3ccccc34)cc2)c2ccc3c(c2)-c2ccccc2C32c3ccccc3Oc3ccccc32)cc1 ; c1ccc(-c2ccccc2-c2ccccc2-c2ccccc2N(c2ccc3c(c2)-c2ccccc2C32c3ccccc3Oc3ccccc32)c2cccc3c2ccc2ccccc23)cc1]

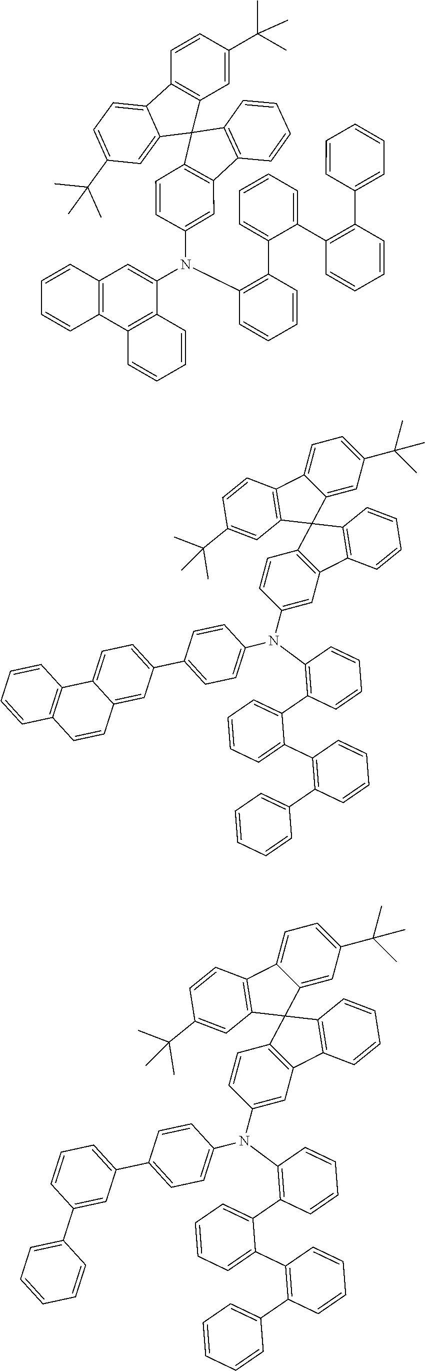 [SMILES: CC(C)(C)c1ccc2c(c1)C1(c3ccccc3-c3cc(N(c4ccccc4-c4ccccc4-c4ccccc4-c4ccccc4)c4cc5ccccc5c5ccccc45)ccc31)c1cc(C(C)(C)C)ccc1-2 ; CC(C)(C)c1ccc2c(c1)C1(c3ccccc3-c3cc(N(c4ccc(-c5ccc6c(ccc7ccccc76)c5)cc4)c4ccccc4-c4ccccc4-c4ccccc4-c4ccccc4)ccc31)c1cc(C(C)(C)C)ccc1-2 ; CC(C)(C)c1ccc2c(c1)C1(c3ccccc3-c3cc(N(c4ccc(-c5cccc(-c6ccccc6)c5)cc4)c4ccccc4-c4ccccc4-c4ccccc4-c4ccccc4)ccc31)c1cc(C(C)(C)C)ccc1-2]

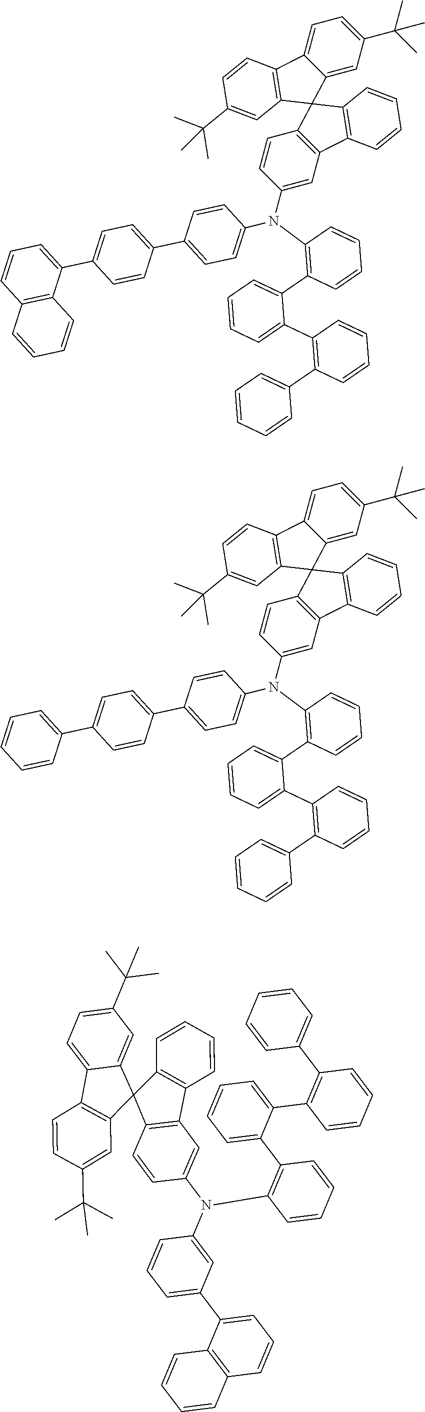 [SMILES: CC(C)(C)c1ccc2c(c1)C1(c3ccccc3-c3cc(N(c4ccc(-c5ccc(-c6cccc7ccccc67)cc5)cc4)c4ccccc4-c4ccccc4-c4ccccc4-c4ccccc4)ccc31)c1cc(C(C)(C)C)ccc1-2 ; CC(C)(C)c1ccc2c(c1)C1(c3ccccc3-c3cc(N(c4ccc(-c5ccc(-c6ccccc6)cc5)cc4)c4ccccc4-c4ccccc4-c4ccccc4-c4ccccc4)ccc31)c1cc(C(C)(C)C)ccc1-2 ; CC(C)(C)c1ccc2c(c1)C1(c3ccccc3-c3cc(N(c4cccc(-c5cccc6ccccc56)c4)c4ccccc4-c4ccccc4-c4ccccc4-c4ccccc4)ccc31)c1cc(C(C)(C)C)ccc1-2]

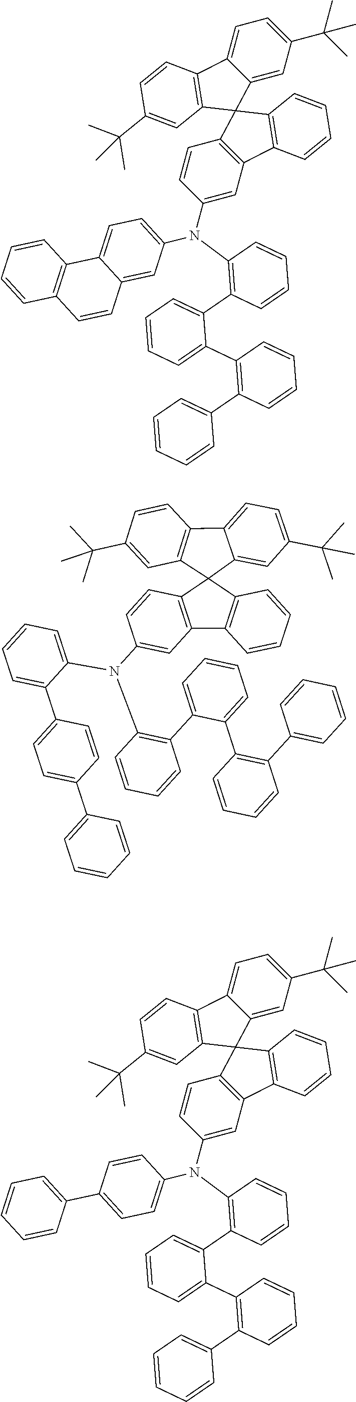

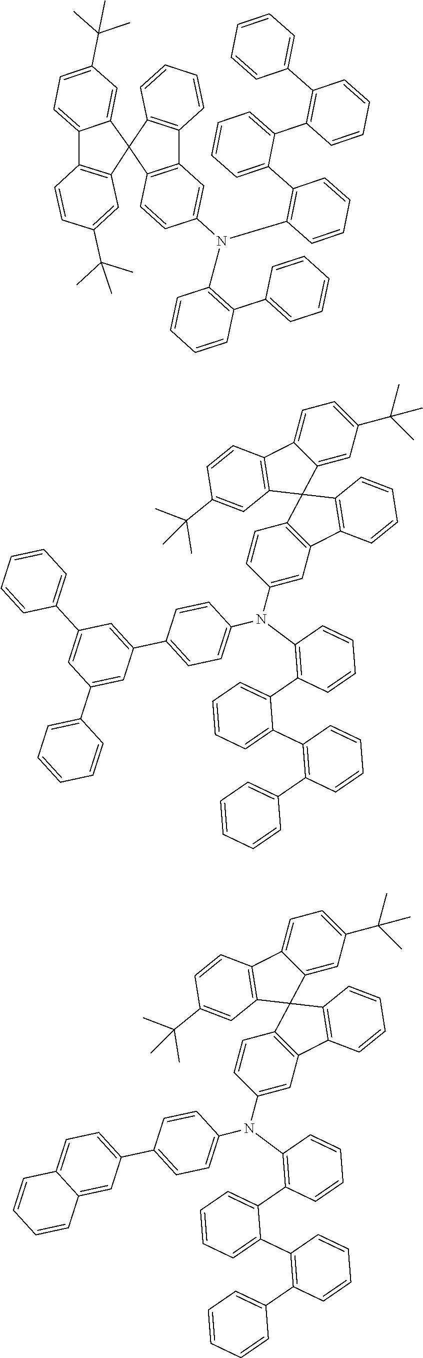 [SMILES: CC(C)(C)c1ccc2c(c1)C1(c3ccccc3-c3cc(N(c4ccccc4-c4ccccc4)c4ccccc4-c4ccccc4-c4ccccc4-c4ccccc4)ccc31)c1cc(C(C)(C)C)ccc1-2 ; CC(C)(C)c1ccc2c(c1)C1(c3ccccc3-c3cc(N(c4ccc(-c5cc(-c6ccccc6)cc(-c6ccccc6)c5)cc4)c4ccccc4-c4ccccc4-c4ccccc4-c4ccccc4)ccc31)c1cc(C(C)(C)C)ccc1-2 ; CC(C)(C)c1ccc2c(c1)C1(c3ccccc3-c3cc(N(c4ccc(-c5ccc6ccccc6c5)cc4)c4ccccc4-c4ccccc4-c4ccccc4-c4ccccc4)ccc31)c1cc(C(C)(C)C)ccc1-2]

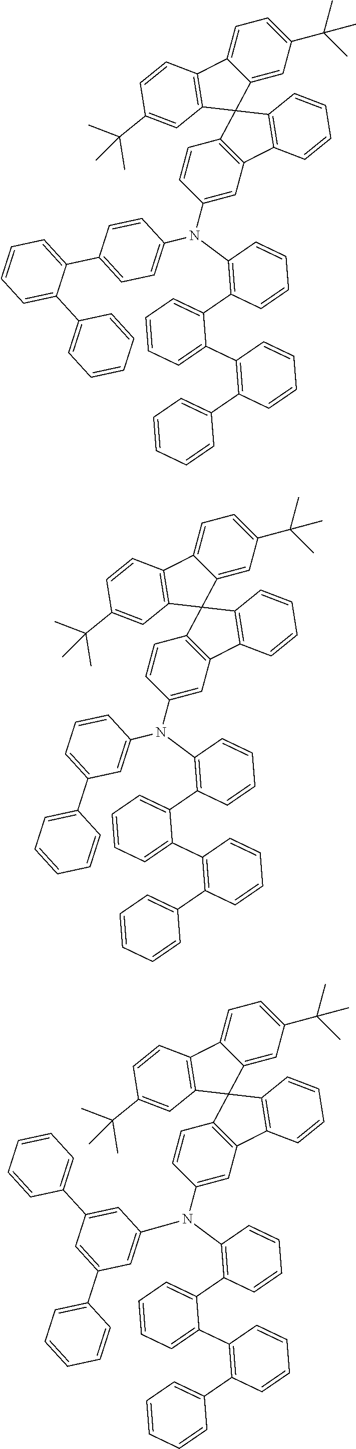 [SMILES: CC(C)(C)c1ccc2c(c1)C1(c3ccccc3-c3cc(N(c4ccc(-c5ccccc5-c5ccccc5)cc4)c4ccccc4-c4ccccc4-c4ccccc4-c4ccccc4)ccc31)c1cc(C(C)(C)C)ccc1-2 ; CC(C)(C)c1ccc2c(c1)C1(c3ccccc3-c3cc(N(c4cccc(-c5ccccc5)c4)c4ccccc4-c4ccccc4-c4ccccc4-c4ccccc4)ccc31)c1cc(C(C)(C)C)ccc1-2 ; CC(C)(C)c1ccc2c(c1)C1(c3ccccc3-c3cc(N(c4cc(-c5ccccc5)cc(-c5ccccc5)c4)c4ccccc4-c4ccccc4-c4ccccc4-c4ccccc4)ccc31)c1cc(C(C)(C)C)ccc1-2]

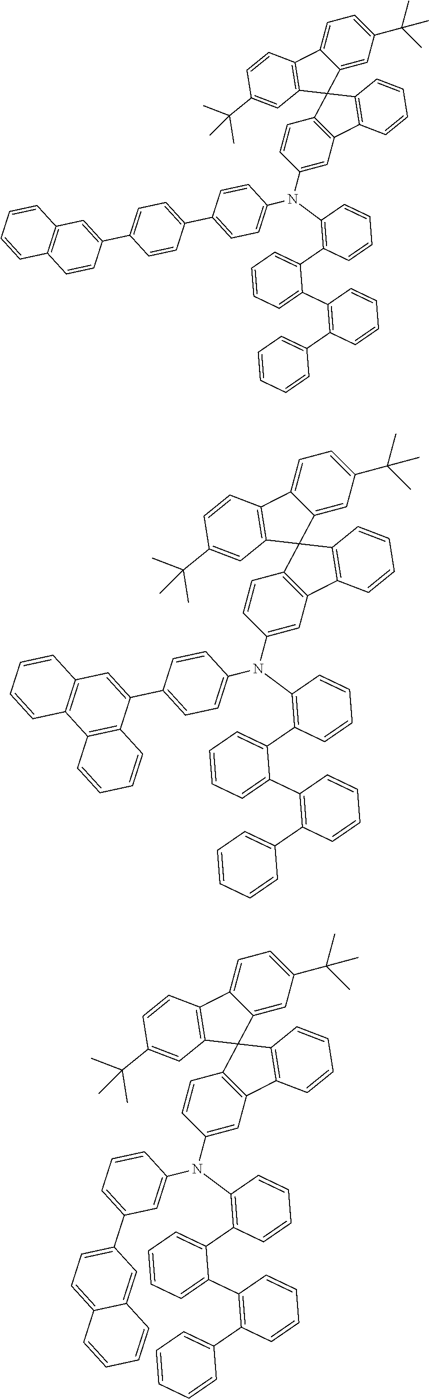 [SMILES: CC(C)(C)c1ccc2c(c1)C1(c3ccccc3-c3cc(N(c4ccc(-c5ccc(-c6ccc7ccccc7c6)cc5)cc4)c4ccccc4-c4ccccc4-c4ccccc4-c4ccccc4)ccc31)c1cc(C(C)(C)C)ccc1-2 ; CC(C)(C)c1ccc2c(c1)C1(c3ccccc3-c3cc(N(c4ccc(-c5cc6ccccc6c6ccccc56)cc4)c4ccccc4-c4ccccc4-c4ccccc4-c4ccccc4)ccc31)c1cc(C(C)(C)C)ccc1-2 ; CC(C)(C)c1ccc2c(c1)C1(c3ccccc3-c3cc(N(c4cccc(-c5ccc6ccccc6c5)c4)c4ccccc4-c4ccccc4-c4ccccc4-c4ccccc4)ccc31)c1cc(C(C)(C)C)ccc1-2]

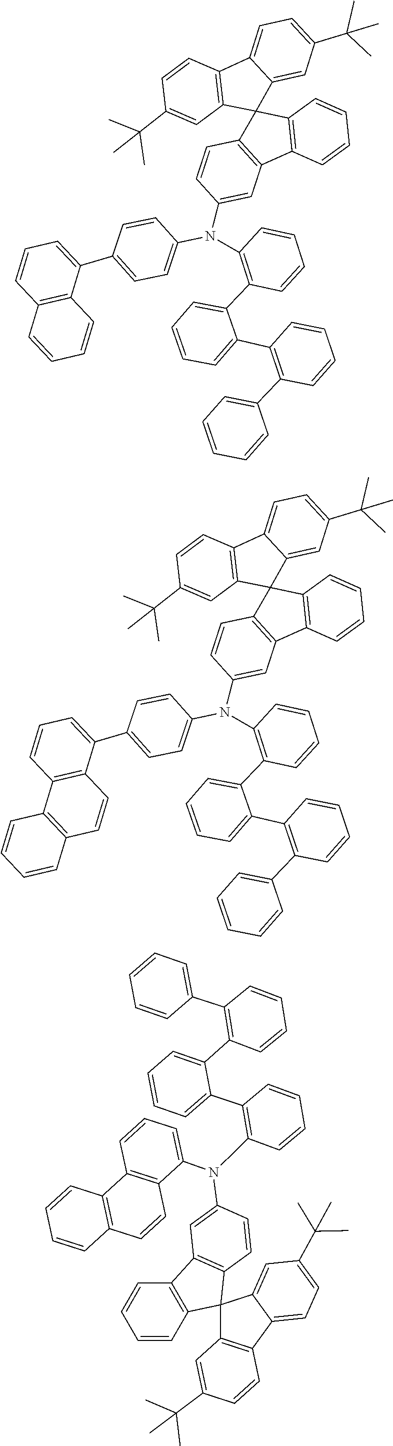 [SMILES: CC(C)(C)c1ccc2c(c1)C1(c3ccccc3-c3cc(N(c4ccc(-c5cccc6ccccc56)cc4)c4ccccc4-c4ccccc4-c4ccccc4-c4ccccc4)ccc31)c1cc(C(C)(C)C)ccc1-2 ; CC(C)(C)c1ccc2c(c1)C1(c3ccccc3-c3cc(N(c4ccc(-c5cccc6c5ccc5ccccc56)cc4)c4ccccc4-c4ccccc4-c4ccccc4-c4ccccc4)ccc31)c1cc(C(C)(C)C)ccc1-2 ; CC(C)(C)c1ccc2c(c1)C1(c3ccccc3-c3cc(N(c4ccccc4-c4ccccc4-c4ccccc4-c4ccccc4)c4cccc5c4ccc4ccccc45)ccc31)c1cc(C(C)(C)C)ccc1-2]

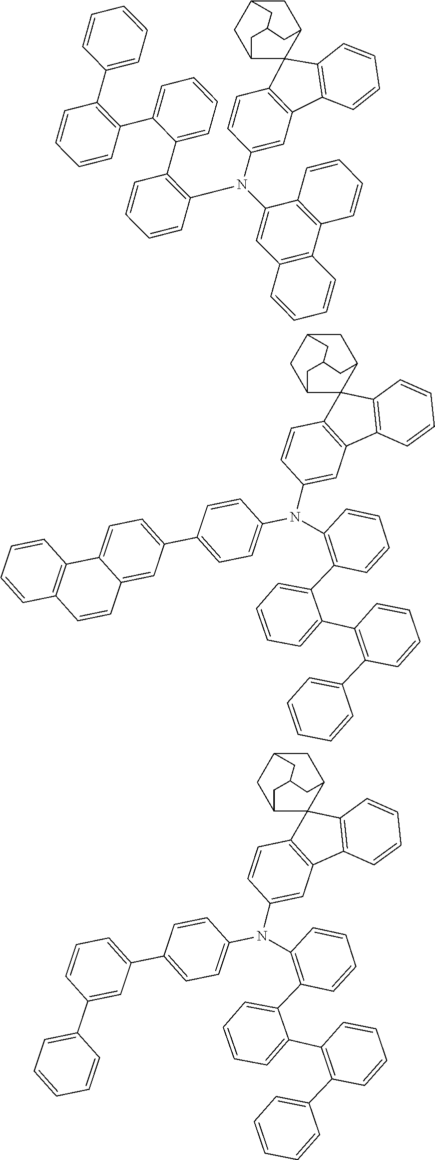

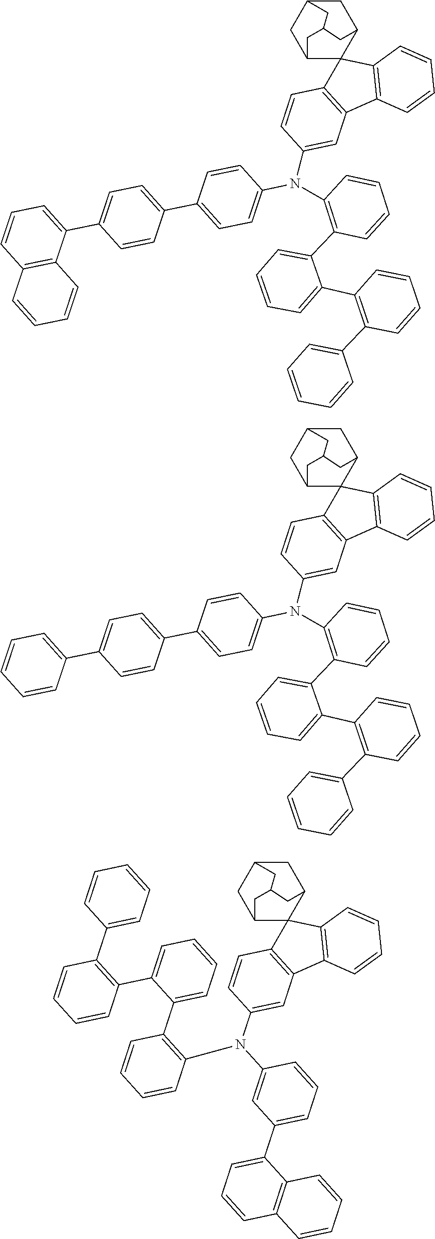

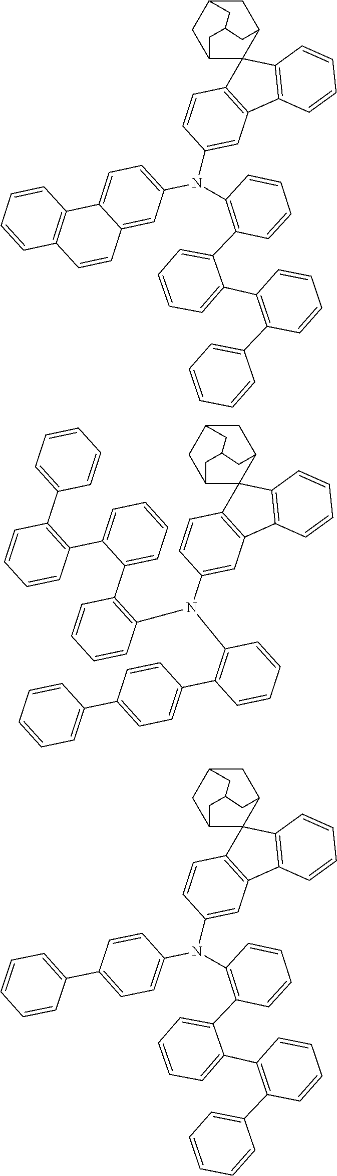

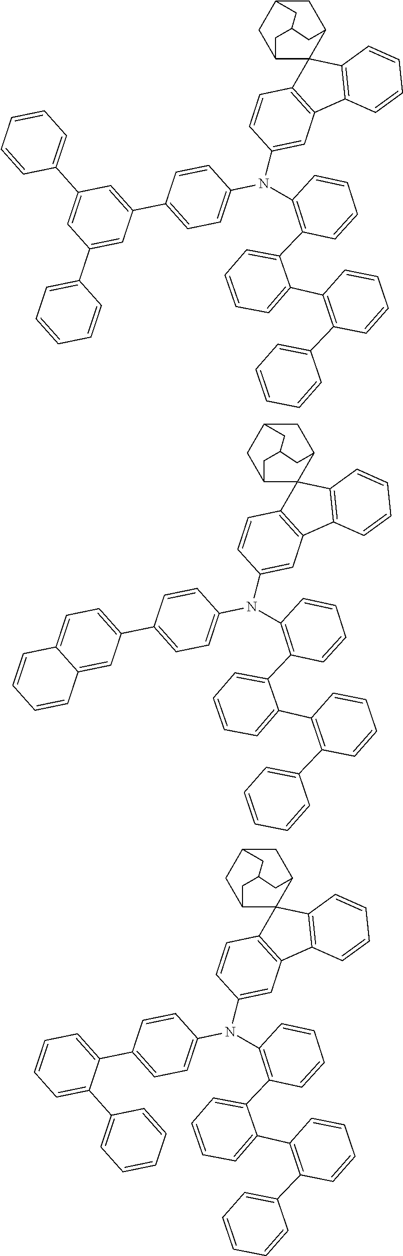

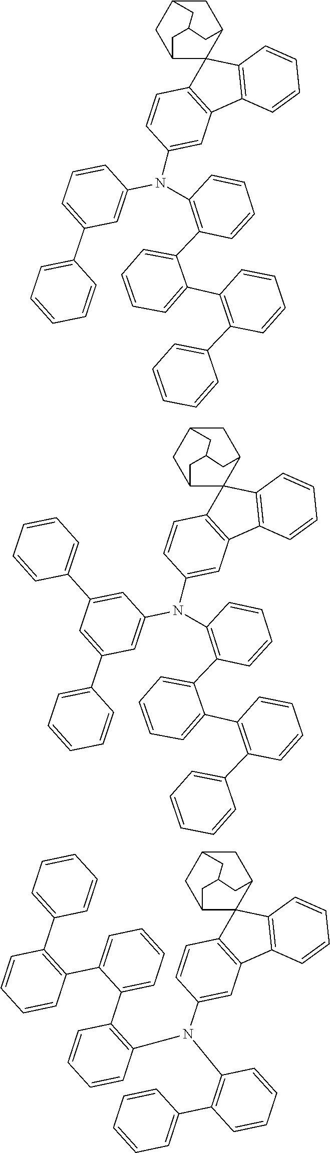

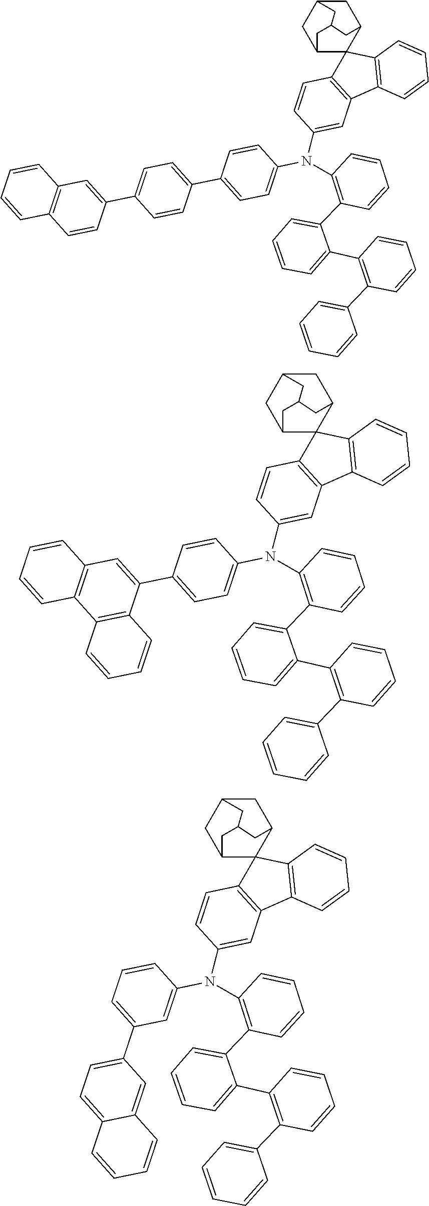

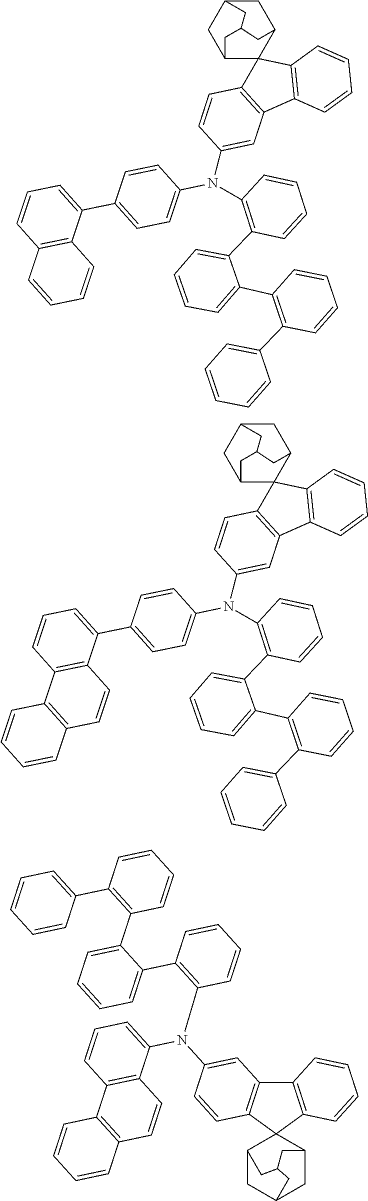

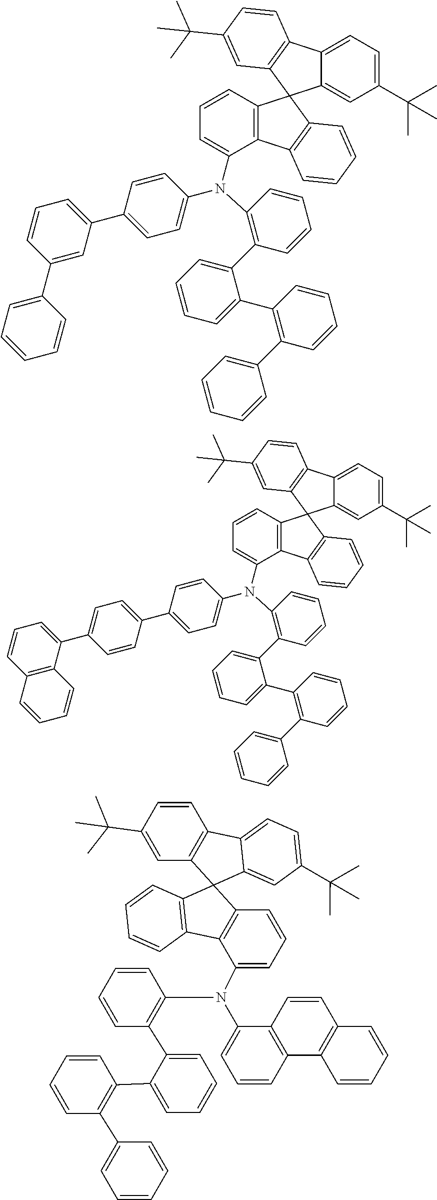

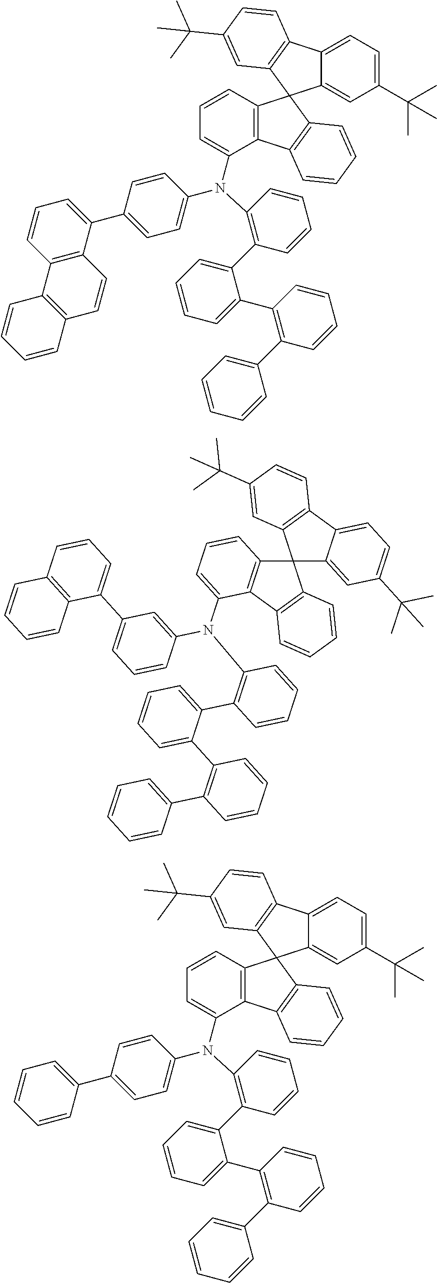

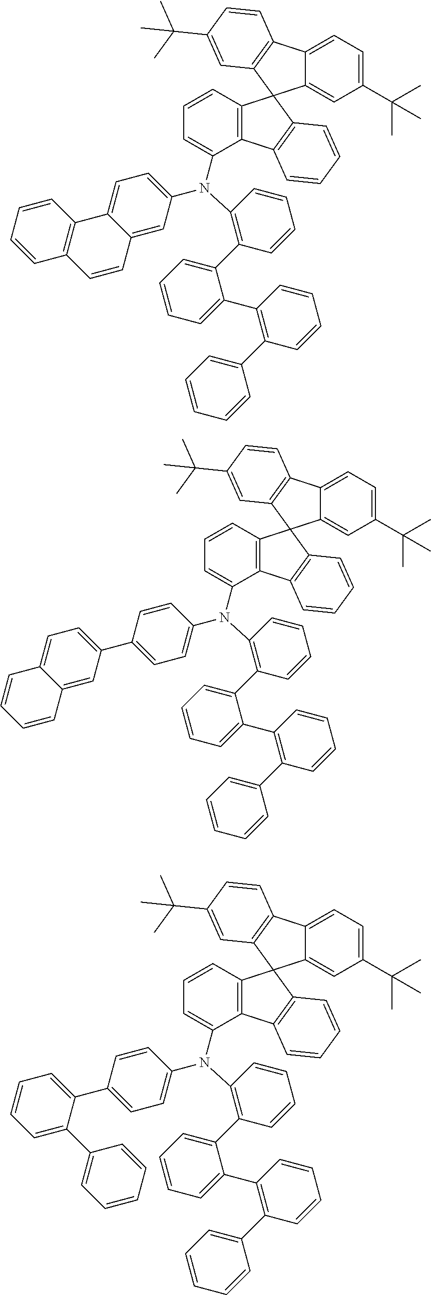

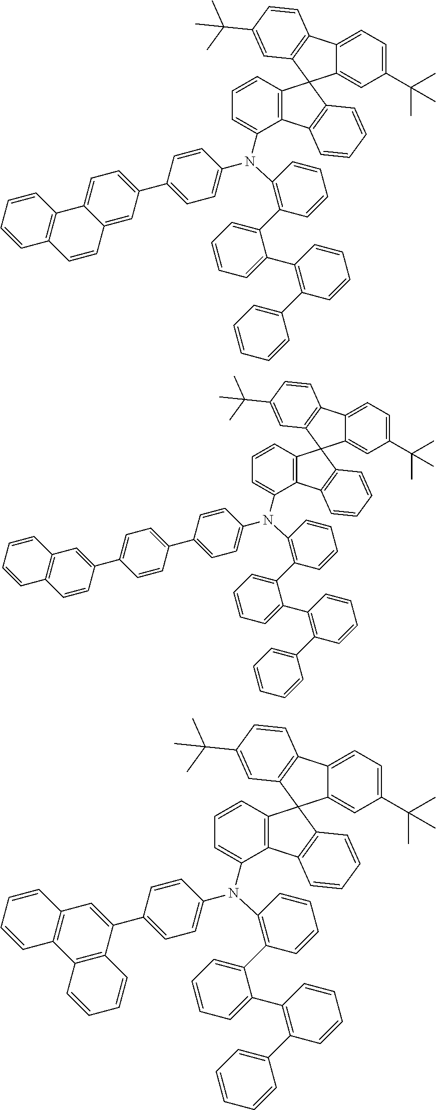

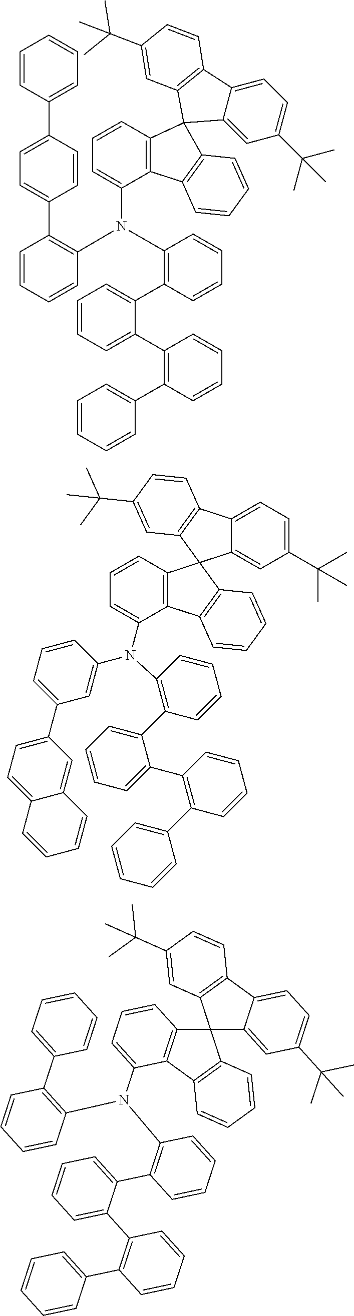

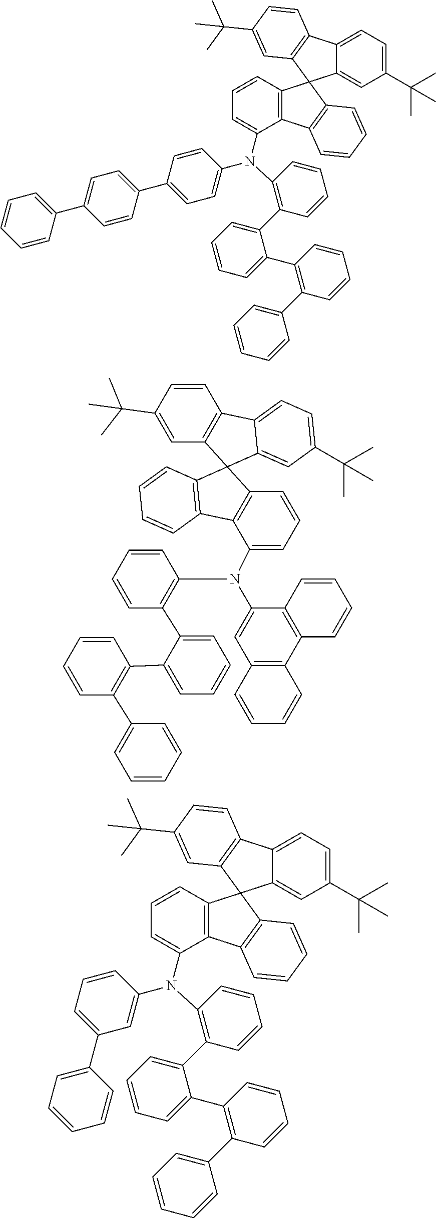

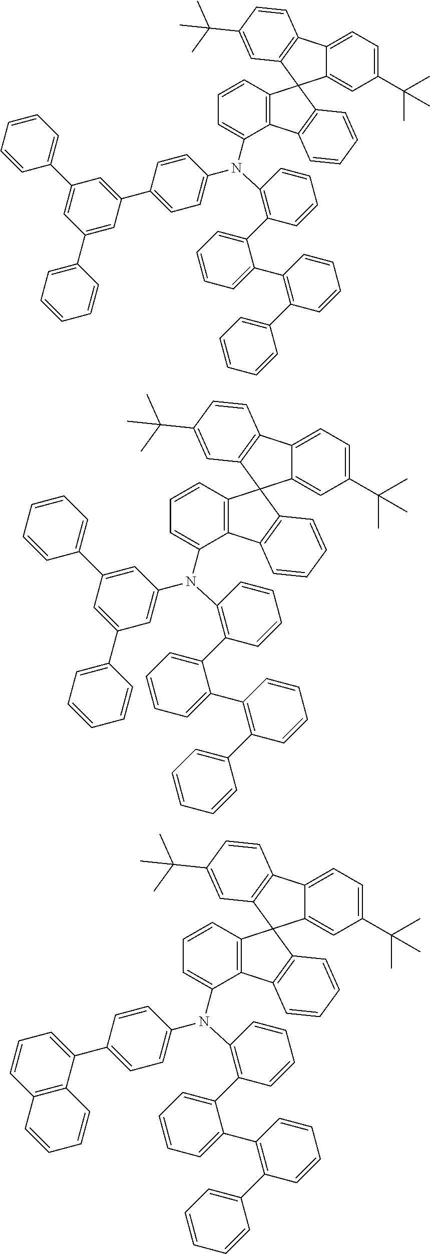

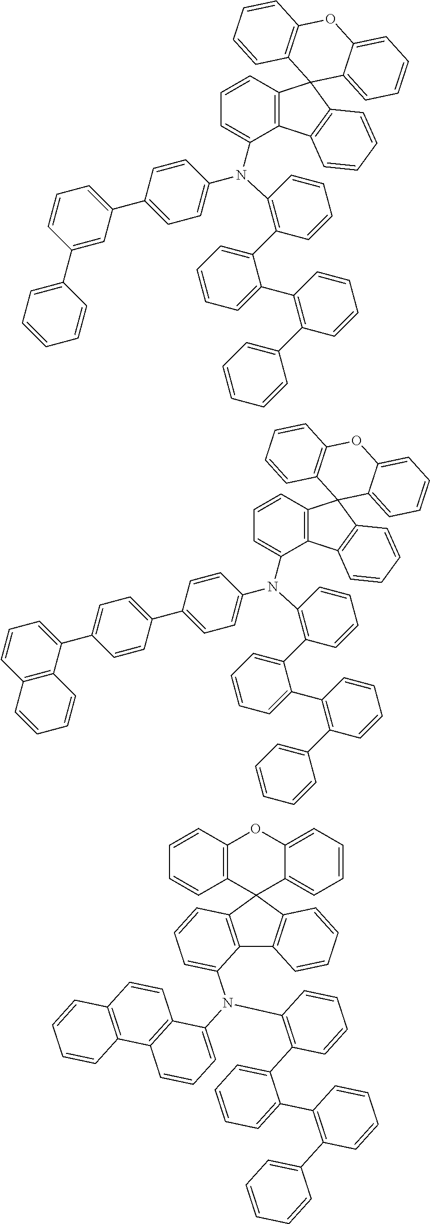

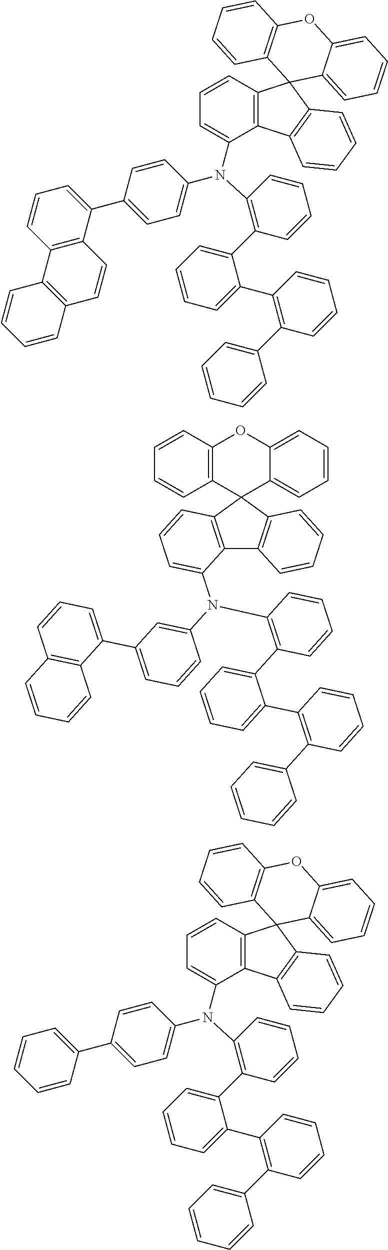

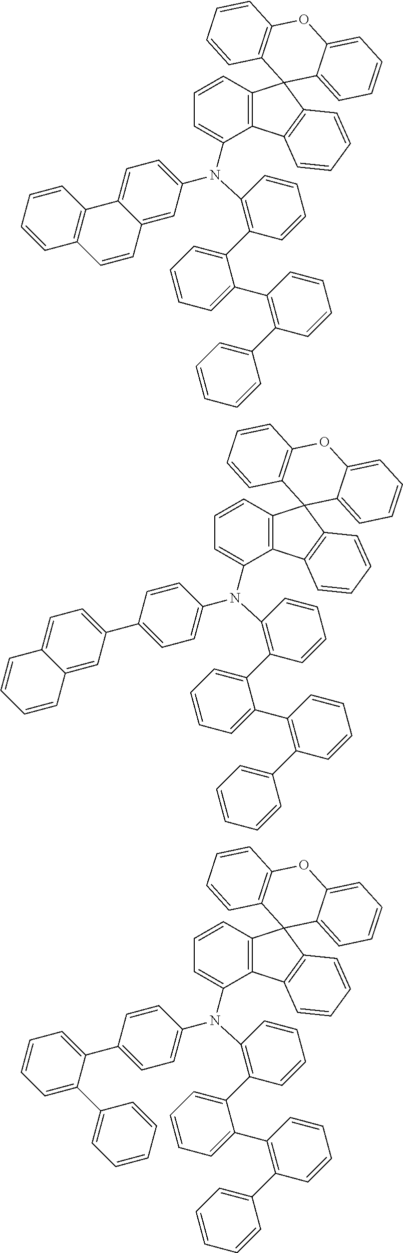

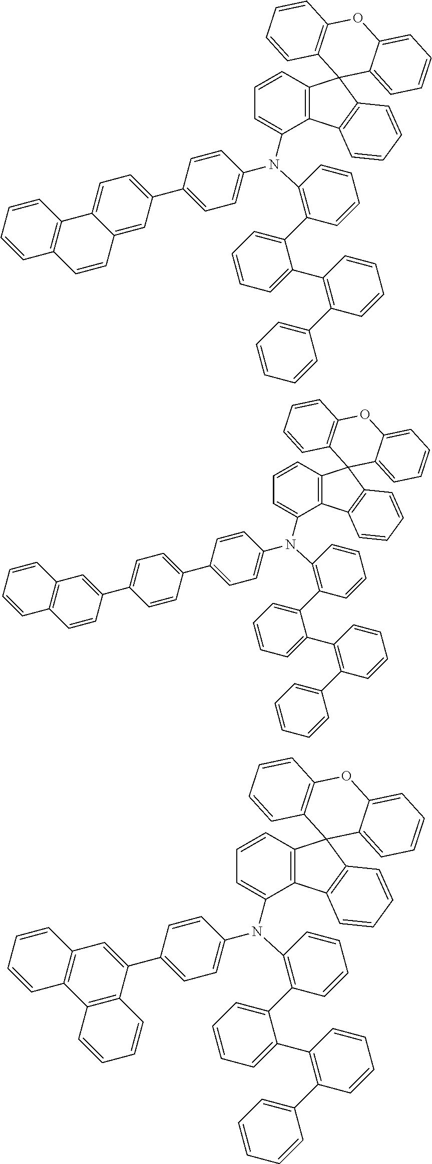

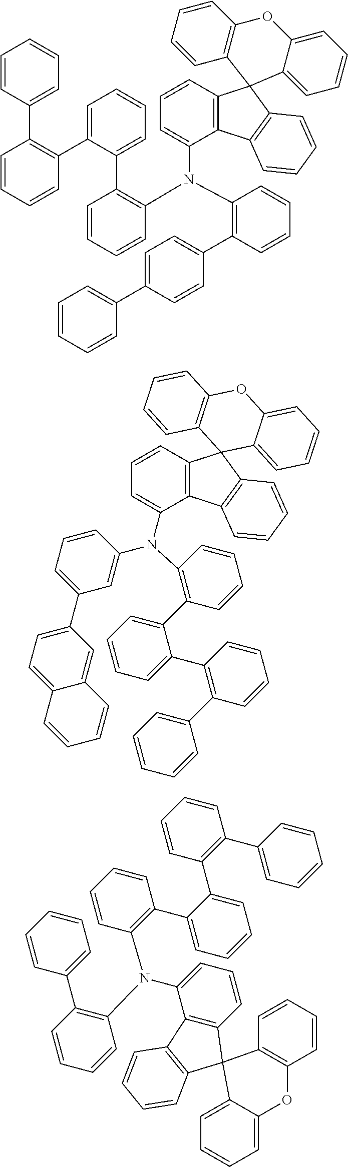

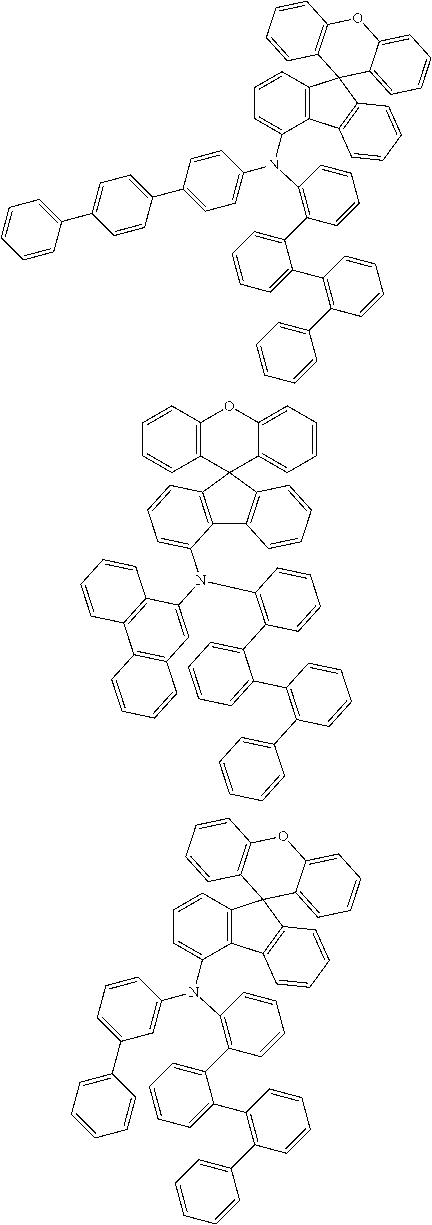

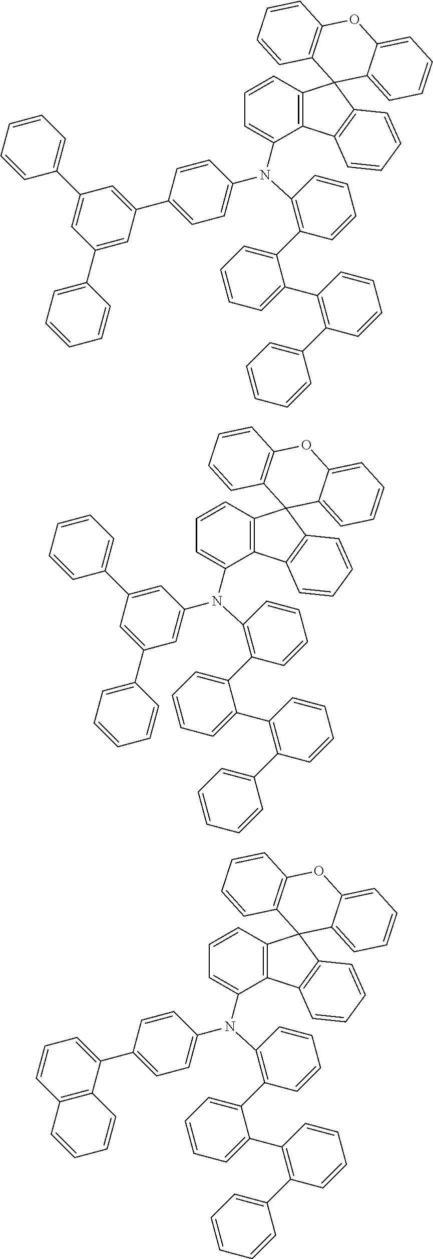

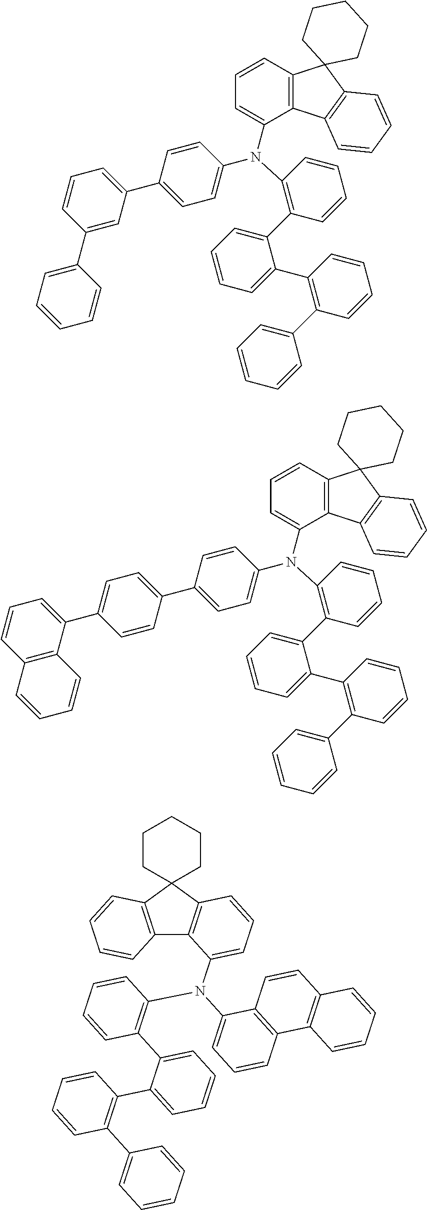 [SMILES: c1ccc(-c2cccc(-c3ccc(N(c4ccccc4-c4ccccc4-c4ccccc4-c4ccccc4)c4cccc5c4-c4ccccc4C54CCCCC4)cc3)c2)cc1 ; c1ccc(-c2ccccc2-c2ccccc2-c2ccccc2N(c2ccc(-c3ccc(-c4cccc5ccccc45)cc3)cc2)c2cccc3c2-c2ccccc2C32CCCCC2)cc1 ; c1ccc(-c2ccccc2-c2ccccc2-c2ccccc2N(c2cccc3c2-c2ccccc2C32CCCCC2)c2cccc3c2ccc2ccccc23)cc1]

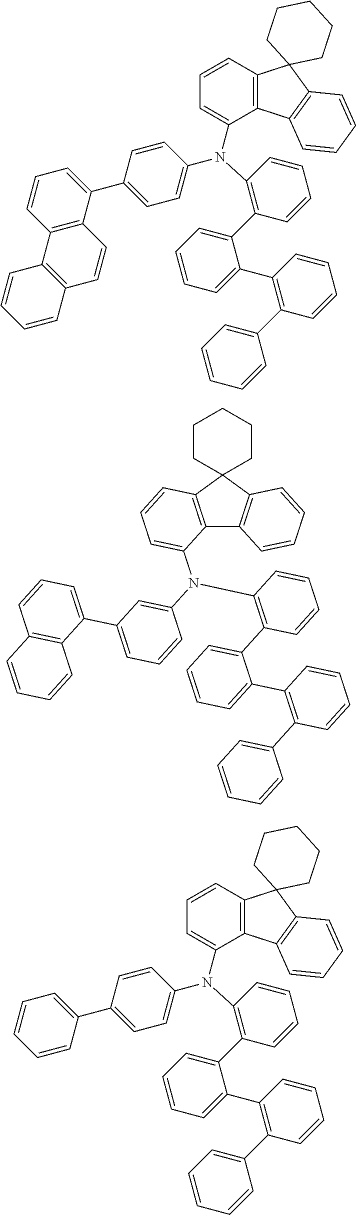

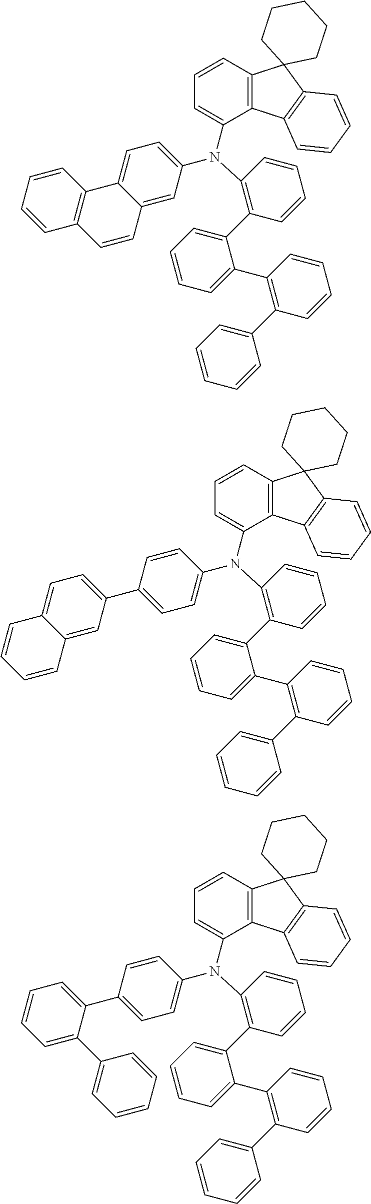

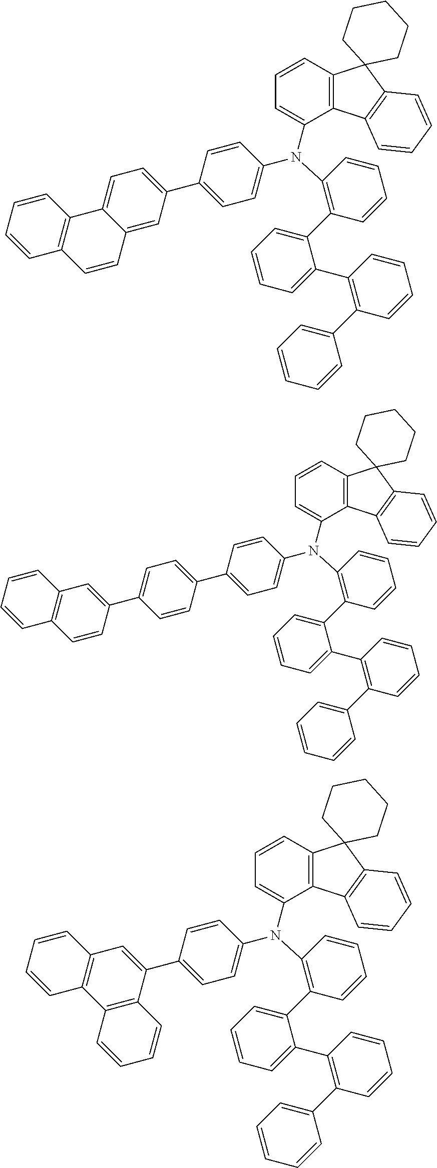

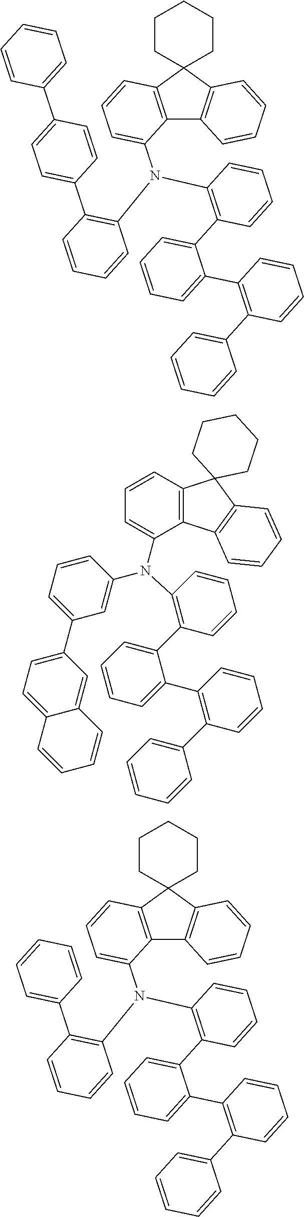

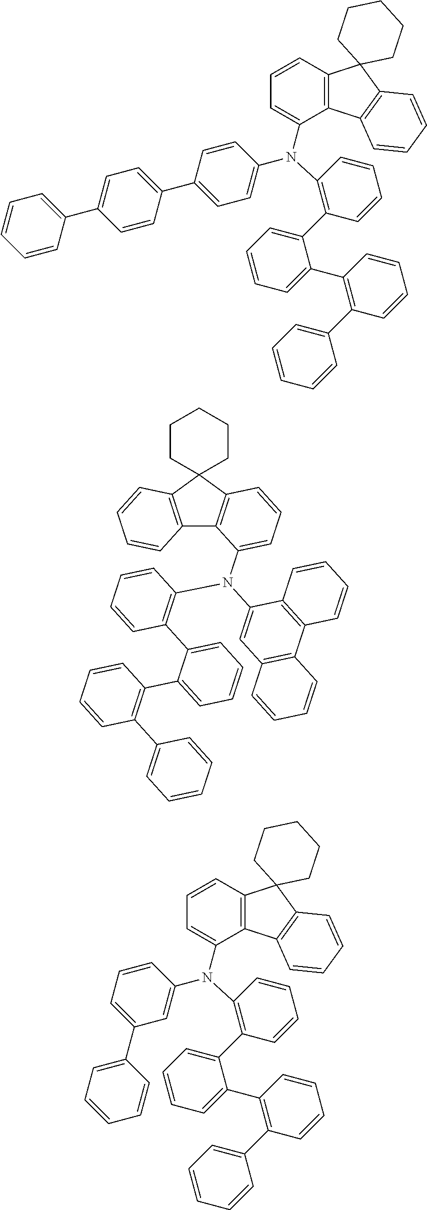 [SMILES: c1ccc(-c2ccc(-c3ccc(N(c4ccccc4-c4ccccc4-c4ccccc4-c4ccccc4)c4cccc5c4-c4ccccc4C54CCCCC4)cc3)cc2)cc1 ; c1ccc(-c2ccccc2-c2ccccc2-c2ccccc2N(c2cccc3c2-c2ccccc2C32CCCCC2)c2cc3ccccc3c3ccccc23)cc1 ; c1ccc(-c2cccc(N(c3ccccc3-c3ccccc3-c3ccccc3-c3ccccc3)c3cccc4c3-c3ccccc3C43CCCCC3)c2)cc1]

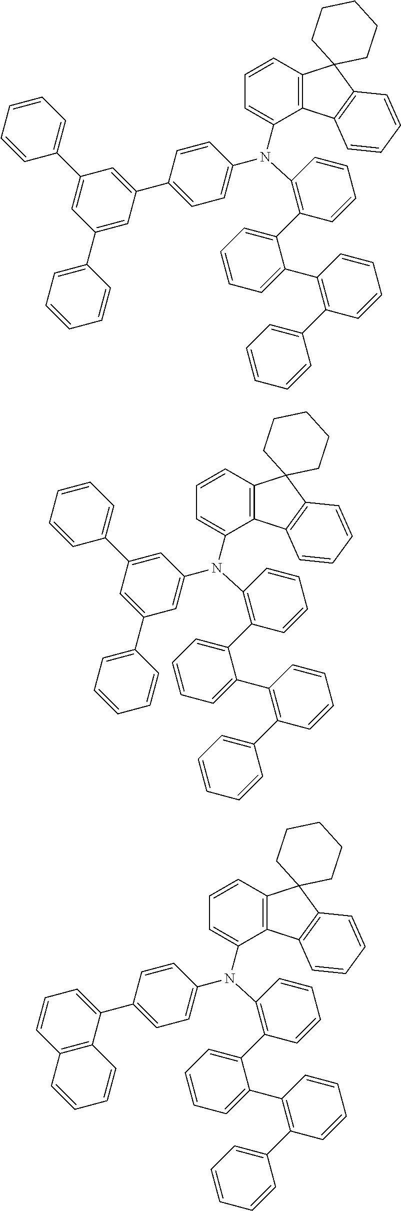

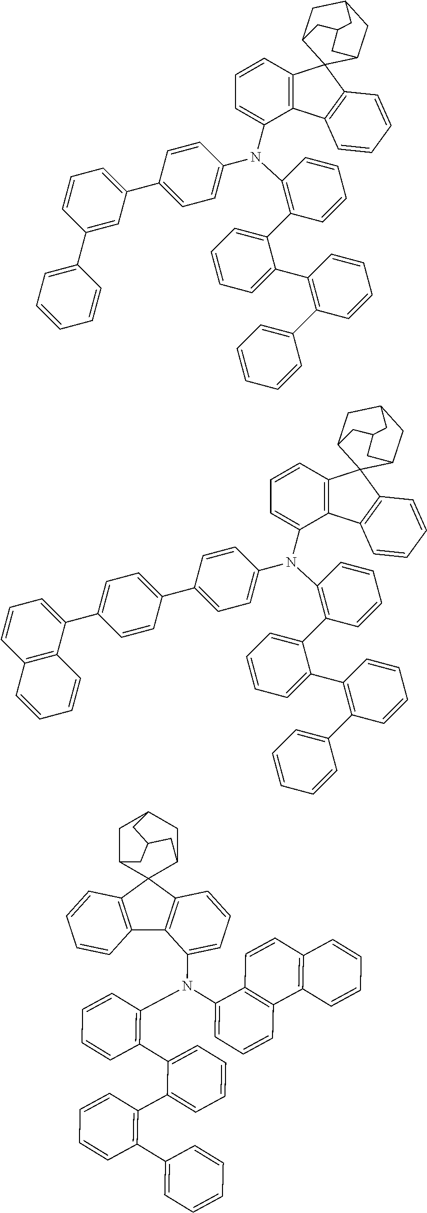 [SMILES: c1ccc(-c2cccc(-c3ccc(N(c4ccccc4-c4ccccc4-c4ccccc4-c4ccccc4)c4cccc5c4-c4ccccc4C54C5CC6CC(C5)CC4C6)cc3)c2)cc1 ; c1ccc(-c2ccccc2-c2ccccc2-c2ccccc2N(c2ccc(-c3ccc(-c4cccc5ccccc45)cc3)cc2)c2cccc3c2-c2ccccc2C32C3CC4CC(C3)CC2C4)cc1 ; c1ccc(-c2ccccc2-c2ccccc2-c2ccccc2N(c2cccc3c2-c2ccccc2C32C3CC4CC(C3)CC2C4)c2cccc3c2ccc2ccccc23)cc1]

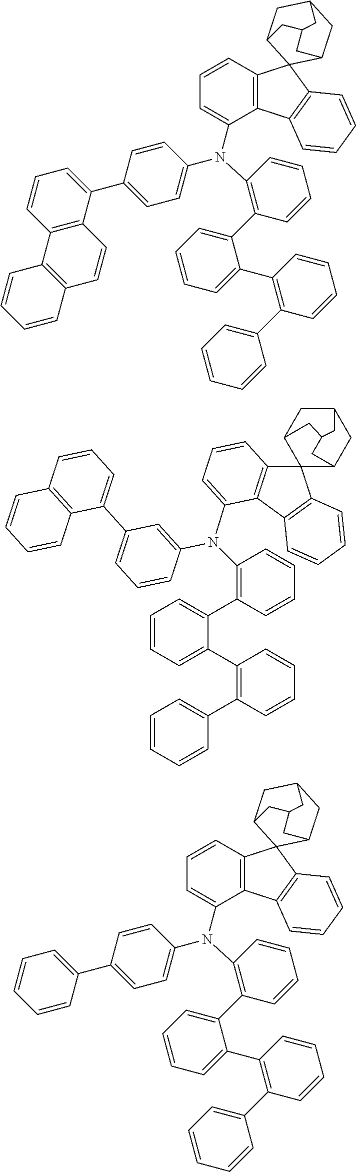 [SMILES: c1ccc(-c2ccccc2-c2ccccc2-c2ccccc2N(c2ccc(-c3cccc4c3ccc3ccccc34)cc2)c2cccc3c2-c2ccccc2C32C3CC4CC(C3)CC2C4)cc1 ; c1ccc(-c2ccccc2-c2ccccc2-c2ccccc2N(c2cccc(-c3cccc4ccccc34)c2)c2cccc3c2-c2ccccc2C32C3CC4CC(C3)CC2C4)cc1 ; c1ccc(-c2ccc(N(c3ccccc3-c3ccccc3-c3ccccc3-c3ccccc3)c3cccc4c3-c3ccccc3C43C4CC5CC(C4)CC3C5)cc2)cc1]

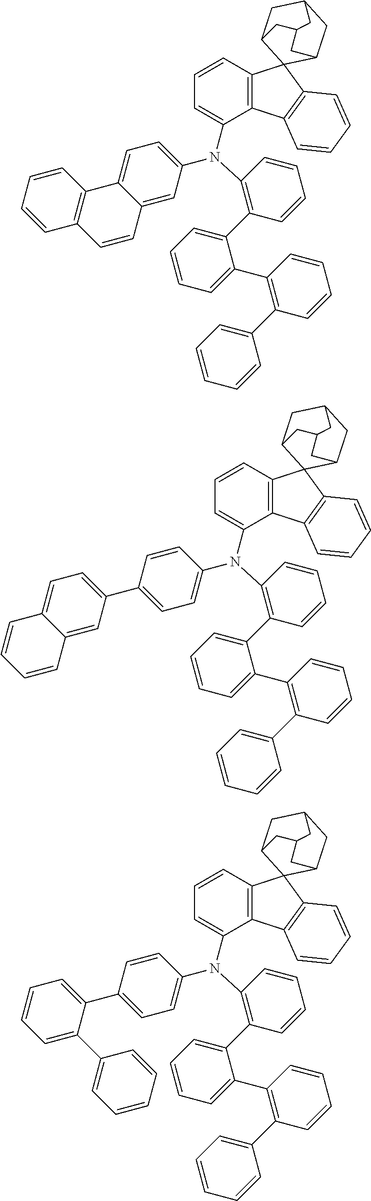

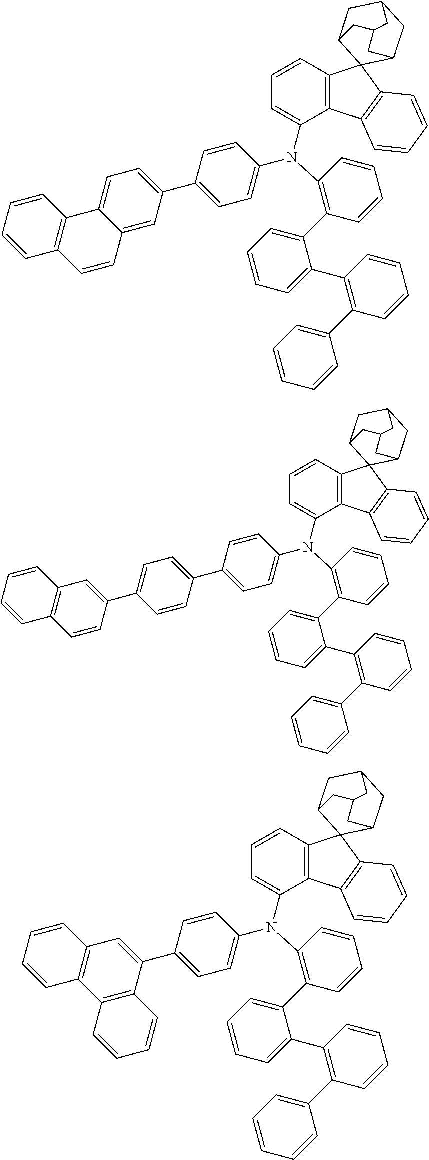

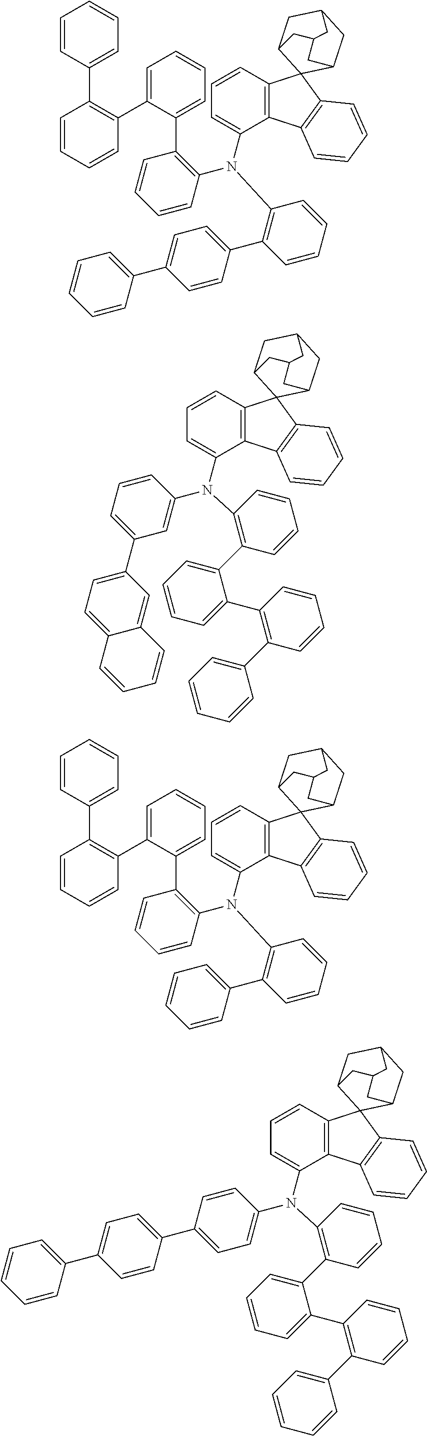 [SMILES: c1ccc(-c2ccc(-c3ccccc3N(c3ccccc3-c3ccccc3-c3ccccc3-c3ccccc3)c3cccc4c3-c3ccccc3C43C4CC5CC(C4)CC3C5)cc2)cc1 ; c1ccc(-c2ccccc2-c2ccccc2-c2ccccc2N(c2cccc(-c3ccc4ccccc4c3)c2)c2cccc3c2-c2ccccc2C32C3CC4CC(C3)CC2C4)cc1 ; c1ccc(-c2ccccc2-c2ccccc2-c2ccccc2N(c2ccccc2-c2ccccc2)c2cccc3c2-c2ccccc2C32C3CC4CC(C3)CC2C4)cc1 ; c1ccc(-c2ccc(-c3ccc(N(c4ccccc4-c4ccccc4-c4ccccc4-c4ccccc4)c4cccc5c4-c4ccccc4C54C5CC6CC(C5)CC4C6)cc3)cc2)cc1]

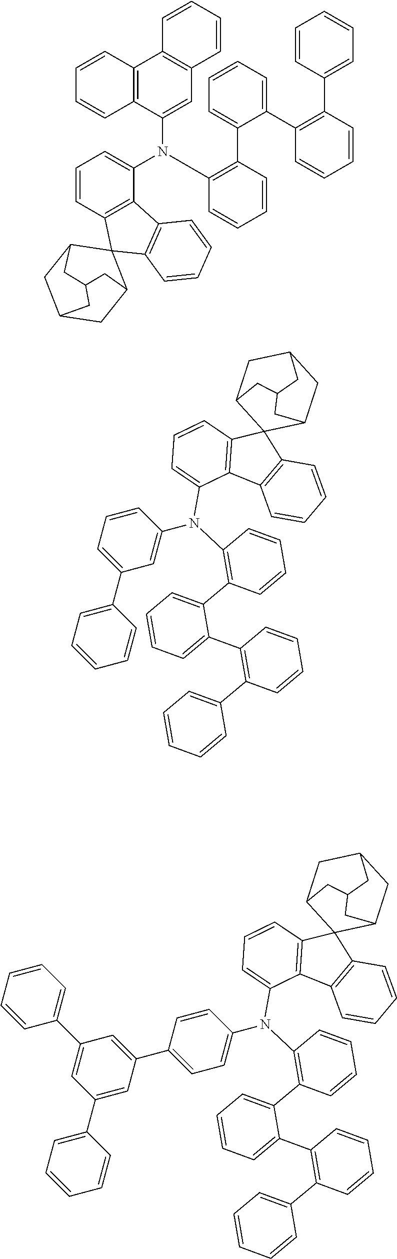 [SMILES: c1ccc(-c2ccccc2-c2ccccc2-c2ccccc2N(c2cccc3c2-c2ccccc2C32C3CC4CC(C3)CC2C4)c2cc3ccccc3c3ccccc23)cc1 ; c1ccc(-c2cccc(N(c3ccccc3-c3ccccc3-c3ccccc3-c3ccccc3)c3cccc4c3-c3ccccc3C43C4CC5CC(C4)CC3C5)c2)cc1 ; c1ccc(-c2cc(-c3ccccc3)cc(-c3ccc(N(c4ccccc4-c4ccccc4-c4ccccc4-c4ccccc4)c4cccc5c4-c4ccccc4C54C5CC6CC(C5)CC4C6)cc3)c2)cc1]

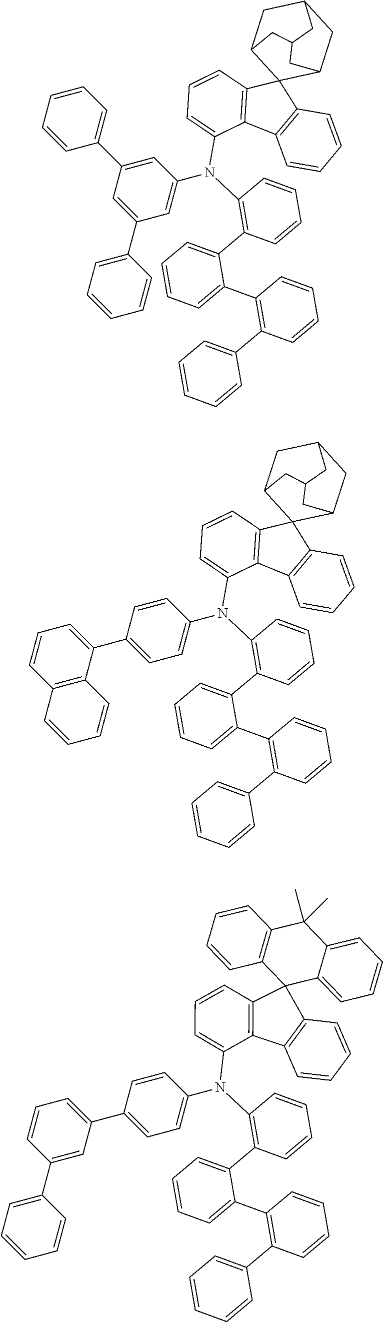 [SMILES: c1ccc(-c2cc(-c3ccccc3)cc(N(c3ccccc3-c3ccccc3-c3ccccc3-c3ccccc3)c3cccc4c3-c3ccccc3C43C4CC5CC(C4)CC3C5)c2)cc1 ; c1ccc(-c2ccccc2-c2ccccc2-c2ccccc2N(c2ccc(-c3cccc4ccccc34)cc2)c2cccc3c2-c2ccccc2C32C3CC4CC(C3)CC2C4)cc1 ; CC1(C)c2ccccc2C2(c3ccccc3-c3c(N(c4ccc(-c5cccc(-c6ccccc6)c5)cc4)c4ccccc4-c4ccccc4-c4ccccc4-c4ccccc4)cccc32)c2ccccc21]

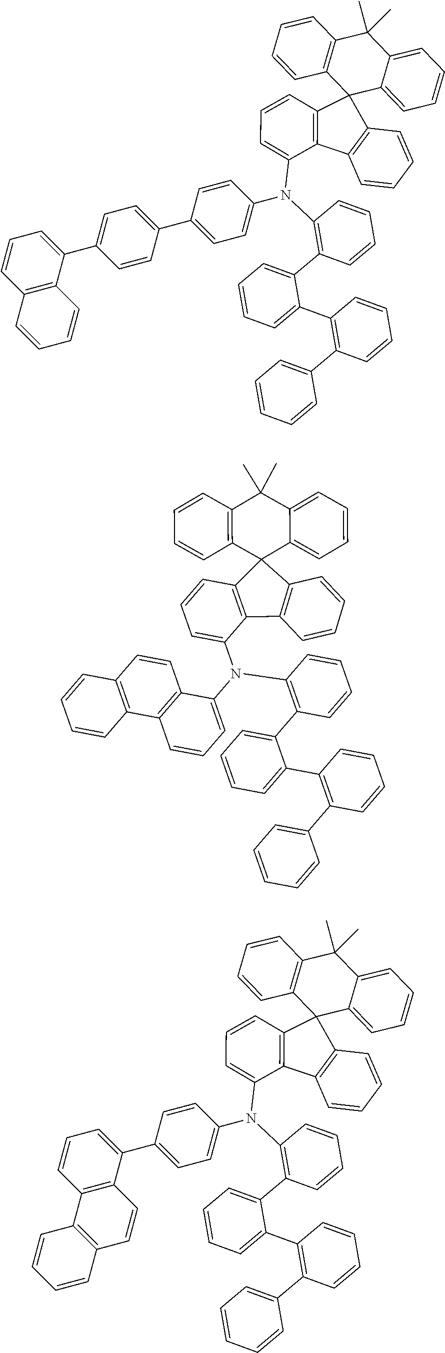

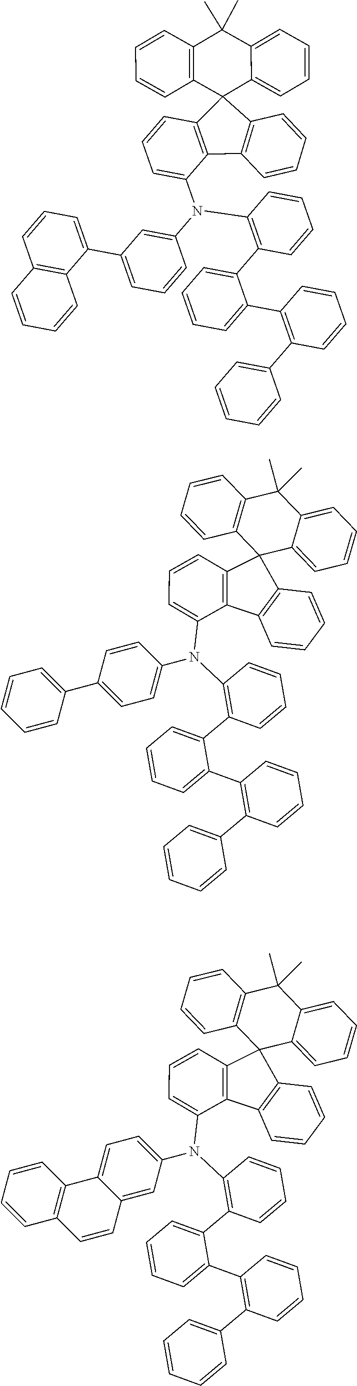

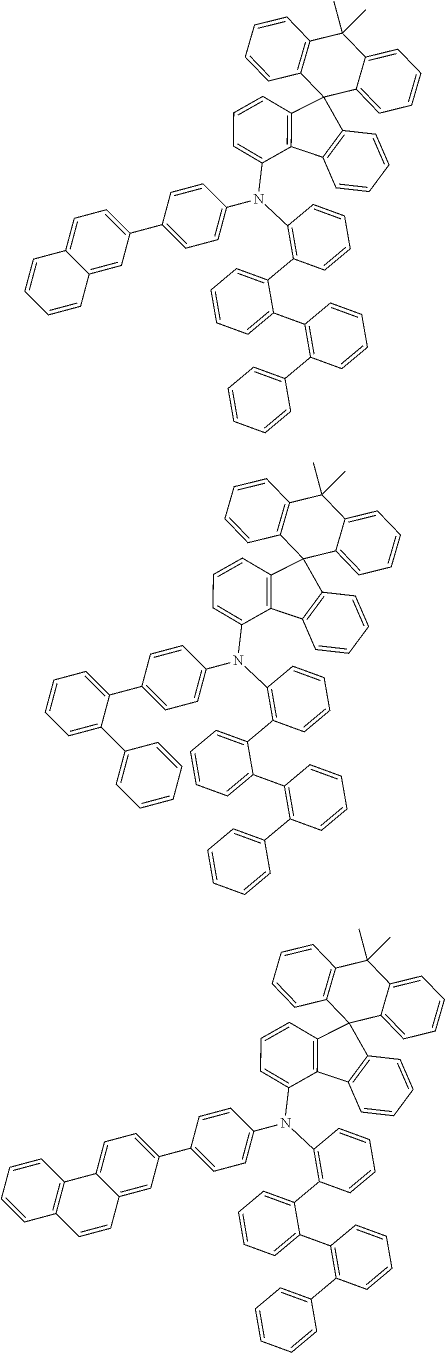

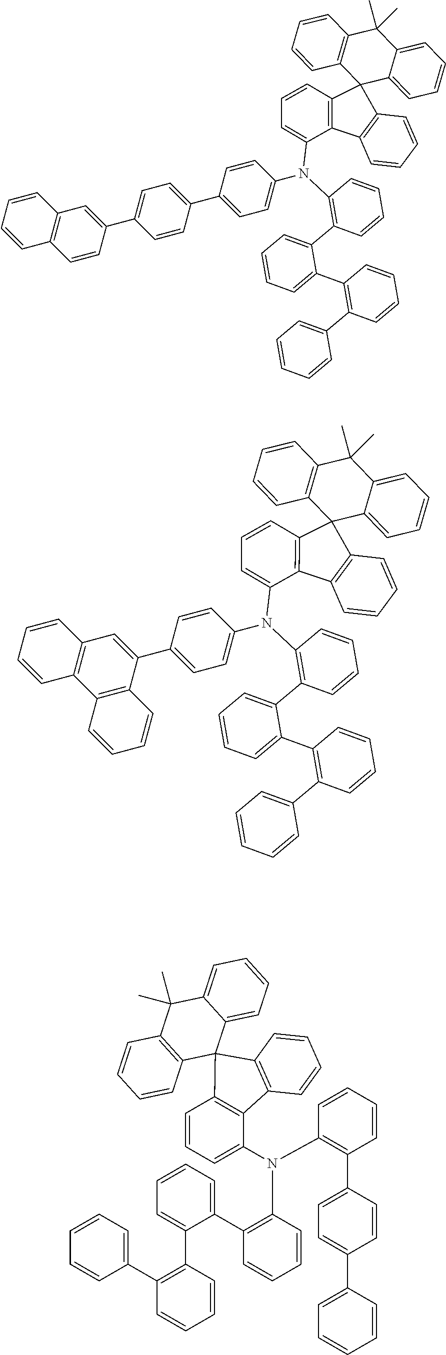 [SMILES: CC1(C)c2ccccc2C2(c3ccccc3-c3c(N(c4ccc(-c5ccc(-c6ccc7ccccc7c6)cc5)cc4)c4ccccc4-c4ccccc4-c4ccccc4-c4ccccc4)cccc32)c2ccccc21 ; CC1(C)c2ccccc2C2(c3ccccc3-c3c(N(c4ccc(-c5cc6ccccc6c6ccccc56)cc4)c4ccccc4-c4ccccc4-c4ccccc4-c4ccccc4)cccc32)c2ccccc21 ; CC1(C)c2ccccc2C2(c3ccccc3-c3c(N(c4ccccc4-c4ccc(-c5ccccc5)cc4)c4ccccc4-c4ccccc4-c4ccccc4-c4ccccc4)cccc32)c2ccccc21]

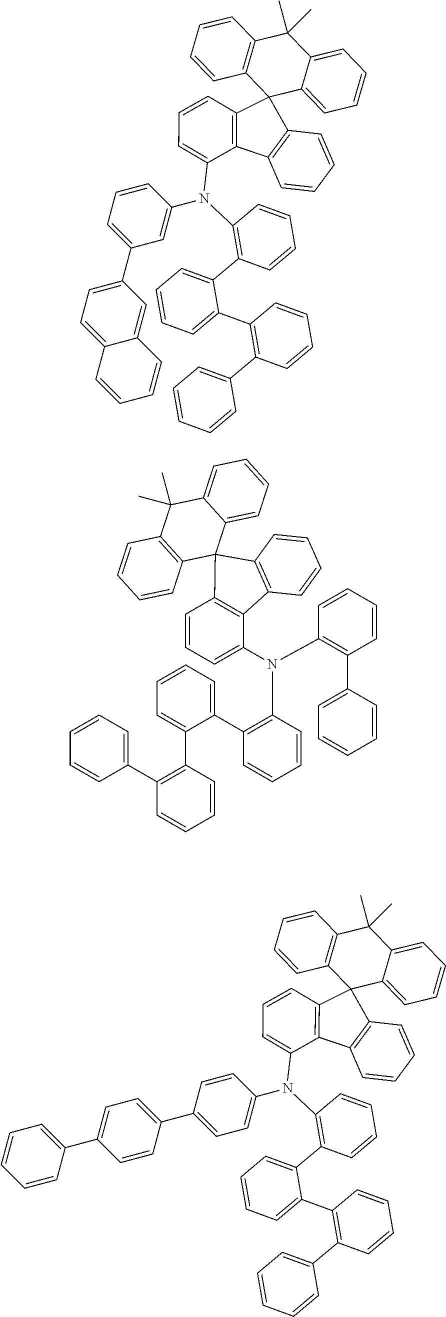

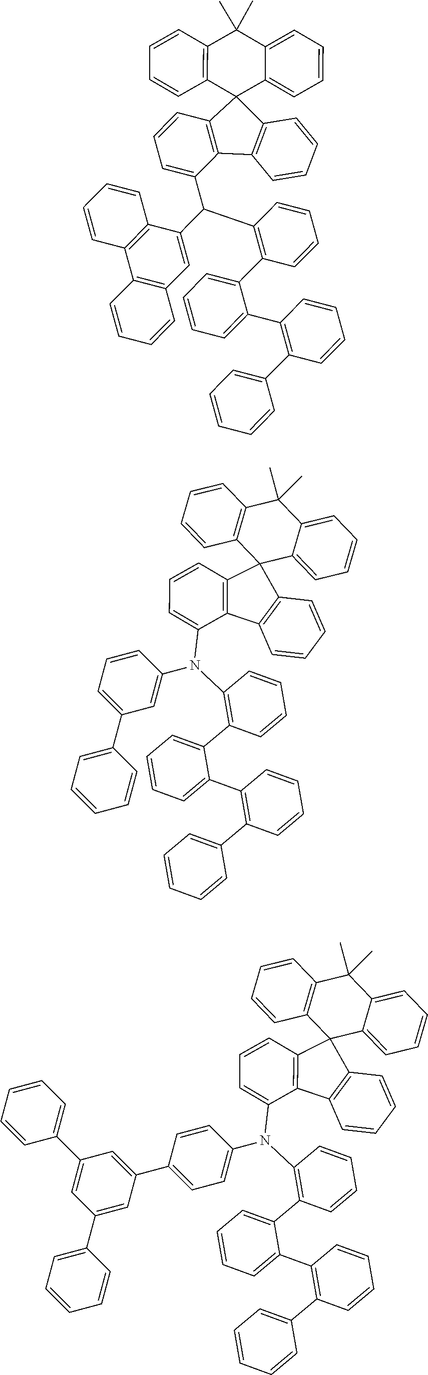

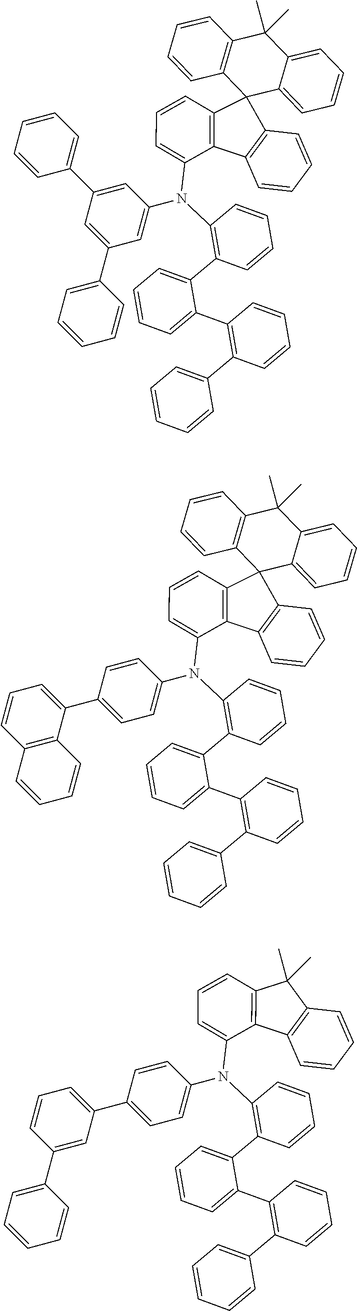 [SMILES: CC1(C)c2ccccc2C2(c3ccccc3-c3c(N(c4cc(-c5ccccc5)cc(-c5ccccc5)c4)c4ccccc4-c4ccccc4-c4ccccc4-c4ccccc4)cccc32)c2ccccc21 ; CC1(C)c2ccccc2C2(c3ccccc3-c3c(N(c4ccc(-c5cccc6ccccc56)cc4)c4ccccc4-c4ccccc4-c4ccccc4-c4ccccc4)cccc32)c2ccccc21 ; CC1(C)c2ccccc2-c2c(N(c3ccc(-c4cccc(-c5ccccc5)c4)cc3)c3ccccc3-c3ccccc3-c3ccccc3-c3ccccc3)cccc21]

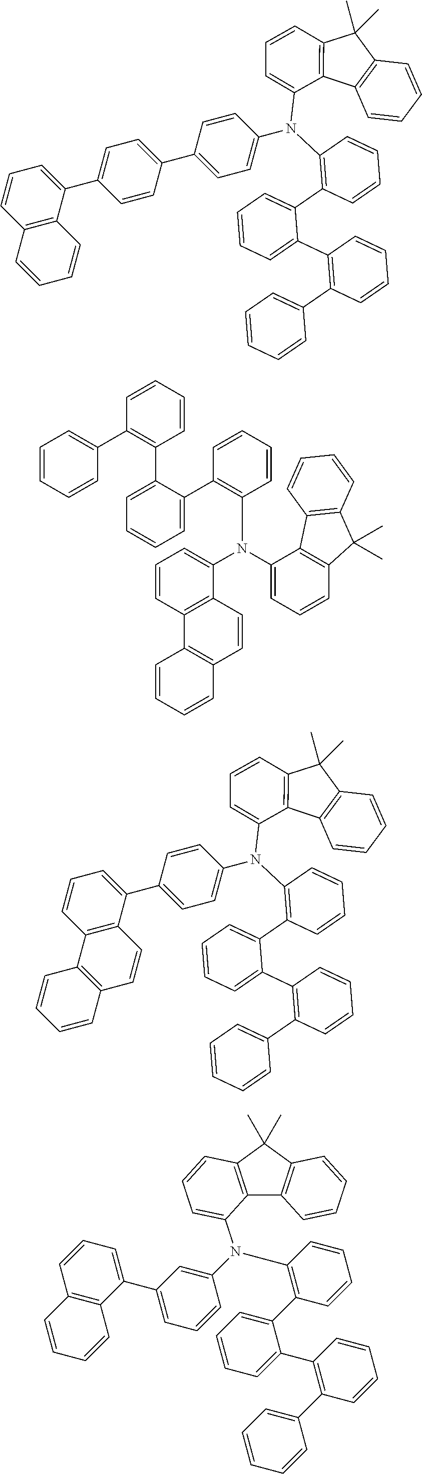

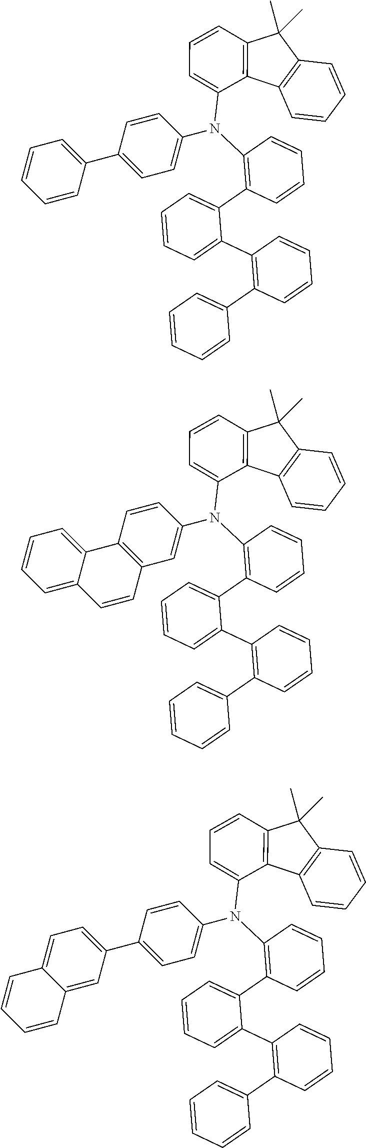 [SMILES: CC1(C)c2ccccc2-c2c(N(c3ccc(-c4ccccc4)cc3)c3ccccc3-c3ccccc3-c3ccccc3-c3ccccc3)cccc21 ; CC1(C)c2ccccc2-c2c(N(c3ccc4c(ccc5ccccc54)c3)c3ccccc3-c3ccccc3-c3ccccc3-c3ccccc3)cccc21 ; CC1(C)c2ccccc2-c2c(N(c3ccc(-c4ccc5ccccc5c4)cc3)c3ccccc3-c3ccccc3-c3ccccc3-c3ccccc3)cccc21]

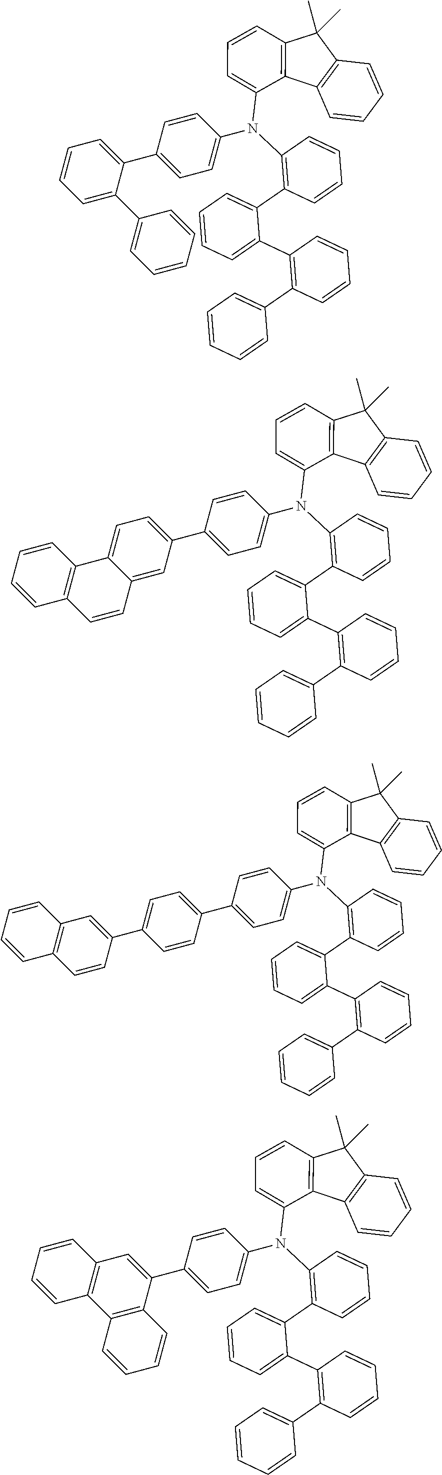

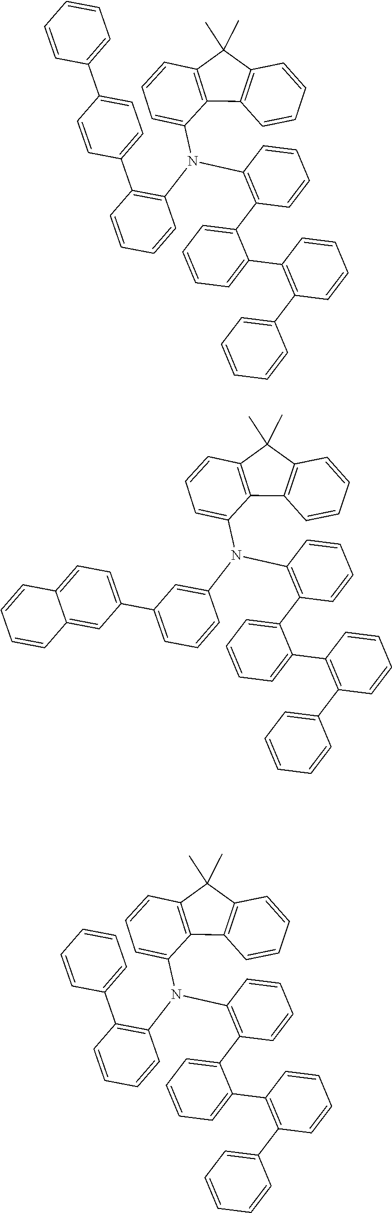

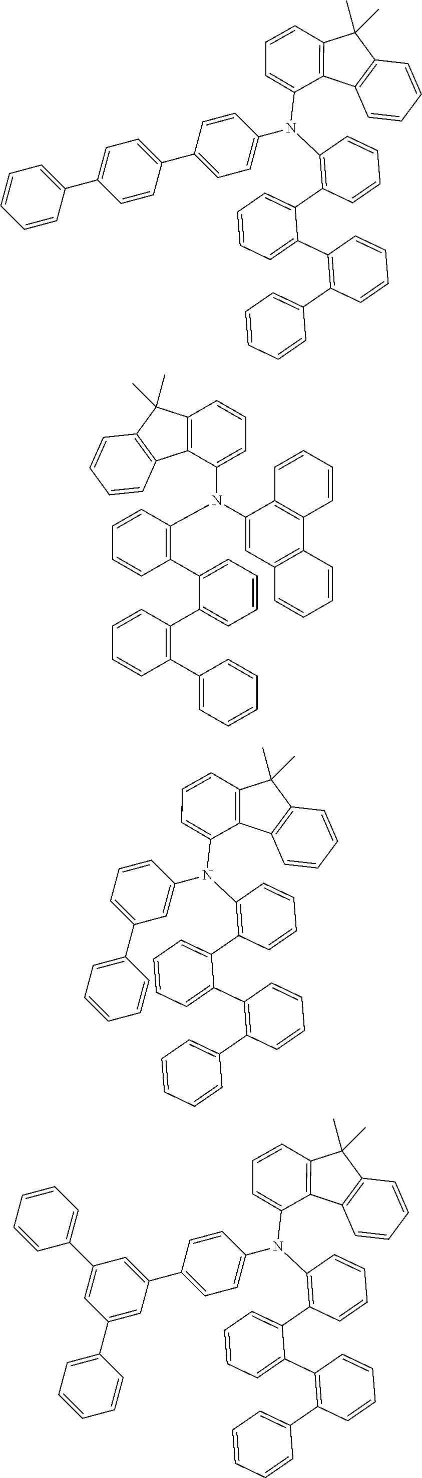

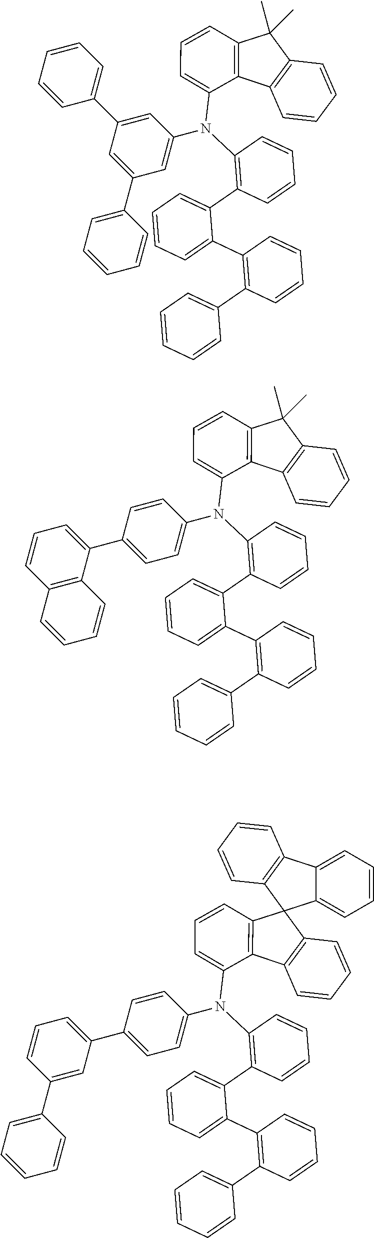 [SMILES: CC1(C)c2ccccc2-c2c(N(c3cc(-c4ccccc4)cc(-c4ccccc4)c3)c3ccccc3-c3ccccc3-c3ccccc3-c3ccccc3)cccc21 ; CC1(C)c2ccccc2-c2c(N(c3ccc(-c4cccc5ccccc45)cc3)c3ccccc3-c3ccccc3-c3ccccc3-c3ccccc3)cccc21 ; c1ccc(-c2cccc(-c3ccc(N(c4ccccc4-c4ccccc4-c4ccccc4-c4ccccc4)c4cccc5c4-c4ccccc4C54c5ccccc5-c5ccccc54)cc3)c2)cc1]

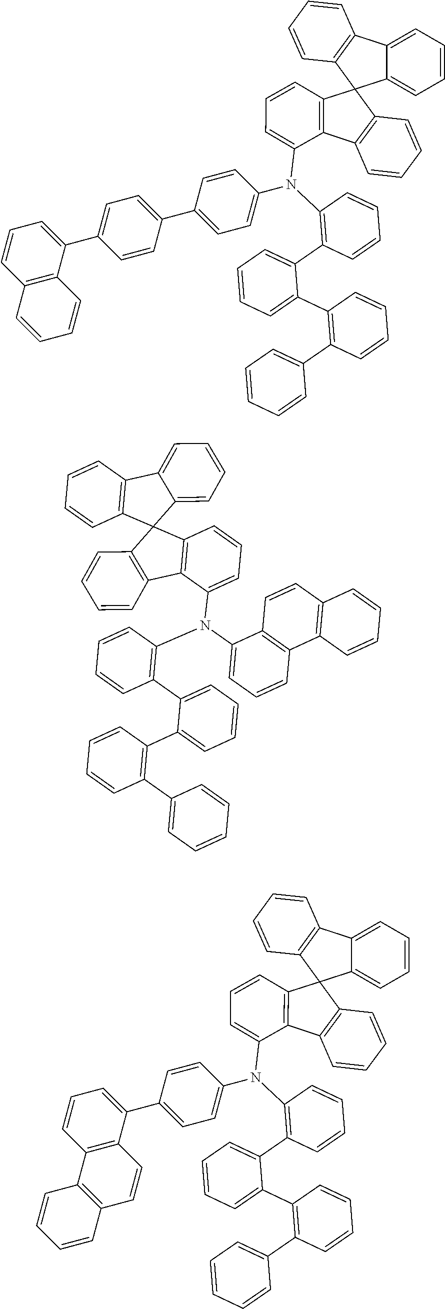 [SMILES: c1ccc(-c2ccccc2-c2ccccc2-c2ccccc2N(c2ccc(-c3ccc(-c4cccc5ccccc45)cc3)cc2)c2cccc3c2-c2ccccc2C32c3ccccc3-c3ccccc32)cc1 ; c1ccc(-c2ccccc2-c2ccccc2-c2ccccc2N(c2cccc3c2-c2ccccc2C32c3ccccc3-c3ccccc32)c2cccc3c2ccc2ccccc23)cc1 ; c1ccc(-c2ccccc2-c2ccccc2-c2ccccc2N(c2ccc(-c3cccc4c3ccc3ccccc34)cc2)c2cccc3c2-c2ccccc2C32c3ccccc3-c3ccccc32)cc1]

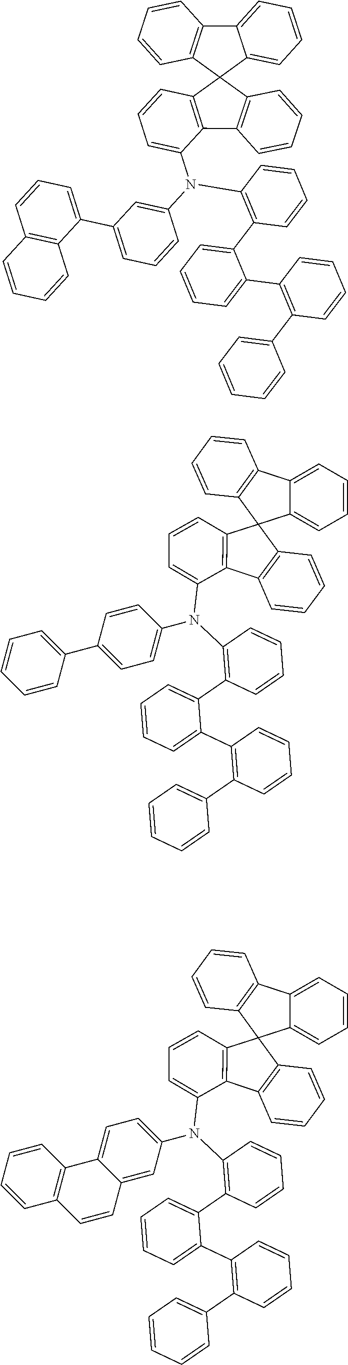

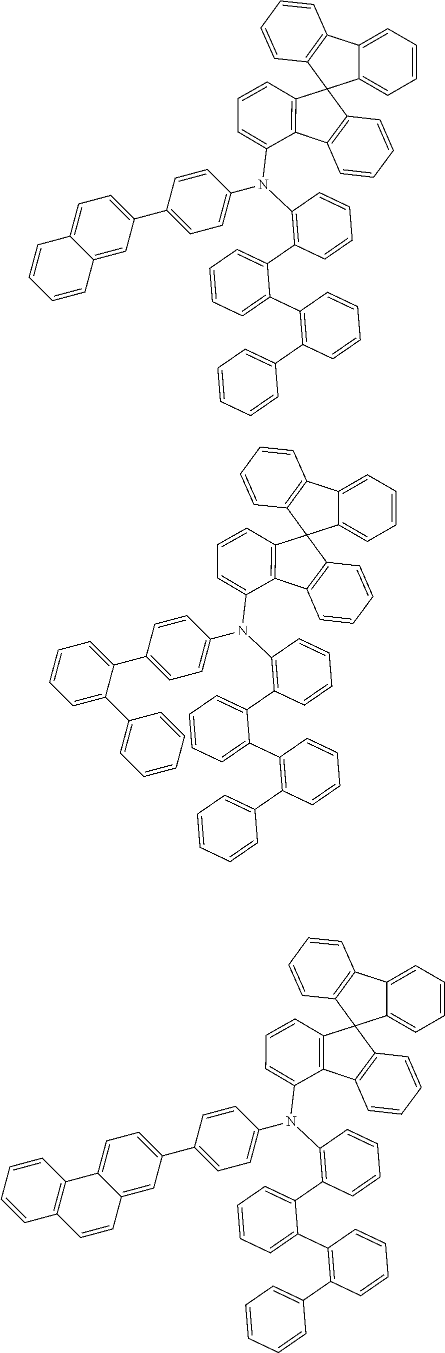

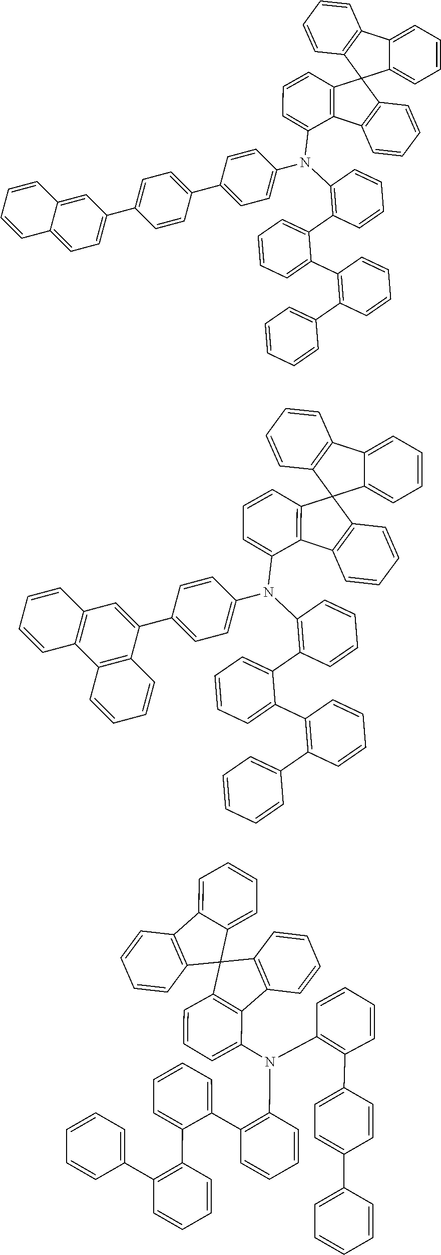

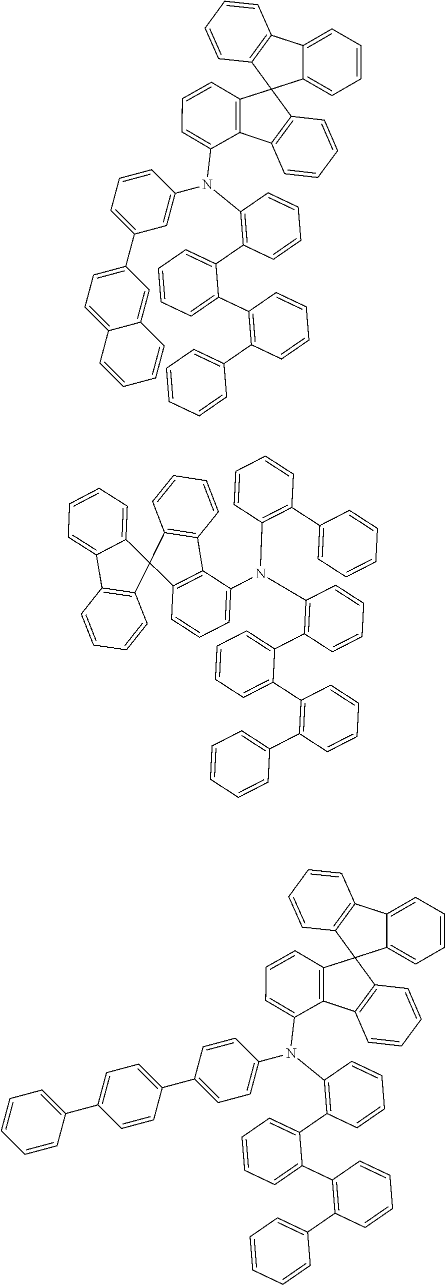 [SMILES: c1ccc(-c2ccccc2-c2ccccc2-c2ccccc2N(c2cccc(-c3ccc4ccccc4c3)c2)c2cccc3c2-c2ccccc2C32c3ccccc3-c3ccccc32)cc1 ; c1ccc(-c2ccccc2-c2ccccc2-c2ccccc2N(c2ccccc2-c2ccccc2)c2cccc3c2-c2ccccc2C32c3ccccc3-c3ccccc32)cc1 ; c1ccc(-c2ccc(-c3ccc(N(c4ccccc4-c4ccccc4-c4ccccc4-c4ccccc4)c4cccc5c4-c4ccccc4C54c5ccccc5-c5ccccc54)cc3)cc2)cc1]

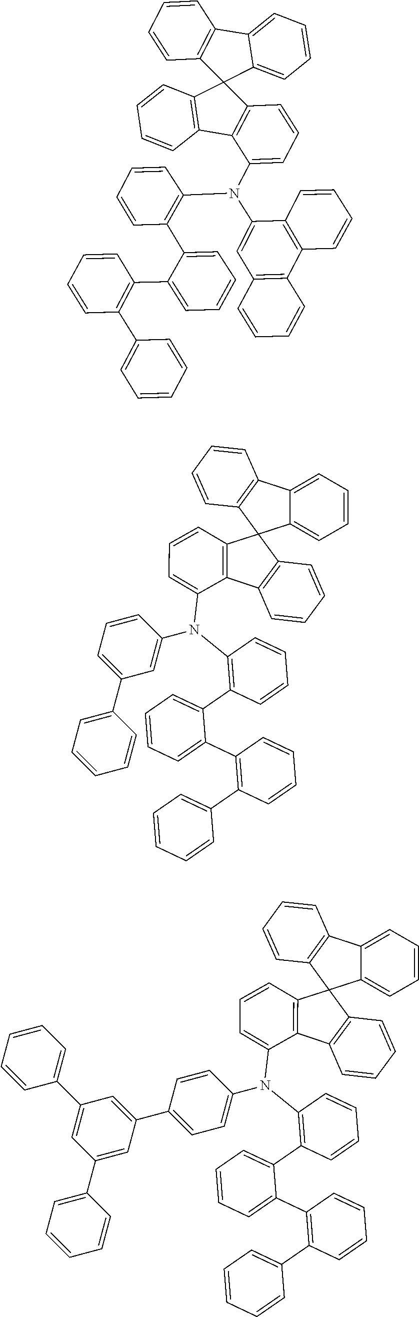 [SMILES: c1ccc(-c2ccccc2-c2ccccc2-c2ccccc2N(c2cccc3c2-c2ccccc2C32c3ccccc3-c3ccccc32)c2cc3ccccc3c3ccccc23)cc1 ; c1ccc(-c2cccc(N(c3ccccc3-c3ccccc3-c3ccccc3-c3ccccc3)c3cccc4c3-c3ccccc3C43c4ccccc4-c4ccccc43)c2)cc1 ; c1ccc(-c2cc(-c3ccccc3)cc(-c3ccc(N(c4ccccc4-c4ccccc4-c4ccccc4-c4ccccc4)c4cccc5c4-c4ccccc4C54c5ccccc5-c5ccccc54)cc3)c2)cc1]

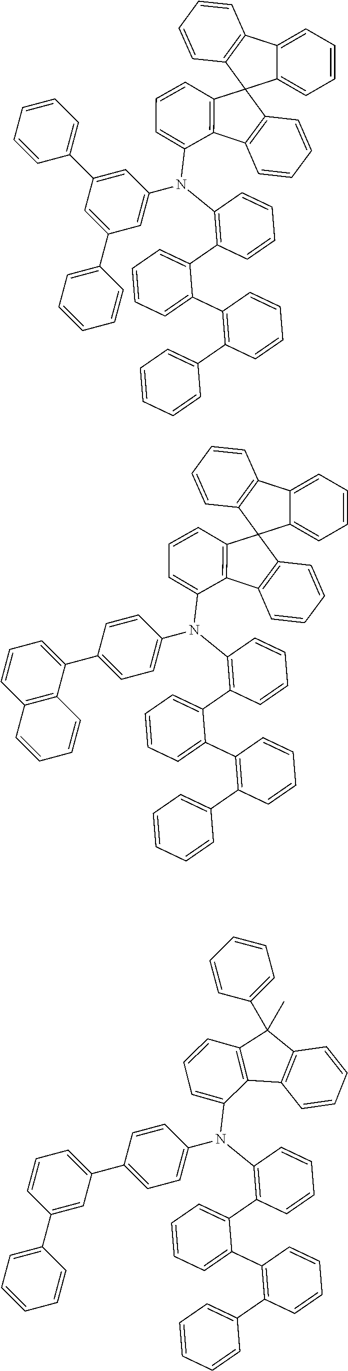 [SMILES: c1ccc(-c2cc(-c3ccccc3)cc(N(c3ccccc3-c3ccccc3-c3ccccc3-c3ccccc3)c3cccc4c3-c3ccccc3C43c4ccccc4-c4ccccc43)c2)cc1 ; c1ccc(-c2ccccc2-c2ccccc2-c2ccccc2N(c2ccc(-c3cccc4ccccc34)cc2)c2cccc3c2-c2ccccc2C32c3ccccc3-c3ccccc32)cc1 ; CC1(c2ccccc2)c2ccccc2-c2c(N(c3ccc(-c4cccc(-c5ccccc5)c4)cc3)c3ccccc3-c3ccccc3-c3ccccc3-c3ccccc3)cccc21]

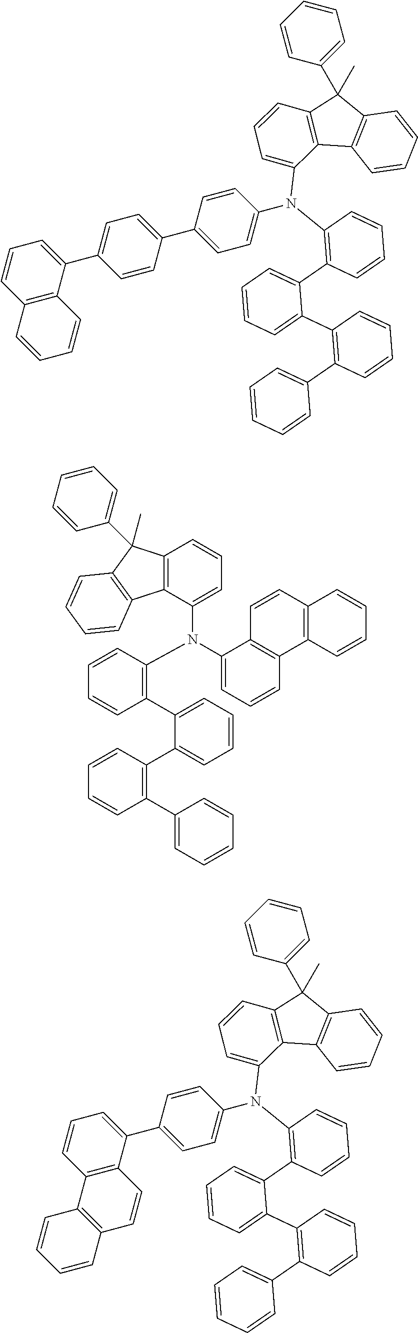 [SMILES: CC1(c2ccccc2)c2ccccc2-c2c(N(c3ccc(-c4ccc(-c5cccc6ccccc56)cc4)cc3)c3ccccc3-c3ccccc3-c3ccccc3-c3ccccc3)cccc21 ; CC1(c2ccccc2)c2ccccc2-c2c(N(c3ccccc3-c3ccccc3-c3ccccc3-c3ccccc3)c3cccc4c3ccc3ccccc34)cccc21 ; CC1(c2ccccc2)c2ccccc2-c2c(N(c3ccc(-c4cccc5c4ccc4ccccc45)cc3)c3ccccc3-c3ccccc3-c3ccccc3-c3ccccc3)cccc21]

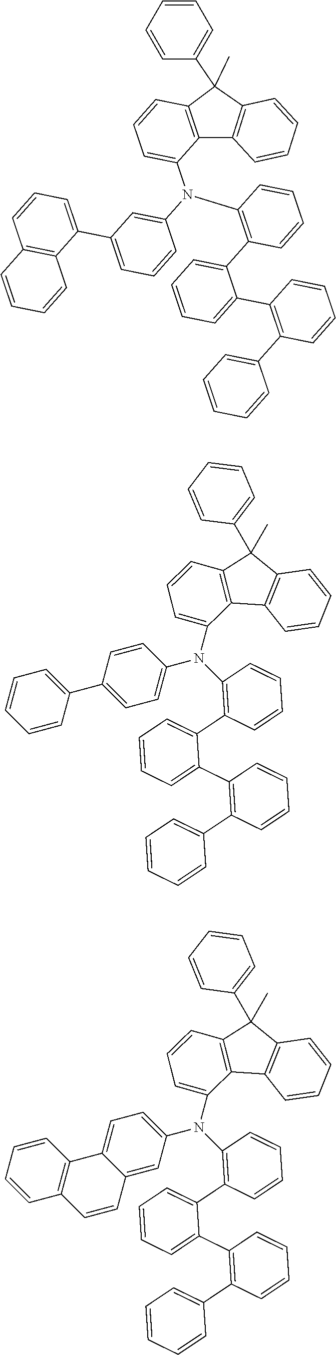 [SMILES: CC1(c2ccccc2)c2ccccc2-c2c(N(c3cccc(-c4cccc5ccccc45)c3)c3ccccc3-c3ccccc3-c3ccccc3-c3ccccc3)cccc21 ; CC1(c2ccccc2)c2ccccc2-c2c(N(c3ccc(-c4ccccc4)cc3)c3ccccc3-c3ccccc3-c3ccccc3-c3ccccc3)cccc21 ; CC1(c2ccccc2)c2ccccc2-c2c(N(c3ccc4c(ccc5ccccc54)c3)c3ccccc3-c3ccccc3-c3ccccc3-c3ccccc3)cccc21]

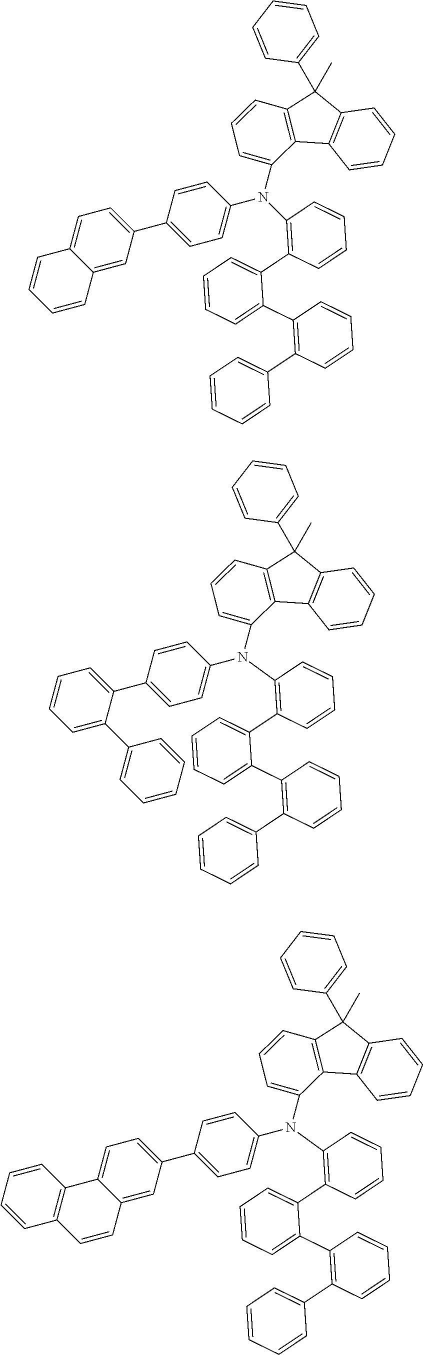

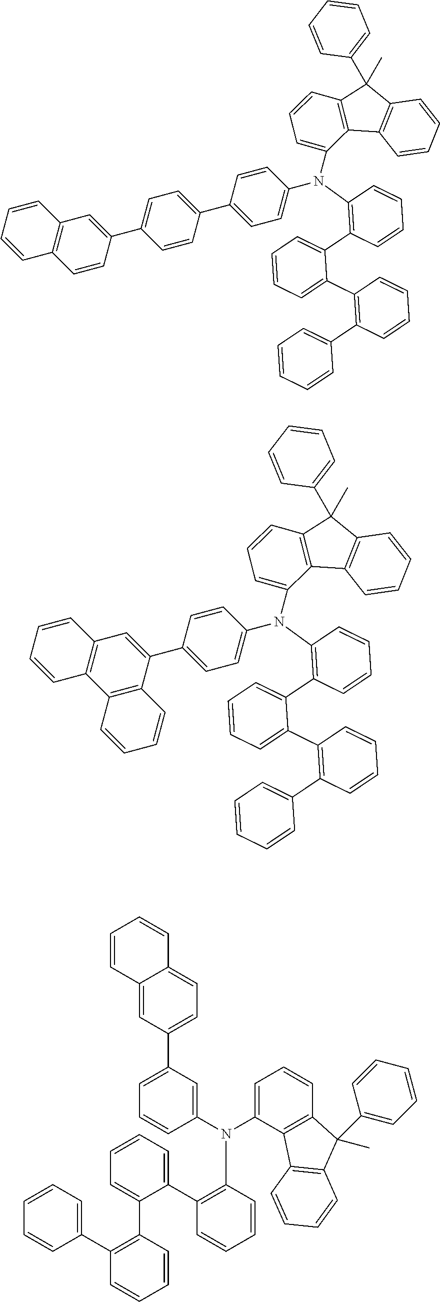

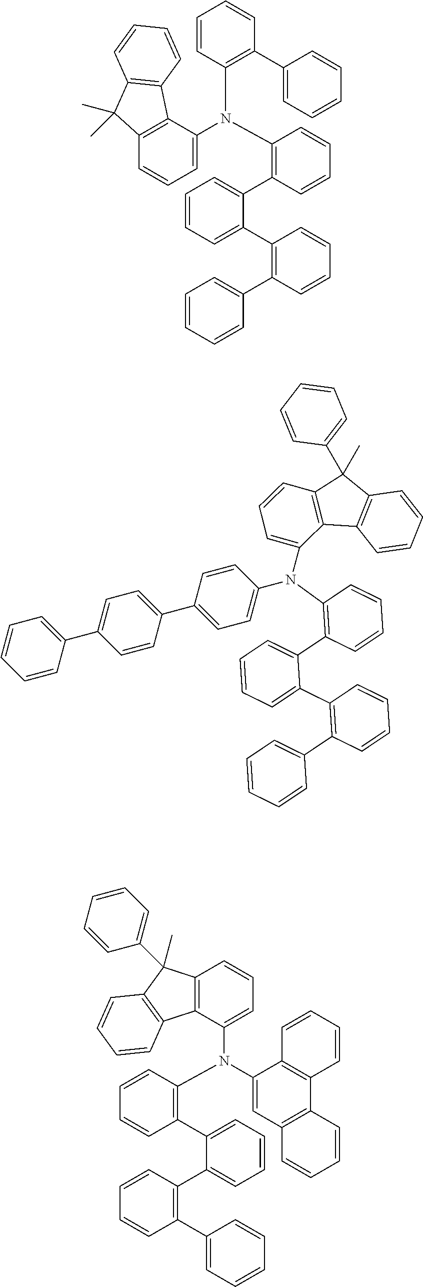 [SMILES: CC1(C)c2ccccc2-c2c(N(c3ccccc3-c3ccccc3)c3ccccc3-c3ccccc3-c3ccccc3-c3ccccc3)cccc21 ; CC1(c2ccccc2)c2ccccc2-c2c(N(c3ccc(-c4ccc(-c5ccccc5)cc4)cc3)c3ccccc3-c3ccccc3-c3ccccc3-c3ccccc3)cccc21 ; CC1(c2ccccc2)c2ccccc2-c2c(N(c3ccccc3-c3ccccc3-c3ccccc3-c3ccccc3)c3cc4ccccc4c4ccccc34)cccc21]

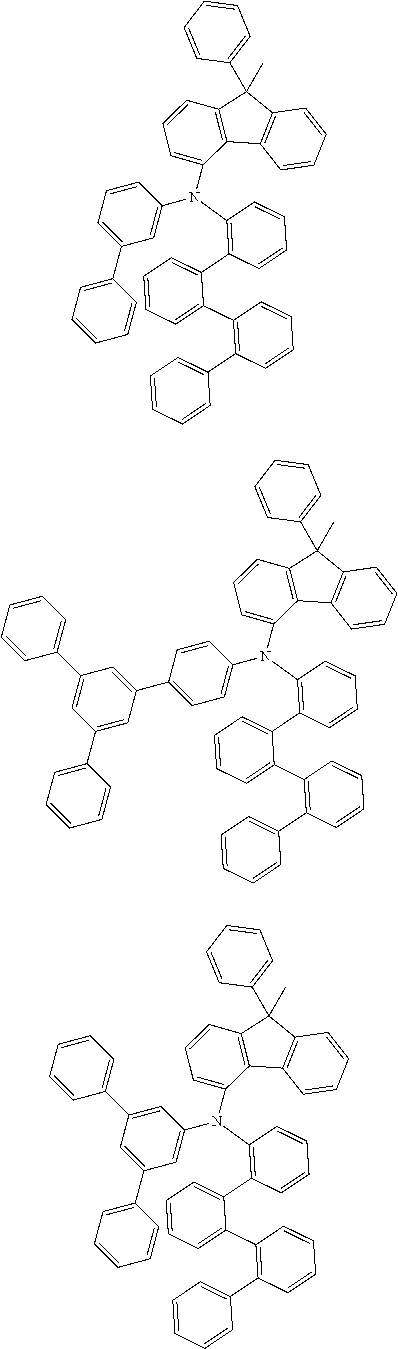 [SMILES: CC1(c2ccccc2)c2ccccc2-c2c(N(c3cccc(-c4ccccc4)c3)c3ccccc3-c3ccccc3-c3ccccc3-c3ccccc3)cccc21 ; CC1(c2ccccc2)c2ccccc2-c2c(N(c3ccc(-c4cc(-c5ccccc5)cc(-c5ccccc5)c4)cc3)c3ccccc3-c3ccccc3-c3ccccc3-c3ccccc3)cccc21 ; CC1(c2ccccc2)c2ccccc2-c2c(N(c3cc(-c4ccccc4)cc(-c4ccccc4)c3)c3ccccc3-c3ccccc3-c3ccccc3-c3ccccc3)cccc21]

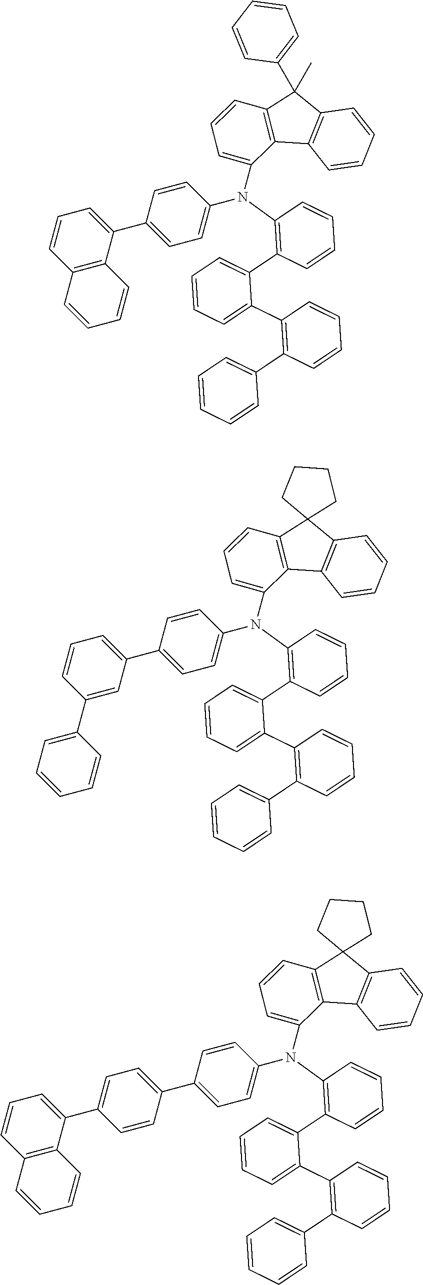 [SMILES: CC1(c2ccccc2)c2ccccc2-c2c(N(c3ccc(-c4cccc5ccccc45)cc3)c3ccccc3-c3ccccc3-c3ccccc3-c3ccccc3)cccc21 ; c1ccc(-c2cccc(-c3ccc(N(c4ccccc4-c4ccccc4-c4ccccc4-c4ccccc4)c4cccc5c4-c4ccccc4C54CCCC4)cc3)c2)cc1 ; c1ccc(-c2ccccc2-c2ccccc2-c2ccccc2N(c2ccc(-c3ccc(-c4cccc5ccccc45)cc3)cc2)c2cccc3c2-c2ccccc2C32CCCC2)cc1]

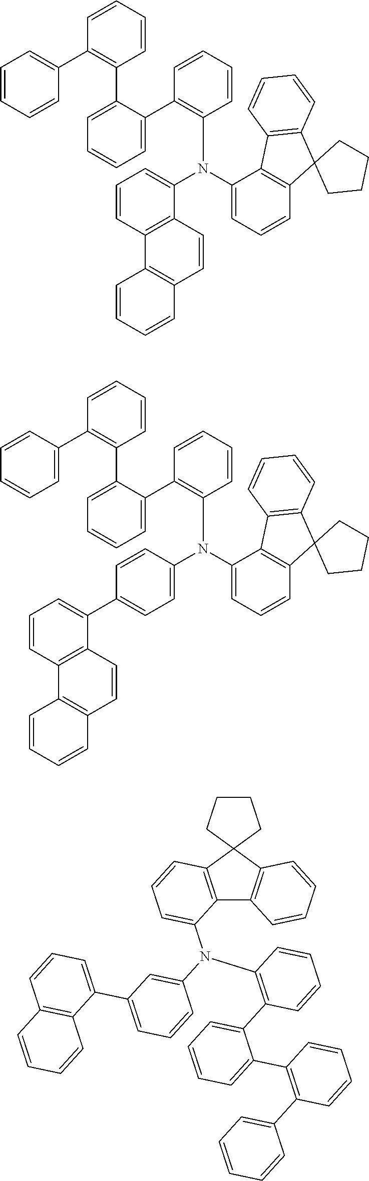 [SMILES: c1ccc(-c2ccccc2-c2ccccc2-c2ccccc2N(c2cccc3c2-c2ccccc2C32CCCC2)c2cccc3c2ccc2ccccc23)cc1 ; c1ccc(-c2ccccc2-c2ccccc2-c2ccccc2N(c2ccc(-c3cccc4c3ccc3ccccc34)cc2)c2cccc3c2-c2ccccc2C32CCCC2)cc1 ; c1ccc(-c2ccccc2-c2ccccc2-c2ccccc2N(c2cccc(-c3cccc4ccccc34)c2)c2cccc3c2-c2ccccc2C32CCCC2)cc1]

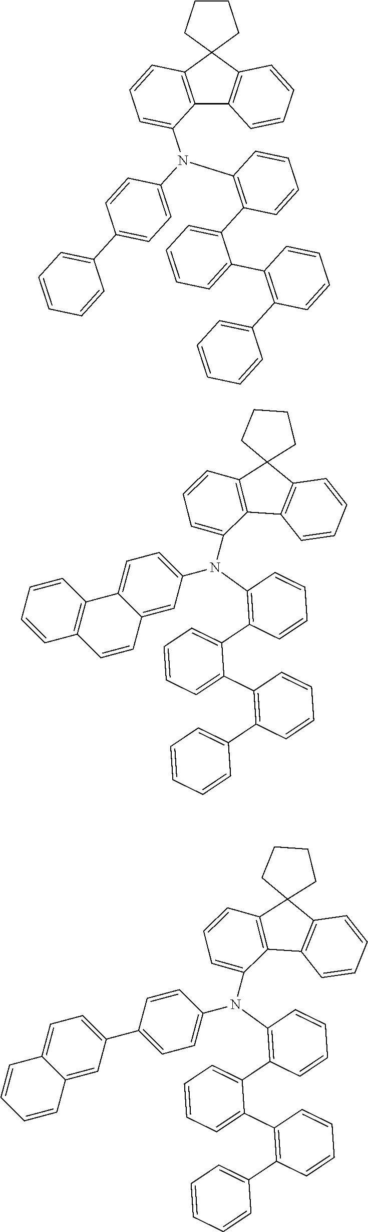

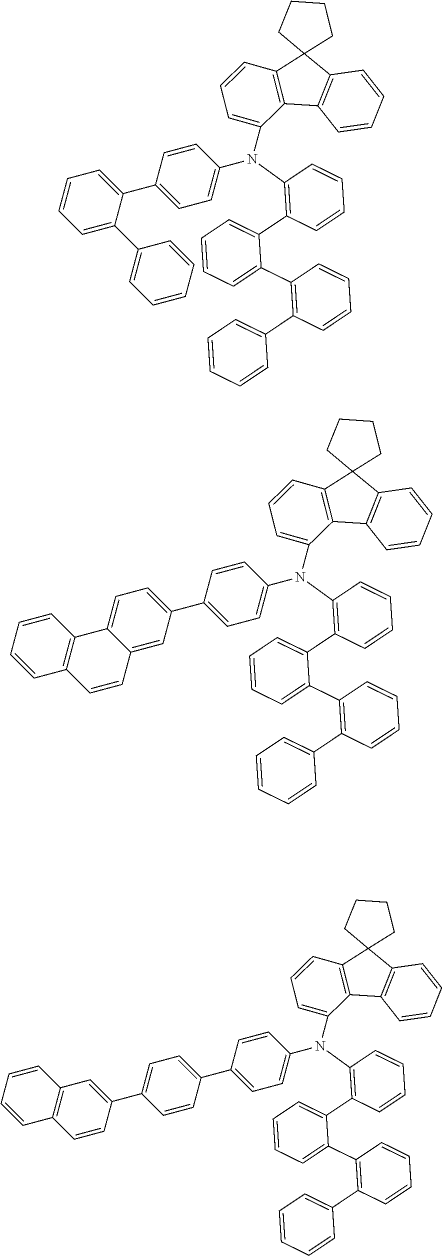 [SMILES: c1ccc(-c2ccccc2-c2ccc(N(c3ccccc3-c3ccccc3-c3ccccc3-c3ccccc3)c3cccc4c3-c3ccccc3C43CCCC3)cc2)cc1 ; c1ccc(-c2ccccc2-c2ccccc2-c2ccccc2N(c2ccc(-c3ccc4c(ccc5ccccc54)c3)cc2)c2cccc3c2-c2ccccc2C32CCCC2)cc1 ; c1ccc(-c2ccccc2-c2ccccc2-c2ccccc2N(c2ccc(-c3ccc(-c4ccc5ccccc5c4)cc3)cc2)c2cccc3c2-c2ccccc2C32CCCC2)cc1]

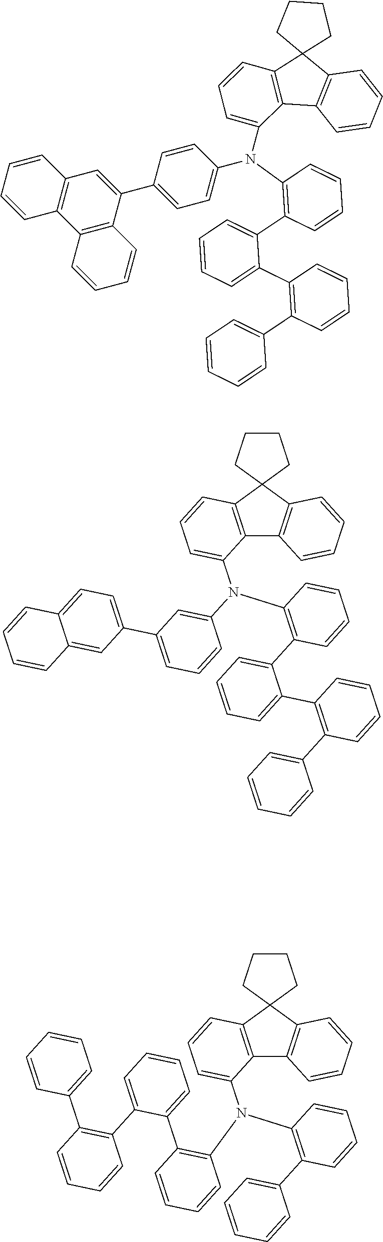 [SMILES: c1ccc(-c2ccccc2-c2ccccc2-c2ccccc2N(c2ccc(-c3cc4ccccc4c4ccccc34)cc2)c2cccc3c2-c2ccccc2C32CCCC2)cc1 ; c1ccc(-c2ccccc2-c2ccccc2-c2ccccc2N(c2cccc(-c3ccc4ccccc4c3)c2)c2cccc3c2-c2ccccc2C32CCCC2)cc1 ; c1ccc(-c2ccccc2-c2ccccc2-c2ccccc2N(c2ccccc2-c2ccccc2)c2cccc3c2-c2ccccc2C32CCCC2)cc1]

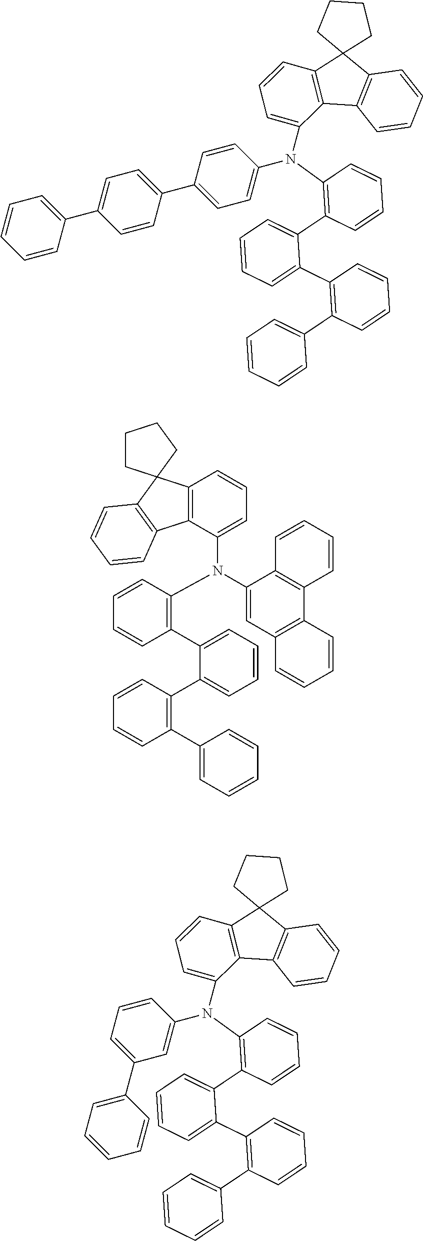 [SMILES: c1ccc(-c2ccc(-c3ccc(N(c4ccccc4-c4ccccc4-c4ccccc4-c4ccccc4)c4cccc5c4-c4ccccc4C54CCCC4)cc3)cc2)cc1 ; c1ccc(-c2ccccc2-c2ccccc2-c2ccccc2N(c2cccc3c2-c2ccccc2C32CCCC2)c2cc3ccccc3c3ccccc23)cc1 ; c1ccc(-c2cccc(N(c3ccccc3-c3ccccc3-c3ccccc3-c3ccccc3)c3cccc4c3-c3ccccc3C43CCCC3)c2)cc1]

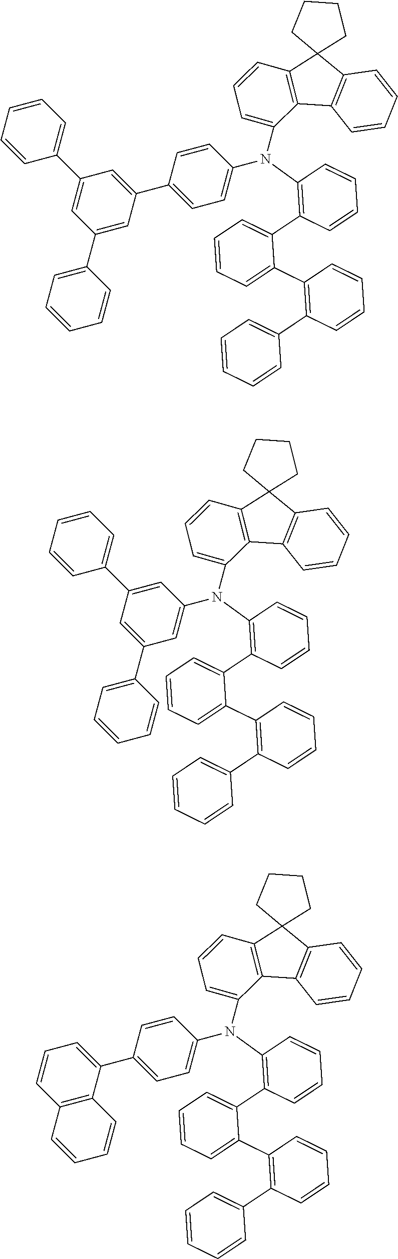 [SMILES: c1ccc(-c2cc(-c3ccccc3)cc(-c3ccc(N(c4ccccc4-c4ccccc4-c4ccccc4-c4ccccc4)c4cccc5c4-c4ccccc4C54CCCC4)cc3)c2)cc1 ; c1ccc(-c2cc(-c3ccccc3)cc(N(c3ccccc3-c3ccccc3-c3ccccc3-c3ccccc3)c3cccc4c3-c3ccccc3C43CCCC3)c2)cc1 ; c1ccc(-c2ccccc2-c2ccccc2-c2ccccc2N(c2ccc(-c3cccc4ccccc34)cc2)c2cccc3c2-c2ccccc2C32CCCC2)cc1]

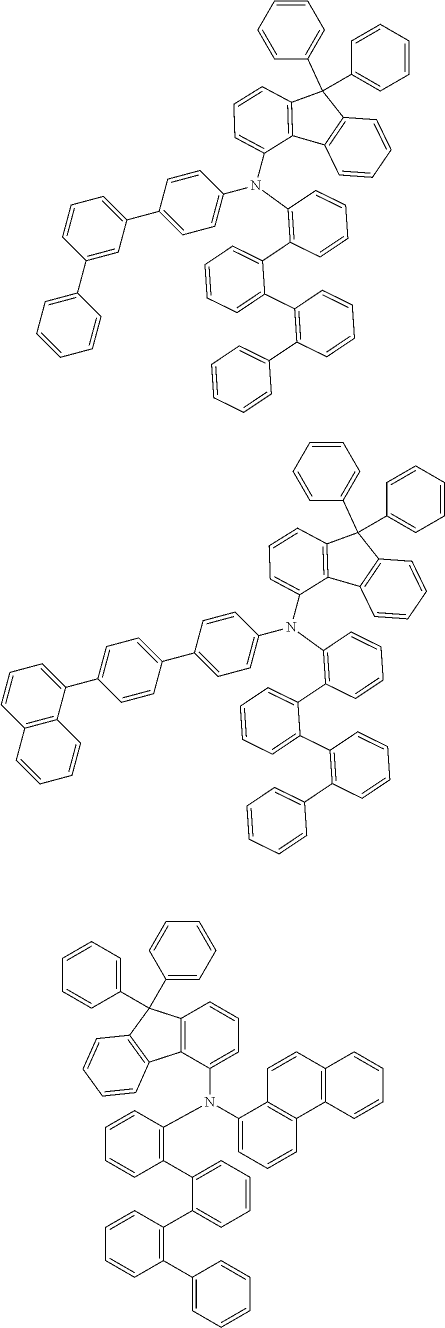 [SMILES: c1ccc(-c2cccc(-c3ccc(N(c4ccccc4-c4ccccc4-c4ccccc4-c4ccccc4)c4cccc5c4-c4ccccc4C5(c4ccccc4)c4ccccc4)cc3)c2)cc1 ; c1ccc(-c2ccccc2-c2ccccc2-c2ccccc2N(c2ccc(-c3ccc(-c4cccc5ccccc45)cc3)cc2)c2cccc3c2-c2ccccc2C3(c2ccccc2)c2ccccc2)cc1 ; c1ccc(-c2ccccc2-c2ccccc2-c2ccccc2N(c2cccc3c2-c2ccccc2C3(c2ccccc2)c2ccccc2)c2cccc3c2ccc2ccccc23)cc1]

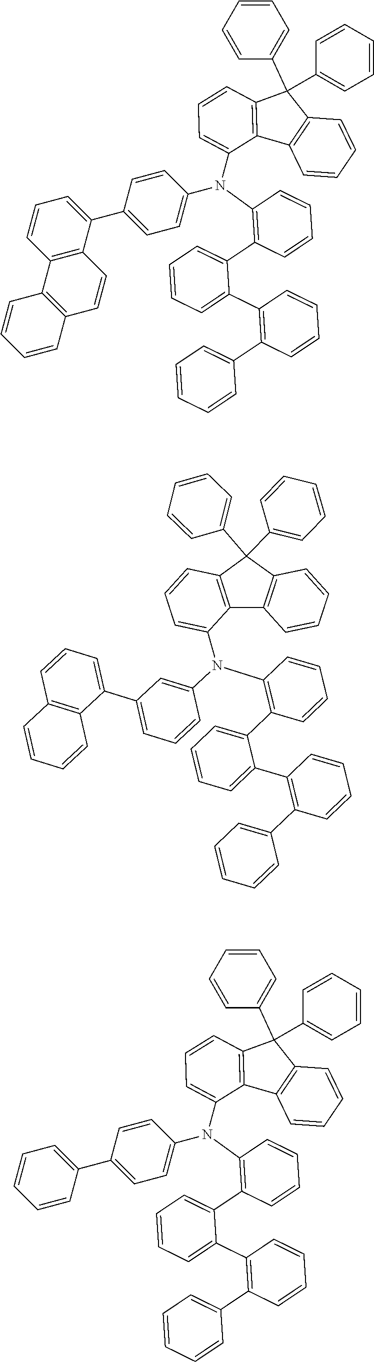 [SMILES: c1ccc(-c2ccccc2-c2ccccc2-c2ccccc2N(c2ccc(-c3cccc4c3ccc3ccccc34)cc2)c2cccc3c2-c2ccccc2C3(c2ccccc2)c2ccccc2)cc1 ; c1ccc(-c2ccccc2-c2ccccc2-c2ccccc2N(c2cccc(-c3cccc4ccccc34)c2)c2cccc3c2-c2ccccc2C3(c2ccccc2)c2ccccc2)cc1 ; c1ccc(-c2ccc(N(c3ccccc3-c3ccccc3-c3ccccc3-c3ccccc3)c3cccc4c3-c3ccccc3C4(c3ccccc3)c3ccccc3)cc2)cc1]

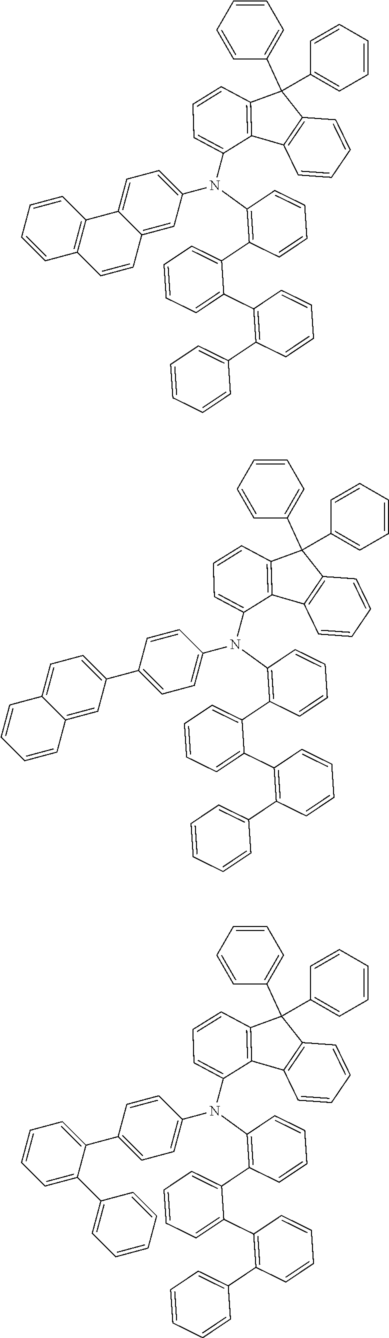 [SMILES: c1ccc(-c2ccccc2-c2ccccc2-c2ccccc2N(c2ccc3c(ccc4ccccc43)c2)c2cccc3c2-c2ccccc2C3(c2ccccc2)c2ccccc2)cc1 ; c1ccc(-c2ccccc2-c2ccccc2-c2ccccc2N(c2ccc(-c3ccc4ccccc4c3)cc2)c2cccc3c2-c2ccccc2C3(c2ccccc2)c2ccccc2)cc1 ; c1ccc(-c2ccccc2-c2ccc(N(c3ccccc3-c3ccccc3-c3ccccc3-c3ccccc3)c3cccc4c3-c3ccccc3C4(c3ccccc3)c3ccccc3)cc2)cc1]

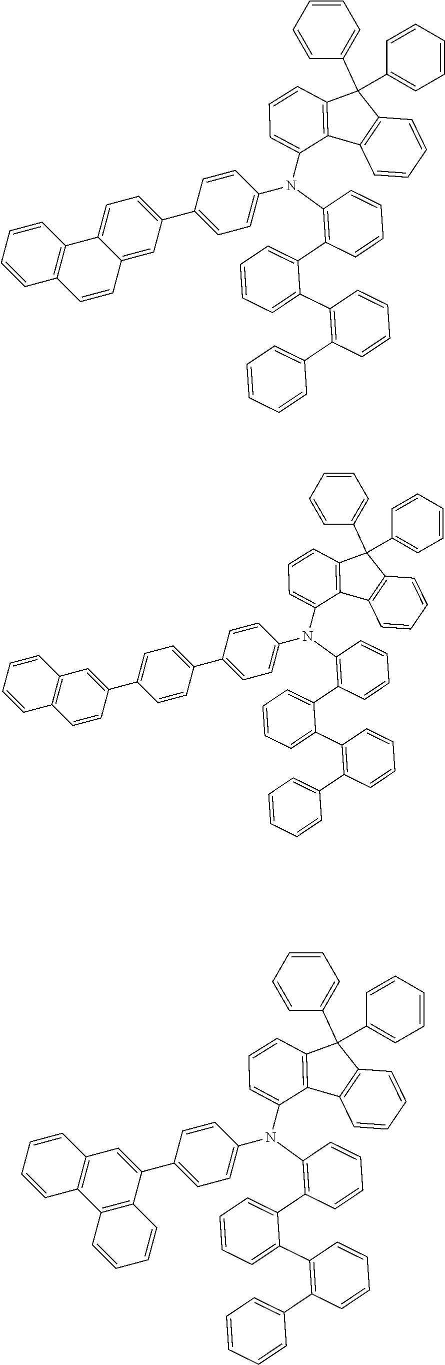 [SMILES: c1ccc(-c2ccccc2-c2ccccc2-c2ccccc2N(c2ccc(-c3ccc4c(ccc5ccccc54)c3)cc2)c2cccc3c2-c2ccccc2C3(c2ccccc2)c2ccccc2)cc1 ; c1ccc(-c2ccccc2-c2ccccc2-c2ccccc2N(c2ccc(-c3ccc(-c4ccc5ccccc5c4)cc3)cc2)c2cccc3c2-c2ccccc2C3(c2ccccc2)c2ccccc2)cc1 ; c1ccc(-c2ccccc2-c2ccccc2-c2ccccc2N(c2ccc(-c3cc4ccccc4c4ccccc34)cc2)c2cccc3c2-c2ccccc2C3(c2ccccc2)c2ccccc2)cc1]

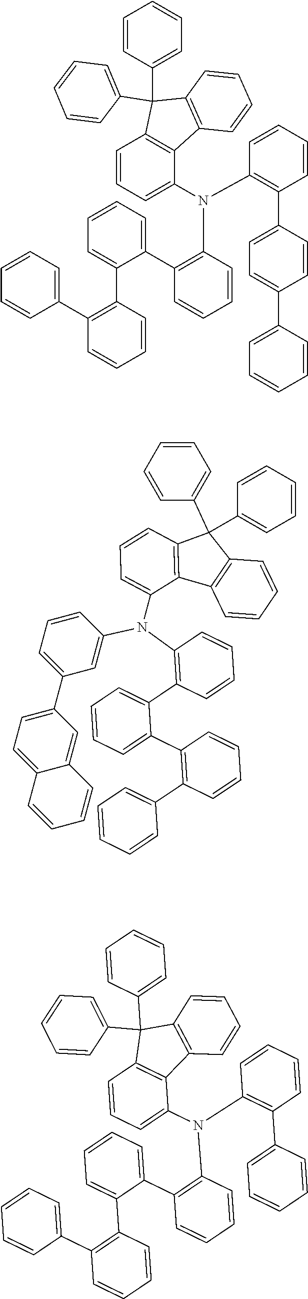 [SMILES: c1ccc(-c2ccc(-c3ccccc3N(c3ccccc3-c3ccccc3-c3ccccc3-c3ccccc3)c3cccc4c3-c3ccccc3C4(c3ccccc3)c3ccccc3)cc2)cc1 ; c1ccc(-c2ccccc2-c2ccccc2-c2ccccc2N(c2cccc(-c3ccc4ccccc4c3)c2)c2cccc3c2-c2ccccc2C3(c2ccccc2)c2ccccc2)cc1 ; c1ccc(-c2ccccc2-c2ccccc2-c2ccccc2N(c2ccccc2-c2ccccc2)c2cccc3c2-c2ccccc2C3(c2ccccc2)c2ccccc2)cc1]

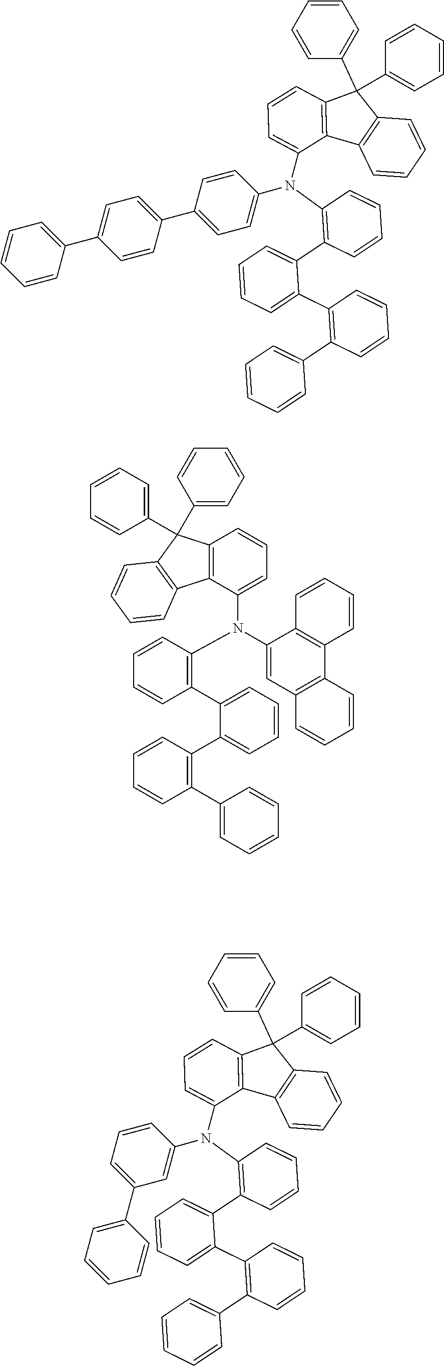 [SMILES: c1ccc(-c2ccc(-c3ccc(N(c4ccccc4-c4ccccc4-c4ccccc4-c4ccccc4)c4cccc5c4-c4ccccc4C5(c4ccccc4)c4ccccc4)cc3)cc2)cc1 ; c1ccc(-c2ccccc2-c2ccccc2-c2ccccc2N(c2cccc3c2-c2ccccc2C3(c2ccccc2)c2ccccc2)c2cc3ccccc3c3ccccc23)cc1 ; c1ccc(-c2cccc(N(c3ccccc3-c3ccccc3-c3ccccc3-c3ccccc3)c3cccc4c3-c3ccccc3C4(c3ccccc3)c3ccccc3)c2)cc1]

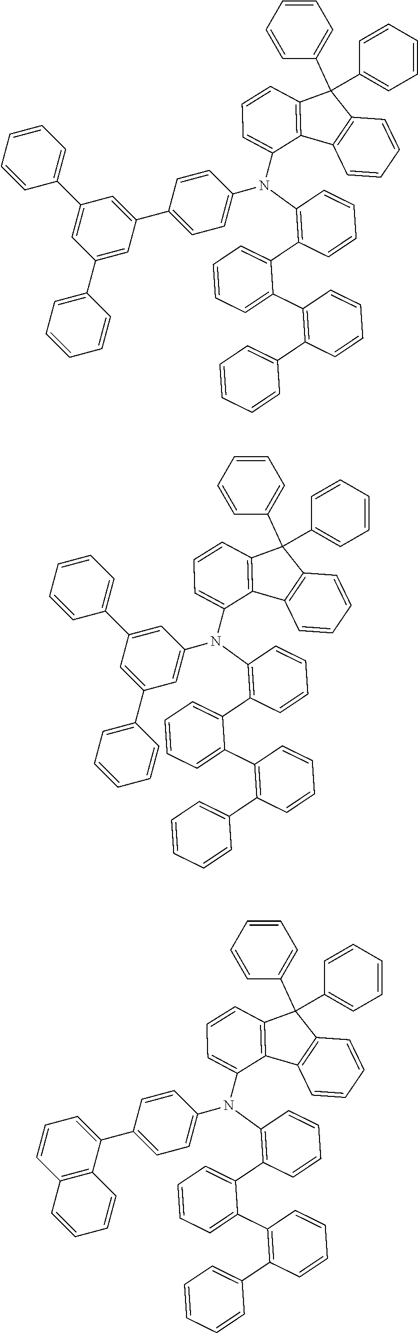 [SMILES: c1ccc(-c2cc(-c3ccccc3)cc(-c3ccc(N(c4ccccc4-c4ccccc4-c4ccccc4-c4ccccc4)c4cccc5c4-c4ccccc4C5(c4ccccc4)c4ccccc4)cc3)c2)cc1 ; c1ccc(-c2cc(-c3ccccc3)cc(N(c3ccccc3-c3ccccc3-c3ccccc3-c3ccccc3)c3cccc4c3-c3ccccc3C4(c3ccccc3)c3ccccc3)c2)cc1 ; c1ccc(-c2ccccc2-c2ccccc2-c2ccccc2N(c2ccc(-c3cccc4ccccc34)cc2)c2cccc3c2-c2ccccc2C3(c2ccccc2)c2ccccc2)cc1]

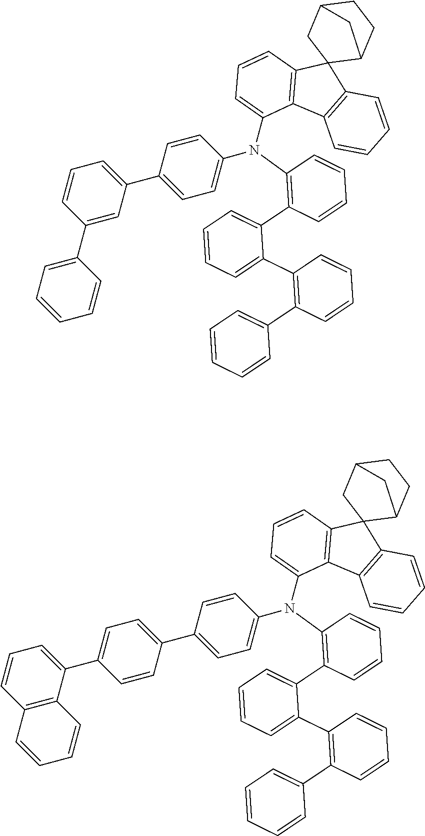 [SMILES: c1ccc(-c2cccc(-c3ccc(N(c4ccccc4-c4ccccc4-c4ccccc4-c4ccccc4)c4cccc5c4-c4ccccc4C54CC5CCC4C5)cc3)c2)cc1 ; c1ccc(-c2ccccc2-c2ccccc2-c2ccccc2N(c2ccc(-c3ccc(-c4cccc5ccccc45)cc3)cc2)c2cccc3c2-c2ccccc2C32CC3CCC2C3)cc1]

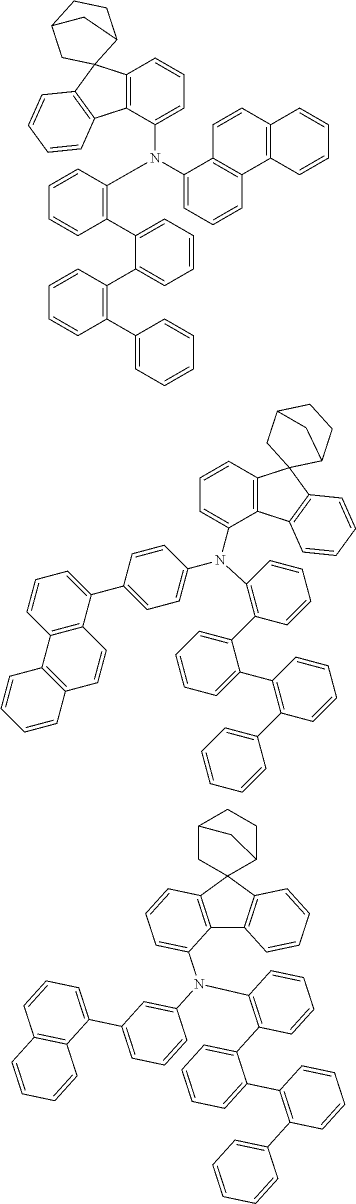

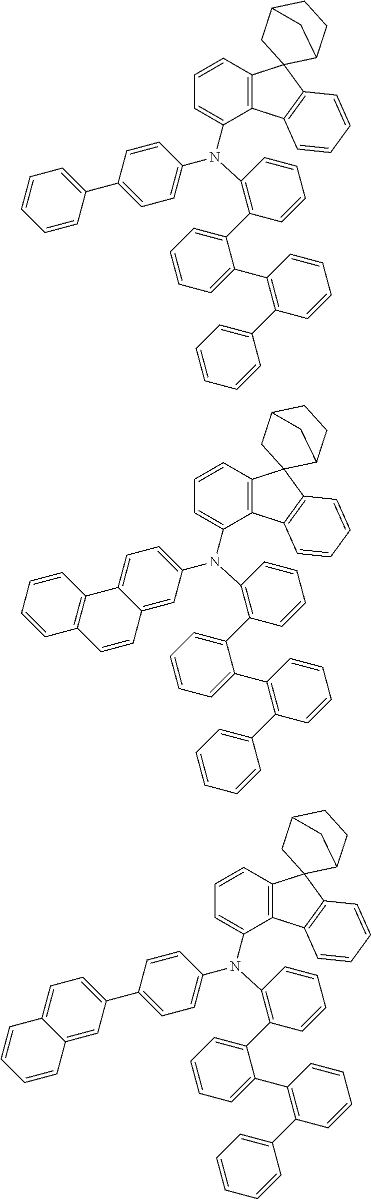

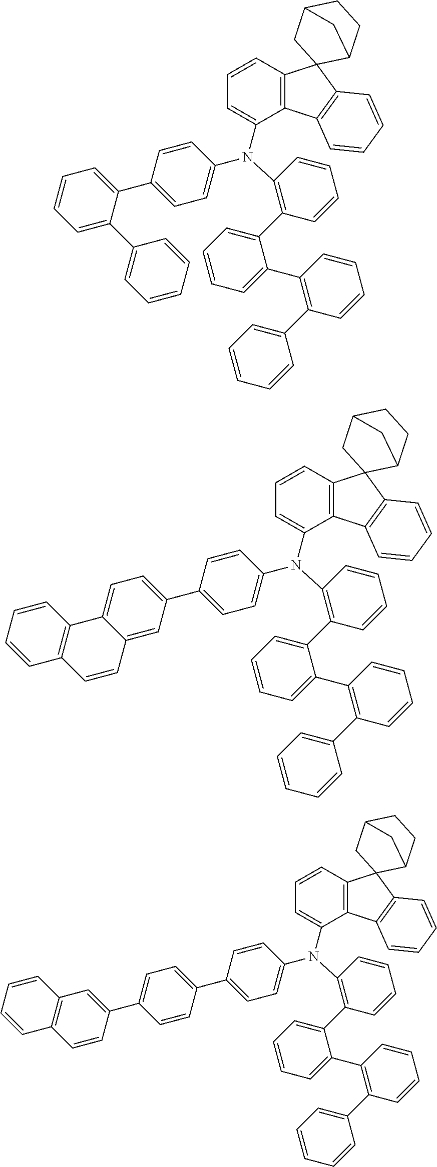 [SMILES: c1ccc(-c2ccccc2-c2ccc(N(c3ccccc3-c3ccccc3-c3ccccc3-c3ccccc3)c3cccc4c3-c3ccccc3C43CC4CCC3C4)cc2)cc1 ; c1ccc(-c2ccccc2-c2ccccc2-c2ccccc2N(c2ccc(-c3ccc4c(ccc5ccccc54)c3)cc2)c2cccc3c2-c2ccccc2C32CC3CCC2C3)cc1 ; c1ccc(-c2ccccc2-c2ccccc2-c2ccccc2N(c2ccc(-c3ccc(-c4ccc5ccccc5c4)cc3)cc2)c2cccc3c2-c2ccccc2C32CC3CCC2C3)cc1]

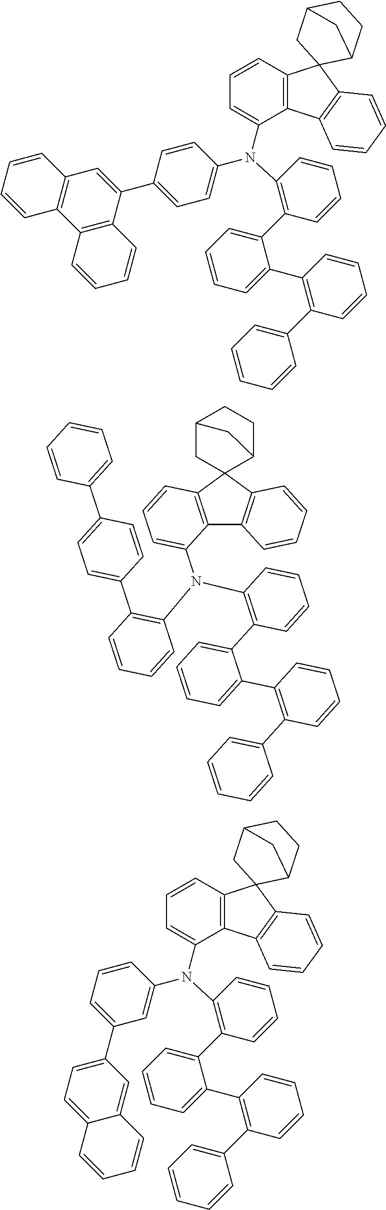

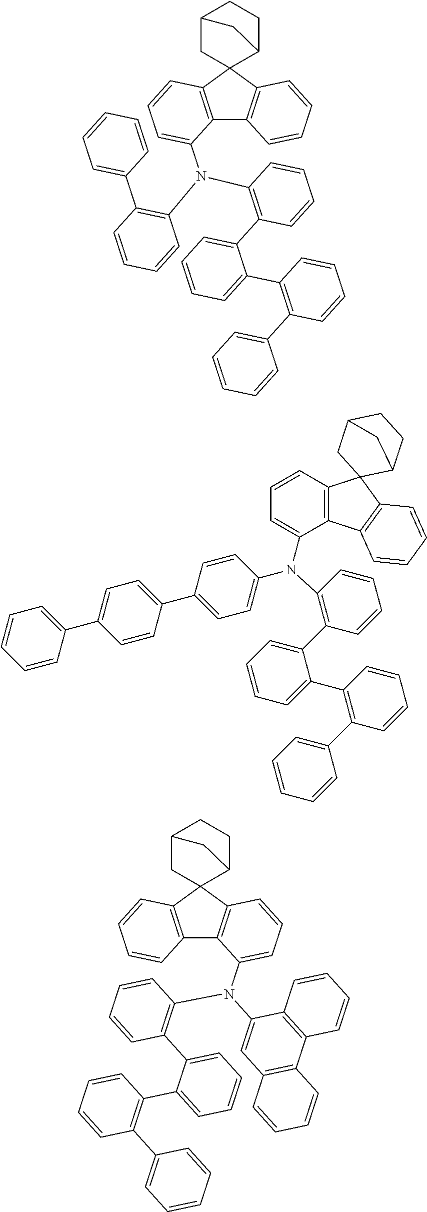 [SMILES: c1ccc(-c2ccccc2-c2ccccc2-c2ccccc2N(c2ccccc2-c2ccccc2)c2cccc3c2-c2ccccc2C32CC3CCC2C3)cc1 ; c1ccc(-c2ccc(-c3ccc(N(c4ccccc4-c4ccccc4-c4ccccc4-c4ccccc4)c4cccc5c4-c4ccccc4C54CC5CCC4C5)cc3)cc2)cc1 ; c1ccc(-c2ccccc2-c2ccccc2-c2ccccc2N(c2cccc3c2-c2ccccc2C32CC3CCC2C3)c2cc3ccccc3c3ccccc23)cc1]

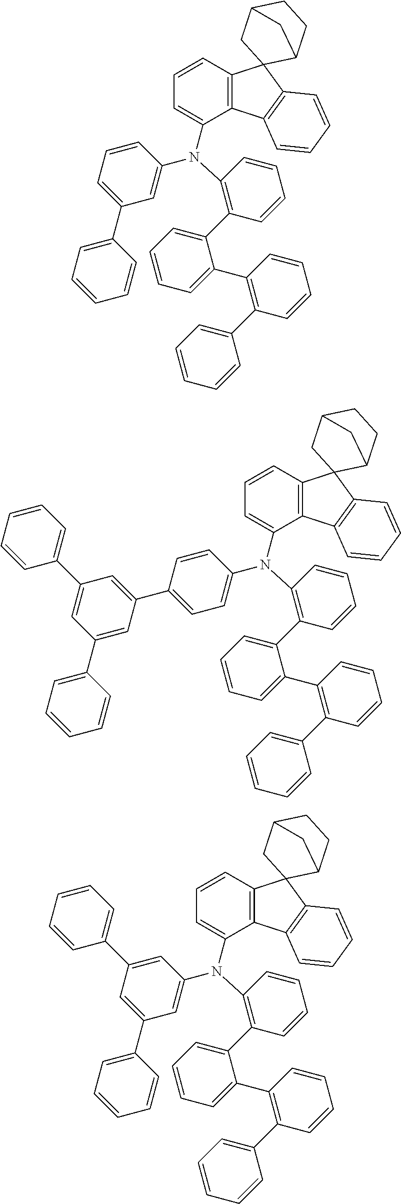

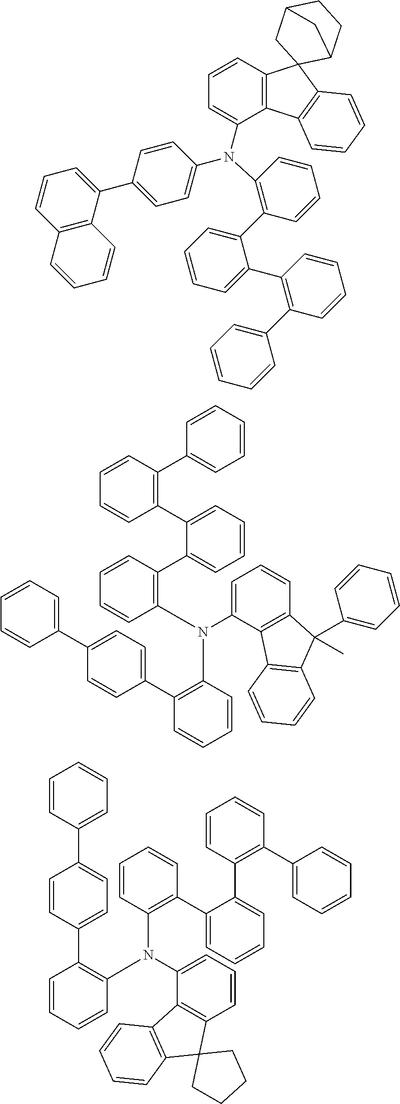

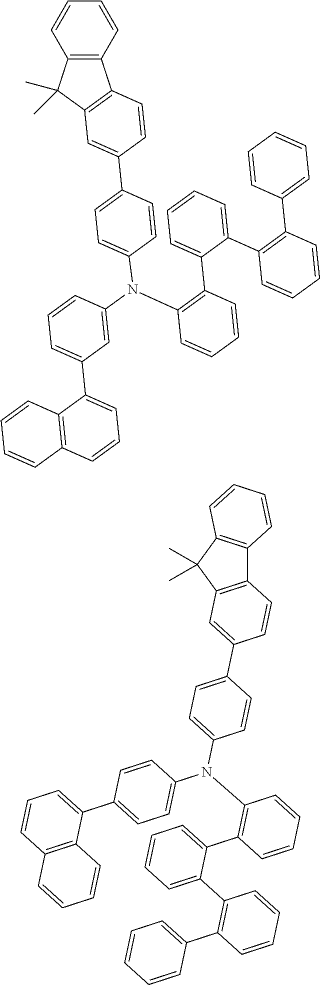 [SMILES: CC1(C)c2ccccc2-c2ccc(-c3ccc(N(c4cccc(-c5cccc6ccccc56)c4)c4ccccc4-c4ccccc4-c4ccccc4-c4ccccc4)cc3)cc21 ; CC1(C)c2ccccc2-c2ccc(-c3ccc(N(c4ccc(-c5cccc6ccccc56)cc4)c4ccccc4-c4ccccc4-c4ccccc4-c4ccccc4)cc3)cc21]

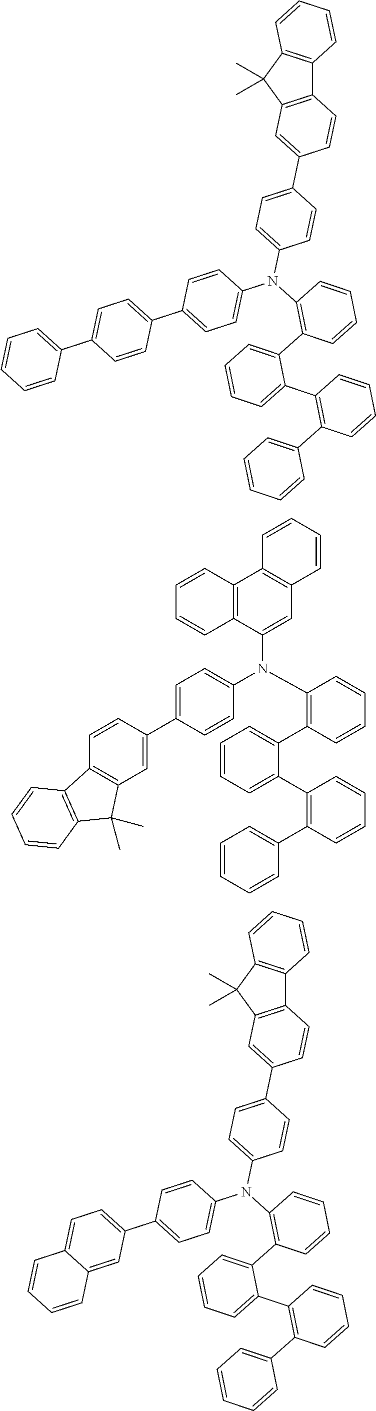

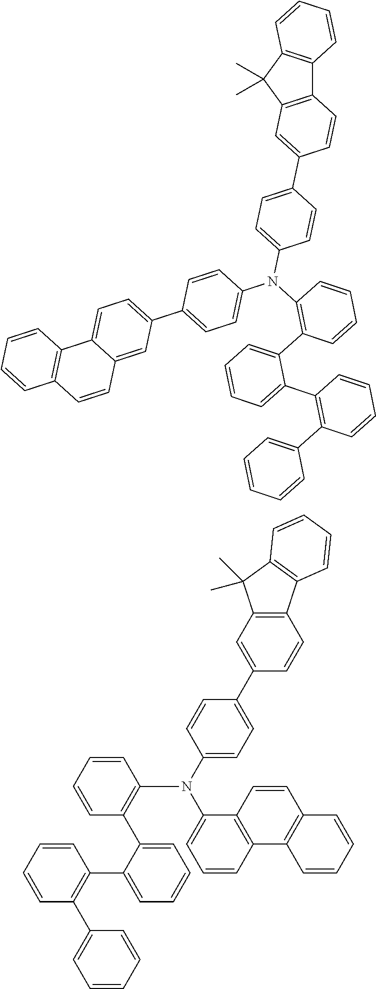

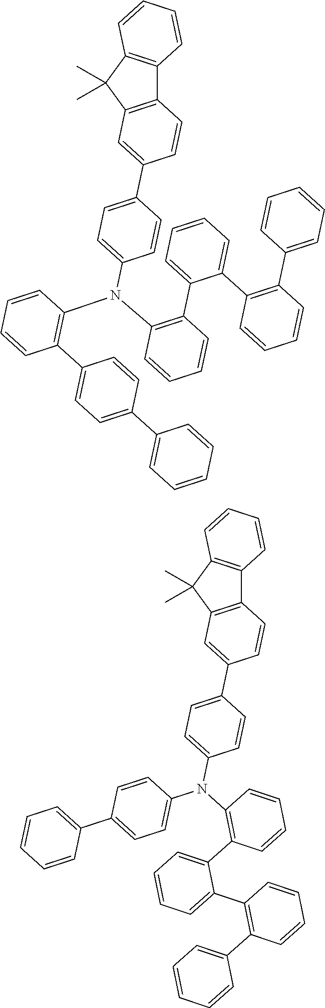

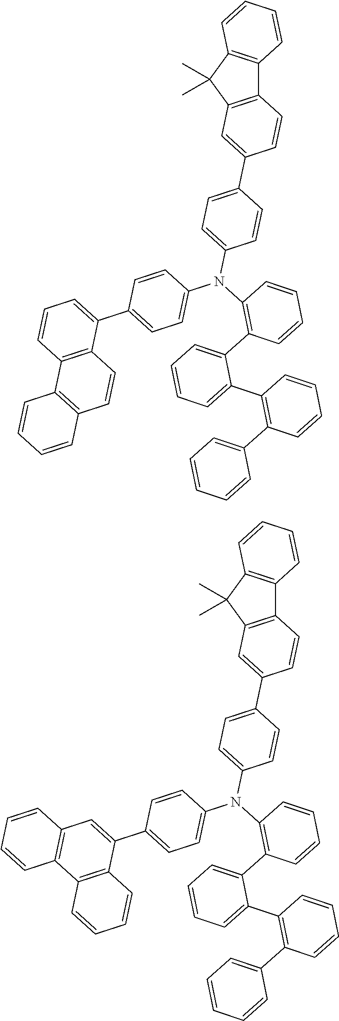

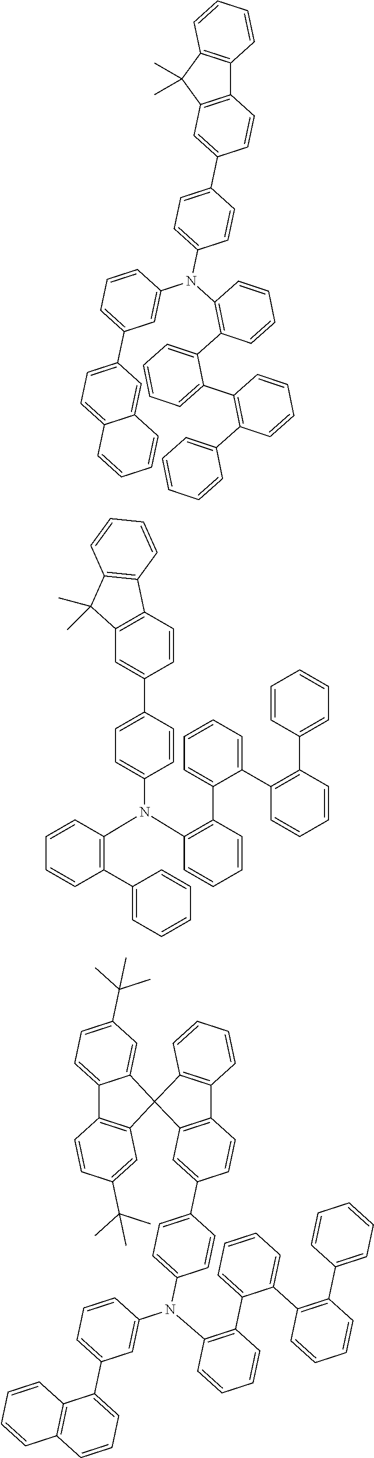

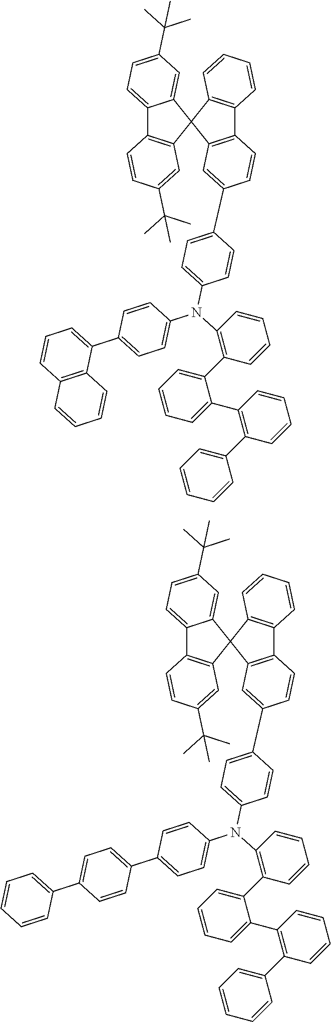

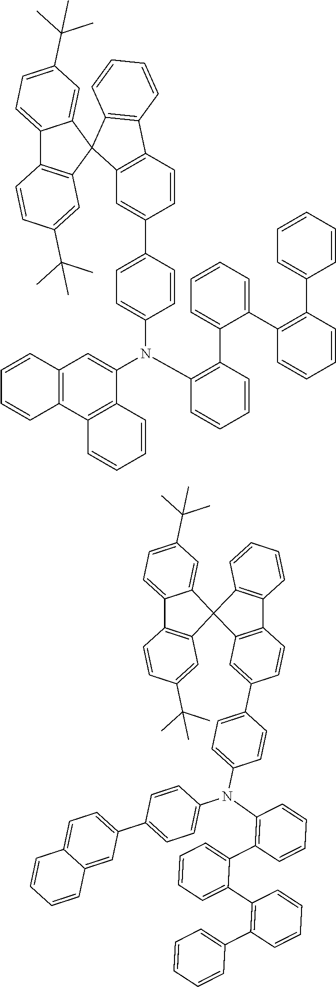

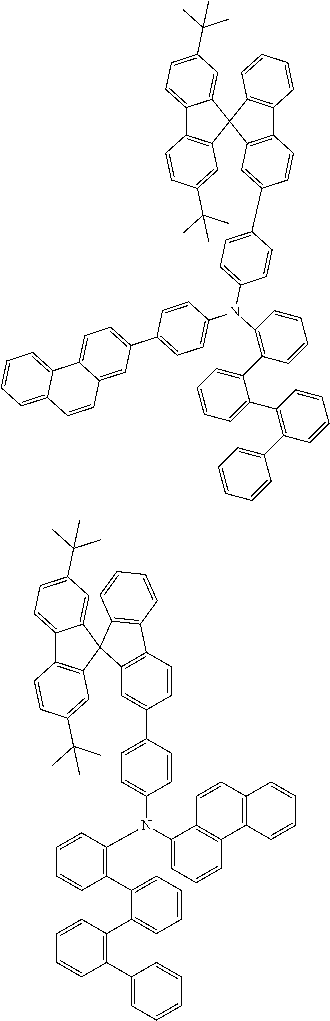 [SMILES: CC(C)(C)c1ccc2c(c1)C1(c3ccccc3-c3ccc(-c4ccc(N(c5ccc(-c6ccc7c(ccc8ccccc87)c6)cc5)c5ccccc5-c5ccccc5-c5ccccc5-c5ccccc5)cc4)cc31)c1cc(C(C)(C)C)ccc1-2 ; CC(C)(C)c1ccc2c(c1)C1(c3ccccc3-c3ccc(-c4ccc(N(c5ccccc5-c5ccccc5-c5ccccc5-c5ccccc5)c5cccc6c5ccc5ccccc56)cc4)cc31)c1cc(C(C)(C)C)ccc1-2]

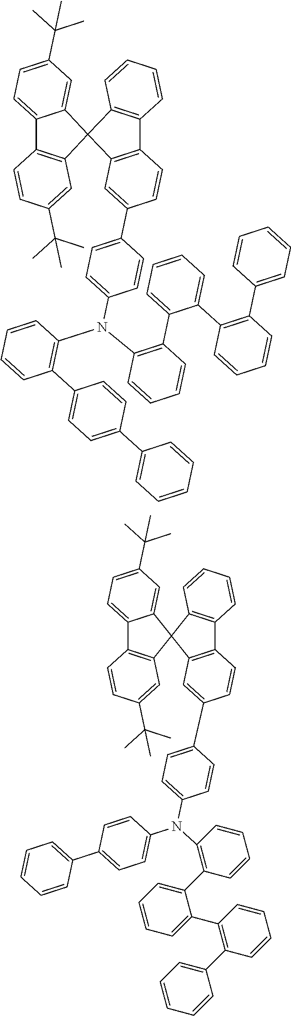

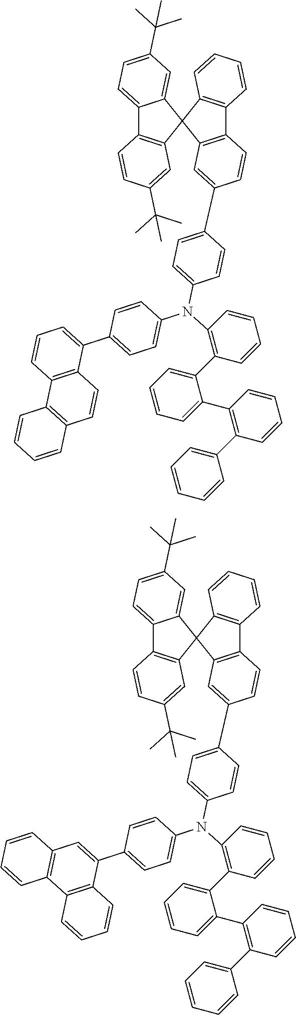

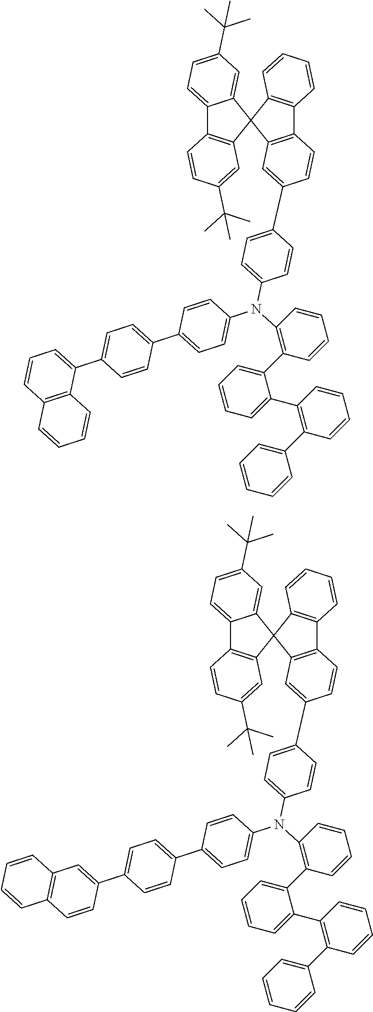

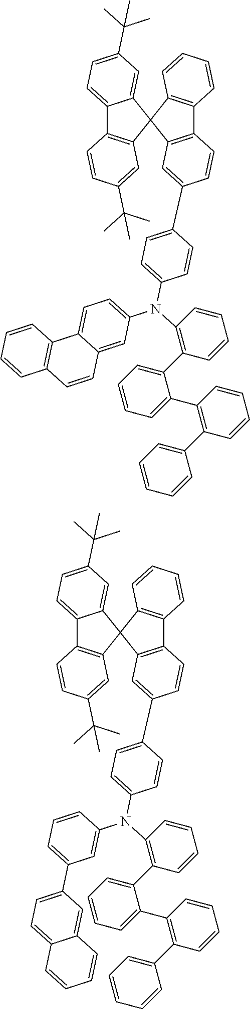 [SMILES: CC(C)(C)c1ccc2c(c1)C1(c3ccccc3-c3ccc(-c4ccc(N(c5ccc6c(ccc7ccccc76)c5)c5ccccc5-c5ccccc5-c5ccccc5-c5ccccc5)cc4)cc31)c1cc(C(C)(C)C)ccc1-2 ; CC(C)(C)c1ccc2c(c1)C1(c3ccccc3-c3ccc(-c4ccc(N(c5cccc(-c6ccc7ccccc7c6)c5)c5ccccc5-c5ccccc5-c5ccccc5-c5ccccc5)cc4)cc31)c1cc(C(C)(C)C)ccc1-2]

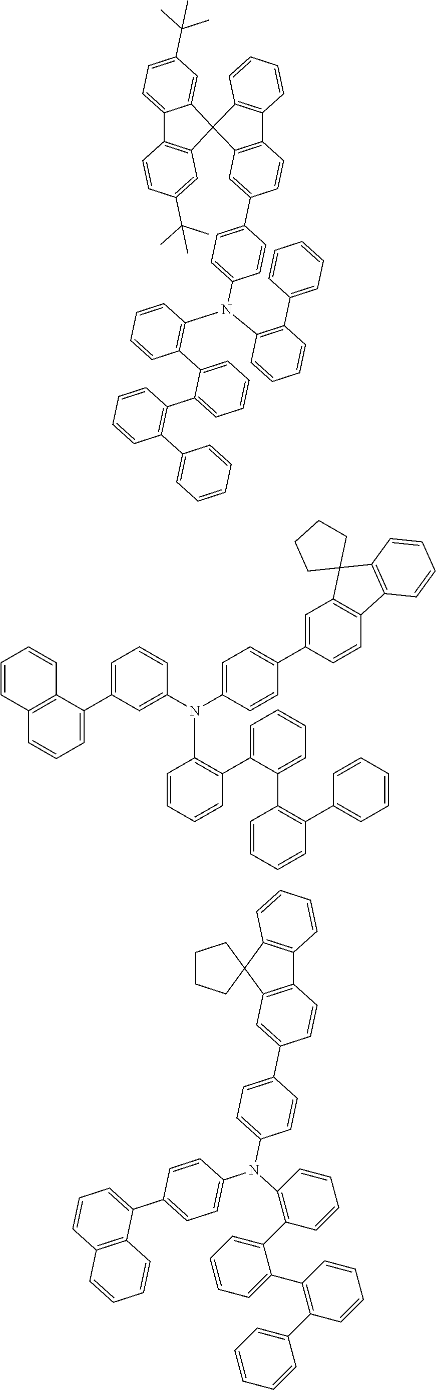

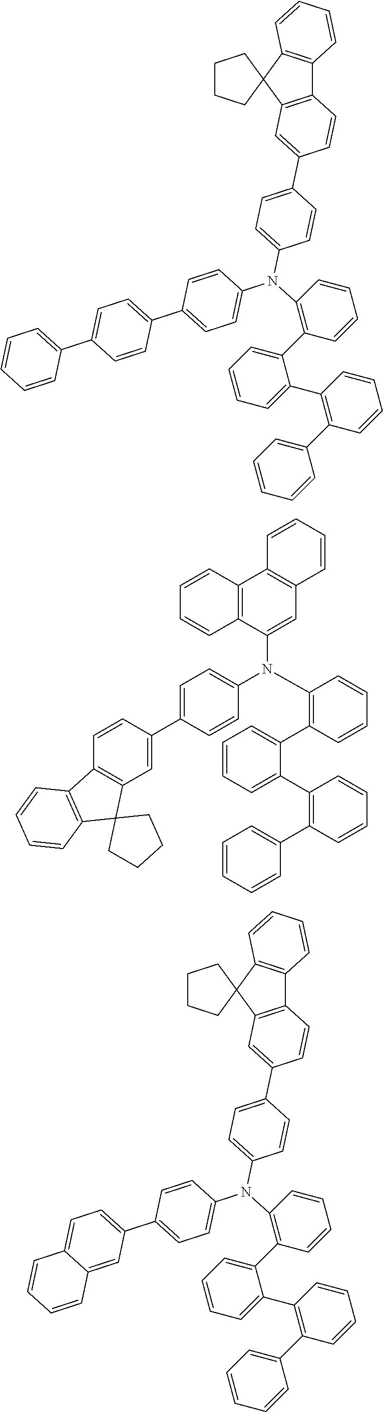

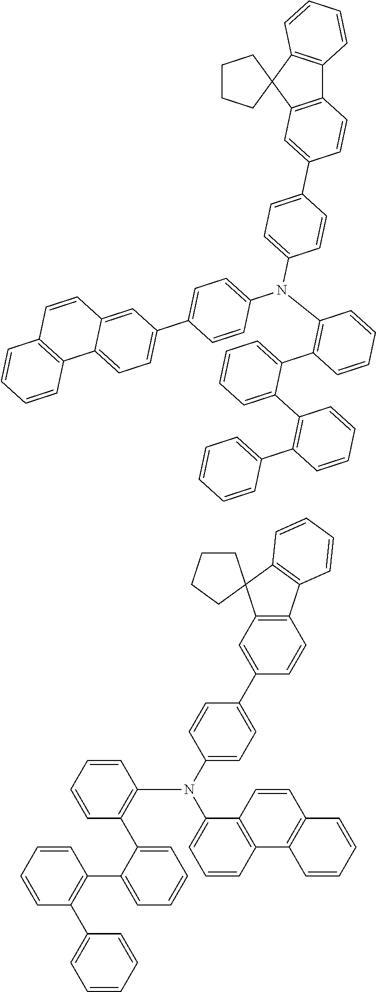 [SMILES: c1ccc(-c2ccccc2-c2ccccc2-c2ccccc2N(c2ccc(-c3ccc4c(c3)C3(CCCC3)c3ccccc3-4)cc2)c2ccc(-c3ccc4c(ccc5ccccc54)c3)cc2)cc1 ; c1ccc(-c2ccccc2-c2ccccc2-c2ccccc2N(c2ccc(-c3ccc4c(c3)C3(CCCC3)c3ccccc3-4)cc2)c2cccc3c2ccc2ccccc23)cc1]

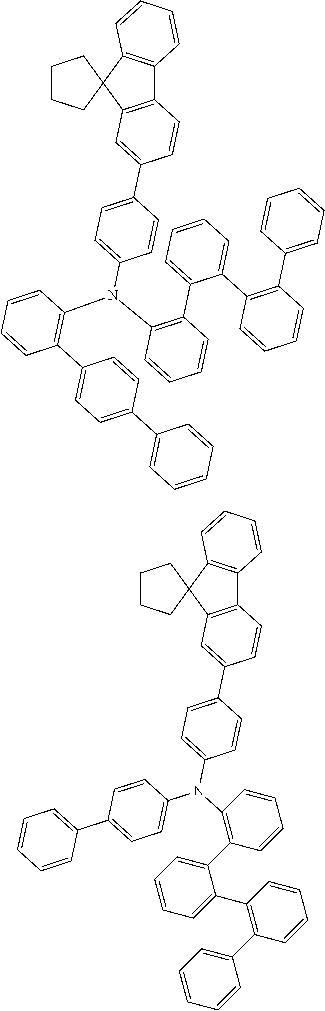

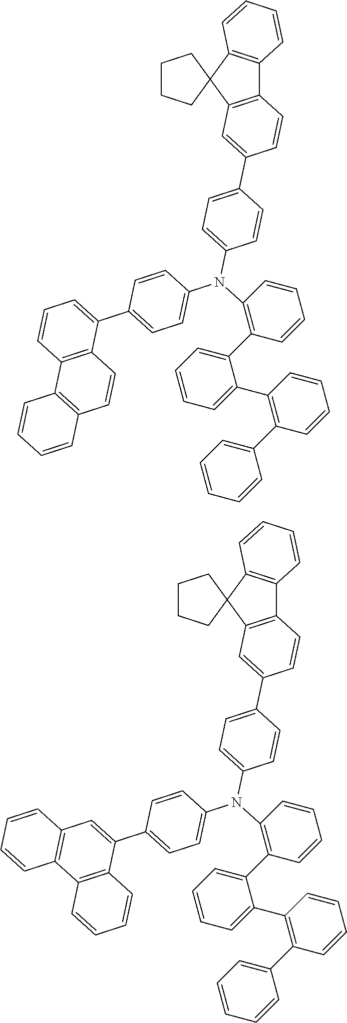

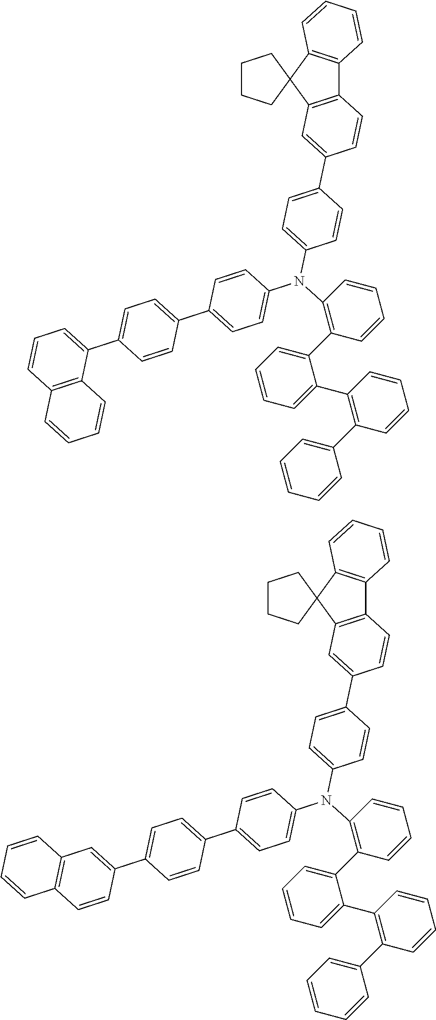

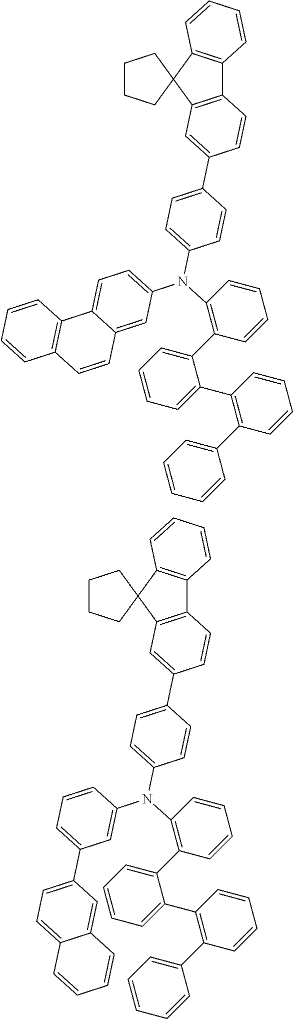

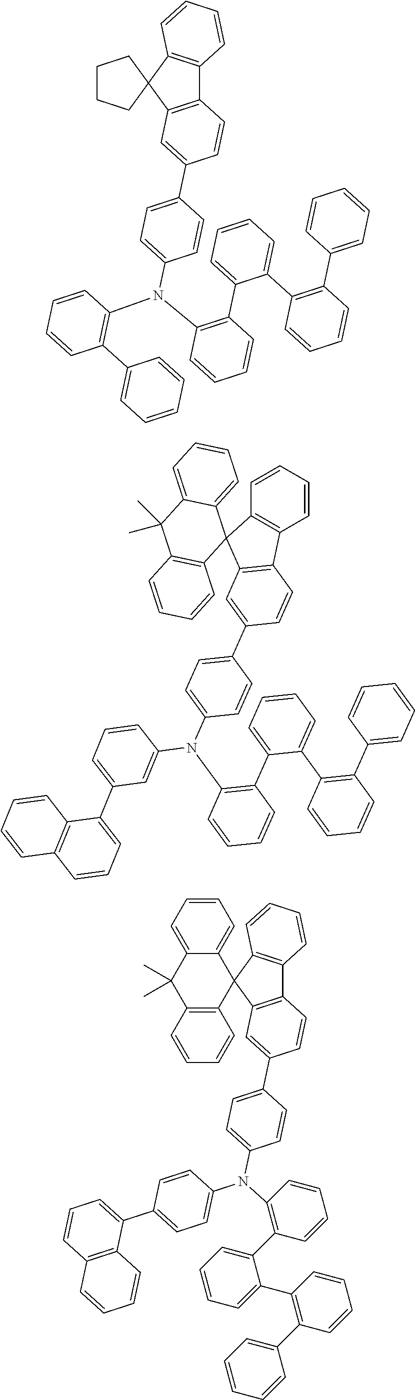

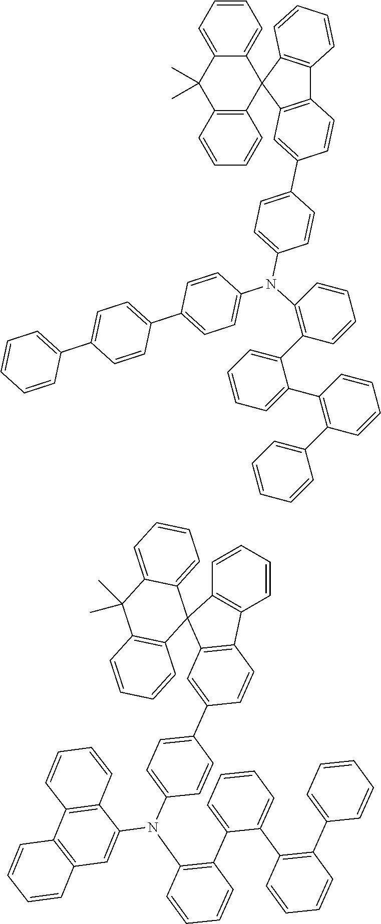 [SMILES: CC1(C)c2ccccc2C2(c3ccccc3-c3ccc(-c4ccc(N(c5ccc(-c6ccc(-c7ccccc7)cc6)cc5)c5ccccc5-c5ccccc5-c5ccccc5-c5ccccc5)cc4)cc32)c2ccccc21 ; CC1(C)c2ccccc2C2(c3ccccc3-c3ccc(-c4ccc(N(c5ccccc5-c5ccccc5-c5ccccc5-c5ccccc5)c5cc6ccccc6c6ccccc56)cc4)cc32)c2ccccc21]

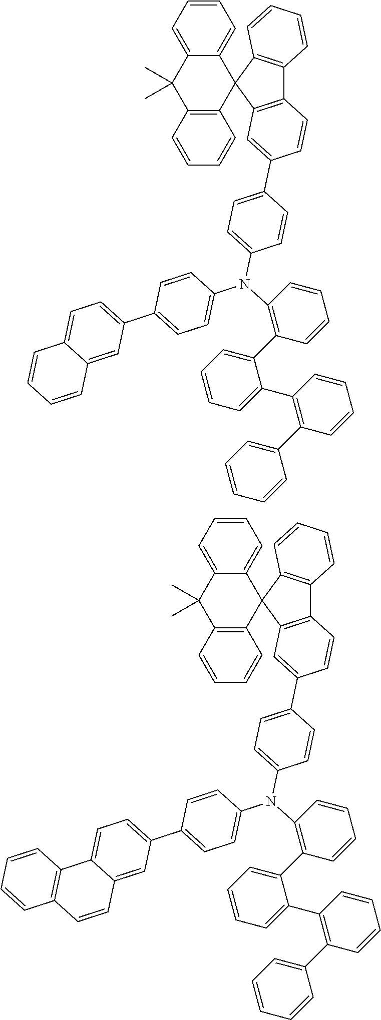

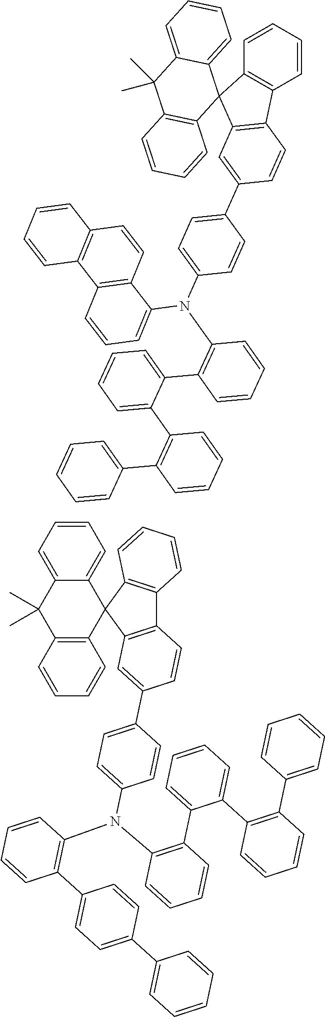

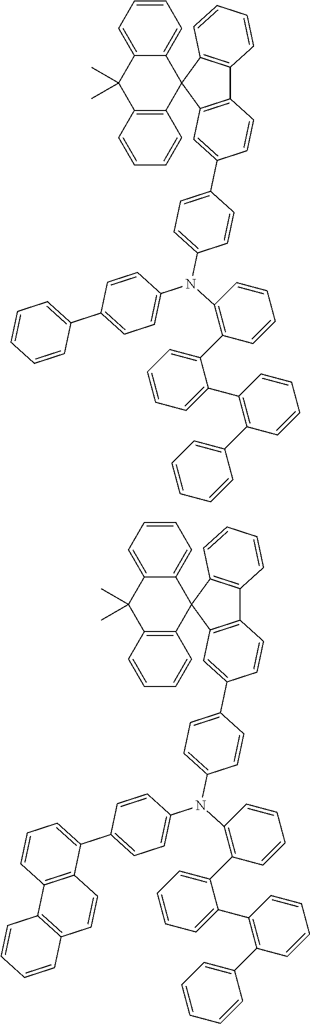

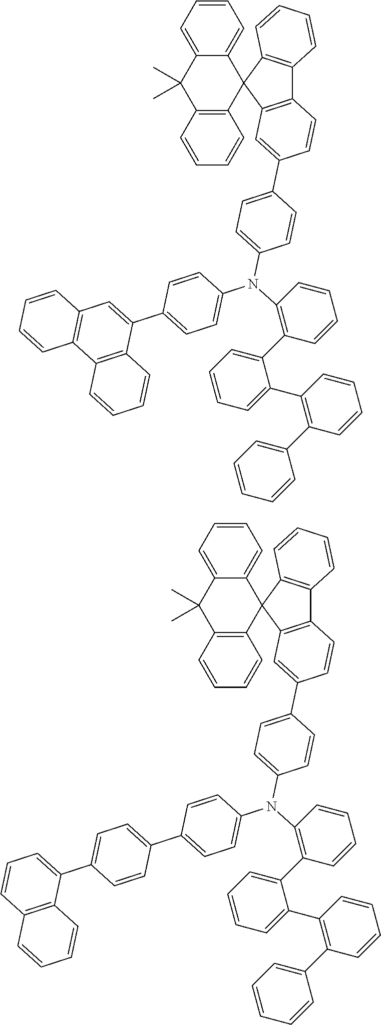

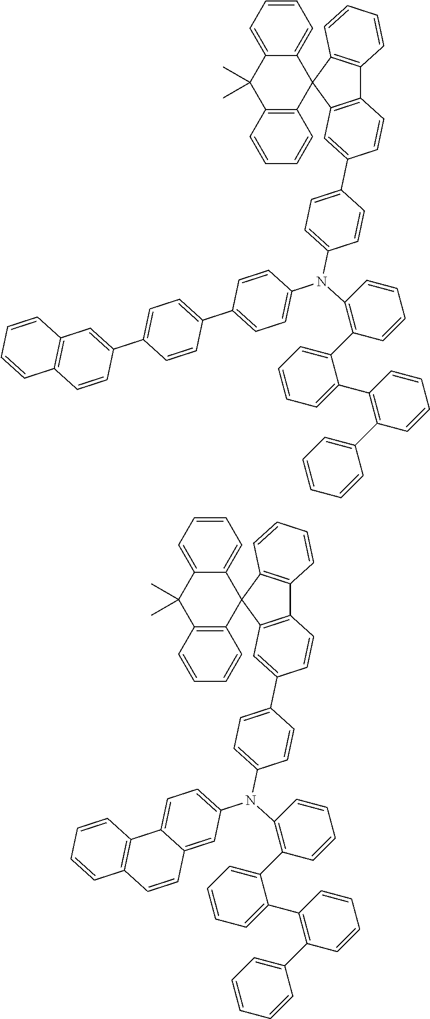

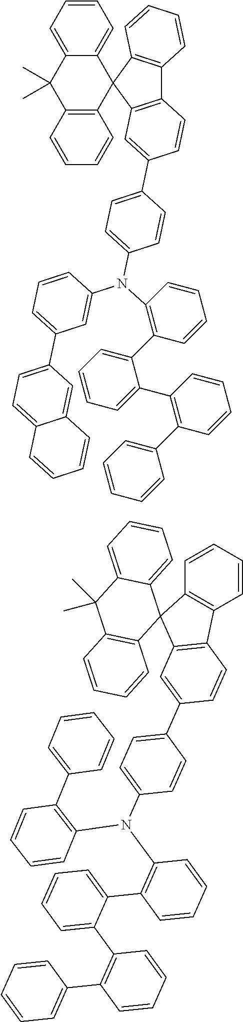

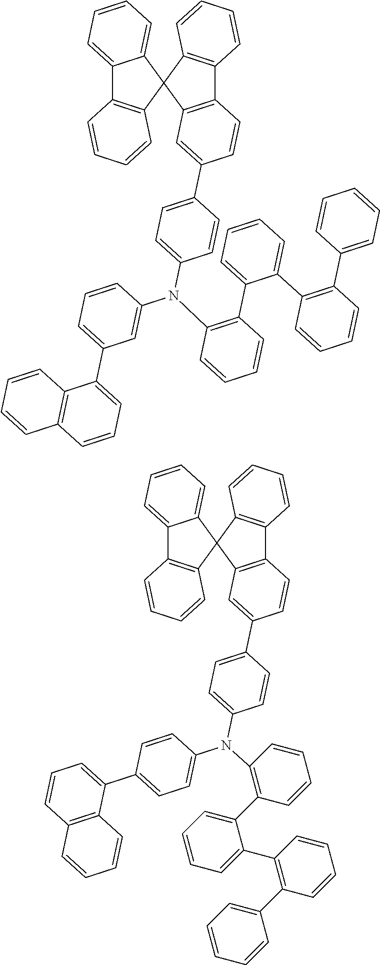 [SMILES: c1ccc(-c2ccccc2-c2ccccc2-c2ccccc2N(c2ccc(-c3ccc4c(c3)C3(c5ccccc5-c5ccccc53)c3ccccc3-4)cc2)c2cccc(-c3cccc4ccccc34)c2)cc1 ; c1ccc(-c2ccccc2-c2ccccc2-c2ccccc2N(c2ccc(-c3ccc4c(c3)C3(c5ccccc5-c5ccccc53)c3ccccc3-4)cc2)c2ccc(-c3cccc4ccccc34)cc2)cc1]

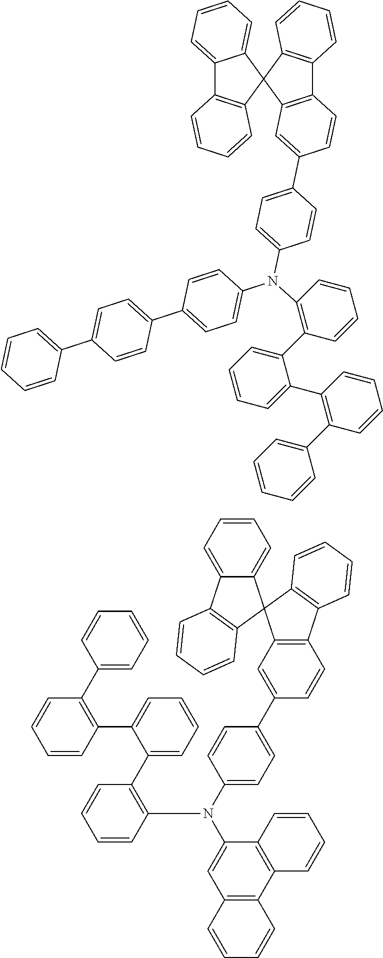

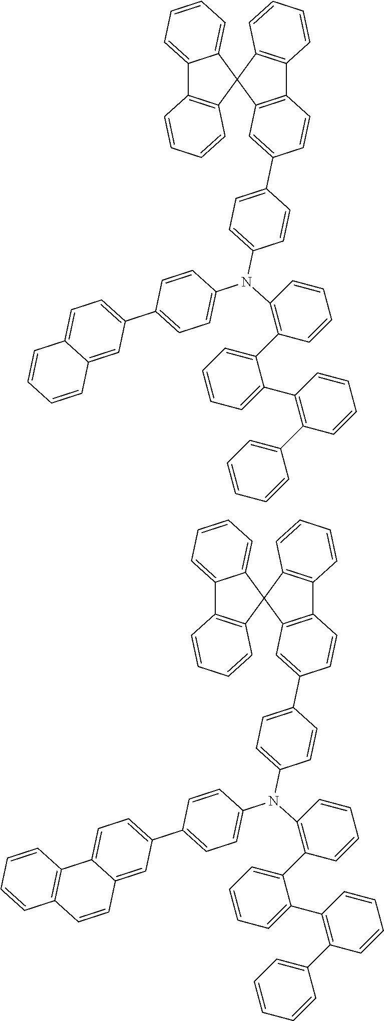

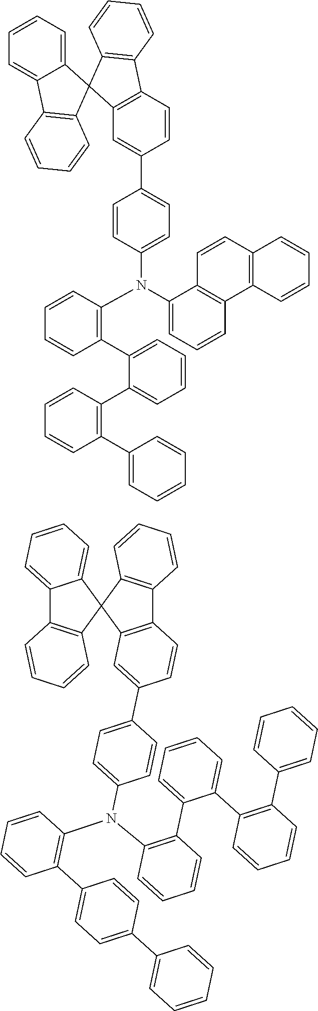

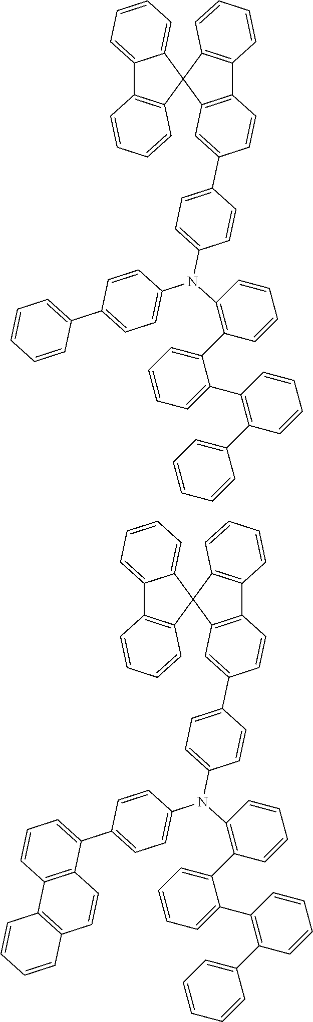

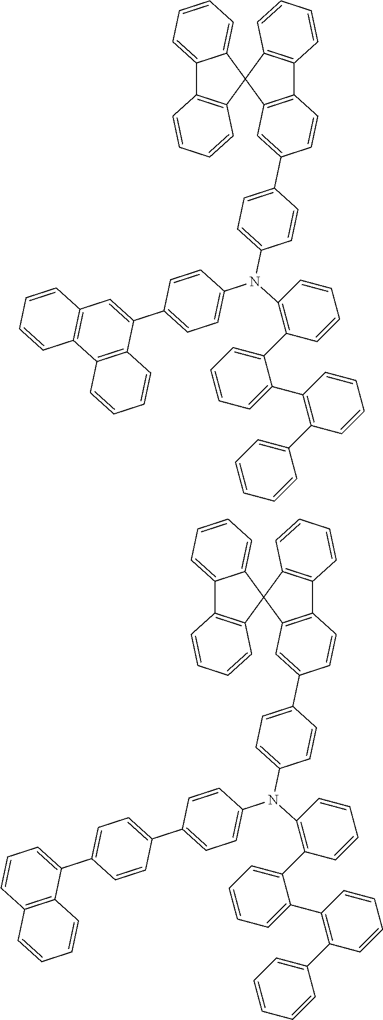

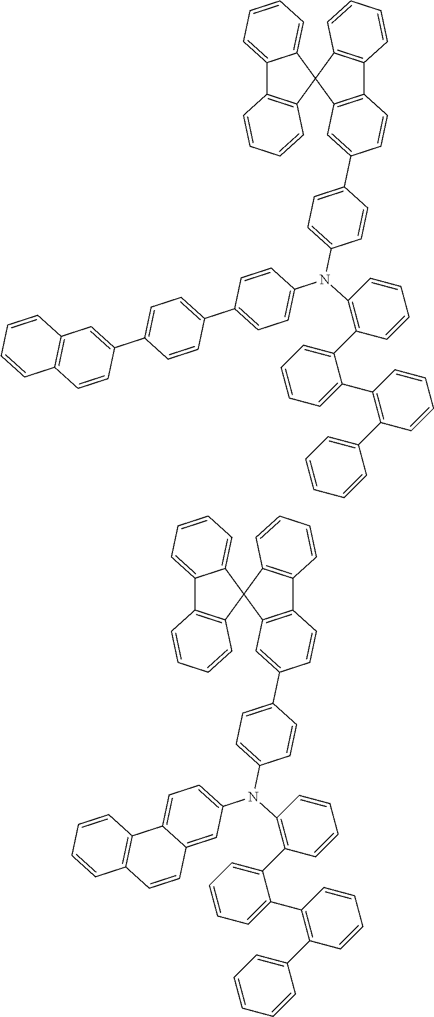

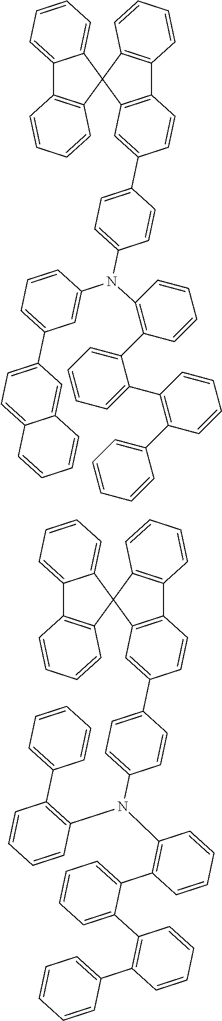

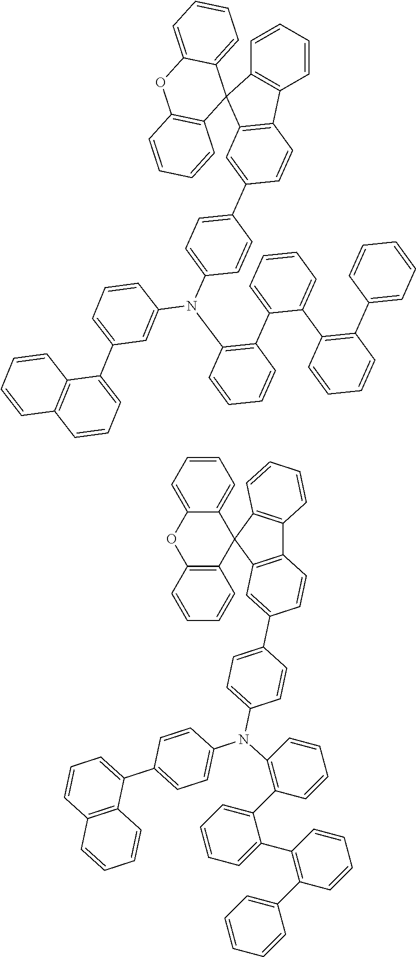 [SMILES: c1ccc(-c2ccccc2-c2ccccc2-c2ccccc2N(c2ccc(-c3ccc4c(c3)C3(c5ccccc5Oc5ccccc53)c3ccccc3-4)cc2)c2cccc(-c3cccc4ccccc34)c2)cc1 ; c1ccc(-c2ccccc2-c2ccccc2-c2ccccc2N(c2ccc(-c3ccc4c(c3)C3(c5ccccc5Oc5ccccc53)c3ccccc3-4)cc2)c2ccc(-c3cccc4ccccc34)cc2)cc1]

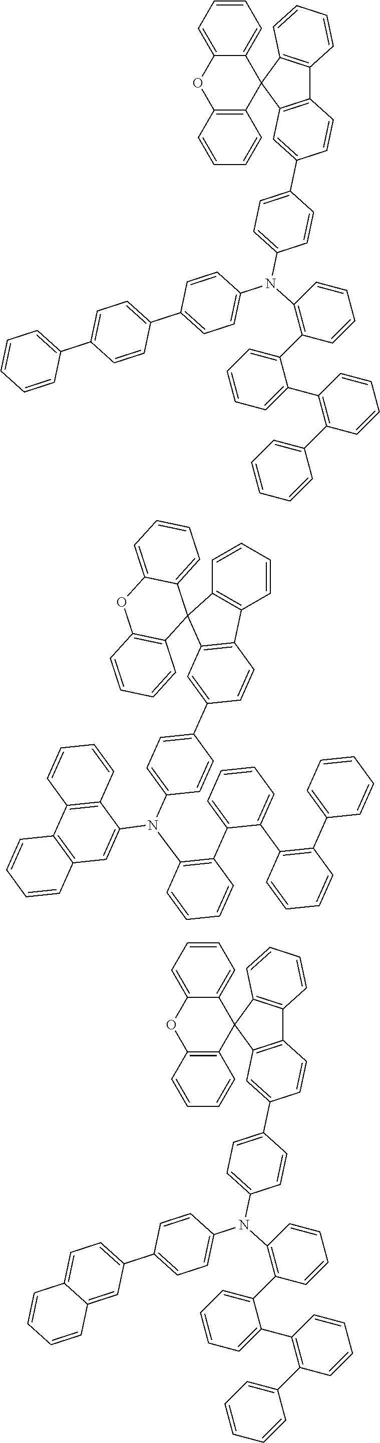

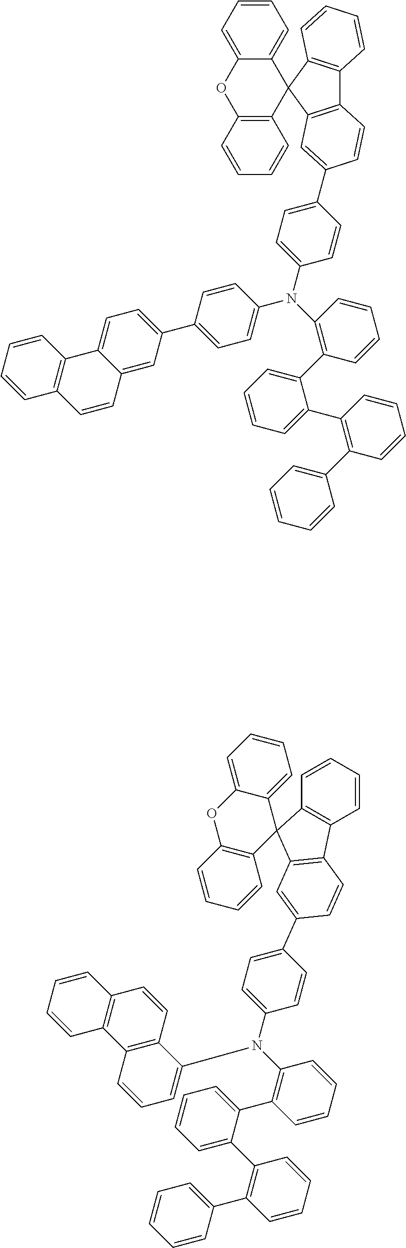 [SMILES: c1ccc(-c2ccccc2-c2ccccc2-c2ccccc2N(c2ccc(-c3ccc4c(c3)C3(c5ccccc5Oc5ccccc53)c3ccccc3-4)cc2)c2ccc(-c3ccc4c(ccc5ccccc54)c3)cc2)cc1 ; c1ccc(-c2ccccc2-c2ccccc2-c2ccccc2N(c2ccc(-c3ccc4c(c3)C3(c5ccccc5Oc5ccccc53)c3ccccc3-4)cc2)c2cccc3c2ccc2ccccc23)cc1]

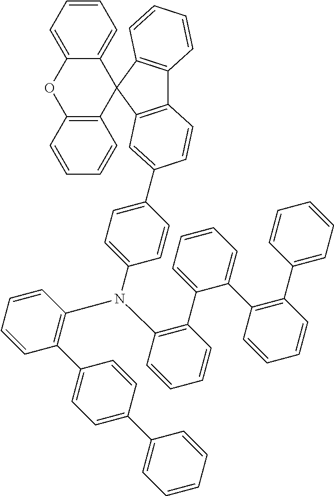 [SMILES: c1ccc(-c2ccc(-c3ccccc3N(c3ccc(-c4ccc5c(c4)C4(c6ccccc6Oc6ccccc64)c4ccccc4-5)cc3)c3ccccc3-c3ccccc3-c3ccccc3-c3ccccc3)cc2)cc1]

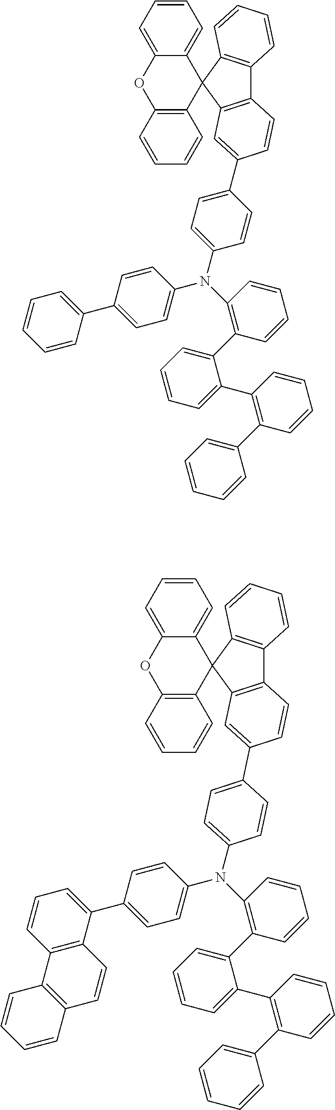 [SMILES: c1ccc(-c2ccc(N(c3ccc(-c4ccc5c(c4)C4(c6ccccc6Oc6ccccc64)c4ccccc4-5)cc3)c3ccccc3-c3ccccc3-c3ccccc3-c3ccccc3)cc2)cc1 ; c1ccc(-c2ccccc2-c2ccccc2-c2ccccc2N(c2ccc(-c3ccc4c(c3)C3(c5ccccc5Oc5ccccc53)c3ccccc3-4)cc2)c2ccc(-c3cccc4c3ccc3ccccc34)cc2)cc1]

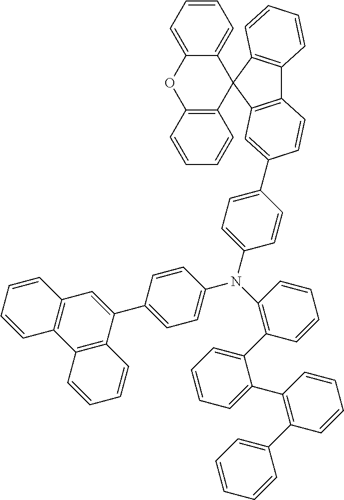 [SMILES: c1ccc(-c2ccccc2-c2ccccc2-c2ccccc2N(c2ccc(-c3ccc4c(c3)C3(c5ccccc5Oc5ccccc53)c3ccccc3-4)cc2)c2ccc(-c3cc4ccccc4c4ccccc34)cc2)cc1]

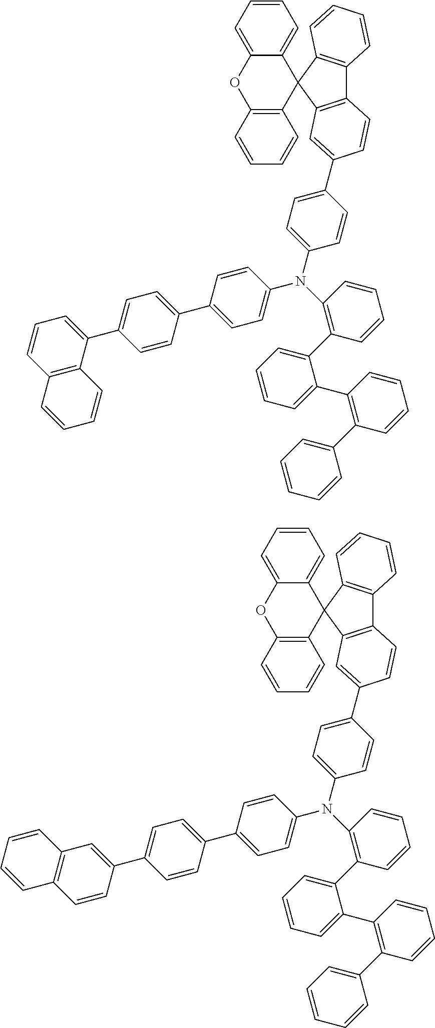

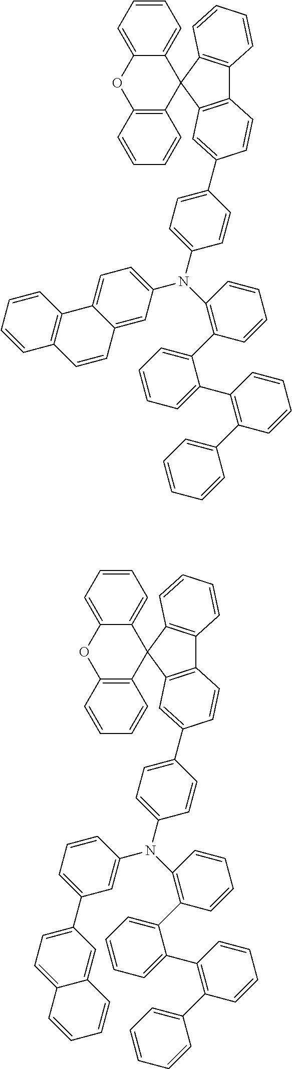 [SMILES: c1ccc(-c2ccccc2-c2ccccc2-c2ccccc2N(c2ccc(-c3ccc4c(c3)C3(c5ccccc5Oc5ccccc53)c3ccccc3-4)cc2)c2ccc3c(ccc4ccccc43)c2)cc1 ; c1ccc(-c2ccccc2-c2ccccc2-c2ccccc2N(c2ccc(-c3ccc4c(c3)C3(c5ccccc5Oc5ccccc53)c3ccccc3-4)cc2)c2cccc(-c3ccc4ccccc4c3)c2)cc1]

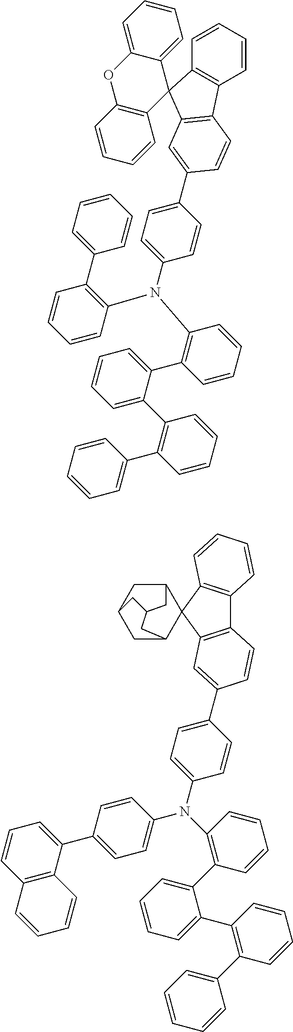 [SMILES: c1ccc(-c2ccccc2-c2ccccc2-c2ccccc2N(c2ccc(-c3ccc4c(c3)C3(c5ccccc5Oc5ccccc53)c3ccccc3-4)cc2)c2ccccc2-c2ccccc2)cc1 ; c1ccc(-c2ccccc2-c2ccccc2-c2ccccc2N(c2ccc(-c3ccc4c(c3)C3(c5ccccc5-4)C4CC5CC(C4)CC3C5)cc2)c2ccc(-c3cccc4ccccc34)cc2)cc1]

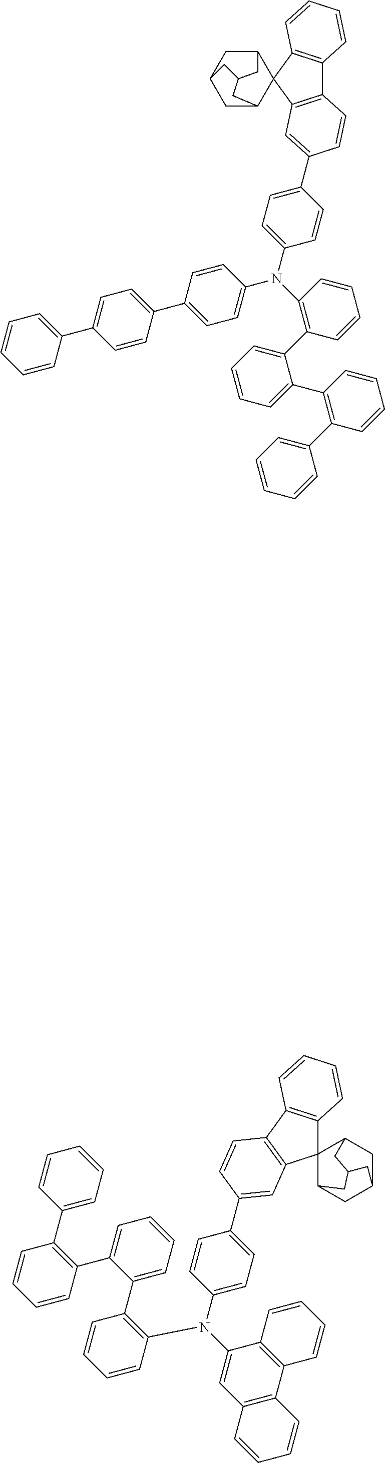 [SMILES: c1ccc(-c2ccc(-c3ccc(N(c4ccc(-c5ccc6c(c5)C5(c7ccccc7-6)C6CC7CC(C6)CC5C7)cc4)c4ccccc4-c4ccccc4-c4ccccc4-c4ccccc4)cc3)cc2)cc1 ; c1ccc(-c2ccccc2-c2ccccc2-c2ccccc2N(c2ccc(-c3ccc4c(c3)C3(c5ccccc5-4)C4CC5CC(C4)CC3C5)cc2)c2cc3ccccc3c3ccccc23)cc1]

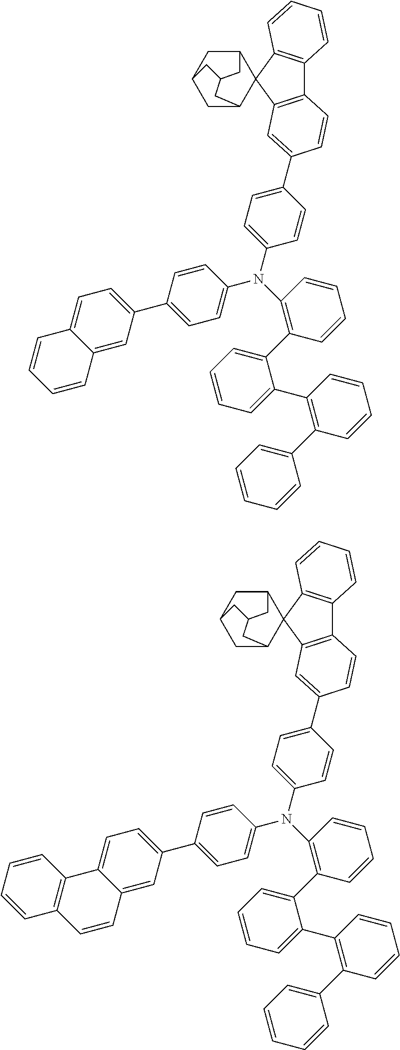 [SMILES: c1ccc(-c2ccccc2-c2ccccc2-c2ccccc2N(c2ccc(-c3ccc4c(c3)C3(c5ccccc5-4)C4CC5CC(C4)CC3C5)cc2)c2ccc(-c3ccc4ccccc4c3)cc2)cc1 ; c1ccc(-c2ccccc2-c2ccccc2-c2ccccc2N(c2ccc(-c3ccc4c(c3)C3(c5ccccc5-4)C4CC5CC(C4)CC3C5)cc2)c2ccc(-c3ccc4c(ccc5ccccc54)c3)cc2)cc1]

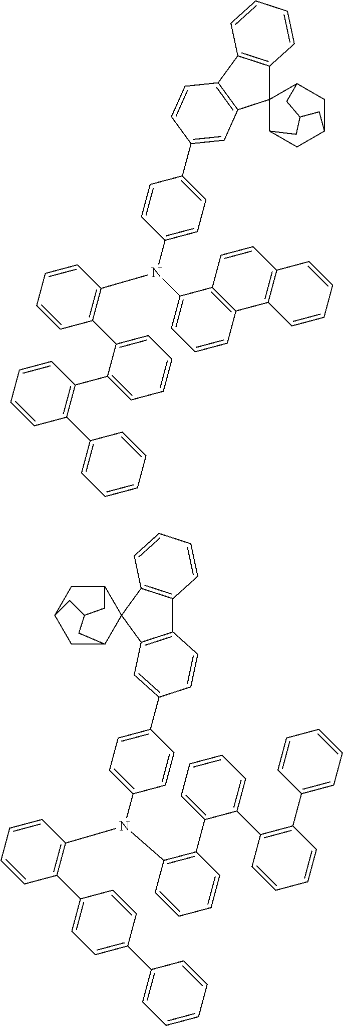 [SMILES: c1ccc(-c2ccccc2-c2ccccc2-c2ccccc2N(c2ccc(-c3ccc4c(c3)C3(c5ccccc5-4)C4CC5CC(C4)CC3C5)cc2)c2cccc3c2ccc2ccccc23)cc1 ; c1ccc(-c2ccc(-c3ccccc3N(c3ccc(-c4ccc5c(c4)C4(c6ccccc6-5)C5CC6CC(C5)CC4C6)cc3)c3ccccc3-c3ccccc3-c3ccccc3-c3ccccc3)cc2)cc1]

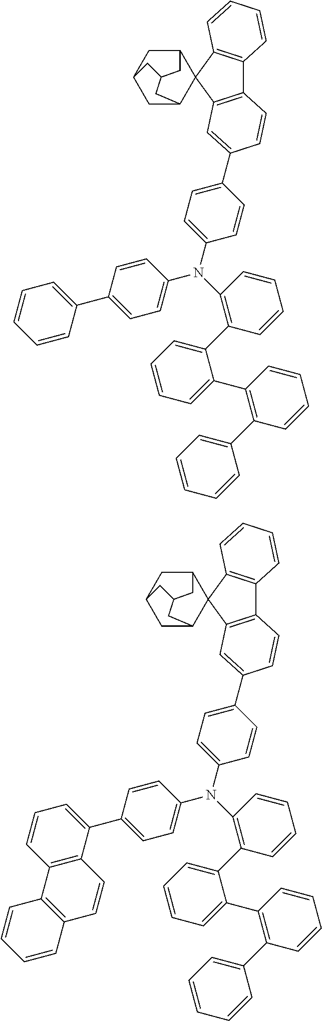

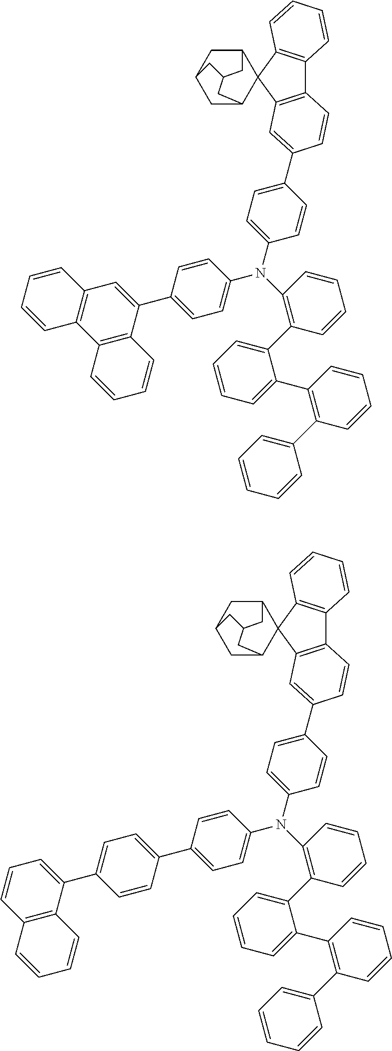 [SMILES: c1ccc(-c2ccccc2-c2ccccc2-c2ccccc2N(c2ccc(-c3ccc4c(c3)C3(c5ccccc5-4)C4CC5CC(C4)CC3C5)cc2)c2ccc(-c3cc4ccccc4c4ccccc34)cc2)cc1 ; c1ccc(-c2ccccc2-c2ccccc2-c2ccccc2N(c2ccc(-c3ccc(-c4cccc5ccccc45)cc3)cc2)c2ccc(-c3ccc4c(c3)C3(c5ccccc5-4)C4CC5CC(C4)CC3C5)cc2)cc1]

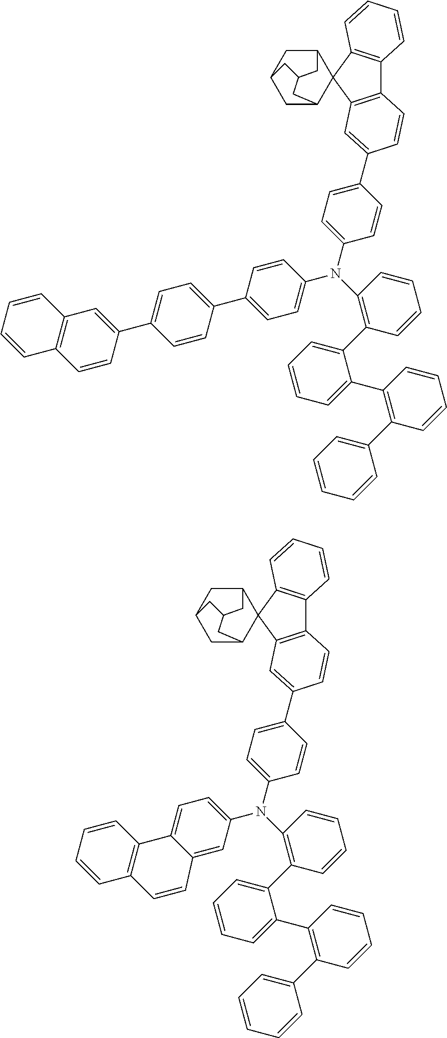 [SMILES: c1ccc(-c2ccccc2-c2ccccc2-c2ccccc2N(c2ccc(-c3ccc(-c4ccc5ccccc5c4)cc3)cc2)c2ccc(-c3ccc4c(c3)C3(c5ccccc5-4)C4CC5CC(C4)CC3C5)cc2)cc1 ; c1ccc(-c2ccccc2-c2ccccc2-c2ccccc2N(c2ccc(-c3ccc4c(c3)C3(c5ccccc5-4)C4CC5CC(C4)CC3C5)cc2)c2ccc3c(ccc4ccccc43)c2)cc1]

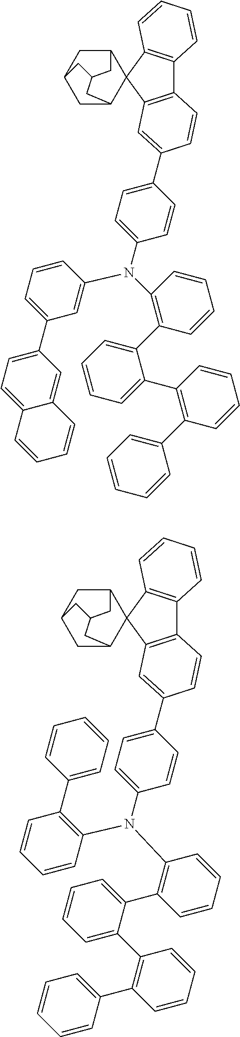

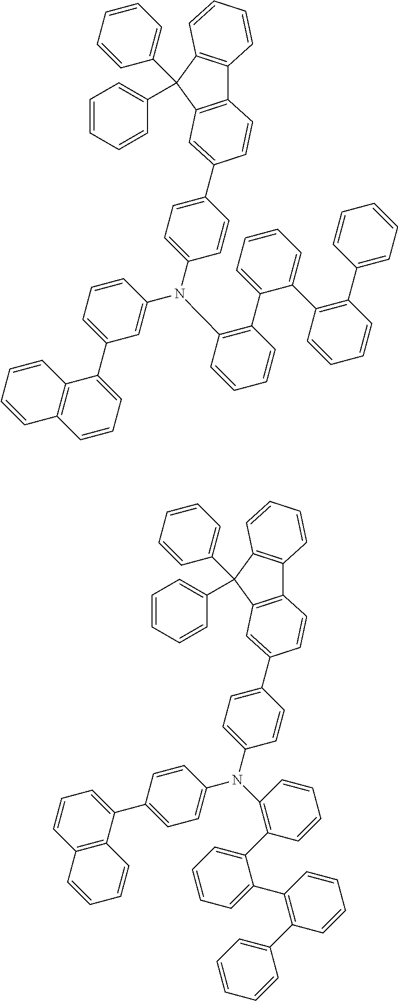 [SMILES: c1ccc(-c2ccccc2-c2ccccc2-c2ccccc2N(c2ccc(-c3ccc4c(c3)C(c3ccccc3)(c3ccccc3)c3ccccc3-4)cc2)c2cccc(-c3cccc4ccccc34)c2)cc1 ; c1ccc(-c2ccccc2-c2ccccc2-c2ccccc2N(c2ccc(-c3ccc4c(c3)C(c3ccccc3)(c3ccccc3)c3ccccc3-4)cc2)c2ccc(-c3cccc4ccccc34)cc2)cc1]

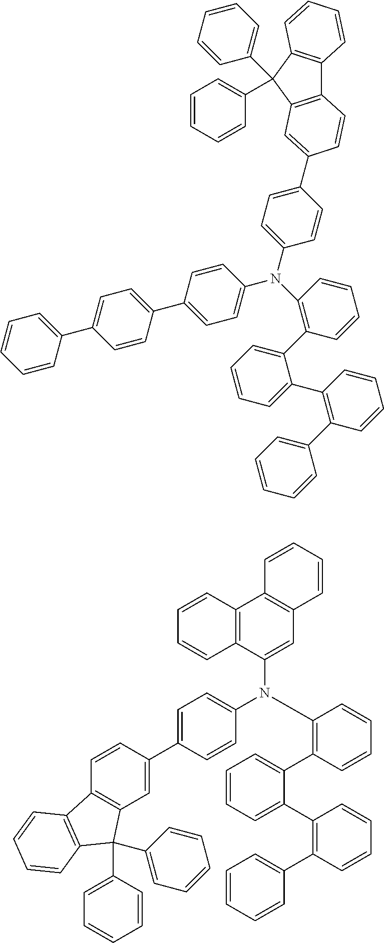 [SMILES: c1ccc(-c2ccc(-c3ccc(N(c4ccc(-c5ccc6c(c5)C(c5ccccc5)(c5ccccc5)c5ccccc5-6)cc4)c4ccccc4-c4ccccc4-c4ccccc4-c4ccccc4)cc3)cc2)cc1 ; c1ccc(-c2ccccc2-c2ccccc2-c2ccccc2N(c2ccc(-c3ccc4c(c3)C(c3ccccc3)(c3ccccc3)c3ccccc3-4)cc2)c2cc3ccccc3c3ccccc23)cc1]

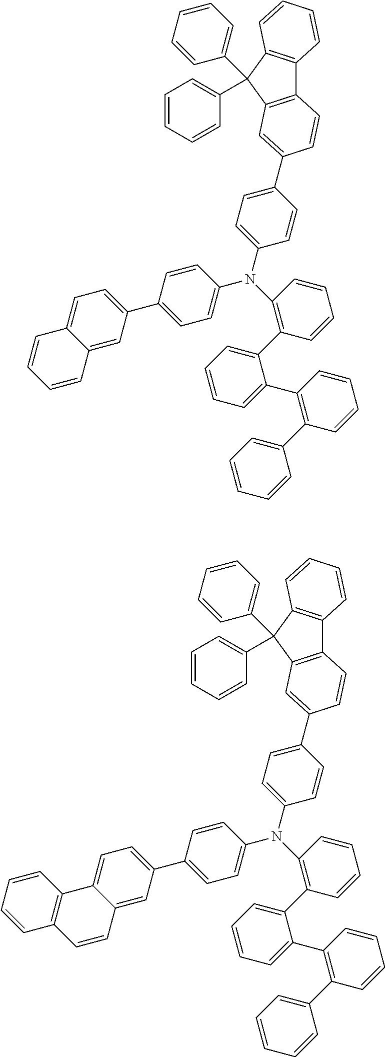 [SMILES: c1ccc(-c2ccccc2-c2ccccc2-c2ccccc2N(c2ccc(-c3ccc4c(c3)C(c3ccccc3)(c3ccccc3)c3ccccc3-4)cc2)c2ccc(-c3ccc4ccccc4c3)cc2)cc1 ; c1ccc(-c2ccccc2-c2ccccc2-c2ccccc2N(c2ccc(-c3ccc4c(c3)C(c3ccccc3)(c3ccccc3)c3ccccc3-4)cc2)c2ccc(-c3ccc4c(ccc5ccccc54)c3)cc2)cc1]

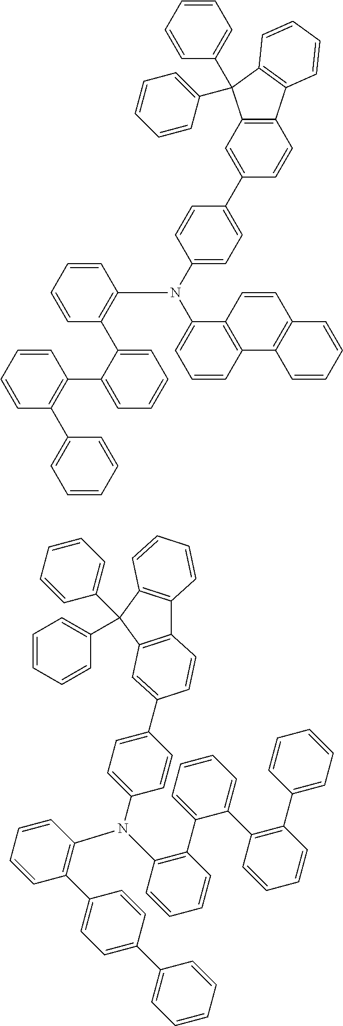

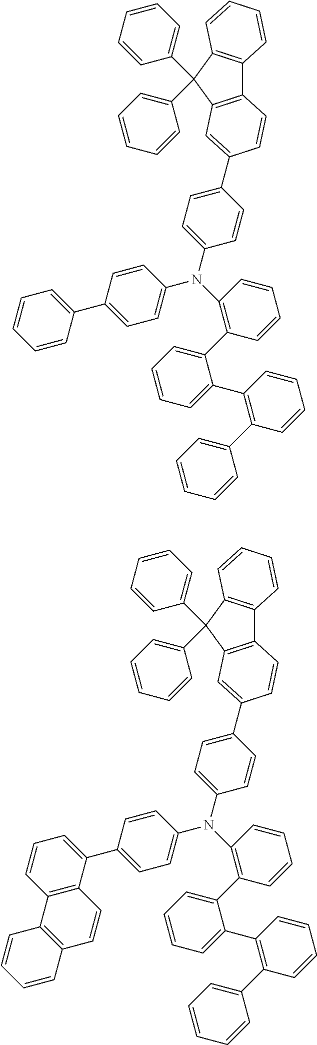 [SMILES: c1ccc(-c2ccc(N(c3ccc(-c4ccc5c(c4)C(c4ccccc4)(c4ccccc4)c4ccccc4-5)cc3)c3ccccc3-c3ccccc3-c3ccccc3-c3ccccc3)cc2)cc1 ; c1ccc(-c2ccccc2-c2ccccc2-c2ccccc2N(c2ccc(-c3ccc4c(c3)C(c3ccccc3)(c3ccccc3)c3ccccc3-4)cc2)c2ccc(-c3cccc4c3ccc3ccccc34)cc2)cc1]

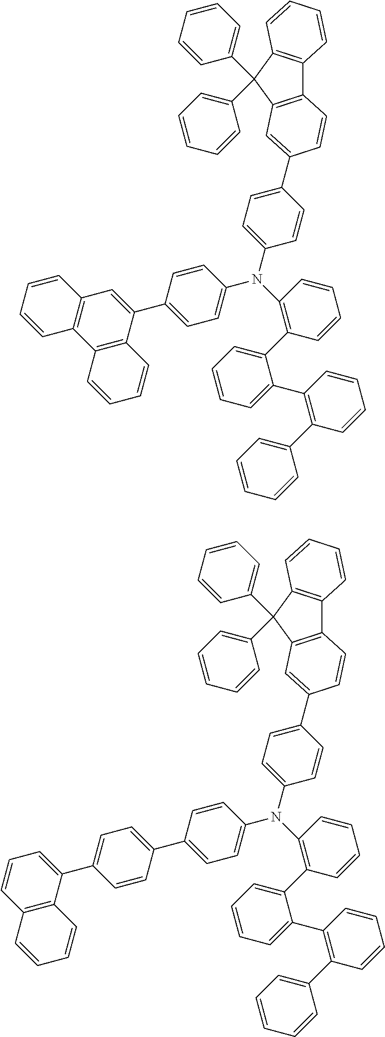 [SMILES: c1ccc(-c2ccccc2-c2ccccc2-c2ccccc2N(c2ccc(-c3ccc4c(c3)C(c3ccccc3)(c3ccccc3)c3ccccc3-4)cc2)c2ccc(-c3cc4ccccc4c4ccccc34)cc2)cc1 ; c1ccc(-c2ccccc2-c2ccccc2-c2ccccc2N(c2ccc(-c3ccc(-c4cccc5ccccc45)cc3)cc2)c2ccc(-c3ccc4c(c3)C(c3ccccc3)(c3ccccc3)c3ccccc3-4)cc2)cc1]

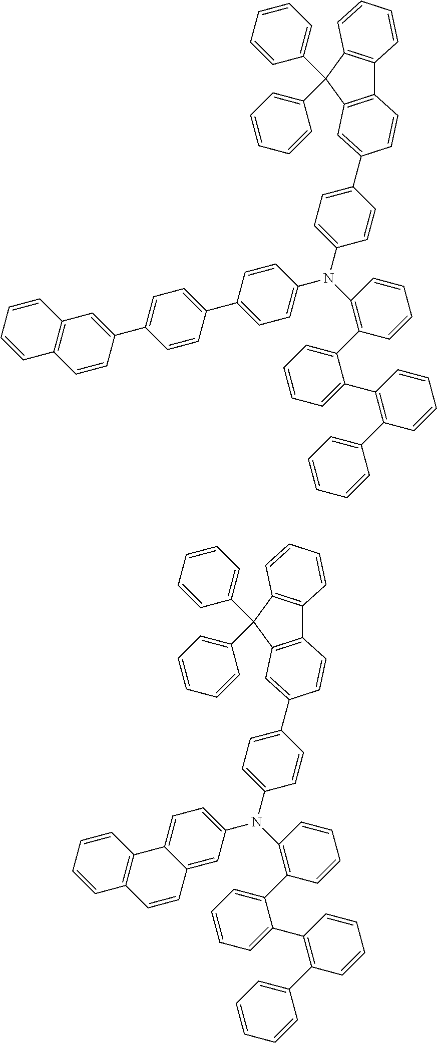 [SMILES: c1ccc(-c2ccccc2-c2ccccc2-c2ccccc2N(c2ccc(-c3ccc(-c4ccc5ccccc5c4)cc3)cc2)c2ccc(-c3ccc4c(c3)C(c3ccccc3)(c3ccccc3)c3ccccc3-4)cc2)cc1 ; c1ccc(-c2ccccc2-c2ccccc2-c2ccccc2N(c2ccc(-c3ccc4c(c3)C(c3ccccc3)(c3ccccc3)c3ccccc3-4)cc2)c2ccc3c(ccc4ccccc43)c2)cc1]

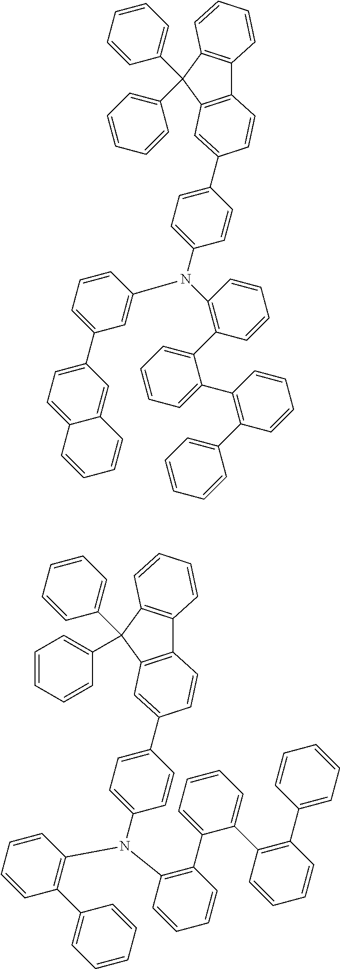 [SMILES: c1ccc(-c2ccccc2-c2ccccc2-c2ccccc2N(c2ccc(-c3ccc4c(c3)C(c3ccccc3)(c3ccccc3)c3ccccc3-4)cc2)c2cccc(-c3ccc4ccccc4c3)c2)cc1 ; c1ccc(-c2ccccc2-c2ccccc2-c2ccccc2N(c2ccc(-c3ccc4c(c3)C(c3ccccc3)(c3ccccc3)c3ccccc3-4)cc2)c2ccccc2-c2ccccc2)cc1]

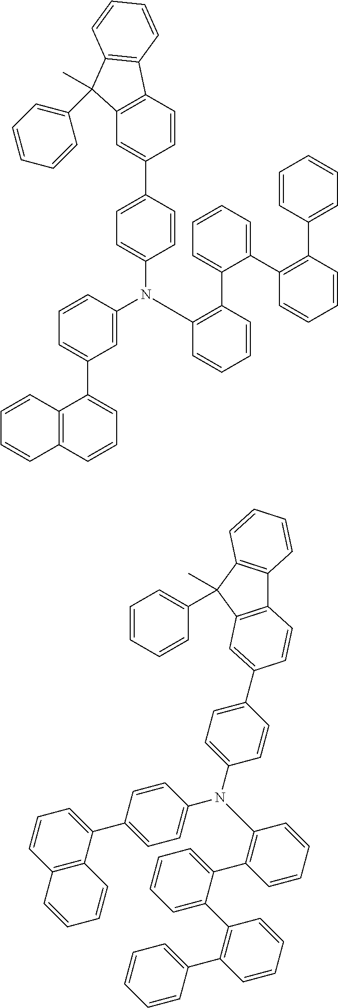 [SMILES: CC1(c2ccccc2)c2ccccc2-c2ccc(-c3ccc(N(c4cccc(-c5cccc6ccccc56)c4)c4ccccc4-c4ccccc4-c4ccccc4-c4ccccc4)cc3)cc21 ; CC1(c2ccccc2)c2ccccc2-c2ccc(-c3ccc(N(c4ccc(-c5cccc6ccccc56)cc4)c4ccccc4-c4ccccc4-c4ccccc4-c4ccccc4)cc3)cc21]

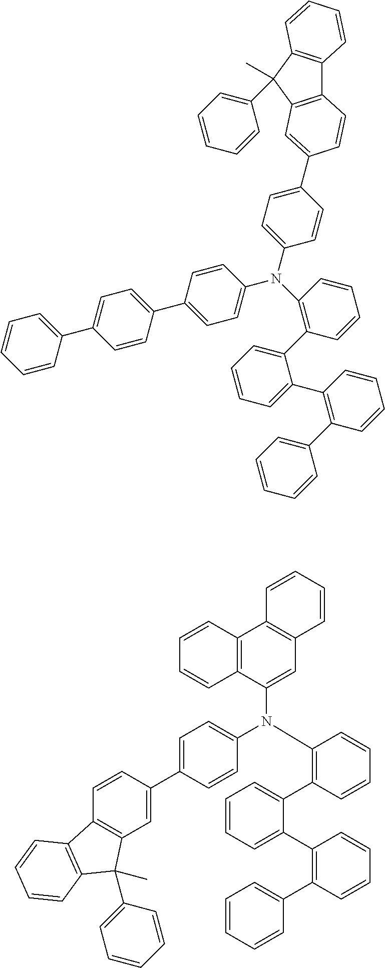 [SMILES: CC1(c2ccccc2)c2ccccc2-c2ccc(-c3ccc(N(c4ccc(-c5ccc(-c6ccccc6)cc5)cc4)c4ccccc4-c4ccccc4-c4ccccc4-c4ccccc4)cc3)cc21 ; CC1(c2ccccc2)c2ccccc2-c2ccc(-c3ccc(N(c4ccccc4-c4ccccc4-c4ccccc4-c4ccccc4)c4cc5ccccc5c5ccccc45)cc3)cc21]

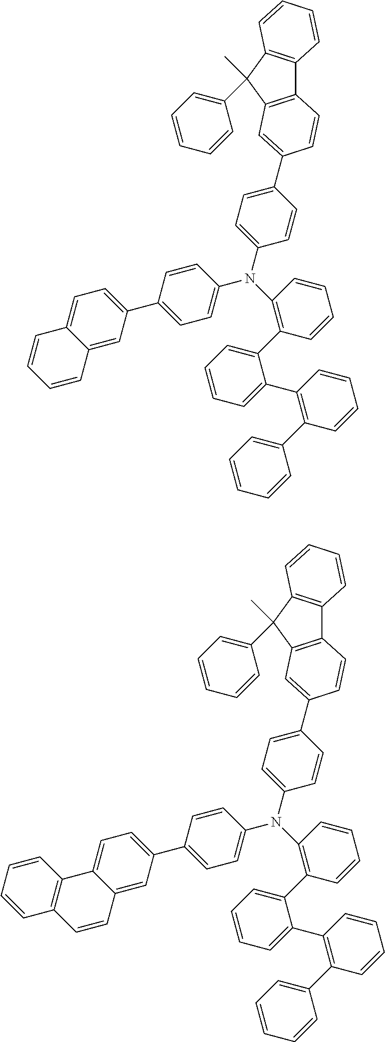 [SMILES: CC1(c2ccccc2)c2ccccc2-c2ccc(-c3ccc(N(c4ccc(-c5ccc6ccccc6c5)cc4)c4ccccc4-c4ccccc4-c4ccccc4-c4ccccc4)cc3)cc21 ; CC1(c2ccccc2)c2ccccc2-c2ccc(-c3ccc(N(c4ccc(-c5ccc6c(ccc7ccccc76)c5)cc4)c4ccccc4-c4ccccc4-c4ccccc4-c4ccccc4)cc3)cc21]

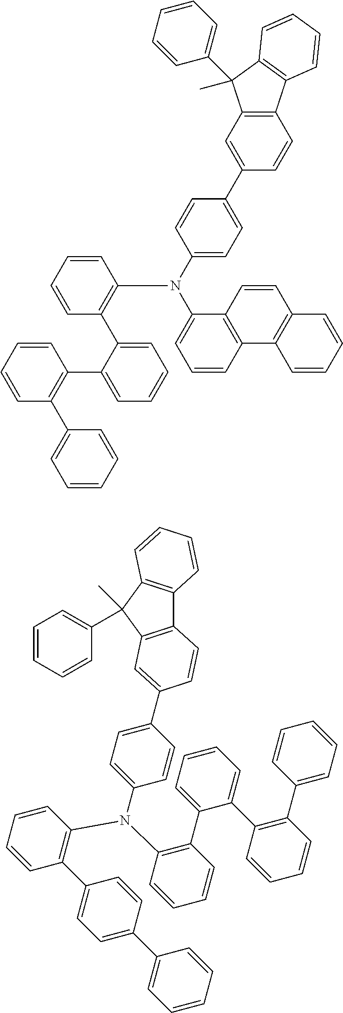 [SMILES: CC1(c2ccccc2)c2ccccc2-c2ccc(-c3ccc(N(c4ccccc4-c4ccccc4-c4ccccc4-c4ccccc4)c4cccc5c4ccc4ccccc45)cc3)cc21 ; CC1(c2ccccc2)c2ccccc2-c2ccc(-c3ccc(N(c4ccccc4-c4ccc(-c5ccccc5)cc4)c4ccccc4-c4ccccc4-c4ccccc4-c4ccccc4)cc3)cc21]

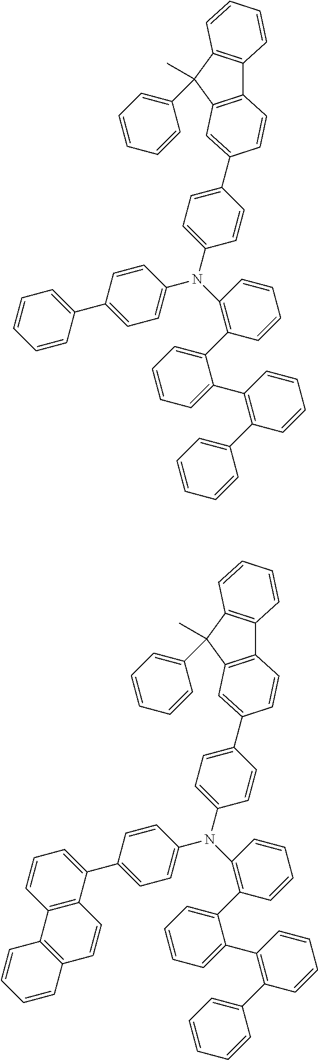 [SMILES: CC1(c2ccccc2)c2ccccc2-c2ccc(-c3ccc(N(c4ccc(-c5ccccc5)cc4)c4ccccc4-c4ccccc4-c4ccccc4-c4ccccc4)cc3)cc21 ; CC1(c2ccccc2)c2ccccc2-c2ccc(-c3ccc(N(c4ccc(-c5cccc6c5ccc5ccccc56)cc4)c4ccccc4-c4ccccc4-c4ccccc4-c4ccccc4)cc3)cc21]

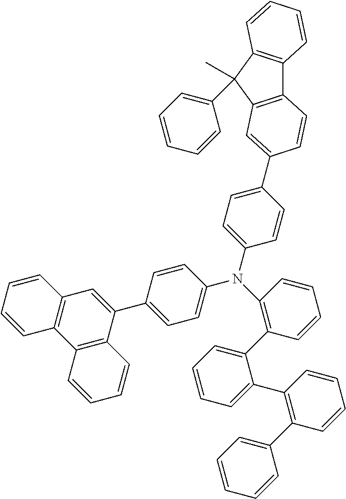 [SMILES: CC1(c2ccccc2)c2ccccc2-c2ccc(-c3ccc(N(c4ccc(-c5cc6ccccc6c6ccccc56)cc4)c4ccccc4-c4ccccc4-c4ccccc4-c4ccccc4)cc3)cc21]

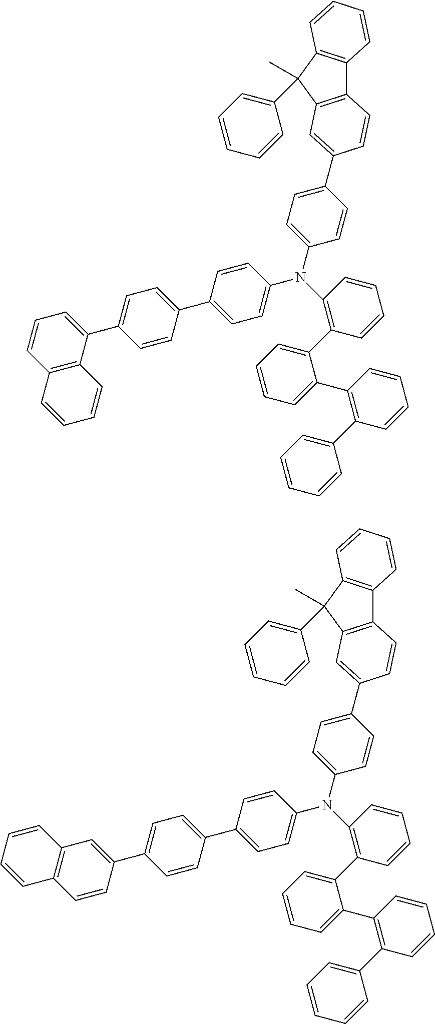 [SMILES: CC1(c2ccccc2)c2ccccc2-c2ccc(-c3ccc(N(c4ccc(-c5ccc(-c6cccc7ccccc67)cc5)cc4)c4ccccc4-c4ccccc4-c4ccccc4-c4ccccc4)cc3)cc21 ; CC1(c2ccccc2)c2ccccc2-c2ccc(-c3ccc(N(c4ccc(-c5ccc(-c6ccc7ccccc7c6)cc5)cc4)c4ccccc4-c4ccccc4-c4ccccc4-c4ccccc4)cc3)cc21]

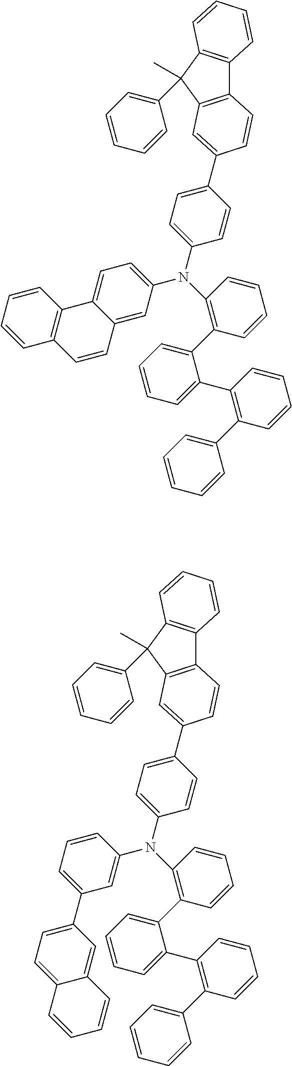 [SMILES: CC1(c2ccccc2)c2ccccc2-c2ccc(-c3ccc(N(c4ccc5c(ccc6ccccc65)c4)c4ccccc4-c4ccccc4-c4ccccc4-c4ccccc4)cc3)cc21 ; CC1(c2ccccc2)c2ccccc2-c2ccc(-c3ccc(N(c4cccc(-c5ccc6ccccc6c5)c4)c4ccccc4-c4ccccc4-c4ccccc4-c4ccccc4)cc3)cc21]

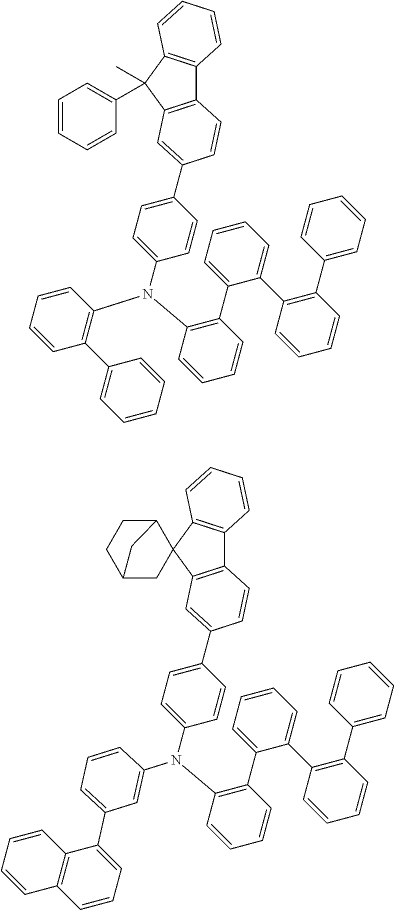 [SMILES: CC1(c2ccccc2)c2ccccc2-c2ccc(-c3ccc(N(c4ccccc4-c4ccccc4)c4ccccc4-c4ccccc4-c4ccccc4-c4ccccc4)cc3)cc21 ; c1ccc(-c2ccccc2-c2ccccc2-c2ccccc2N(c2ccc(-c3ccc4c(c3)C3(CC5CCC3C5)c3ccccc3-4)cc2)c2cccc(-c3cccc4ccccc34)c2)cc1]

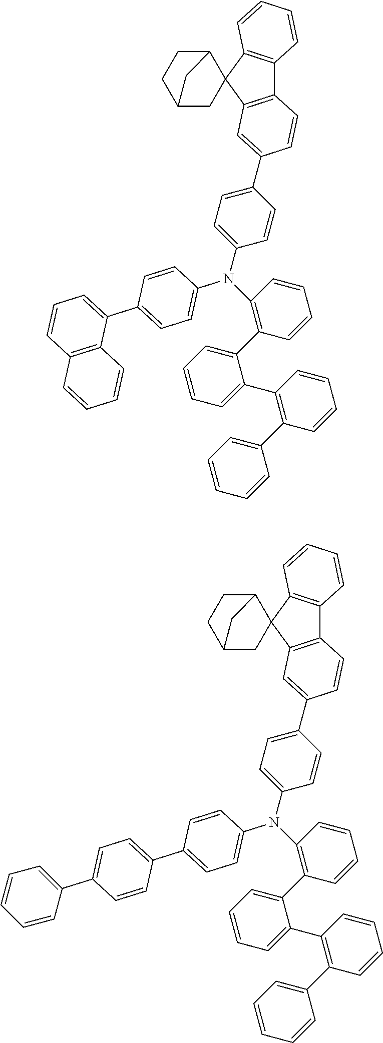 [SMILES: c1ccc(-c2ccccc2-c2ccccc2-c2ccccc2N(c2ccc(-c3ccc4c(c3)C3(CC5CCC3C5)c3ccccc3-4)cc2)c2ccc(-c3cccc4ccccc34)cc2)cc1 ; c1ccc(-c2ccc(-c3ccc(N(c4ccc(-c5ccc6c(c5)C5(CC7CCC5C7)c5ccccc5-6)cc4)c4ccccc4-c4ccccc4-c4ccccc4-c4ccccc4)cc3)cc2)cc1]

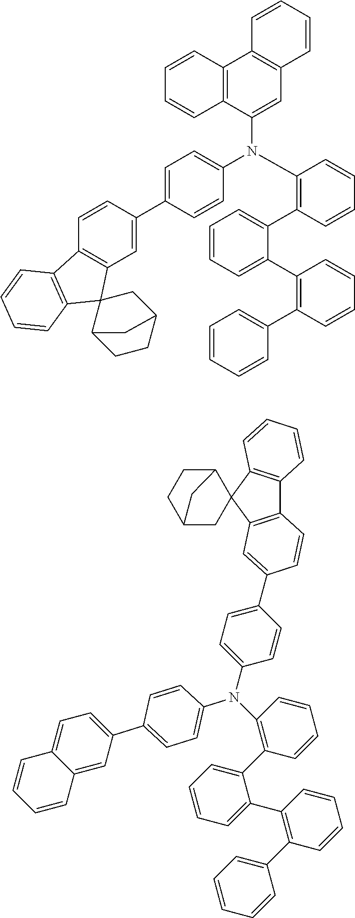 [SMILES: c1ccc(-c2ccccc2-c2ccccc2-c2ccccc2N(c2ccc(-c3ccc4c(c3)C3(CC5CCC3C5)c3ccccc3-4)cc2)c2cc3ccccc3c3ccccc23)cc1 ; c1ccc(-c2ccccc2-c2ccccc2-c2ccccc2N(c2ccc(-c3ccc4c(c3)C3(CC5CCC3C5)c3ccccc3-4)cc2)c2ccc(-c3ccc4ccccc4c3)cc2)cc1]

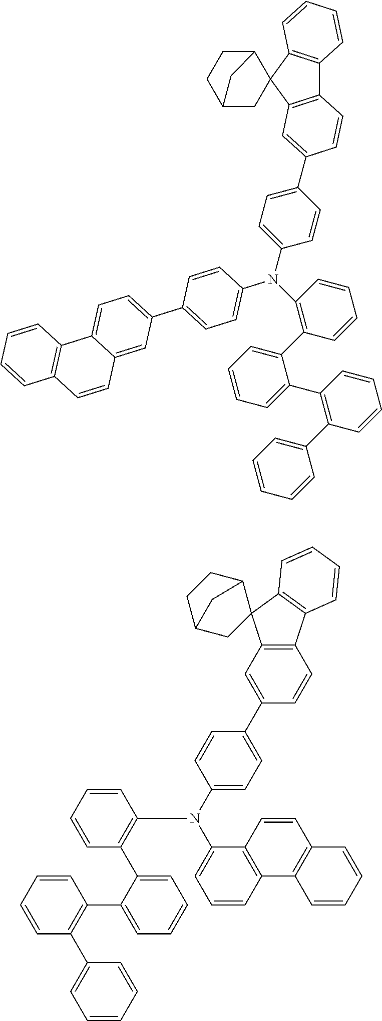 [SMILES: c1ccc(-c2ccccc2-c2ccccc2-c2ccccc2N(c2ccc(-c3ccc4c(c3)C3(CC5CCC3C5)c3ccccc3-4)cc2)c2ccc(-c3ccc4c(ccc5ccccc54)c3)cc2)cc1 ; c1ccc(-c2ccccc2-c2ccccc2-c2ccccc2N(c2ccc(-c3ccc4c(c3)C3(CC5CCC3C5)c3ccccc3-4)cc2)c2cccc3c2ccc2ccccc23)cc1]

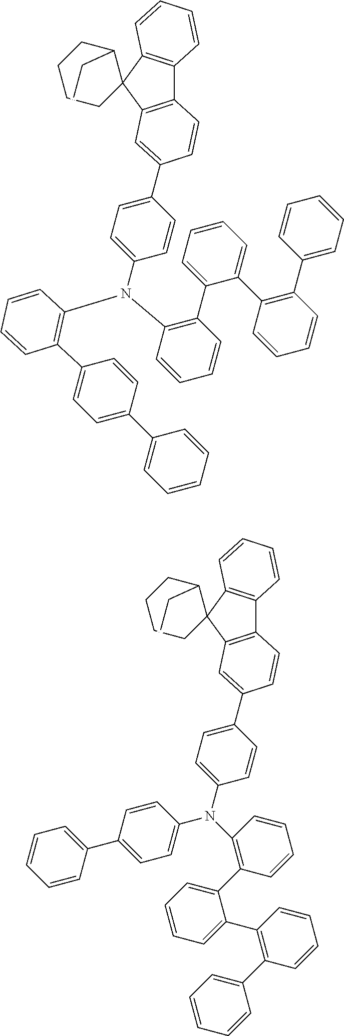

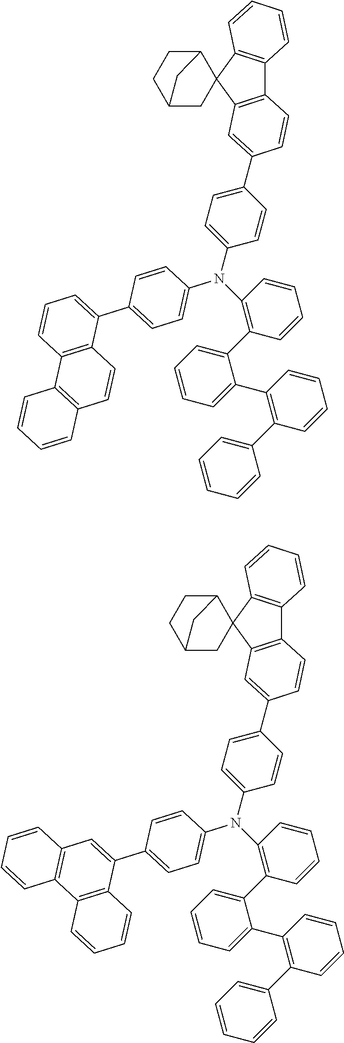 [SMILES: c1ccc(-c2ccccc2-c2ccccc2-c2ccccc2N(c2ccc(-c3ccc4c(c3)C3(CC5CCC3C5)c3ccccc3-4)cc2)c2ccc(-c3cccc4c3ccc3ccccc34)cc2)cc1 ; c1ccc(-c2ccccc2-c2ccccc2-c2ccccc2N(c2ccc(-c3ccc4c(c3)C3(CC5CCC3C5)c3ccccc3-4)cc2)c2ccc(-c3cc4ccccc4c4ccccc34)cc2)cc1]

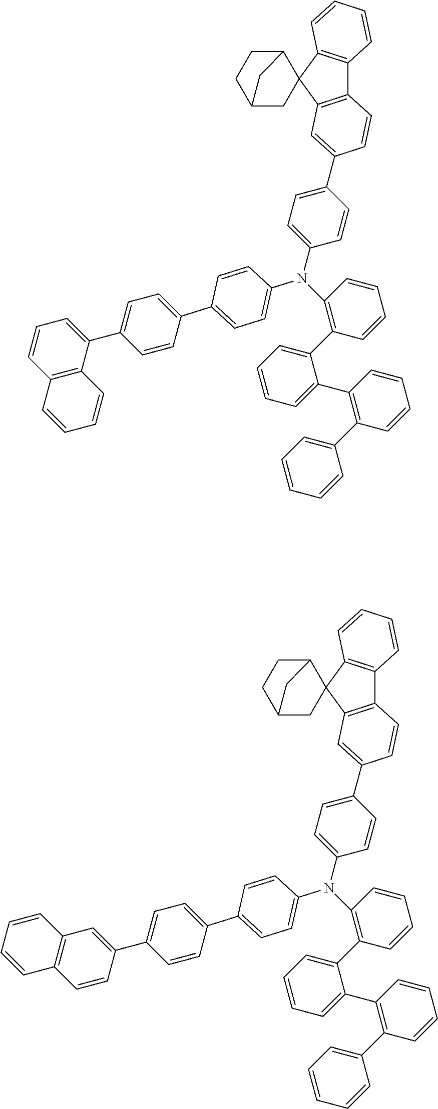 [SMILES: c1ccc(-c2ccccc2-c2ccccc2-c2ccccc2N(c2ccc(-c3ccc(-c4cccc5ccccc45)cc3)cc2)c2ccc(-c3ccc4c(c3)C3(CC5CCC3C5)c3ccccc3-4)cc2)cc1 ; c1ccc(-c2ccccc2-c2ccccc2-c2ccccc2N(c2ccc(-c3ccc(-c4ccc5ccccc5c4)cc3)cc2)c2ccc(-c3ccc4c(c3)C3(CC5CCC3C5)c3ccccc3-4)cc2)cc1]

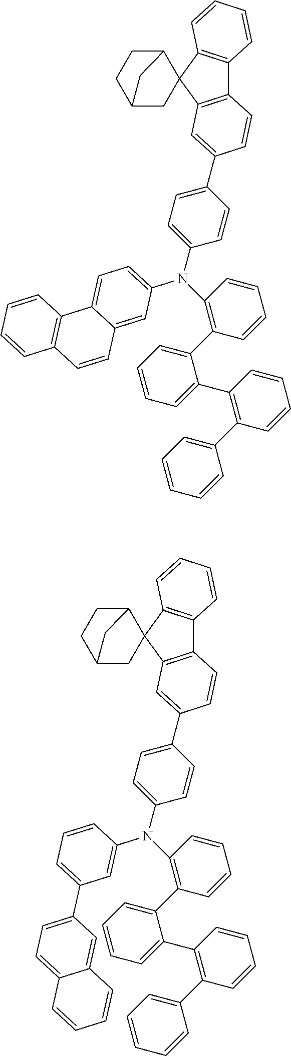 [SMILES: c1ccc(-c2ccccc2-c2ccccc2-c2ccccc2N(c2ccc(-c3ccc4c(c3)C3(CC5CCC3C5)c3ccccc3-4)cc2)c2ccc3c(ccc4ccccc43)c2)cc1 ; c1ccc(-c2ccccc2-c2ccccc2-c2ccccc2N(c2ccc(-c3ccc4c(c3)C3(CC5CCC3C5)c3ccccc3-4)cc2)c2cccc(-c3ccc4ccccc4c3)c2)cc1]

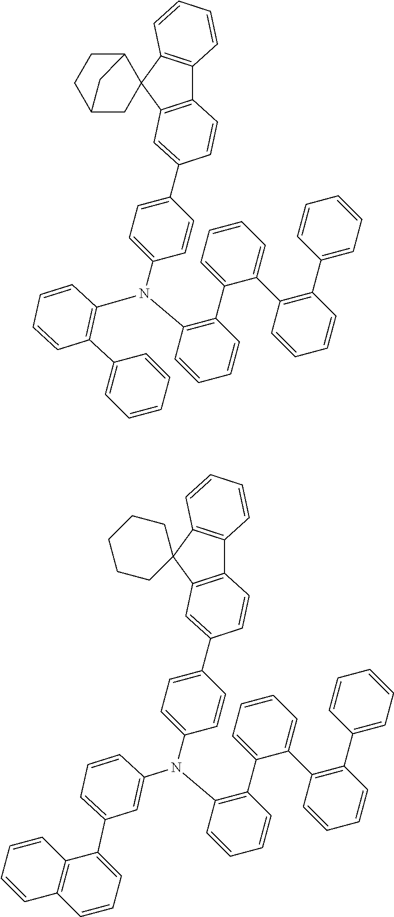 [SMILES: c1ccc(-c2ccccc2-c2ccccc2-c2ccccc2N(c2ccc(-c3ccc4c(c3)C3(CC5CCC3C5)c3ccccc3-4)cc2)c2ccccc2-c2ccccc2)cc1 ; c1ccc(-c2ccccc2-c2ccccc2-c2ccccc2N(c2ccc(-c3ccc4c(c3)C3(CCCCC3)c3ccccc3-4)cc2)c2cccc(-c3cccc4ccccc34)c2)cc1]

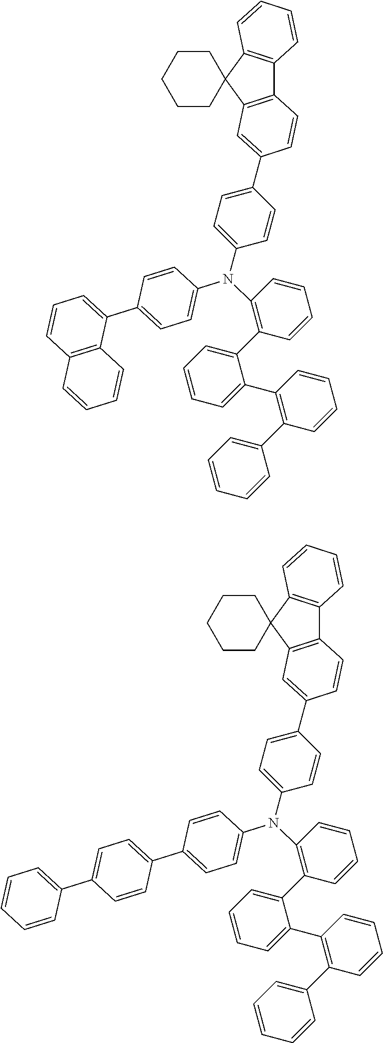 [SMILES: c1ccc(-c2ccccc2-c2ccccc2-c2ccccc2N(c2ccc(-c3ccc4c(c3)C3(CCCCC3)c3ccccc3-4)cc2)c2ccc(-c3cccc4ccccc34)cc2)cc1 ; c1ccc(-c2ccc(-c3ccc(N(c4ccc(-c5ccc6c(c5)C5(CCCCC5)c5ccccc5-6)cc4)c4ccccc4-c4ccccc4-c4ccccc4-c4ccccc4)cc3)cc2)cc1]

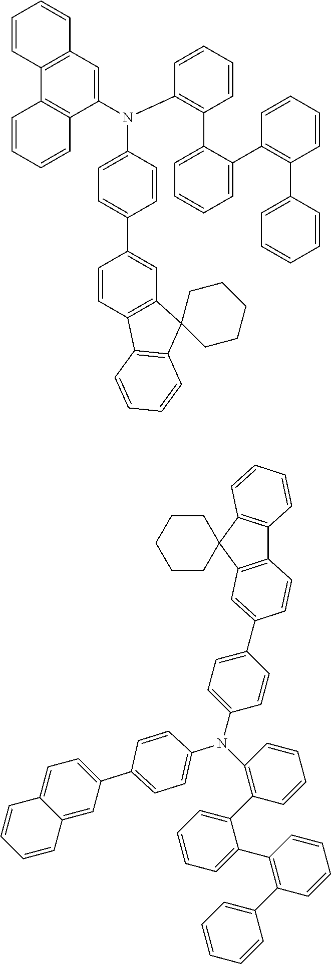 [SMILES: c1ccc(-c2ccccc2-c2ccccc2-c2ccccc2N(c2ccc(-c3ccc4c(c3)C3(CCCCC3)c3ccccc3-4)cc2)c2cc3ccccc3c3ccccc23)cc1 ; c1ccc(-c2ccccc2-c2ccccc2-c2ccccc2N(c2ccc(-c3ccc4c(c3)C3(CCCCC3)c3ccccc3-4)cc2)c2ccc(-c3ccc4ccccc4c3)cc2)cc1]

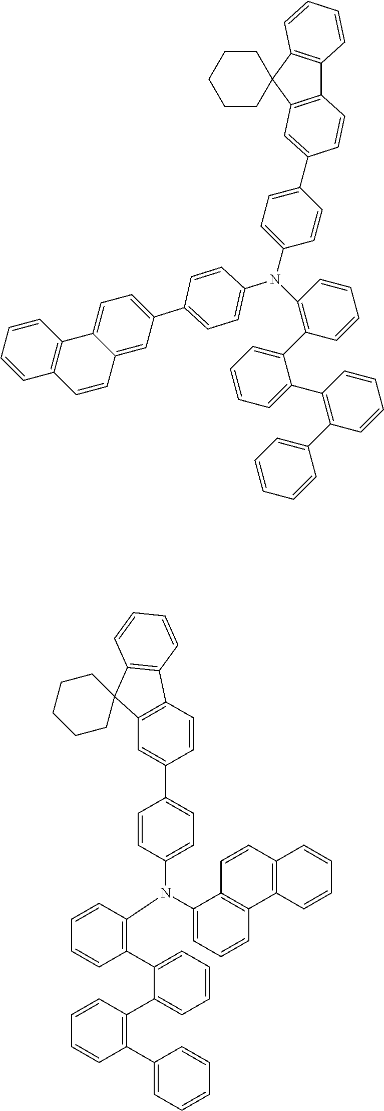 [SMILES: c1ccc(-c2ccccc2-c2ccccc2-c2ccccc2N(c2ccc(-c3ccc4c(c3)C3(CCCCC3)c3ccccc3-4)cc2)c2ccc(-c3ccc4c(ccc5ccccc54)c3)cc2)cc1 ; c1ccc(-c2ccccc2-c2ccccc2-c2ccccc2N(c2ccc(-c3ccc4c(c3)C3(CCCCC3)c3ccccc3-4)cc2)c2cccc3c2ccc2ccccc23)cc1]

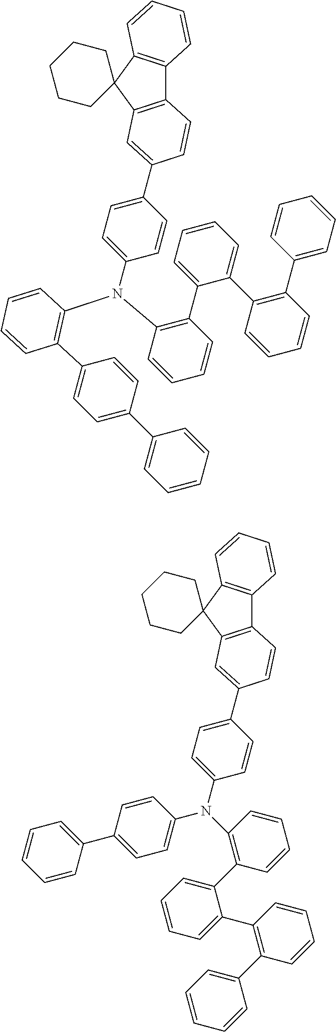 [SMILES: c1ccc(-c2ccc(-c3ccccc3N(c3ccc(-c4ccc5c(c4)C4(CCCCC4)c4ccccc4-5)cc3)c3ccccc3-c3ccccc3-c3ccccc3-c3ccccc3)cc2)cc1 ; c1ccc(-c2ccc(N(c3ccc(-c4ccc5c(c4)C4(CCCCC4)c4ccccc4-5)cc3)c3ccccc3-c3ccccc3-c3ccccc3-c3ccccc3)cc2)cc1]

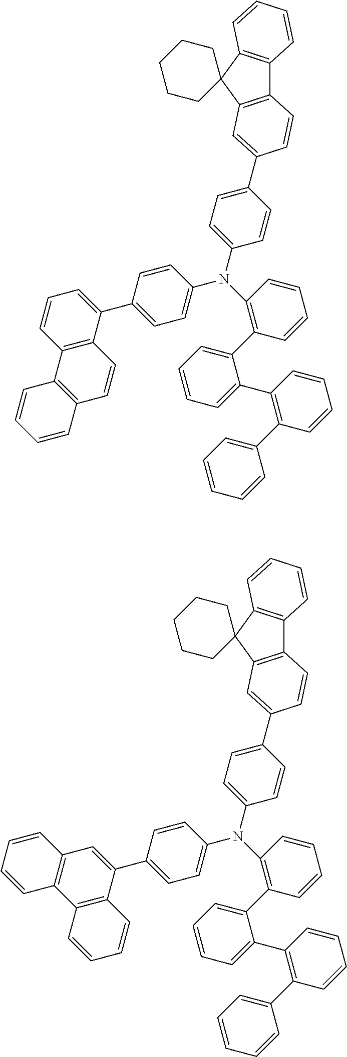 [SMILES: c1ccc(-c2ccccc2-c2ccccc2-c2ccccc2N(c2ccc(-c3ccc4c(c3)C3(CCCCC3)c3ccccc3-4)cc2)c2ccc(-c3cccc4c3ccc3ccccc34)cc2)cc1 ; c1ccc(-c2ccccc2-c2ccccc2-c2ccccc2N(c2ccc(-c3ccc4c(c3)C3(CCCCC3)c3ccccc3-4)cc2)c2ccc(-c3cc4ccccc4c4ccccc34)cc2)cc1]

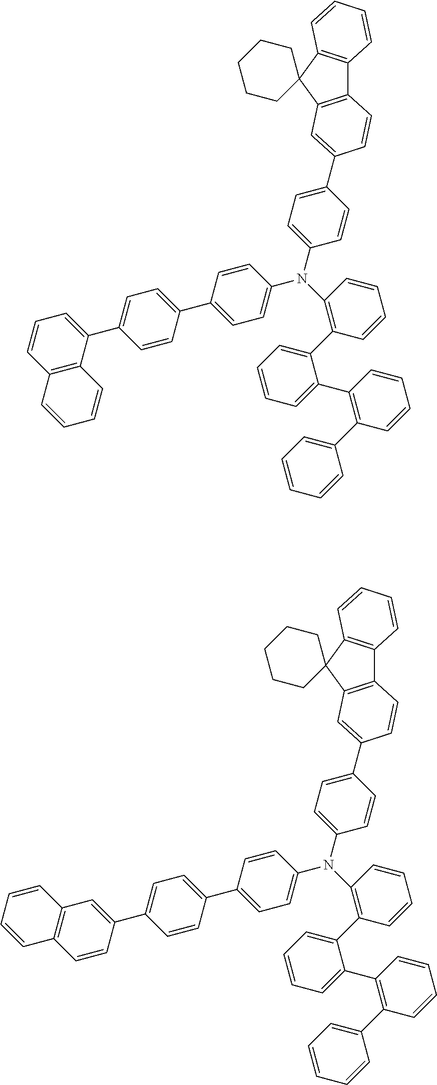 [SMILES: c1ccc(-c2ccccc2-c2ccccc2-c2ccccc2N(c2ccc(-c3ccc(-c4cccc5ccccc45)cc3)cc2)c2ccc(-c3ccc4c(c3)C3(CCCCC3)c3ccccc3-4)cc2)cc1 ; c1ccc(-c2ccccc2-c2ccccc2-c2ccccc2N(c2ccc(-c3ccc(-c4ccc5ccccc5c4)cc3)cc2)c2ccc(-c3ccc4c(c3)C3(CCCCC3)c3ccccc3-4)cc2)cc1]

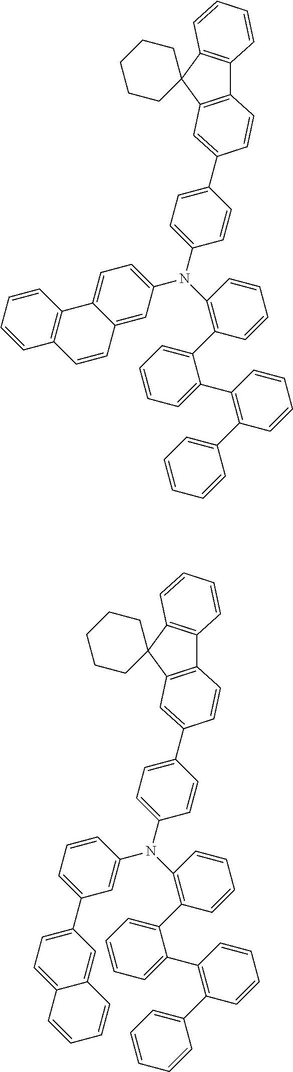 [SMILES: c1ccc(-c2ccccc2-c2ccccc2-c2ccccc2N(c2ccc(-c3ccc4c(c3)C3(CCCCC3)c3ccccc3-4)cc2)c2ccc3c(ccc4ccccc43)c2)cc1 ; c1ccc(-c2ccccc2-c2ccccc2-c2ccccc2N(c2ccc(-c3ccc4c(c3)C3(CCCCC3)c3ccccc3-4)cc2)c2cccc(-c3ccc4ccccc4c3)c2)cc1]

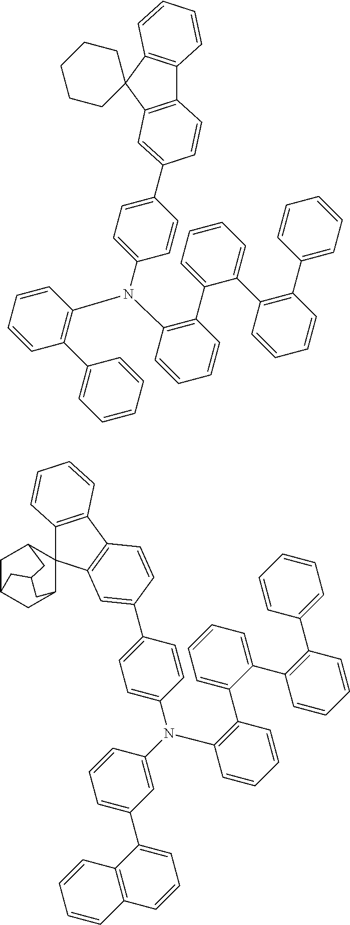 [SMILES: c1ccc(-c2ccccc2-c2ccccc2-c2ccccc2N(c2ccc(-c3ccc4c(c3)C3(CCCCC3)c3ccccc3-4)cc2)c2ccccc2-c2ccccc2)cc1 ; c1ccc(-c2ccccc2-c2ccccc2-c2ccccc2N(c2ccc(-c3ccc4c(c3)C3(c5ccccc5-4)C4CC5CC(C4)CC3C5)cc2)c2cccc(-c3cccc4ccccc34)c2)cc1]

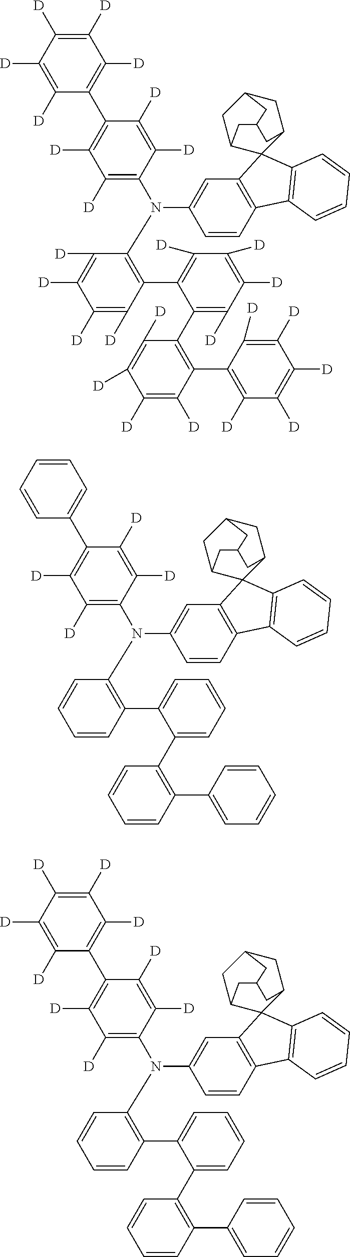

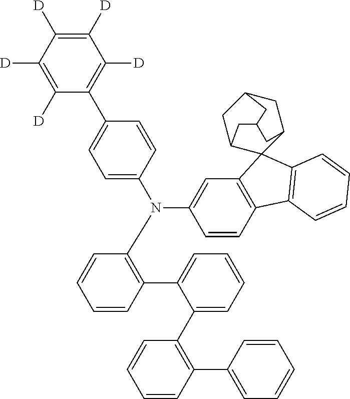 [SMILES: [2H]c1c([2H])c([2H])c(-c2ccc(N(c3ccc4c(c3)C3(c5ccccc5-4)C4CC5CC(C4)CC3C5)c3ccccc3-c3ccccc3-c3ccccc3-c3ccccc3)cc2)c([2H])c1[2H]]

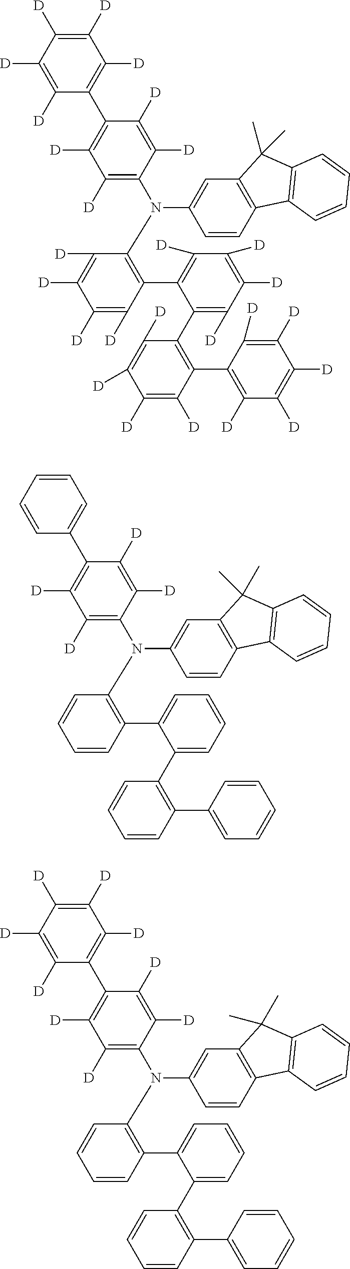

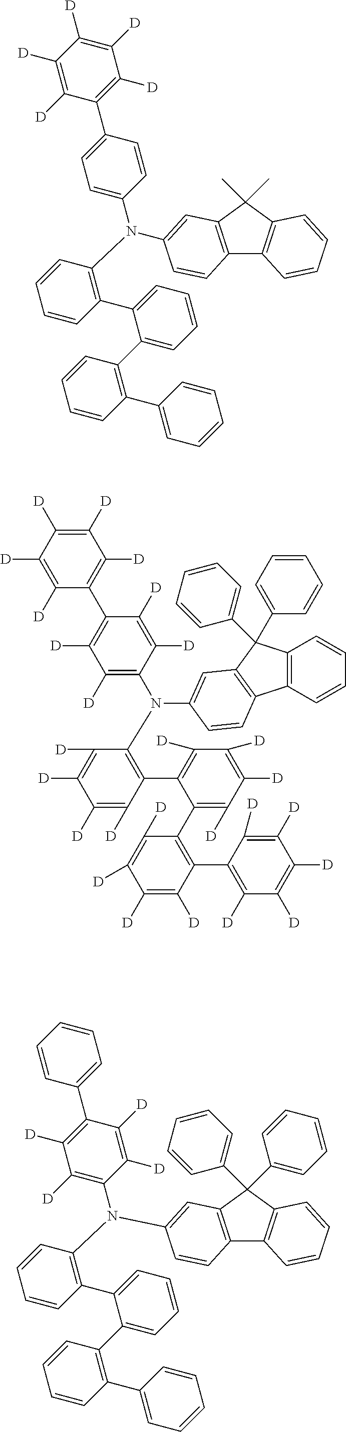 [SMILES: [2H]c1c([2H])c([2H])c(-c2ccc(N(c3ccc4c(c3)C(C)(C)c3ccccc3-4)c3ccccc3-c3ccccc3-c3ccccc3-c3ccccc3)cc2)c([2H])c1[2H] ; [2H]c1c([2H])c([2H])c(-c2c([2H])c([2H])c(N(c3ccc4c(c3)C(c3ccccc3)(c3ccccc3)c3ccccc3-4)c3c([2H])c([2H])c([2H])c([2H])c3-c3c([2H])c([2H])c([2H])c([2H])c3-c3c([2H])c([2H])c([2H])c([2H])c3-c3c([2H])c([2H])c([2H])c([2H])c3[2H])c([2H])c2[2H])c([2H])c1[2H] ; [2H]c1c([2H])c(N(c2ccc3c(c2)C(c2ccccc2)(c2ccccc2)c2ccccc2-3)c2ccccc2-c2ccccc2-c2ccccc2-c2ccccc2)c([2H])c([2H])c1-c1ccccc1]

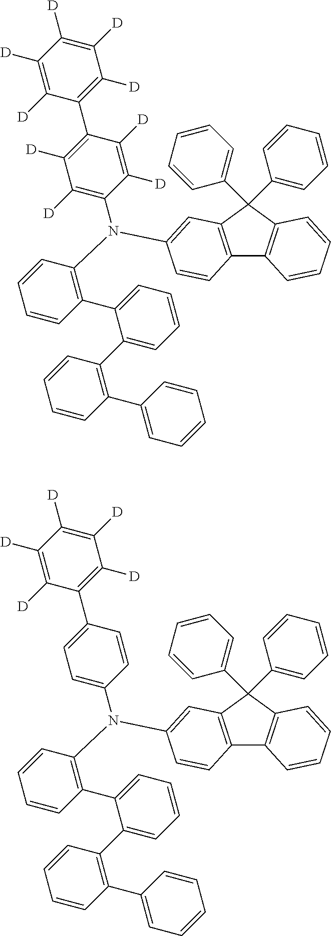 [SMILES: [2H]c1c([2H])c([2H])c(-c2c([2H])c([2H])c(N(c3ccc4c(c3)C(c3ccccc3)(c3ccccc3)c3ccccc3-4)c3ccccc3-c3ccccc3-c3ccccc3-c3ccccc3)c([2H])c2[2H])c([2H])c1[2H] ; [2H]c1c([2H])c([2H])c(-c2ccc(N(c3ccc4c(c3)C(c3ccccc3)(c3ccccc3)c3ccccc3-4)c3ccccc3-c3ccccc3-c3ccccc3-c3ccccc3)cc2)c([2H])c1[2H]]

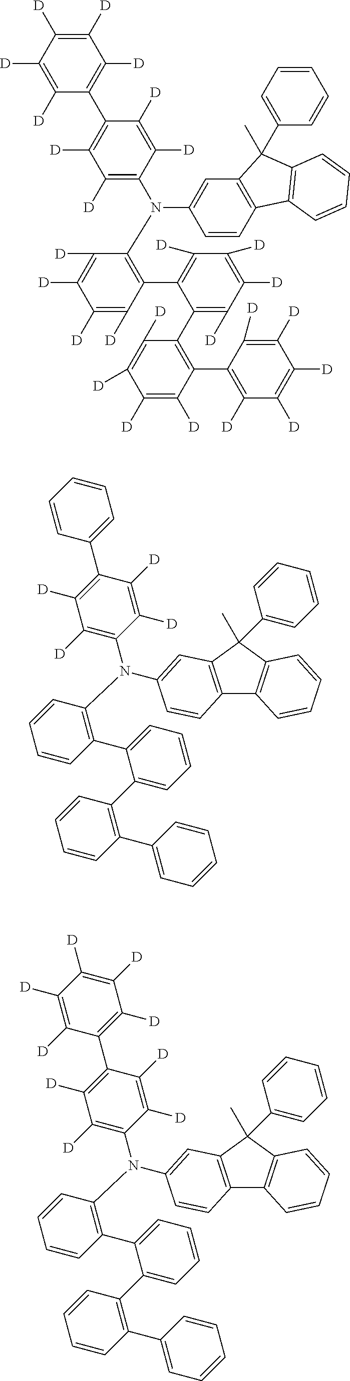

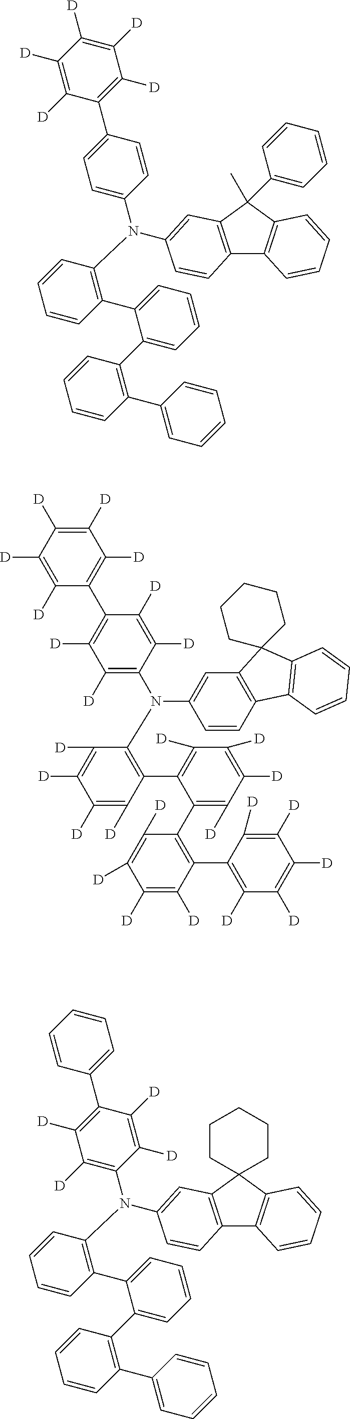 [SMILES: [2H]c1c([2H])c([2H])c(-c2ccc(N(c3ccc4c(c3)C(C)(c3ccccc3)c3ccccc3-4)c3ccccc3-c3ccccc3-c3ccccc3-c3ccccc3)cc2)c([2H])c1[2H] ; [2H]c1c([2H])c([2H])c(-c2c([2H])c([2H])c(N(c3ccc4c(c3)C3(CCCCC3)c3ccccc3-4)c3c([2H])c([2H])c([2H])c([2H])c3-c3c([2H])c([2H])c([2H])c([2H])c3-c3c([2H])c([2H])c([2H])c([2H])c3-c3c([2H])c([2H])c([2H])c([2H])c3[2H])c([2H])c2[2H])c([2H])c1[2H] ; [2H]c1c([2H])c(N(c2ccc3c(c2)C2(CCCCC2)c2ccccc2-3)c2ccccc2-c2ccccc2-c2ccccc2-c2ccccc2)c([2H])c([2H])c1-c1ccccc1]

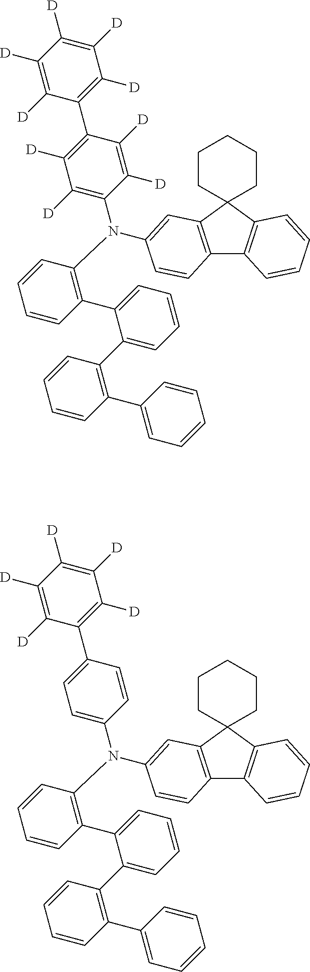 [SMILES: [2H]c1c([2H])c([2H])c(-c2c([2H])c([2H])c(N(c3ccc4c(c3)C3(CCCCC3)c3ccccc3-4)c3ccccc3-c3ccccc3-c3ccccc3-c3ccccc3)c([2H])c2[2H])c([2H])c1[2H] ; [2H]c1c([2H])c([2H])c(-c2ccc(N(c3ccc4c(c3)C3(CCCCC3)c3ccccc3-4)c3ccccc3-c3ccccc3-c3ccccc3-c3ccccc3)cc2)c([2H])c1[2H]]

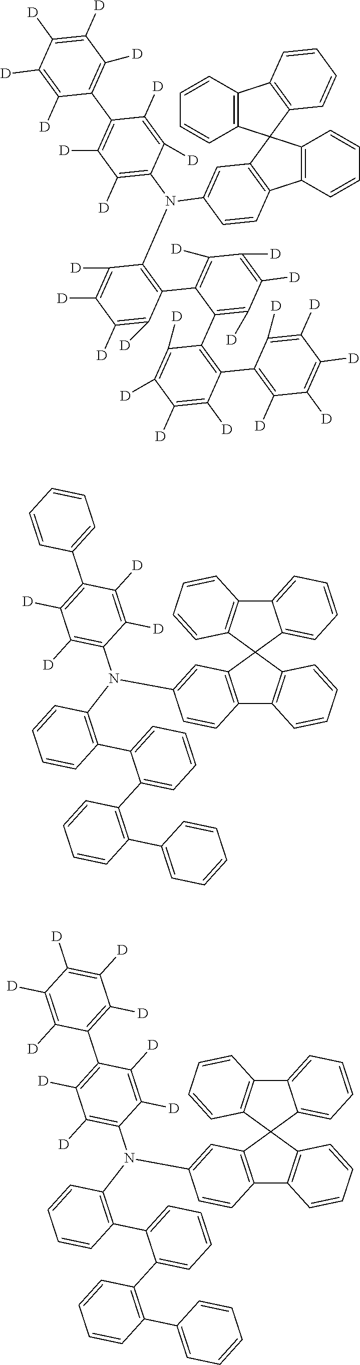

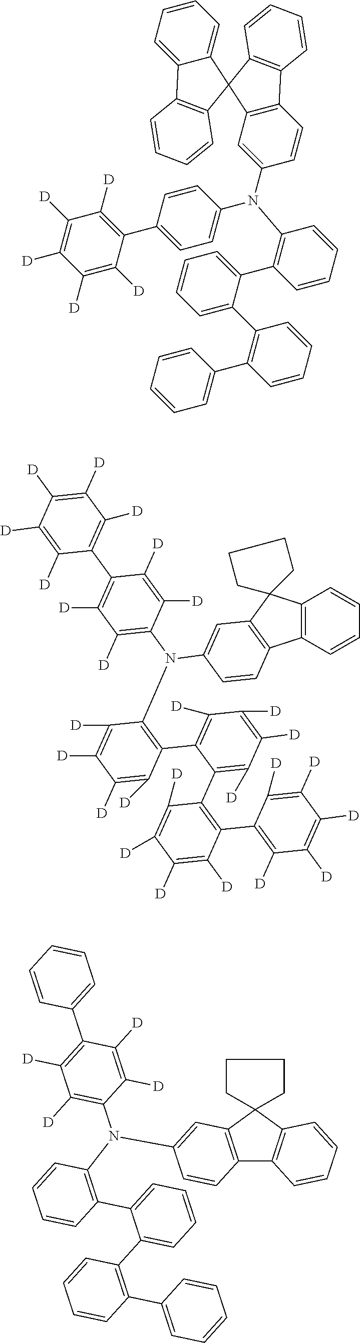 [SMILES: [2H]c1c([2H])c([2H])c(-c2ccc(N(c3ccc4c(c3)C3(c5ccccc5-c5ccccc53)c3ccccc3-4)c3ccccc3-c3ccccc3-c3ccccc3-c3ccccc3)cc2)c([2H])c1[2H] ; [2H]c1c([2H])c([2H])c(-c2c([2H])c([2H])c(N(c3ccc4c(c3)C3(CCCC3)c3ccccc3-4)c3c([2H])c([2H])c([2H])c([2H])c3-c3c([2H])c([2H])c([2H])c([2H])c3-c3c([2H])c([2H])c([2H])c([2H])c3-c3c([2H])c([2H])c([2H])c([2H])c3[2H])c([2H])c2[2H])c([2H])c1[2H] ; [2H]c1c([2H])c(N(c2ccc3c(c2)C2(CCCC2)c2ccccc2-3)c2ccccc2-c2ccccc2-c2ccccc2-c2ccccc2)c([2H])c([2H])c1-c1ccccc1]

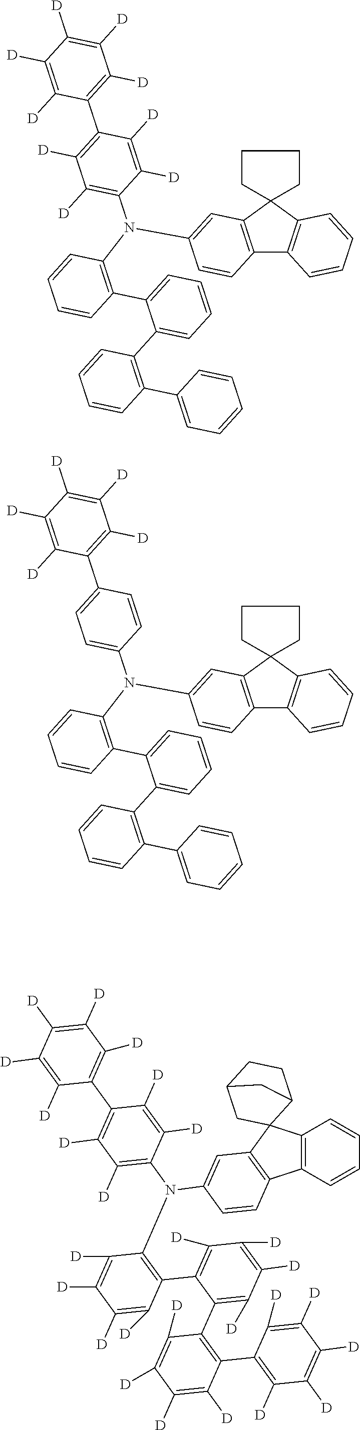 [SMILES: [2H]c1c([2H])c([2H])c(-c2c([2H])c([2H])c(N(c3ccc4c(c3)C3(CCCC3)c3ccccc3-4)c3ccccc3-c3ccccc3-c3ccccc3-c3ccccc3)c([2H])c2[2H])c([2H])c1[2H] ; [2H]c1c([2H])c([2H])c(-c2ccc(N(c3ccc4c(c3)C3(CCCC3)c3ccccc3-4)c3ccccc3-c3ccccc3-c3ccccc3-c3ccccc3)cc2)c([2H])c1[2H] ; [2H]c1c([2H])c([2H])c(-c2c([2H])c([2H])c(N(c3ccc4c(c3)C3(CC5CCC3C5)c3ccccc3-4)c3c([2H])c([2H])c([2H])c([2H])c3-c3c([2H])c([2H])c([2H])c([2H])c3-c3c([2H])c([2H])c([2H])c([2H])c3-c3c([2H])c([2H])c([2H])c([2H])c3[2H])c([2H])c2[2H])c([2H])c1[2H]]

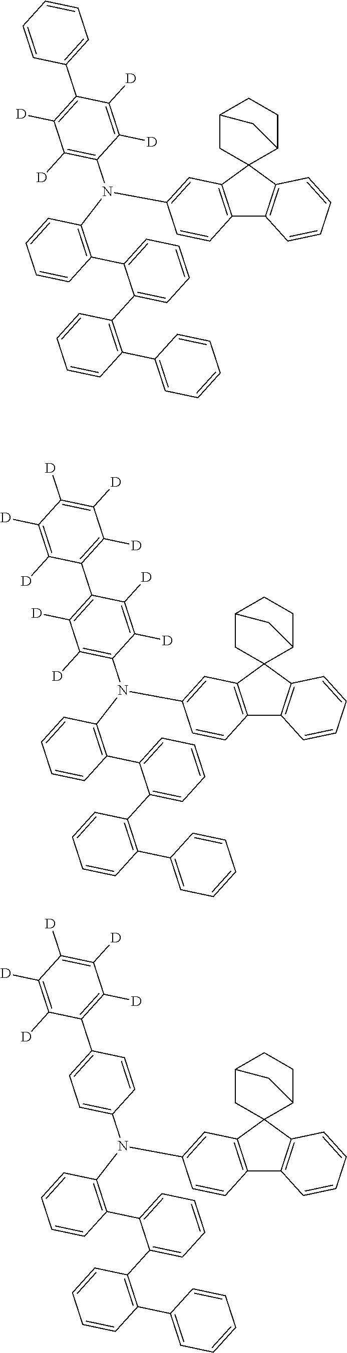 [SMILES: [2H]c1c([2H])c(N(c2ccc3c(c2)C2(CC4CCC2C4)c2ccccc2-3)c2ccccc2-c2ccccc2-c2ccccc2-c2ccccc2)c([2H])c([2H])c1-c1ccccc1 ; [2H]c1c([2H])c([2H])c(-c2c([2H])c([2H])c(N(c3ccc4c(c3)C3(CC5CCC3C5)c3ccccc3-4)c3ccccc3-c3ccccc3-c3ccccc3-c3ccccc3)c([2H])c2[2H])c([2H])c1[2H] ; [2H]c1c([2H])c([2H])c(-c2ccc(N(c3ccc4c(c3)C3(CC5CCC3C5)c3ccccc3-4)c3ccccc3-c3ccccc3-c3ccccc3-c3ccccc3)cc2)c([2H])c1[2H]]

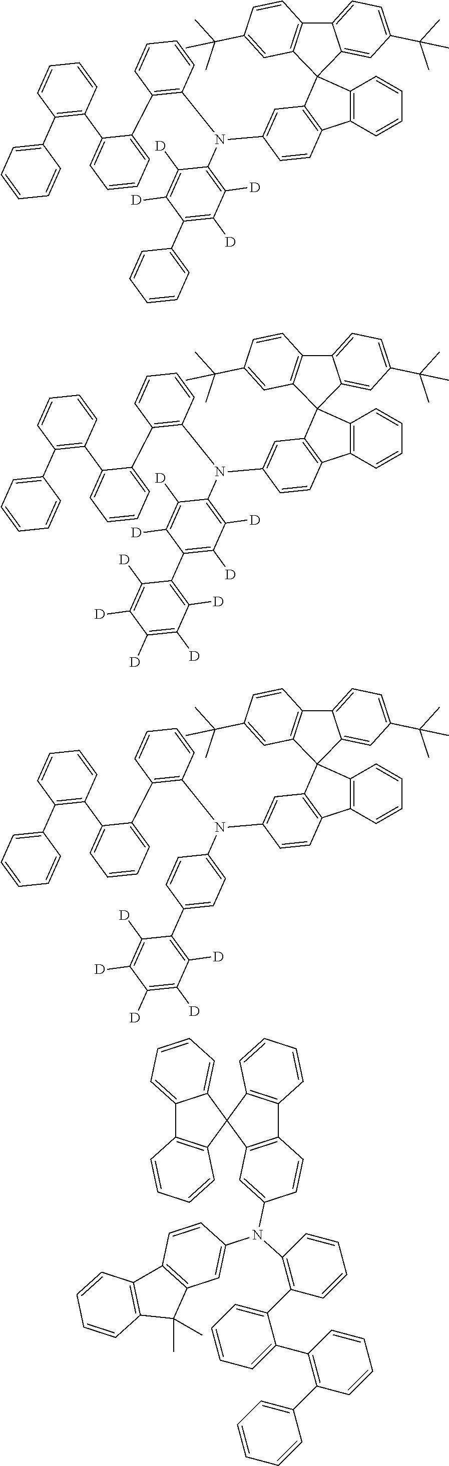

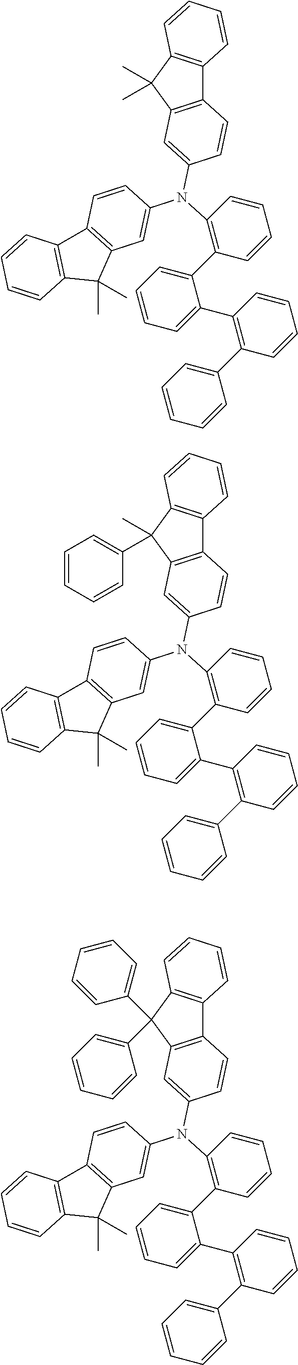

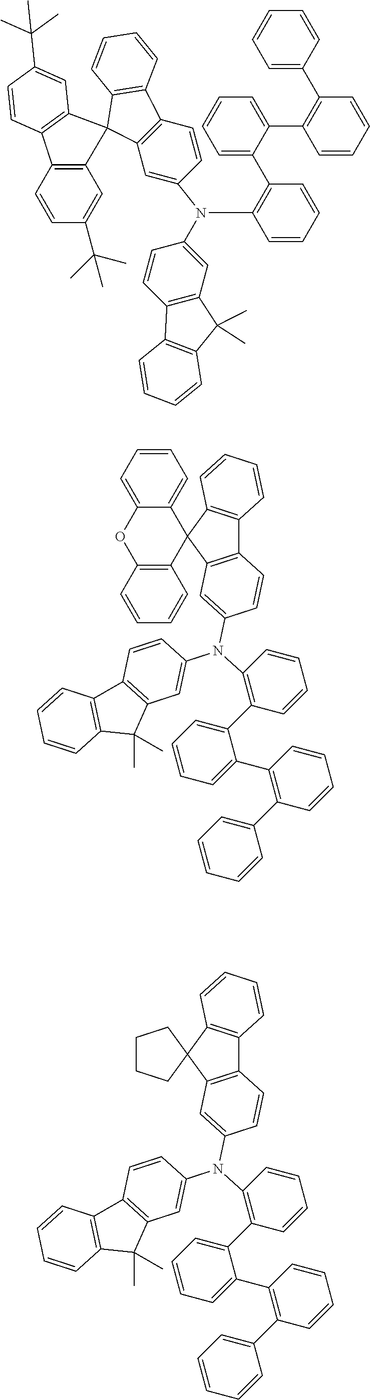

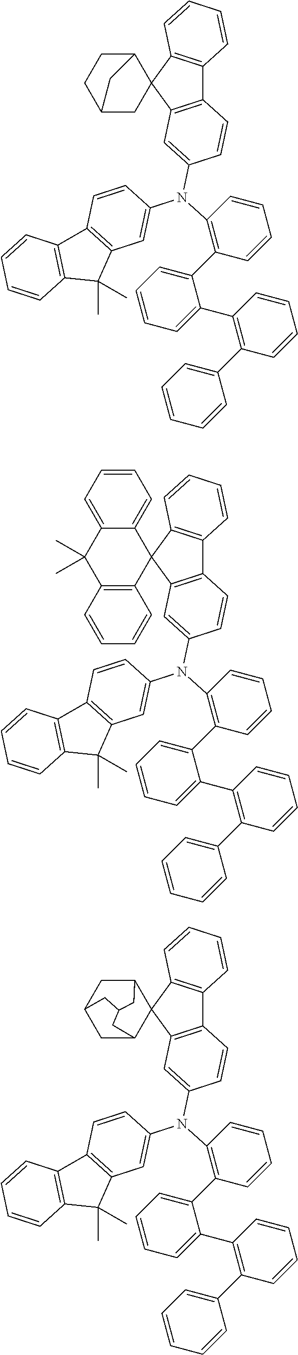

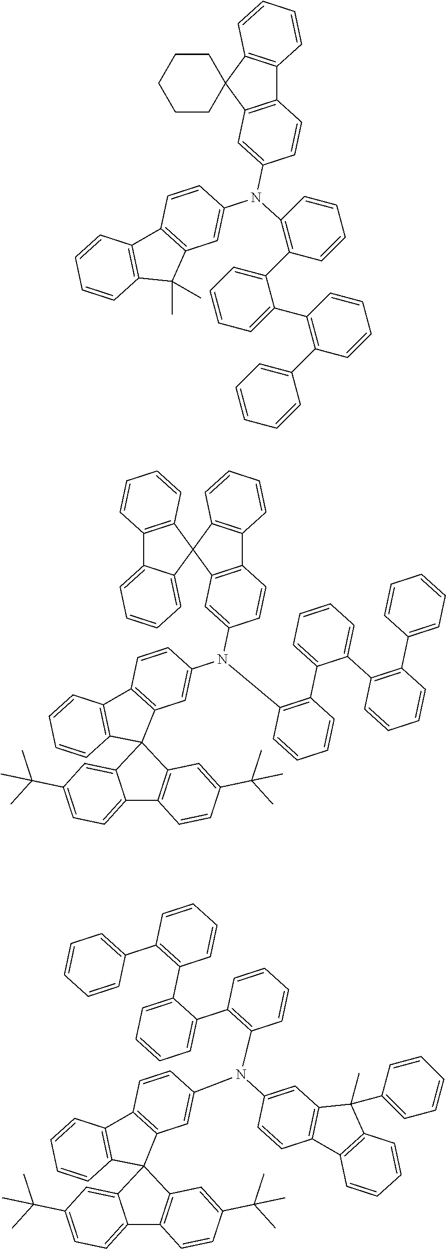 [SMILES: CC1(C)c2ccccc2-c2ccc(N(c3ccc4c(c3)C3(CCCCC3)c3ccccc3-4)c3ccccc3-c3ccccc3-c3ccccc3-c3ccccc3)cc21 ; CC(C)(C)c1ccc2c(c1)C1(c3ccccc3-c3ccc(N(c4ccc5c(c4)C4(c6ccccc6-c6ccccc64)c4ccccc4-5)c4ccccc4-c4ccccc4-c4ccccc4-c4ccccc4)cc31)c1cc(C(C)(C)C)ccc1-2 ; CC(C)(C)c1ccc2c(c1)C1(c3ccccc3-c3ccc(N(c4ccc5c(c4)C(C)(c4ccccc4)c4ccccc4-5)c4ccccc4-c4ccccc4-c4ccccc4-c4ccccc4)cc31)c1cc(C(C)(C)C)ccc1-2]

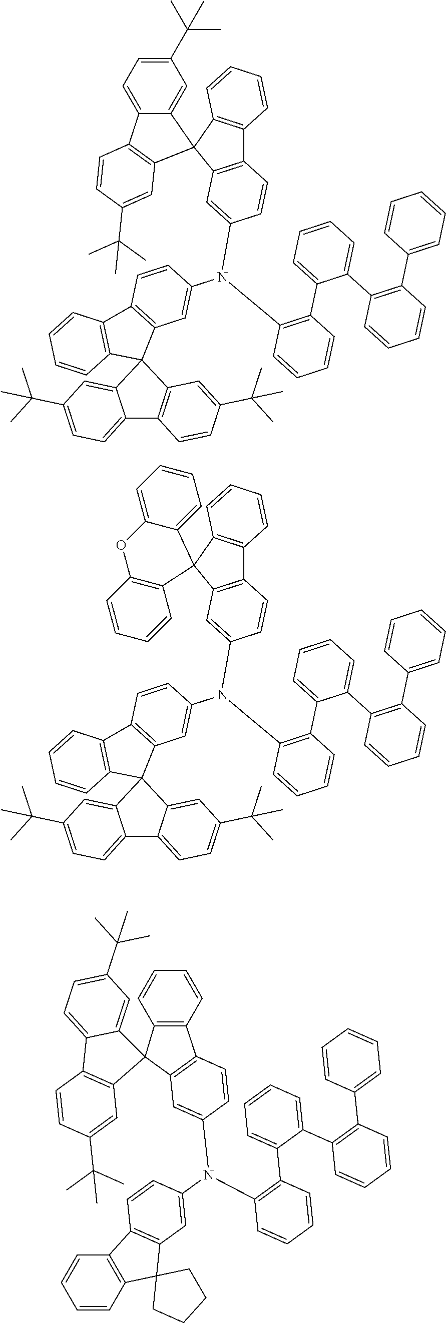

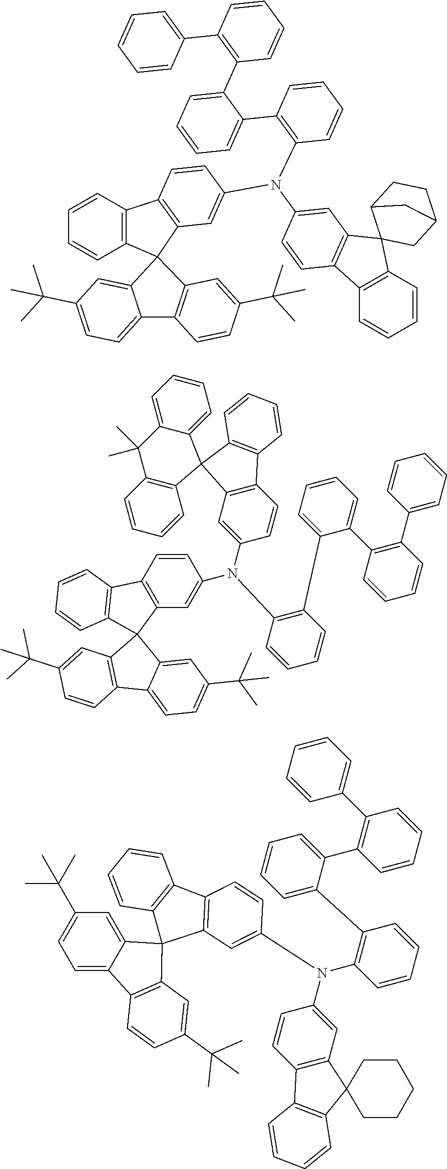

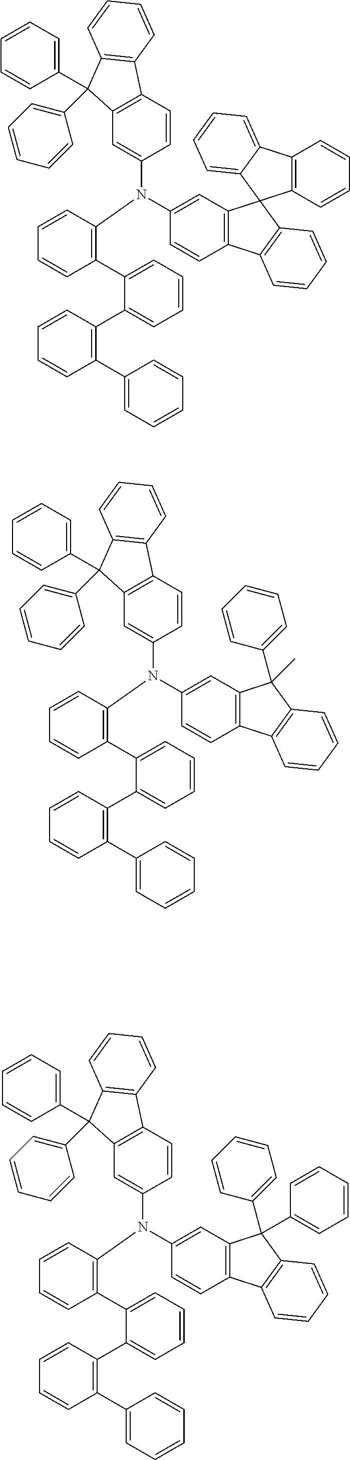 [SMILES: c1ccc(-c2ccccc2-c2ccccc2-c2ccccc2N(c2ccc3c(c2)C(c2ccccc2)(c2ccccc2)c2ccccc2-3)c2ccc3c(c2)C2(c4ccccc4-c4ccccc42)c2ccccc2-3)cc1 ; CC1(c2ccccc2)c2ccccc2-c2ccc(N(c3ccc4c(c3)C(c3ccccc3)(c3ccccc3)c3ccccc3-4)c3ccccc3-c3ccccc3-c3ccccc3-c3ccccc3)cc21 ; c1ccc(-c2ccccc2-c2ccccc2-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1]

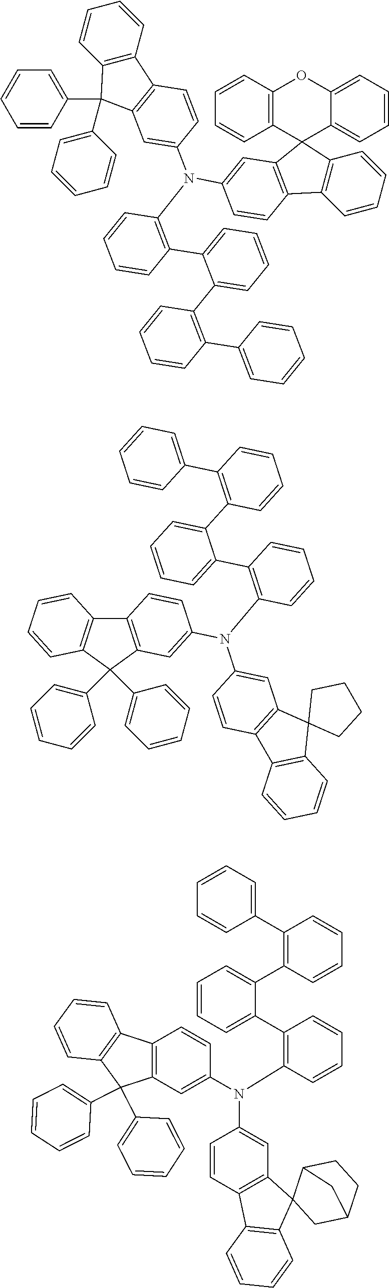 [SMILES: c1ccc(-c2ccccc2-c2ccccc2-c2ccccc2N(c2ccc3c(c2)C(c2ccccc2)(c2ccccc2)c2ccccc2-3)c2ccc3c(c2)C2(c4ccccc4Oc4ccccc42)c2ccccc2-3)cc1 ; c1ccc(-c2ccccc2-c2ccccc2-c2ccccc2N(c2ccc3c(c2)C2(CCCC2)c2ccccc2-3)c2ccc3c(c2)C(c2ccccc2)(c2ccccc2)c2ccccc2-3)cc1 ; c1ccc(-c2ccccc2-c2ccccc2-c2ccccc2N(c2ccc3c(c2)C(c2ccccc2)(c2ccccc2)c2ccccc2-3)c2ccc3c(c2)C2(CC4CCC2C4)c2ccccc2-3)cc1]

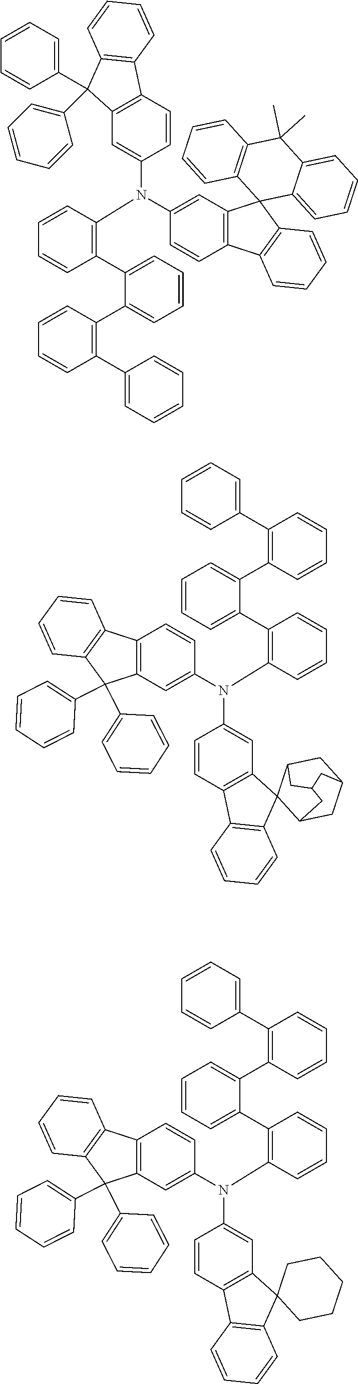 [SMILES: CC1(C)c2ccccc2C2(c3ccccc3-c3ccc(N(c4ccc5c(c4)C(c4ccccc4)(c4ccccc4)c4ccccc4-5)c4ccccc4-c4ccccc4-c4ccccc4-c4ccccc4)cc32)c2ccccc21 ; c1ccc(-c2ccccc2-c2ccccc2-c2ccccc2N(c2ccc3c(c2)C(c2ccccc2)(c2ccccc2)c2ccccc2-3)c2ccc3c(c2)C2(c4ccccc4-3)C3CC4CC(C3)CC2C4)cc1 ; c1ccc(-c2ccccc2-c2ccccc2-c2ccccc2N(c2ccc3c(c2)C2(CCCCC2)c2ccccc2-3)c2ccc3c(c2)C(c2ccccc2)(c2ccccc2)c2ccccc2-3)cc1]

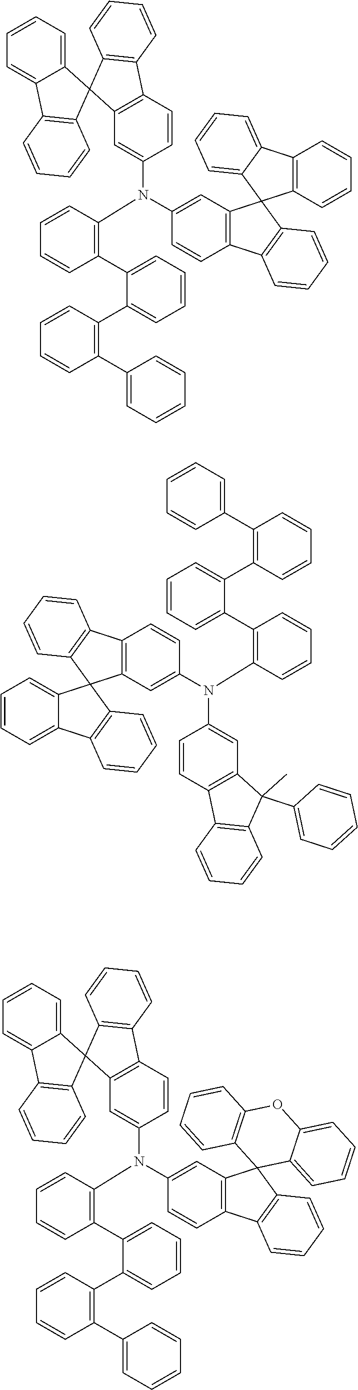 [SMILES: c1ccc(-c2ccccc2-c2ccccc2-c2ccccc2N(c2ccc3c(c2)C2(c4ccccc4-c4ccccc42)c2ccccc2-3)c2ccc3c(c2)C2(c4ccccc4-c4ccccc42)c2ccccc2-3)cc1 ; CC1(c2ccccc2)c2ccccc2-c2ccc(N(c3ccc4c(c3)C3(c5ccccc5-c5ccccc53)c3ccccc3-4)c3ccccc3-c3ccccc3-c3ccccc3-c3ccccc3)cc21 ; c1ccc(-c2ccccc2-c2ccccc2-c2ccccc2N(c2ccc3c(c2)C2(c4ccccc4Oc4ccccc42)c2ccccc2-3)c2ccc3c(c2)C2(c4ccccc4-c4ccccc42)c2ccccc2-3)cc1]

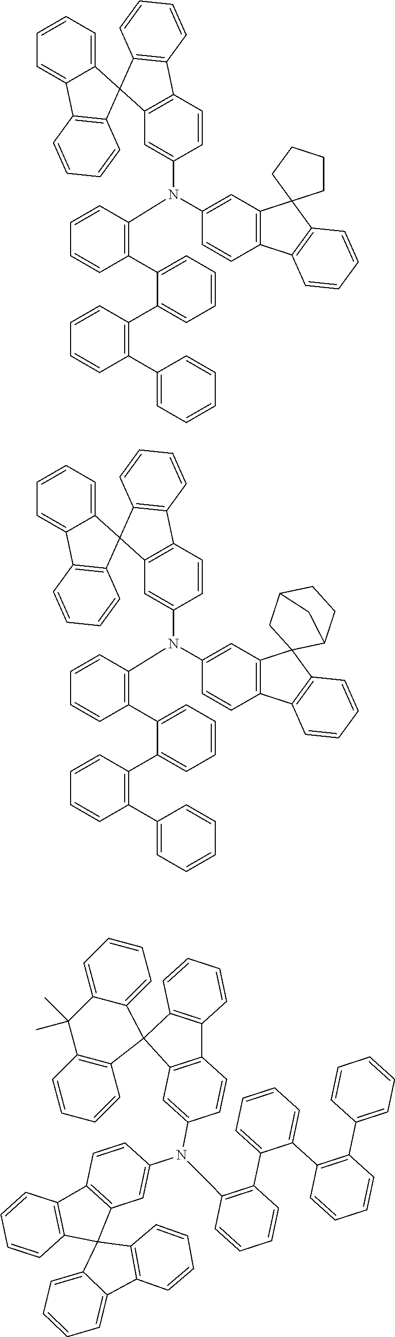

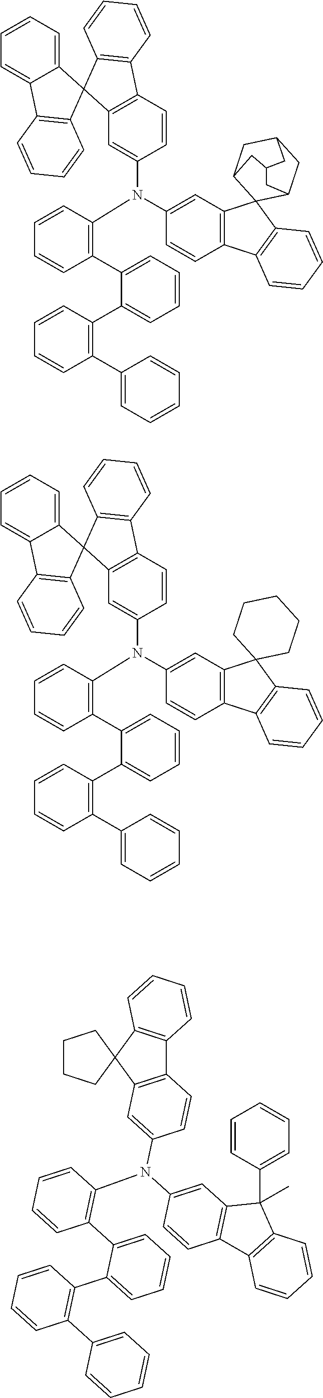

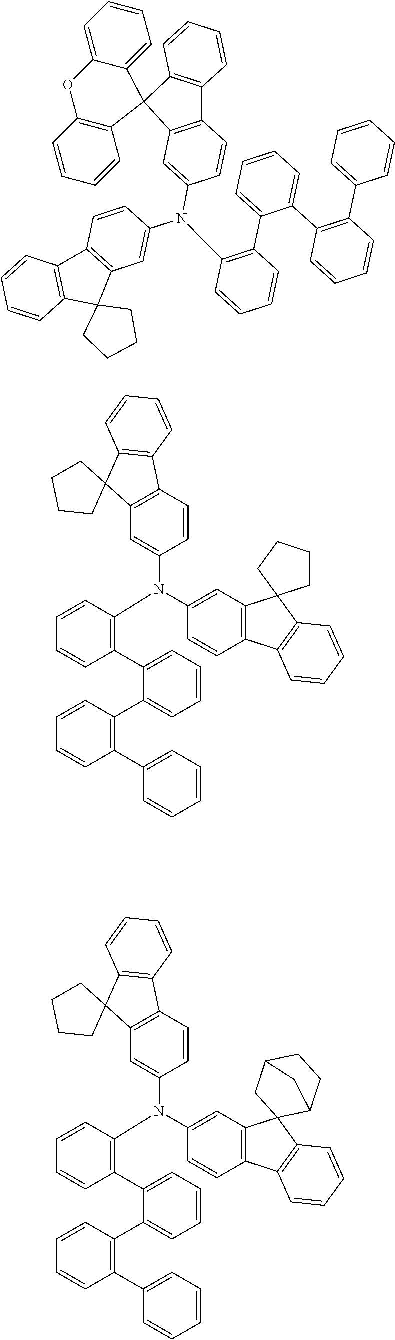

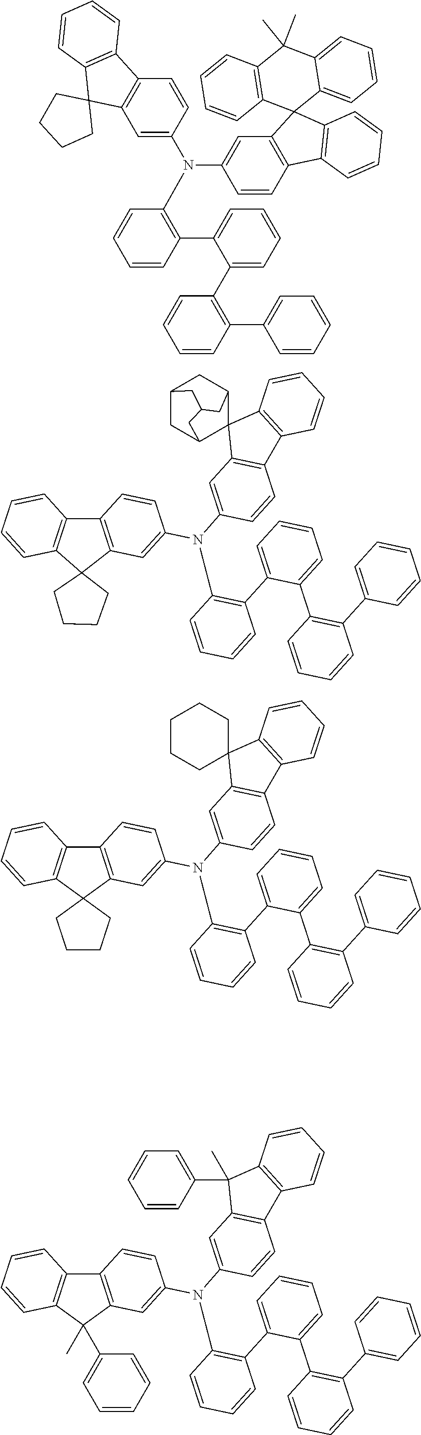

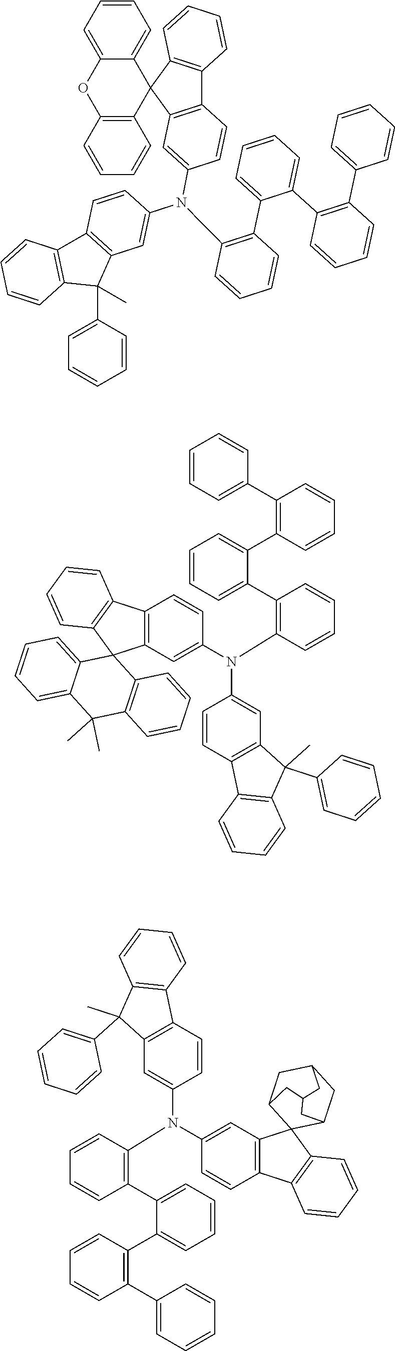 [SMILES: CC1(c2ccccc2)c2ccccc2-c2ccc(N(c3ccc4c(c3)C3(c5ccccc5Oc5ccccc53)c3ccccc3-4)c3ccccc3-c3ccccc3-c3ccccc3-c3ccccc3)cc21 ; CC1(C)c2ccccc2C2(c3ccccc3-c3ccc(N(c4ccc5c(c4)C(C)(c4ccccc4)c4ccccc4-5)c4ccccc4-c4ccccc4-c4ccccc4-c4ccccc4)cc32)c2ccccc21 ; CC1(c2ccccc2)c2ccccc2-c2ccc(N(c3ccc4c(c3)C3(c5ccccc5-4)C4CC5CC(C4)CC3C5)c3ccccc3-c3ccccc3-c3ccccc3-c3ccccc3)cc21]

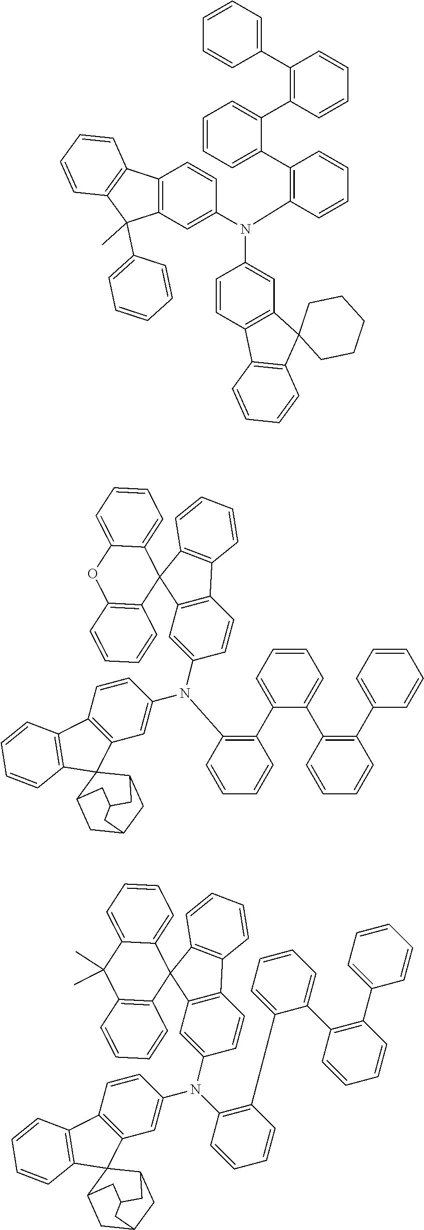 [SMILES: CC1(c2ccccc2)c2ccccc2-c2ccc(N(c3ccc4c(c3)C3(CCCCC3)c3ccccc3-4)c3ccccc3-c3ccccc3-c3ccccc3-c3ccccc3)cc21 ; c1ccc(-c2ccccc2-c2ccccc2-c2ccccc2N(c2ccc3c(c2)C2(c4ccccc4Oc4ccccc42)c2ccccc2-3)c2ccc3c(c2)C2(c4ccccc4-3)C3CC4CC(C3)CC2C4)cc1 ; CC1(C)c2ccccc2C2(c3ccccc3-c3ccc(N(c4ccc5c(c4)C4(c6ccccc6-5)C5CC6CC(C5)CC4C6)c4ccccc4-c4ccccc4-c4ccccc4-c4ccccc4)cc32)c2ccccc21]

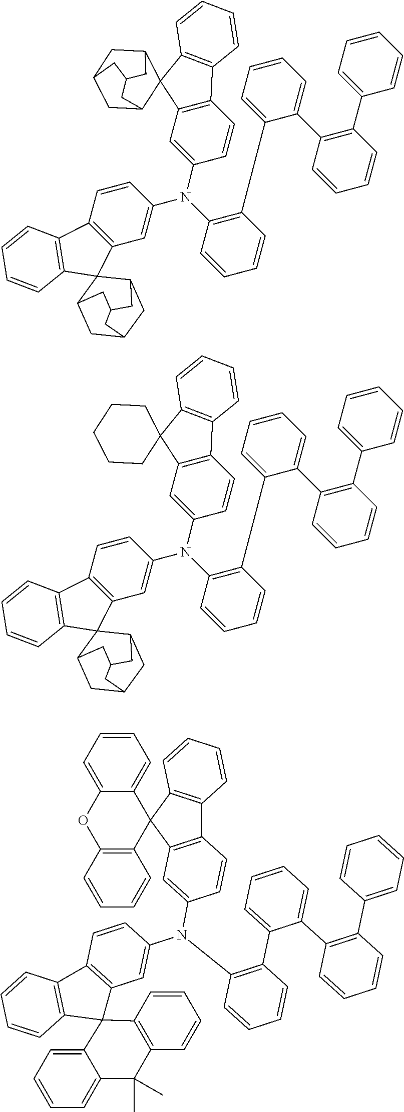

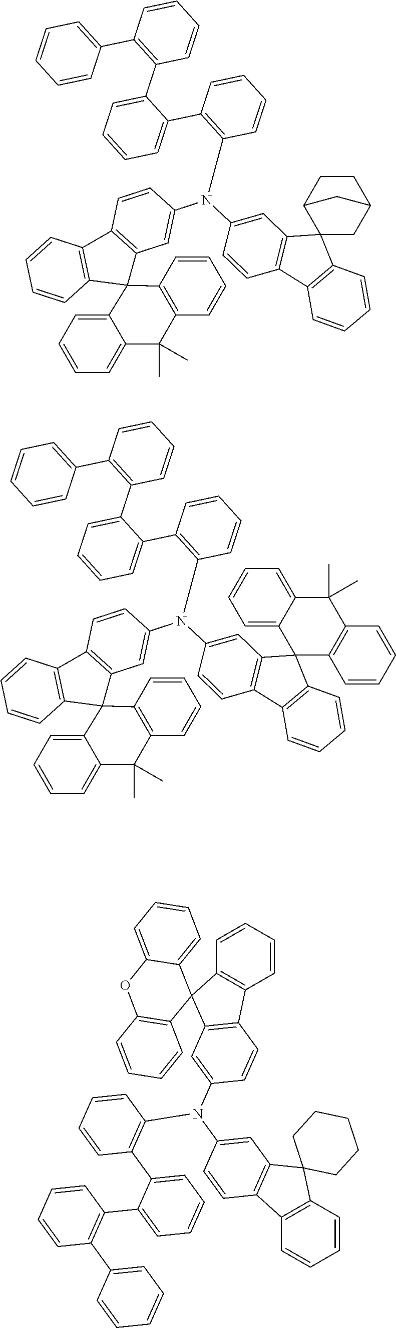

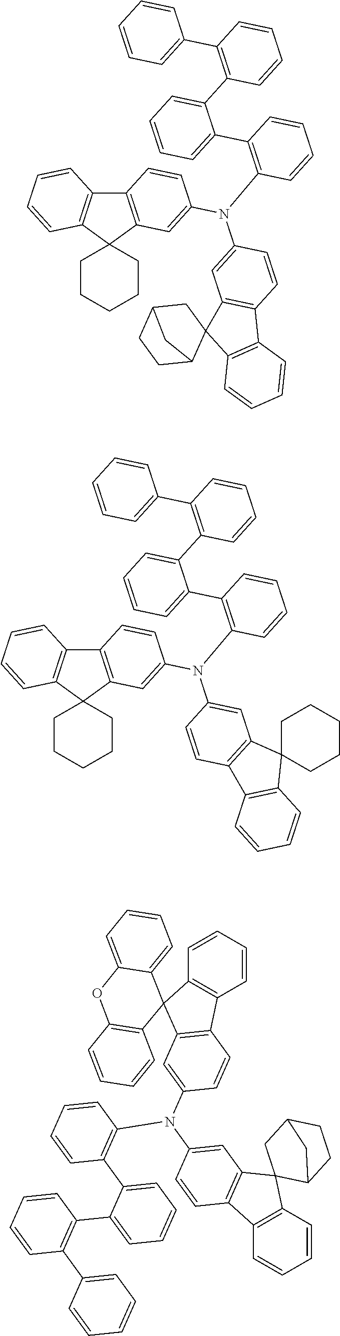 [SMILES: c1ccc(-c2ccccc2-c2ccccc2-c2ccccc2N(c2ccc3c(c2)C2(CCCCC2)c2ccccc2-3)c2ccc3c(c2)C2(CC4CCC2C4)c2ccccc2-3)cc1 ; c1ccc(-c2ccccc2-c2ccccc2-c2ccccc2N(c2ccc3c(c2)C2(CCCCC2)c2ccccc2-3)c2ccc3c(c2)C2(CCCCC2)c2ccccc2-3)cc1 ; c1ccc(-c2ccccc2-c2ccccc2-c2ccccc2N(c2ccc3c(c2)C2(c4ccccc4Oc4ccccc42)c2ccccc2-3)c2ccc3c(c2)C2(CC4CCC2C4)c2ccccc2-3)cc1]

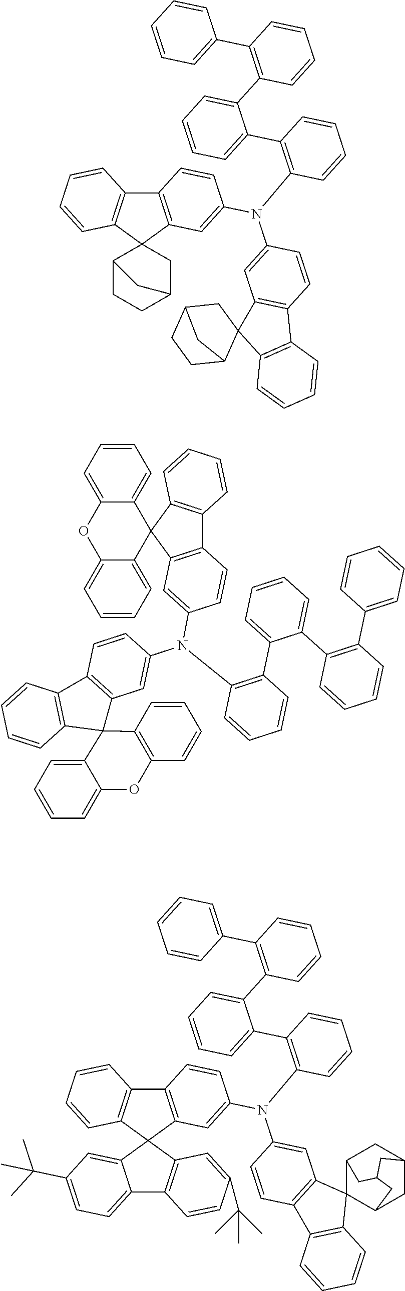 [SMILES: c1ccc(-c2ccccc2-c2ccccc2-c2ccccc2N(c2ccc3c(c2)C2(CC4CCC2C4)c2ccccc2-3)c2ccc3c(c2)C2(CC4CCC2C4)c2ccccc2-3)cc1 ; c1ccc(-c2ccccc2-c2ccccc2-c2ccccc2N(c2ccc3c(c2)C2(c4ccccc4Oc4ccccc42)c2ccccc2-3)c2ccc3c(c2)C2(c4ccccc4Oc4ccccc42)c2ccccc2-3)cc1 ; CC(C)(C)c1ccc2c(c1)C1(c3ccccc3-c3ccc(N(c4ccc5c(c4)C4(c6ccccc6-5)C5CC6CC(C5)CC4C6)c4ccccc4-c4ccccc4-c4ccccc4-c4ccccc4)cc31)c1cc(C(C)(C)C)ccc1-2]

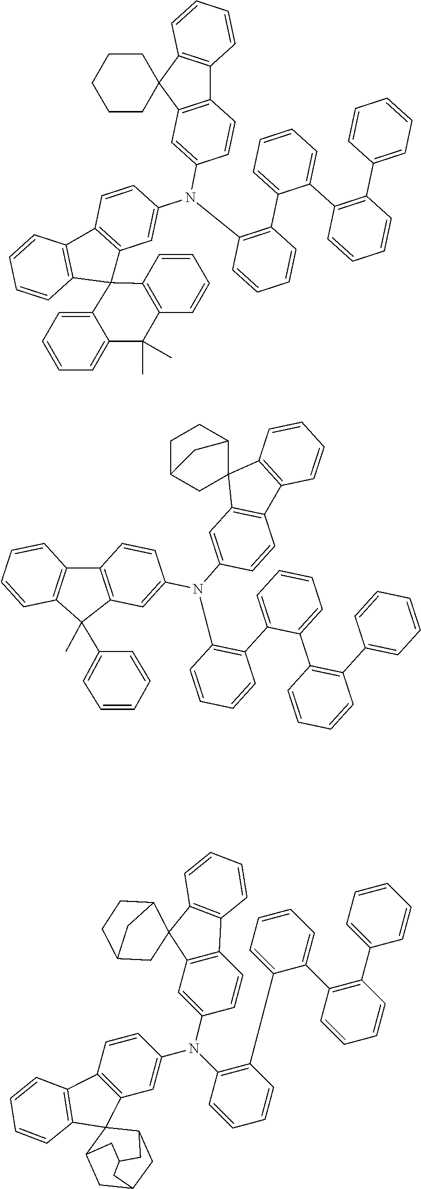 [SMILES: CC1(C)c2ccccc2C2(c3ccccc3-c3ccc(N(c4ccc5c(c4)C4(CCCCC4)c4ccccc4-5)c4ccccc4-c4ccccc4-c4ccccc4-c4ccccc4)cc32)c2ccccc21 ; CC1(c2ccccc2)c2ccccc2-c2ccc(N(c3ccc4c(c3)C3(CC5CCC3C5)c3ccccc3-4)c3ccccc3-c3ccccc3-c3ccccc3-c3ccccc3)cc21 ; c1ccc(-c2ccccc2-c2ccccc2-c2ccccc2N(c2ccc3c(c2)C2(CC4CCC2C4)c2ccccc2-3)c2ccc3c(c2)C2(c4ccccc4-3)C3CC4CC(C3)CC2C4)cc1]

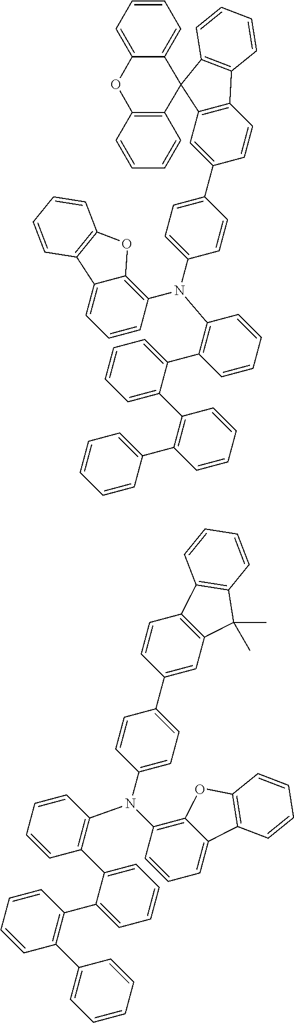 [SMILES: c1ccc(-c2ccccc2-c2ccccc2-c2ccccc2N(c2ccc(-c3ccc4c(c3)C3(c5ccccc5Oc5ccccc53)c3ccccc3-4)cc2)c2cccc3c2oc2ccccc23)cc1 ; CC1(C)c2ccccc2-c2ccc(-c3ccc(N(c4ccccc4-c4ccccc4-c4ccccc4-c4ccccc4)c4cccc5c4oc4ccccc45)cc3)cc21]

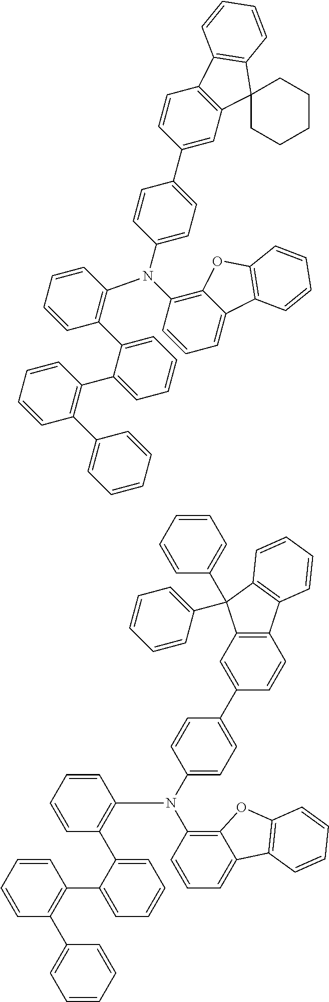

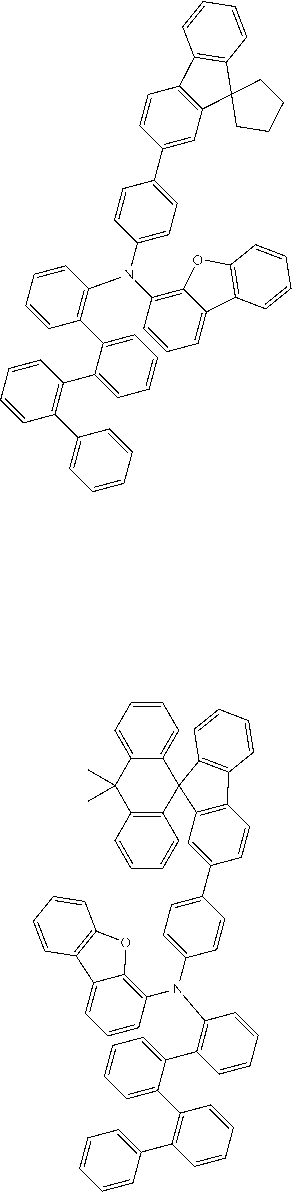 [SMILES: c1ccc(-c2ccccc2-c2ccccc2-c2ccccc2N(c2ccc(-c3ccc4c(c3)C3(CCCC3)c3ccccc3-4)cc2)c2cccc3c2oc2ccccc23)cc1 ; CC1(C)c2ccccc2C2(c3ccccc3-c3ccc(-c4ccc(N(c5ccccc5-c5ccccc5-c5ccccc5-c5ccccc5)c5cccc6c5oc5ccccc56)cc4)cc32)c2ccccc21]

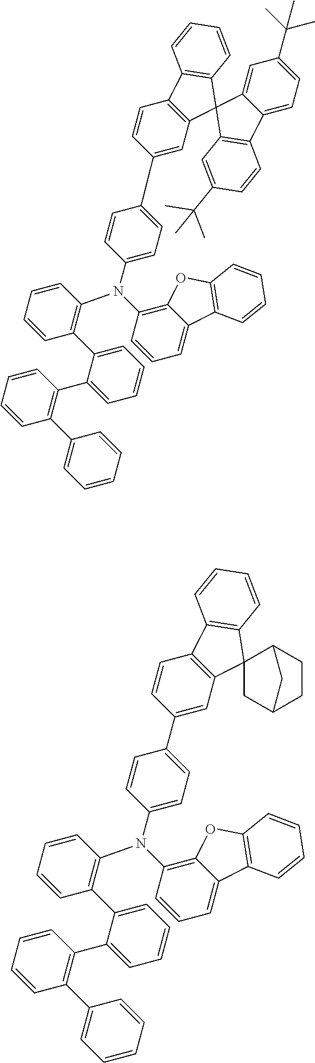 [SMILES: CC(C)(C)c1ccc2c(c1)C1(c3ccccc3-c3ccc(-c4ccc(N(c5ccccc5-c5ccccc5-c5ccccc5-c5ccccc5)c5cccc6c5oc5ccccc56)cc4)cc31)c1cc(C(C)(C)C)ccc1-2 ; c1ccc(-c2ccccc2-c2ccccc2-c2ccccc2N(c2ccc(-c3ccc4c(c3)C3(CC5CCC3C5)c3ccccc3-4)cc2)c2cccc3c2oc2ccccc23)cc1]

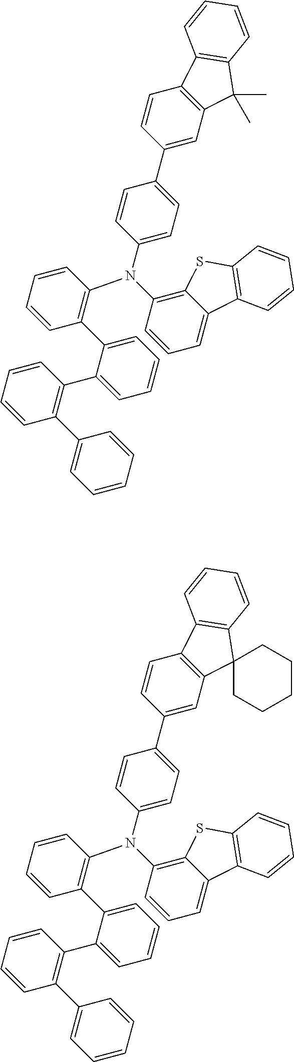 [SMILES: CC1(C)c2ccccc2-c2ccc(-c3ccc(N(c4ccccc4-c4ccccc4-c4ccccc4-c4ccccc4)c4cccc5c4sc4ccccc45)cc3)cc21 ; c1ccc(-c2ccccc2-c2ccccc2-c2ccccc2N(c2ccc(-c3ccc4c(c3)C3(CCCCC3)c3ccccc3-4)cc2)c2cccc3c2sc2ccccc23)cc1]

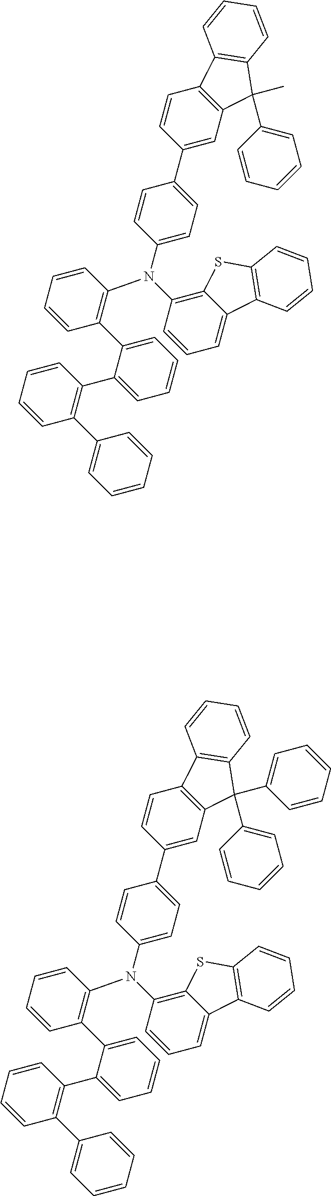 [SMILES: CC1(c2ccccc2)c2ccccc2-c2ccc(-c3ccc(N(c4ccccc4-c4ccccc4-c4ccccc4-c4ccccc4)c4cccc5c4sc4ccccc45)cc3)cc21 ; c1ccc(-c2ccccc2-c2ccccc2-c2ccccc2N(c2ccc(-c3ccc4c(c3)C(c3ccccc3)(c3ccccc3)c3ccccc3-4)cc2)c2cccc3c2sc2ccccc23)cc1]

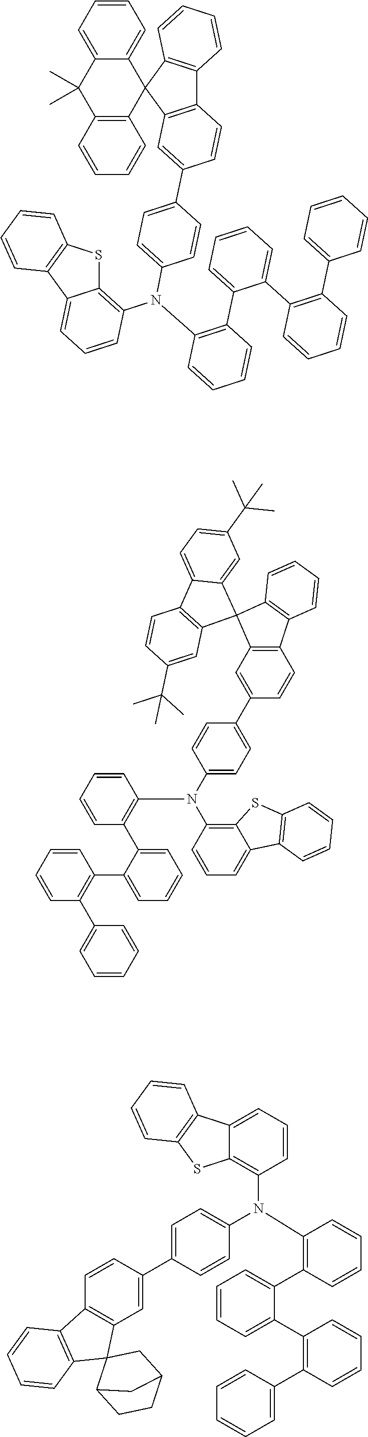 [SMILES: CC1(C)c2ccccc2C2(c3ccccc3-c3ccc(-c4ccc(N(c5ccccc5-c5ccccc5-c5ccccc5-c5ccccc5)c5cccc6c5sc5ccccc56)cc4)cc32)c2ccccc21 ; CC(C)(C)c1ccc2c(c1)C1(c3ccccc3-c3ccc(-c4ccc(N(c5ccccc5-c5ccccc5-c5ccccc5-c5ccccc5)c5cccc6c5sc5ccccc56)cc4)cc31)c1cc(C(C)(C)C)ccc1-2 ; c1ccc(-c2ccccc2-c2ccccc2-c2ccccc2N(c2ccc(-c3ccc4c(c3)C3(CC5CCC3C5)c3ccccc3-4)cc2)c2cccc3c2sc2ccccc23)cc1]

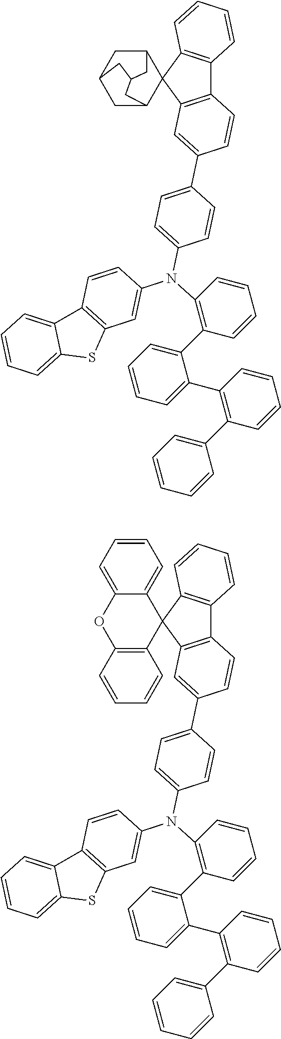 [SMILES: c1ccc(-c2ccccc2-c2ccccc2-c2ccccc2N(c2ccc(-c3ccc4c(c3)C3(c5ccccc5-4)C4CC5CC(C4)CC3C5)cc2)c2ccc3c(c2)sc2ccccc23)cc1 ; c1ccc(-c2ccccc2-c2ccccc2-c2ccccc2N(c2ccc(-c3ccc4c(c3)C3(c5ccccc5Oc5ccccc53)c3ccccc3-4)cc2)c2ccc3c(c2)sc2ccccc23)cc1]

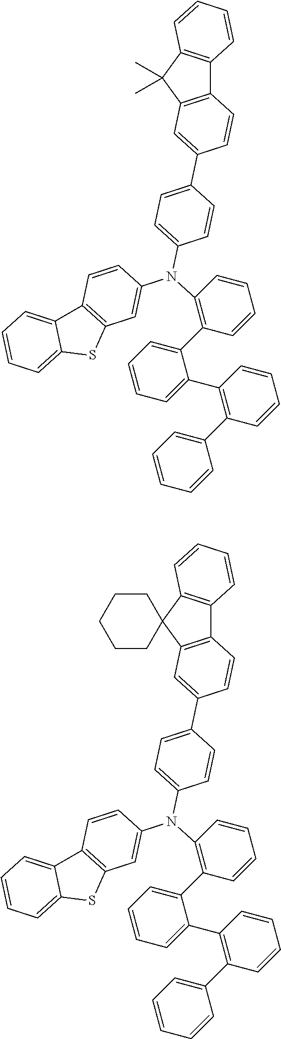 [SMILES: CC1(C)c2ccccc2-c2ccc(-c3ccc(N(c4ccc5c(c4)sc4ccccc45)c4ccccc4-c4ccccc4-c4ccccc4-c4ccccc4)cc3)cc21 ; c1ccc(-c2ccccc2-c2ccccc2-c2ccccc2N(c2ccc(-c3ccc4c(c3)C3(CCCCC3)c3ccccc3-4)cc2)c2ccc3c(c2)sc2ccccc23)cc1]

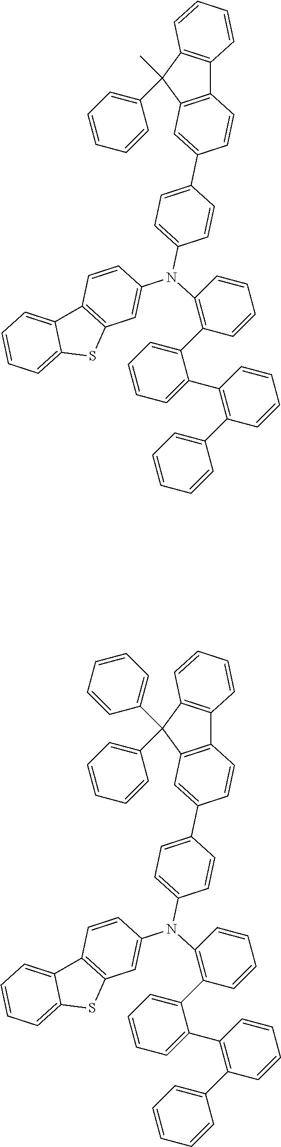 [SMILES: CC1(c2ccccc2)c2ccccc2-c2ccc(-c3ccc(N(c4ccc5c(c4)sc4ccccc45)c4ccccc4-c4ccccc4-c4ccccc4-c4ccccc4)cc3)cc21 ; c1ccc(-c2ccccc2-c2ccccc2-c2ccccc2N(c2ccc(-c3ccc4c(c3)C(c3ccccc3)(c3ccccc3)c3ccccc3-4)cc2)c2ccc3c(c2)sc2ccccc23)cc1]

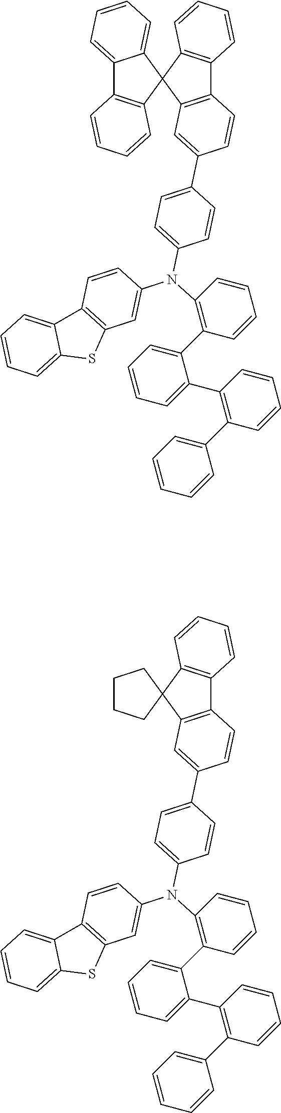 [SMILES: c1ccc(-c2ccccc2-c2ccccc2-c2ccccc2N(c2ccc(-c3ccc4c(c3)C3(c5ccccc5-c5ccccc53)c3ccccc3-4)cc2)c2ccc3c(c2)sc2ccccc23)cc1 ; c1ccc(-c2ccccc2-c2ccccc2-c2ccccc2N(c2ccc(-c3ccc4c(c3)C3(CCCC3)c3ccccc3-4)cc2)c2ccc3c(c2)sc2ccccc23)cc1]

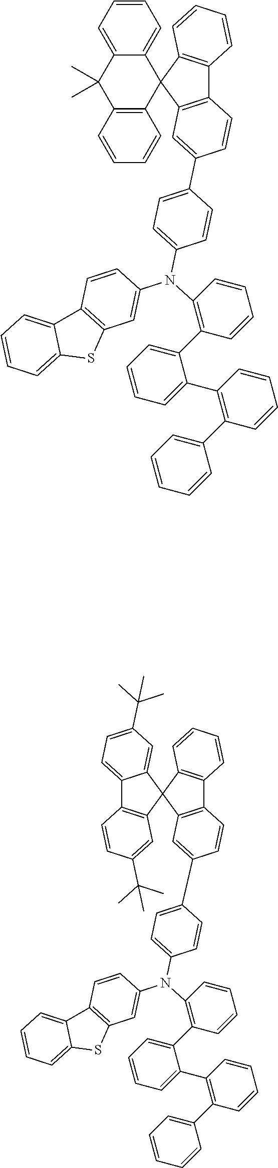 [SMILES: CC1(C)c2ccccc2C2(c3ccccc3-c3ccc(-c4ccc(N(c5ccc6c(c5)sc5ccccc56)c5ccccc5-c5ccccc5-c5ccccc5-c5ccccc5)cc4)cc32)c2ccccc21 ; CC(C)(C)c1ccc2c(c1)C1(c3ccccc3-c3ccc(-c4ccc(N(c5ccc6c(c5)sc5ccccc56)c5ccccc5-c5ccccc5-c5ccccc5-c5ccccc5)cc4)cc31)c1cc(C(C)(C)C)ccc1-2]

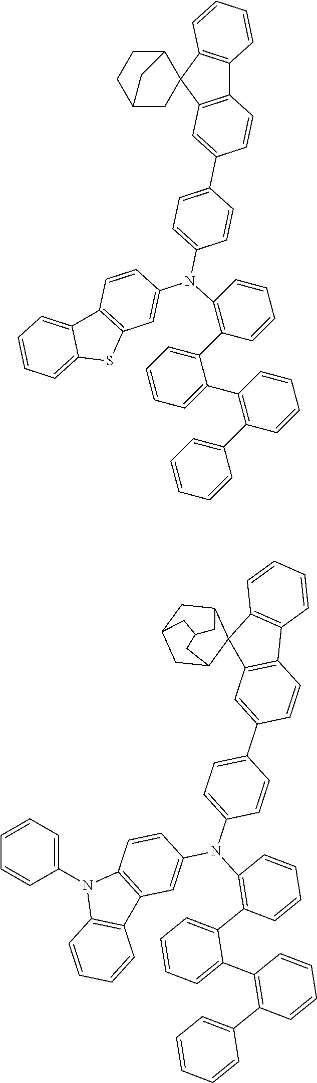 [SMILES: c1ccc(-c2ccccc2-c2ccccc2-c2ccccc2N(c2ccc(-c3ccc4c(c3)C3(CC5CCC3C5)c3ccccc3-4)cc2)c2ccc3c(c2)sc2ccccc23)cc1 ; c1ccc(-c2ccccc2-c2ccccc2-c2ccccc2N(c2ccc(-c3ccc4c(c3)C3(c5ccccc5-4)C4CC5CC(C4)CC3C5)cc2)c2ccc3c(c2)c2ccccc2n3-c2ccccc2)cc1]

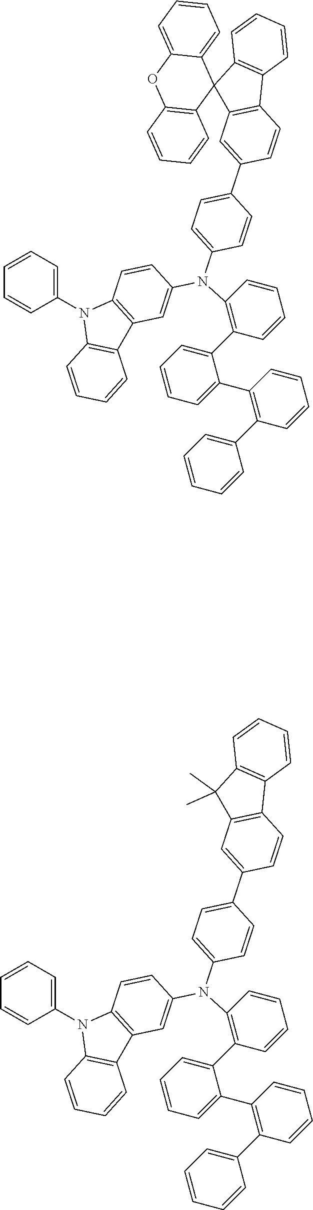 [SMILES: c1ccc(-c2ccccc2-c2ccccc2-c2ccccc2N(c2ccc(-c3ccc4c(c3)C3(c5ccccc5Oc5ccccc53)c3ccccc3-4)cc2)c2ccc3c(c2)c2ccccc2n3-c2ccccc2)cc1 ; CC1(C)c2ccccc2-c2ccc(-c3ccc(N(c4ccc5c(c4)c4ccccc4n5-c4ccccc4)c4ccccc4-c4ccccc4-c4ccccc4-c4ccccc4)cc3)cc21]

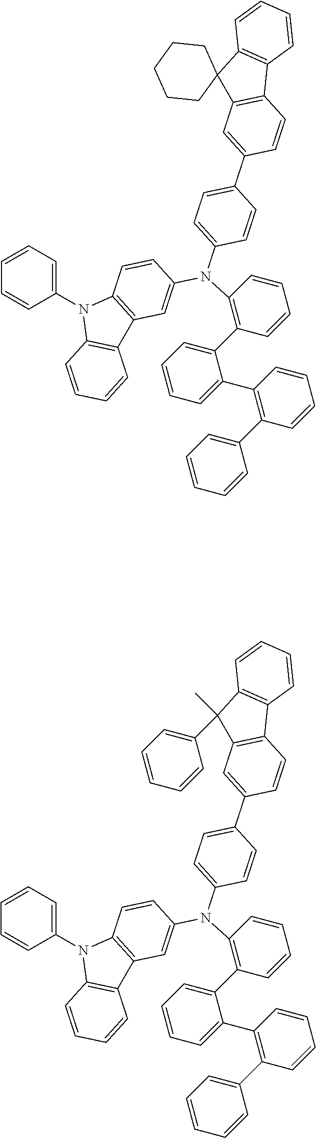 [SMILES: c1ccc(-c2ccccc2-c2ccccc2-c2ccccc2N(c2ccc(-c3ccc4c(c3)C3(CCCCC3)c3ccccc3-4)cc2)c2ccc3c(c2)c2ccccc2n3-c2ccccc2)cc1 ; CC1(c2ccccc2)c2ccccc2-c2ccc(-c3ccc(N(c4ccc5c(c4)c4ccccc4n5-c4ccccc4)c4ccccc4-c4ccccc4-c4ccccc4-c4ccccc4)cc3)cc21]

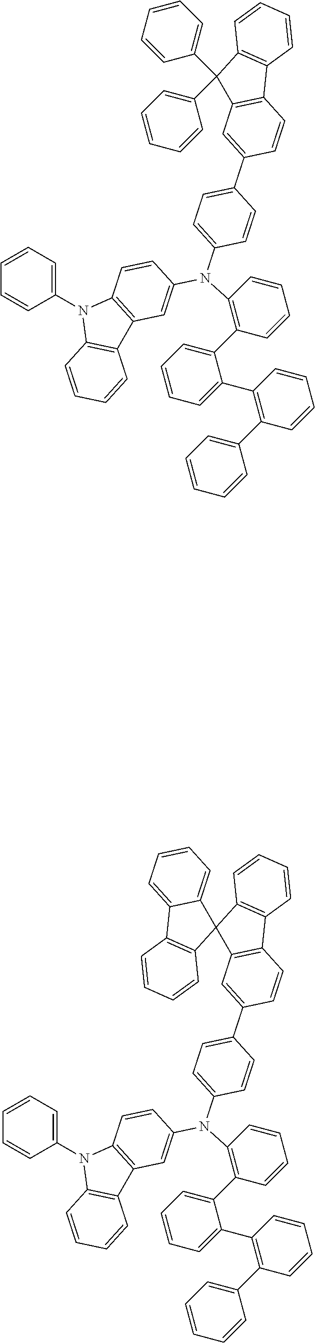 [SMILES: c1ccc(-c2ccccc2-c2ccccc2-c2ccccc2N(c2ccc(-c3ccc4c(c3)C(c3ccccc3)(c3ccccc3)c3ccccc3-4)cc2)c2ccc3c(c2)c2ccccc2n3-c2ccccc2)cc1 ; c1ccc(-c2ccccc2-c2ccccc2-c2ccccc2N(c2ccc(-c3ccc4c(c3)C3(c5ccccc5-c5ccccc53)c3ccccc3-4)cc2)c2ccc3c(c2)c2ccccc2n3-c2ccccc2)cc1]

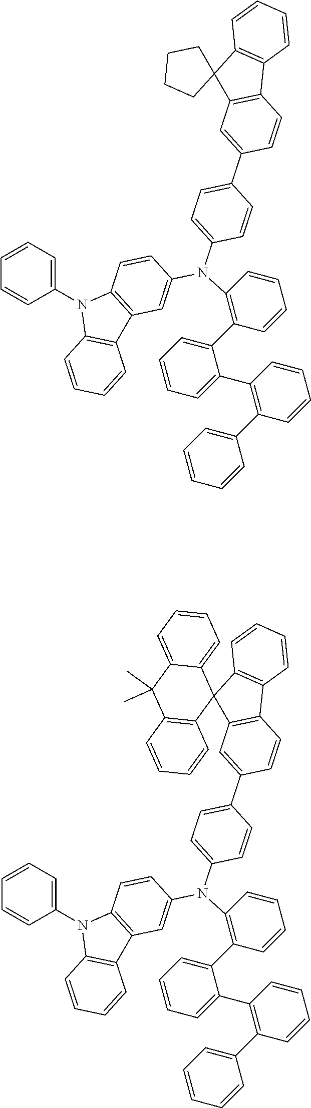 [SMILES: c1ccc(-c2ccccc2-c2ccccc2-c2ccccc2N(c2ccc(-c3ccc4c(c3)C3(CCCC3)c3ccccc3-4)cc2)c2ccc3c(c2)c2ccccc2n3-c2ccccc2)cc1 ; CC1(C)c2ccccc2C2(c3ccccc3-c3ccc(-c4ccc(N(c5ccc6c(c5)c5ccccc5n6-c5ccccc5)c5ccccc5-c5ccccc5-c5ccccc5-c5ccccc5)cc4)cc32)c2ccccc21]

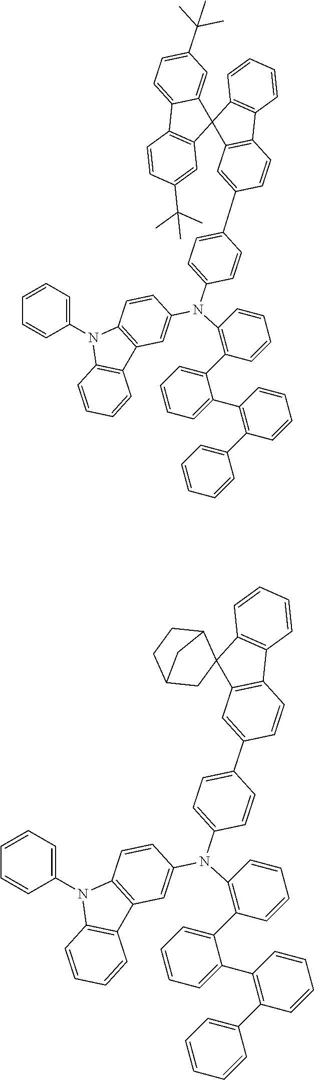 [SMILES: CC(C)(C)c1ccc2c(c1)C1(c3ccccc3-c3ccc(-c4ccc(N(c5ccc6c(c5)c5ccccc5n6-c5ccccc5)c5ccccc5-c5ccccc5-c5ccccc5-c5ccccc5)cc4)cc31)c1cc(C(C)(C)C)ccc1-2 ; c1ccc(-c2ccccc2-c2ccccc2-c2ccccc2N(c2ccc(-c3ccc4c(c3)C3(CC5CCC3C5)c3ccccc3-4)cc2)c2ccc3c(c2)c2ccccc2n3-c2ccccc2)cc1]

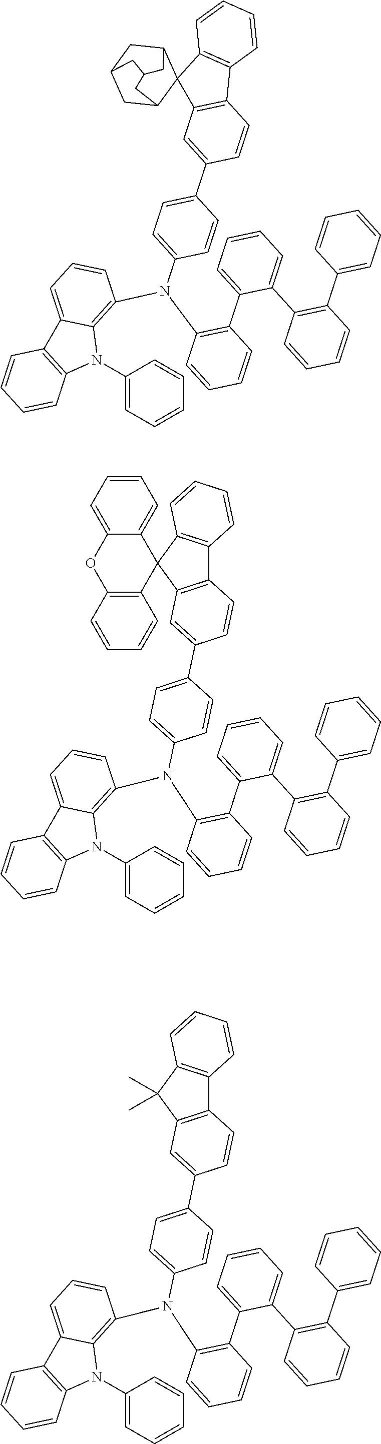 [SMILES: c1ccc(-c2ccccc2-c2ccccc2-c2ccccc2N(c2ccc(-c3ccc4c(c3)C3(c5ccccc5-4)C4CC5CC(C4)CC3C5)cc2)c2cccc3c4ccccc4n(-c4ccccc4)c23)cc1 ; c1ccc(-c2ccccc2-c2ccccc2-c2ccccc2N(c2ccc(-c3ccc4c(c3)C3(c5ccccc5Oc5ccccc53)c3ccccc3-4)cc2)c2cccc3c4ccccc4n(-c4ccccc4)c23)cc1 ; CC1(C)c2ccccc2-c2ccc(-c3ccc(N(c4ccccc4-c4ccccc4-c4ccccc4-c4ccccc4)c4cccc5c6ccccc6n(-c6ccccc6)c45)cc3)cc21]

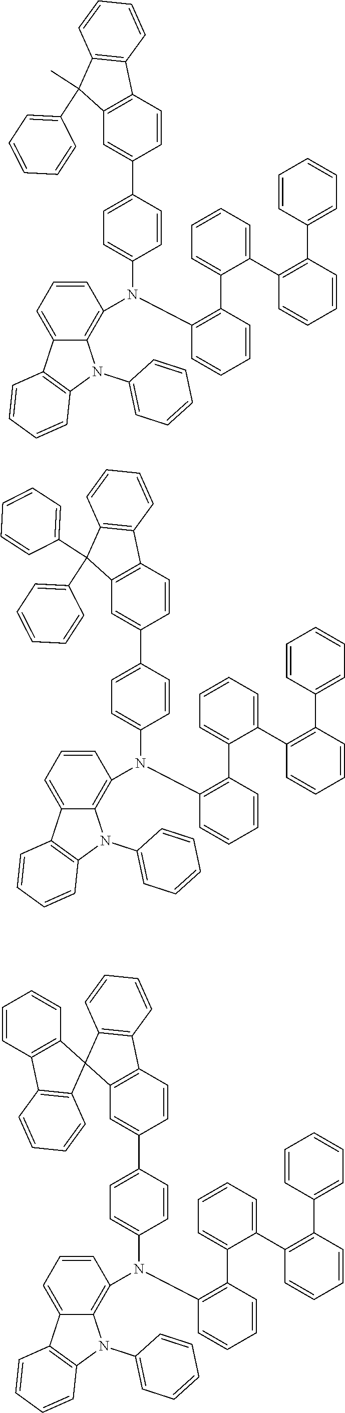 [SMILES: CC1(c2ccccc2)c2ccccc2-c2ccc(-c3ccc(N(c4ccccc4-c4ccccc4-c4ccccc4-c4ccccc4)c4cccc5c6ccccc6n(-c6ccccc6)c45)cc3)cc21 ; c1ccc(-c2ccccc2-c2ccccc2-c2ccccc2N(c2ccc(-c3ccc4c(c3)C(c3ccccc3)(c3ccccc3)c3ccccc3-4)cc2)c2cccc3c4ccccc4n(-c4ccccc4)c23)cc1 ; c1ccc(-c2ccccc2-c2ccccc2-c2ccccc2N(c2ccc(-c3ccc4c(c3)C3(c5ccccc5-c5ccccc53)c3ccccc3-4)cc2)c2cccc3c4ccccc4n(-c4ccccc4)c23)cc1]

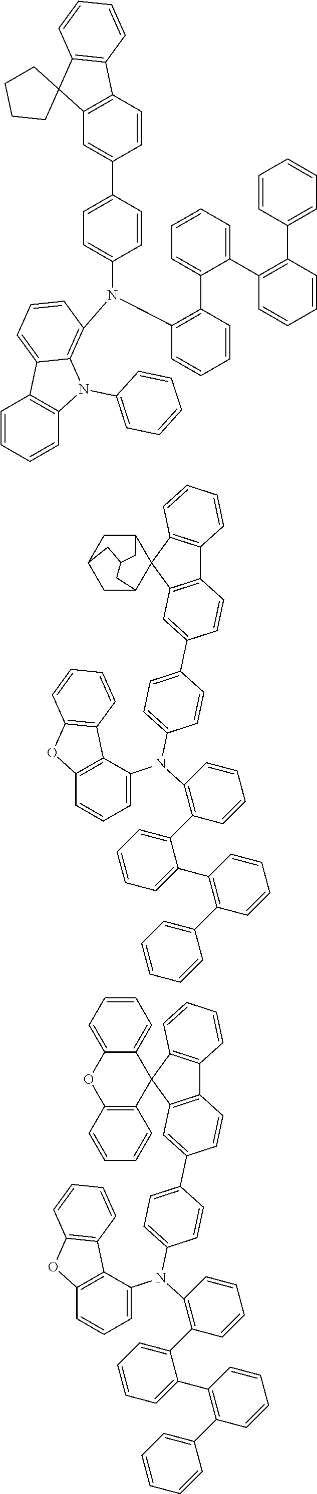

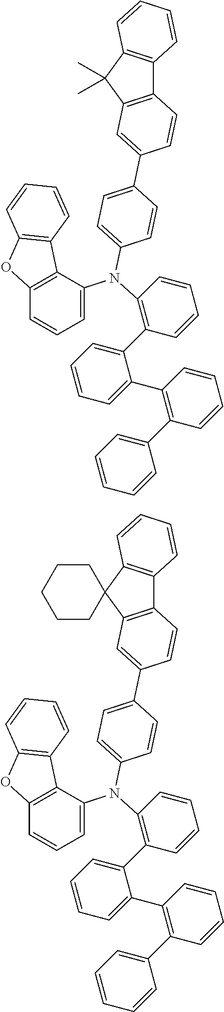

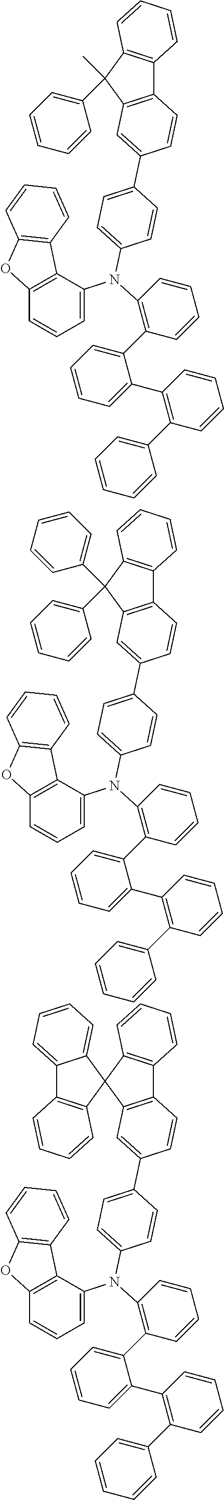

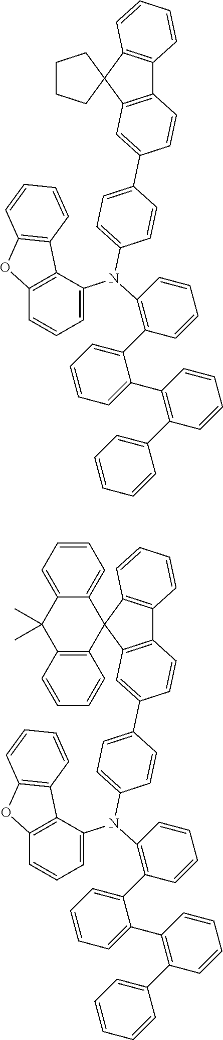 [SMILES: c1ccc(-c2ccccc2-c2ccccc2-c2ccccc2N(c2ccc(-c3ccc4c(c3)C3(CCCC3)c3ccccc3-4)cc2)c2cccc3oc4ccccc4c23)cc1 ; CC1(C)c2ccccc2C2(c3ccccc3-c3ccc(-c4ccc(N(c5ccccc5-c5ccccc5-c5ccccc5-c5ccccc5)c5cccc6oc7ccccc7c56)cc4)cc32)c2ccccc21]

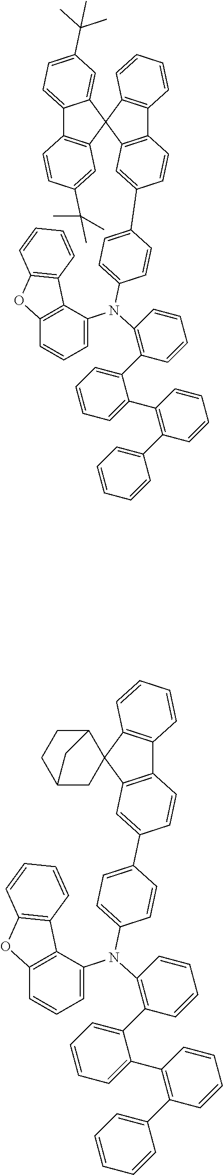 [SMILES: CC(C)(C)c1ccc2c(c1)C1(c3ccccc3-c3ccc(-c4ccc(N(c5ccccc5-c5ccccc5-c5ccccc5-c5ccccc5)c5cccc6oc7ccccc7c56)cc4)cc31)c1cc(C(C)(C)C)ccc1-2 ; c1ccc(-c2ccccc2-c2ccccc2-c2ccccc2N(c2ccc(-c3ccc4c(c3)C3(CC5CCC3C5)c3ccccc3-4)cc2)c2cccc3oc4ccccc4c23)cc1]

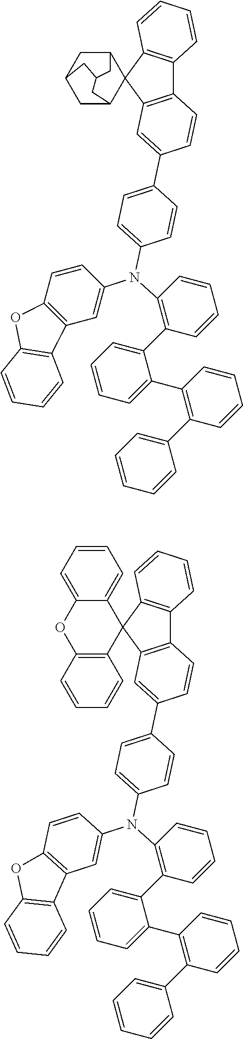 [SMILES: c1ccc(-c2ccccc2-c2ccccc2-c2ccccc2N(c2ccc(-c3ccc4c(c3)C3(c5ccccc5-4)C4CC5CC(C4)CC3C5)cc2)c2ccc3oc4ccccc4c3c2)cc1 ; c1ccc(-c2ccccc2-c2ccccc2-c2ccccc2N(c2ccc(-c3ccc4c(c3)C3(c5ccccc5Oc5ccccc53)c3ccccc3-4)cc2)c2ccc3oc4ccccc4c3c2)cc1]

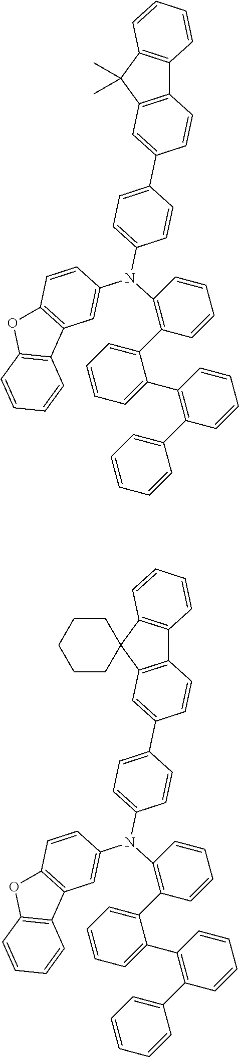 [SMILES: CC1(C)c2ccccc2-c2ccc(-c3ccc(N(c4ccc5oc6ccccc6c5c4)c4ccccc4-c4ccccc4-c4ccccc4-c4ccccc4)cc3)cc21 ; c1ccc(-c2ccccc2-c2ccccc2-c2ccccc2N(c2ccc(-c3ccc4c(c3)C3(CCCCC3)c3ccccc3-4)cc2)c2ccc3oc4ccccc4c3c2)cc1]

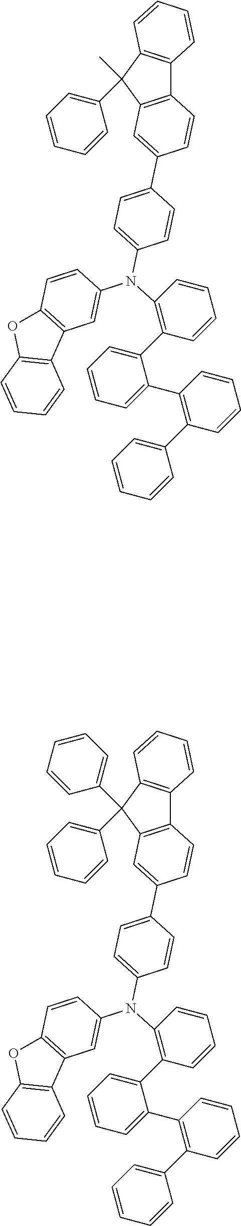 [SMILES: CC1(c2ccccc2)c2ccccc2-c2ccc(-c3ccc(N(c4ccc5oc6ccccc6c5c4)c4ccccc4-c4ccccc4-c4ccccc4-c4ccccc4)cc3)cc21 ; c1ccc(-c2ccccc2-c2ccccc2-c2ccccc2N(c2ccc(-c3ccc4c(c3)C(c3ccccc3)(c3ccccc3)c3ccccc3-4)cc2)c2ccc3oc4ccccc4c3c2)cc1]

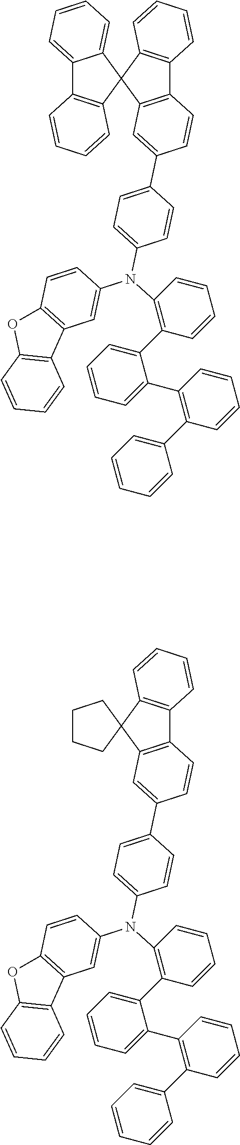 [SMILES: c1ccc(-c2ccccc2-c2ccccc2-c2ccccc2N(c2ccc(-c3ccc4c(c3)C3(c5ccccc5-c5ccccc53)c3ccccc3-4)cc2)c2ccc3oc4ccccc4c3c2)cc1 ; c1ccc(-c2ccccc2-c2ccccc2-c2ccccc2N(c2ccc(-c3ccc4c(c3)C3(CCCC3)c3ccccc3-4)cc2)c2ccc3oc4ccccc4c3c2)cc1]

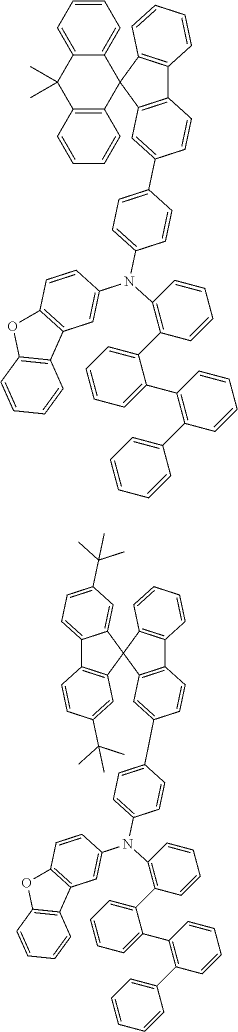 [SMILES: CC1(C)c2ccccc2C2(c3ccccc3-c3ccc(-c4ccc(N(c5ccc6oc7ccccc7c6c5)c5ccccc5-c5ccccc5-c5ccccc5-c5ccccc5)cc4)cc32)c2ccccc21 ; CC(C)(C)c1ccc2c(c1)C1(c3ccccc3-c3ccc(-c4ccc(N(c5ccc6oc7ccccc7c6c5)c5ccccc5-c5ccccc5-c5ccccc5-c5ccccc5)cc4)cc31)c1cc(C(C)(C)C)ccc1-2]

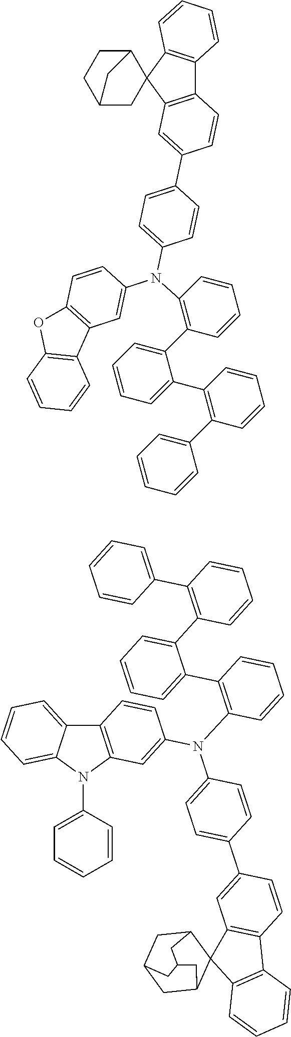 [SMILES: c1ccc(-c2ccccc2-c2ccccc2-c2ccccc2N(c2ccc(-c3ccc4c(c3)C3(CC5CCC3C5)c3ccccc3-4)cc2)c2ccc3oc4ccccc4c3c2)cc1 ; c1ccc(-c2ccccc2-c2ccccc2-c2ccccc2N(c2ccc(-c3ccc4c(c3)C3(c5ccccc5-4)C4CC5CC(C4)CC3C5)cc2)c2ccc3c4ccccc4n(-c4ccccc4)c3c2)cc1]

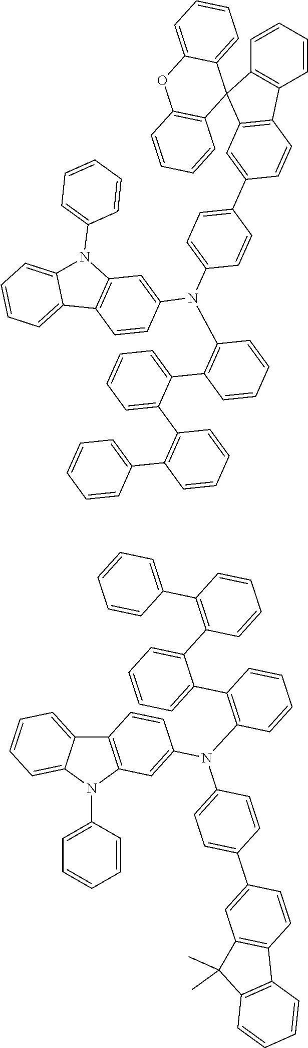 [SMILES: c1ccc(-c2ccccc2-c2ccccc2-c2ccccc2N(c2ccc(-c3ccc4c(c3)C3(c5ccccc5Oc5ccccc53)c3ccccc3-4)cc2)c2ccc3c4ccccc4n(-c4ccccc4)c3c2)cc1 ; CC1(C)c2ccccc2-c2ccc(-c3ccc(N(c4ccc5c6ccccc6n(-c6ccccc6)c5c4)c4ccccc4-c4ccccc4-c4ccccc4-c4ccccc4)cc3)cc21]

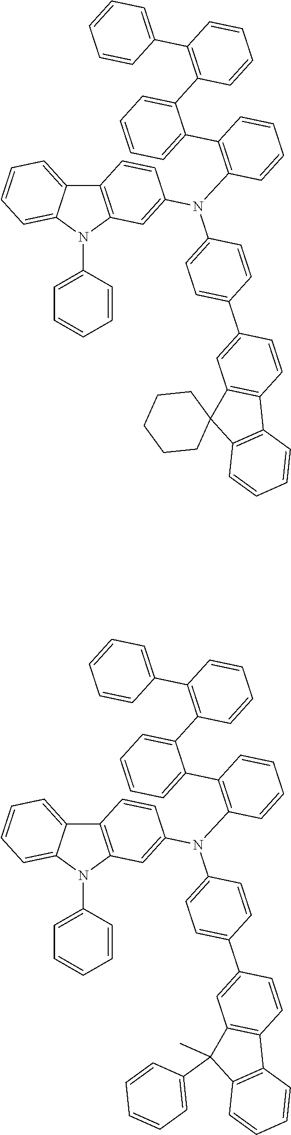 [SMILES: c1ccc(-c2ccccc2-c2ccccc2-c2ccccc2N(c2ccc(-c3ccc4c(c3)C3(CCCCC3)c3ccccc3-4)cc2)c2ccc3c4ccccc4n(-c4ccccc4)c3c2)cc1 ; CC1(c2ccccc2)c2ccccc2-c2ccc(-c3ccc(N(c4ccc5c6ccccc6n(-c6ccccc6)c5c4)c4ccccc4-c4ccccc4-c4ccccc4-c4ccccc4)cc3)cc21]

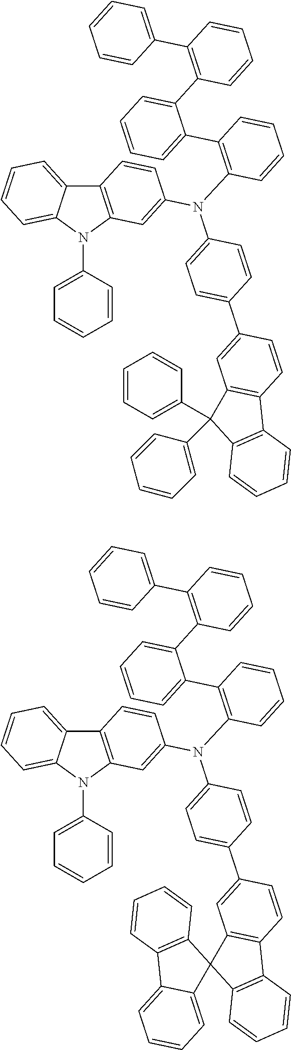 [SMILES: c1ccc(-c2ccccc2-c2ccccc2-c2ccccc2N(c2ccc(-c3ccc4c(c3)C(c3ccccc3)(c3ccccc3)c3ccccc3-4)cc2)c2ccc3c4ccccc4n(-c4ccccc4)c3c2)cc1 ; c1ccc(-c2ccccc2-c2ccccc2-c2ccccc2N(c2ccc(-c3ccc4c(c3)C3(c5ccccc5-c5ccccc53)c3ccccc3-4)cc2)c2ccc3c4ccccc4n(-c4ccccc4)c3c2)cc1]

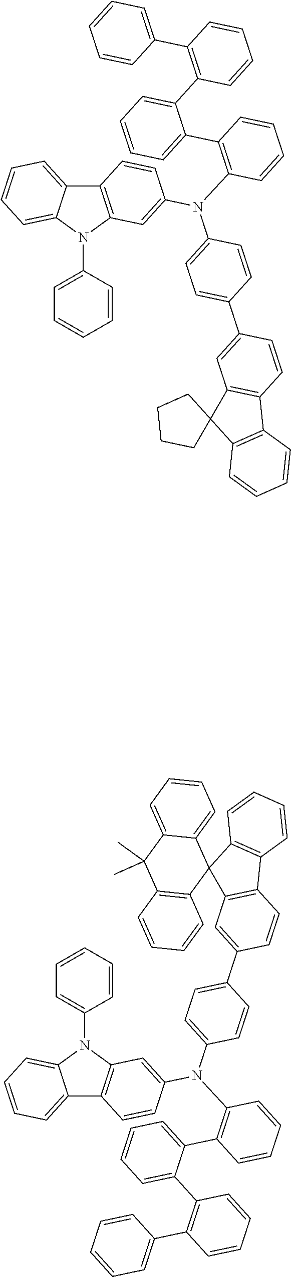 [SMILES: c1ccc(-c2ccccc2-c2ccccc2-c2ccccc2N(c2ccc(-c3ccc4c(c3)C3(CCCC3)c3ccccc3-4)cc2)c2ccc3c4ccccc4n(-c4ccccc4)c3c2)cc1 ; CC1(C)c2ccccc2C2(c3ccccc3-c3ccc(-c4ccc(N(c5ccc6c7ccccc7n(-c7ccccc7)c6c5)c5ccccc5-c5ccccc5-c5ccccc5-c5ccccc5)cc4)cc32)c2ccccc21]

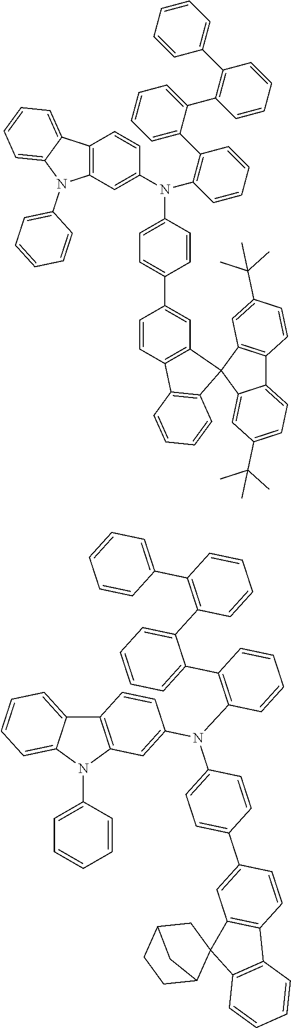 [SMILES: CC(C)(C)c1ccc2c(c1)C1(c3ccccc3-c3ccc(-c4ccc(N(c5ccc6c7ccccc7n(-c7ccccc7)c6c5)c5ccccc5-c5ccccc5-c5ccccc5-c5ccccc5)cc4)cc31)c1cc(C(C)(C)C)ccc1-2 ; c1ccc(-c2ccccc2-c2ccccc2-c2ccccc2N(c2ccc(-c3ccc4c(c3)C3(CC5CCC3C5)c3ccccc3-4)cc2)c2ccc3c4ccccc4n(-c4ccccc4)c3c2)cc1]

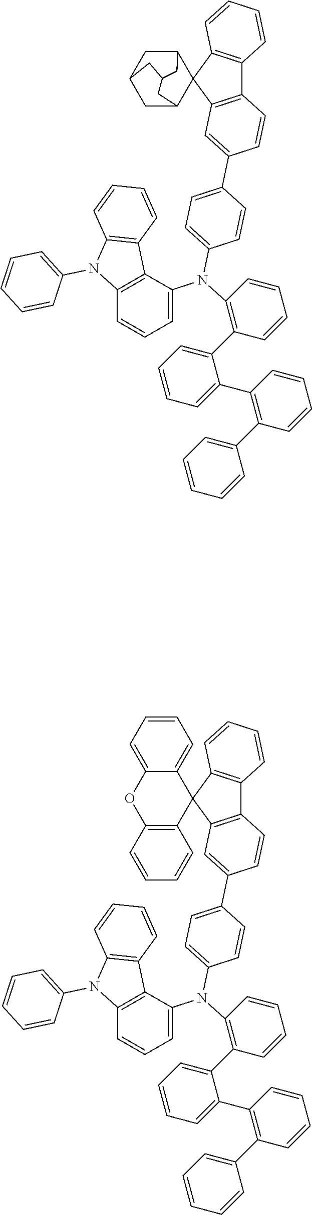 [SMILES: c1ccc(-c2ccccc2-c2ccccc2-c2ccccc2N(c2ccc(-c3ccc4c(c3)C3(c5ccccc5-4)C4CC5CC(C4)CC3C5)cc2)c2cccc3c2c2ccccc2n3-c2ccccc2)cc1 ; c1ccc(-c2ccccc2-c2ccccc2-c2ccccc2N(c2ccc(-c3ccc4c(c3)C3(c5ccccc5Oc5ccccc53)c3ccccc3-4)cc2)c2cccc3c2c2ccccc2n3-c2ccccc2)cc1]

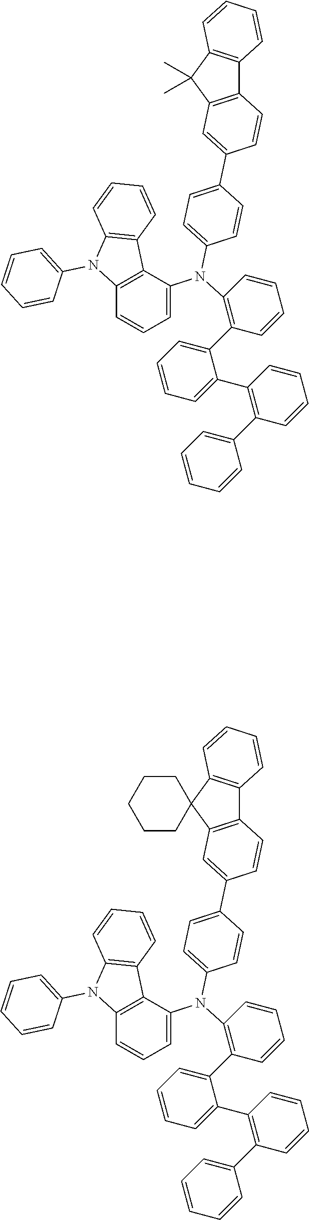 [SMILES: CC1(C)c2ccccc2-c2ccc(-c3ccc(N(c4ccccc4-c4ccccc4-c4ccccc4-c4ccccc4)c4cccc5c4c4ccccc4n5-c4ccccc4)cc3)cc21 ; c1ccc(-c2ccccc2-c2ccccc2-c2ccccc2N(c2ccc(-c3ccc4c(c3)C3(CCCCC3)c3ccccc3-4)cc2)c2cccc3c2c2ccccc2n3-c2ccccc2)cc1]

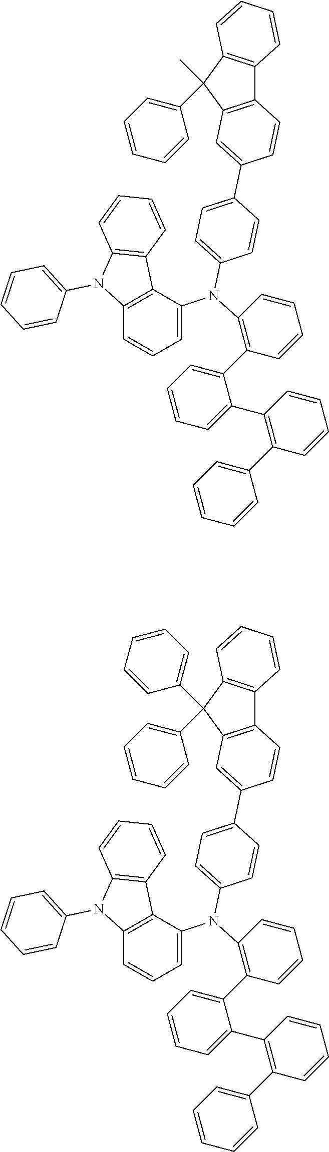 [SMILES: CC1(c2ccccc2)c2ccccc2-c2ccc(-c3ccc(N(c4ccccc4-c4ccccc4-c4ccccc4-c4ccccc4)c4cccc5c4c4ccccc4n5-c4ccccc4)cc3)cc21 ; c1ccc(-c2ccccc2-c2ccccc2-c2ccccc2N(c2ccc(-c3ccc4c(c3)C(c3ccccc3)(c3ccccc3)c3ccccc3-4)cc2)c2cccc3c2c2ccccc2n3-c2ccccc2)cc1]

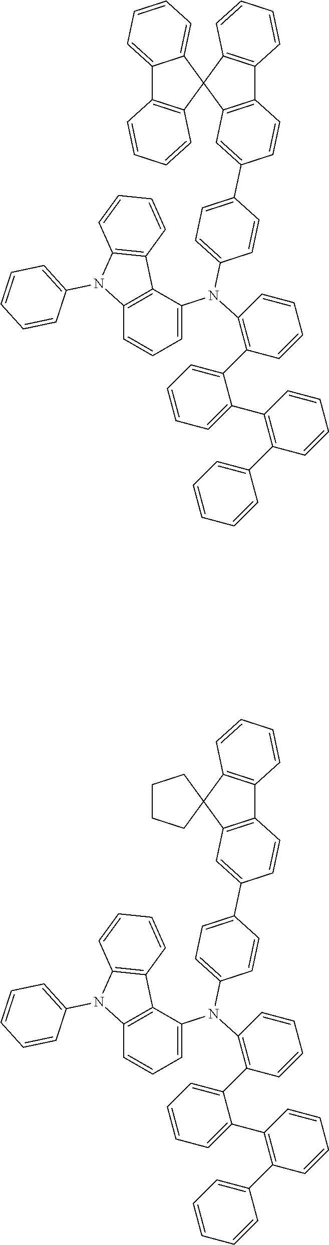 [SMILES: c1ccc(-c2ccccc2-c2ccccc2-c2ccccc2N(c2ccc(-c3ccc4c(c3)C3(c5ccccc5-c5ccccc53)c3ccccc3-4)cc2)c2cccc3c2c2ccccc2n3-c2ccccc2)cc1 ; c1ccc(-c2ccccc2-c2ccccc2-c2ccccc2N(c2ccc(-c3ccc4c(c3)C3(CCCC3)c3ccccc3-4)cc2)c2cccc3c2c2ccccc2n3-c2ccccc2)cc1]

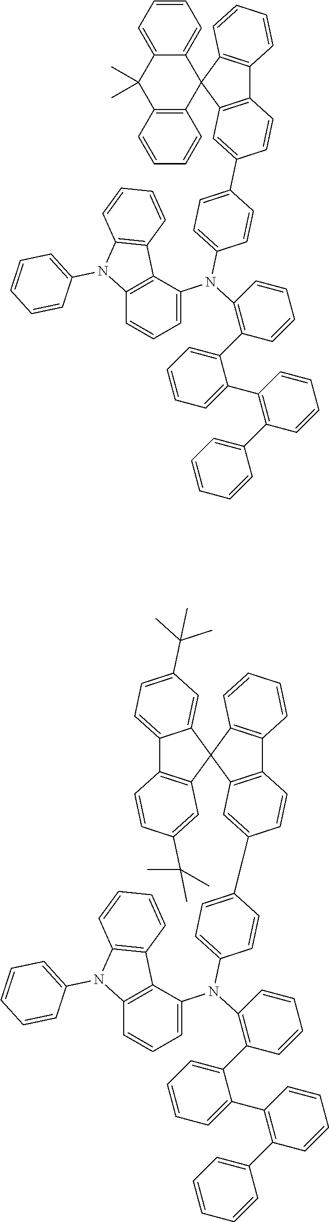 [SMILES: CC1(C)c2ccccc2C2(c3ccccc3-c3ccc(-c4ccc(N(c5ccccc5-c5ccccc5-c5ccccc5-c5ccccc5)c5cccc6c5c5ccccc5n6-c5ccccc5)cc4)cc32)c2ccccc21 ; CC(C)(C)c1ccc2c(c1)C1(c3ccccc3-c3ccc(-c4ccc(N(c5ccccc5-c5ccccc5-c5ccccc5-c5ccccc5)c5cccc6c5c5ccccc5n6-c5ccccc5)cc4)cc31)c1cc(C(C)(C)C)ccc1-2]

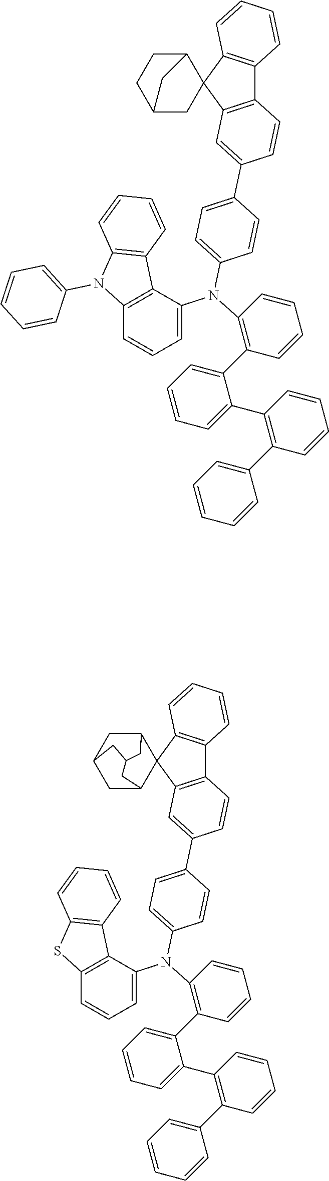 [SMILES: c1ccc(-c2ccccc2-c2ccccc2-c2ccccc2N(c2ccc(-c3ccc4c(c3)C3(CC5CCC3C5)c3ccccc3-4)cc2)c2cccc3c2c2ccccc2n3-c2ccccc2)cc1 ; c1ccc(-c2ccccc2-c2ccccc2-c2ccccc2N(c2ccc(-c3ccc4c(c3)C3(c5ccccc5-4)C4CC5CC(C4)CC3C5)cc2)c2cccc3sc4ccccc4c23)cc1]

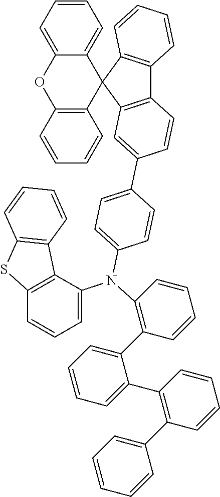 [SMILES: c1ccc(-c2ccccc2-c2ccccc2-c2ccccc2N(c2ccc(-c3ccc4c(c3)C3(c5ccccc5Oc5ccccc53)c3ccccc3-4)cc2)c2cccc3sc4ccccc4c23)cc1]

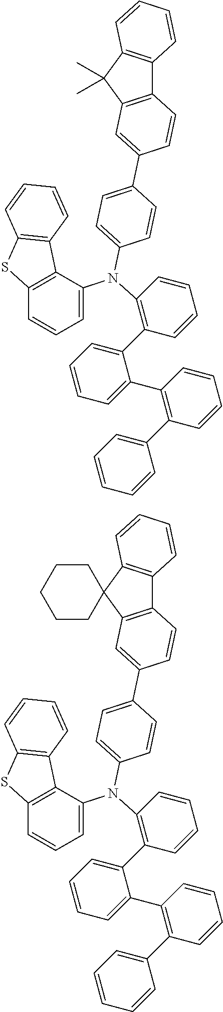

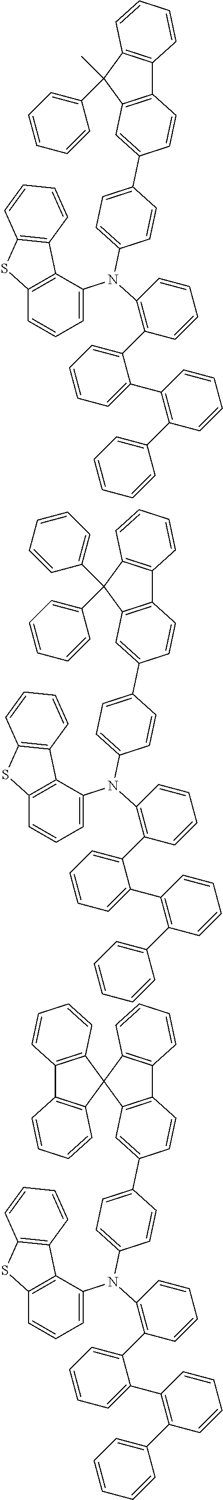

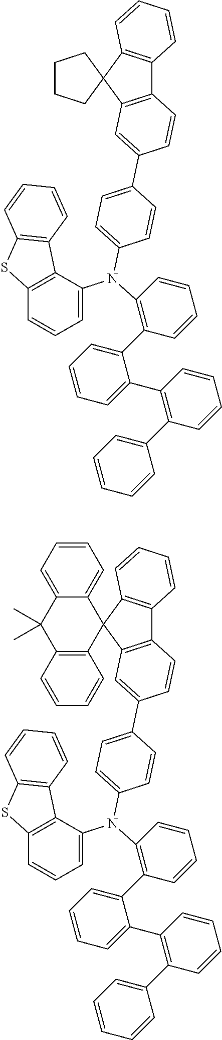 [SMILES: c1ccc(-c2ccccc2-c2ccccc2-c2ccccc2N(c2ccc(-c3ccc4c(c3)C3(CCCC3)c3ccccc3-4)cc2)c2cccc3sc4ccccc4c23)cc1 ; CC1(C)c2ccccc2C2(c3ccccc3-c3ccc(-c4ccc(N(c5ccccc5-c5ccccc5-c5ccccc5-c5ccccc5)c5cccc6sc7ccccc7c56)cc4)cc32)c2ccccc21]

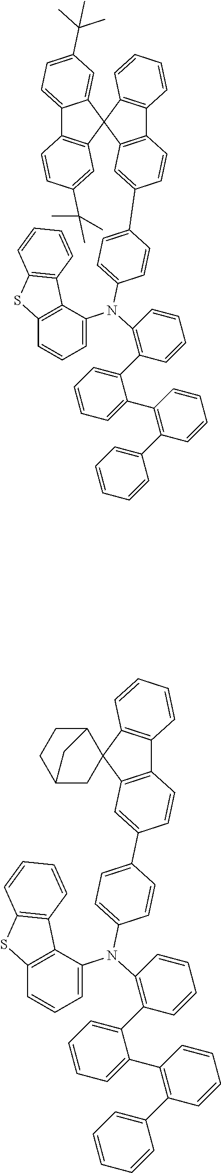 [SMILES: CC(C)(C)c1ccc2c(c1)C1(c3ccccc3-c3ccc(-c4ccc(N(c5ccccc5-c5ccccc5-c5ccccc5-c5ccccc5)c5cccc6sc7ccccc7c56)cc4)cc31)c1cc(C(C)(C)C)ccc1-2 ; c1ccc(-c2ccccc2-c2ccccc2-c2ccccc2N(c2ccc(-c3ccc4c(c3)C3(CC5CCC3C5)c3ccccc3-4)cc2)c2cccc3sc4ccccc4c23)cc1]

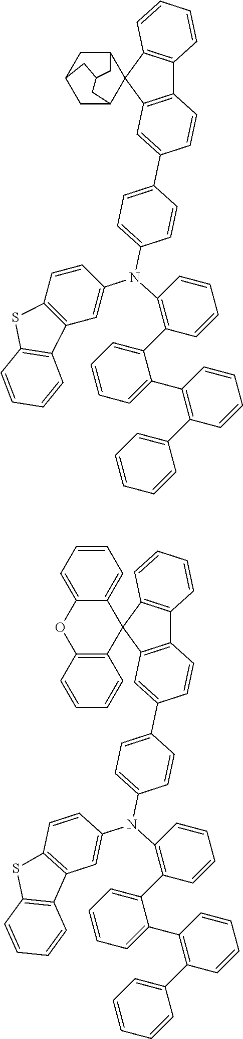 [SMILES: c1ccc(-c2ccccc2-c2ccccc2-c2ccccc2N(c2ccc(-c3ccc4c(c3)C3(c5ccccc5-4)C4CC5CC(C4)CC3C5)cc2)c2ccc3sc4ccccc4c3c2)cc1 ; c1ccc(-c2ccccc2-c2ccccc2-c2ccccc2N(c2ccc(-c3ccc4c(c3)C3(c5ccccc5Oc5ccccc53)c3ccccc3-4)cc2)c2ccc3sc4ccccc4c3c2)cc1]

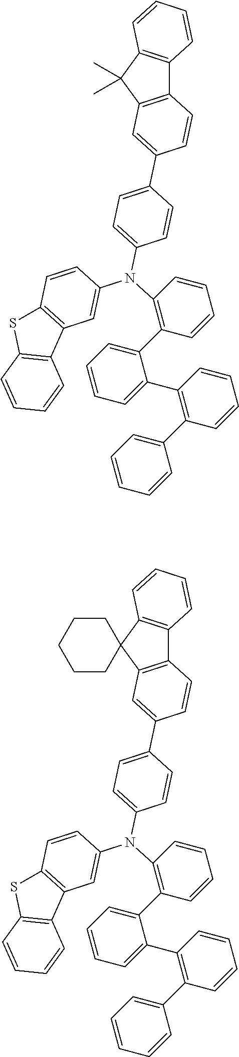 [SMILES: CC1(C)c2ccccc2-c2ccc(-c3ccc(N(c4ccc5sc6ccccc6c5c4)c4ccccc4-c4ccccc4-c4ccccc4-c4ccccc4)cc3)cc21 ; c1ccc(-c2ccccc2-c2ccccc2-c2ccccc2N(c2ccc(-c3ccc4c(c3)C3(CCCCC3)c3ccccc3-4)cc2)c2ccc3sc4ccccc4c3c2)cc1]

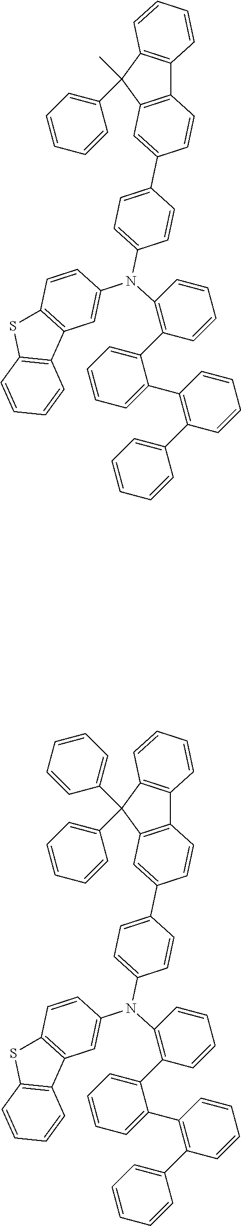 [SMILES: CC1(c2ccccc2)c2ccccc2-c2ccc(-c3ccc(N(c4ccc5sc6ccccc6c5c4)c4ccccc4-c4ccccc4-c4ccccc4-c4ccccc4)cc3)cc21 ; c1ccc(-c2ccccc2-c2ccccc2-c2ccccc2N(c2ccc(-c3ccc4c(c3)C(c3ccccc3)(c3ccccc3)c3ccccc3-4)cc2)c2ccc3sc4ccccc4c3c2)cc1]

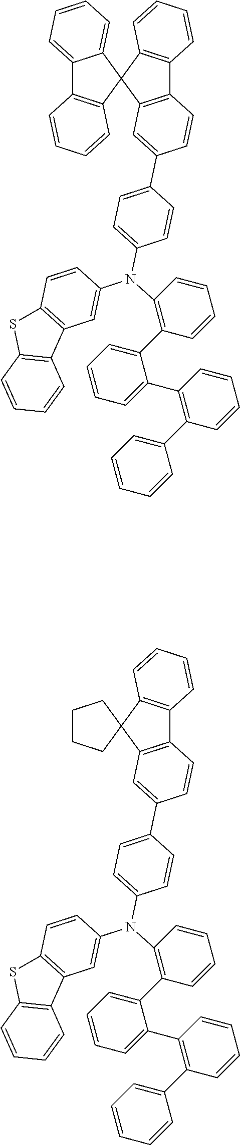 [SMILES: c1ccc(-c2ccccc2-c2ccccc2-c2ccccc2N(c2ccc(-c3ccc4c(c3)C3(c5ccccc5-c5ccccc53)c3ccccc3-4)cc2)c2ccc3sc4ccccc4c3c2)cc1 ; c1ccc(-c2ccccc2-c2ccccc2-c2ccccc2N(c2ccc(-c3ccc4c(c3)C3(CCCC3)c3ccccc3-4)cc2)c2ccc3sc4ccccc4c3c2)cc1]

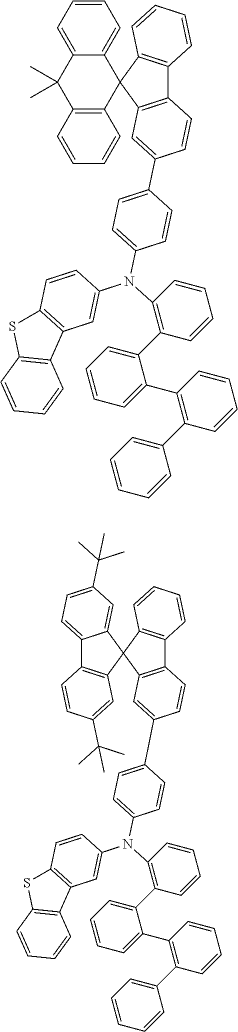 [SMILES: CC1(C)c2ccccc2C2(c3ccccc3-c3ccc(-c4ccc(N(c5ccc6sc7ccccc7c6c5)c5ccccc5-c5ccccc5-c5ccccc5-c5ccccc5)cc4)cc32)c2ccccc21 ; CC(C)(C)c1ccc2c(c1)C1(c3ccccc3-c3ccc(-c4ccc(N(c5ccc6sc7ccccc7c6c5)c5ccccc5-c5ccccc5-c5ccccc5-c5ccccc5)cc4)cc31)c1cc(C(C)(C)C)ccc1-2]

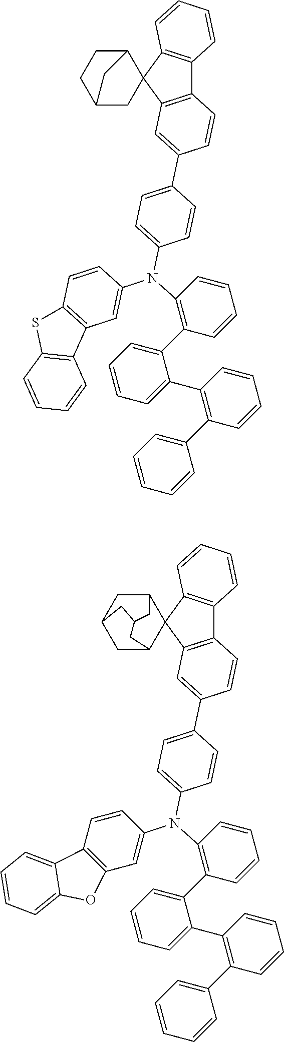 [SMILES: c1ccc(-c2ccccc2-c2ccccc2-c2ccccc2N(c2ccc(-c3ccc4c(c3)C3(CC5CCC3C5)c3ccccc3-4)cc2)c2ccc3sc4ccccc4c3c2)cc1 ; c1ccc(-c2ccccc2-c2ccccc2-c2ccccc2N(c2ccc(-c3ccc4c(c3)C3(c5ccccc5-4)C4CC5CC(C4)CC3C5)cc2)c2ccc3c(c2)oc2ccccc23)cc1]

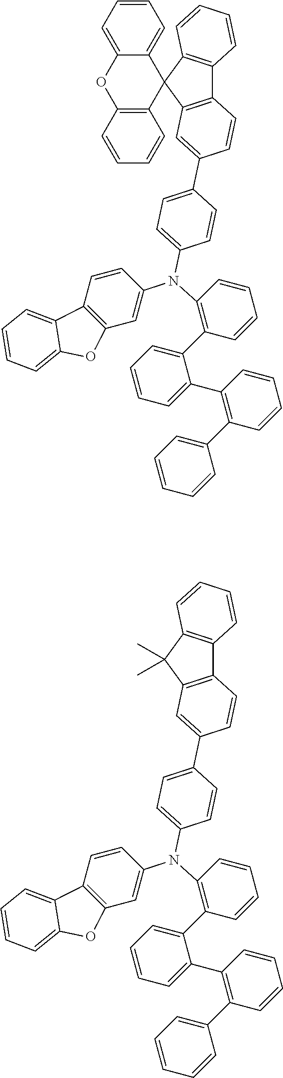 [SMILES: c1ccc(-c2ccccc2-c2ccccc2-c2ccccc2N(c2ccc(-c3ccc4c(c3)C3(c5ccccc5Oc5ccccc53)c3ccccc3-4)cc2)c2ccc3c(c2)oc2ccccc23)cc1 ; CC1(C)c2ccccc2-c2ccc(-c3ccc(N(c4ccc5c(c4)oc4ccccc45)c4ccccc4-c4ccccc4-c4ccccc4-c4ccccc4)cc3)cc21]

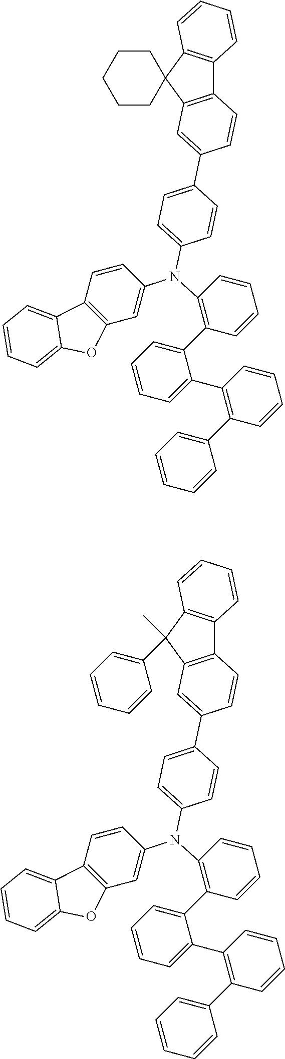 [SMILES: c1ccc(-c2ccccc2-c2ccccc2-c2ccccc2N(c2ccc(-c3ccc4c(c3)C3(CCCCC3)c3ccccc3-4)cc2)c2ccc3c(c2)oc2ccccc23)cc1 ; CC1(c2ccccc2)c2ccccc2-c2ccc(-c3ccc(N(c4ccc5c(c4)oc4ccccc45)c4ccccc4-c4ccccc4-c4ccccc4-c4ccccc4)cc3)cc21]

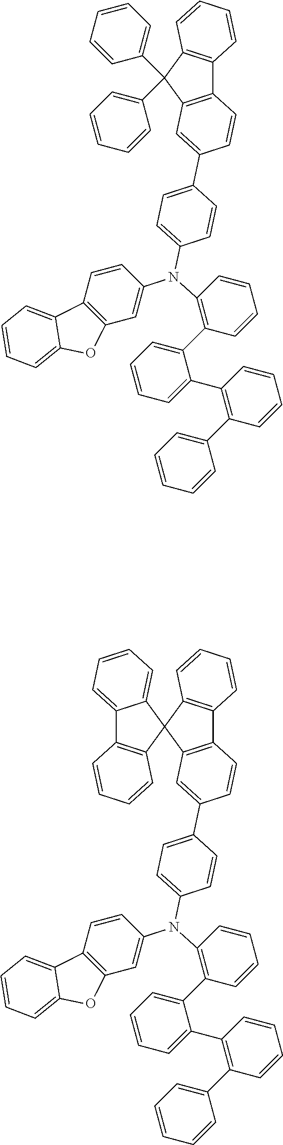 [SMILES: c1ccc(-c2ccccc2-c2ccccc2-c2ccccc2N(c2ccc(-c3ccc4c(c3)C(c3ccccc3)(c3ccccc3)c3ccccc3-4)cc2)c2ccc3c(c2)oc2ccccc23)cc1 ; c1ccc(-c2ccccc2-c2ccccc2-c2ccccc2N(c2ccc(-c3ccc4c(c3)C3(c5ccccc5-c5ccccc53)c3ccccc3-4)cc2)c2ccc3c(c2)oc2ccccc23)cc1]

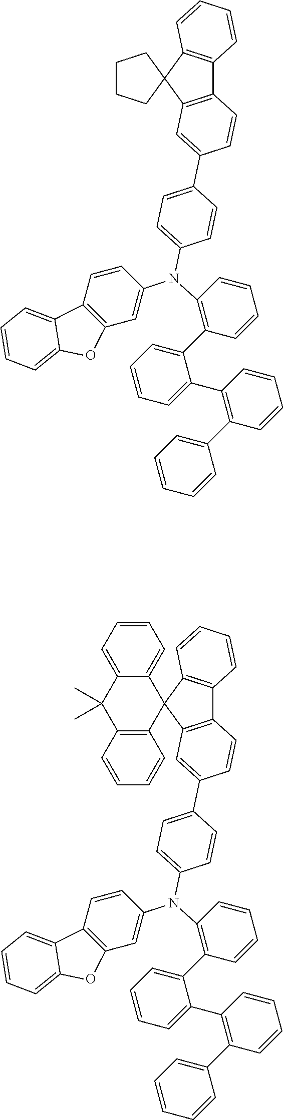 [SMILES: c1ccc(-c2ccccc2-c2ccccc2-c2ccccc2N(c2ccc(-c3ccc4c(c3)C3(CCCC3)c3ccccc3-4)cc2)c2ccc3c(c2)oc2ccccc23)cc1 ; CC1(C)c2ccccc2C2(c3ccccc3-c3ccc(-c4ccc(N(c5ccc6c(c5)oc5ccccc56)c5ccccc5-c5ccccc5-c5ccccc5-c5ccccc5)cc4)cc32)c2ccccc21]

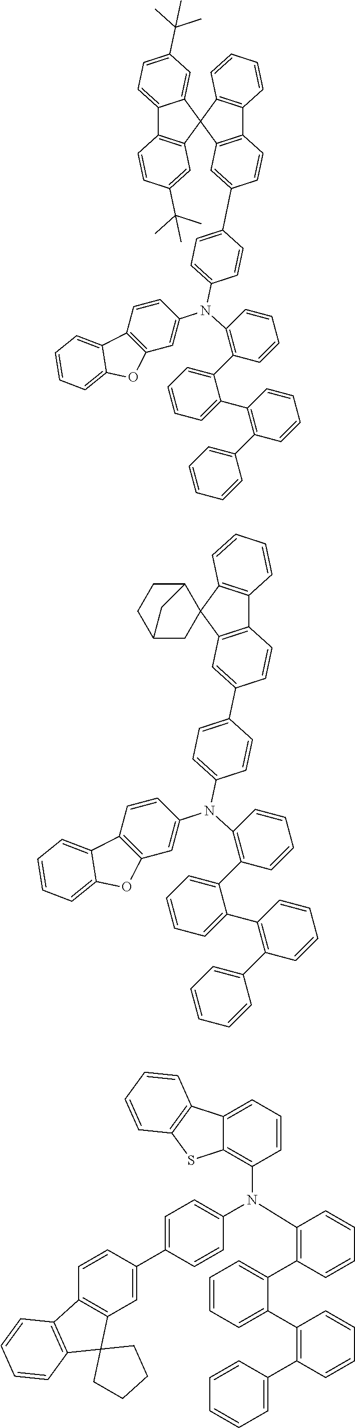

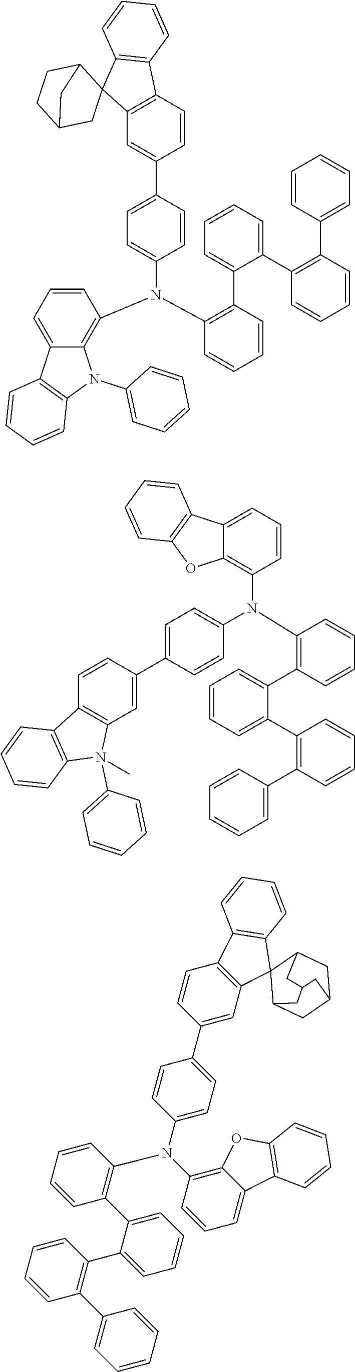

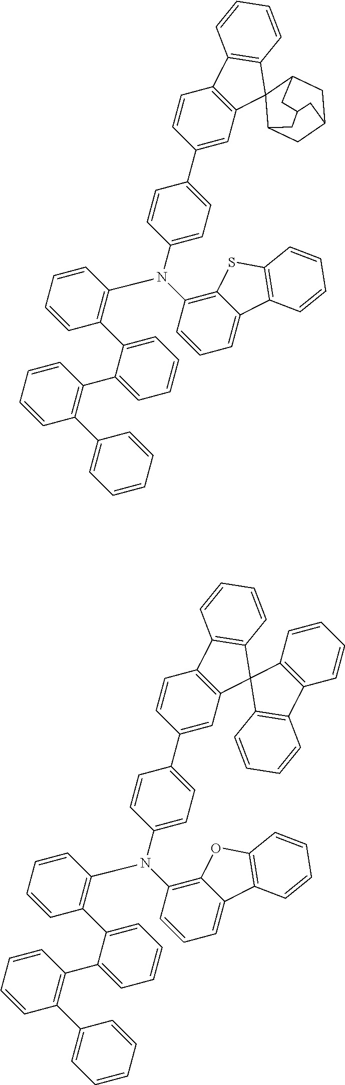 [SMILES: c1ccc(-c2ccccc2-c2ccccc2-c2ccccc2N(c2ccc(-c3ccc4c(c3)C3(c5ccccc5-4)C4CC5CC(C4)CC3C5)cc2)c2cccc3c2sc2ccccc23)cc1 ; c1ccc(-c2ccccc2-c2ccccc2-c2ccccc2N(c2ccc(-c3ccc4c(c3)C3(c5ccccc5-c5ccccc53)c3ccccc3-4)cc2)c2cccc3c2oc2ccccc23)cc1]

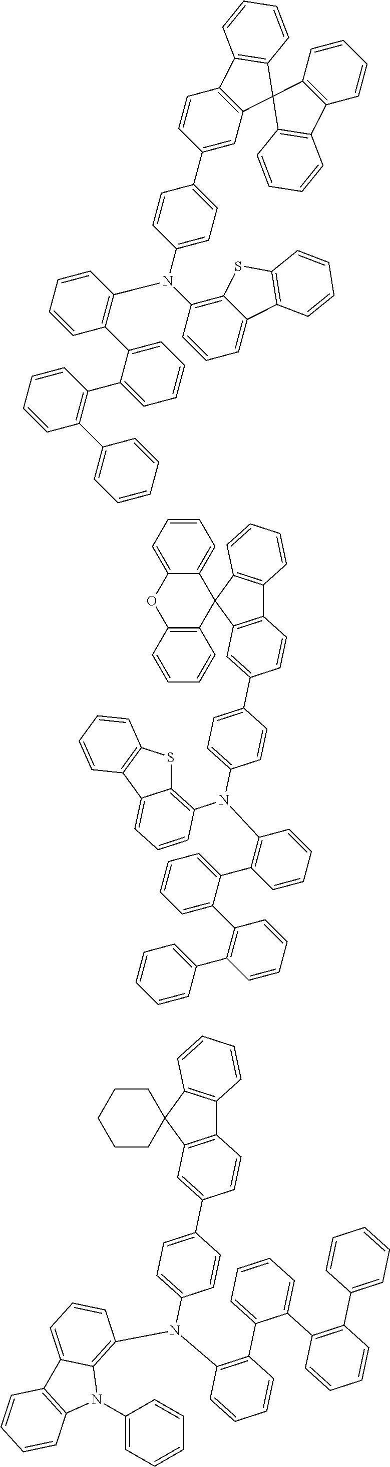

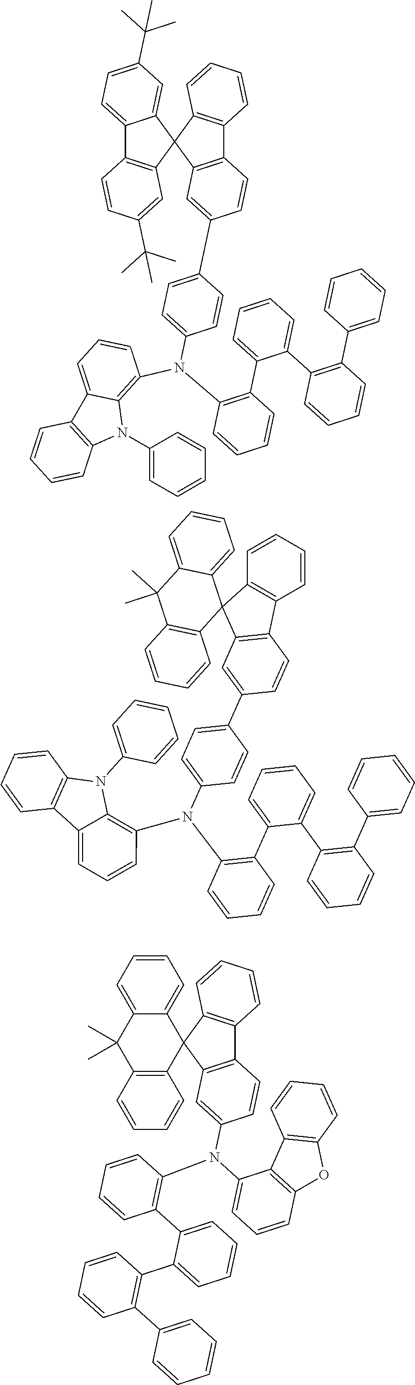

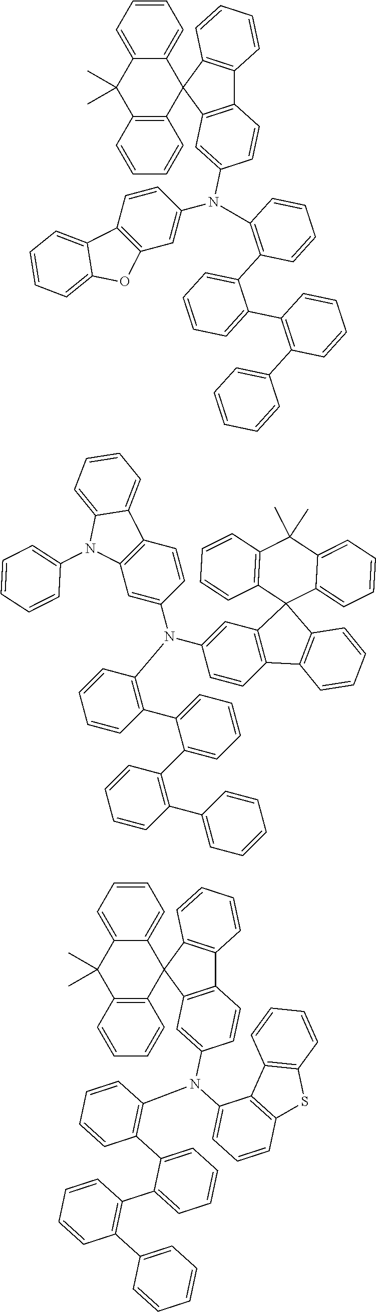

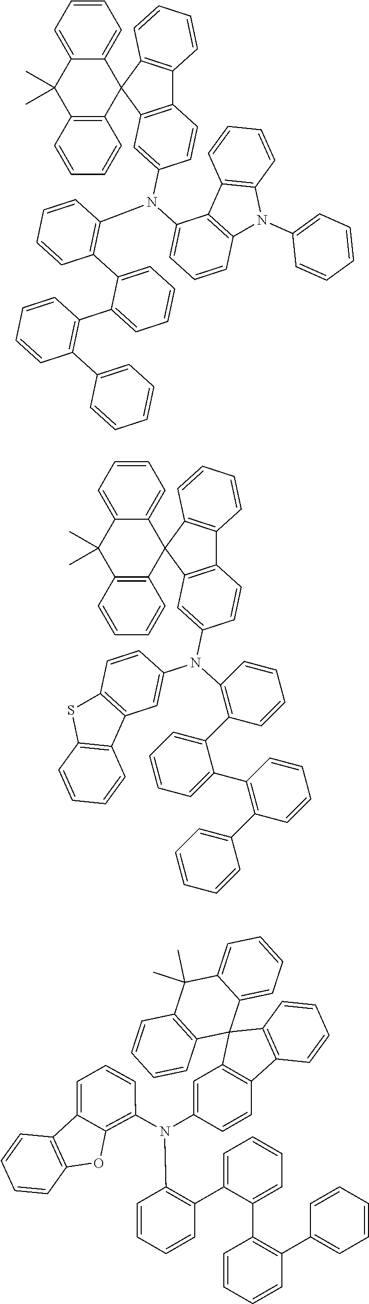

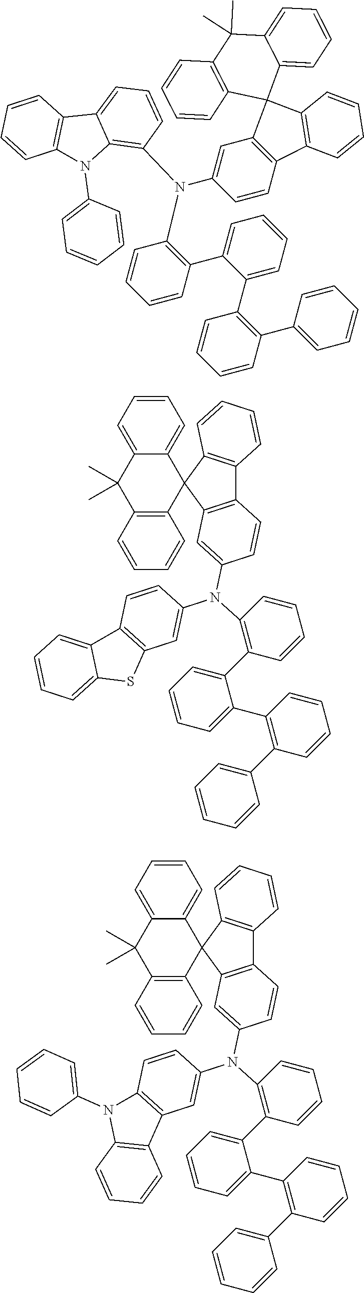

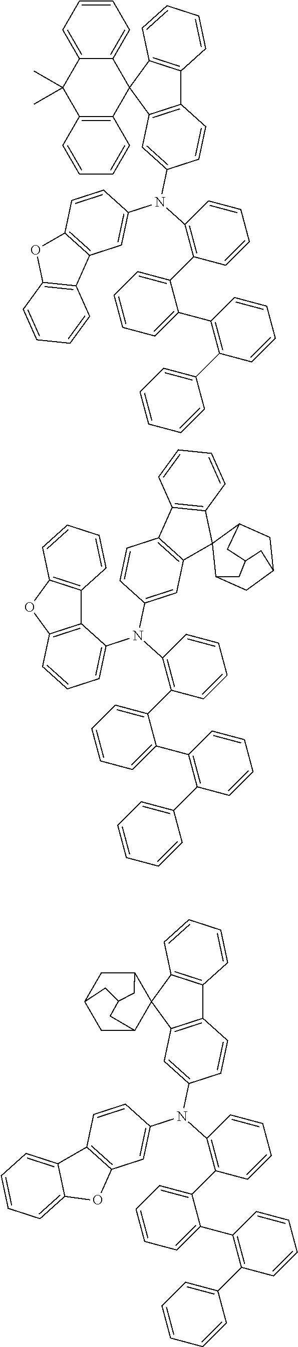

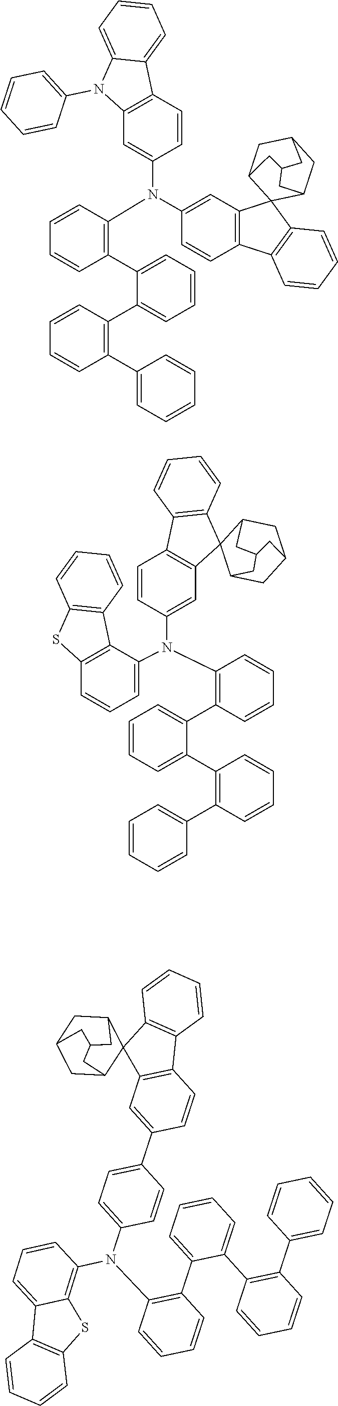

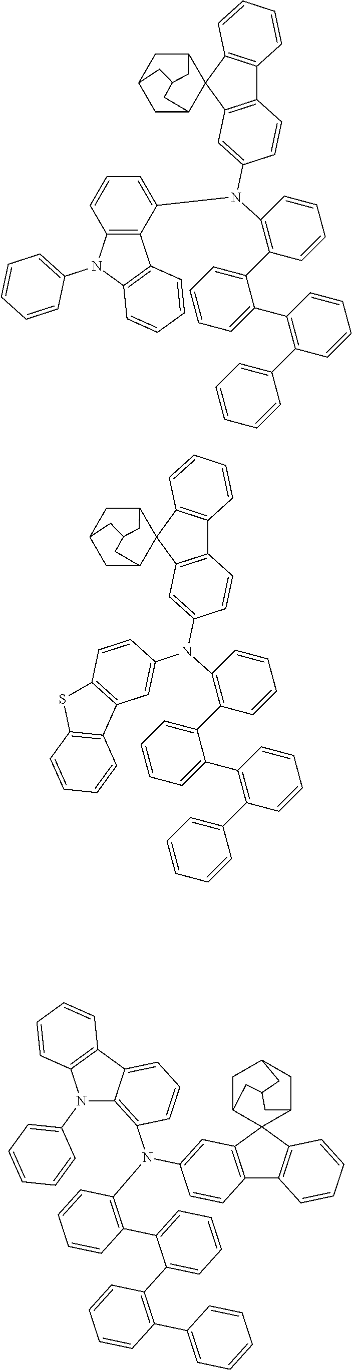

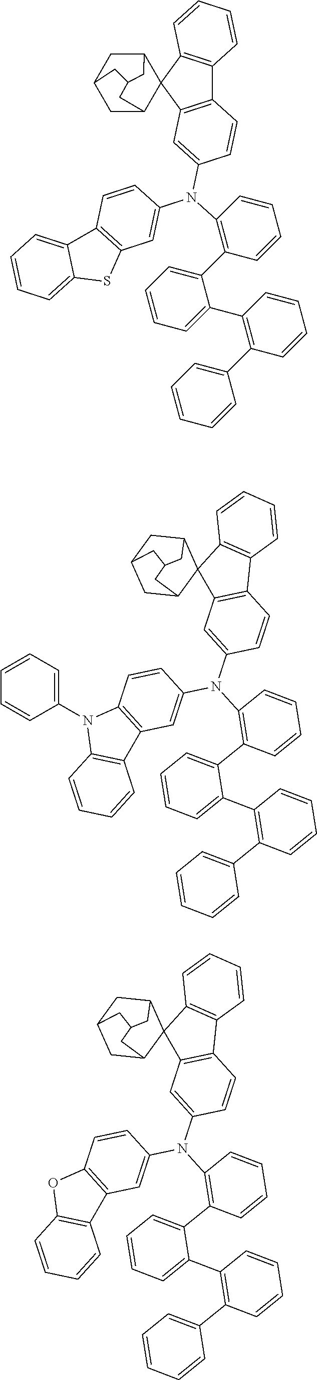

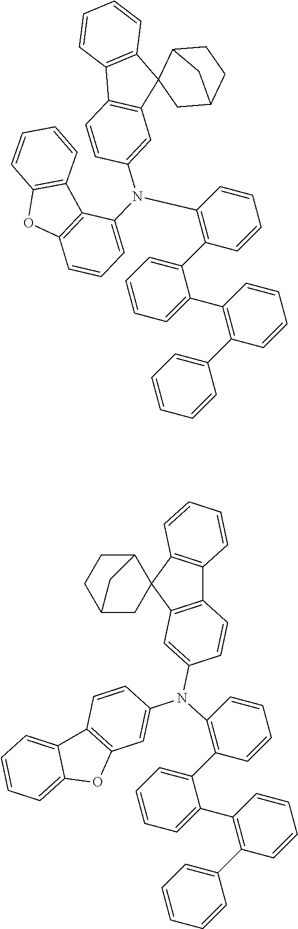 [SMILES: c1ccc(-c2ccccc2-c2ccccc2-c2ccccc2N(c2ccc3c(c2)C2(CC4CCC2C4)c2ccccc2-3)c2cccc3oc4ccccc4c23)cc1 ; c1ccc(-c2ccccc2-c2ccccc2-c2ccccc2N(c2ccc3c(c2)C2(CC4CCC2C4)c2ccccc2-3)c2ccc3c(c2)oc2ccccc23)cc1]

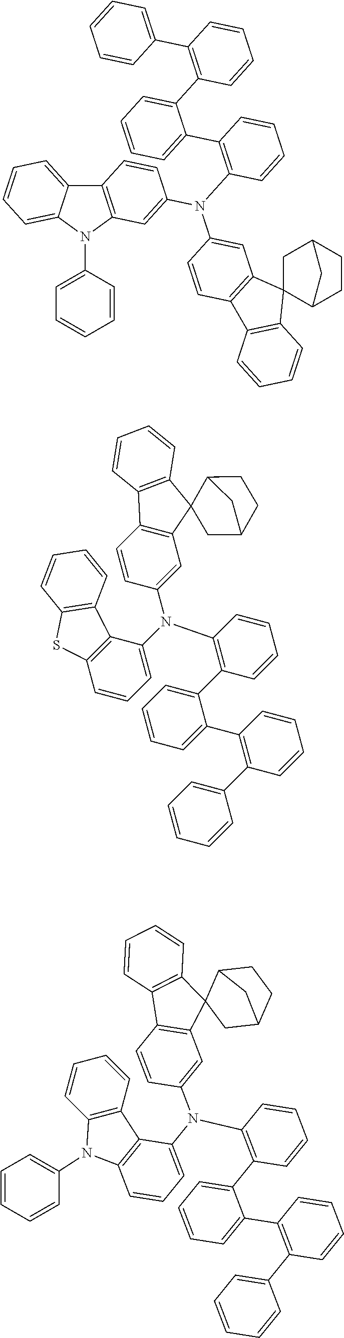 [SMILES: c1ccc(-c2ccccc2-c2ccccc2-c2ccccc2N(c2ccc3c(c2)C2(CC4CCC2C4)c2ccccc2-3)c2ccc3c4ccccc4n(-c4ccccc4)c3c2)cc1 ; c1ccc(-c2ccccc2-c2ccccc2-c2ccccc2N(c2ccc3c(c2)C2(CC4CCC2C4)c2ccccc2-3)c2cccc3sc4ccccc4c23)cc1 ; c1ccc(-c2ccccc2-c2ccccc2-c2ccccc2N(c2ccc3c(c2)C2(CC4CCC2C4)c2ccccc2-3)c2cccc3c2c2ccccc2n3-c2ccccc2)cc1]

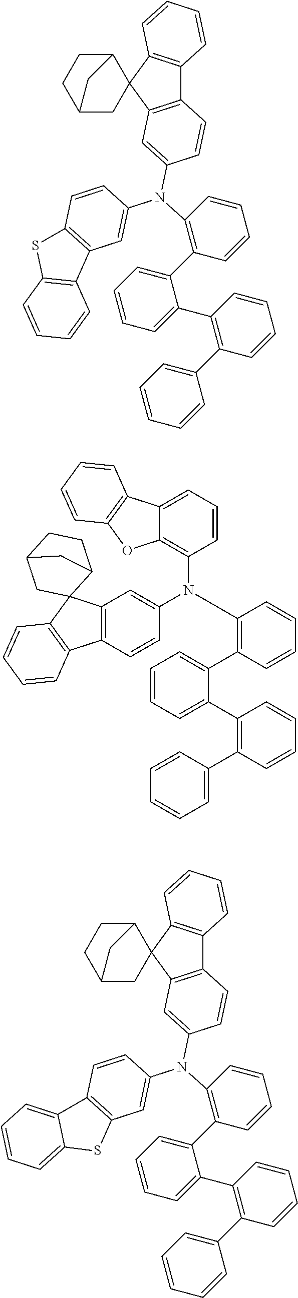 [SMILES: c1ccc(-c2ccccc2-c2ccccc2-c2ccccc2N(c2ccc3c(c2)C2(CC4CCC2C4)c2ccccc2-3)c2ccc3sc4ccccc4c3c2)cc1 ; c1ccc(-c2ccccc2-c2ccccc2-c2ccccc2N(c2ccc3c(c2)C2(CC4CCC2C4)c2ccccc2-3)c2cccc3c2oc2ccccc23)cc1 ; c1ccc(-c2ccccc2-c2ccccc2-c2ccccc2N(c2ccc3c(c2)C2(CC4CCC2C4)c2ccccc2-3)c2ccc3c(c2)sc2ccccc23)cc1]

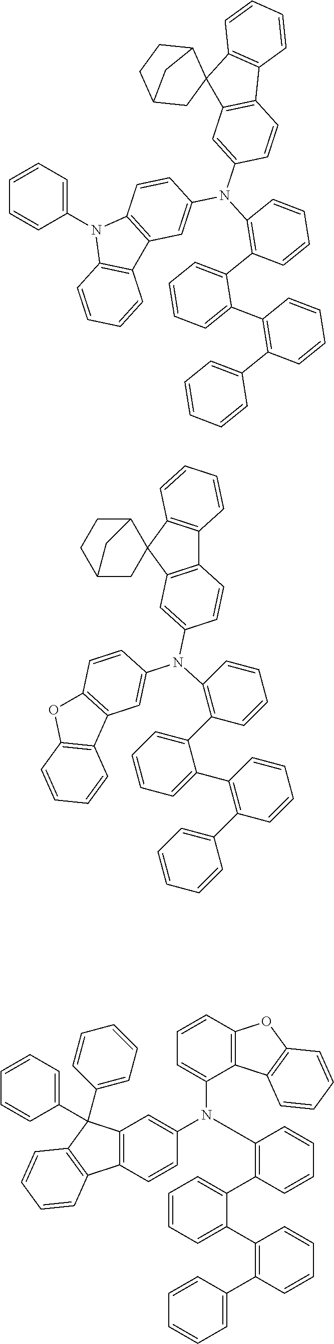 [SMILES: c1ccc(-c2ccccc2-c2ccccc2-c2ccccc2N(c2ccc3c(c2)C2(CC4CCC2C4)c2ccccc2-3)c2ccc3c(c2)c2ccccc2n3-c2ccccc2)cc1 ; c1ccc(-c2ccccc2-c2ccccc2-c2ccccc2N(c2ccc3c(c2)C2(CC4CCC2C4)c2ccccc2-3)c2ccc3oc4ccccc4c3c2)cc1 ; c1ccc(-c2ccccc2-c2ccccc2-c2ccccc2N(c2ccc3c(c2)C(c2ccccc2)(c2ccccc2)c2ccccc2-3)c2cccc3oc4ccccc4c23)cc1]

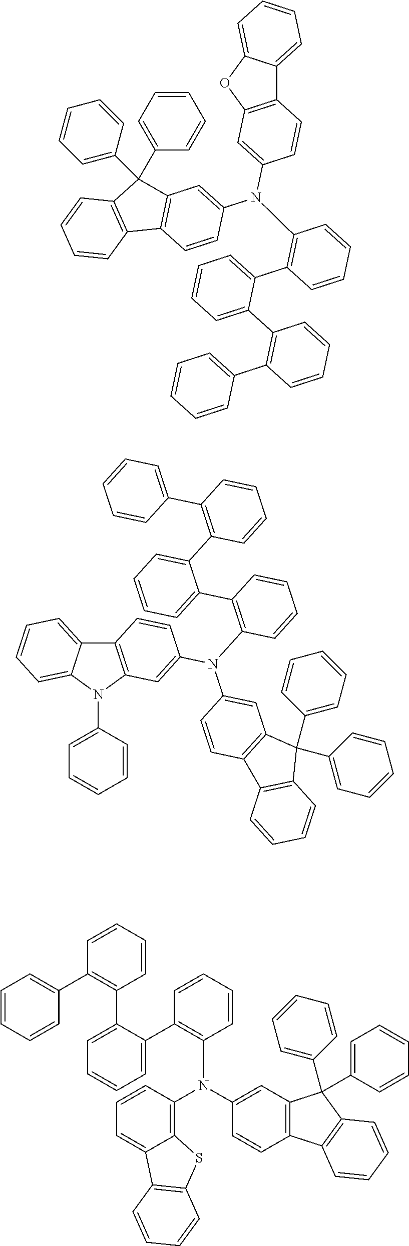 [SMILES: c1ccc(-c2ccccc2-c2ccccc2-c2ccccc2N(c2ccc3c(c2)C(c2ccccc2)(c2ccccc2)c2ccccc2-3)c2ccc3c(c2)oc2ccccc23)cc1 ; c1ccc(-c2ccccc2-c2ccccc2-c2ccccc2N(c2ccc3c(c2)C(c2ccccc2)(c2ccccc2)c2ccccc2-3)c2ccc3c4ccccc4n(-c4ccccc4)c3c2)cc1 ; c1ccc(-c2ccccc2-c2ccccc2-c2ccccc2N(c2ccc3c(c2)C(c2ccccc2)(c2ccccc2)c2ccccc2-3)c2cccc3c2sc2ccccc23)cc1]

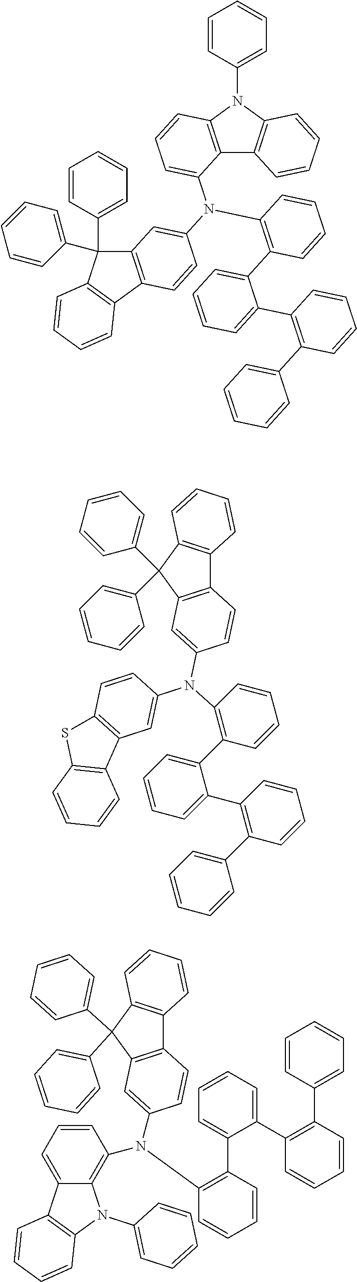 [SMILES: c1ccc(-c2ccccc2-c2ccccc2-c2ccccc2N(c2ccc3c(c2)C(c2ccccc2)(c2ccccc2)c2ccccc2-3)c2cccc3c2c2ccccc2n3-c2ccccc2)cc1 ; c1ccc(-c2ccccc2-c2ccccc2-c2ccccc2N(c2ccc3c(c2)C(c2ccccc2)(c2ccccc2)c2ccccc2-3)c2ccc3sc4ccccc4c3c2)cc1 ; c1ccc(-c2ccccc2-c2ccccc2-c2ccccc2N(c2ccc3c(c2)C(c2ccccc2)(c2ccccc2)c2ccccc2-3)c2cccc3c4ccccc4n(-c4ccccc4)c23)cc1]

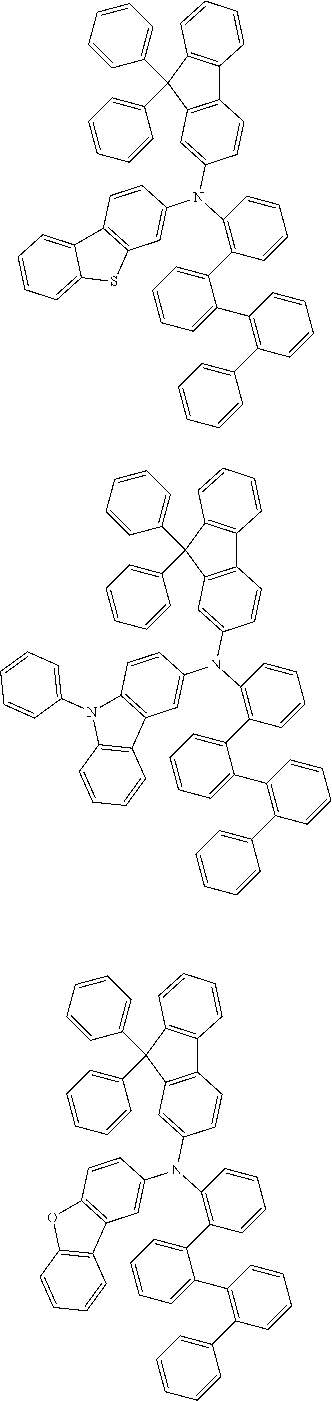

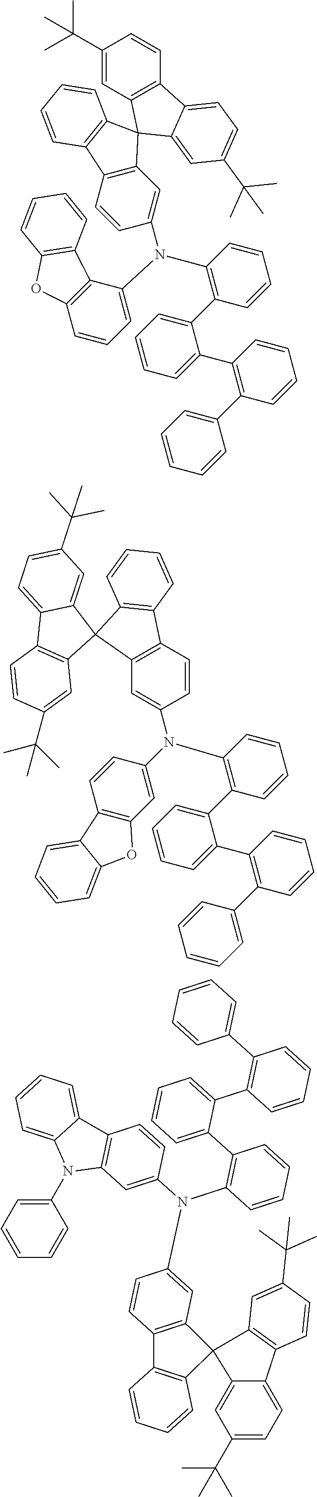

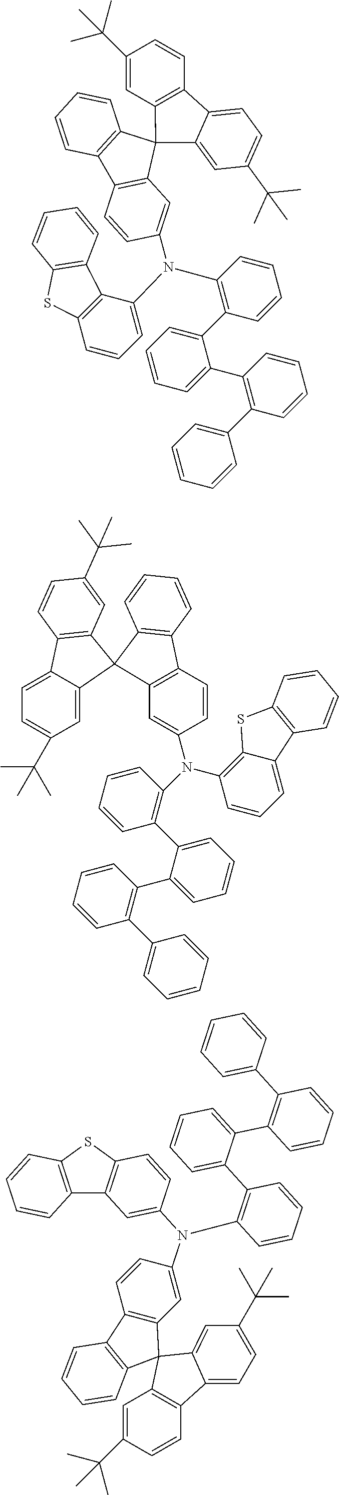

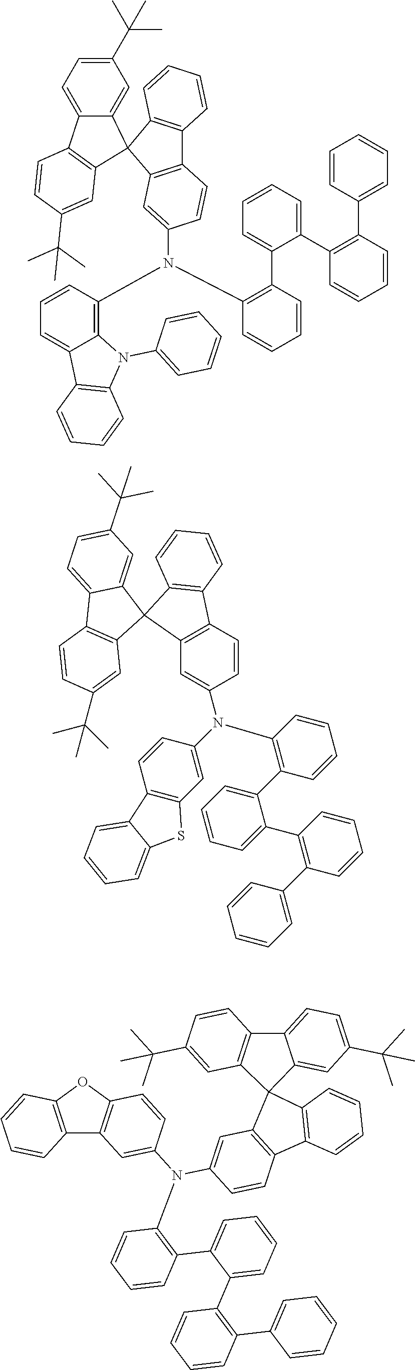

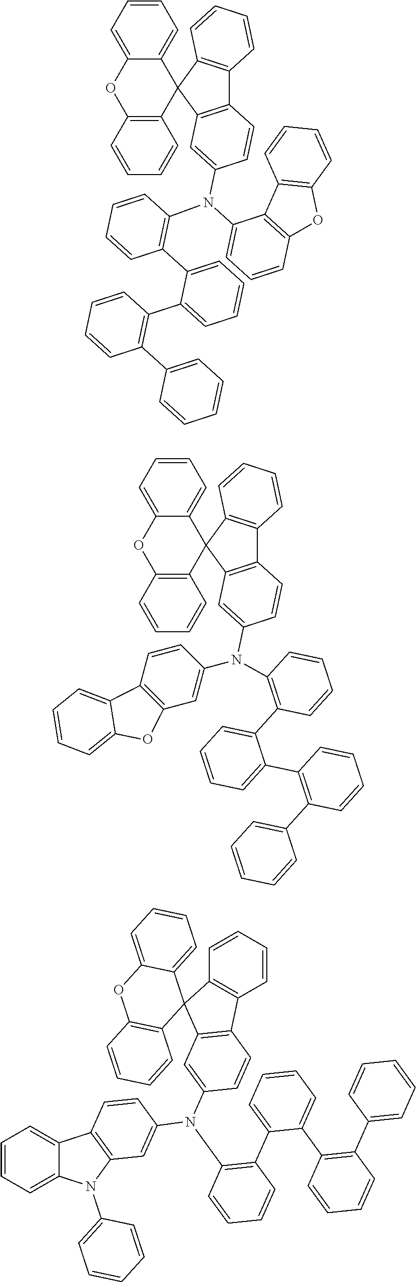

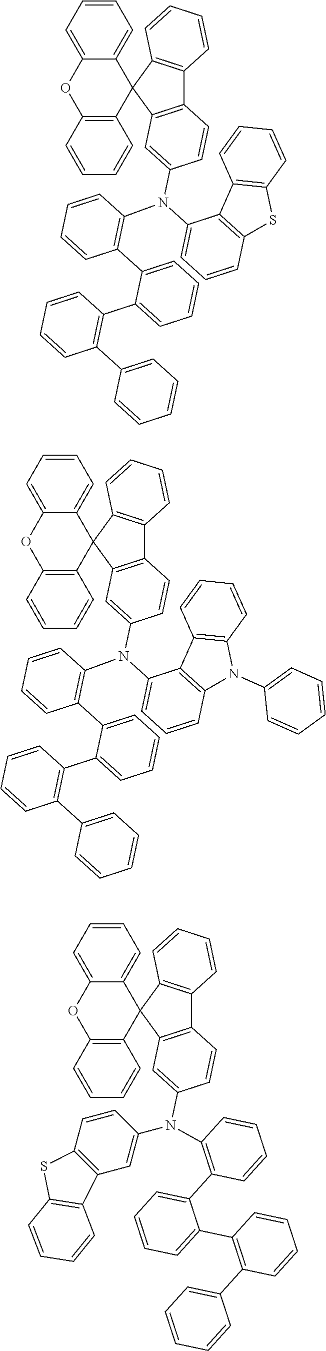

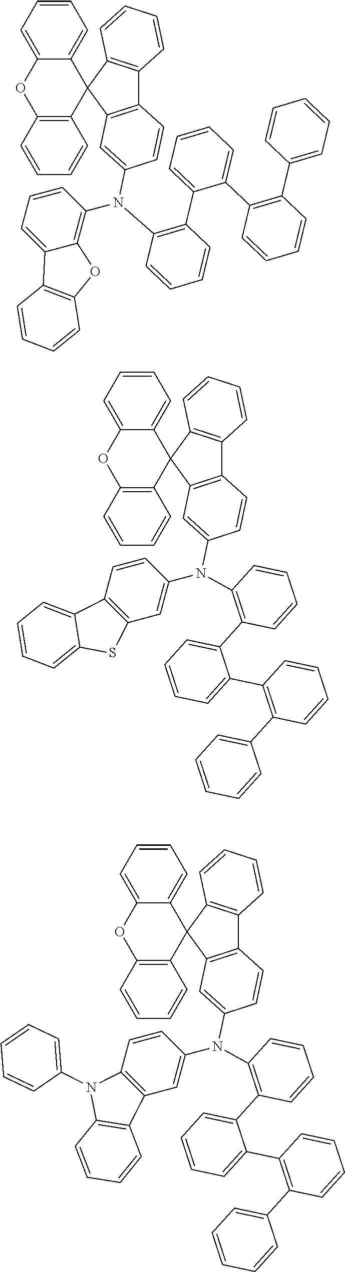 [SMILES: c1ccc(-c2ccccc2-c2ccccc2-c2ccccc2N(c2ccc3c(c2)C2(c4ccccc4Oc4ccccc42)c2ccccc2-3)c2cccc3c2oc2ccccc23)cc1 ; c1ccc(-c2ccccc2-c2ccccc2-c2ccccc2N(c2ccc3c(c2)C2(c4ccccc4Oc4ccccc42)c2ccccc2-3)c2ccc3c(c2)sc2ccccc23)cc1 ; c1ccc(-c2ccccc2-c2ccccc2-c2ccccc2N(c2ccc3c(c2)C2(c4ccccc4Oc4ccccc42)c2ccccc2-3)c2ccc3c(c2)c2ccccc2n3-c2ccccc2)cc1]

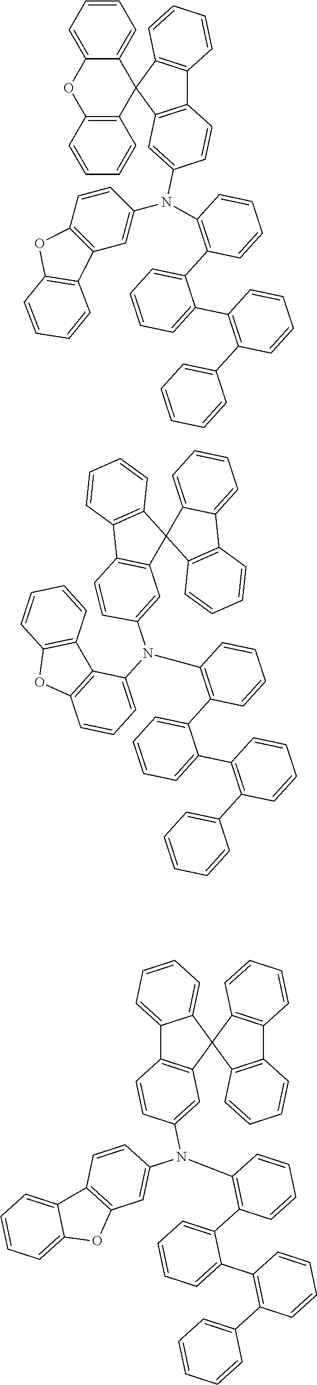

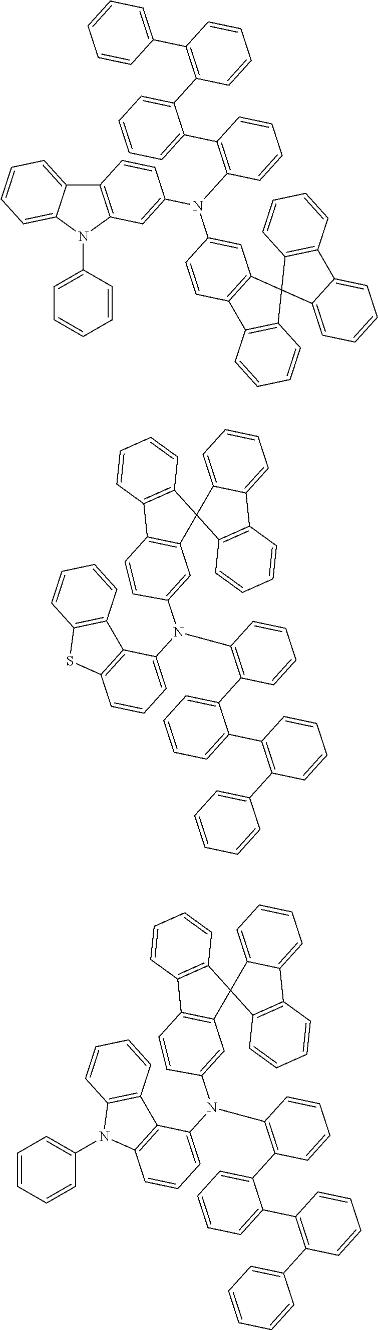 [SMILES: c1ccc(-c2ccccc2-c2ccccc2-c2ccccc2N(c2ccc3c(c2)C2(c4ccccc4-c4ccccc42)c2ccccc2-3)c2ccc3c4ccccc4n(-c4ccccc4)c3c2)cc1 ; c1ccc(-c2ccccc2-c2ccccc2-c2ccccc2N(c2ccc3c(c2)C2(c4ccccc4-c4ccccc42)c2ccccc2-3)c2cccc3sc4ccccc4c23)cc1 ; c1ccc(-c2ccccc2-c2ccccc2-c2ccccc2N(c2ccc3c(c2)C2(c4ccccc4-c4ccccc42)c2ccccc2-3)c2cccc3c2c2ccccc2n3-c2ccccc2)cc1]

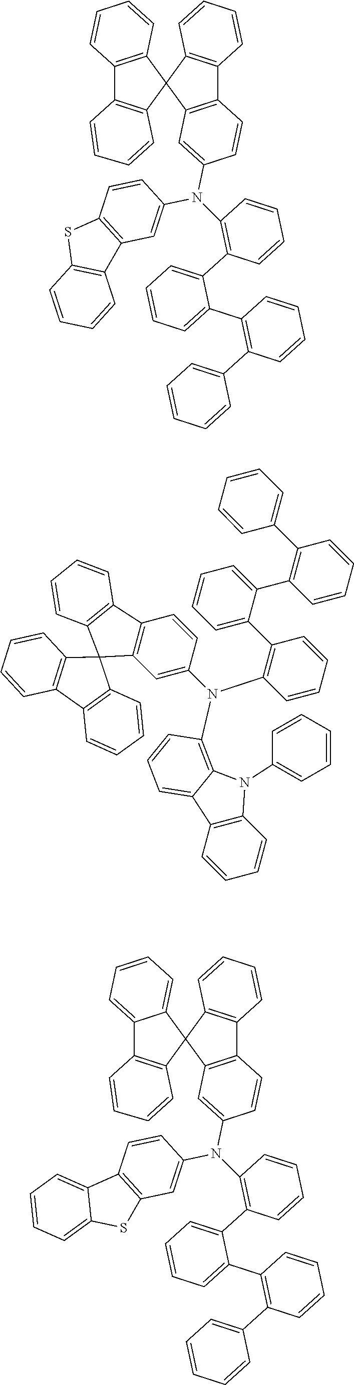 [SMILES: c1ccc(-c2ccccc2-c2ccccc2-c2ccccc2N(c2ccc3c(c2)C2(c4ccccc4-c4ccccc42)c2ccccc2-3)c2ccc3sc4ccccc4c3c2)cc1 ; c1ccc(-c2ccccc2-c2ccccc2-c2ccccc2N(c2ccc3c(c2)C2(c4ccccc4-c4ccccc42)c2ccccc2-3)c2cccc3c4ccccc4n(-c4ccccc4)c23)cc1 ; c1ccc(-c2ccccc2-c2ccccc2-c2ccccc2N(c2ccc3c(c2)C2(c4ccccc4-c4ccccc42)c2ccccc2-3)c2ccc3c(c2)sc2ccccc23)cc1]

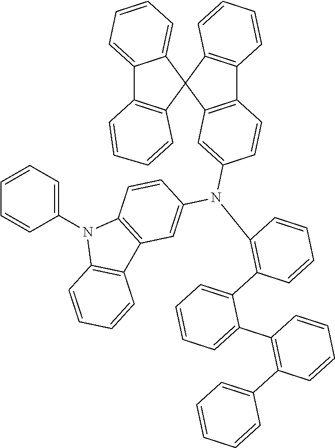 [SMILES: c1ccc(-c2ccccc2-c2ccccc2-c2ccccc2N(c2ccc3c(c2)C2(c4ccccc4-c4ccccc42)c2ccccc2-3)c2ccc3c(c2)c2ccccc2n3-c2ccccc2)cc1]

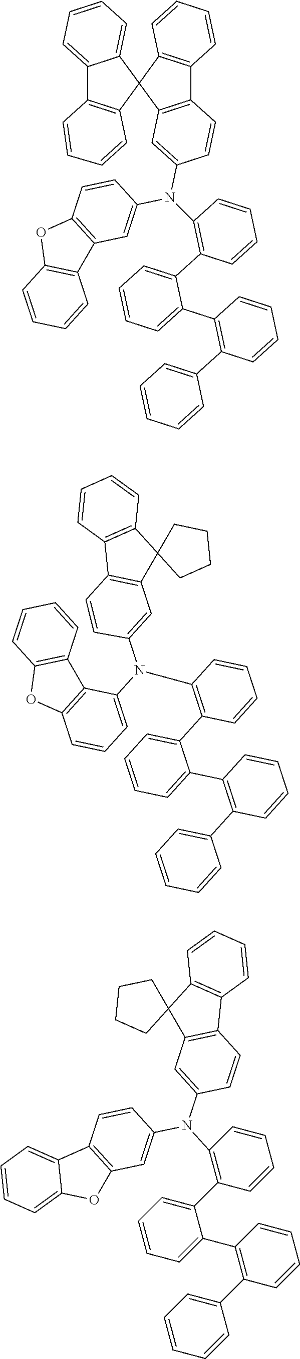

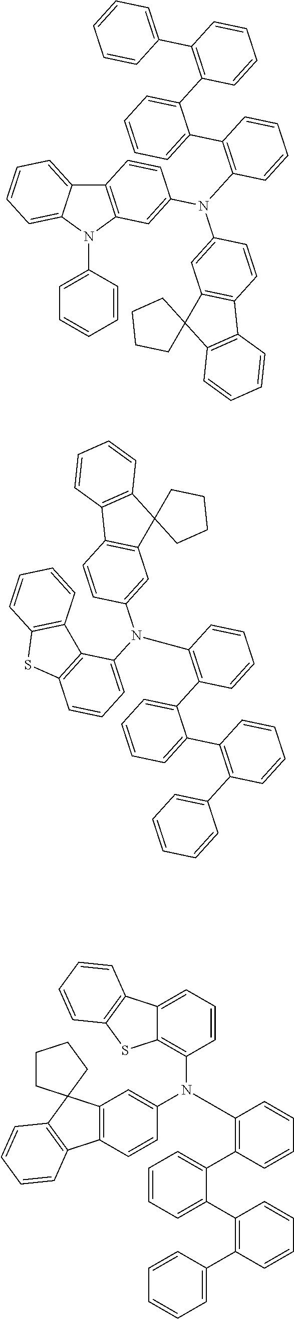 [SMILES: c1ccc(-c2ccccc2-c2ccccc2-c2ccccc2N(c2ccc3c(c2)C2(CCCC2)c2ccccc2-3)c2ccc3c4ccccc4n(-c4ccccc4)c3c2)cc1 ; c1ccc(-c2ccccc2-c2ccccc2-c2ccccc2N(c2ccc3c(c2)C2(CCCC2)c2ccccc2-3)c2cccc3sc4ccccc4c23)cc1 ; c1ccc(-c2ccccc2-c2ccccc2-c2ccccc2N(c2ccc3c(c2)C2(CCCC2)c2ccccc2-3)c2cccc3c2sc2ccccc23)cc1]

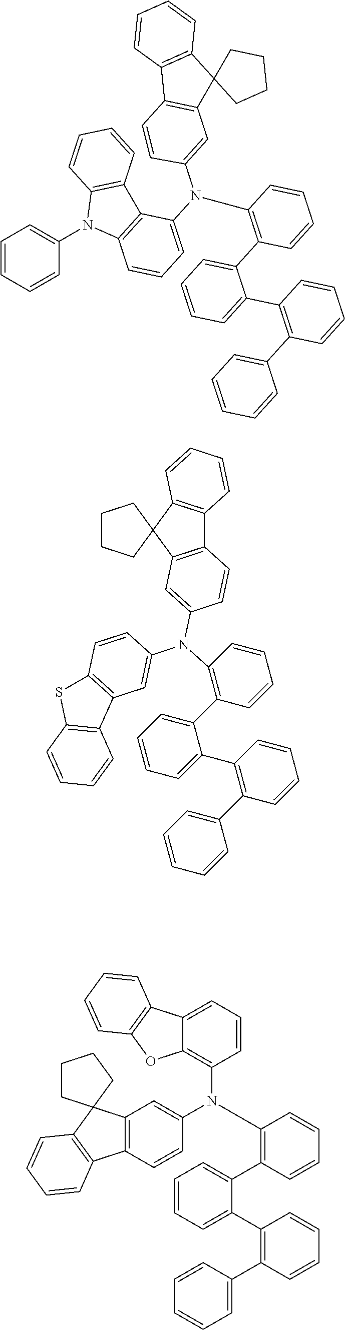 [SMILES: c1ccc(-c2ccccc2-c2ccccc2-c2ccccc2N(c2ccc3c(c2)C2(CCCC2)c2ccccc2-3)c2cccc3c2c2ccccc2n3-c2ccccc2)cc1 ; c1ccc(-c2ccccc2-c2ccccc2-c2ccccc2N(c2ccc3c(c2)C2(CCCC2)c2ccccc2-3)c2ccc3sc4ccccc4c3c2)cc1 ; c1ccc(-c2ccccc2-c2ccccc2-c2ccccc2N(c2ccc3c(c2)C2(CCCC2)c2ccccc2-3)c2cccc3c2oc2ccccc23)cc1]

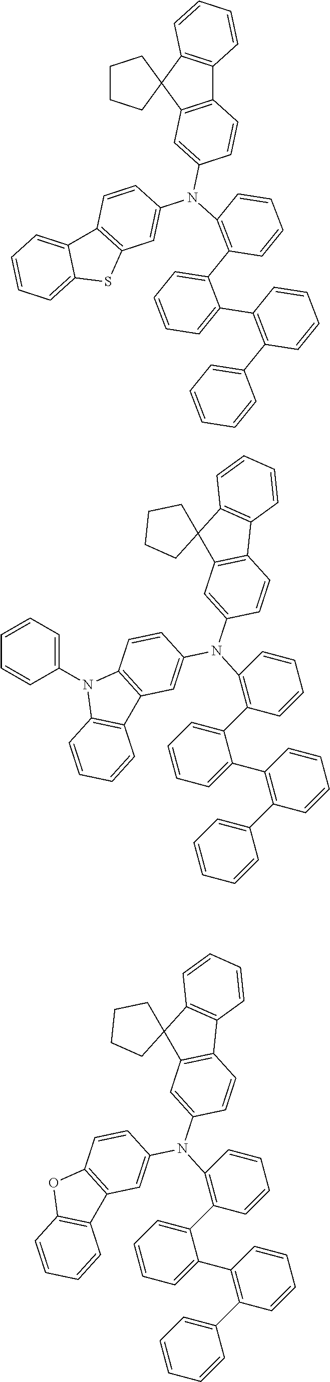

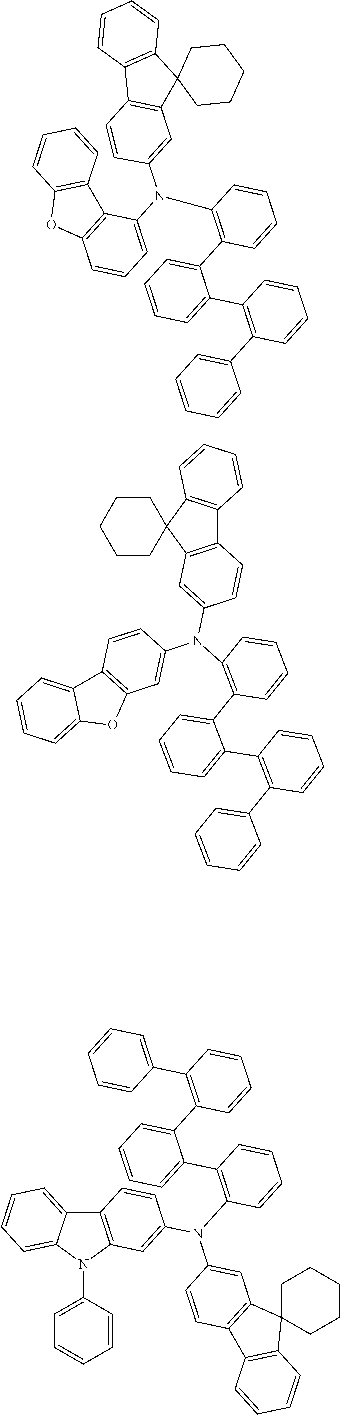

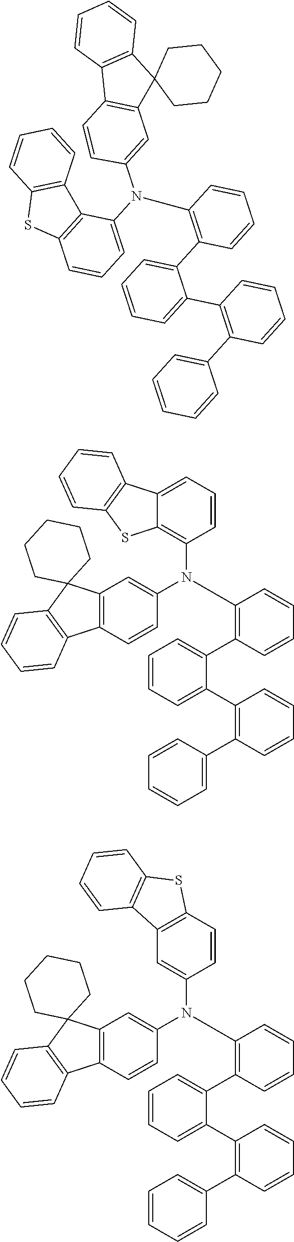 [SMILES: c1ccc(-c2ccccc2-c2ccccc2-c2ccccc2N(c2ccc3c(c2)C2(CCCCC2)c2ccccc2-3)c2cccc3sc4ccccc4c23)cc1 ; c1ccc(-c2ccccc2-c2ccccc2-c2ccccc2N(c2ccc3c(c2)C2(CCCCC2)c2ccccc2-3)c2cccc3c2sc2ccccc23)cc1 ; c1ccc(-c2ccccc2-c2ccccc2-c2ccccc2N(c2ccc3c(c2)C2(CCCCC2)c2ccccc2-3)c2ccc3sc4ccccc4c3c2)cc1]

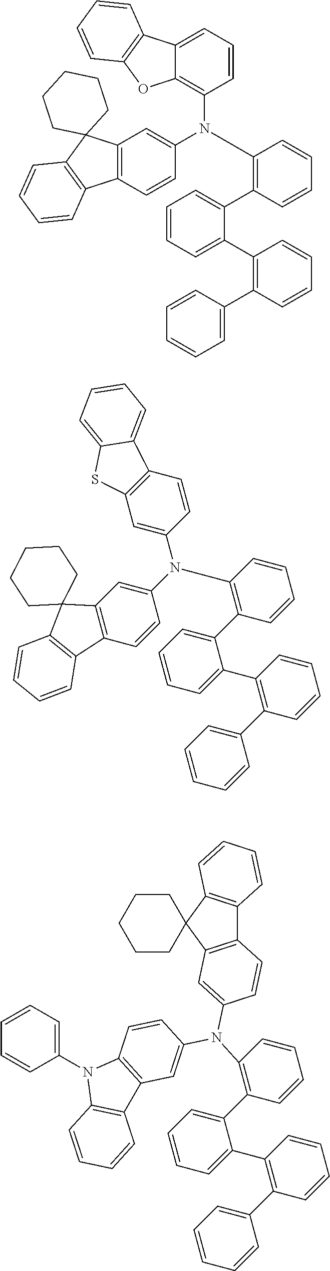 [SMILES: c1ccc(-c2ccccc2-c2ccccc2-c2ccccc2N(c2ccc3c(c2)C2(CCCCC2)c2ccccc2-3)c2cccc3c2oc2ccccc23)cc1 ; c1ccc(-c2ccccc2-c2ccccc2-c2ccccc2N(c2ccc3c(c2)C2(CCCCC2)c2ccccc2-3)c2ccc3c(c2)sc2ccccc23)cc1 ; c1ccc(-c2ccccc2-c2ccccc2-c2ccccc2N(c2ccc3c(c2)C2(CCCCC2)c2ccccc2-3)c2ccc3c(c2)c2ccccc2n3-c2ccccc2)cc1]

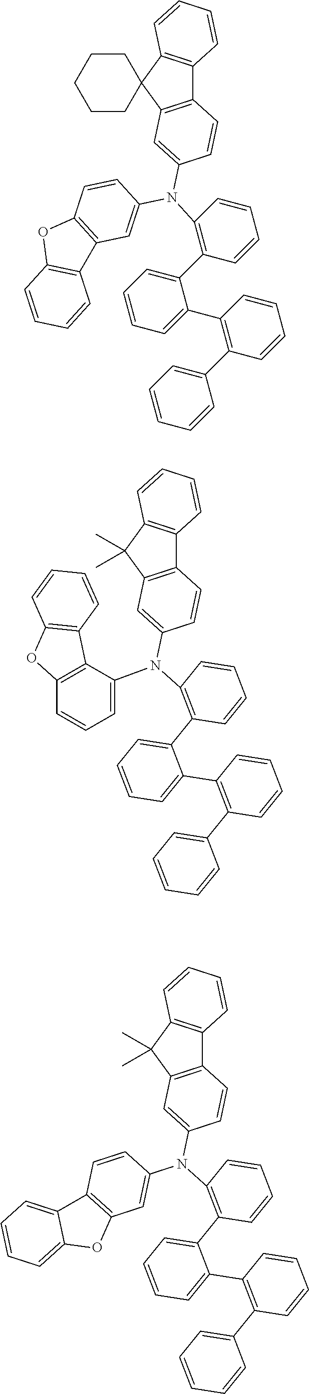 [SMILES: c1ccc(-c2ccccc2-c2ccccc2-c2ccccc2N(c2ccc3c(c2)C2(CCCCC2)c2ccccc2-3)c2ccc3oc4ccccc4c3c2)cc1 ; CC1(C)c2ccccc2-c2ccc(N(c3ccccc3-c3ccccc3-c3ccccc3-c3ccccc3)c3cccc4oc5ccccc5c34)cc21 ; CC1(C)c2ccccc2-c2ccc(N(c3ccc4c(c3)oc3ccccc34)c3ccccc3-c3ccccc3-c3ccccc3-c3ccccc3)cc21]

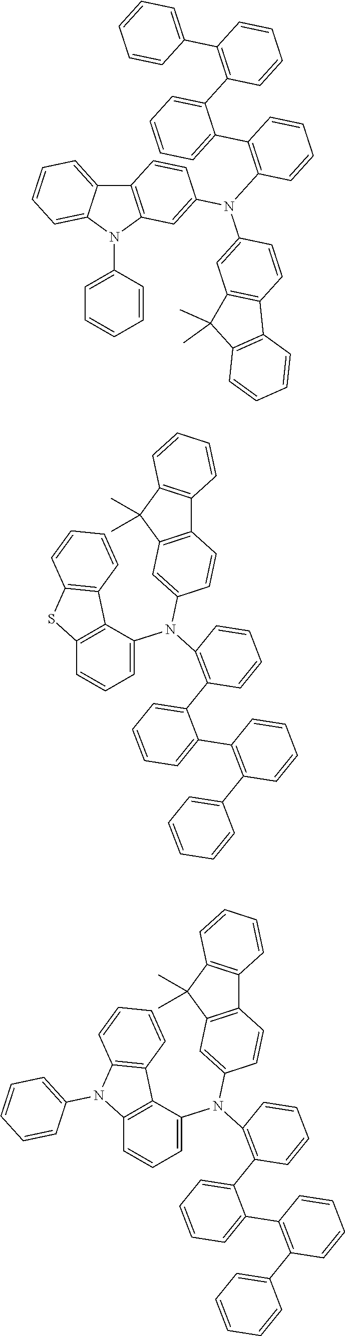 [SMILES: CC1(C)c2ccccc2-c2ccc(N(c3ccc4c5ccccc5n(-c5ccccc5)c4c3)c3ccccc3-c3ccccc3-c3ccccc3-c3ccccc3)cc21 ; CC1(C)c2ccccc2-c2ccc(N(c3ccccc3-c3ccccc3-c3ccccc3-c3ccccc3)c3cccc4sc5ccccc5c34)cc21 ; CC1(C)c2ccccc2-c2ccc(N(c3ccccc3-c3ccccc3-c3ccccc3-c3ccccc3)c3cccc4c3c3ccccc3n4-c3ccccc3)cc21]

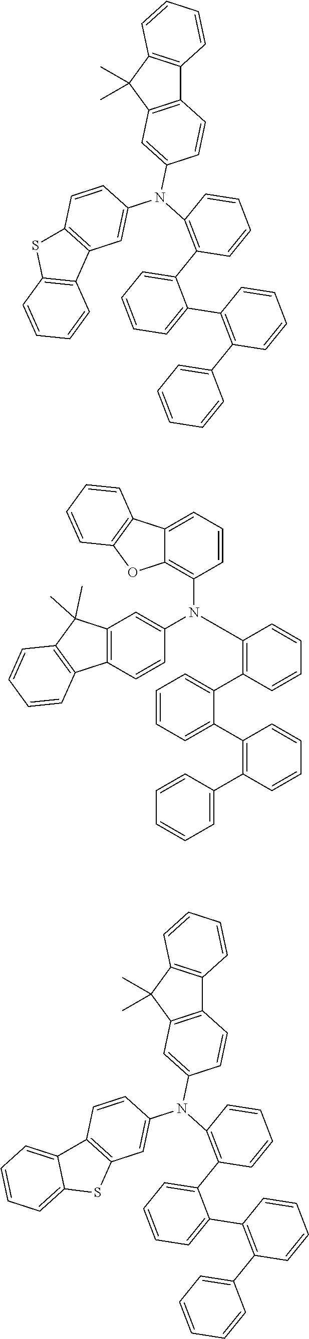 [SMILES: CC1(C)c2ccccc2-c2ccc(N(c3ccc4sc5ccccc5c4c3)c3ccccc3-c3ccccc3-c3ccccc3-c3ccccc3)cc21 ; CC1(C)c2ccccc2-c2ccc(N(c3ccccc3-c3ccccc3-c3ccccc3-c3ccccc3)c3cccc4c3oc3ccccc34)cc21 ; CC1(C)c2ccccc2-c2ccc(N(c3ccc4c(c3)sc3ccccc34)c3ccccc3-c3ccccc3-c3ccccc3-c3ccccc3)cc21]

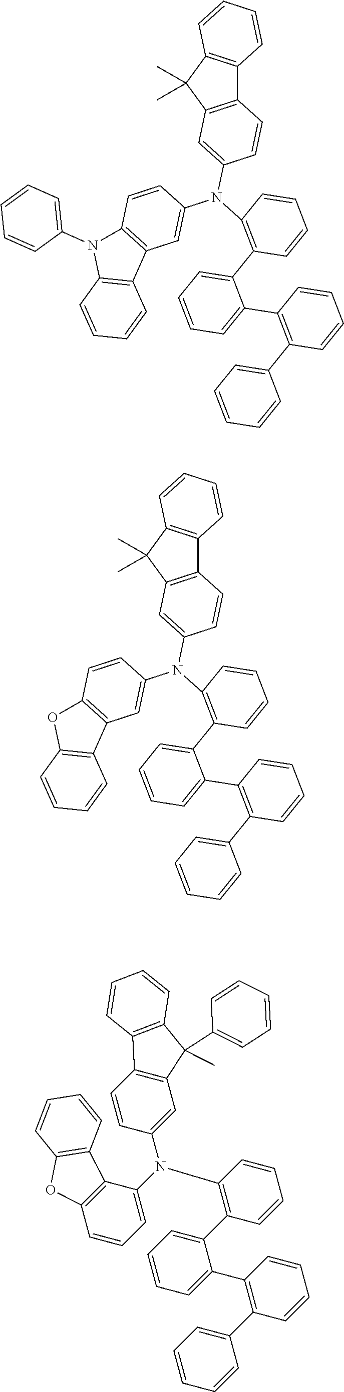 [SMILES: CC1(C)c2ccccc2-c2ccc(N(c3ccc4c(c3)c3ccccc3n4-c3ccccc3)c3ccccc3-c3ccccc3-c3ccccc3-c3ccccc3)cc21 ; CC1(C)c2ccccc2-c2ccc(N(c3ccc4oc5ccccc5c4c3)c3ccccc3-c3ccccc3-c3ccccc3-c3ccccc3)cc21 ; CC1(c2ccccc2)c2ccccc2-c2ccc(N(c3ccccc3-c3ccccc3-c3ccccc3-c3ccccc3)c3cccc4oc5ccccc5c34)cc21]

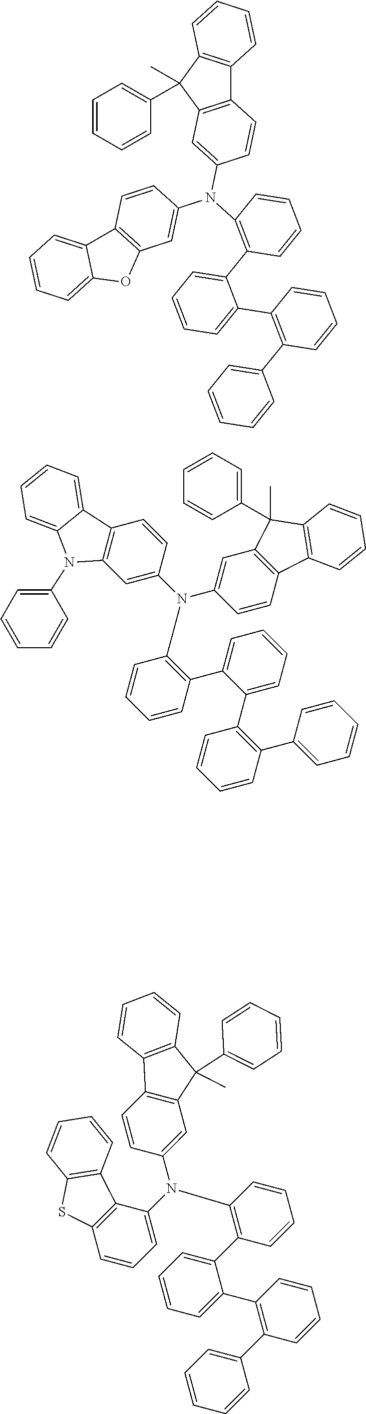

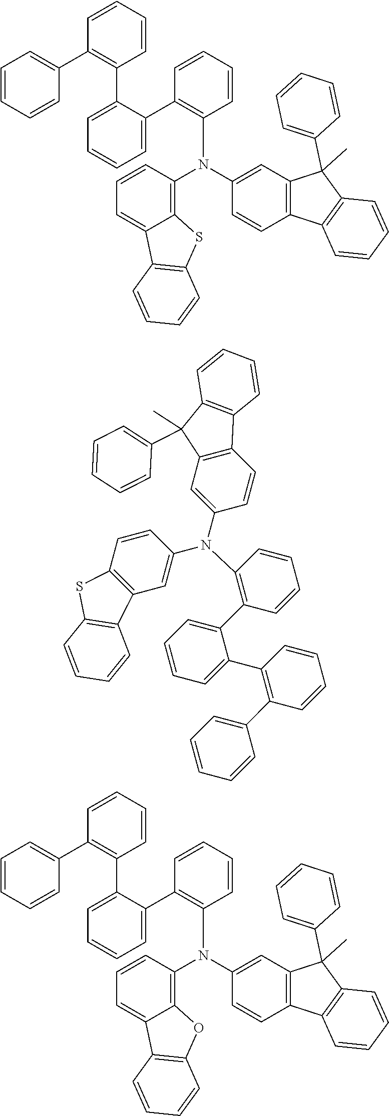 [SMILES: CC1(c2ccccc2)c2ccccc2-c2ccc(N(c3ccccc3-c3ccccc3-c3ccccc3-c3ccccc3)c3cccc4c3sc3ccccc34)cc21 ; CC1(c2ccccc2)c2ccccc2-c2ccc(N(c3ccc4sc5ccccc5c4c3)c3ccccc3-c3ccccc3-c3ccccc3-c3ccccc3)cc21 ; CC1(c2ccccc2)c2ccccc2-c2ccc(N(c3ccccc3-c3ccccc3-c3ccccc3-c3ccccc3)c3cccc4c3oc3ccccc34)cc21]

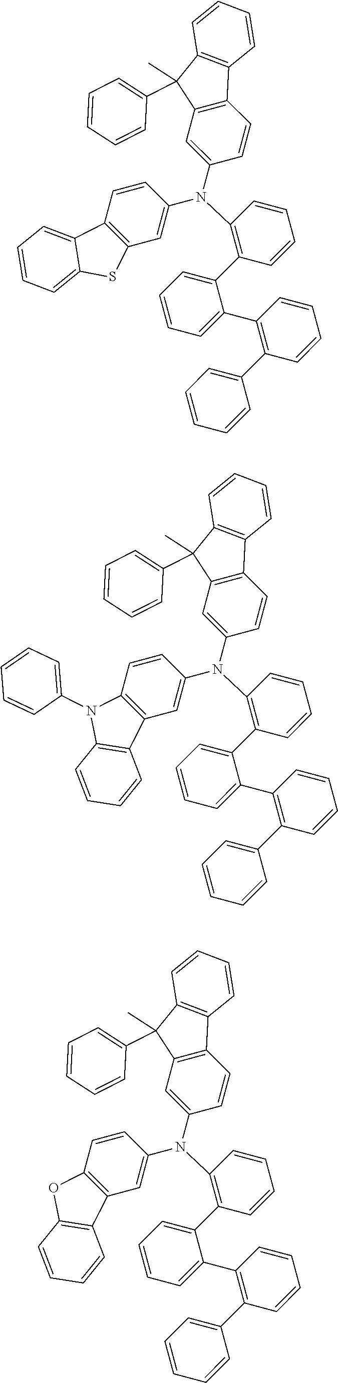

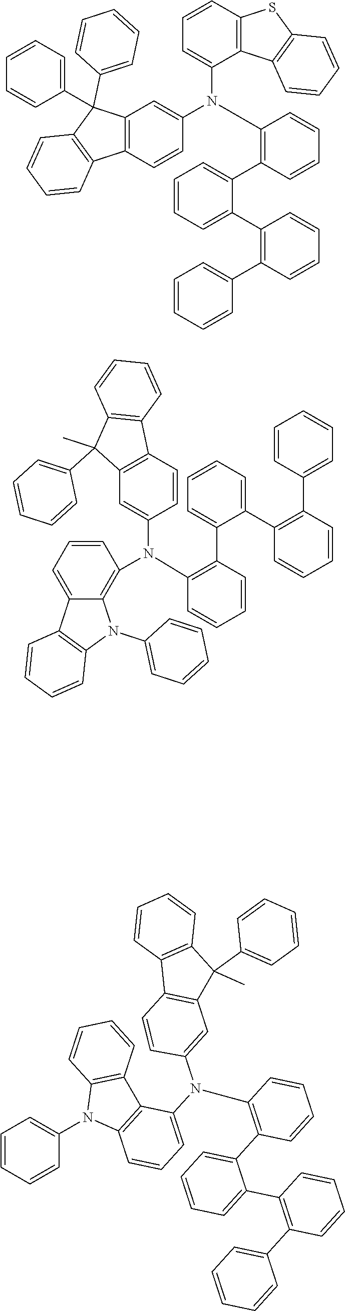 [SMILES: c1ccc(-c2ccccc2-c2ccccc2-c2ccccc2N(c2ccc3c(c2)C(c2ccccc2)(c2ccccc2)c2ccccc2-3)c2cccc3sc4ccccc4c23)cc1 ; CC1(c2ccccc2)c2ccccc2-c2ccc(N(c3ccccc3-c3ccccc3-c3ccccc3-c3ccccc3)c3cccc4c5ccccc5n(-c5ccccc5)c34)cc21 ; CC1(c2ccccc2)c2ccccc2-c2ccc(N(c3ccccc3-c3ccccc3-c3ccccc3-c3ccccc3)c3cccc4c3c3ccccc3n4-c3ccccc3)cc21]

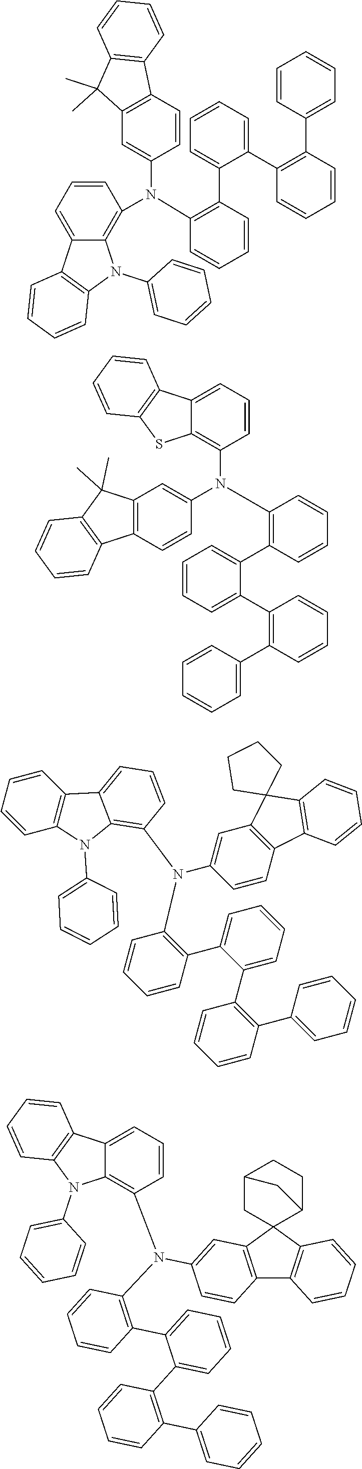

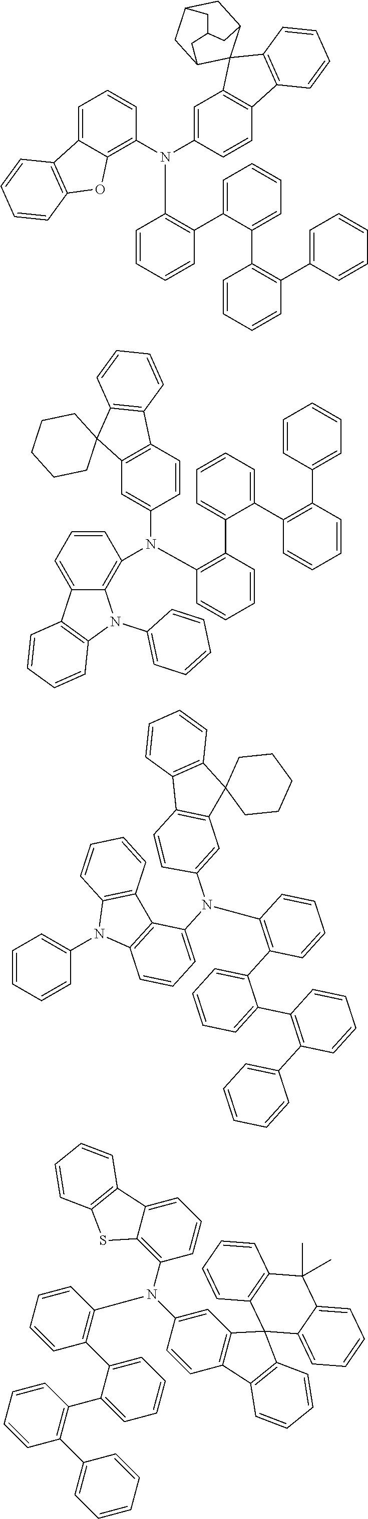

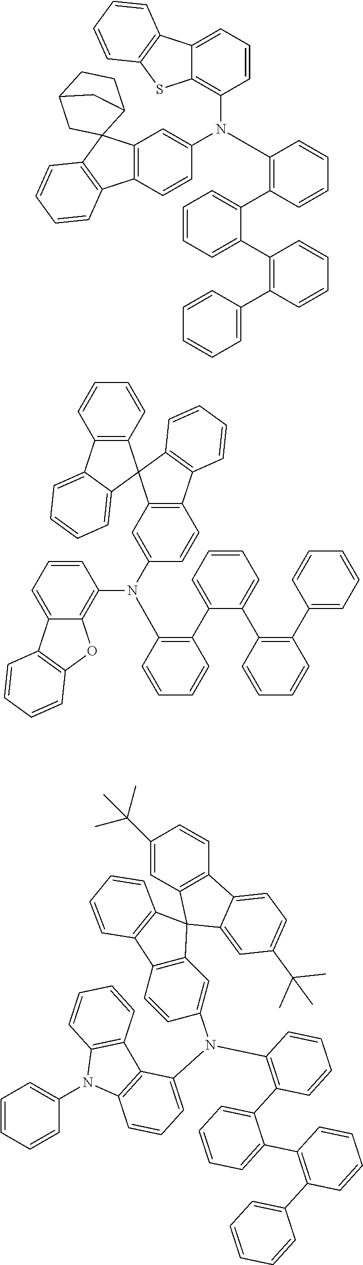 [SMILES: c1ccc(-c2ccccc2-c2ccccc2-c2ccccc2N(c2ccc3c(c2)C2(CC4CCC2C4)c2ccccc2-3)c2cccc3c2sc2ccccc23)cc1 ; c1ccc(-c2ccccc2-c2ccccc2-c2ccccc2N(c2ccc3c(c2)C2(c4ccccc4-c4ccccc42)c2ccccc2-3)c2cccc3c2oc2ccccc23)cc1 ; CC(C)(C)c1ccc2c(c1)C1(c3ccccc3-c3ccc(N(c4ccccc4-c4ccccc4-c4ccccc4-c4ccccc4)c4cccc5c4c4ccccc4n5-c4ccccc4)cc31)c1cc(C(C)(C)C)ccc1-2]

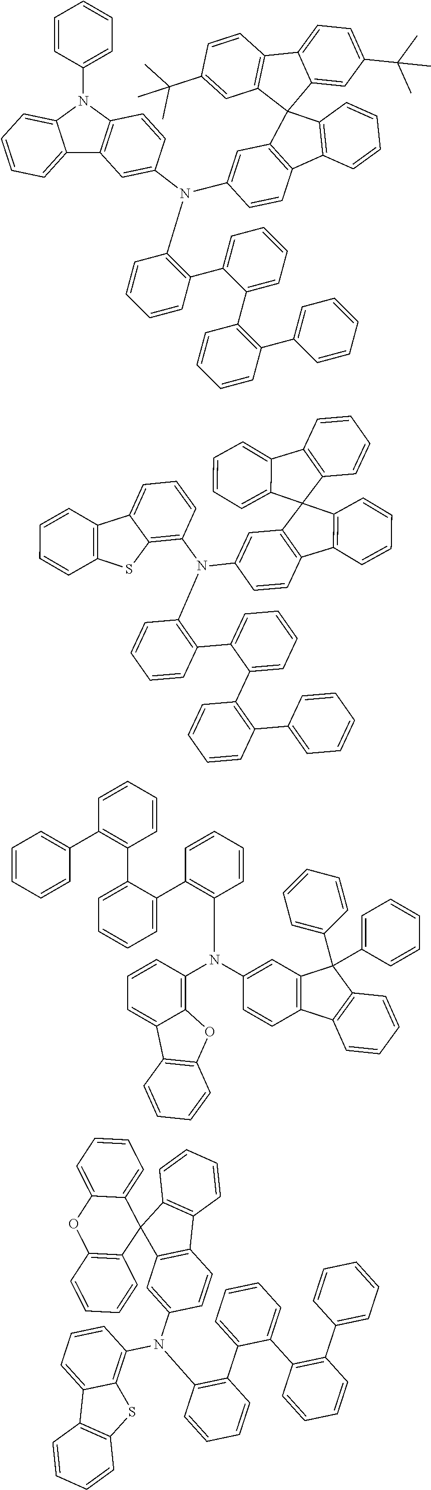

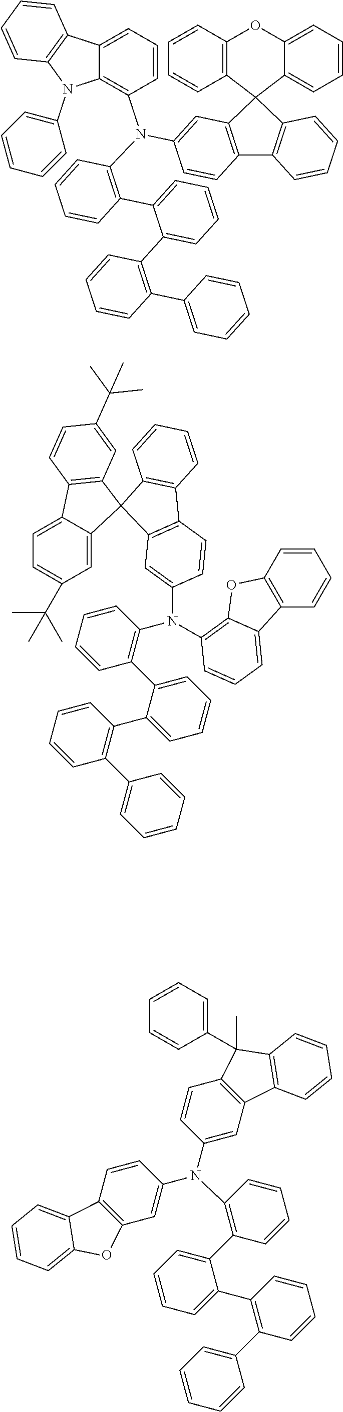

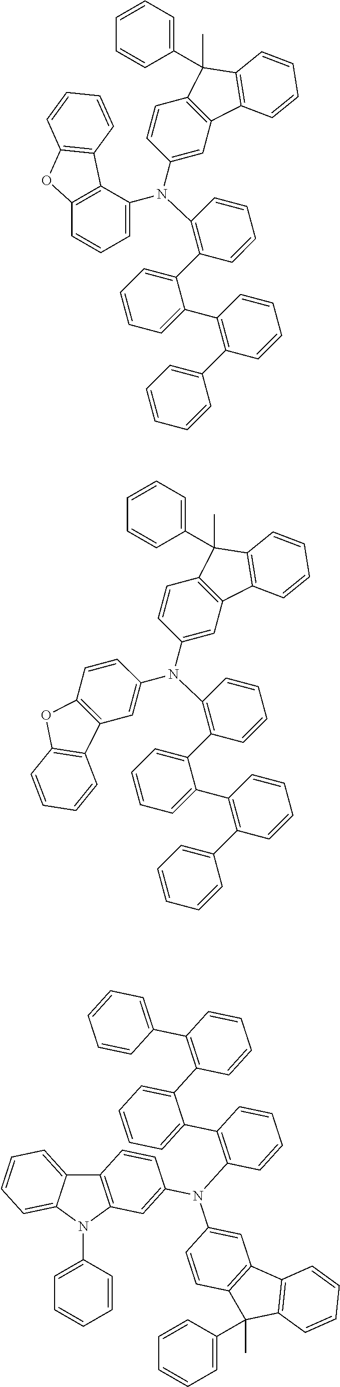 [SMILES: CC1(c2ccccc2)c2ccccc2-c2cc(N(c3ccccc3-c3ccccc3-c3ccccc3-c3ccccc3)c3cccc4oc5ccccc5c34)ccc21 ; CC1(c2ccccc2)c2ccccc2-c2cc(N(c3ccc4oc5ccccc5c4c3)c3ccccc3-c3ccccc3-c3ccccc3-c3ccccc3)ccc21 ; CC1(c2ccccc2)c2ccccc2-c2cc(N(c3ccc4c5ccccc5n(-c5ccccc5)c4c3)c3ccccc3-c3ccccc3-c3ccccc3-c3ccccc3)ccc21]

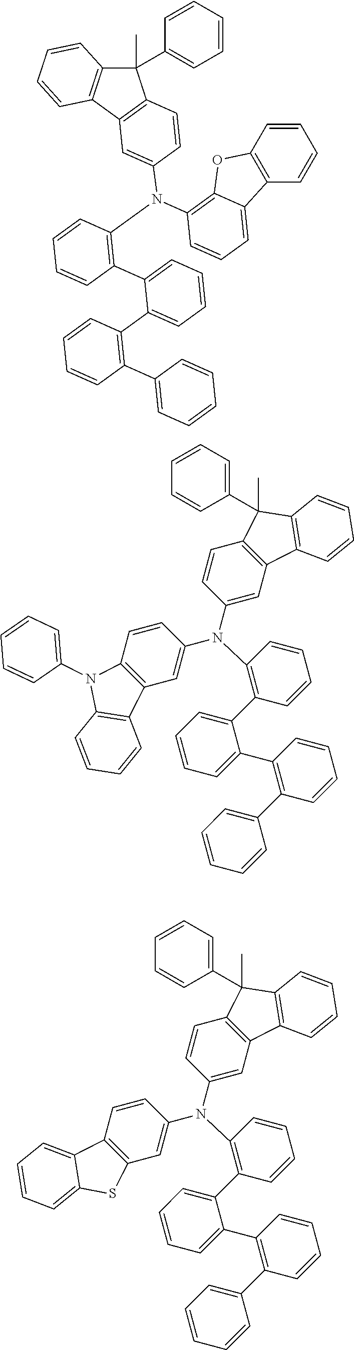

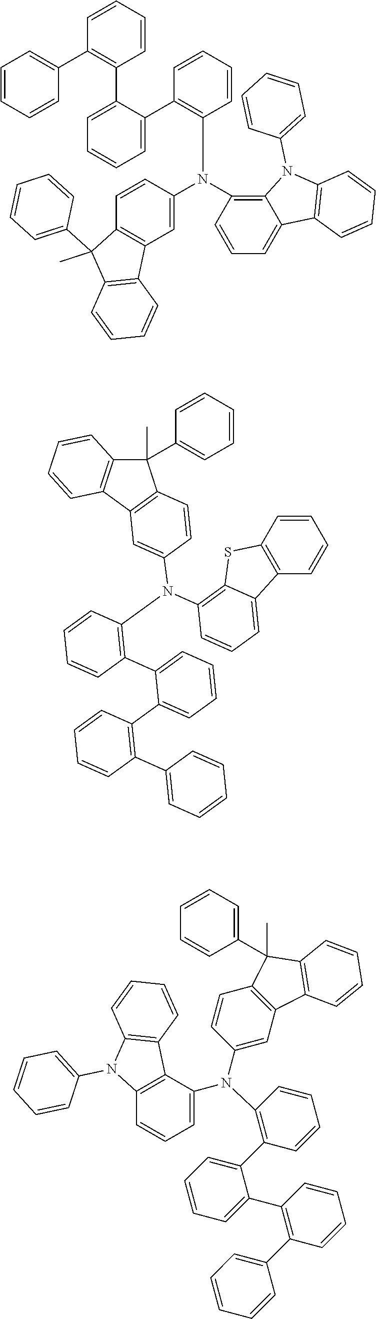 [SMILES: CC1(c2ccccc2)c2ccccc2-c2cc(N(c3ccccc3-c3ccccc3-c3ccccc3-c3ccccc3)c3cccc4c5ccccc5n(-c5ccccc5)c34)ccc21 ; CC1(c2ccccc2)c2ccccc2-c2cc(N(c3ccccc3-c3ccccc3-c3ccccc3-c3ccccc3)c3cccc4c3sc3ccccc34)ccc21 ; CC1(c2ccccc2)c2ccccc2-c2cc(N(c3ccccc3-c3ccccc3-c3ccccc3-c3ccccc3)c3cccc4c3c3ccccc3n4-c3ccccc3)ccc21]

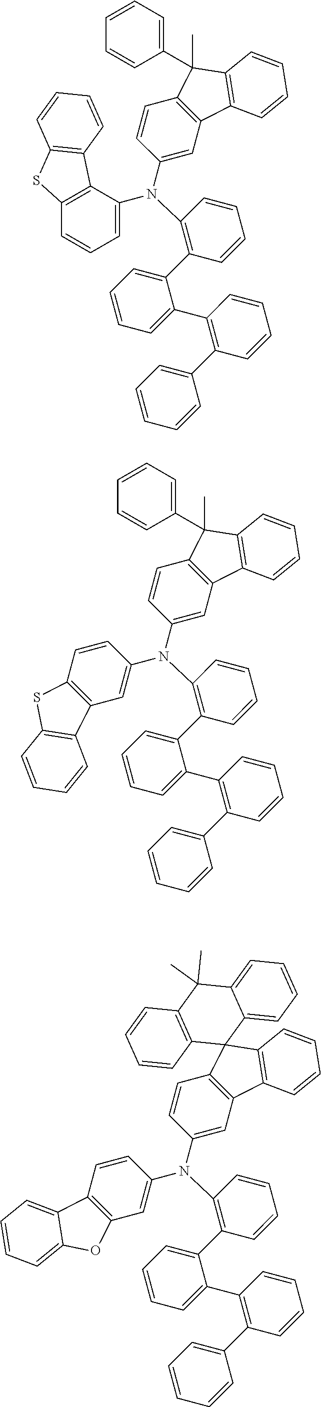

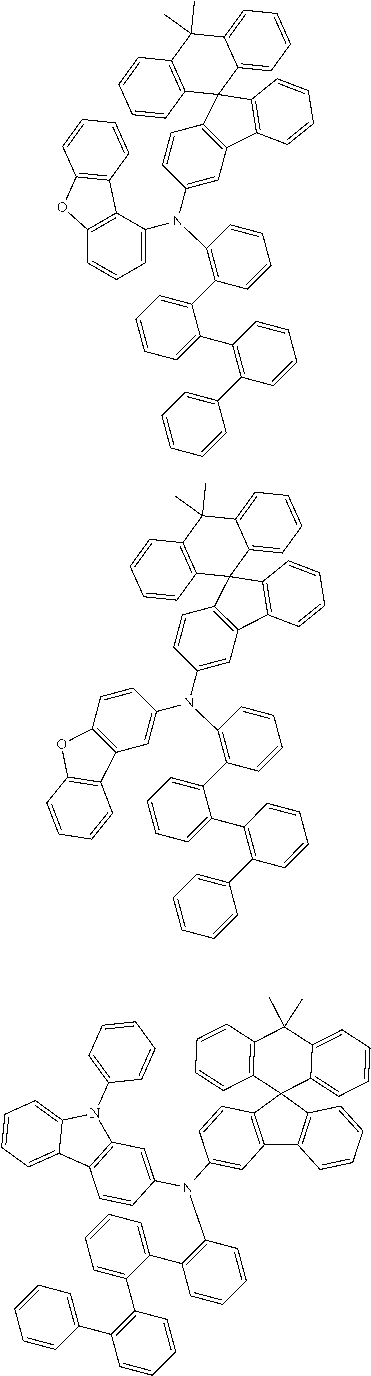

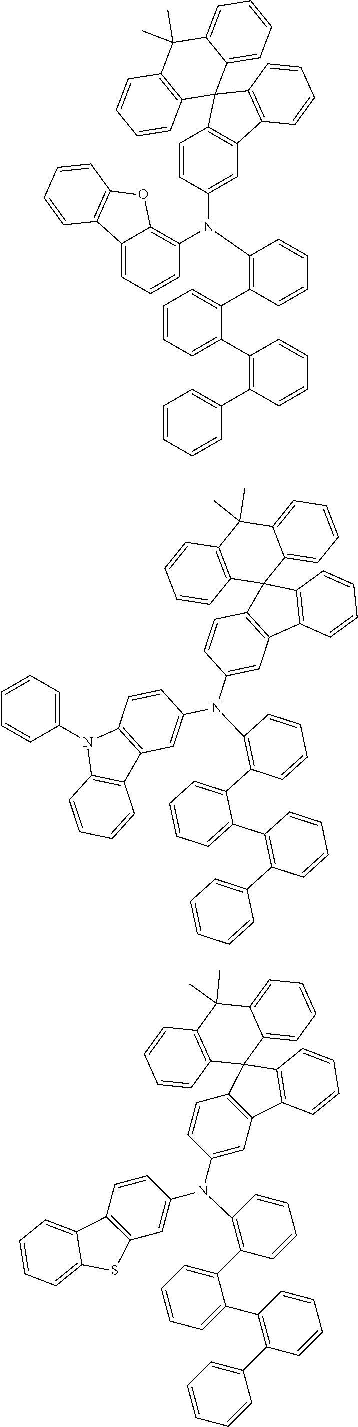

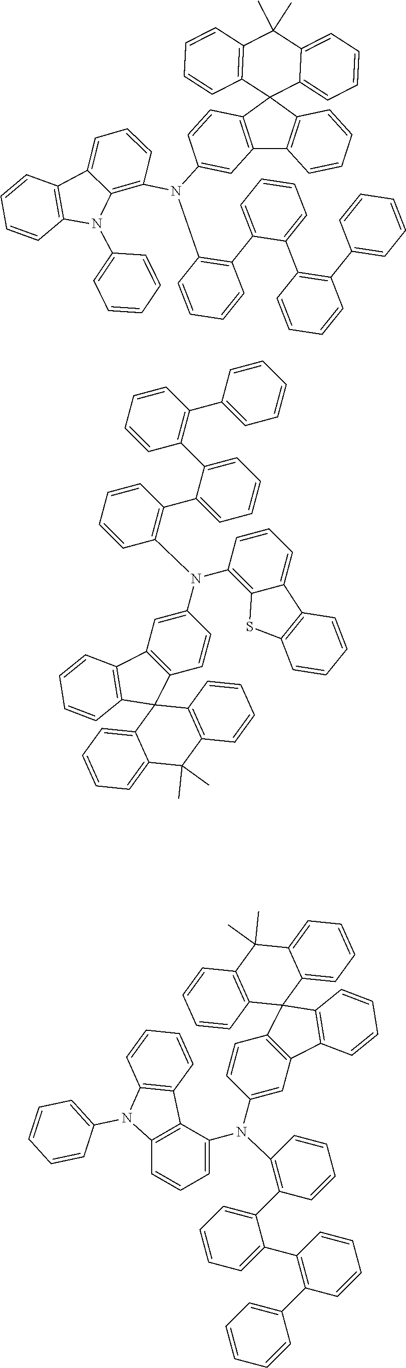

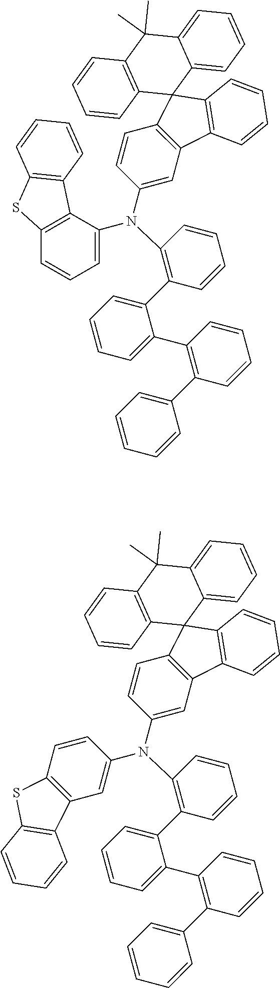 [SMILES: CC1(C)c2ccccc2C2(c3ccccc3-c3cc(N(c4ccccc4-c4ccccc4-c4ccccc4-c4ccccc4)c4cccc5sc6ccccc6c45)ccc32)c2ccccc21 ; CC1(C)c2ccccc2C2(c3ccccc3-c3cc(N(c4ccc5sc6ccccc6c5c4)c4ccccc4-c4ccccc4-c4ccccc4-c4ccccc4)ccc32)c2ccccc21]

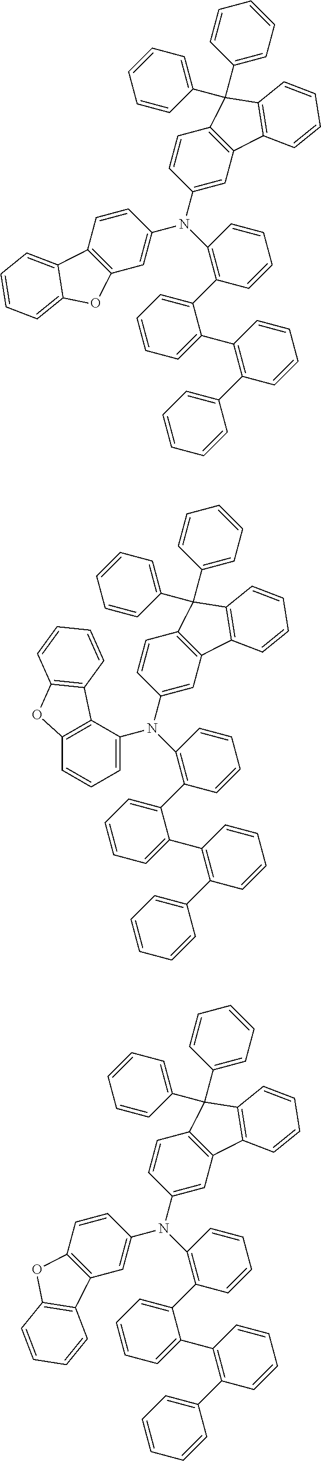 [SMILES: c1ccc(-c2ccccc2-c2ccccc2-c2ccccc2N(c2ccc3c(c2)-c2ccccc2C3(c2ccccc2)c2ccccc2)c2ccc3c(c2)oc2ccccc23)cc1 ; c1ccc(-c2ccccc2-c2ccccc2-c2ccccc2N(c2ccc3c(c2)-c2ccccc2C3(c2ccccc2)c2ccccc2)c2cccc3oc4ccccc4c23)cc1 ; c1ccc(-c2ccccc2-c2ccccc2-c2ccccc2N(c2ccc3c(c2)-c2ccccc2C3(c2ccccc2)c2ccccc2)c2ccc3oc4ccccc4c3c2)cc1]

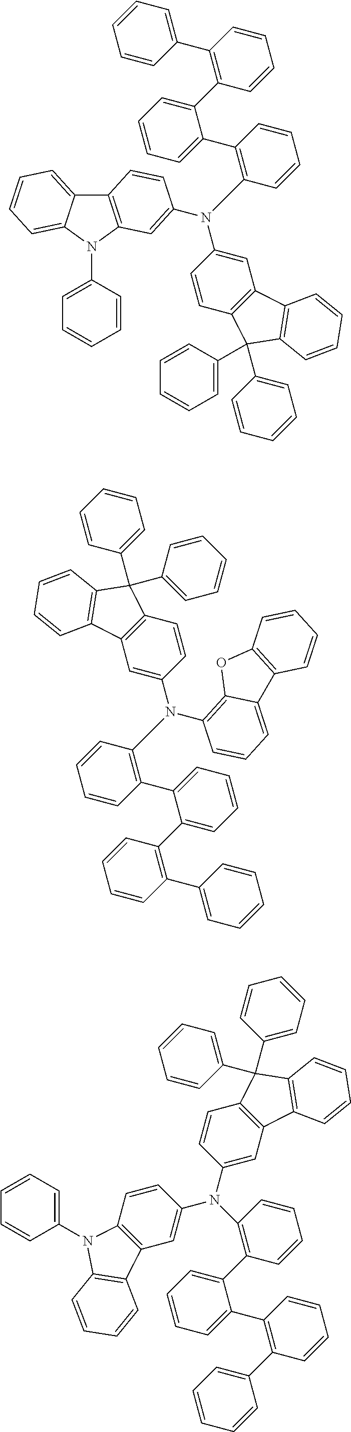 [SMILES: c1ccc(-c2ccccc2-c2ccccc2-c2ccccc2N(c2ccc3c(c2)-c2ccccc2C3(c2ccccc2)c2ccccc2)c2ccc3c4ccccc4n(-c4ccccc4)c3c2)cc1 ; c1ccc(-c2ccccc2-c2ccccc2-c2ccccc2N(c2ccc3c(c2)-c2ccccc2C3(c2ccccc2)c2ccccc2)c2cccc3c2oc2ccccc23)cc1 ; c1ccc(-c2ccccc2-c2ccccc2-c2ccccc2N(c2ccc3c(c2)-c2ccccc2C3(c2ccccc2)c2ccccc2)c2ccc3c(c2)c2ccccc2n3-c2ccccc2)cc1]

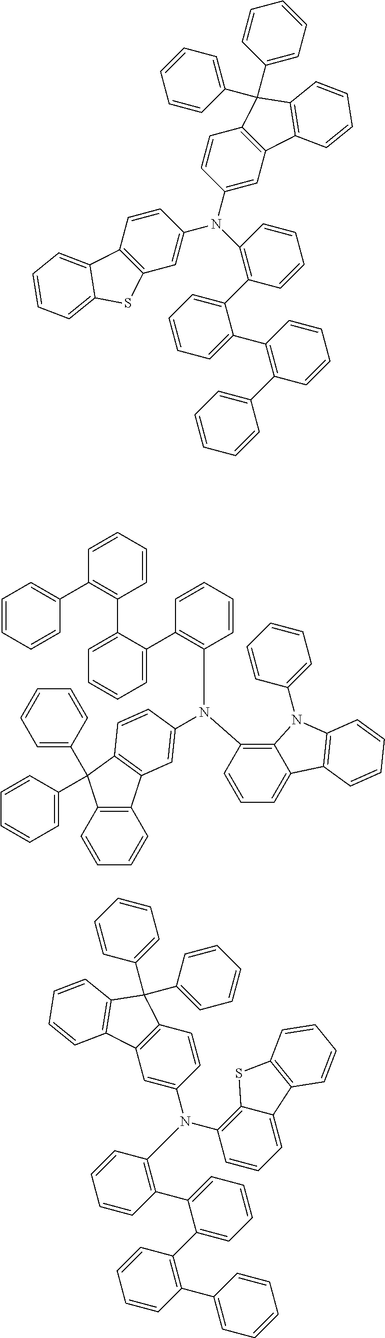

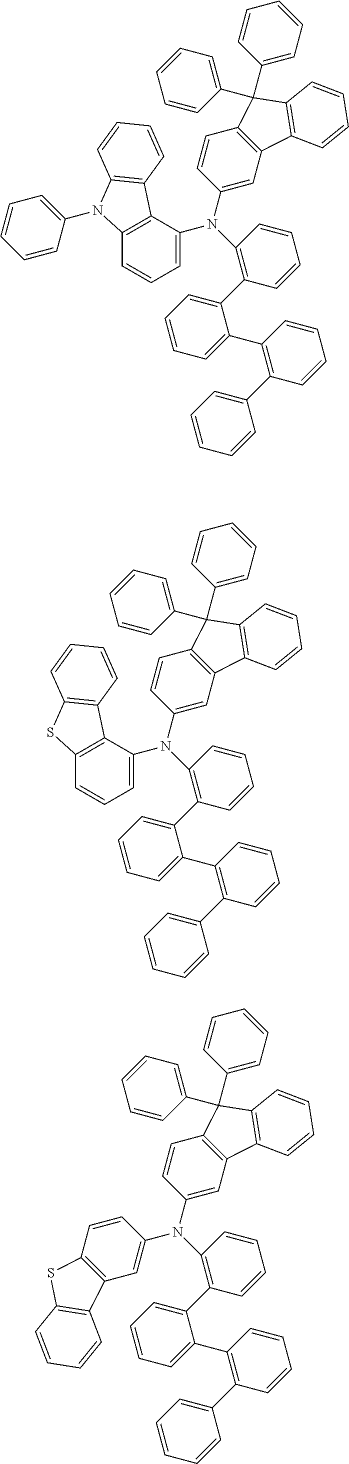 [SMILES: c1ccc(-c2ccccc2-c2ccccc2-c2ccccc2N(c2ccc3c(c2)-c2ccccc2C3(c2ccccc2)c2ccccc2)c2cccc3c2c2ccccc2n3-c2ccccc2)cc1 ; c1ccc(-c2ccccc2-c2ccccc2-c2ccccc2N(c2ccc3c(c2)-c2ccccc2C3(c2ccccc2)c2ccccc2)c2cccc3sc4ccccc4c23)cc1 ; c1ccc(-c2ccccc2-c2ccccc2-c2ccccc2N(c2ccc3c(c2)-c2ccccc2C3(c2ccccc2)c2ccccc2)c2ccc3sc4ccccc4c3c2)cc1]

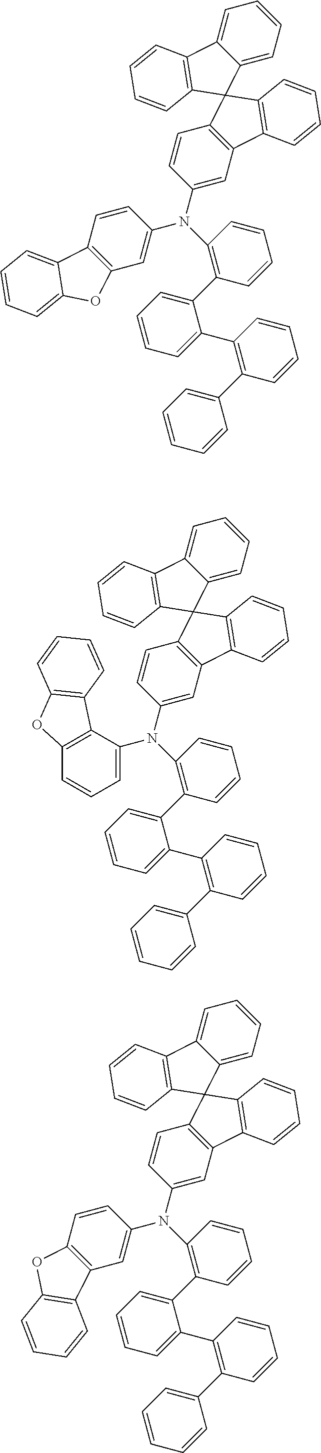 [SMILES: c1ccc(-c2ccccc2-c2ccccc2-c2ccccc2N(c2ccc3c(c2)-c2ccccc2C32c3ccccc3-c3ccccc32)c2ccc3c(c2)oc2ccccc23)cc1 ; c1ccc(-c2ccccc2-c2ccccc2-c2ccccc2N(c2ccc3c(c2)-c2ccccc2C32c3ccccc3-c3ccccc32)c2cccc3oc4ccccc4c23)cc1 ; c1ccc(-c2ccccc2-c2ccccc2-c2ccccc2N(c2ccc3c(c2)-c2ccccc2C32c3ccccc3-c3ccccc32)c2ccc3oc4ccccc4c3c2)cc1]

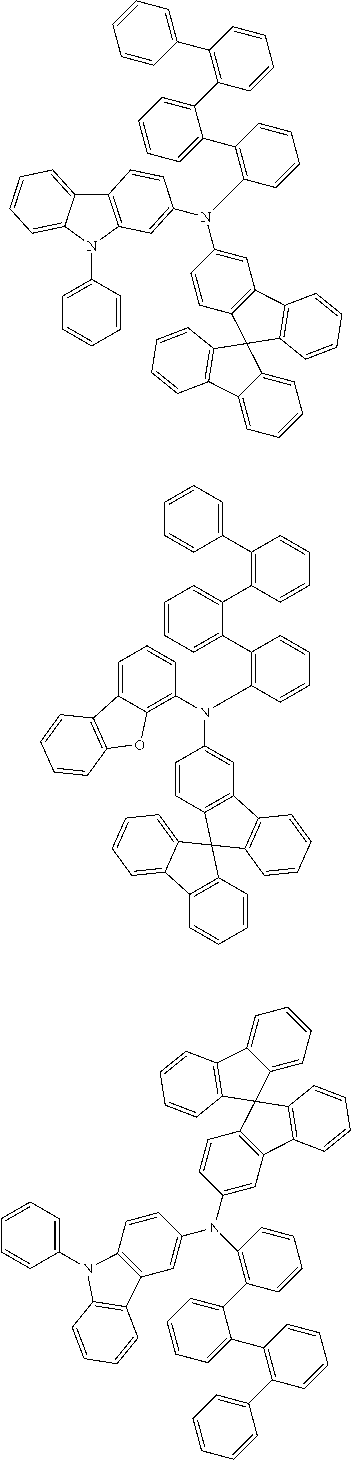 [SMILES: c1ccc(-c2ccccc2-c2ccccc2-c2ccccc2N(c2ccc3c(c2)-c2ccccc2C32c3ccccc3-c3ccccc32)c2ccc3c4ccccc4n(-c4ccccc4)c3c2)cc1 ; c1ccc(-c2ccccc2-c2ccccc2-c2ccccc2N(c2ccc3c(c2)-c2ccccc2C32c3ccccc3-c3ccccc32)c2cccc3c2oc2ccccc23)cc1 ; c1ccc(-c2ccccc2-c2ccccc2-c2ccccc2N(c2ccc3c(c2)-c2ccccc2C32c3ccccc3-c3ccccc32)c2ccc3c(c2)c2ccccc2n3-c2ccccc2)cc1]

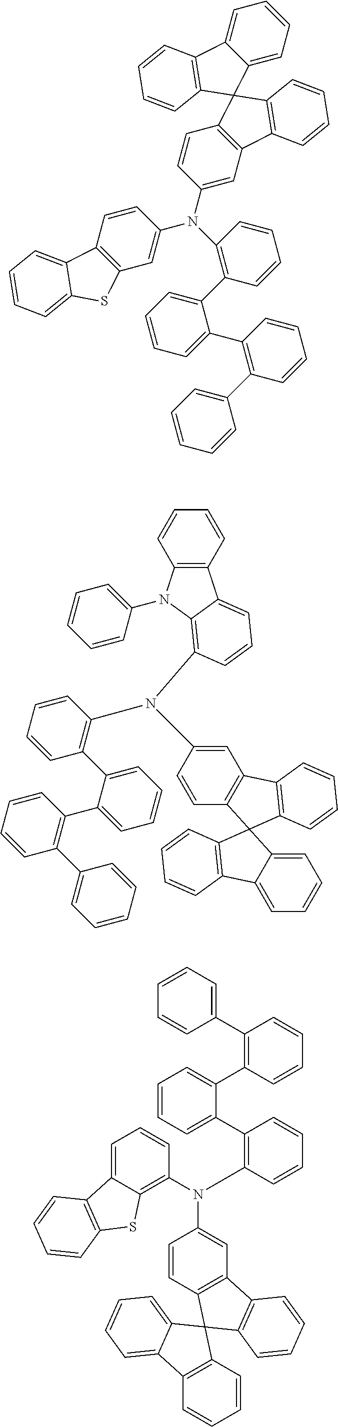 [SMILES: c1ccc(-c2ccccc2-c2ccccc2-c2ccccc2N(c2ccc3c(c2)-c2ccccc2C32c3ccccc3-c3ccccc32)c2ccc3c(c2)sc2ccccc23)cc1 ; c1ccc(-c2ccccc2-c2ccccc2-c2ccccc2N(c2ccc3c(c2)-c2ccccc2C32c3ccccc3-c3ccccc32)c2cccc3c4ccccc4n(-c4ccccc4)c23)cc1 ; c1ccc(-c2ccccc2-c2ccccc2-c2ccccc2N(c2ccc3c(c2)-c2ccccc2C32c3ccccc3-c3ccccc32)c2cccc3c2sc2ccccc23)cc1]

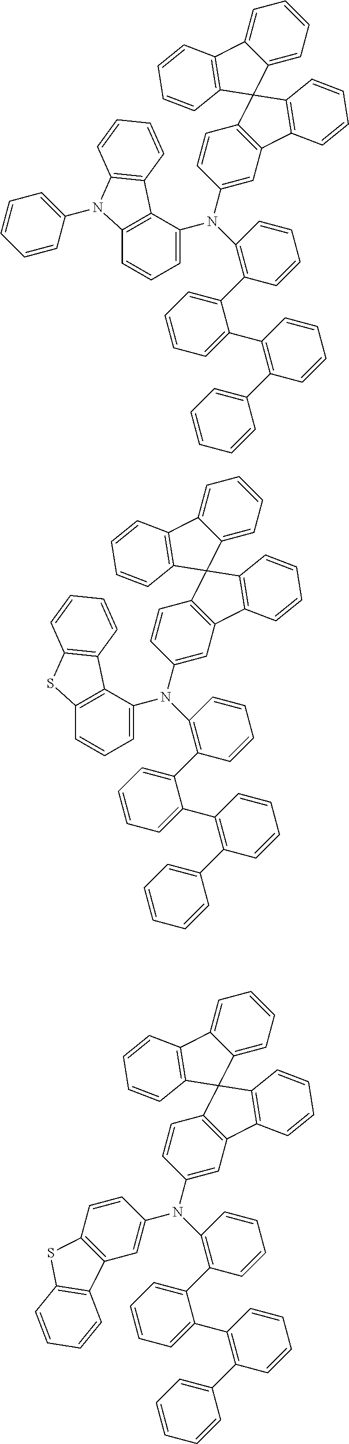

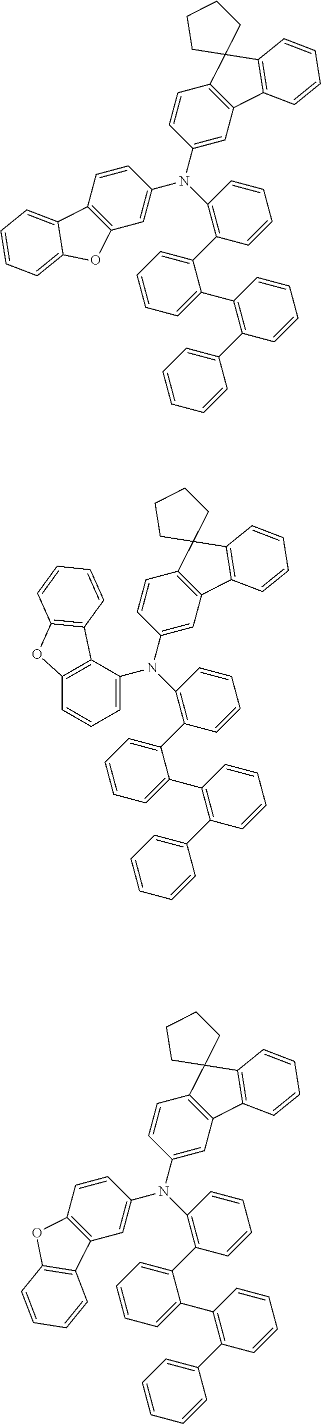 [SMILES: c1ccc(-c2ccccc2-c2ccccc2-c2ccccc2N(c2ccc3c(c2)-c2ccccc2C32CCCC2)c2ccc3c(c2)oc2ccccc23)cc1 ; c1ccc(-c2ccccc2-c2ccccc2-c2ccccc2N(c2ccc3c(c2)-c2ccccc2C32CCCC2)c2cccc3oc4ccccc4c23)cc1 ; c1ccc(-c2ccccc2-c2ccccc2-c2ccccc2N(c2ccc3c(c2)-c2ccccc2C32CCCC2)c2ccc3oc4ccccc4c3c2)cc1]

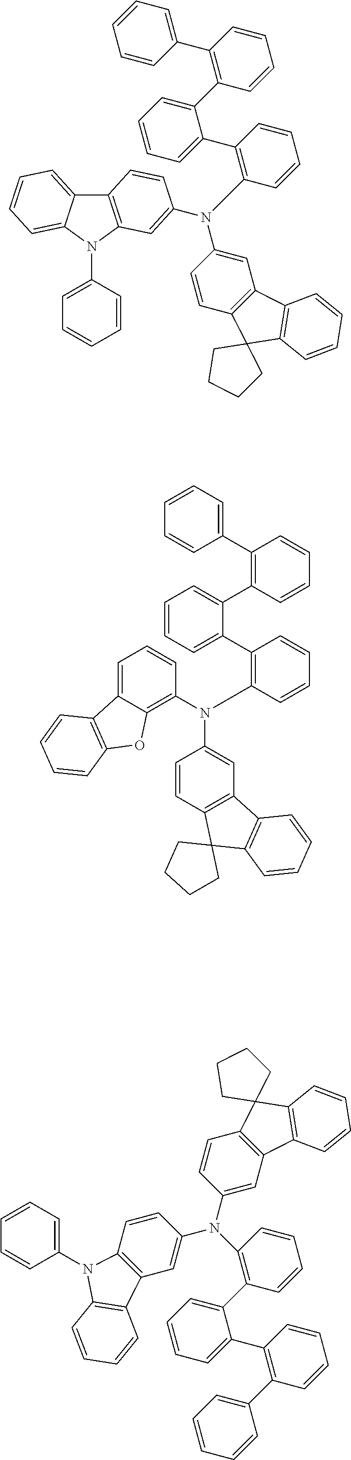 [SMILES: c1ccc(-c2ccccc2-c2ccccc2-c2ccccc2N(c2ccc3c(c2)-c2ccccc2C32CCCC2)c2ccc3c4ccccc4n(-c4ccccc4)c3c2)cc1 ; c1ccc(-c2ccccc2-c2ccccc2-c2ccccc2N(c2ccc3c(c2)-c2ccccc2C32CCCC2)c2cccc3c2oc2ccccc23)cc1 ; c1ccc(-c2ccccc2-c2ccccc2-c2ccccc2N(c2ccc3c(c2)-c2ccccc2C32CCCC2)c2ccc3c(c2)c2ccccc2n3-c2ccccc2)cc1]

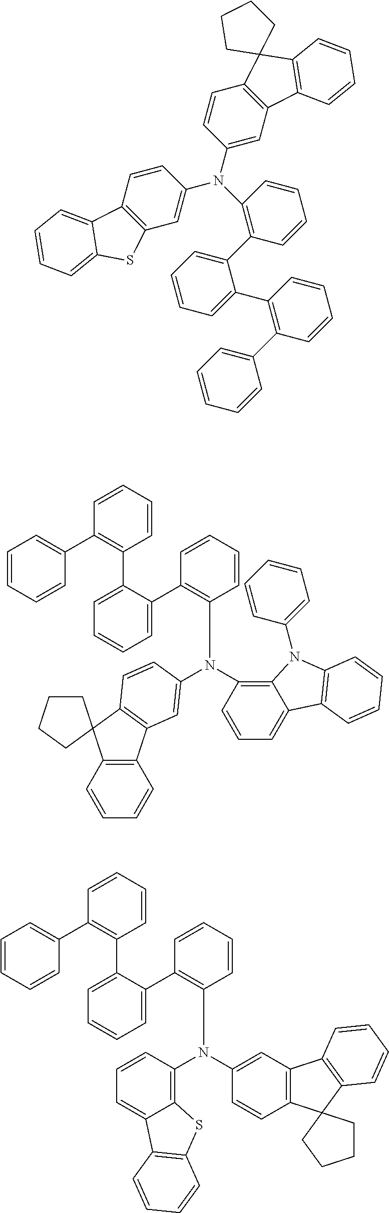 [SMILES: c1ccc(-c2ccccc2-c2ccccc2-c2ccccc2N(c2ccc3c(c2)-c2ccccc2C32CCCC2)c2ccc3c(c2)sc2ccccc23)cc1 ; c1ccc(-c2ccccc2-c2ccccc2-c2ccccc2N(c2ccc3c(c2)-c2ccccc2C32CCCC2)c2cccc3c4ccccc4n(-c4ccccc4)c23)cc1 ; c1ccc(-c2ccccc2-c2ccccc2-c2ccccc2N(c2ccc3c(c2)-c2ccccc2C32CCCC2)c2cccc3c2sc2ccccc23)cc1]

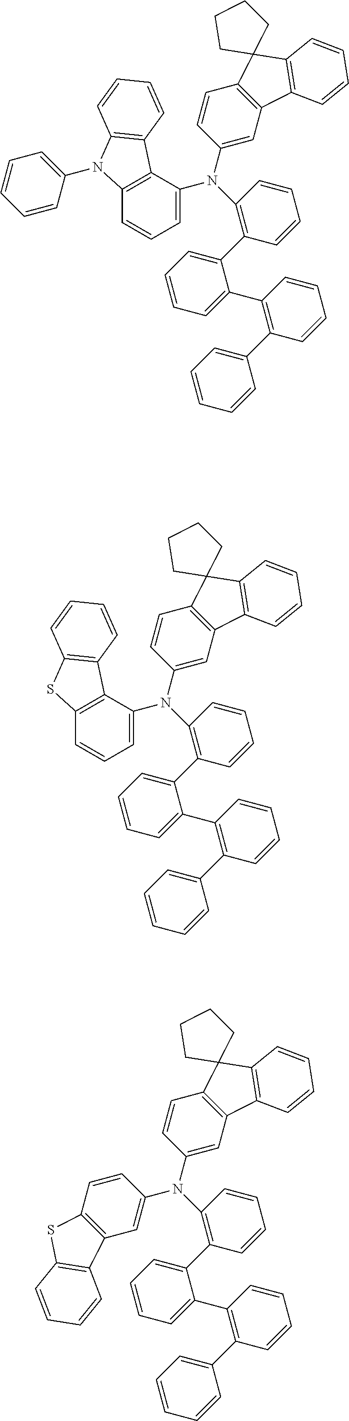 [SMILES: c1ccc(-c2ccccc2-c2ccccc2-c2ccccc2N(c2ccc3c(c2)-c2ccccc2C32CCCC2)c2cccc3c2c2ccccc2n3-c2ccccc2)cc1 ; c1ccc(-c2ccccc2-c2ccccc2-c2ccccc2N(c2ccc3c(c2)-c2ccccc2C32CCCC2)c2cccc3sc4ccccc4c23)cc1 ; c1ccc(-c2ccccc2-c2ccccc2-c2ccccc2N(c2ccc3c(c2)-c2ccccc2C32CCCC2)c2ccc3sc4ccccc4c3c2)cc1]

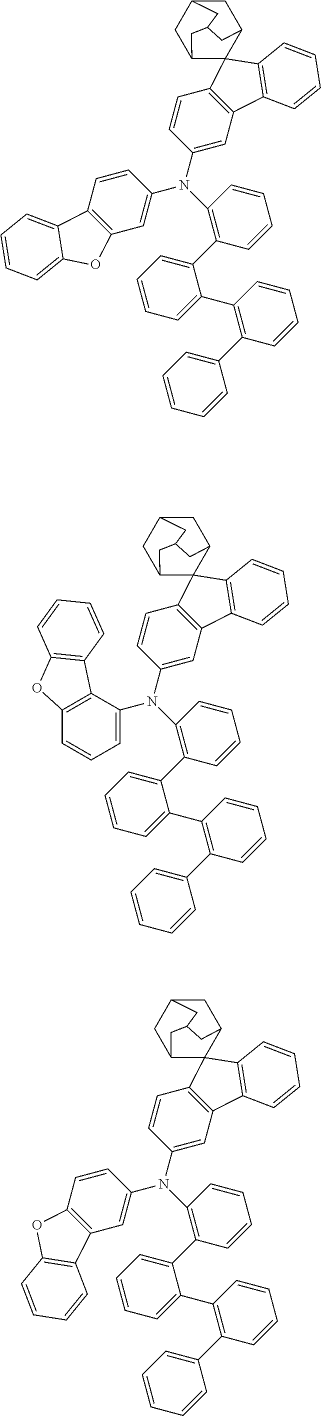 [SMILES: c1ccc(-c2ccccc2-c2ccccc2-c2ccccc2N(c2ccc3c(c2)-c2ccccc2C32C3CC4CC(C3)CC2C4)c2ccc3c(c2)oc2ccccc23)cc1 ; c1ccc(-c2ccccc2-c2ccccc2-c2ccccc2N(c2ccc3c(c2)-c2ccccc2C32C3CC4CC(C3)CC2C4)c2cccc3oc4ccccc4c23)cc1 ; c1ccc(-c2ccccc2-c2ccccc2-c2ccccc2N(c2ccc3c(c2)-c2ccccc2C32C3CC4CC(C3)CC2C4)c2ccc3oc4ccccc4c3c2)cc1]

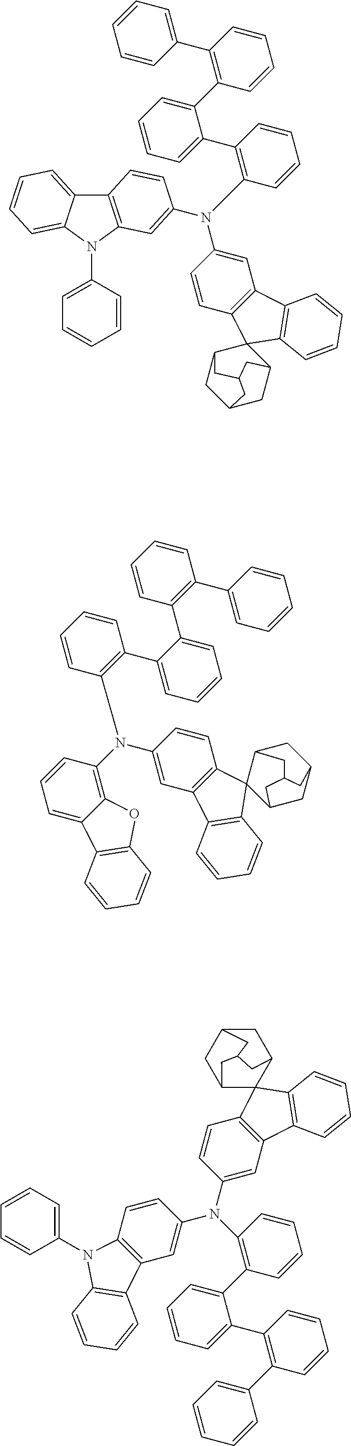 [SMILES: c1ccc(-c2ccccc2-c2ccccc2-c2ccccc2N(c2ccc3c(c2)-c2ccccc2C32C3CC4CC(C3)CC2C4)c2ccc3c4ccccc4n(-c4ccccc4)c3c2)cc1 ; c1ccc(-c2ccccc2-c2ccccc2-c2ccccc2N(c2ccc3c(c2)-c2ccccc2C32C3CC4CC(C3)CC2C4)c2cccc3c2oc2ccccc23)cc1 ; c1ccc(-c2ccccc2-c2ccccc2-c2ccccc2N(c2ccc3c(c2)-c2ccccc2C32C3CC4CC(C3)CC2C4)c2ccc3c(c2)c2ccccc2n3-c2ccccc2)cc1]

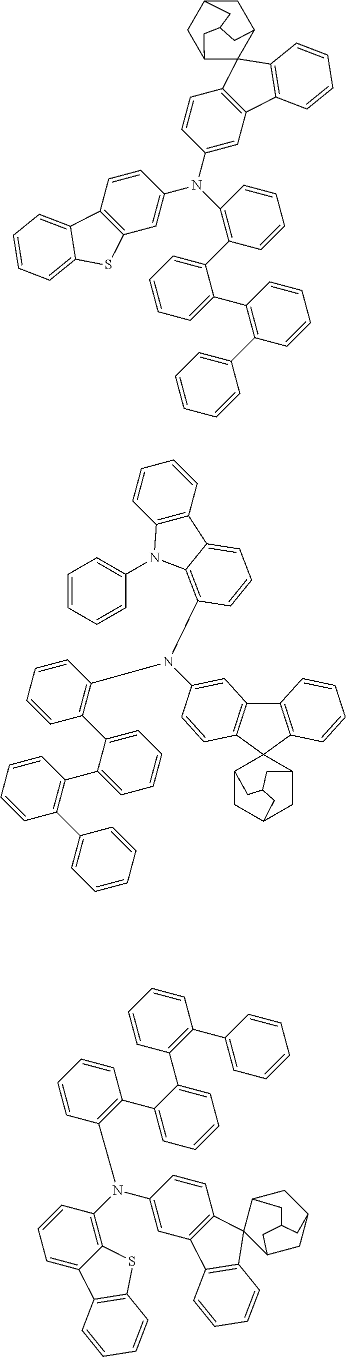 [SMILES: c1ccc(-c2ccccc2-c2ccccc2-c2ccccc2N(c2ccc3c(c2)-c2ccccc2C32C3CC4CC(C3)CC2C4)c2ccc3c(c2)sc2ccccc23)cc1 ; c1ccc(-c2ccccc2-c2ccccc2-c2ccccc2N(c2ccc3c(c2)-c2ccccc2C32C3CC4CC(C3)CC2C4)c2cccc3c4ccccc4n(-c4ccccc4)c23)cc1 ; c1ccc(-c2ccccc2-c2ccccc2-c2ccccc2N(c2ccc3c(c2)-c2ccccc2C32C3CC4CC(C3)CC2C4)c2cccc3c2sc2ccccc23)cc1]

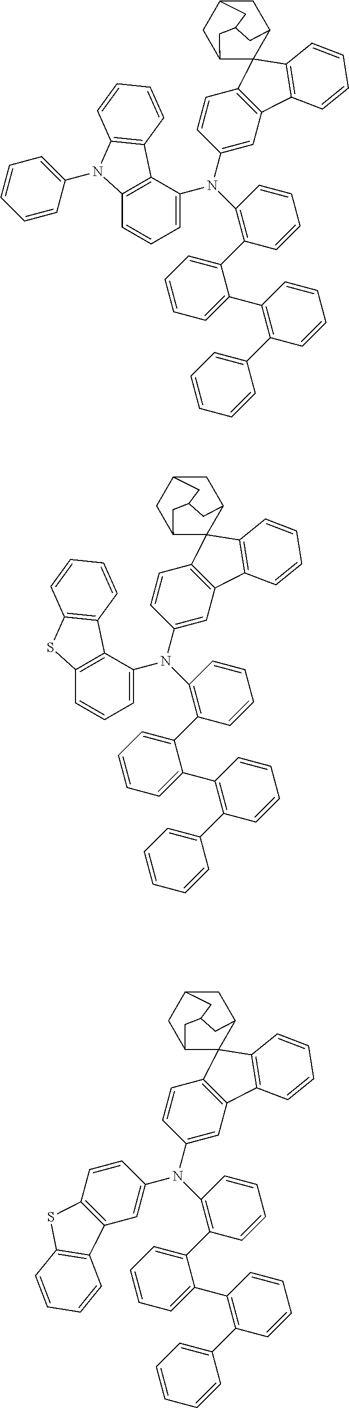 [SMILES: c1ccc(-c2ccccc2-c2ccccc2-c2ccccc2N(c2ccc3c(c2)-c2ccccc2C32C3CC4CC(C3)CC2C4)c2cccc3c2c2ccccc2n3-c2ccccc2)cc1 ; c1ccc(-c2ccccc2-c2ccccc2-c2ccccc2N(c2ccc3c(c2)-c2ccccc2C32C3CC4CC(C3)CC2C4)c2cccc3sc4ccccc4c23)cc1 ; c1ccc(-c2ccccc2-c2ccccc2-c2ccccc2N(c2ccc3c(c2)-c2ccccc2C32C3CC4CC(C3)CC2C4)c2ccc3sc4ccccc4c3c2)cc1]

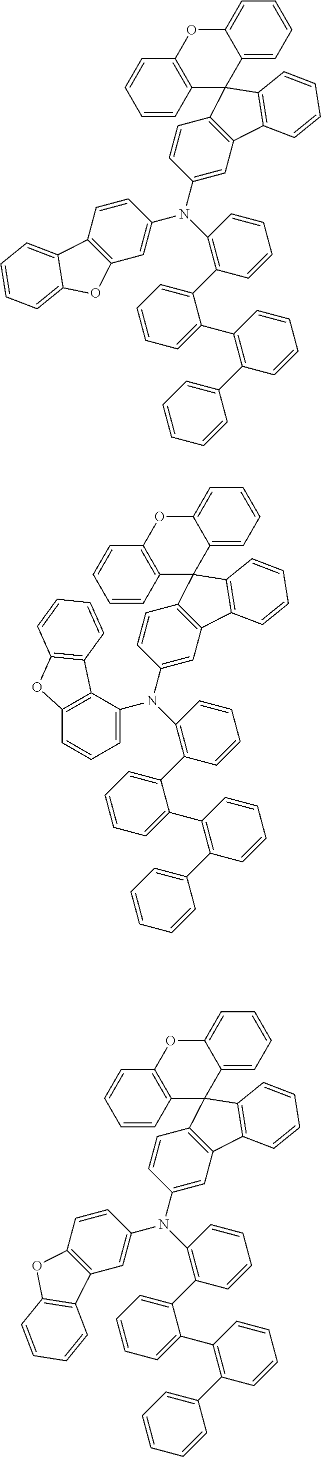

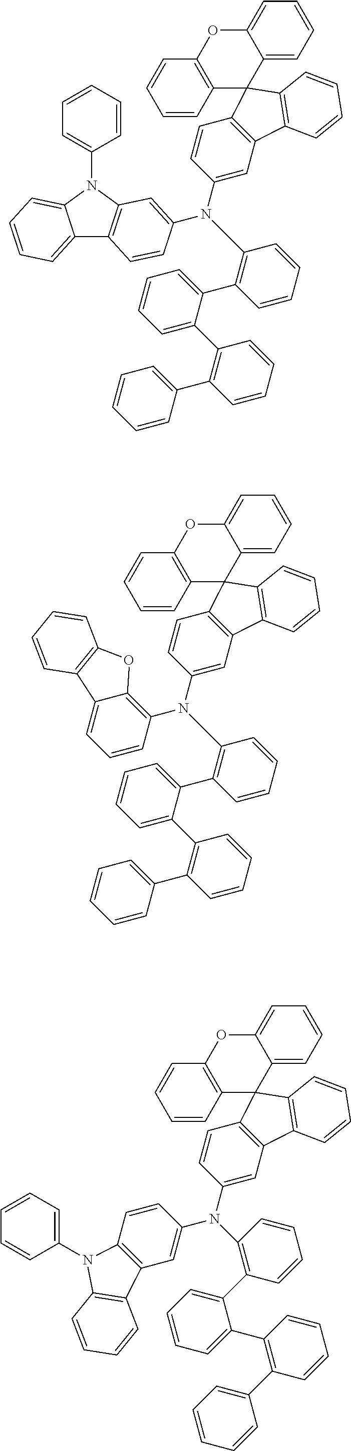 [SMILES: c1ccc(-c2ccccc2-c2ccccc2-c2ccccc2N(c2ccc3c(c2)-c2ccccc2C32c3ccccc3Oc3ccccc32)c2ccc3c4ccccc4n(-c4ccccc4)c3c2)cc1 ; c1ccc(-c2ccccc2-c2ccccc2-c2ccccc2N(c2ccc3c(c2)-c2ccccc2C32c3ccccc3Oc3ccccc32)c2cccc3c2oc2ccccc23)cc1 ; c1ccc(-c2ccccc2-c2ccccc2-c2ccccc2N(c2ccc3c(c2)-c2ccccc2C32c3ccccc3Oc3ccccc32)c2ccc3c(c2)c2ccccc2n3-c2ccccc2)cc1]

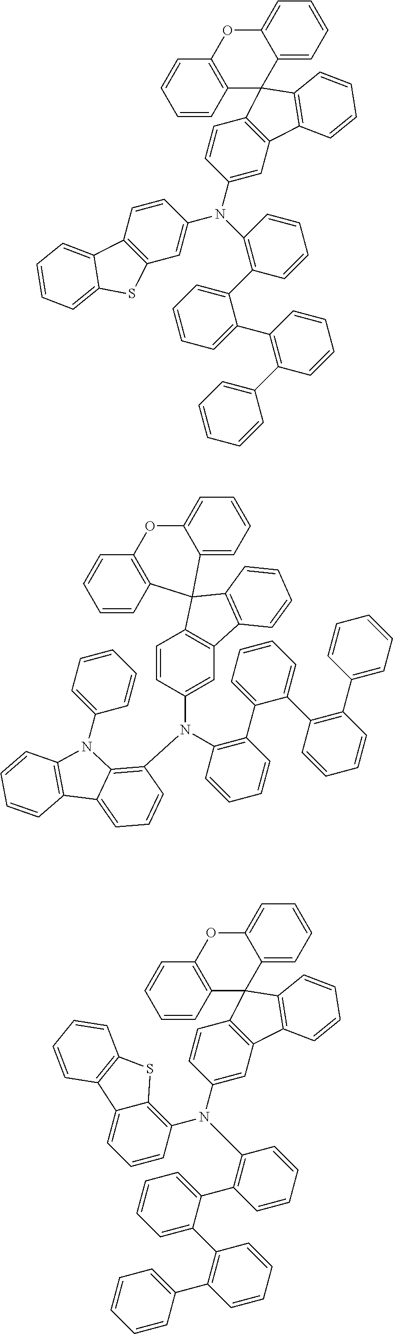 [SMILES: c1ccc(-c2ccccc2-c2ccccc2-c2ccccc2N(c2ccc3c(c2)-c2ccccc2C32c3ccccc3Oc3ccccc32)c2ccc3c(c2)sc2ccccc23)cc1 ; c1ccc(-c2ccccc2-c2ccccc2-c2ccccc2N(c2ccc3c(c2)-c2ccccc2C32c3ccccc3Oc3ccccc32)c2cccc3c4ccccc4n(-c4ccccc4)c23)cc1 ; c1ccc(-c2ccccc2-c2ccccc2-c2ccccc2N(c2ccc3c(c2)-c2ccccc2C32c3ccccc3Oc3ccccc32)c2cccc3c2sc2ccccc23)cc1]

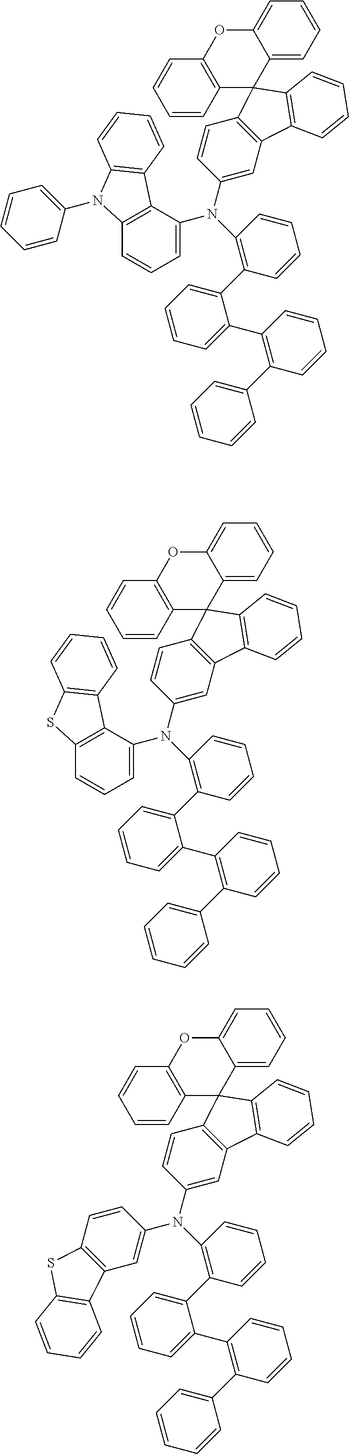 [SMILES: c1ccc(-c2ccccc2-c2ccccc2-c2ccccc2N(c2ccc3c(c2)-c2ccccc2C32c3ccccc3Oc3ccccc32)c2cccc3c2c2ccccc2n3-c2ccccc2)cc1 ; c1ccc(-c2ccccc2-c2ccccc2-c2ccccc2N(c2ccc3c(c2)-c2ccccc2C32c3ccccc3Oc3ccccc32)c2cccc3sc4ccccc4c23)cc1 ; c1ccc(-c2ccccc2-c2ccccc2-c2ccccc2N(c2ccc3c(c2)-c2ccccc2C32c3ccccc3Oc3ccccc32)c2ccc3sc4ccccc4c3c2)cc1]

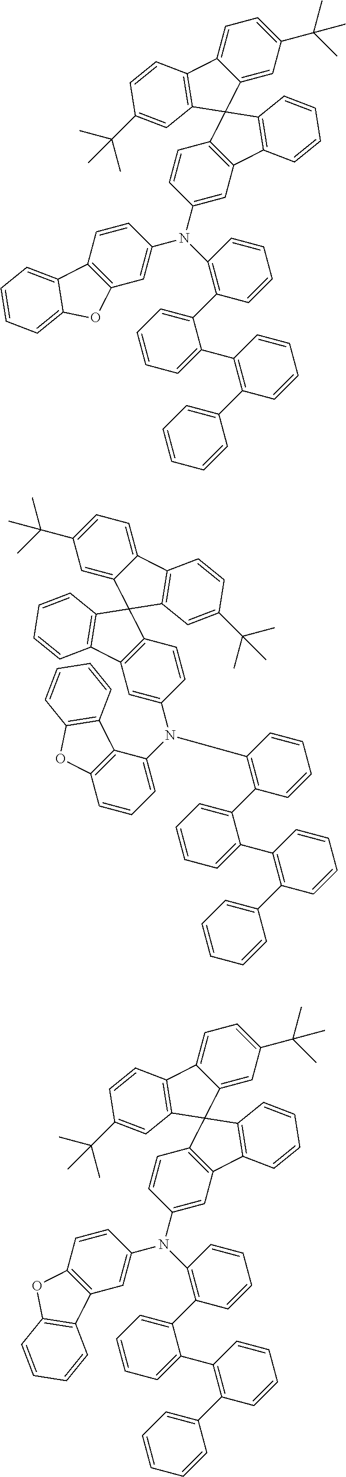

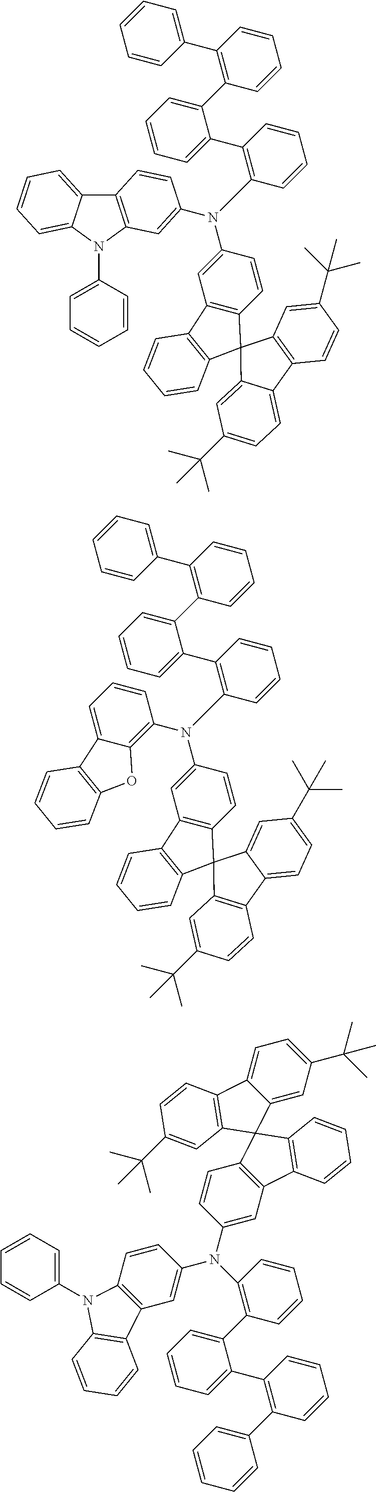 [SMILES: CC(C)(C)c1ccc2c(c1)C1(c3ccccc3-c3cc(N(c4ccc5c6ccccc6n(-c6ccccc6)c5c4)c4ccccc4-c4ccccc4-c4ccccc4-c4ccccc4)ccc31)c1cc(C(C)(C)C)ccc1-2 ; CC(C)(C)c1ccc2c(c1)C1(c3ccccc3-c3cc(N(c4ccccc4-c4ccccc4-c4ccccc4-c4ccccc4)c4cccc5c4oc4ccccc45)ccc31)c1cc(C(C)(C)C)ccc1-2 ; CC(C)(C)c1ccc2c(c1)C1(c3ccccc3-c3cc(N(c4ccc5c(c4)c4ccccc4n5-c4ccccc4)c4ccccc4-c4ccccc4-c4ccccc4-c4ccccc4)ccc31)c1cc(C(C)(C)C)ccc1-2]

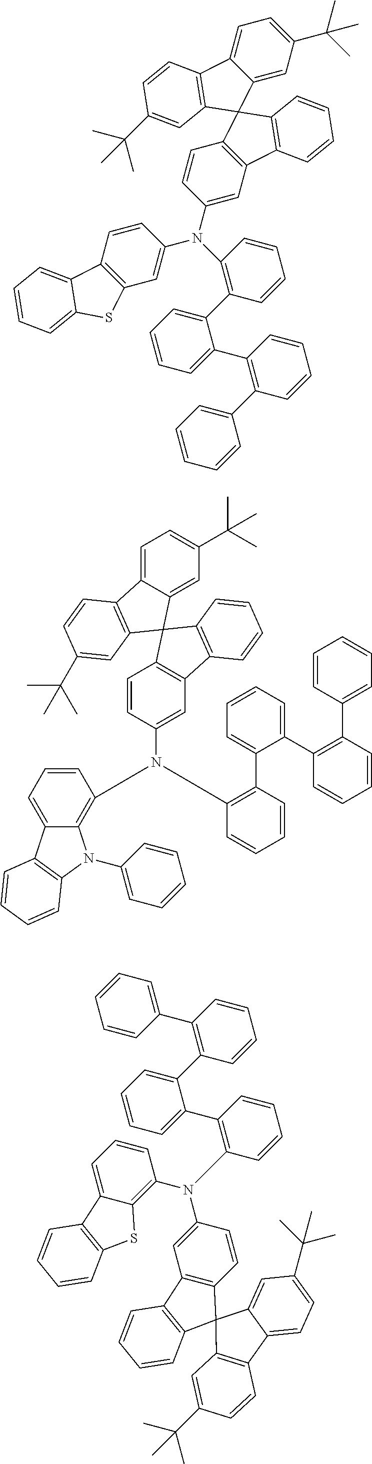

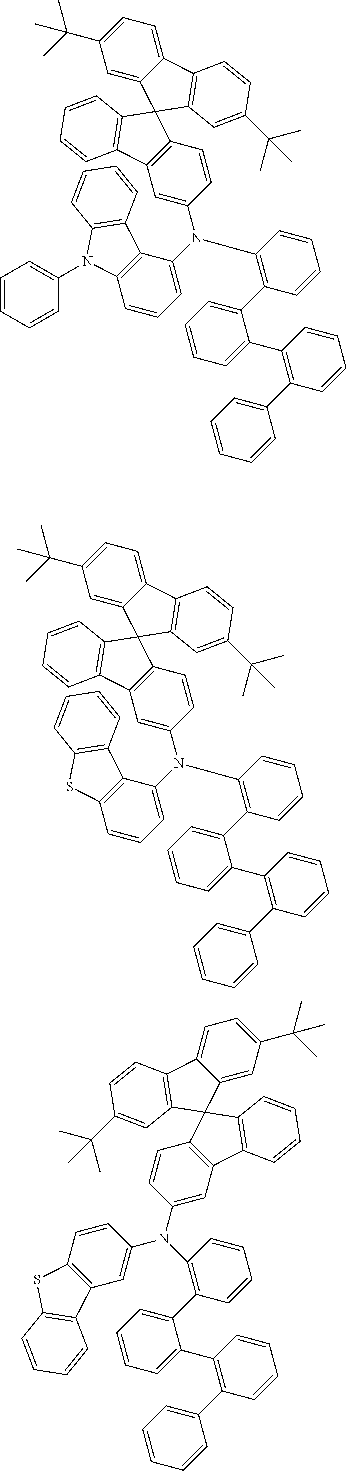

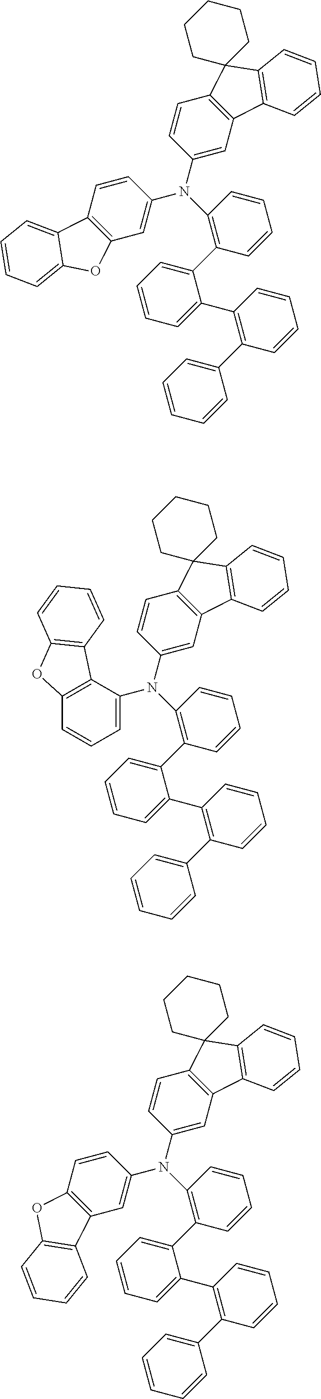 [SMILES: c1ccc(-c2ccccc2-c2ccccc2-c2ccccc2N(c2ccc3c(c2)-c2ccccc2C32CCCCC2)c2ccc3c(c2)oc2ccccc23)cc1 ; c1ccc(-c2ccccc2-c2ccccc2-c2ccccc2N(c2ccc3c(c2)-c2ccccc2C32CCCCC2)c2cccc3oc4ccccc4c23)cc1 ; c1ccc(-c2ccccc2-c2ccccc2-c2ccccc2N(c2ccc3c(c2)-c2ccccc2C32CCCCC2)c2ccc3oc4ccccc4c3c2)cc1]

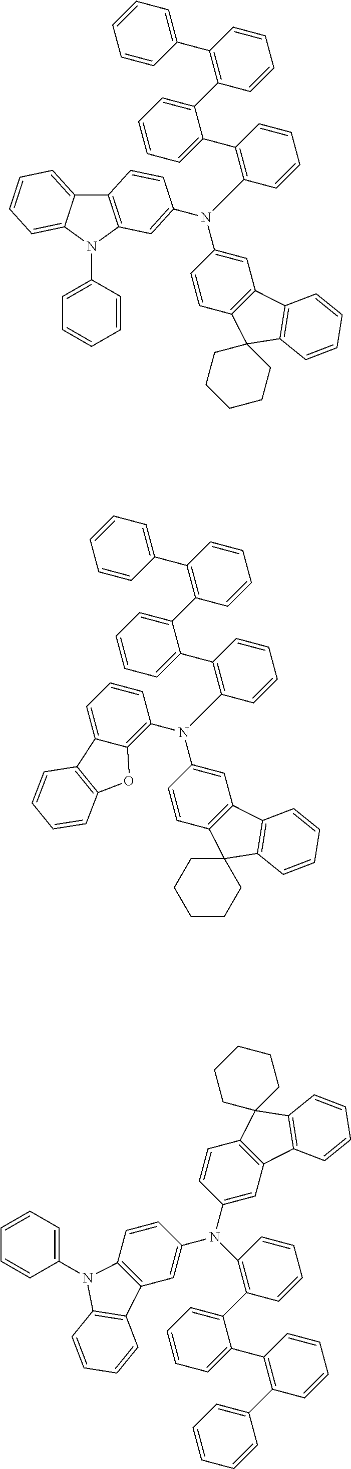 [SMILES: c1ccc(-c2ccccc2-c2ccccc2-c2ccccc2N(c2ccc3c(c2)-c2ccccc2C32CCCCC2)c2ccc3c4ccccc4n(-c4ccccc4)c3c2)cc1 ; c1ccc(-c2ccccc2-c2ccccc2-c2ccccc2N(c2ccc3c(c2)-c2ccccc2C32CCCCC2)c2cccc3c2oc2ccccc23)cc1 ; c1ccc(-c2ccccc2-c2ccccc2-c2ccccc2N(c2ccc3c(c2)-c2ccccc2C32CCCCC2)c2ccc3c(c2)c2ccccc2n3-c2ccccc2)cc1]

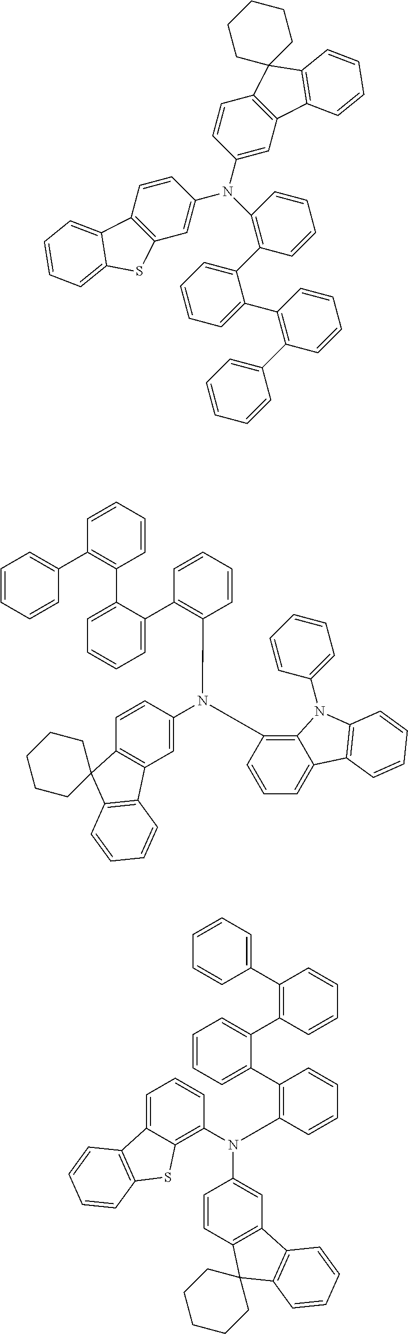 [SMILES: c1ccc(-c2ccccc2-c2ccccc2-c2ccccc2N(c2ccc3c(c2)-c2ccccc2C32CCCCC2)c2ccc3c(c2)sc2ccccc23)cc1 ; c1ccc(-c2ccccc2-c2ccccc2-c2ccccc2N(c2ccc3c(c2)-c2ccccc2C32CCCCC2)c2cccc3c4ccccc4n(-c4ccccc4)c23)cc1 ; c1ccc(-c2ccccc2-c2ccccc2-c2ccccc2N(c2ccc3c(c2)-c2ccccc2C32CCCCC2)c2cccc3c2sc2ccccc23)cc1]

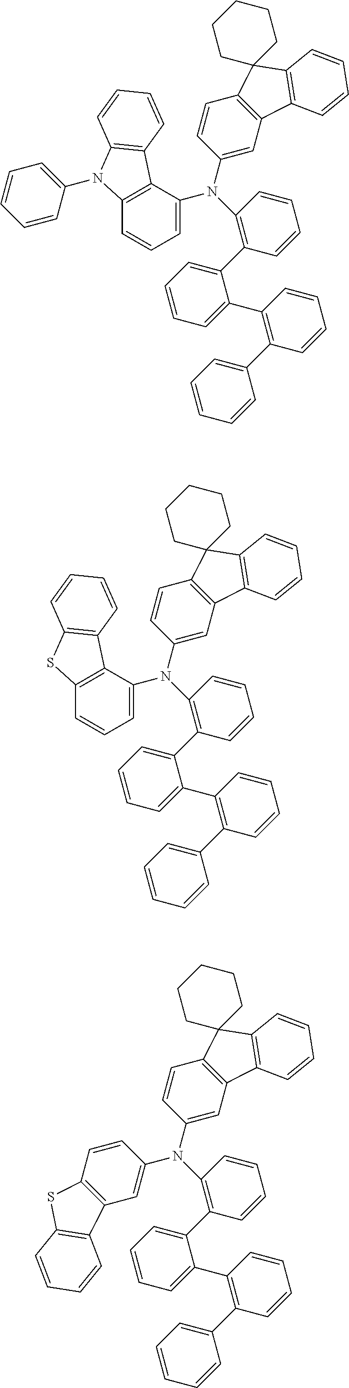 [SMILES: c1ccc(-c2ccccc2-c2ccccc2-c2ccccc2N(c2ccc3c(c2)-c2ccccc2C32CCCCC2)c2cccc3c2c2ccccc2n3-c2ccccc2)cc1 ; c1ccc(-c2ccccc2-c2ccccc2-c2ccccc2N(c2ccc3c(c2)-c2ccccc2C32CCCCC2)c2cccc3sc4ccccc4c23)cc1 ; c1ccc(-c2ccccc2-c2ccccc2-c2ccccc2N(c2ccc3c(c2)-c2ccccc2C32CCCCC2)c2ccc3sc4ccccc4c3c2)cc1]

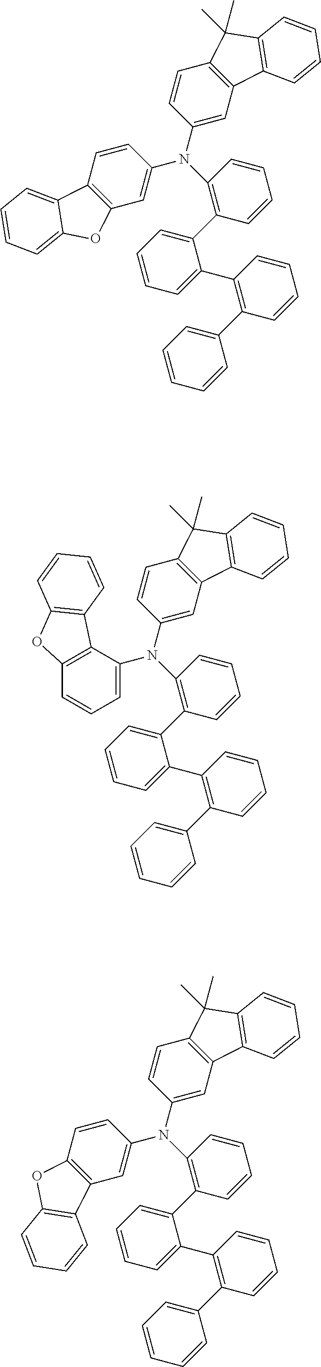 [SMILES: CC1(C)c2ccccc2-c2cc(N(c3ccc4c(c3)oc3ccccc34)c3ccccc3-c3ccccc3-c3ccccc3-c3ccccc3)ccc21 ; CC1(C)c2ccccc2-c2cc(N(c3ccccc3-c3ccccc3-c3ccccc3-c3ccccc3)c3cccc4oc5ccccc5c34)ccc21 ; CC1(C)c2ccccc2-c2cc(N(c3ccc4oc5ccccc5c4c3)c3ccccc3-c3ccccc3-c3ccccc3-c3ccccc3)ccc21]

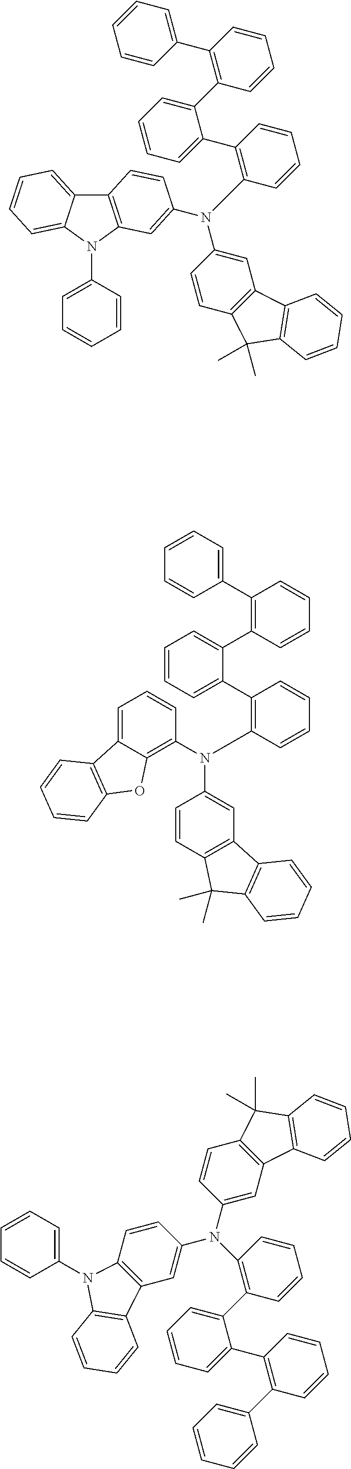 [SMILES: CC1(C)c2ccccc2-c2cc(N(c3ccc4c5ccccc5n(-c5ccccc5)c4c3)c3ccccc3-c3ccccc3-c3ccccc3-c3ccccc3)ccc21 ; CC1(C)c2ccccc2-c2cc(N(c3ccccc3-c3ccccc3-c3ccccc3-c3ccccc3)c3cccc4c3oc3ccccc34)ccc21 ; CC1(C)c2ccccc2-c2cc(N(c3ccc4c(c3)c3ccccc3n4-c3ccccc3)c3ccccc3-c3ccccc3-c3ccccc3-c3ccccc3)ccc21]

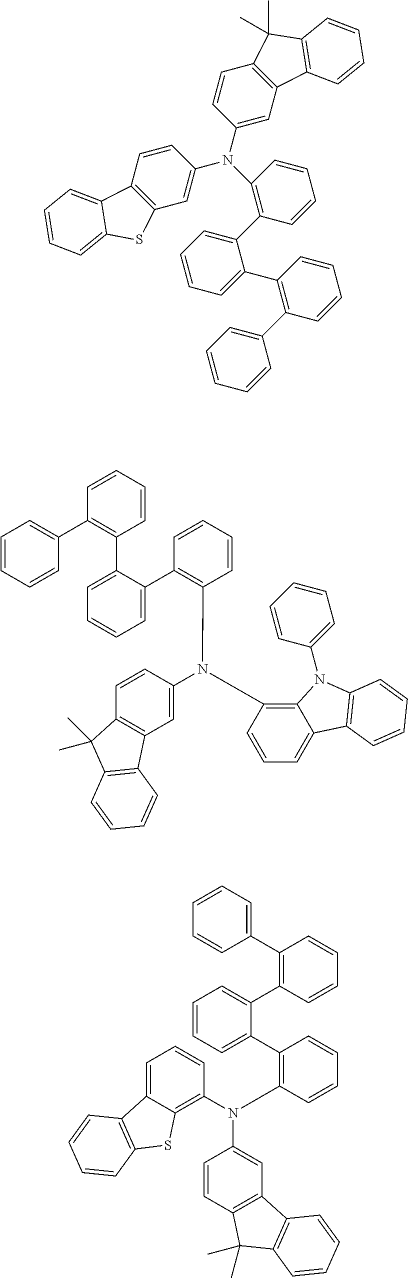 [SMILES: CC1(C)c2ccccc2-c2cc(N(c3ccc4c(c3)sc3ccccc34)c3ccccc3-c3ccccc3-c3ccccc3-c3ccccc3)ccc21 ; CC1(C)c2ccccc2-c2cc(N(c3ccccc3-c3ccccc3-c3ccccc3-c3ccccc3)c3cccc4c5ccccc5n(-c5ccccc5)c34)ccc21 ; CC1(C)c2ccccc2-c2cc(N(c3ccccc3-c3ccccc3-c3ccccc3-c3ccccc3)c3cccc4c3sc3ccccc34)ccc21]

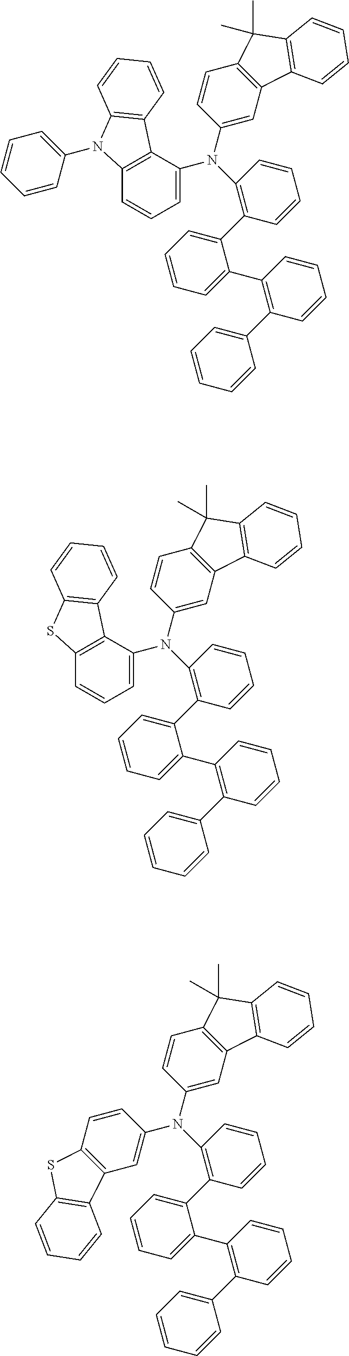 [SMILES: CC1(C)c2ccccc2-c2cc(N(c3ccccc3-c3ccccc3-c3ccccc3-c3ccccc3)c3cccc4c3c3ccccc3n4-c3ccccc3)ccc21 ; CC1(C)c2ccccc2-c2cc(N(c3ccccc3-c3ccccc3-c3ccccc3-c3ccccc3)c3cccc4sc5ccccc5c34)ccc21 ; CC1(C)c2ccccc2-c2cc(N(c3ccc4sc5ccccc5c4c3)c3ccccc3-c3ccccc3-c3ccccc3-c3ccccc3)ccc21]

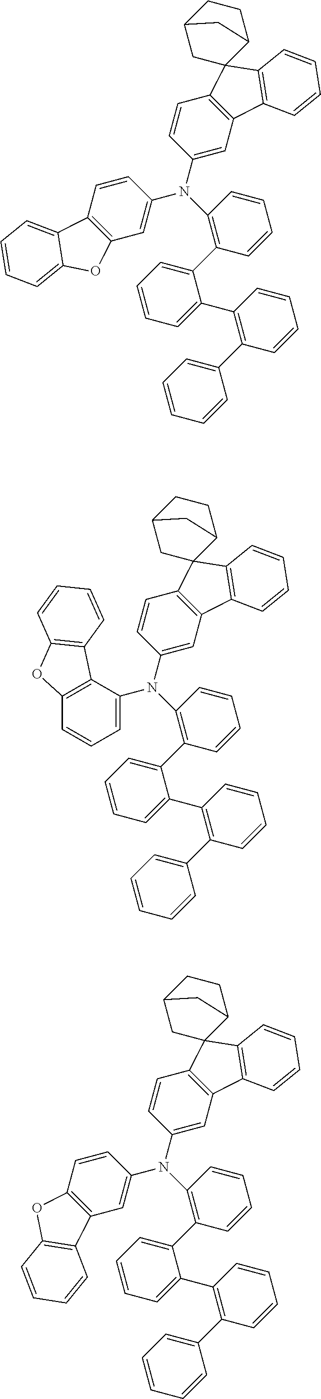 [SMILES: c1ccc(-c2ccccc2-c2ccccc2-c2ccccc2N(c2ccc3c(c2)-c2ccccc2C32CC3CCC2C3)c2ccc3c(c2)oc2ccccc23)cc1 ; c1ccc(-c2ccccc2-c2ccccc2-c2ccccc2N(c2ccc3c(c2)-c2ccccc2C32CC3CCC2C3)c2cccc3oc4ccccc4c23)cc1 ; c1ccc(-c2ccccc2-c2ccccc2-c2ccccc2N(c2ccc3c(c2)-c2ccccc2C32CC3CCC2C3)c2ccc3oc4ccccc4c3c2)cc1]

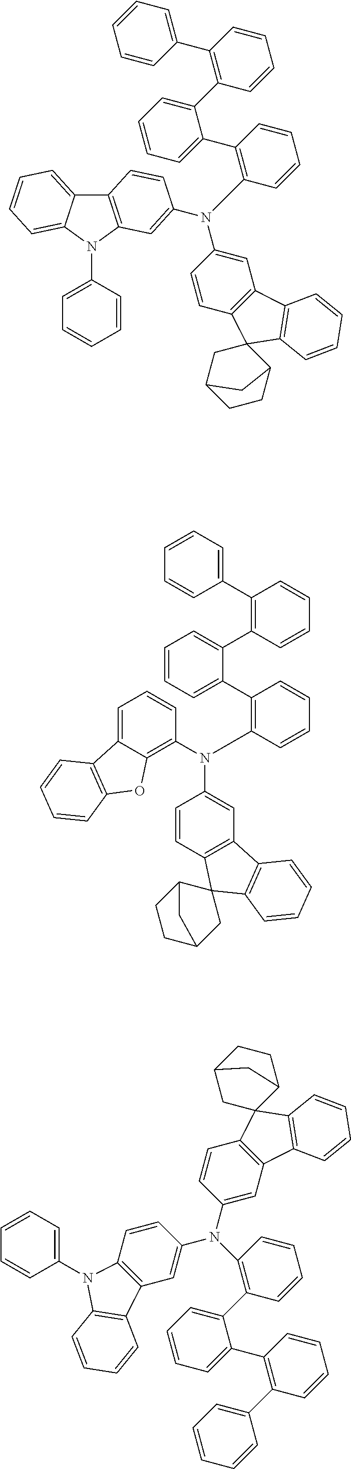 [SMILES: c1ccc(-c2ccccc2-c2ccccc2-c2ccccc2N(c2ccc3c(c2)-c2ccccc2C32CC3CCC2C3)c2ccc3c4ccccc4n(-c4ccccc4)c3c2)cc1 ; c1ccc(-c2ccccc2-c2ccccc2-c2ccccc2N(c2ccc3c(c2)-c2ccccc2C32CC3CCC2C3)c2cccc3c2oc2ccccc23)cc1 ; c1ccc(-c2ccccc2-c2ccccc2-c2ccccc2N(c2ccc3c(c2)-c2ccccc2C32CC3CCC2C3)c2ccc3c(c2)c2ccccc2n3-c2ccccc2)cc1]

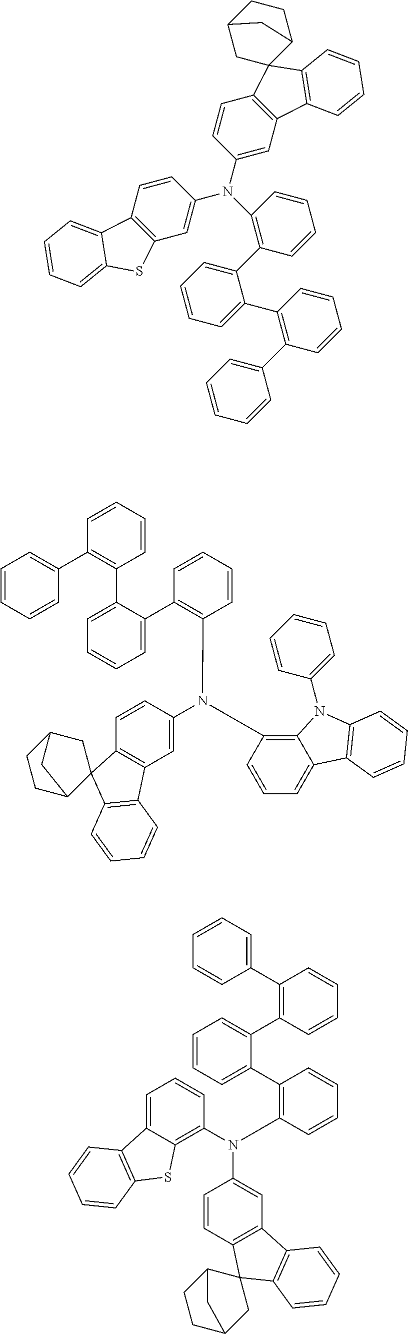 [SMILES: c1ccc(-c2ccccc2-c2ccccc2-c2ccccc2N(c2ccc3c(c2)-c2ccccc2C32CC3CCC2C3)c2ccc3c(c2)sc2ccccc23)cc1 ; c1ccc(-c2ccccc2-c2ccccc2-c2ccccc2N(c2ccc3c(c2)-c2ccccc2C32CC3CCC2C3)c2cccc3c4ccccc4n(-c4ccccc4)c23)cc1 ; c1ccc(-c2ccccc2-c2ccccc2-c2ccccc2N(c2ccc3c(c2)-c2ccccc2C32CC3CCC2C3)c2cccc3c2sc2ccccc23)cc1]

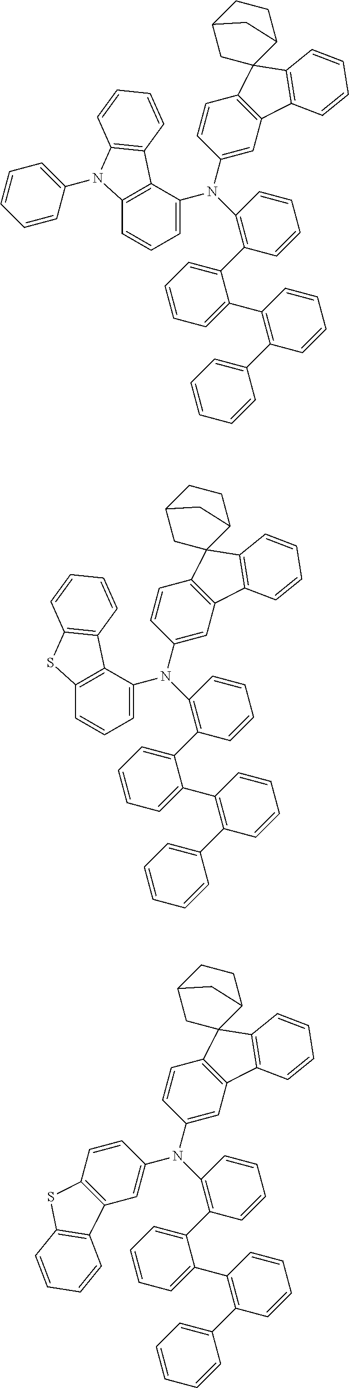 [SMILES: c1ccc(-c2ccccc2-c2ccccc2-c2ccccc2N(c2ccc3c(c2)-c2ccccc2C32CC3CCC2C3)c2cccc3c2c2ccccc2n3-c2ccccc2)cc1 ; c1ccc(-c2ccccc2-c2ccccc2-c2ccccc2N(c2ccc3c(c2)-c2ccccc2C32CC3CCC2C3)c2cccc3sc4ccccc4c23)cc1 ; c1ccc(-c2ccccc2-c2ccccc2-c2ccccc2N(c2ccc3c(c2)-c2ccccc2C32CC3CCC2C3)c2ccc3sc4ccccc4c3c2)cc1]

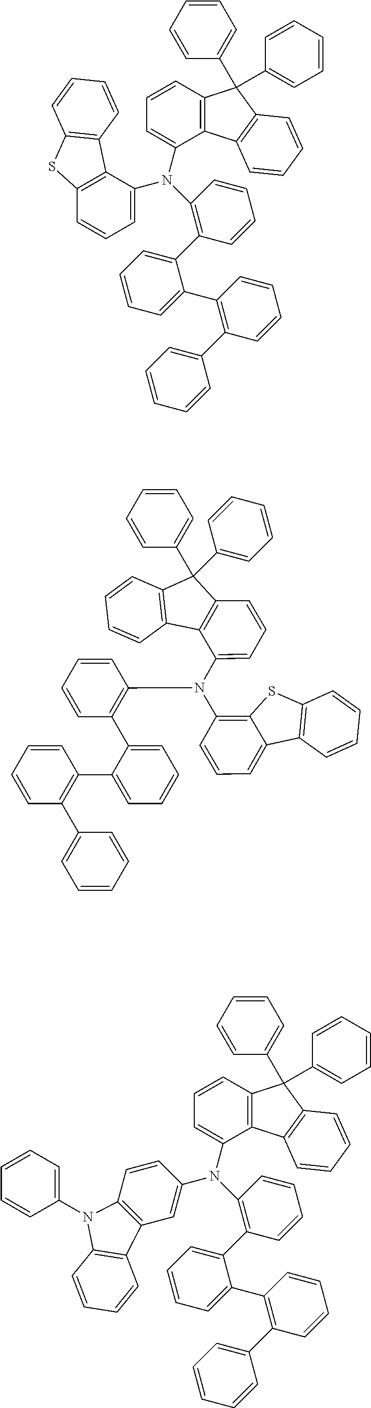 [SMILES: c1ccc(-c2ccccc2-c2ccccc2-c2ccccc2N(c2cccc3c2-c2ccccc2C3(c2ccccc2)c2ccccc2)c2cccc3sc4ccccc4c23)cc1 ; c1ccc(-c2ccccc2-c2ccccc2-c2ccccc2N(c2cccc3c2-c2ccccc2C3(c2ccccc2)c2ccccc2)c2cccc3c2sc2ccccc23)cc1 ; c1ccc(-c2ccccc2-c2ccccc2-c2ccccc2N(c2ccc3c(c2)c2ccccc2n3-c2ccccc2)c2cccc3c2-c2ccccc2C3(c2ccccc2)c2ccccc2)cc1]

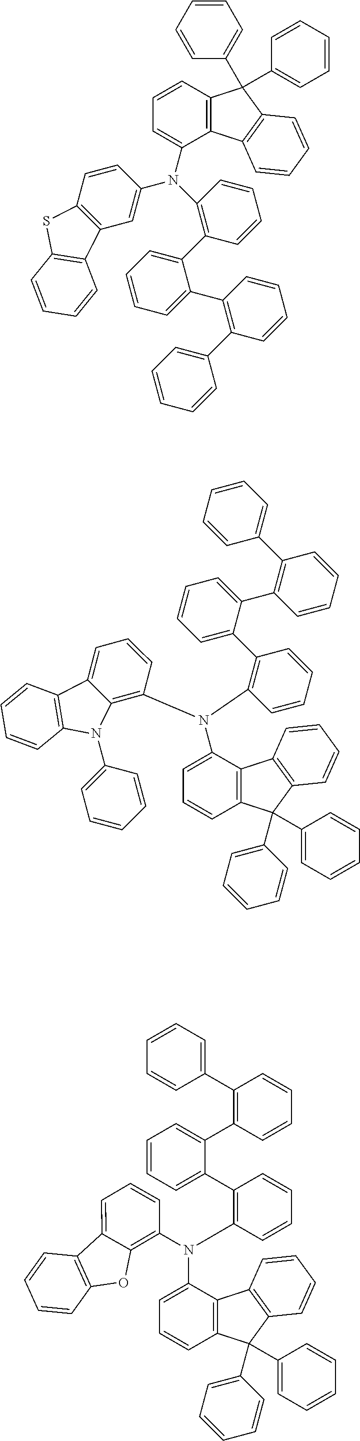 [SMILES: c1ccc(-c2ccccc2-c2ccccc2-c2ccccc2N(c2ccc3sc4ccccc4c3c2)c2cccc3c2-c2ccccc2C3(c2ccccc2)c2ccccc2)cc1 ; c1ccc(-c2ccccc2-c2ccccc2-c2ccccc2N(c2cccc3c2-c2ccccc2C3(c2ccccc2)c2ccccc2)c2cccc3c4ccccc4n(-c4ccccc4)c23)cc1 ; c1ccc(-c2ccccc2-c2ccccc2-c2ccccc2N(c2cccc3c2-c2ccccc2C3(c2ccccc2)c2ccccc2)c2cccc3c2oc2ccccc23)cc1]

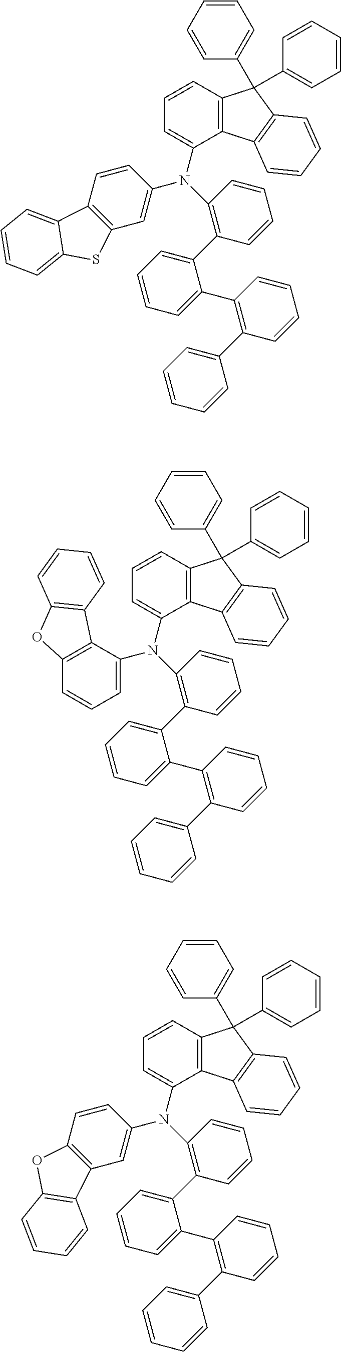 [SMILES: c1ccc(-c2ccccc2-c2ccccc2-c2ccccc2N(c2ccc3c(c2)sc2ccccc23)c2cccc3c2-c2ccccc2C3(c2ccccc2)c2ccccc2)cc1 ; c1ccc(-c2ccccc2-c2ccccc2-c2ccccc2N(c2cccc3c2-c2ccccc2C3(c2ccccc2)c2ccccc2)c2cccc3oc4ccccc4c23)cc1 ; c1ccc(-c2ccccc2-c2ccccc2-c2ccccc2N(c2ccc3oc4ccccc4c3c2)c2cccc3c2-c2ccccc2C3(c2ccccc2)c2ccccc2)cc1]

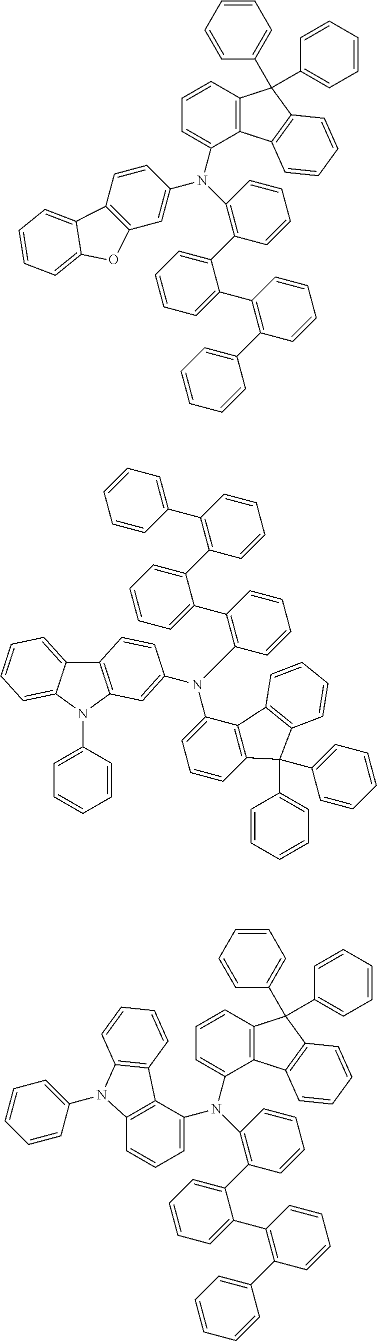 [SMILES: c1ccc(-c2ccccc2-c2ccccc2-c2ccccc2N(c2ccc3c(c2)oc2ccccc23)c2cccc3c2-c2ccccc2C3(c2ccccc2)c2ccccc2)cc1 ; c1ccc(-c2ccccc2-c2ccccc2-c2ccccc2N(c2ccc3c4ccccc4n(-c4ccccc4)c3c2)c2cccc3c2-c2ccccc2C3(c2ccccc2)c2ccccc2)cc1 ; c1ccc(-c2ccccc2-c2ccccc2-c2ccccc2N(c2cccc3c2-c2ccccc2C3(c2ccccc2)c2ccccc2)c2cccc3c2c2ccccc2n3-c2ccccc2)cc1]

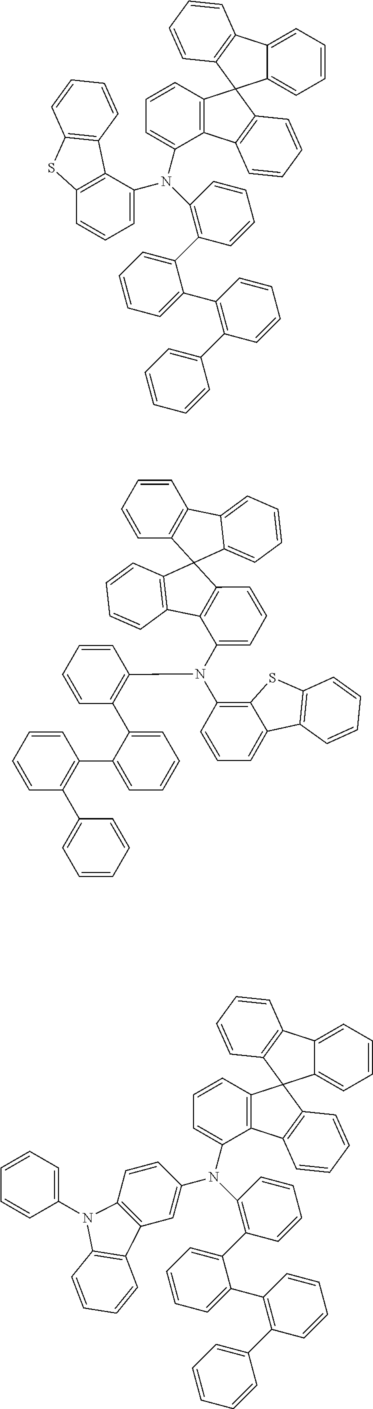 [SMILES: c1ccc(-c2ccccc2-c2ccccc2-c2ccccc2N(c2cccc3c2-c2ccccc2C32c3ccccc3-c3ccccc32)c2cccc3sc4ccccc4c23)cc1 ; c1ccc(-c2ccccc2-c2ccccc2-c2ccccc2N(c2cccc3c2-c2ccccc2C32c3ccccc3-c3ccccc32)c2cccc3c2sc2ccccc23)cc1 ; c1ccc(-c2ccccc2-c2ccccc2-c2ccccc2N(c2ccc3c(c2)c2ccccc2n3-c2ccccc2)c2cccc3c2-c2ccccc2C32c3ccccc3-c3ccccc32)cc1]

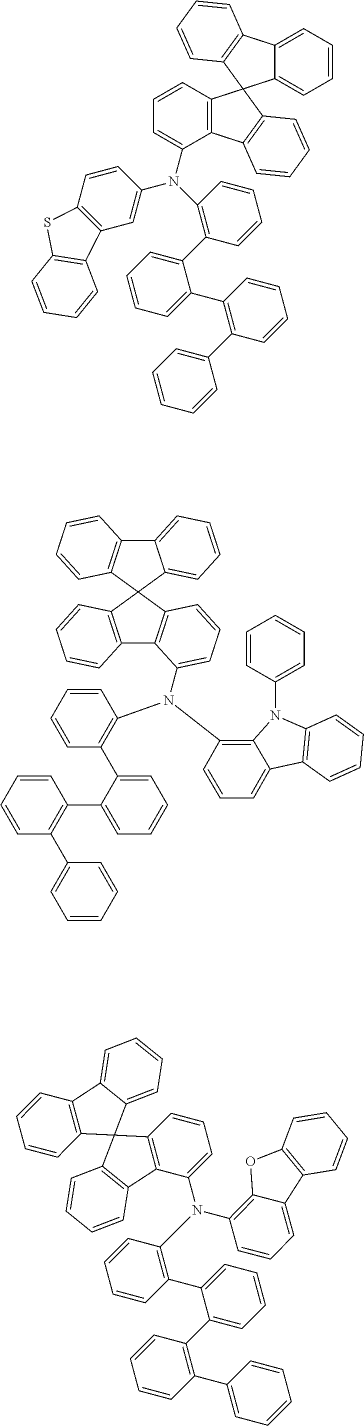 [SMILES: c1ccc(-c2ccccc2-c2ccccc2-c2ccccc2N(c2ccc3sc4ccccc4c3c2)c2cccc3c2-c2ccccc2C32c3ccccc3-c3ccccc32)cc1 ; c1ccc(-c2ccccc2-c2ccccc2-c2ccccc2N(c2cccc3c2-c2ccccc2C32c3ccccc3-c3ccccc32)c2cccc3c4ccccc4n(-c4ccccc4)c23)cc1 ; c1ccc(-c2ccccc2-c2ccccc2-c2ccccc2N(c2cccc3c2-c2ccccc2C32c3ccccc3-c3ccccc32)c2cccc3c2oc2ccccc23)cc1]

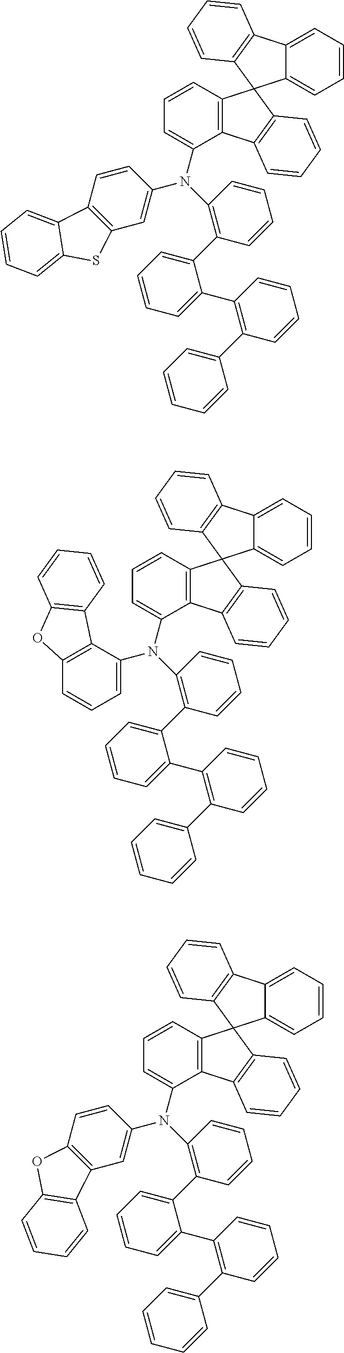 [SMILES: c1ccc(-c2ccccc2-c2ccccc2-c2ccccc2N(c2ccc3c(c2)sc2ccccc23)c2cccc3c2-c2ccccc2C32c3ccccc3-c3ccccc32)cc1 ; c1ccc(-c2ccccc2-c2ccccc2-c2ccccc2N(c2cccc3c2-c2ccccc2C32c3ccccc3-c3ccccc32)c2cccc3oc4ccccc4c23)cc1 ; c1ccc(-c2ccccc2-c2ccccc2-c2ccccc2N(c2ccc3oc4ccccc4c3c2)c2cccc3c2-c2ccccc2C32c3ccccc3-c3ccccc32)cc1]

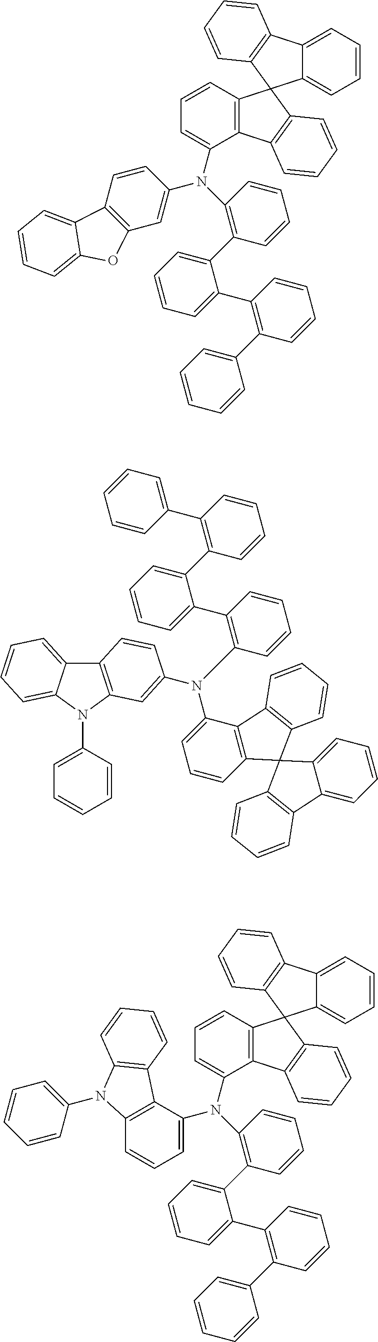 [SMILES: c1ccc(-c2ccccc2-c2ccccc2-c2ccccc2N(c2ccc3c(c2)oc2ccccc23)c2cccc3c2-c2ccccc2C32c3ccccc3-c3ccccc32)cc1 ; c1ccc(-c2ccccc2-c2ccccc2-c2ccccc2N(c2ccc3c4ccccc4n(-c4ccccc4)c3c2)c2cccc3c2-c2ccccc2C32c3ccccc3-c3ccccc32)cc1 ; c1ccc(-c2ccccc2-c2ccccc2-c2ccccc2N(c2cccc3c2-c2ccccc2C32c3ccccc3-c3ccccc32)c2cccc3c2c2ccccc2n3-c2ccccc2)cc1]

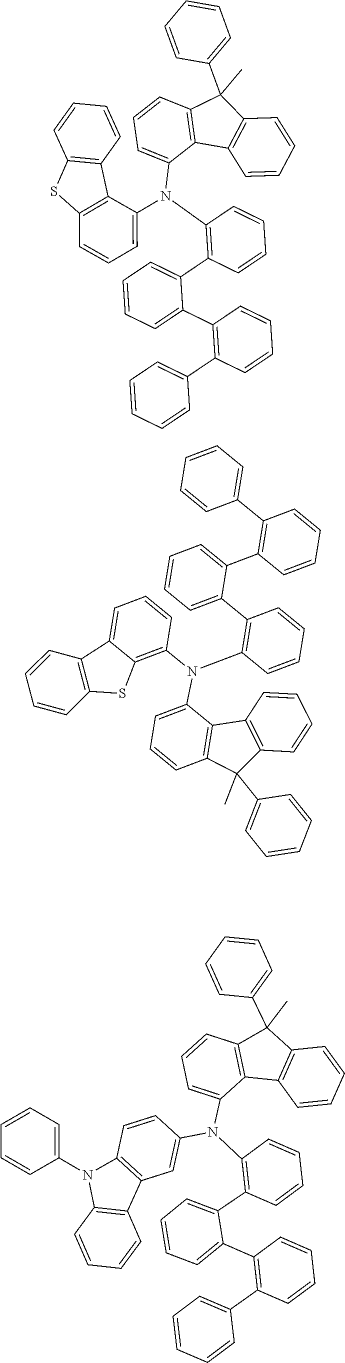

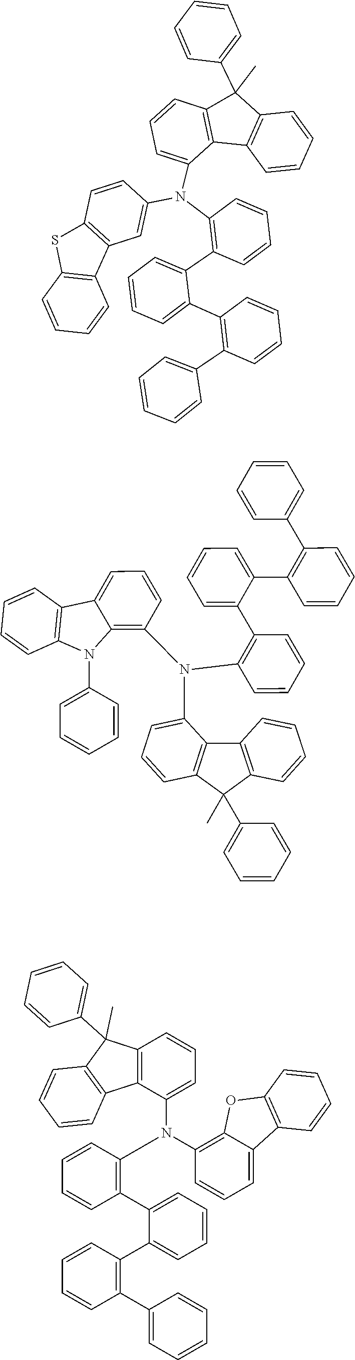 [SMILES: CC1(c2ccccc2)c2ccccc2-c2c(N(c3ccc4sc5ccccc5c4c3)c3ccccc3-c3ccccc3-c3ccccc3-c3ccccc3)cccc21 ; CC1(c2ccccc2)c2ccccc2-c2c(N(c3ccccc3-c3ccccc3-c3ccccc3-c3ccccc3)c3cccc4c5ccccc5n(-c5ccccc5)c34)cccc21 ; CC1(c2ccccc2)c2ccccc2-c2c(N(c3ccccc3-c3ccccc3-c3ccccc3-c3ccccc3)c3cccc4c3oc3ccccc34)cccc21]

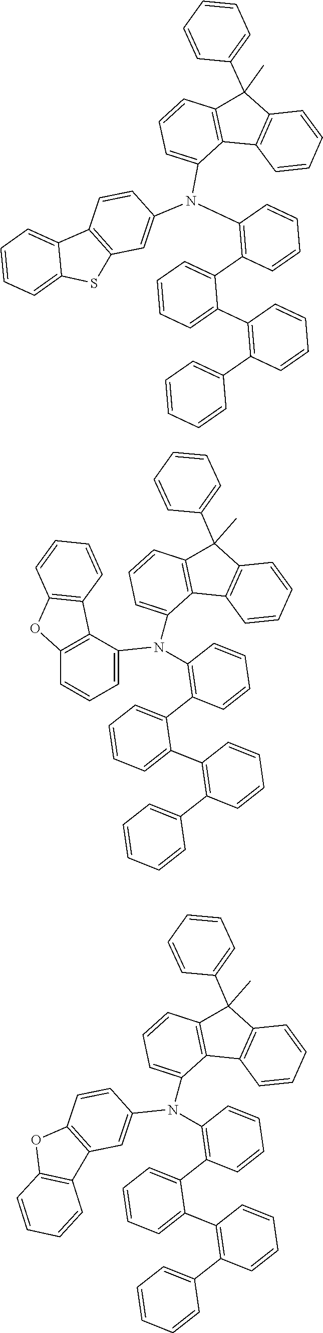

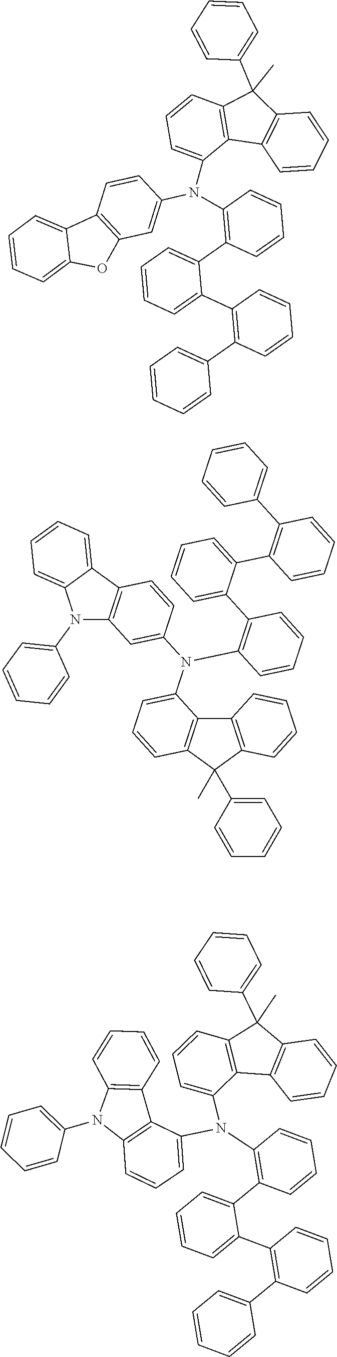

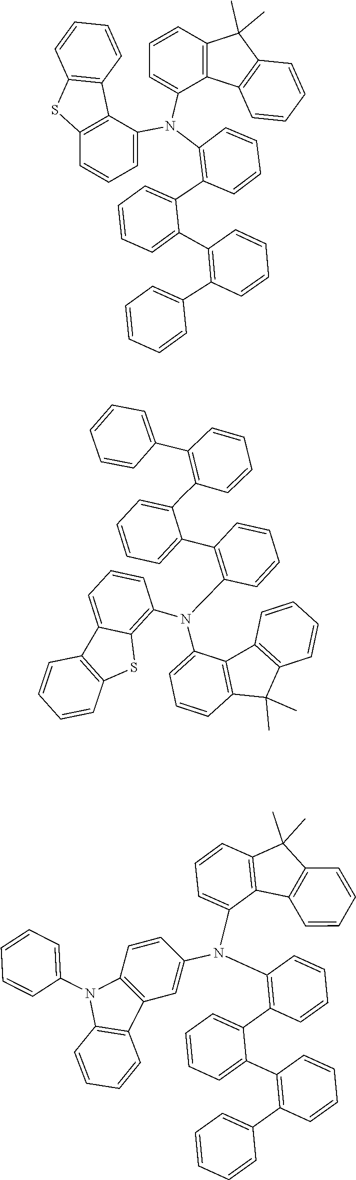 [SMILES: CC1(C)c2ccccc2-c2c(N(c3ccccc3-c3ccccc3-c3ccccc3-c3ccccc3)c3cccc4sc5ccccc5c34)cccc21 ; CC1(C)c2ccccc2-c2c(N(c3ccccc3-c3ccccc3Cc3ccccc3-c3ccccc3)c3cccc4c3sc3ccccc34)cccc21 ; CC1(C)c2ccccc2-c2c(N(c3ccc4c(c3)c3ccccc3n4-c3ccccc3)c3ccccc3-c3ccccc3-c3ccccc3-c3ccccc3)cccc21]

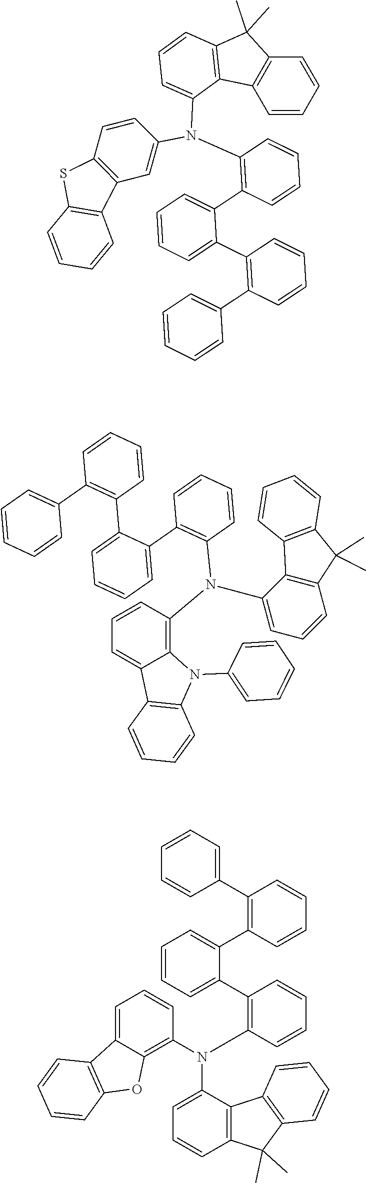 [SMILES: CC1(C)c2ccccc2-c2c(N(c3ccc4sc5ccccc5c4c3)c3ccccc3-c3ccccc3-c3ccccc3-c3ccccc3)cccc21 ; CC1(C)c2ccccc2-c2c(N(c3ccccc3-c3ccccc3-c3ccccc3-c3ccccc3)c3cccc4c5ccccc5n(-c5ccccc5)c34)cccc21 ; CC1(C)c2ccccc2-c2c(N(c3ccccc3-c3ccccc3-c3ccccc3-c3ccccc3)c3cccc4c3oc3ccccc34)cccc21]

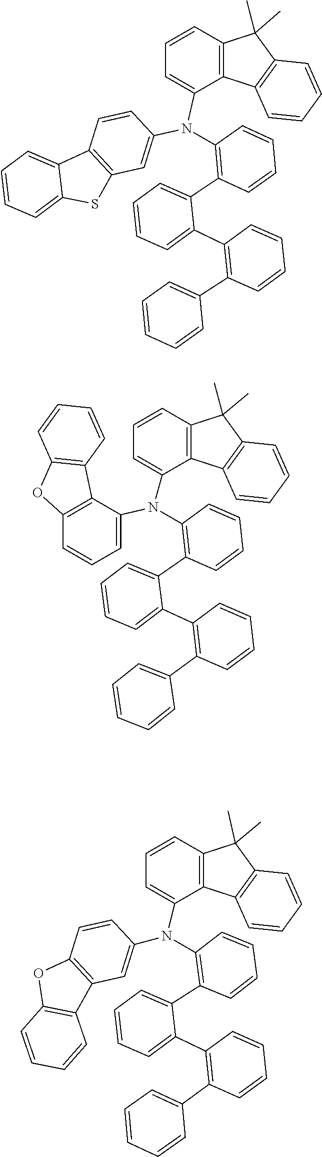 [SMILES: CC1(C)c2ccccc2-c2c(N(c3ccc4c(c3)sc3ccccc34)c3ccccc3-c3ccccc3-c3ccccc3-c3ccccc3)cccc21 ; CC1(C)c2ccccc2-c2c(N(c3ccccc3-c3ccccc3-c3ccccc3-c3ccccc3)c3cccc4oc5ccccc5c34)cccc21 ; CC1(C)c2ccccc2-c2c(N(c3ccc4oc5ccccc5c4c3)c3ccccc3-c3ccccc3-c3ccccc3-c3ccccc3)cccc21]

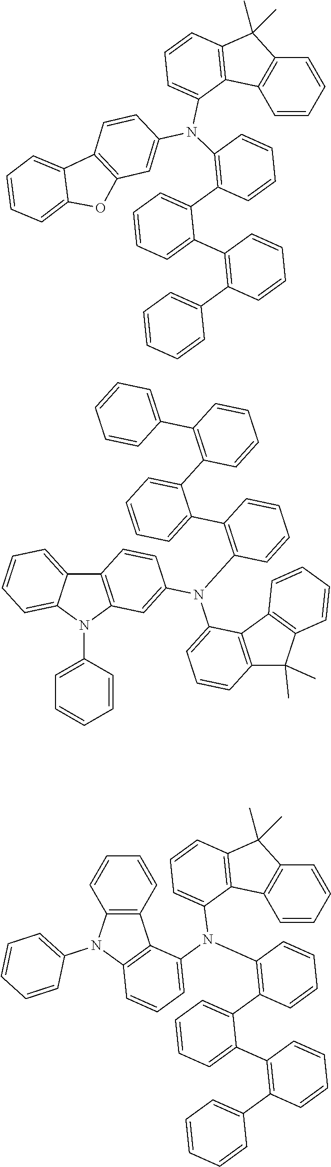

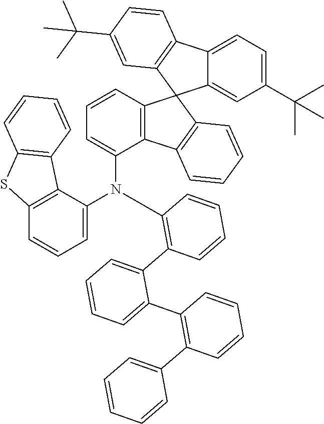 [SMILES: CC(C)(C)c1ccc2c(c1)C1(c3cc(C(C)(C)C)ccc3-2)c2ccccc2-c2c(N(c3ccccc3-c3ccccc3-c3ccccc3-c3ccccc3)c3cccc4sc5ccccc5c34)cccc21]

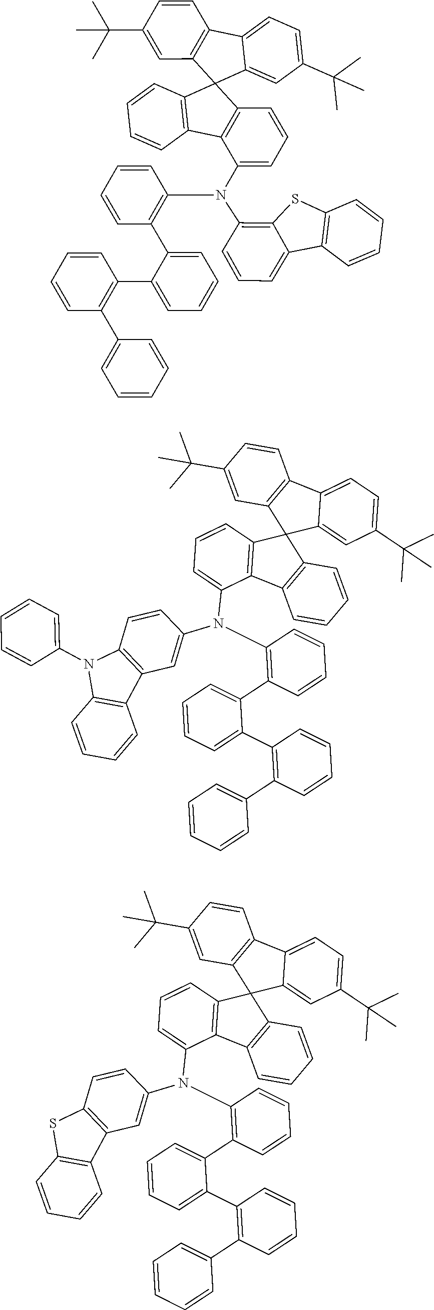

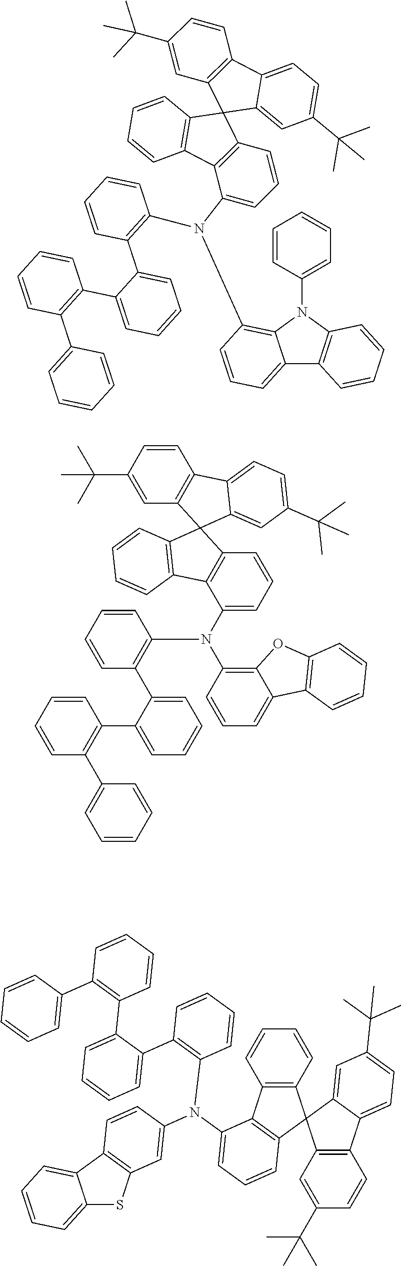

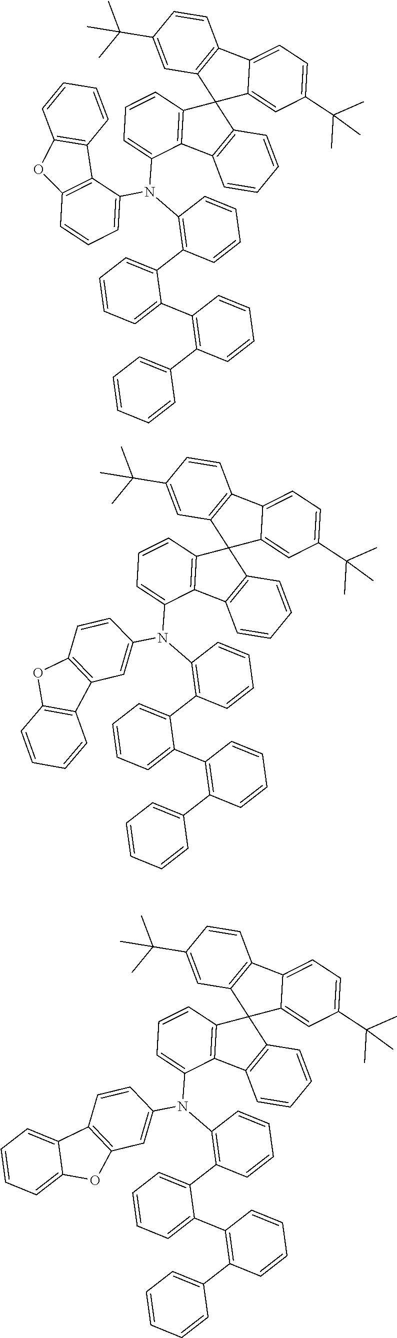

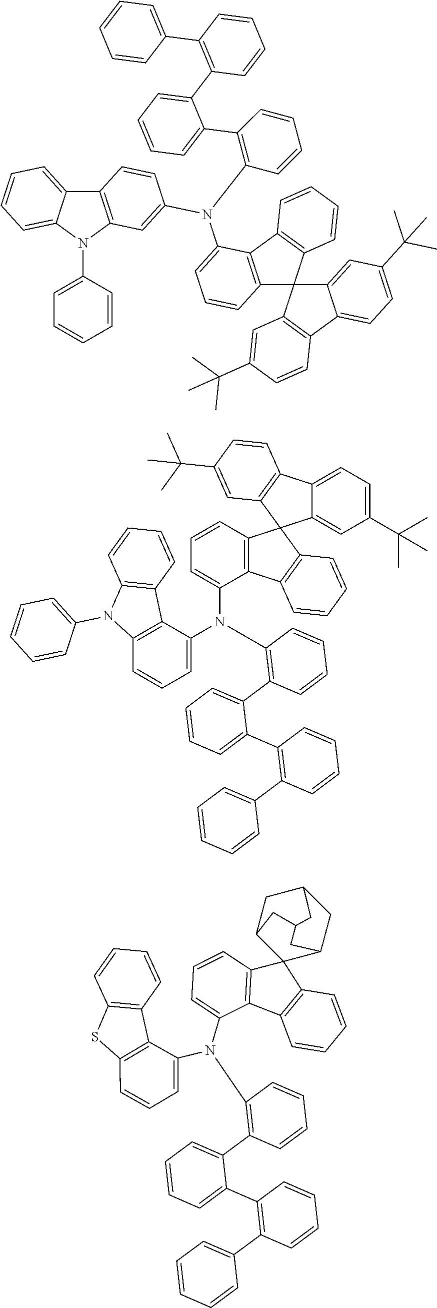

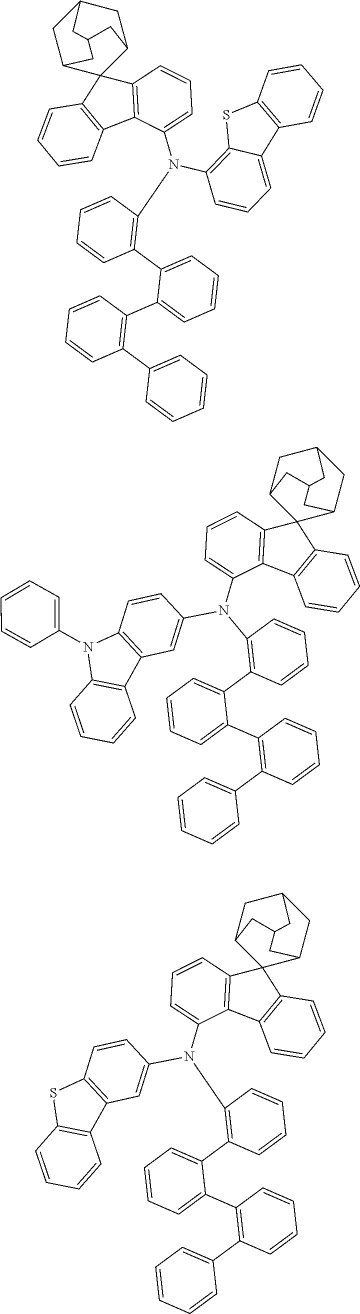 [SMILES: c1ccc(-c2ccccc2-c2ccccc2-c2ccccc2N(c2cccc3c2-c2ccccc2C32C3CC4CC(C3)CC2C4)c2cccc3c2sc2ccccc23)cc1 ; c1ccc(-c2ccccc2-c2ccccc2-c2ccccc2N(c2ccc3c(c2)c2ccccc2n3-c2ccccc2)c2cccc3c2-c2ccccc2C32C3CC4CC(C3)CC2C4)cc1 ; c1ccc(-c2ccccc2-c2ccccc2-c2ccccc2N(c2ccc3sc4ccccc4c3c2)c2cccc3c2-c2ccccc2C32C3CC4CC(C3)CC2C4)cc1]

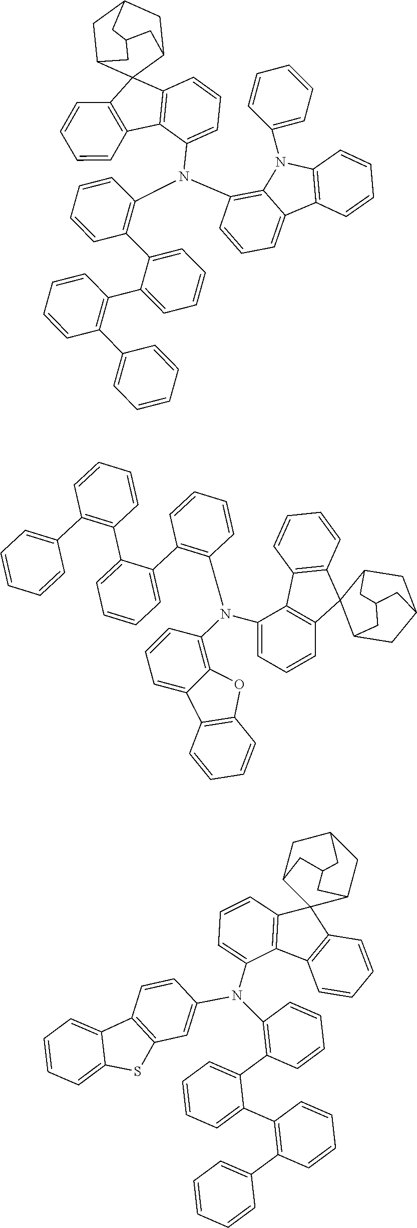 [SMILES: c1ccc(-c2ccccc2-c2ccccc2-c2ccccc2N(c2cccc3c2-c2ccccc2C32C3CC4CC(C3)CC2C4)c2cccc3c4ccccc4n(-c4ccccc4)c23)cc1 ; c1ccc(-c2ccccc2-c2ccccc2-c2ccccc2N(c2cccc3c2-c2ccccc2C32C3CC4CC(C3)CC2C4)c2cccc3c2oc2ccccc23)cc1 ; c1ccc(-c2ccccc2-c2ccccc2-c2ccccc2N(c2ccc3c(c2)sc2ccccc23)c2cccc3c2-c2ccccc2C32C3CC4CC(C3)CC2C4)cc1]

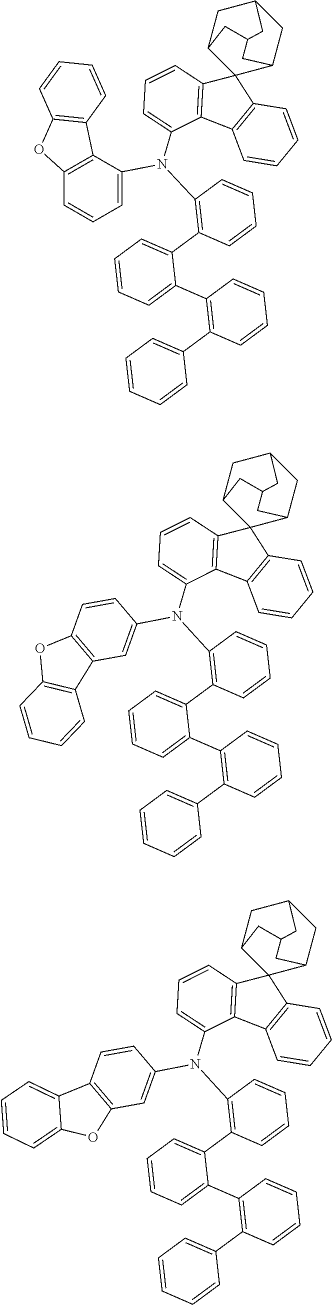 [SMILES: c1ccc(-c2ccccc2-c2ccccc2-c2ccccc2N(c2cccc3c2-c2ccccc2C32C3CC4CC(C3)CC2C4)c2cccc3oc4ccccc4c23)cc1 ; c1ccc(-c2ccccc2-c2ccccc2-c2ccccc2N(c2ccc3oc4ccccc4c3c2)c2cccc3c2-c2ccccc2C32C3CC4CC(C3)CC2C4)cc1 ; c1ccc(-c2ccccc2-c2ccccc2-c2ccccc2N(c2ccc3c(c2)oc2ccccc23)c2cccc3c2-c2ccccc2C32C3CC4CC(C3)CC2C4)cc1]

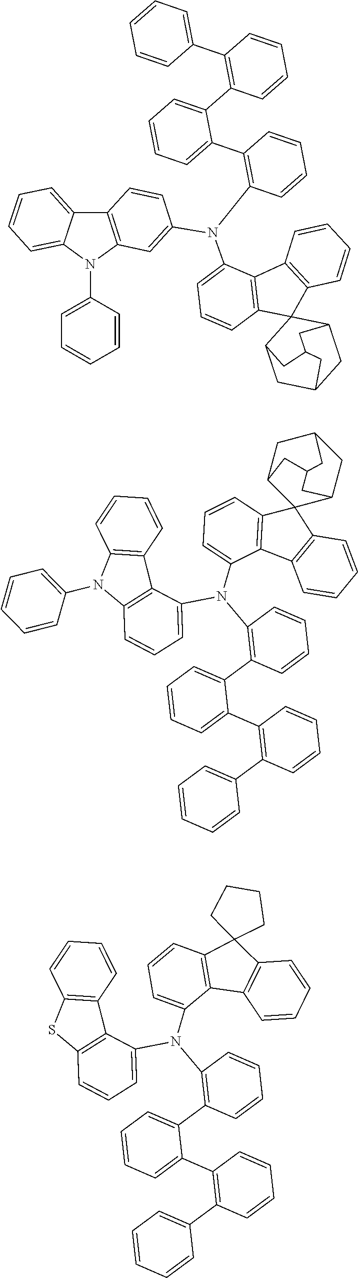

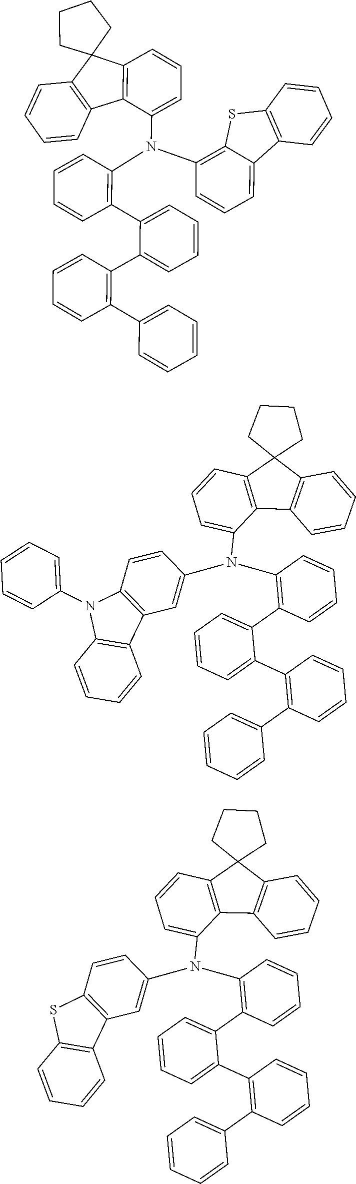

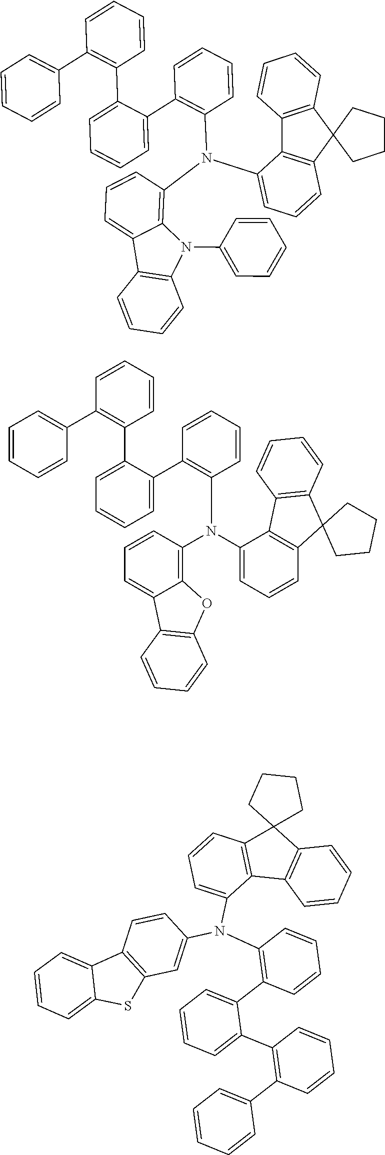

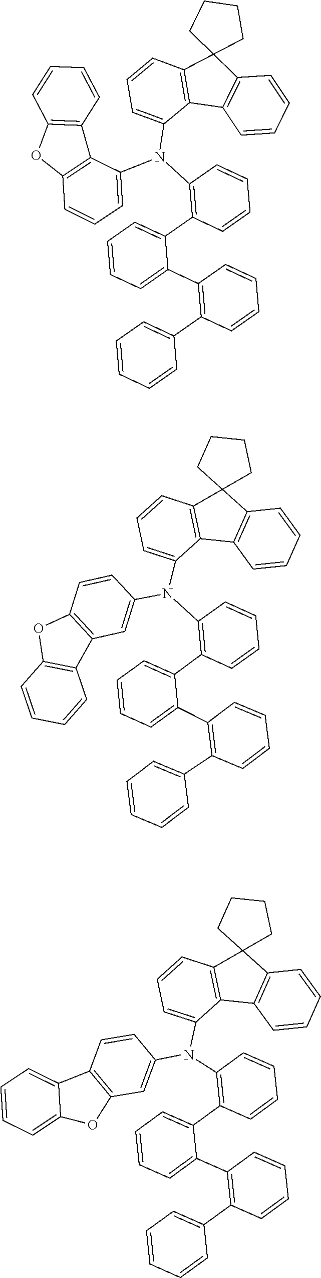 [SMILES: c1ccc(-c2ccccc2-c2ccccc2-c2ccccc2N(c2cccc3c2-c2ccccc2C32CCCC2)c2cccc3oc4ccccc4c23)cc1 ; c1ccc(-c2ccccc2-c2ccccc2-c2ccccc2N(c2ccc3oc4ccccc4c3c2)c2cccc3c2-c2ccccc2C32CCCC2)cc1 ; c1ccc(-c2ccccc2-c2ccccc2-c2ccccc2N(c2ccc3c(c2)oc2ccccc23)c2cccc3c2-c2ccccc2C32CCCC2)cc1]

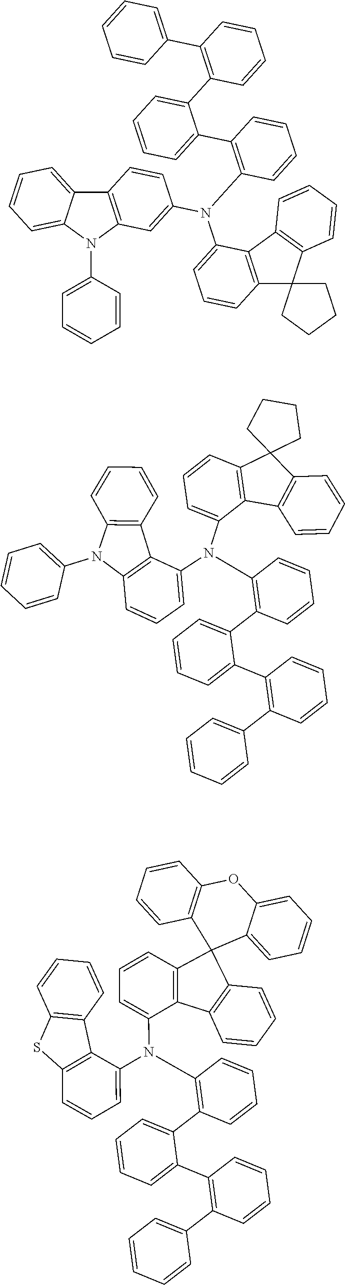 [SMILES: c1ccc(-c2ccccc2-c2ccccc2-c2ccccc2N(c2ccc3c4ccccc4n(-c4ccccc4)c3c2)c2cccc3c2-c2ccccc2C32CCCC2)cc1 ; c1ccc(-c2ccccc2-c2ccccc2-c2ccccc2N(c2cccc3c2-c2ccccc2C32CCCC2)c2cccc3c2c2ccccc2n3-c2ccccc2)cc1 ; c1ccc(-c2ccccc2-c2ccccc2-c2ccccc2N(c2cccc3c2-c2ccccc2C32c3ccccc3Oc3ccccc32)c2cccc3sc4ccccc4c23)cc1]

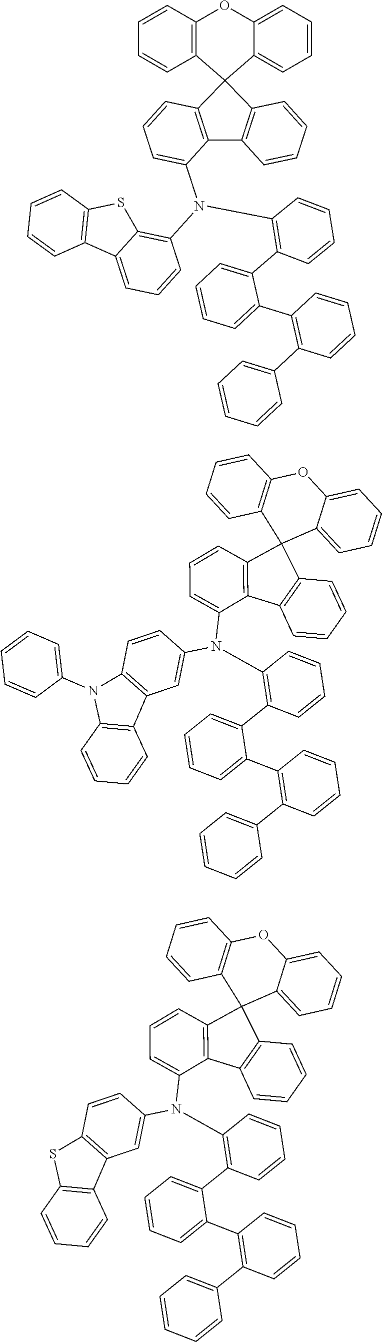

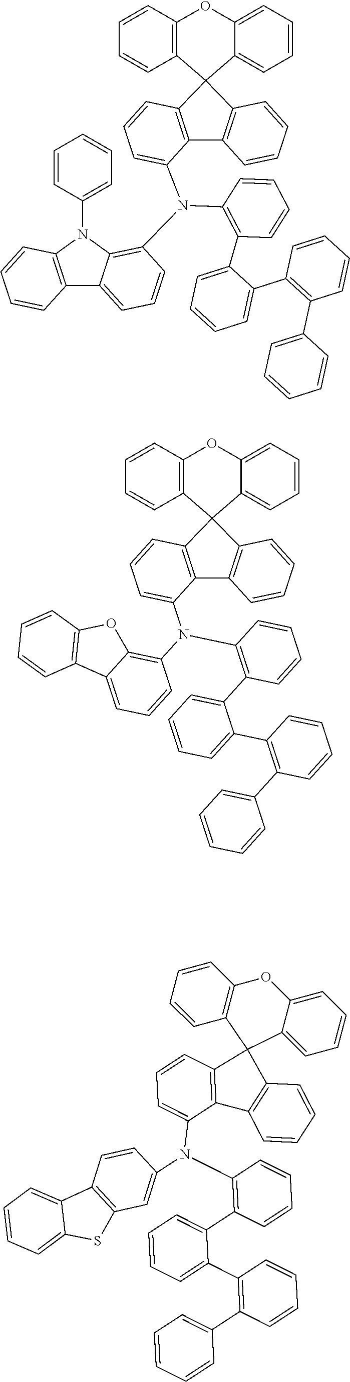 [SMILES: c1ccc(-c2ccccc2-c2ccccc2-c2ccccc2N(c2cccc3c2-c2ccccc2C32c3ccccc3Oc3ccccc32)c2cccc3c4ccccc4n(-c4ccccc4)c23)cc1 ; c1ccc(-c2ccccc2-c2ccccc2-c2ccccc2N(c2cccc3c2-c2ccccc2C32c3ccccc3Oc3ccccc32)c2cccc3c2oc2ccccc23)cc1 ; c1ccc(-c2ccccc2-c2ccccc2-c2ccccc2N(c2ccc3c(c2)sc2ccccc23)c2cccc3c2-c2ccccc2C32c3ccccc3Oc3ccccc32)cc1]

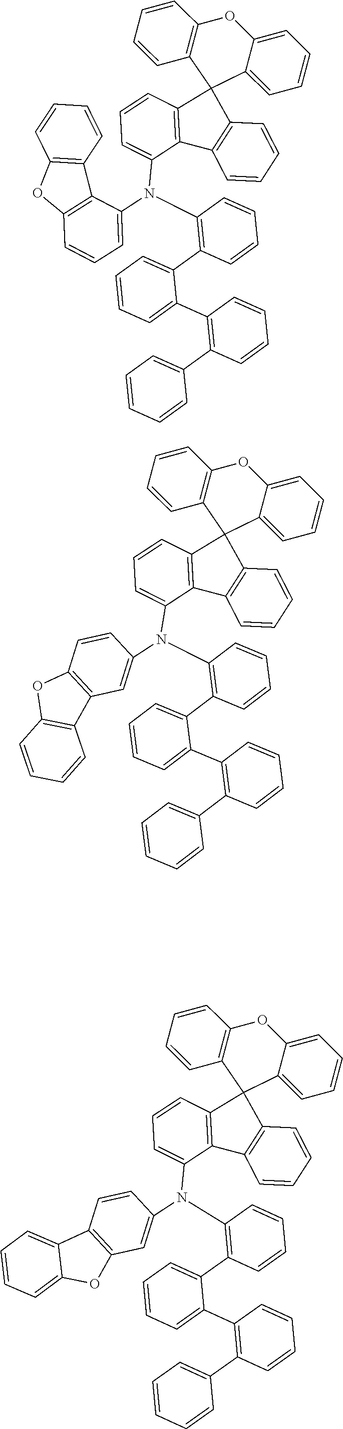

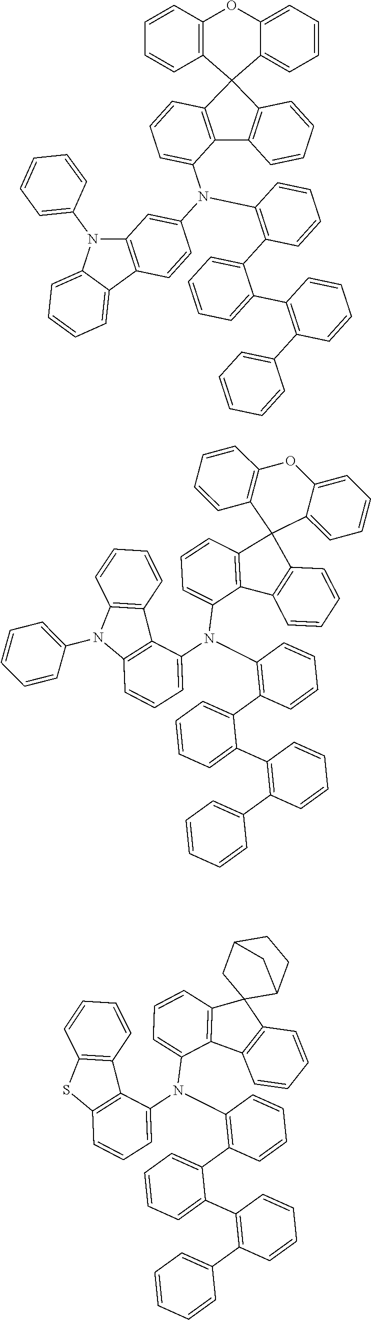

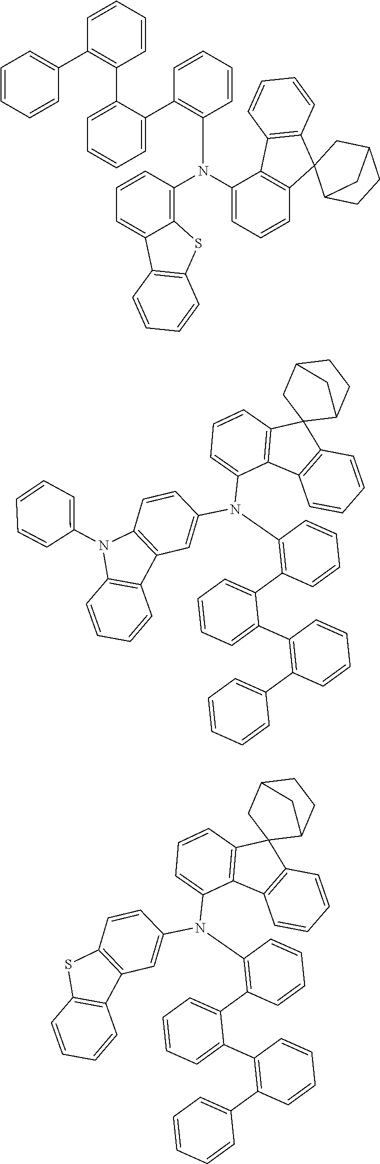

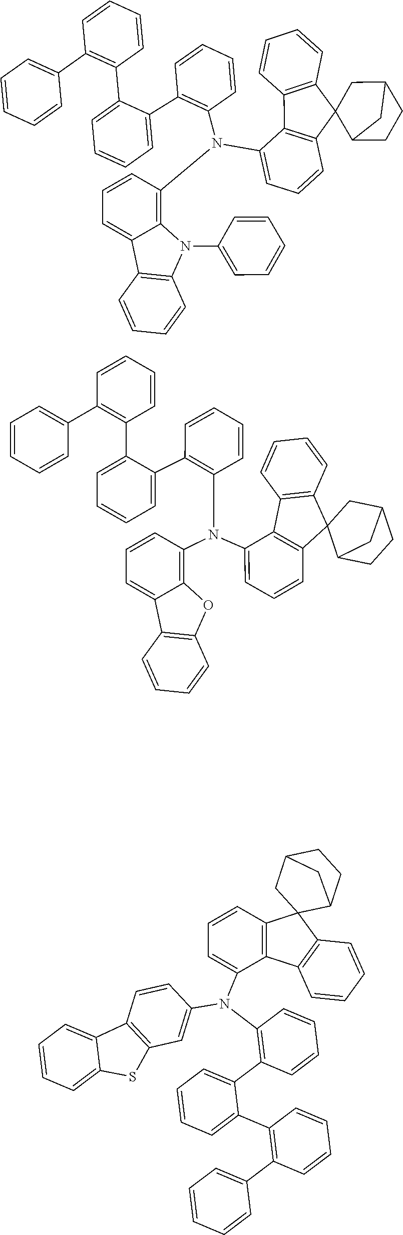

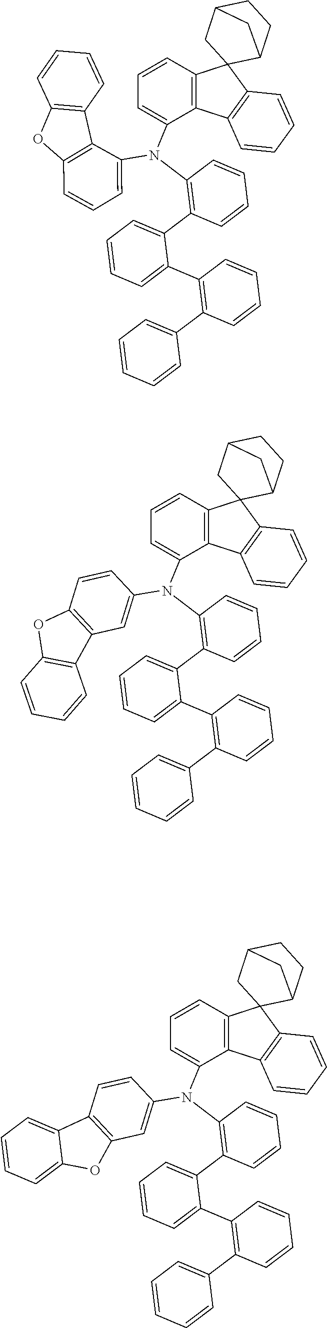 [SMILES: c1ccc(-c2ccccc2-c2ccccc2-c2ccccc2N(c2cccc3c2-c2ccccc2C32CC3CCC2C3)c2cccc3oc4ccccc4c23)cc1 ; c1ccc(-c2ccccc2-c2ccccc2-c2ccccc2N(c2ccc3oc4ccccc4c3c2)c2cccc3c2-c2ccccc2C32CC3CCC2C3)cc1 ; c1ccc(-c2ccccc2-c2ccccc2-c2ccccc2N(c2ccc3c(c2)oc2ccccc23)c2cccc3c2-c2ccccc2C32CC3CCC2C3)cc1]

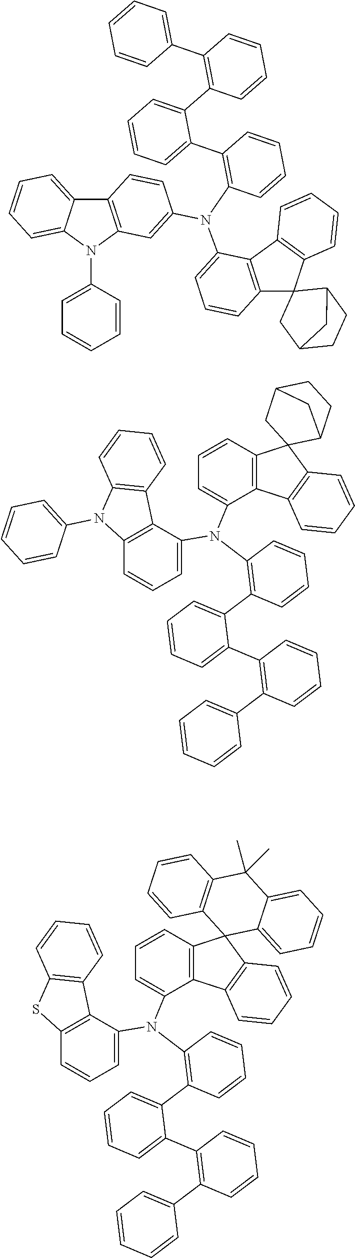

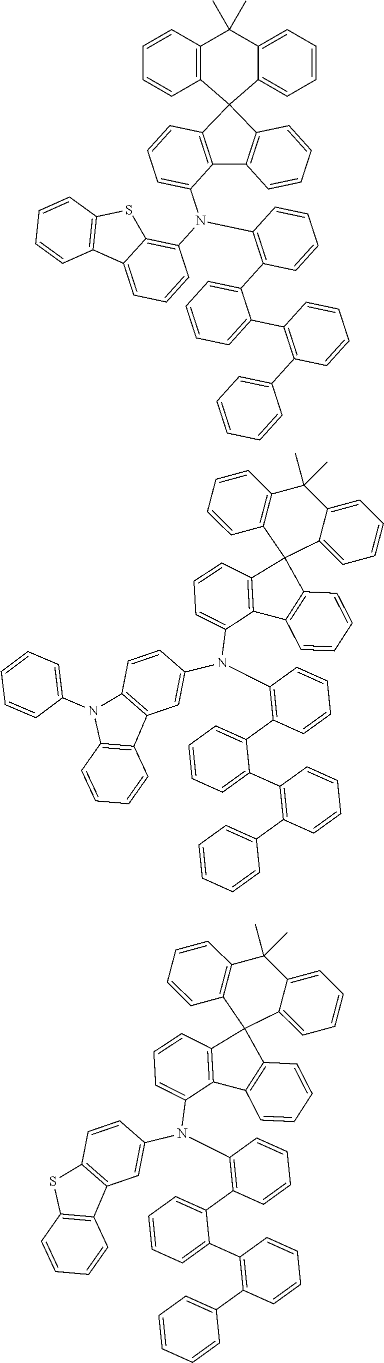

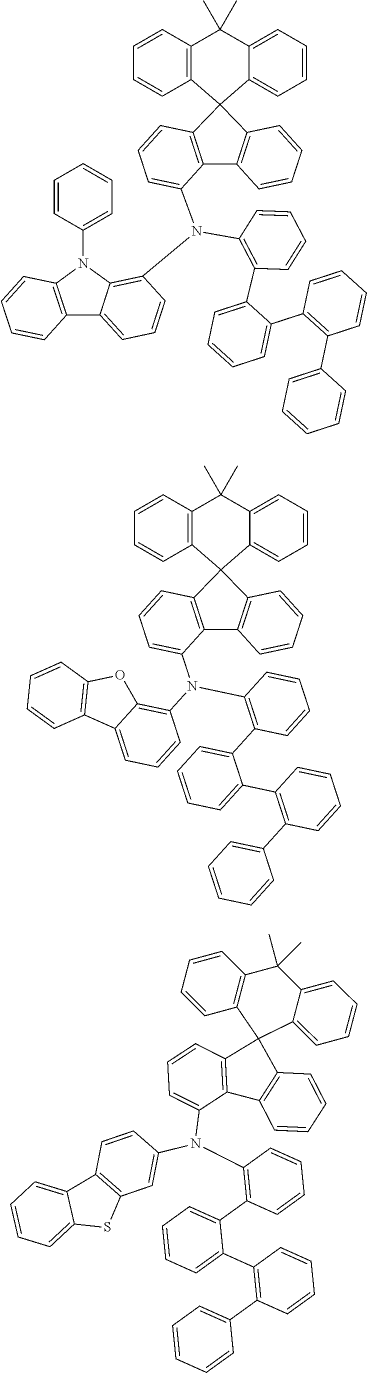

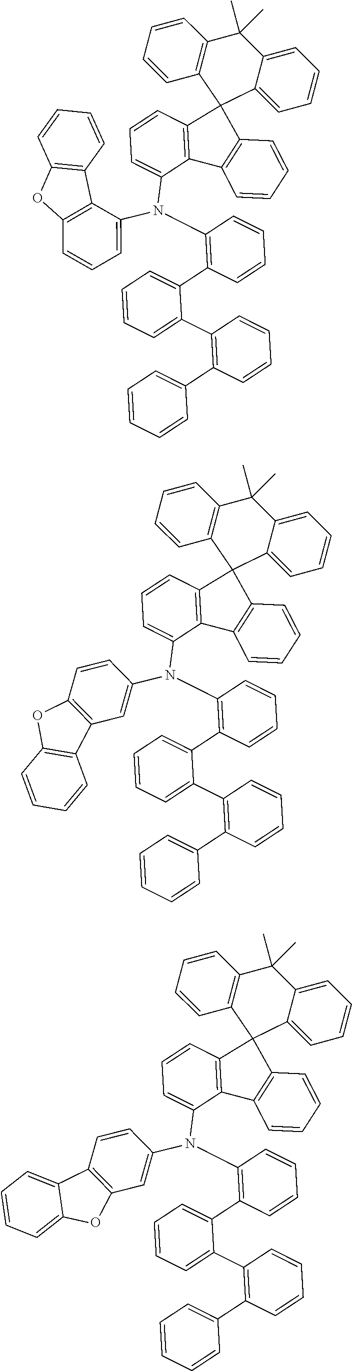 [SMILES: CC1(C)c2ccccc2C2(c3ccccc3-c3c(N(c4ccccc4-c4ccccc4-c4ccccc4-c4ccccc4)c4cccc5oc6ccccc6c45)cccc32)c2ccccc21 ; CC1(C)c2ccccc2C2(c3ccccc3-c3c(N(c4ccc5oc6ccccc6c5c4)c4ccccc4-c4ccccc4-c4ccccc4-c4ccccc4)cccc32)c2ccccc21 ; CC1(C)c2ccccc2C2(c3ccccc3-c3c(N(c4ccc5c(c4)oc4ccccc45)c4ccccc4-c4ccccc4-c4ccccc4-c4ccccc4)cccc32)c2ccccc21]

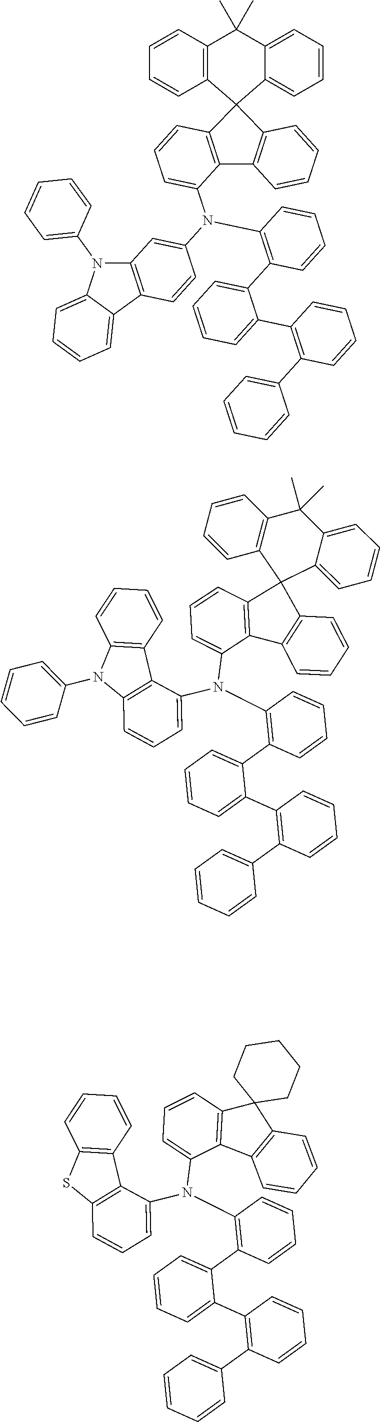

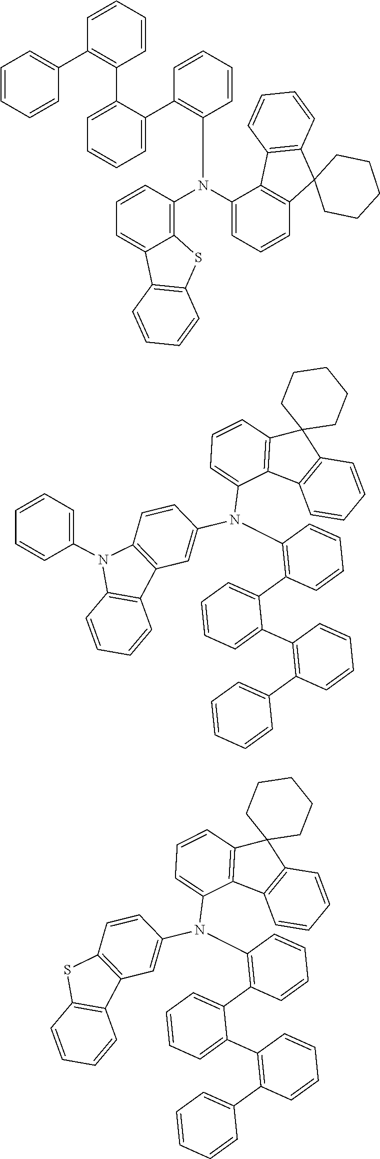

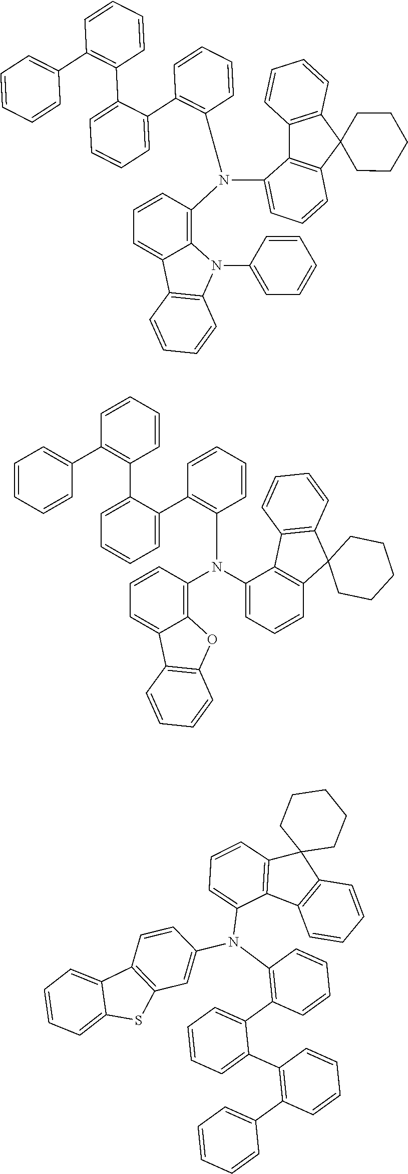 [SMILES: c1ccc(-c2ccccc2-c2ccccc2-c2ccccc2N(c2cccc3c2-c2ccccc2C32CCCCC2)c2cccc3c4ccccc4n(-c4ccccc4)c23)cc1 ; c1ccc(-c2ccccc2-c2ccccc2-c2ccccc2N(c2cccc3c2-c2ccccc2C32CCCCC2)c2cccc3c2oc2ccccc23)cc1 ; c1ccc(-c2ccccc2-c2ccccc2-c2ccccc2N(c2ccc3c(c2)sc2ccccc23)c2cccc3c2-c2ccccc2C32CCCCC2)cc1]

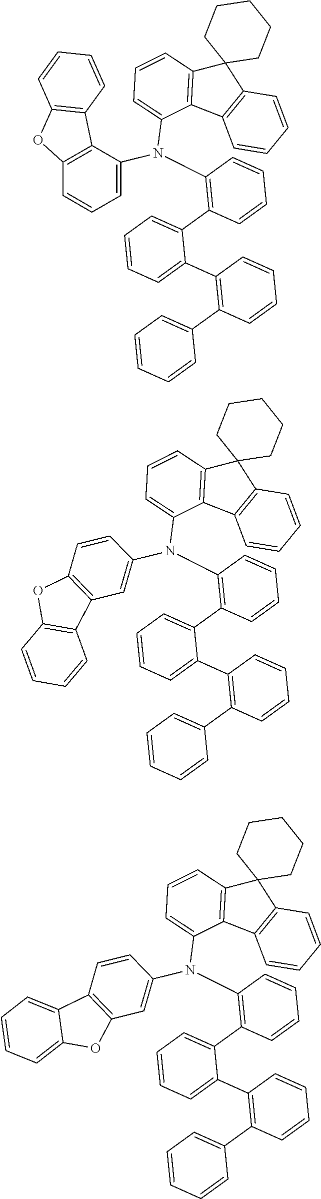 [SMILES: c1ccc(-c2ccccc2-c2ccccc2-c2ccccc2N(c2cccc3c2-c2ccccc2C32CCCCC2)c2cccc3oc4ccccc4c23)cc1 ; c1ccc(-c2ccccc2-c2ccccc2-c2ccccc2N(c2ccc3oc4ccccc4c3c2)c2cccc3c2-c2ccccc2C32CCCCC2)cc1 ; c1ccc(-c2ccccc2-c2ccccc2-c2ccccc2N(c2ccc3c(c2)oc2ccccc23)c2cccc3c2-c2ccccc2C32CCCCC2)cc1]

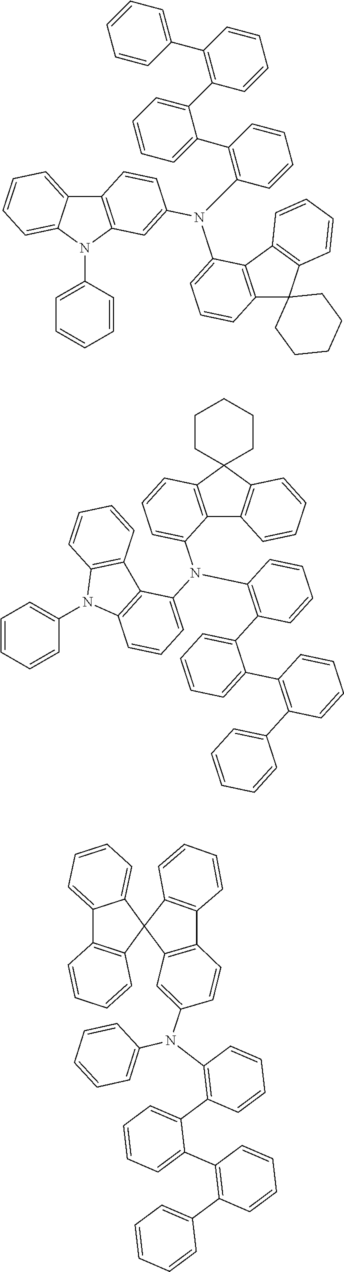 [SMILES: c1ccc(-c2ccccc2-c2ccccc2-c2ccccc2N(c2ccc3c4ccccc4n(-c4ccccc4)c3c2)c2cccc3c2-c2ccccc2C32CCCCC2)cc1 ; c1ccc(-c2ccccc2-c2ccccc2-c2ccccc2N(c2cccc3c2-c2ccccc2C32CCCCC2)c2cccc3c2c2ccccc2n3-c2ccccc2)cc1 ; c1ccc(-c2ccccc2-c2ccccc2-c2ccccc2N(c2ccccc2)c2ccc3c(c2)C2(c4ccccc4-c4ccccc42)c2ccccc2-3)cc1]

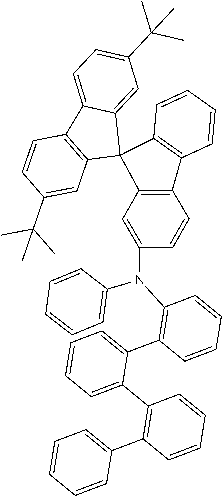 [SMILES: CC(C)(C)c1ccc2c(c1)C1(c3ccccc3-c3ccc(N(c4ccccc4)c4ccccc4-c4ccccc4-c4ccccc4-c4ccccc4)cc31)c1cc(C(C)(C)C)ccc1-2]

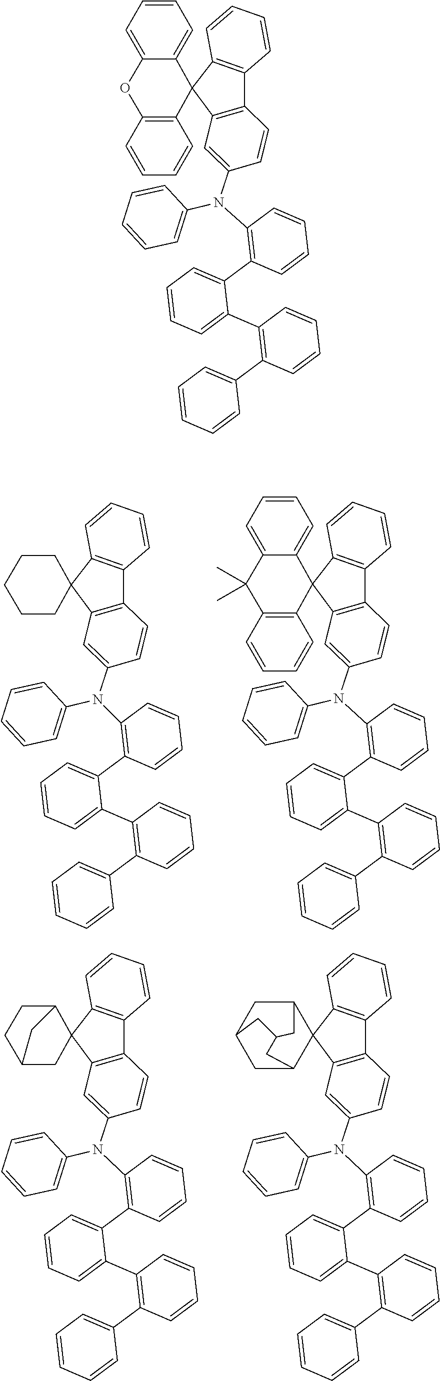

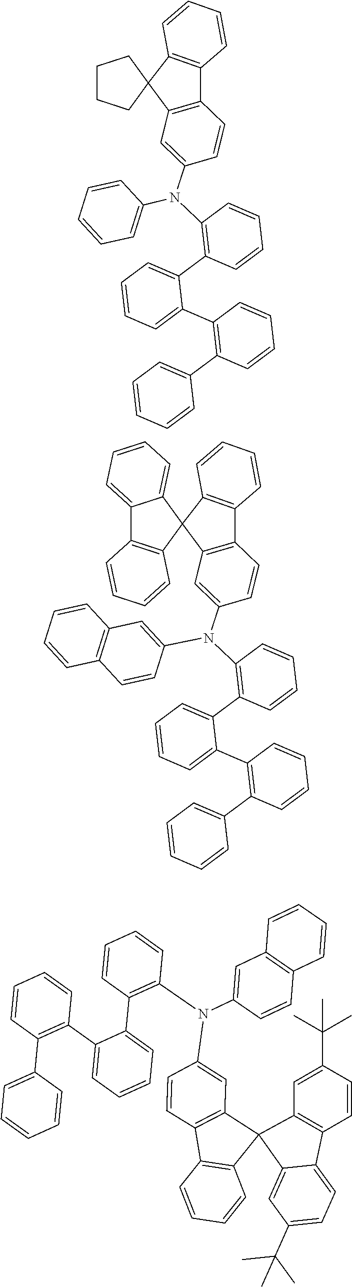

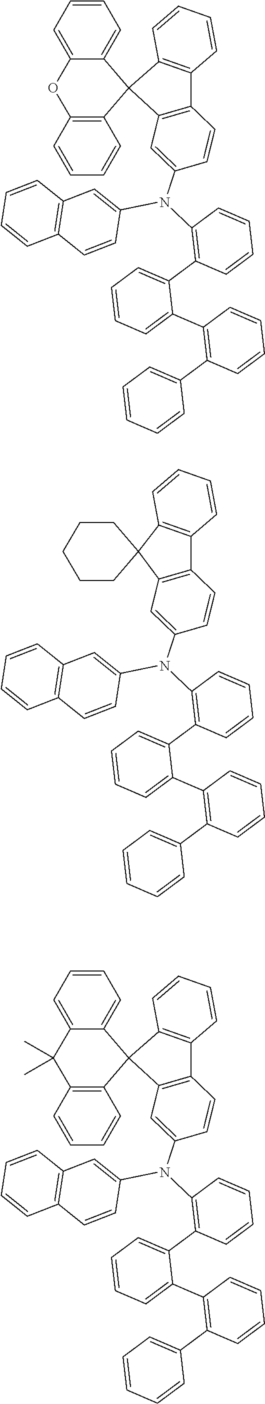

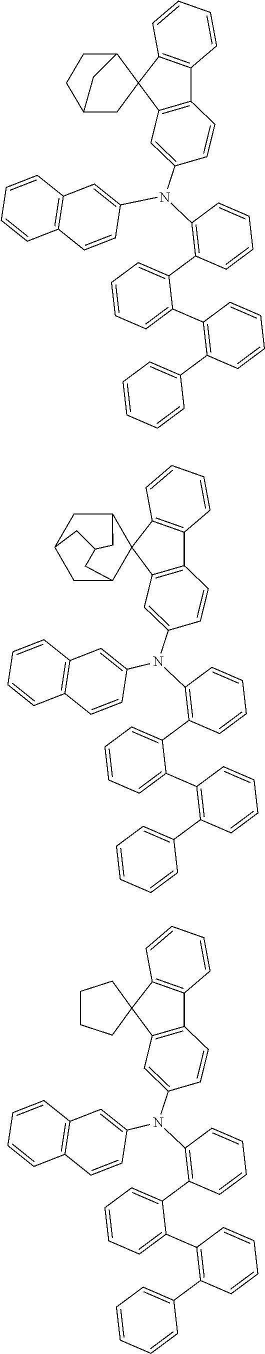

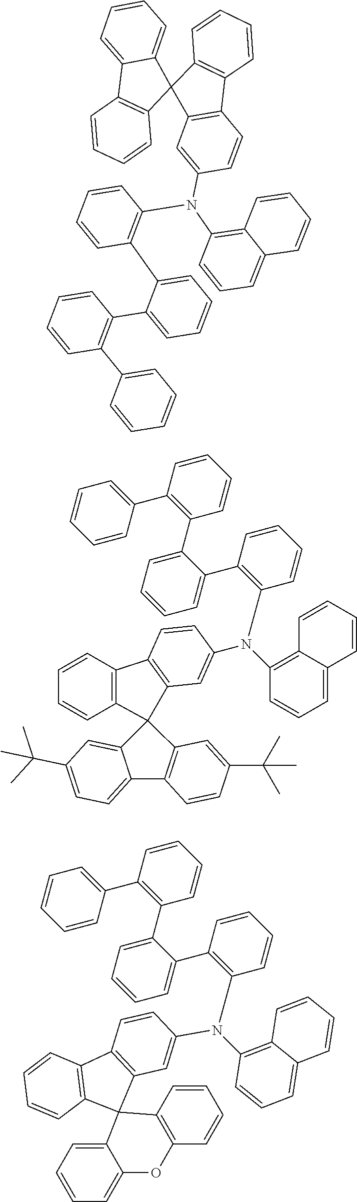

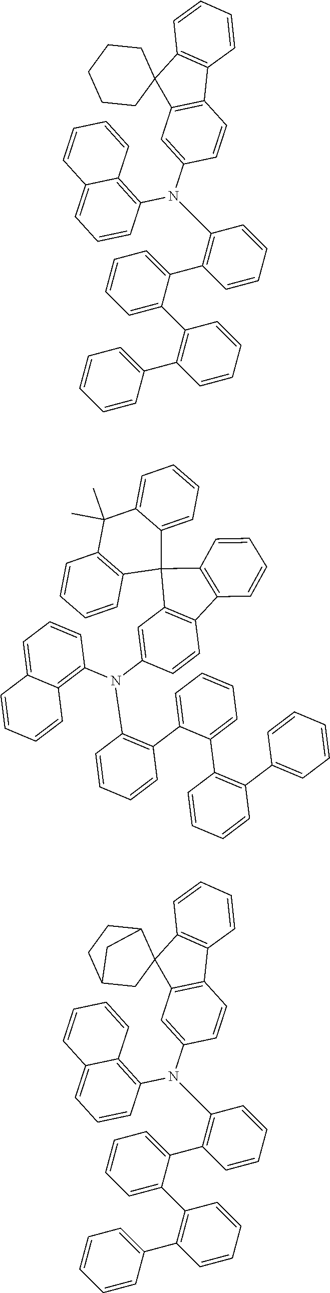 [SMILES: c1ccc(-c2ccccc2-c2ccccc2-c2ccccc2N(c2ccc3c(c2)C2(CCCCC2)c2ccccc2-3)c2cccc3ccccc23)cc1 ; CC1(C)c2ccccc2C2(c3ccccc3-c3ccc(N(c4ccccc4-c4ccccc4-c4ccccc4-c4ccccc4)c4cccc5ccccc45)cc32)c2ccccc21 ; c1ccc(-c2ccccc2-c2ccccc2-c2ccccc2N(c2ccc3c(c2)C2(CC4CCC2C4)c2ccccc2-3)c2cccc3ccccc23)cc1]

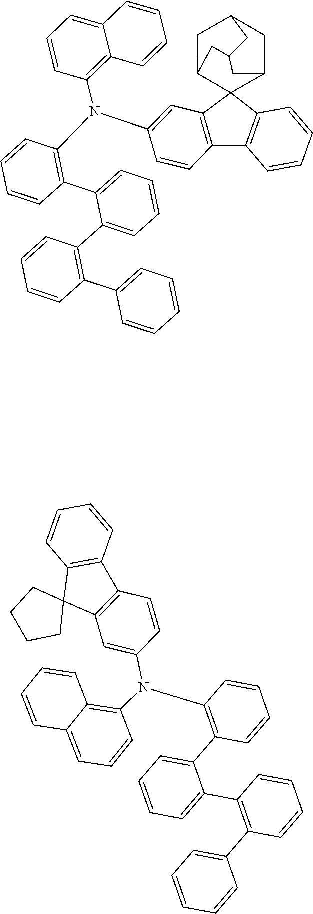 [SMILES: c1ccc(-c2ccccc2-c2ccccc2-c2ccccc2N(c2ccc3c(c2)C2(c4ccccc4-3)C3CC4CC(C3)CC2C4)c2cccc3ccccc23)cc1 ; c1ccc(-c2ccccc2-c2ccccc2-c2ccccc2N(c2ccc3c(c2)C2(CCCC2)c2ccccc2-3)c2cccc3ccccc23)cc1]

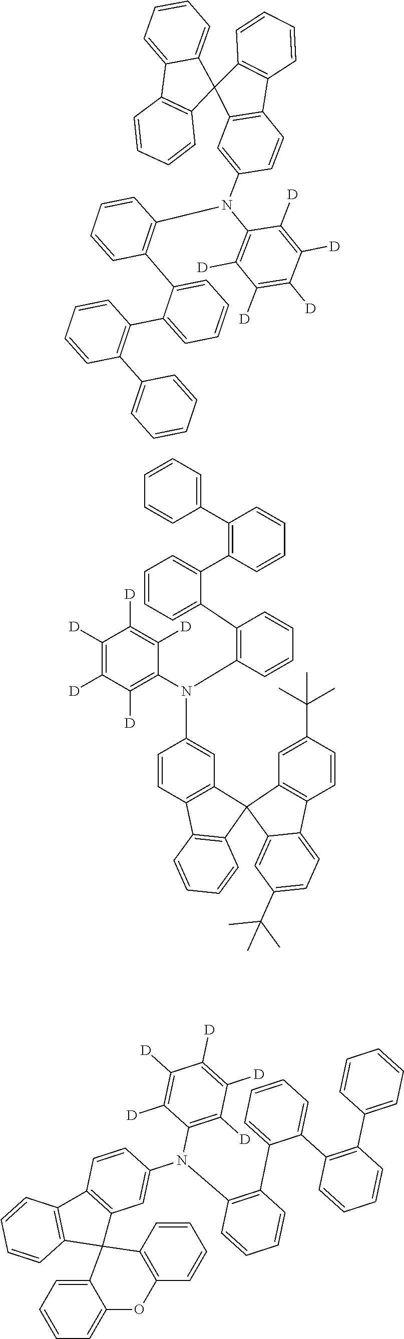

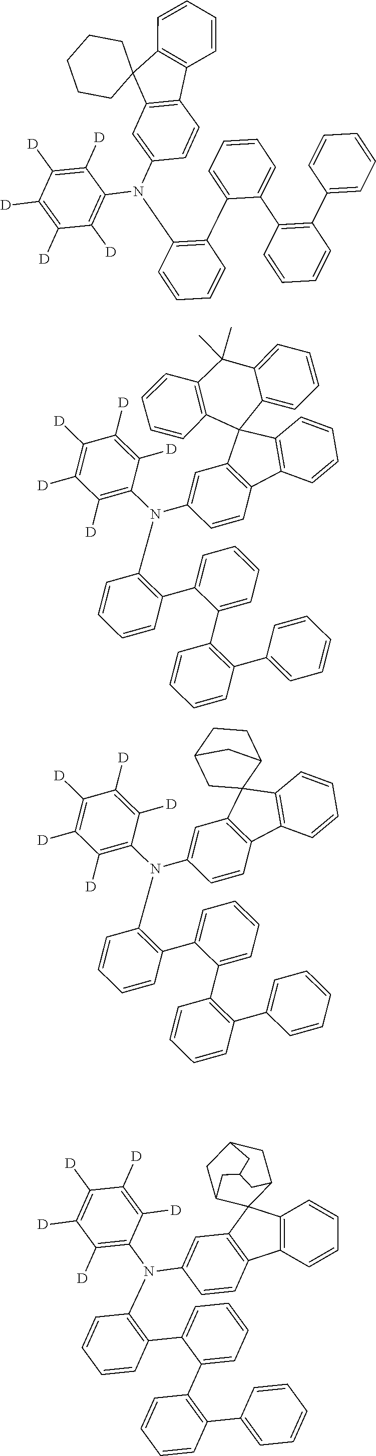

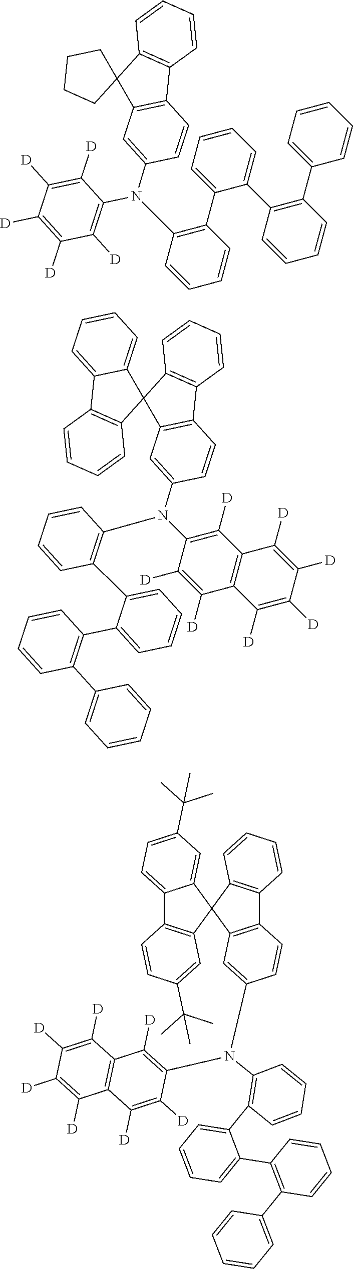

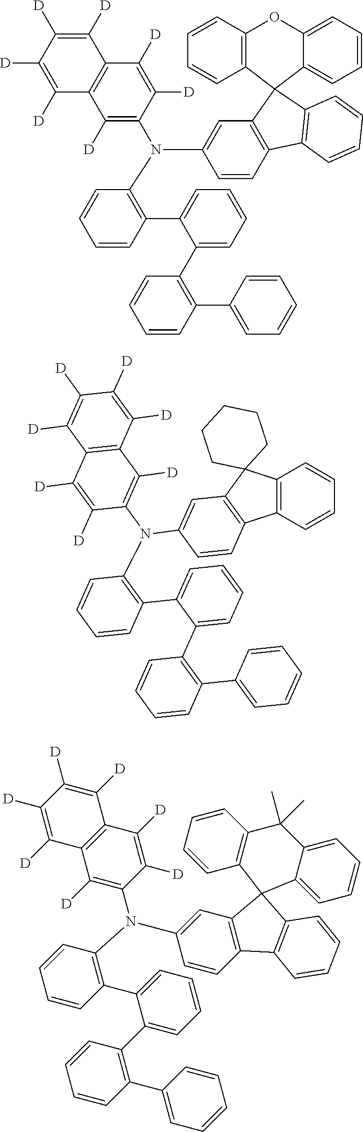

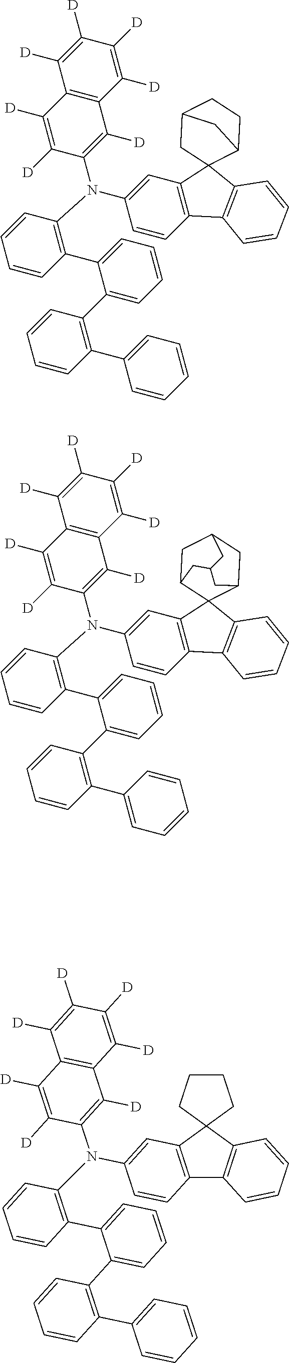 [SMILES: [2H]c1c([2H])c([2H])c2c([2H])c(N(c3ccc4c(c3)C3(CC5CCC3C5)c3ccccc3-4)c3ccccc3-c3ccccc3-c3ccccc3-c3ccccc3)c([2H])c([2H])c2c1[2H] ; [2H]c1c([2H])c([2H])c2c([2H])c(N(c3ccc4c(c3)C3(c5ccccc5-4)C4CC5CC(C4)CC3C5)c3ccccc3-c3ccccc3-c3ccccc3-c3ccccc3)c([2H])c([2H])c2c1[2H] ; [2H]c1c([2H])c([2H])c2c([2H])c(N(c3ccc4c(c3)C3(CCCC3)c3ccccc3-4)c3ccccc3-c3ccccc3-c3ccccc3-c3ccccc3)c([2H])c([2H])c2c1[2H]]

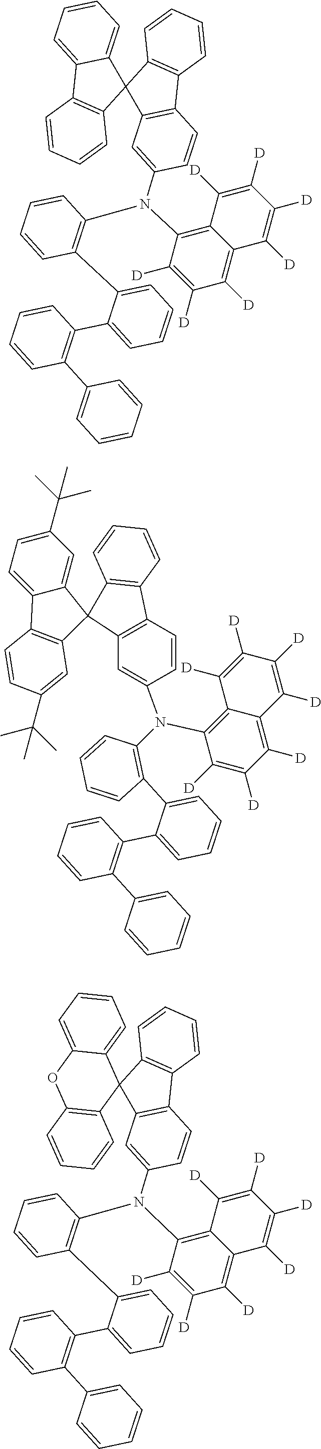

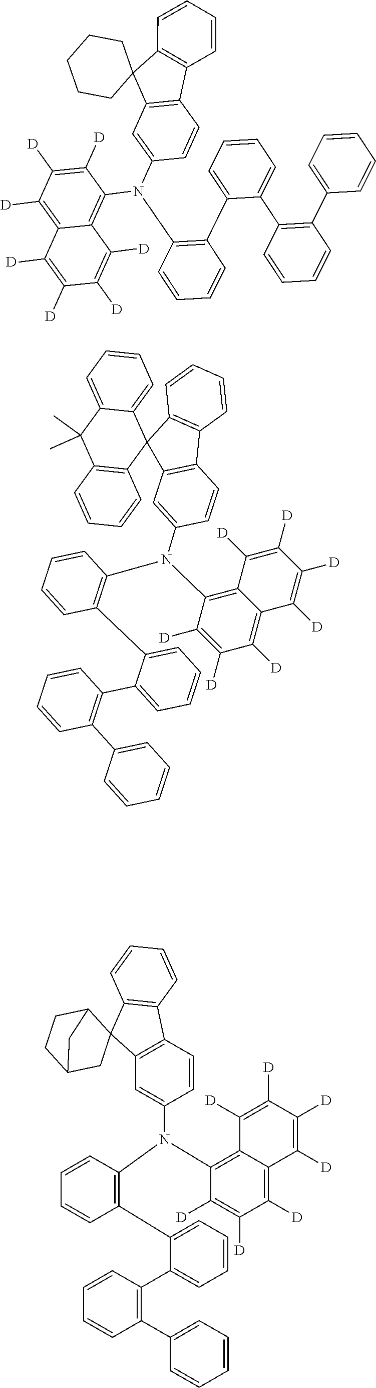

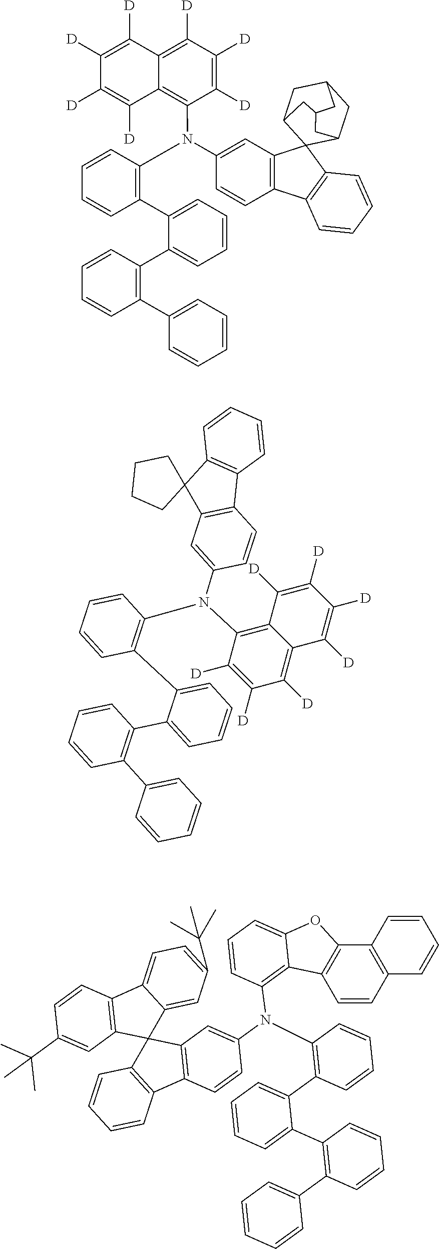 [SMILES: [2H]c1c([2H])c([2H])c2c(N(c3ccc4c(c3)C3(c5ccccc5-4)C4CC5CC(C4)CC3C5)c3ccccc3-c3ccccc3-c3ccccc3-c3ccccc3)c([2H])c([2H])c([2H])c2c1[2H] ; [2H]c1c([2H])c([2H])c2c(N(c3ccc4c(c3)C3(CCCC3)c3ccccc3-4)c3ccccc3-c3ccccc3-c3ccccc3-c3ccccc3)c([2H])c([2H])c([2H])c2c1[2H] ; CC(C)(C)c1ccc2c(c1)C1(c3ccccc3-c3ccc(N(c4ccccc4-c4ccccc4-c4ccccc4-c4ccccc4)c4cccc5oc6c7ccccc7ccc6c45)cc31)c1cc(C(C)(C)C)ccc1-2]

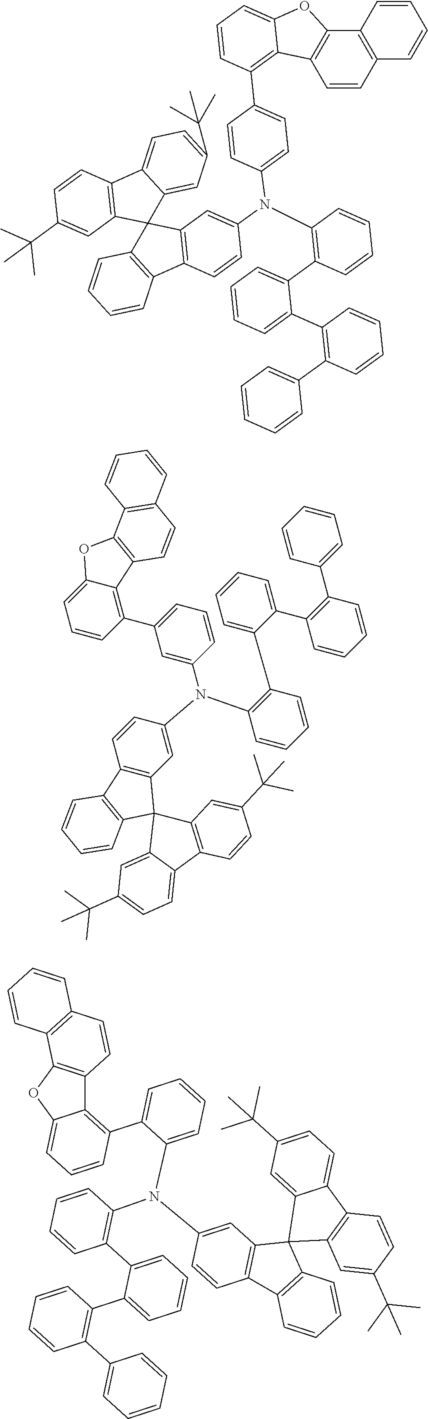

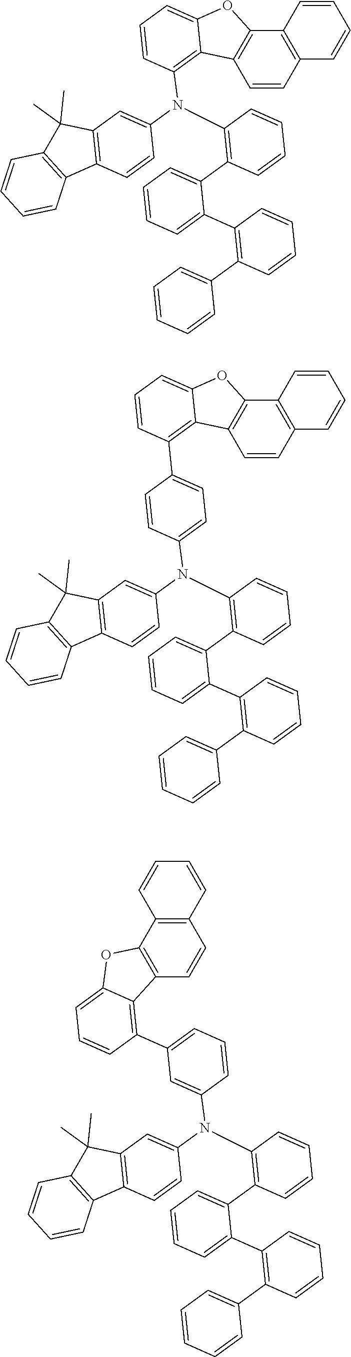 [SMILES: CC1(C)c2ccccc2-c2ccc(N(c3ccccc3-c3ccccc3-c3ccccc3-c3ccccc3)c3cccc4oc5c6ccccc6ccc5c34)cc21 ; CC1(C)c2ccccc2-c2ccc(N(c3ccc(-c4cccc5oc6c7ccccc7ccc6c45)cc3)c3ccccc3-c3ccccc3-c3ccccc3-c3ccccc3)cc21 ; CC1(C)c2ccccc2-c2ccc(N(c3cccc(-c4cccc5oc6c7ccccc7ccc6c45)c3)c3ccccc3-c3ccccc3-c3ccccc3-c3ccccc3)cc21]

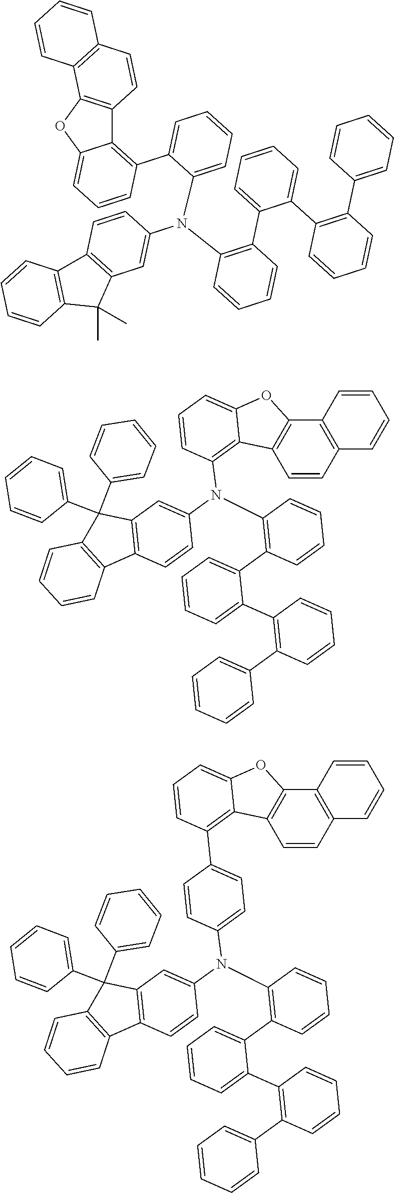 [SMILES: CC1(C)c2ccccc2-c2ccc(N(c3ccccc3-c3ccccc3-c3ccccc3-c3ccccc3)c3ccccc3-c3cccc4oc5c6ccccc6ccc5c34)cc21 ; c1ccc(-c2ccccc2-c2ccccc2-c2ccccc2N(c2ccc3c(c2)C(c2ccccc2)(c2ccccc2)c2ccccc2-3)c2cccc3oc4c5ccccc5ccc4c23)cc1 ; c1ccc(-c2ccccc2-c2ccccc2-c2ccccc2N(c2ccc(-c3cccc4oc5c6ccccc6ccc5c34)cc2)c2ccc3c(c2)C(c2ccccc2)(c2ccccc2)c2ccccc2-3)cc1]

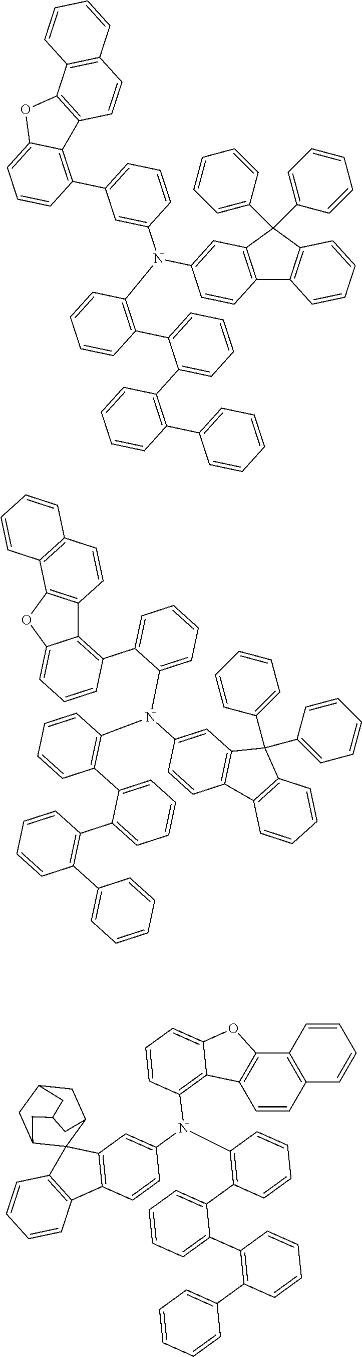 [SMILES: c1ccc(-c2ccccc2-c2ccccc2-c2ccccc2N(c2cccc(-c3cccc4oc5c6ccccc6ccc5c34)c2)c2ccc3c(c2)C(c2ccccc2)(c2ccccc2)c2ccccc2-3)cc1 ; c1ccc(-c2ccccc2-c2ccccc2-c2ccccc2N(c2ccc3c(c2)C(c2ccccc2)(c2ccccc2)c2ccccc2-3)c2ccccc2-c2cccc3oc4c5ccccc5ccc4c23)cc1 ; c1ccc(-c2ccccc2-c2ccccc2-c2ccccc2N(c2ccc3c(c2)C2(c4ccccc4-3)C3CC4CC(C3)CC2C4)c2cccc3oc4c5ccccc5ccc4c23)cc1]

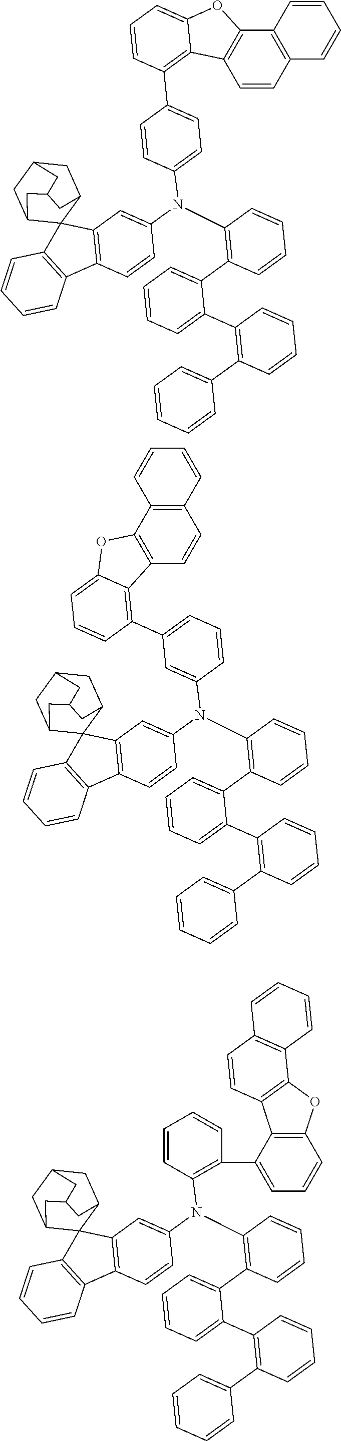

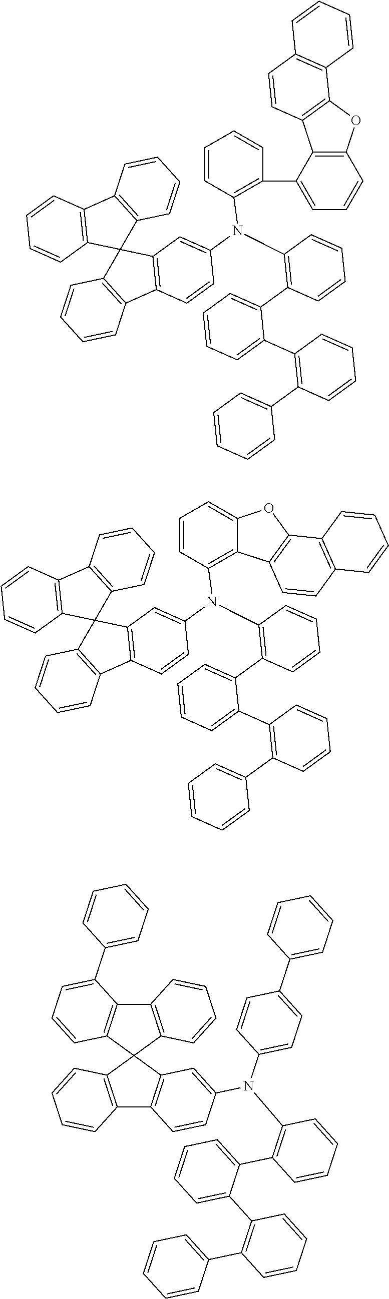 [SMILES: c1ccc(-c2ccccc2-c2ccccc2-c2ccccc2N(c2ccc3c(c2)C2(c4ccccc4-c4ccccc42)c2ccccc2-3)c2ccccc2-c2cccc3oc4c5ccccc5ccc4c23)cc1 ; c1ccc(-c2ccccc2-c2ccccc2-c2ccccc2N(c2ccc3c(c2)C2(c4ccccc4-c4ccccc42)c2ccccc2-3)c2cccc3oc4c5ccccc5ccc4c23)cc1 ; c1ccc(-c2ccc(N(c3ccc4c(c3)C3(c5ccccc5-4)c4ccccc4-c4c(-c5ccccc5)cccc43)c3ccccc3-c3ccccc3-c3ccccc3-c3ccccc3)cc2)cc1]

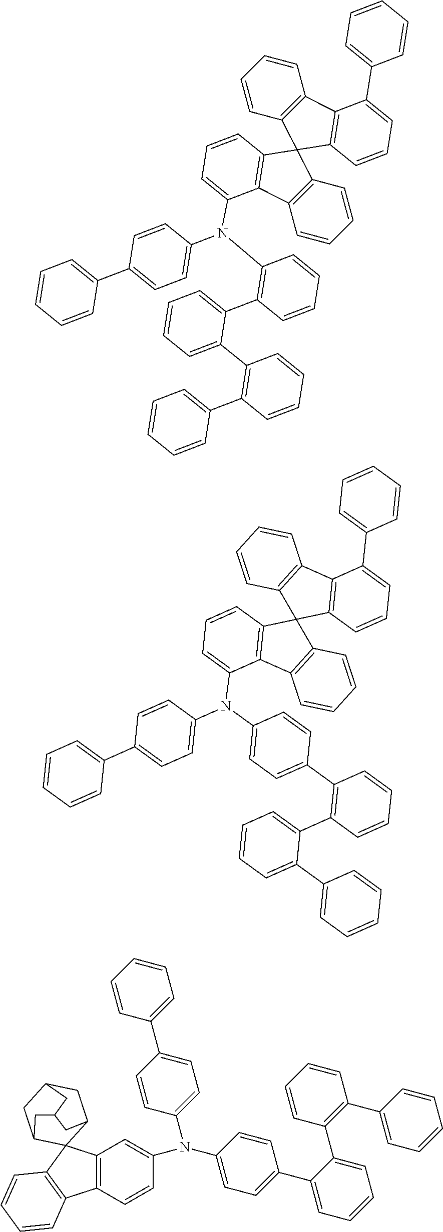 [SMILES: c1ccc(-c2ccc(N(c3ccccc3-c3ccccc3-c3ccccc3-c3ccccc3)c3cccc4c3-c3ccccc3C43c4ccccc4-c4c(-c5ccccc5)cccc43)cc2)cc1 ; c1ccc(-c2ccc(N(c3ccc(-c4ccccc4-c4ccccc4-c4ccccc4)cc3)c3cccc4c3-c3ccccc3C43c4ccccc4-c4c(-c5ccccc5)cccc43)cc2)cc1 ; c1ccc(-c2ccc(N(c3ccc(-c4ccccc4-c4ccccc4-c4ccccc4)cc3)c3ccc4c(c3)C3(c5ccccc5-4)C4CC5CC(C4)CC3C5)cc2)cc1]

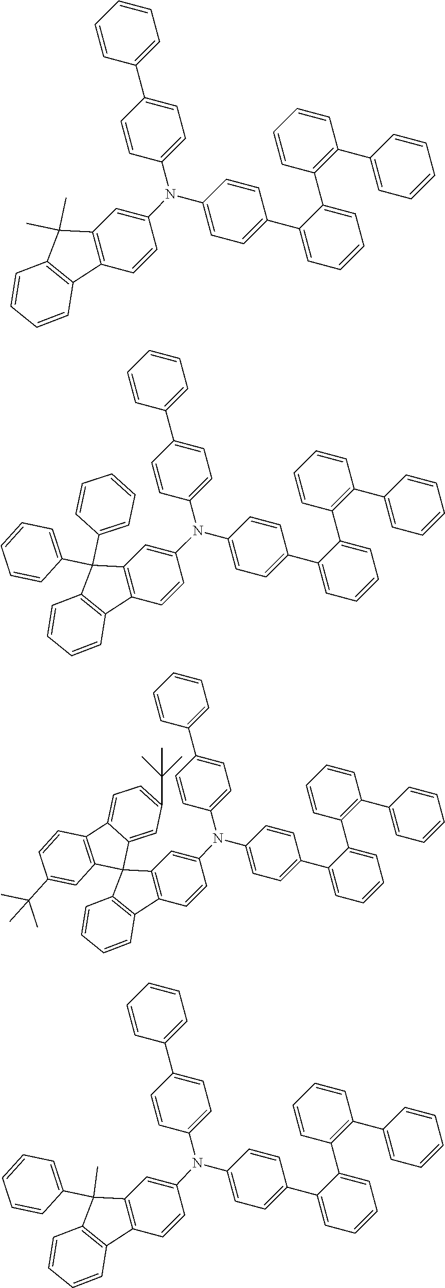 [SMILES: CC1(C)c2ccccc2-c2ccc(N(c3ccc(-c4ccccc4)cc3)c3ccc(-c4ccccc4-c4ccccc4-c4ccccc4)cc3)cc21 ; c1ccc(-c2ccc(N(c3ccc(-c4ccccc4-c4ccccc4-c4ccccc4)cc3)c3ccc4c(c3)C(c3ccccc3)(c3ccccc3)c3ccccc3-4)cc2)cc1 ; CC(C)(C)c1ccc2c(c1)C1(c3ccccc3-c3ccc(N(c4ccc(-c5ccccc5)cc4)c4ccc(-c5ccccc5-c5ccccc5-c5ccccc5)cc4)cc31)c1cc(C(C)(C)C)ccc1-2 ; CC1(c2ccccc2)c2ccccc2-c2ccc(N(c3ccc(-c4ccccc4)cc3)c3ccc(-c4ccccc4-c4ccccc4-c4ccccc4)cc3)cc21]

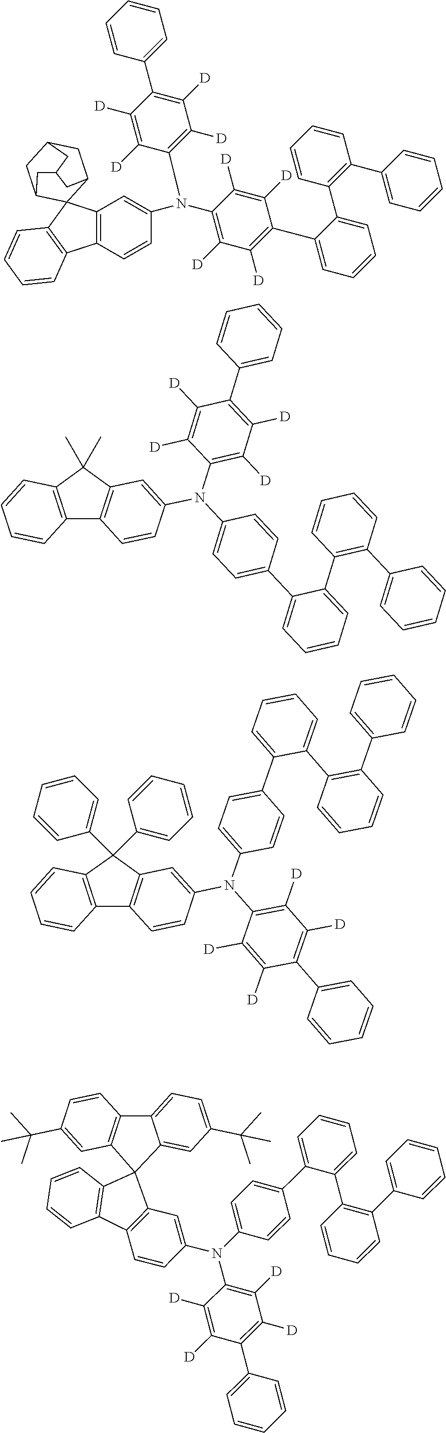

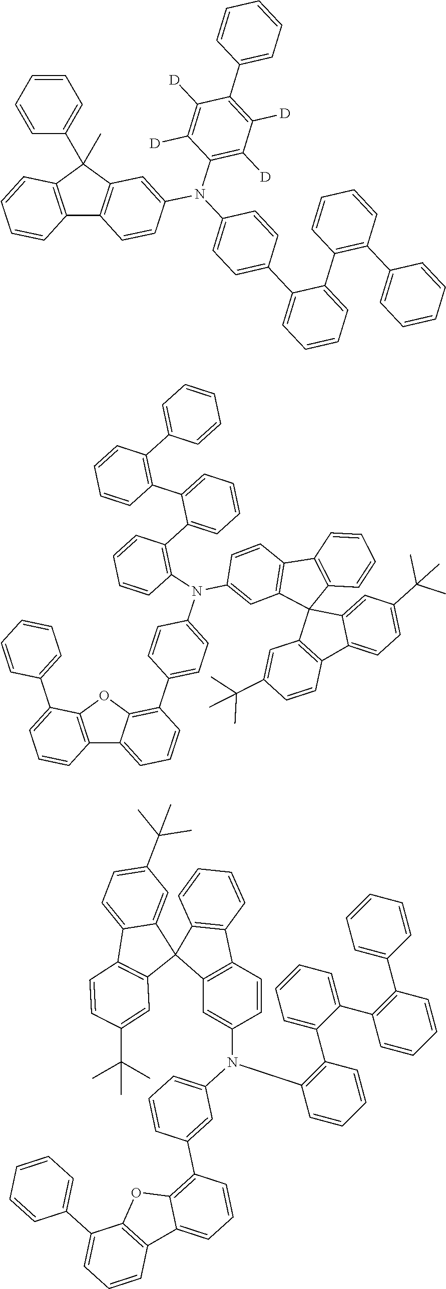

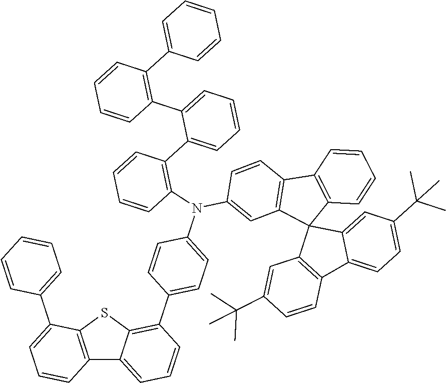 [SMILES: CC(C)(C)c1ccc2c(c1)C1(c3ccccc3-c3ccc(N(c4ccc(-c5cccc6c5sc5c(-c7ccccc7)cccc56)cc4)c4ccccc4-c4ccccc4-c4ccccc4-c4ccccc4)cc31)c1cc(C(C)(C)C)ccc1-2]

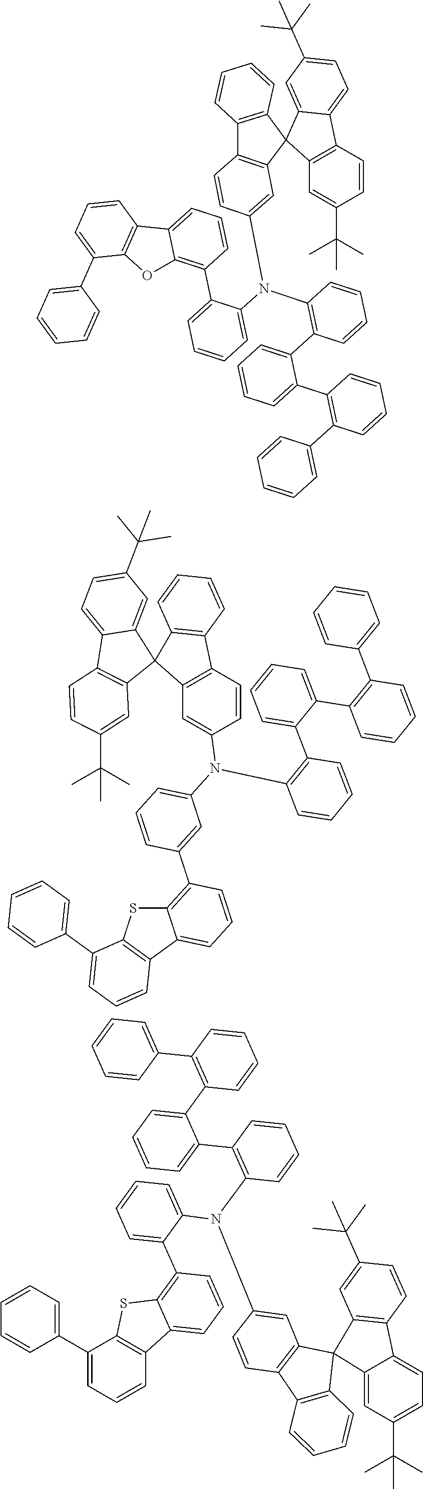

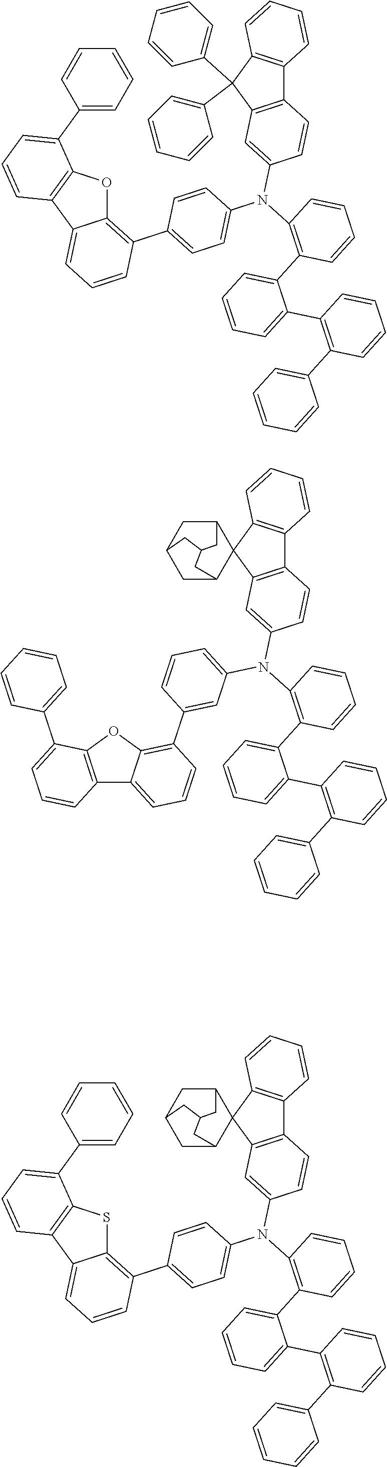

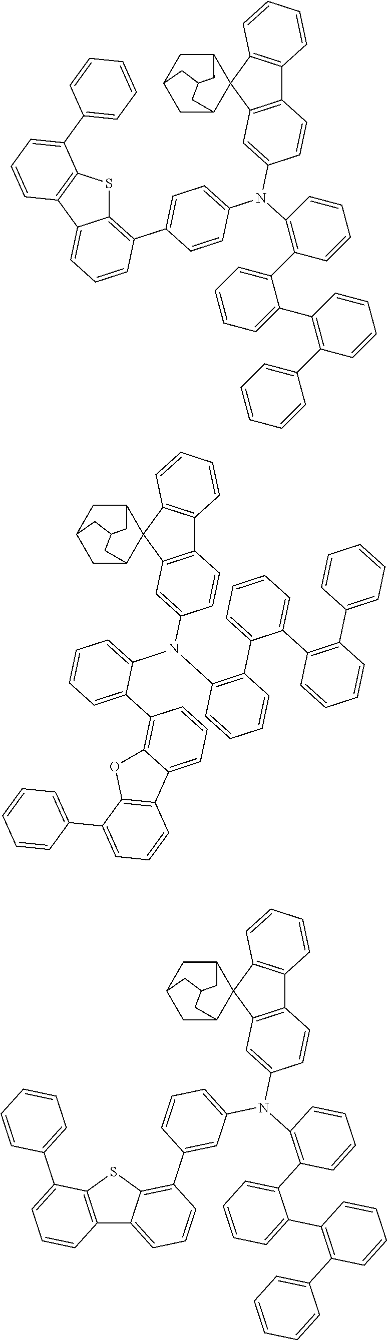 [SMILES: c1ccc(-c2ccccc2-c2ccccc2-c2ccccc2N(c2ccc(-c3cccc4c3sc3c(-c5ccccc5)cccc34)cc2)c2ccc3c(c2)C2(c4ccccc4-3)C3CC4CC(C3)CC2C4)cc1 ; c1ccc(-c2ccccc2-c2ccccc2-c2ccccc2N(c2ccc3c(c2)C2(c4ccccc4-3)C3CC4CC(C3)CC2C4)c2ccccc2-c2cccc3c2oc2c(-c4ccccc4)cccc23)cc1 ; c1ccc(-c2ccccc2-c2ccccc2-c2ccccc2N(c2cccc(-c3cccc4c3sc3c(-c5ccccc5)cccc34)c2)c2ccc3c(c2)C2(c4ccccc4-3)C3CC4CC(C3)CC2C4)cc1]

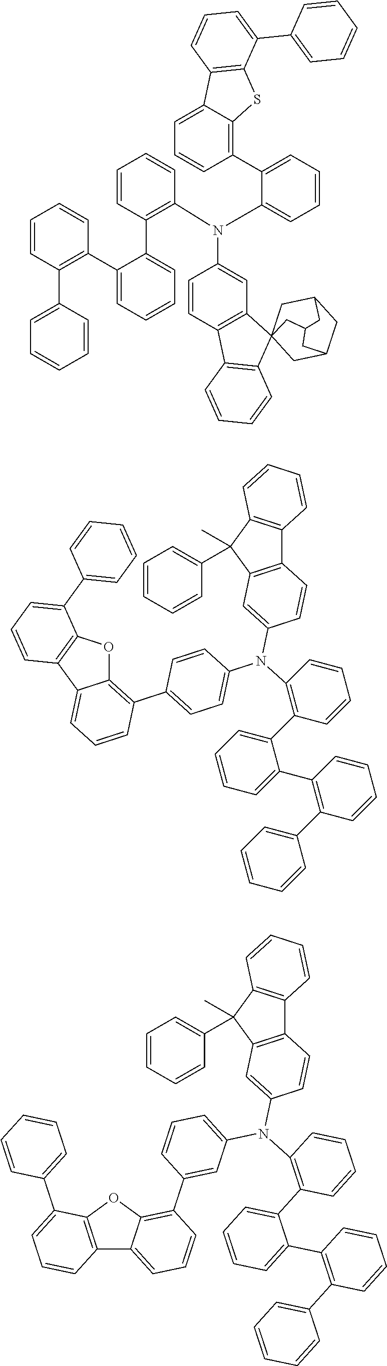

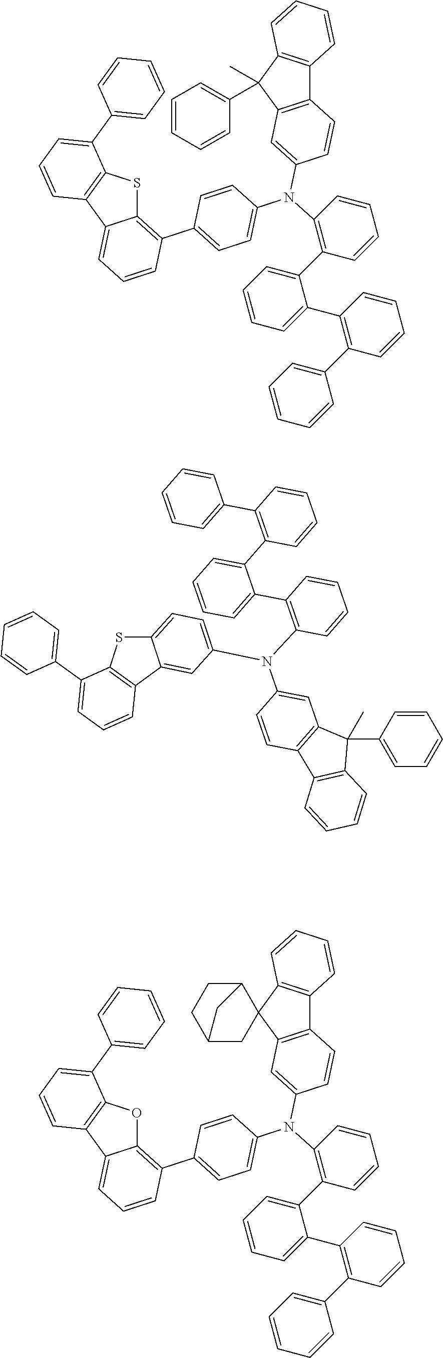 [SMILES: CC1(c2ccccc2)c2ccccc2-c2ccc(N(c3ccc(-c4cccc5c4sc4c(-c6ccccc6)cccc45)cc3)c3ccccc3-c3ccccc3-c3ccccc3-c3ccccc3)cc21 ; CC1(c2ccccc2)c2ccccc2-c2ccc(N(c3ccc4sc5c(-c6ccccc6)cccc5c4c3)c3ccccc3-c3ccccc3-c3ccccc3-c3ccccc3)cc21 ; c1ccc(-c2ccccc2-c2ccccc2-c2ccccc2N(c2ccc(-c3cccc4c3oc3c(-c5ccccc5)cccc34)cc2)c2ccc3c(c2)C2(CC4CCC2C4)c2ccccc2-3)cc1]

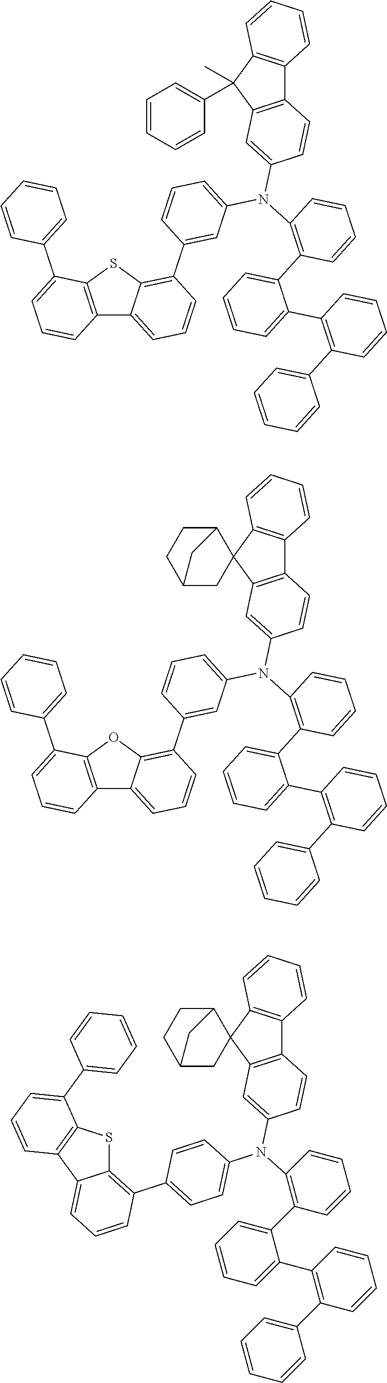 [SMILES: CC1(c2ccccc2)c2ccccc2-c2ccc(N(c3cccc(-c4cccc5c4sc4c(-c6ccccc6)cccc45)c3)c3ccccc3-c3ccccc3-c3ccccc3-c3ccccc3)cc21 ; c1ccc(-c2ccccc2-c2ccccc2-c2ccccc2N(c2cccc(-c3cccc4c3oc3c(-c5ccccc5)cccc34)c2)c2ccc3c(c2)C2(CC4CCC2C4)c2ccccc2-3)cc1 ; c1ccc(-c2ccccc2-c2ccccc2-c2ccccc2N(c2ccc(-c3cccc4c3sc3c(-c5ccccc5)cccc34)cc2)c2ccc3c(c2)C2(CC4CCC2C4)c2ccccc2-3)cc1]

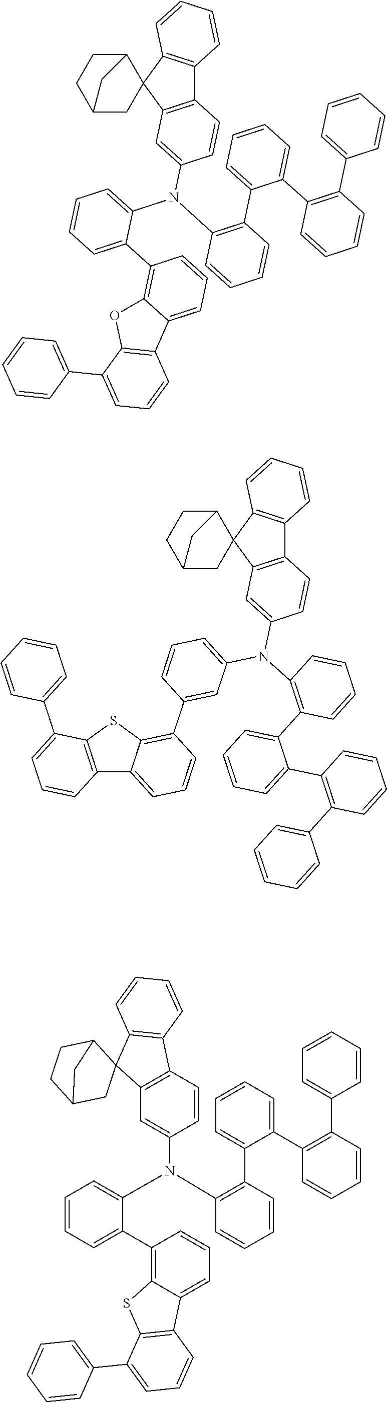 [SMILES: c1ccc(-c2ccccc2-c2ccccc2-c2ccccc2N(c2ccc3c(c2)C2(CC4CCC2C4)c2ccccc2-3)c2ccccc2-c2cccc3c2oc2c(-c4ccccc4)cccc23)cc1 ; c1ccc(-c2ccccc2-c2ccccc2-c2ccccc2N(c2cccc(-c3cccc4c3sc3c(-c5ccccc5)cccc34)c2)c2ccc3c(c2)C2(CC4CCC2C4)c2ccccc2-3)cc1 ; c1ccc(-c2ccccc2-c2ccccc2-c2ccccc2N(c2ccc3c(c2)C2(CC4CCC2C4)c2ccccc2-3)c2ccccc2-c2cccc3c2sc2c(-c4ccccc4)cccc23)cc1]

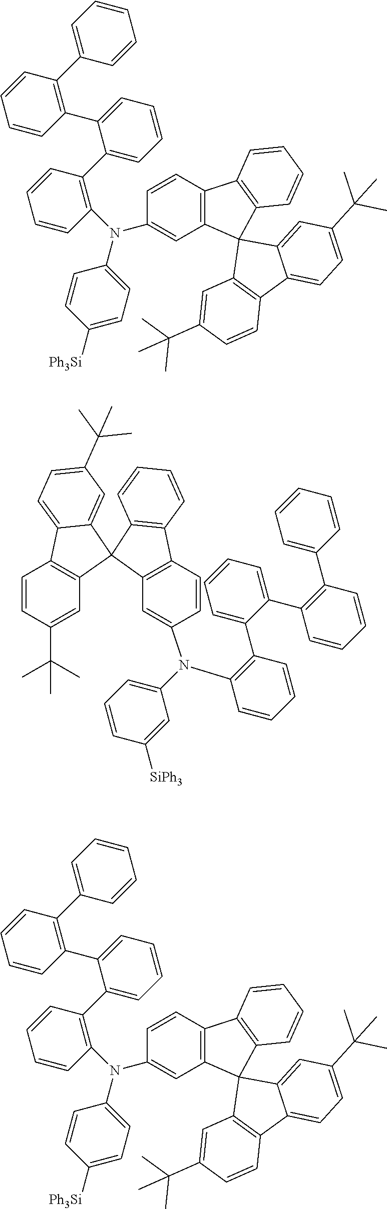

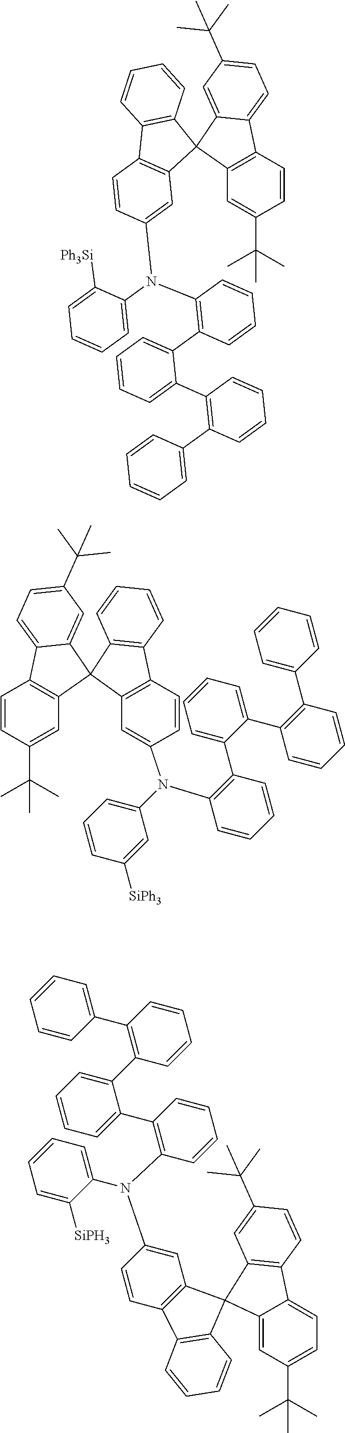

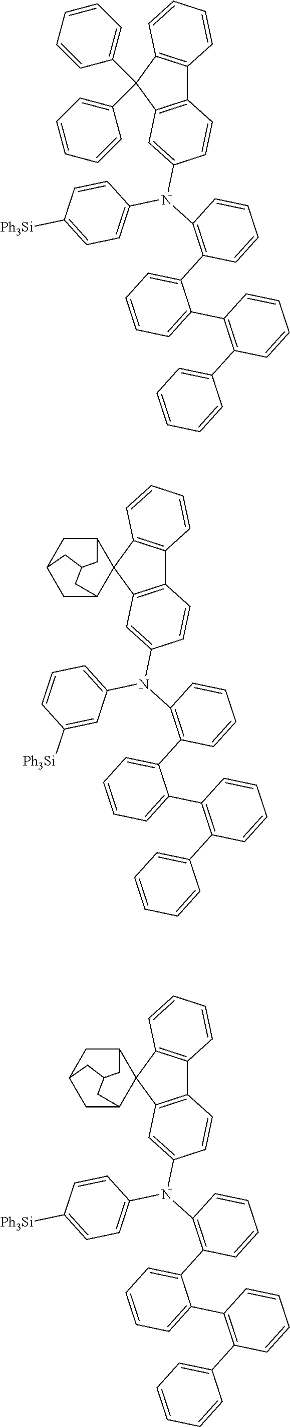

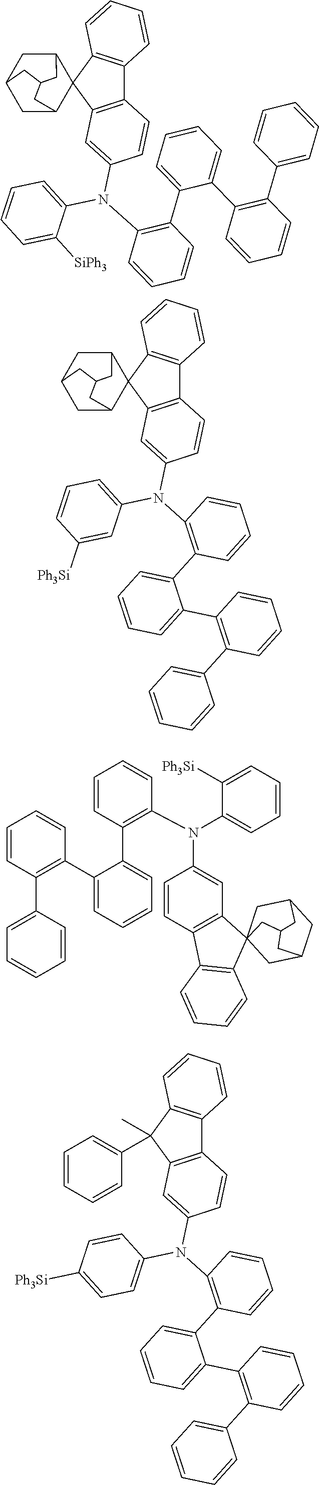

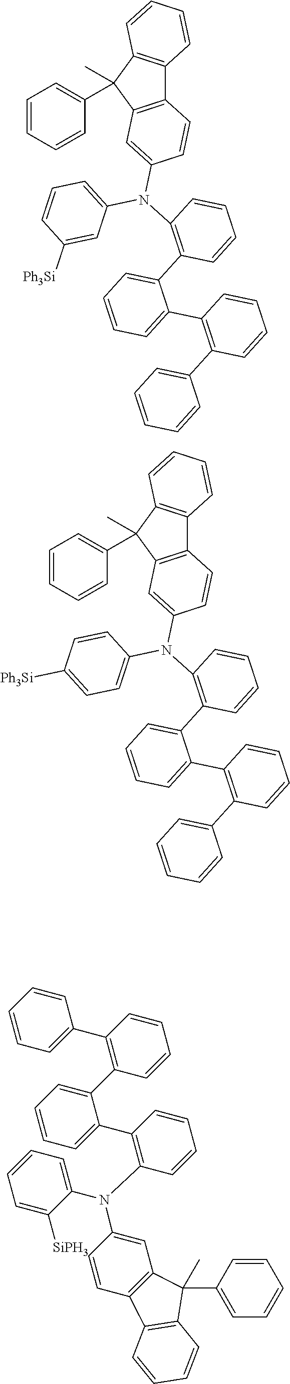

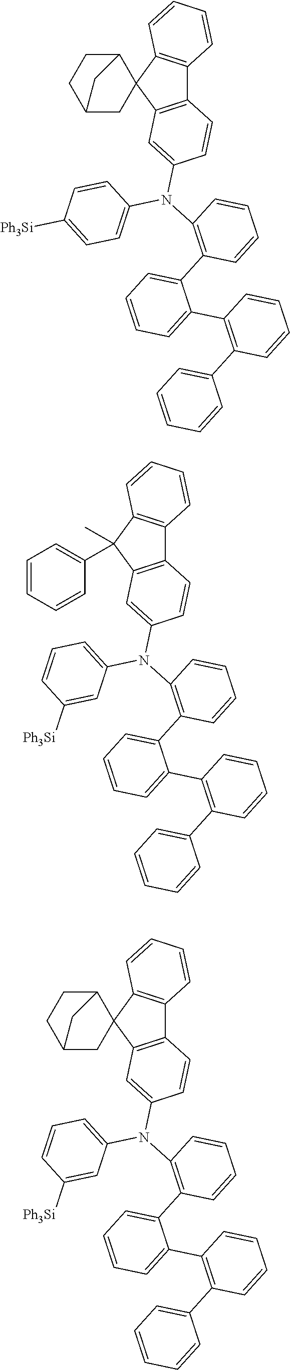

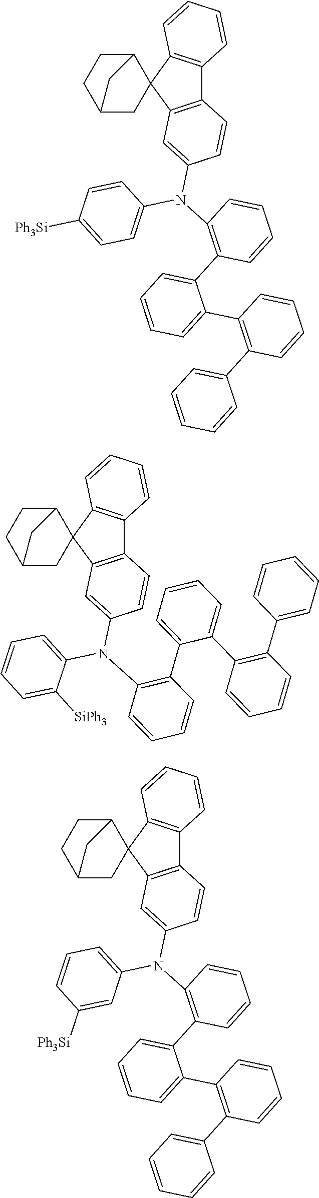

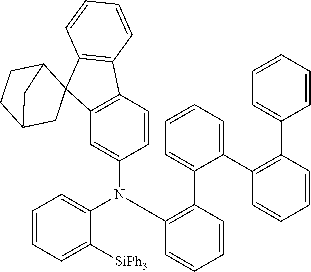

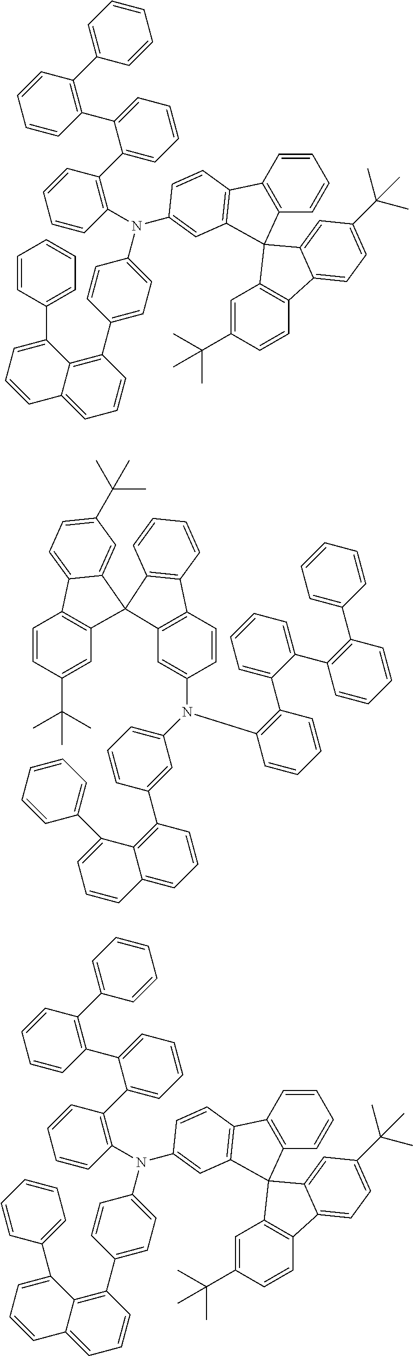

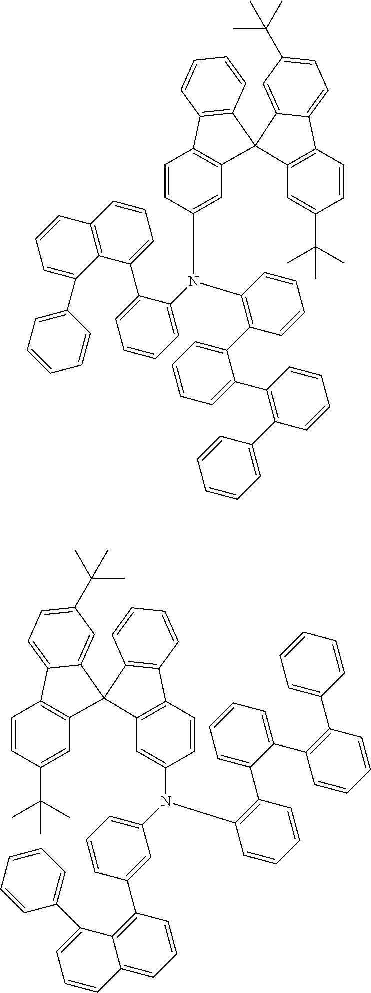 [SMILES: CC(C)(C)c1ccc2c(c1)C1(c3ccccc3-c3ccc(N(c4ccccc4-c4ccccc4-c4ccccc4-c4ccccc4)c4ccccc4-c4cccc5cccc(-c6ccccc6)c45)cc31)c1cc(C(C)(C)C)ccc1-2 ; CC(C)(C)c1ccc2c(c1)C1(c3ccccc3-c3ccc(N(c4cccc(-c5cccc6cccc(-c7ccccc7)c56)c4)c4ccccc4-c4ccccc4-c4ccccc4-c4ccccc4)cc31)c1cc(C(C)(C)C)ccc1-2]

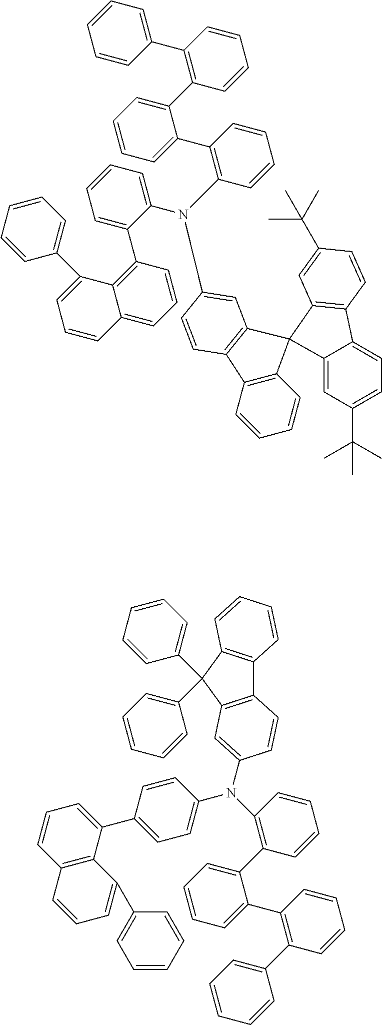 [SMILES: CC(C)(C)c1ccc2c(c1)C1(c3ccccc3-c3ccc(N(c4ccccc4-c4ccccc4-c4ccccc4-c4ccccc4)c4ccccc4-c4cccc5cccc(-c6ccccc6)c45)cc31)c1cc(C(C)(C)C)ccc1-2 ; c1ccc(-c2ccccc2-c2ccccc2-c2ccccc2N(c2ccc(-c3cccc4cccc(-c5ccccc5)c34)cc2)c2ccc3c(c2)C(c2ccccc2)(c2ccccc2)c2ccccc2-3)cc1]

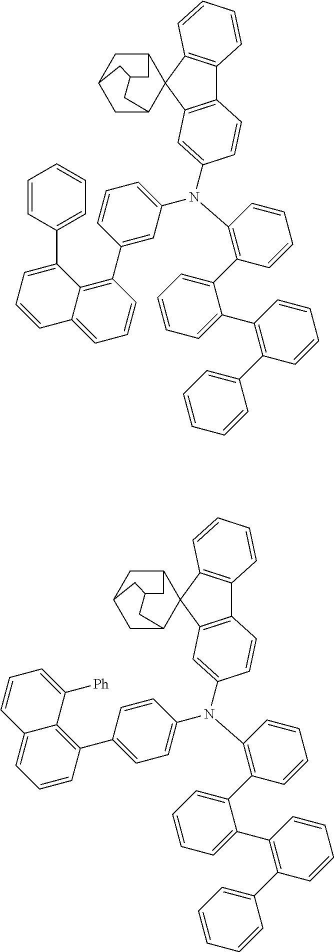 [SMILES: c1ccc(-c2ccccc2-c2ccccc2-c2ccccc2N(c2cccc(-c3cccc4cccc(-c5ccccc5)c34)c2)c2ccc3c(c2)C2(c4ccccc4-3)C3CC4CC(C3)CC2C4)cc1 ; c1ccc(-c2ccccc2-c2ccccc2-c2ccccc2N(c2ccc(-c3cccc4cccc(-c5ccccc5)c34)cc2)c2ccc3c(c2)C2(c4ccccc4-3)C3CC4CC(C3)CC2C4)cc1]

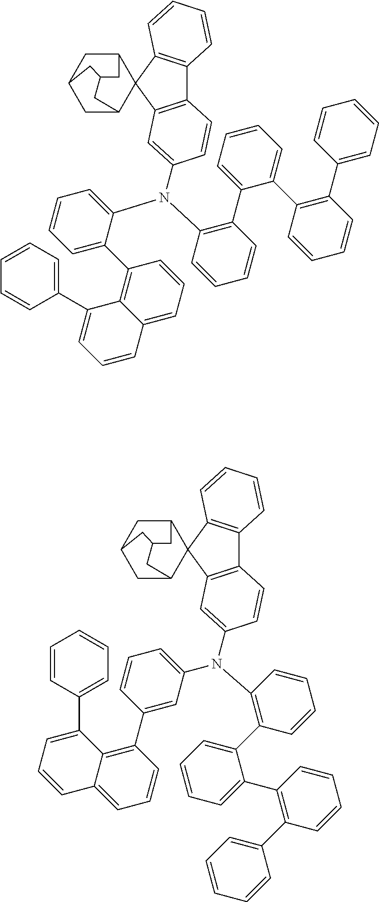 [SMILES: c1ccc(-c2ccccc2-c2ccccc2-c2ccccc2N(c2ccc3c(c2)C2(c4ccccc4-3)C3CC4CC(C3)CC2C4)c2ccccc2-c2cccc3cccc(-c4ccccc4)c23)cc1 ; c1ccc(-c2ccccc2-c2ccccc2-c2ccccc2N(c2cccc(-c3cccc4cccc(-c5ccccc5)c34)c2)c2ccc3c(c2)C2(c4ccccc4-3)C3CC4CC(C3)CC2C4)cc1]

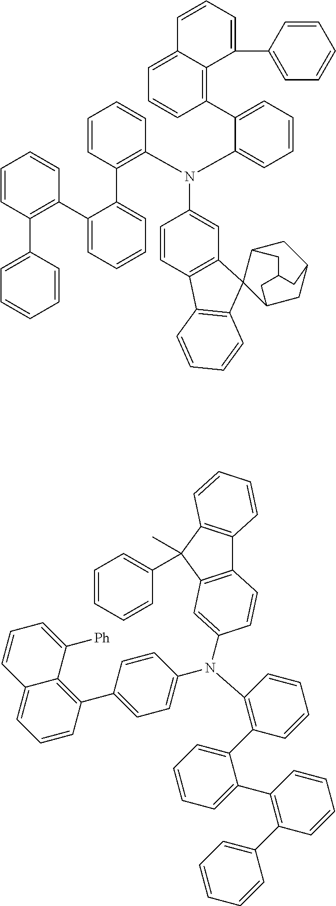 [SMILES: c1ccc(-c2ccccc2-c2ccccc2-c2ccccc2N(c2ccc3c(c2)C2(c4ccccc4-3)C3CC4CC(C3)CC2C4)c2ccccc2-c2cccc3cccc(-c4ccccc4)c23)cc1 ; CC1(c2ccccc2)c2ccccc2-c2ccc(N(c3ccc(-c4cccc5cccc(-c6ccccc6)c45)cc3)c3ccccc3-c3ccccc3-c3ccccc3-c3ccccc3)cc21]

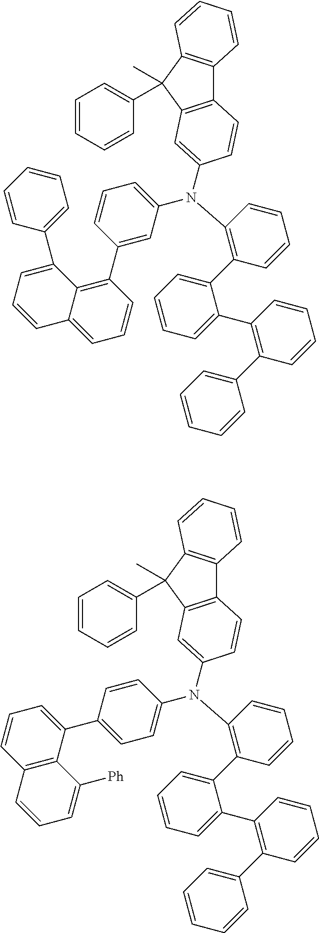 [SMILES: CC1(c2ccccc2)c2ccccc2-c2ccc(N(c3cccc(-c4cccc5cccc(-c6ccccc6)c45)c3)c3ccccc3-c3ccccc3-c3ccccc3-c3ccccc3)cc21 ; CC1(c2ccccc2)c2ccccc2-c2ccc(N(c3ccc(-c4cccc5cccc(-c6ccccc6)c45)cc3)c3ccccc3-c3ccccc3-c3ccccc3-c3ccccc3)cc21]

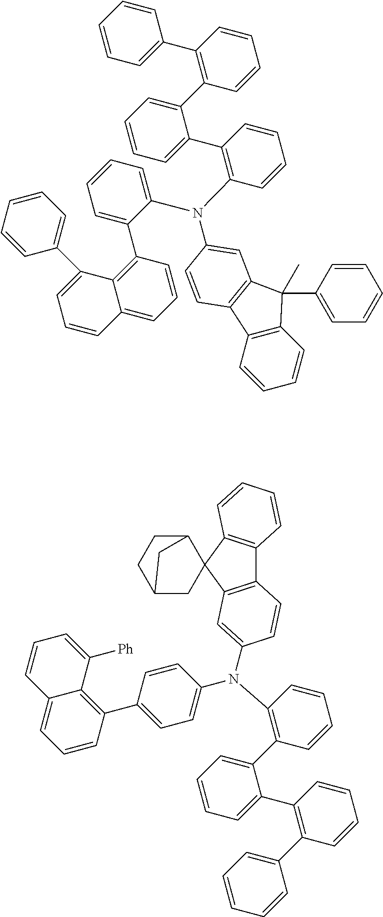 [SMILES: CC1(c2ccccc2)c2ccccc2-c2ccc(N(c3ccccc3-c3ccccc3-c3ccccc3-c3ccccc3)c3ccccc3-c3cccc4cccc(-c5ccccc5)c34)cc21 ; c1ccc(-c2ccccc2-c2ccccc2-c2ccccc2N(c2ccc(-c3cccc4cccc(-c5ccccc5)c34)cc2)c2ccc3c(c2)C2(CC4CCC2C4)c2ccccc2-3)cc1]

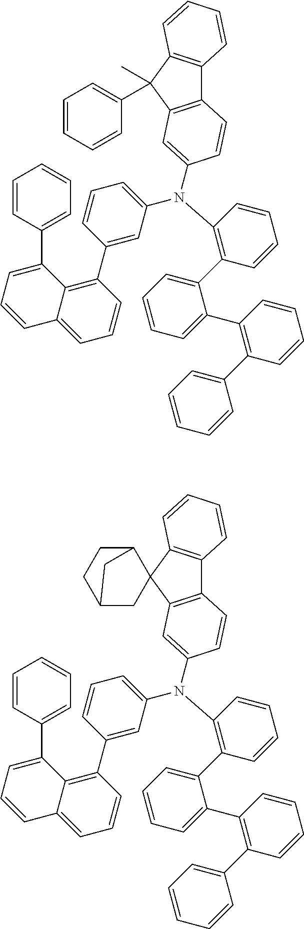 [SMILES: CC1(c2ccccc2)c2ccccc2-c2ccc(N(c3cccc(-c4cccc5cccc(-c6ccccc6)c45)c3)c3ccccc3-c3ccccc3-c3ccccc3-c3ccccc3)cc21 ; c1ccc(-c2ccccc2-c2ccccc2-c2ccccc2N(c2cccc(-c3cccc4cccc(-c5ccccc5)c34)c2)c2ccc3c(c2)C2(CC4CCC2C4)c2ccccc2-3)cc1]

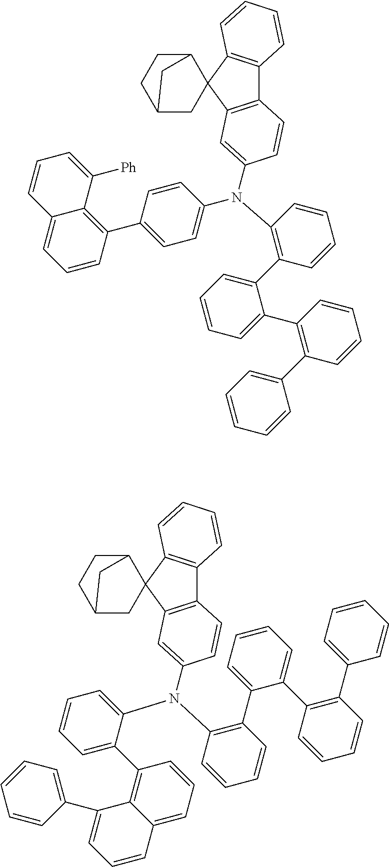 [SMILES: c1ccc(-c2ccccc2-c2ccccc2-c2ccccc2N(c2ccc(-c3cccc4cccc(-c5ccccc5)c34)cc2)c2ccc3c(c2)C2(CC4CCC2C4)c2ccccc2-3)cc1 ; c1ccc(-c2ccccc2-c2ccccc2-c2ccccc2N(c2ccc3c(c2)C2(CC4CCC2C4)c2ccccc2-3)c2ccccc2-c2cccc3cccc(-c4ccccc4)c23)cc1]

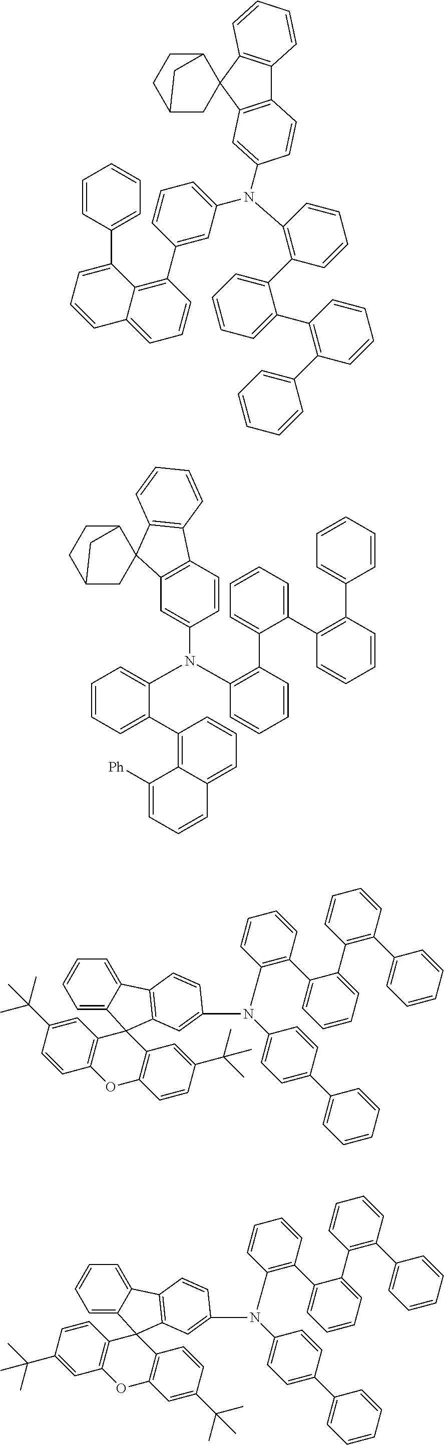 [SMILES: c1ccc(-c2ccccc2-c2ccccc2-c2ccccc2N(c2cccc(-c3cccc4cccc(-c5ccccc5)c34)c2)c2ccc3c(c2)C2(CC4CCC2C4)c2ccccc2-3)cc1 ; c1ccc(-c2ccccc2-c2ccccc2-c2ccccc2N(c2ccc3c(c2)C2(CC4CCC2C4)c2ccccc2-3)c2ccccc2-c2cccc3cccc(-c4ccccc4)c23)cc1 ; CC(C)(C)c1ccc2c(c1)C1(c3cc(C(C)(C)C)ccc3O2)c2ccccc2-c2ccc(N(c3ccc(-c4ccccc4)cc3)c3ccccc3-c3ccccc3-c3ccccc3-c3ccccc3)cc21 ; CC(C)(C)c1ccc2c(c1)Oc1cc(C(C)(C)C)ccc1C21c2ccccc2-c2ccc(N(c3ccc(-c4ccccc4)cc3)c3ccccc3-c3ccccc3-c3ccccc3-c3ccccc3)cc21]

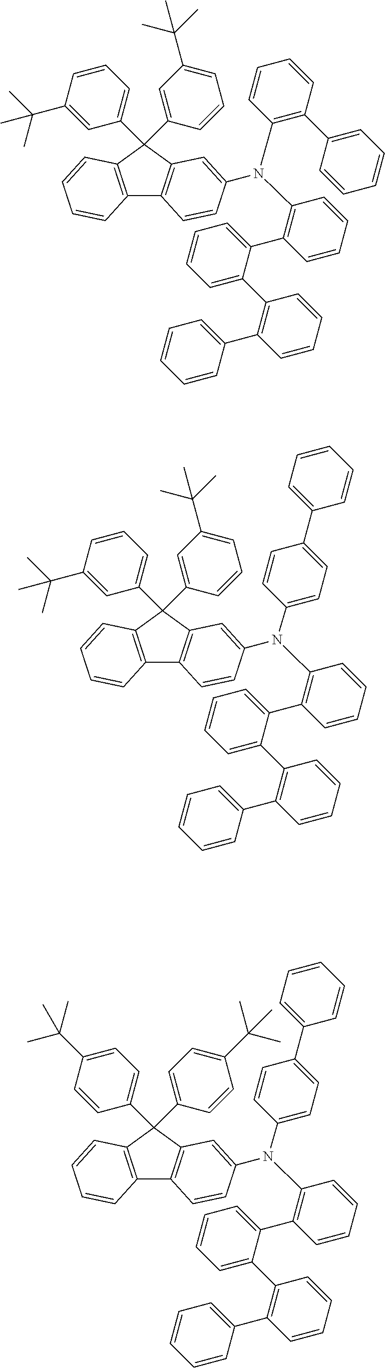 [SMILES: CC(C)(C)c1cccc(C2(c3cccc(C(C)(C)C)c3)c3ccccc3-c3ccc(N(c4ccccc4-c4ccccc4)c4ccccc4-c4ccccc4-c4ccccc4-c4ccccc4)cc32)c1 ; CC(C)(C)c1cccc(C2(c3cccc(C(C)(C)C)c3)c3ccccc3-c3ccc(N(c4ccc(-c5ccccc5)cc4)c4ccccc4-c4ccccc4-c4ccccc4-c4ccccc4)cc32)c1 ; CC(C)(C)c1ccc(C2(c3ccc(C(C)(C)C)cc3)c3ccccc3-c3ccc(N(c4ccc(-c5ccccc5)cc4)c4ccccc4-c4ccccc4-c4ccccc4-c4ccccc4)cc32)cc1]

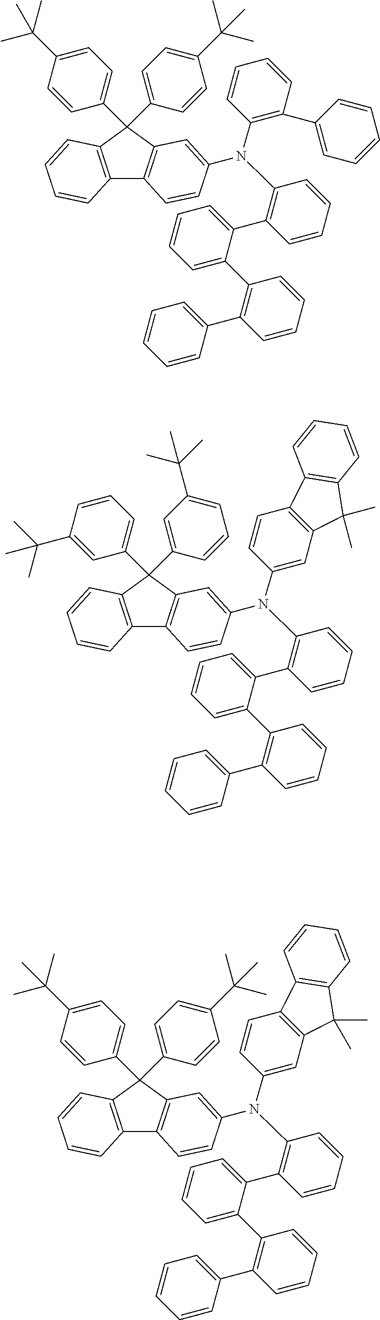

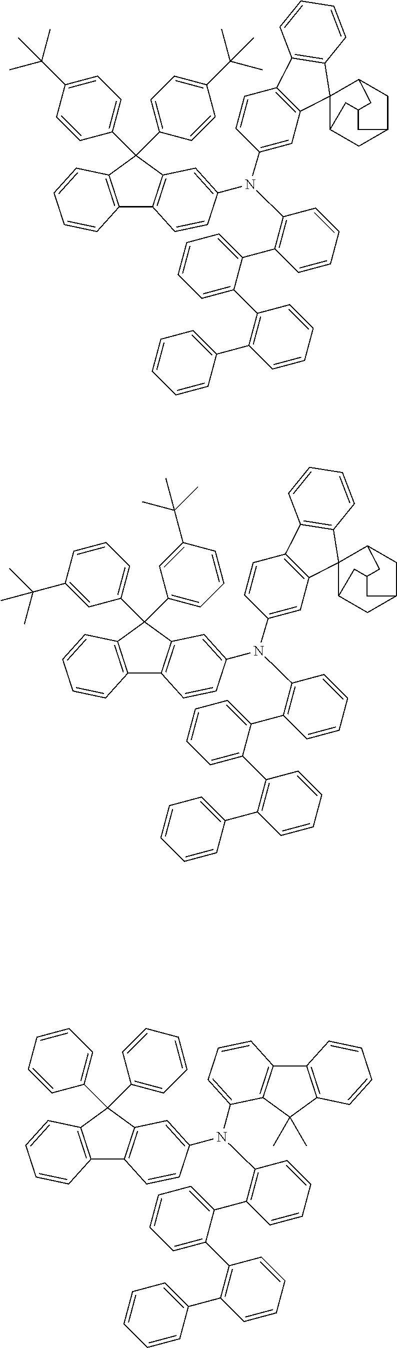 [SMILES: CC(C)(C)c1ccc(C2(c3ccc(C(C)(C)C)cc3)c3ccccc3-c3ccc(N(c4ccc5c(c4)C4(c6ccccc6-5)C5CC6CC(C5)CC4C6)c4ccccc4-c4ccccc4-c4ccccc4-c4ccccc4)cc32)cc1 ; CC(C)(C)c1cccc(C2(c3cccc(C(C)(C)C)c3)c3ccccc3-c3ccc(N(c4ccc5c(c4)C4(c6ccccc6-5)C5CC6CC(C5)CC4C6)c4ccccc4-c4ccccc4-c4ccccc4-c4ccccc4)cc32)c1 ; CC1(C)c2ccccc2-c2cccc(N(c3ccc4c(c3)C(c3ccccc3)(c3ccccc3)c3ccccc3-4)c3ccccc3-c3ccccc3-c3ccccc3-c3ccccc3)c21]

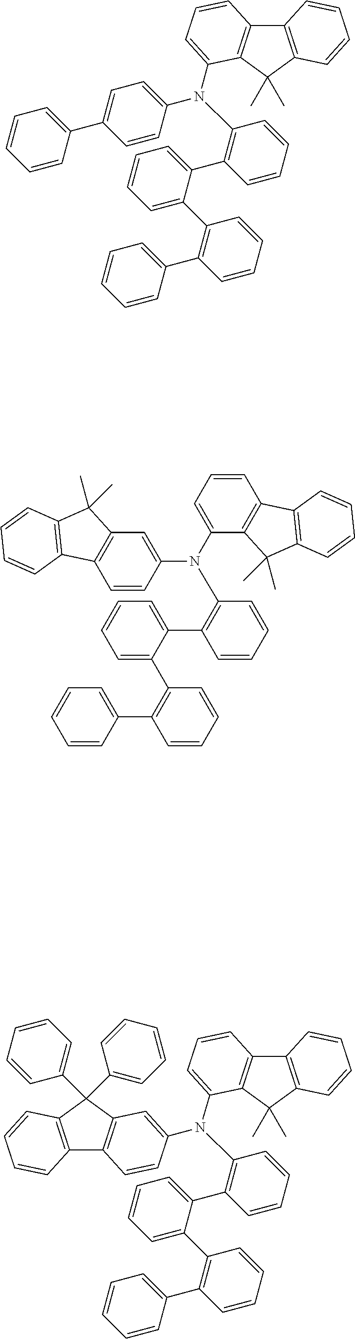 [SMILES: CC1(C)c2ccccc2-c2cccc(N(c3ccc(-c4ccccc4)cc3)c3ccccc3-c3ccccc3-c3ccccc3-c3ccccc3)c21 ; CC1(C)c2ccccc2-c2ccc(N(c3ccccc3-c3ccccc3-c3ccccc3-c3ccccc3)c3cccc4c3C(C)(C)c3ccccc3-4)cc21 ; CC1(C)c2ccccc2-c2cccc(N(c3ccc4c(c3)C(c3ccccc3)(c3ccccc3)c3ccccc3-4)c3ccccc3-c3ccccc3-c3ccccc3-c3ccccc3)c21]

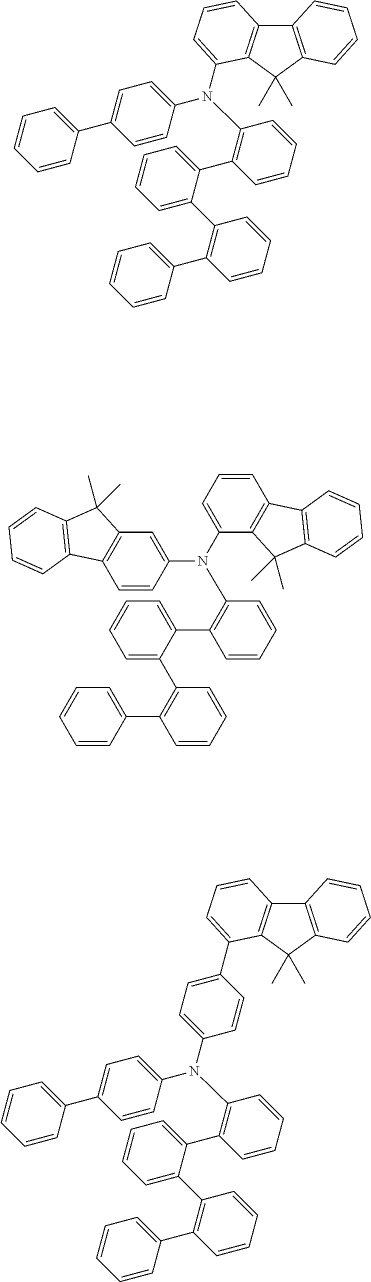 [SMILES: CC1(C)c2ccccc2-c2cccc(N(c3ccc(-c4ccccc4)cc3)c3ccccc3-c3ccccc3-c3ccccc3-c3ccccc3)c21 ; CC1(C)c2ccccc2-c2ccc(N(c3ccccc3-c3ccccc3-c3ccccc3-c3ccccc3)c3cccc4c3C(C)(C)c3ccccc3-4)cc21 ; CC1(C)c2ccccc2-c2cccc(-c3ccc(N(c4ccc(-c5ccccc5)cc4)c4ccccc4-c4ccccc4-c4ccccc4-c4ccccc4)cc3)c21]

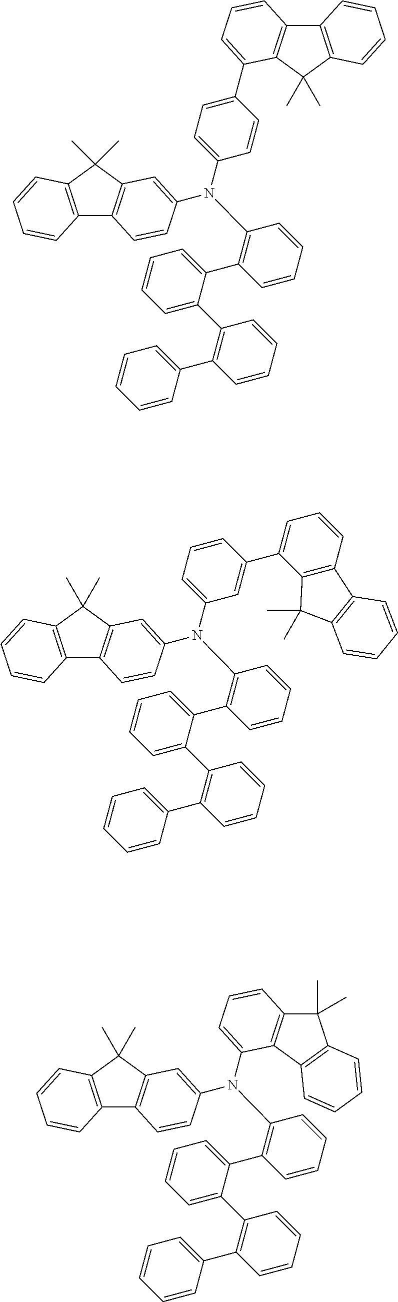 [SMILES: CC1(C)c2ccccc2-c2ccc(N(c3ccc(-c4cccc5c4C(C)(C)c4ccccc4-5)cc3)c3ccccc3-c3ccccc3-c3ccccc3-c3ccccc3)cc21 ; CC1(C)c2ccccc2-c2ccc(N(c3cccc(-c4cccc5c4C(C)(C)c4ccccc4-5)c3)c3ccccc3-c3ccccc3-c3ccccc3-c3ccccc3)cc21 ; CC1(C)c2ccccc2-c2ccc(N(c3ccccc3-c3ccccc3-c3ccccc3-c3ccccc3)c3cccc4c3-c3ccccc3C4(C)C)cc21]

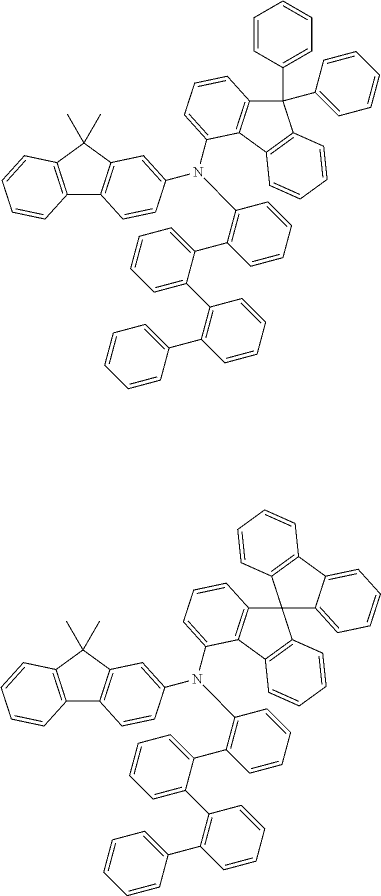 [SMILES: CC1(C)c2ccccc2-c2ccc(N(c3ccccc3-c3ccccc3-c3ccccc3-c3ccccc3)c3cccc4c3-c3ccccc3C4(c3ccccc3)c3ccccc3)cc21 ; CC1(C)c2ccccc2-c2ccc(N(c3ccccc3-c3ccccc3-c3ccccc3-c3ccccc3)c3cccc4c3-c3ccccc3C43c4ccccc4-c4ccccc43)cc21]

### Material for Organic EL Device

The material for organic EL device of the present invention contains an inventive compound. The content of the inventive compound in the material for organic EL device is 1 mass % or more (including 100%), preferably 10 mass % or more (including 100%), more preferably 50 mass % or more (including 100%), further preferably 80 mass % or more (including 100%), particularly preferably 90 mass % or more (including 100%). The material for organic EL device of the present invention is useful for manufacturing an organic EL device.

### Organic EL Device

An organic EL device which is one embodiment of the present invention contains an anode, a cathode, and an organic layer placed between the anode and the cathode. The organic layer contains a light emitting layer, and at least one layer of the organic layer contains an inventive compound.

Examples of the organic layer containing the inventive compound include a hole transporting band (hole injecting layer, hole transporting layer, electron blocking layer, exciton blocking layer, etc.) provided between the anode and the light emitting layer, the light emitting layer, a space layer, an electron transport zone (electron injecting layer, electron transporting layer, hole blocking layer, etc.) provided between the cathode and the light emitting layer, however it is not limited thereto. The inventive compound is preferably used as a material for a hole transporting band or a light emitting layer of a fluorescent or phosphorescent EL device, more preferably used as a material for a hole transport zone, and further more preferably used as a material for a hole injecting layer, a hole transporting layer, an electron blocking layer, or an exciton blocking layer, particularly preferably used as a material for a hole injecting layer or a hole transporting layer.

The organic EL device of the present invention may be a monochromatic fluorescent or phosphorescent type light emitting element, or a fluorescent/phosphorescent hybrid type white light emitting element, and may be a simple type having a single light emitting unit, or a tandem type having plural light emitting units, and it is preferably a fluorescent type light emitting element. Here, the “light emitting unit” refers to a minimum unit which contains an organic layer, in which at least one of the layers is a light emitting layer, and emits light by recombining injected holes and electrons.

For example, examples of the typical element configuration of a simple type organic EL device include the following element configurations.

### (1) Anode/Light Emitting Unit/Cathode

Further, the light emitting unit may be a multilayer type having plural phosphorescent light emitting layers or fluorescent light emitting layers, and in that case, a space layer may be contained between each light emitting layer, for the purpose of preventing excitons which are generated in the phosphorescent light emitting layer from diffusing into the fluorescent light emitting layer. A typical layer configuration of a simple type light emitting unit is shown below: Layers in parentheses are optional.
(a) (Hole injecting layer/) Hole transporting layer/Fluorescent light emitting layer/Electron transporting layer (/Electron injecting layer)
(b) (Hole injecting layer/) Hole transporting layer/First fluorescent light emitting layer/Second fluorescent light emitting layer/Electron transporting layer (/Electron injecting layer)
(c) (Hole injecting layer/) Hole transporting layer/Phosphorescent light emitting layer/Space layer/Fluorescent light emitting layer/Electron transporting layer (/Electron injecting layer)
(d) (Hole injecting layer/) Hole transporting layer/First phosphorescent light emitting layer/Second phosphorescent light emitting layer/Space layer/Fluorescent light emitting layer/Electron transporting layer (/Electron injecting layer)
(e) (Hole injecting layer/) Hole transporting layer/Phosphorescent light emitting layer/Space layer/First fluorescent light emitting layer/Second fluorescent light emitting layer/Electron transporting layer (/Electron injecting layer)
(f) (Hole injecting layer/) Hole transporting layer/Electron blocking layer/Fluorescent light emitting layer/Electron transporting layer (/Electron injecting layer)
(g) (Hole injecting layer/) Hole transporting layer/Exciton blocking layer/Fluorescent light emitting layer/Electron transporting layer (/Electron injecting layer)
(h) (Hole injecting layer/) First hole transporting layer/Second hole transporting layer/Fluorescent light emitting layer/Electron transporting layer (/Electron injecting layer)
(i) (Hole injecting layer/) First hole transporting layer/Second hole transporting layer/Fluorescent light emitting layer/First electron transporting layer/Second electron transporting layer (/Electron injecting layer)
(j) (Hole injecting layer/) Hole transporting layer/Fluorescent light emitting layer/Hole blocking layer/Electron transporting layer (/Electron injecting layer)
(k) (Hole injecting layer/) Hole transporting layer/Fluorescent light emitting layer/Exciton blocking layer/Electron transporting layer (/Electron injecting layer)
(l) (Hole injecting layer/) First hole transporting layer/Second hole transporting layer/First fluorescent light emitting layer/Second fluorescent light emitting layer/First electron transporting layer/Second electron transporting layer (/Electron injecting layer)
(m) (Hole injecting layer/) First hole transporting layer/Second hole transporting layer/Third hole transporting layer/First fluorescent light emitting layer/Second fluorescent light emitting layer/First electron transporting layer/Second electron transporting layer (/Electron injecting layer)
(n) (Hole injecting layer/) First hole transporting layer/Second hole transporting layer/Third hole transporting layer/Fluorescent light emitting layer/First electron transporting layer/Second electron transporting layer (/Electron injecting layer)

Each of the phosphorescent or fluorescent light emitting layers may respectively show different light emitting colors. Specifically, in the light emitting unit (f), examples include a layer configuration of (Hole injecting layer/) Hole transporting layer/First phosphorescent light emitting layer (red light emitting)/Second phosphorescent light emitting layer (green light emitting)/Space layer/Fluorescent light emitting layer (blue light emitting)/Electron transporting layer, etc.

Note that an electron blocking layer may be provided between each light emitting layer and the hole transporting layer or space layer, as appropriate. Further, a hole blocking layer may be provided between each light emitting layer and the electron transporting layer as appropriate. By providing an electron blocking layer or a hole blocking layer, it is possible to confine electrons or holes within the light emitting layer, increase the probability of charge recombination in the light emitting layer, and improve a light emission efficiency.

Examples of the typical device configuration of tandem type organic EL device include the following element configurations.

### (2) Anode/First Light Emitting Unit/Intermediate Layer/Second Light Emitting Unit/Cathode

Here, for example, as the first light emitting unit and the second light emitting unit, each independently can be selected from the above light emitting units.

The intermediate layer is generally also referred to as an intermediate electrode, intermediate conductive layer, charge generation layer, electron extraction layer, connection layer, or intermediate insulating layer, and any known material configuration in which electrons are provided to the first light emitting unit and holes are provided to the second light emitting unit, can be used.

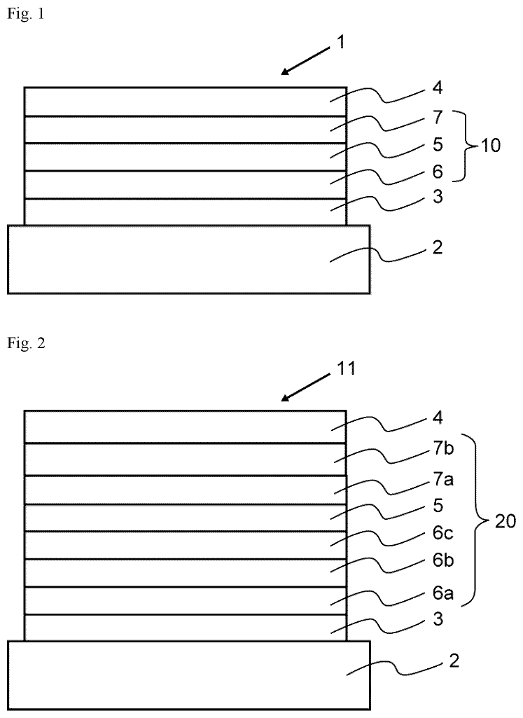

FIG. 1 is a schematic diagram of an example of the configuration of an organic EL device of the present invention. An organic EL device 1 contains a substrate 2, an anode 3, a cathode 4, and a light emitting unit 10 placed between the anode 3 and the cathode 4. The light emitting unit 10 contains a light emitting layer 5. A hole transporting band 6 (hole injecting layer, hole transporting layer, etc.) is contained between the light emitting layer 5 and the anode 3, and an electron transporting band 7 (electron injecting layer, electron transporting layer, etc.) is contained between the light emitting layer 5 and the cathode 4. Further, an electron blocking layer (not shown) may be provided on the anode 3 side of the light emitting layer 5, and a hole blocking layer (not shown) may be provided on the cathode 4 side of the light emitting layer 5. Thereby, electrons and holes can be confined in the light-emitting layer 5, and the efficiency of exciton generation in the light emitting layer 5 can be further increased.

FIG. 2 is a schematic diagram of other configuration of an organic EL device of the present invention. An organic EL device 11 contains a substrate 2, an anode 3, a cathode 4, and a light emitting unit 20 placed between the anode 3 and the cathode 4. The light emitting unit 20 contains a light emitting layer 5. The hole transporting band placed between the anode 3 and the light emitting layer 5 is formed from a hole injecting layer 6a, a first hole transporting layer 6b, and a second hole transporting layer 6c. The electron transporting band placed between the light emitting layer 5 and the cathode 4 is formed from a first electron transporting layer 7a and a second electron transporting layer 7b.

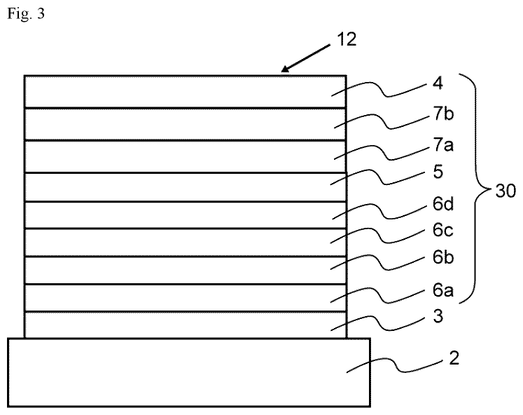

FIG. 3 is a schematic diagram of other configuration of an organic EL device of the present invention. An organic EL device 12 contains a substrate 2, an anode 3, a cathode 4, and a light emitting unit 30 placed between the anode 3 and the cathode 4. The light emitting unit 30 contains a light emitting layer 5. The hole transporting band placed between the anode 3 and the light emitting layer 5 is formed from a hole injecting layer 6a, a first hole transporting layer 6b, a second hole transporting layer 6c, and a third hole transporting layer 6d. The electron transporting band placed between the light emitting layer 5 and the cathode 4 is formed from a first electron transporting layer 7a and a second electron transporting layer 7b.

In the present invention, a host combined with a fluorescent dopant material (fluorescent material) is referred to as a fluorescent host, and a host combined with a phosphorescent dopant material is referred to as a phosphorescent host. Fluorescent hosts and phosphorescent hosts are not distinguished only by molecular structure. That is, the phosphorescent host means a material forming a phosphorescent light emitting layer containing a phosphorescent dopant, and does not mean that it cannot be used as a material to form a fluorescent light emitting layer. The same applies to fluorescent hosts.

### Substrate

The substrate is used as a support for the organic EL device. As the substrate, for example, a plate of glass, quartz, and plastic can be used. Further, a flexible substrate may be used. Examples of the flexible substrate include plastic substrates made of polycarbonate, polyarylate, polyethersulfone, polypropylene, polyester, polyvinyl fluoride, and polyvinyl chloride. Moreover, an inorganic vapor-deposited film can also be used.

### Anode

For the anode formed on the substrate, it is preferable to use a metal, an alloy, an electrically conductive compound, a mixture thereof, or the like having a large work function (specifically, 4.0 eV or more). Specifically, for example, examples include indium oxide-tin oxide (ITO), indium oxide-tin oxide containing silicon or silicon oxide, indium oxide-zinc oxide, indium oxide containing tungsten oxide and zinc oxide, and graphene. In addition, examples include gold (Au), platinum (Pt), nickel (Ni), tungsten (W), chromium (Cr), molybdenum (Mo), iron (Fe), cobalt (Co), copper (Cu), palladium (Pd), titanium (Ti), or nitrides of the above metals (for example, titanium nitride).

These materials are usually film-formed by sputtering method. For example, the indium oxide-zinc oxide can be formed by using a target in which 1 to 10 wt % of zinc oxide is added to indium oxide, and the indium oxide containing tungsten oxide and zinc oxide can be formed by using a target in which 0.5 to 5 wt % of tungsten oxide and 0.1 to 1 wt % of zinc oxide are contained into indium oxide, with a sputtering method. In addition, it may be produced by a vacuum deposition method, a coating method, an inkjet method, a spin coating method, or the like.

### Hole Transporting Band

As mentioned above, the organic layer may contain a hole transporting band between the anode and the light emitting layer. The hole transporting band is composed of a hole injecting layer, a hole transporting layer, an electron blocking layer, and the like. The Hole transporting band preferably contains the inventive compound. At least one of these layers constituting the hole transporting layer preferably contains the inventive compound, and particularly the inventive compound is more preferably contained in the hole transporting layer.

Since the hole injecting layer formed in contact with the anode is formed using a material which facilitate hole injection regardless of the work function of the anode, materials commonly used as electrode materials (for example, metals, alloys, electrically conductive compounds, mixtures thereof, elements belonging to Group 1 or Group 2 of the periodic table of elements) can be used.

Elements belonging to Group 1 or Group 2 of the Periodic Table of Elements, which are materials with a small work function, that is, alkali metals such as lithium (Li) and cesium (Cs), and alkaline earth metals such as magnesium (Mg), calcium (Ca), and strontium (Sr), alloys containing these (for example, MgAg, AlLi), rare earth metals such as europium (Eu), ytterbium (Yb), alloys containing these, can also be used. In addition, when forming an anode using an alkali metal, an alkaline earth metal, or an alloy containing these, a vacuum deposition method or a sputtering method can be used. Furthermore, when silver paste or the like is used, a coating method, an inkjet method, or the like can be used.

### Hole Injecting Layer

The hole injecting layer is a layer containing a material having high hole injection properties (hole injecting material), and is formed between the anode and the light emitting layer, or, between the hole transporting layer and the anode when present.

As the hole injecting materials other than the inventive compound, molybdenum oxide, titanium oxide, vanadium oxide, rhenium oxide, ruthenium oxide, chromium oxide, zirconium oxide, hafnium oxide, tantalum oxide, silver oxide, tungsten oxide, manganese oxide, etc. can be used.

Examples of the hole injecting materials contain aromatic amine compounds such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), 4,4′-bis(N-{4-[N′-(3-methylphenyl)-N′-phenylamino]phenyl}-N-phenylamino) biphenyl (abbreviation: DNTPD), 1,3,5-tris[N-(4-diphenylaminophenyl)-N-phenylamino]benzene (abbreviation: DPA3B), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), 3-[N-(1-naphthyl)-N-(9-phenylcarbazol-3-yl)amino]-9-phenylcarbazole (abbreviation: PCzPCN1), which are low molecular weight organic compounds.

High molecular weight compounds (oligomers, dendrimers, polymers, etc.) can also be used. For example, examples include high molecular weight compounds such as poly (N-vinylcarbazole) (abbreviation: PVK), poly(4-vinyltriphenylamine) (abbreviation: PVTPA), poly[N-(4-{N′-[4-(4-diphenylamino)phenyl]phenyl-N′-phenylamino}phenyl) methacrylamide] (abbreviation: PTPDMA), poly[N,N′-bis(4-butylphenyl)-N,N′-bis(phenyl)benzidine] (abbreviation: Poly-TPD). In addition, high molecular weight compounds to which an acid is added, such as poly(3,4-ethylenedioxy thiophene)/poly(styrene sulfonic acid) (PEDOT/PSS) or polyaniline/poly(styrene sulfonic acid) (PAni/PSS), can also be used.

Furthermore, it is also preferable to use acceptor materials such as a hexaazatriphenylene (HAT) compound represented by the following formula (K).

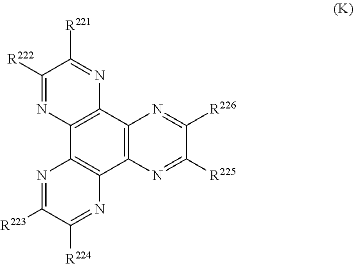

(In the above formula, R²²¹ to R²²⁶ each independently represent a cyano group, —CONH₂, a carboxy group, or —COOR²²⁷ (R²²⁷ represents an alkyl group having 1 to 20 carbon atoms or a cycloalkyl group having 3 to 20 carbon atoms). Furthermore, two adjacent groups selected from R²²¹ and R²²², R²²³ and R²²⁴, and R²²⁵ and R²²⁶ may be bonded to each other to form a group represented by —CO—O—CO—).

Examples of R²²⁷ include a methyl group, an ethyl group, a n-propyl group, an isopropyl group, a n-butyl group, an isobutyl group, a t-butyl group, a cyclopentyl group, and a cyclohexyl group.

### Hole Transporting Layer

The hole transporting layer is a layer containing a material having high hole transport properties (hole transporting material), and is formed between the anode and the light emitting layer, or, between the hole injecting layer and the light emitting layer when present. The inventive compounds may be used alone or in combination with the compounds listed below in the hole transporting layer.

The hole transporting layer may have a single-layer structure or a multilayer structure including two or more layers. For example, the hole transporting layer may have a two-layer structure including a first hole transporting layer (on the anode side) and a second hole transporting layer (on the cathode side). That is, the hole transporting band may include the first hole transporting layer on the anode side and the second hole transporting layer on the cathode side. Further, the hole transporting layer may have a three-layer structure containing, in order from the anode side, a first hole transporting layer, a second hole transporting layer, and a third hole transporting layer. That is, the third hole transporting layer may be placed between the second hole transporting layer and the light emitting layer.

In one embodiment of the present invention, the hole transporting layer having the single-layer structure is preferably adjacent to the light emitting layer, and the hole transporting layer closest to the cathode in the multilayer structure which is, for example, the second hole transporting layer in the two-layer structure and the third hole transporting layer in the three-layer structure are preferably adjacent to the light emitting layer. In another embodiment of the present invention, an electron blocking layer, which will be described later, may be interposed between the hole transporting layer and the light emitting layer in the single-layer structure, or between the hole transporting layer closest to the light emitting layer and the light emitting layer in the multilayer structure.

When the hole transporting layer has a two-layer structure, at least one of the first hole transporting layer and the second hole transporting layer contains the inventive compound. That is, the inventive compound is contained only in the first hole transporting layer, only in the second hole transporting layer, or in both the first hole transporting layer and the second hole transporting layer.

In one embodiment of the present invention, the inventive compound is preferably included in the second hole transporting layer. That is, it is preferable that the inventive compound is contained only in the second hole transporting layer, or that the inventive compound is contained in the first hole transporting layer and the second hole transporting layer.

When the hole transporting layer has a three-layer structure, at least one of the first to third hole transporting layers contains the inventive compound. That is, the inventive compound is contained in only one layer selected from the first to third hole transporting layers (only the first hole transporting layer, only the second hole transporting layer, or only the third hole transporting layer), in only two layers selected from the first to third hole transporting layers (only the first hole transporting layer and the second hole transporting layer, only the first hole transporting layer and the third hole transporting layer, or only the second hole transporting layer), or in all of the first to third hole transporting layers.

In one embodiment of the present invention, the inventive compound is preferably included in the third hole transporting layer. That is, it is preferable that the inventive compound is contained only in the third hole transporting layer, or that the inventive compound is preferably contained in the third hole transporting layer and one or both of the first hole transporting layer and the second hole transporting layer.

In one embodiment of the present invention, the inventive compound contained in each of the hole transporting layers is preferably a light hydrogen body from the viewpoint of manufacturing cost. The light hydrogen body refers to an inventive compound in which all hydrogen atoms are light hydrogen atoms.

Therefore, the present invention includes an organic EL device in which one or both of the first hole transporting layer and the second hole transporting layer (in the case of a two-layer structure), or at least one of the first to third hole transporting layers contain an inventive compound which substantially contains only a light hydrogen body. The “inventive compound which substantially contains only a light hydrogen body” means that the content ratio of the light hydrogen body with respect to the total amount of inventive compound is 90 mol % or more, preferably 95 mol % or more, more preferably 99 mol % or more (including 100% for each).

As the hole transporting layer material other than the inventive compound, for example, aromatic amine compounds, carbazole derivatives, anthracene derivatives can be used.

Examples of aromatic amine compounds include 4,4′-bis[N-(1-naphthyl)-N-phenylamino]biphenyl (abbreviation: NPB), N,N′-bis(3-methylphenyl)-N,N′-diphenyl-[1,1′-biphenyl]-4,4′-diamine (abbreviation: TPD), 4-phenyl-4′-(9-phenylfluoren-9-yl)triphenylamine (abbreviation: BAFLP), 4,4′-bis[N-(9,9-dimethylfluoren-2-yl)-N-phenylamino]biphenyl (abbreviation: DFLDPBi), 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), and 4,4′-bis[N-(spiro-9,9′-bifluoren-2-yl)-N-phenylamino]biphenyl (abbreviation: BSPB). The above compounds have a hole mobility of 10⁻⁶ cm²/Vs or more.

Examples of carbazole derivatives include 4,4′-di(9-carbazolyl) biphenyl (abbreviation: CBP), 9-[4-(9-carbazolyl)phenyl]-10-phenylanthracene (abbreviation: CzPA), and 9-phenyl-3-[4-(10)-phenyl-9-anthryl)phenyl]-9H-carbazole (abbreviation: PCzPA).

Examples of anthracene derivatives include 2-t-butyl-9,10-di(2-naphthyl) anthracene (abbreviation: t-BuDNA), 9,10-di(2-naphthyl) anthracene (abbreviation: DNA), and 9,10-diphenylanthracene (abbreviation: DPAnth).

High molecular weight compound such as poly(N-vinylcarbazole) (abbreviation: PVK) and poly(4-vinyltriphenylamine) (abbreviation: PVTPA) can also be used.

Provided that compounds other than those mentioned above may be used as long as they have higher hole transport properties than electron transport properties.

In the organic EL device having a hole transporting layer having a two-layer structure according to the present invention, the first hole transporting layer preferably contains one or plural of the compounds represented by the following formula (11) or formula (12).

In the organic EL device having a hole transporting layer having three-layer structure according to the present invention, one or both of the first hole transporting layer and the second hole transporting layer preferably contains one or plurality of the compounds represented by the following formula (11) or formula (12).

In the organic EL device of the present invention having a hole transporting layer with an n-layer structure (n is an integer of 4 or more), at least one of the first to the (n−1)^{th} hole transporting layers preferably contains one or plural compounds represented by (11) or formula (12).

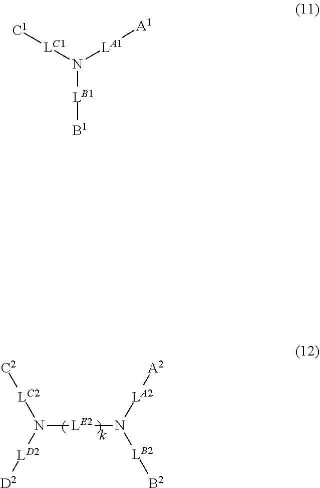

[In the formula (11) and (12),
L^{A1}, L^{B1}, L^{C1}, L^{A2}, L^{B2}, L^{C2} and L^{D2} are each independently a single bond, a substituted or unsubstituted arylene group having 6 to 50 ring carbon atoms, or a divalent heterocyclic group having 5 to 50 ring atoms,
k is 1, 2, 3 or 4,
when k is 1, L^{E2} is a substituted or unsubstituted arylene group having 6 to 50 ring carbon atoms, or a substituted or unsubstituted divalent heterocyclic group having 5 to 50 ring atoms,
when k is 2, 3 or 4, 2, 3 or 4 L^{E2}s are the same as or different from each other,
when k is 2, 3 or 4, the plural of L^{E2}s are bonded to each other to form a substituted or unsubstituted monocycle, bonded to each other to form a substituted or unsubstituted condensed ring, or are not bonded to each other,
in which the L^{E2} which does not form the single ring and does not form the condensed ring is a substituted or unsubstituted arylene group having 6 to 50 ring carbon atoms, or a substituted or unsubstituted divalent heterocyclic group having 5 to 50 ring atoms,
A¹, B¹, C¹, A², B², C², and D² are each independently a substituted or unsubstituted aryl group having 6 to 50 ring carbon atoms, a substituted or unsubstituted heterocyclic group having 5 to 50 ring atoms, or —Si(R′₉₀₁) (R′₉₀₂) (R′₉₀₃),
R′₉₀₁, R′₉₀₂ and R′₉₀₃ are each independently a substituted or unsubstituted aryl group having 6 to 50 ring carbon atoms,
when plural R′₉₀₁s exist, the plural R′₉₀₁s are the same as or different from each other,
when plural R′₉₀₂s exist, the plural R′₉₀₂s are the same as or different from each other,
when plural R′₉₀₃s exist, the plural R′₉₀₃s are the same as or different from each other.]

In formulas (11) and (12), A¹, B¹, C¹, A², B², C², and D² are preferably each independently selected from a substituted or unsubstituted phenylene group, a substituted or unsubstituted biphenyl group, a substituted or unsubstituted terphenyl group, a substituted or unsubstituted naphthyl group, a substituted or unsubstituted fluorenyl group, a substituted or unsubstituted dibenzofuranyl group, a substituted or unsubstituted dibenzothiophenyl group, and a substituted or unsubstituted carbazolyl group group.

Further, more preferably, in formula (11), at least one of A¹, B¹, and C¹, and in formula (12), at least one of A², B², C², and D² is a substituted or unsubstituted biphenyl group, a substituted or unsubstituted terphenyl group, a substituted or unsubstituted naphthyl group, a substituted or unsubstituted fluorenyl group, a substituted or unsubstituted dibenzofuranyl group, or a substituted or unsubstituted dibenzothiophenyl group, and a substituted or unsubstituted carbazolyl group.

The fluorenyl groups which can be as A¹, B¹, C¹, A², B², C², and D² may have a substituent at the 9-position, for example, 9,9-dimethylfluorenyl group, and 9,9-diphenylfluorenyl group. Further, the substituents at the 9-position may form a ring with each other, for example, the substituents at the 9-position may form a fluorene skeleton or a xanthene skeleton, with each other.

L^{A1}, L^{B1}, L^{C1}, L^{A2}, L^{B2}, L^{C2} and L^{D2} are preferably each independently a single bond or a substituted or unsubstituted arylene group having 6 to 12 ring carbon atoms.

Specific examples of the compound represented by the formulas (11) and (12) include the following compounds.

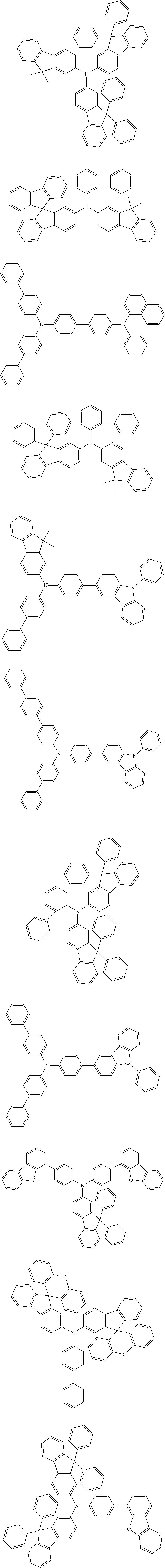

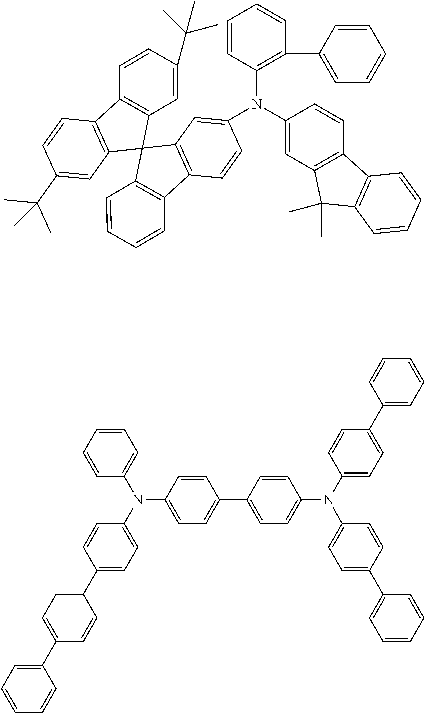 [SMILES: CC(C)(C)c1ccc2c(c1)C1(c3ccccc3-c3ccc(N(c4ccc5c(c4)C(C)(C)c4ccccc4-5)c4ccccc4-c4ccccc4)cc31)c1cc(C(C)(C)C)ccc1-2 ; C1=CC(c2ccc(N(c3ccccc3)c3ccc(-c4ccc(N(c5ccc(-c6ccccc6)cc5)c5ccc(-c6ccccc6)cc5)cc4)cc3)cc2)CC=C1c1ccccc1]

### Dopant Material of the Light Emitting Layer

The light emitting layer is a layer containing a material (dopant material) having a high light emitting properties, and various materials can be used. For example, a fluorescent material or a phosphorescent material can be used as a dopant material. The fluorescent material is a compound which emits light from a singlet excited state, and the phosphorescent material is a compound which emits light from a triplet excited state.

In one embodiment of the organic EL device according to the present invention, the light emitting layer is a single layer.

Further, in one embodiment of the organic EL device according to the present invention, the light emitting layer includes a first light emitting layer and a second light emitting layer.

As a blue fluorescent material which can be used in the light emitting layer, pyrene derivatives, styrylamine derivatives, chrysene derivatives, fluoranthene derivatives, fluorene derivatives, diamine derivatives, triarylamine derivatives, etc. can be used. Specifically, the examples include N,N′-bis[4-(9H-carbazol-9-yl)phenyl]-N,N′-diphaenylstilbene-4,4′-diamine (abbreviation: YGA2S), 4-(9H-carbazol-9-yl)-4′-(10-phenyl-9-anthryl)triphenylamine (abbreviation: YGAPA), and 4-(10-phenyl-9-anthryl)-4′-(9-phenyl-9H-carbazol-3-yl)triphenylamine (abbreviation: PCBAPA).

As a green fluorescent material which can be used in the light emitting layer, aromatic amine derivatives, etc. can be used. Specifically, the examples include N-(9,10-diphenyl-2-anthryl)-N,9-diphenyl-9H-carbazol-3-amine (abbreviation: 2PCAPA), N-[9,10-bis(1,1′-biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(1,1′-biphenyl-2-yl)-2-anthryl]-N,N′,N′-triphenyl-1,4-phenylenediamine (abbreviation: 2DPABPhA), N-[9,10-bis(1,1′-biphenyl-2-yl)]-N-[4-(9H-carbazole)-9-yl)phenyl]-N-phenylanthracen-2-amine (abbreviation: 2YGABPhA), and N,N,9-triphenylanthracen-9-amine (abbreviation: DPhAPhA).

As a red fluorescent material which can be used in the light emitting layer, tetracene derivatives, diamine derivatives, etc. can be used. Specifically, the examples include N,N,N′,N′-tetrakis(4-methylphenyl)tetracene-5,11-diamine (abbreviation: p-mPhTD), and 7,14-diphenyl-N,N,N,N′-tetrakis(4-methylphenyl)acenaphtho[1,2-a]fluoranthene-3,10-diamine (abbreviation: p-mPhAFD).

In one embodiment of the present invention, it is preferable that the light emitting layer contains a fluorescent material (fluorescent dopant material).

As a blue phosphorescent material which can be used in the light emitting layer, metal complexes such as an iridium complex, an osmium complex, a platinum complexes are used. Specifically, the examples include bis[2-(4′,6′-difluorophenyl)pyridinato-N,C2′]iridium(III)tetrakis(1-pyrazolyl)borate (abbreviation: FIr6), bis[2-(4′,6′-difluoro phenyl)pyridinato-N,C2′]iridium(III)picolinate (abbreviation: FIrpic), bis[2-(3′,5′bistrifluoromethylphenyl)pyridinato-N,C2′]iridium(III)picolinate (abbreviation: Ir(CF3ppy)2(pic)), and bis[2-(4′,6′-difluorophenyl)pyridinato-N,C2′]iridium(III)acetylacetonate (abbreviation: FIracac).

As a green phosphorescent material which can be used in the light emitting layer, iridium complexes and the like are used. The examples include tris(2-phenylpyridinato-N,C2′)iridium(III) (abbreviation: Ir(ppy)3), bis(2-phenylpyridinato-N,C2′)iridium(III)acetylacetonate (abbreviation: Ir(ppy)2(acac)), bis(1,2-diphenyl-1H-benzimidazolato)iridium(III)acetylacetonate (abbreviation: Ir(pbi)2(acac)), and bis(benzo[h]quinolinato)iridium(III)acetylacetonate (abbreviation: Ir(bzq)2(acac)).

As a red phosphorescent material which can be used in the light emitting layer, metal complexes such as an iridium complex, a platinum complex, a terbium complex, and an europium complex are used. Specifically; the examples include organic metal complexes such as bis[2-(2′-benzo[4,5-a]thienyl)pyridinato-N,C3′]iridium(III)acetylacetonate (abbreviation: Ir(btp)2(acac)), bis(1-phenylisoquinolinato-N,C2′)iridium(III)acetylacetonate (abbreviation: Ir(piq)2(acac)), (acetylacetonato)bis[2,3-bis(4-fluorophenyl)quinoxalinato]iridium(III) (abbreviation: Ir(Fdpq)2(acac)), and 2,3,7,8,12,13,17,18-octaethyl-21H,23H-porphyrin platinum(II) (abbreviation: PtOEP).

Further, rare earth metal complexes such as tris(acetylacetonato)(monophenanthroline)terbium(III) (abbreviation: Tb(acac)3(Phen)), tris(1,3-diphenyl-1,3-propanedionato)(monophenanthroline)europium(III) (abbreviation: Eu(DBM)3(Phen)), and tris[1-(2-thenoyl)-3,3,3-trifluoroacetonato](monophenanthroline)europium(III) (abbreviation: Eu(TTA)3(Phen)) can be used as the phosphorescent material since they emit light from rare earth metal ions (electronic transition between different multiplicities).

### Host Material of the Light Emitting Layer

The light emitting layer may have a configuration in which the above-described dopant material is dispersed in another material (host material). It is preferable to use a material which has a higher lowest unoccupied orbital level (LUMO level) and a lower highest occupied orbital level (HOMO level) than the dopant material.

As the host material, for example,
(1) metal complexes such as an aluminum complex, a beryllium complex, or a zinc complex,
(2) heterocyclic compounds such as an oxadiazole derivative, a benzimidazole derivative, or a phenanthroline derivative,
(3) condensed aromatic compounds such as a carbazole derivative, an anthracene derivative, a phenanthrene derivative, a pyrene derivative, or a chrysene derivative, and
(4) aromatic amine compounds such as a triarylamine derivative, or a condensed polycyclic aromatic amine derivative, are used.

For example, metal complexes such as tris(8-quinolinolato)aluminum(III) (abbreviation: Alq), tris(4-methyl-8-quinolinolato)aluminum(III) (abbreviation: Almq3), bis(10)-hydroxybenzo[h]quinolinato)beryllium(II) (abbreviation: BeBq2), bis(2-methyl-8-quinolinolato)(4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), and bis[2-(2-benzothiazolyl)phenolato]zinc(II) (abbreviation: ZnBTZ);
heterocyclic compounds such as 2-(4-biphenylyl)-5-(4-tert-butylphenyl)-1,3,4-oxadiazole (abbreviation: PBD), 1,3-bis[5-(p-tert-butylphenyl)-1,3,4-oxadiazol-2-yl]benzene (abbreviation: OXD-7), 3-(4-biphenylyl)-4-phenyl-5-(4-tert-butylphenyl)-1,2,4-triazole (abbreviation: TAZ), 2,2′,2″-(1,3,5-benzentriyl)tris(1-phenyl-1H-benzimidazole) (abbreviation: TPBI), bathophenanthroline (abbreviation: BPhen), and bathocuproine (abbreviation: BCP):

condensed aromatic compounds such as 9-[4-(10-phenyl-9-anthryl)phenyl]-9H-carbazole (abbreviation: CzPA), 3,6-diphenyl-9-[4-(10-phenyl-9-anthryl)phenyl]-9H-carbazole (abbreviation: DPCzPA), 9,10-bis(3,5-diphenylphenyl)anthracene (abbreviation: DPPA), 9,10-di(2-naphthyl)anthracene (abbreviation: DNA), 2-tert-butyl-9,10-di(2-naphthyl)anthracene (abbreviation: t-BuDNA), 9,9′-bianthryl (abbreviation: BANT), 9,9′-(stilbene-3,3′-diyl)diphenanthrene (abbreviation: DPNS), 9,9′-(stilbene-4,4′-diyl)diphenanthrene (abbreviation: DPNS2), 3,3′,3″-(benzene-1,3,5-triyl)tripyrene (abbreviation: TPB3), 9,10-diphenylanthracene (abbreviation: DPAnth), and 6,12-dimethoxy-5,11-diphenylchrysene; and aromatic amine compounds such as N,N-diphenyl-9-[4-(10-phenyl-9-anthryl)phenyl]-9H-carbazol-3-amine (abbreviation: CzAIPA), 4-(10)-phenyl-9)-anthryl)triphenylamine (abbreviation: DPhPA), N,9-diphenyl-N-[4-(10)-phenyl-9)-anthryl)phenyl]-9H-carbazol-3-amine (abbreviation: PCAPA), N,9-diphenyl-N-{4-[4-(10-phenyl-9-anthryl)phenyl]phenyl}-9H-carbazol-3-amine (abbreviation: PCAPBA), N-(9,10-diphenyl-2-anthryl)-N,9-diphenyl-9H-carbazol-3-amine (abbreviation: 2PCAPA), 4,4′-bis[N-(1-naphthyl)-N-phenylamino]biphenyl (abbreviation: NPB or α-NPD), N,N′-bis(3-methylphenyl)-N,N′-diphenyl-[1,1′-biphenyl]-4,4′-diamine (abbreviation: TPD), 4,4′-bis[N-(9,9-dimethylfluoren-2-yl)-N-phenylamino]biphenyl (abbreviation: DFLDPBi), and 4,4′-bis[N-(spiro-9,9′-bifluoren-2-yl)-N-phenylamino]biphenyl (abbreviation: BSPB): can be used. Multiple types of host materials may be used.

In particular, in the case of a blue fluorescent element, it is preferable to use the following anthracene compound as the host material.

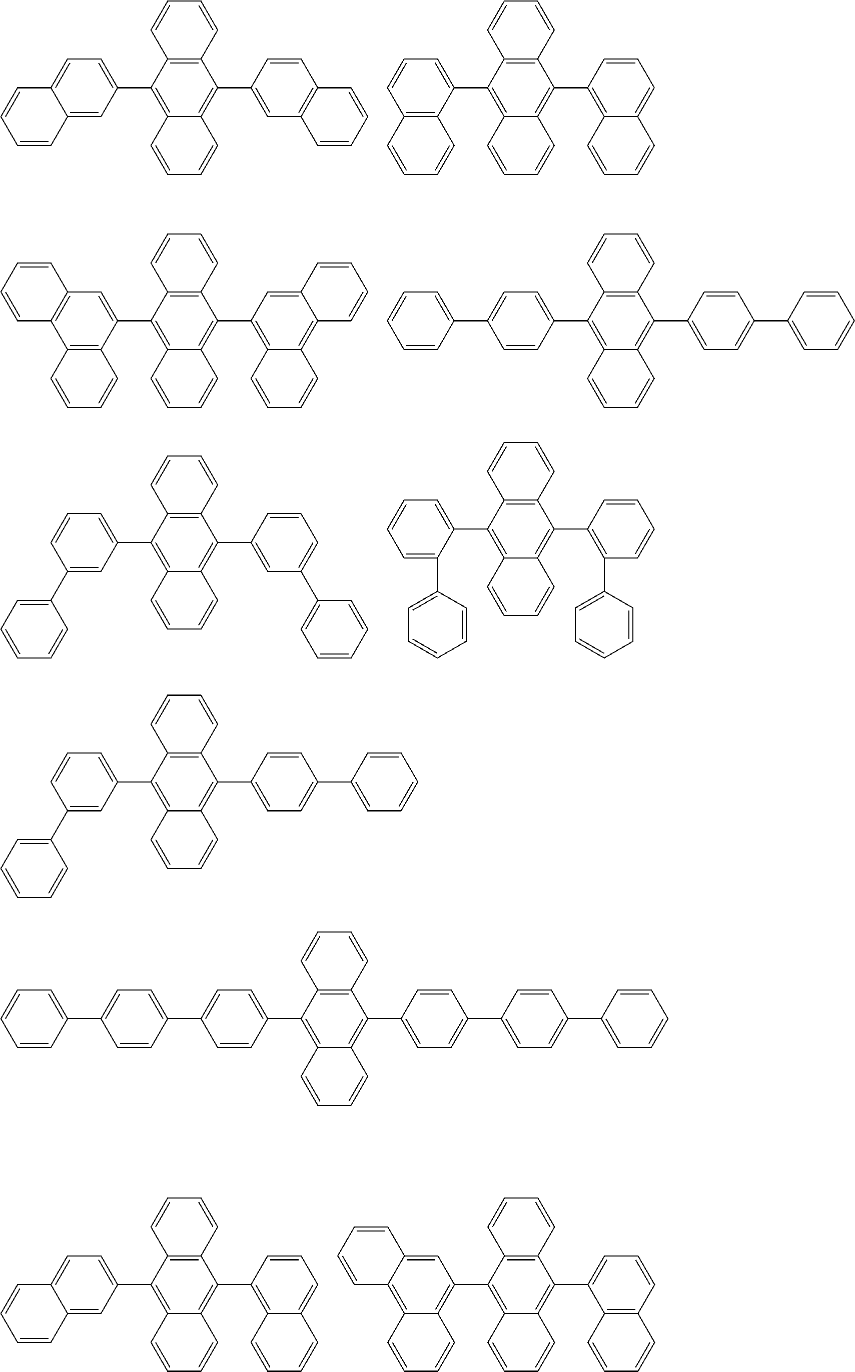

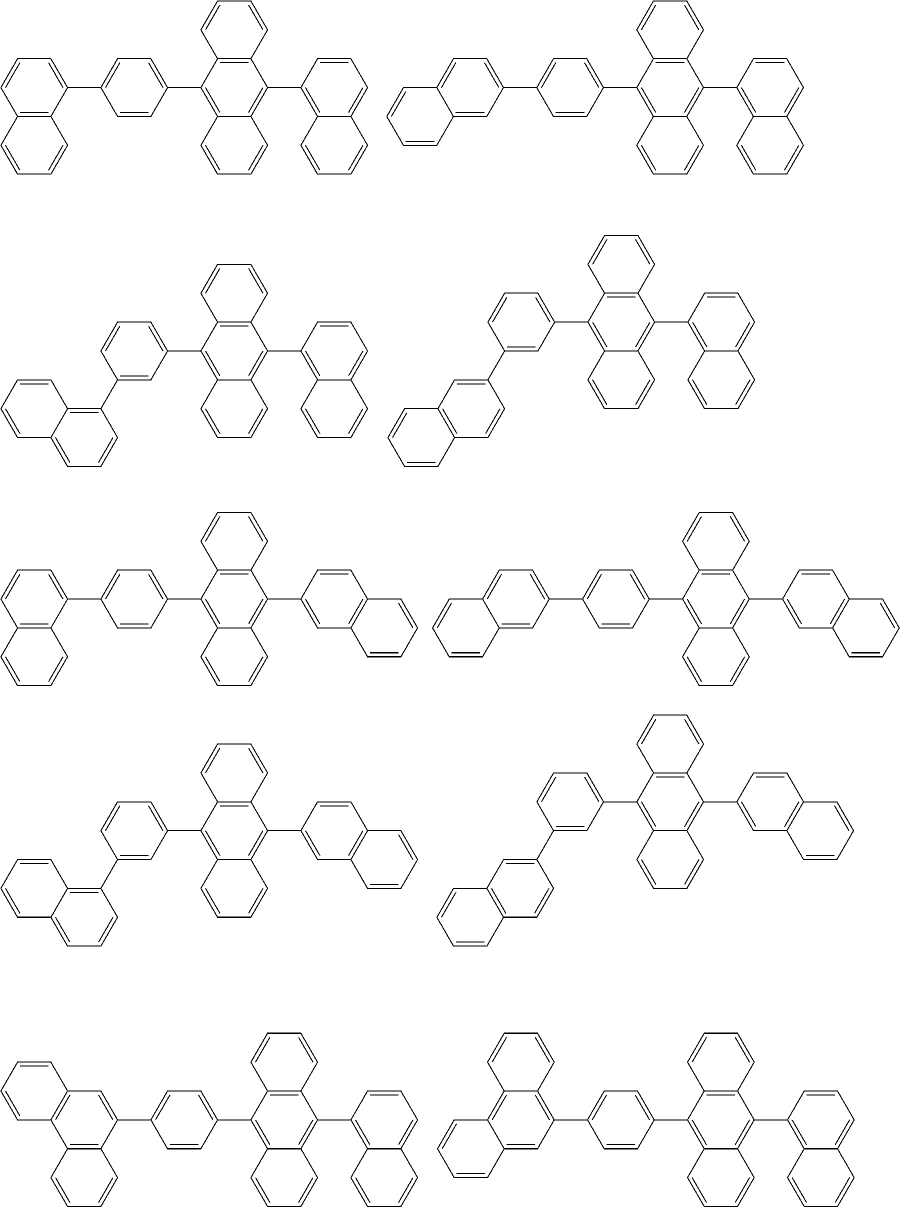

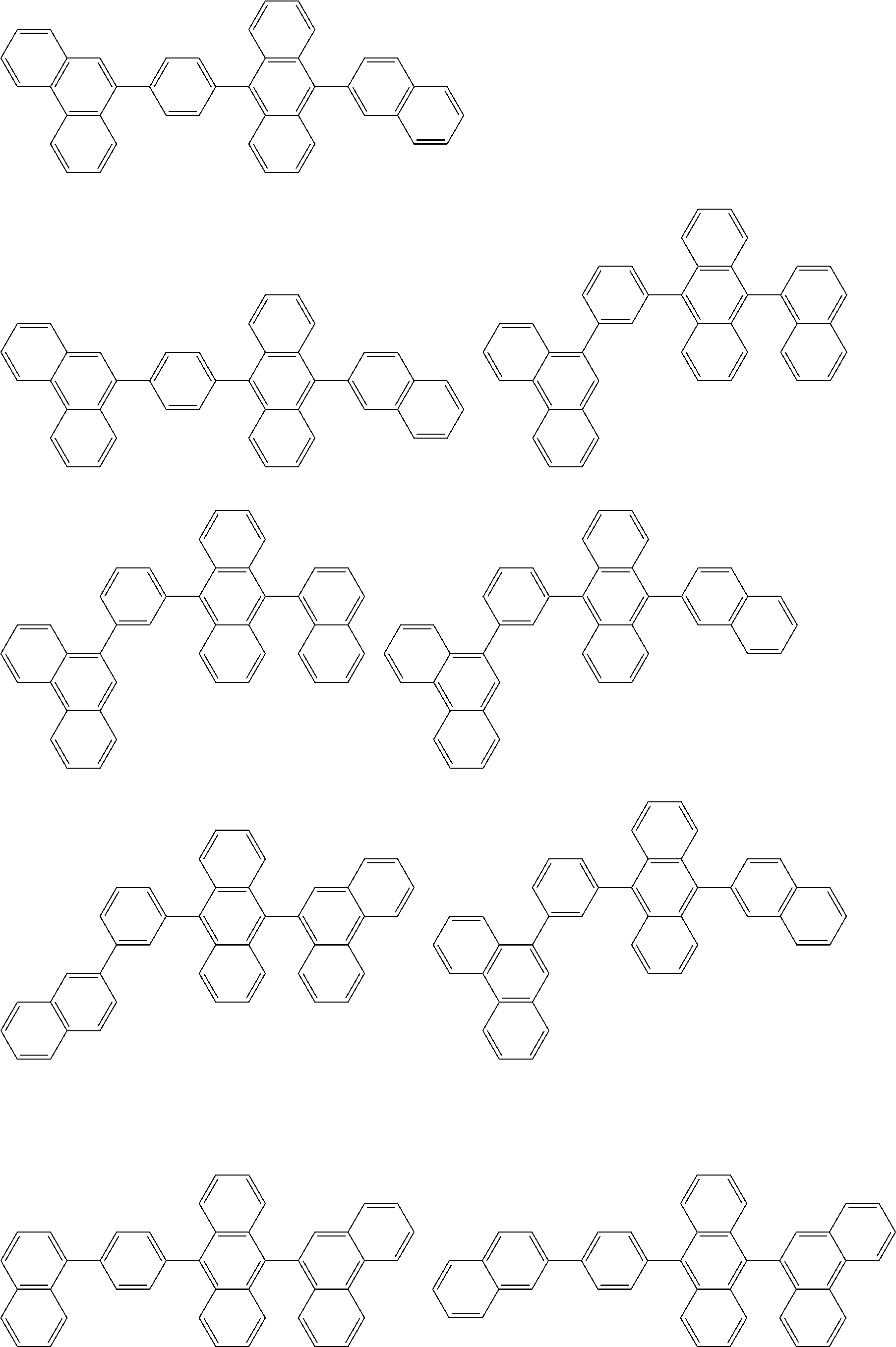

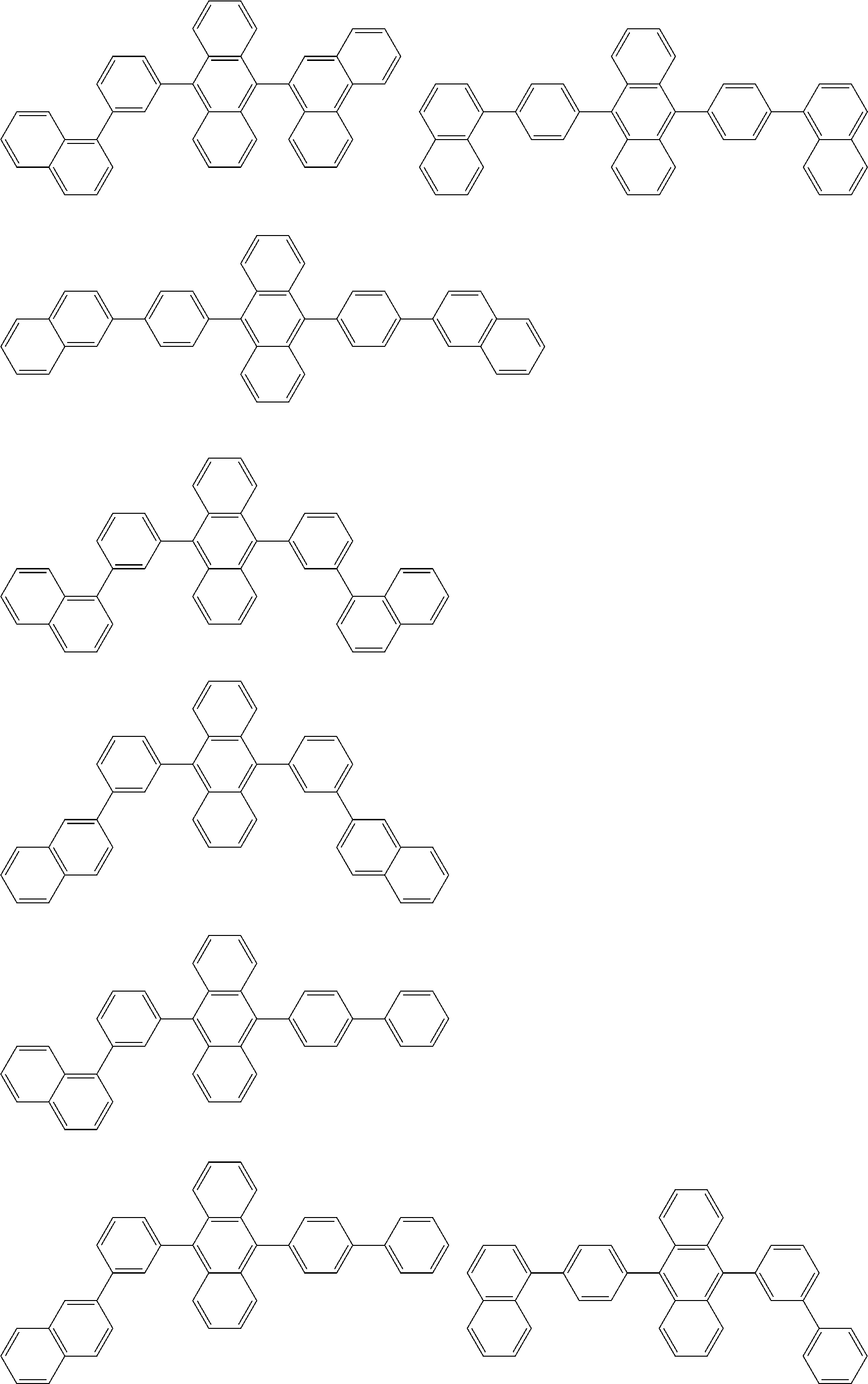

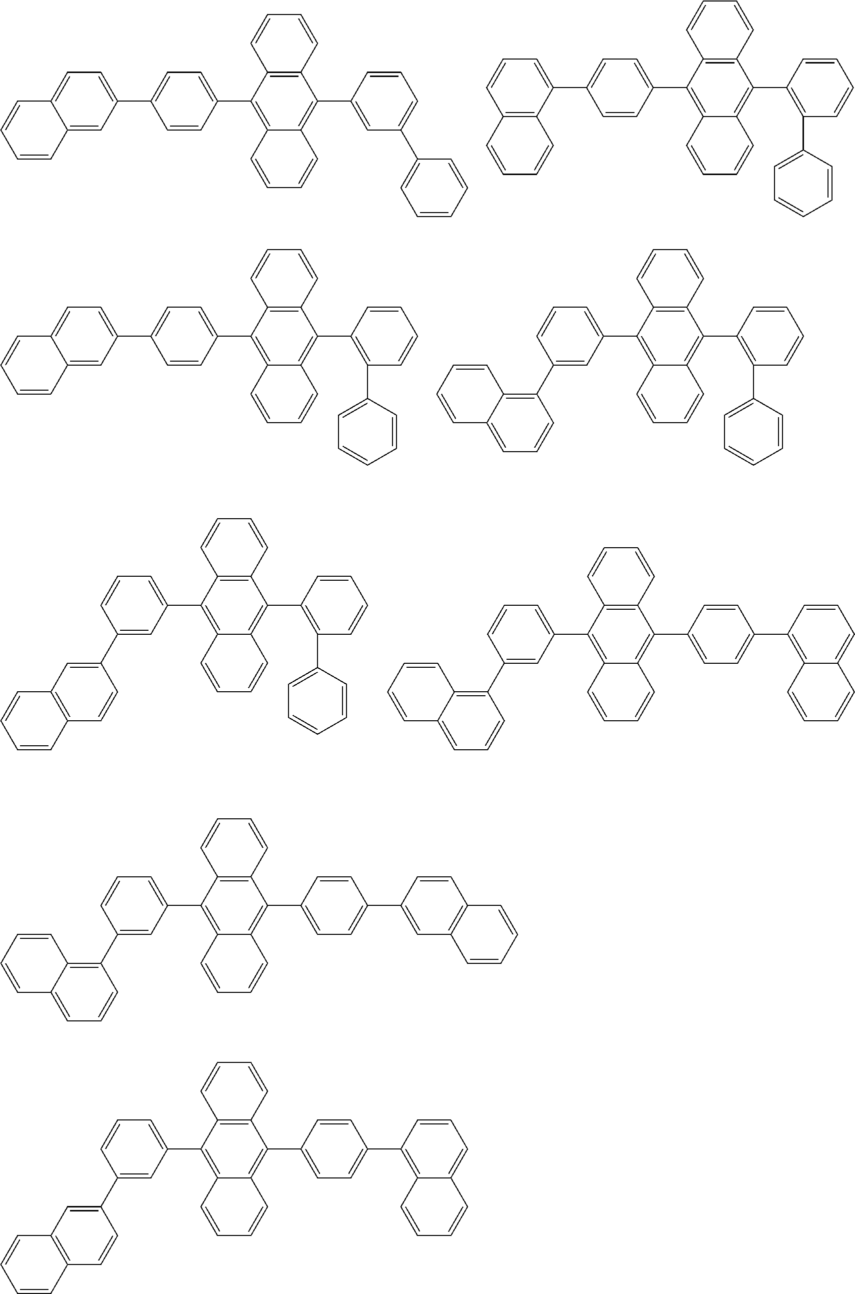

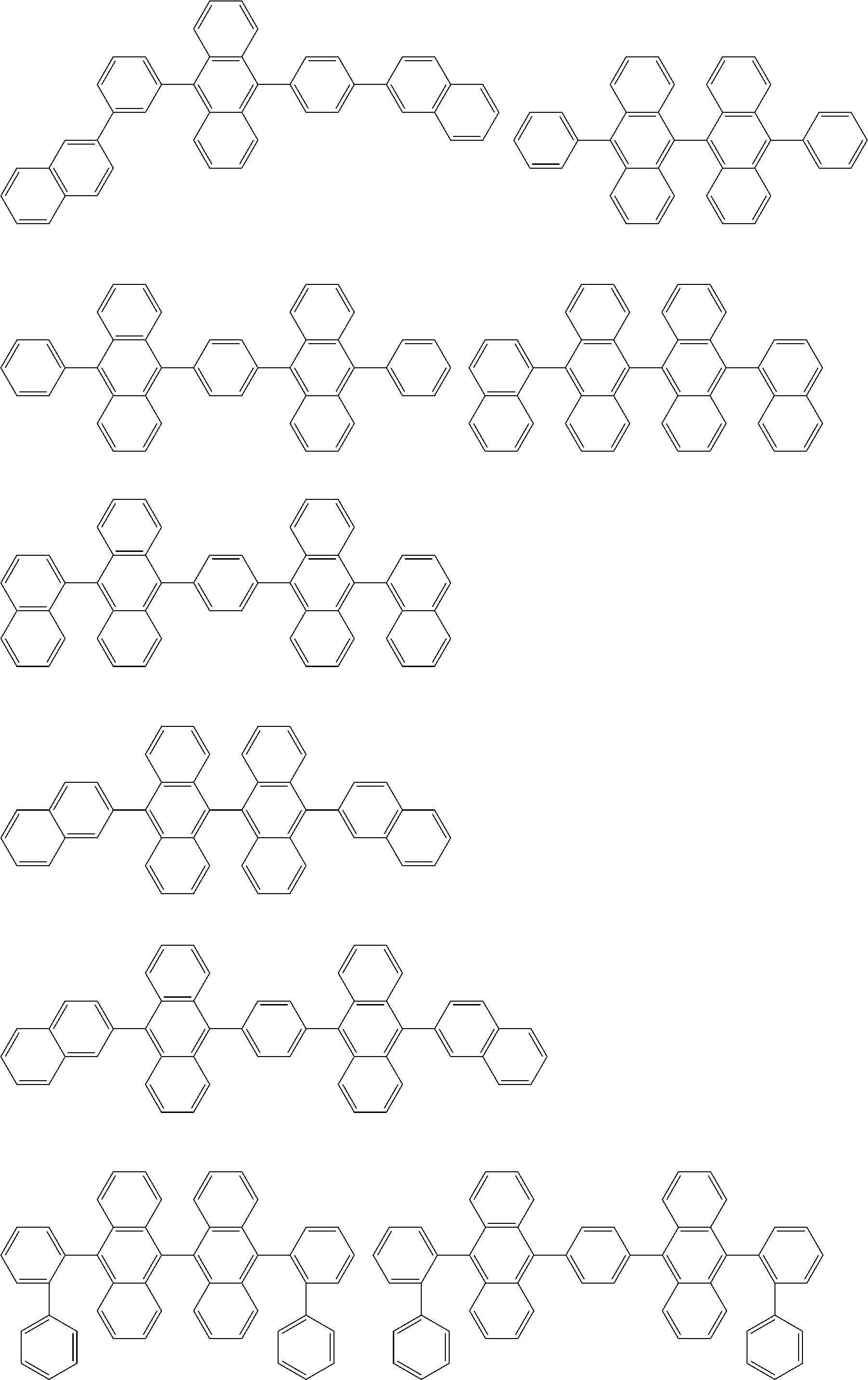

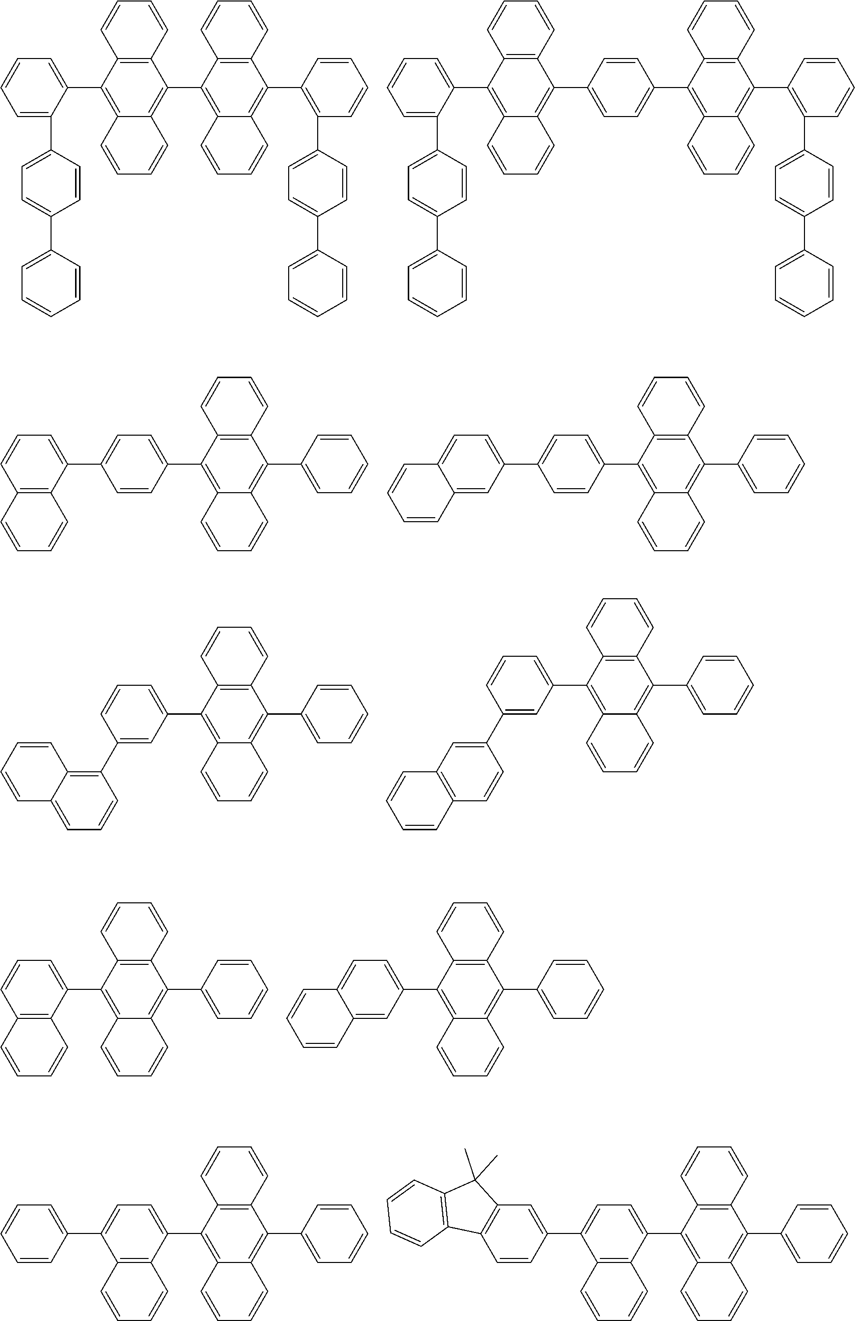

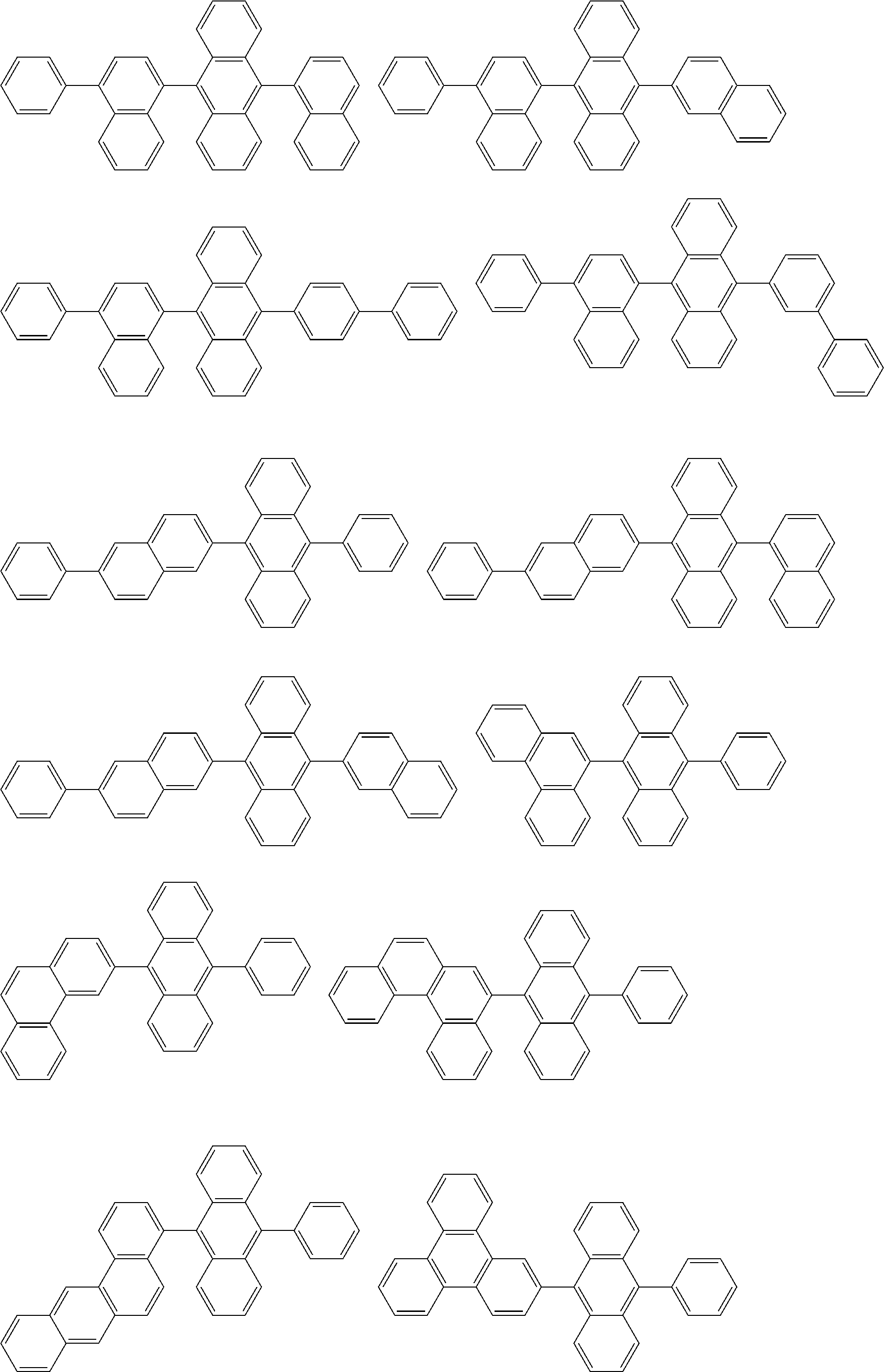

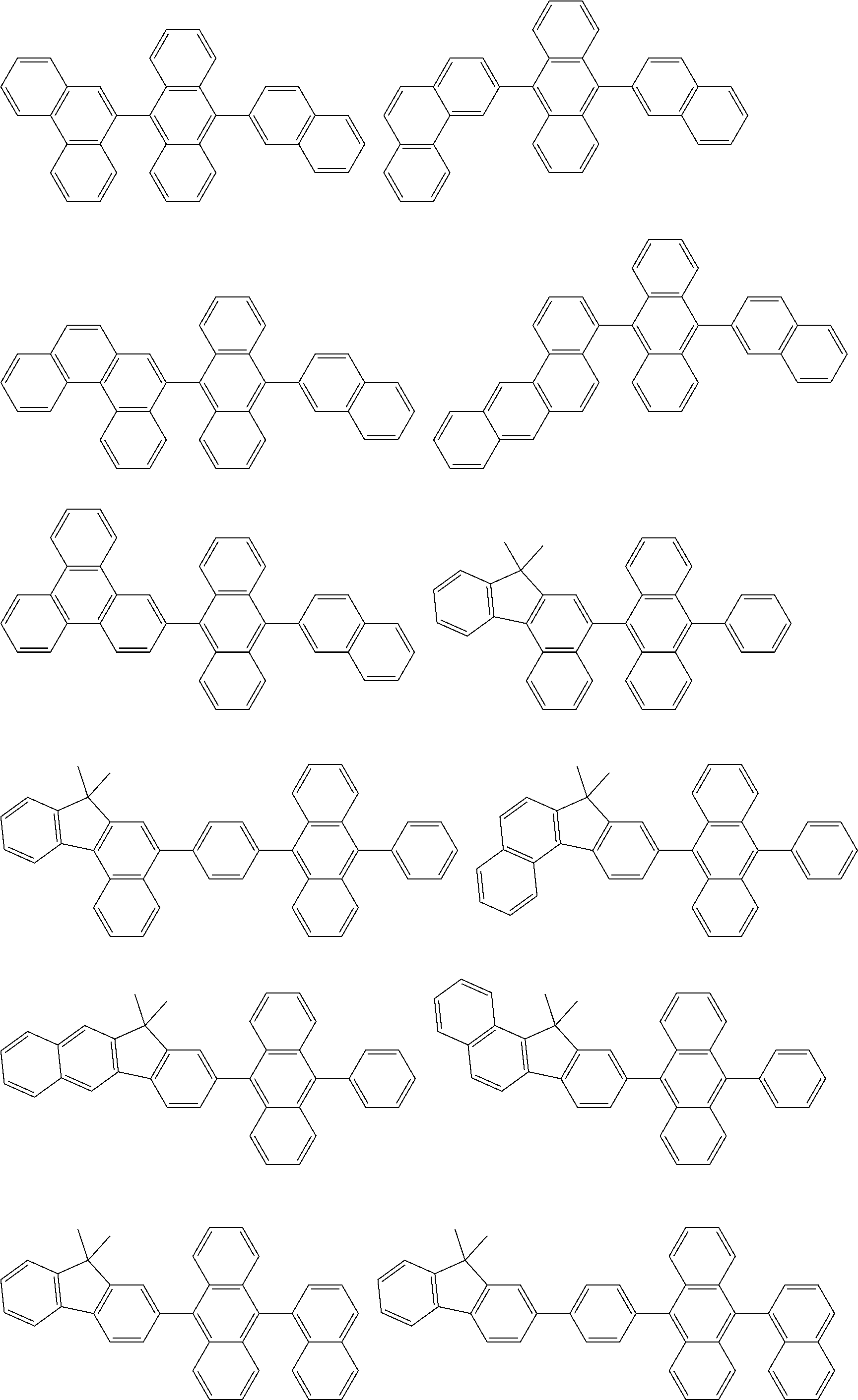

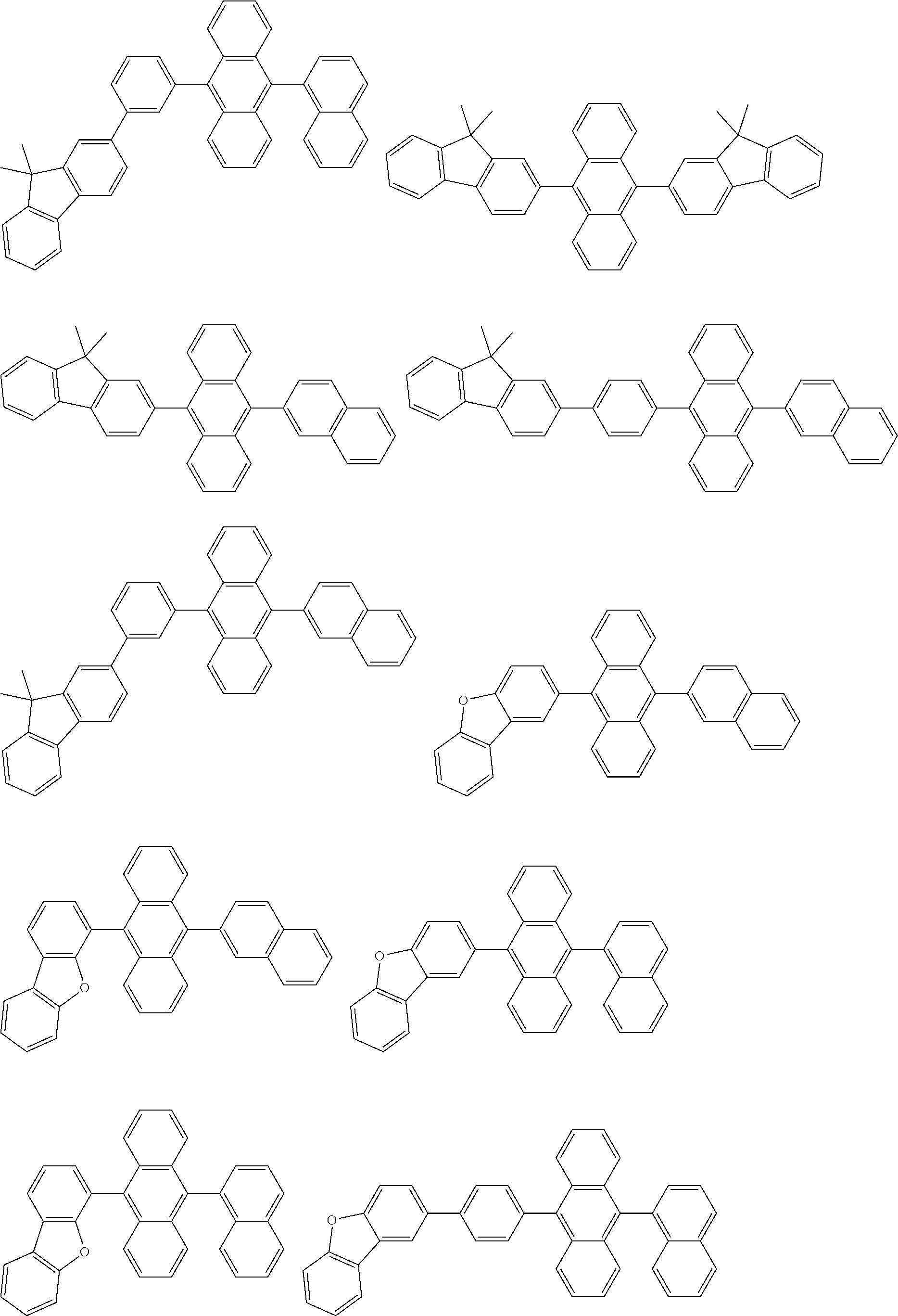

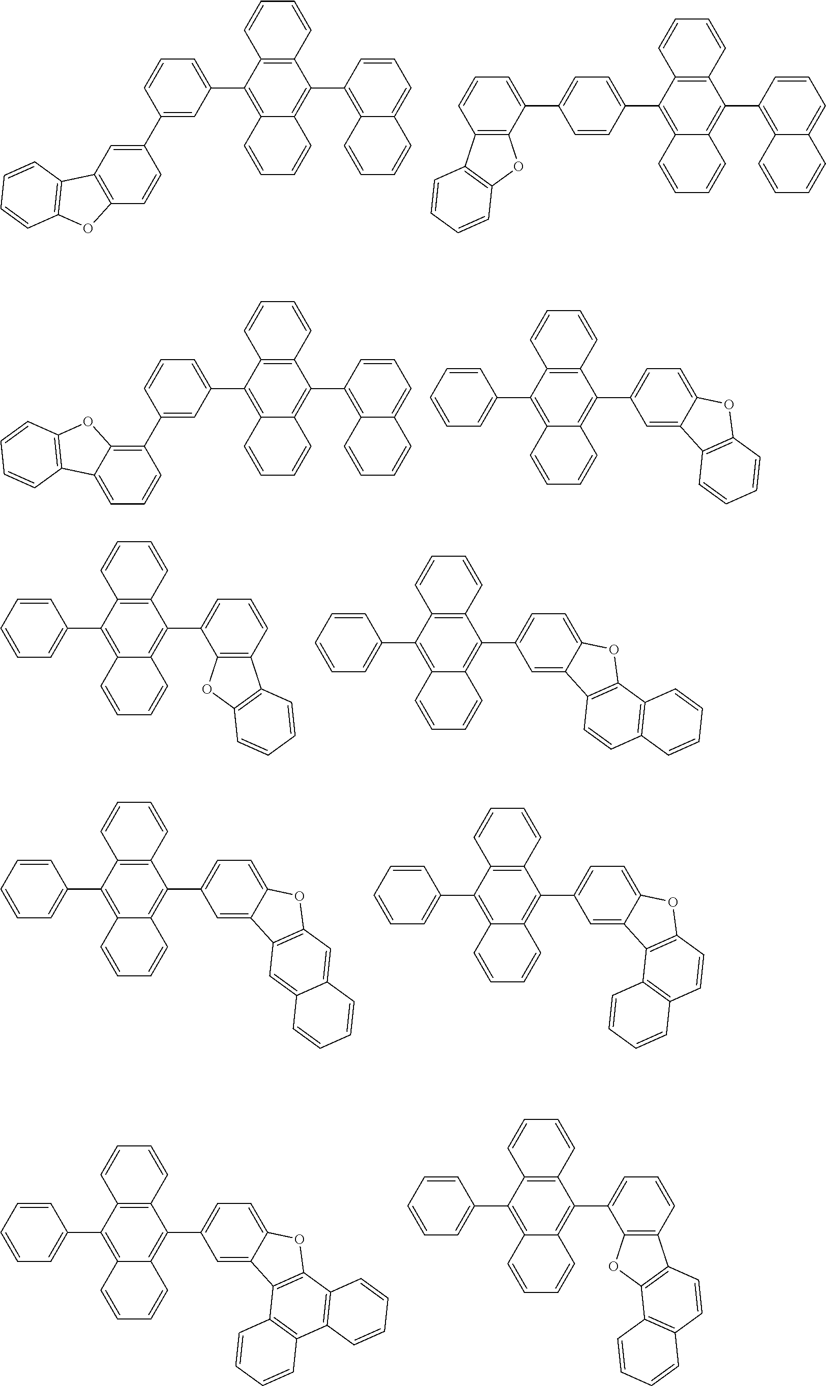

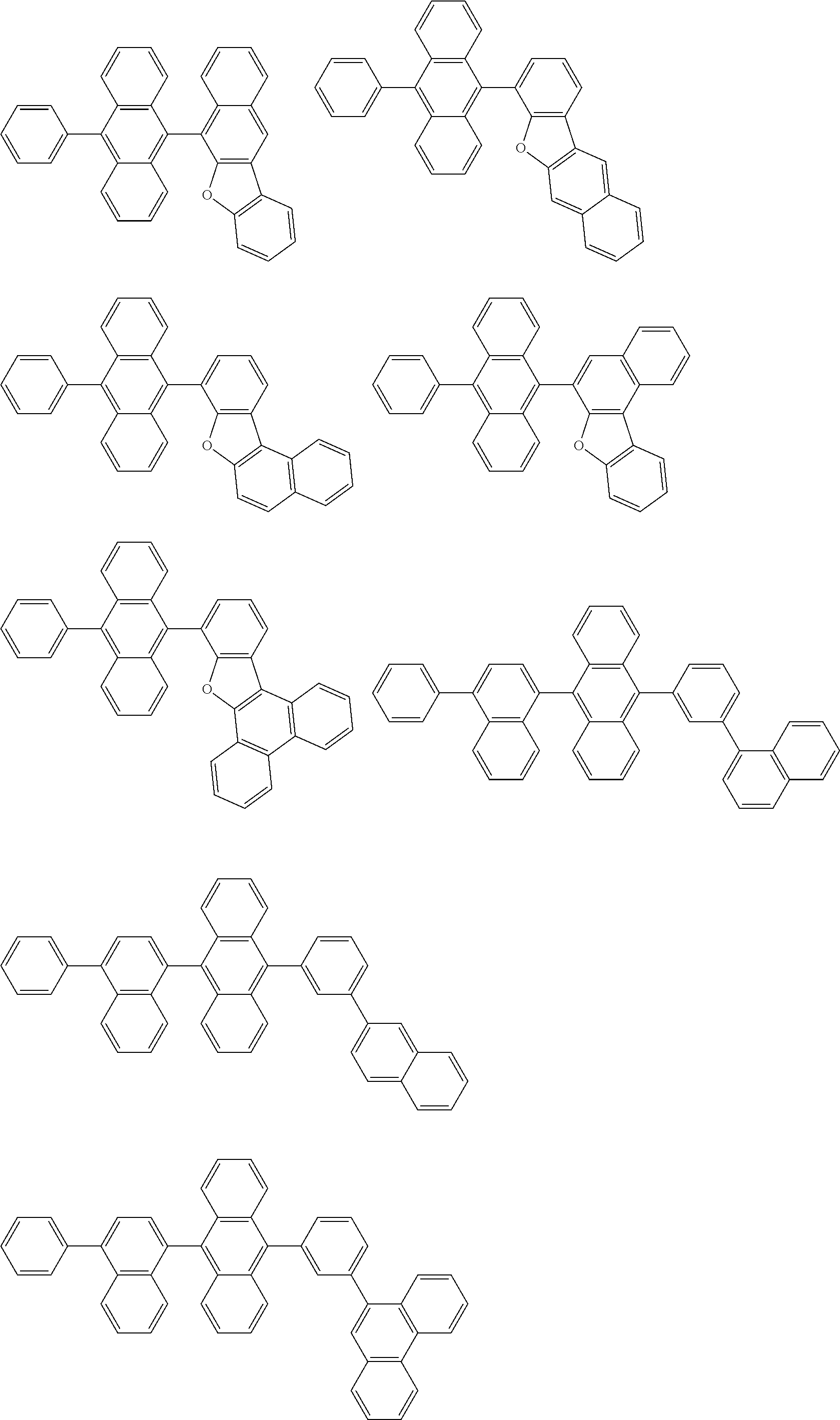

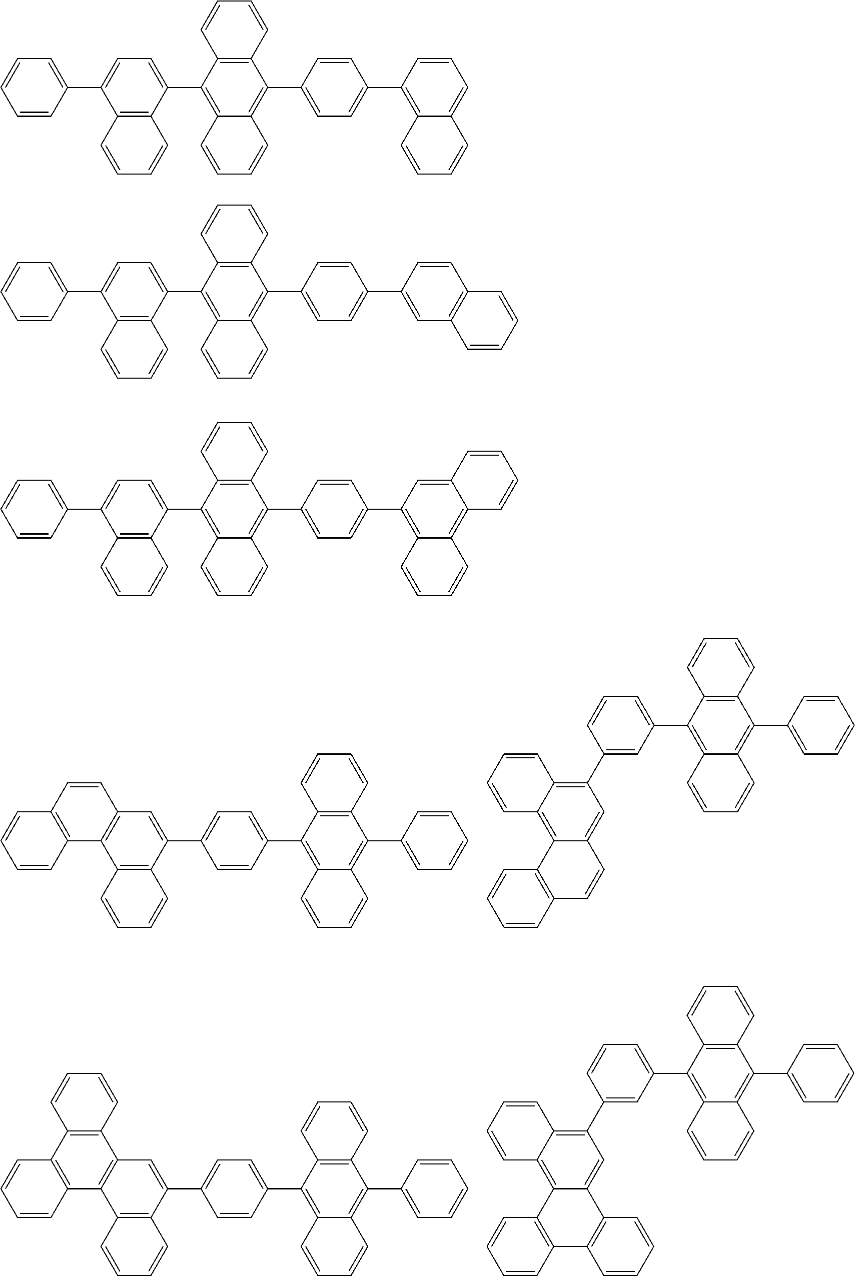

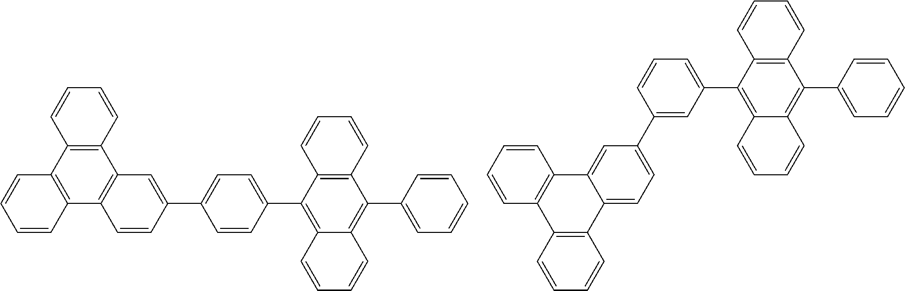

In one embodiment of the organic EL device according to the present invention, when the light emitting layer contains a first light emitting layer and a second light emitting layer, at least one of the components constituting the first light emitting layer is different from the components constituting the second light emitting layer. For example, the examples include an embodiment in which the dopant material contained in the first light emitting layer is different from the dopant material contained in the second light emitting layer, and an embodiment in which the host material contained in the first light emitting layer is different from the host material contained in the second light emitting layer.

In the organic EL device of the present invention, the light emitting layer may contain a light emitting compound (hereinafter sometimes simply referred to as a “fluorescent compound”) which emits fluorescent light with a main peak wavelength of 500 nm or less.

The method for measuring the main peak wavelength of a compound is as follows. A 5 μmol/L toluene solution of the compound to be measured is prepared and placed in a quartz cell, and the emission spectrum of this sample (vertical axis: emission intensity, horizontal axis: wavelength) is measured at room temperature (300K). The emission spectrum can be measured using a spectrofluorometer (device name: F-7000) manufactured by Hitachi High-Tech Science Co., Ltd. Note that the emission spectrum measuring device is not limited to the device used here.

In the emission spectrum, the peak wavelength of the emission spectrum at which the emission intensity is maximum is defined as the main peak wavelength. Note that in this specification, the main peak wavelength may be referred to as fluorescence main peak wavelength (FL-peak).

The fluorescent compound may be the dopant material or the host material.

When the light emitting layer is a single layer, only one of the dopant material and the host material may be the fluorescent compound, or both may be the fluorescent compound.

Further, when the light emitting layer contains a first light emitting layer (on the anode side) and a second light emitting layer (on the cathode side), only one of the first light emitting layer and the second light emitting layer contains the fluorescent compound, or both of the light emitting layers may contain the fluorescent compound. When the first light emitting layer contains the fluorescent compound, only one of the dopant material and the host material contained in the first light emitting layer may be the fluorescent compound, or both may be the fluorescent compound. Further, when the second light emitting layer contains the fluorescent compound, only one of the dopant material and the host material contained in the second light emitting layer may be the fluorescent compound, or both may be the fluorescent compound.

### Electron Transporting Layer

The electron transporting layer is a layer containing a material having high electron transport properties (electron transporting material), and is formed between the light emitting layer and the cathode, or, between the electron injecting layer and the light emitting layer when present.

The electron transporting layer may have a single-layer structure or a multilayer structure including two or more layers. For example, the electron transporting layer may have a two-layer structure including a first electron transporting layer (on the anode side) and a second electron transporting layer (on the cathode side). In one embodiment of the present invention, the electron transporting layer having the single-layer structure is preferably adjacent to the light emitting layer, and the electron transporting layer closest to the anode in the multilayer structure, which is, for example, the first electron transporting layer in the two-layer structure, is preferably adjacent to the light emitting layer. In another embodiment of the present invention, a hole blocking layer, which will be described later, may be interposed between the electron transporting layer and the light emitting layer in the single-layer structure, or between the electron transporting layer and the light emitting layer closest to the light emitting layer in the multilayer structure.

In the electron transporting layer,
(1) metal complexes such as an aluminum complex, a beryllium complex, and a zinc complex,
(2) heteroaromatic compounds such as an imidazole derivative, a benzimidazole derivative, an azine derivative, a carbazole derivative, and a phenanthroline derivative, and
(3) high molecular weight compounds, can be used.

Examples of the metal complex include tris(8-quinolinolato)aluminum(III) (abbreviation: Alq), tris(4-methyl-8-quinolinolato)aluminum(abbreviation: Almq3), bis(10-hydroxybenzo[h]quinolinato) beryllium (abbreviation: BeBq2), bis(2-methyl-8-quinolinolato)(4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), and bis[2-(2-benzothiazolyl)phenolato]zinc(II) (abbreviation: ZnBTZ).

Examples of the heteroaromatic compound include 2-(4-biphenylyl)-5-(4-tert-butylphenyl)-1,3,4-oxadiazole (abbreviation: PBD), 1,3-bis[5-(ptert-butylphenyl)-1,3,4-oxadiazol-2-yl]benzene (abbreviation: OXD-7), 3-(4-tert-butylphenyl)-4-phenyl-5-(4-biphenylyl)-1,2,4-triazole (abbreviation: TAZ), 3-(4-tert-butylphenyl)-4-(4-ethylphenyl)-5-(4-biphenylyl)-1,2,4-triazole (abbreviation: p-EtTAZ), bathophenanthroline (abbreviation: BPhen), bathocuproine (abbreviation: BCP), and 4,4′-bis(5-methylbenzoxazol-2-yl) stilbene (abbreviation: BzOs).

Examples of the high molecular weight compound include poly[(9,9-dihexylfluorene-2,7-diyl)-co-(pyridine-3,5-diyl)] (abbreviation: PF-Py), poly[(9,9-dioctylfluorene-2,7-diyl)-co-(2,2′-bipyridine-6,6′-diyl)] (abbreviation: PF-BPy).

The material has an electron mobility of 10⁻⁶ cm²/Vs or more. Note that materials other than those mentioned above may be used for the electron transporting layer as long as they have higher electron transport properties than hole transport properties.

### Electron Injecting Layer

The electron injecting layer is a layer containing a material having high electron injection properties. In the electron injecting layer, alkali metals such as lithium (Li) and cesium (Cs), alkaline earth metals such as magnesium (Mg), calcium (Ca), and strontium (Sr), rare earth metals such as a europium (Eu), and ytterbium (Yb), and compounds containing these metals, can be used. Examples of such compounds include alkali metal oxides, alkali metal halides, alkali metal-containing organic complexes, alkaline earth metal oxides, alkaline earth metal halides, alkaline earth metal-containing organic complexes, rare earth metal oxides, rare earth metal halides, and rare earth metal-containing organic complexes. Further, a plurality of these compounds can also be used in combination.

In addition, a material having an electron transport property in which alkali metals, alkaline earth metals, or compounds thereof are contained, specifically, the Alq in which magnesium (Mg) is contained, may be used. Note that in this case, the electron injection from the cathode can be effectively performed.

Alternatively, a composite material formed by mixing an organic compound and an electron donor (donor) in the electron injecting layer. Such a composite material has excellent electron injection properties and electron transport properties since the organic compound receives electrons from an electron donor. In this case, the organic compound is preferably a material which is excellent in transporting received electrons, and specifically, for example, the above-mentioned materials constituting the electron transporting layer (metal complexes, heteroaromatic compounds, etc.) can be used. The electron donor may be any material as long as it exhibits electron donating properties to organic compounds. Specifically, alkali metals, alkaline earth metals, and rare earth metals are preferred, and examples thereof include lithium, cesium, magnesium, calcium, erbium, and ytterbium. Moreover, alkali metal oxides and alkaline earth metal oxides are preferred, and examples thereof include lithium oxide, calcium oxide, and barium oxide. In addition, Lewis bases such as magnesium oxide can also be used. Moreover, organic compounds such as tetrathiafulvalene (abbreviation: TTF) can also be used.

### Cathode

For the cathode, it is preferable to use a metal, an alloy, an electrically conductive compound, a mixture thereof, or the like having a small work function (specifically, 3.8 eV or less). Specific examples of the cathode material include elements belonging to Group 1 or Group 2 of the Periodic Table of Elements, that is, alkali metals such as lithium (Li) and cesium (Cs), and alkaline earth metals such as magnesium (Mg), calcium (Ca), and strontium (Sr), alloys containing these (for example, MgAg, AlLi), rare earth metals such as europium (Eu), ytterbium (Yb), and alloys containing these.

In addition, when forming a cathode using an alkali metal, an alkaline earth metal, or an alloy containing these, a vacuum deposition method or a sputtering method can be used. Furthermore, when silver paste or the like is used, a coating method, an inkjet method, or the like can be used.

By providing an electron injecting layer, the cathode can be formed using various conductive materials such as Al, Ag, ITO, graphene, and indium oxide-tin oxide containing silicon or silicon oxide, no matter how large or small the work function is. These conductive materials can be film-formed by using a sputtering method, an inkjet method, a spin coating method, or the like.

### Insulating Layer

Since organic EL devices apply an electric field to an ultra-thin film, pixel defects are likely to occur due to leakage or short circuits. In order to prevent this, an insulating layer made of an insulating thin film layer may be inserted between a pair of electrodes.

Examples of materials used for the insulating layer include aluminum oxide, lithium fluoride, lithium oxide, cesium fluoride, cesium oxide, magnesium oxide, magnesium fluoride, calcium oxide, calcium fluoride, aluminum nitride, titanium oxide, silicon oxide, germanium oxide, silicon nitride, boron nitride, molybdenum oxide, ruthenium oxide, and vanadium oxide. Note that a mixture or a laminate of these may also be used.

### Space Layer

The space layer is a layer provided between, for example, when laminating a fluorescent layer and a phosphorescent layer, the fluorescent light emitting layer and the phosphorescent light emitting layer, in order to prevent excitons generated in the phosphorescent layer from diffusing into the fluorescent layer or to adjust the carrier balance, for example. Moreover, the space layer can also be provided between plural phosphorescent light emitting layers.

Since the space layer is provided between the light emitting layers, the material for the space layer preferably has both electron transporting properties and hole transporting properties. Further, in order to prevent triplet energy from diffusing in adjacent phosphorescent light emitting layers, the triplet energy of the material is preferably 2.6 eV or more. Examples of the material used for the space layer include the same materials as those used for the hole transporting layer described above.

### Blocking Layer

Blocking layers such as an electron blocking layer, a hole blocking layer, and an exciton blocking layer may be provided adjacent to the light emitting layer. The electron blocking layer is a layer which prevents electrons from leaking from the light emitting layer to the hole transporting layer, and the hole blocking layer is a layer which prevents holes from leaking from the light emitting layer to the electron transporting layer. The exciton blocking layer has the function of preventing excitons generated in the light emitting layer from diffusing into surrounding layers and confining the excitons within the light emitting layer.

Each layer of the organic EL device can be formed by a conventionally known vapor deposition method, coating method, or the like. For example, vapor deposition methods such as vacuum deposition method and molecular beam evaporation method (MBE method), or known coating methods such as dipping method, spin coating method, casting method, bar coating method, and roll coating method which use a solution of a compound forming a layer, can be used for forming.

The film thickness of each layer is not particularly limited, however in general, when the film thickness is too thin, defects such as pinholes are likely to occur, and on the other hand, when the film thickness is too thick, a high drive voltage will be required and efficiency will deteriorate, therefore it is usually 5 nm to 10 μm, more preferably 10 nm to 0.2 μm.

In the organic EL device having the hole transporting layer having the two-layer structure or the three-layer structure of the present invention, the total of the thickness of the first hole transporting layer and the thickness of the second hole transporting layer is preferably 30 nm or more and 150 nm or less, more preferably 40 nm or more and 130 nm or less.

Further, in one embodiment of the present invention, the thickness of the second hole transporting layer having the two-layer structure or the three-layer structure is preferably 5 nm or more, more preferably 20 nm or more, further preferably 25 nm or more, particularly preferably 35 nm or more, and preferably 100 nm or less.

Further, in one embodiment of the present invention, the thickness of the hole transporting layer which is adjacent to the light emitting layer is preferably 5 nm or more, more preferably 20 nm or more, further preferably 25 nm or more, particularly preferably 30 nm or more, and preferably 100 nm or less.

Further, in the organic EL device having the hole transporting layer having the two-layer structure or the three-layer structure according to the present invention, the ratio of the film thickness D2 of the second hole transporting layer to the film thickness D1 of the first hole transporting layer is preferably 0.3<D2/D1<4.0, more preferably 0.5<D2/D1<3.5, further preferably 0.75<D2/D1<3.0.

Preferred embodiments of the organic EL device of the present invention include, for example,
(1) organic EL device having a hole transporting layer of two-layer configuration:
first embodiment in which the second hole transporting layer contains the inventive compound and the first hole transporting layer does not contain the inventive compound;
second embodiment in which both the first hole transporting layer and the second hole transporting layer contain the inventive compound; and
third embodiment in which the first hole transporting layer contains the inventive compound and the second hole transporting layer does not contain the inventive compound,
(2) organic EL device having a hole transporting layer of three-layer configuration:
fourth embodiment in which the first hole transporting layer contains the inventive compound and the second and the third hole transporting layers do not contain the inventive compound;
fifth embodiment in which the second hole transporting layer contains the inventive compound and the first and the third hole transporting layers do not contain the inventive compound;
sixth embodiment in which the third hole transporting layer contains the inventive compound and the first and the second hole transporting layers do not contain the inventive compound;
seventh embodiment in which the first and the second hole transporting layers contain the inventive compound and the third hole transporting layer does not contain the inventive compound;
eighth embodiment in which the first and the third hole transporting layers contain the inventive compound and the second hole transporting layer does not contain the inventive compound;
ninth embodiment in which the second and the third hole transporting layers contain the inventive compound and the first hole transporting layer does not contain the inventive compound; and
tenth embodiment in which all of the first to third hole transporting layers contain the inventive compound.

### Electronic Device

The organic EL device can be used in display parts such as organic EL panel modules, display devices such as televisions, mobile phones, and personal computers, and electronic devices such as lighting and light emitting devices of vehicle lamps.

### EXAMPLE

Hereinafter, the present invention will be described in more detail by the following examples, however the present invention is not limited to the following examples.

Inventive compounds used for producing the organic EL devices of Examples 1 to 7

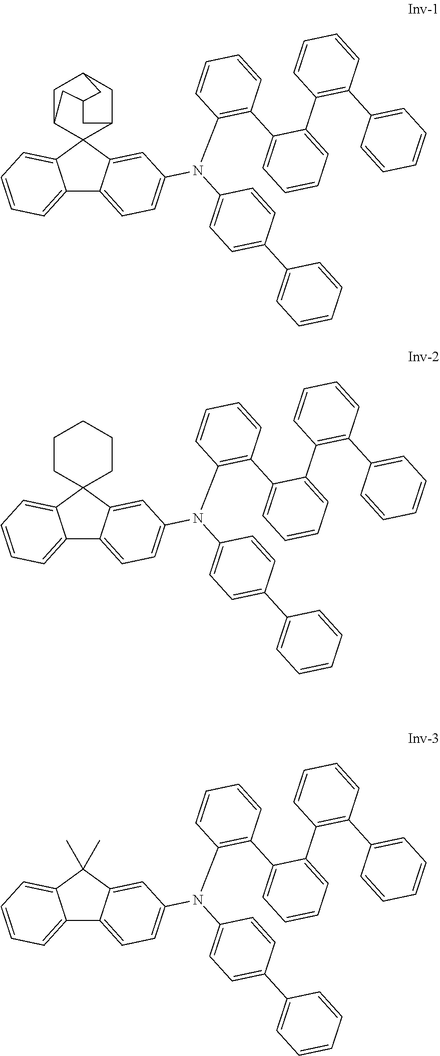 [SMILES: c1ccc(-c2ccc(N(c3ccc4c(c3)C3(c5ccccc5-4)C4CC5CC(C4)CC3C5)c3ccccc3-c3ccccc3-c3ccccc3-c3ccccc3)cc2)cc1 ; c1ccc(-c2ccc(N(c3ccc4c(c3)C3(CCCCC3)c3ccccc3-4)c3ccccc3-c3ccccc3-c3ccccc3-c3ccccc3)cc2)cc1 ; CC1(C)c2ccccc2-c2ccc(N(c3ccc(-c4ccccc4)cc3)c3ccccc3-c3ccccc3-c3ccccc3-c3ccccc3)cc21]

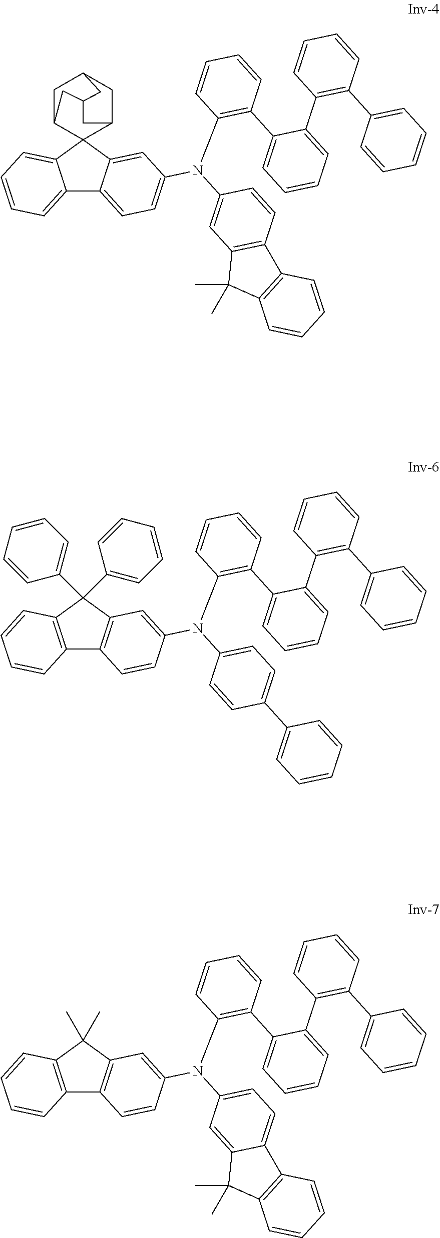 [SMILES: CC1(C)c2ccccc2-c2ccc(N(c3ccc4c(c3)C3(c5ccccc5-4)C4CC5CC(C4)CC3C5)c3ccccc3-c3ccccc3-c3ccccc3-c3ccccc3)cc21 ; c1ccc(-c2ccc(N(c3ccc4c(c3)C(c3ccccc3)(c3ccccc3)c3ccccc3-4)c3ccccc3-c3ccccc3-c3ccccc3-c3ccccc3)cc2)cc1 ; CC1(C)c2ccccc2-c2ccc(N(c3ccc4c(c3)C(C)(C)c3ccccc3-4)c3ccccc3-c3ccccc3-c3ccccc3-c3ccccc3)cc21]

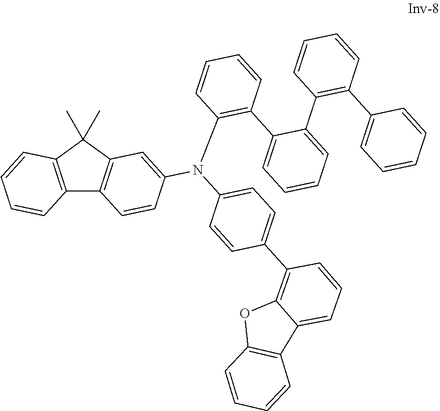 [SMILES: CC1(C)c2ccccc2-c2ccc(N(c3ccc(-c4cccc5c4oc4ccccc45)cc3)c3ccccc3-c3ccccc3-c3ccccc3-c3ccccc3)cc21]

Comparative compounds used for producing the organic EL devices of Comparative examples 1 to 3

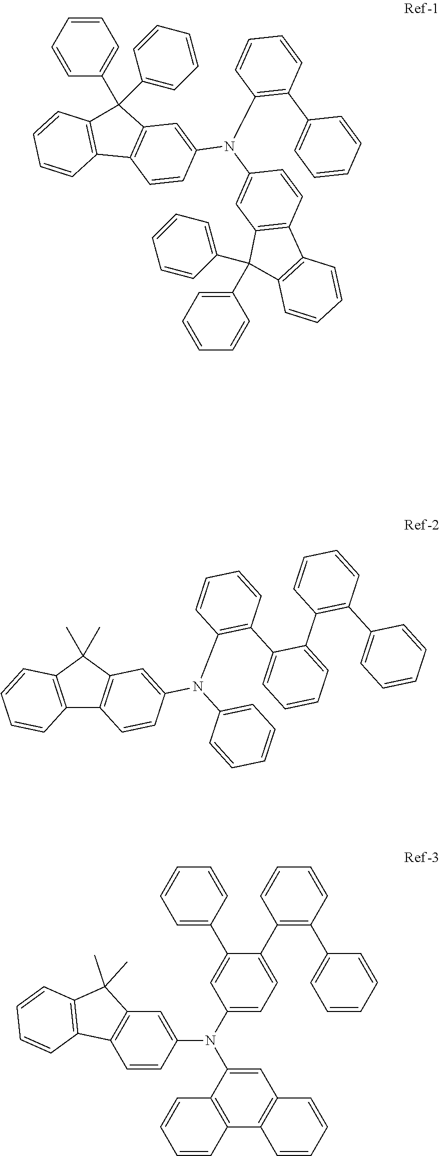 [SMILES: c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1 ; CC1(C)c2ccccc2-c2ccc(N(c3ccccc3)c3ccccc3-c3ccccc3-c3ccccc3-c3ccccc3)cc21 ; CC1(C)c2ccccc2-c2ccc(N(c3ccc(-c4ccccc4-c4ccccc4)c(-c4ccccc4)c3)c3cc4ccccc4c4ccccc34)cc21]

Other compounds used for producing the organic EL devices of Examples 1 to 7 and the Comparative examples 1 to 3

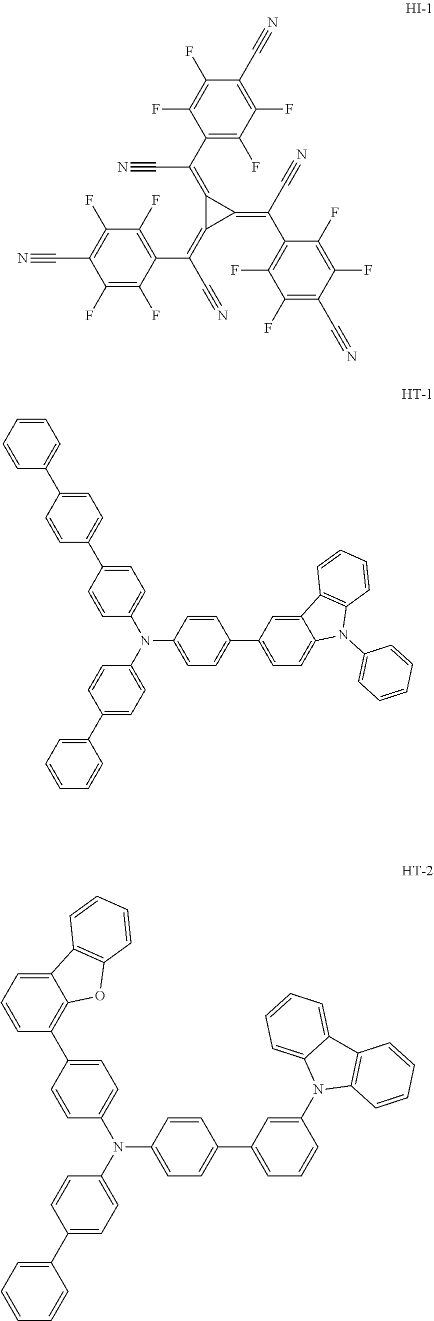 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; c1ccc(-c2ccc(-c3ccc(N(c4ccc(-c5ccccc5)cc4)c4ccc(-c5ccc6c(c5)c5ccccc5n6-c5ccccc5)cc4)cc3)cc2)cc1 ; c1ccc(-c2ccc(N(c3ccc(-c4cccc(-n5c6ccccc6c6ccccc65)c4)cc3)c3ccc(-c4cccc5c4oc4ccccc45)cc3)cc2)cc1]

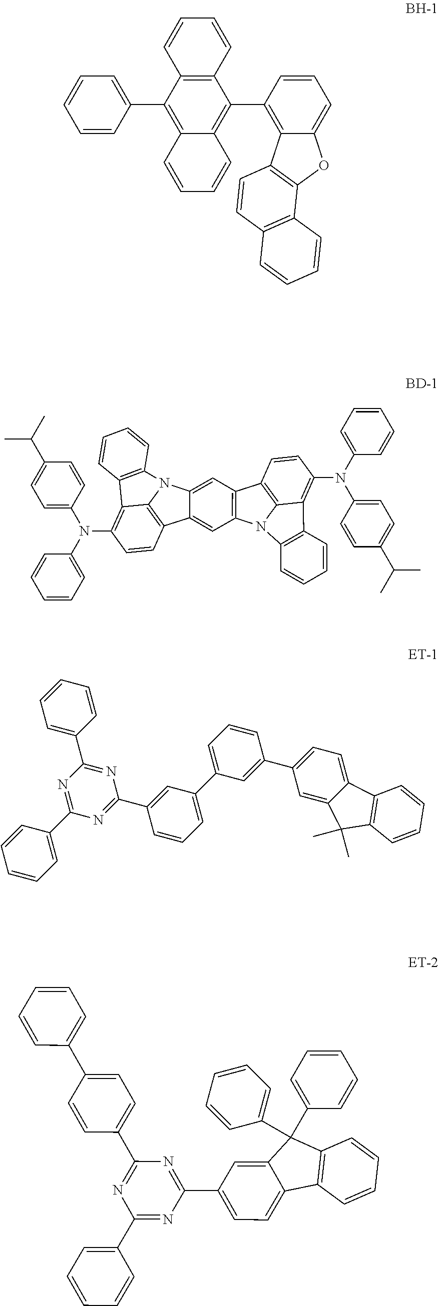 [SMILES: c1ccc(-c2c3ccccc3c(-c3cccc4oc5c6ccccc6ccc5c34)c3ccccc23)cc1 ; CC(C)c1ccc(N(c2ccccc2)c2ccc3c4cc5c(cc4n4c6ccccc6c2c34)c2ccc(N(c3ccccc3)c3ccc(C(C)C)cc3)c3c4ccccc4n5c23)cc1 ; CC1(C)c2ccccc2-c2ccc(-c3cccc(-c4cccc(-c5nc(-c6ccccc6)nc(-c6ccccc6)n5)c4)c3)cc21 ; c1ccc(-c2ccc(-c3nc(-c4ccccc4)nc(-c4ccc5c(c4)C(c4ccccc4)(c4ccccc4)c4ccccc4-5)n3)cc2)cc1]

### Preparation of Organic EL Device

### Example 1

A 25 mm×75 mm×1.1 mm glass substrate (manufactured by Geomatec Co., Ltd.) with an ITO transparent electrode (anode) was ultrasonically washed in isopropyl alcohol for 5 minutes and then UV ozone washed for 30 minutes. The film thickness of ITO was 130 nm.

The glass substrate with an ITO transparent electrode after washing was mounted on a substrate holder of a vacuum evaporation device.

First, a compound HT-1 and a compound HI-1 were vapor co-deposited onto the surface on which the transparent electrode was formed so as to cover the transparent electrode, to form a hole injecting layer with a film thickness of 10 nm. The mass ratio of the compound HT-1 and the compound HI-1 (HT-1: HI-1) was 97:3.

Next, the compound HT-1 was vapor-deposited on the hole injecting layer to form a first hole transporting layer with a film thickness of 40 nm.

Next, the inventive compound Inv-1 was vapor-deposited on the first hole transporting layer to form a second hole transporting layer with a film thickness of 40 nm.

Next, the compound HT-2 was vapor-deposited on the second hole transporting layer to form a third hole transporting layer with a film thickness of 5 nm.

Next, a compound BH-1 (host material) and a compound BD-1 (dopant material) were vapor co-deposited onto the third hole transporting layer to form a light emitting layer with a film thickness of 20 nm. The mass ratio of the compound BH-1 and the compound BD-1 (BH-1: BD-1) was 99:1.

Next, on the light emitting layer, compound ET-1 was vapor-deposited to form a first electron transporting layer with a film thickness of 5 nm.

Next, the compound ET-2 and Liq were vapor co-deposited on the first electron transporting layer to form a second electron transporting layer with a film thickness of 25 nm. The mass ratio of compound ET-2 and Liq (ET-2: Liq) was 50:50.

Next, Yb was vapor-deposited on the second electron transporting layer to form an electron injecting electrode having a film thickness of 1 nm.

Then, metal Al was vapor-deposited on this electron injecting electrode to form a metal cathode with a film thickness of 50 nm.

The layer configuration of the organic EL device obtained by this manner is shown below.

ITO (130)/HT-1:HI-1=97:3 (10)/HT-1 (40)/Inv-1 (40)/HT-2 (5)/BH-1:BD-1=99:1 (20)/ET-1 (5)/ET-2:Liq=50:50 (25)/Yb (1)/Al (50)

In the above layer configuration, the numbers in parentheses are film thicknesses (nm), and the ratios are mass ratios.

### Comparative Example 1

An organic EL device was prepared in the same manner as in Example 1, except that the comparative compound Ref-1 was used instead of inventive compound Inv-1.

### Example 2

An organic EL device was prepared in the same manner as in Example 1, except that the inventive compound Inv-2 was used instead of inventive compound Inv-1.

### Example 3

An organic EL device was prepared in the same manner as in Example 1, except that the inventive compound Inv-3 was used instead of inventive compound Inv-1.

### Example 4

An organic EL device was prepared in the same manner as in Example 1, except that the inventive compound Inv-4 was used instead of inventive compound Inv-1.

### Example 5

An organic EL device was prepared in the same manner as in Example 1, except that the inventive compound Inv-6 was used instead of inventive compound Inv-1.

### Example 6

An organic EL device was prepared in the same manner as in Example 1, except that the inventive compound Inv-7 was used instead of inventive compound Inv-1.

### Example 7

An organic EL device was prepared in the same manner as in Example 1, except that the inventive compound Inv-8 was used instead of inventive compound Inv-1.

### Comparative Example 2

An organic EL device was prepared in the same manner as in Example 1, except that the comparative compound Ref-2 was used instead of inventive compound Inv-1.

### Comparative Example 3

An organic EL device was prepared in the same manner as in Example 1, except that the comparative compound Ref-3 was used instead of inventive compound Inv-1.

### Evaluation of Organic EL Devices

### (1) Measurement of Drive Voltage

The voltage (V) was measured when a voltage was applied to the organic EL device so that the current density was 10 mA/cm². The results are shown in Table 1.

### (2) Measurement of External Quantum Efficiency (EQE)

The obtained organic EL device was driven at constant DC current at a current density of 10 mA/cm² at room temperature. Luminance was measured using a luminance meter (Spectroluminance radiometer CS-1000 manufactured by Minolta Co., Ltd.), and the external quantum efficiency (%) was determined from the results. The results are shown in Table 1.

TABLE 1

| | Second hole | | External quantum |
|---|---|---|---|
| | transporting | Drive voltage (V) | efficiency (%) |
| | layer material | @ 10 mA/cm² | @ 10 mA/cm² |
| Example 1 | Inv-1 | 3.28 | 12.3 |
| Comparative | Ref-1 | 3.43 | 10.2 |
| example 1 | | | |
| Example 2 | Inv-2 | 3.27 | 12.4 |
| Example 3 | Inv-3 | 3.21 | 12.3 |
| Example 4 | Inv-4 | 3.22 | 12.4 |
| Example 5 | Inv-6 | 3.21 | 12.2 |
| Example 6 | Inv-7 | 3.21 | 12.2 |
| Example 7 | Inv-8 | 3.25 | 12.0 |
| Comparative | Ref-2 | 3.33 | 10.6 |
| example 2 | | | |
| Comparative | Ref-3 | 3.45 | 10.1 |
| example 3 | | | |

As is clear from the results in Table 1, the inventive compounds Inv-1 to Inv-4 and Inv-6 to Inv-8 provide an organic EL device which is driven at a lower voltage and has higher external quantum efficiency than the comparative compounds Ref−1 to Ref-3.

### Inventive Compound Synthesized in Synthesis Example

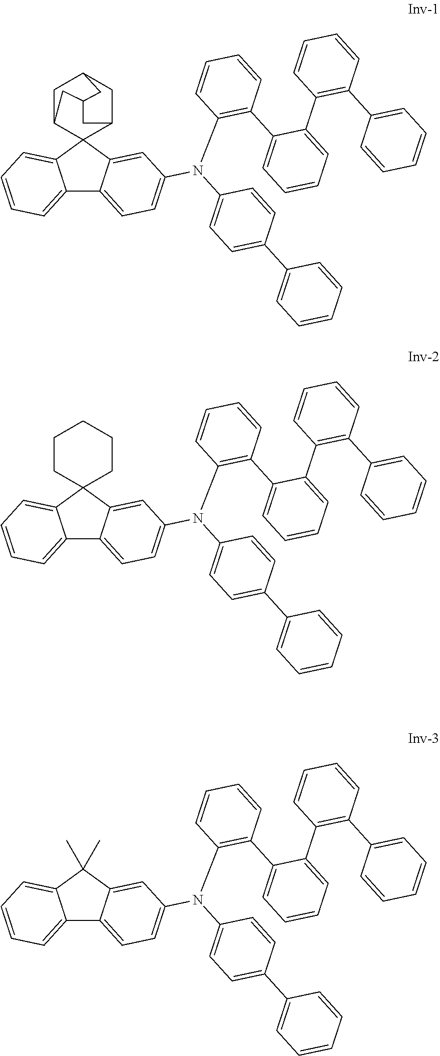 [SMILES: c1ccc(-c2ccc(N(c3ccc4c(c3)C3(c5ccccc5-4)C4CC5CC(C4)CC3C5)c3ccccc3-c3ccccc3-c3ccccc3-c3ccccc3)cc2)cc1 ; c1ccc(-c2ccc(N(c3ccc4c(c3)C3(CCCCC3)c3ccccc3-4)c3ccccc3-c3ccccc3-c3ccccc3-c3ccccc3)cc2)cc1 ; CC1(C)c2ccccc2-c2ccc(N(c3ccc(-c4ccccc4)cc3)c3ccccc3-c3ccccc3-c3ccccc3-c3ccccc3)cc21]

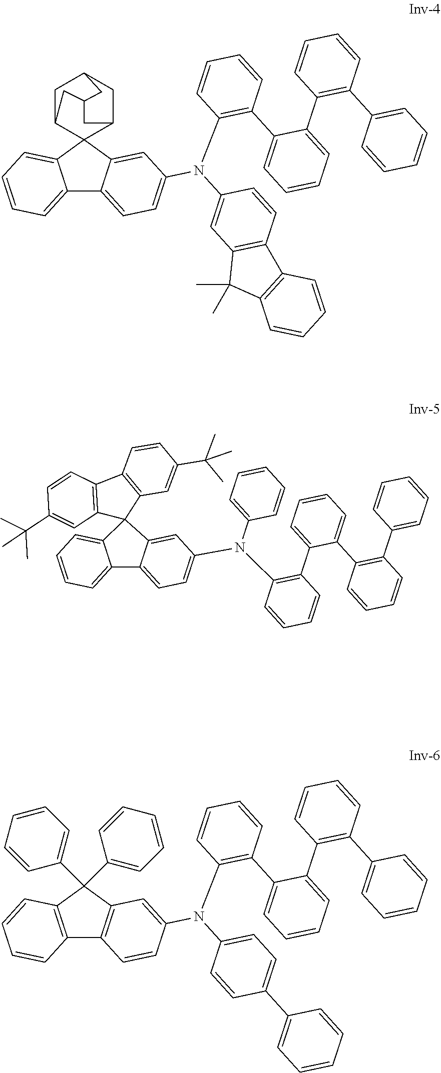 [SMILES: CC1(C)c2ccccc2-c2ccc(N(c3ccc4c(c3)C3(c5ccccc5-4)C4CC5CC(C4)CC3C5)c3ccccc3-c3ccccc3-c3ccccc3-c3ccccc3)cc21 ; CC(C)(C)c1ccc2c(c1)C1(c3ccccc3-c3ccc(N(c4ccccc4)c4ccccc4-c4ccccc4-c4ccccc4-c4ccccc4)cc31)c1cc(C(C)(C)C)ccc1-2 ; c1ccc(-c2ccc(N(c3ccc4c(c3)C(c3ccccc3)(c3ccccc3)c3ccccc3-4)c3ccccc3-c3ccccc3-c3ccccc3-c3ccccc3)cc2)cc1]

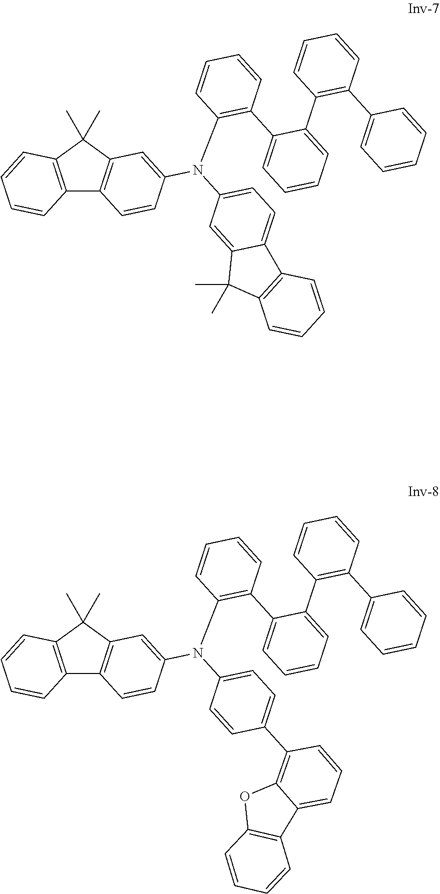 [SMILES: CC1(C)c2ccccc2-c2ccc(N(c3ccc4c(c3)C(C)(C)c3ccccc3-4)c3ccccc3-c3ccccc3-c3ccccc3-c3ccccc3)cc21 ; CC1(C)c2ccccc2-c2ccc(N(c3ccc(-c4cccc5c4oc4ccccc45)cc3)c3ccccc3-c3ccccc3-c3ccccc3-c3ccccc3)cc21]

### Intermediate Synthesis Example 1: Synthesis of Intermediate A

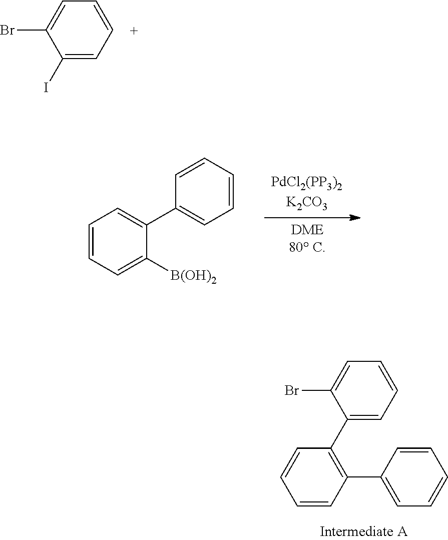 [SMILES: Brc1ccccc1I ; OB(O)c1ccccc1-c1ccccc1 ; Brc1ccccc1-c1ccccc1-c1ccccc1]

Under an argon atmosphere, a mixture of 1-bromo-2-iodobenzene (11.37 g, 40.2 mmol), 2-biphenylboronic acid (6.58 g, 33.2 mmol), bis(triphenylphosphine)palladium(II) dichloride (1.16 G, 1.66 mmol), sodium carbonate (19.02 g, 179 mmol), DME (200 mL), ethanol (8 mL) and water (90 mL) was stirred at 80° C. for 7 hours. The reaction solution was cooled to room temperature and concentrated under reduced pressure. The obtained residue was purified by silica gel column chromatography to obtain Intermediate A as a colorless liquid (8.05 g). The yield was 78%.

### Intermediate Synthesis Example 2: Synthesis of Intermediate B

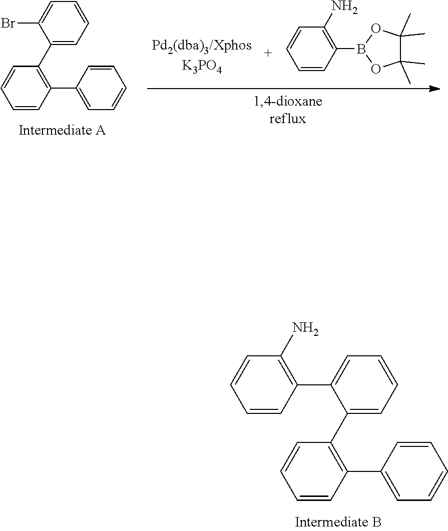 [SMILES: Brc1ccccc1-c1ccccc1-c1ccccc1 ; CC1(C)OB(c2ccccc2N)OC1(C)C ; Nc1ccccc1-c1ccccc1-c1ccccc1-c1ccccc1]

A mixture of the obtained intermediate A (8.05 g, 26 mmol), 2-(4,4,5,5-tetramethyl-1,3,2-dioxaborolan-2-yl) aniline (5.99 g, 27.3 mmol), tris(dibenzylideneacetone)dipalladium(0) (0.238 g, 0.26 mmol), 2-dicyclohexylphosphino-2′,4′,6′-triisopropylbiphenyl (Xphos) (0.496 g, 1.04 mmol), tripotassium phosphate (16.58 g, 78 mmol), 1,4-dioxane (174 mL) and water (39 mL) was stirred at 100° C. for 5 hours under an argon atmosphere. The reaction solution was cooled to room temperature, water was added, and then concentrated under reduced pressure. The obtained residue was purified by silica gel column chromatography to obtain Intermediate B as a pale yellow solid (8.28 g). The yield was 99%.

### Intermediate Synthesis Example 3: Synthesis of Intermediate C

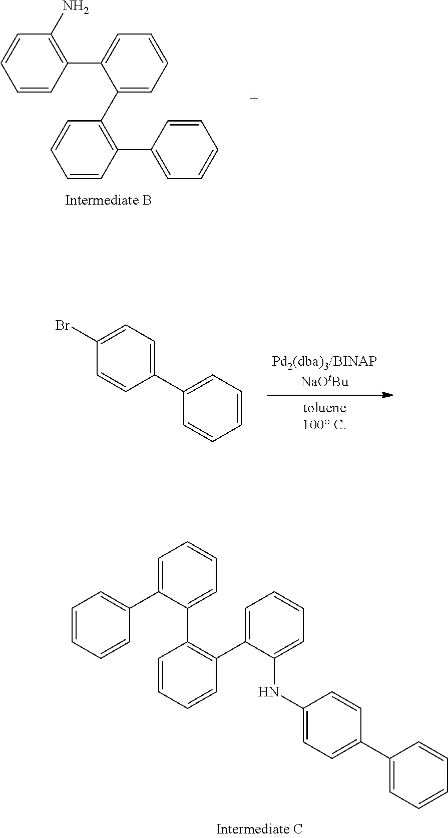 [SMILES: Nc1ccccc1-c1ccccc1-c1ccccc1-c1ccccc1 ; Brc1ccc(-c2ccccc2)cc1 ; c1ccc(-c2ccc(Nc3ccccc3-c3ccccc3-c3ccccc3-c3ccccc3)cc2)cc1]

Under argon atmosphere, a mixture of the intermediate B (3.37 g, 10.48 mmol), 4-bromobiphenyl (2.44 g, 10.48 mmol), tris(dibenzylideneacetone)dipalladium(0) (0.192 g, 0.210 mmol)), BINAP (0.261 g, 0.419 mmol), sodium-t-butoxide (1.41 g, 14.68 mmol), and toluene (70 mL) were stirred at 100° C. for 3 hours. The reaction solution was cooled to room temperature and concentrated under reduced pressure. The obtained residue was purified by silica gel column chromatography and recrystallization to obtain 4.66 g of Intermediate C as a white solid. The yield was 94%.

### Intermediate Synthesis Example 4: Synthesis of Intermediate D

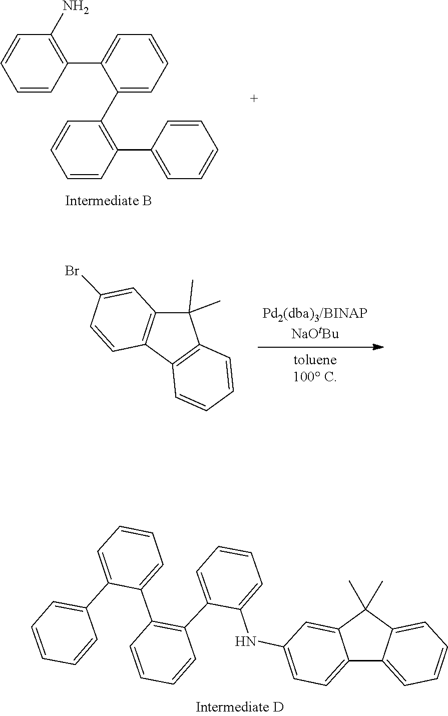 [SMILES: Nc1ccccc1-c1ccccc1-c1ccccc1-c1ccccc1 ; CC1(C)c2ccccc2-c2ccc(Br)cc21 ; CC1(C)c2ccccc2-c2ccc(Nc3ccccc3-c3ccccc3-c3ccccc3-c3ccccc3)cc21]

The intermediate D was obtained by performing the same operation as in Intermediate Synthesis Example 3, except that 2-bromo-9,9-dimethyl-9H-fluorene was used instead of 4-bromobiphenyl used in Intermediate synthesis example 3. The yield was 91%.

### Intermediate Synthesis Example 5: Synthesis of Intermediate E

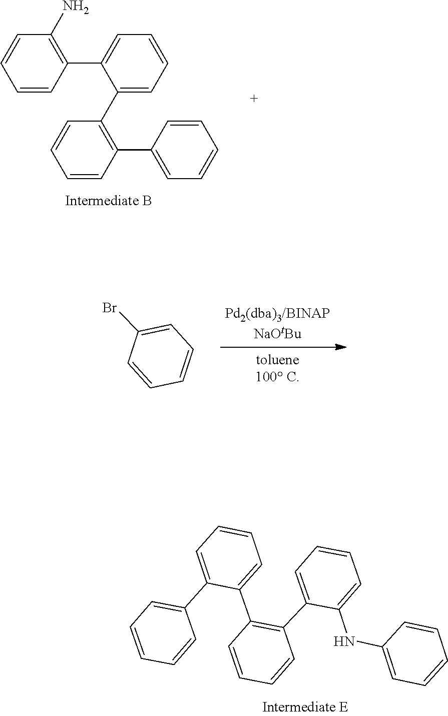 [SMILES: Nc1ccccc1-c1ccccc1-c1ccccc1-c1ccccc1 ; Brc1ccccc1 ; c1ccc(Nc2ccccc2-c2ccccc2-c2ccccc2-c2ccccc2)cc1]

The intermediate E was obtained by performing the same procedure as in Intermediate synthesis example 3, except that bromobenzene was used instead of 4-bromobiphenyl used in Intermediate synthesis example 3. The yield was 91%.

### Synthesis Example 1: Synthesis of Inventive Compound Inv-1

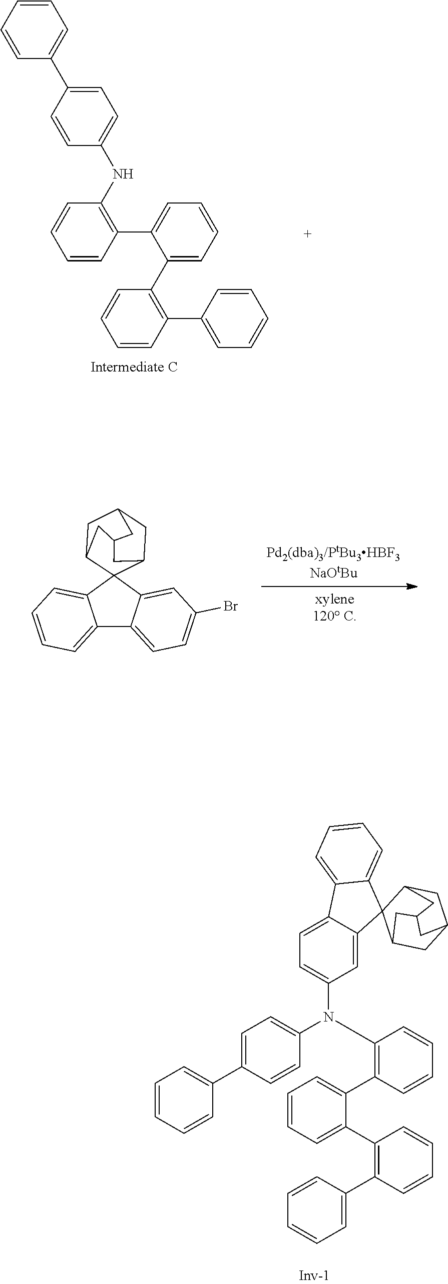 [SMILES: c1ccc(-c2ccc(Nc3ccccc3-c3ccccc3-c3ccccc3-c3ccccc3)cc2)cc1 ; Brc1ccc2c(c1)C1(c3ccccc3-2)C2CC3CC(C2)CC1C3 ; c1ccc(-c2ccc(N(c3ccc4c(c3)C3(c5ccccc5-4)C4CC5CC(C4)CC3C5)c3ccccc3-c3ccccc3-c3ccccc3-c3ccccc3)cc2)cc1]

A mixture of the intermediate C (3.8 g, 8.02 mmol), 2′-bromospiro[adamantane-2,9′-fluorene] (3.22 g, 8.83 mmol), tris(dibenzylideneacetone)dipalladium(0) (0.147 g, 0.160 mmol), tri-t-butylphosphonium tetrafluoroborate (0.186 g, 0.642 mmol), sodium-t-butoxide (1.079 g, 11.23 mmol), and xylene (54 mL) was stirred at 120° C. for 5 hours. The reaction solution was cooled to room temperature and concentrated under reduced pressure. The obtained residue was purified by silica gel column chromatography and recrystallization to obtain 3.08 g of Intermediate C as a white solid. The obtained white solid was compound Inv-1 as a result of mass spectrometry analysis (m/e=757 for molecular weight 757.37).

### Synthesis Example 2: Synthesis of Inventive Compound Inv-2

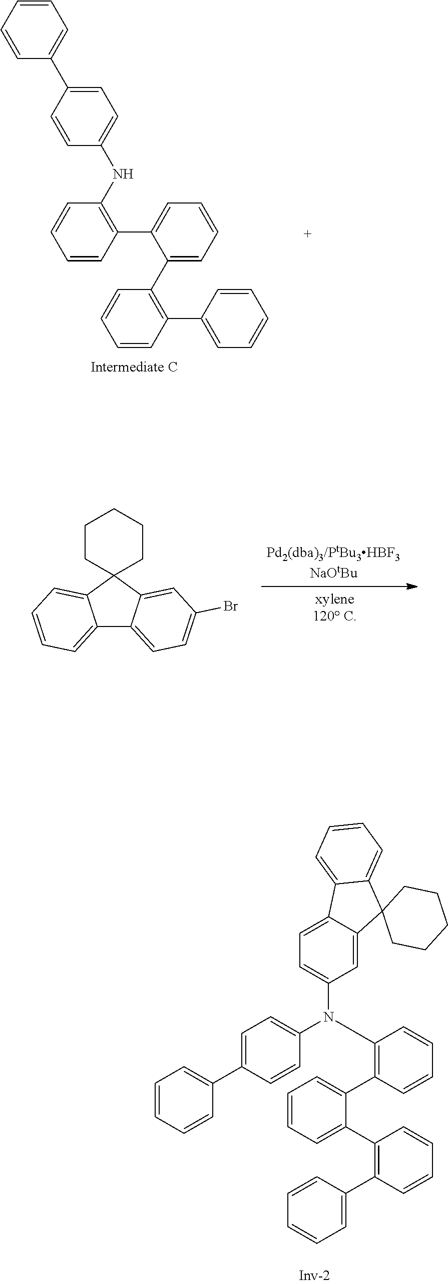 [SMILES: c1ccc(-c2ccc(Nc3ccccc3-c3ccccc3-c3ccccc3-c3ccccc3)cc2)cc1 ; Brc1ccc2c(c1)C1(CCCCC1)c1ccccc1-2 ; c1ccc(-c2ccc(N(c3ccc4c(c3)C3(CCCCC3)c3ccccc3-4)c3ccccc3-c3ccccc3-c3ccccc3-c3ccccc3)cc2)cc1]

A compound was obtained by performing the same operation as in Synthesis Example 1, except that 2′-bromospiro[cyclohexane-1,9′-fluorene] was used instead of 2′-bromospiro[adamantane-2,9′-fluorene] used in Synthesis Example 1. The obtained compound was the compound Inv-2 as a result of mass spectrometry analysis (m/e-705 for molecular weight 705.34).

### Synthesis Example 3: Synthesis of Inventive Compound Inv-3

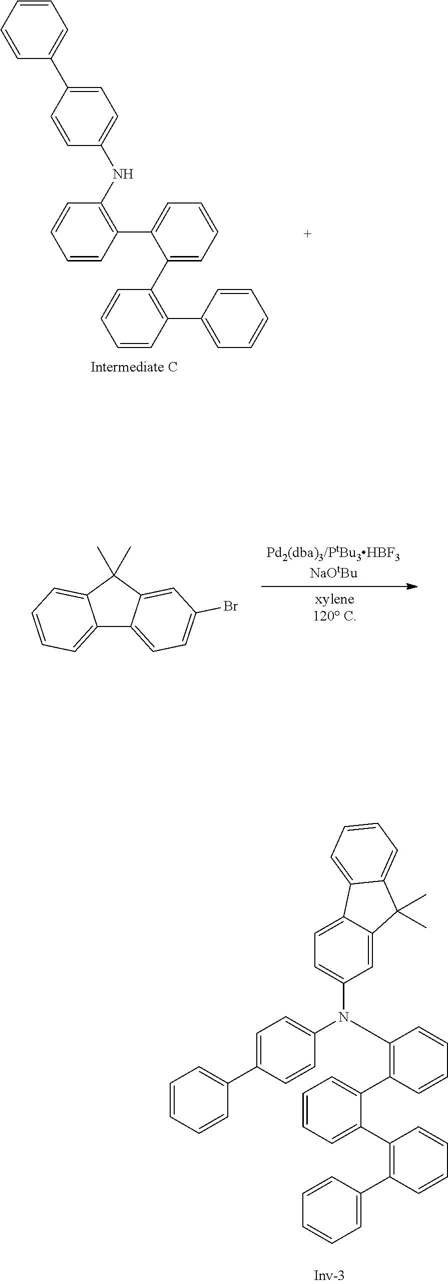 [SMILES: c1ccc(-c2ccc(Nc3ccccc3-c3ccccc3-c3ccccc3-c3ccccc3)cc2)cc1 ; CC1(C)c2ccccc2-c2ccc(Br)cc21 ; CC1(C)c2ccccc2-c2ccc(N(c3ccc(-c4ccccc4)cc3)c3ccccc3-c3ccccc3-c3ccccc3-c3ccccc3)cc21]

A compound was obtained by performing the same operation as in Synthesis Example 1, except that 2-bromo-9,9-dimethyl-9H-fluorene was used instead of 2′-bromospiro[adamantane-2,9′-fluorene] used in Synthesis Example 1. The obtained compound was the compound Inv-3 as a result of mass spectrometry analysis (m/e-665 for molecular weight 665.31).

### Synthesis Example 4: Synthesis of Inventive Compound Inv-4

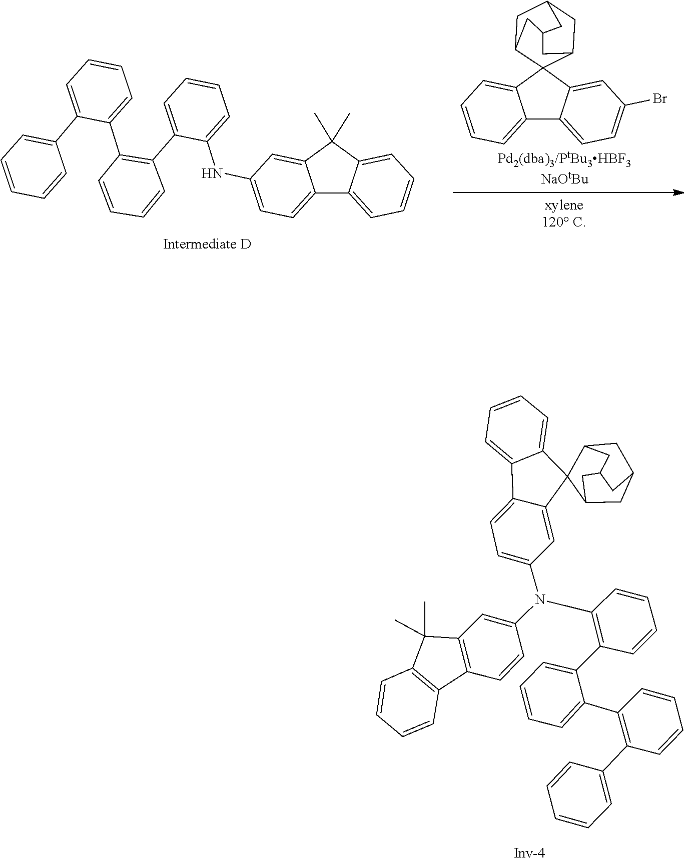 [SMILES: CC1(C)c2ccccc2-c2ccc(Nc3ccccc3-c3ccccc3-c3ccccc3-c3ccccc3)cc21 ; Brc1ccc2c(c1)C1(c3ccccc3-2)C2CC3CC(C2)CC1C3 ; CC1(C)c2ccccc2-c2ccc(N(c3ccc4c(c3)C3(c5ccccc5-4)C4CC5CC(C4)CC3C5)c3ccccc3-c3ccccc3-c3ccccc3-c3ccccc3)cc21]

A compound was obtained by performing the same operation as in Synthesis Example 1 except that Intermediate D was used in place of Intermediate C used in Synthesis Example 1. The obtained compound was the compound Inv-4 as a result of mass spectrometry analysis (m/e-797 for molecular weight 797.40).

### Synthesis Example 5: Synthesis of Inventive Compound Inv-5

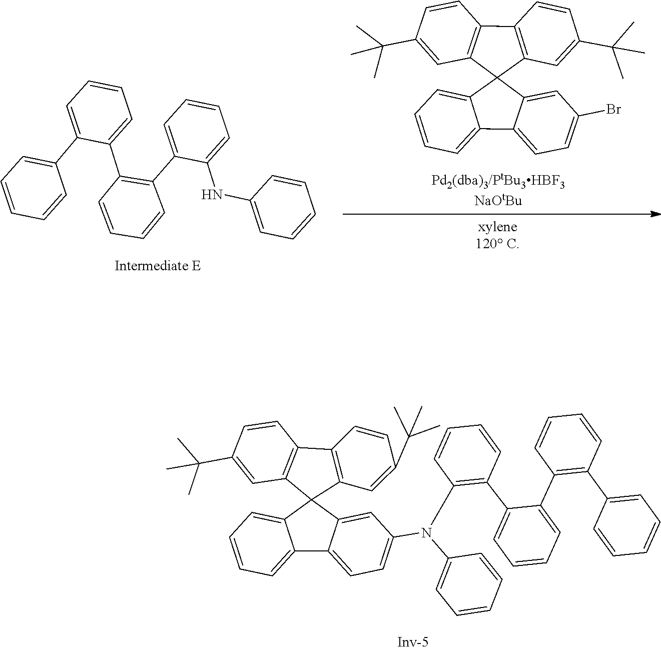

A compound was obtained by performing the same operation as in Synthesis Example 1, except that the intermediate E was used instead of the intermediate C used in Synthesis Example 1, and 2′-bromo-2,7-di-tert-butyl-9,9′-spirobi[fluorene] instead of 2′-bromospiro[adamantane-2,9′-fluorene]. The obtained compound was compound Inv-5 as a result of mass spectrometry analysis (m/e-823 for molecular weight 823.42).

### Synthesis Example 6: Synthesis of Inventive Compound Inv-6

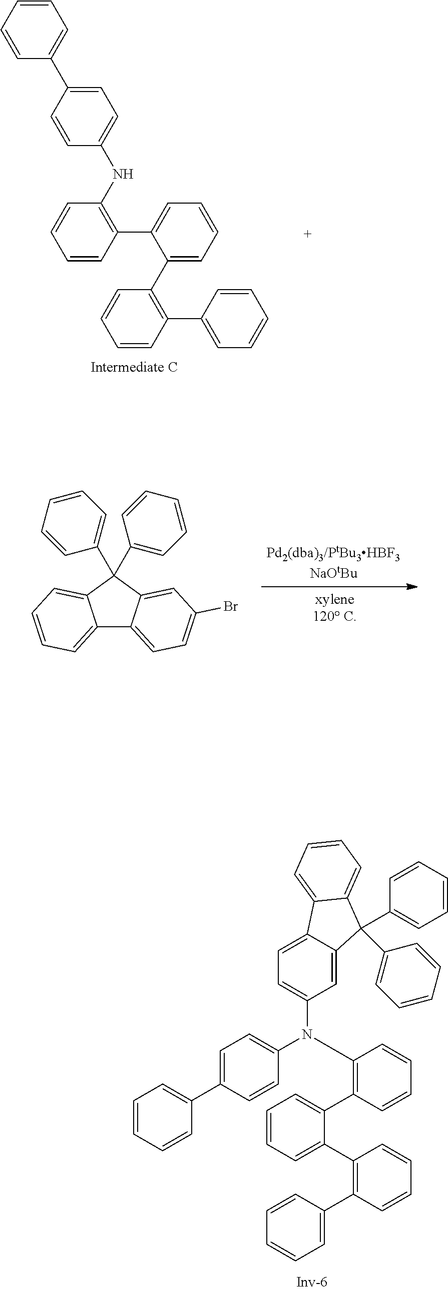 [SMILES: c1ccc(-c2ccc(Nc3ccccc3-c3ccccc3-c3ccccc3-c3ccccc3)cc2)cc1 ; Brc1ccc2c(c1)C(c1ccccc1)(c1ccccc1)c1ccccc1-2 ; c1ccc(-c2ccc(N(c3ccc4c(c3)C(c3ccccc3)(c3ccccc3)c3ccccc3-4)c3ccccc3-c3ccccc3-c3ccccc3-c3ccccc3)cc2)cc1]

A compound was obtained by performing the same operation as in Synthesis Example 1, except that 2-bromo-9,9-diphenylfluorene was used instead of 2′-bromospiro[adamantane-2,9′-fluorene] used in Synthesis Example 1. The obtained compound was compound Inv-6 as a result of mass spectrometry analysis (m/e=790 for molecular weight 790.02).

### Synthesis Example 7: Synthesis of Inventive Compound Inv-7

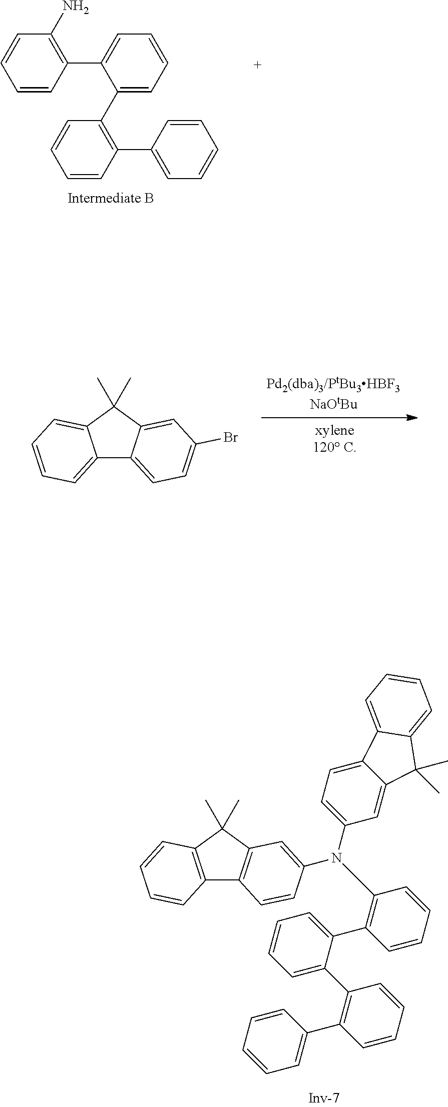 [SMILES: Nc1ccccc1-c1ccccc1-c1ccccc1-c1ccccc1 ; CC1(C)c2ccccc2-c2ccc(Br)cc21 ; CC1(C)c2ccccc2-c2ccc(N(c3ccc4c(c3)C(C)(C)c3ccccc3-4)c3ccccc3-c3ccccc3-c3ccccc3-c3ccccc3)cc21]

A mixture of the intermediate B (2.7 g, 8.4 mmol), 2-bromo-9,9-dimethylfluorene (5.736 g, 21 mmol), tris(dibenzylideneacetone)dipalladium(0) (0.154 g, 0.168 mmol), tri-t-butylphosphonium tetrafluoroborate (0.195 g, 0.672 mmol), sodium-t-butoxide (2.260 g, 23.52 mmol), and xylene (56 mL) was stirred at 120° C. for 5 hours. The reaction solution was cooled to room temperature and concentrated under reduced pressure. The obtained residue was purified by silica gel column chromatography and recrystallization to obtain 4.03 g of a white solid. The yield was 68%.

As a result of mass spectrometry analysis (m/e=705 for molecular weight 705.34), the obtained product was compound Inv-7.

### Synthesis Example 8: Synthesis of Inventive Compound Inv-8

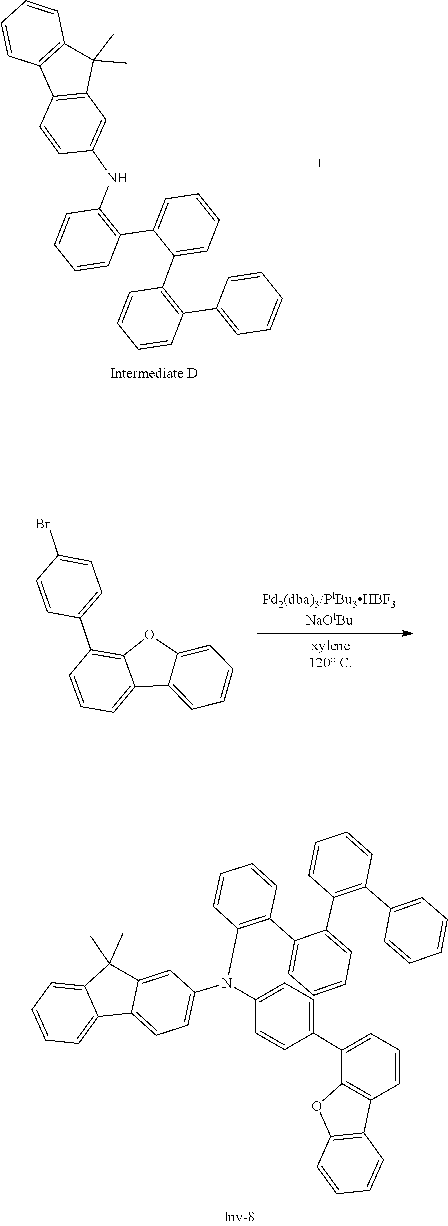 [SMILES: CC1(C)c2ccccc2-c2ccc(Nc3ccccc3-c3ccccc3-c3ccccc3-c3ccccc3)cc21 ; Brc1ccc(-c2cccc3c2oc2ccccc23)cc1 ; CC1(C)c2ccccc2-c2ccc(N(c3ccc(-c4cccc5c4oc4ccccc45)cc3)c3ccccc3-c3ccccc3-c3ccccc3-c3ccccc3)cc21]

A compound was obtained by performing the same operation as in Synthesis Example 4, except that 4-(4-bromophenyl)dibenzofuran was used instead of 2′-bromospiro[adamantane-2,9′-fluorene] used in Synthesis Example 4. The obtained compound was the compound Inv-8 as a result of mass spectrometry analysis (m/e=755 for molecular weight 755.96).

### REFERENCE SIGNS LIST

1, 11, 12 Organic EL device
2 Substrate
3 Anode
4 Cathode
5 Light emitting layer
5a First light emitting layer
5b Second light emitting layer
6 Hole transporting band (Hole transporting layer)
6a Hole injecting layer
6b First hole transporting layer material
6c Second hole transporting layer material
6d Third hole transporting layer material
7 Electron transporting band (Electron transporting layer)
7a First electron transporting layer
7b Second electron transporting layer
10, 20, 30 Light emitting unit

1. A compound represented by the following formula (1), 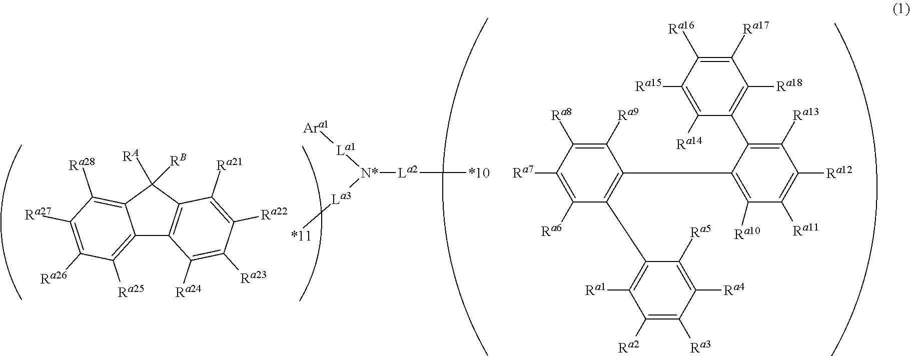
wherein
N* is a central nitrogen atom,
L^{a1} is a substituted or an unsubstituted phenylene group, a substituted or an unsubstituted naphthylene group, or a substituted or an unsubstituted biphenylene group, and a substituent is an unsubstituted alkyl group comprising 1 to 6 carbon atoms or an unsubstituted aryl group comprising 6 to 12 ring carbon atoms, and no ring is condensed with the phenylene group, the biphenylene group, and the naphthylene group,
L^{a2} and L^{a3} are each independently a single bond, a substituted or an unsubstituted phenylene group, a substituted or an unsubstituted naphthylene group, or a substituted or an unsubstituted biphenylene group, and a substituent is an unsubstituted alkyl group comprising 1 to 6 carbon atoms or an unsubstituted aryl group comprising 6 to 12 ring carbon atoms, and no ring is condensed with the phenylene group, the biphenylene group, and the naphthylene group,
one selected from R^{a1} to R^{a5} is a single bond bonded to *10, and R^{a1} to R^{a5} which are not a single bond bonded to *10, and R^{a6} to R^{a18} are a hydrogen atom,
provided that two adjacent ones selected from R^{a1} to R^{a5} which are not a single bond bonded to *10 and R^{a6} to R^{a18} are bonded to each other to form a benzene ring, or R^{a6} to R^{a18} are not bonded to each other and therefore do not form a ring,
Ar^{a1} is a substituted or an unsubstituted aryl group comprising 6 to 30 ring carbon atoms, a substituted or an unsubstituted aromatic heterocyclic group comprising 5 to 30 ring atoms, or a triarylsilyl group comprising an aryl group having 6 to 30 ring carbon atoms,
one selected from R^{a21} to R^{a24} is a single bond bonded to *11, and *R^{a21} to R^{a24} which are not a single bond bonded to *11, and R^{a25} to R^{a28} are each independently a hydrogen atom, a substituted or an unsubstituted alkyl group comprising 1 to 30 carbon atoms, or a substituted or an unsubstituted aryl group comprising 6 to 30 ring carbon atoms, or a substituted or an unsubstituted aromatic heterocyclic group comprising 5 to 30 ring atoms,
provided that two adjacent ones selected from R^{a21} to R^{a24} which are not a single bond bonded to *11 and R^{a25} to R^{a28} are bonded to each other to form a benzene ring, or R^{a25} to R^{a28} are not bonded to each other and therefore do not form a ring,
R^{A} and R^{B} are each independently a substituted or an unsubstituted alkyl group comprising 1 to 30 carbon atoms, a substituted or an unsubstituted aryl group comprising 6 to 30 ring carbon atoms, or a substituted or an unsubstituted aromatic heterocyclic group comprising 5 to 30 ring atoms, and
provided that R^{A} and R^{B} are bonded to each other to form a substituted or an unsubstituted spiro ring with the carbon atom at the 9-position of the fluorene skeleton, or R^{A} and R^{B} are not bonded to each other and do not form a spiro ring.

2. The compound of claim 1, wherein at least one of R^{A} and R^{B} is a substituted or an unsubstituted alkyl group comprising 1 to 30 carbon atoms.

3. The compound of claim 1, wherein R^{A} and the R^{B} form the spiro ring.

4. The compound of claim 3, wherein the spiro ring is selected from the group consisting of

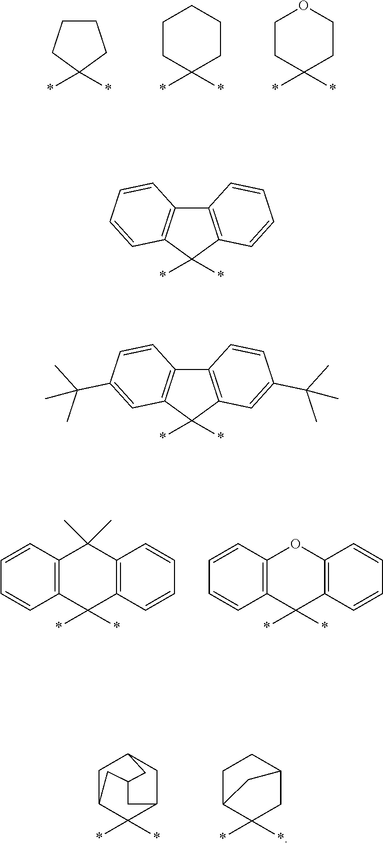

5. The compound of claim 1, wherein one selected from R^{a22} to R^{a27} is a single bond bonded to *11.

6-9. (canceled)

10. The compound of claim 1, wherein Ar^{a1} is represented by the following formula (1a), (1c), (1d), (1e), or (1f), 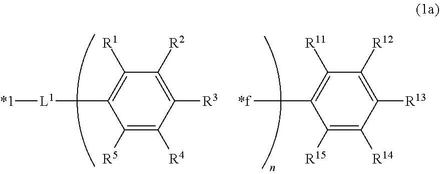
wherein,
*1 is bonded to L^{a1},
L¹ is a single bond or an unsubstituted arylene group comprising 6 to 12 ring carbon atoms,
R¹ to R⁵ are each independently a hydrogen atom, a substituted or an unsubstituted alkyl group comprising 1 to 6 carbon atoms, or a substituted or an unsubstituted aryl group comprising 6 to 12 ring carbon atoms,
provided that one selected from R¹ to R⁵ is a single bond bonded to *f,
two adjacent ones selected from R¹ to R⁵ which are not a single bond bonded to *f are not bonded to each other and therefore do not form a ring structure,
n is 0 or 1, and when n is 0, *f is bonded to L¹,
R¹¹ to R¹⁵ are each independently a hydrogen atom, a substituted or an unsubstituted alkyl group comprising 1 to 6 carbon atoms, or a substituted or an unsubstituted aryl group comprising 6 to 12 ring carbon atoms, and
two adjacent ones selected from R¹¹ to R¹⁵ are not bonded to each other and therefore do not form a ring structure, 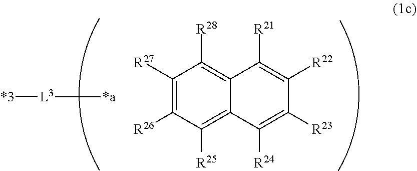
wherein,
*3 is bonded to L^{a1},
L³ is a single bond or an unsubstituted arylene group comprising 6 to 12 ring carbon atoms,
R²¹ to R²⁸ are each independently a hydrogen atom, a substituted or an unsubstituted alkyl group comprising 1 to 6 carbon atoms, or a substituted or an unsubstituted aryl group comprising 6 to 12 ring carbon atoms,
provided that one selected from R²¹ to R²⁸ is a single bond bonded to *a, and
two adjacent ones selected from R²¹ to R²⁸ which are not single bonds are not bonded to each other and therefore do not form a ring structure, 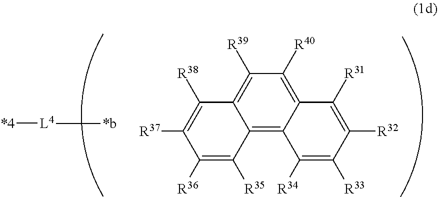
wherein,
*4 is bonded to L^{a1},
L⁴ is a single bond or an unsubstituted arylene group comprising 6 to 12 ring carbon atoms,
R³¹ to R⁴⁰ are each independently a hydrogen atom, a substituted or an unsubstituted alkyl group comprising 1 to 6 carbon atoms, or a substituted or an unsubstituted aryl group comprising 6 to 12 ring carbon atoms,
provided that one selected from R³¹ to R⁴⁰ is a single bond bonded to *b, and
two adjacent ones selected from R³¹ to R⁴⁰ which are not single bonds are not bonded to each other and therefore do not form a ring structure, 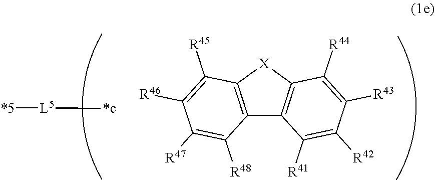
wherein,
*5 is bonded to L^{a1},
L⁵ is a single bond or an unsubstituted arylene group comprising 6 to 12 ring carbon atoms,
X is an oxygen atom, a sulfur atom, NRa, or CRbRc,
Ra is a hydrogen atom, a substituted or an unsubstituted alkyl group comprising 1 to 6 carbon atoms, a substituted or an unsubstituted aryl group comprising 6 to 12 ring atoms, or a substituted or an unsubstituted aromatic heterocyclic group comprising 5 to 13 ring atoms,
Rb is a substituted or an unsubstituted aryl group comprising 6 to 12 ring carbon atoms,
Rc is a substituted or an unsubstituted alkyl group comprising 1 to 6 carbon atoms or a substituted or an unsubstituted aryl group comprising 6 to 12 ring carbon atoms,
Rb and Rc are optionally bonded to each other to form a spiro ring structure,
R⁴¹ to R⁴⁸ are each independently a hydrogen atom, a substituted or an unsubstituted alkyl group comprising 1 to 6 carbon atoms, a substituted or an unsubstituted aryl group comprising 6 to 12 ring carbon atoms, or a substituted or an unsubstituted aromatic heterocyclic group comprising 5 to 13 ring atoms,
provided that one selected from R⁴¹ to R⁴⁸ and Ra is a single bond bonded to *c, and
two adjacent ones selected from R⁴¹ to R⁴⁸ which are not single bonds are optionally bonded to each other to form one or plural substituted or unsubstituted benzene rings, 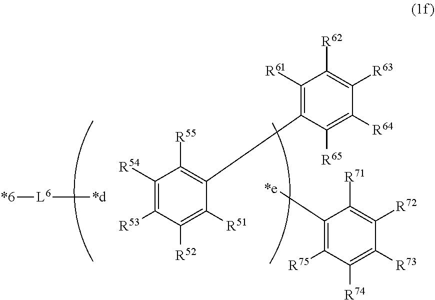
wherein,
*6 is bonded to L^{a1},
L⁶ is a single bond or an unsubstituted arylene comprising having 6 to 12 ring carbon atoms,
R⁵¹ to R⁵⁵ are each independently a hydrogen atom, an unsubstituted alkyl group comprising 1 to 6 carbon atoms, or an unsubstituted phenyl group,
provided that one selected from R⁵¹ to R⁵⁵ is a single bond bonded to *d, and the other selected from R⁵¹ to R⁵⁵ is a single bond bonded to *e,
two adjacent ones selected from R⁵¹ to R⁵⁵ which are neither a single bond bonded to *d nor a single bond bonded to *e are not bonded to each other and therefore do not form a ring structure,
R⁶¹ to R⁶⁵ and R⁷¹ to R⁷⁵ are each independently a hydrogen atom or an unsubstituted alkyl group comprising 1 to 6 carbon atoms, and
two adjacent ones selected from R⁶¹ to R⁶⁵ and R⁷¹ to R⁷⁵ are optionally bonded to each other to form one or plural substituted or unsubstituted benzene rings.

11. A compound represented by formula (2), 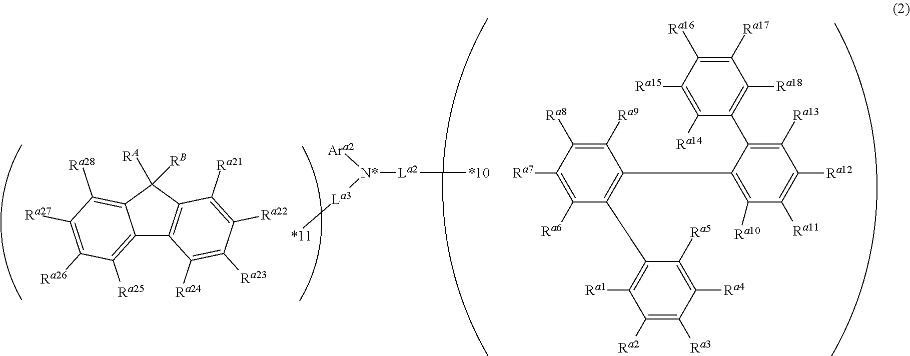
wherein
N* is a central nitrogen atom,
L^{a2}, L^{a3}, R^{a1} to R^{a5}, R^{a6} to R^{a18}, R^{a21} to R^{a24}, R^{a25} to R^{a28}, *10, *11, R^{A} and R^{B} are each as defined in formula (1), and
Ar^{a2} is a condensed aryl group comprising 14 to 30 ring carbon atoms which are substituted with an unsubstituted alkyl group comprising 1 to 30 carbon atoms or unsubstituted, a substituted or an unsubstituted condensed aromatic heterocyclic group comprising 13 to 30 ring atoms, or a substituted or an unsubstituted fluorenyl group.

12. The compound of claim 11, wherein at least one of R^{A} and R^{B} is a substituted or an unsubstituted alkyl group comprising 1 to 30 carbon atoms.

13. The compound of claim 11, wherein R^{A} and the R^{B} form the spiro ring.

14-18. (canceled)

19. A compound represented by the following formula (3), 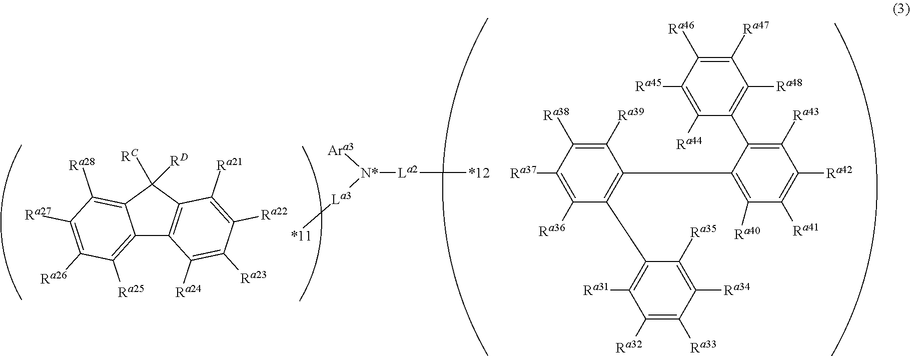
wherein N* is a central nitrogen atom,
L^{a2}, L^{a3}, *11, R^{a21} to R^{a24}, and R^{a25} to R^{a28} are each as defined in formula (1),
one selected from R^{a31} to R^{a48} is a single bond bonded to *12, and R^{a31} to R^{a48} which are not a single bond bonded to *12 are a hydrogen atom,
provided that two adjacent ones selected from R^{a31} to R^{a48} which are not a single bond bonded to *12 are bonded to each other to form a benzene ring, or R^{a31} to R^{a48} are not bonded to each other and therefore do not form a ring,
R^{C} and R^{D} are bonded to each other to form a substituted or an unsubstituted spiro ring with the carbon atom at the 9-position of the fluorene skeleton, and
Ar^{a3} is a phenyl group or a naphthyl group optionally substituted with a triarylsilyl group comprising a substituted or an unsubstituted aryl group comprising 6 to 30 ring carbon atoms.

20-23. (canceled)

24. The compound of claim 1, comprising:
a deuterium atom.

25-26. (canceled)

27. An organic electroluminescent device comprising:
a cathode;
an anode; and
an organic layer between the cathode and the anode,
wherein the organic layer comprises a light emitting layer, and
wherein a layer of the organic layers comprises the compound of claim 1.

28. The organic electroluminescent device of claim 27, wherein the organic layer comprises a hole transporting band between the anode and the light emitting layer, and
wherein the hole transporting band comprises the compound.

29. The organic electroluminescent device of claim 28, wherein the hole transporting band comprises a first hole transporting layer on the anode side and a second hole transporting layer on the cathode side, and
wherein one or both of the first hole transporting layer and the second hole transporting layer comprise the compound.

30. The organic electroluminescent device of claim 29, wherein the light emitting layer and the second hole transporting layer are directly adjacent.

31. (canceled)

32. The organic electroluminescent device of claim 28, wherein the hole transporting band comprises a first hole transporting layer, a second hole transporting layer, and a third hole transporting layer, in this order from the anode side, and
wherein one layer of the first to the third hole transporting layers, two layers of the first to the third hole transporting layers, or all of the first to the third hole transporting layers comprise the compound.

33. The organic electroluminescent device of claim 32, wherein the light emitting layer and the third hole transporting layer are directly adjacent.

34. (canceled)

35. The organic electroluminescent device of claim 27, wherein the light emitting layer is a single layer.

36. (canceled)

37. The organic electroluminescent device of claim 27, wherein the light emitting layer comprises a fluorescent dopant material.

38. An electronic device, comprising:
the organic electroluminescent device of claim 27.
